(19) United States
(12) Patent Application Publication
(10) Pub. No.: US 2026/0044076 A1
(43) Pub. Date: Feb. 12, 2026

(54) SULFONIUM SALT TYPE MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/285,230

(22) Filed: Jul. 30, 2025

(30) Foreign Application Priority Data
Aug. 8, 2024 (JP) 2024-131989

(51) Int. Cl. G03F 7/004; C07C 381/12; C08F 22/10; G03F 7/00; G03F 7/039

(52) U.S. Cl. CPC G03F 7/0045; C07C 381/12; C08F 22/10; G03F 7/0046; G03F 7/0397; G03F 7/70033

(57) ABSTRACT

The sulfonium salt type monomer has the following formula (A). The sulfonium salt type monomer can be used for a chemically amplified resist composition having excellent solvent solubility, high sensitivity, high contrast, and excellent lithographic performances such as EL, LWR, CDU, and DOF particularly in photolithography using high-energy radiation such as KrF excimer laser, ArF excimer laser, electron beam (EB), and EUV.

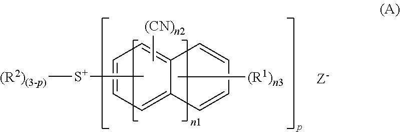

### CROSS-REFERENCE TO RELATED APPLICATION

This non-provisional application claims priority under 35 U.S.C. § 119 (a) on Patent Application No. 2024-131989 filed in Japan on Aug. 8, 2024, the entire contents of which are hereby incorporated by reference.

### TECHNICAL FIELD

The present invention relates to a sulfonium salt type monomer, a polymer, a chemically amplified resist composition, and a pattern forming process.

### BACKGROUND ART

To meet the demand for higher integration density and operating speed of LSIs, the effort to reduce the pattern rule is in rapid progress. In particular, the wide-spreading flash memory market and the demand for increased storage capacities drive forward the miniaturization technology. As the advanced miniaturization technology, manufacturing of microelectronic devices at the 65-nm node by the ArF lithography has been implemented in a mass scale, and manufacturing of 45-nm node devices by the next generation ArF immersion lithography is approaching to the verge of high-volume application. The candidates for the future generation 32-nm node include ultra-high NA lens immersion lithography using a liquid having a higher refractive index than water in combination with a high refractive index lens and a high refractive index resist film, extreme ultraviolet (EUV) lithography of 13.5 nm wavelength, double exposure (double patterning lithography) of ArF lithography, and the like, and studies are being conducted.

As miniaturization progresses, approaching to the diffraction limit of light, light contrast lowers. In the case of a positive resist film, a lowering of light contrast leads to reductions of resolution and focus margin of hole and trench patterns.

Roughness of a line width (LWR) of a line pattern and dimensional uniformity (CDU) of a hole pattern have been regarded as problems along with miniaturization of a pattern. It is pointed out that these factors are affected by the segregation or aggregation of a base polymer and an acid generator and the acid diffusion. There is a tendency that as the resist film becomes thinner, LWR becomes greater, and a film thickness reduction to comply with the progress of miniaturization causes a degradation of LWR, which becomes a serious problem.

In the EUV lithography resist composition, it is necessary to simultaneously achieve high sensitivity, high resolution, and low LWR. If the acid diffusion distance is reduced, LWR is reduced, but sensitivity becomes lower. For example, as the post exposure bake (PEB) temperature is lowered, the outcome is a reduced LWR, but a lower sensitivity. If the amount of the quencher added is increased, the outcome is a reduced LWR, but a lower sensitivity. It is necessary to overcome the tradeoff relation between sensitivity and LWR.

In order to suppress acid diffusion, a resist compound containing a repeat unit derived from an onium salt of sulfonic acid having a polymerizable unsaturated bond has been proposed (Patent Document 1). Such a so-called polymer-bound acid generator is characterized by very short acid diffusion because a polymer-type sulfonic acid is generated by exposure. The sensitivity can also be improved by increasing the ratio of the acid generator. If the amount of the additive type acid generator added is increased, the sensitivity is increased, but in this case, the acid diffusion distance is also increased. Since the acid diffuses non-uniformly, LWR and CDU deteriorate as the acid diffusion increases. It can be said that the polymer-type acid generator has high ability in the balance of sensitivity, LWR, and CDU.

Iodine atoms absorb a very large amount of EUV having a wavelength 13.5 nm, and have been confirmed to be capable of generating secondary electrons during exposure, and attracted attention in the EUV lithography. Patent Document 2 describes a photoacid generator in which an iodine atom is introduced into an anion, and Patent Document 3 describes a polymerizable group-containing photoacid generator in which an iodine atom is introduced into an anion. As a result, although improvement in lithographic performances to some extent has been confirmed, iodine atoms do not have high organic solvent solubility, and there is a concern about deposition in the solvent.

Patent Documents 4 and 5 describe photoacid generators in which a pentafluorosulfanyl group (—SF₅ group) or a trifluoromethoxy group (—OCF₃ group) is introduced into a cation. As a result, although lithographic performance has been improved to some extent, there is still room for improvement, and development of a resist material effective for further fine pattern formation is desired.

### CITATION LIST

Patent Document 1: JP 4425776
Patent Document 2: JP 6720926
Patent Document 3: JP 6973274
Patent Document 4: WO 2023/223624
Patent Document 5: JP-A 2022-59112

### SUMMARY OF THE INVENTION

In the field of acid-catalyzed chemically amplified resist composition, it is desired to develop a resist composition which has higher sensitivity and can improve lithographic performance such as exposure tolerance (EL), LWR, CDU, and depth of focus (DOF).

It is an object of the present invention to provide a sulfonium salt type monomer which is used for a chemically amplified resist composition having excellent solvent solubility, high sensitivity, high contrast, and excellent lithographic performances such as EL, LWR, CDU, and DOF particularly in photolithography using high-energy radiation such as KrF excimer laser, ArF excimer laser, electron beam (EB), and EUV, a polymer including repeat units derived from the sulfonium salt type monomer, a chemically amplified resist composition comprising the polymer, and a pattern forming process using the chemically amplified resist composition.

The present inventors have conducted intensive studies to acid the above object, and as a result, have found that by using, as a polymer-bound acid generator, a polymer containing repeat units derived from a sulfonium salt type monomer composed of a sulfonium cation having a cyano group and an aromatic sulfonic acid anion having an aromatic vinyl structure and an iodine atom, a chemically amplified resist composition having high sensitivity, improved lithographic performance such as EL, LWR, CDU, or DOF, high contrast, and high resolution is obtained, thereby completing the present invention.

That is, the present invention provides the following sulfonium salt type monomer, polymer, chemically amplified resist composition, and pattern forming process.

1. A sulfonium salt type monomer having the following formula (A):

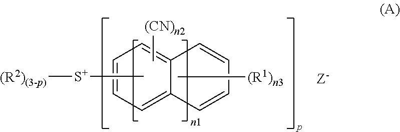

wherein p is 1, 2, or 3, n1 is 0 or 1, n2 is 1 or 2, n3 is 0, 1, 2, or 3, provided that, when n1 is 0, 1≤n2+n3≤5, and when n1 is 1, 1≤n2+n3≤7,
R¹ is a halogen atom, a nitro group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, when n3 is 2 or 3, respective R¹ may be the same as or different from each other, and two R′'s may bond together to form a ring with the carbon atom to which they are attached,
R² is a halogen atom, or a C₁-C₃₀ hydrocarbyl group which may contain a heteroatom, when p is 1, two R²'s may be the same as or different from each other, two of three substituents bonded to S⁺ may bond together to form a ring with the sulfur atom to which they are bonded, and
Z⁻ is an aromatic sulfonic acid anion having an aromatic vinyl structure.

2. The sulfonium salt type monomer according to 1, which has the following formula (A1):

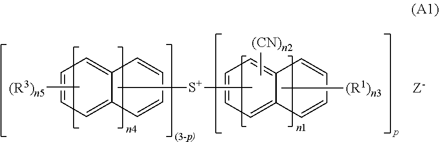

wherein p, n1 to n3, R¹, and Z⁻ are as defined above,
n4 is 0 or 1, n5 is 0, 1, 2, 3, 4, or 5,
R³ is a halogen atom, a nitro group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n5 is 2, 3, 4, or 5, respective R³ may be the same as or different from each other, and two R³'s may bond together to form a ring with the carbon atom to which they are attached.

3. The sulfonium salt type monomer according to 1 or 2, wherein Z⁻ is an anion having the following formula (Z):

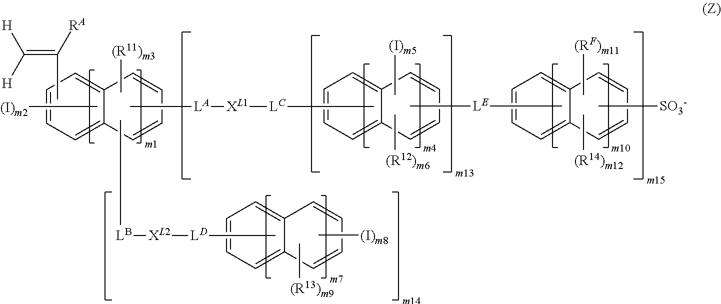

wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1, 2, 3, or 4, m9 is 0, 1, 2, or 3. m10 is 0 or 1, m11 is 0, 1, 2, 3, or 4, m12 is 0, 1, 2, or 3, m13 is 0 or 1, m14 is 0 or 1, m15 is 0 or 1, provided that, when m1 is 0, 0≤m2+m3+m14≤4, and when m1 is 1, 0≤m2+m3+m14≤6, when m4 is 0, 0≤m5+m6≤4, and when m4 is 1, 0≤m5+m6≤6, when m7 is 0, 0≤m8+m9≤5, and when m7 is 1, 0≤m8+m9≤7, and when m10 is 0, 0≤m11+m12≤4, and when m10 is 1, 0≤m11+m12≤6, and 1≤m2+m5+m8≤4,
R^{A} is a hydrogen atom, a fluorine atom, a methyl group, or a trifluoromethyl group,
R¹¹, R¹², and R₁₃ are each independently a halogen atom other than an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, when m3 is 2 or 3, respective R¹¹ may be the same as or different from each other, and two R¹¹'s may bond together to form a ring with the carbon atom to which they are attached, when m6 is 2 or 3, respective R¹² may be the same as or different from each other, and two R¹²'s may bond together to form a ring with the carbon atom to which they are attached, when m9 is 2 or 3, respective R¹³ may be the same as or different from each other, and two R¹³ may bond together to form a ring with the carbon atom to which they are attached,
R¹⁴ is a halogen atom other than a fluorine atom and an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m12 is 2 or 3, respective R¹⁴ may be the same as or different from each other, and two R¹⁴'s may bond together to form a ring with the carbon atom to which they are attached,
R^{F} is a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when m11 is 2, 3, or 4, respective R^{F} may be the same as or different from each other,
L^{A}, L^{B}, L^{C}, L^{D}, and L^{E} are each independently a single bond, an ether bond, an ester bond, a sulfonic acid ester bond, an amide bond, a sulfonic acid amide bond, a carbonate bond, or a carbamate bond,
X^{L1} and X^{L2} are each independently a single bond or a C₁-C₄₀ hydrocarbylene group which may contain a heteroatom,
provided that m13 and m14 are not 0 at the same time, and L^{A}, L^{B}, L^{C}, L^{D}, X^{L1}, and X^{L2} are not a single bond at the same time.

4. The sulfonium salt type monomer according to 3, wherein m15 is 1.

5. A monomeric photoacid generator comprising the sulfonium salt type monomer according to any one of 1 to 4.

6. A polymer comprising repeat units derived from the monomeric photoacid generator according to 5.

7. The polymer according to 6, further comprising at least one selected from repeat units having the following formula (a1), repeat units having the following formula (a2), and repeat units having the following formula (a3):

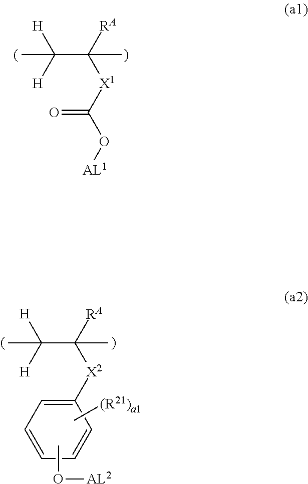

wherein R^{A} is each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom in the backbone,
R²¹ is a halogen atom, a cyano group, a hydroxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, respective R²¹ may be the same as or different from each other,
AL¹ and AL² are each independently an acid labile group, and
a1 is 0, 1, 2, 3, or 4, and

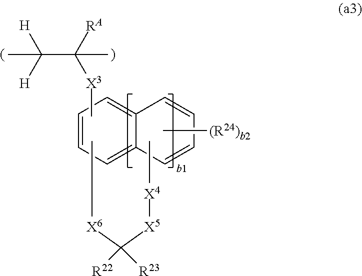

wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(—O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or groups obtained by combining these,
X⁵ and X⁶ are each independently an oxygen atom or a sulfur atom, provided that X⁴ and X⁶ bond to adjacent carbon atoms of an aromatic ring,
R²² and R²³ are each independently a hydrogen atom, or a C₁-C₂₀ hydrocarbyl group which may contain a heteroatom, R²² and R²³ may bond together to form a ring with the carbon atom to which they are attached,
R²⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group, and when b2 is 2 or more, respective R²⁴ may be the same as or different from each other, and a plurality of R²⁴'s may bond together to form a ring with the carbon atom of an aromatic ring to which they are attached.

8. The polymer according to 6 or 7, further comprising at least one selected from repeat units having the following formula (b1) and repeat units having the following formula (b2):

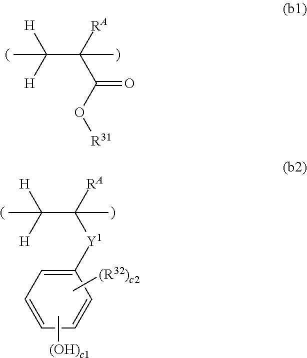

wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—),
R³² is a halogen atom, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2, 3, or 4, respective R³² may be the same as or different from each other,
c1 is 1, 2, 3, or 4, c2 is 0, 1, 2, 3, or 4, provided that 1≤cl+c2≤5.

9. A chemically amplified resist composition comprising (A) a base polymer containing the polymer according to any one of 6 to 8.

10. The chemically amplified resist composition according to 9, further comprising (B) an organic solvent.

11. The chemically amplified resist composition according to claim 9 or 10, further comprising (C) a quencher.

12. The chemically amplified resist composition according to any one of 9 to 11, further comprising (D) a photoacid generator.

13. The chemically amplified resist composition according to any one of 9 to 12, further comprising (E) a surfactant.

14. A pattern forming process comprising the steps of: applying the chemically amplified resist composition according to any one of 9 to 13 onto a substrate to form a resist film thereon; exposing the resist film to high-energy radiation; and developing the exposed resist film in a developer.

15. The pattern forming process according to 14, wherein the high-energy radiation is KrF excimer laser, ArF excimer laser, EB, or EUV having a wavelength of 3 to 15 nm.

### Advantageous Effects of the Invention

When a pattern is formed using a chemically amplified resist composition comprising a polymer, which functions as a photoacid generator, including repeat units derived from the sulfonium salt type monomer of the present invention, a resist pattern having high contrast, favorable sensitivity, and excellent lithographic performances such as EL, LWR, CDU, and DOF can be formed.

### DESCRIPTION OF THE PREFERRED EMBODIMENT

Hereinafter, the present invention is described in detail. In the following description, depending on the structure having the chemical formula, an asymmetric carbon may exist, and an enantiomer or a diastereomer may exist, but in that case, those isomers have one formula as a representative. These isomers may be used alone, or may be used as a mixture of two or more thereof.

### Sulfonium Salt Type Monomer

A sulfonium salt type monomer of the present invention has the following formula (A):

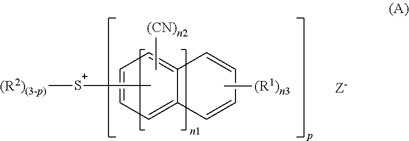

wherein p is 1, 2, or 3.

In formula (A), n1 is 0 or 1. When n1 is 0, the relevant structure is a benzene ring, and when n1 is 1, the relevant structure is a naphthalene ring, but from the viewpoint of solvent solubility, the relevant structure is preferably a benzene ring in which n1 is 0. n2 is 1 or 2. From the viewpoint of raw material availability, n2 is preferably 1. n3 is 0, 1, 2, or 3. From the viewpoint of raw material availability, n3 is preferably 0, 1, or 2. Provided that, when n1 is 0, 1≤n2+n3≤5, and when n1 is 1, 1≤n2+n3≤7,

In formula (A), R¹ is a halogen atom, a nitro group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Examples of the halogen atom include a fluorine atom, a chlorine atom, a bromine atom, and an iodine atom. The hydrocarbyl moiety of the hydrocarbyl group, the hydrocarbyloxy group, and the hydrocarbylthio group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ allyl groups such as a phenyl group and a naphthyl group; C₇-C₂₀ aralkyl groups such as a benzyl group, a 1-phenylethyl group, and a 2-phenylethyl group; and groups obtained by combining these. Of these, an aryl group is preferable. Some or all of the hydrogen atoms in the hydrocarbyl group may be substituted with a group containing a heteroatom such as an oxygen atom, a sulfur atom, a nitrogen atom, or a halogen atom, some of—CH2-in the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. When n3 is 2 or 3, respective R¹ may be the same as or different from each other. When n3 is 2 or 3, a plurality of R¹'s may bond together to form a ring with the carbon atom to which they are attached. The ring is preferably a 5- to 8-membered ring.

In formula (A), R² is a halogen atom, or a C₁-C₃₀ hydrocarbyl group which may contain a heteroatom. When p is 1, two R²'s may be the same as or different from each other.

Specific examples of the halogen atom represented by R² include a fluorine atom, a chlorine atom, a bromine atom, and an iodine atom.

The hydrocarbyl group represented by R² may be saturated or unsaturated, and may be straight, branched, or cyclic. Specific examples thereof include C₁-C₃₀ alkyl groups such as a methyl group, an ethyl group, a n-propyl group, an isopropyl group, a n-butyl group, an isobutyl group, a sec-butyl group, and a tert-butyl group; C₃-C₃₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₃₀ alkenyl groups such as a vinyl group, a 1-propenyl group, a 2-propenyl group, a butenyl group, and a hexenyl group; C₃-C₃₀ cyclic unsaturated hydrocarbyl groups such as a cyclohexenyl group; C₆-C₃₀ aryl groups such as a phenyl group, a naphthyl group, and a thienyl group; C₇-C₃₀ aralkyl groups such as a benzyl group, a 1-phenylethyl group, and a 2-phenylethyl group; and groups obtained by combining these, and an aryl group is preferable. Some or all of hydrogen atoms of the hydrocarbyl group may be substituted with a group containing a heteroatom such as an oxygen atom, a sulfur atom, a nitrogen atom, or a halogen atom, and some of —CH₂— of the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), or a haloalkyl group.

Two of three substituents bonded to S⁺ may bond together to form a ring with the sulfur atom to which they are bonded. In this case, specific examples of the structure of the ring are those having the following formula:

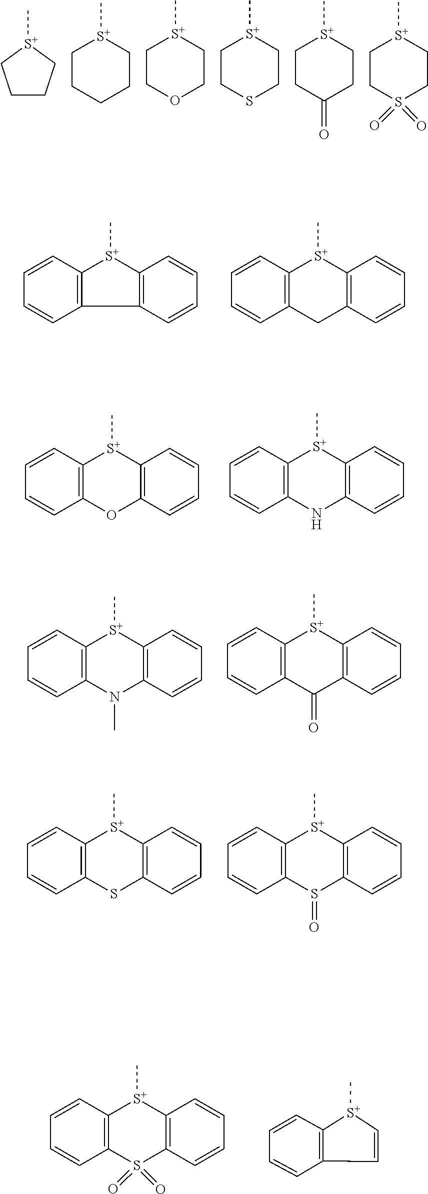

wherein the broken line denotes a point of attachment.

The sulfonium salt type monomer having formula (A) preferably has the following formula (A1):

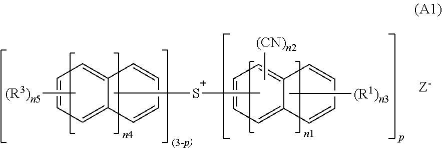

wherein p, n1 to n3, and R¹ are the same as described above, and Z⁻ is described below.

In formula (A1), n4 is 0 or 1. When n4 is 0, the relevant structure is a benzene ring, and when n4 is 1, the relevant structure is a naphthalene ring, but from the viewpoint of solvent solubility, the relevant structure is preferably a benzene ring in which n4 is 0. n5 is 0, 1, 2, 3, 4, or 5. From the viewpoint of raw material availability, n5 is preferably 0, 1, or 2.

In formula (A1), R³ is a halogen atom, a nitro group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. The hydrocarbyl moiety of the hydrocarbyl group, the hydrocarbyloxy group, and the hydrocarbylthio group may be saturated or unsaturated and straight, branched, or cyclic. Specific examples thereof are as exemplified above for the hydrocarbyl group represented by R¹, but not limited thereto. When n5 is 2, 3, 4, or 5, respective R³ may be the same as or different from each other, and two R³'s may bond together to form a ring with the carbon atom to which they are attached.

Specific examples of the cation of the sulfonium salt type monomer having formula (A) are shown below, but not limited thereto.

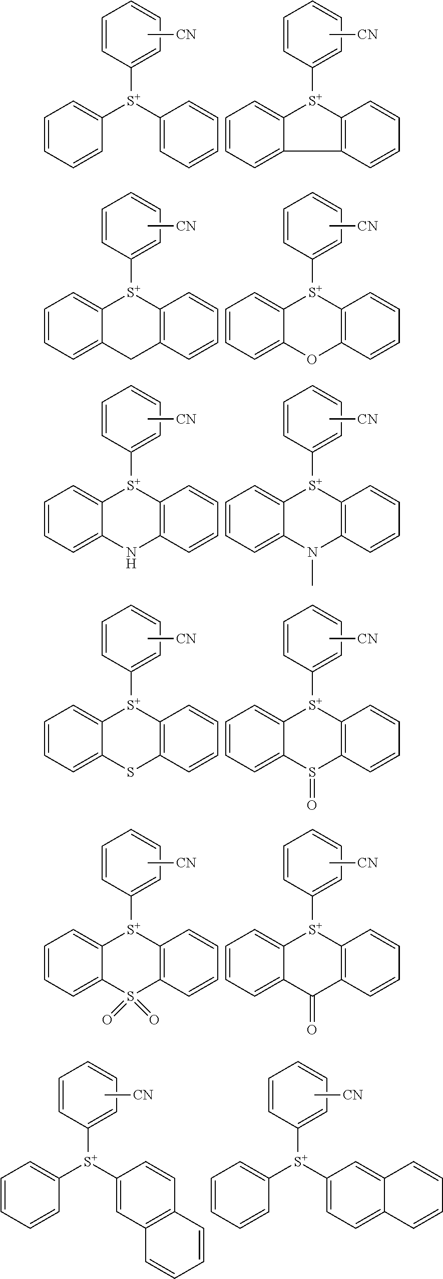

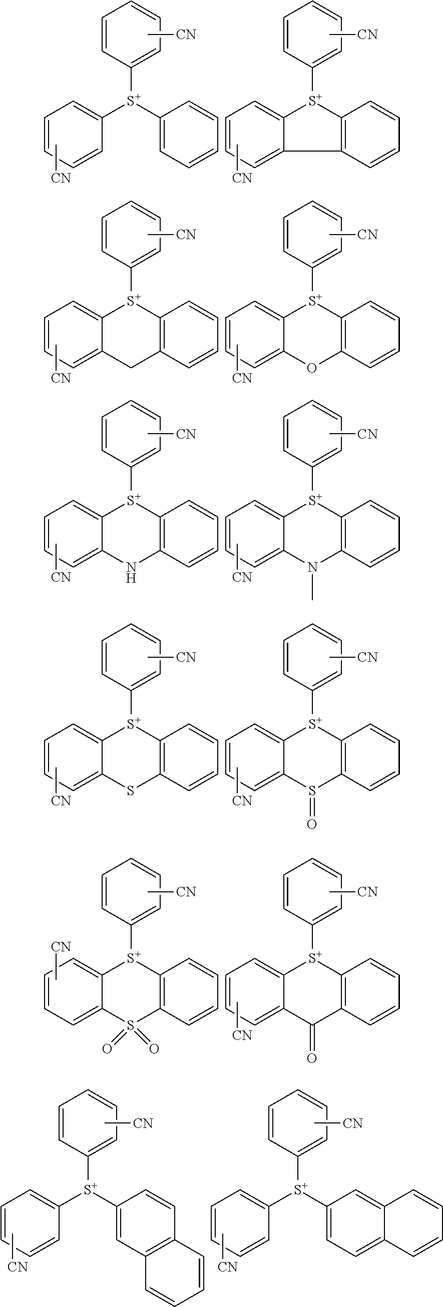

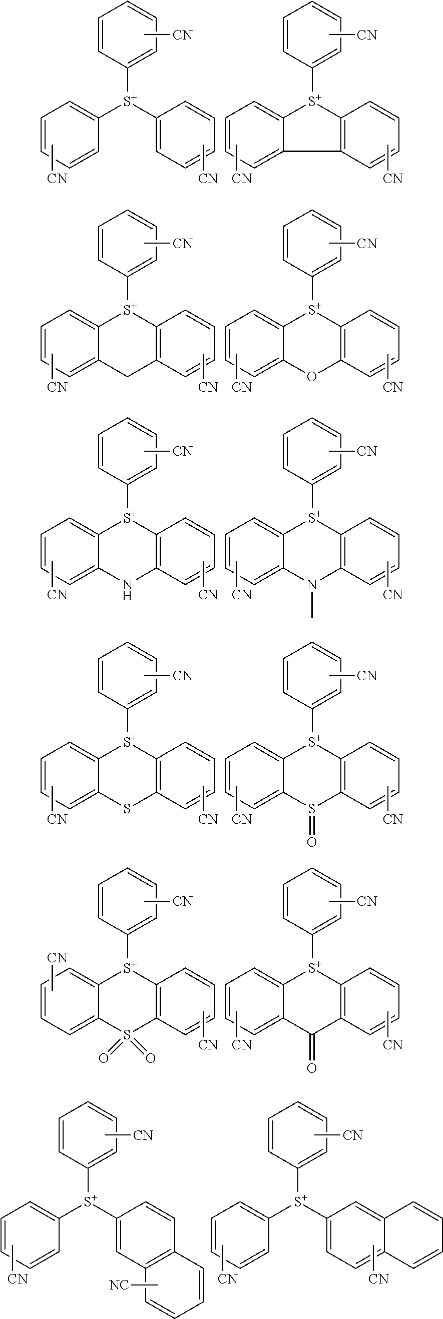

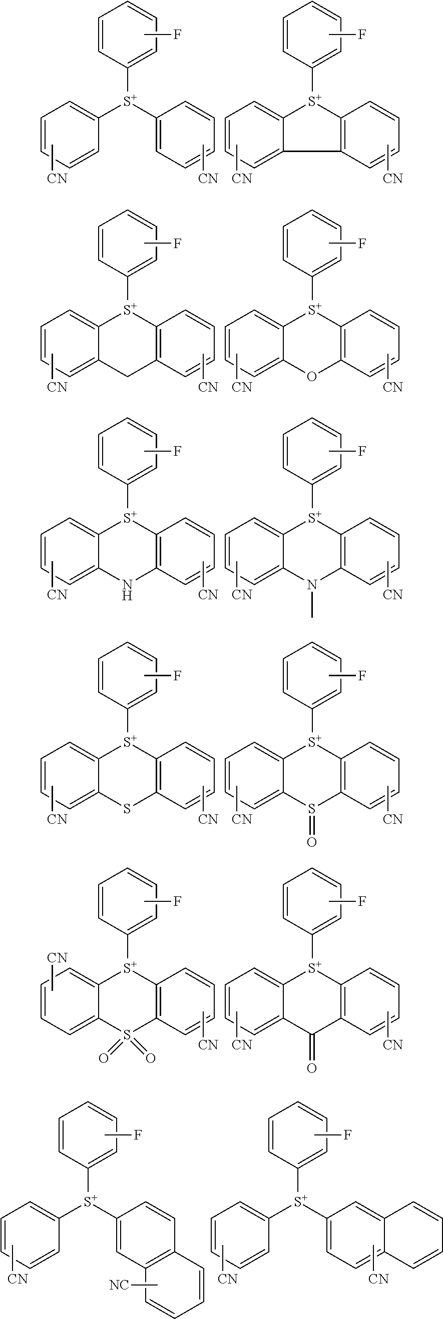

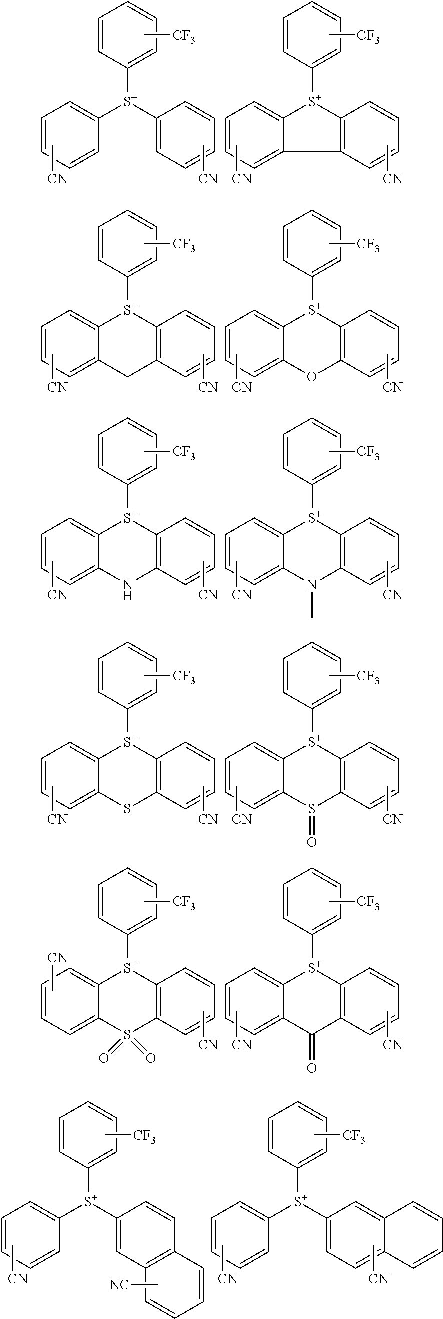

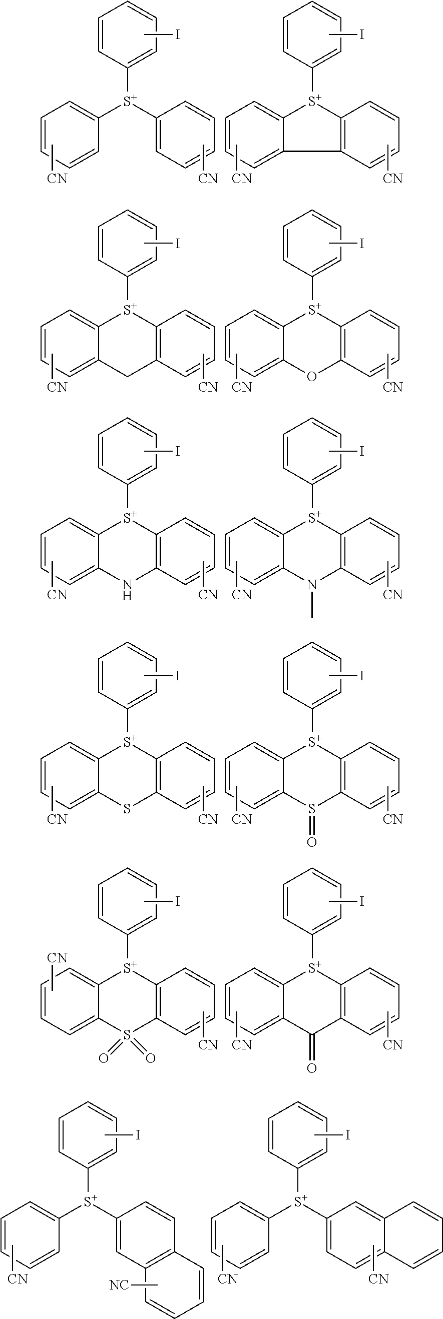

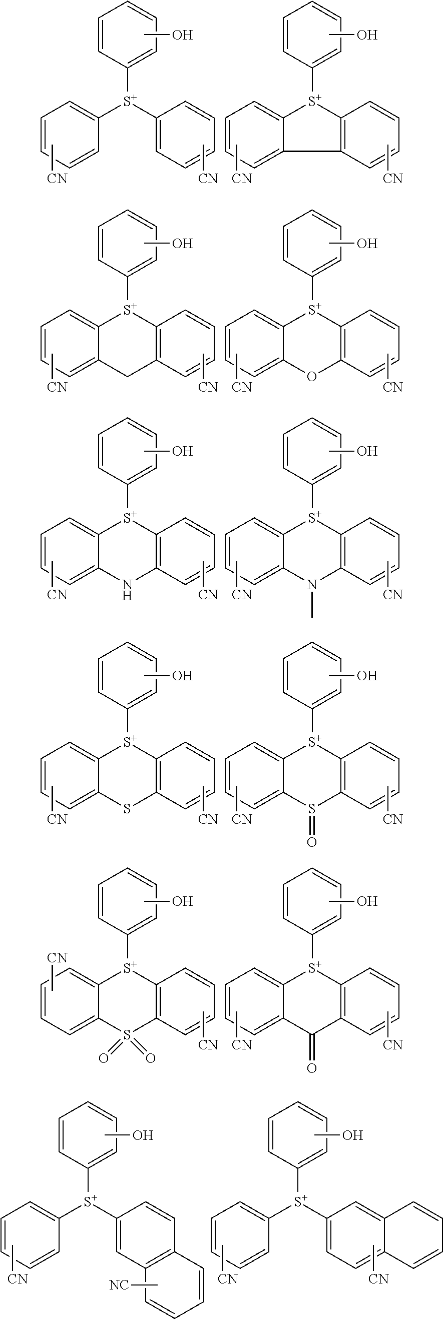

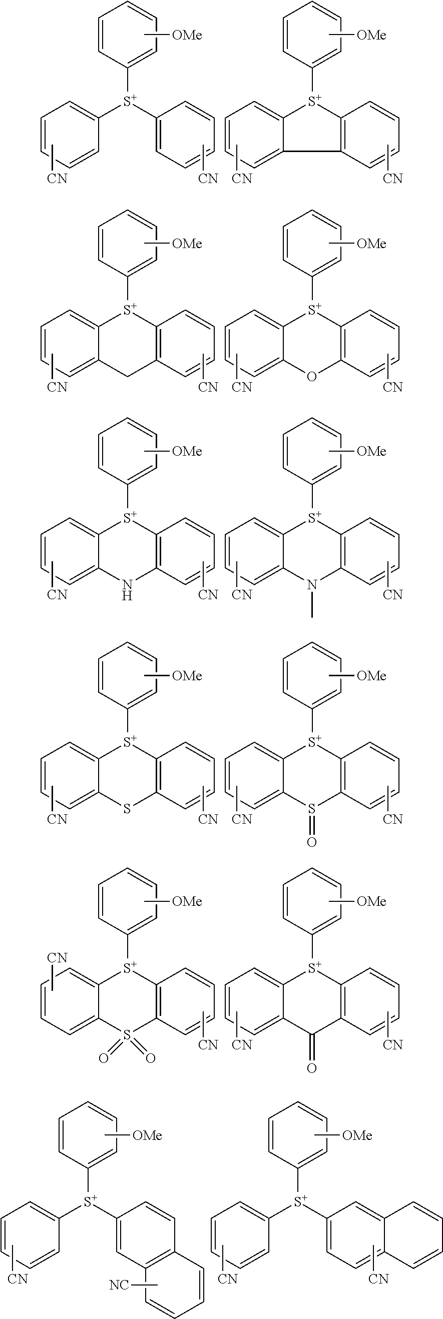

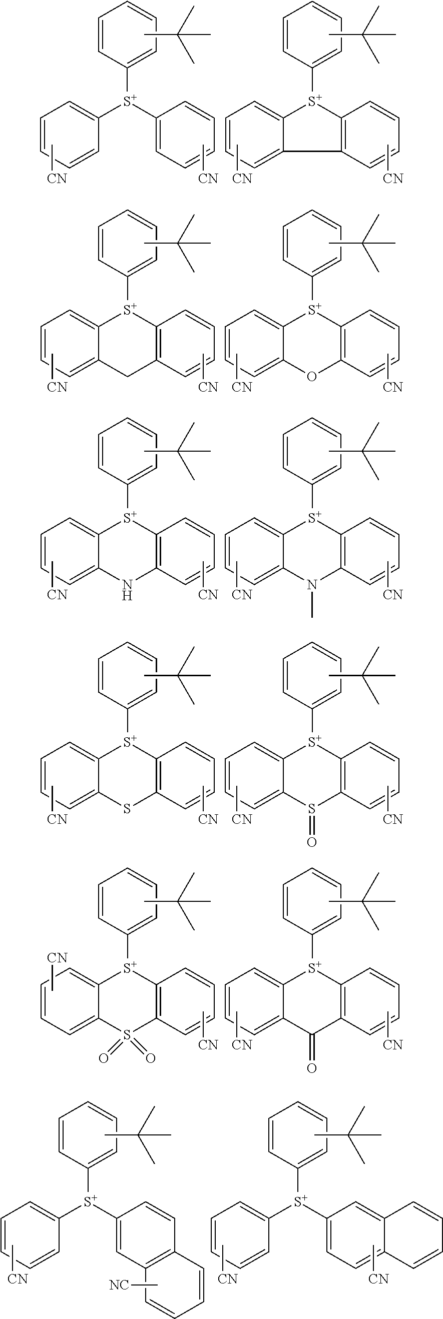

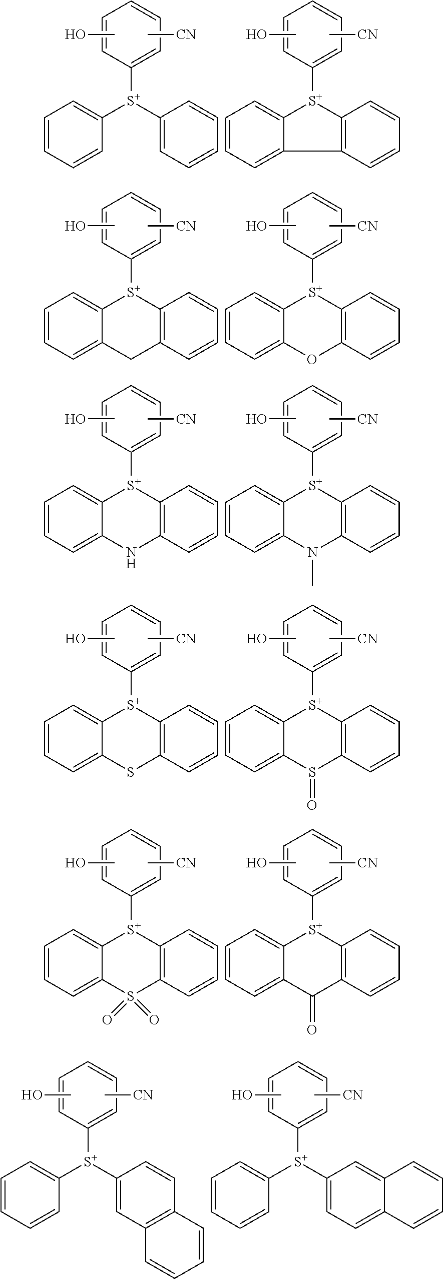

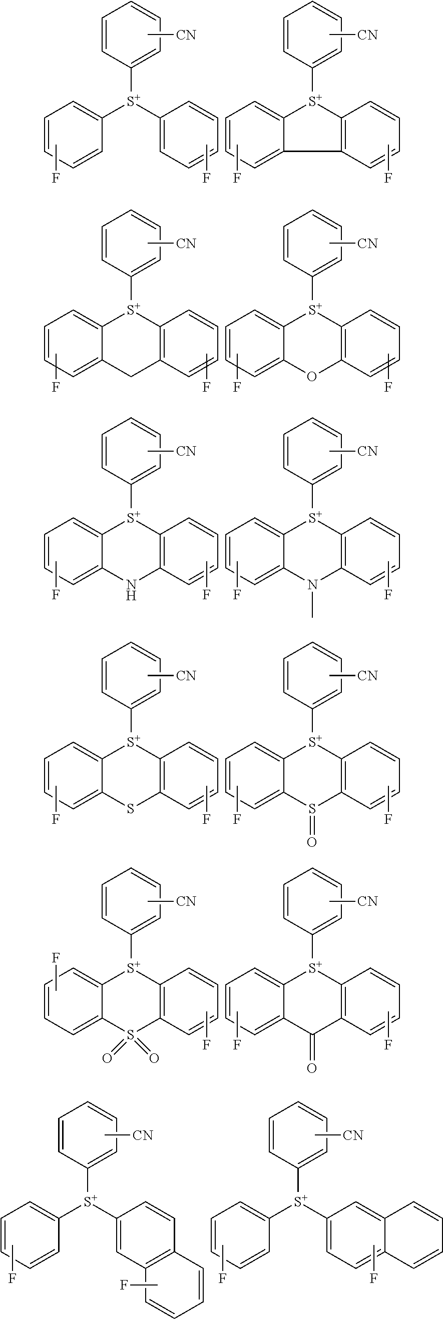

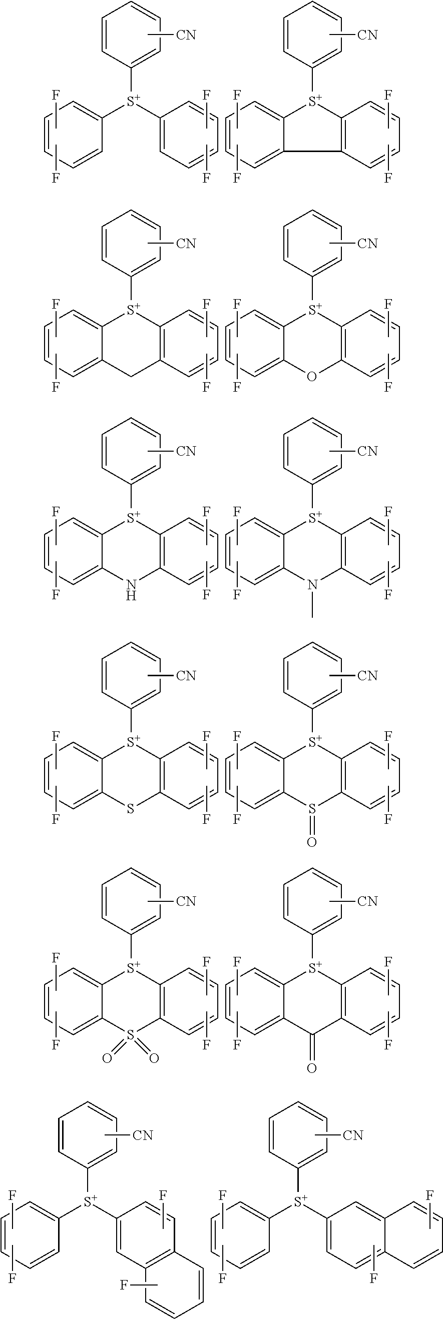

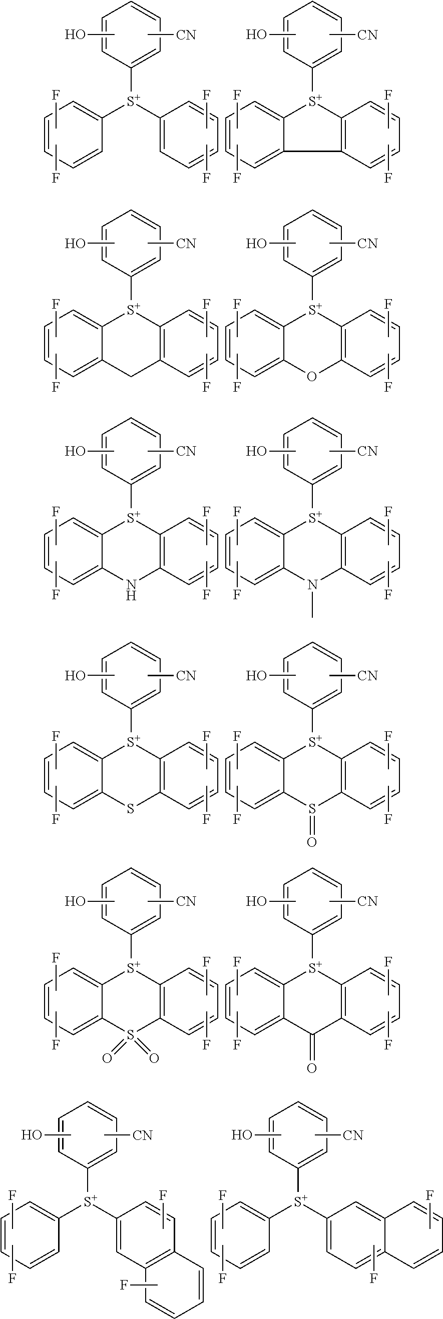

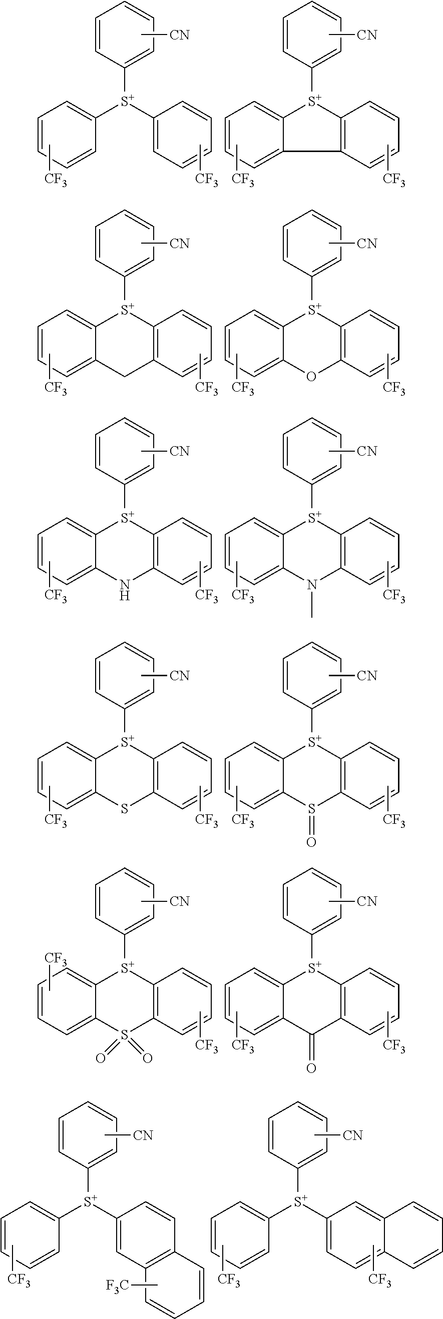

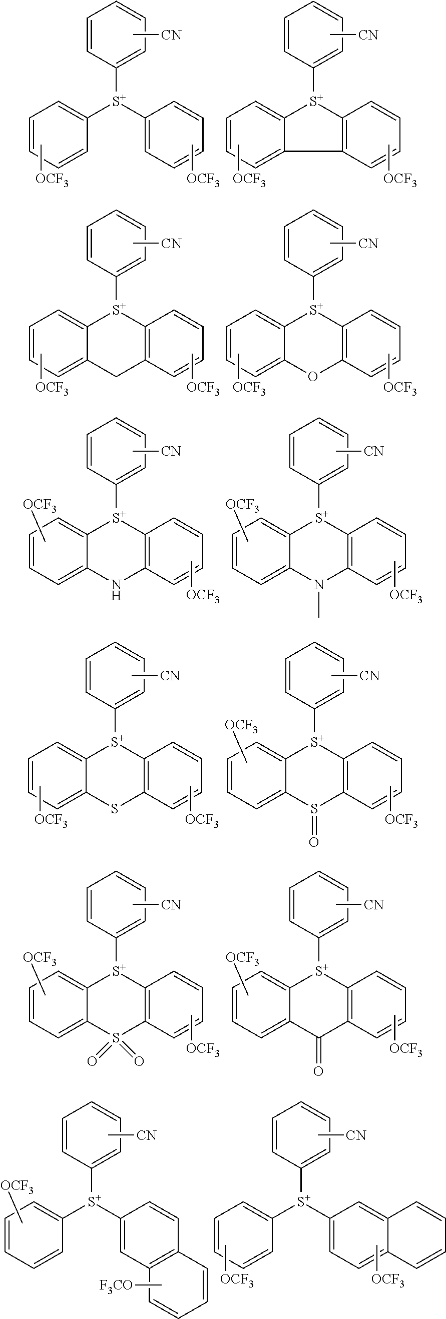

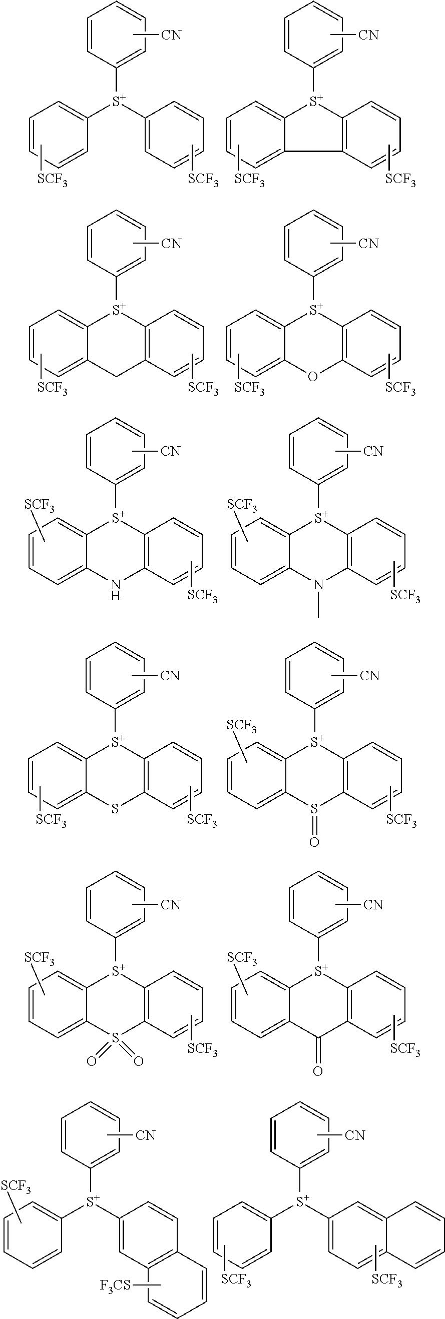

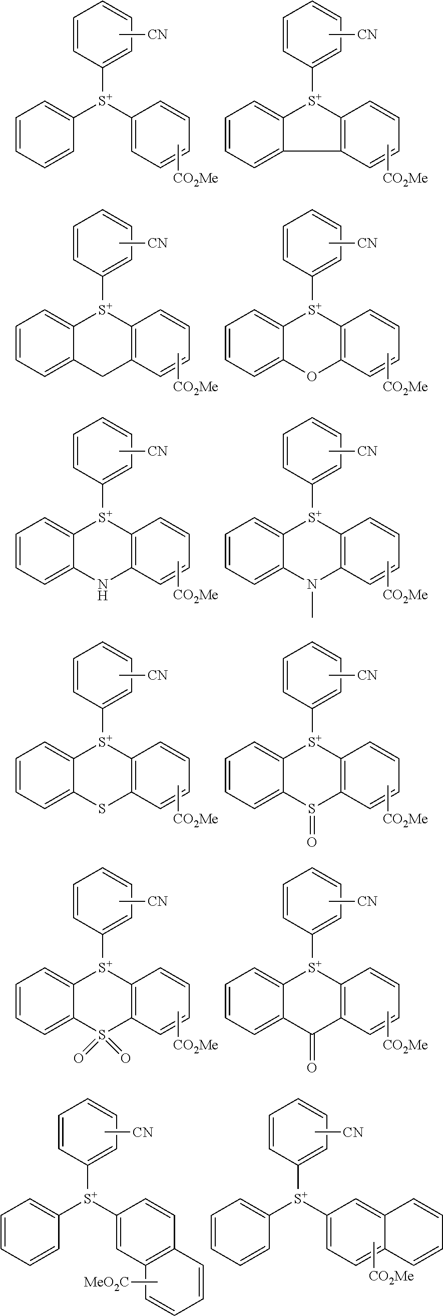

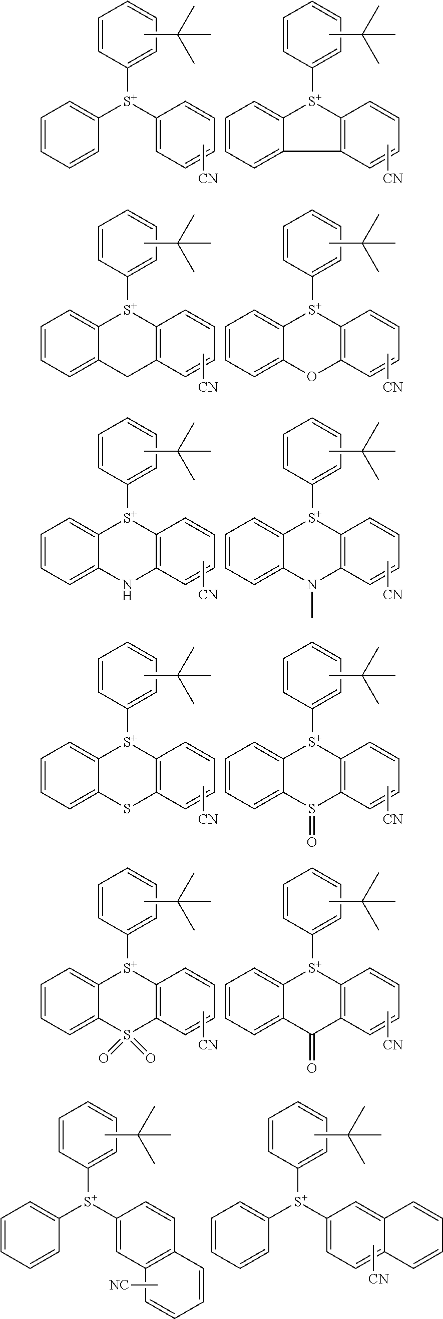

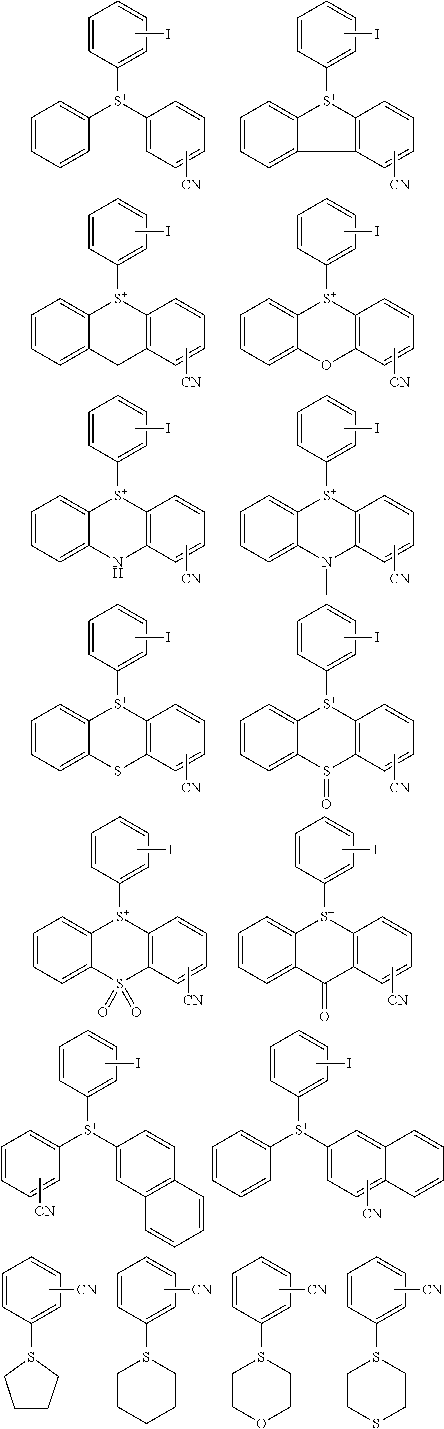

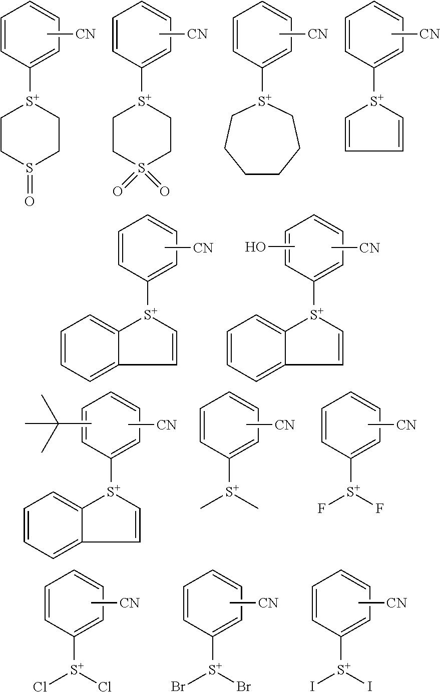

In formula (A), Z⁻ is an aromatic sulfonic acid anion having an aromatic vinyl structure. The aromatic sulfonic acid anion preferably has the following formula (Z):

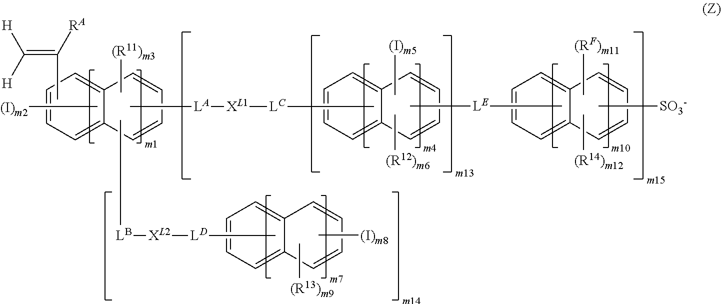

In formula (Z), m1 is 0 or 1. When m1 is 0, the relevant structure is a benzene ring, and when m1 is 1, the relevant structure is a naphthalene ring, but from the viewpoint of solvent solubility, the relevant structure is preferably a benzene ring in which m1 is 0. m2 is 0, 1, 2, 3, or 4. From the viewpoint of raw material availability, m2 is preferably 0, 1, 2, or 3, more preferably 0, 1, or 2, and further preferably 0 or 1. m3 is 0, 1, 2, or 3.

In formula (Z), m4 is 0 or 1. When m4 is 0, the relevant structure is a benzene ring, and when m4 is 1, the relevant structure is a naphthalene ring, but from the viewpoint of solvent solubility, the relevant structure is preferably a benzene ring in which m4 is 0. m5 is 0, 1, 2, 3, or 4. From the viewpoint of raw material availability, m5 is preferably 0, 1, 2, or 3 and more preferably 0, 1, or 2. m6 is 0, 1, 2, or 3.

In formula (Z), m7 is 0 or 1. When m7 is 0, the relevant structure is a benzene ring, and when m7 is 1, the relevant structure is a naphthalene ring, but from the viewpoint of solvent solubility, the relevant structure is preferably a benzene ring in which m7 is 0. m8 is 1, 2, 3, or 4. From the viewpoint of raw material availability, m8 is preferably 1, 2, or 3 and more preferably 1 or 2. m9 is 0, 1, 2, or 3.

In formula (Z), m10 is 0 or 1. When m10 is 0, the relevant structure is a benzene ring, and when m10 is 1, the relevant structure is a naphthalene ring, but from the viewpoint of solvent solubility, the relevant structure is preferably a benzene ring in which m10 is 0. m11 is 0, 1, 2, 3, or 4. m12 is 0, 1, 2, or 3.

In formula (Z), m13 is 0 or 1. m14 is 0 or 1,

In formula (Z), m15 is 0 or 1. From the viewpoint of structural modification, m15 is preferably 1.

Provided that, when m1 is 0, 0≤m2+m3+m14≤4, and when m1 is 1, 0≤m2+m3+m14≤6. When m4 is 0, 0≤m5+m6≤4, and when m4 is 1, 0≤m5+m6≤6. When m7 is 0, 0≤m8+m9≤5, and when m7 is 1, 0≤m8+m9≤7. When m10 is 0, 0≤m11+m12≤4, and when m10 is 1, 0≤ml1+m12≤6. With respect to the number of iodine atoms in the anion, the larger the number of iodine atoms is, the more the absorption for EUV is particularly enhanced, but since there is a concern that the solvent solubility becomes poor and precipitation occurs in the resist composition, 1≤m2+m5+m8≤4 is preferable.

In formula (Z), R^{A} is a hydrogen atom, a fluorine atom, a methyl group, or a trifluoromethyl group. R^{A} is preferably a hydrogen atom or a methyl group, and more preferably a hydrogen atom.

In formula (Z), R¹¹ is a halogen atom other than an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. The halogen atom other than an iodine atom is preferably a fluorine atom, a chlorine atom, or a bromine atom and more preferably a fluorine atom. The hydrocarbyl moiety of the hydrocarbyl group, the hydrocarbyloxy group, and the hydrocarbylthio group may be saturated or unsaturated and straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ allyl groups such as a phenyl group and a naphthyl group; C₇-C₂₀ aralkyl groups such as a benzyl group, a 1-phenylethyl group, and a 2-phenylethyl group; and groups obtained by combining these. Some or all of the hydrogen atoms in the hydrocarbyl group may be substituted with a group containing a heteroatom such as an oxygen atom, a sulfur atom, a nitrogen atom, or a halogen atom, some of—CH₂-in the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. When m3 is 2 or 3, respective R¹¹ may be the same as or different from each other.

When m3 is 2 or 3, two R¹¹'s may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring may be substituted with a group containing a heteroatom such as an oxygen atom, a sulfur atom, a nitrogen atom, or a halogen atom, some of—CH₂-in the ring may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

In formula (Z), R¹² is a halogen atom other than an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Specific examples of the halogen atom other than an iodine atom include a fluorine atom, a chlorine atom, and a bromine atom. The hydrocarbyl moiety of the hydrocarbyl group, the hydrocarbyloxy group, and the hydrocarbylthio group may be saturated or unsaturated and straight, branched, or cyclic. Specific examples thereof are as exemplified above for the hydrocarbyl group represented by R¹¹, but not limited thereto. When m6 is 2 or 3, respective R¹² may be the same as or different from each other.

When m6 is 2 or 3, two R¹²'s may bond together to form a ring with the carbon atom to which they are attached. The ring is preferably a 5- to 8-membered ring.

In formula (Z), R¹³ is a halogen atom other than an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom. Specific examples of the halogen atom other than an iodine atom include a fluorine atom, a chlorine atom, and a bromine atom. The hydrocarbyl moiety of the hydrocarbyl group, the hydrocarbyloxy group, and the hydrocarbylthio group may be saturated or unsaturated and straight, branched, or cyclic. Specific examples thereof are as exemplified above for the hydrocarbyl group represented by R¹¹, but not limited thereto. When m9 is 2 or 3, respective R¹³ may be the same as or different from each other.

When m9 is 2 or 3, two R¹³'s may bond together to form a ring with the carbon atom to which they are attached. The ring is preferably a 5- to 8-membered ring.

In formula (Z), R¹⁴ is a halogen atom other than a fluorine atom and an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Specific examples of the halogen atom other than a fluorine atom and an iodine atom include a chlorine atom and a bromine atom. The hydrocarbyl moiety of the hydrocarbyl group, the hydrocarbyloxy group, and the hydrocarbylthio group may be saturated or unsaturated and straight, branched, or cyclic. Specific examples thereof are as exemplified above for the hydrocarbyl group represented by R¹¹, but not limited thereto. When m12 is 2 or 3, respective R¹⁴ may be the same as or different from each other.

When m12 is 2 or 3, two R¹⁴'s may bond together to form a ring with the carbon atom to which they are attached. The ring is preferably a 5- to 8-membered ring.

In formula (Z), R^{F} is a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among them, a fluorine atom, a trifluoromethyl group, a trifluoromethoxy group, or a trifluoromethylthio group is preferable, and a fluorine atom is more preferable. When m11 is 2, 3, or 4, respective R^{F} may be the same as or different from each other.

In formula (Z), L^{A}, L^{B}, L^{C}, L^{D}, and L^{E} are each independently a single bond, an ether bond, an ester bond, a sulfonic acid ester bond, an amide bond, a sulfonic acid amide bond, a carbonate bond, or a carbamate bond. Of these, L^{A} is preferably a single bond, an ether bond, an ester bond, or a sulfonic acid ester bond, and more preferably an ether bond, an ester bond, or a sulfonic acid ester bond. L^{B} is preferably a single bond, an ether bond, an ester bond, an amide bond, a sulfonic acid amide bond, or a sulfonic acid ester bond, and more preferably an ester bond or a sulfonic acid ester bond. L^{C} is preferably a single bond, an ether bond, an ester bond, an amide bond, or a sulfonic acid ester bond, and more preferably a single bond, an ether bond, or an ester bond. L^{D} is preferably a single bond, an ether bond, an ester bond, an amide bond, or a sulfonic acid ester bond, and more preferably a single bond, an ether bond, or an ester bond. L^{E} is preferably a single bond, an ether bond, an ester bond, or a sulfonic acid ester bond, and more preferably a single bond, an ether bond, or an ester bond.

When m14 is 1, L^{A} and L^{B} preferably bond to adjacent carbon atoms of the aromatic ring. At this time, since the substituent including a fluorosulfonic acid anion structure and the substituent including an aromatic ring substituted with an iodine atom are present at more spatially close positions, higher sensitivity is expected.

In formula (Z), X^{L1} and X¹² are each independently a single bond or a C₁-C₄₀ hydrocarbylene group which may contain a heteroatom. The hydrocarbylene group may be straight, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group, which may contain a heteroatom, represented by X^{L1} and X^{L2} are shown below, but not limited thereto. In the following formula. * designates a point of attachment to L^{A} and L^{C} or L^{B} and L^{D}.

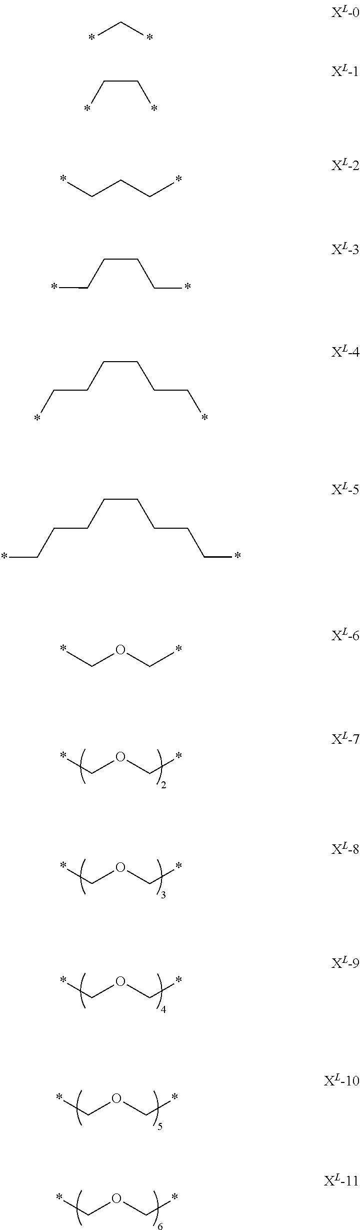

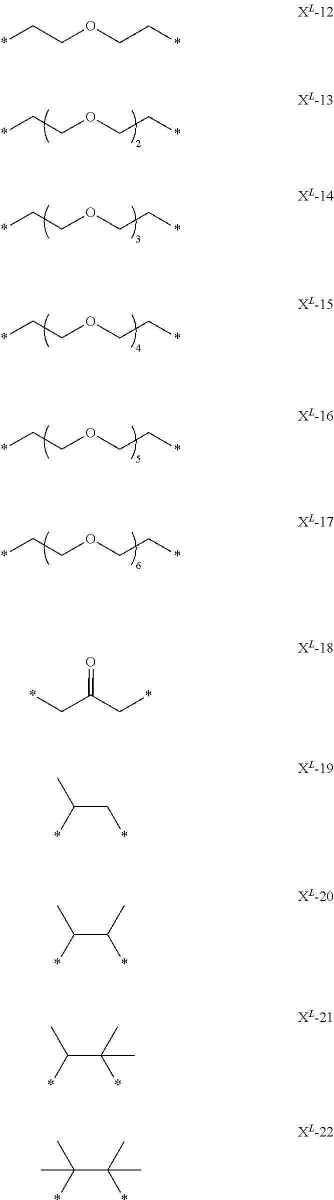

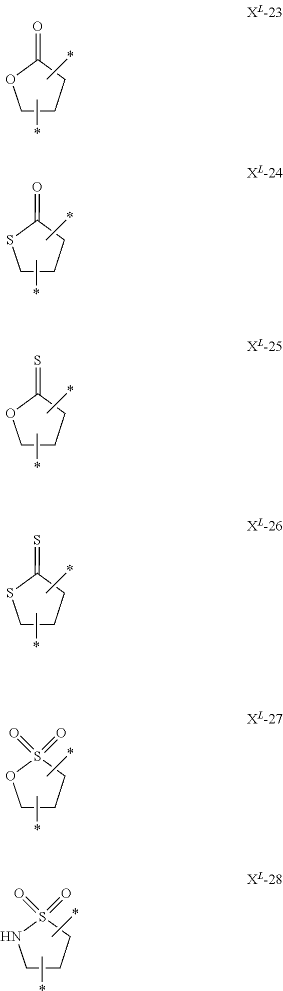

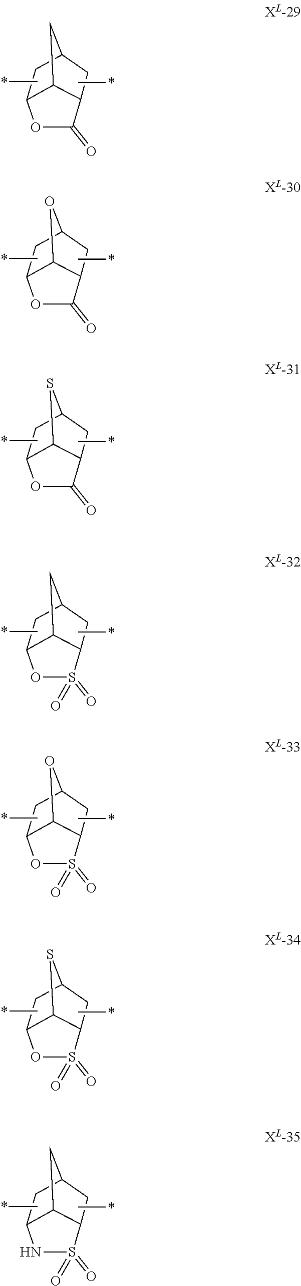

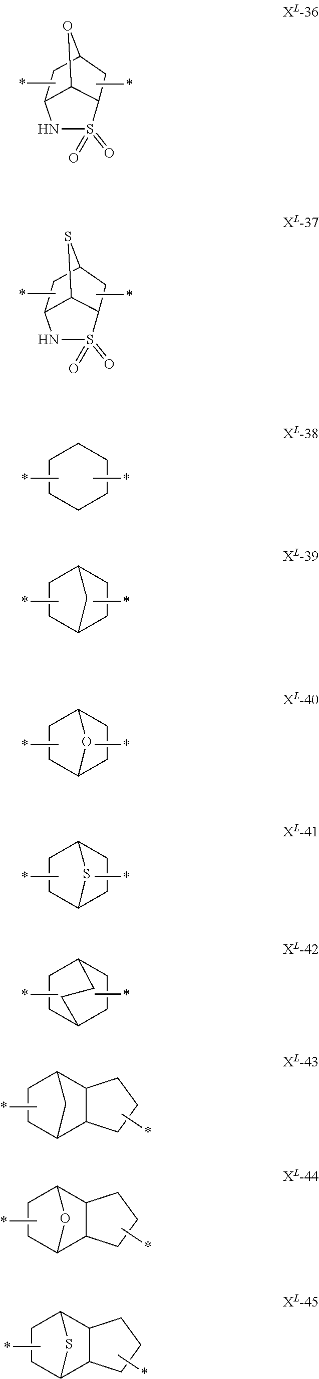

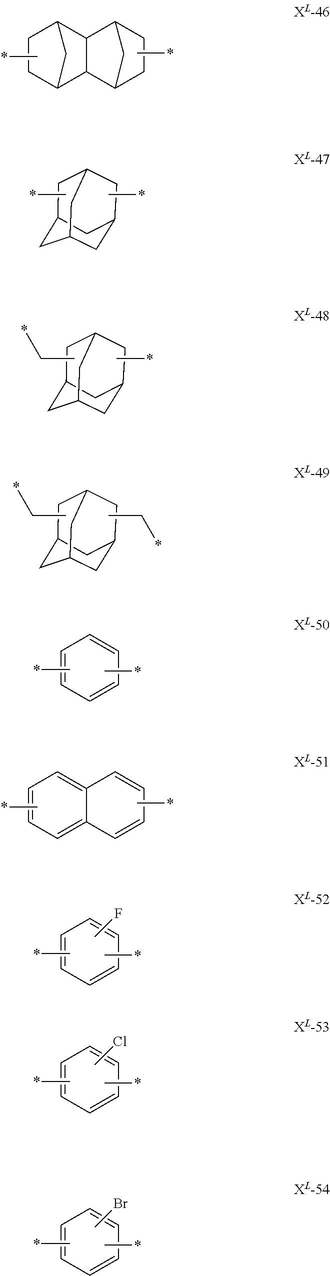

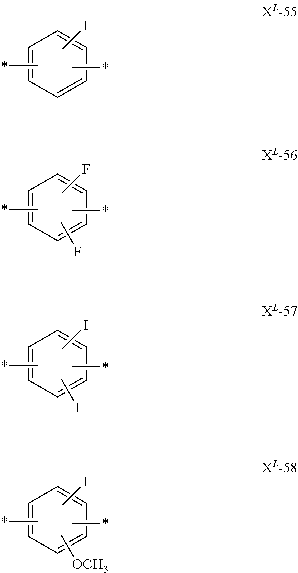

Of these, X^{L}-0 to X^{L}-22, X^{L}-29 to X^{L}-34, and X^{L}-47 to X¹-58 are preferable.

Provided that, in formula (Z), m13 and m14 are not 0 at the same time, and L^{A}, L^{B}, L^{C}, L^{D}, X^{L1}, and X^{L2} are not a single bond at the same time.

Specific examples of the anion represented by Z⁻ are shown below, but not limited thereto. In the following formula. R^{A} is as defined above, and Me is a methyl group. The bonding positions of various substituents on the aromatic ring may be interchanged with each other.

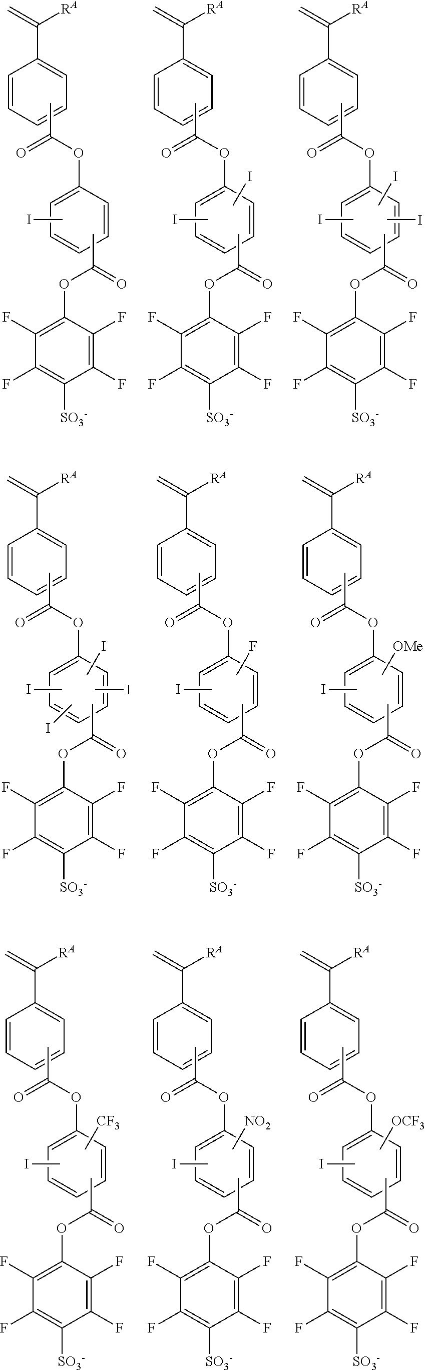

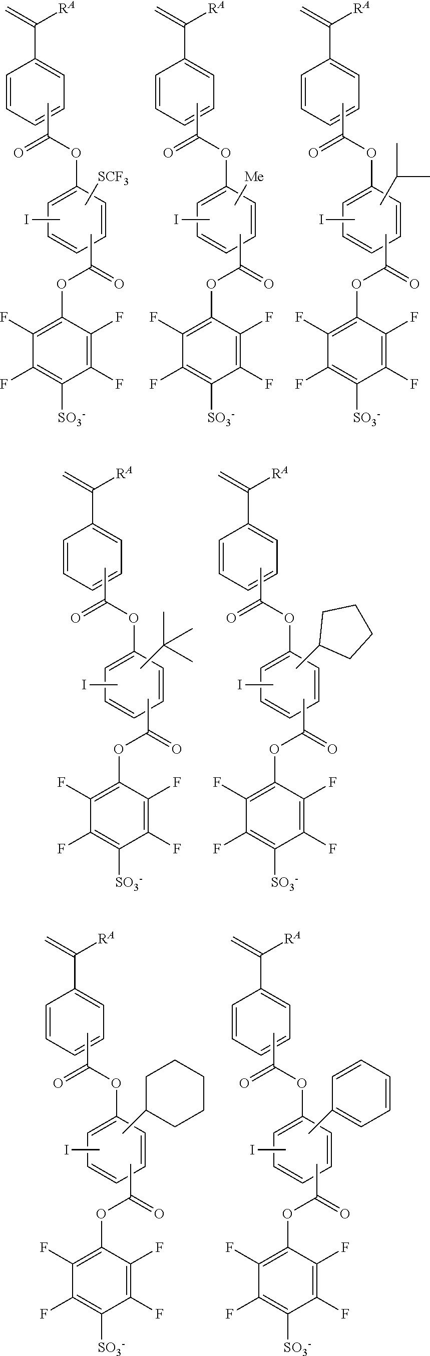

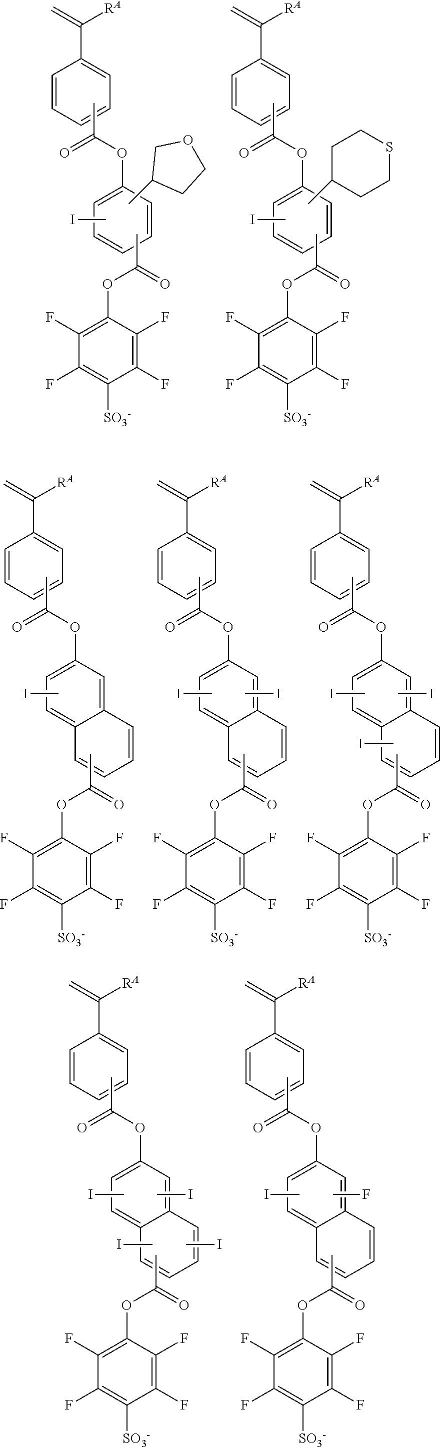

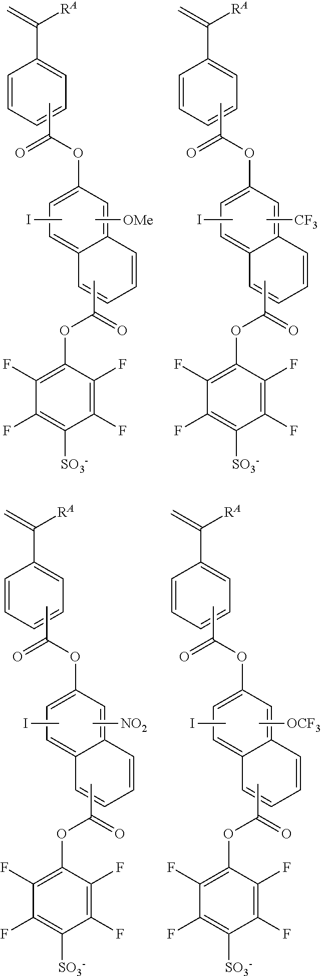

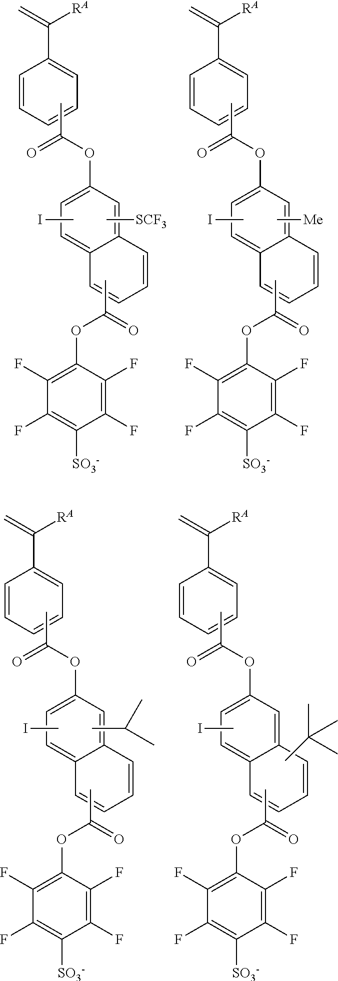

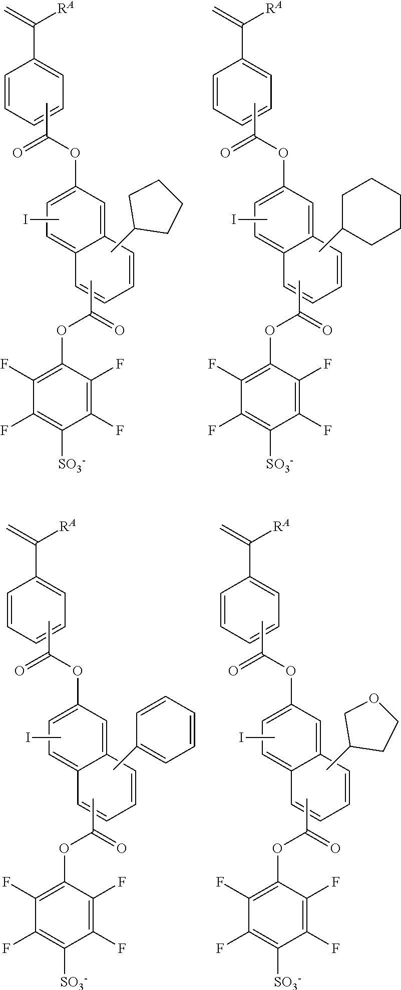

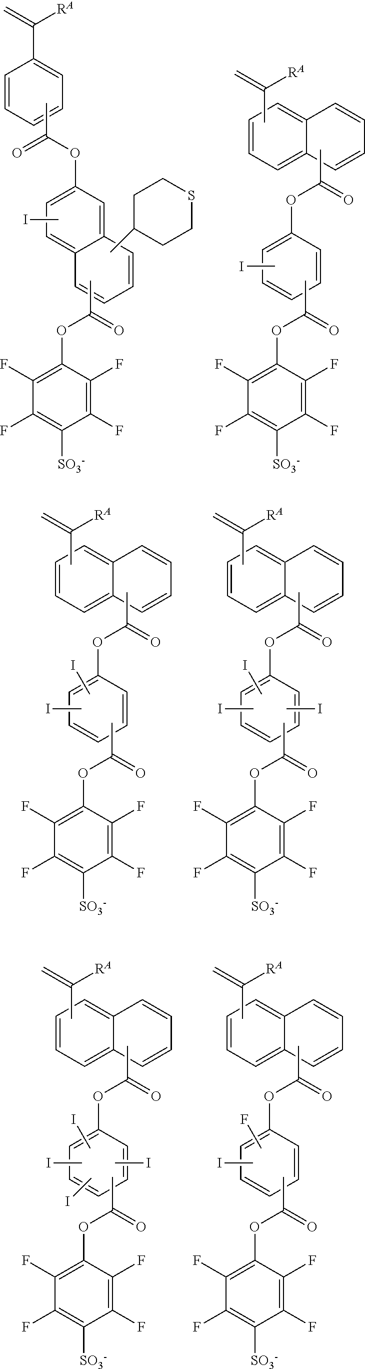

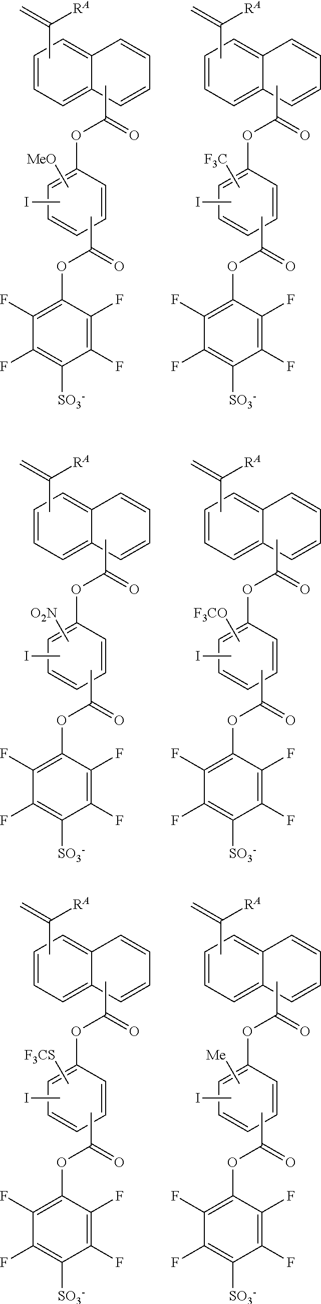

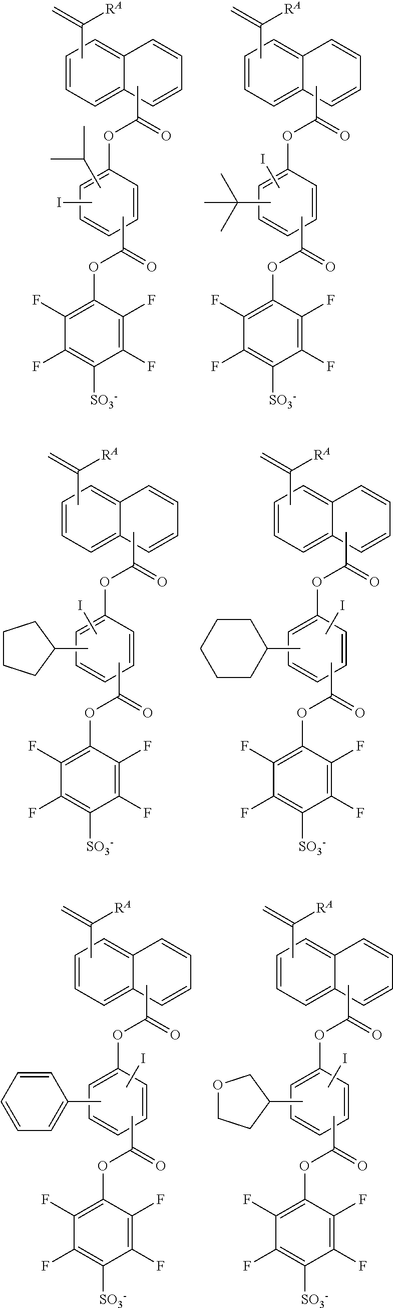

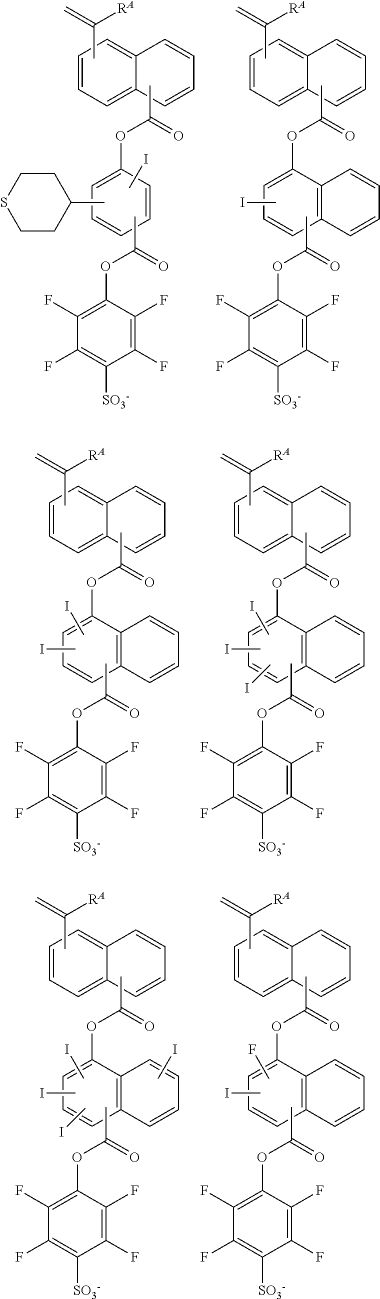

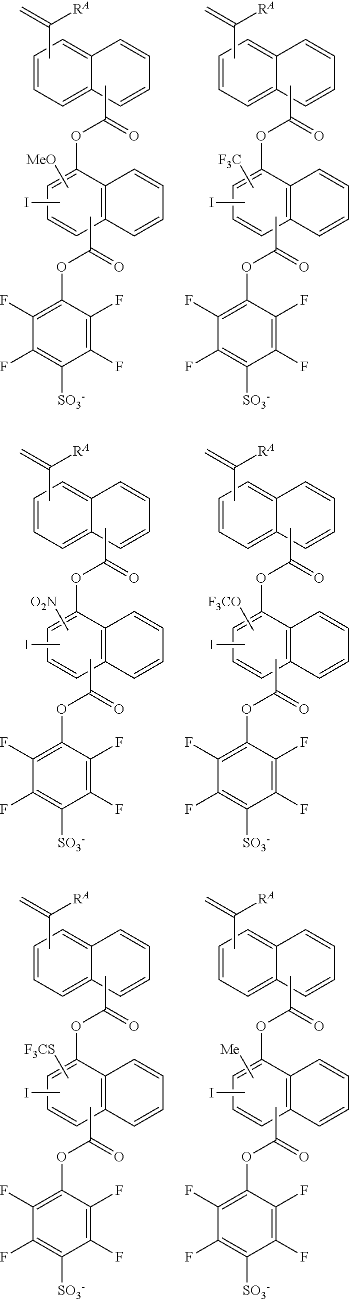

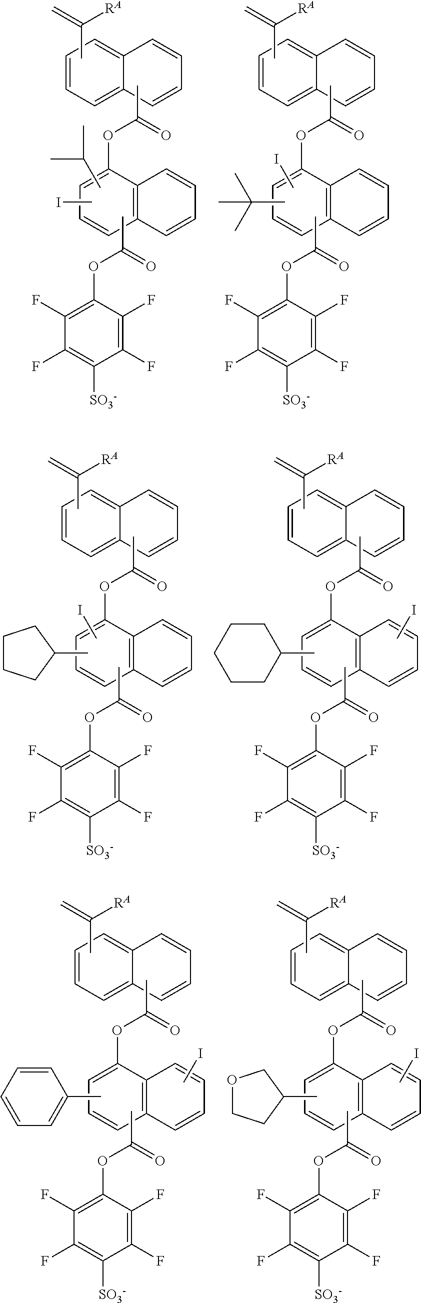

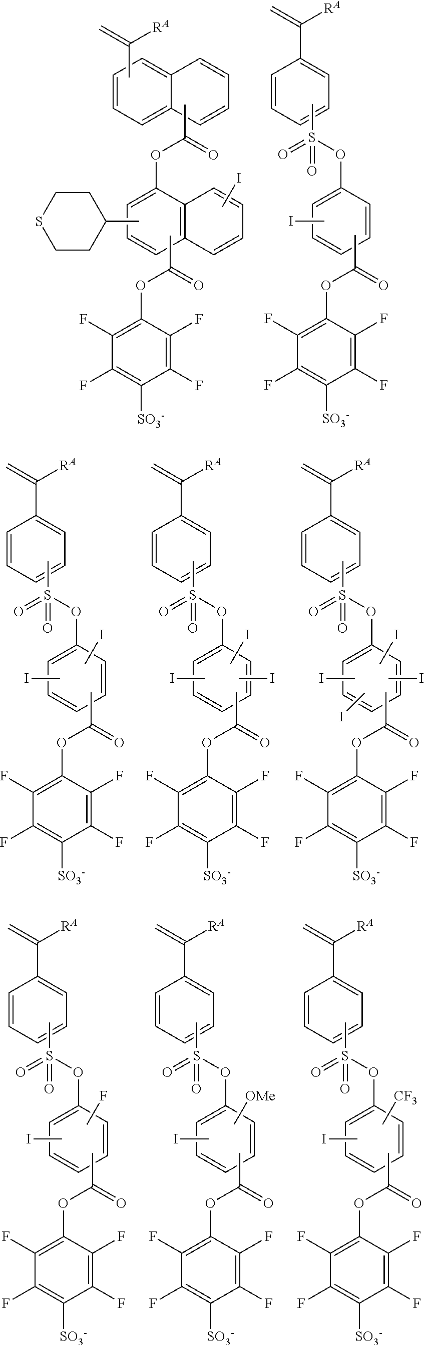

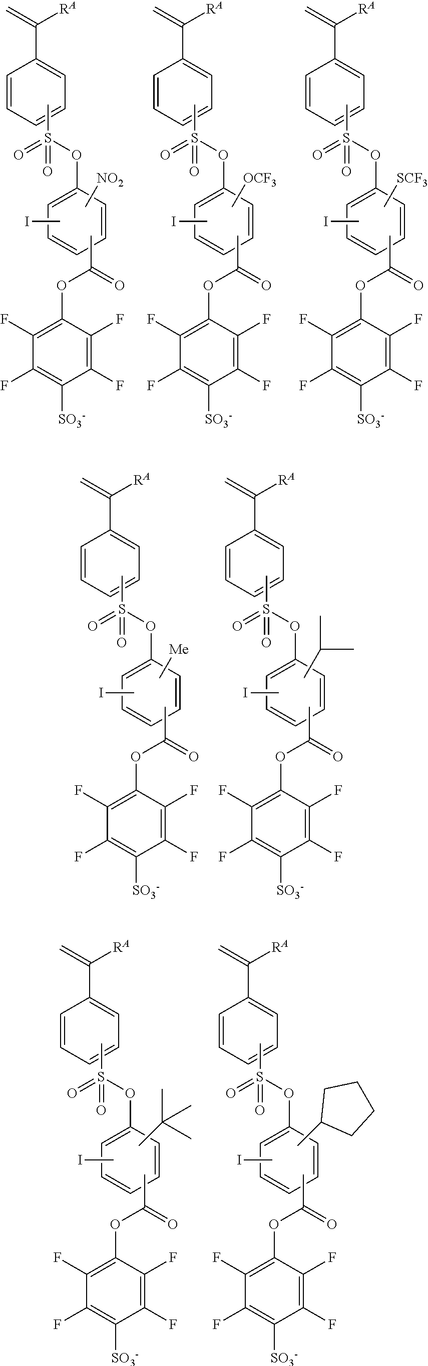

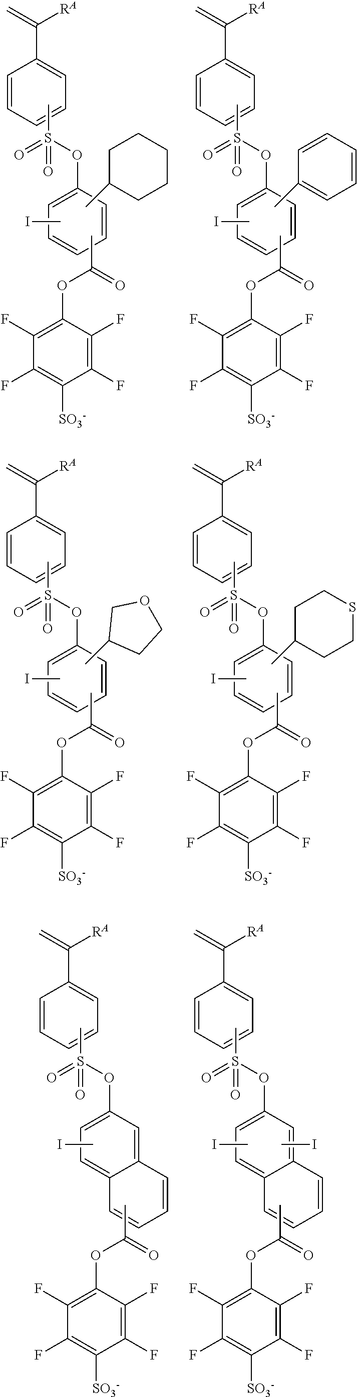

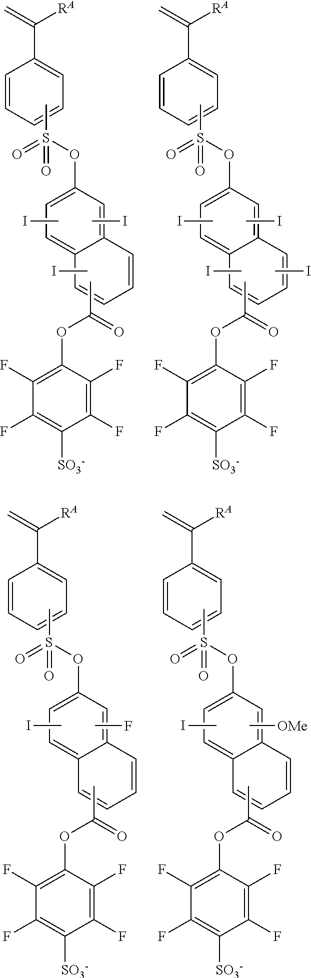

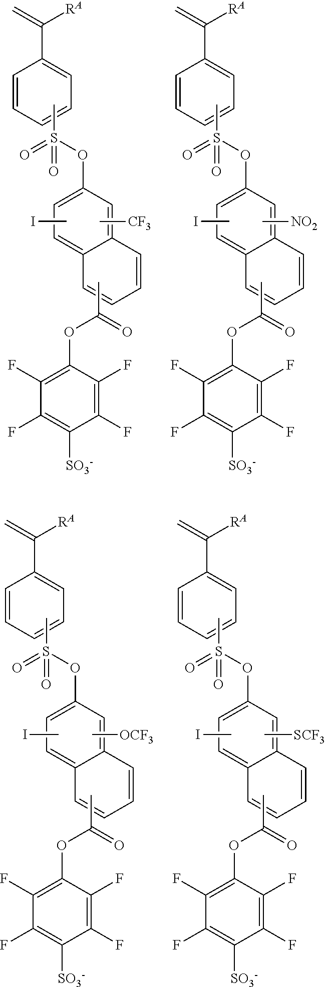

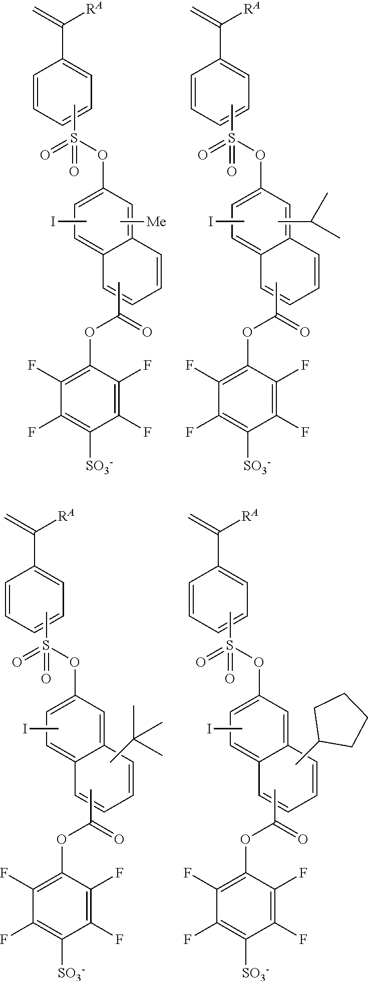

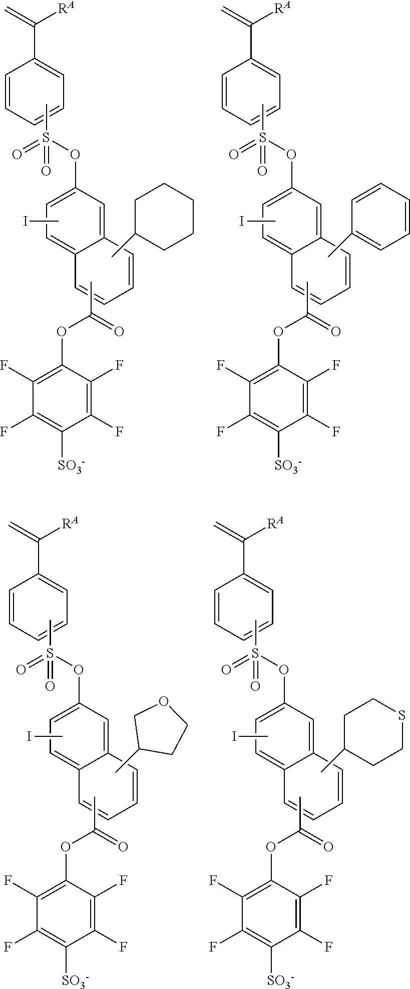

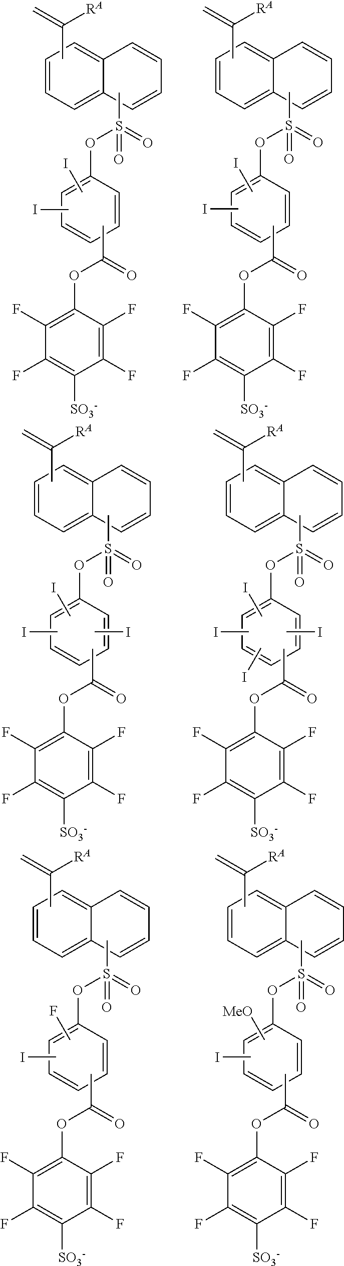

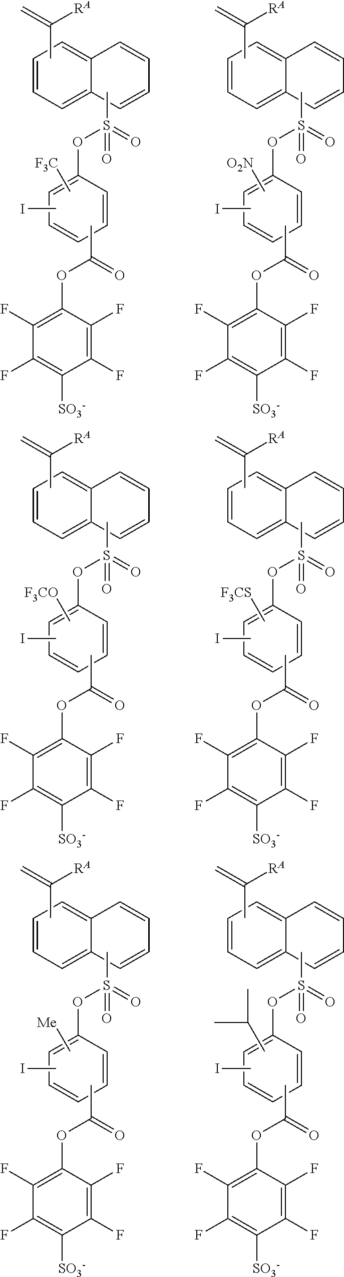

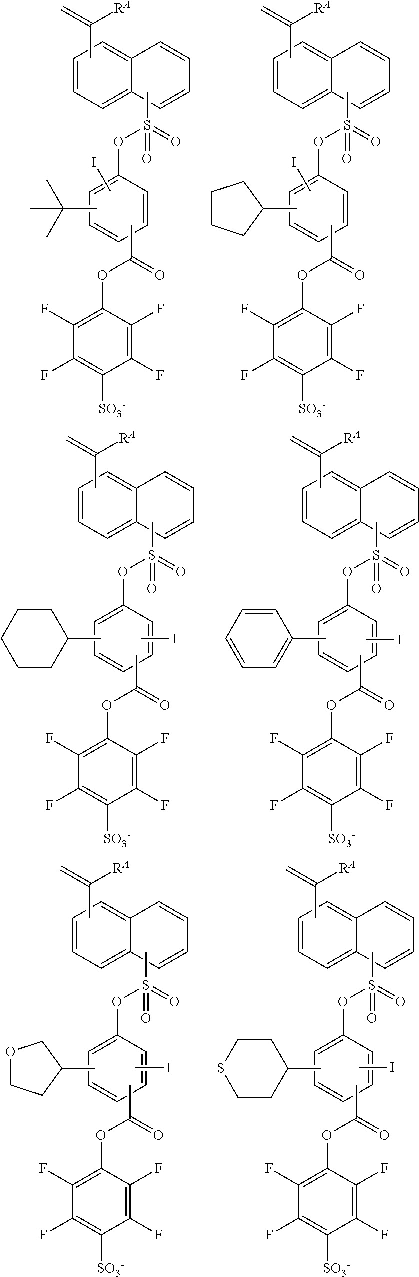

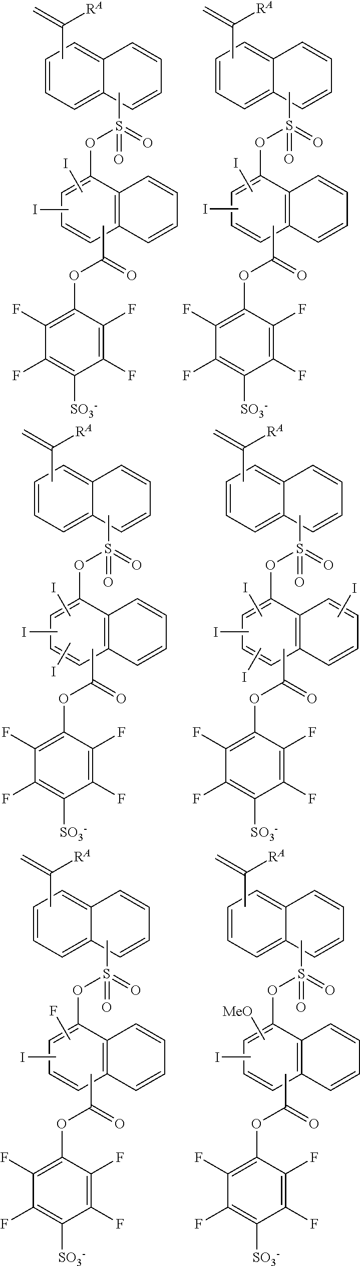

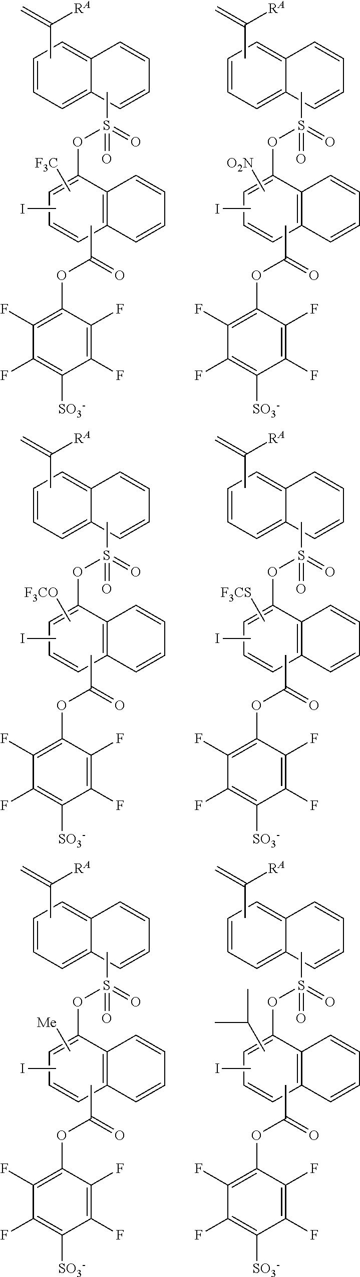

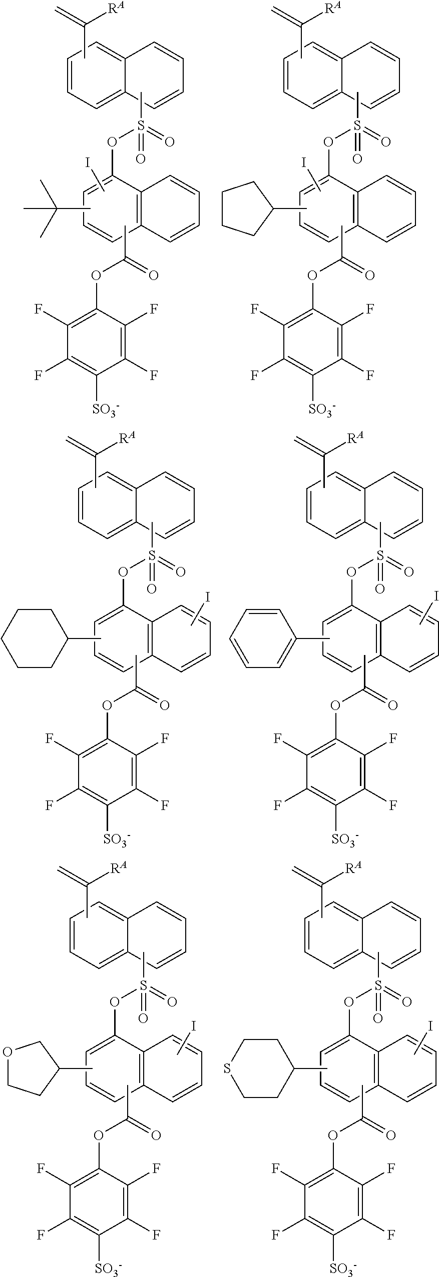

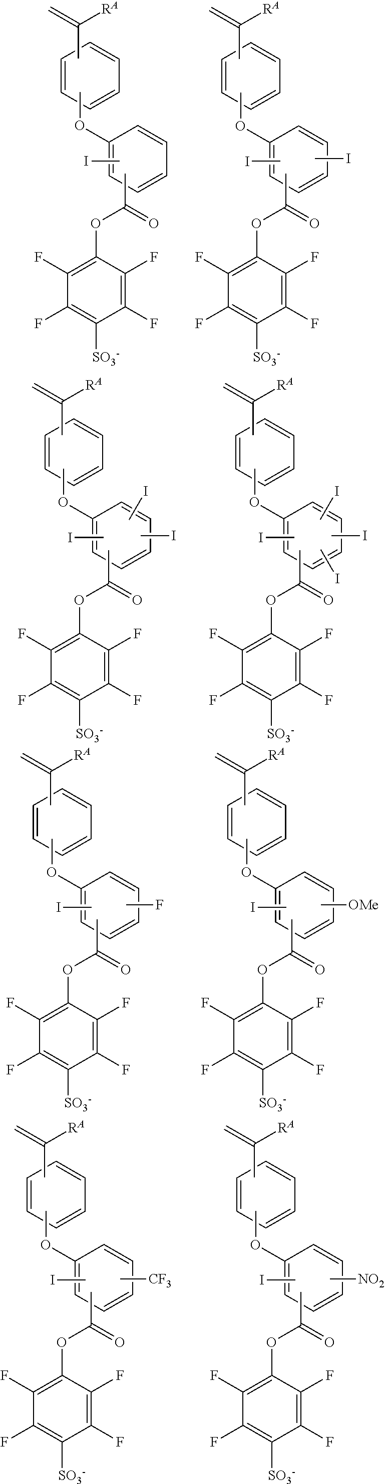

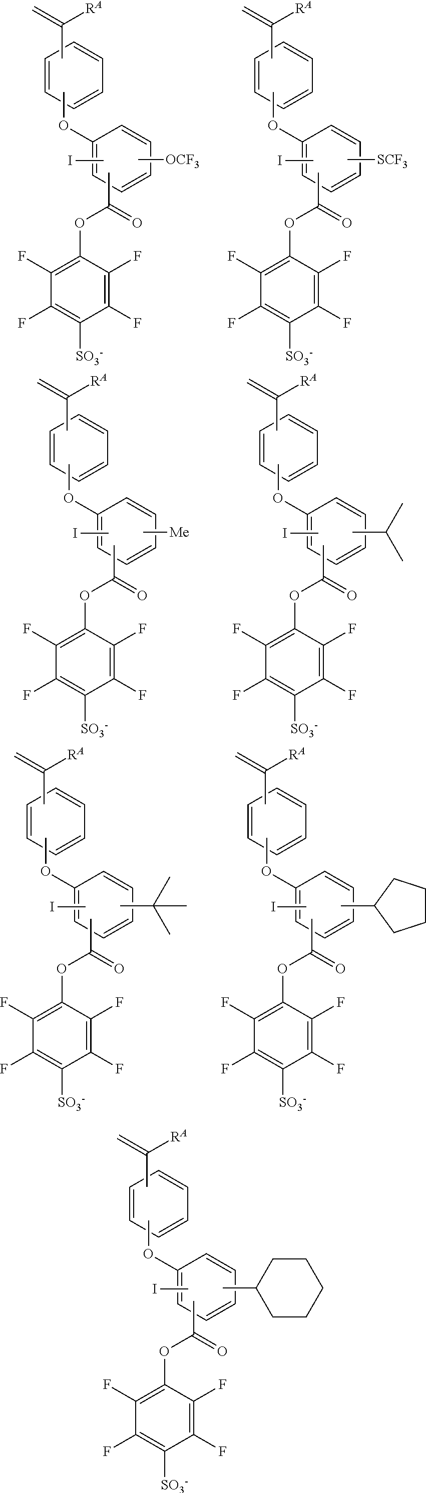

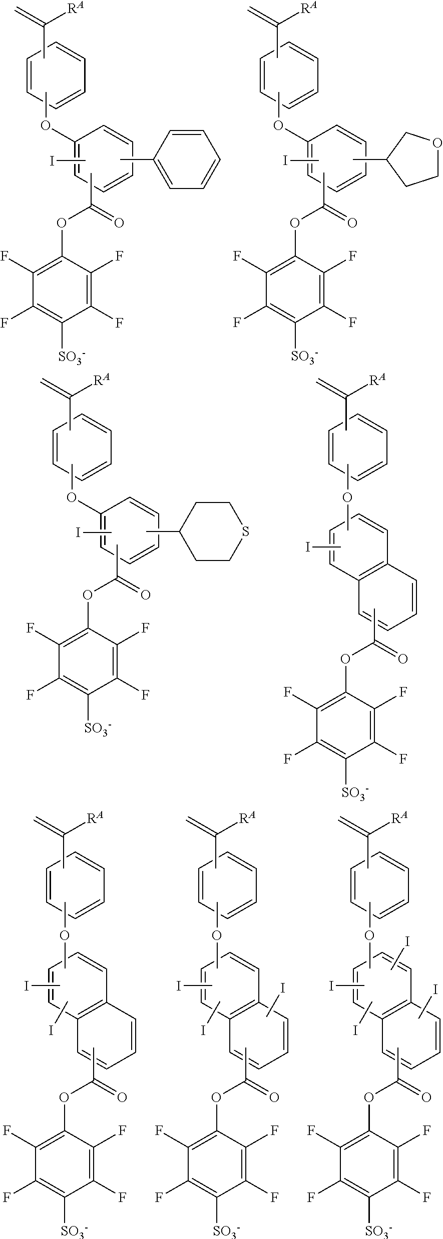

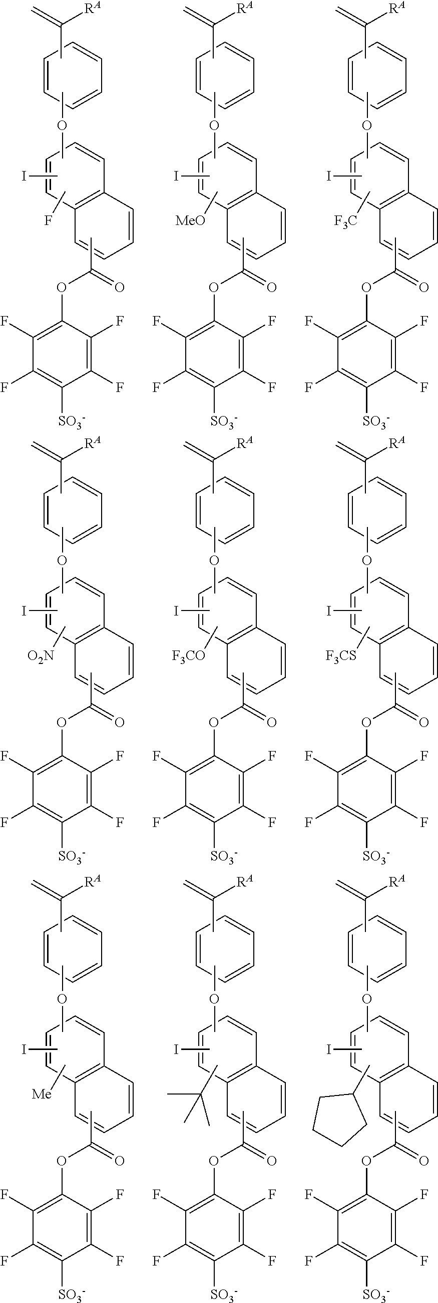

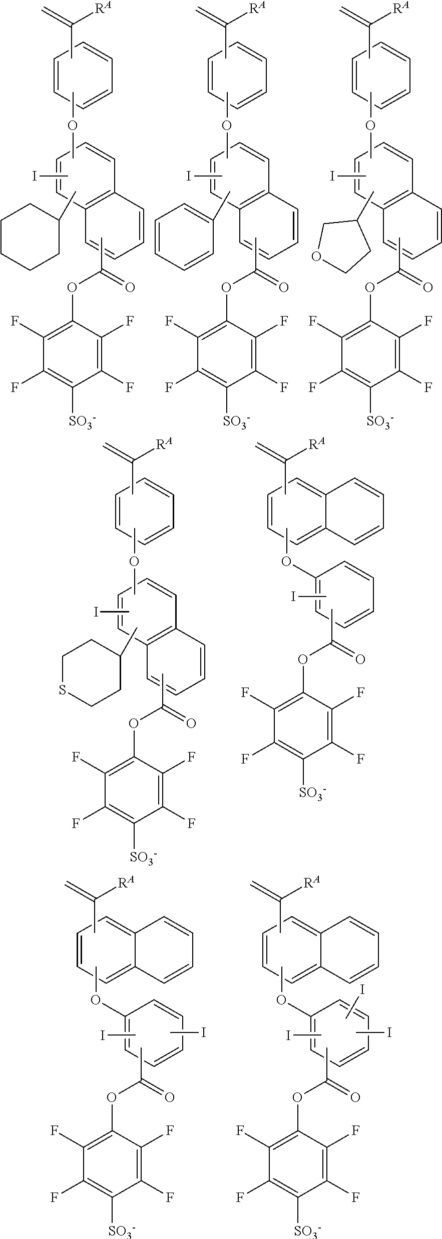

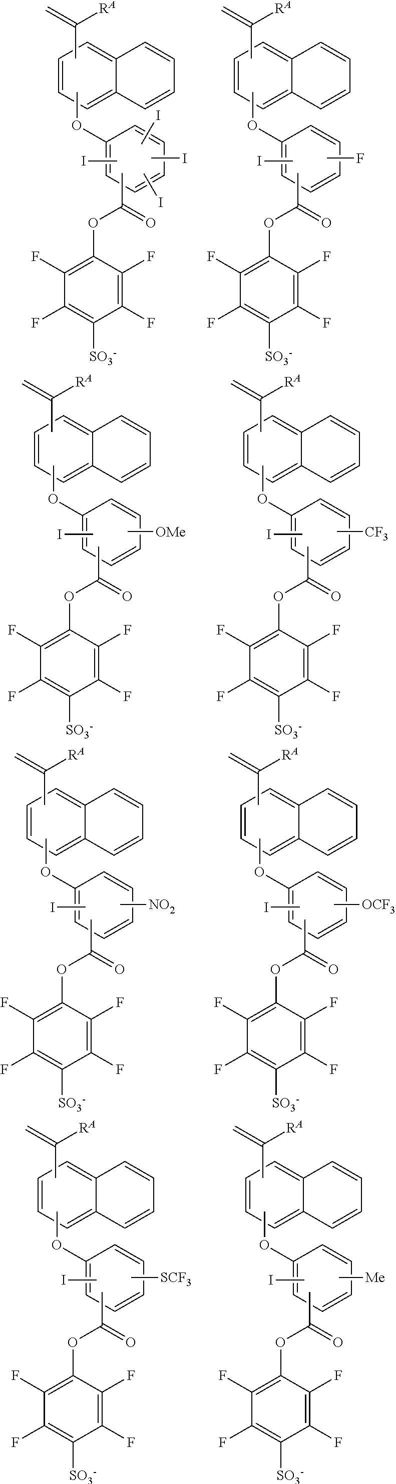

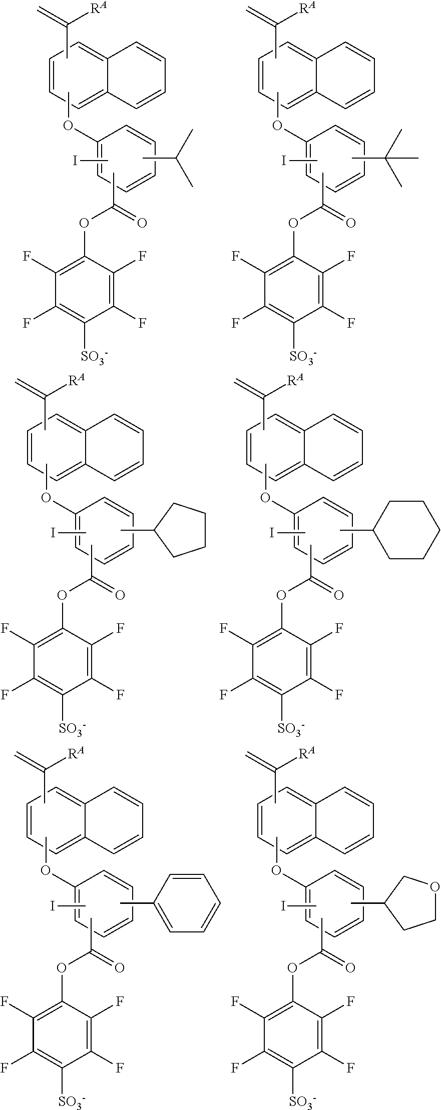

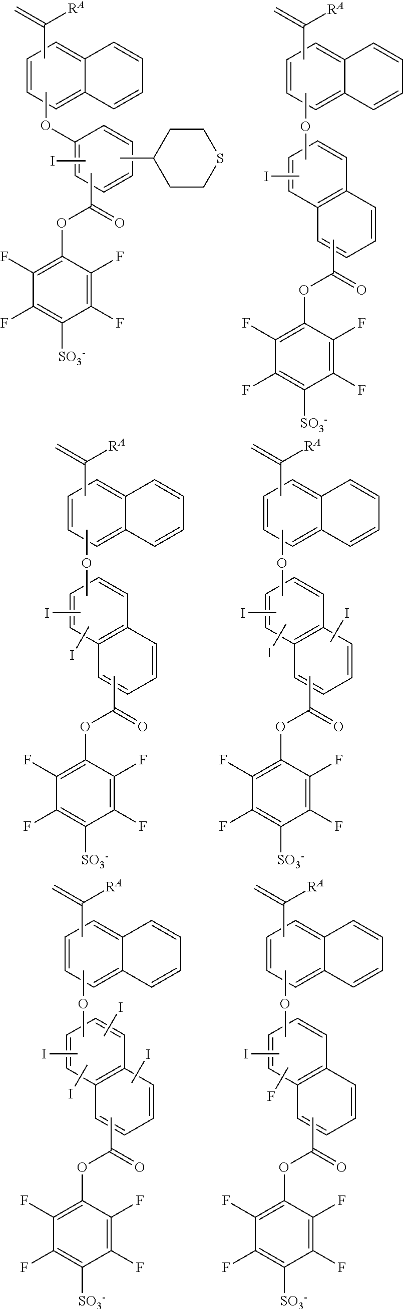

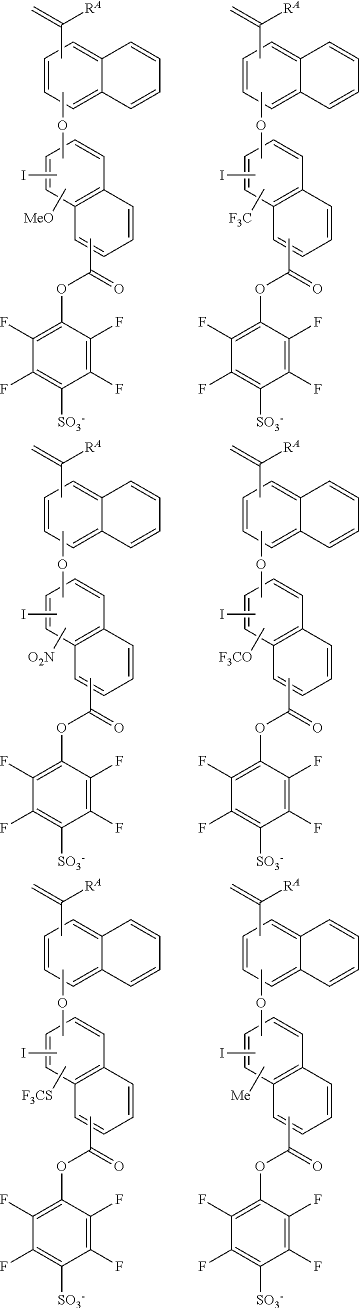

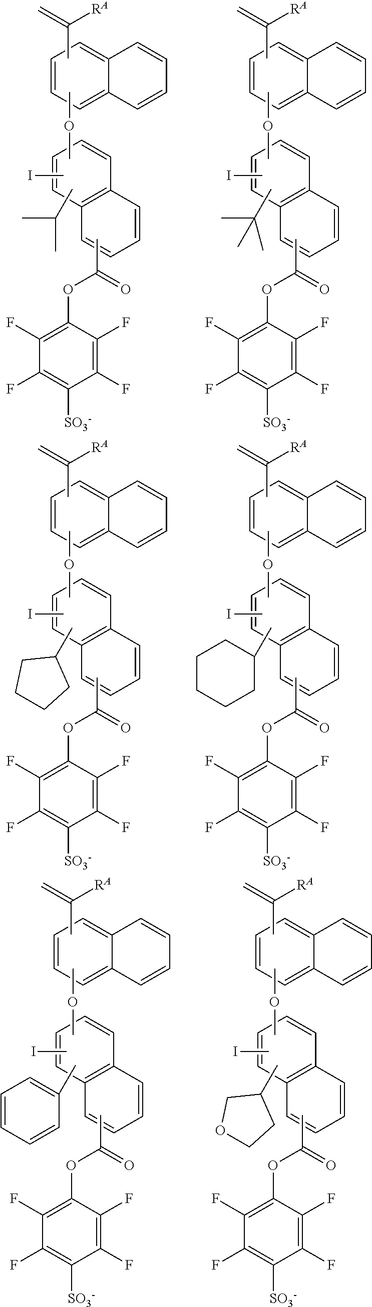

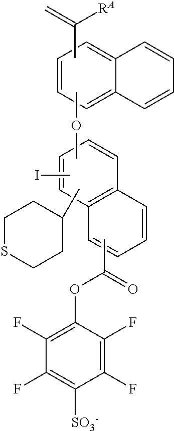

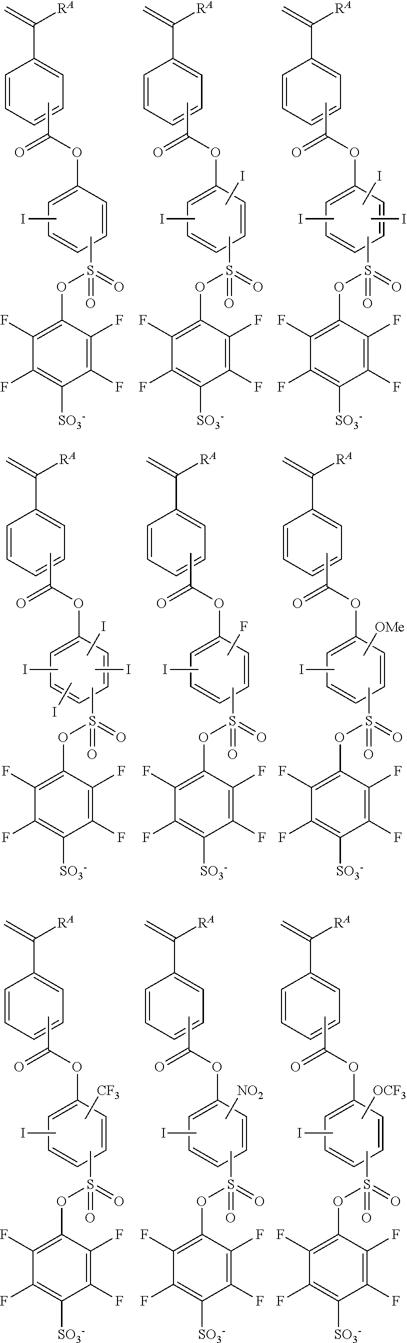

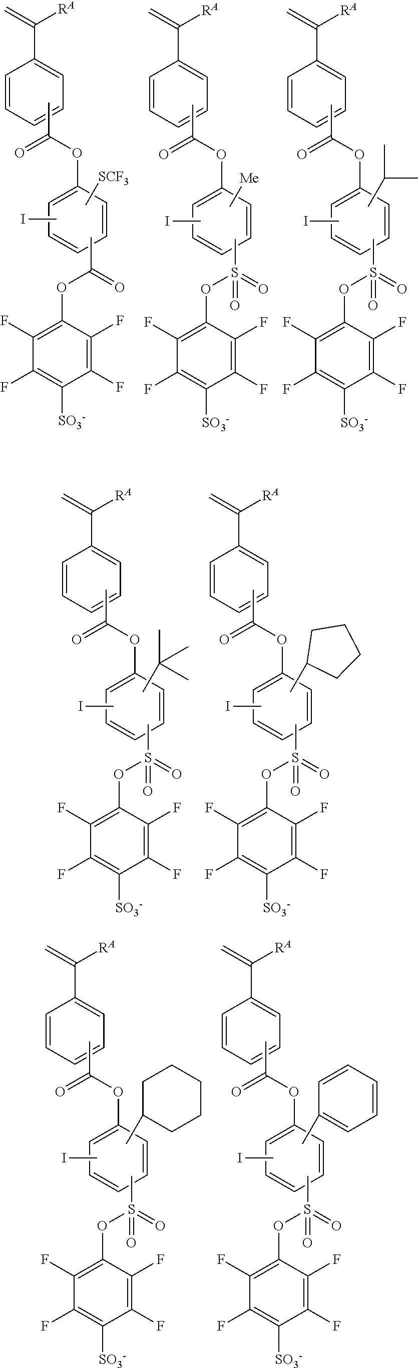

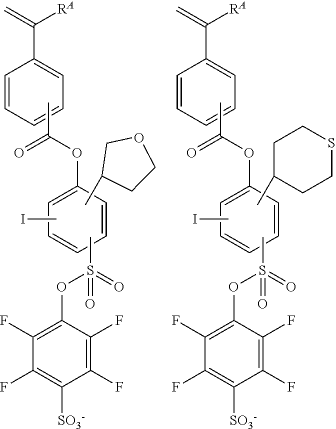

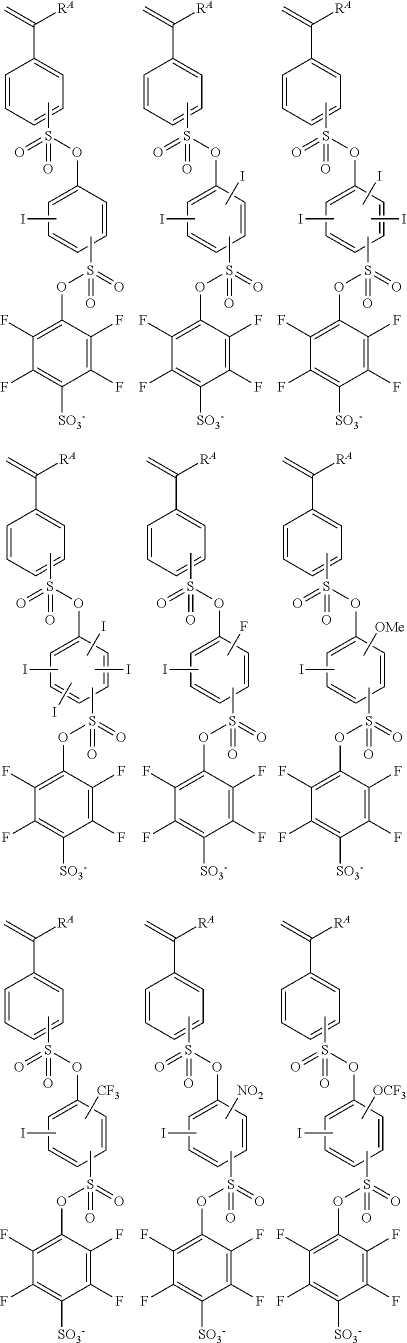

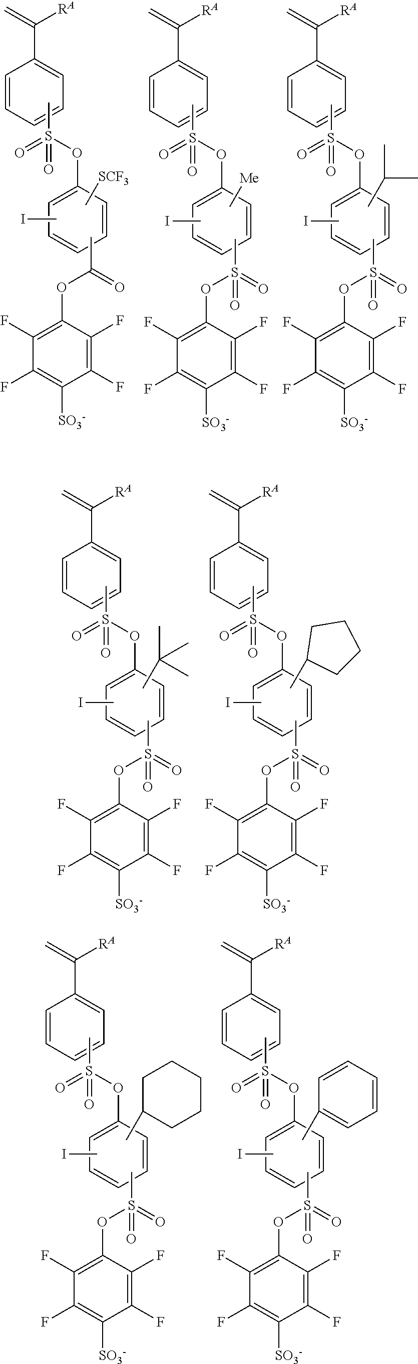

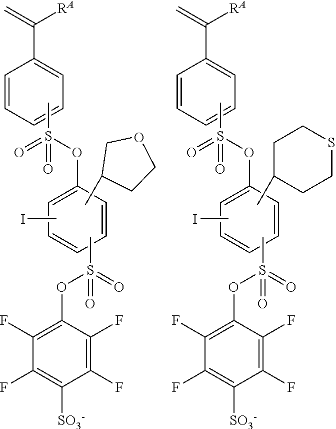

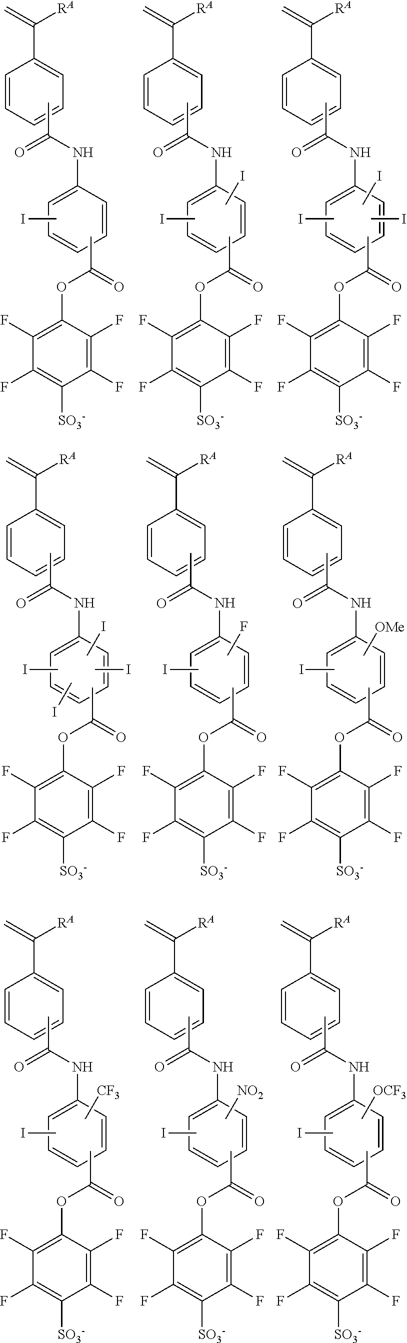

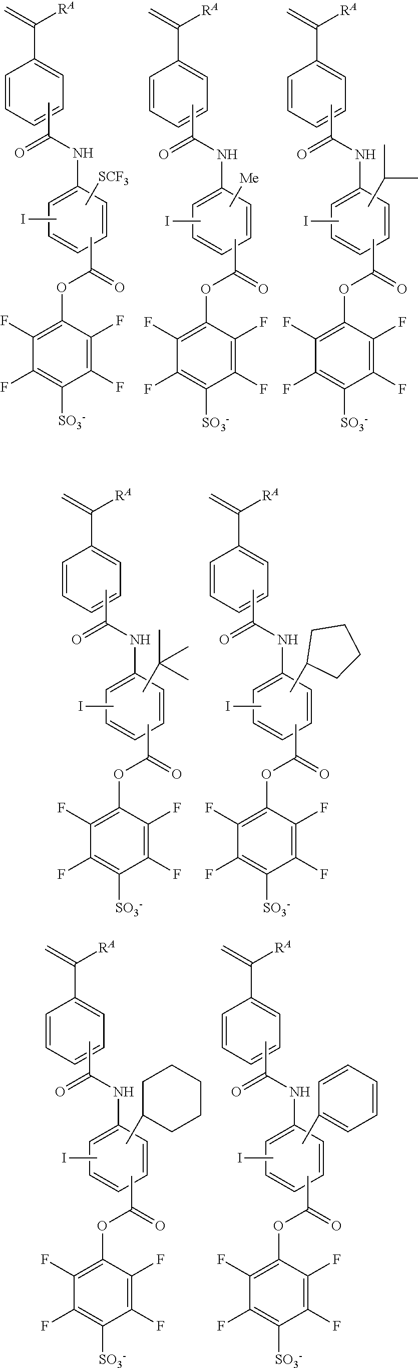

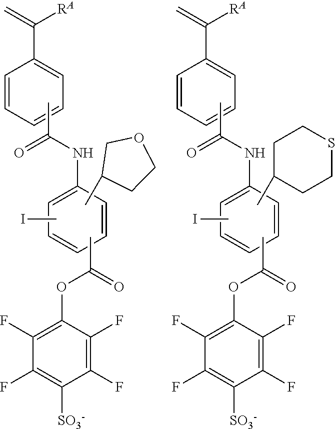

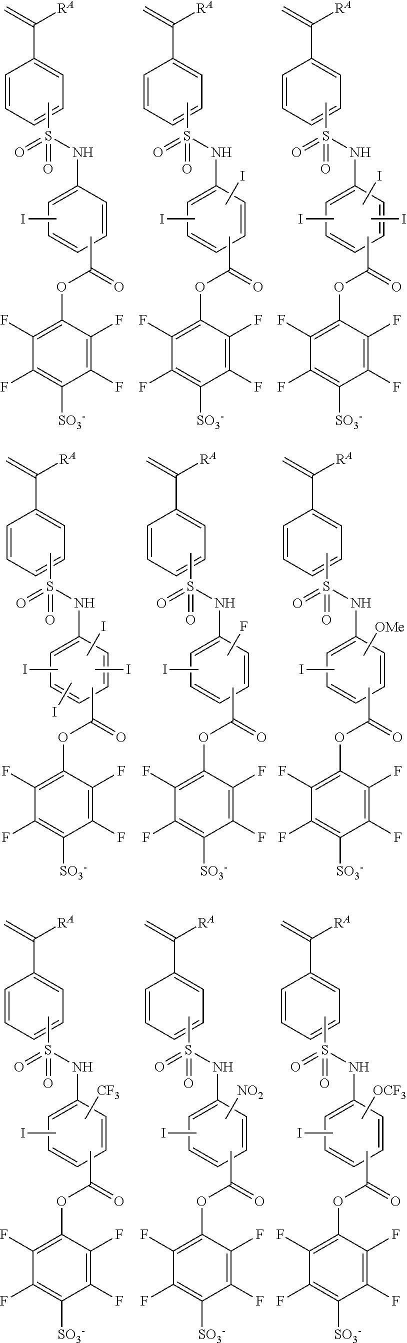

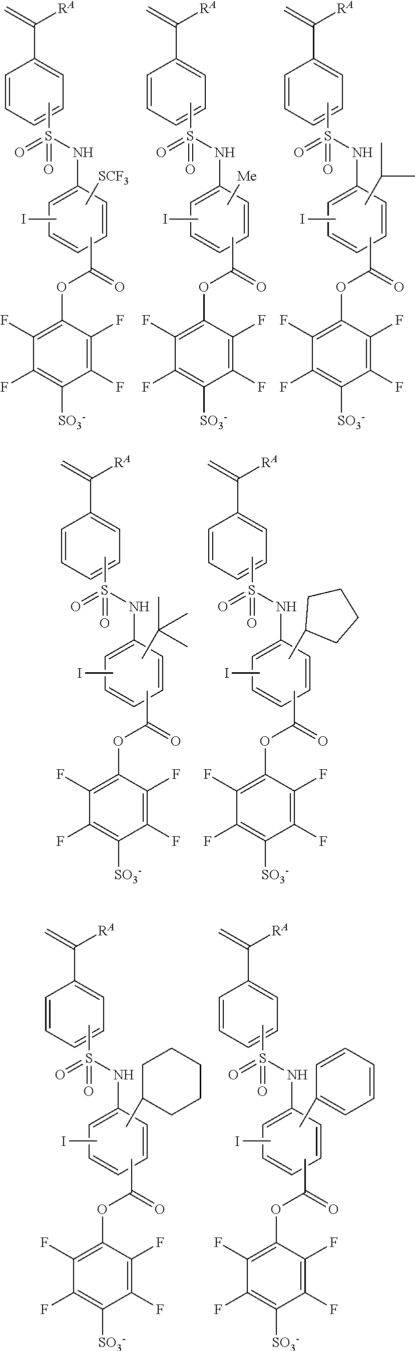

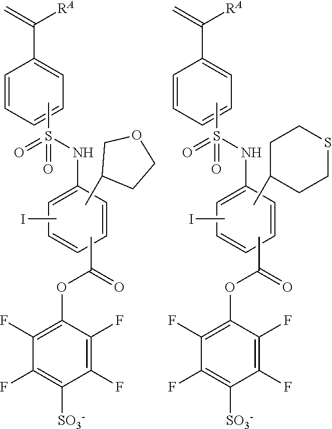

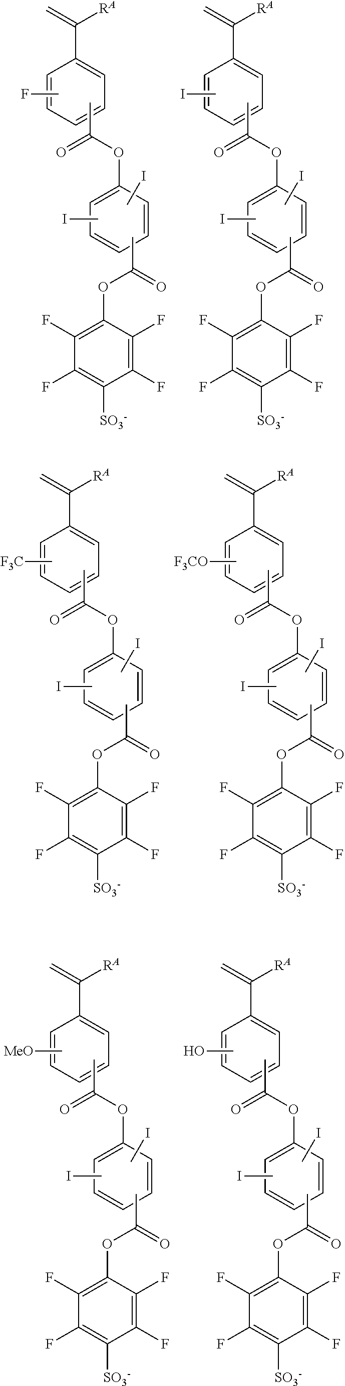

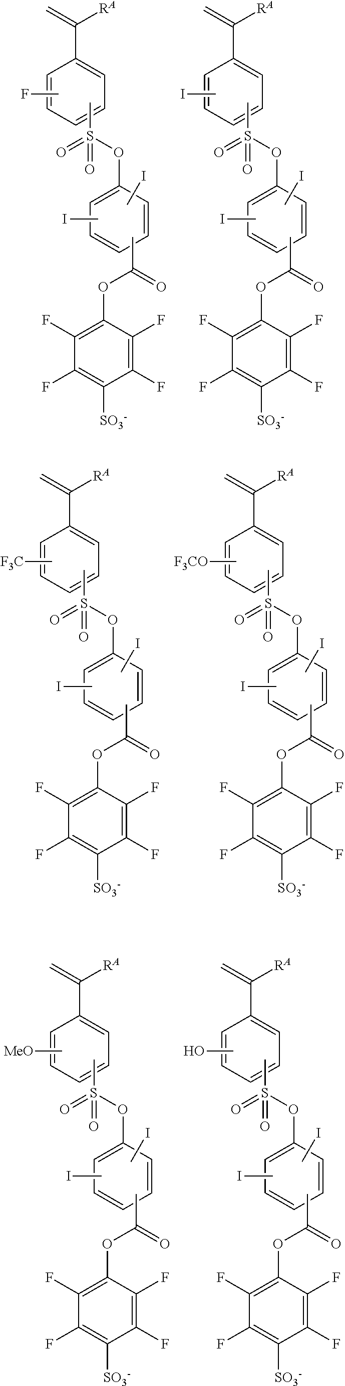

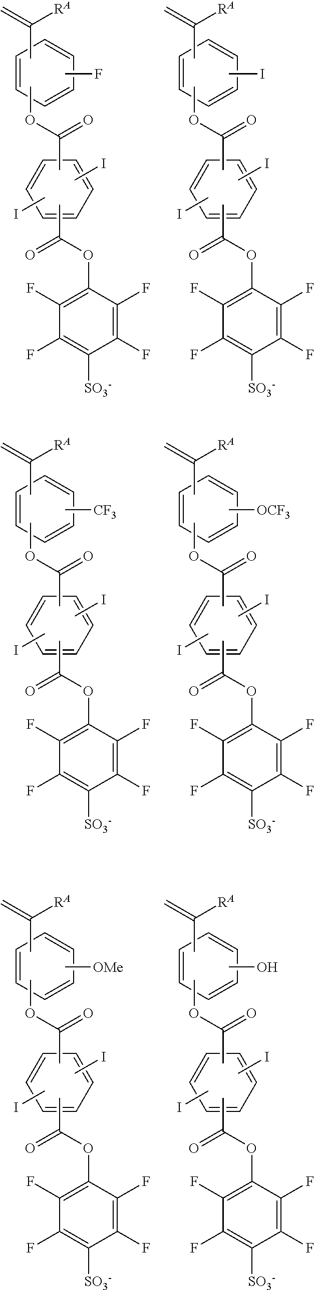

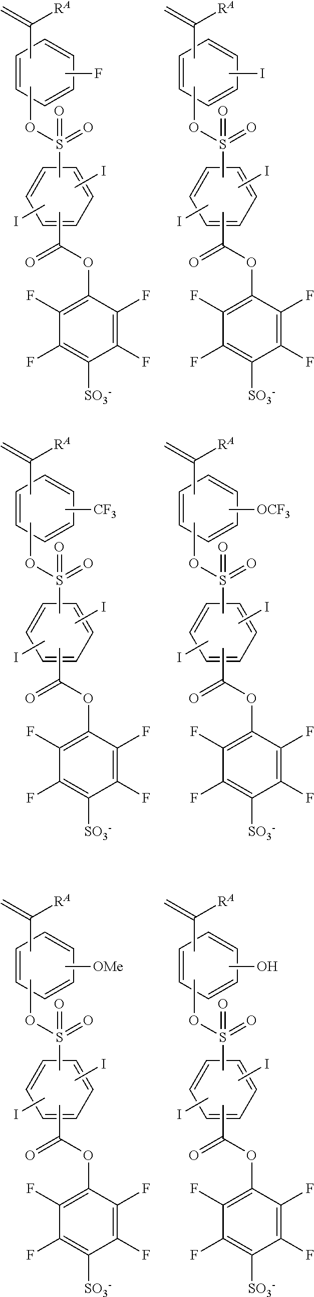

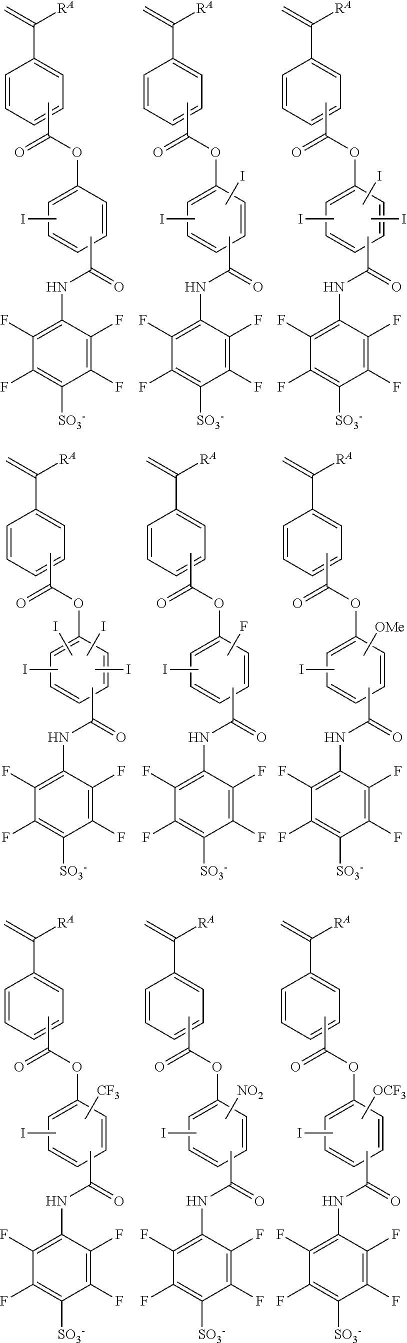

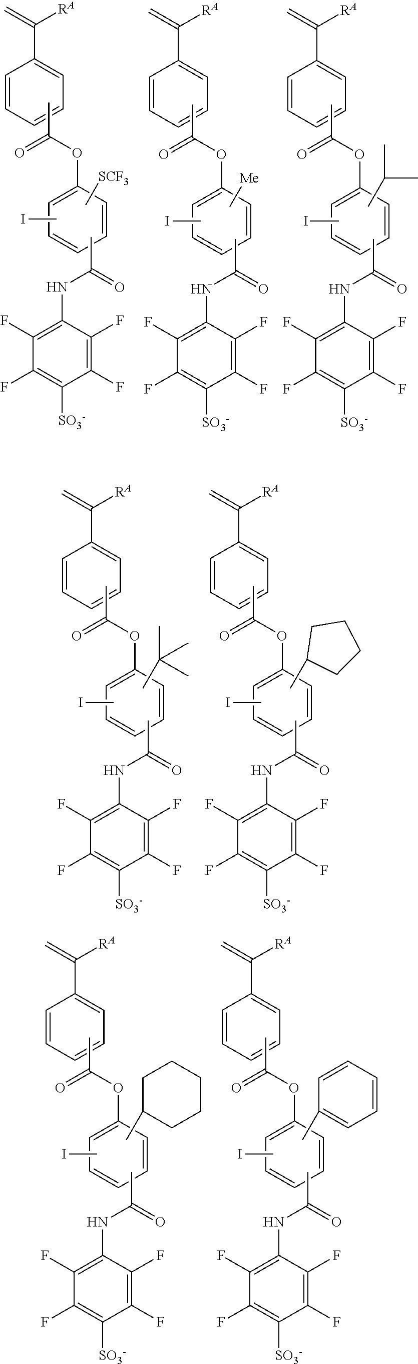

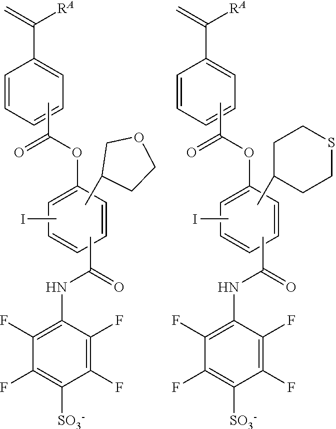

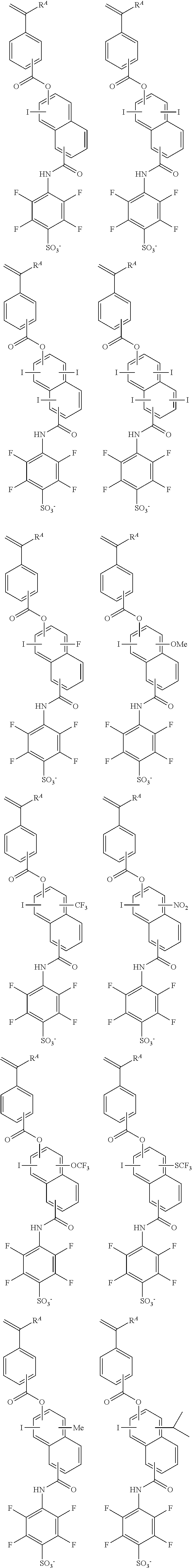

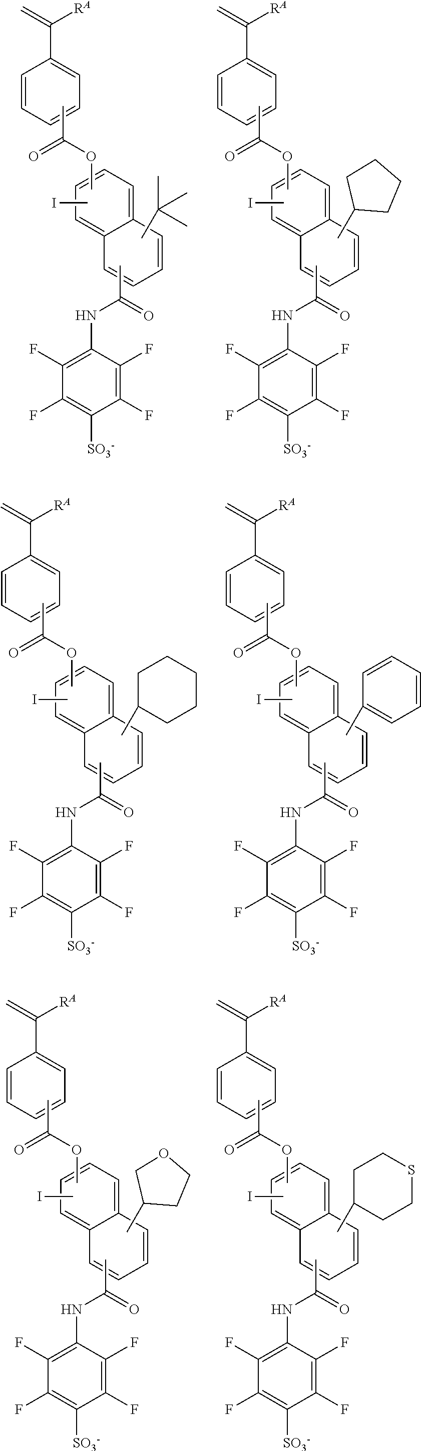

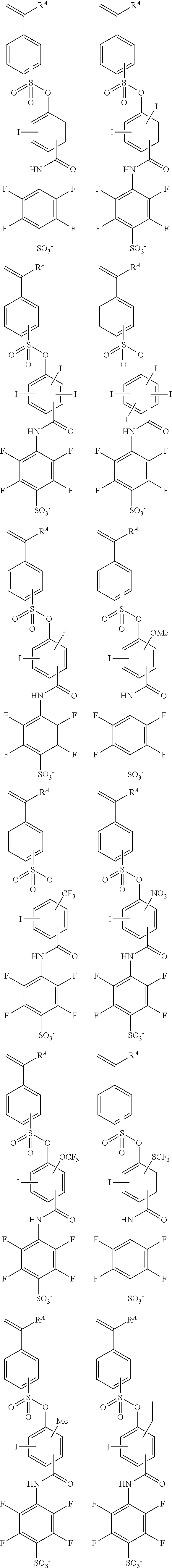

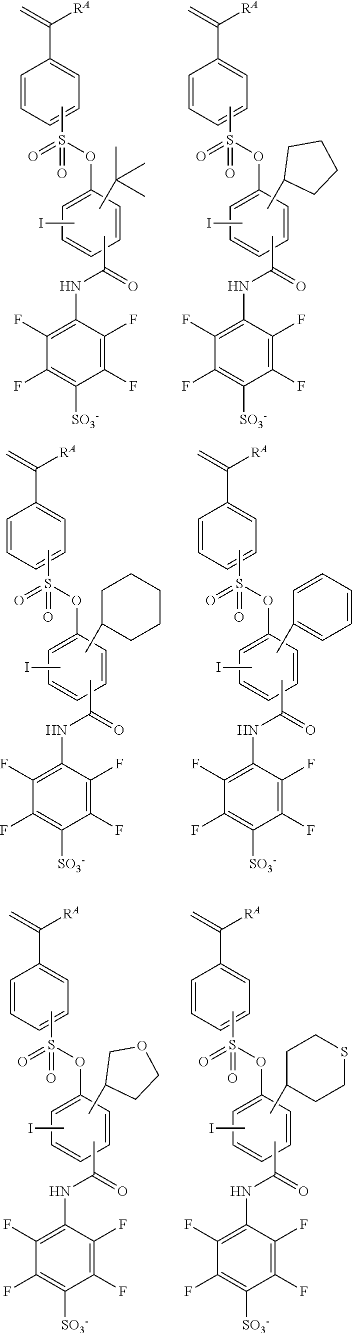

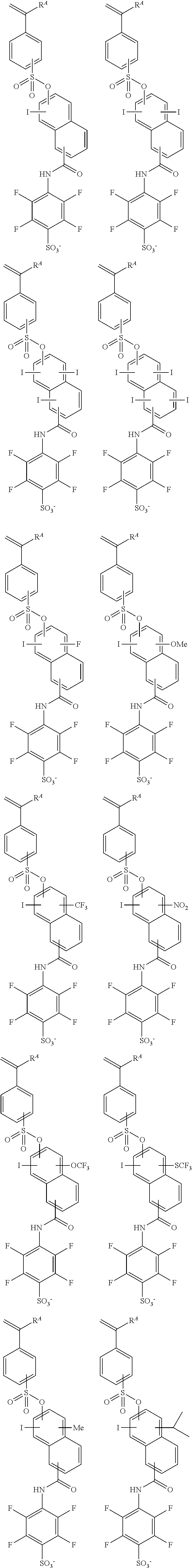

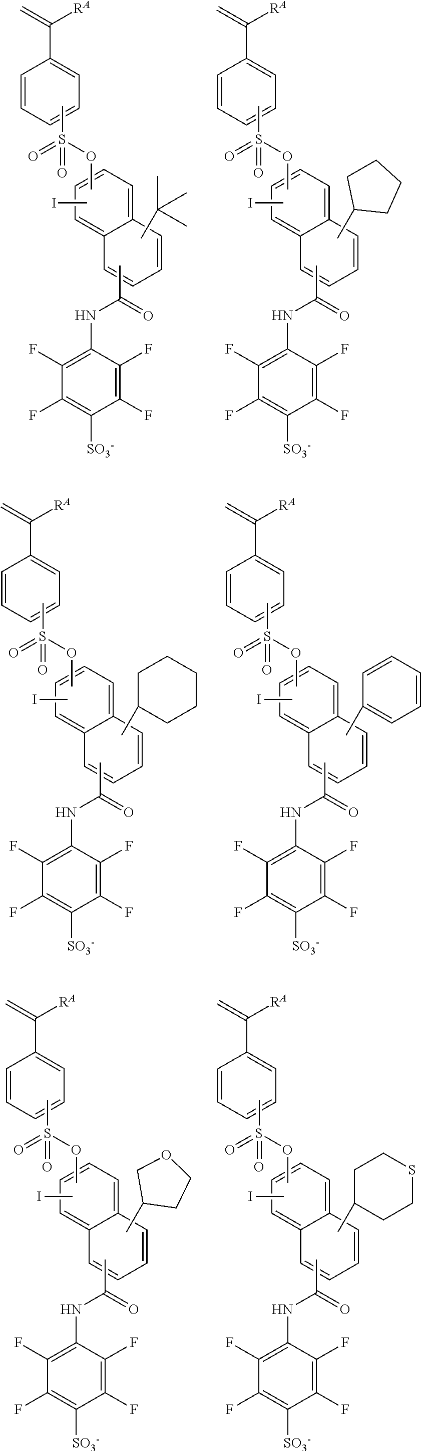

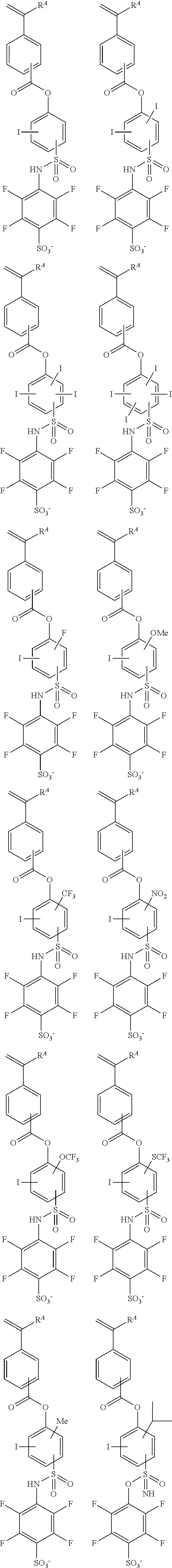

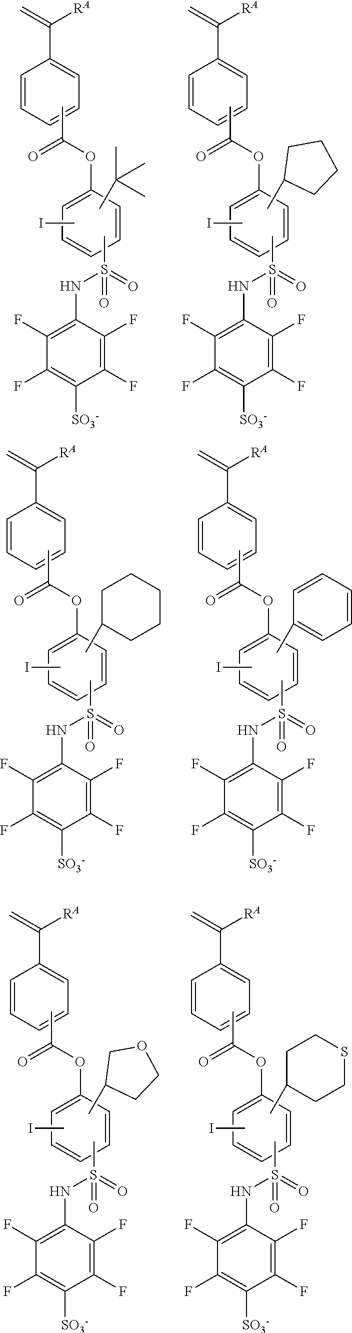

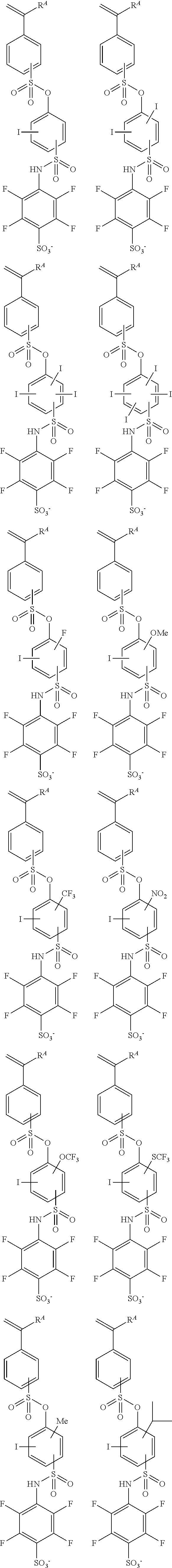

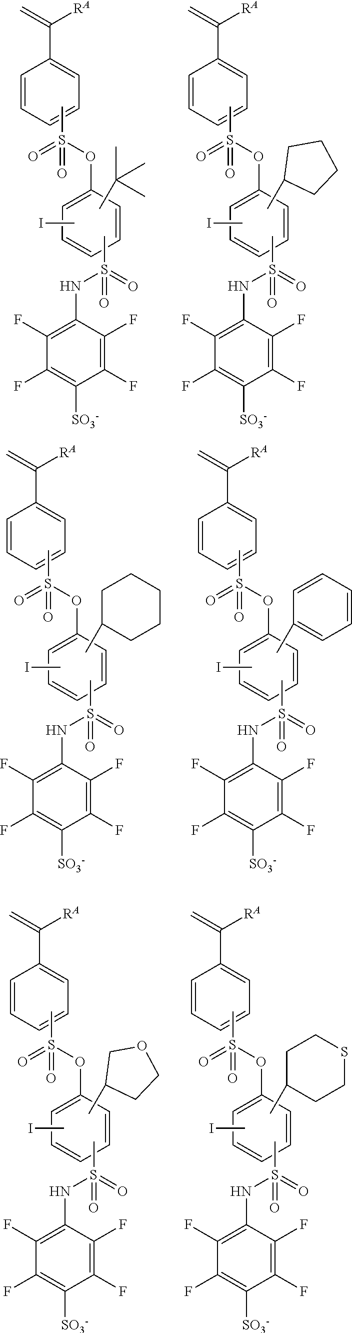

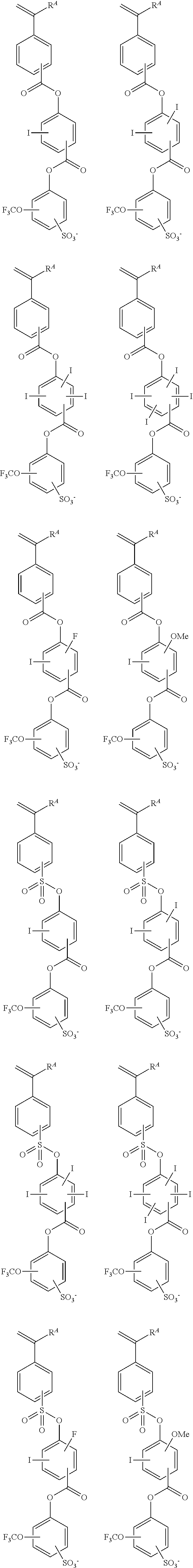

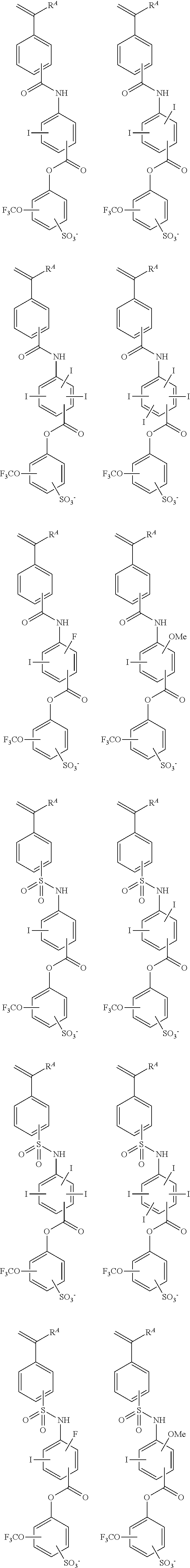

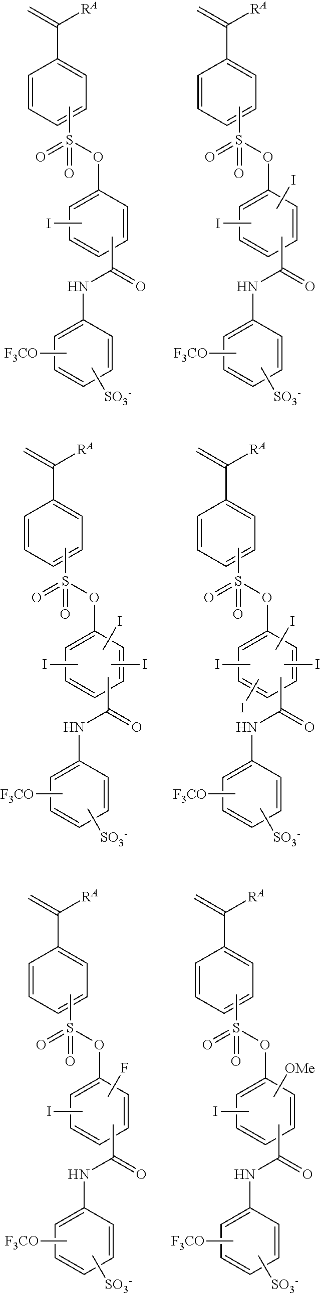

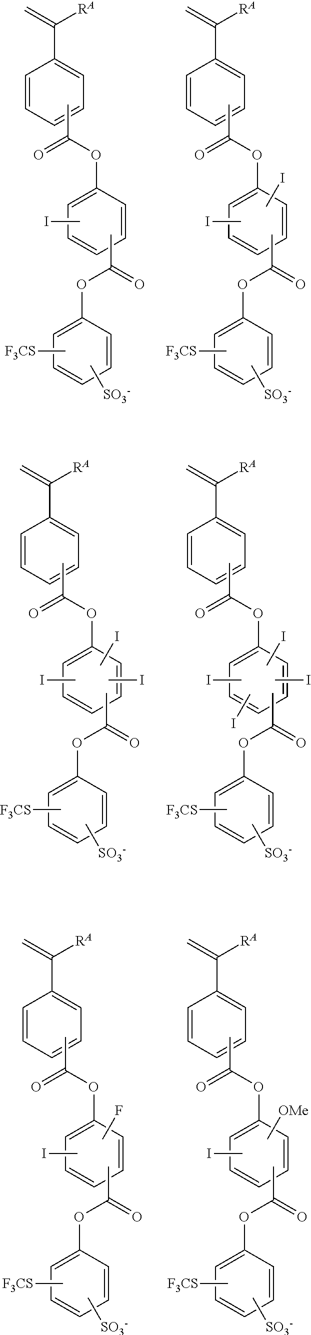

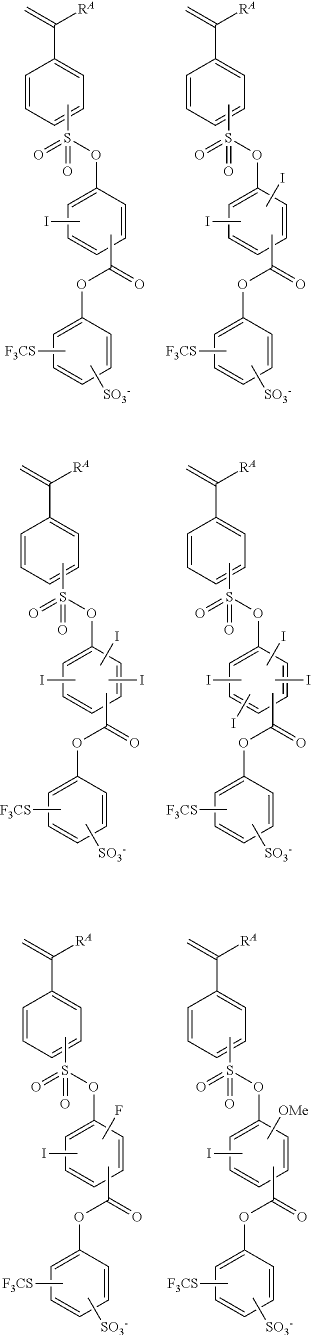

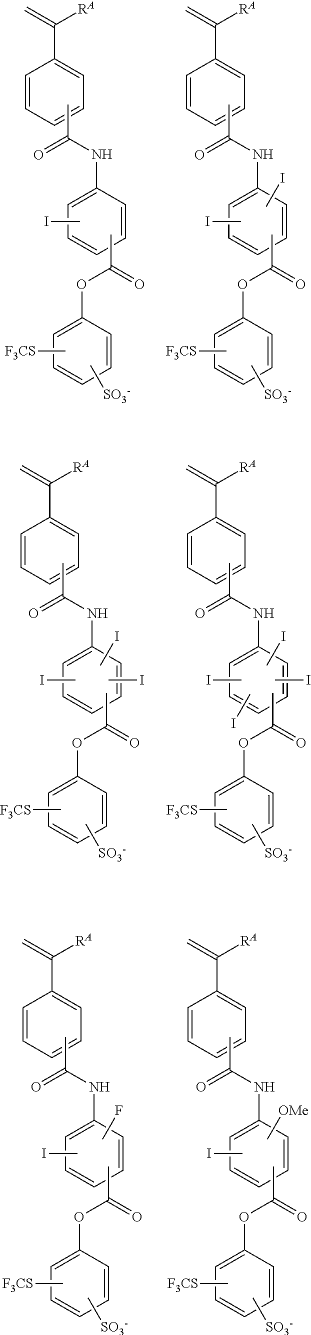

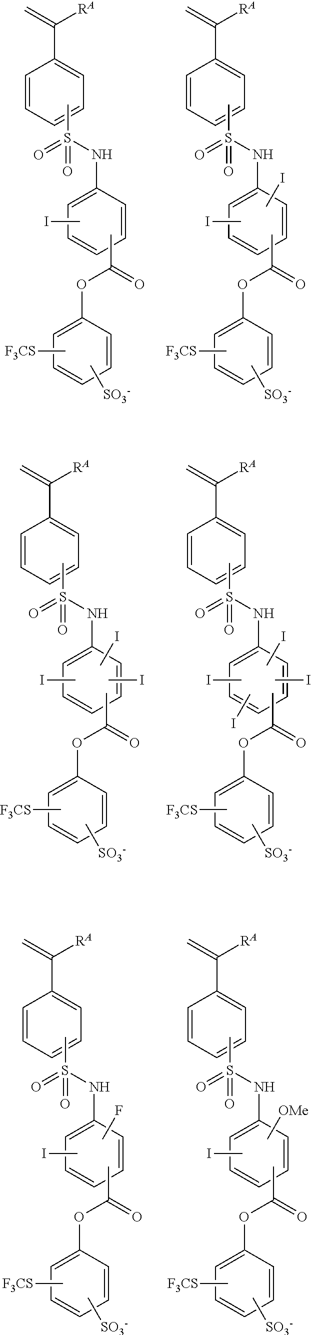

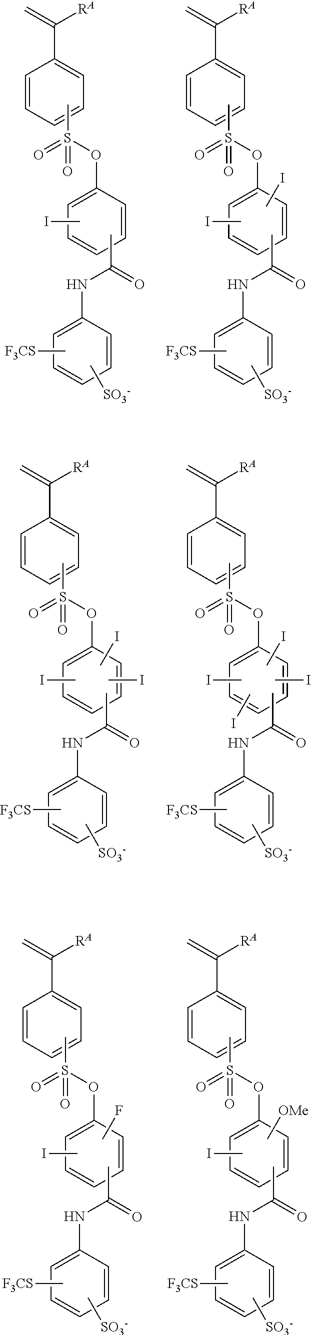

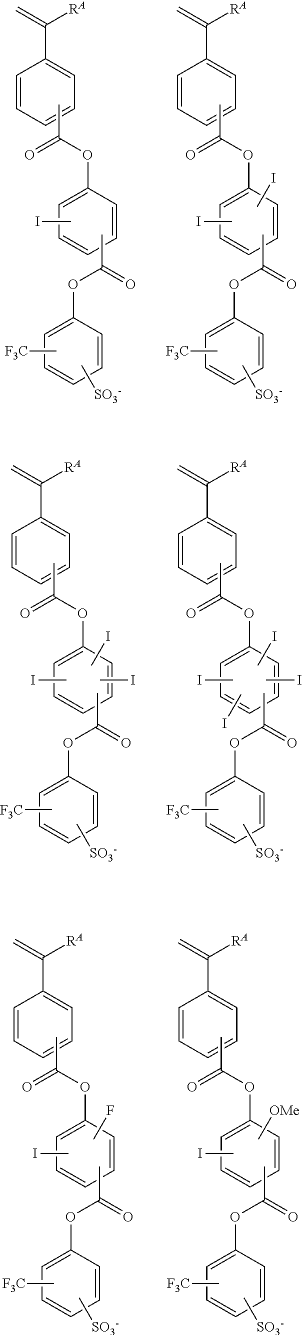

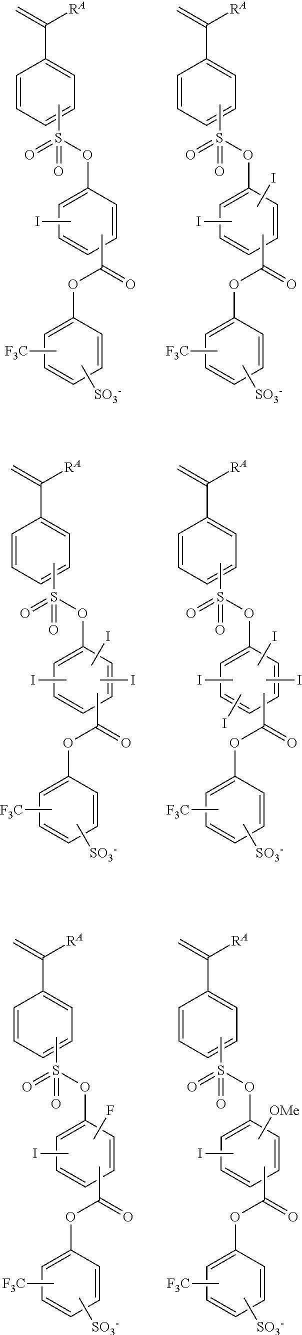

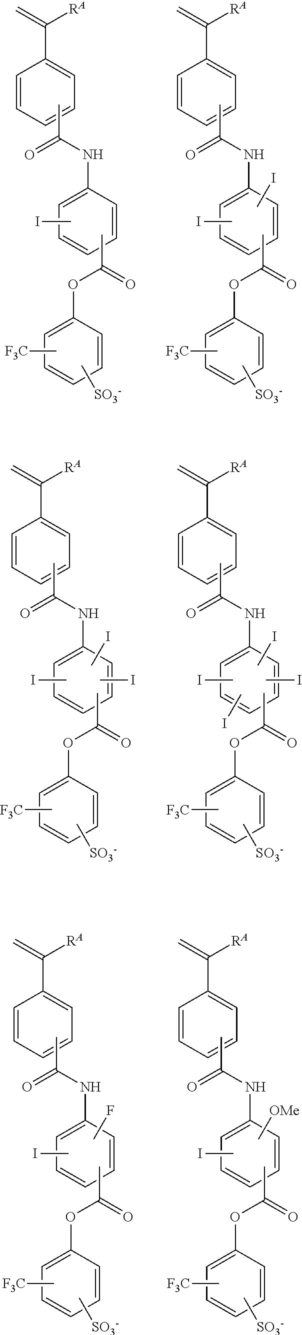

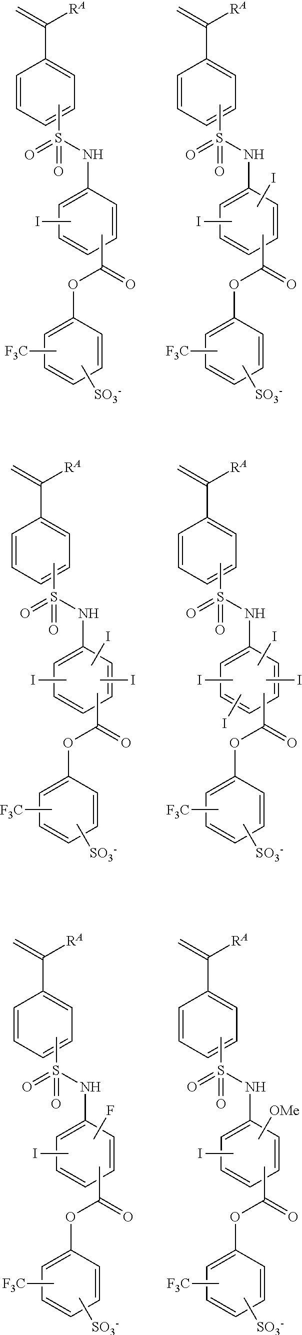

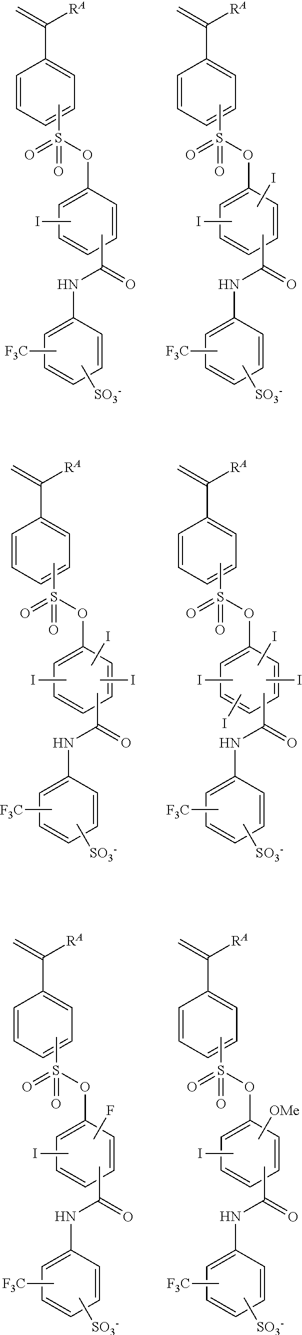

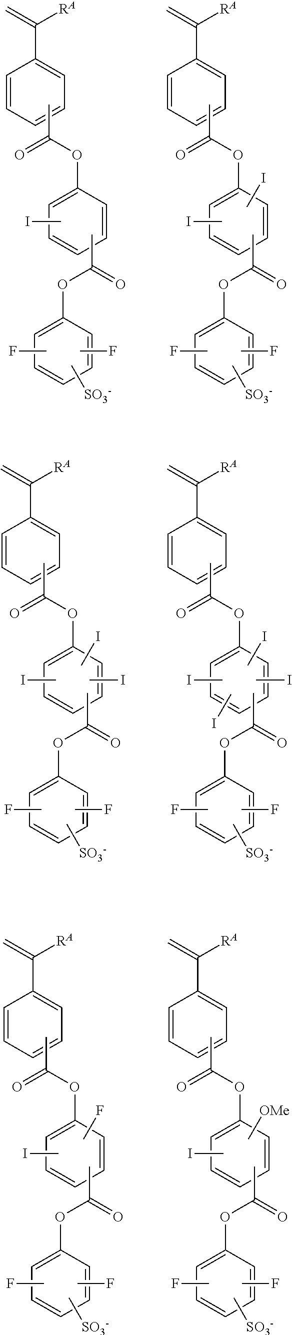

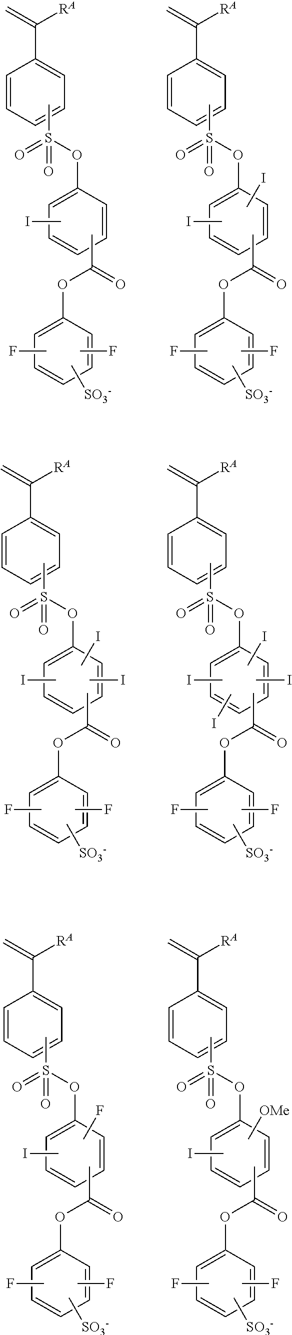

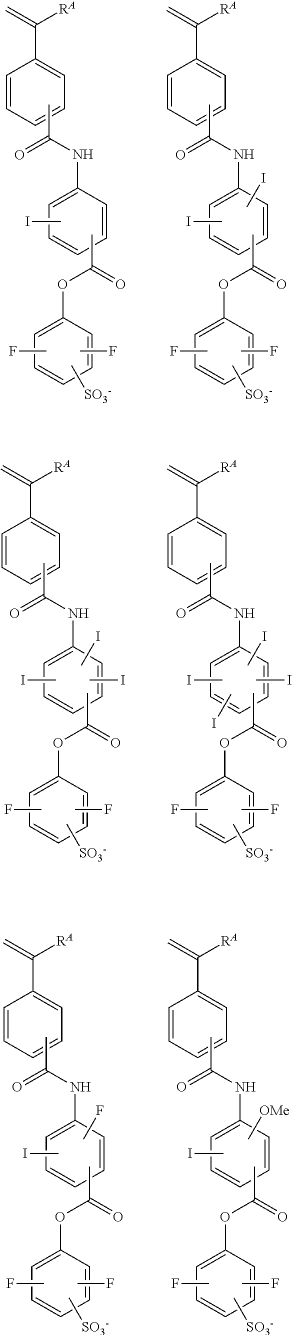

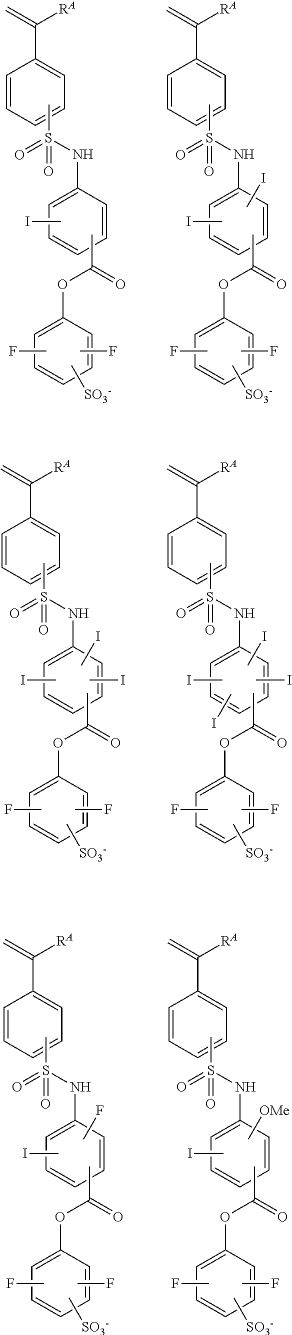

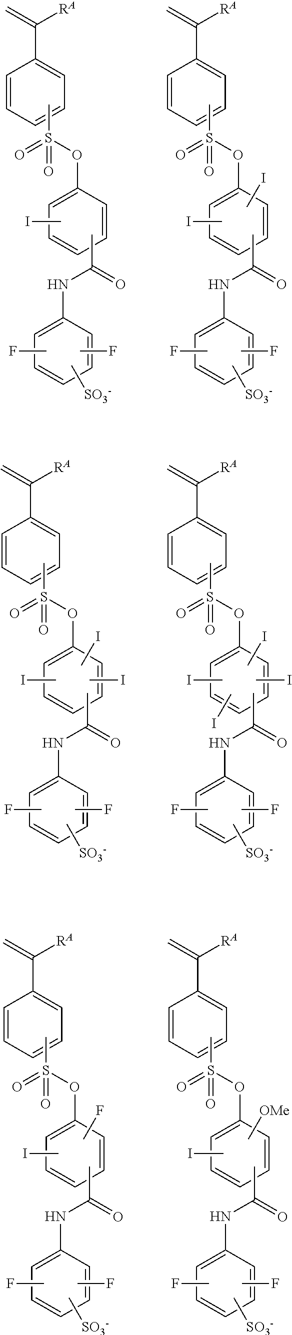

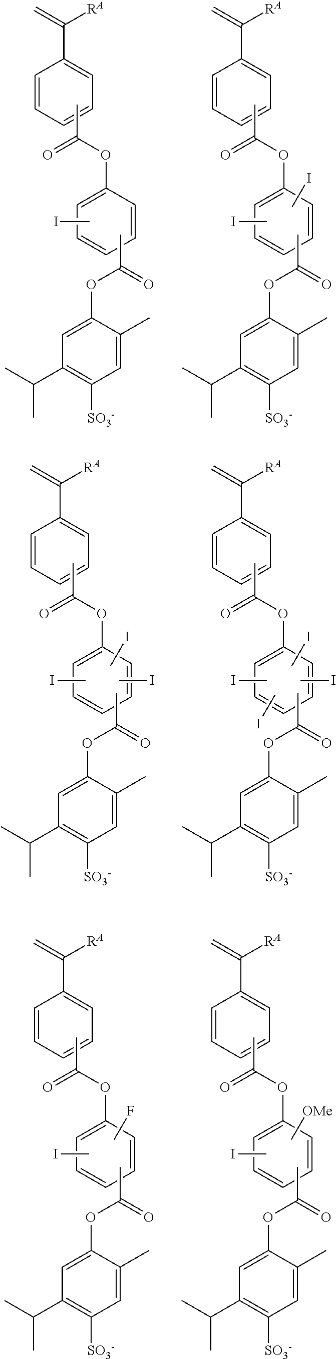

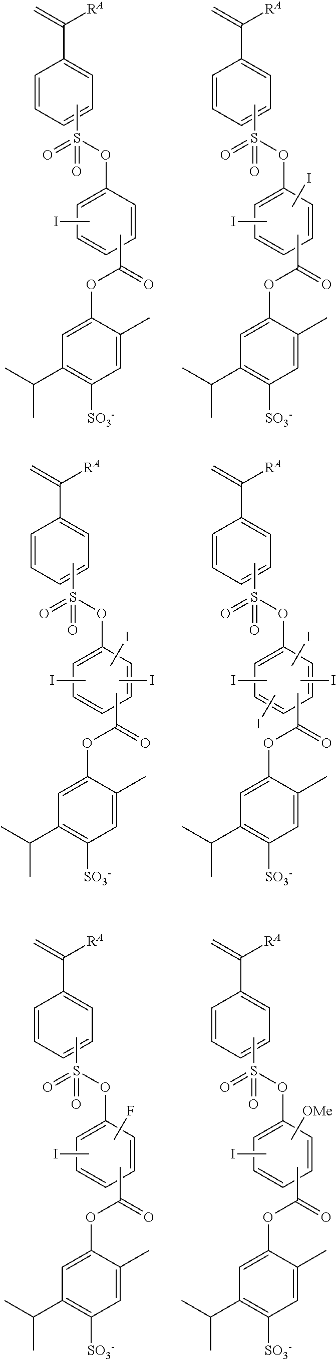

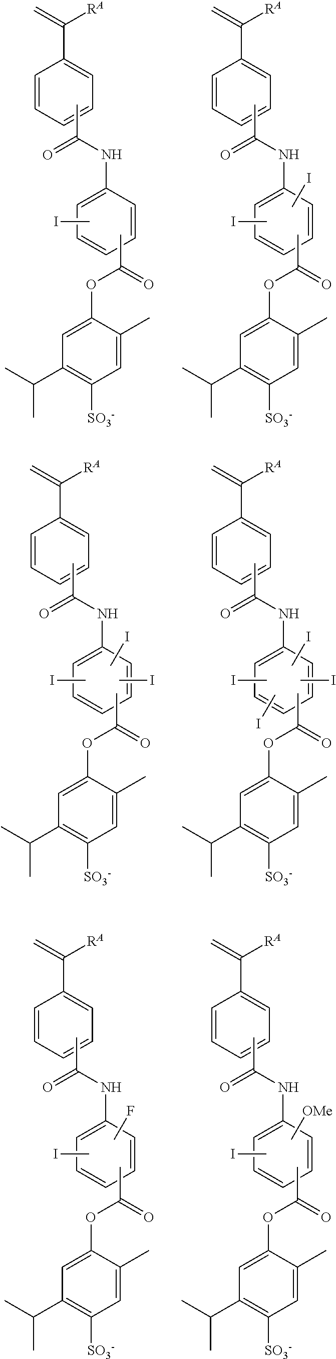

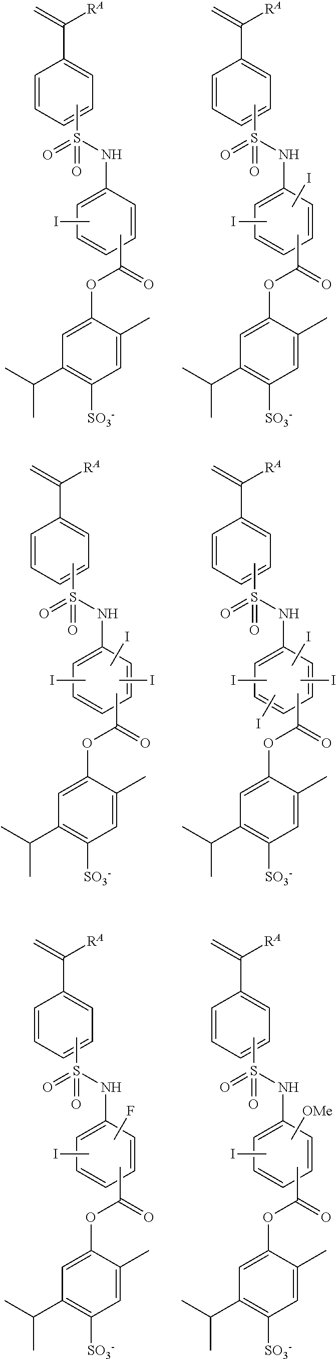

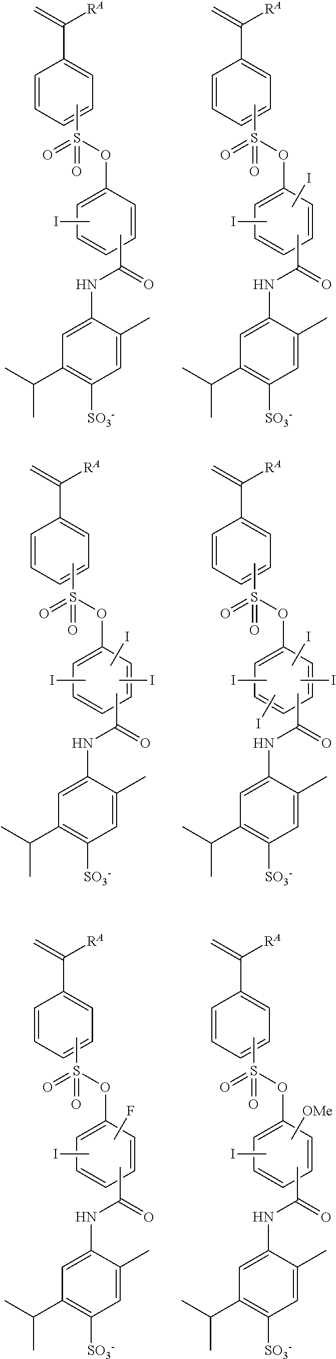

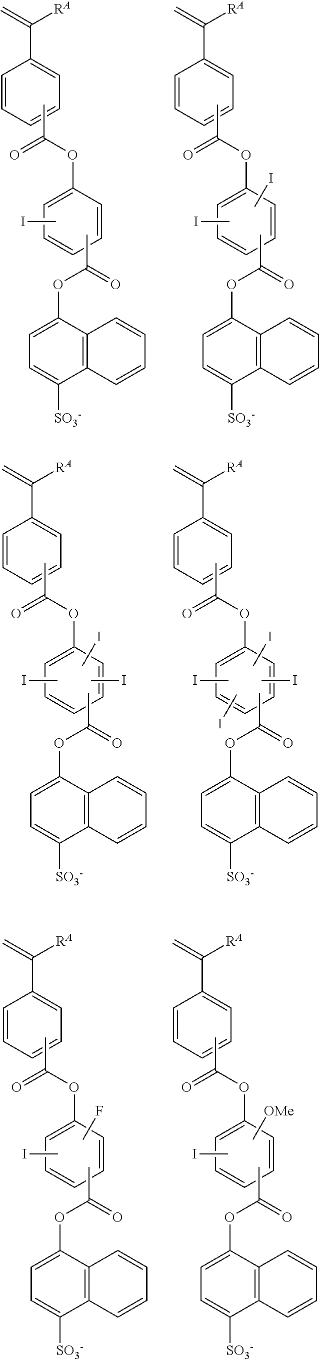

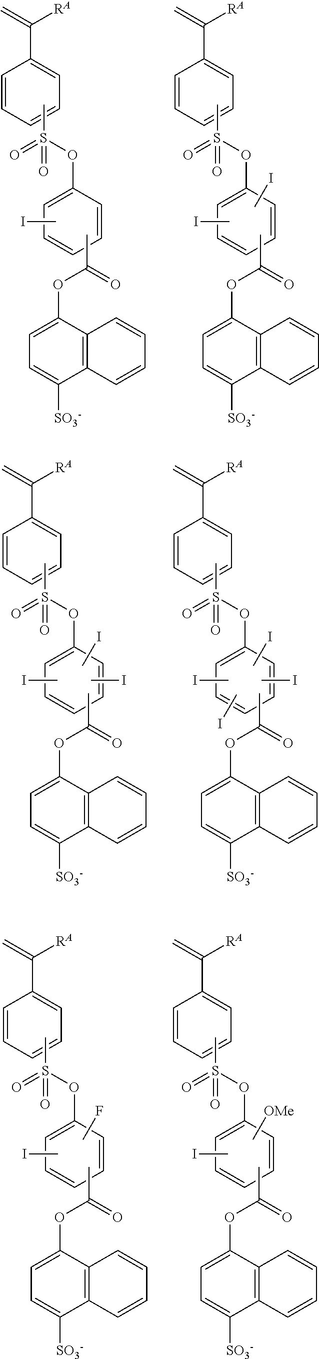

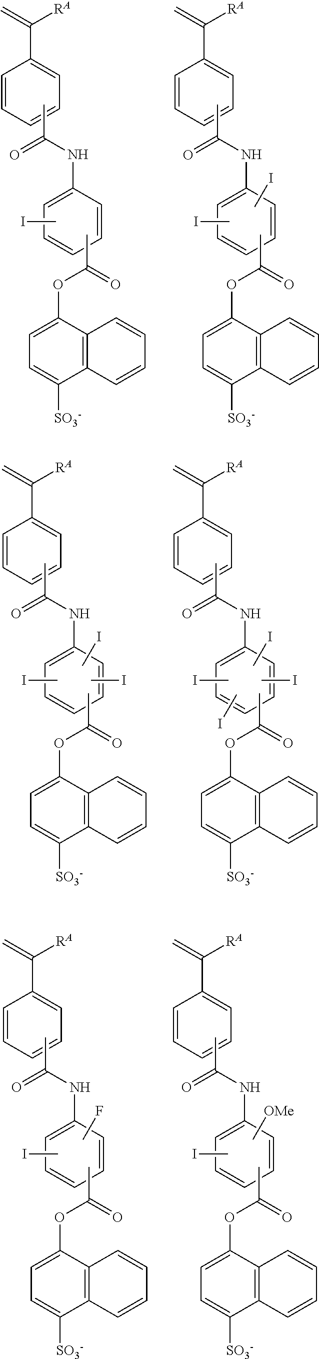

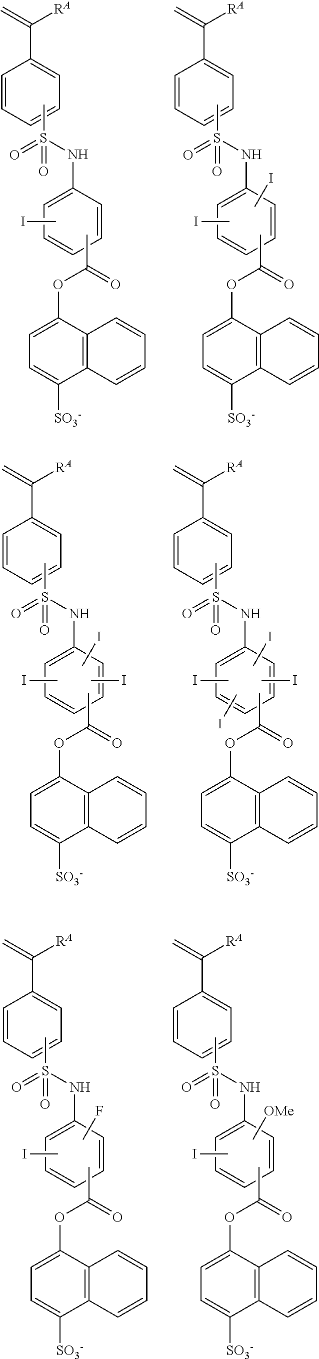

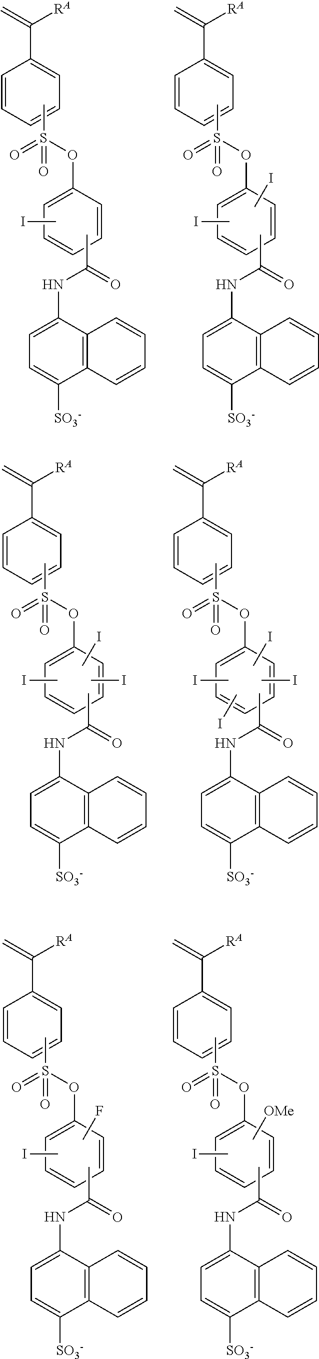

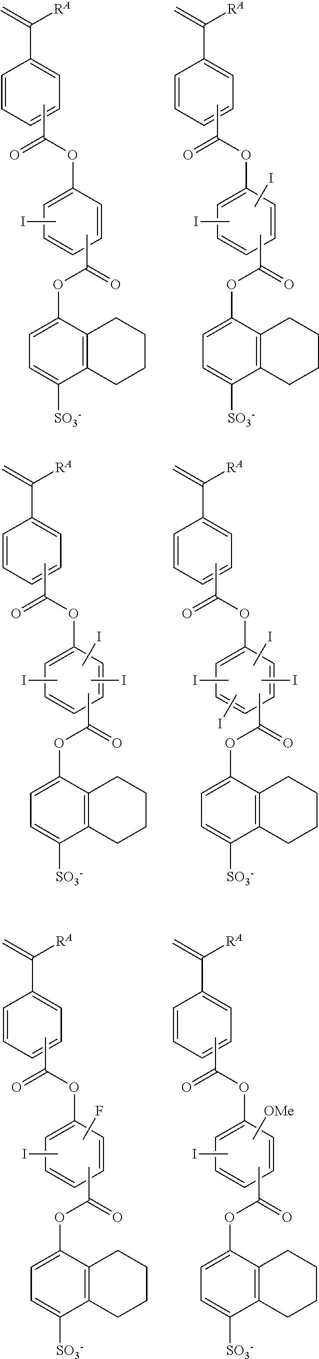

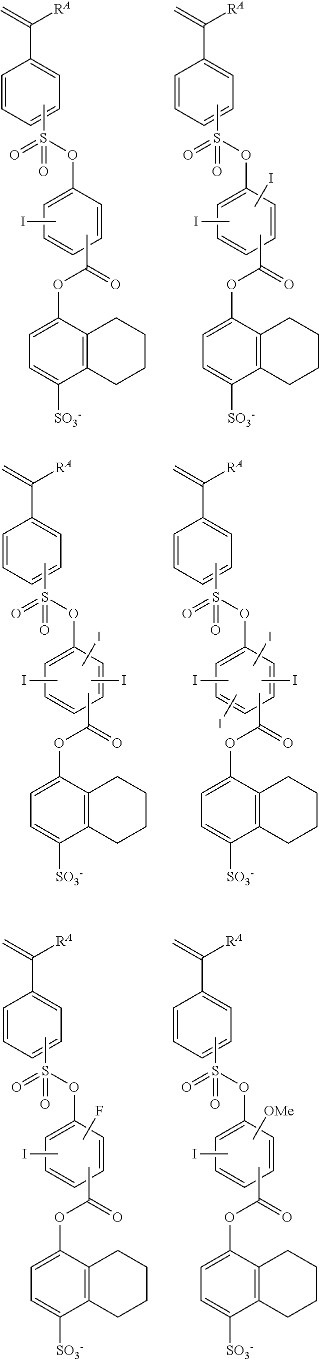

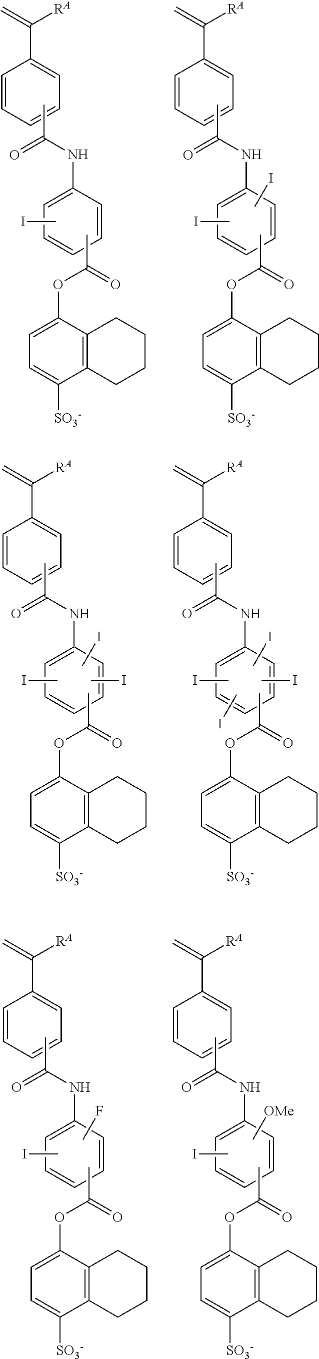

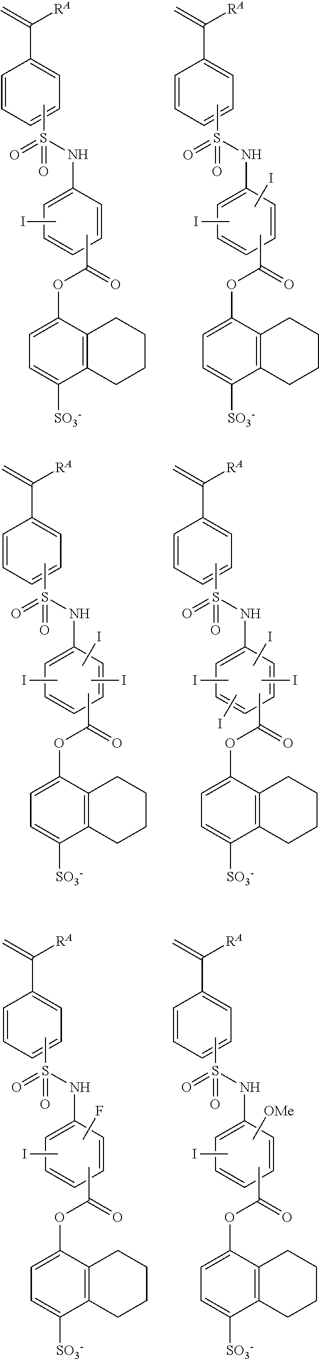

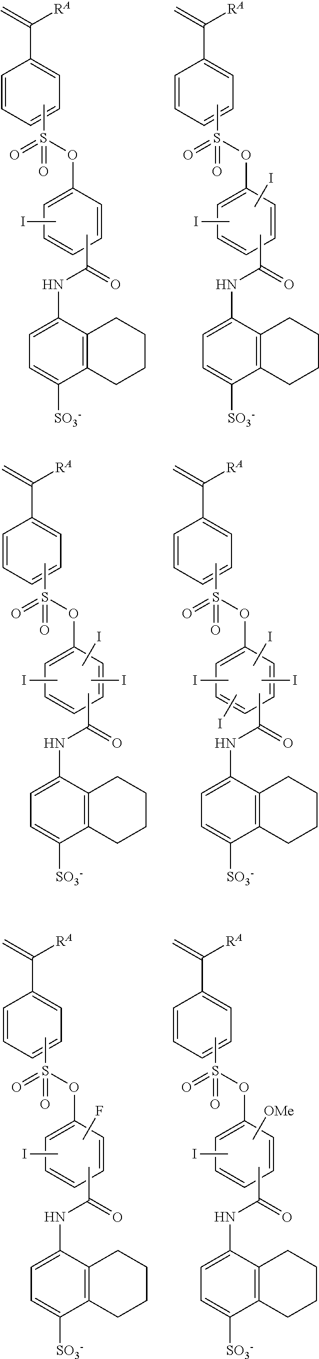

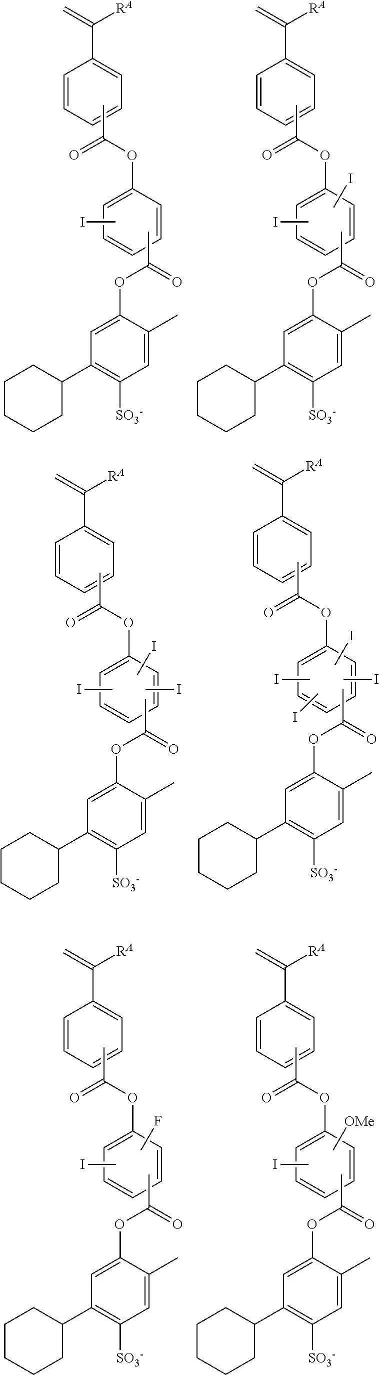

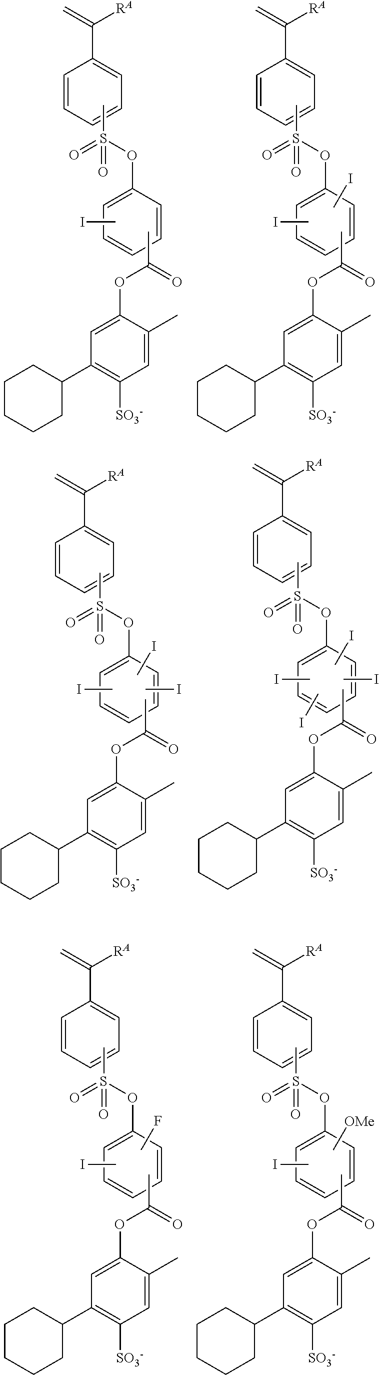

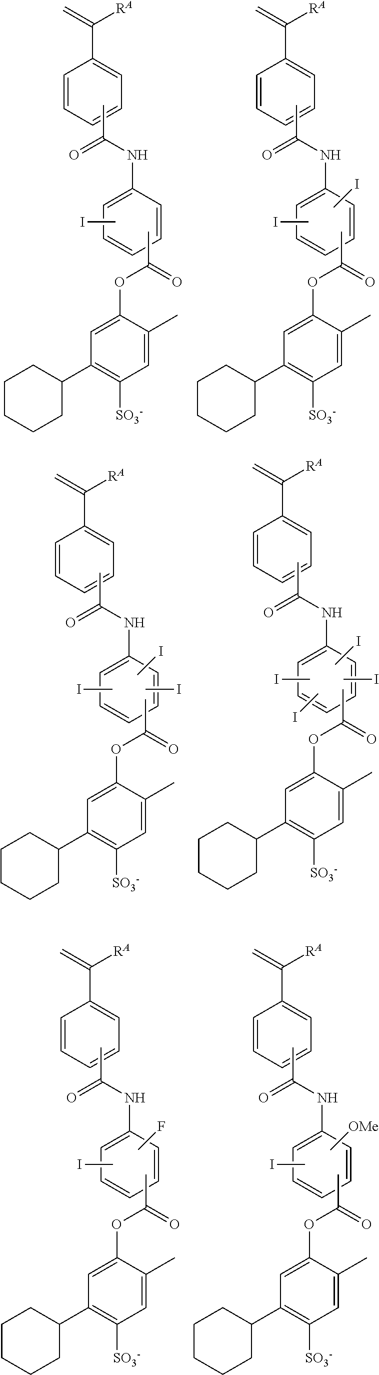

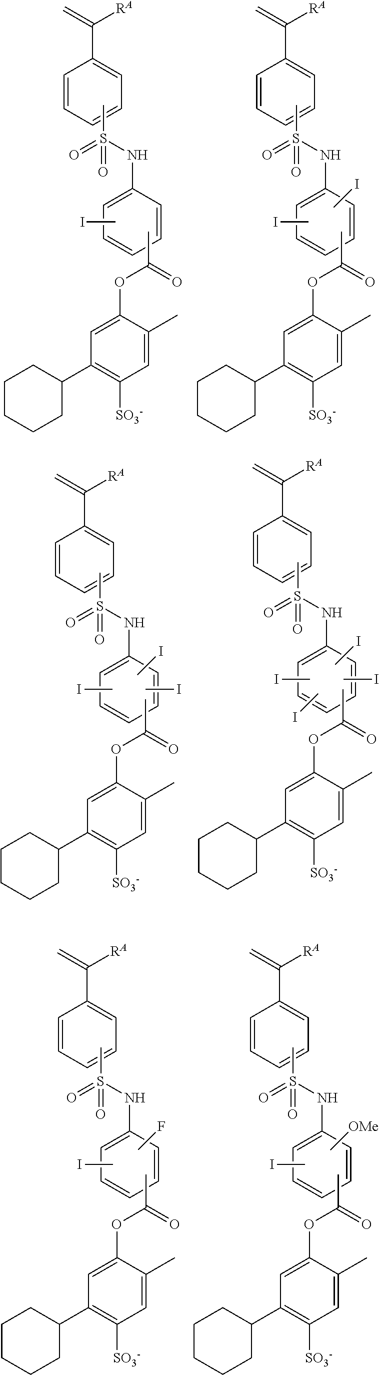

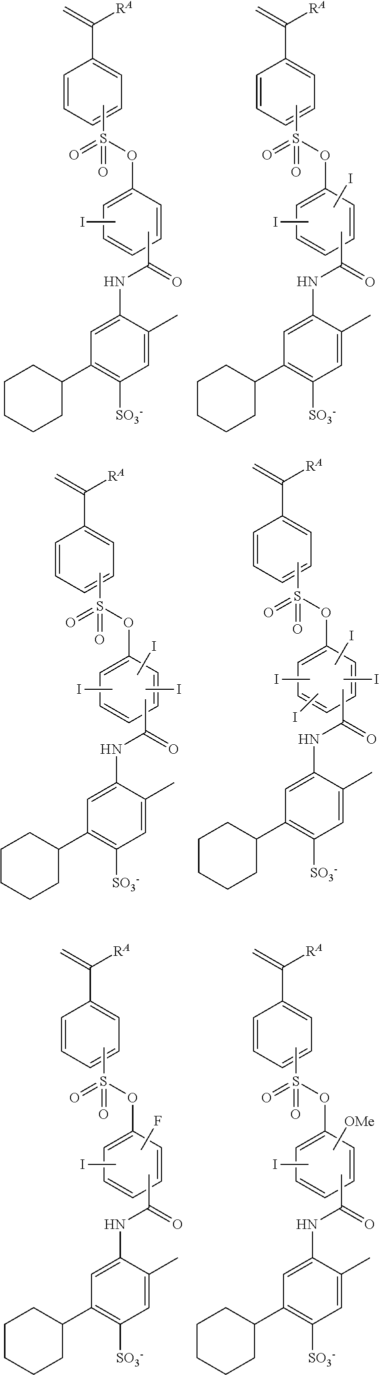

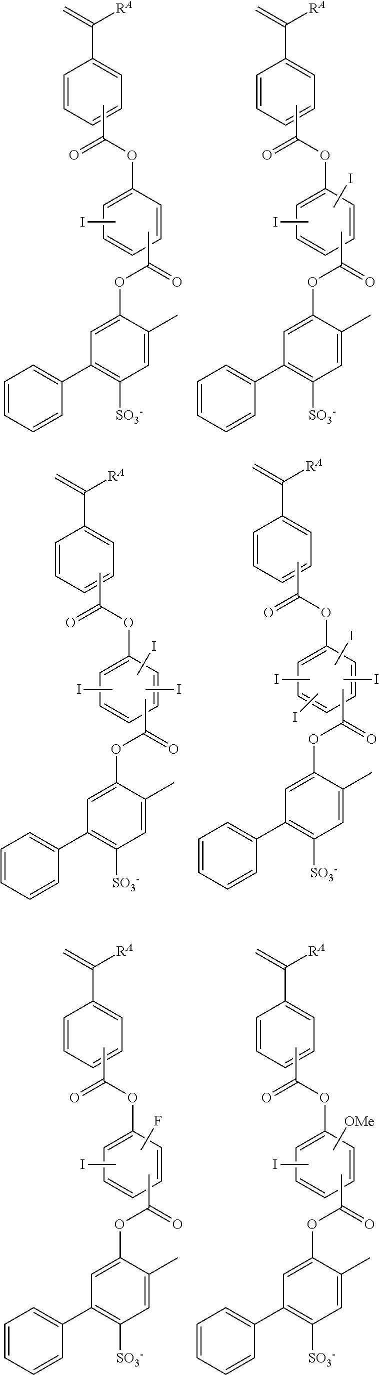

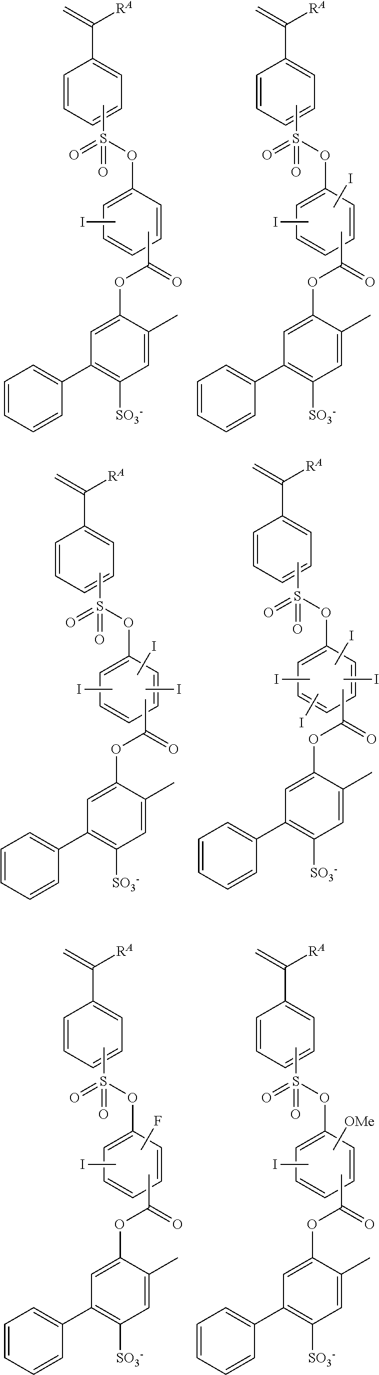

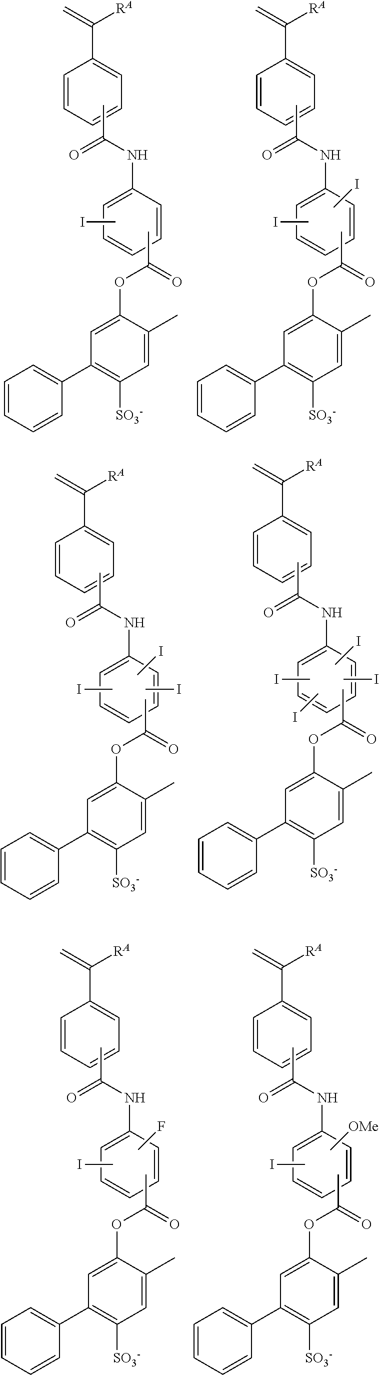

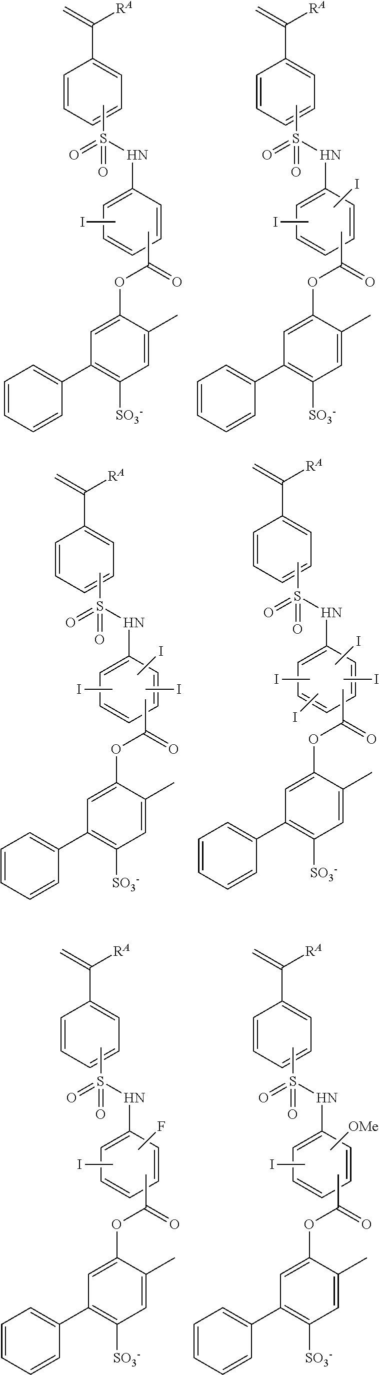

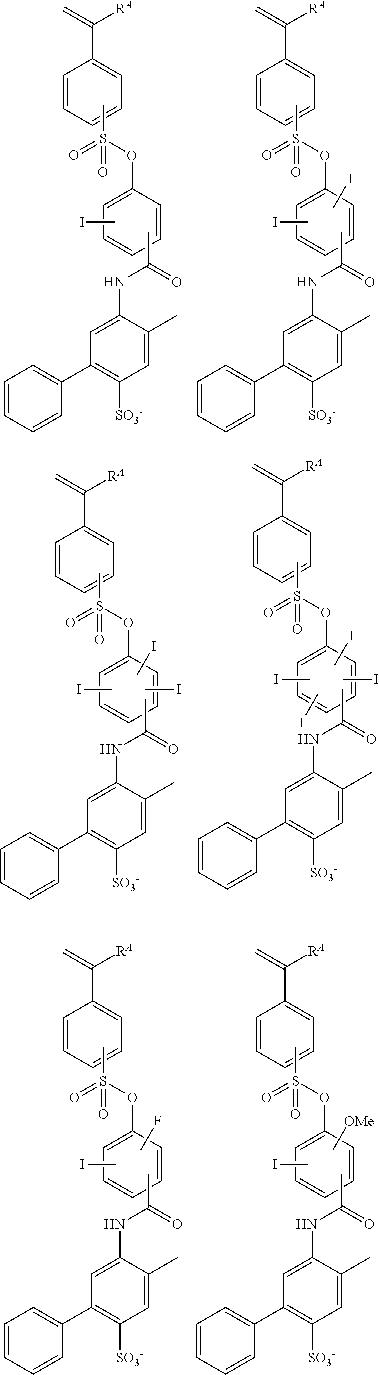

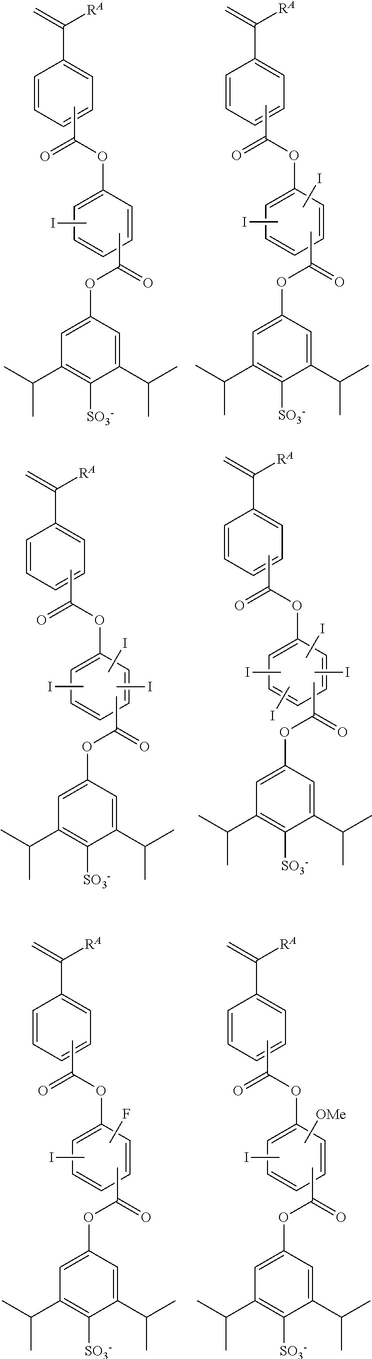

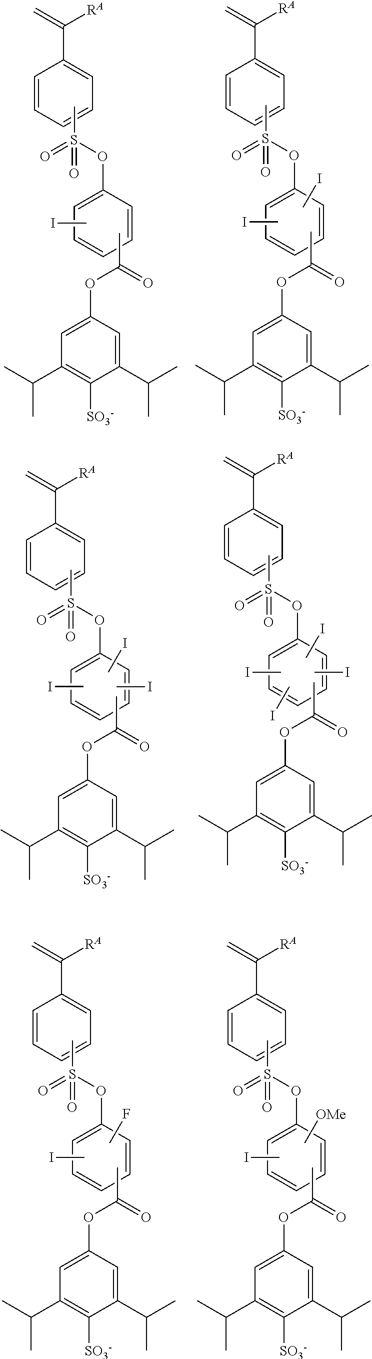

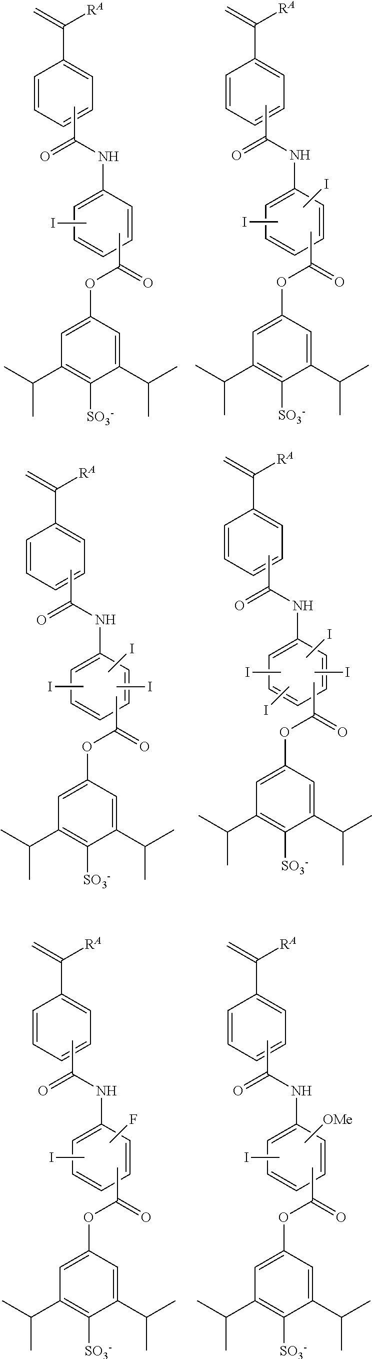

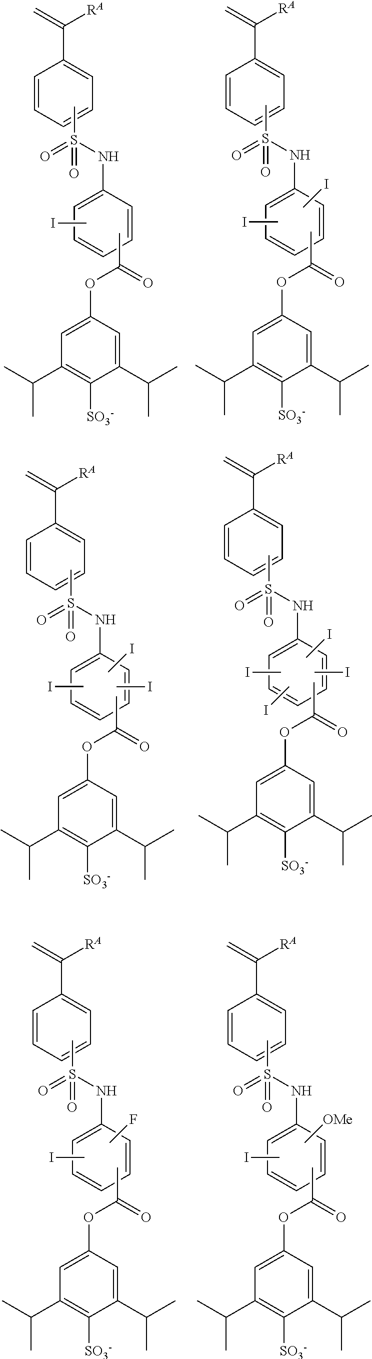

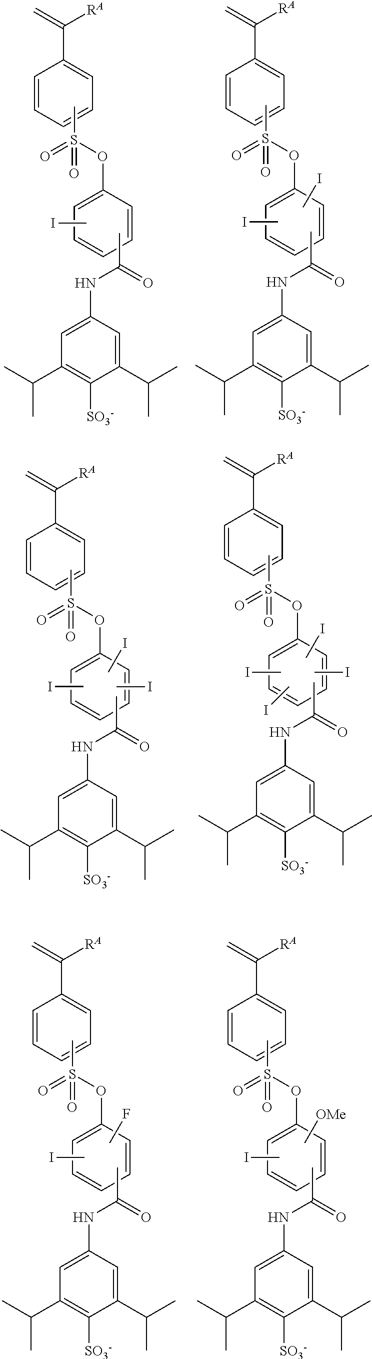

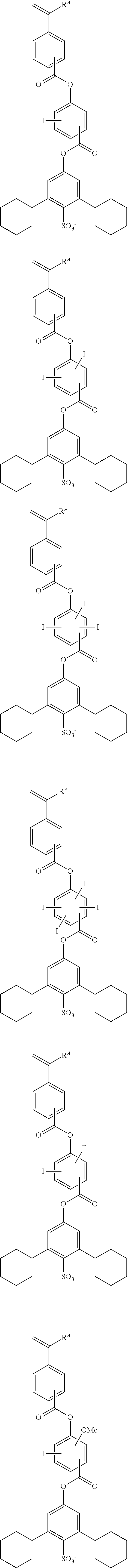

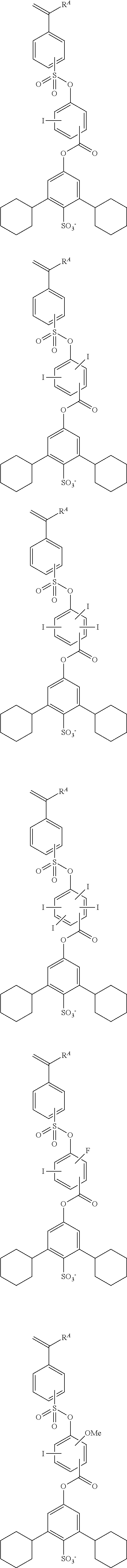

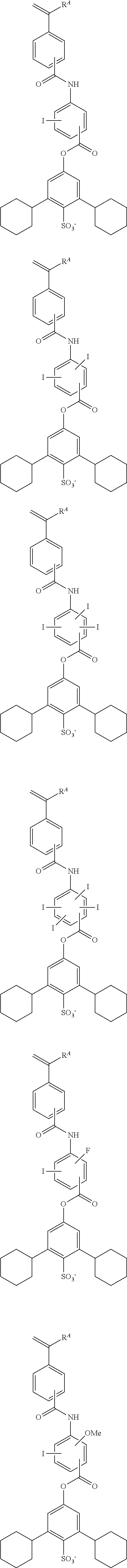

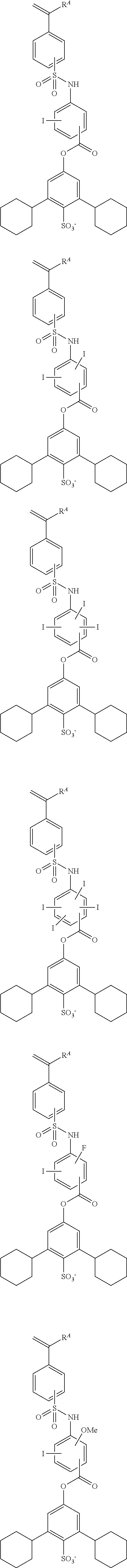

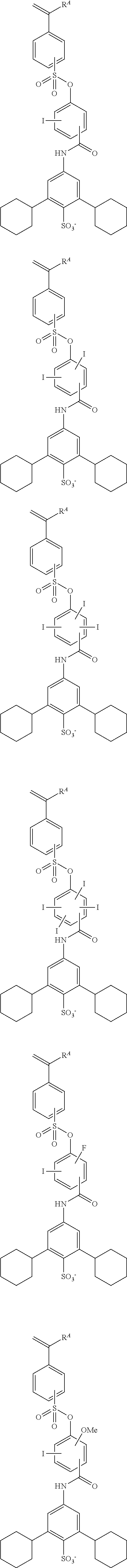

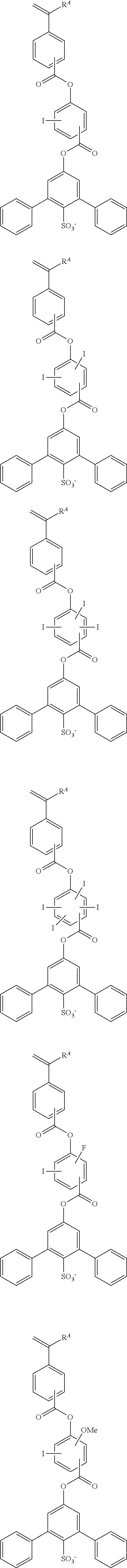

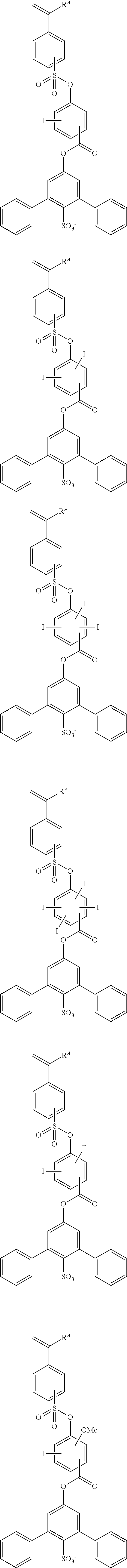

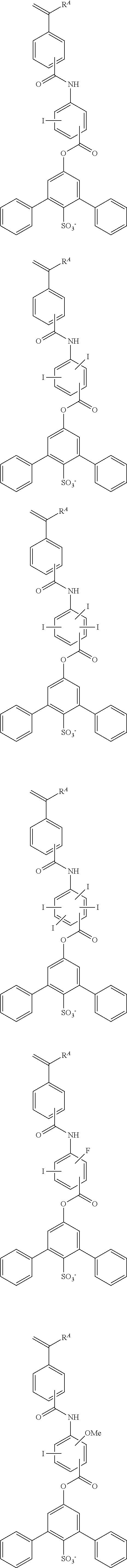

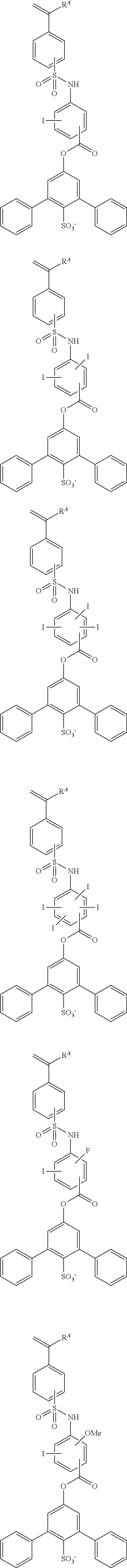

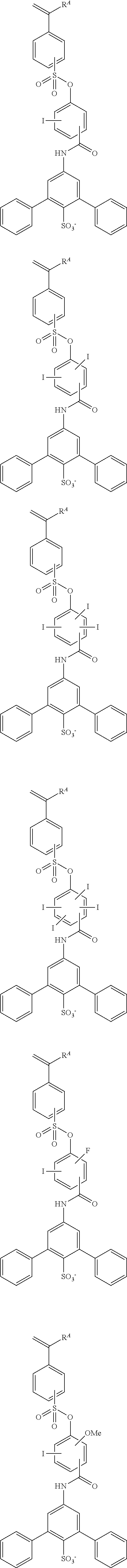

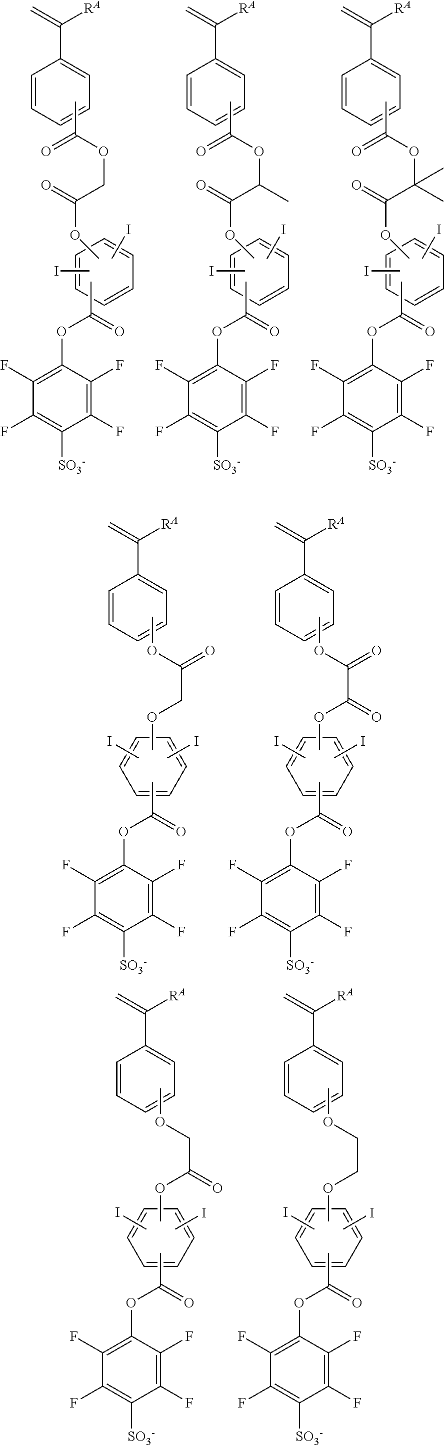

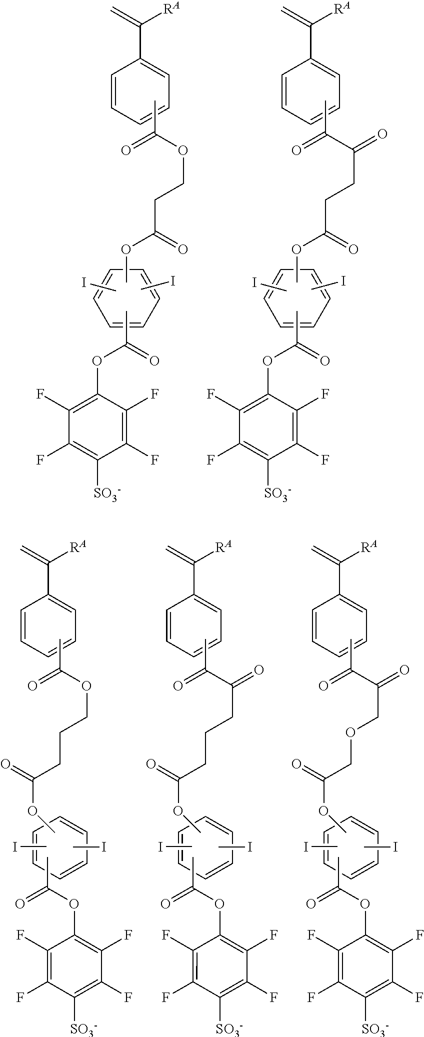

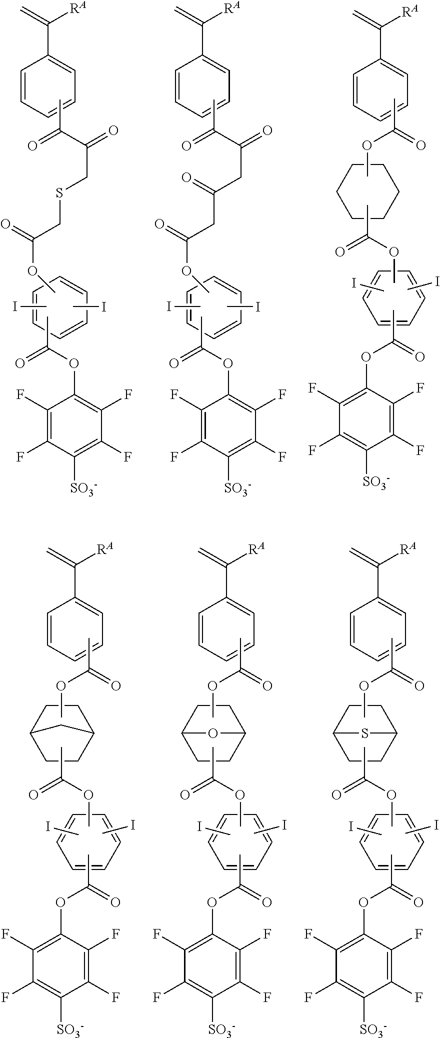

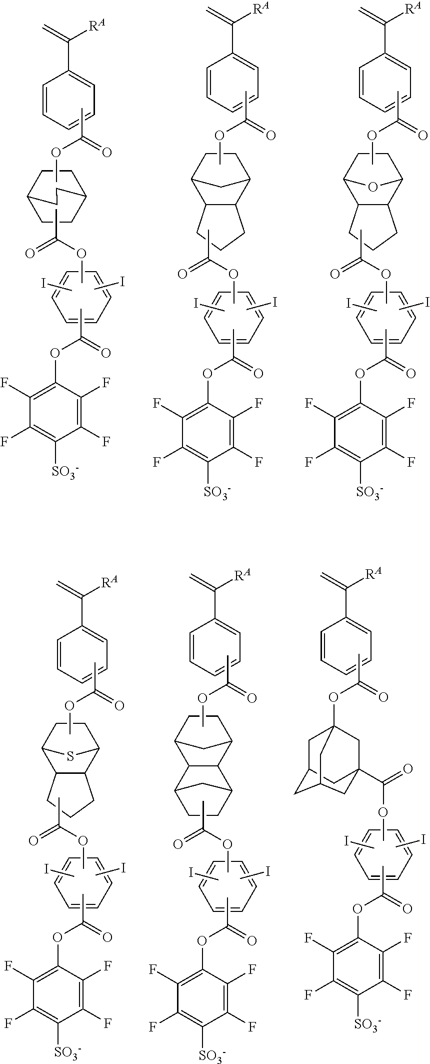

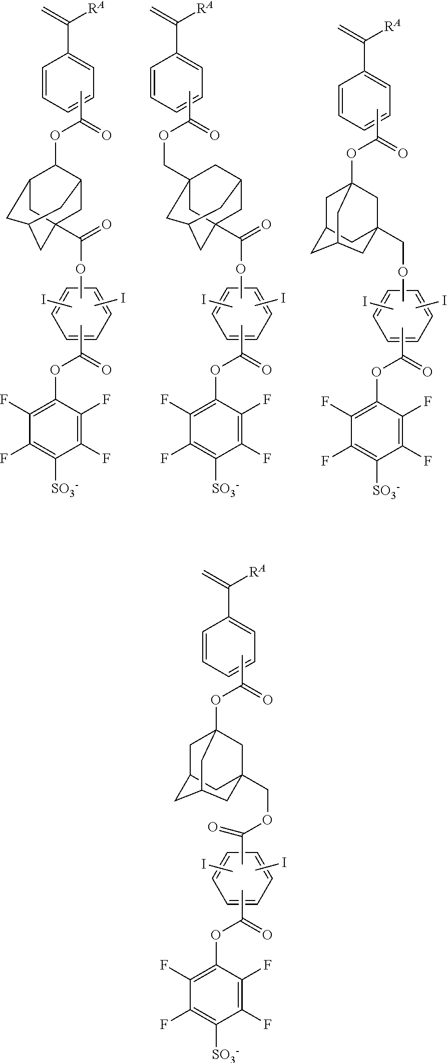

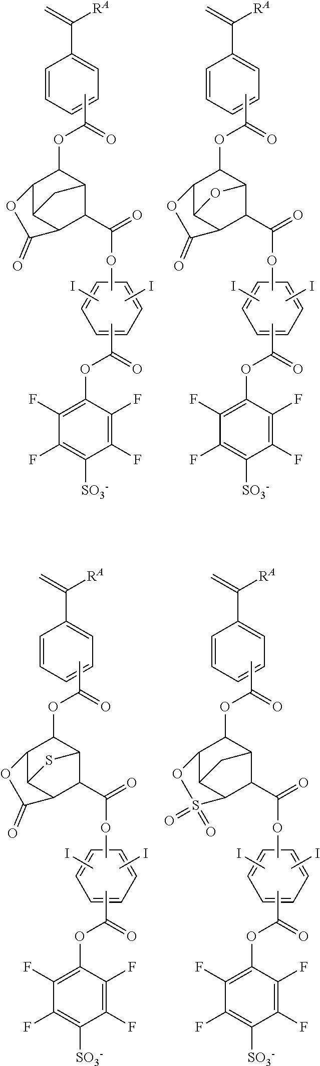

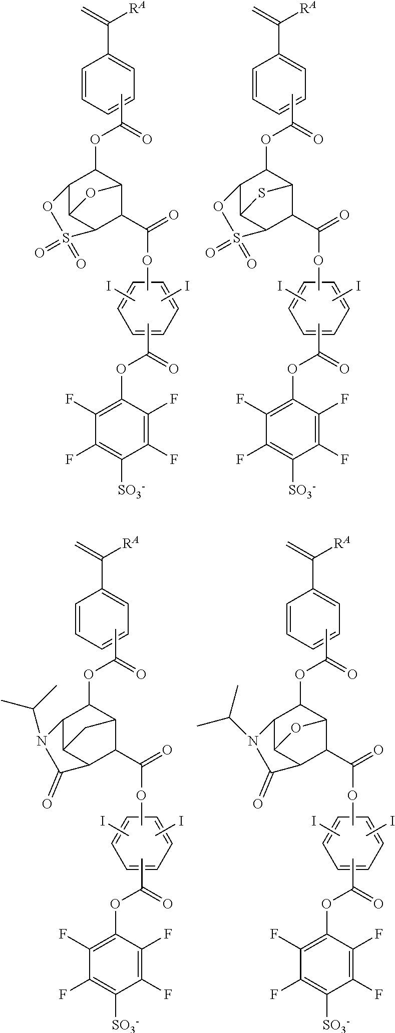

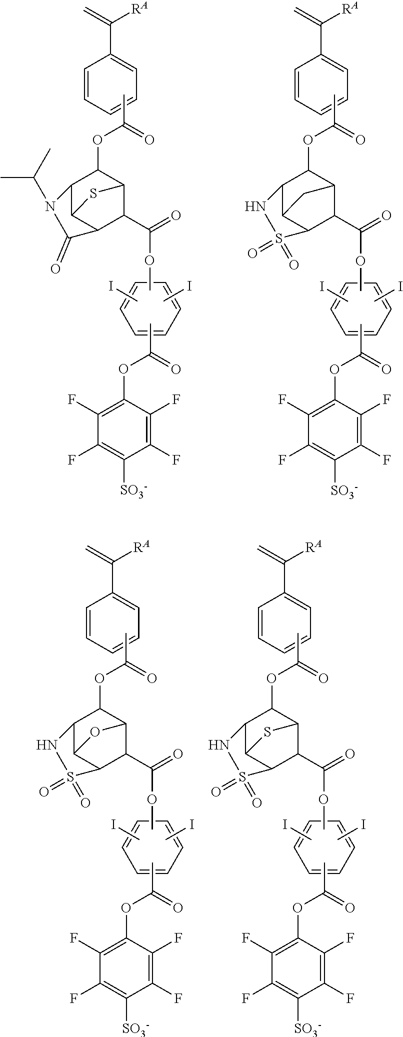

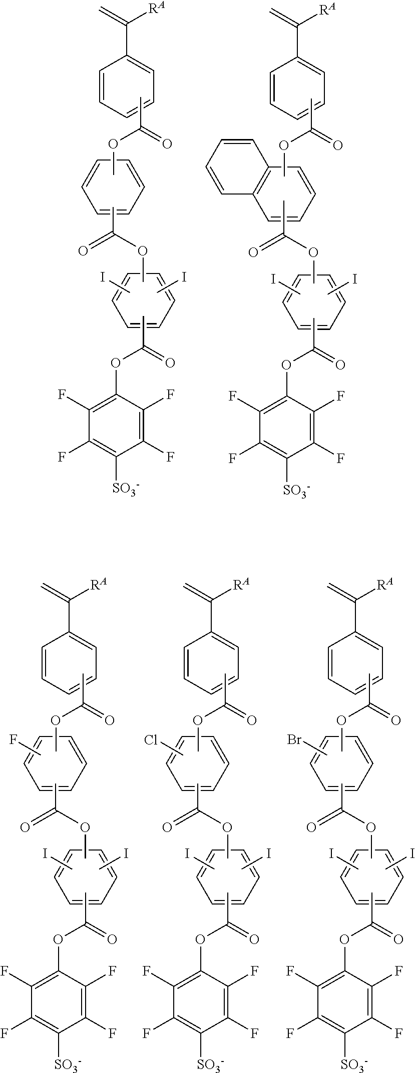

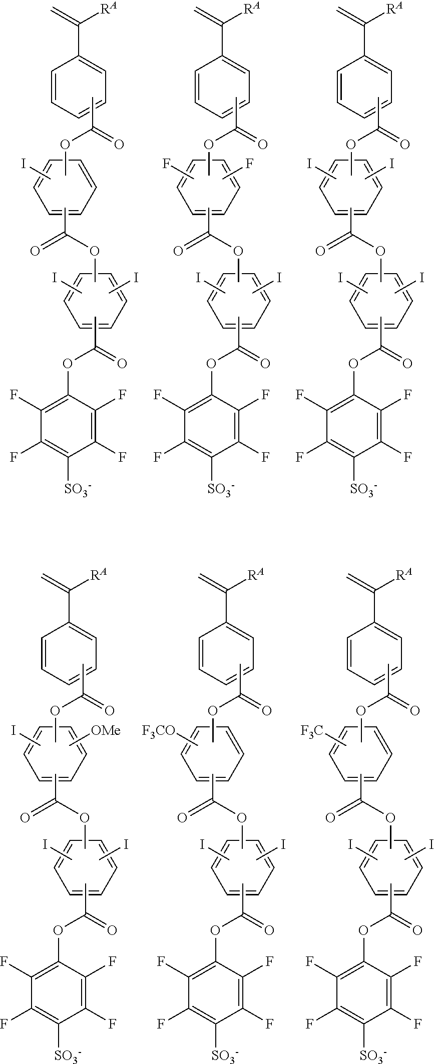

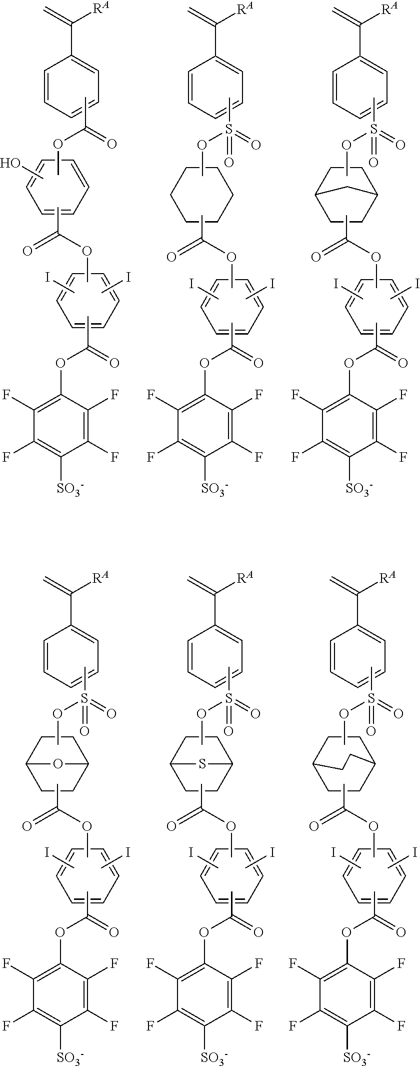

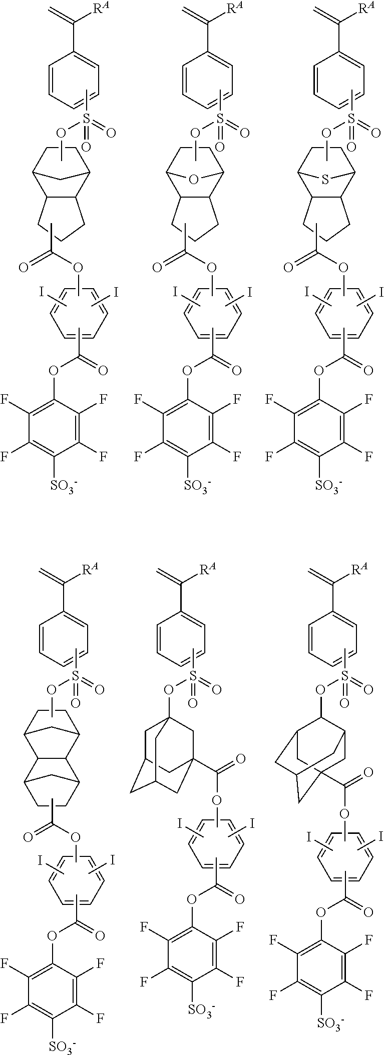

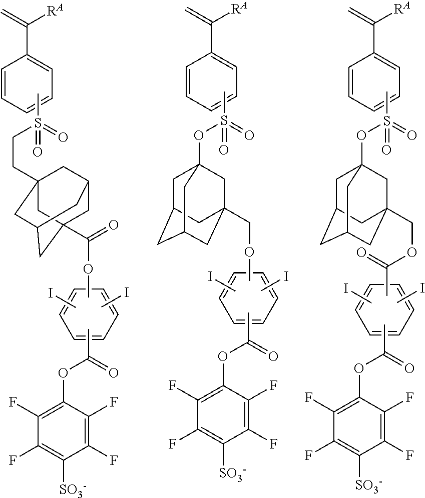

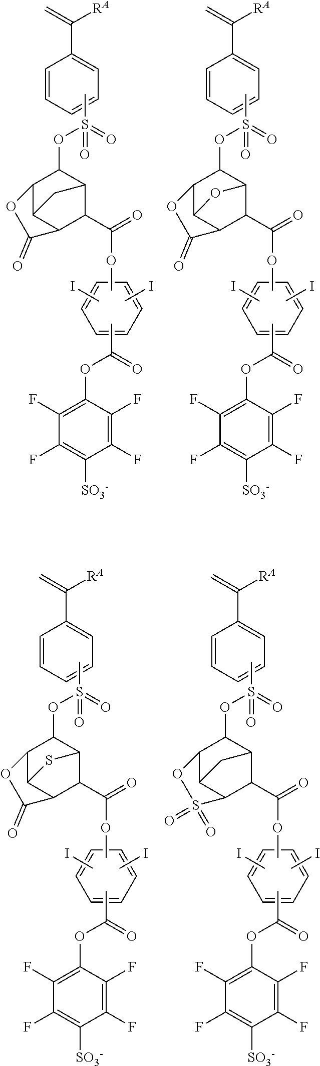

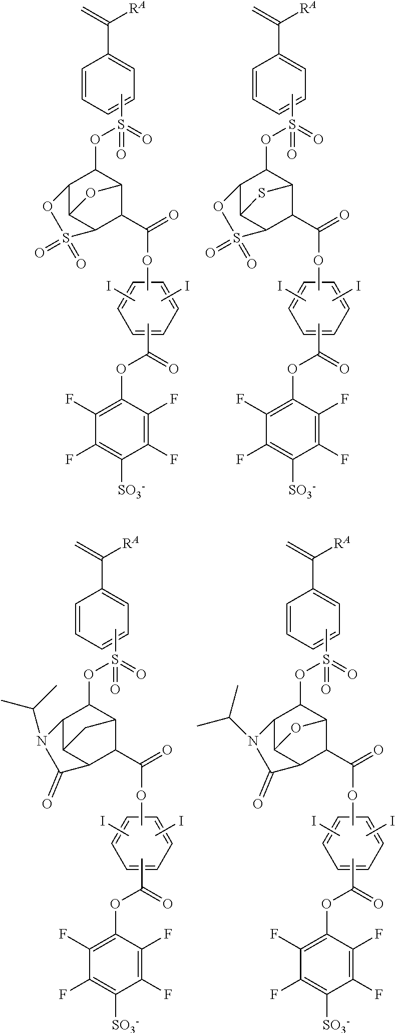

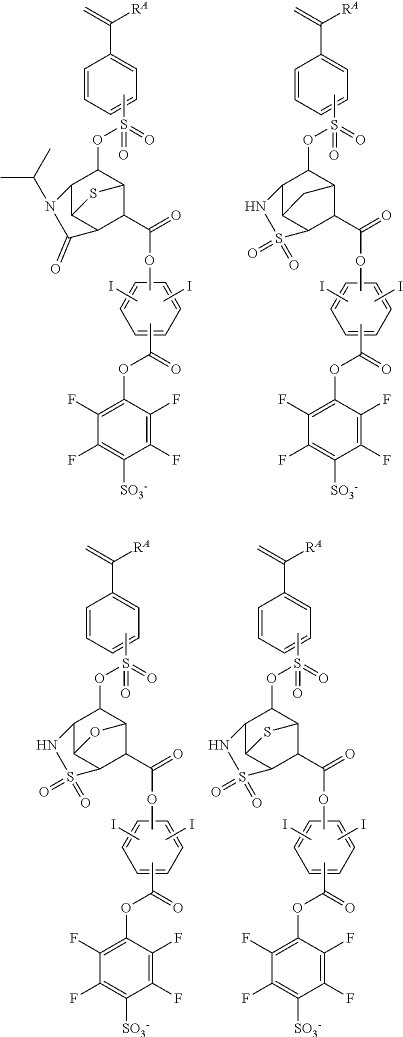

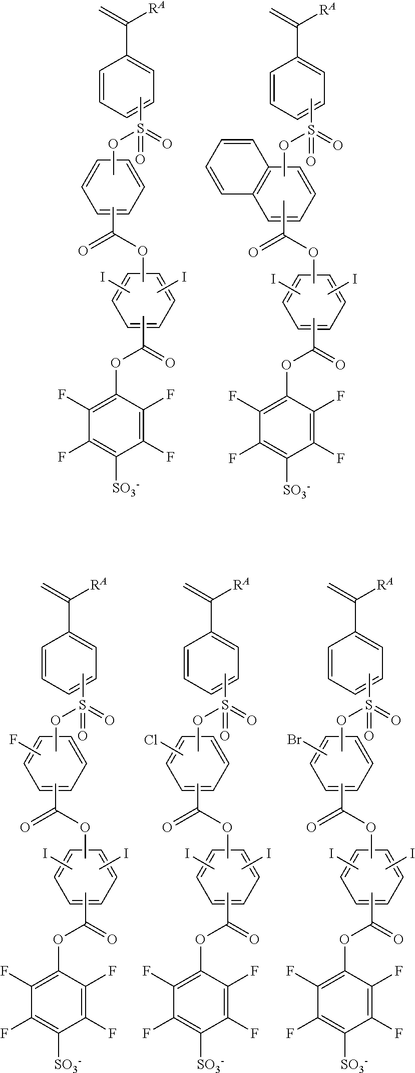

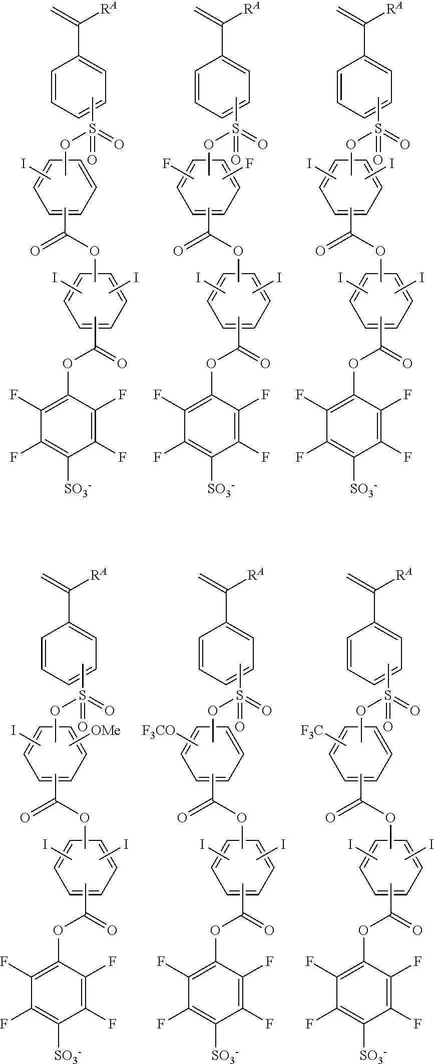

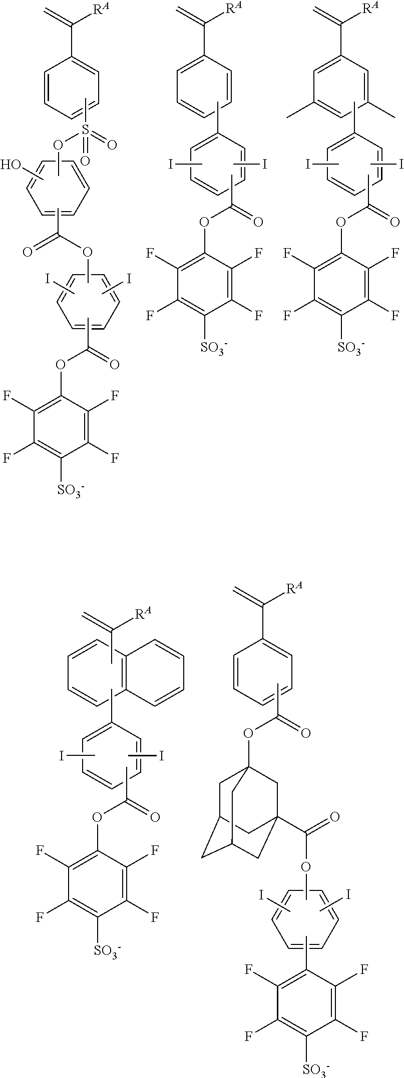

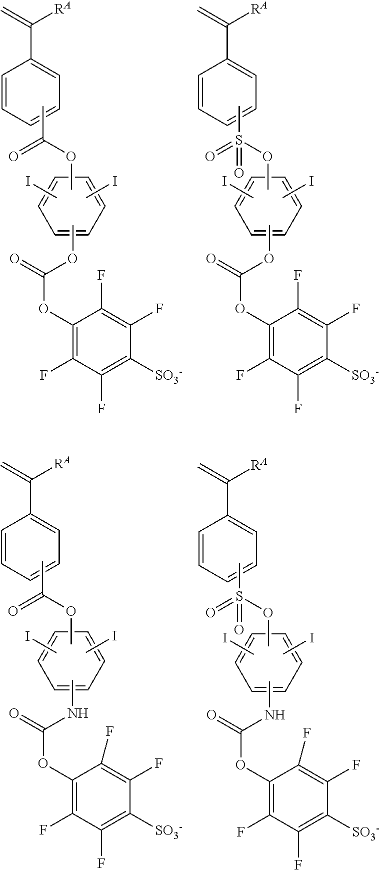

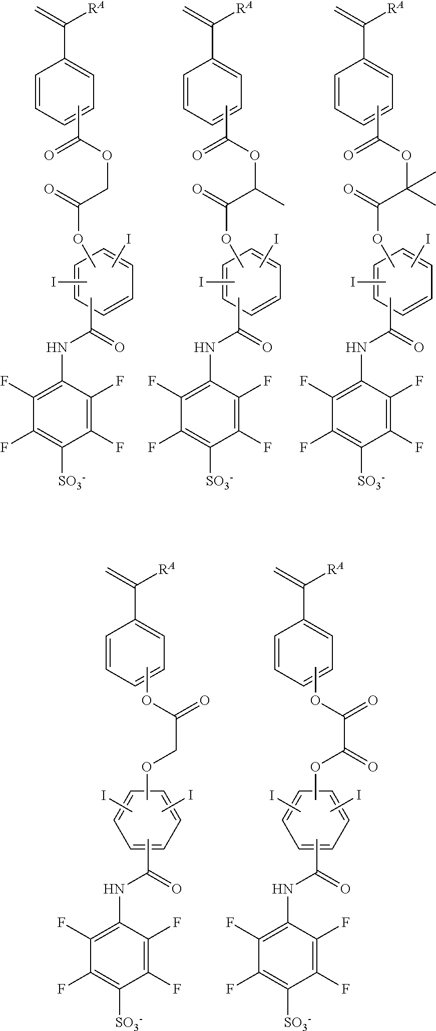

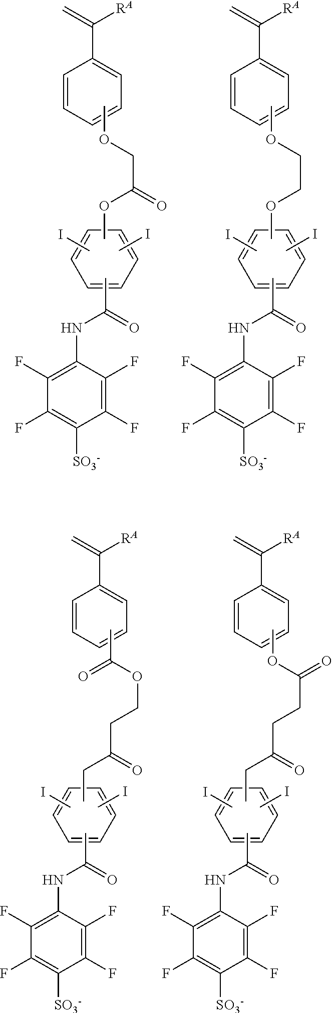

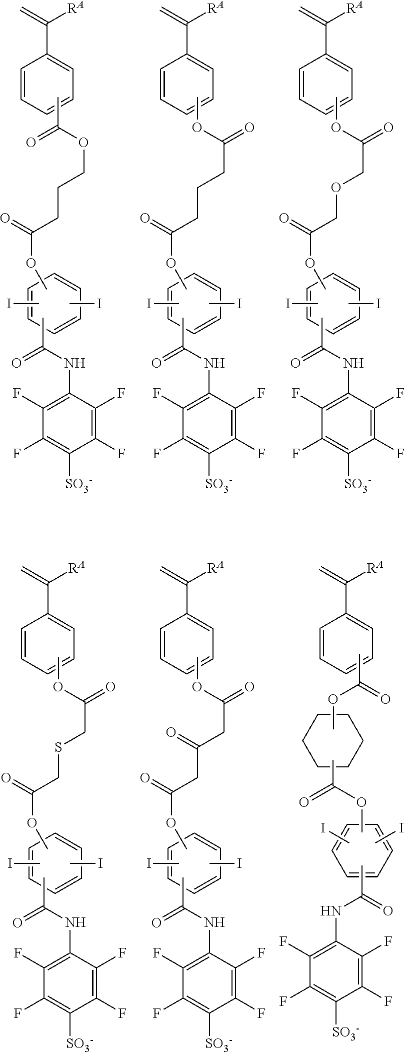

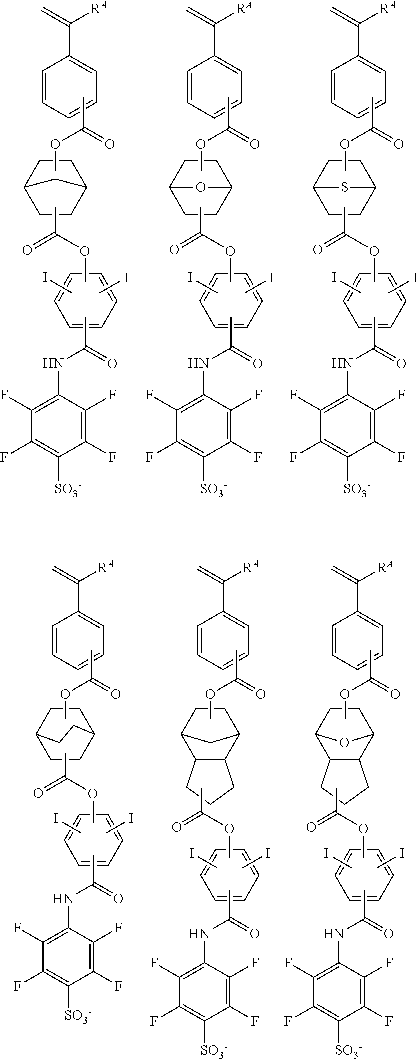

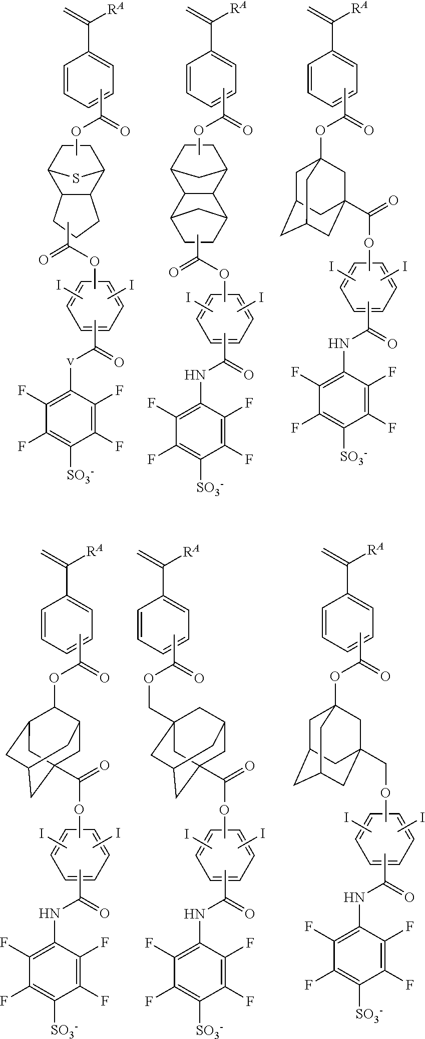

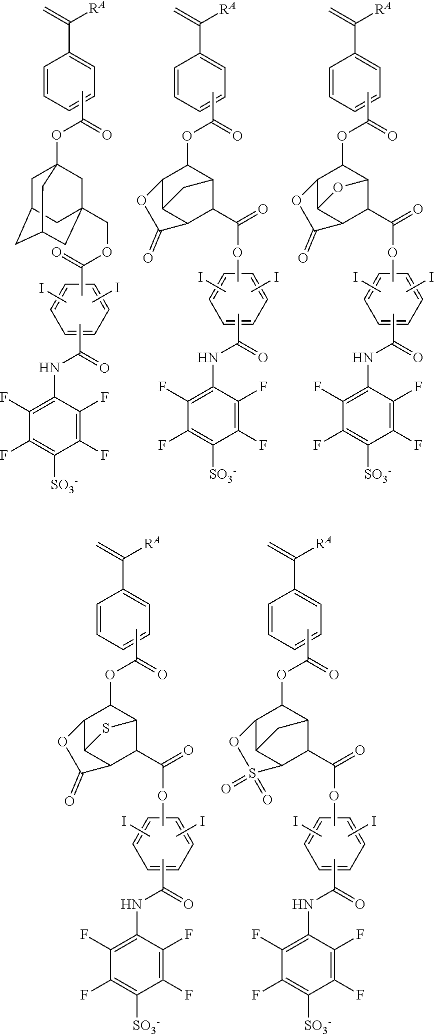

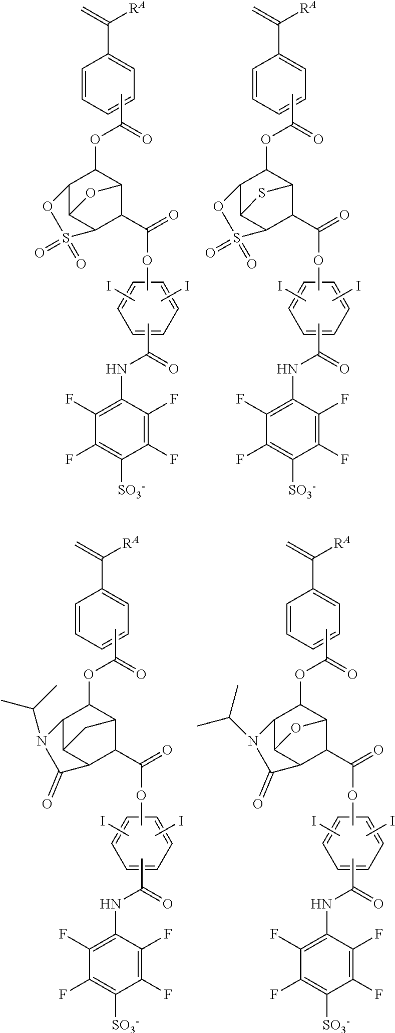

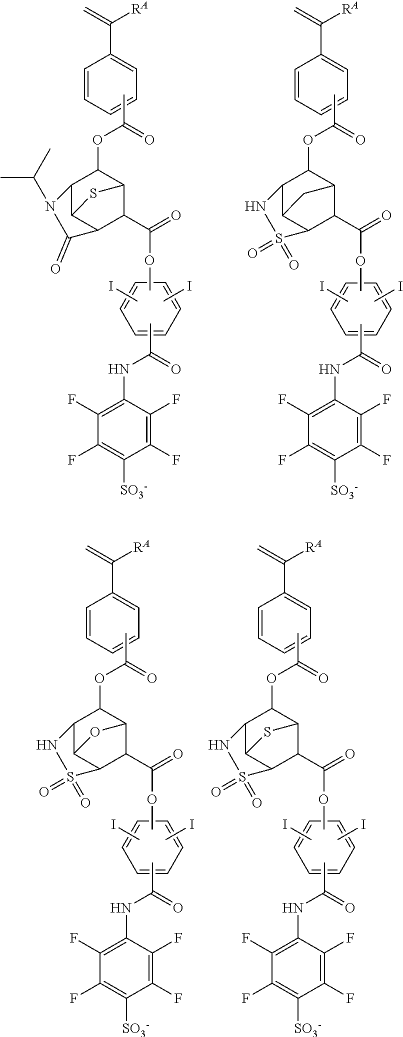

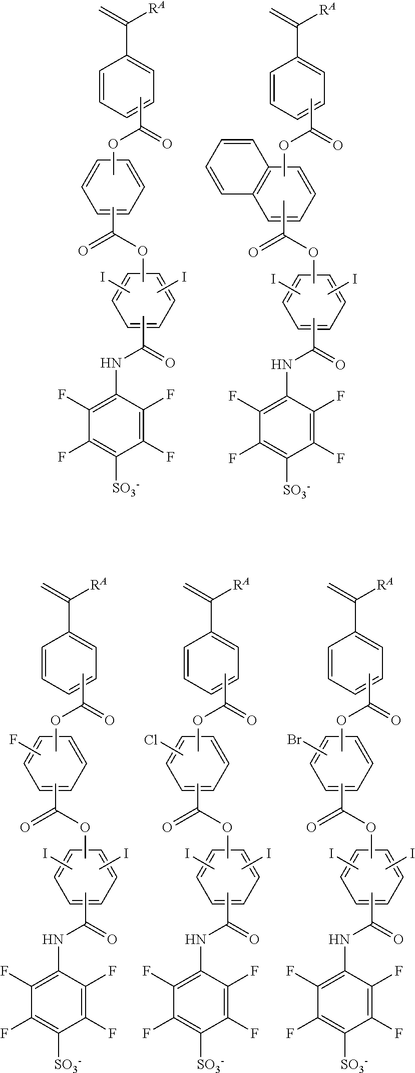

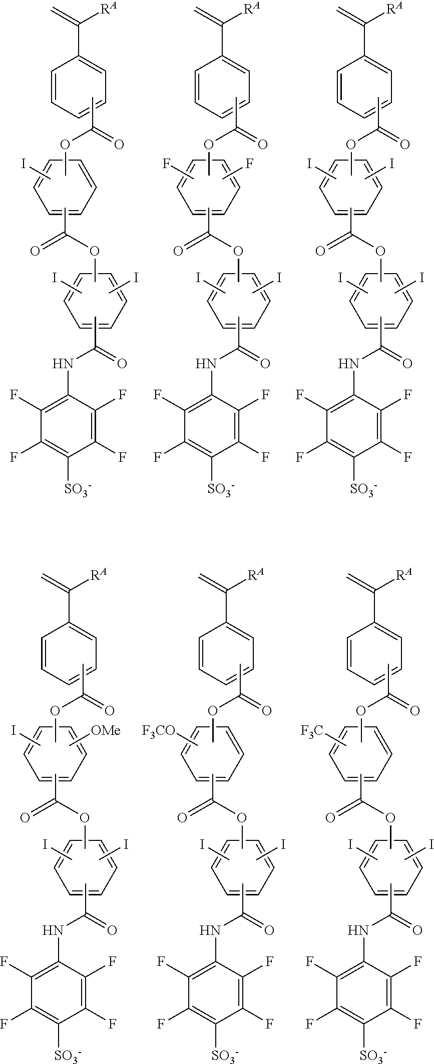

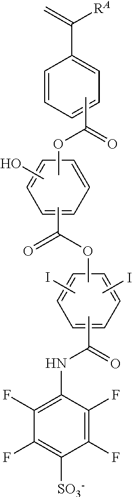

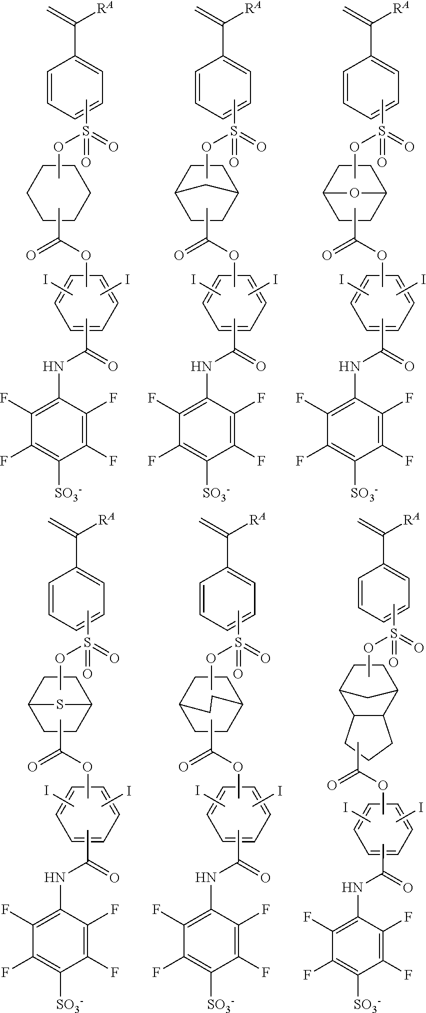

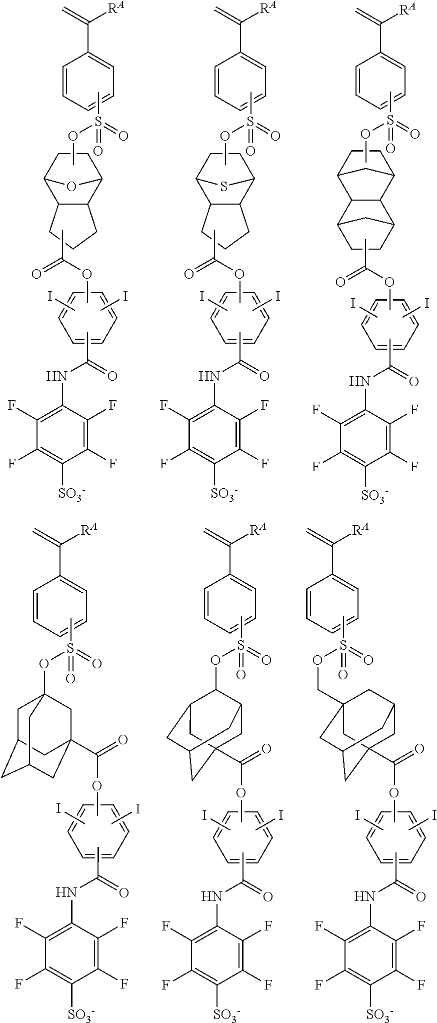

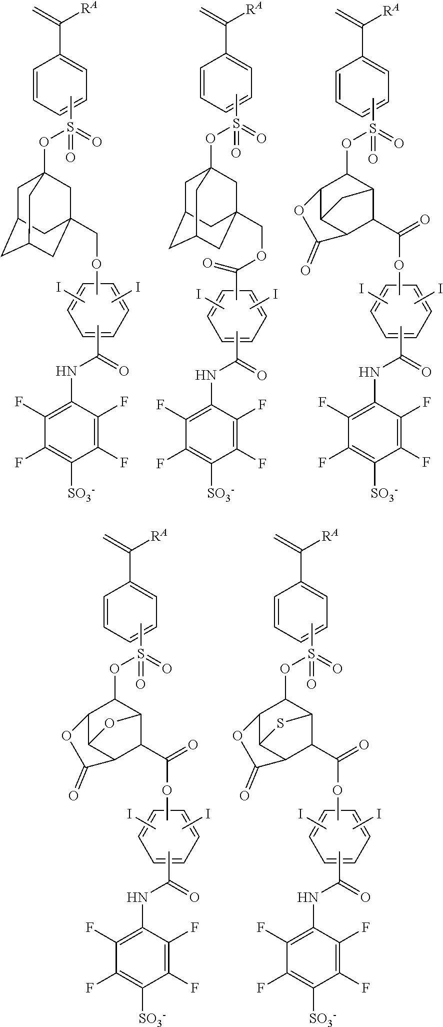

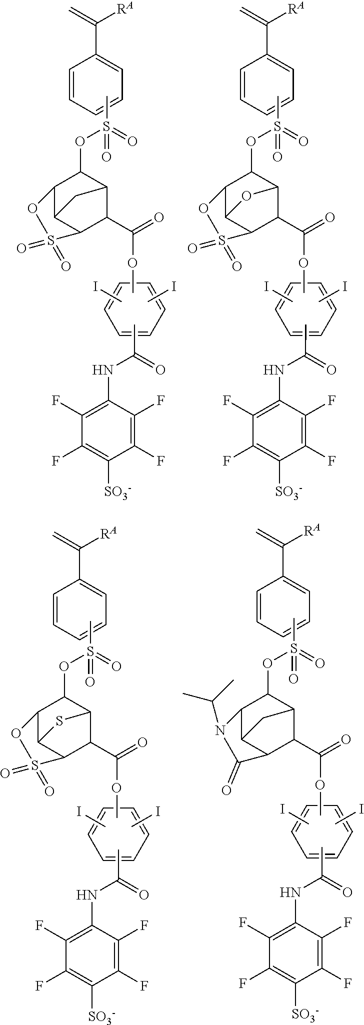

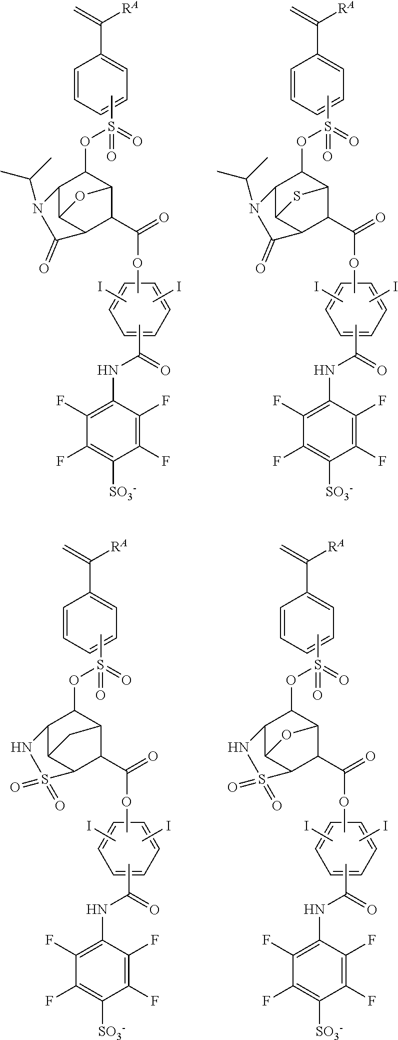

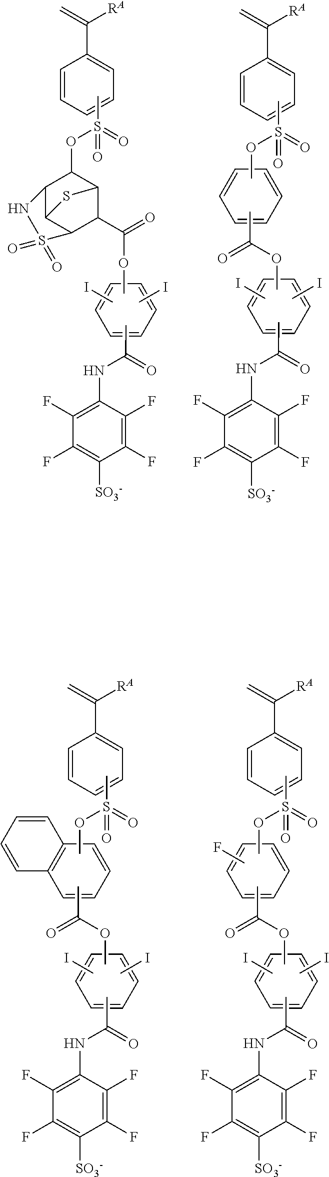

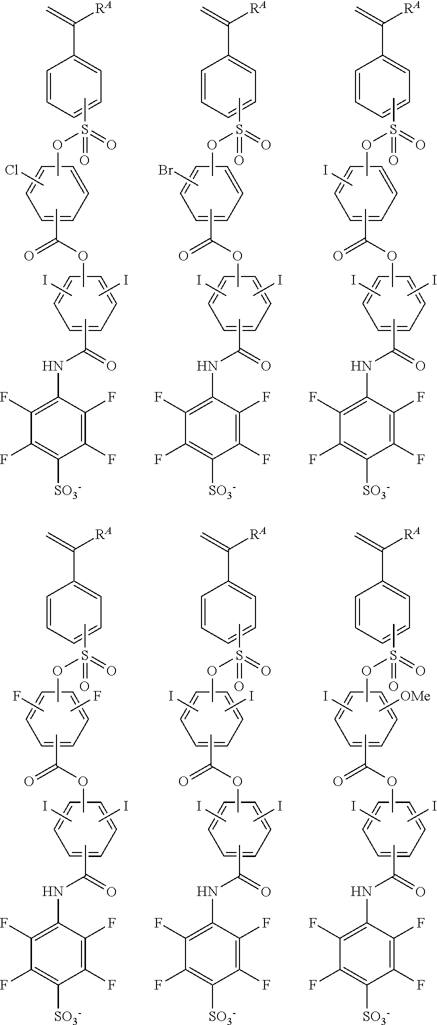

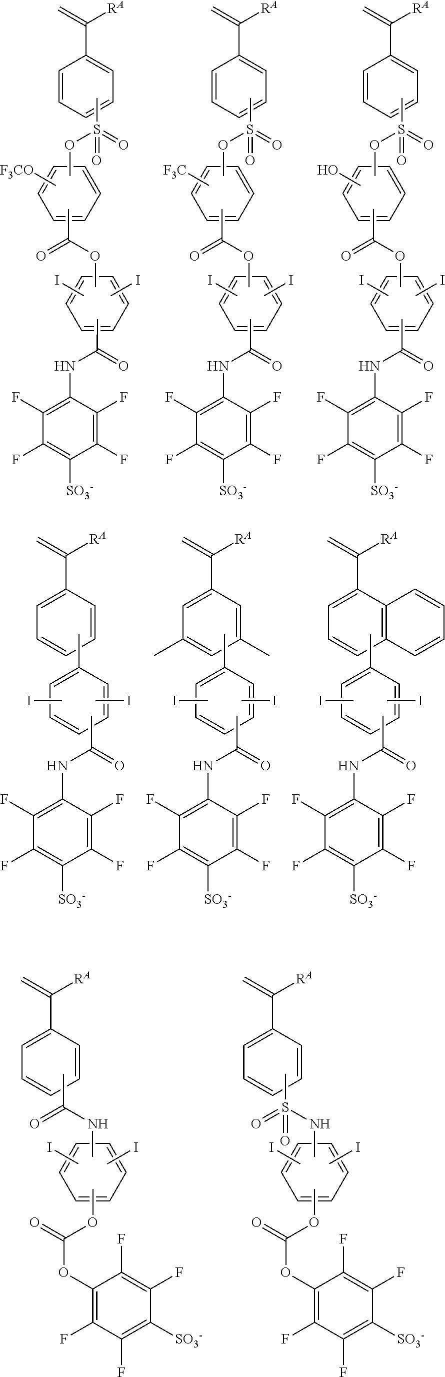

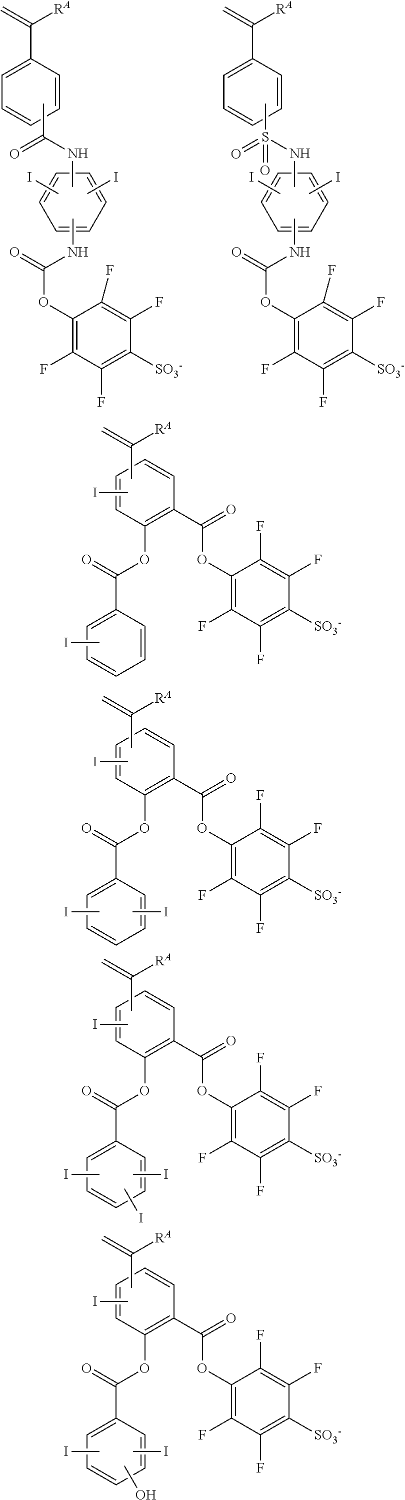

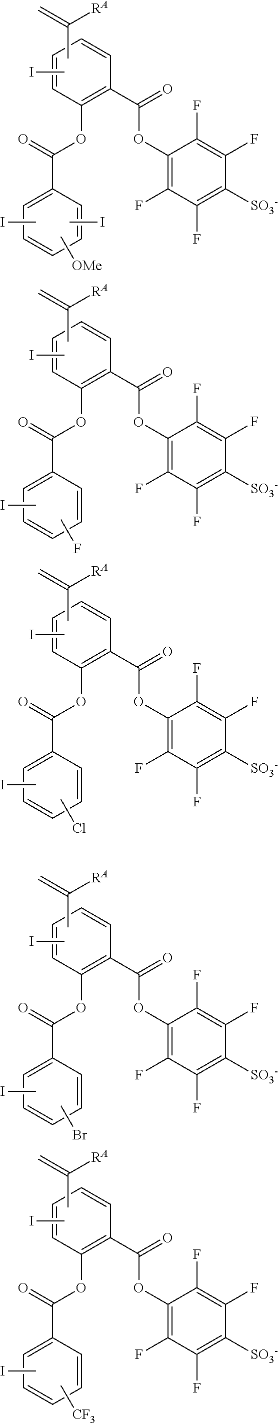

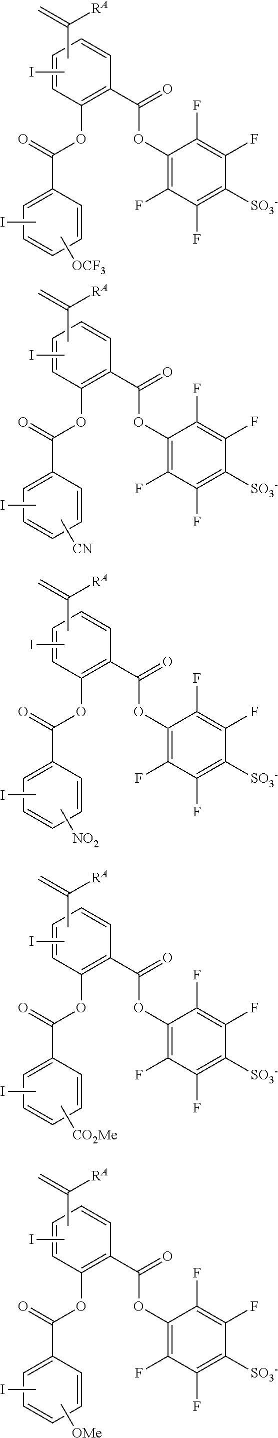

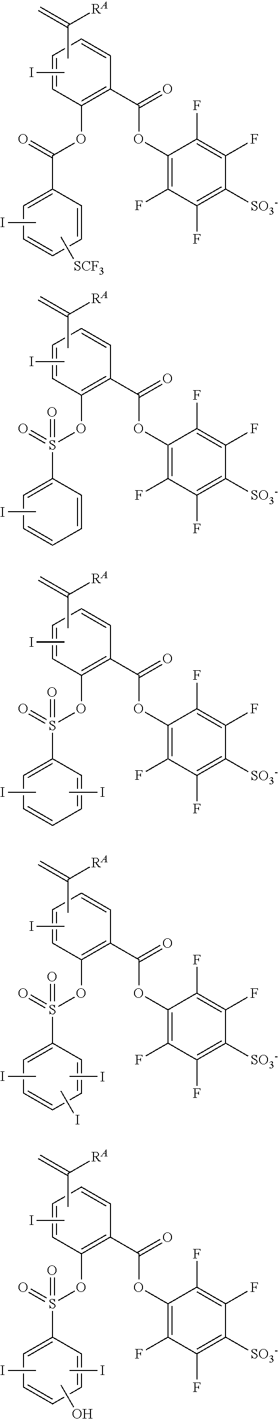

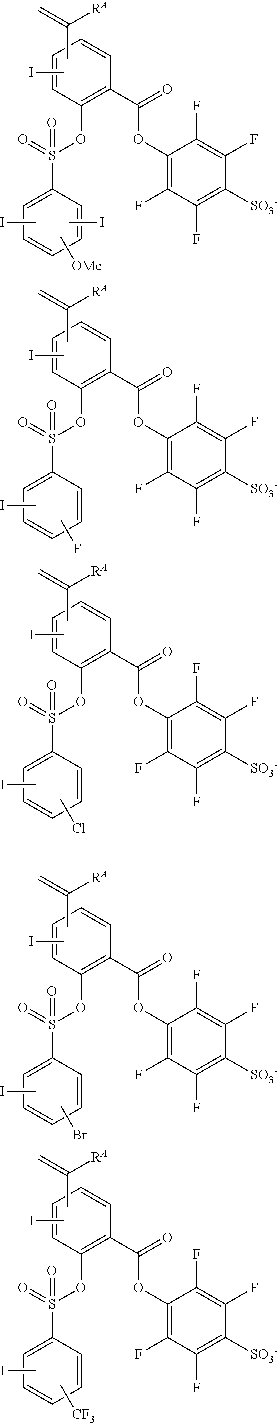

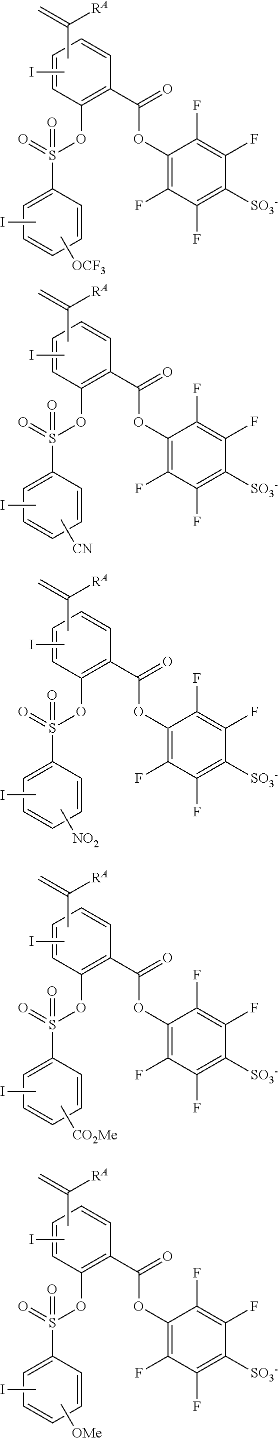

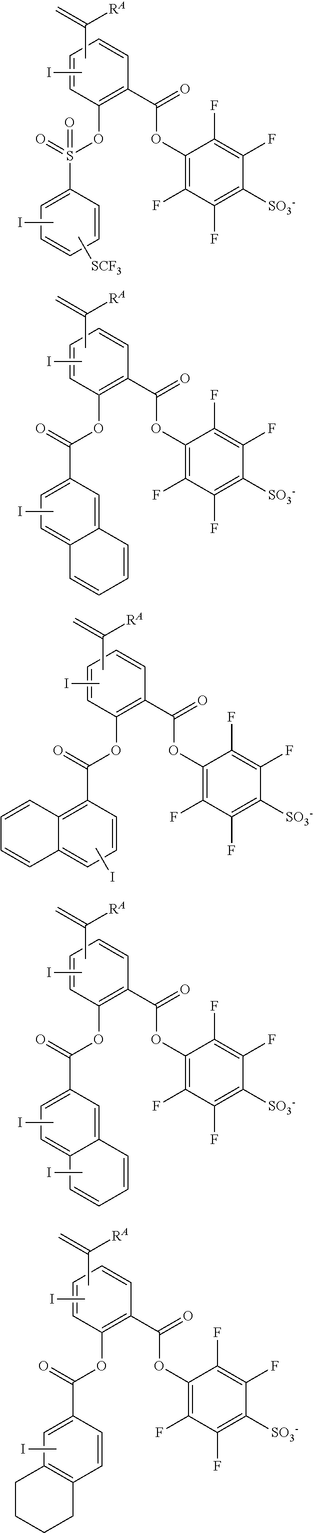

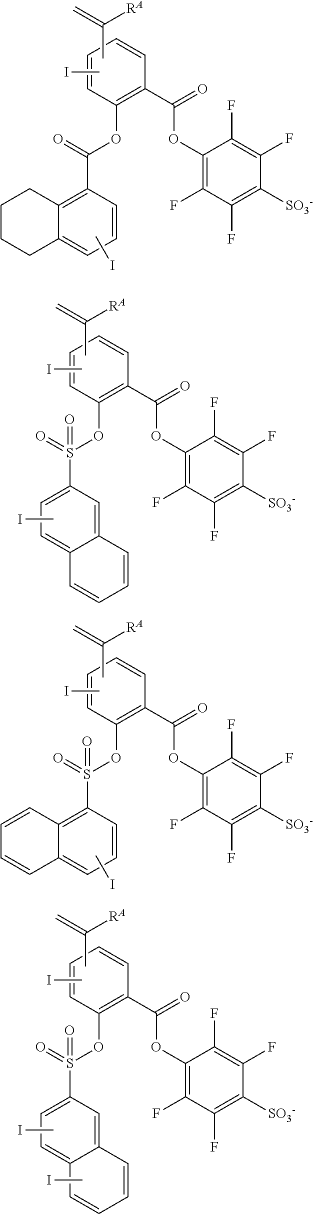

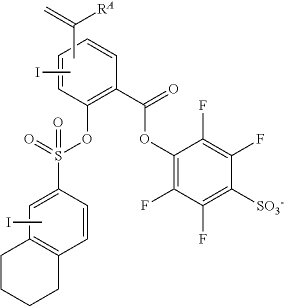

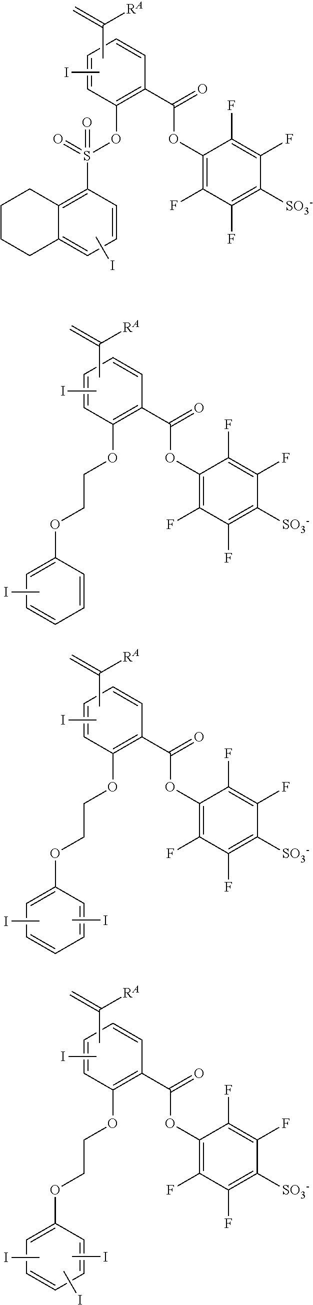

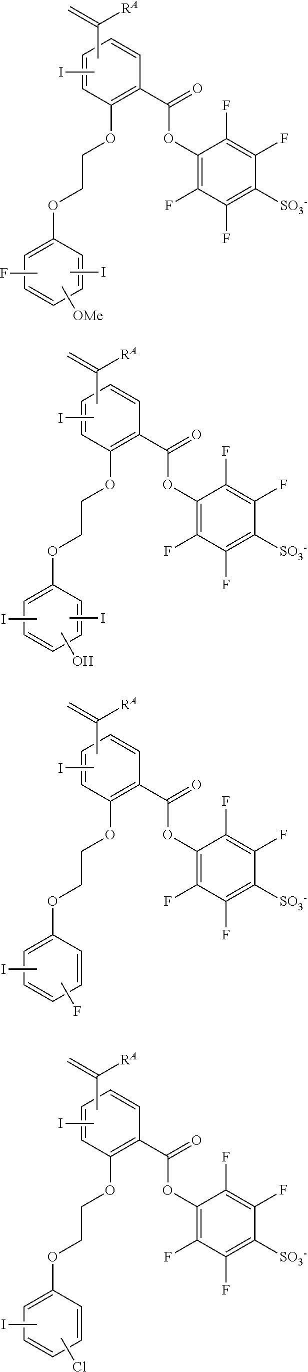

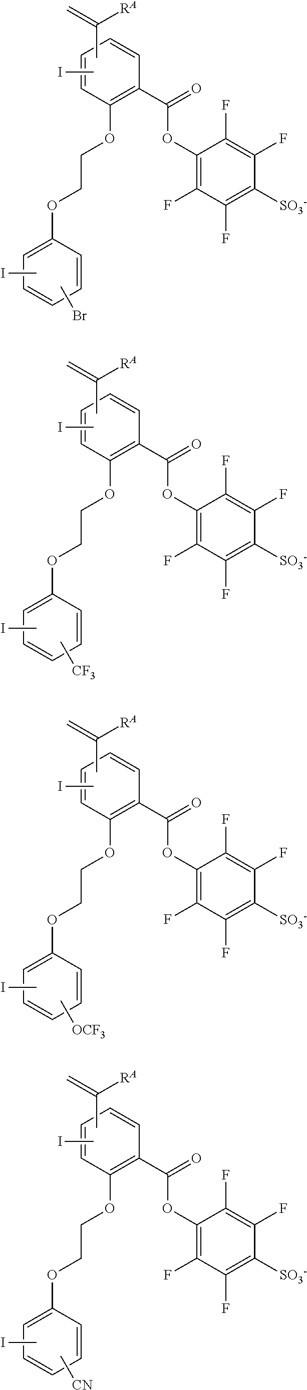

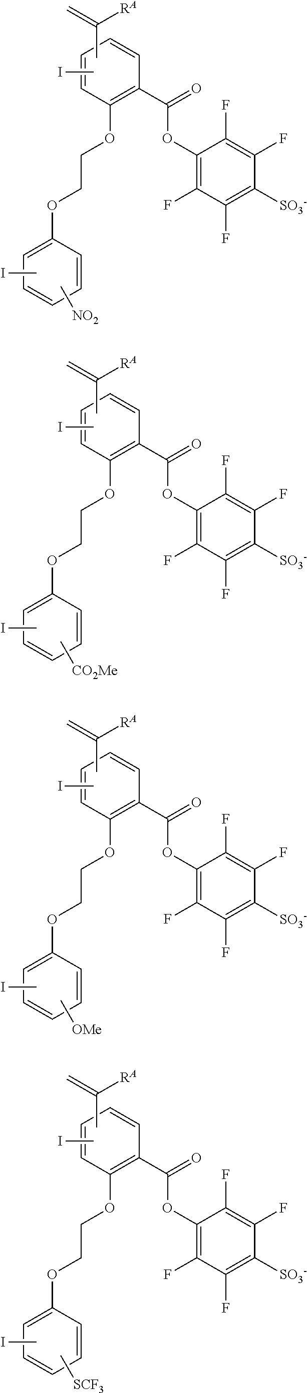

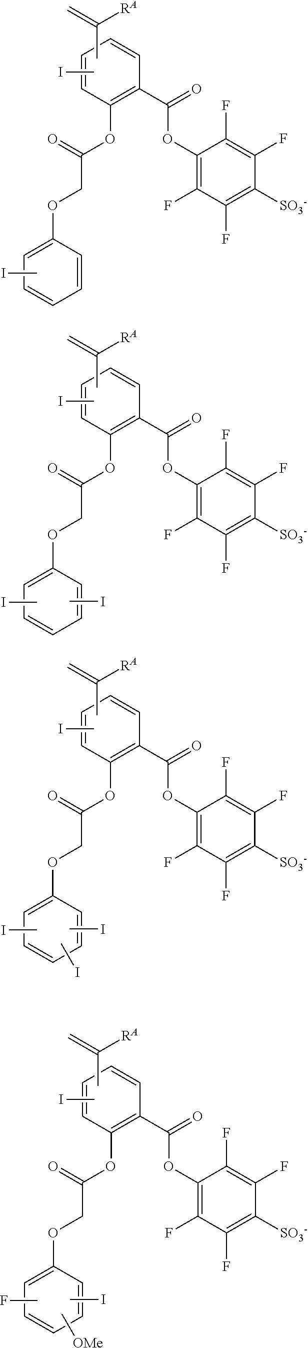

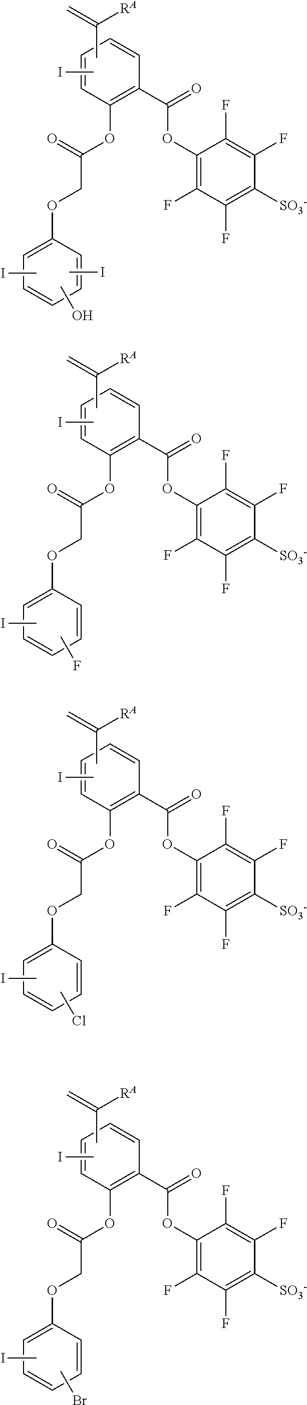

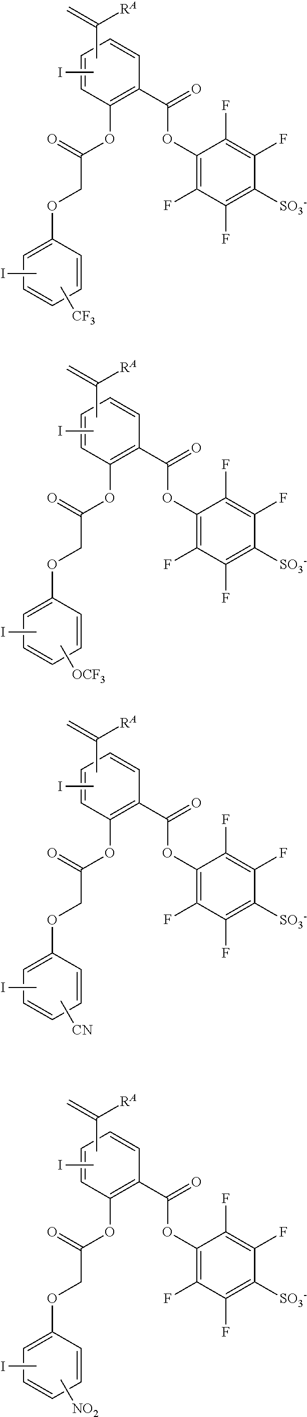

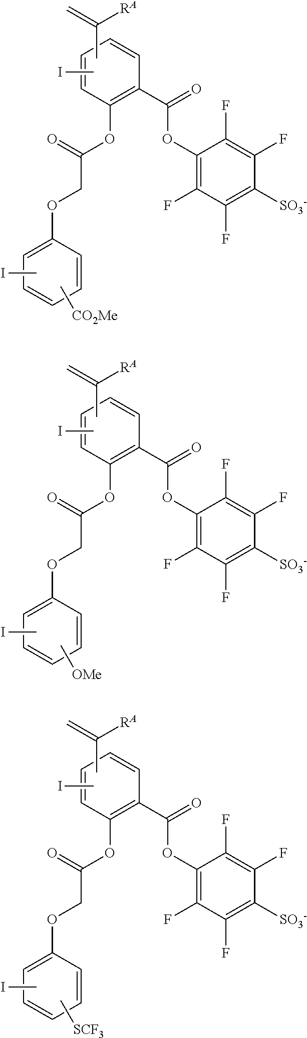

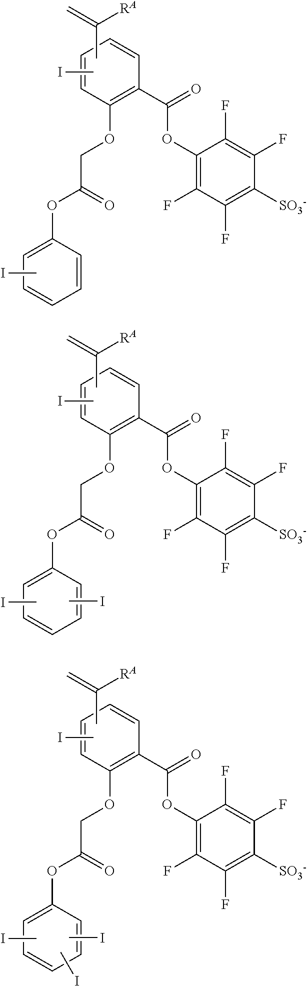

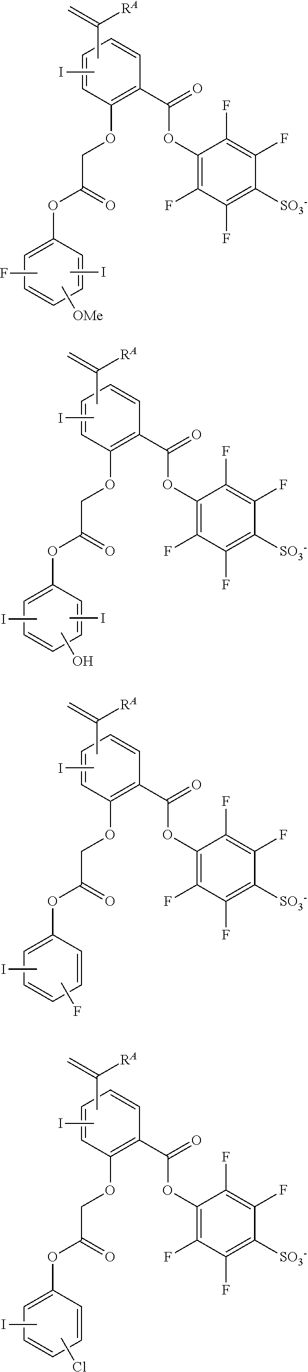

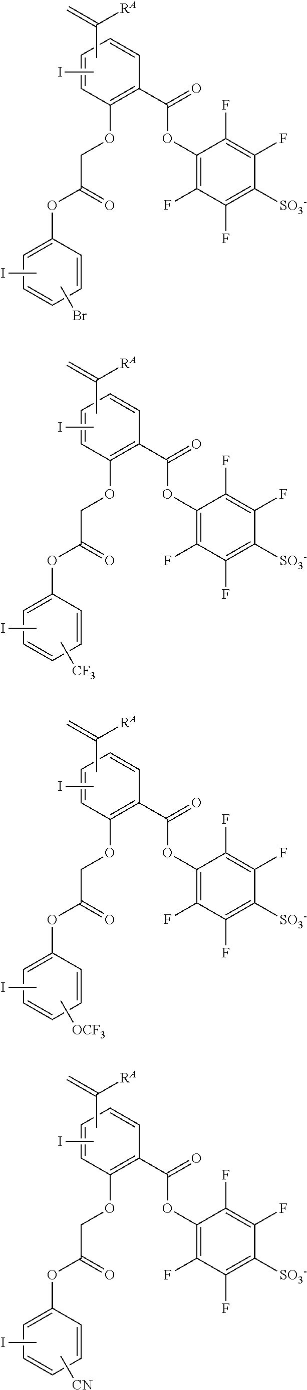

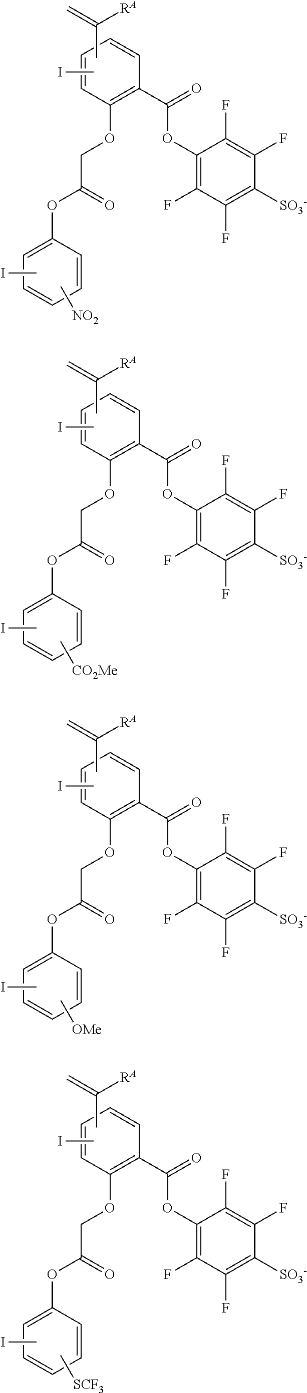

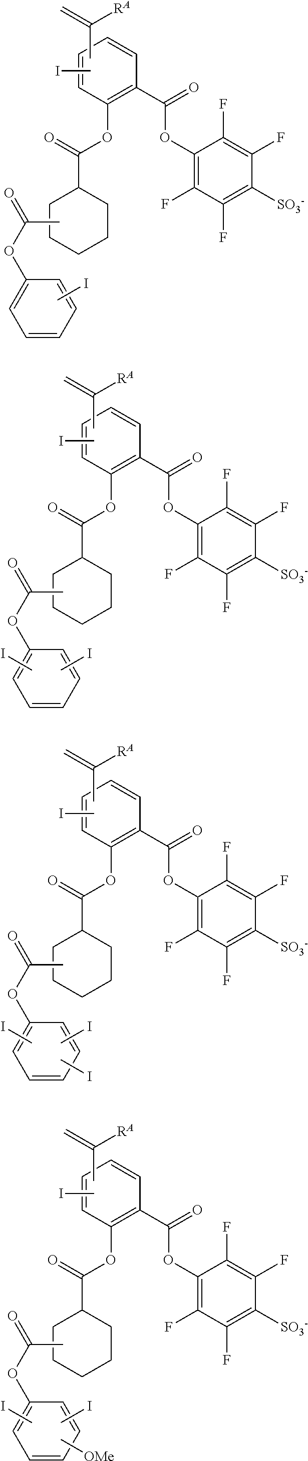

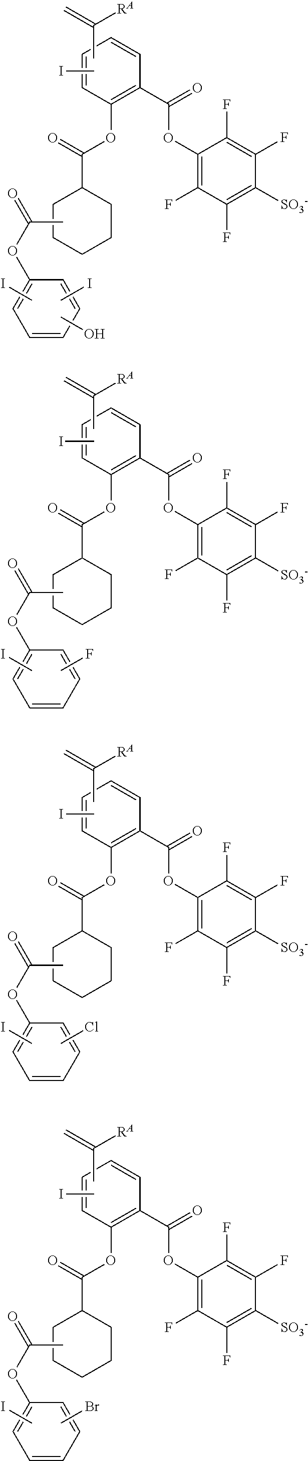

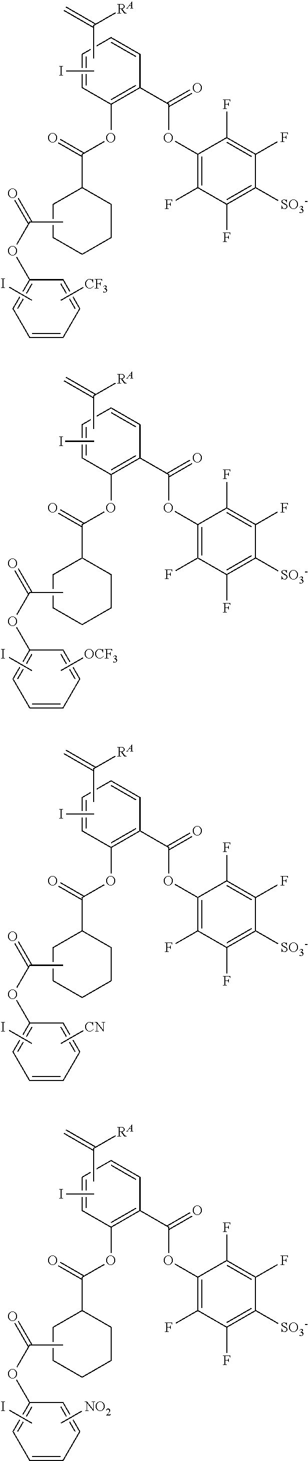

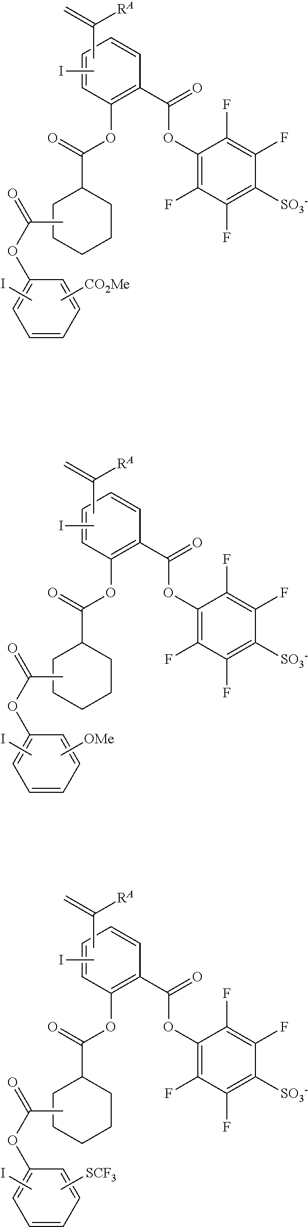

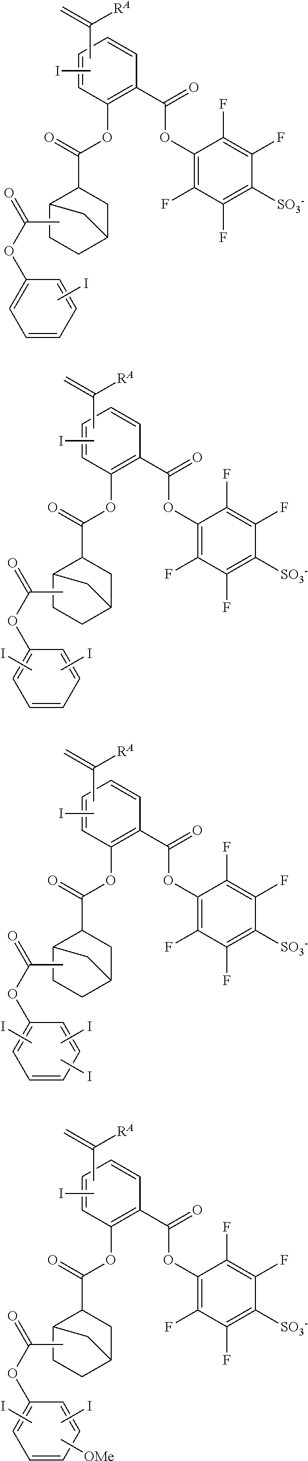

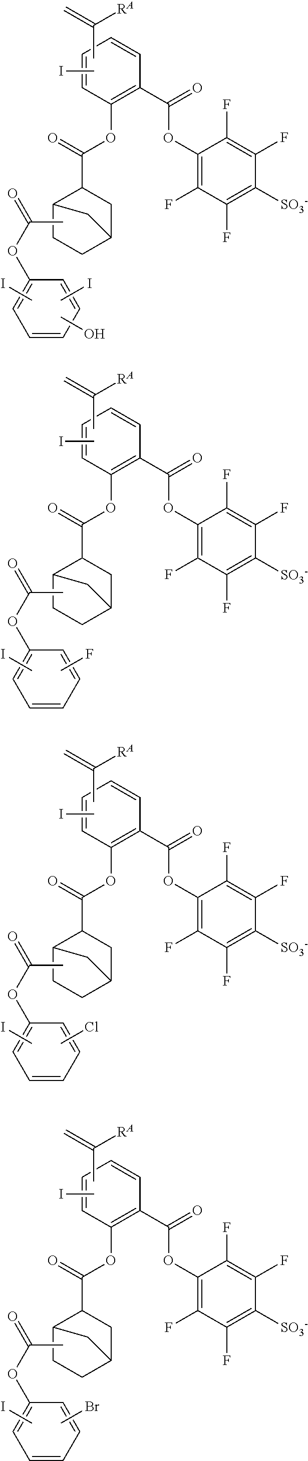

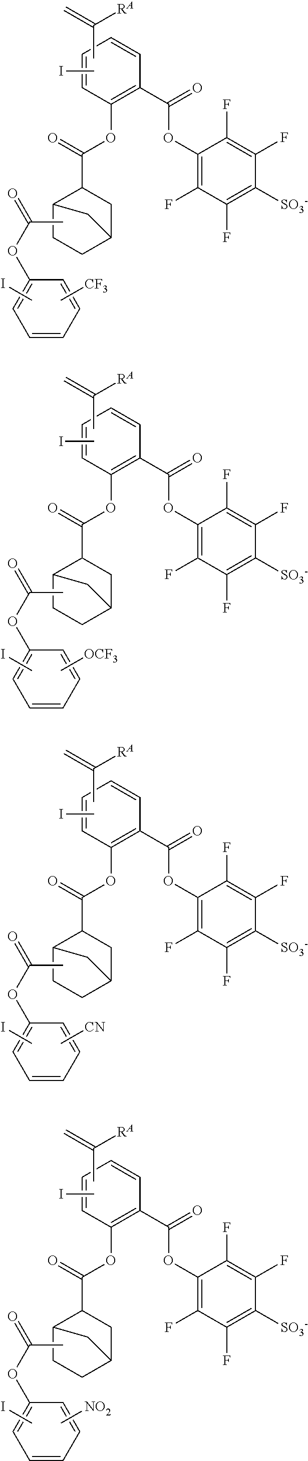

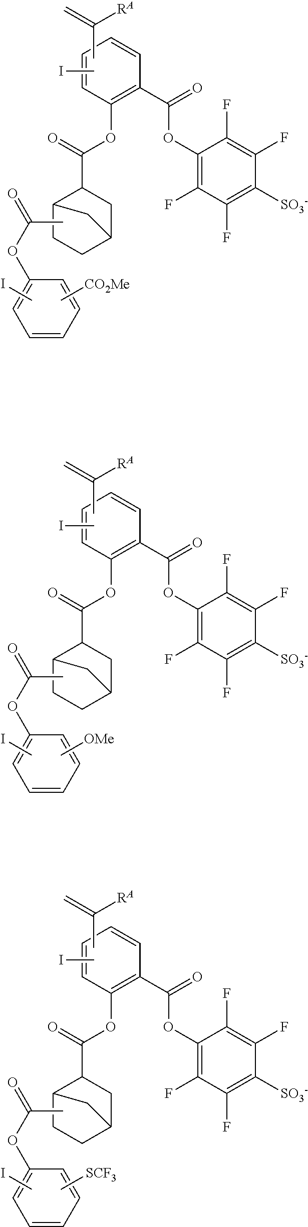

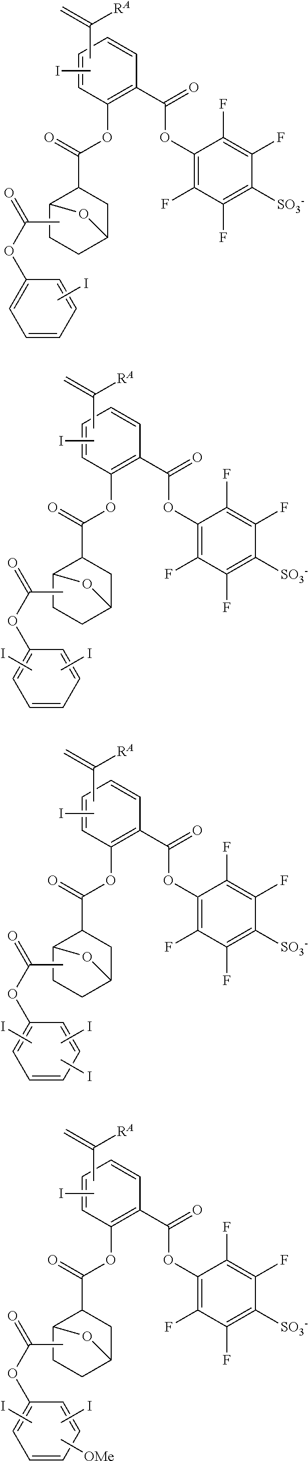

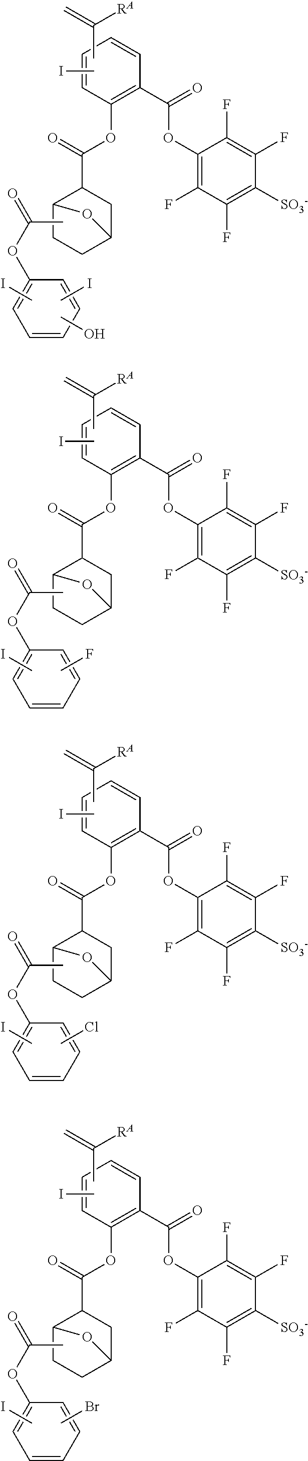

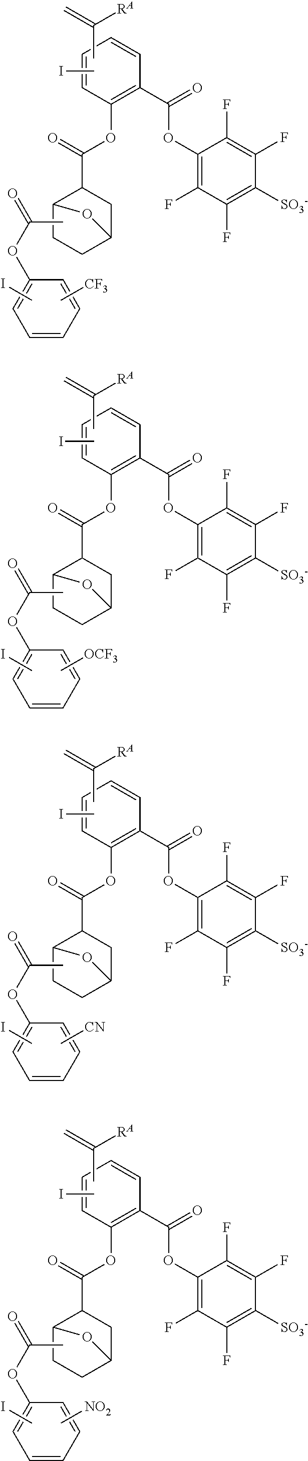

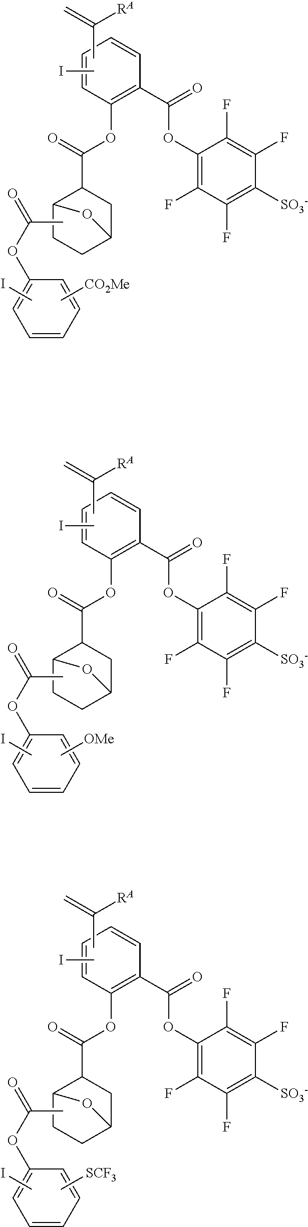

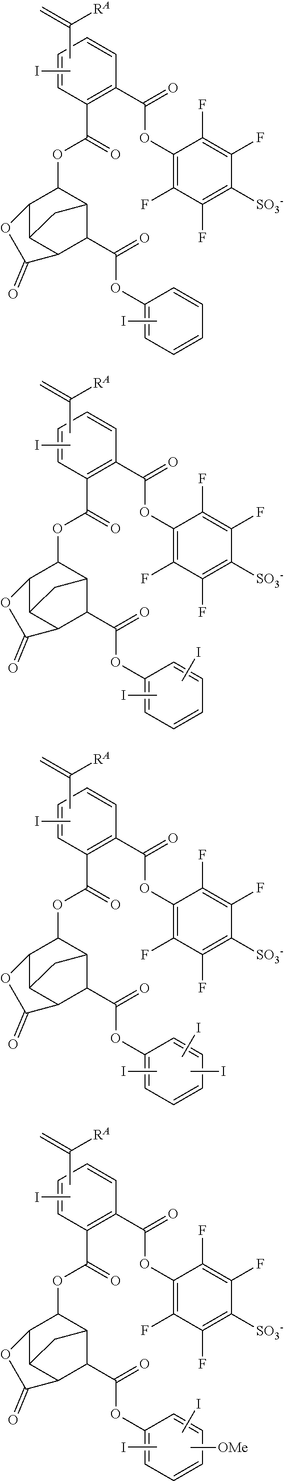

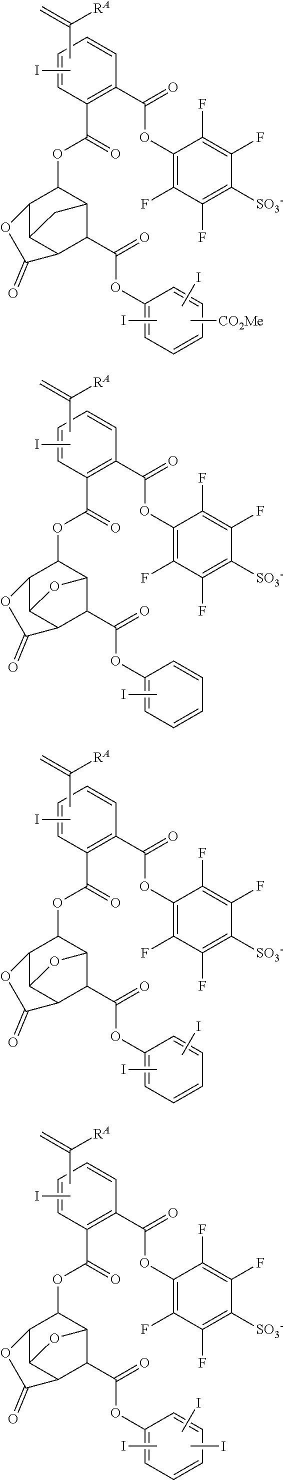

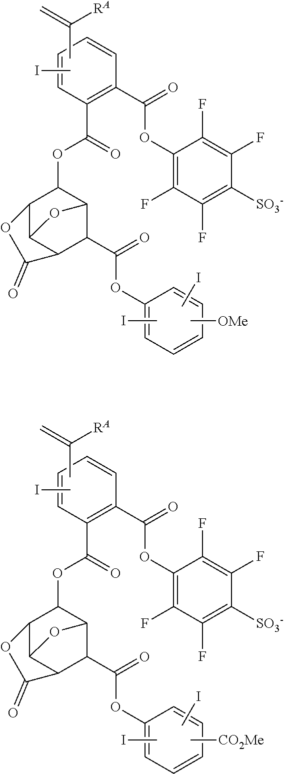

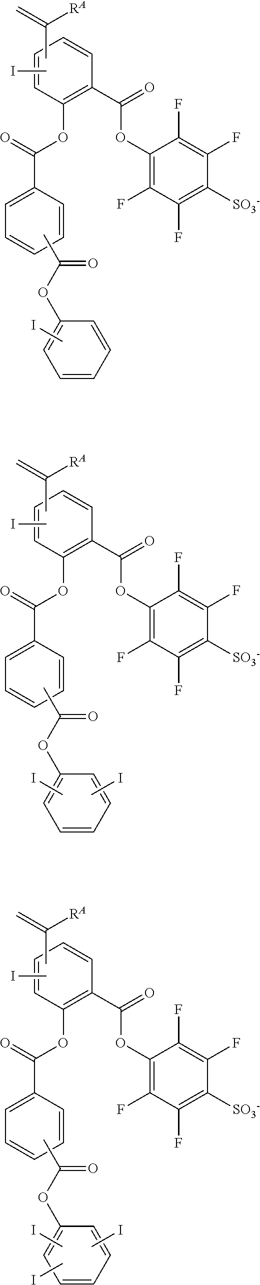

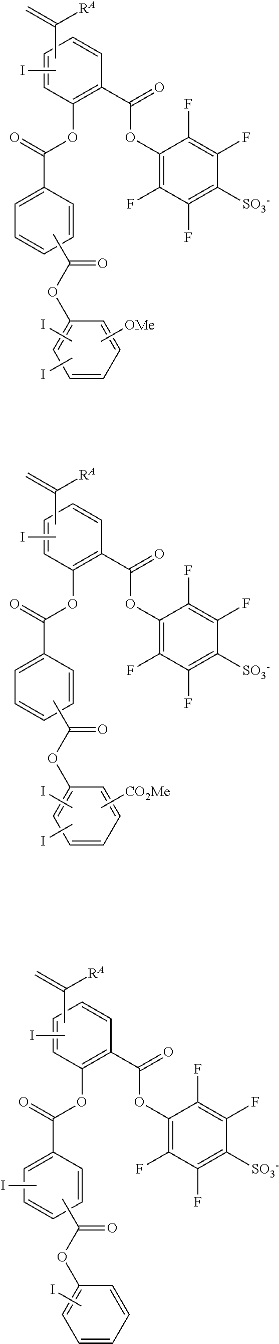

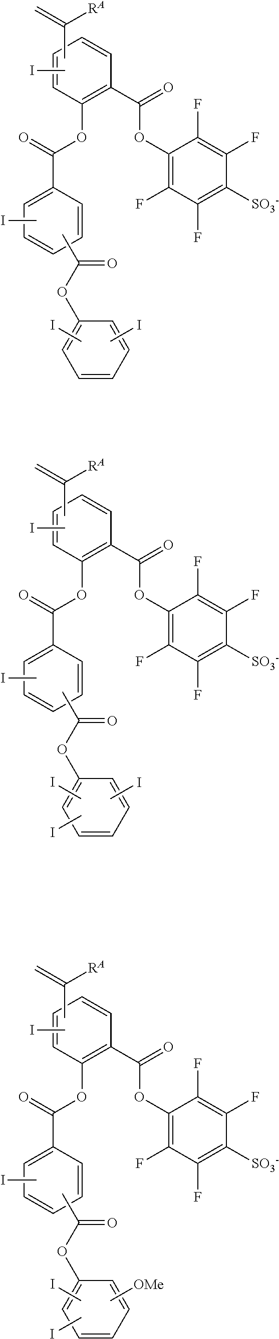

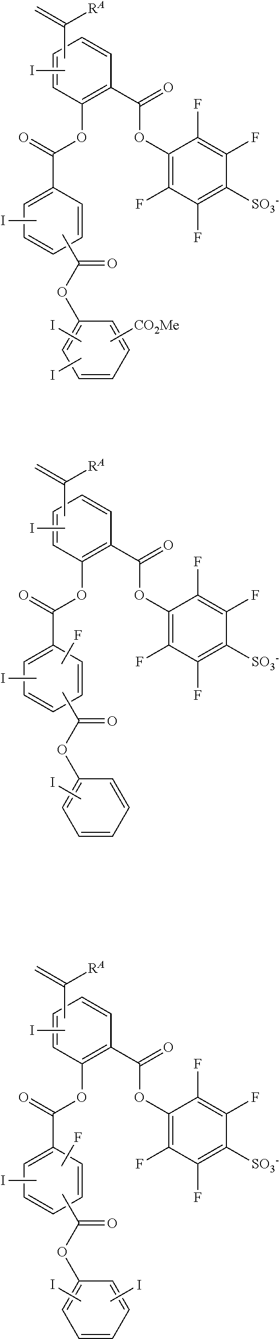

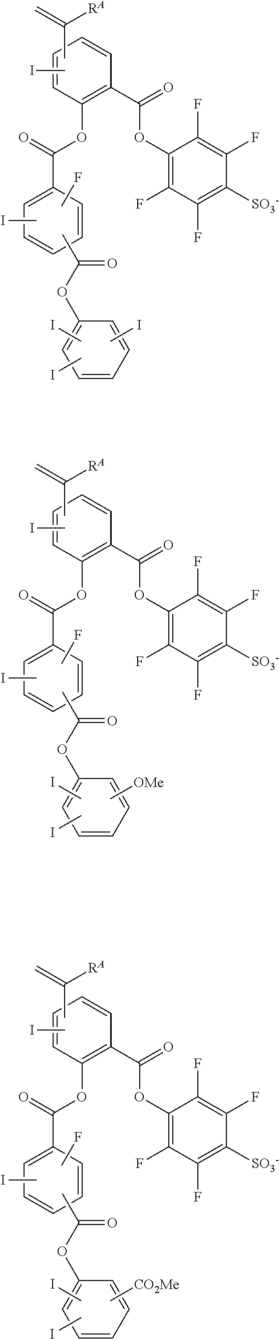

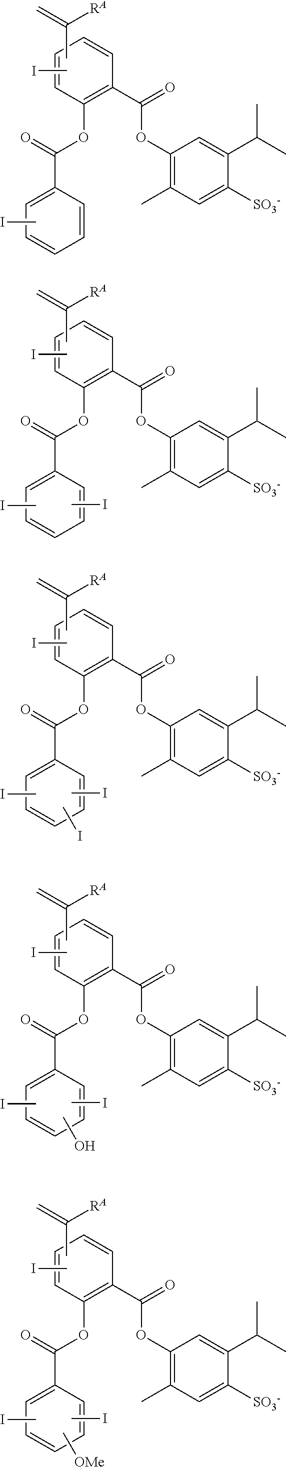

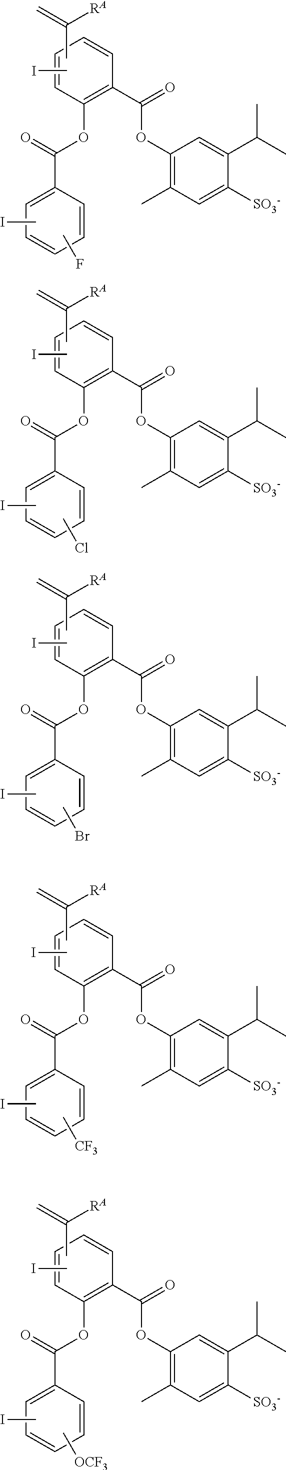

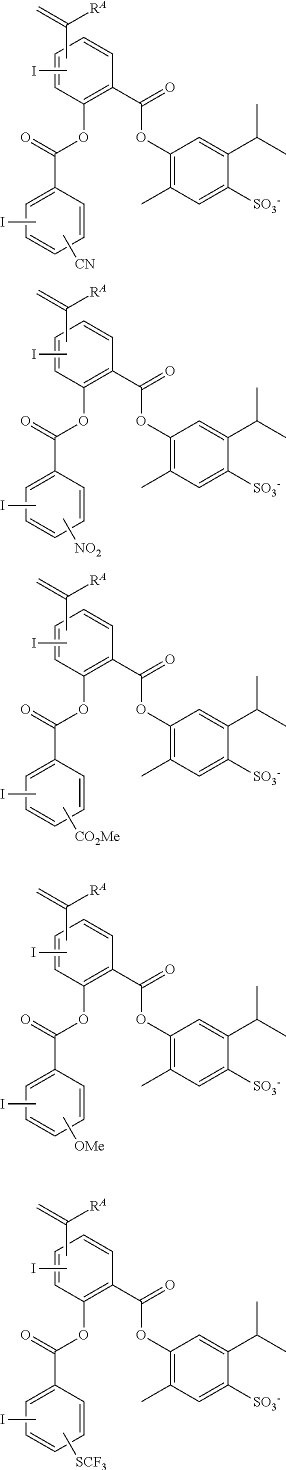

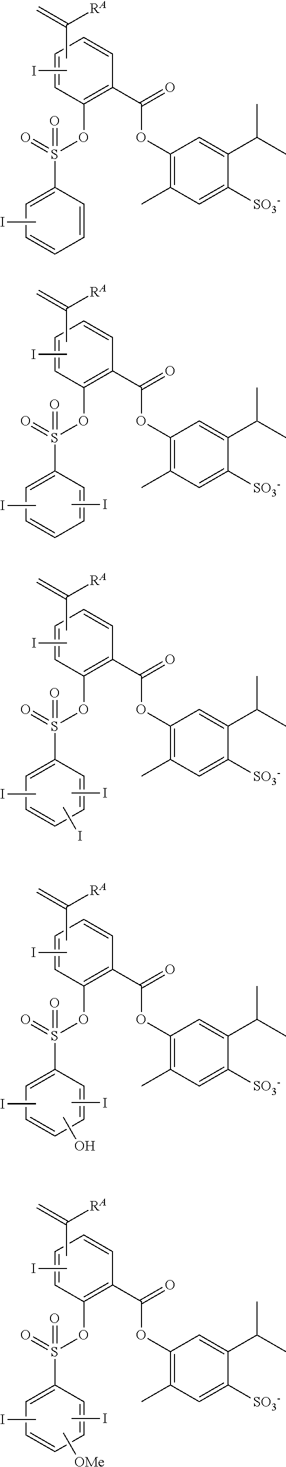

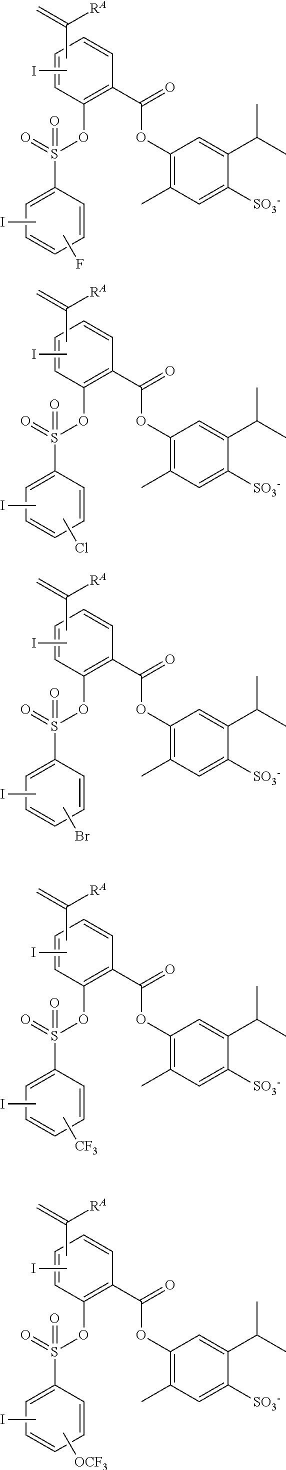

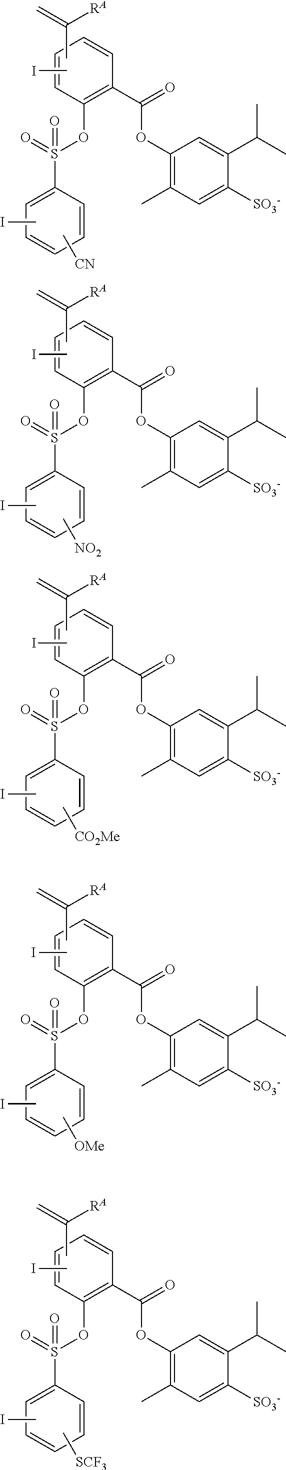

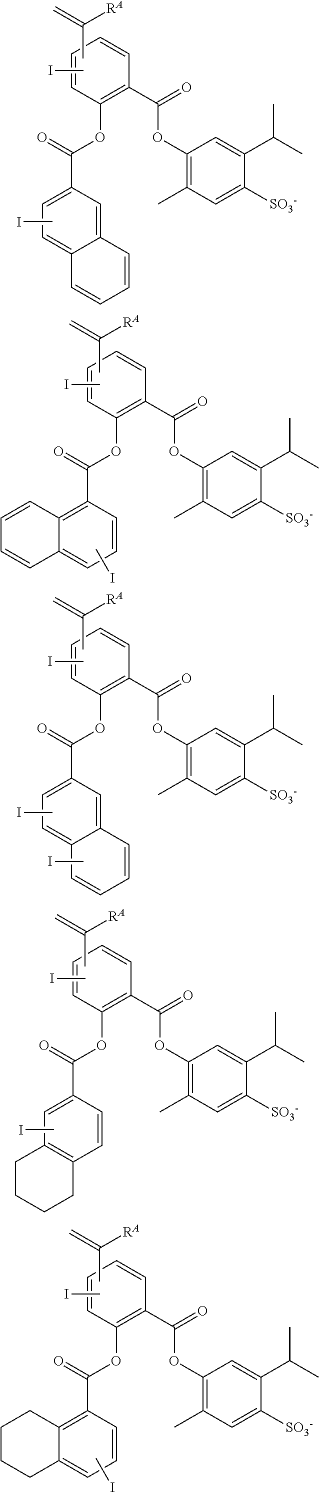

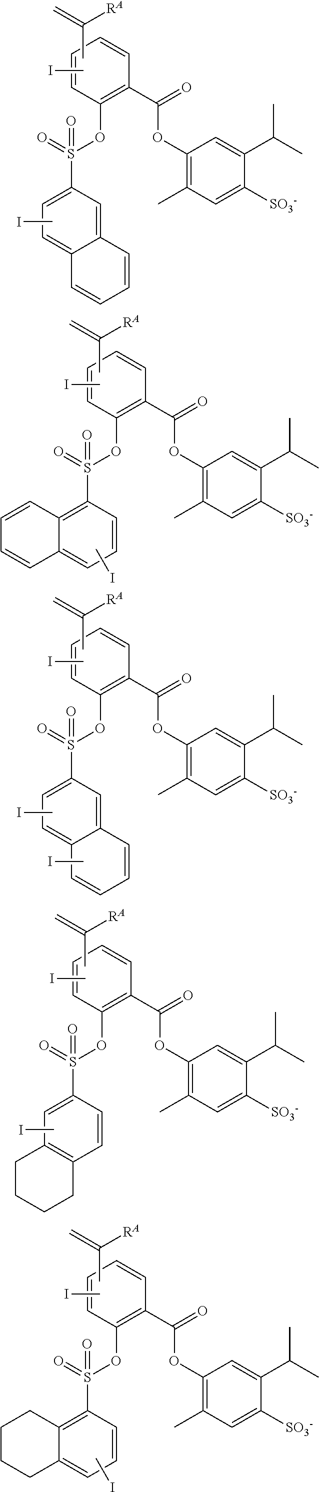

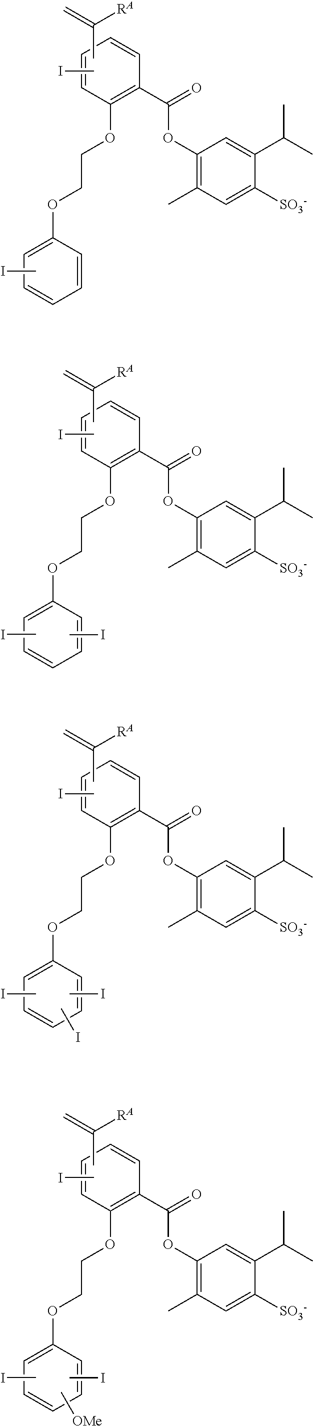

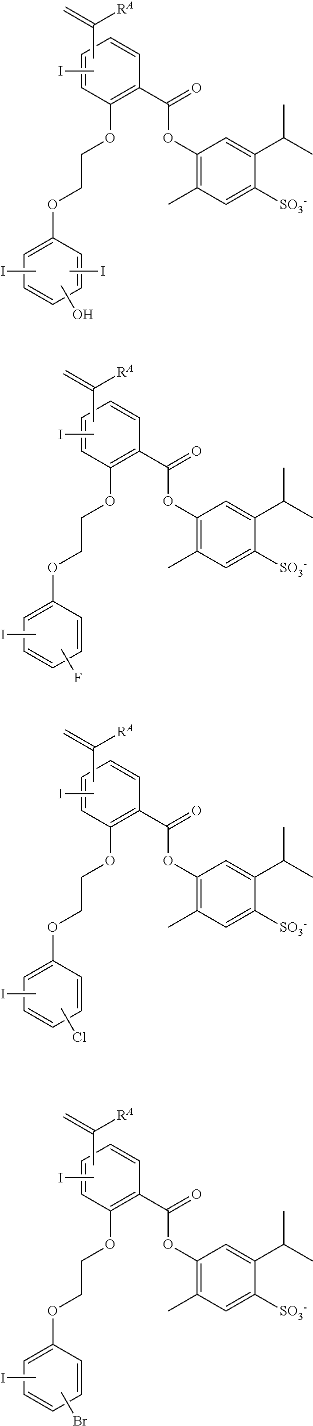

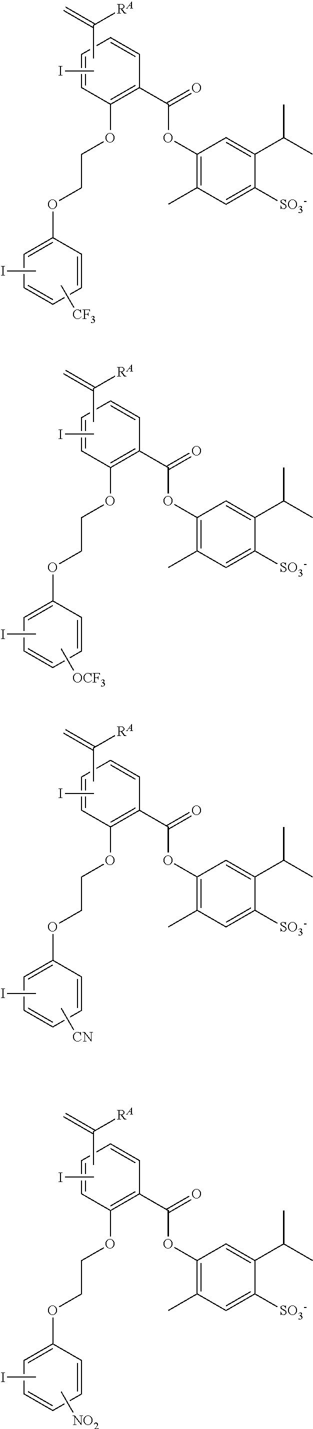

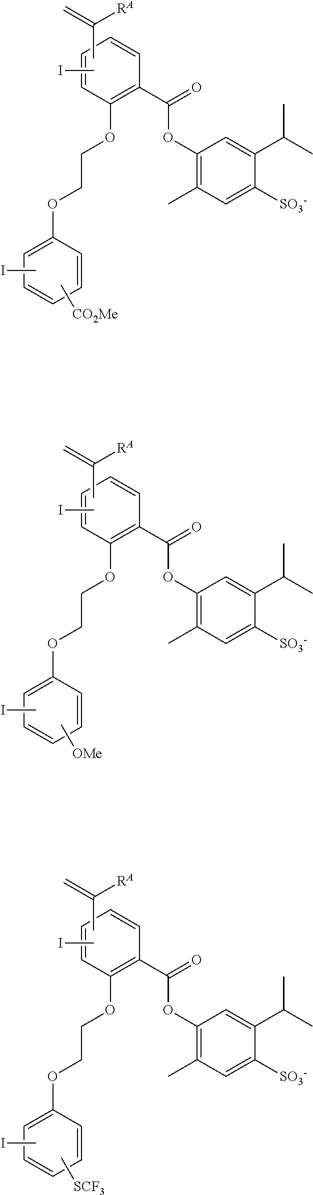

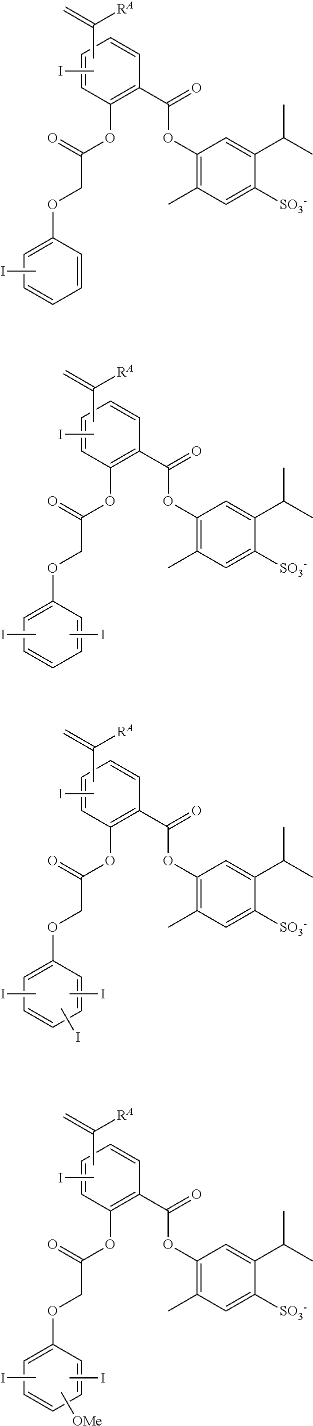

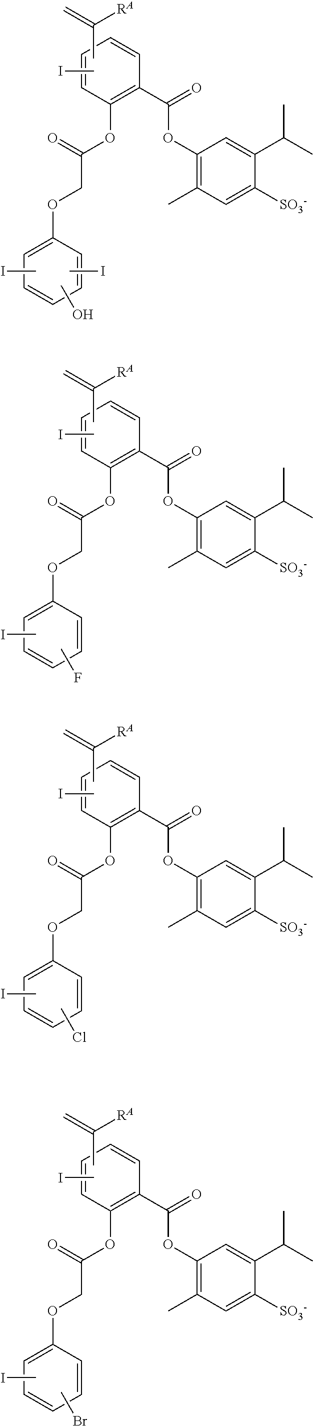

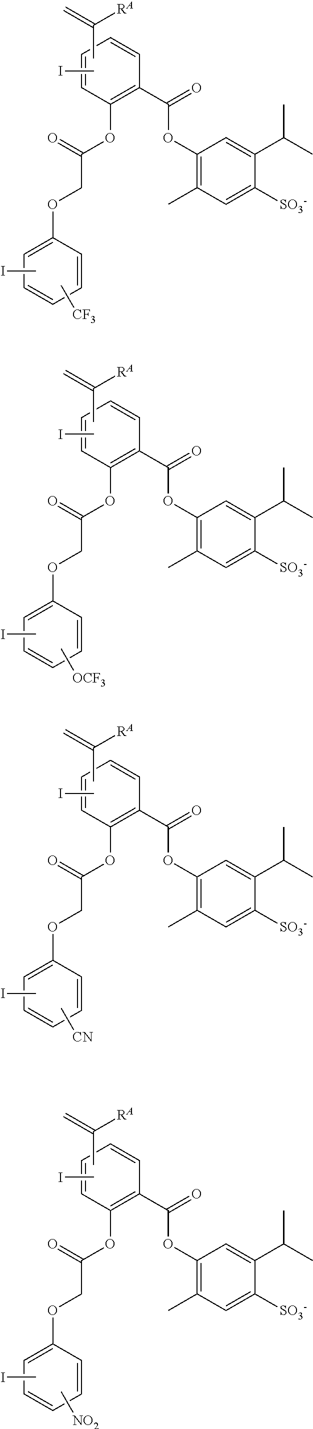

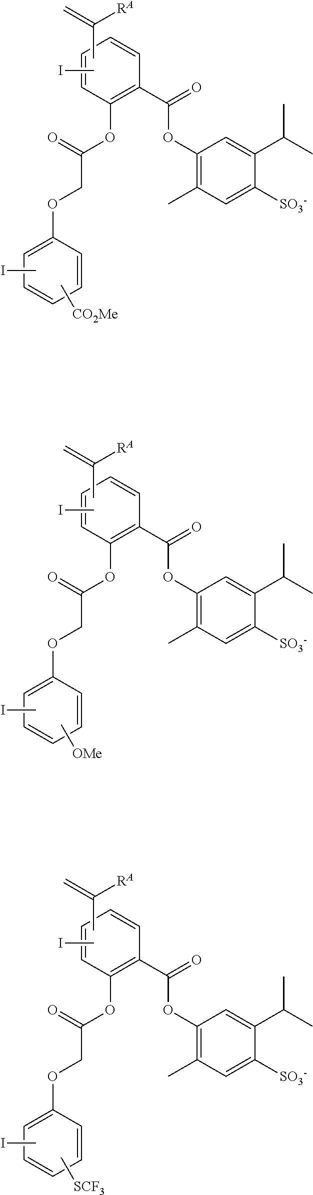

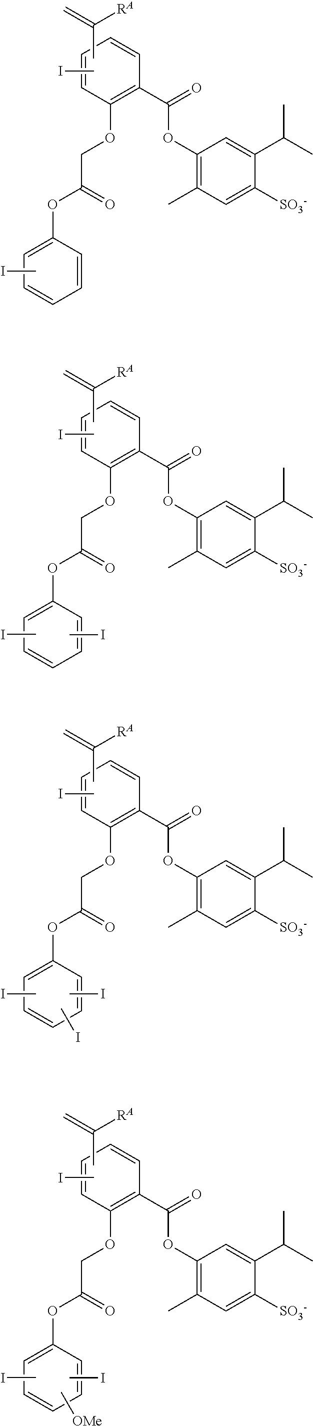

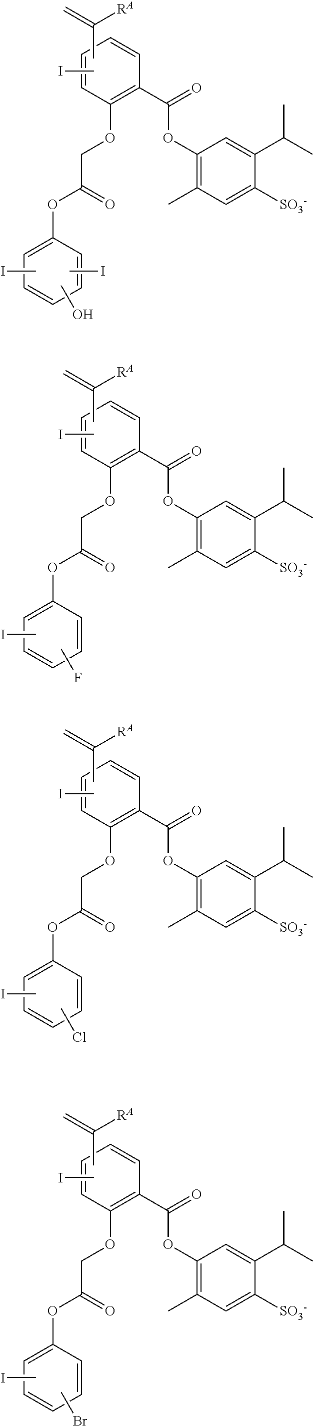

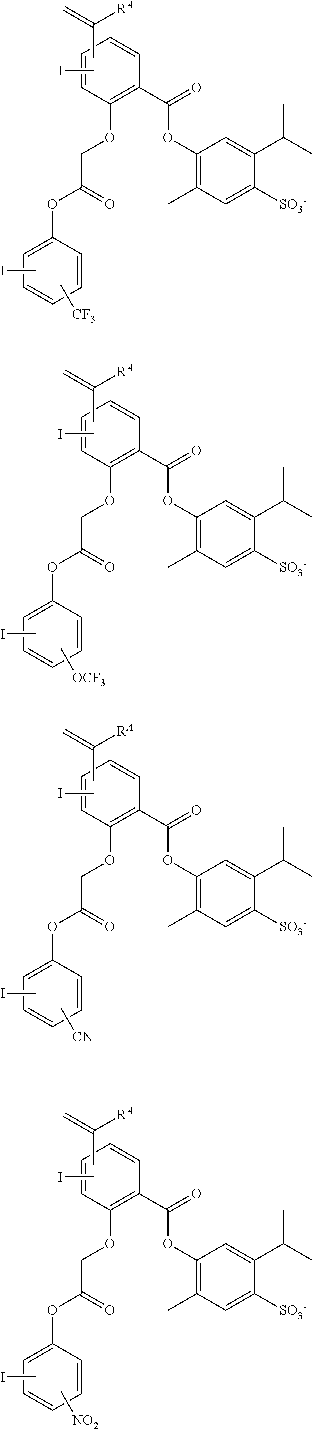

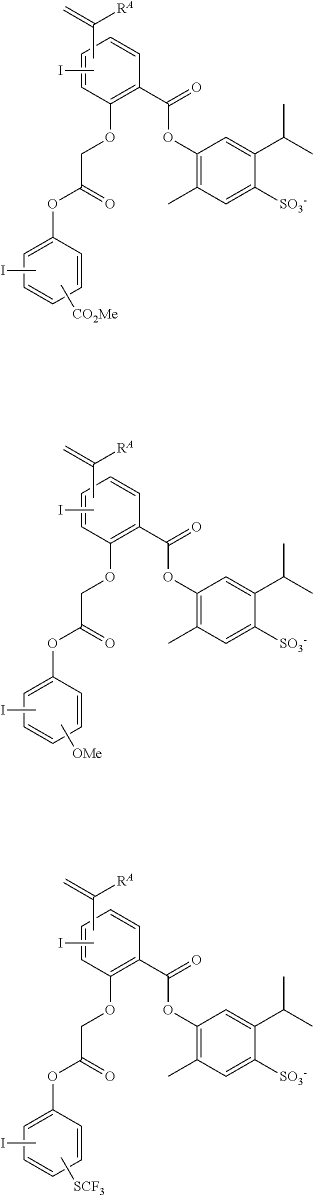

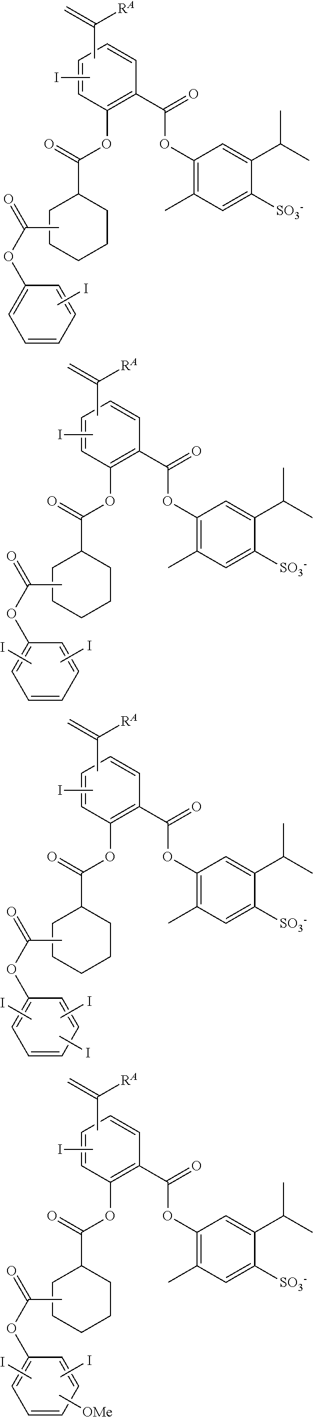

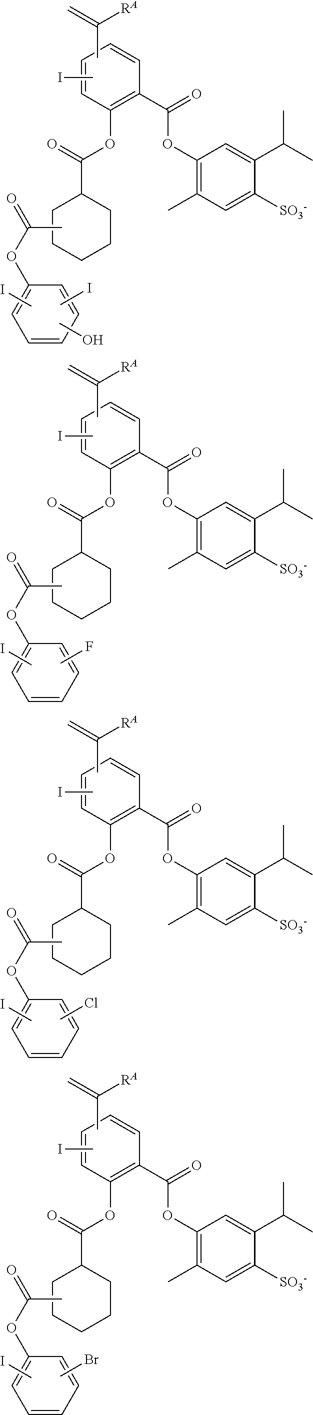

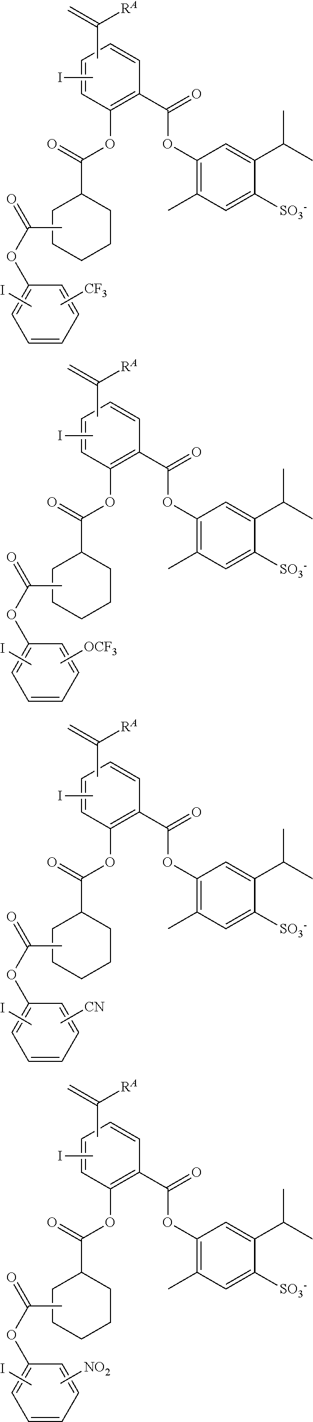

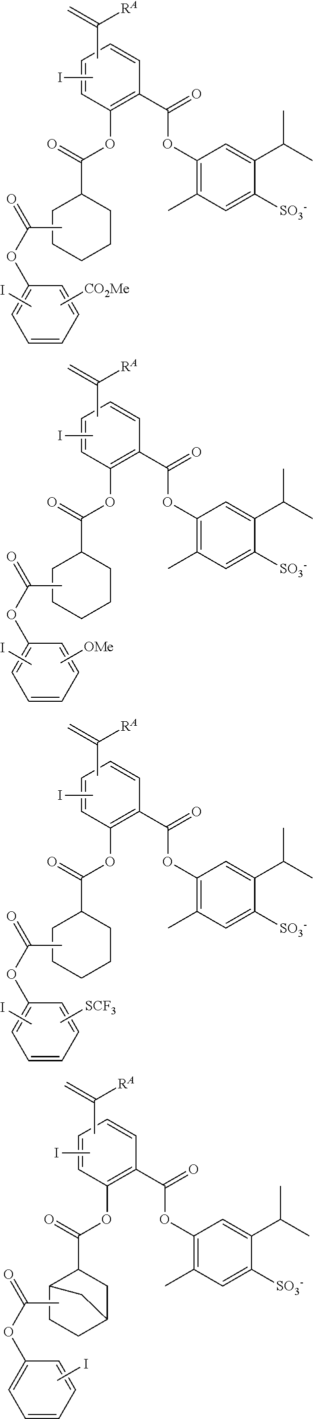

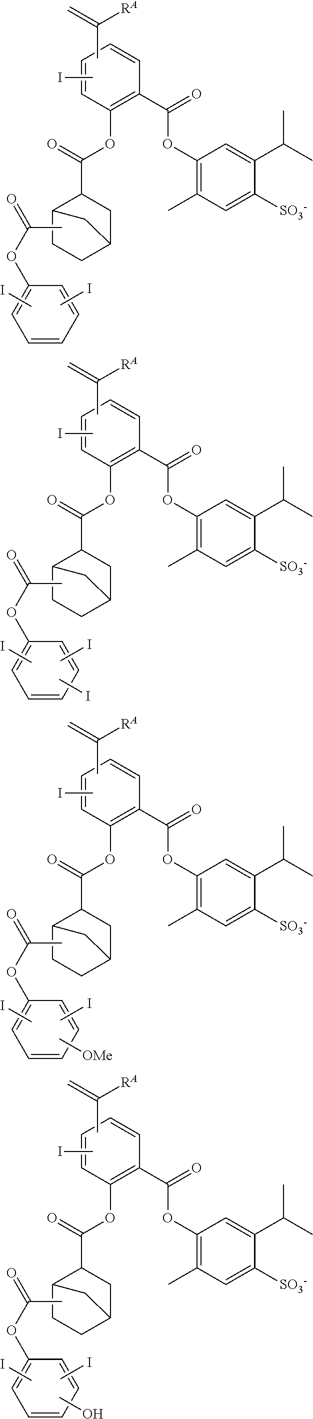

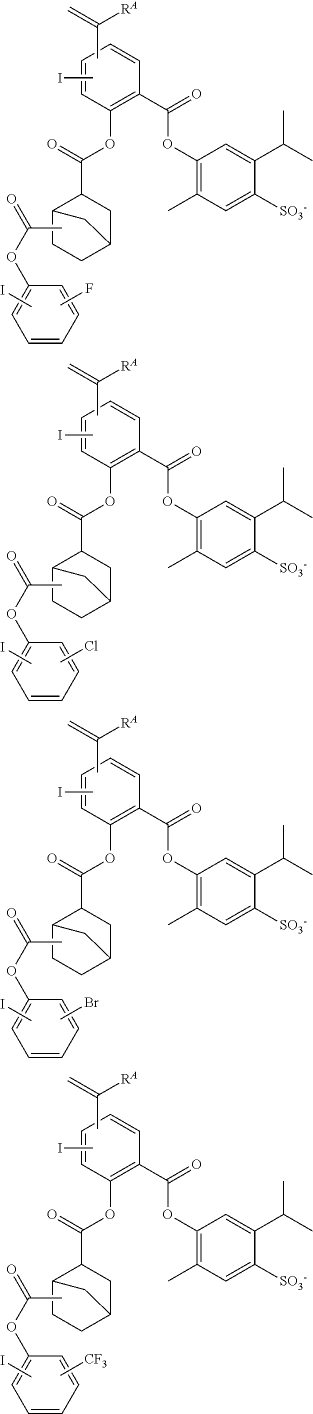

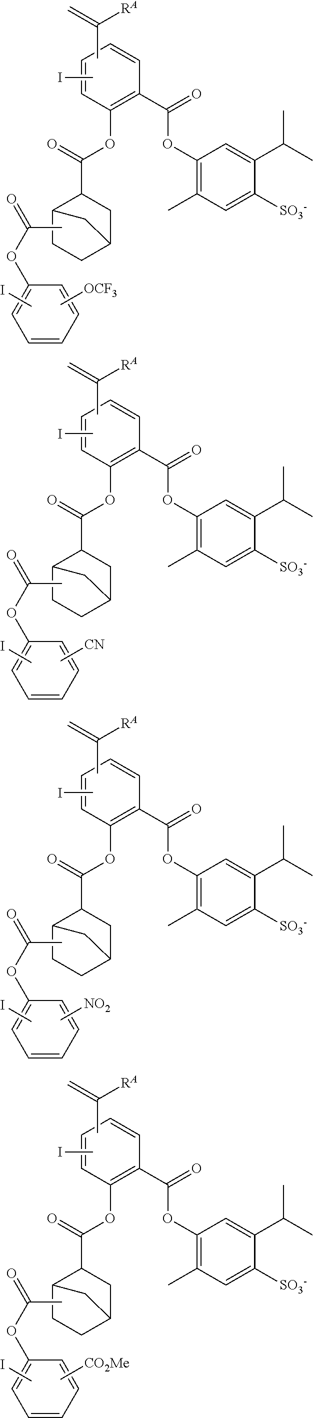

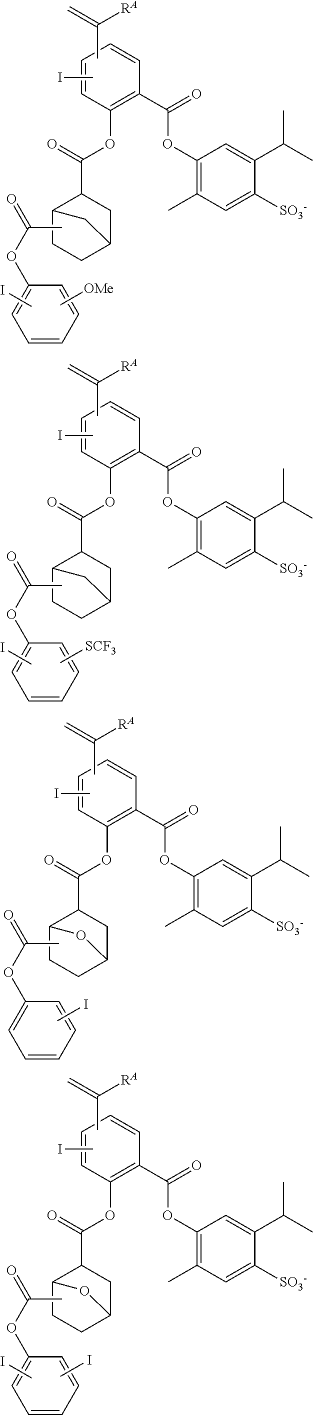

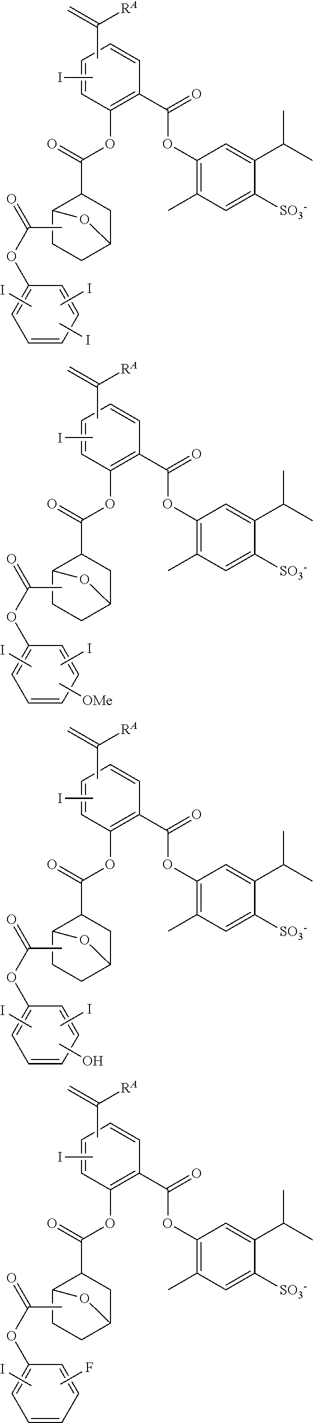

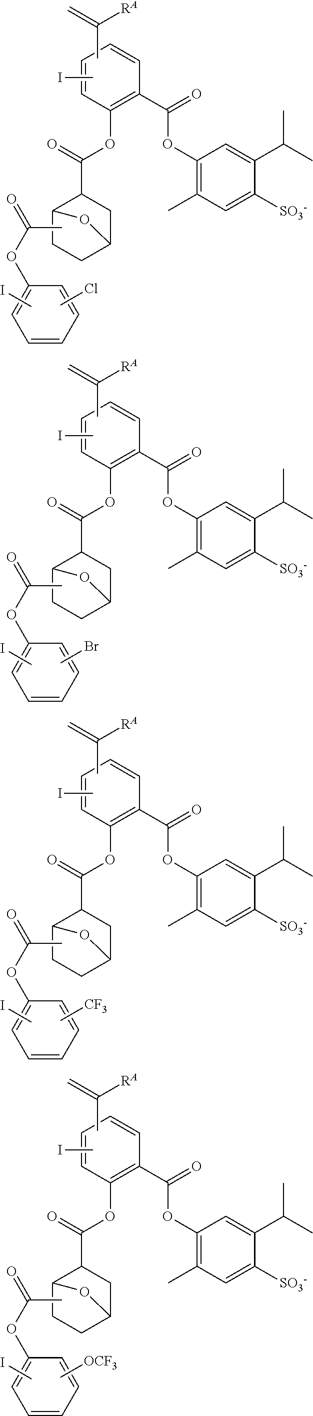

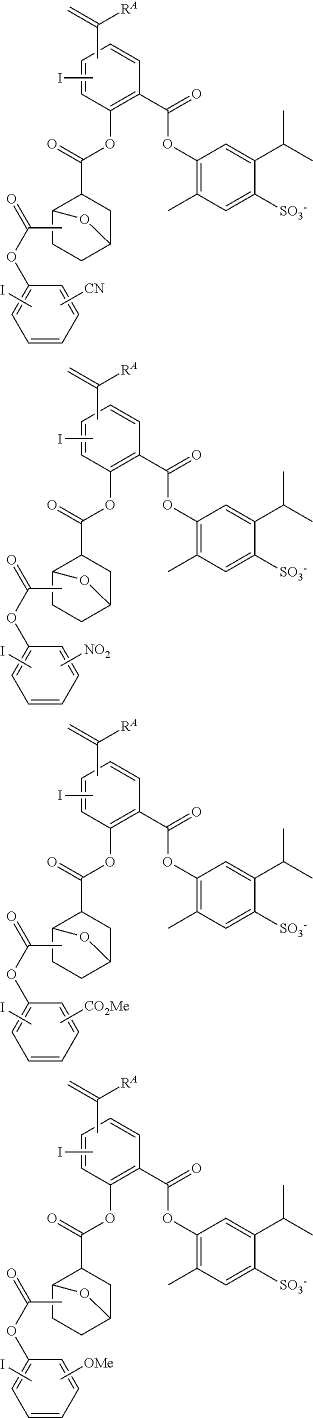

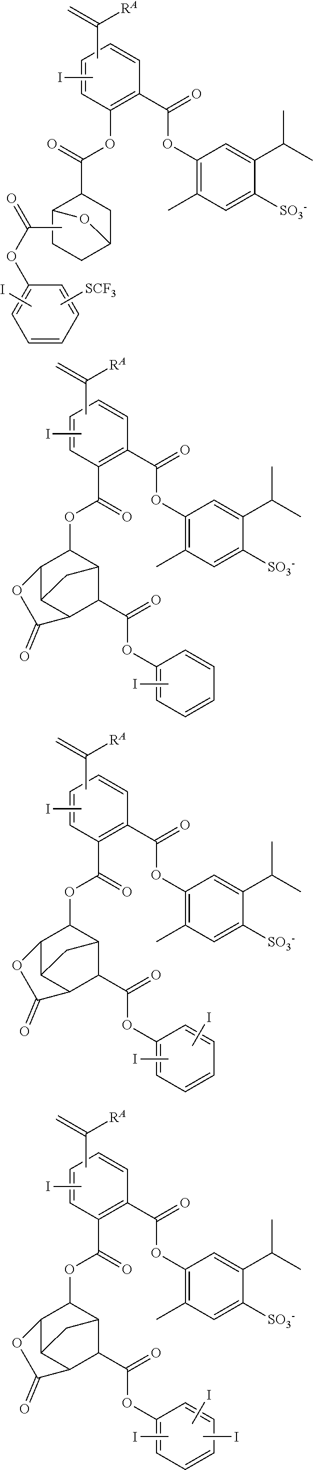

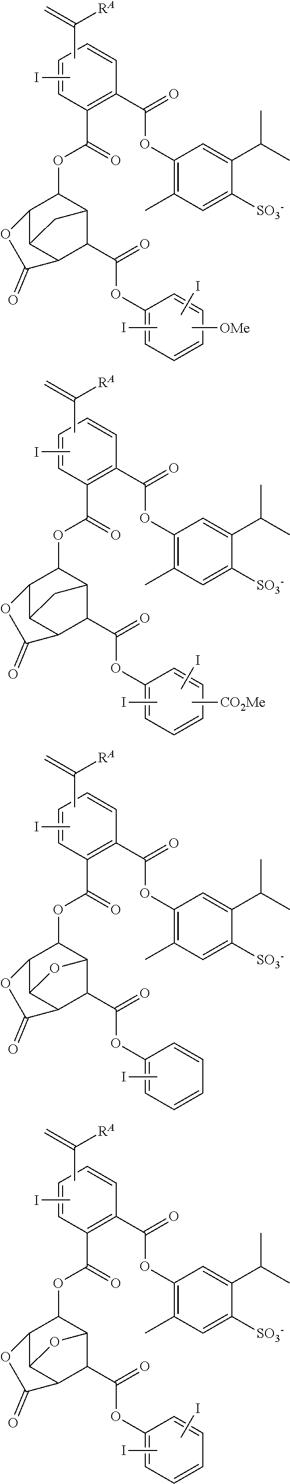

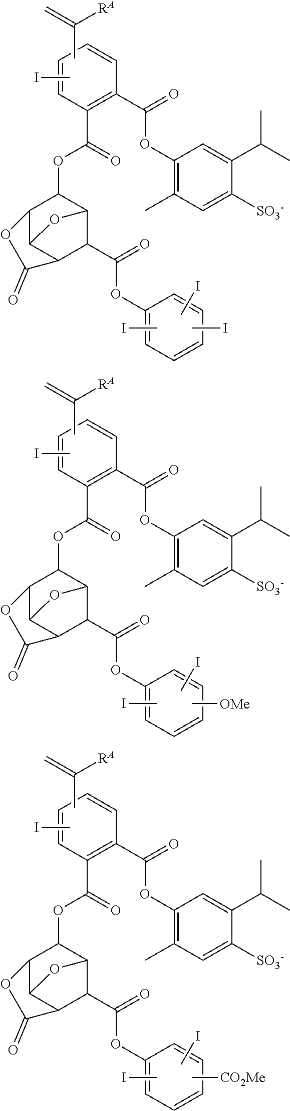

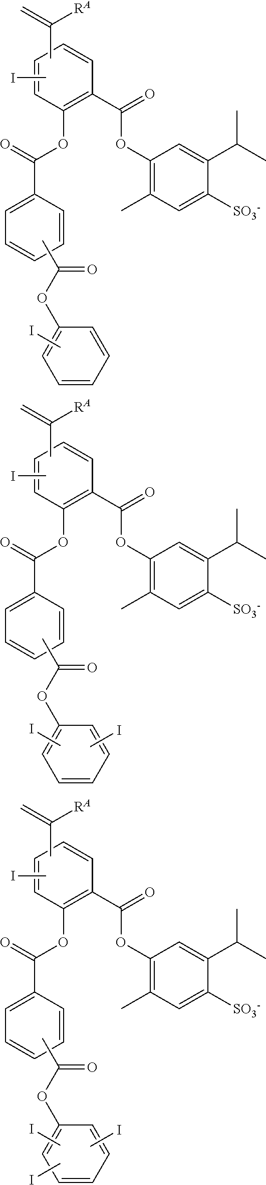

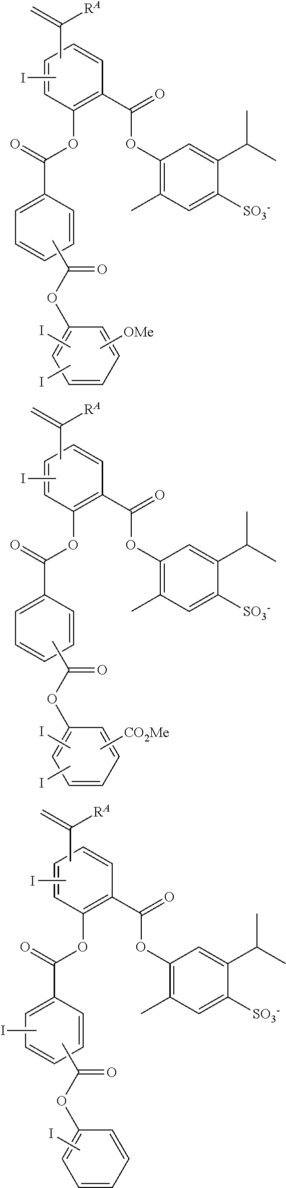

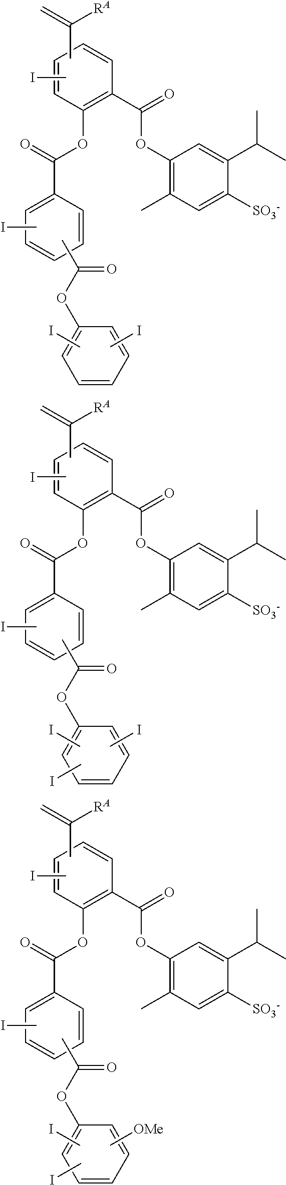

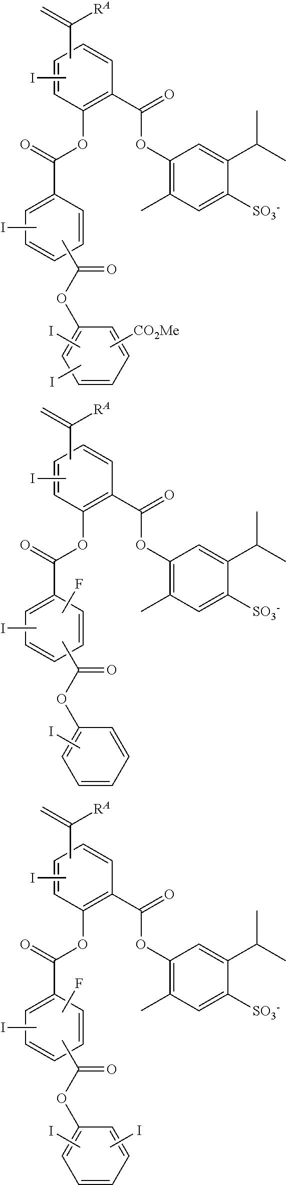

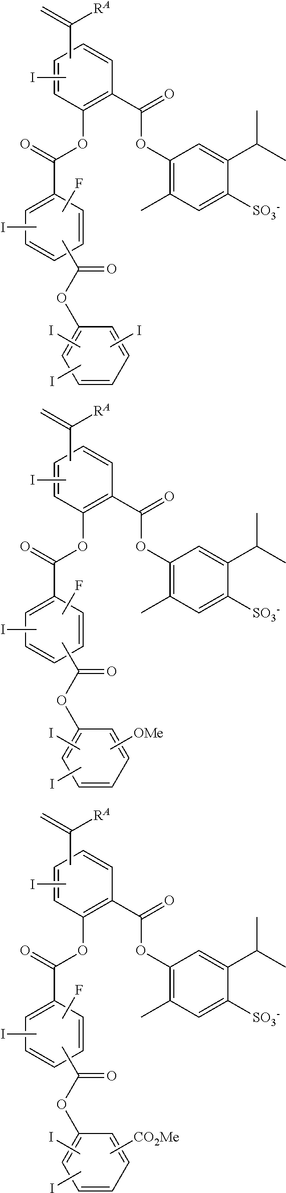

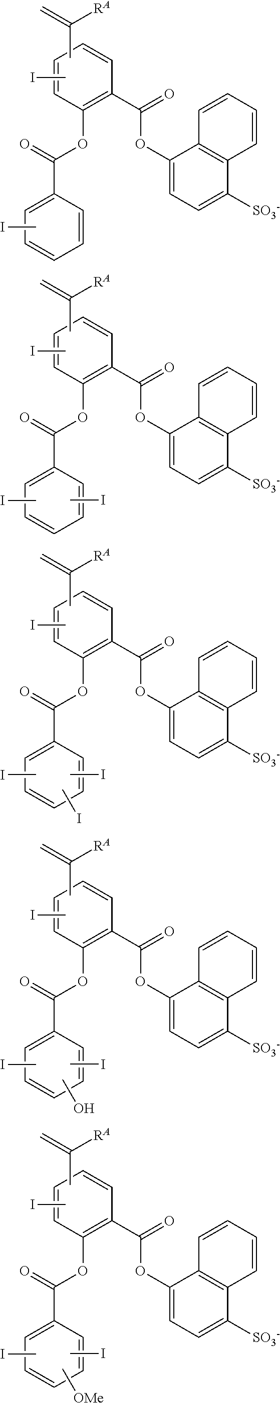

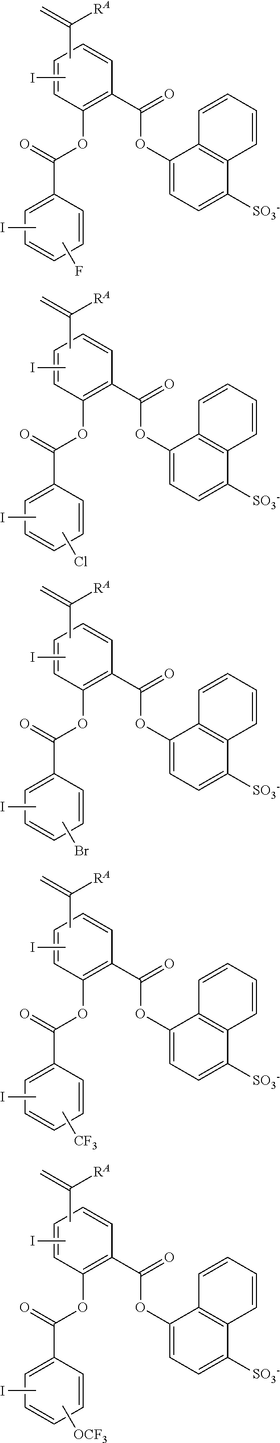

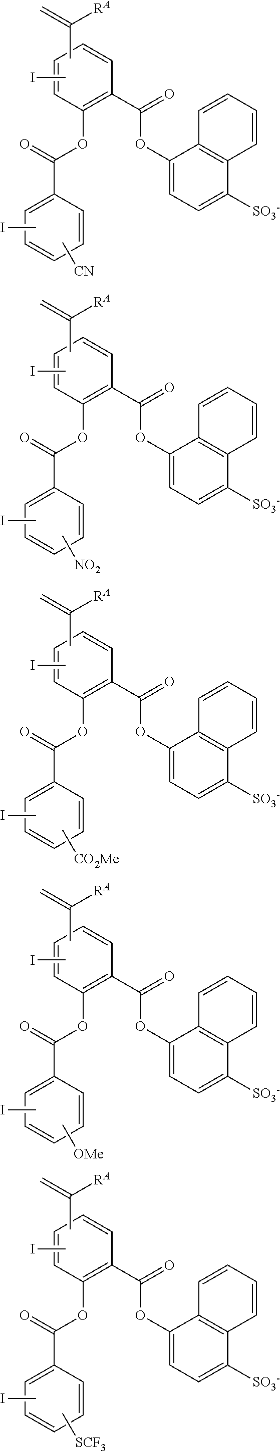

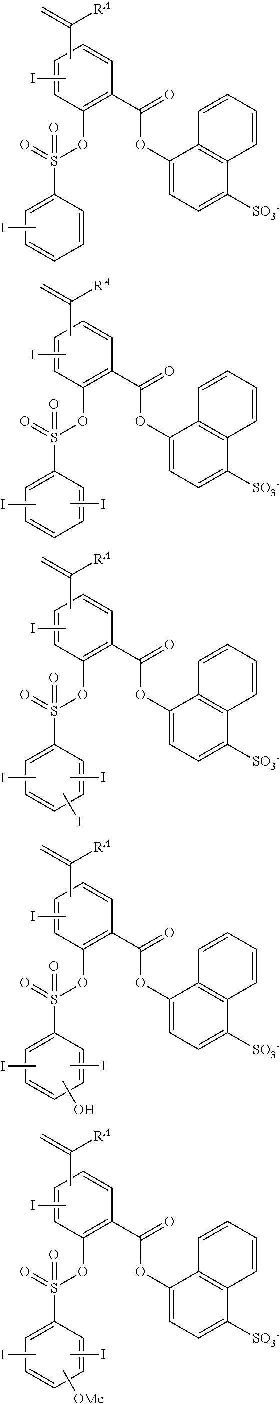

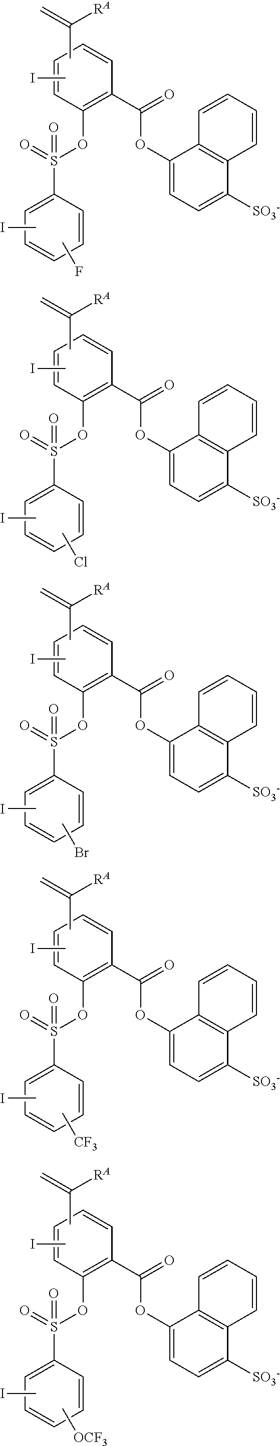

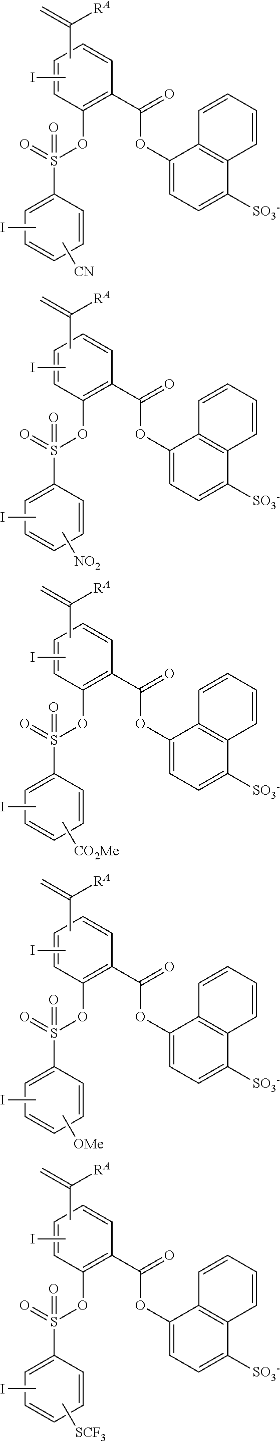

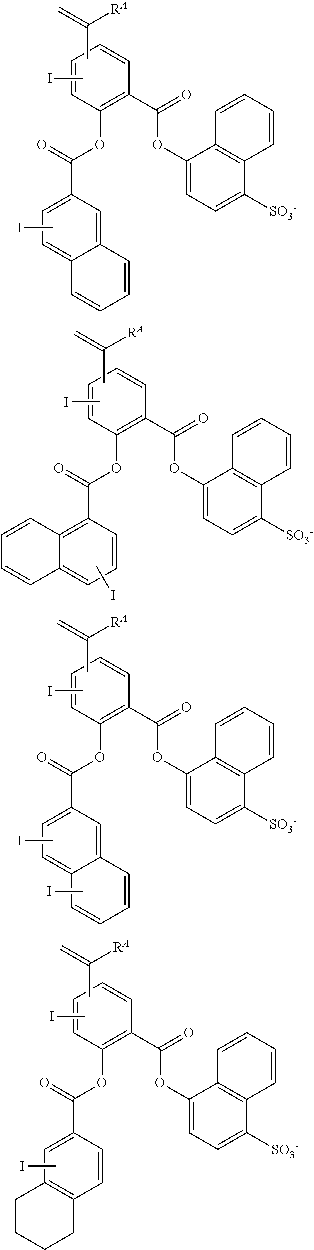

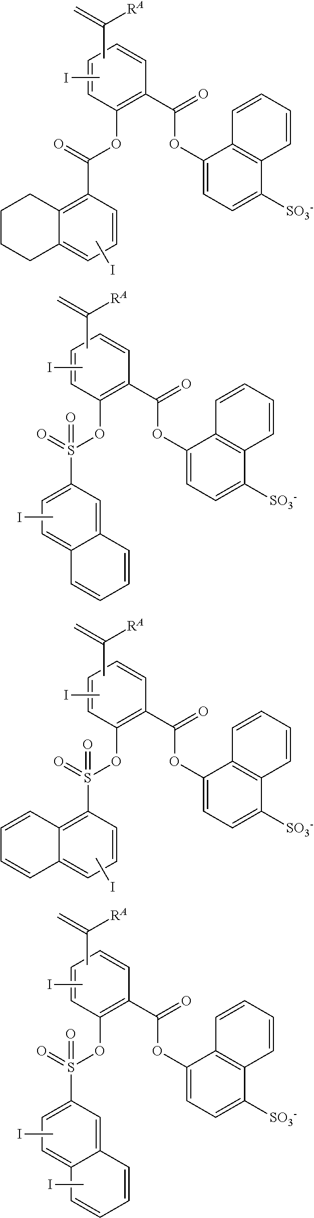

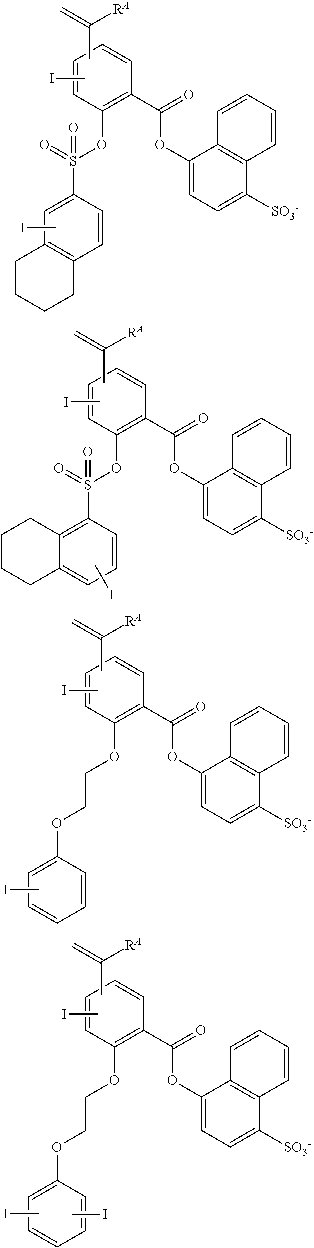

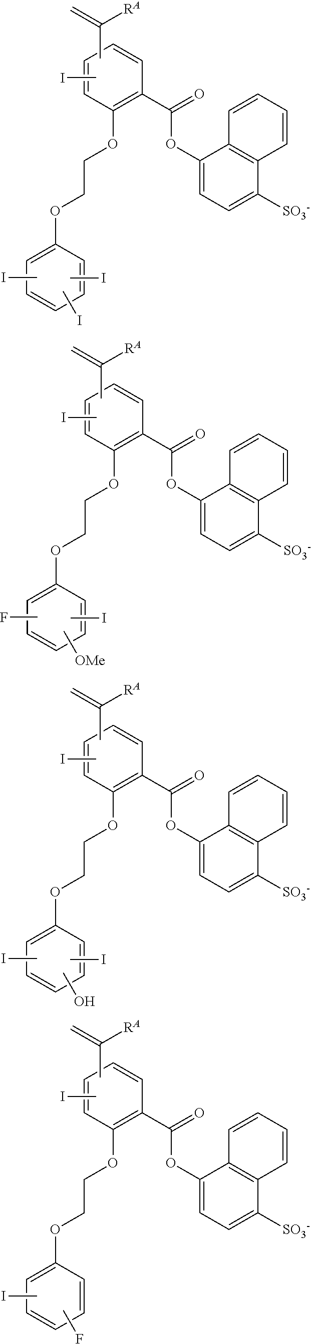

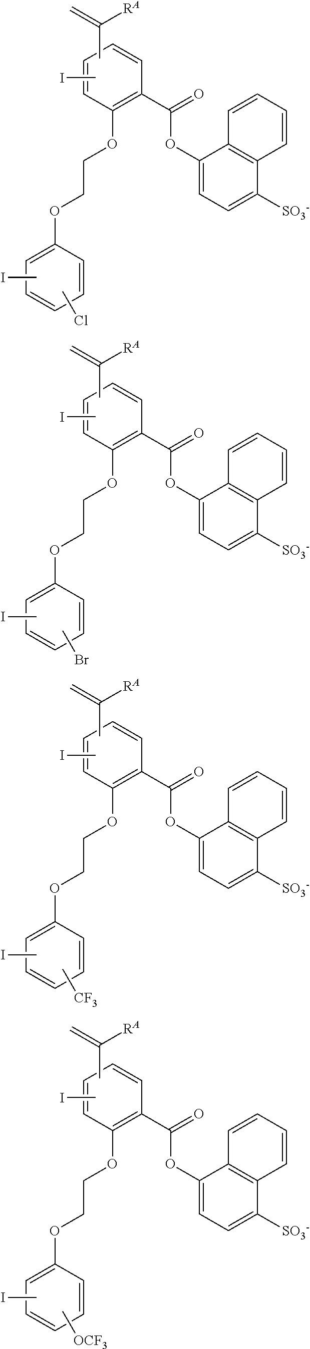

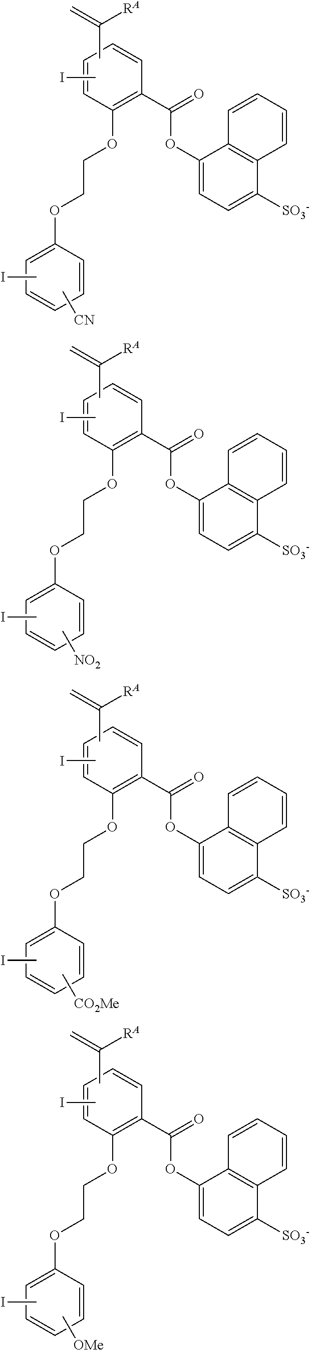

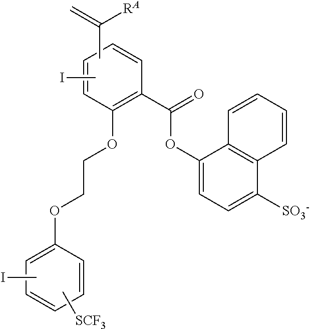

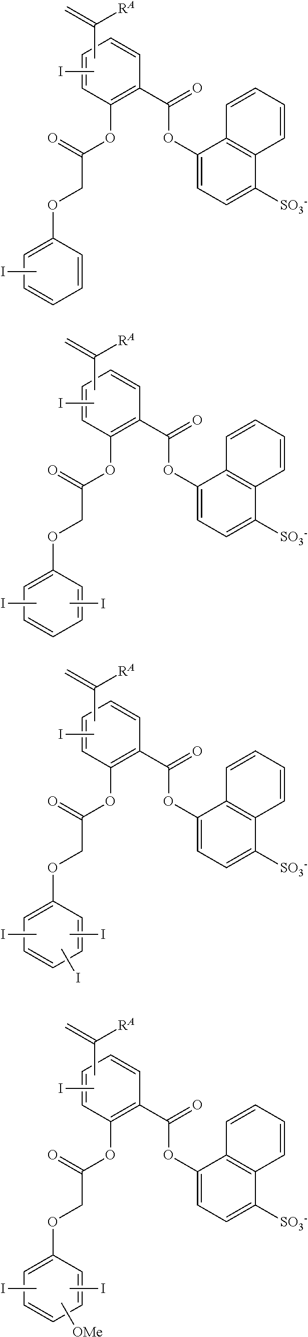

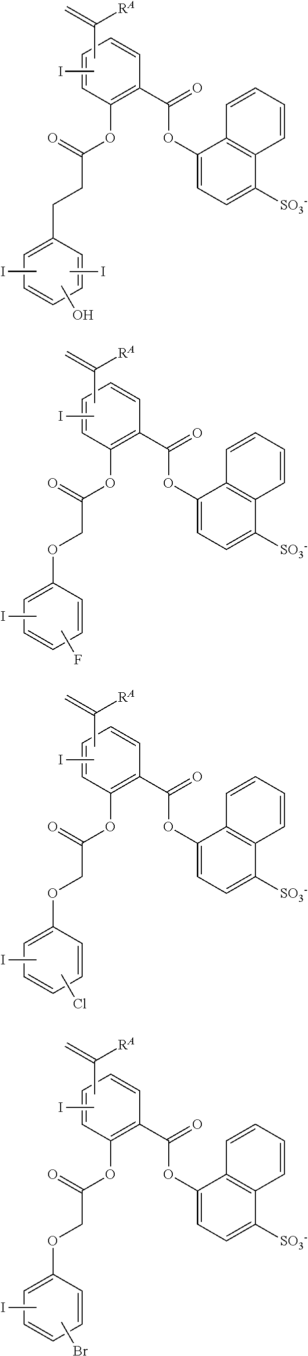

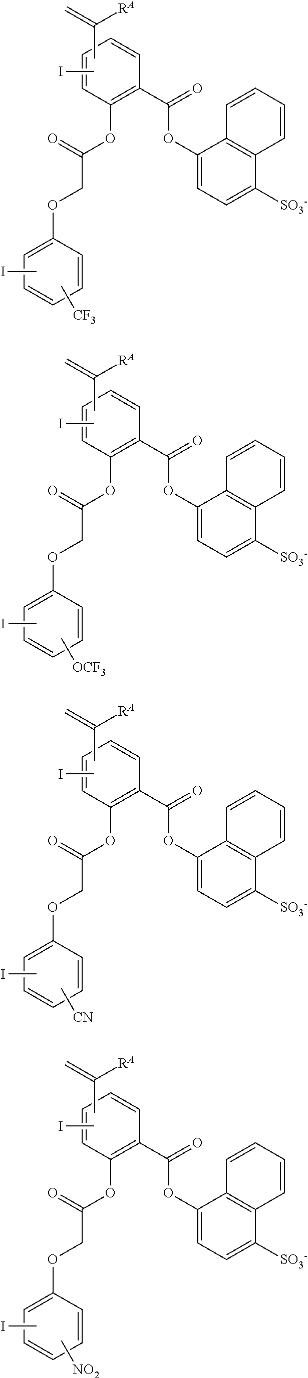

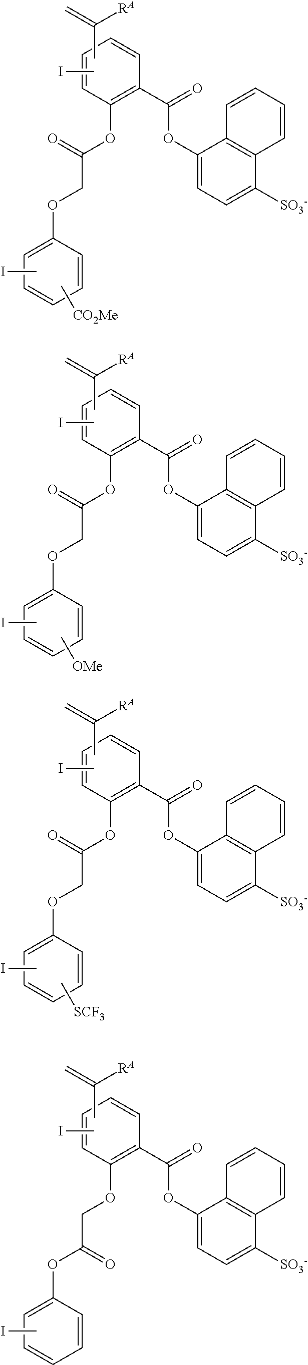

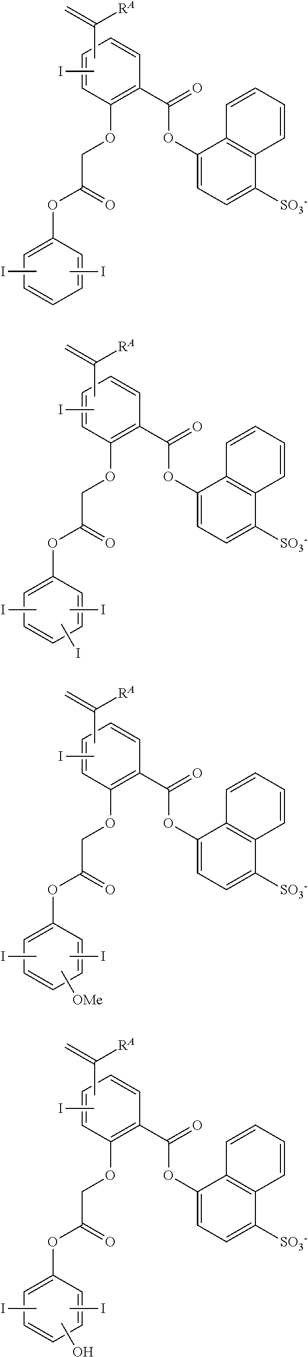

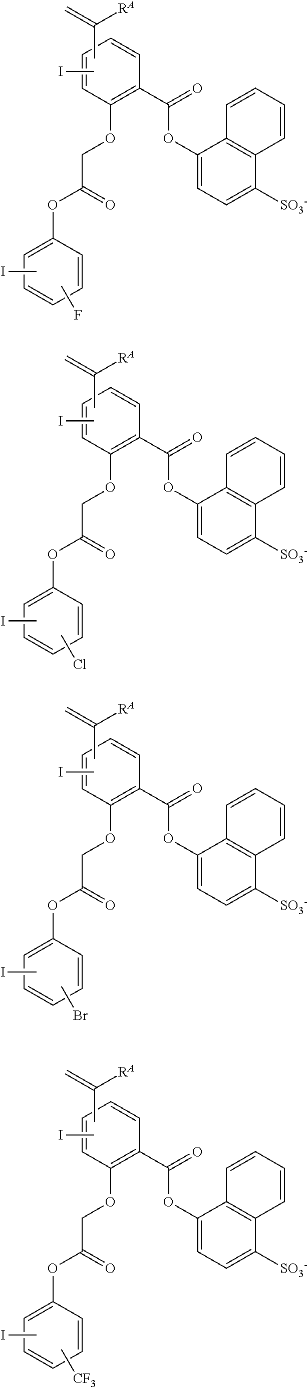

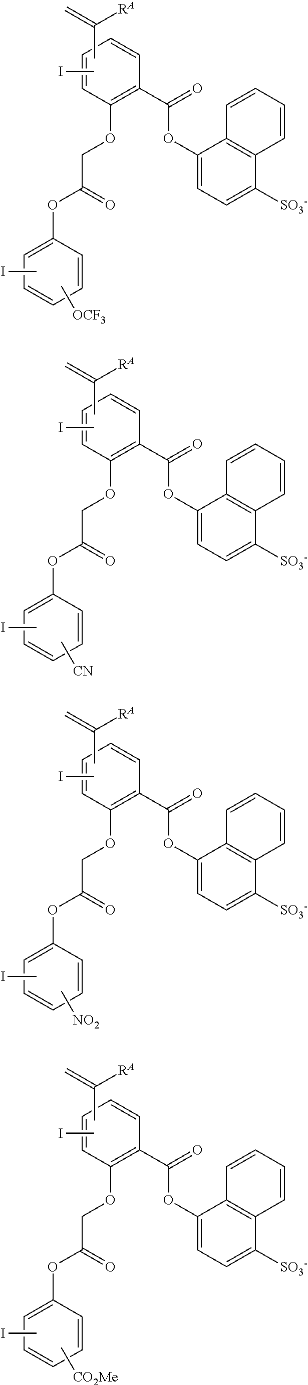

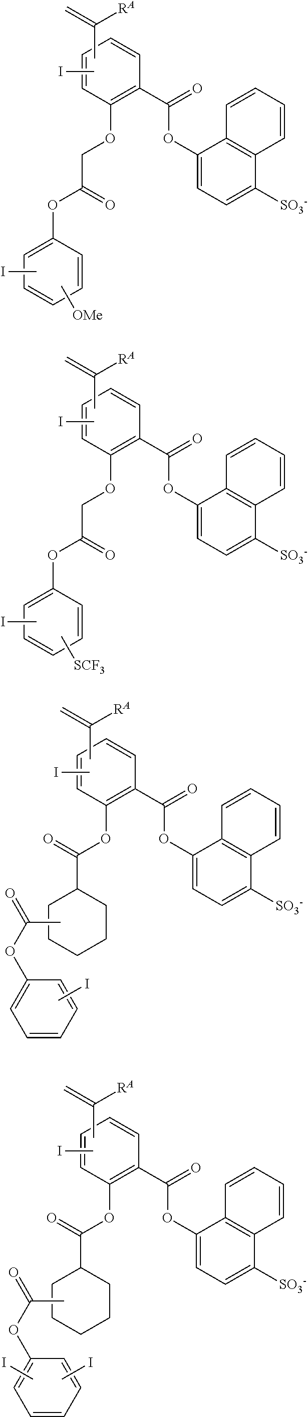

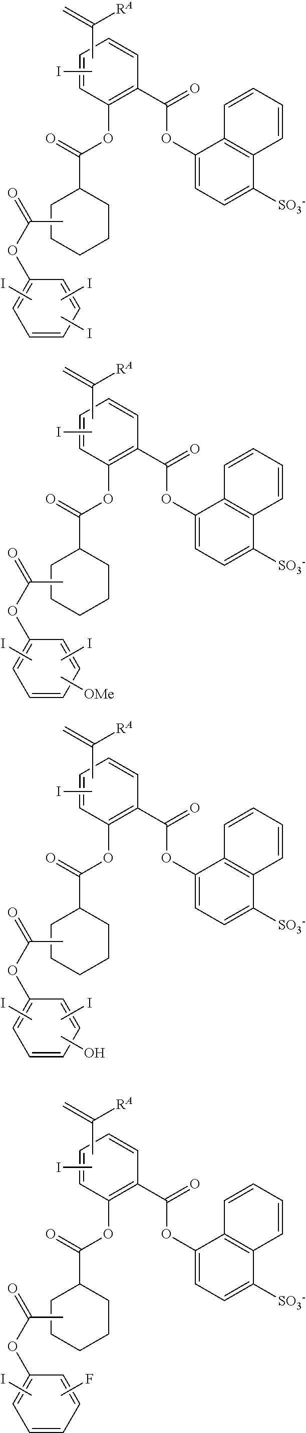

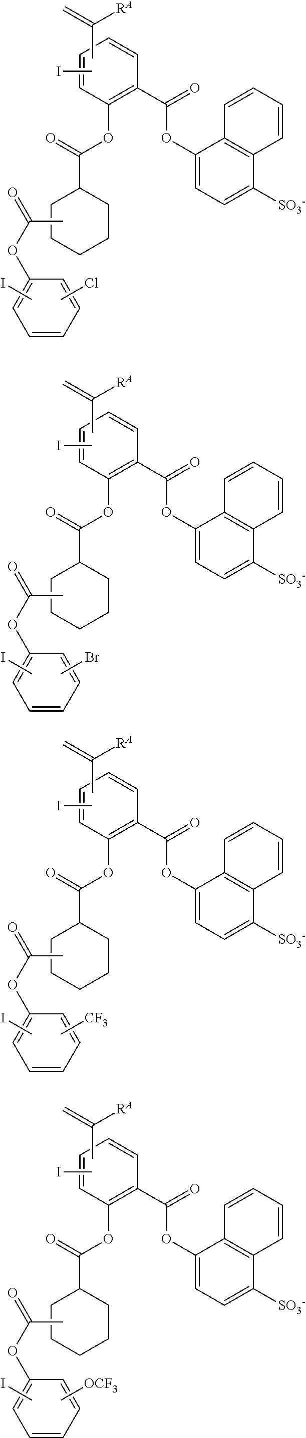

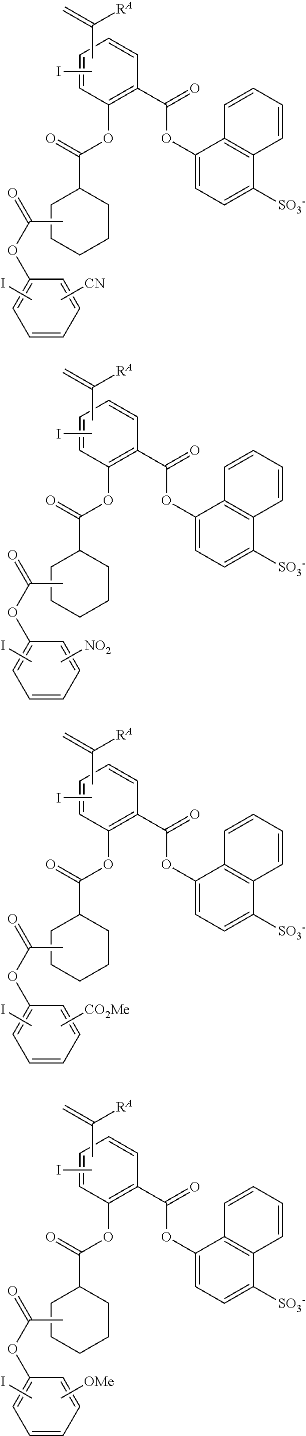

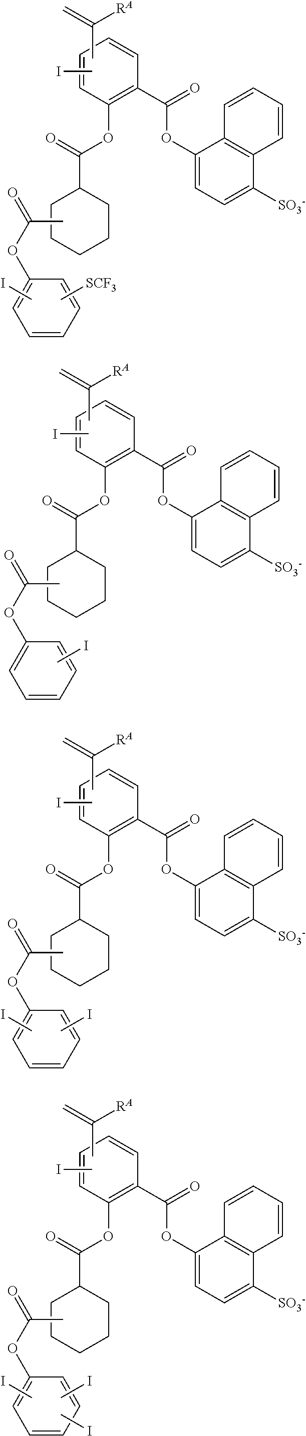

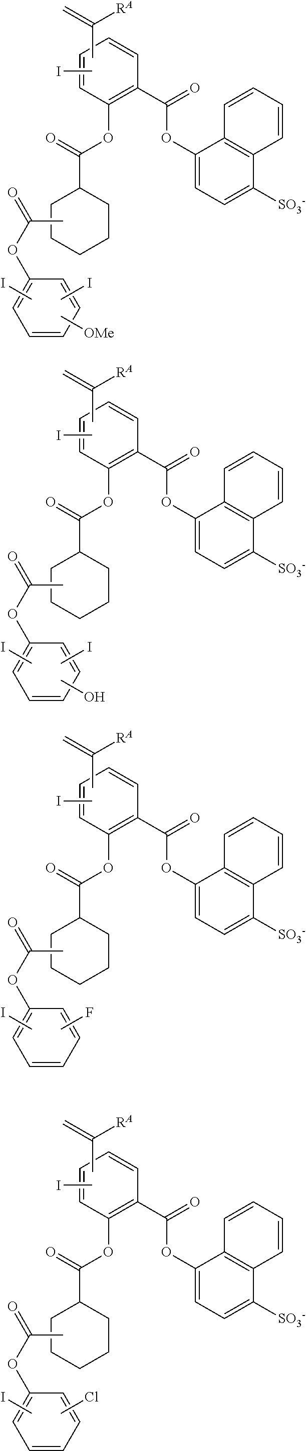

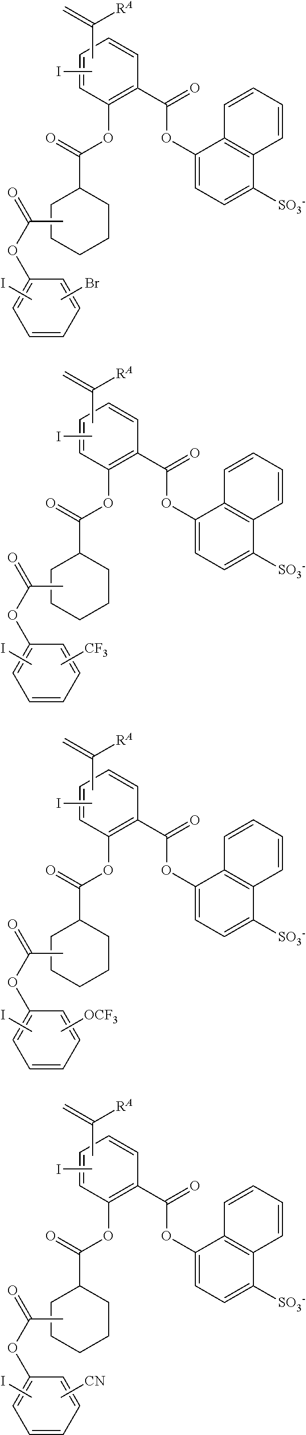

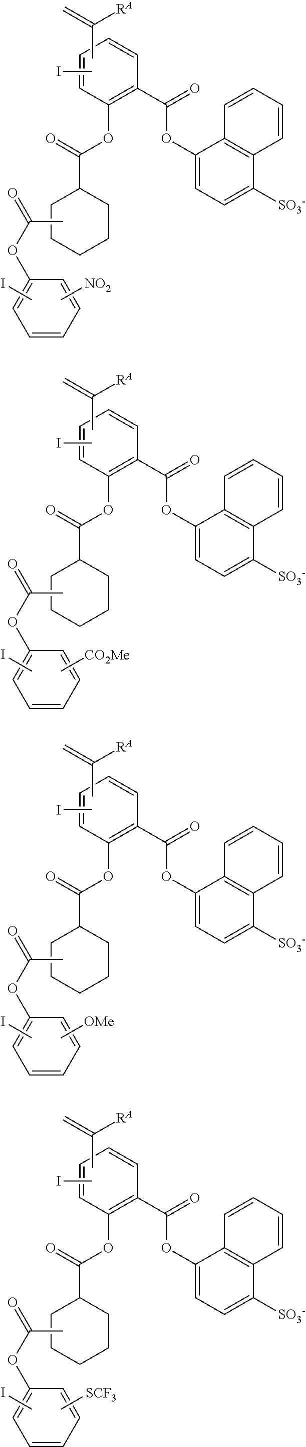

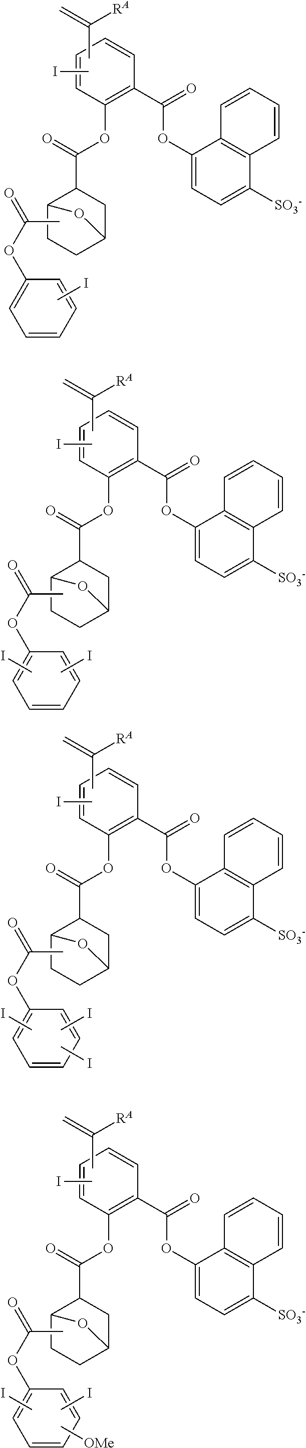

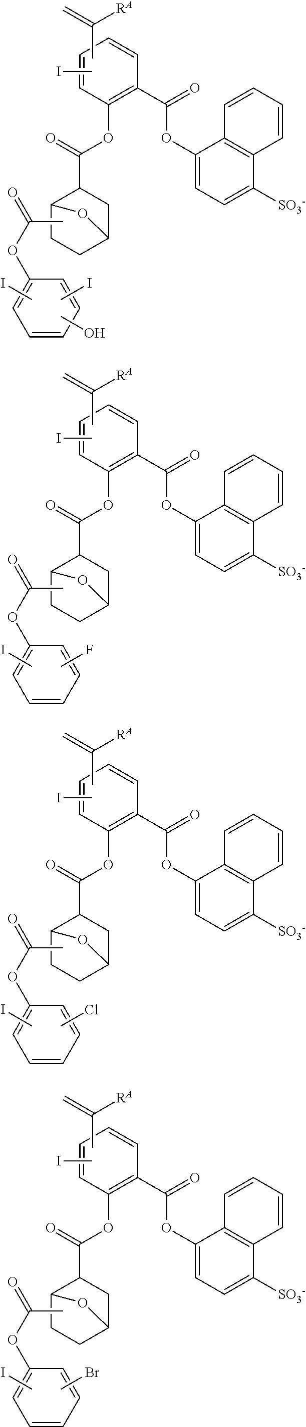

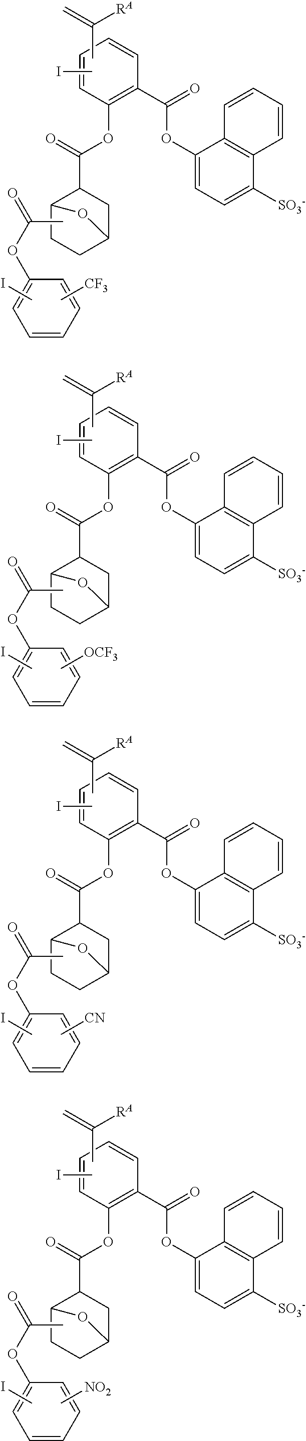

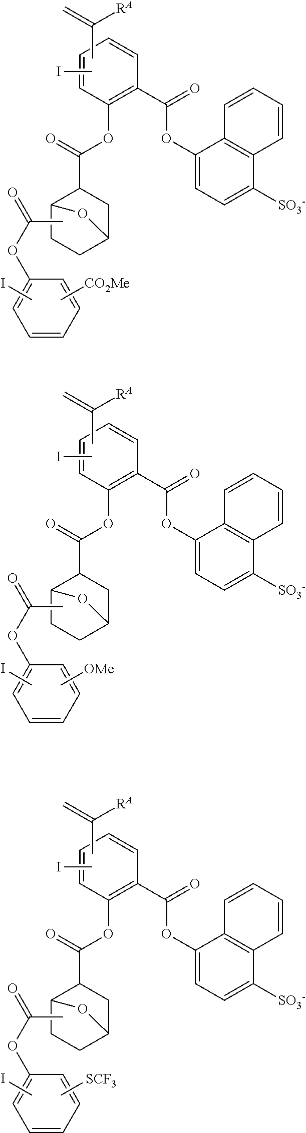

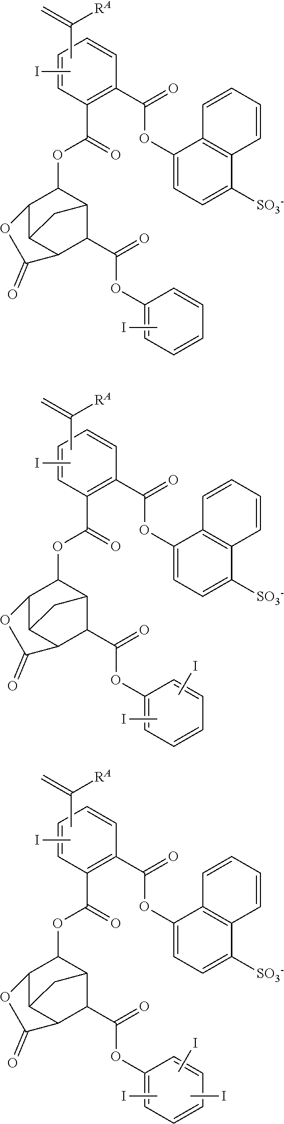

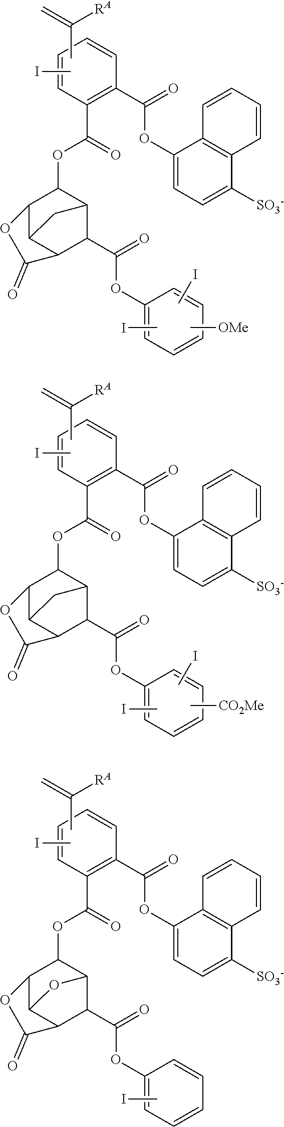

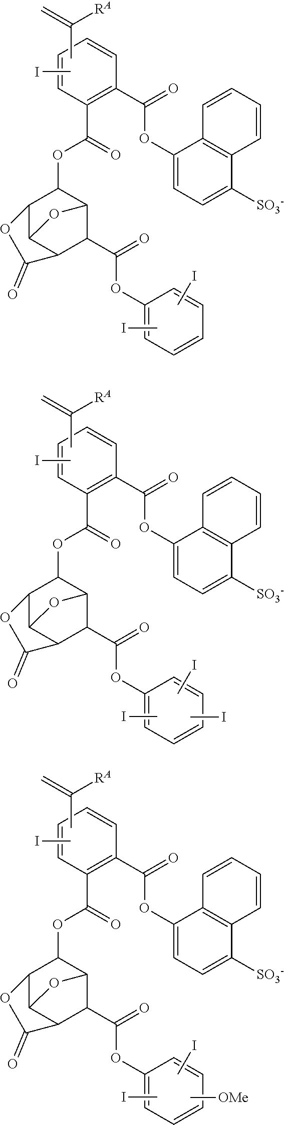

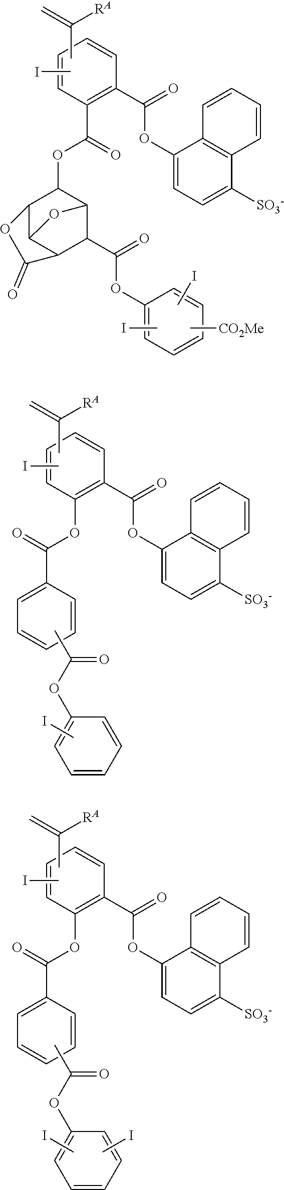

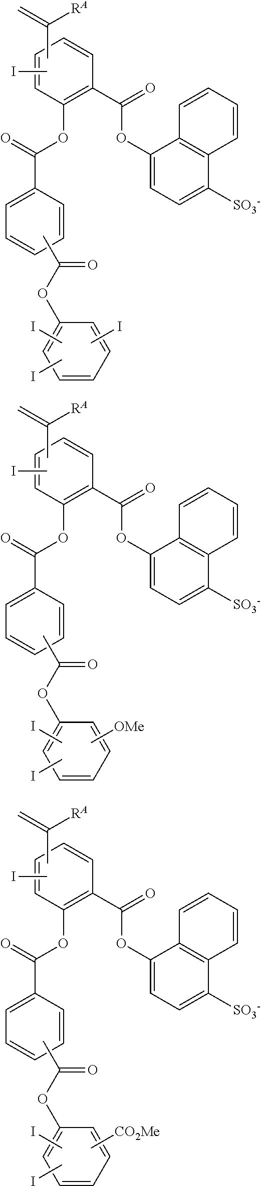

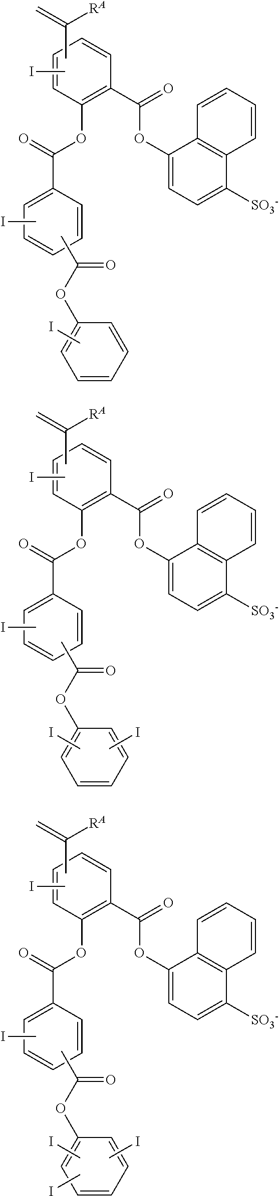

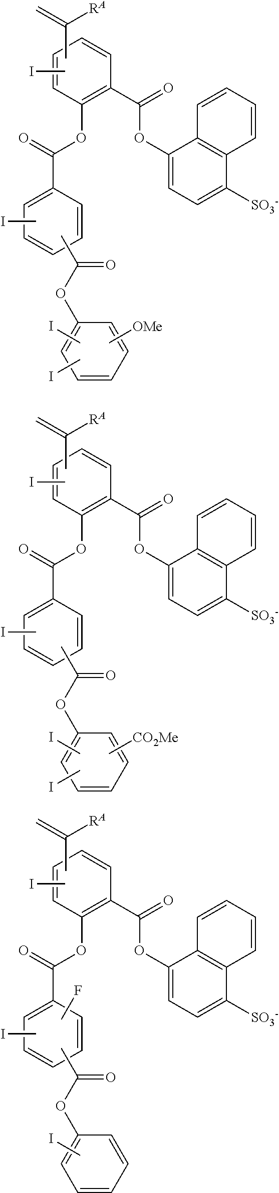

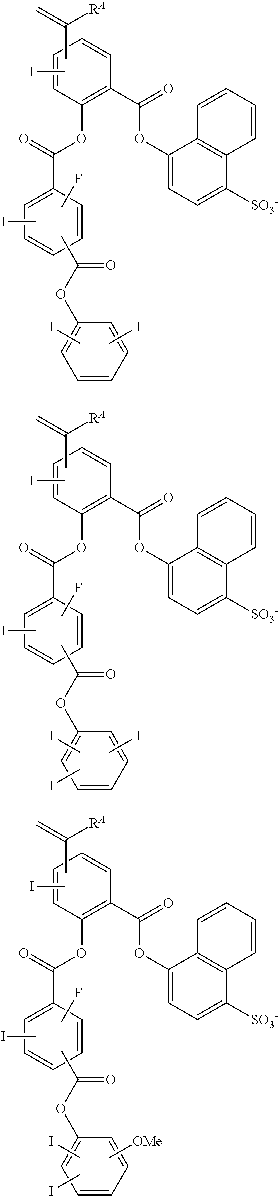

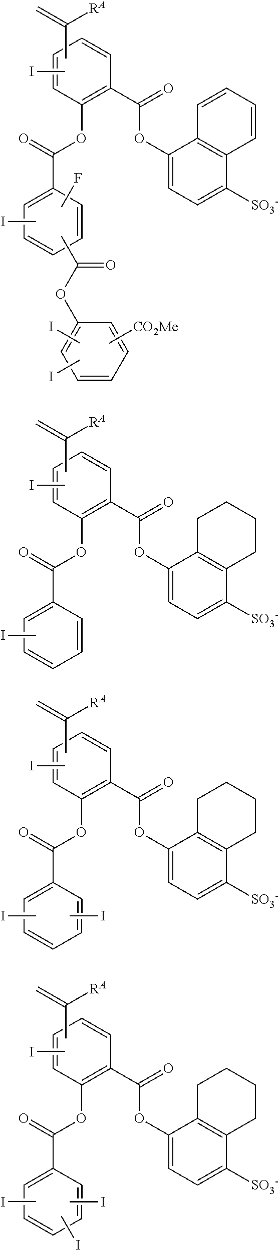

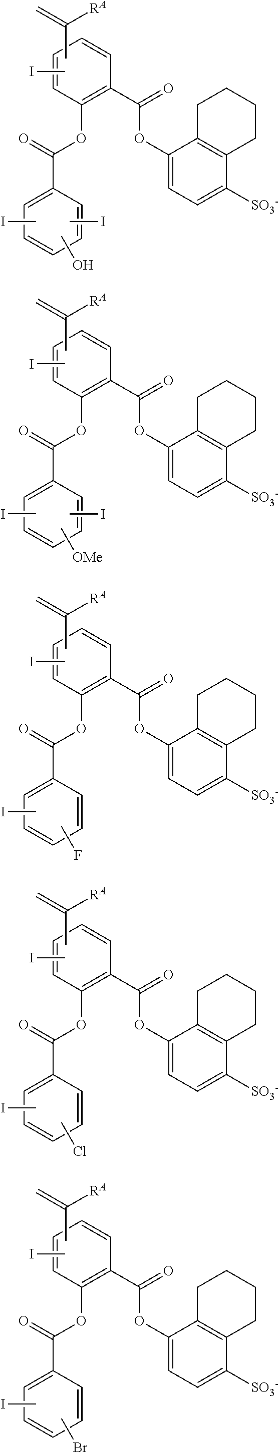

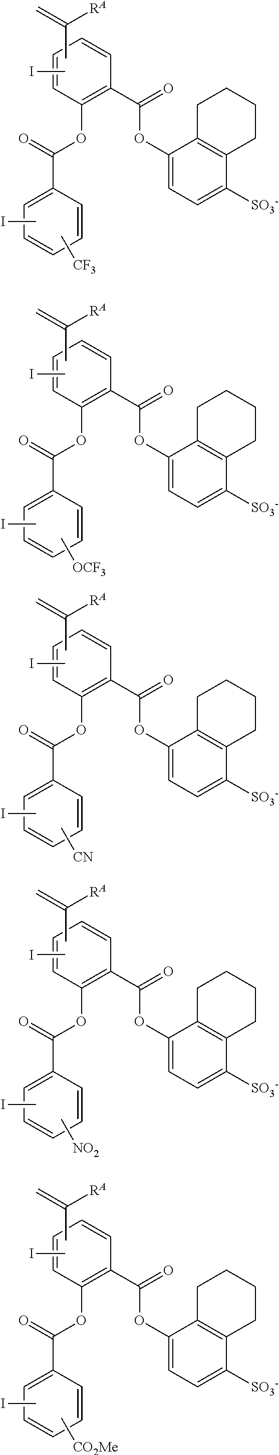

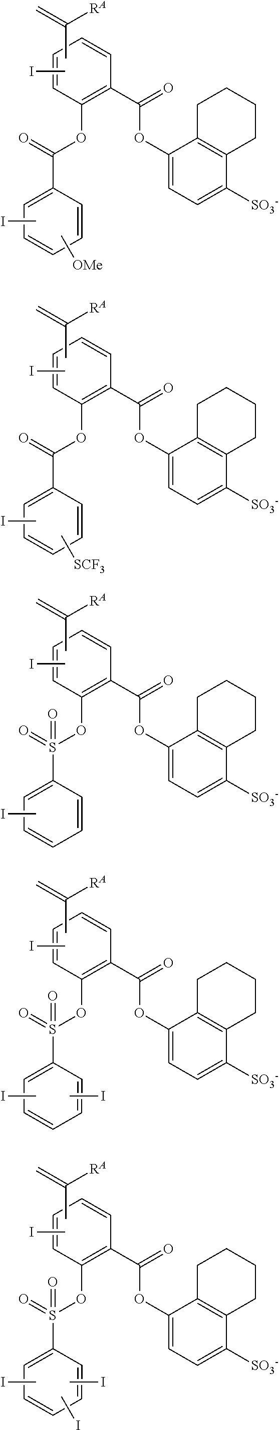

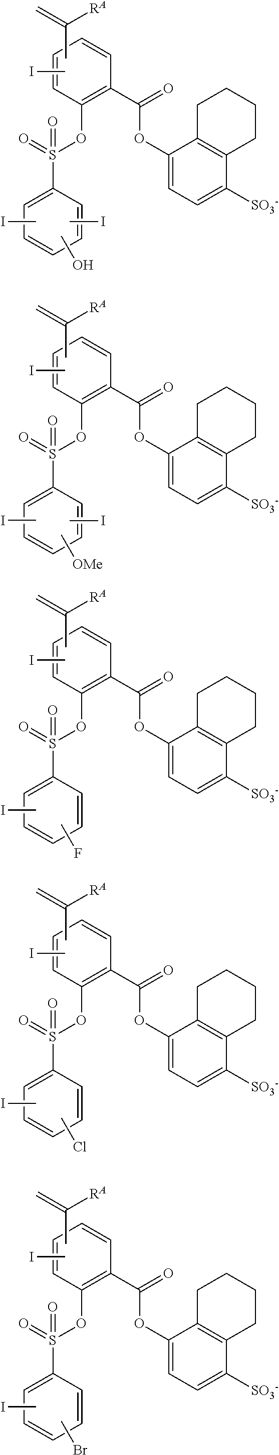

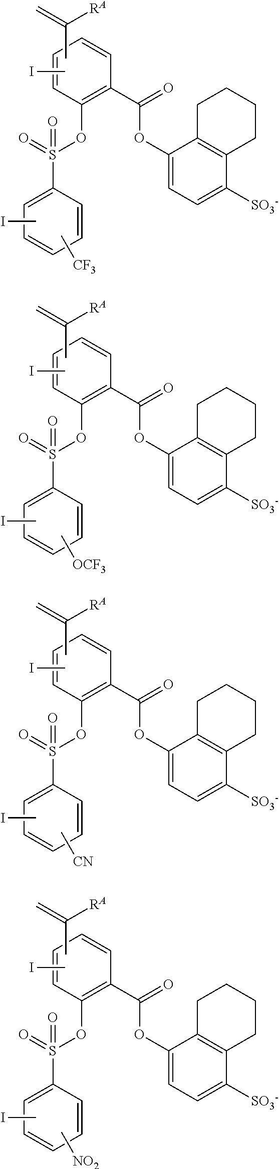

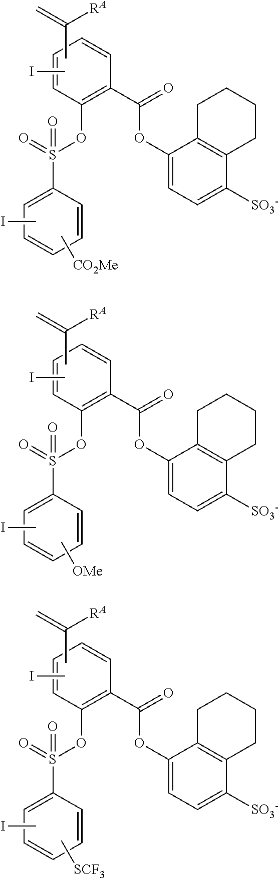

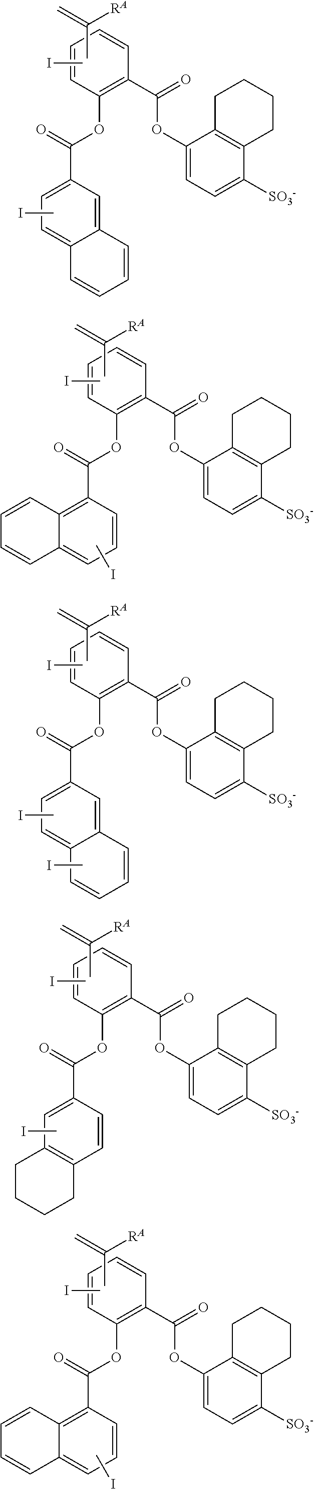

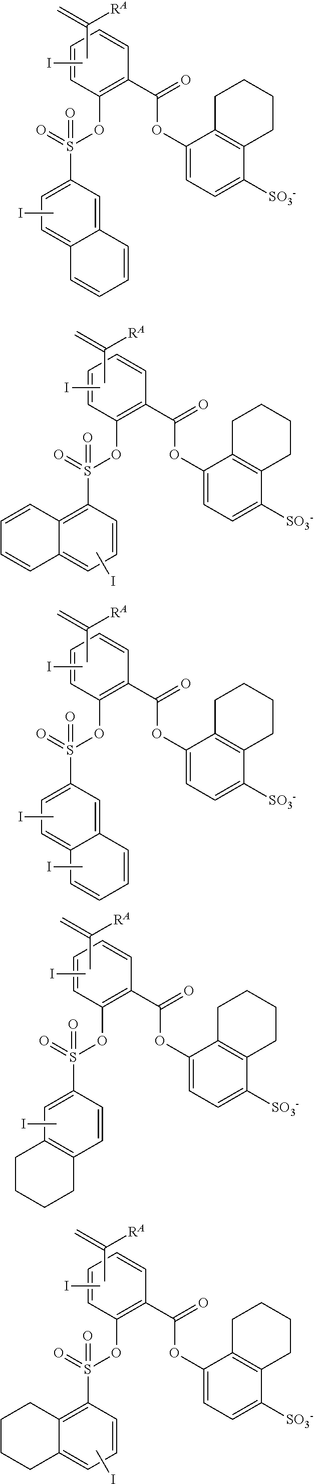

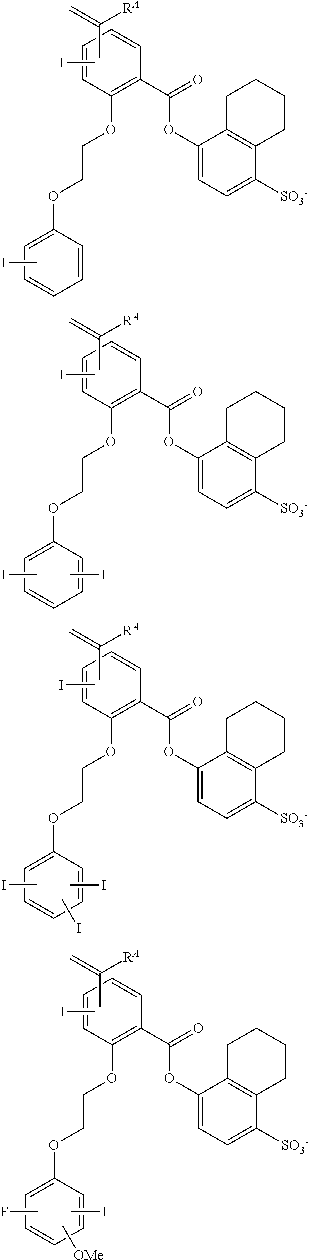

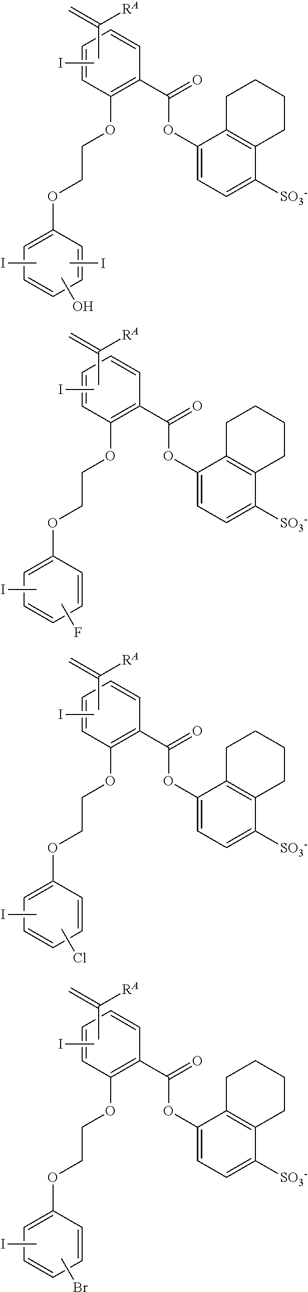

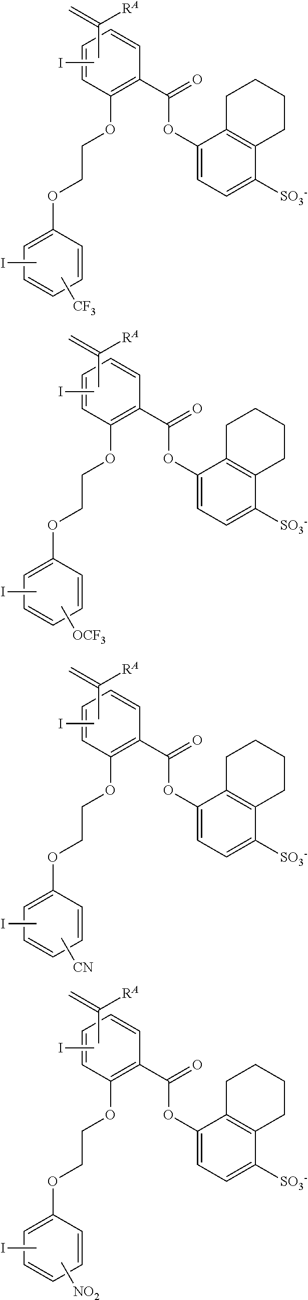

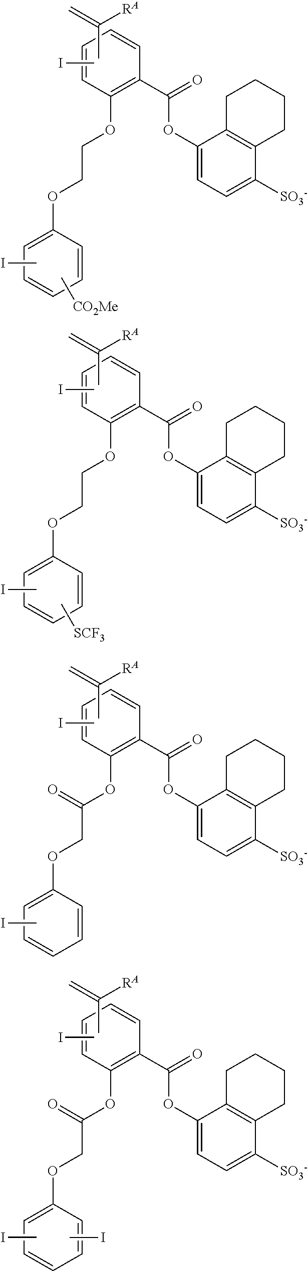

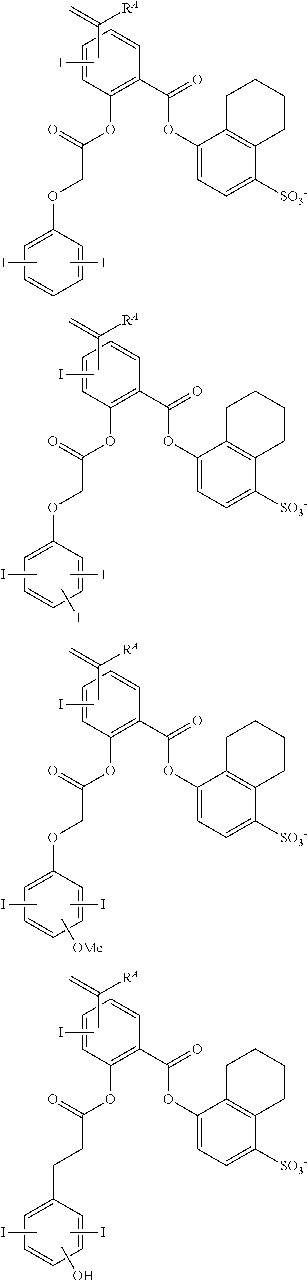

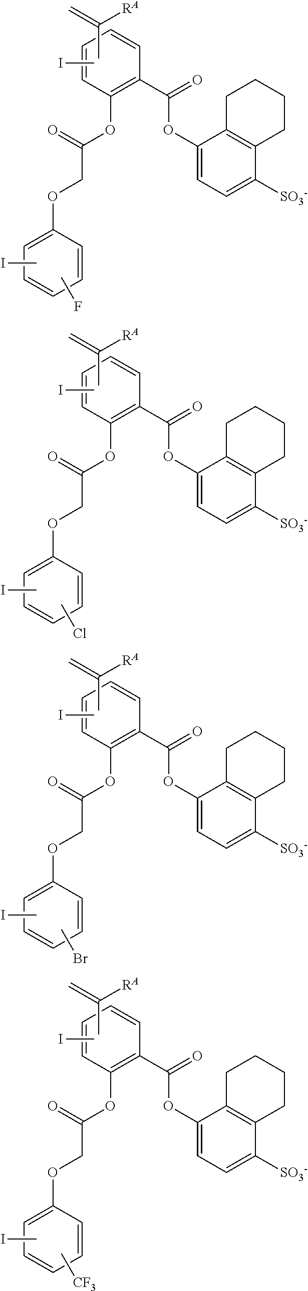

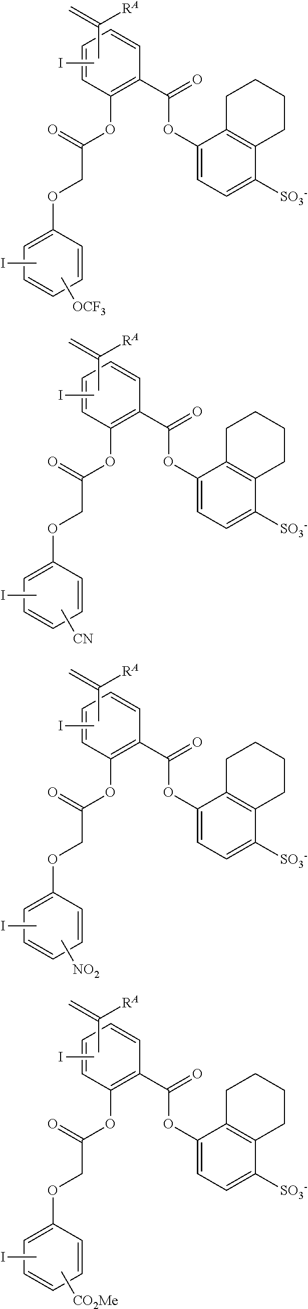

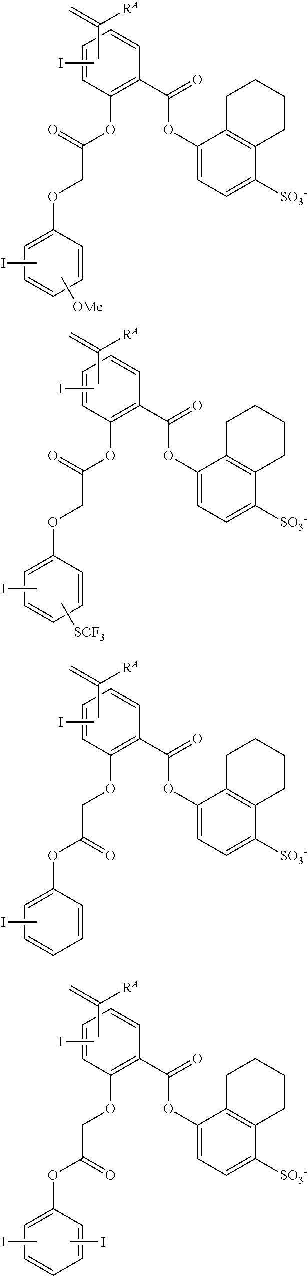

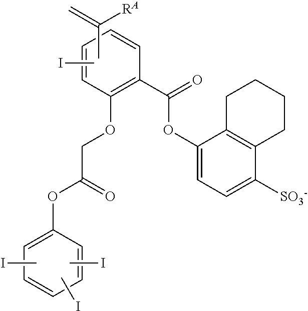

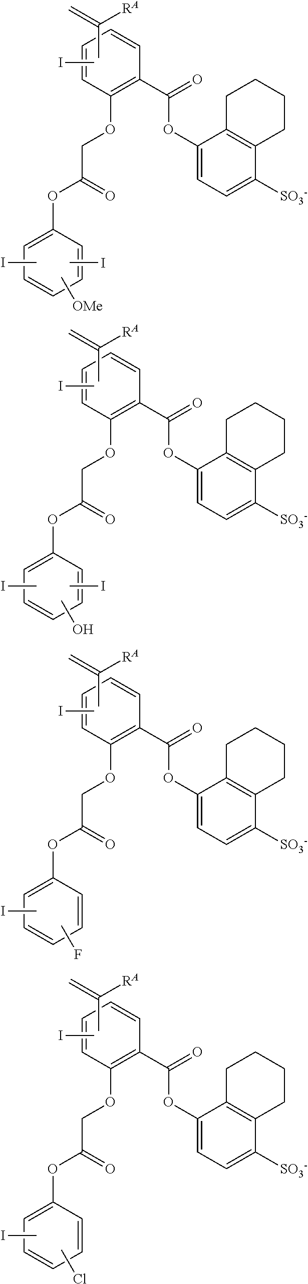

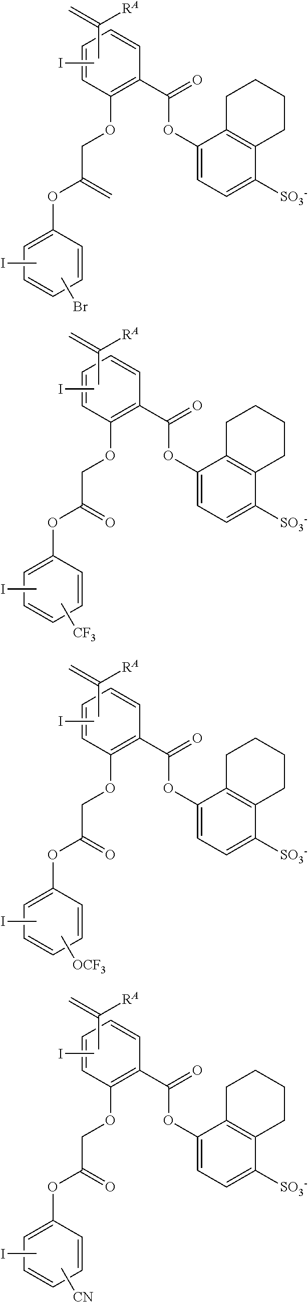

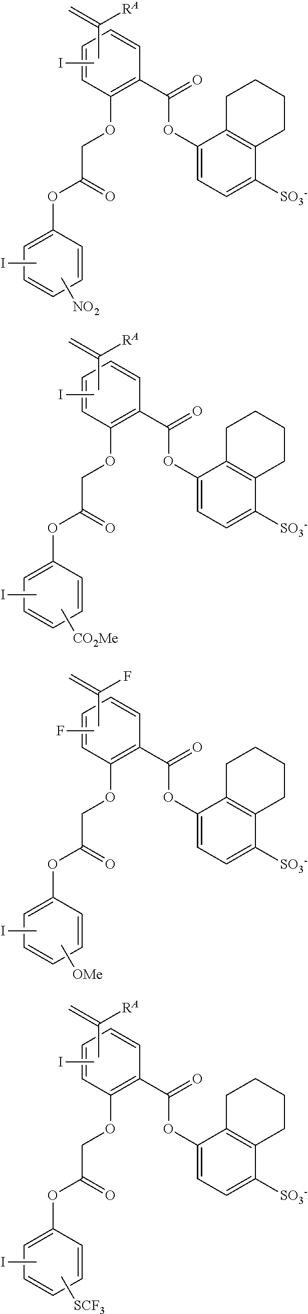

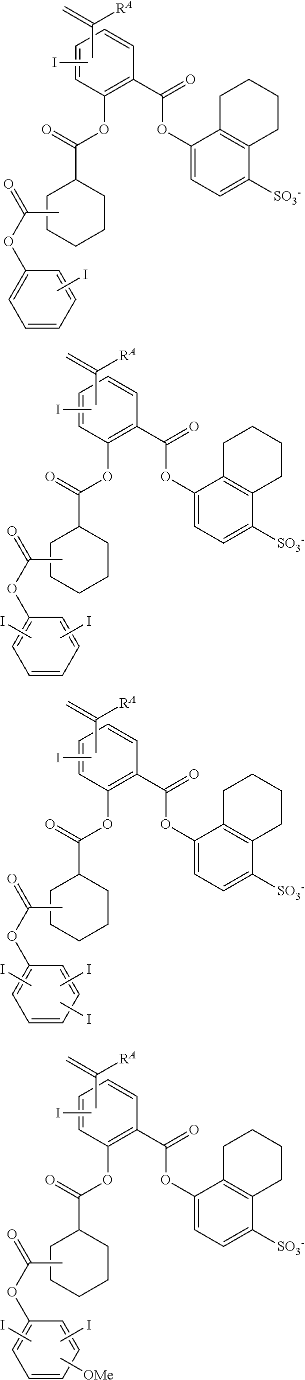

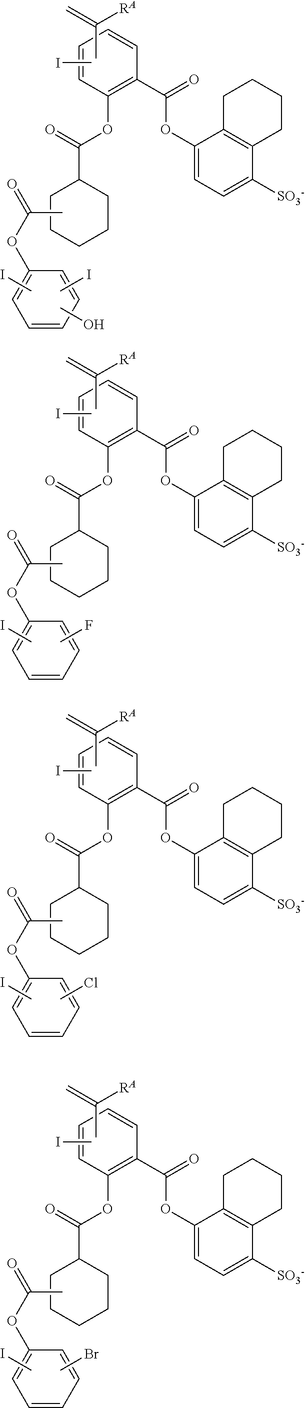

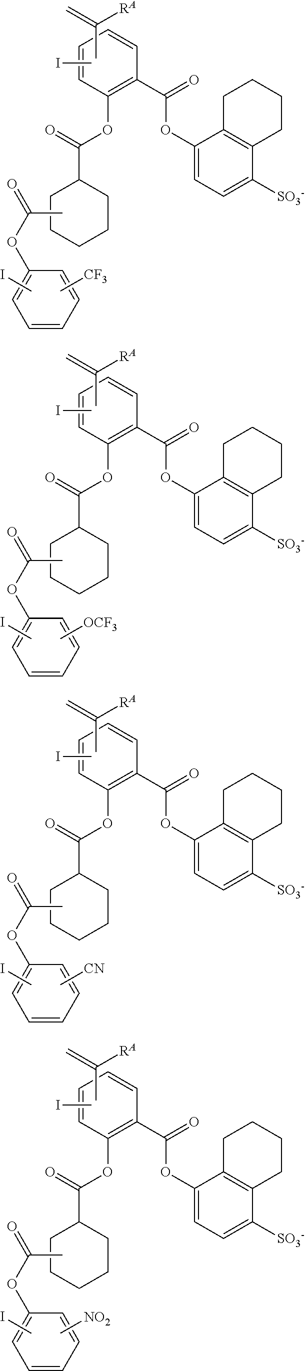

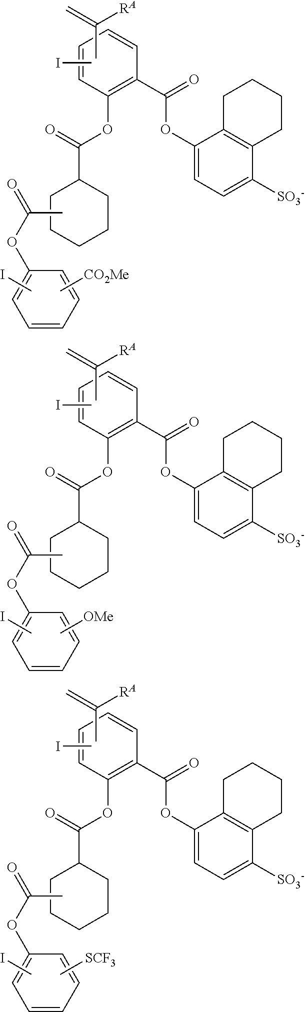

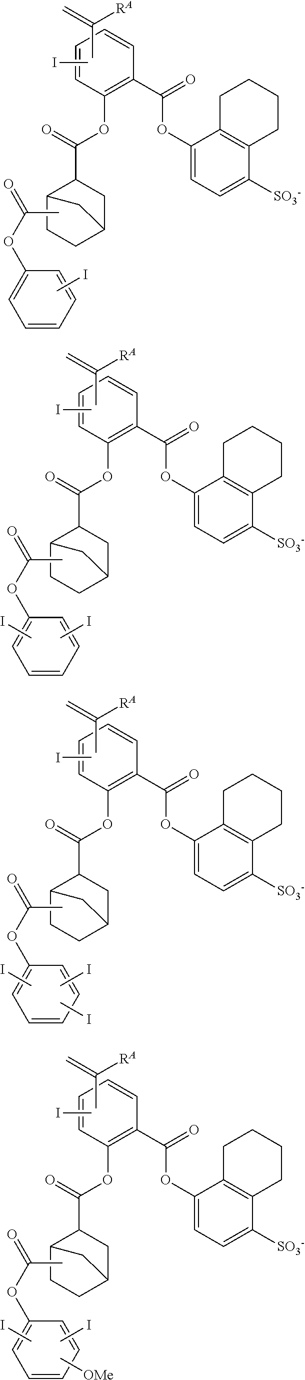

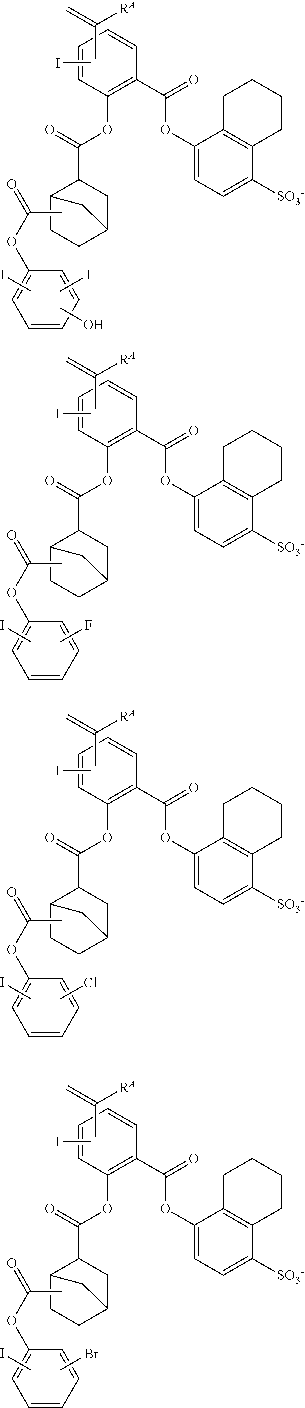

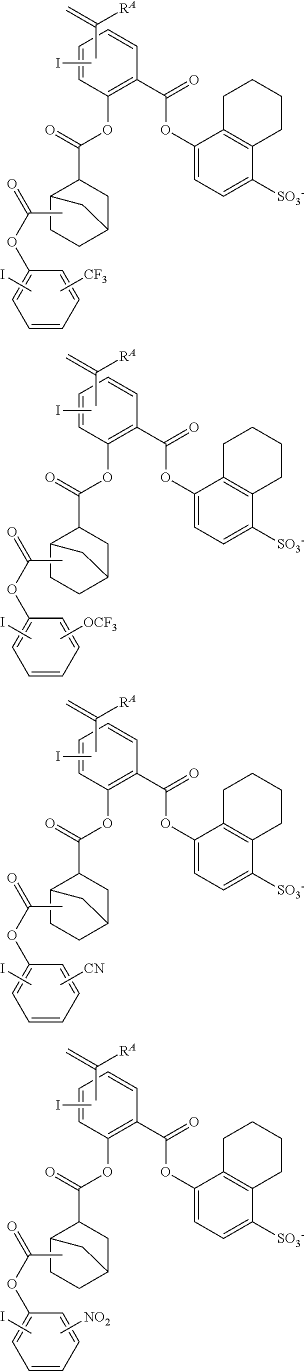

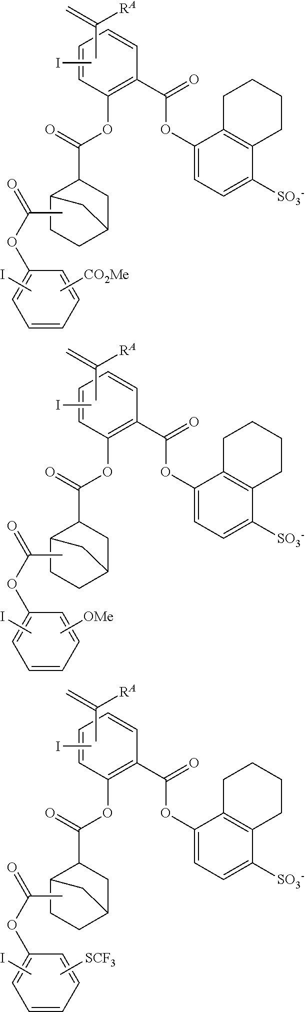

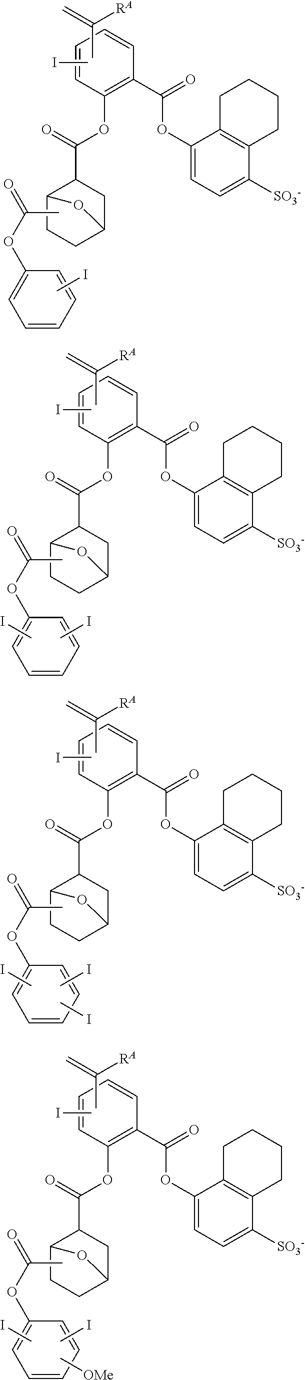

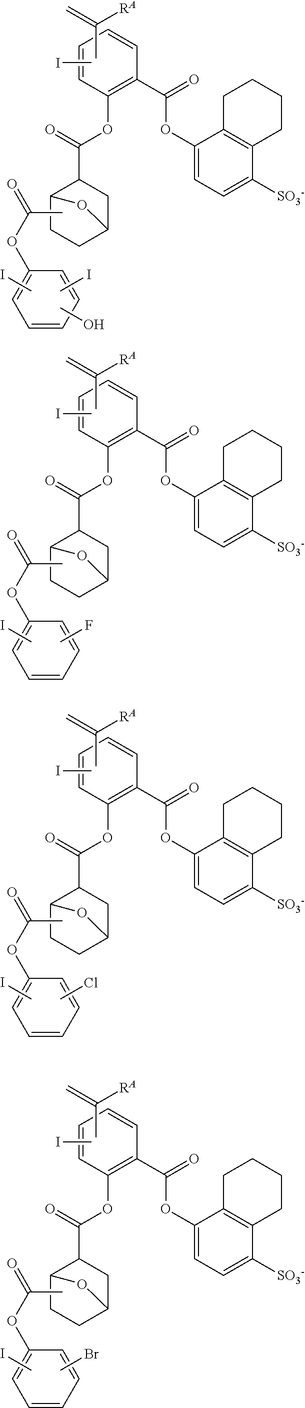

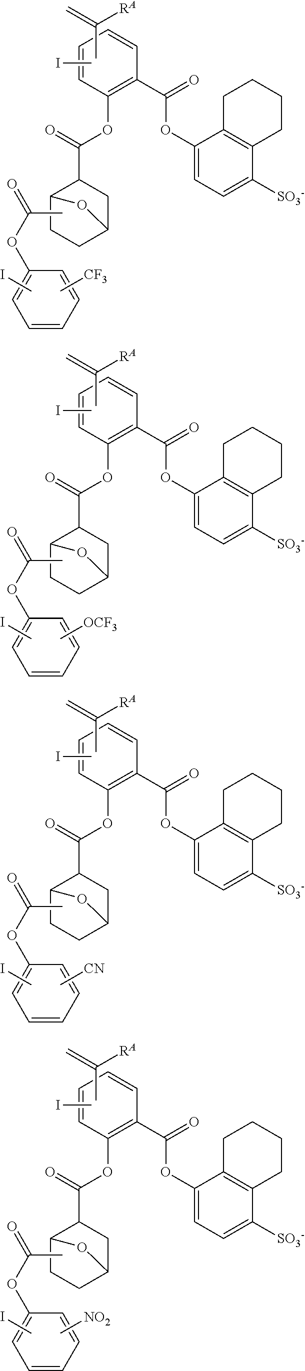

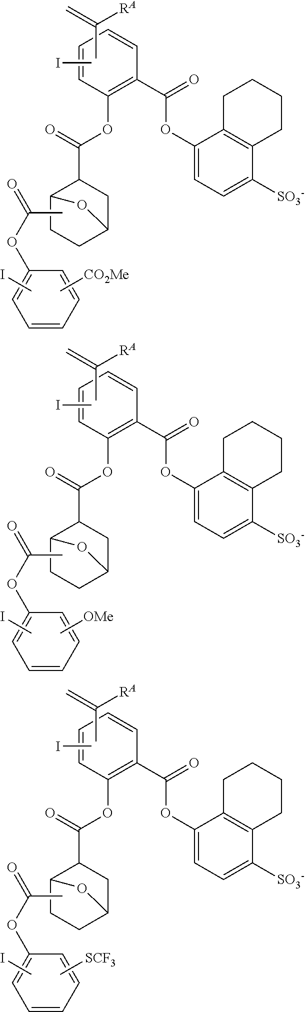

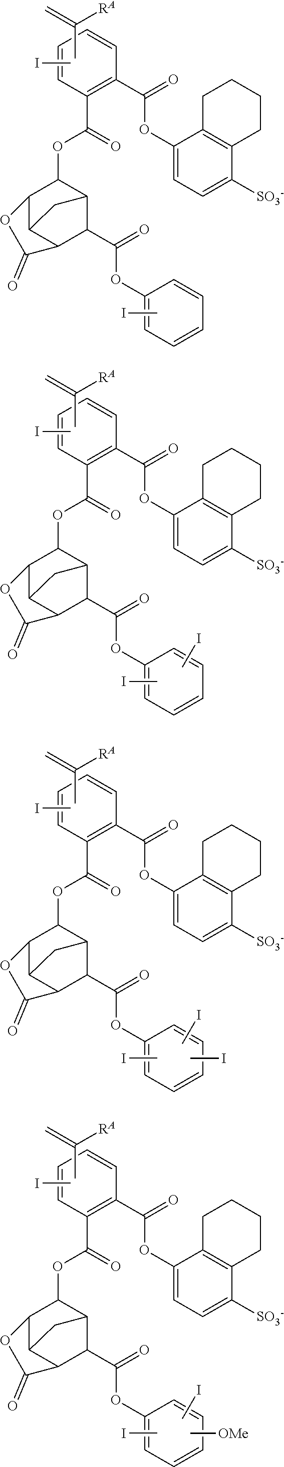

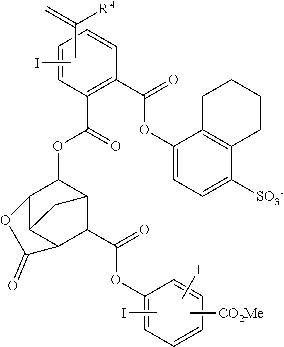

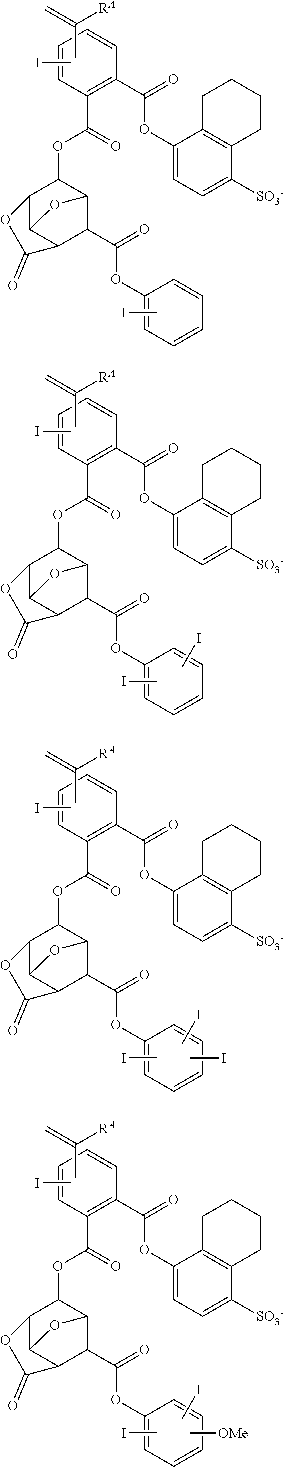

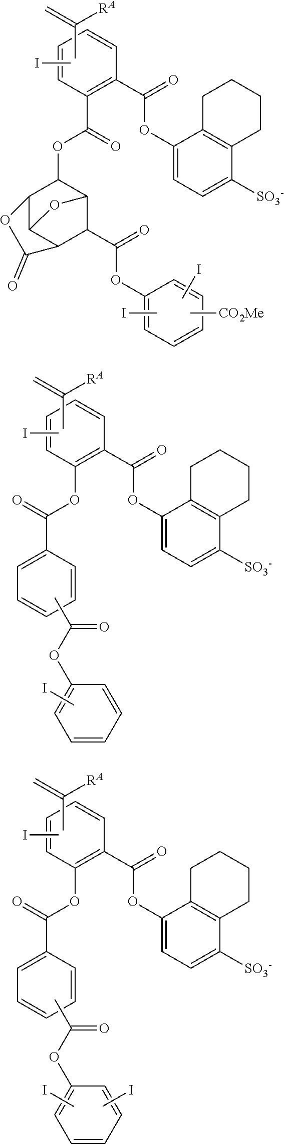

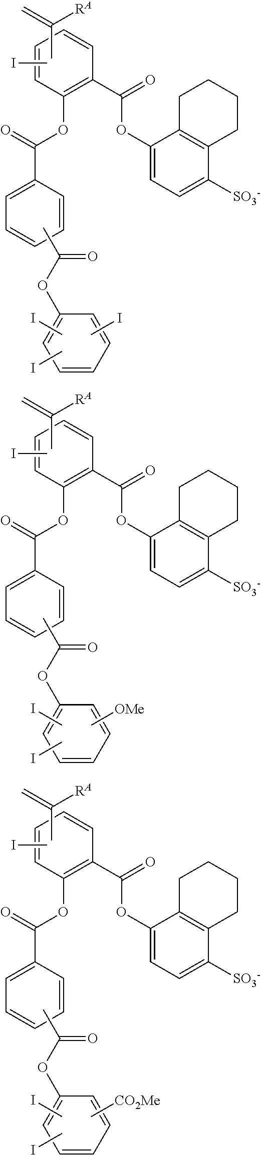

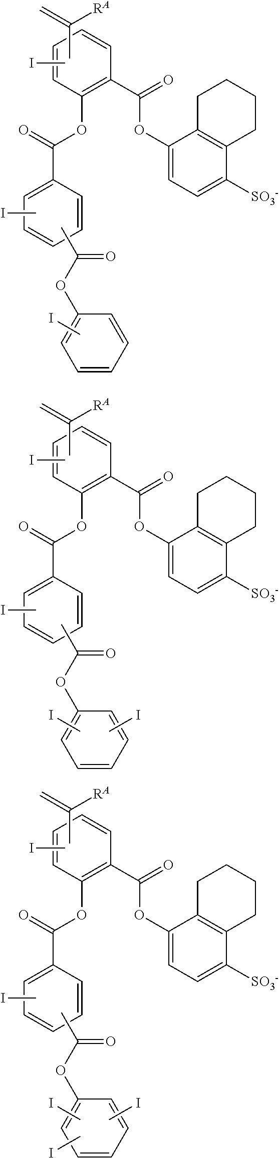

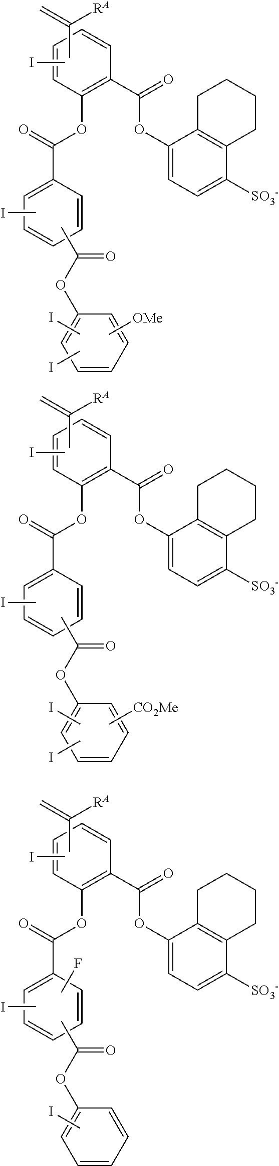

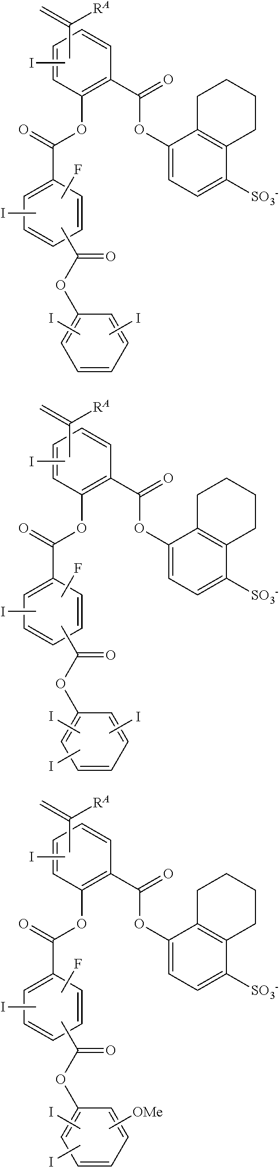

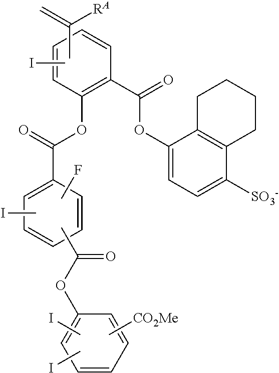

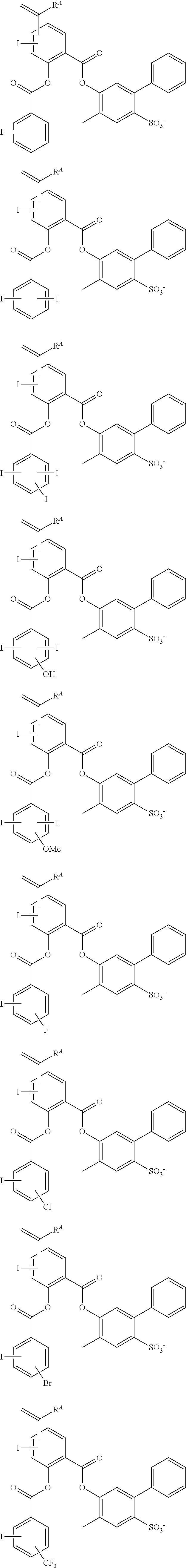

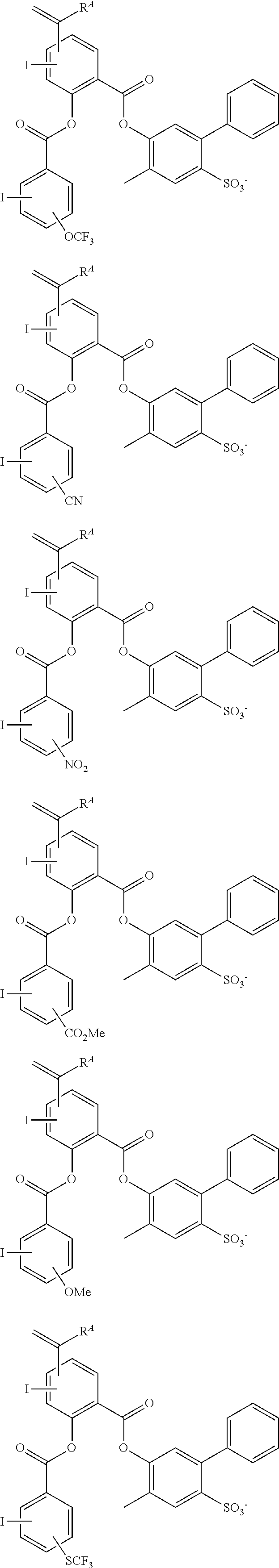

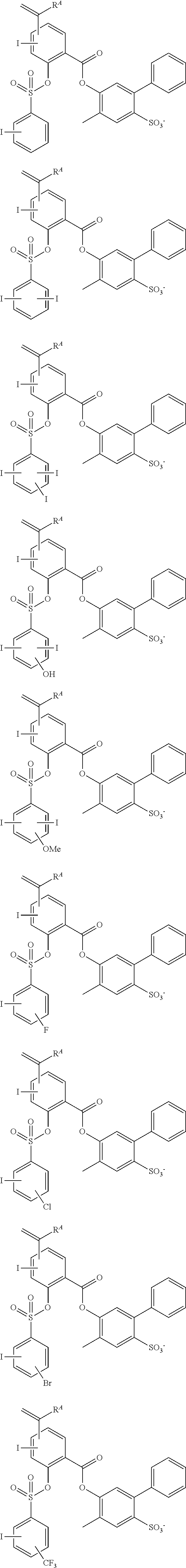

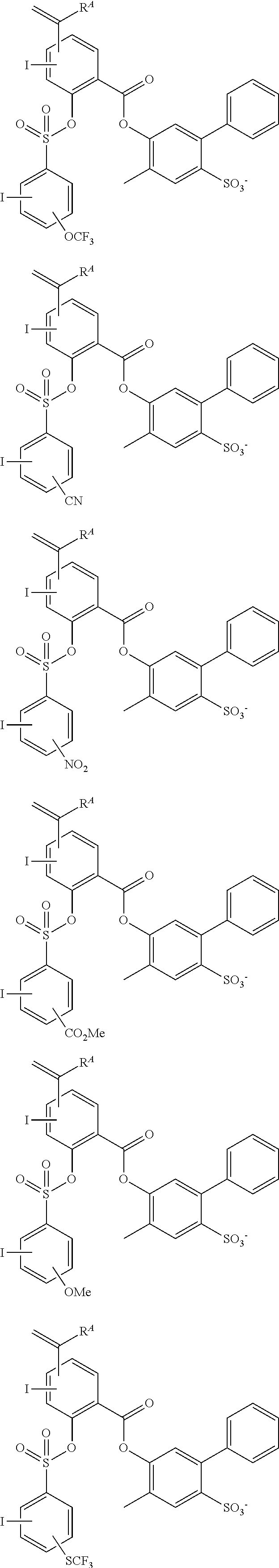

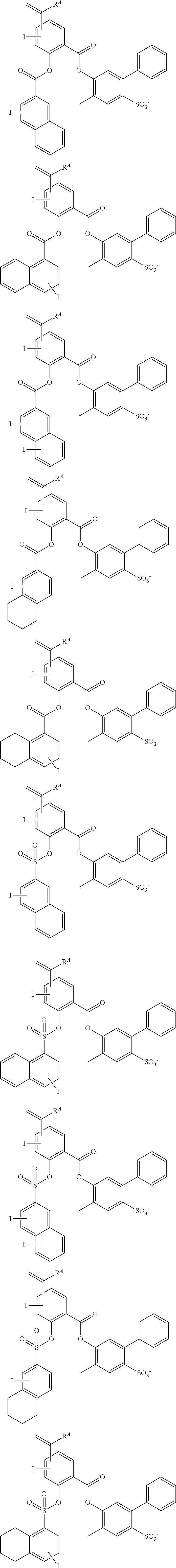

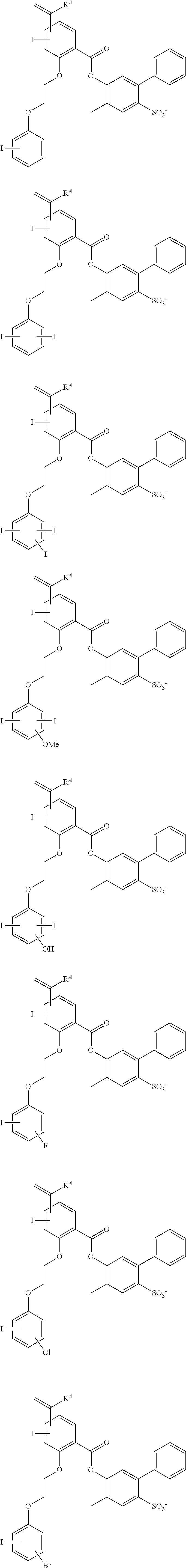

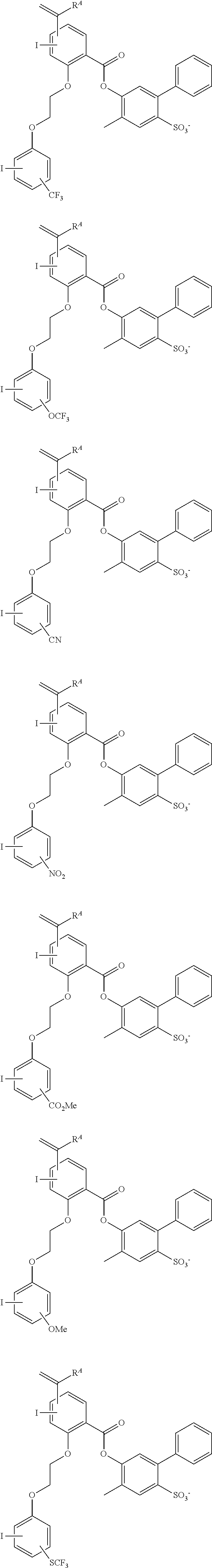

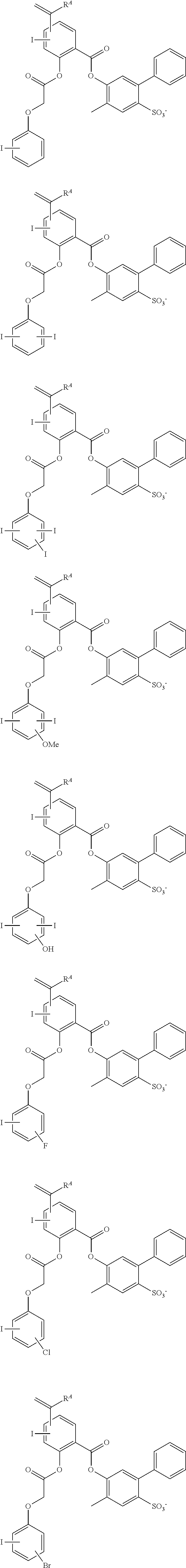

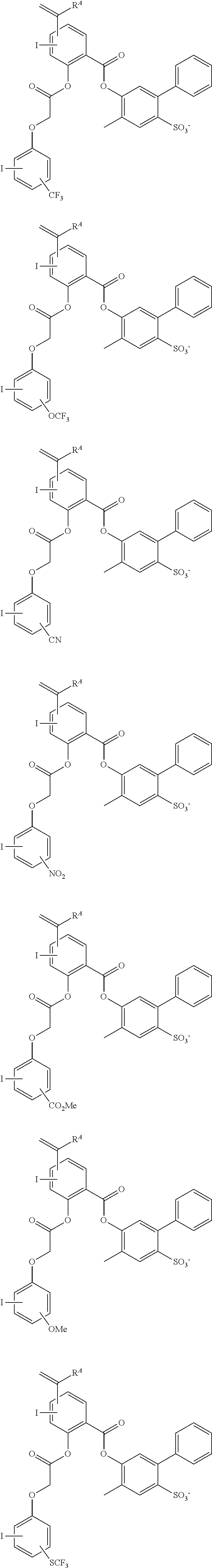

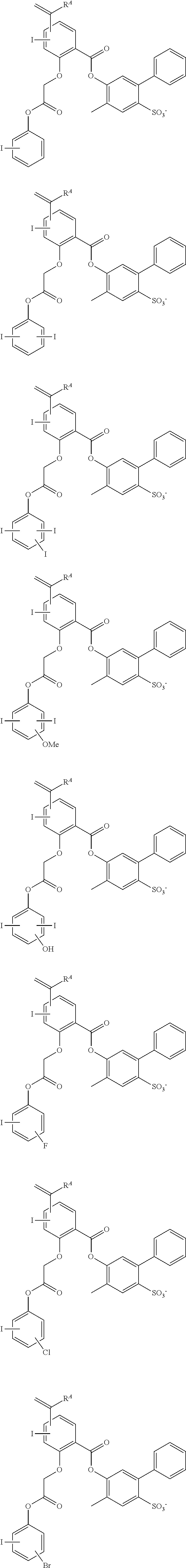

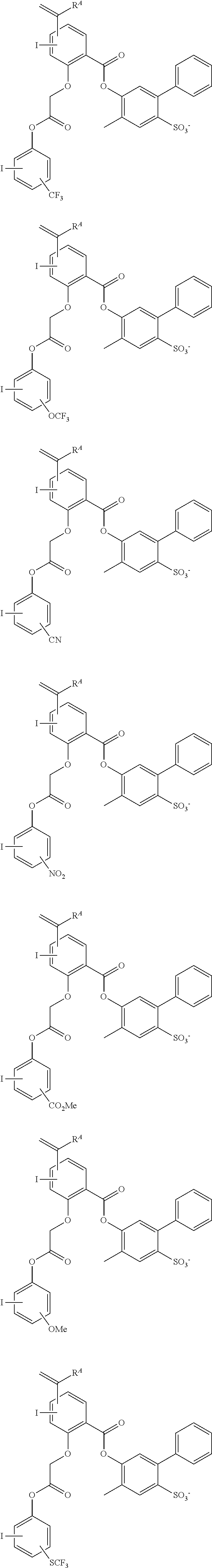

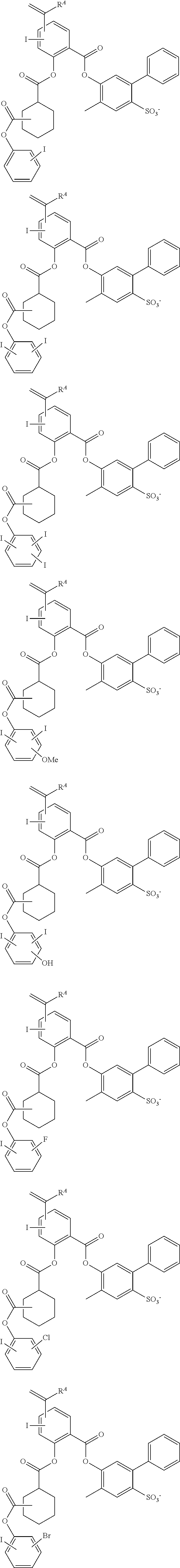

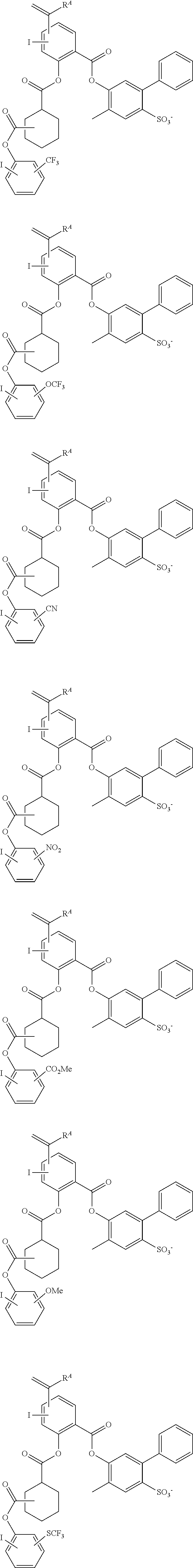

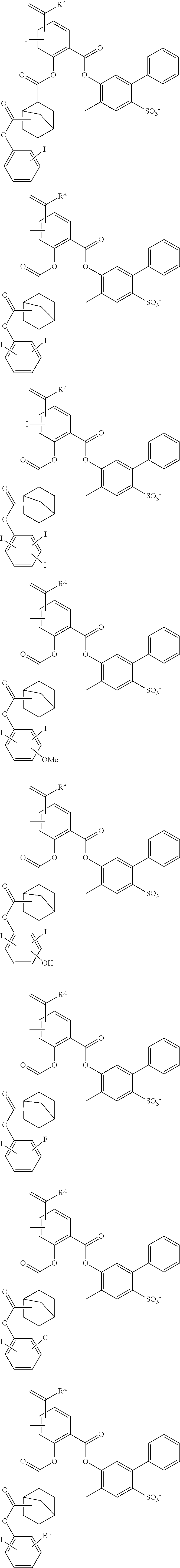

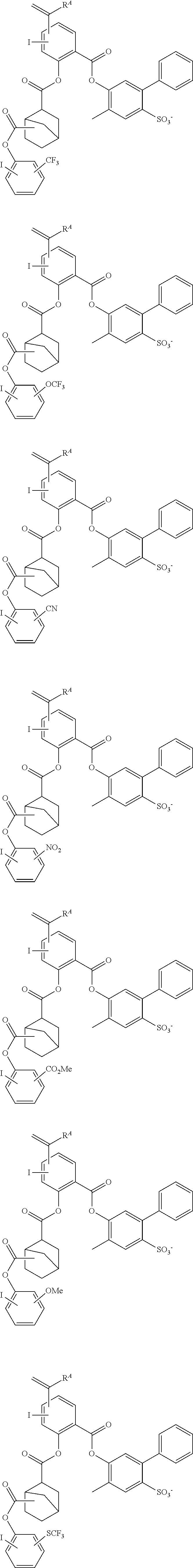

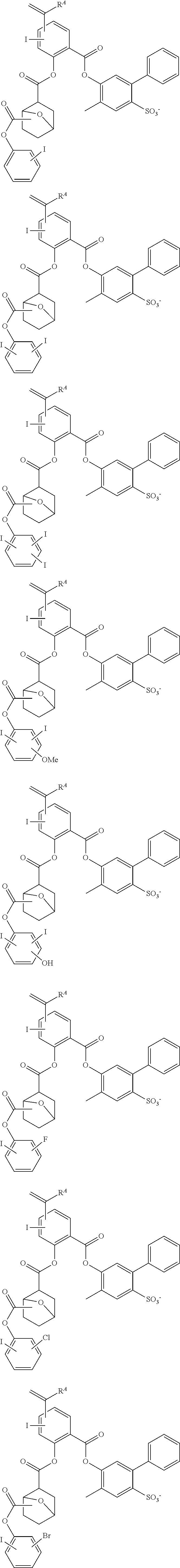

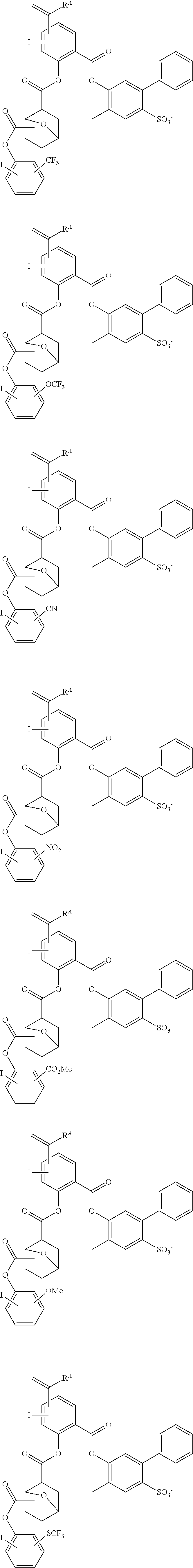

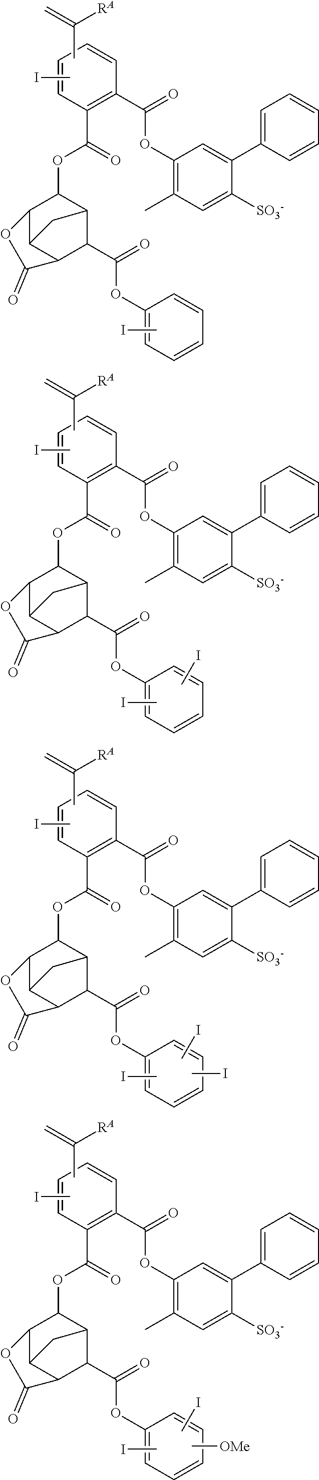

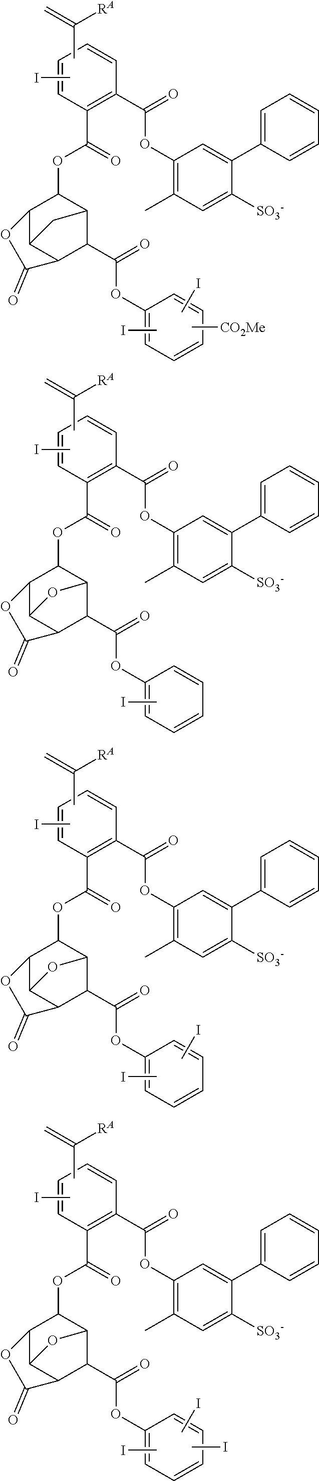

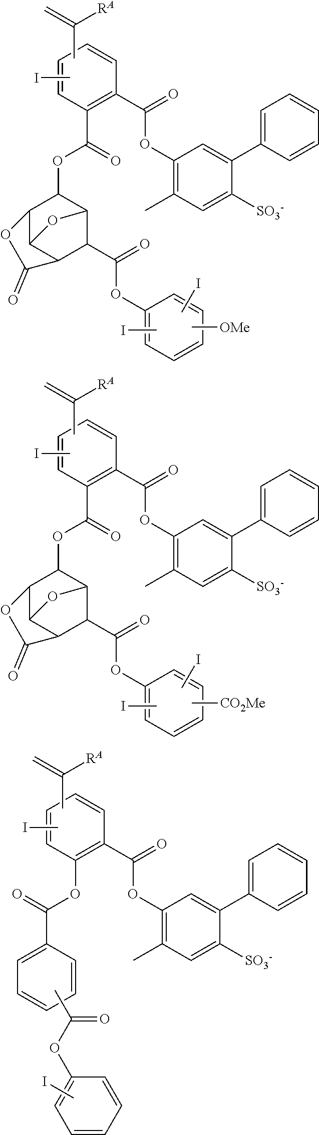

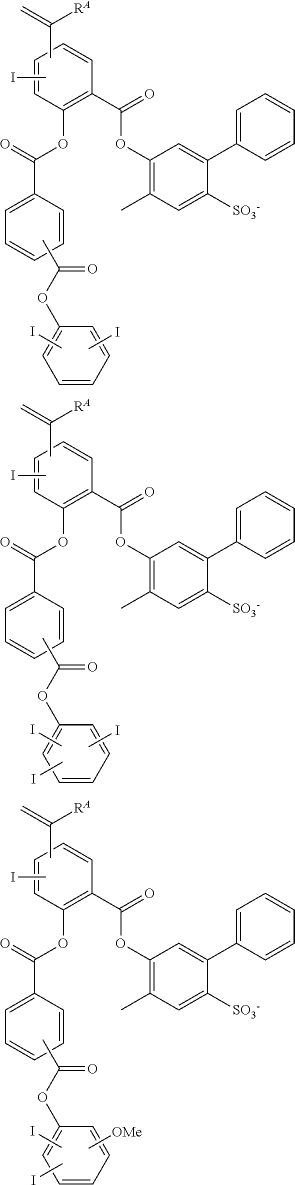

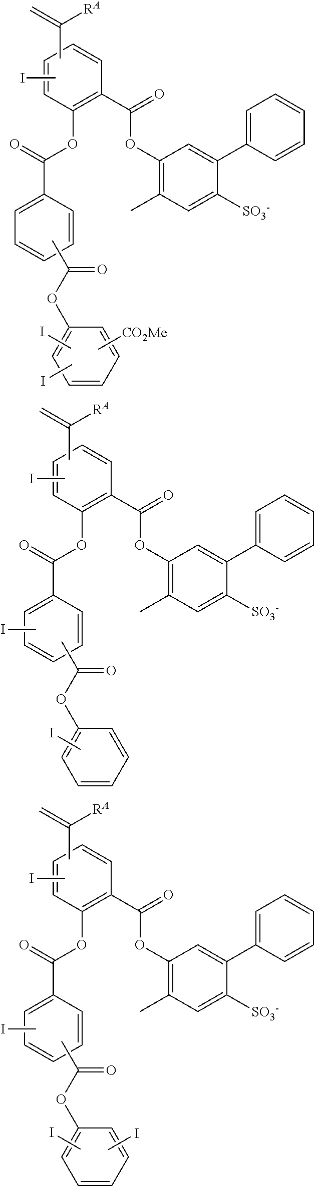

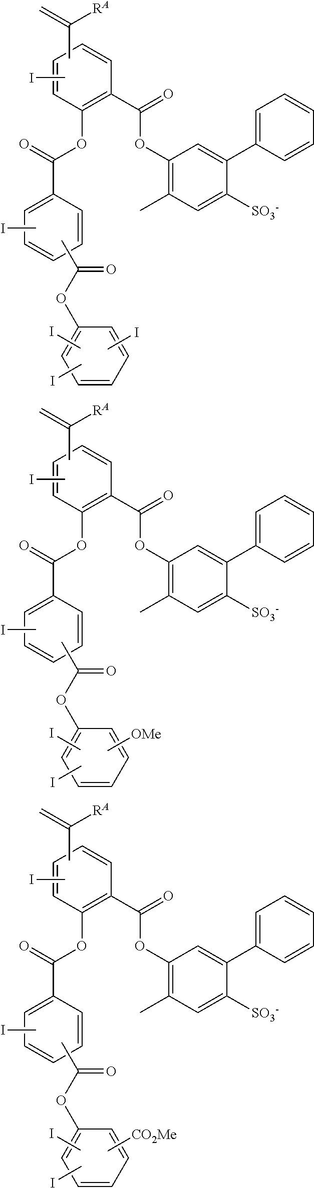

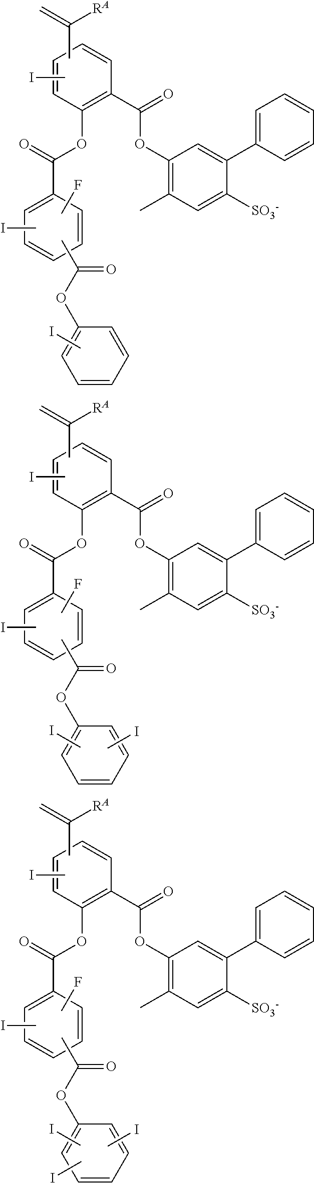

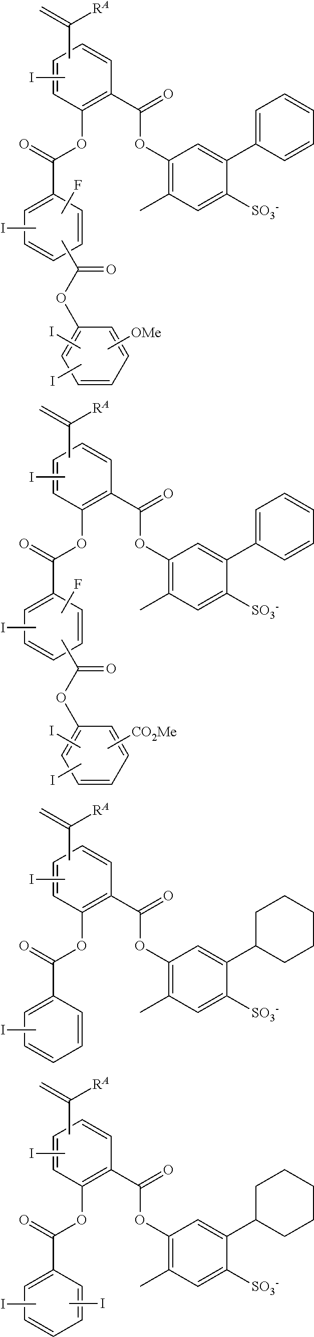

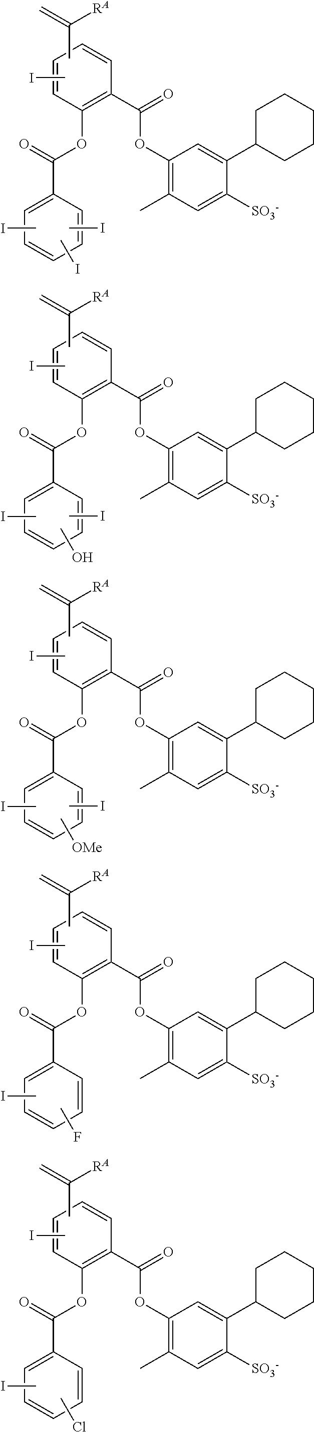

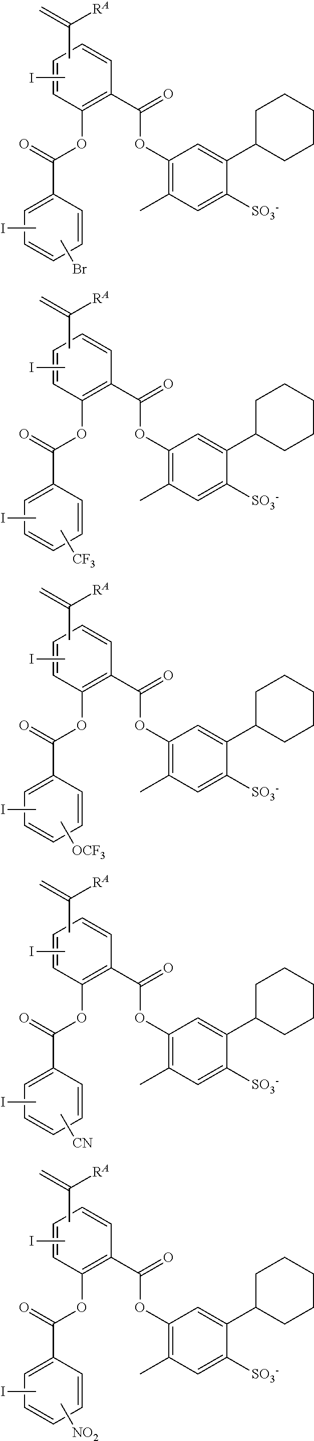

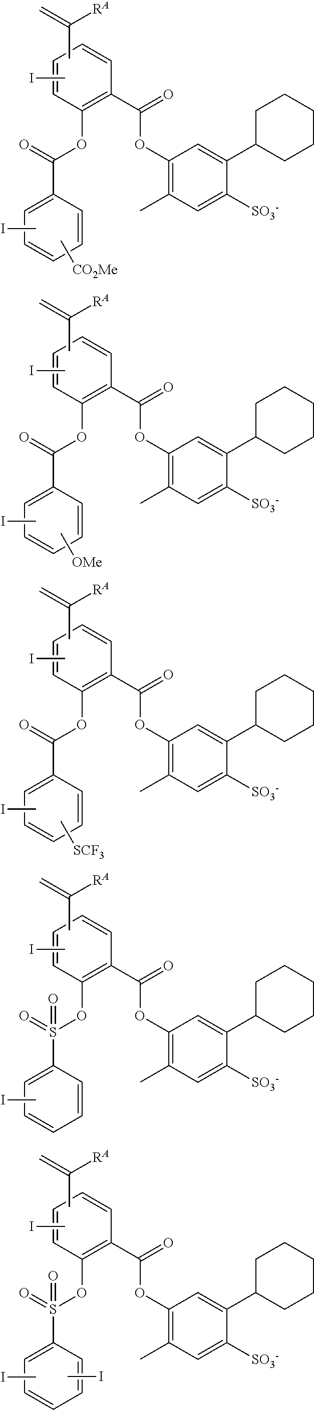

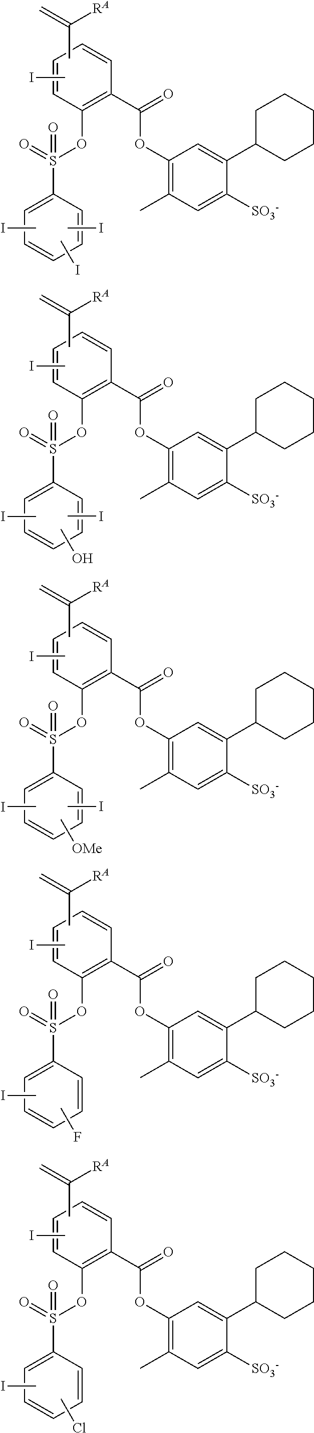

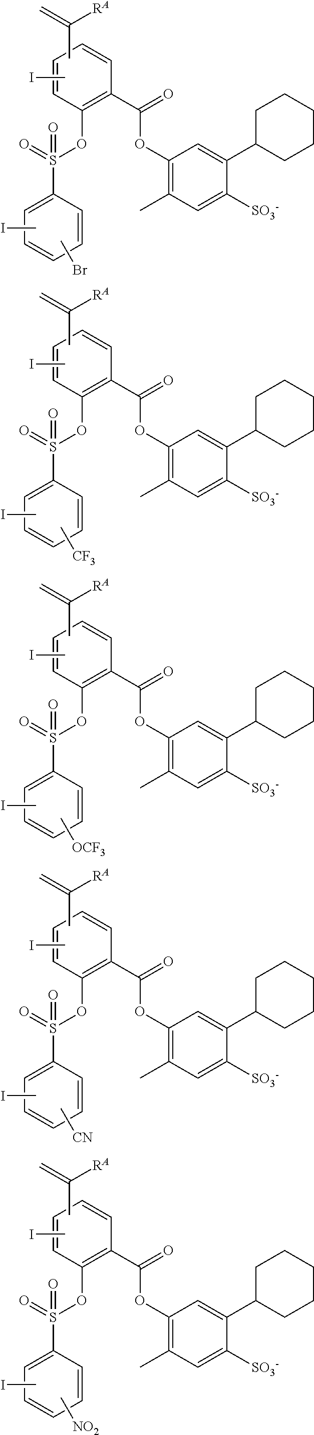

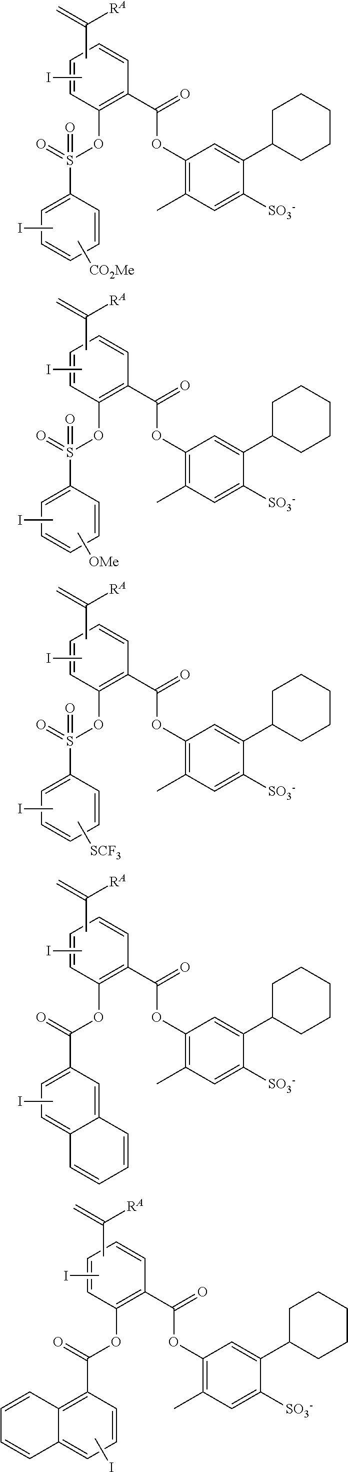

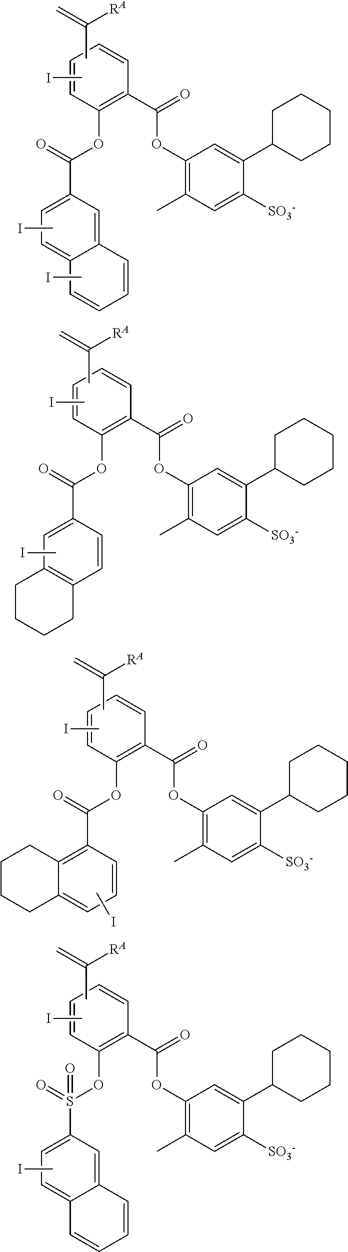

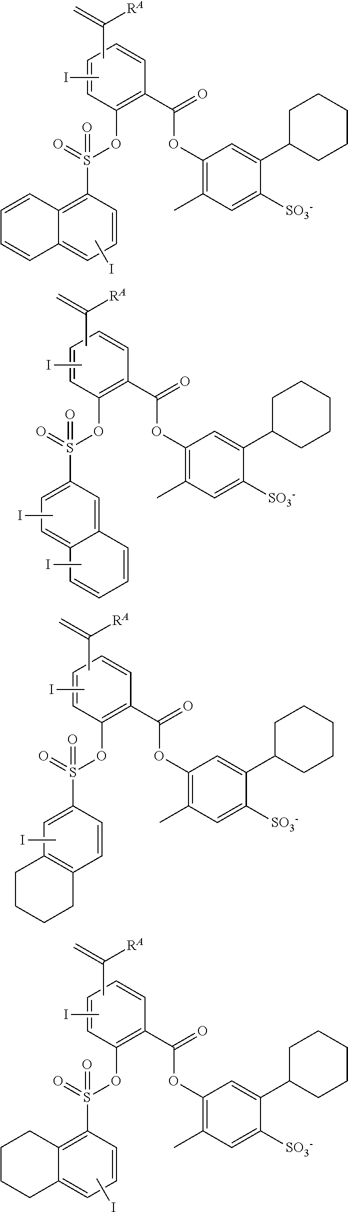

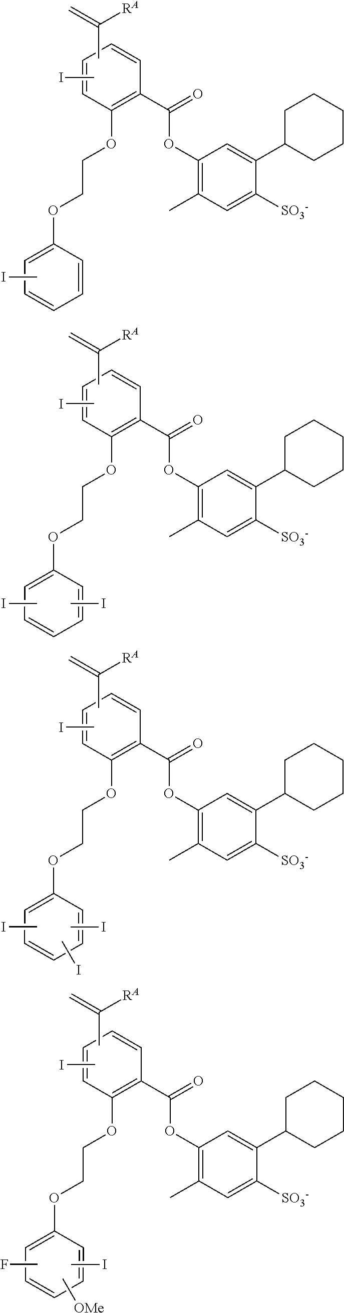

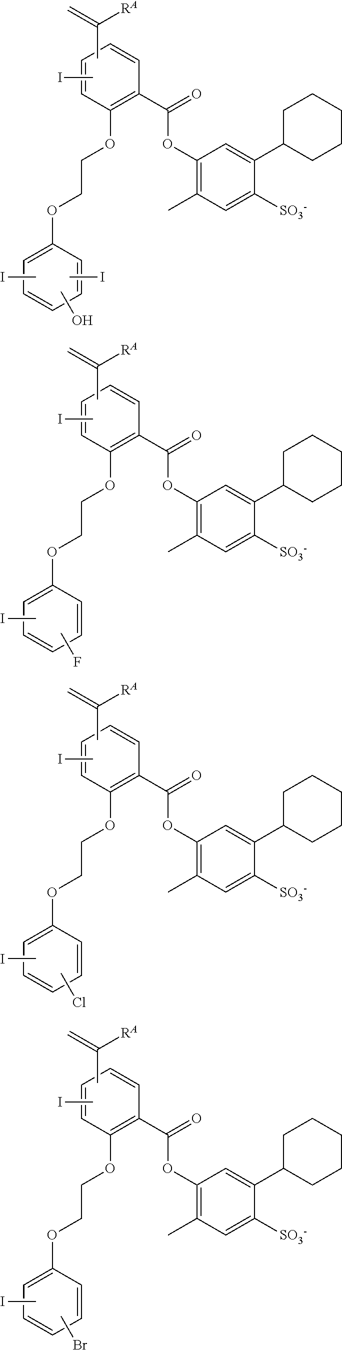

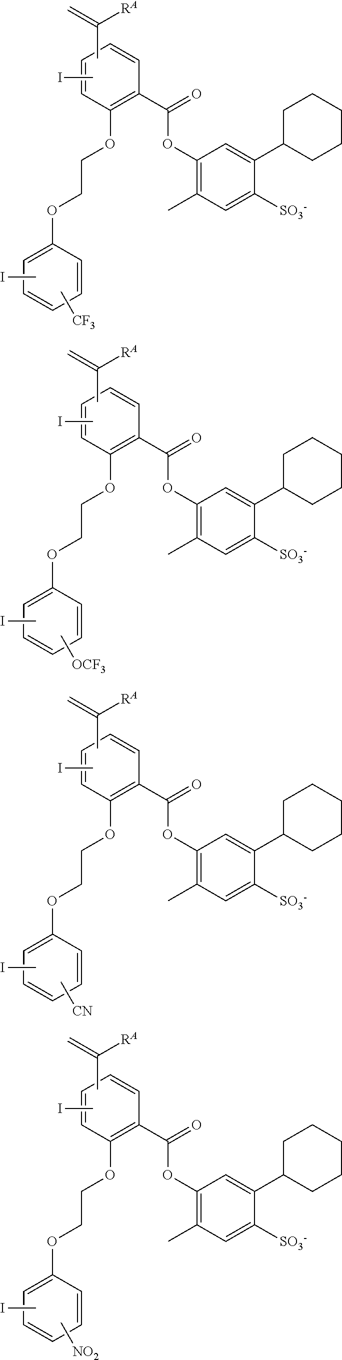

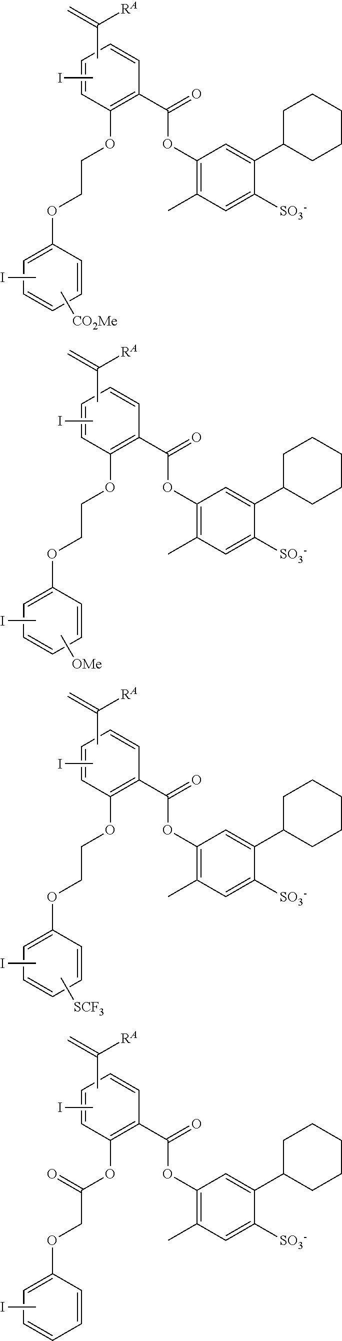

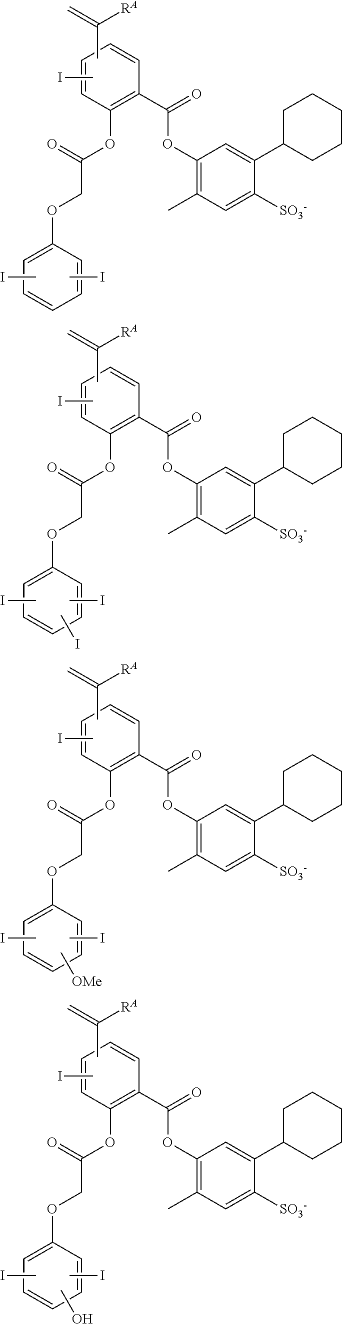

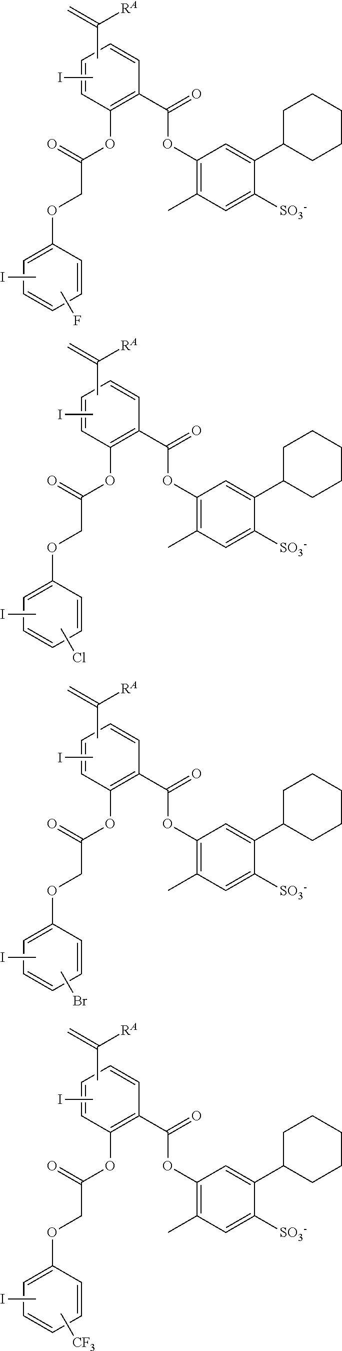

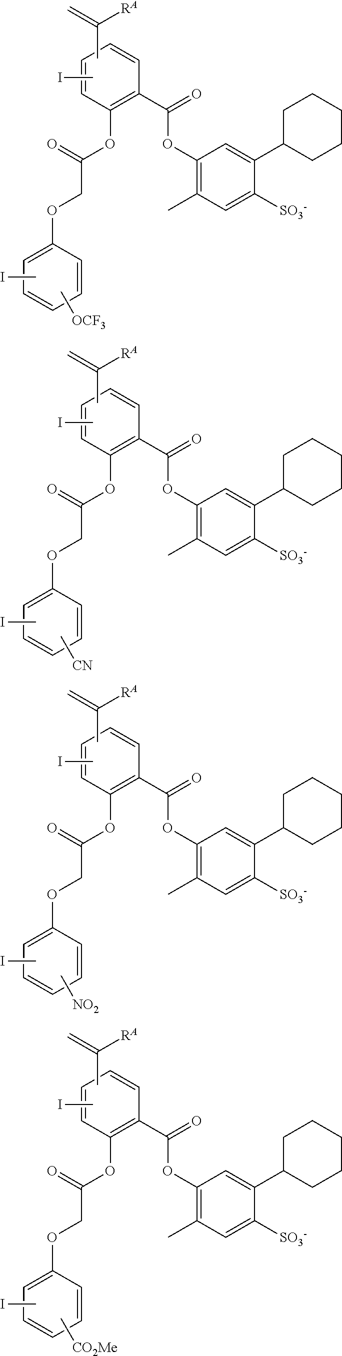

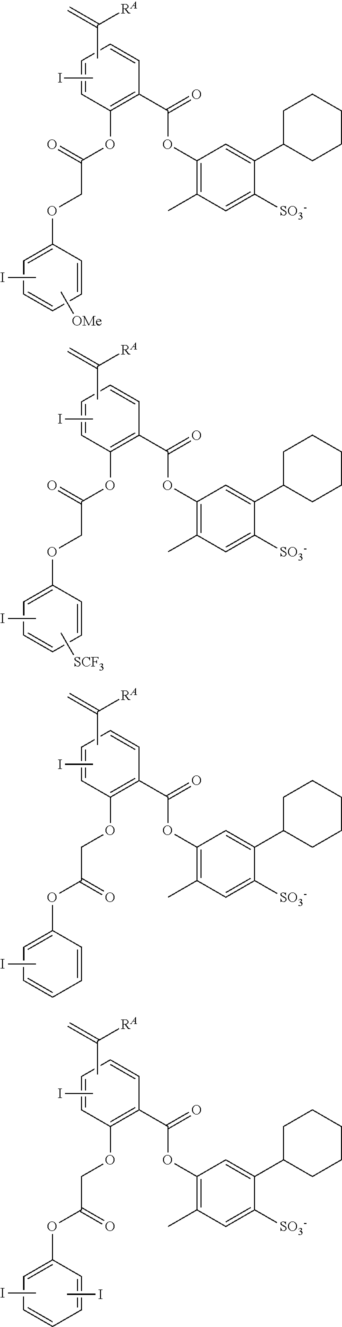

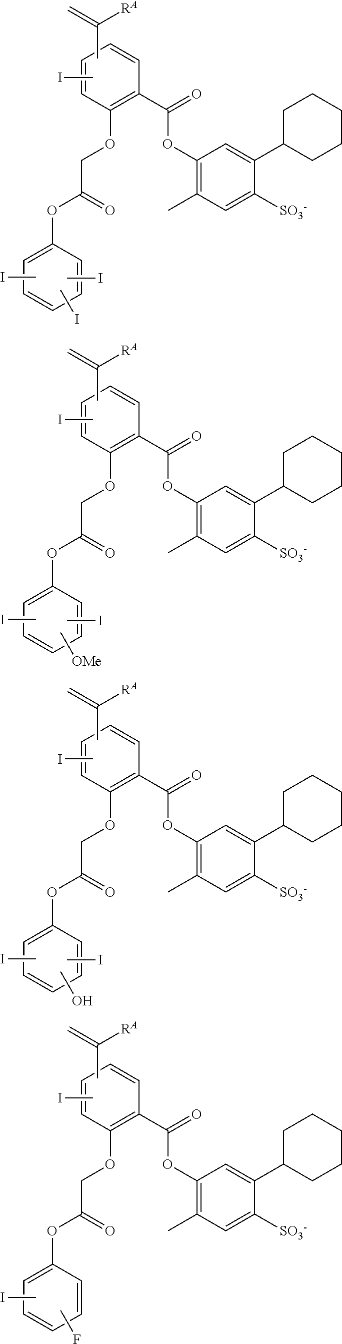

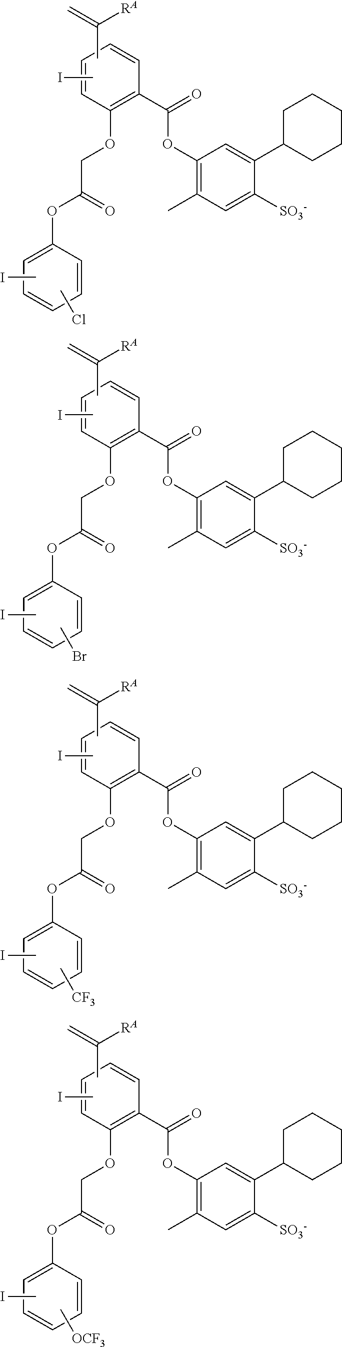

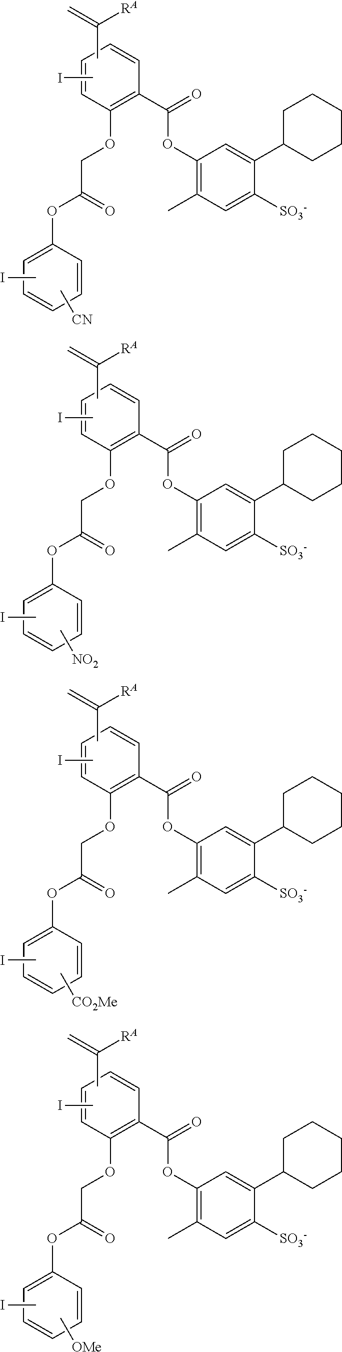

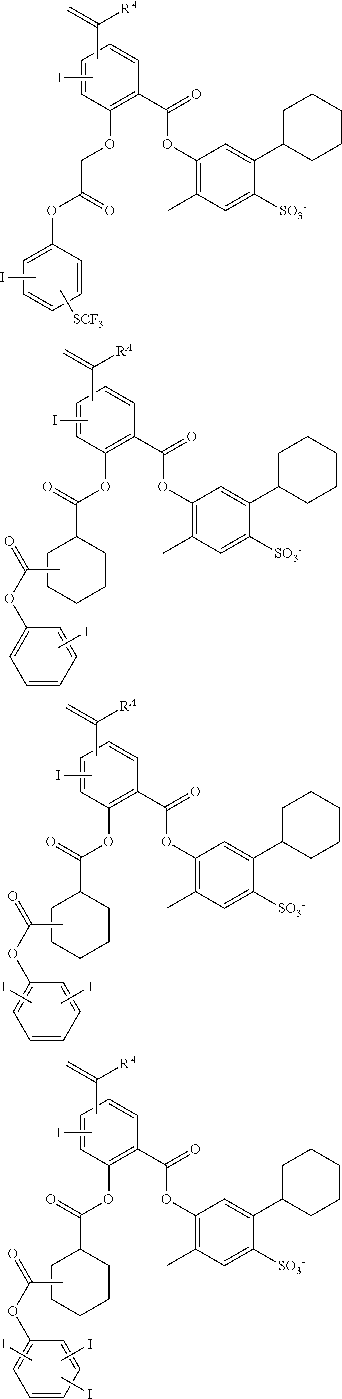

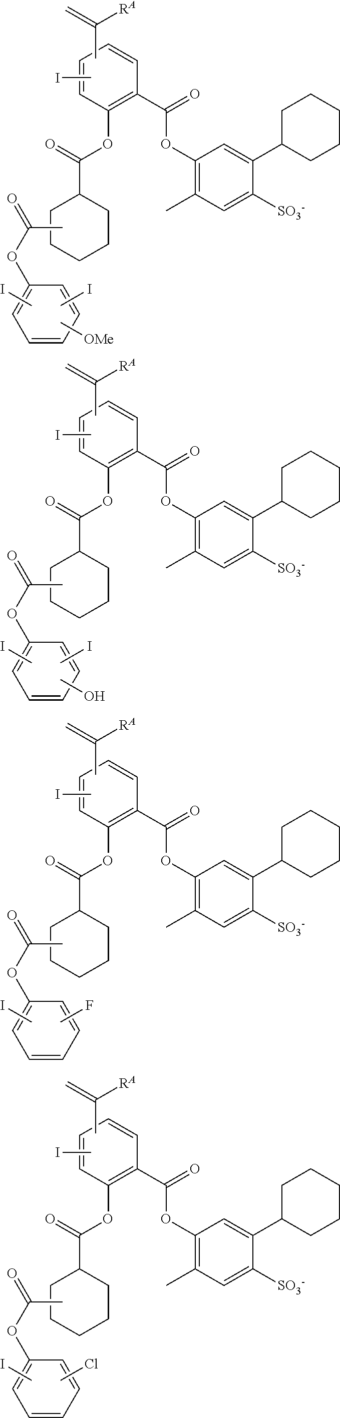

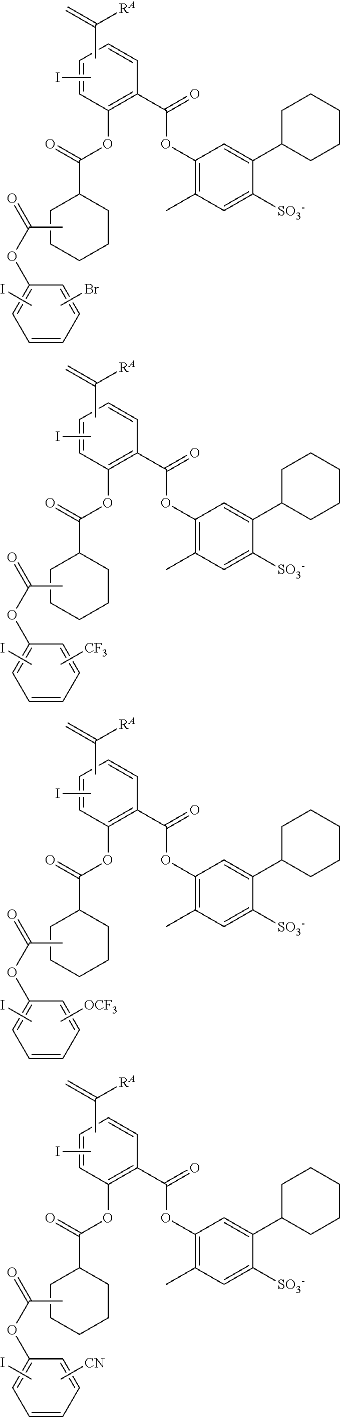

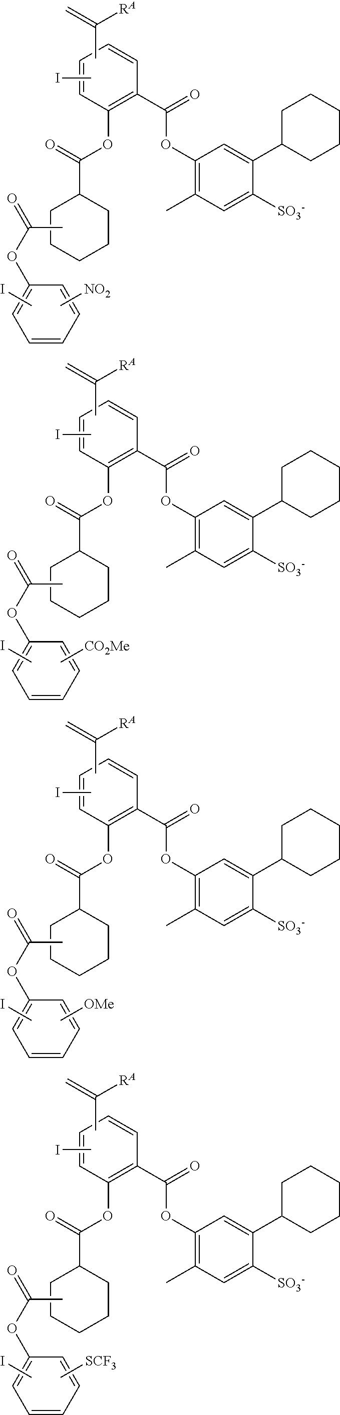

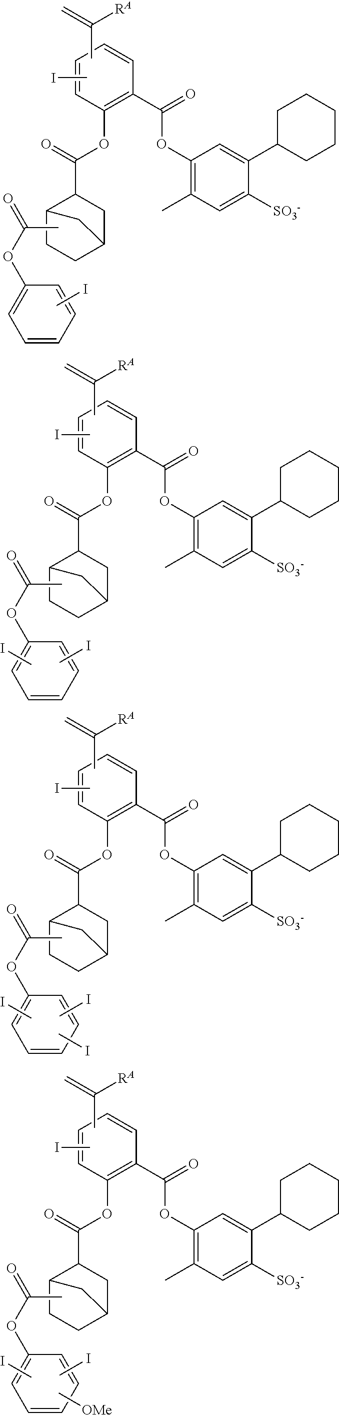

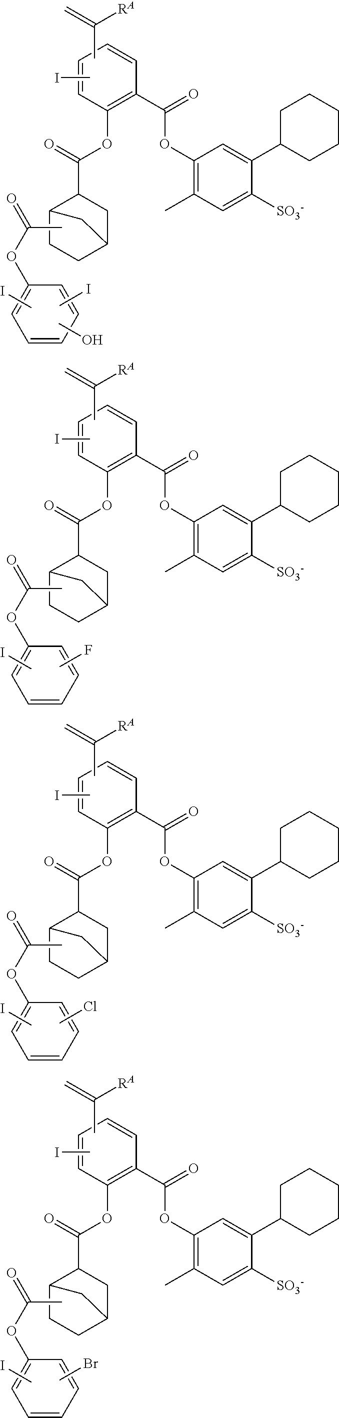

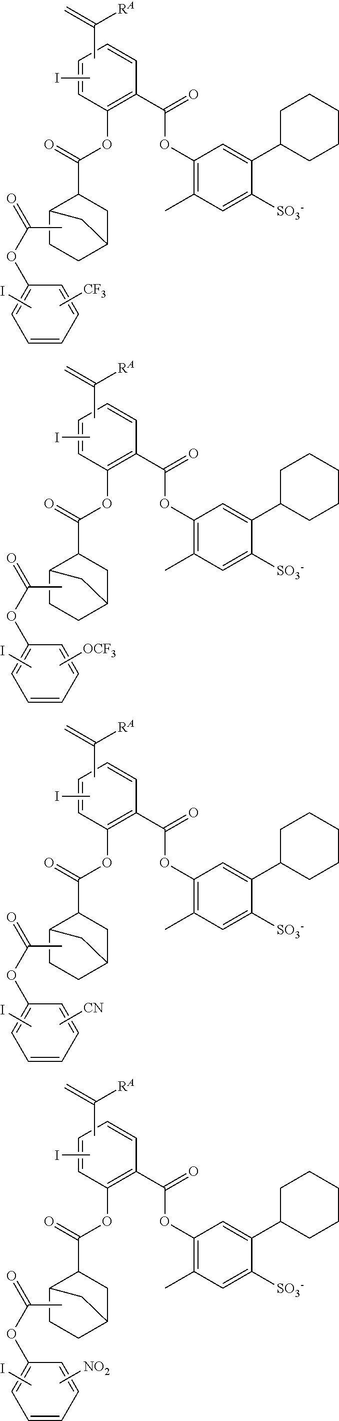

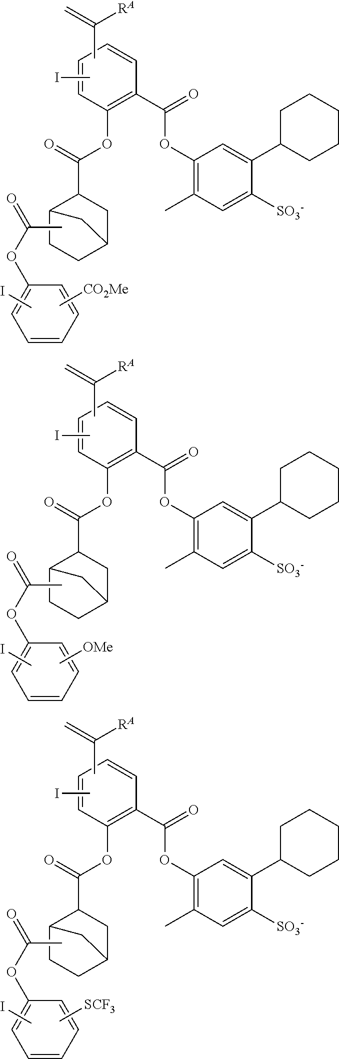

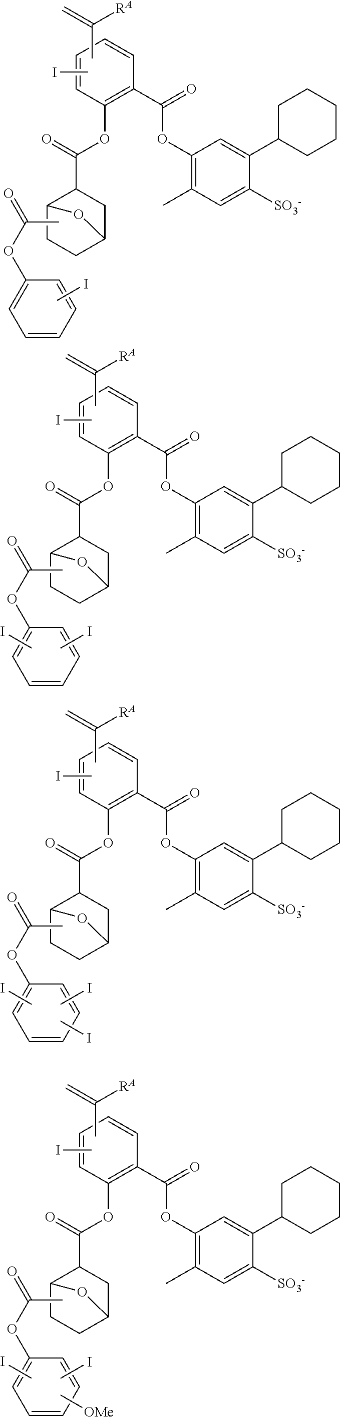

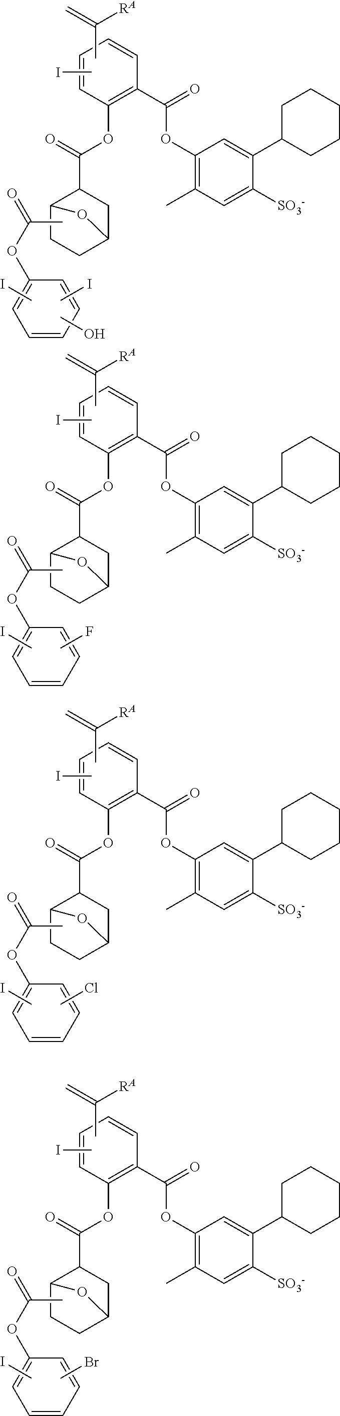

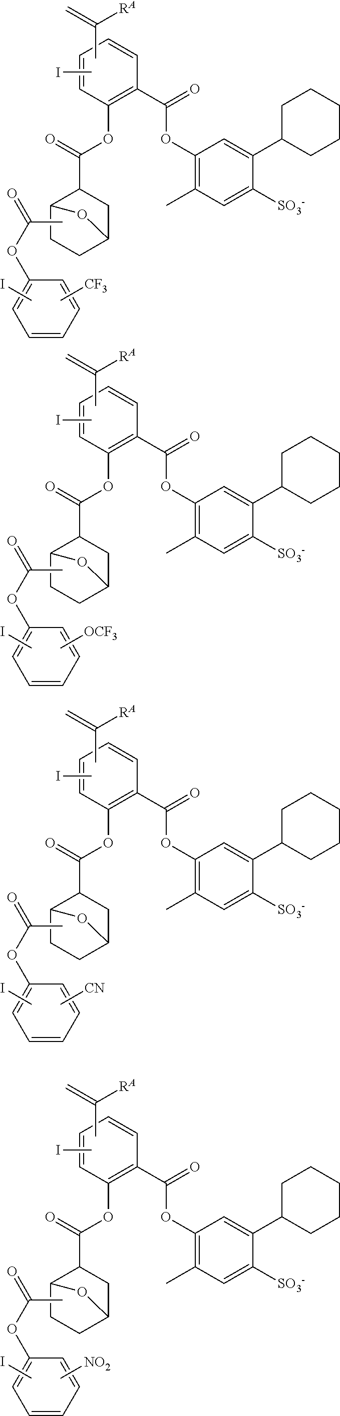

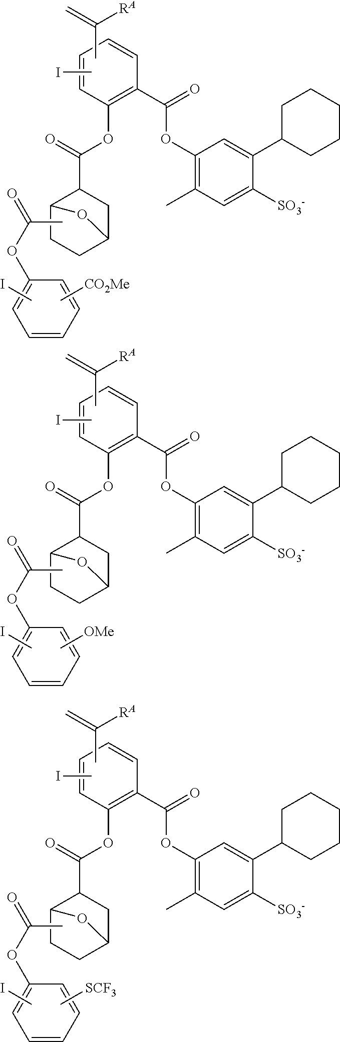

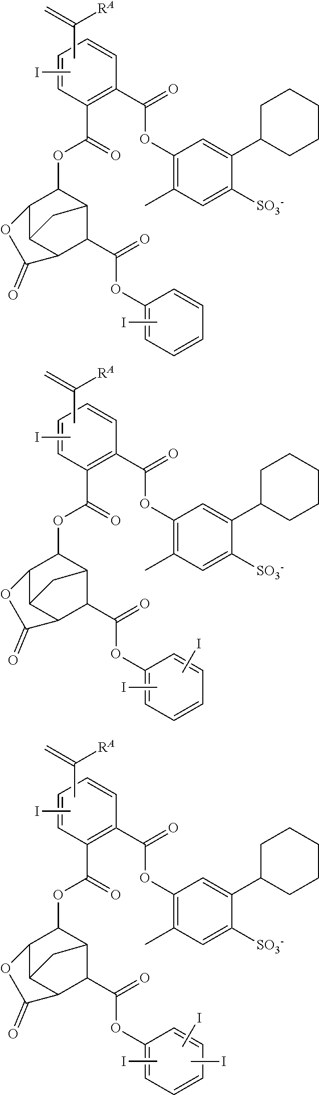

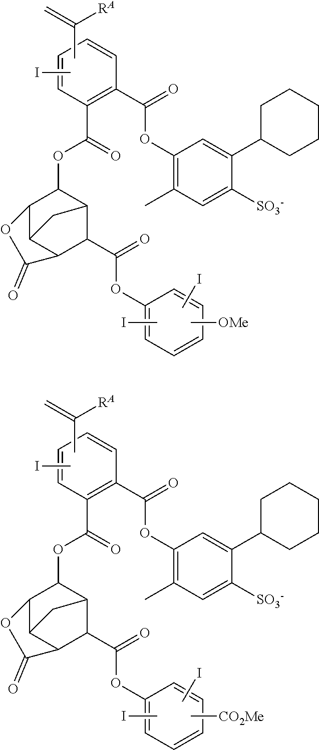

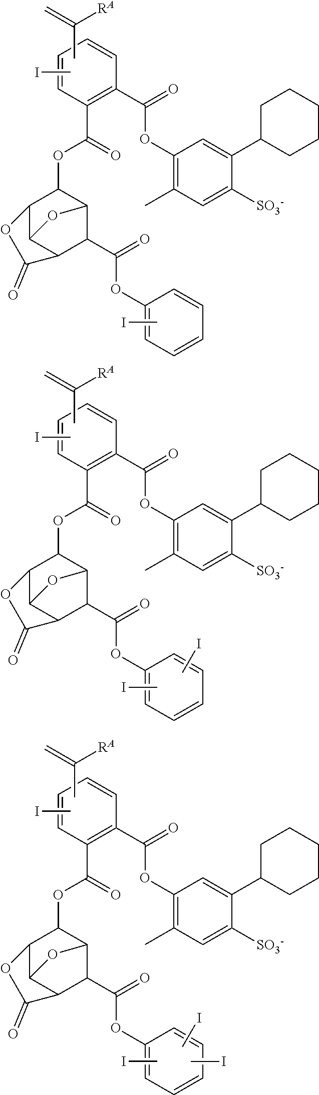

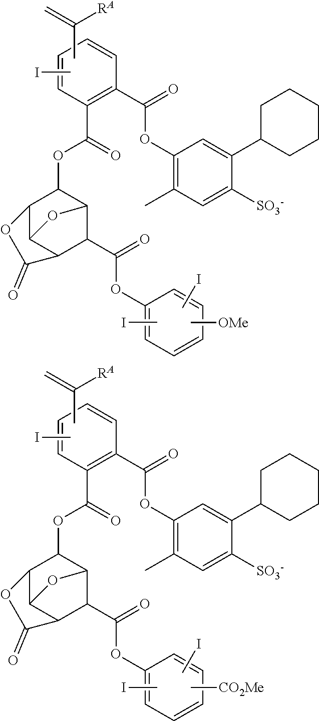

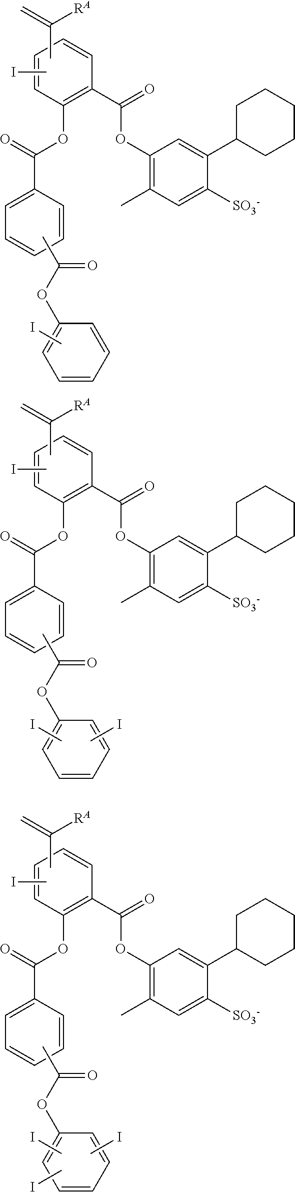

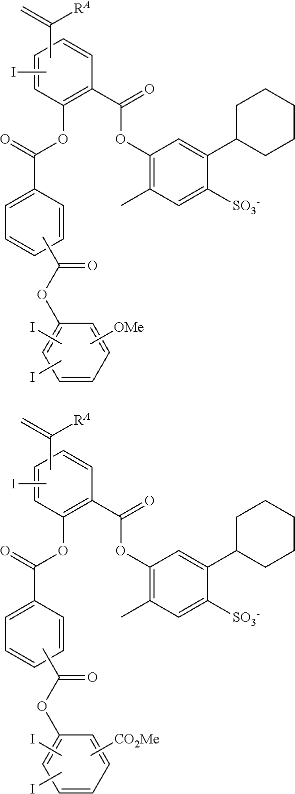

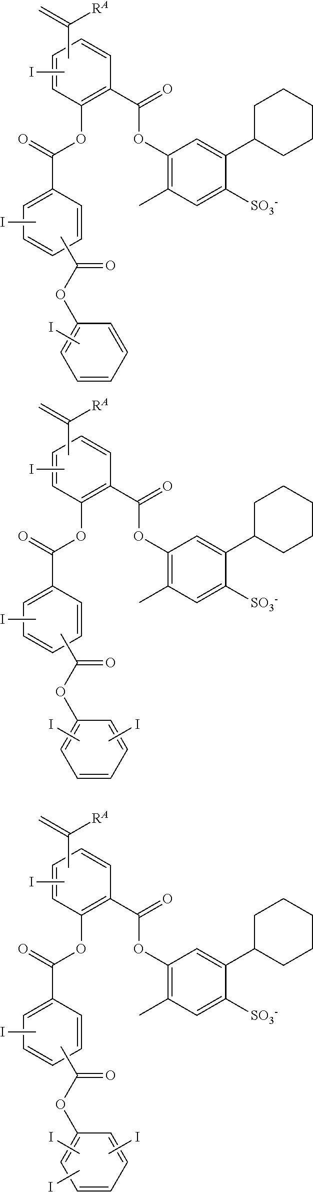

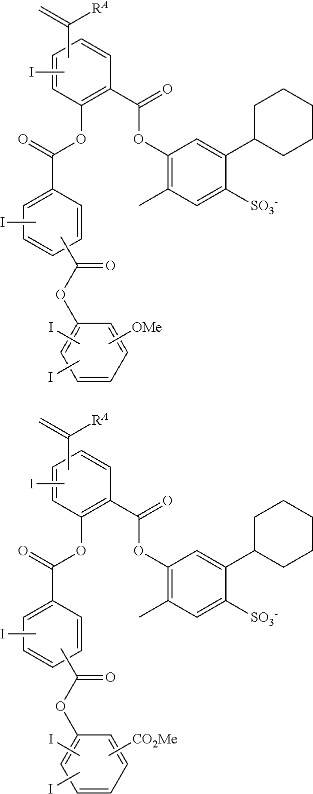

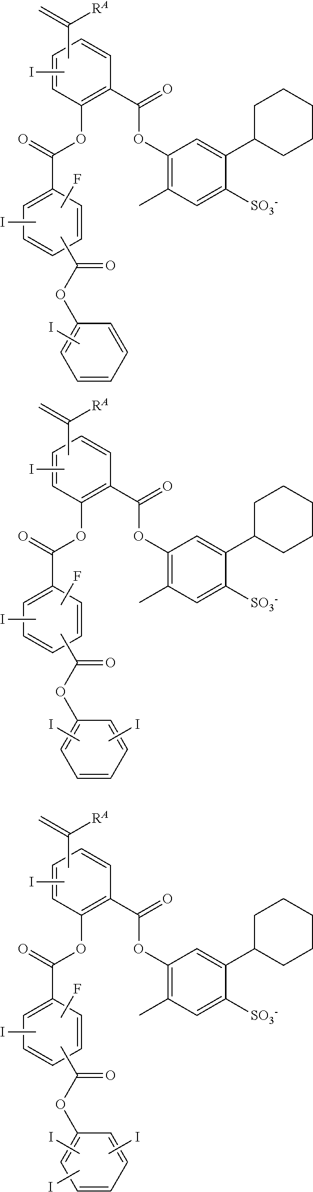

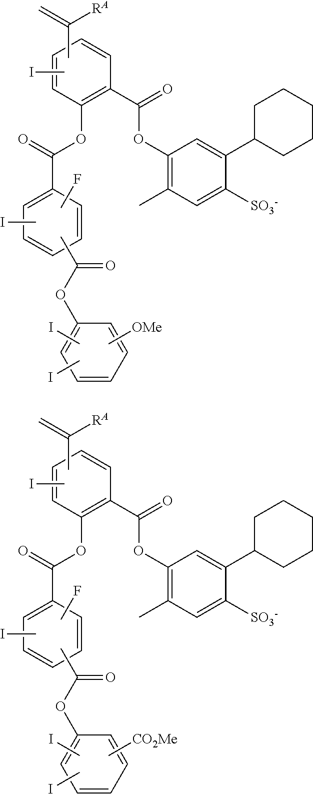

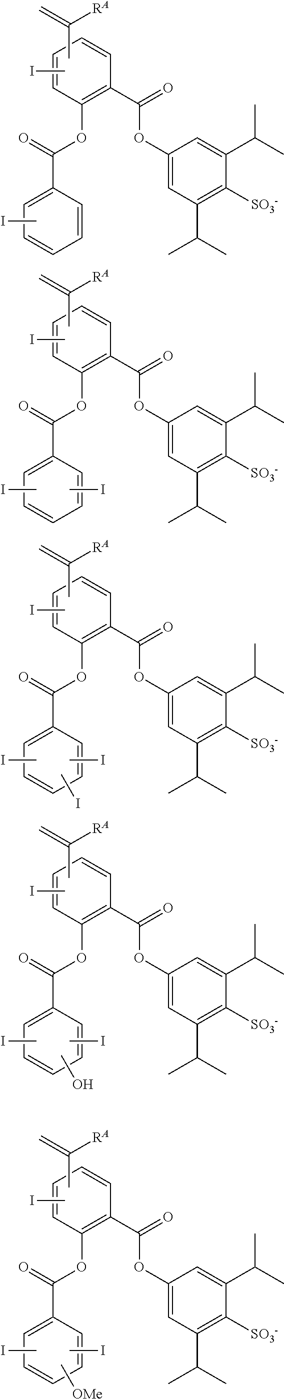

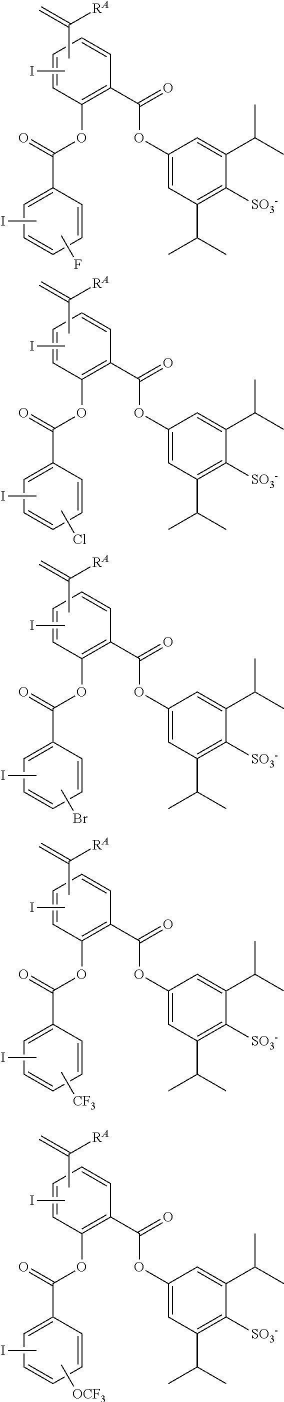

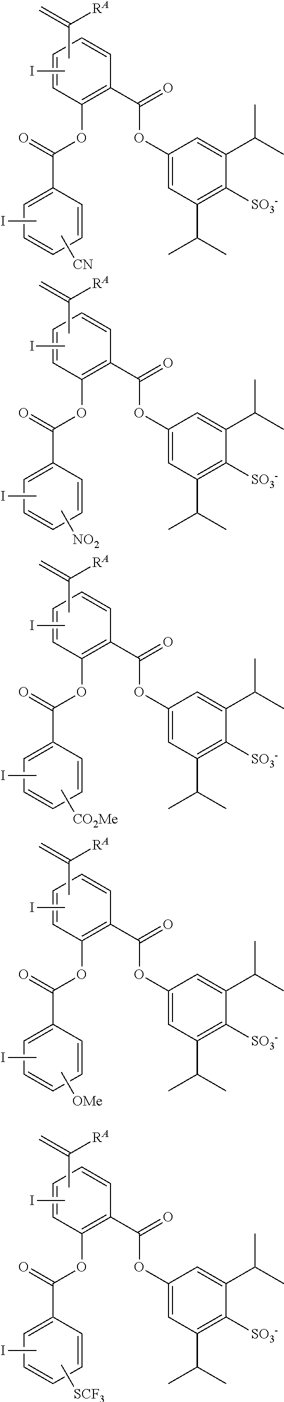

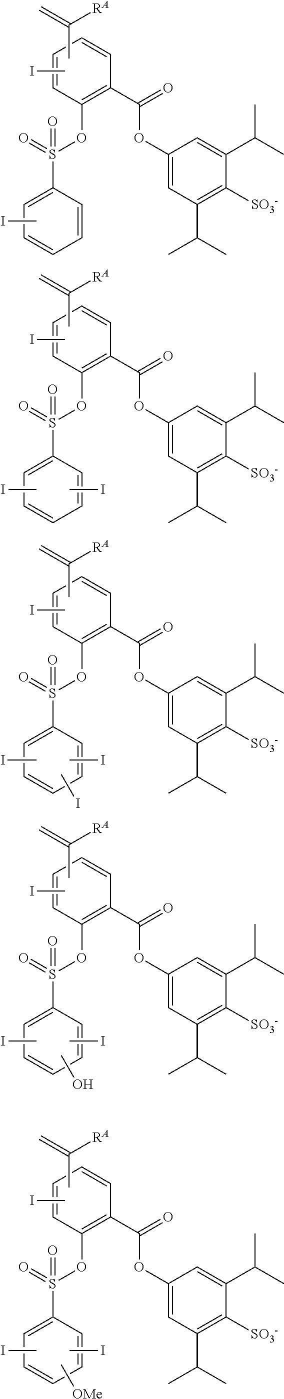

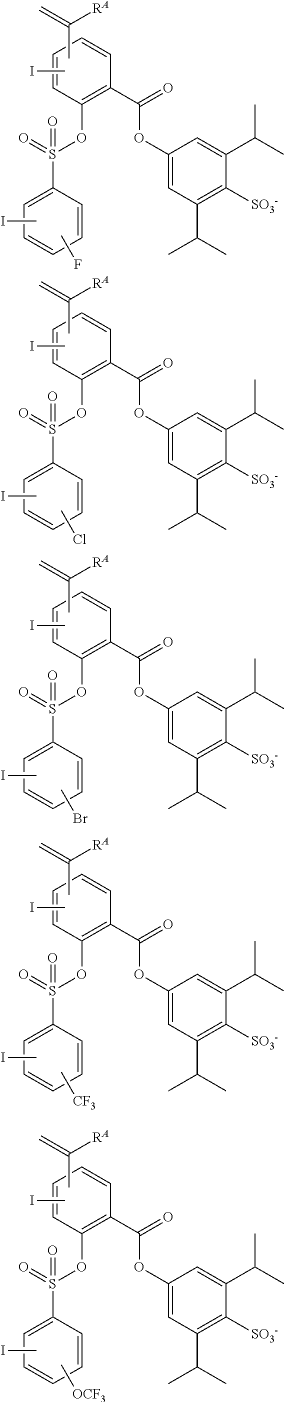

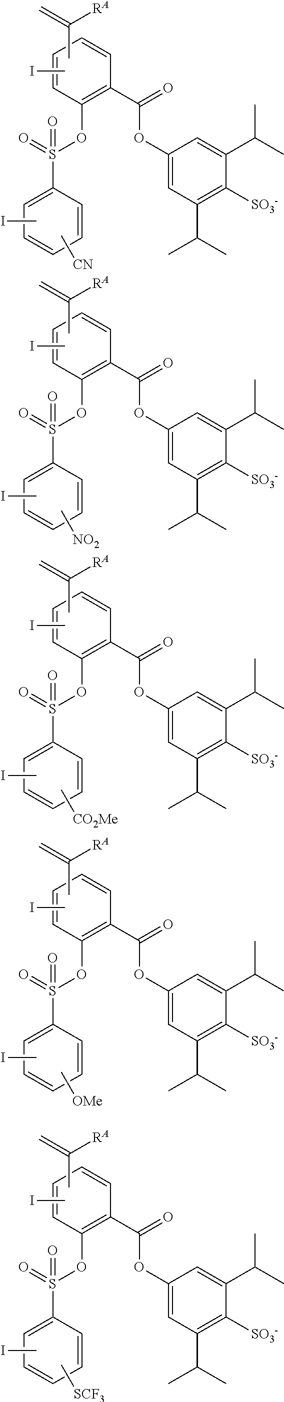

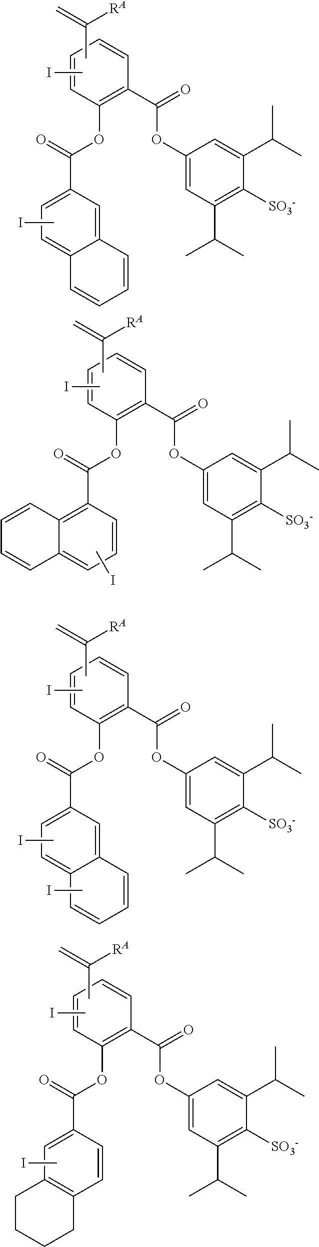

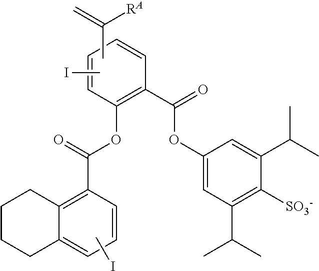

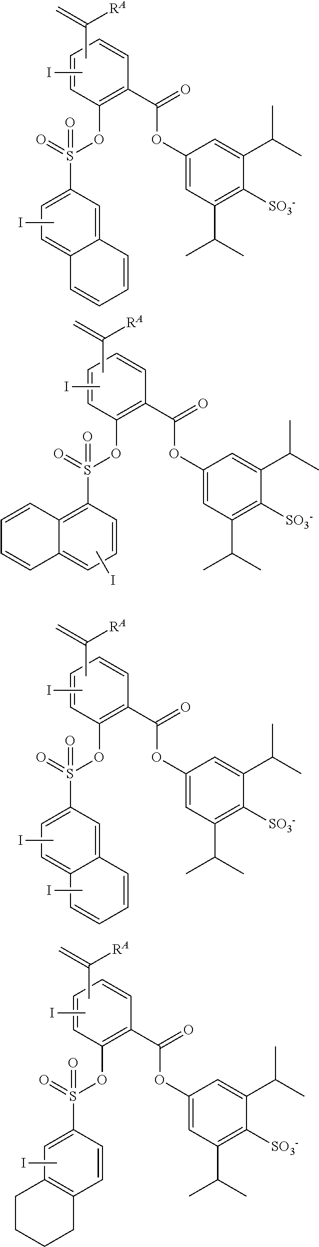

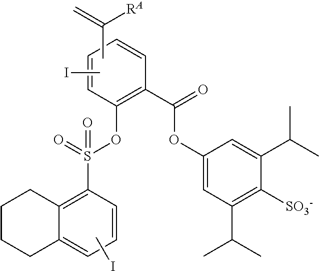

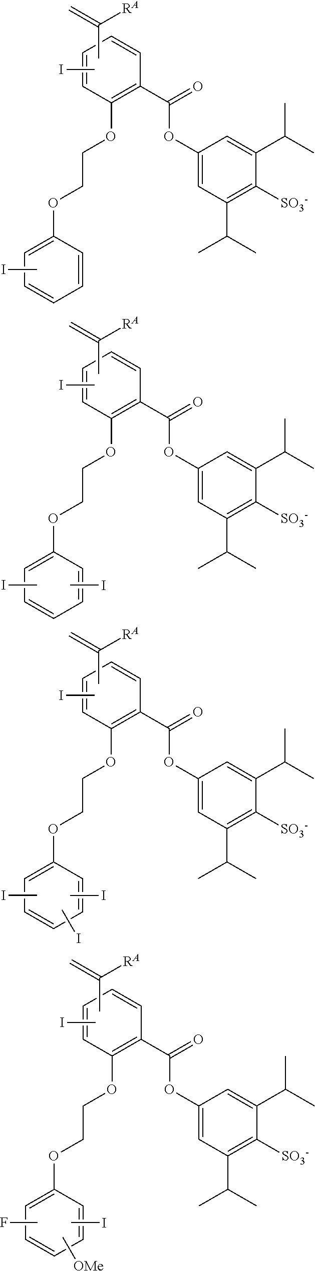

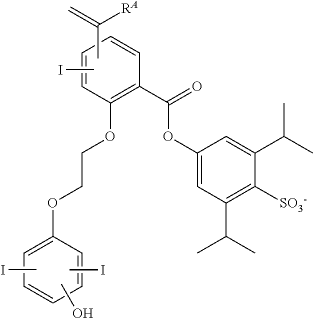

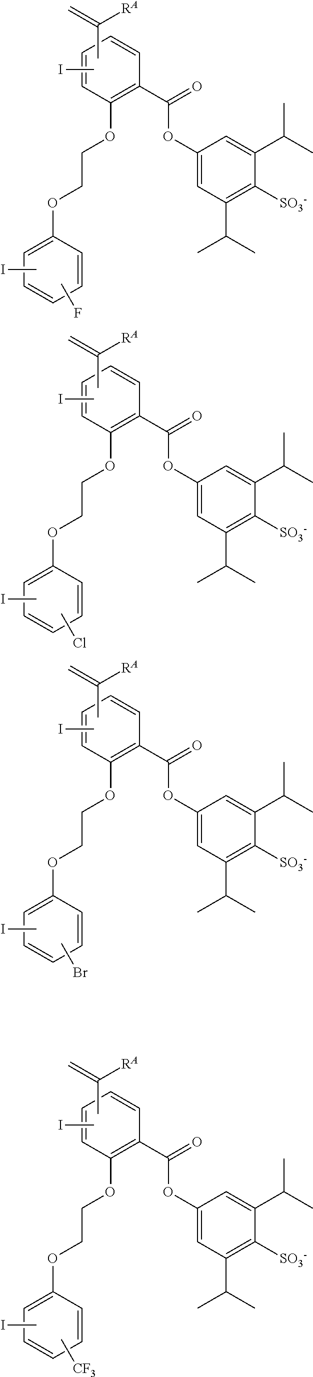

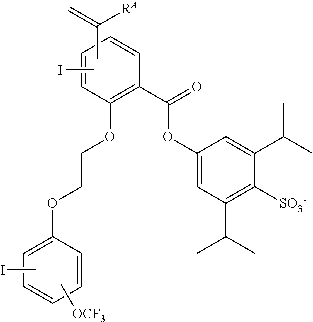

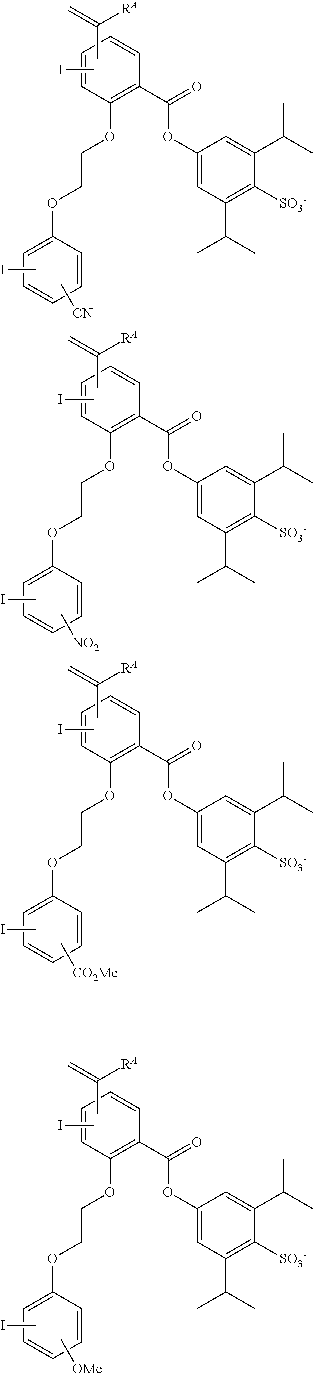

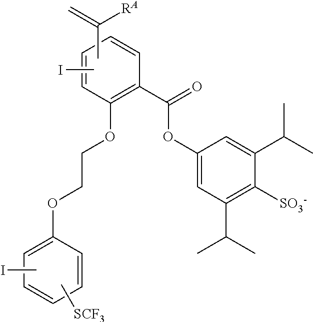

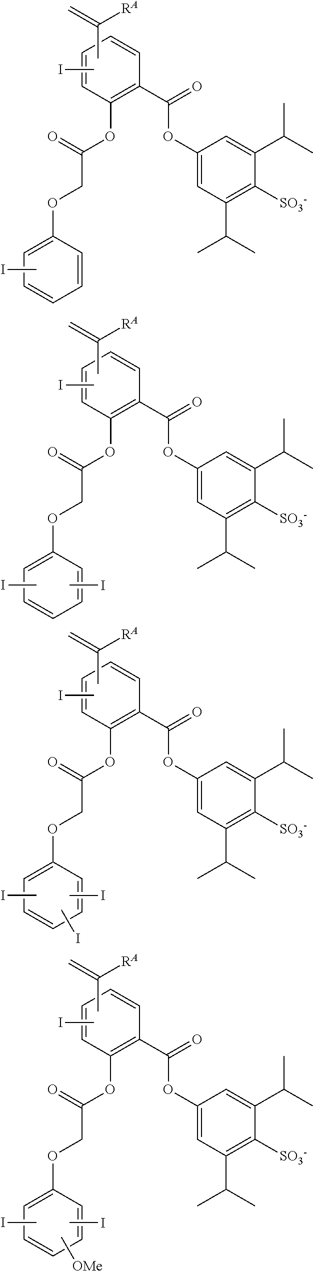

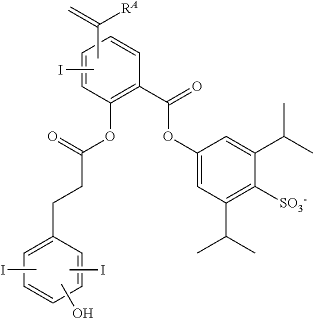

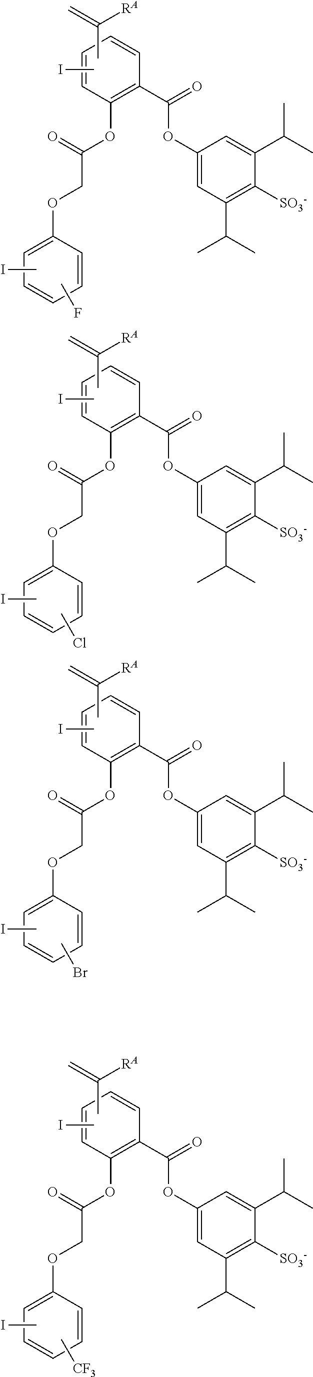

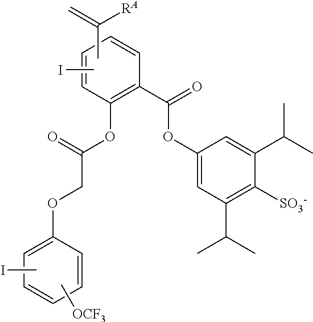

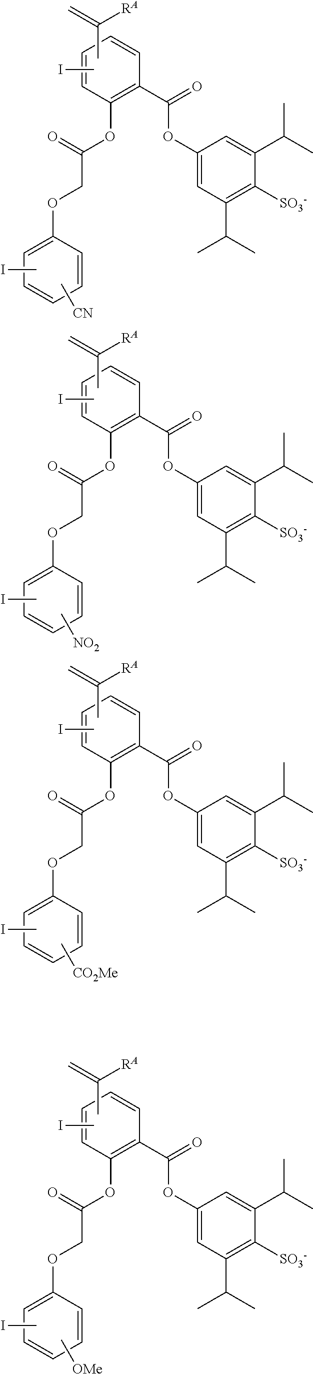

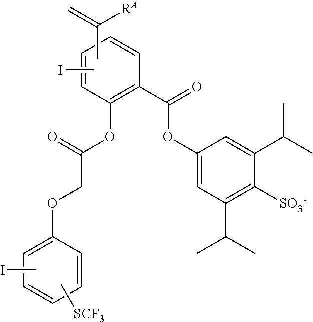

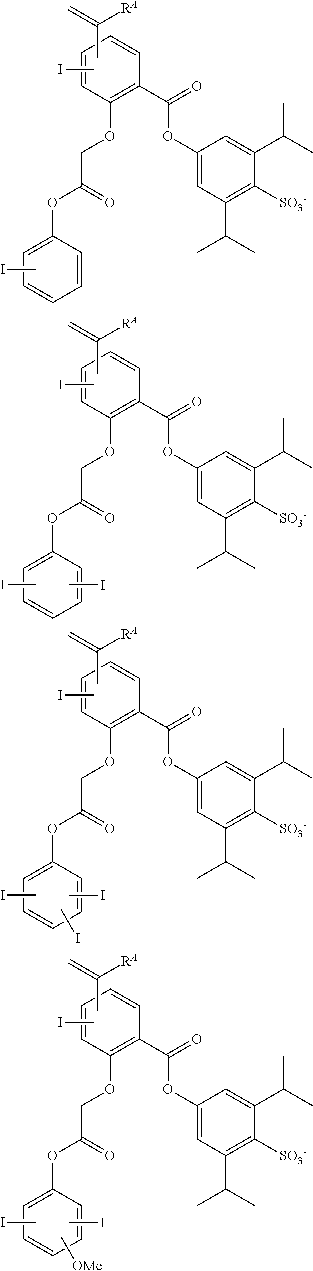

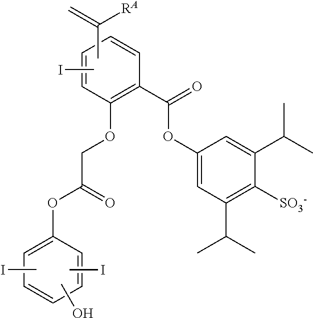

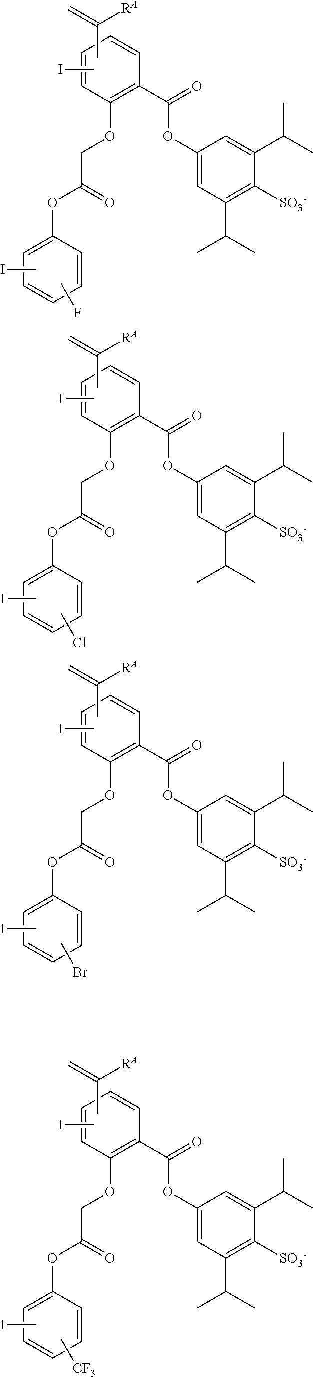

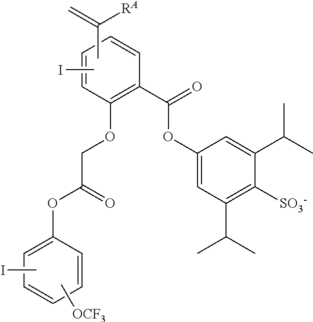

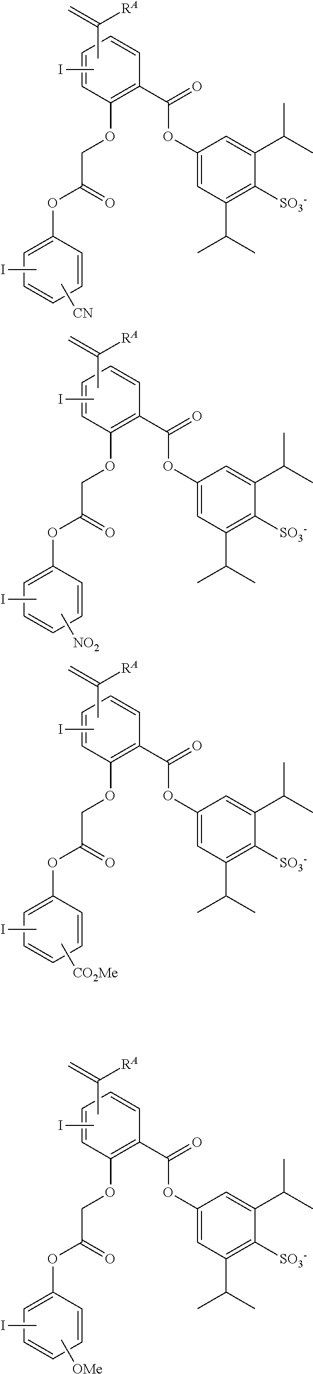

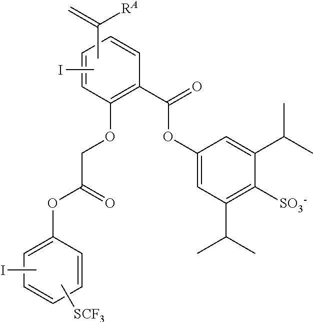

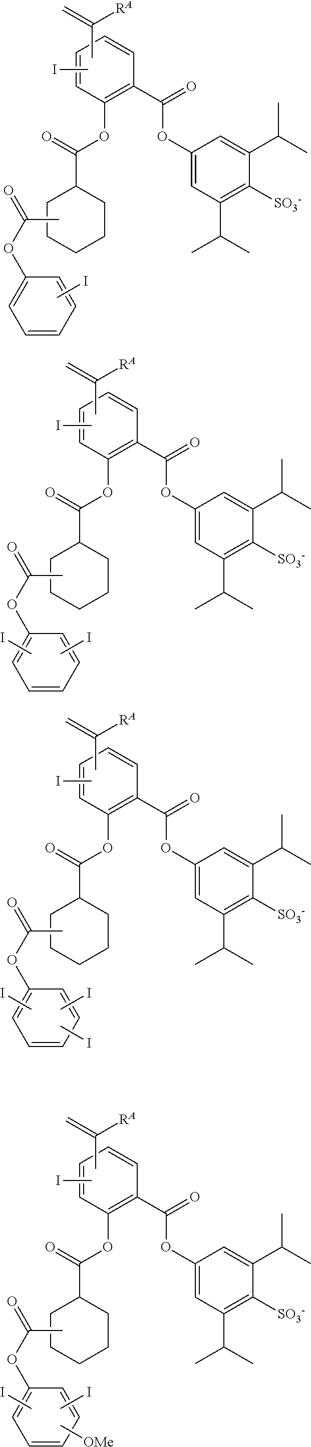

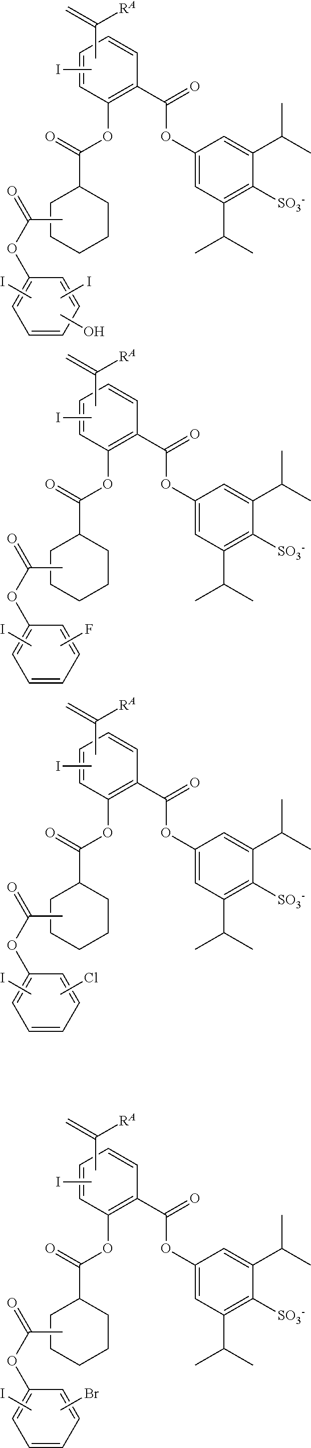

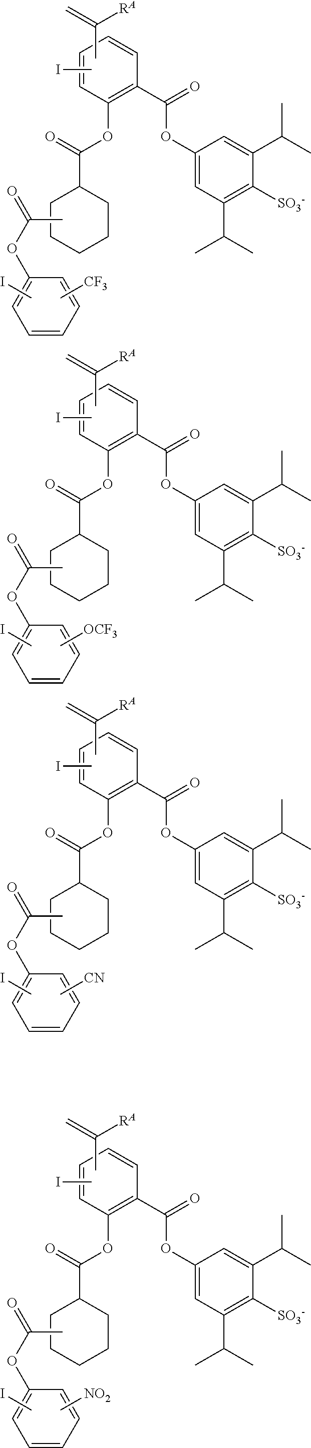

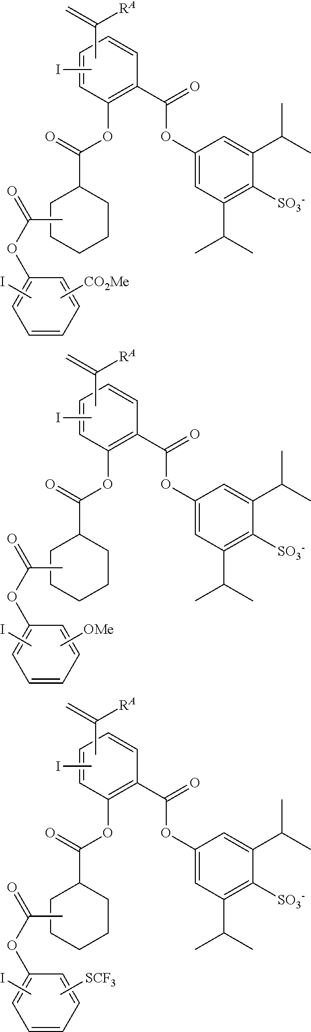

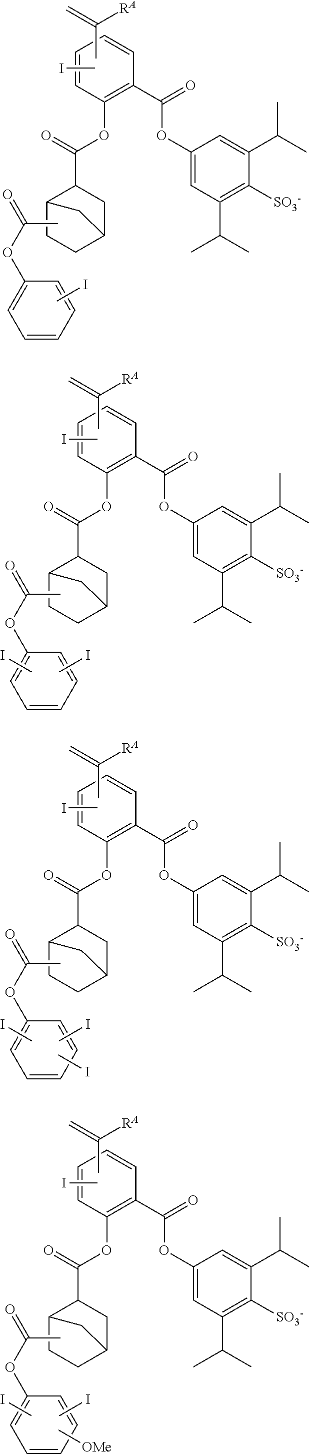

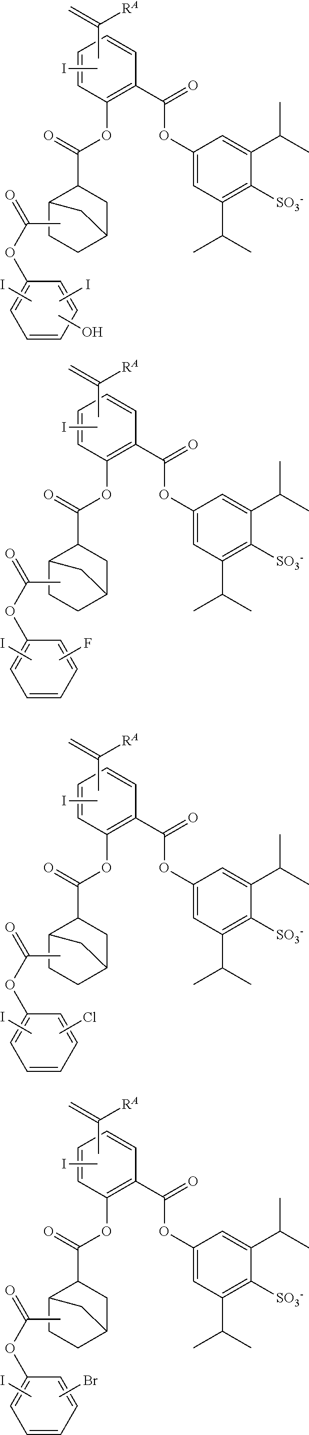

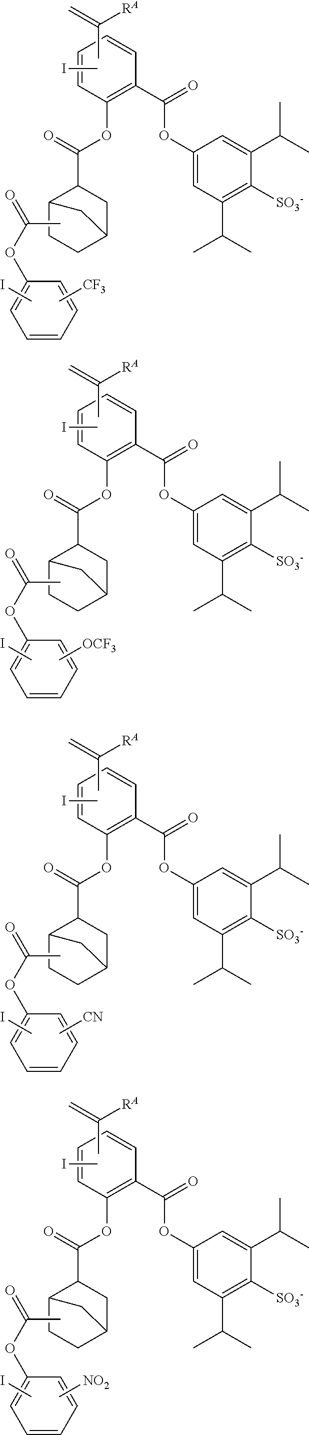

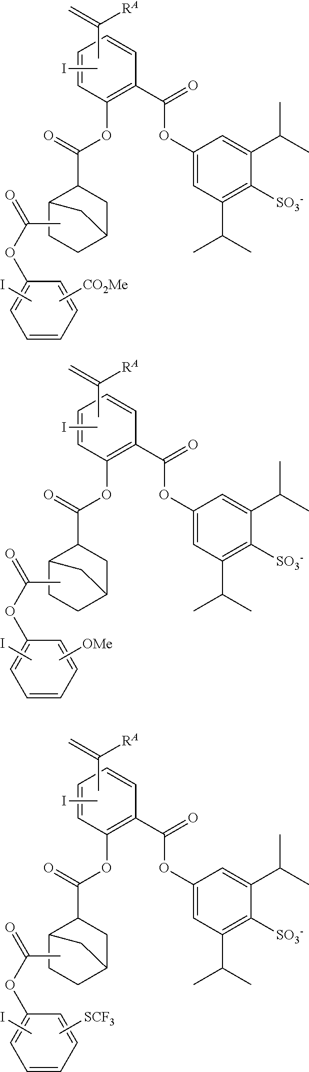

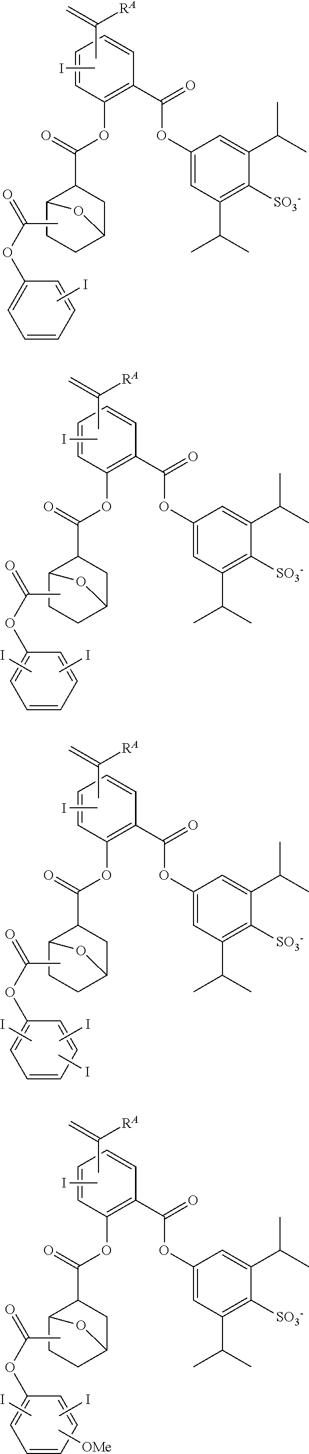

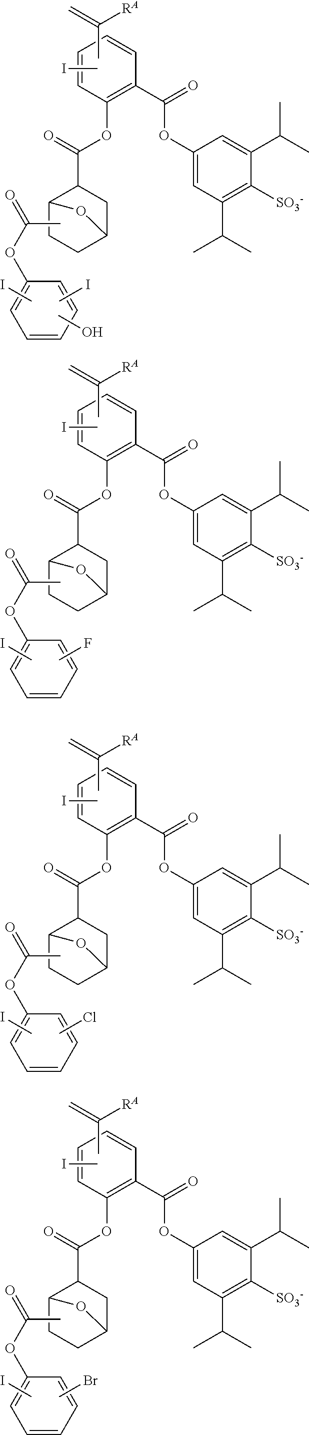

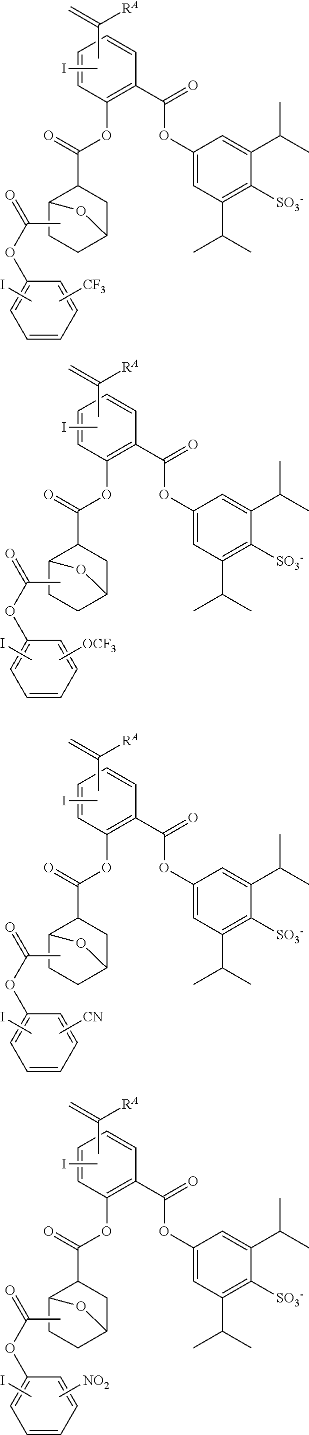

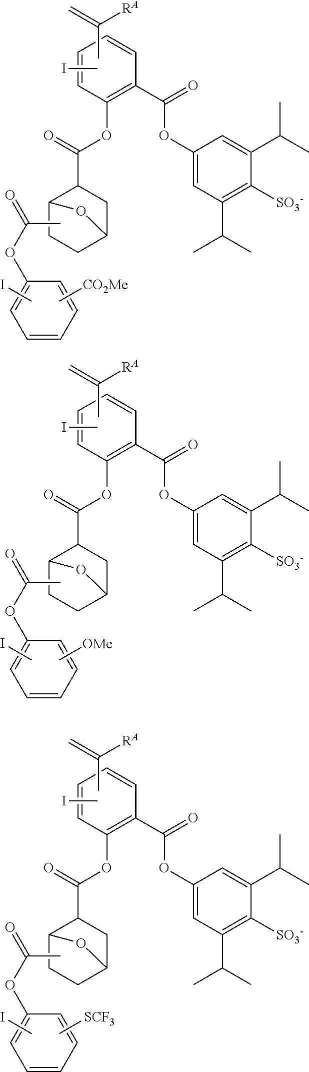

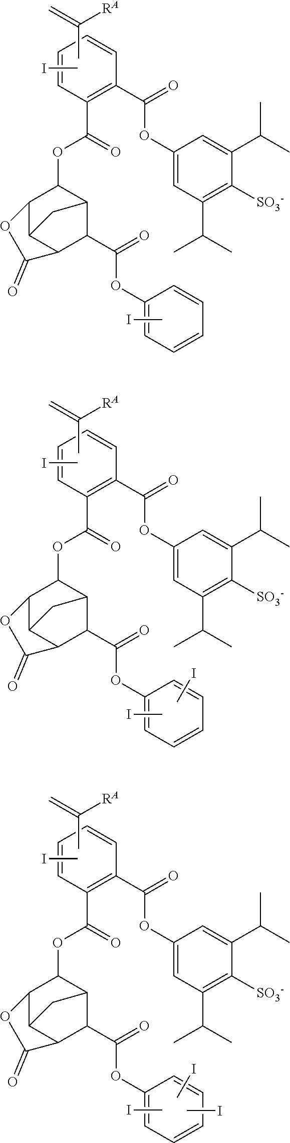

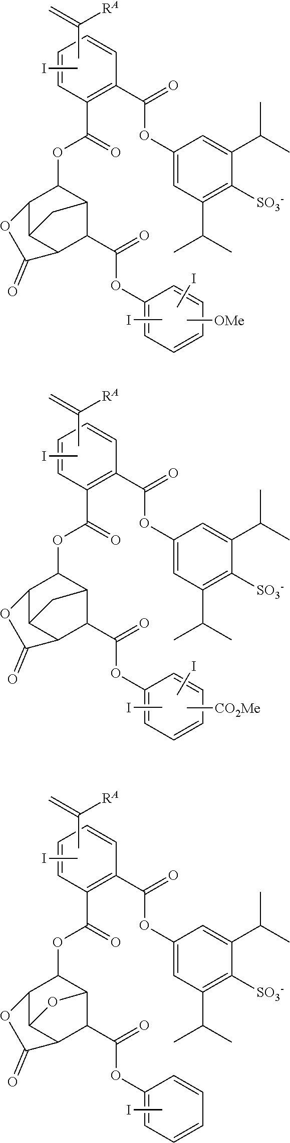

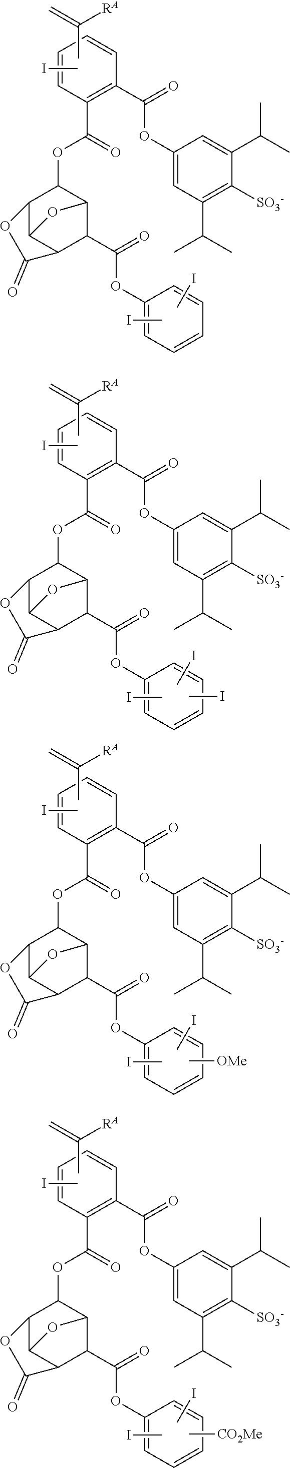

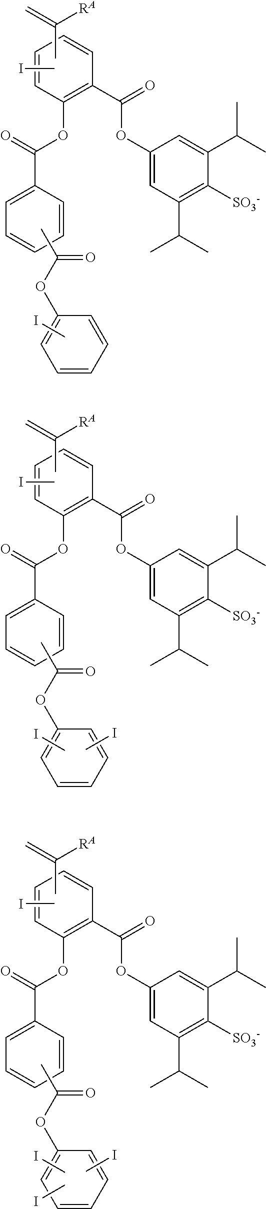

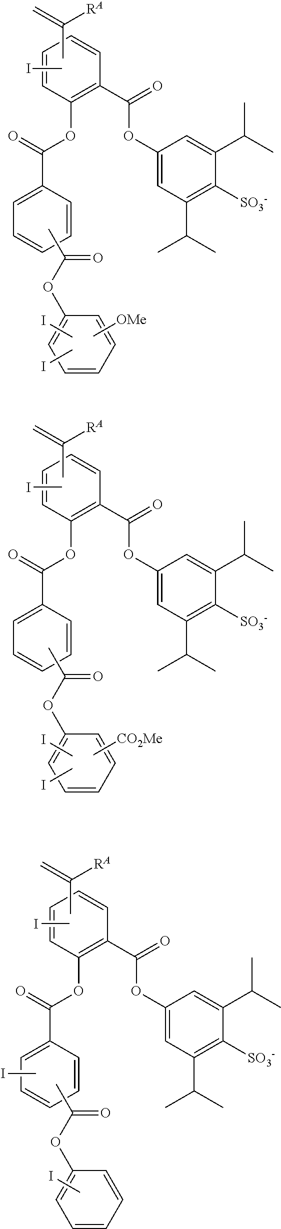

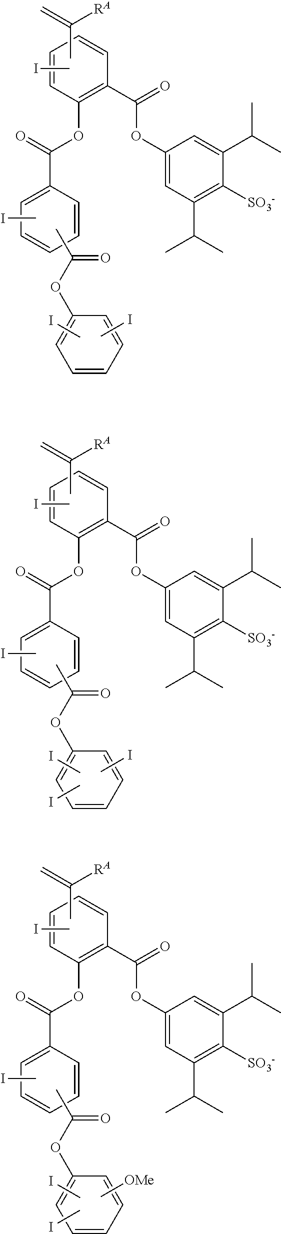

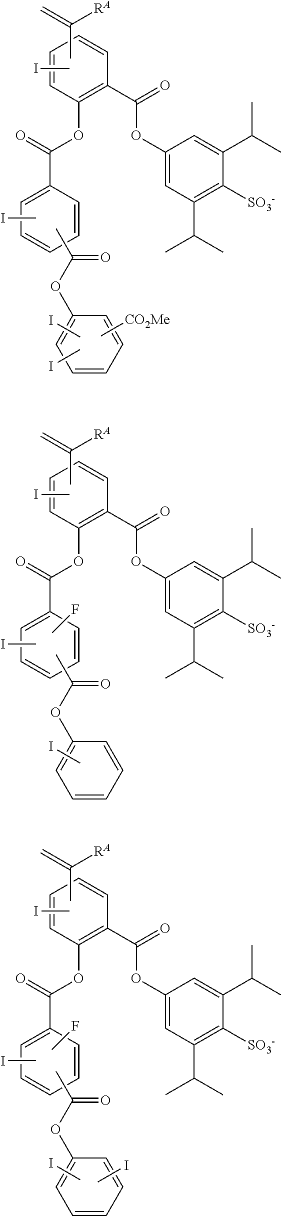

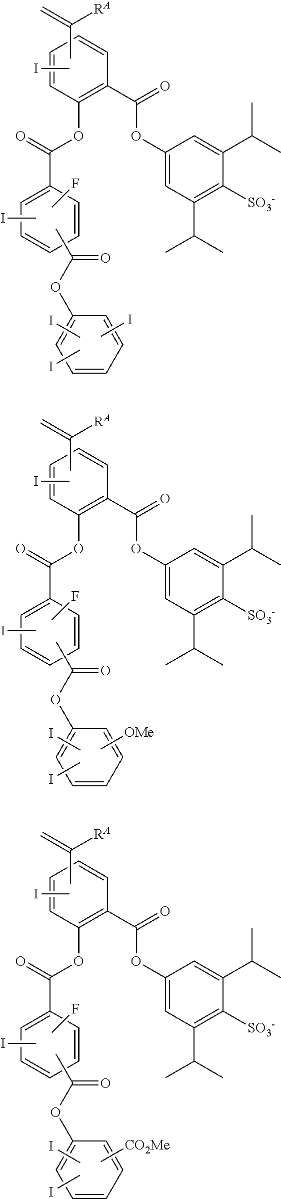

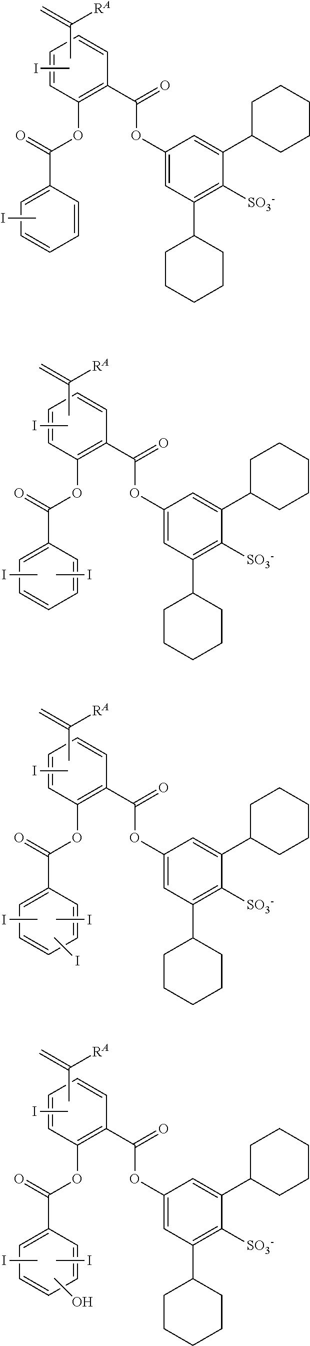

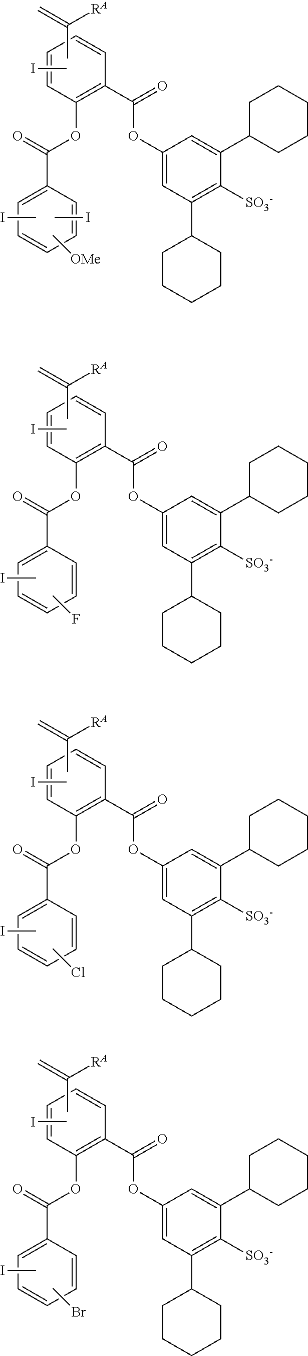

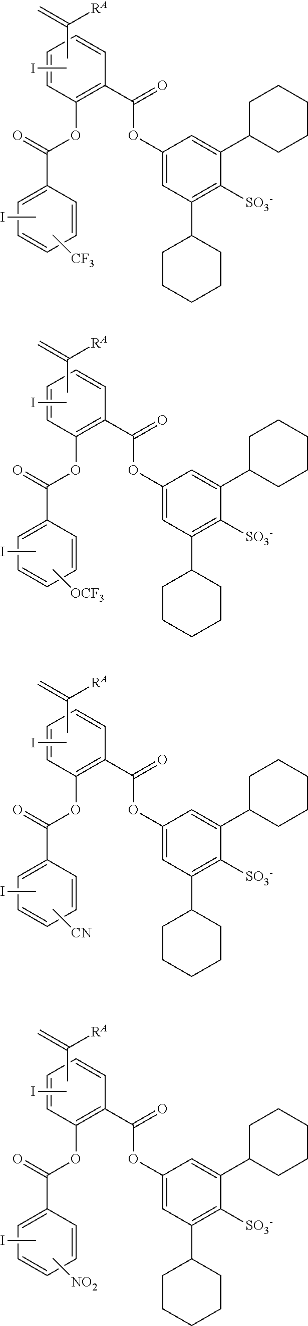

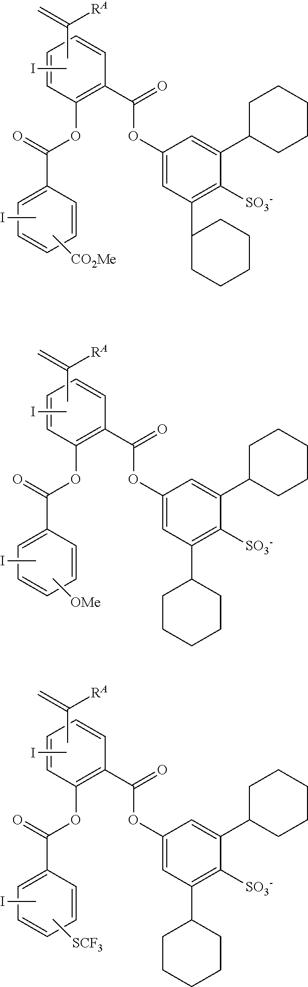

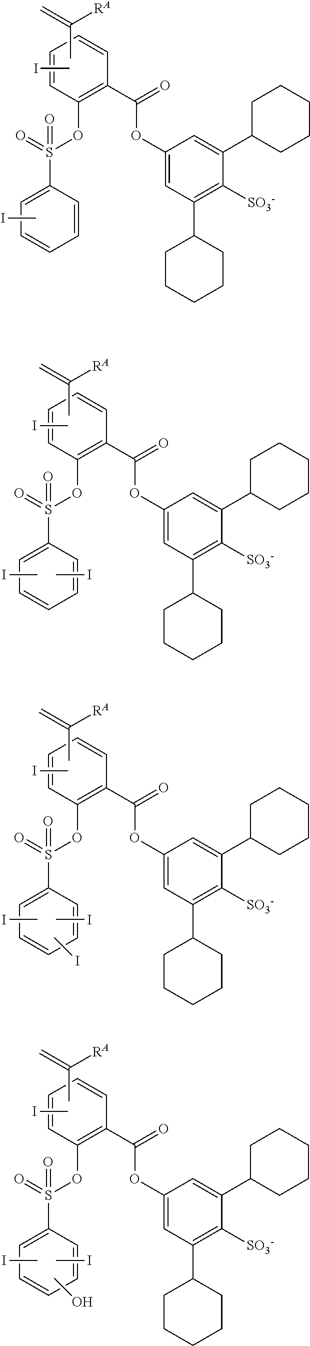

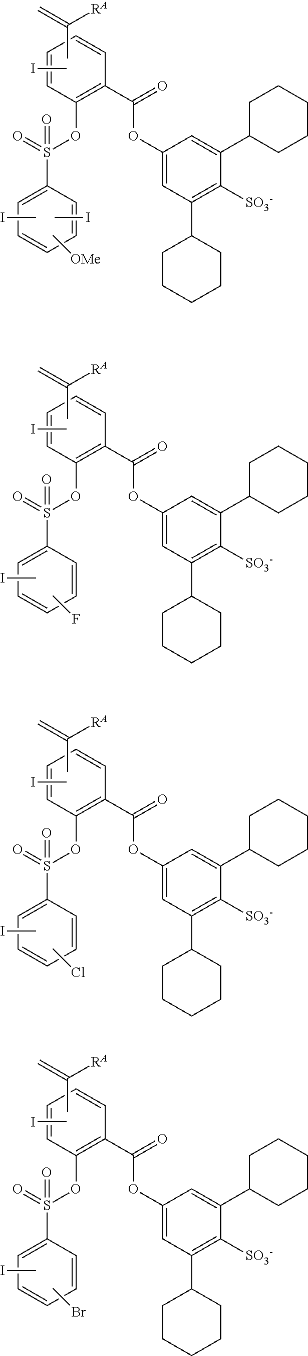

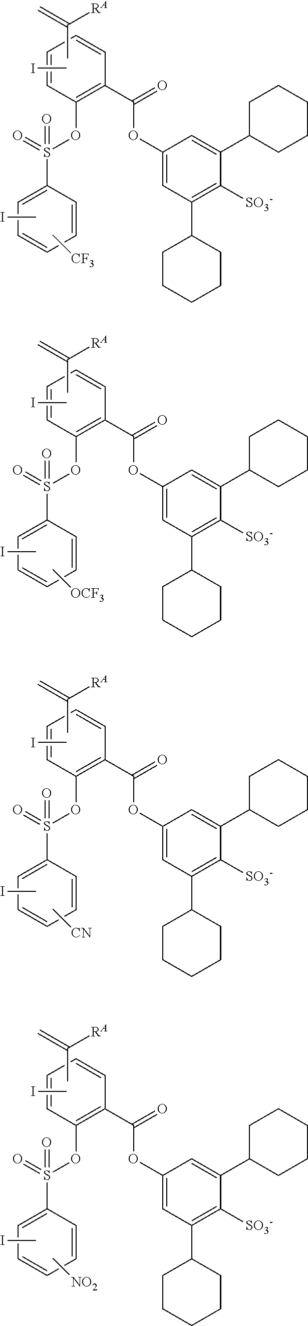

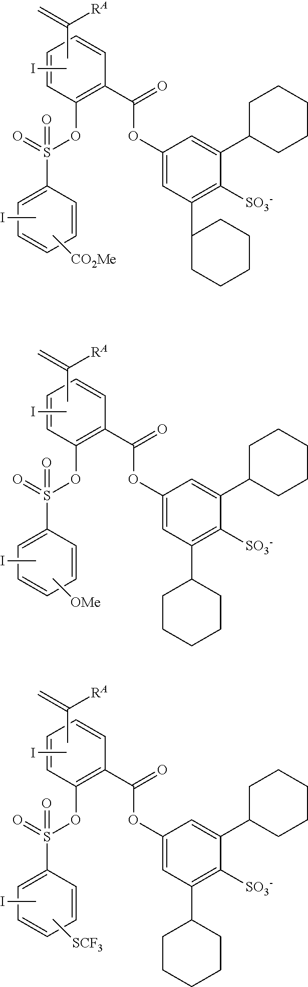

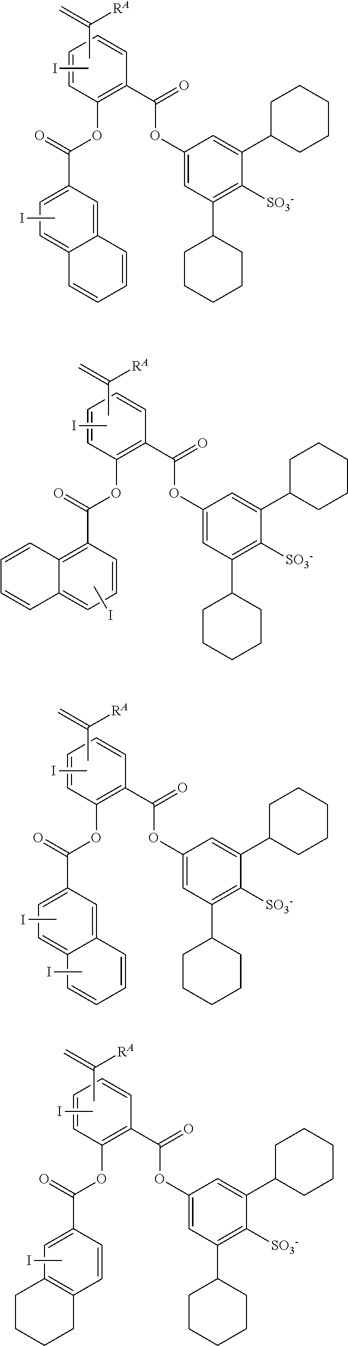

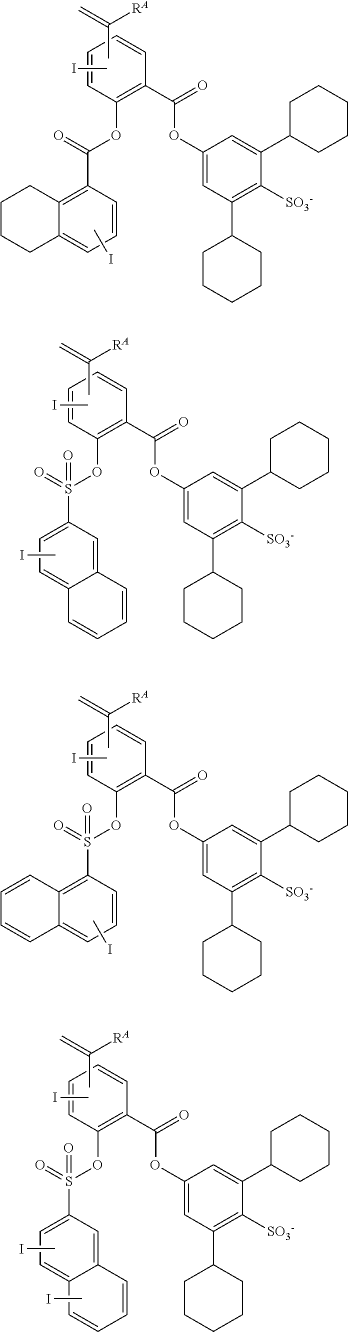

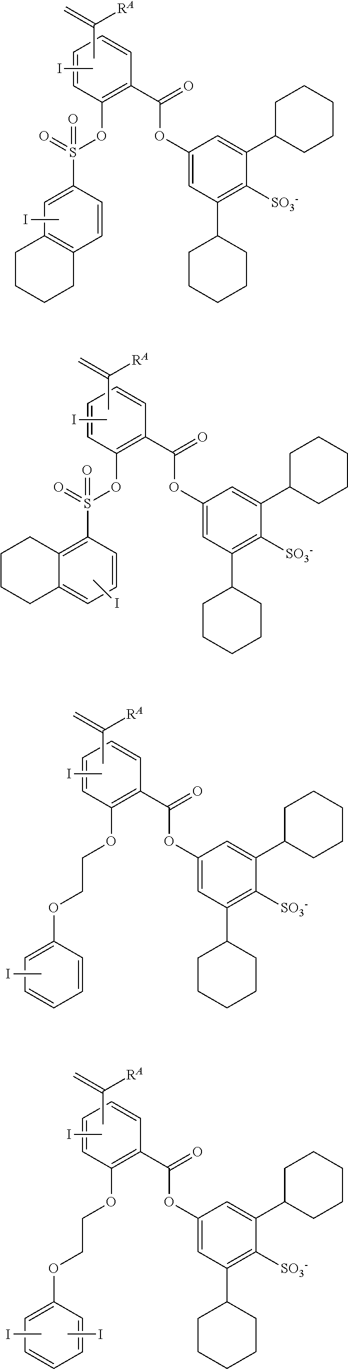

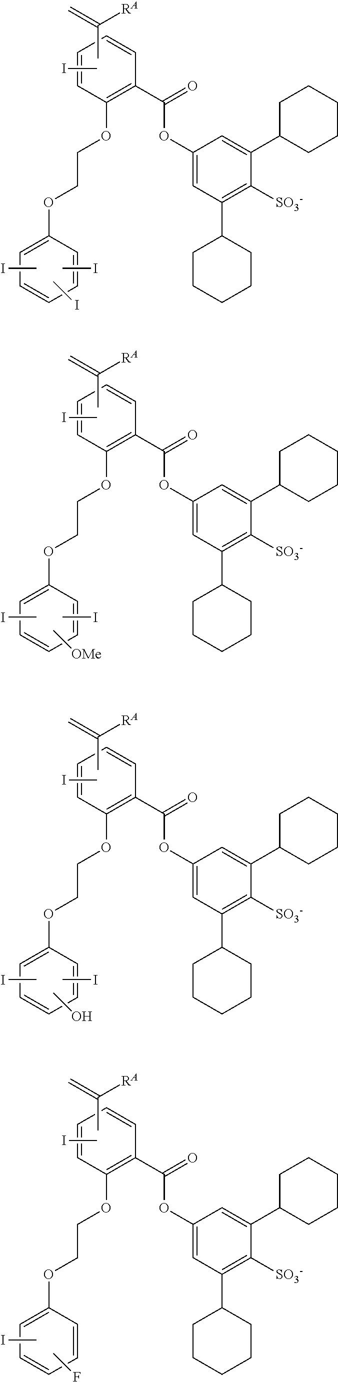

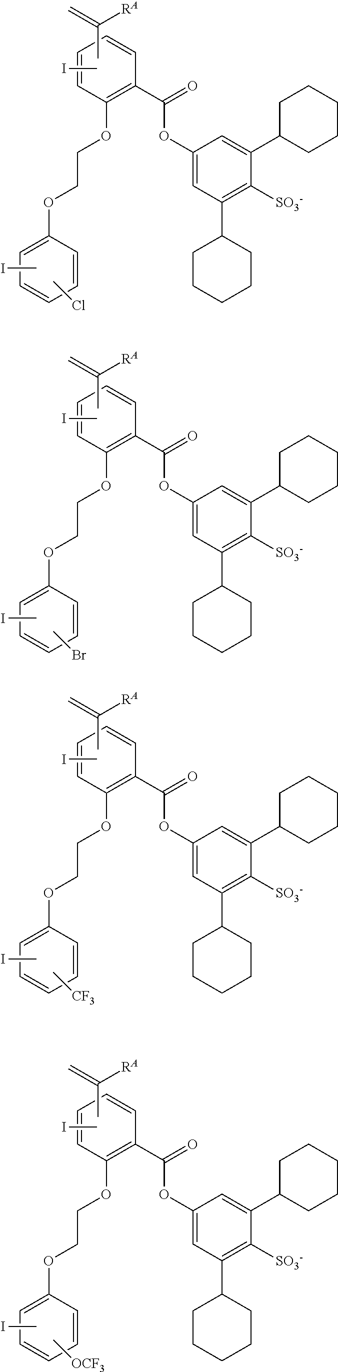

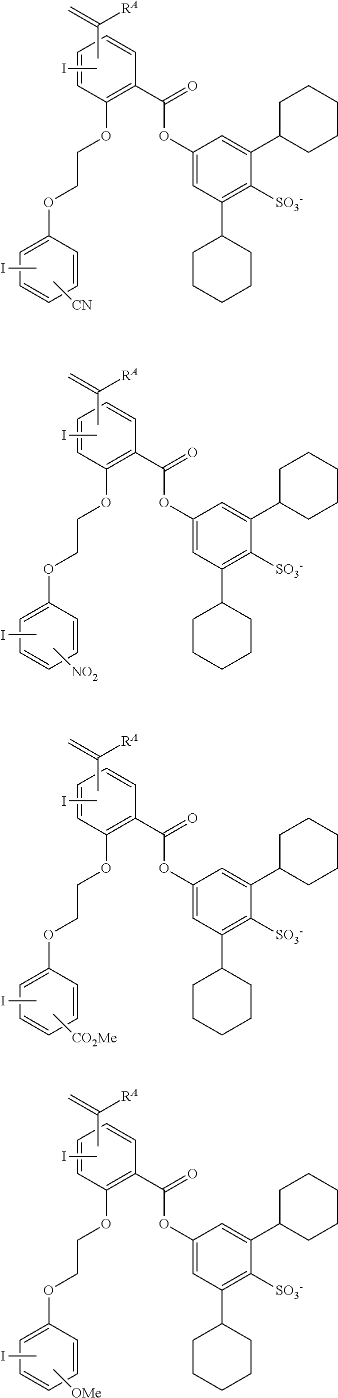

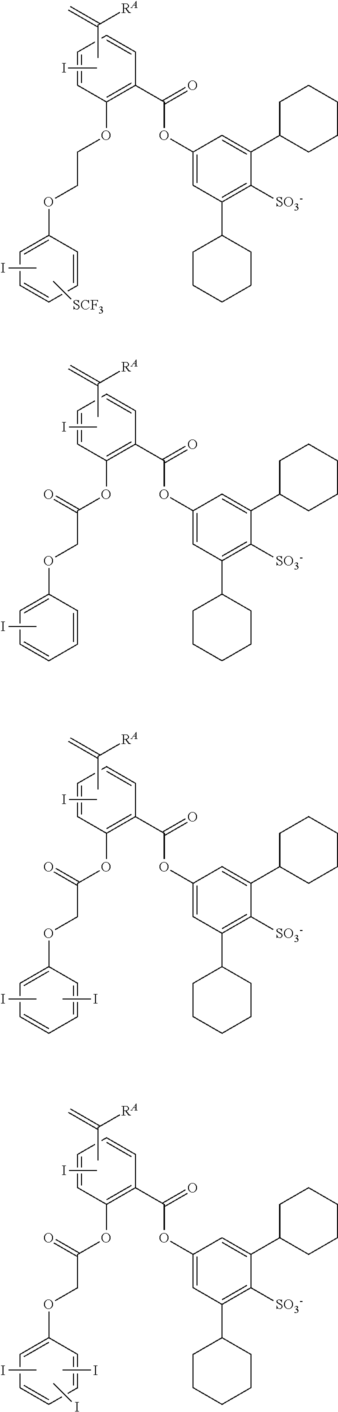

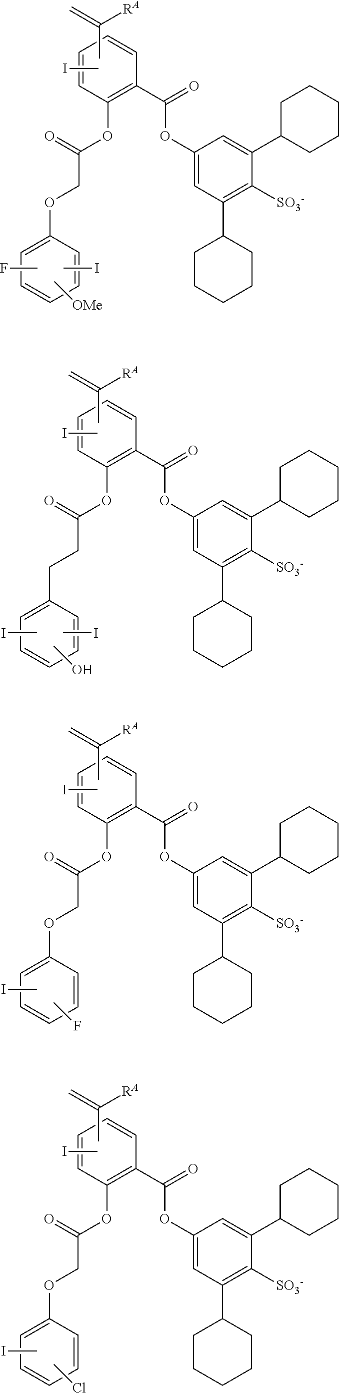

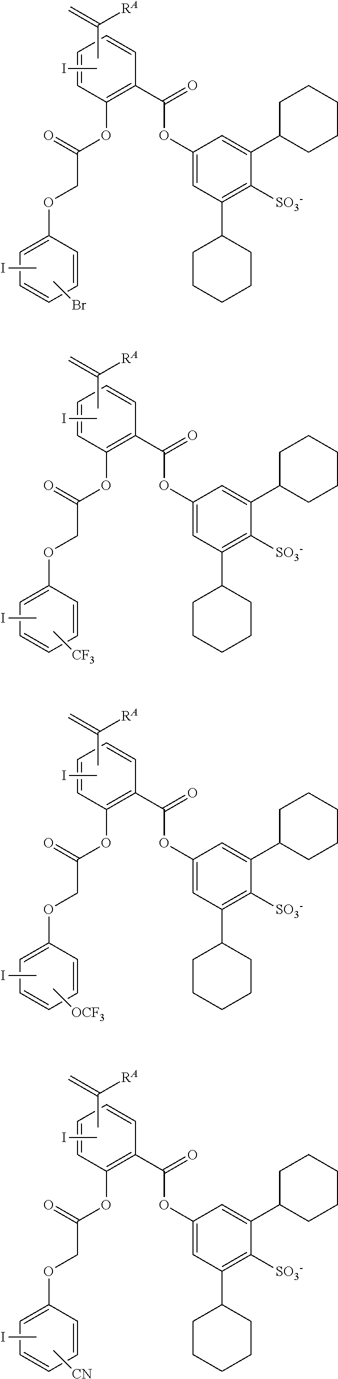

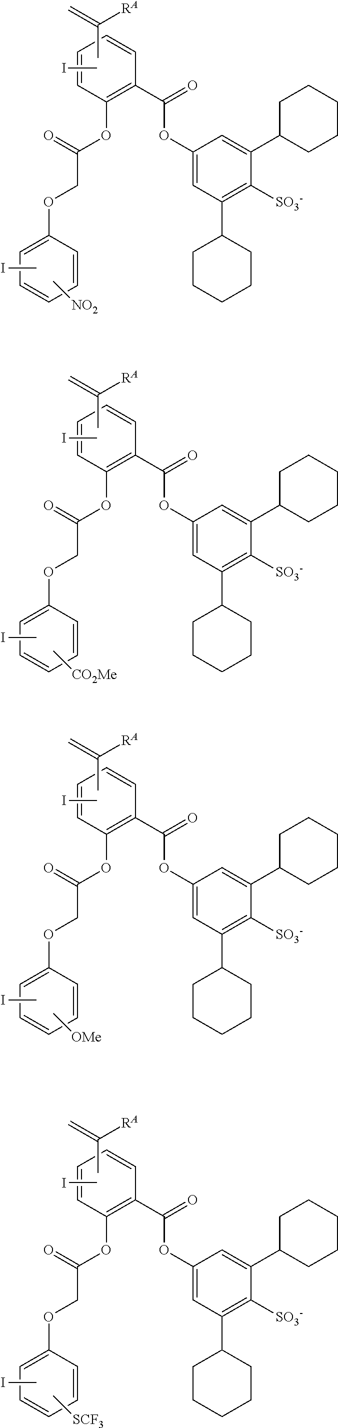

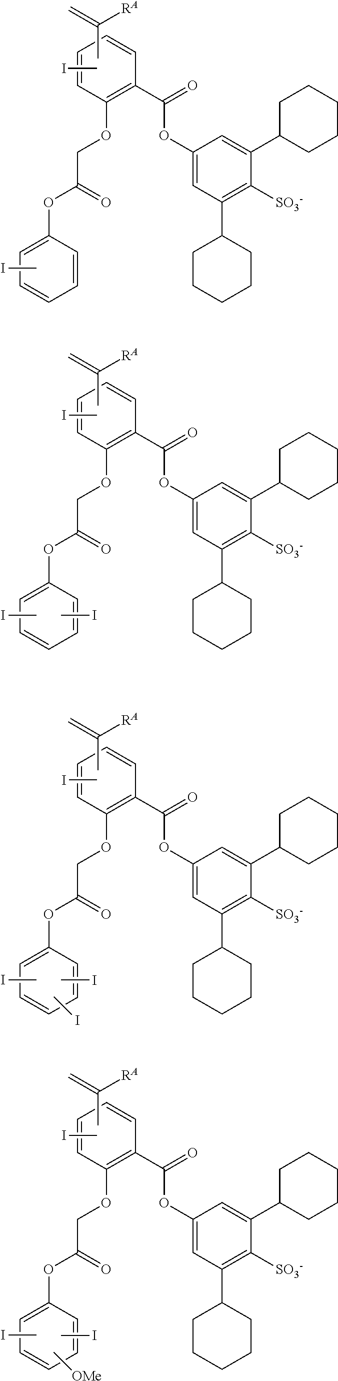

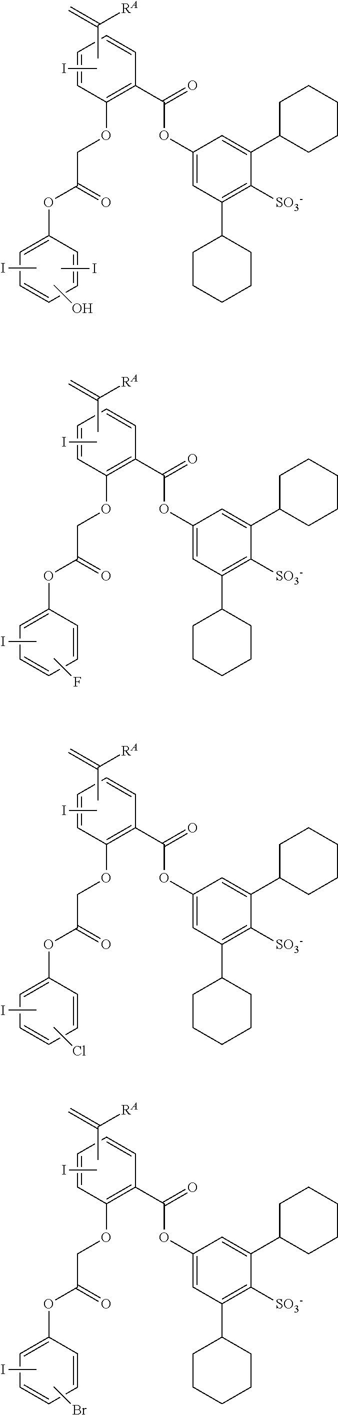

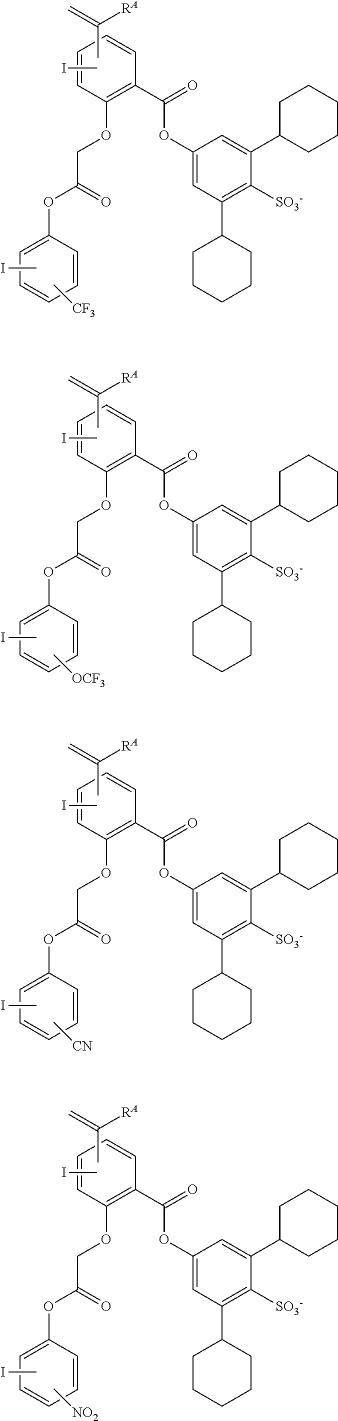

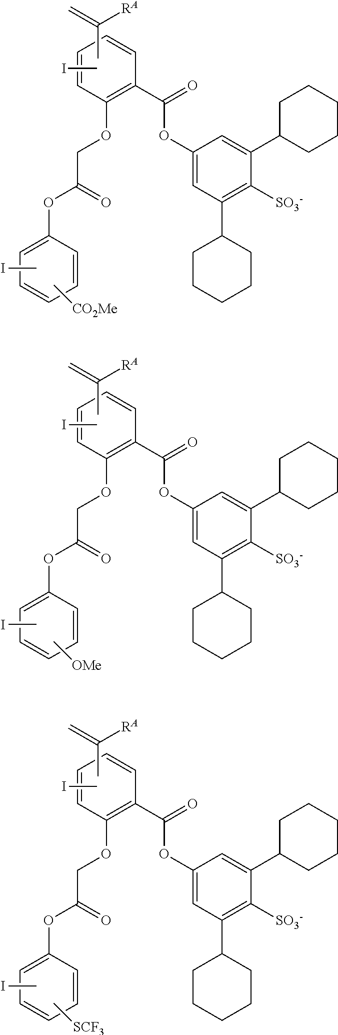

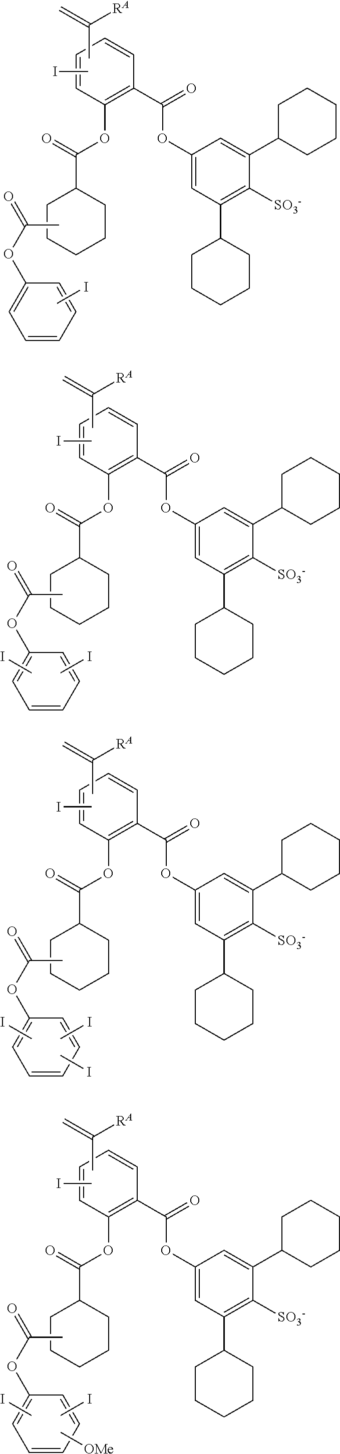

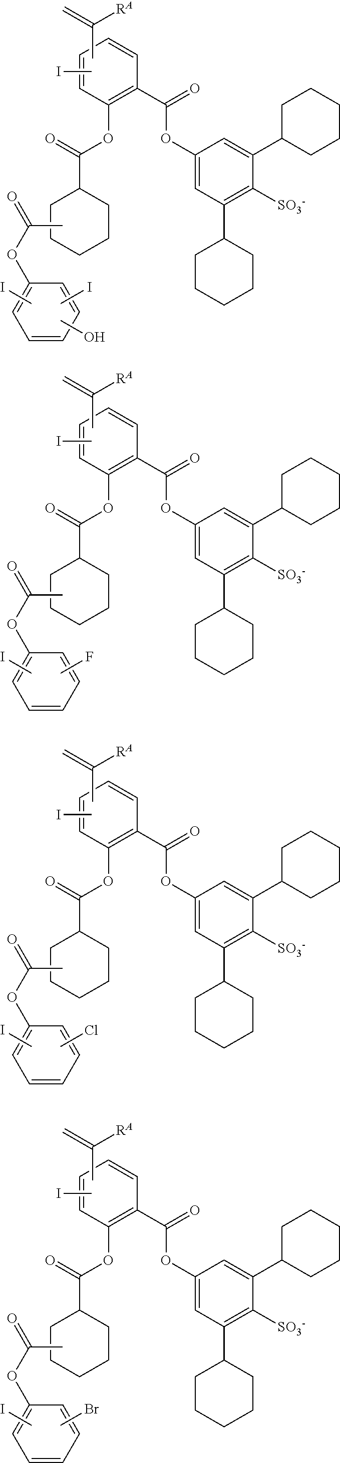

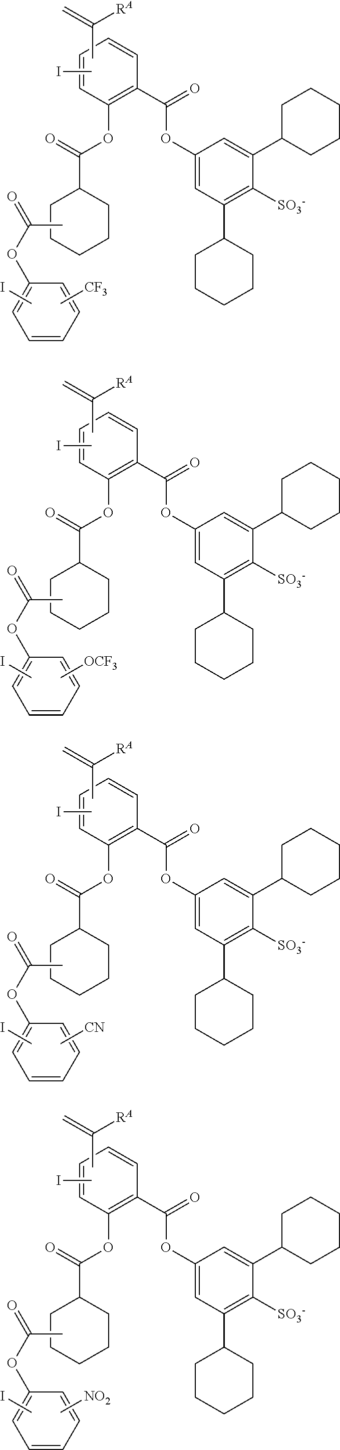

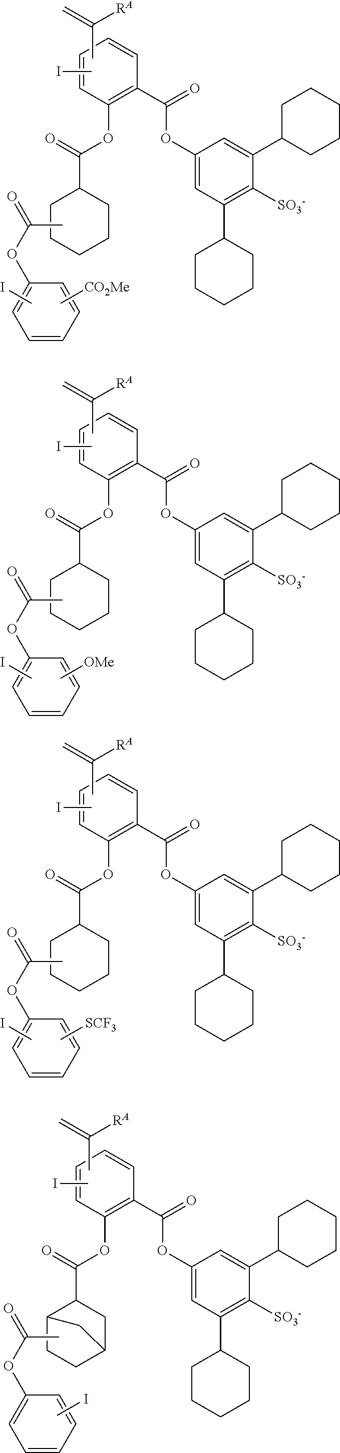

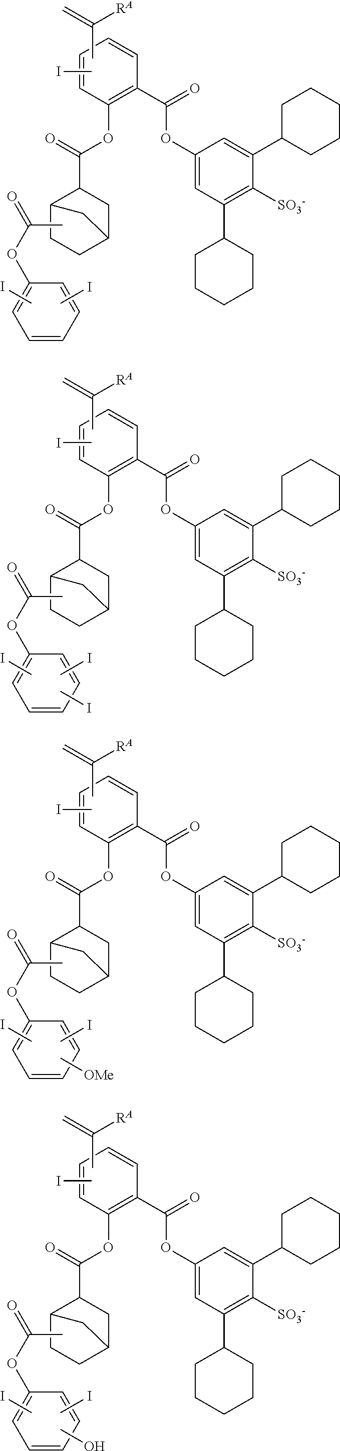

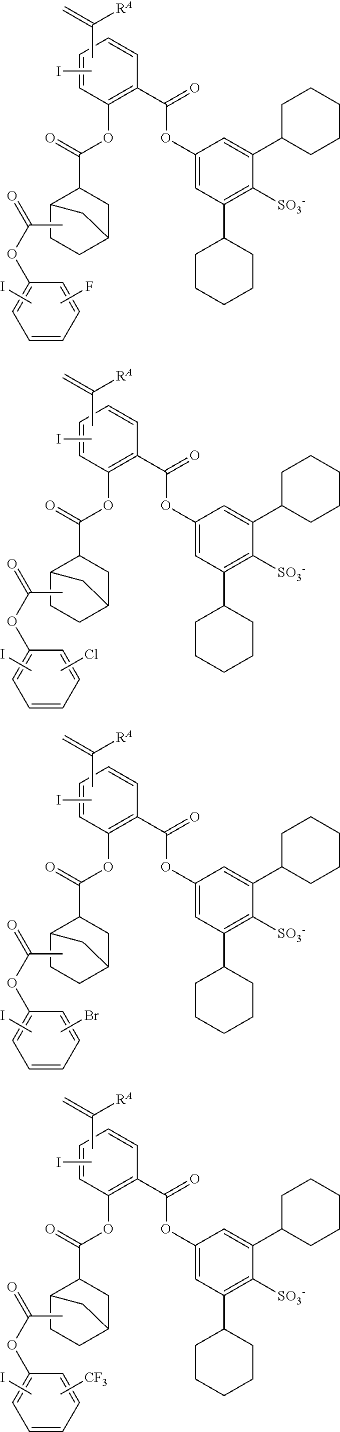

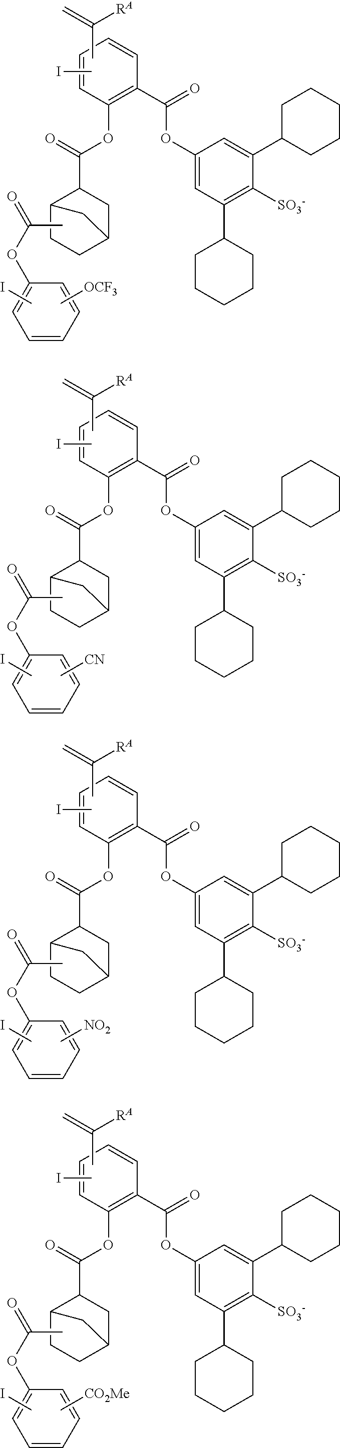

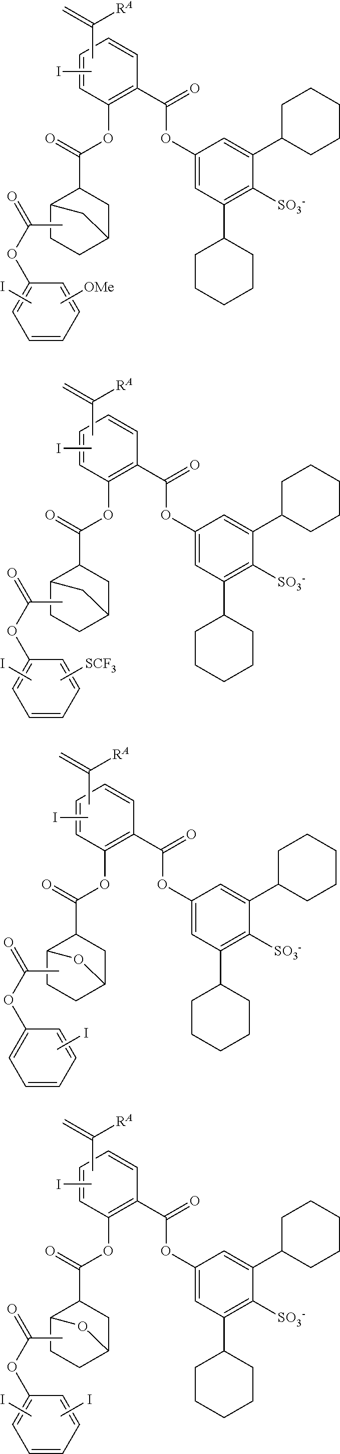

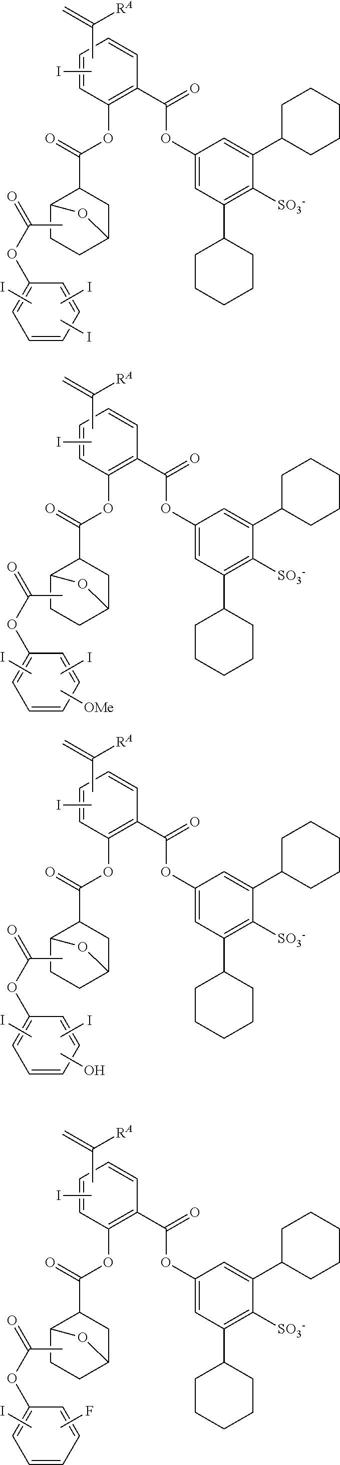

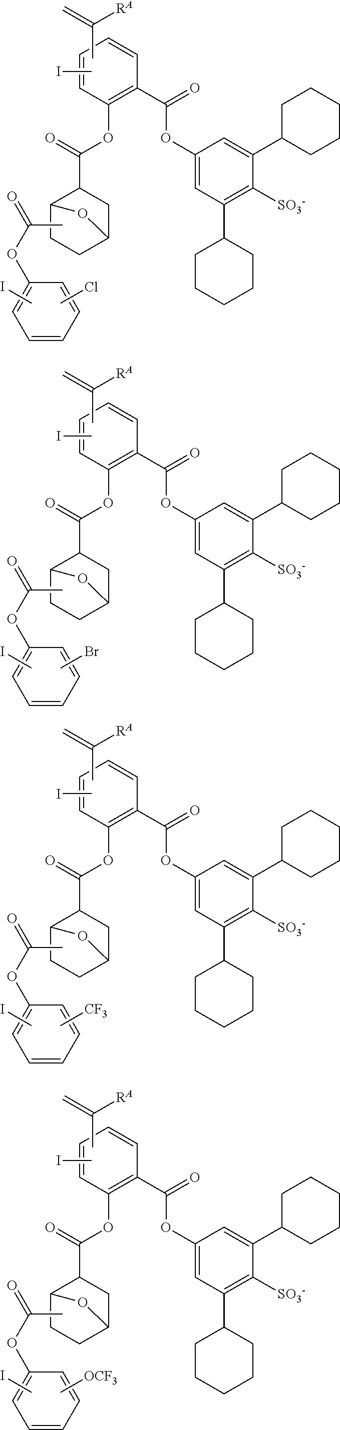

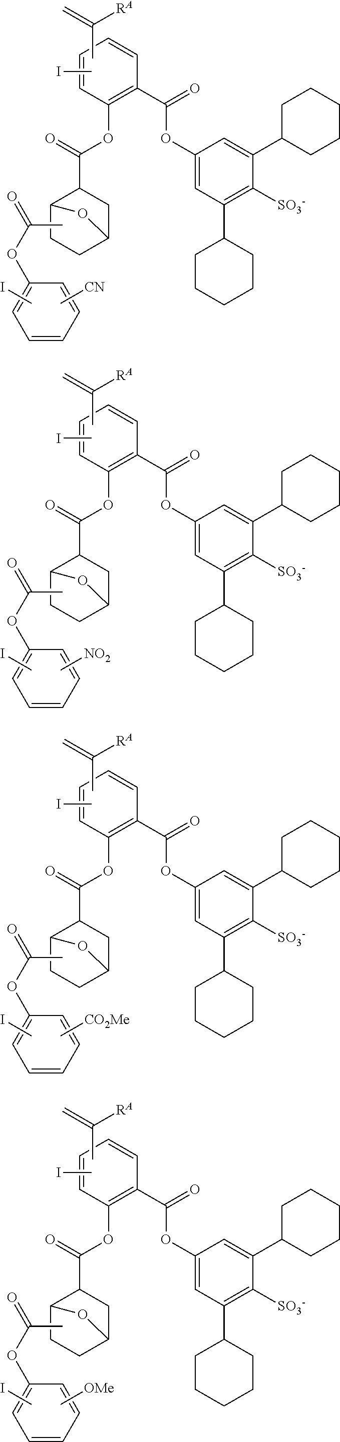

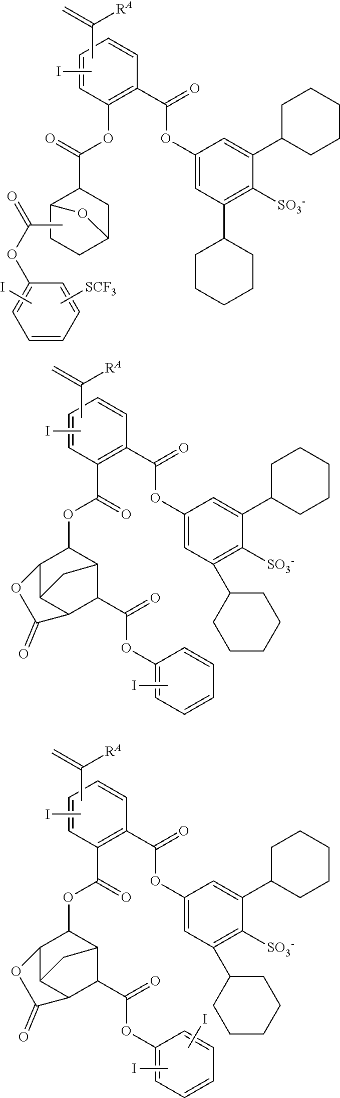

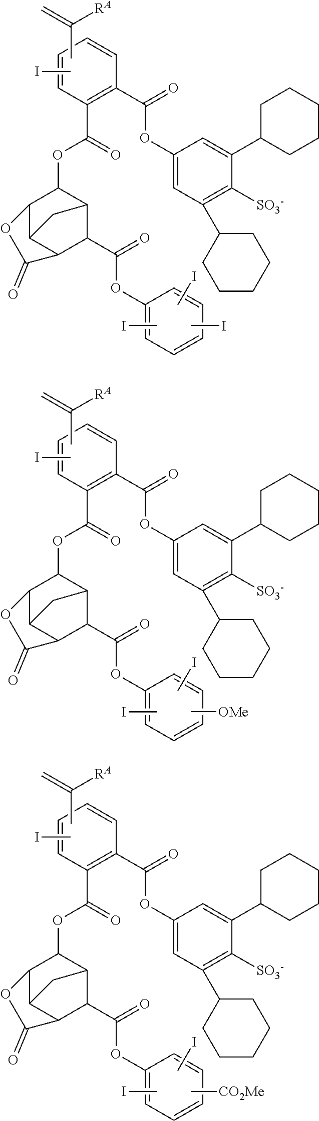

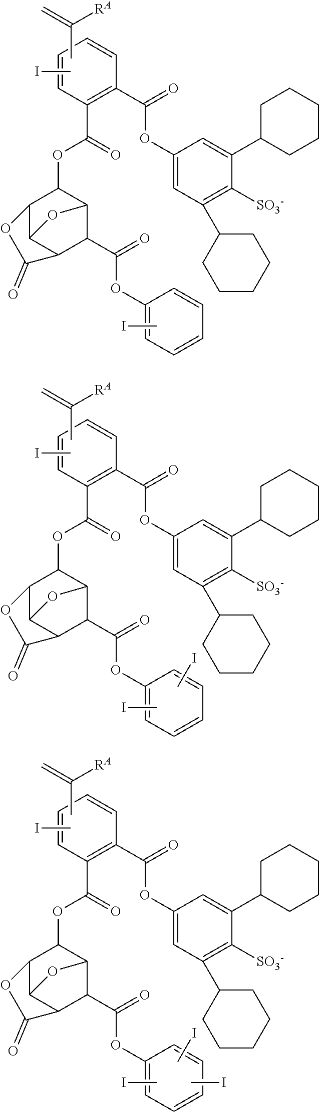

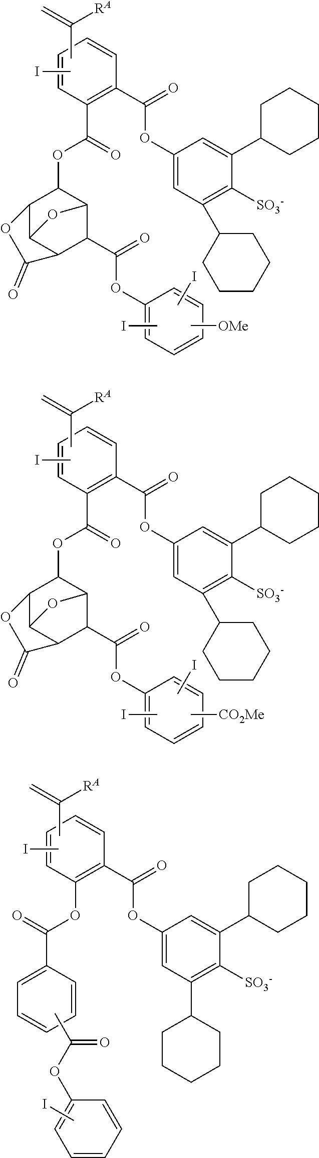

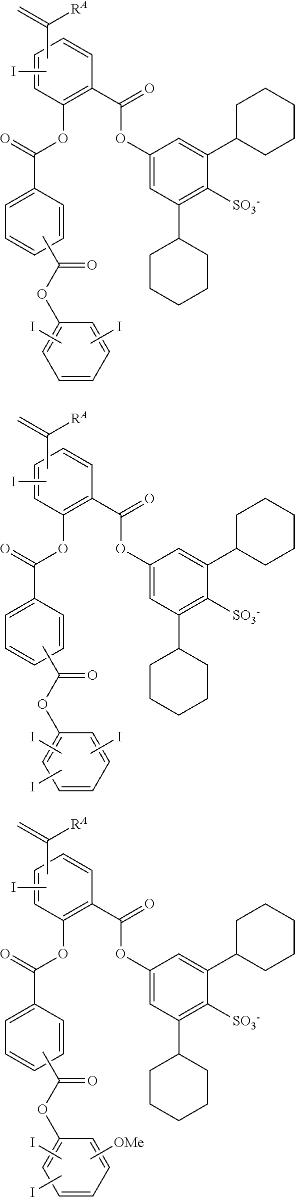

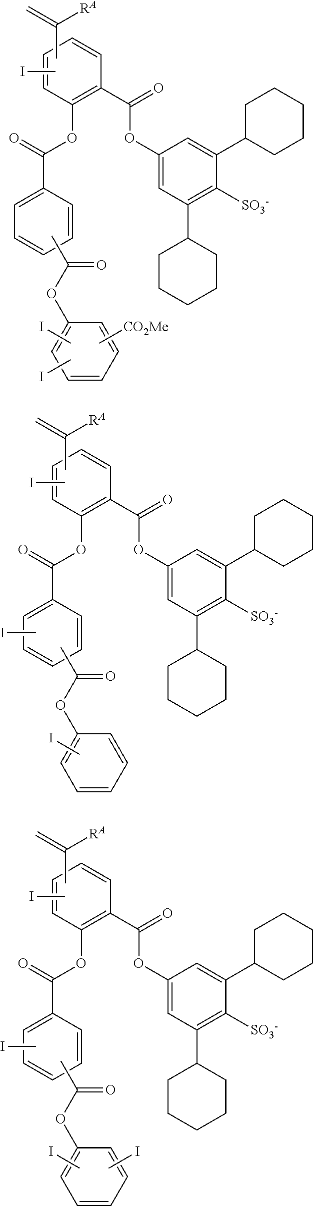

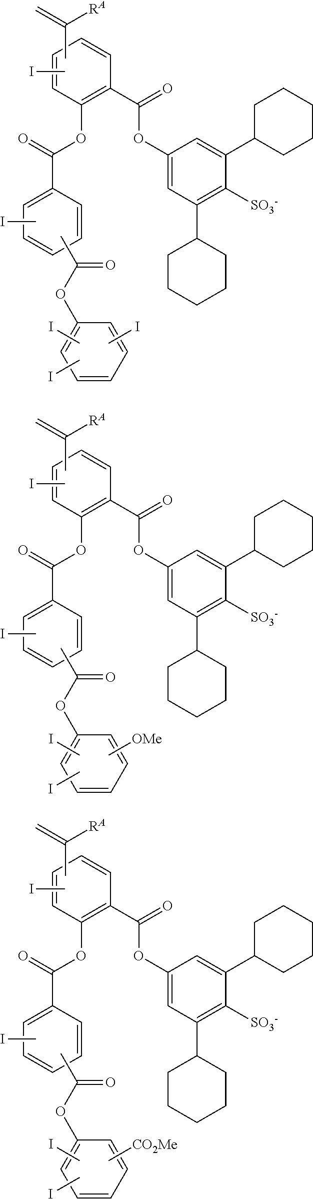

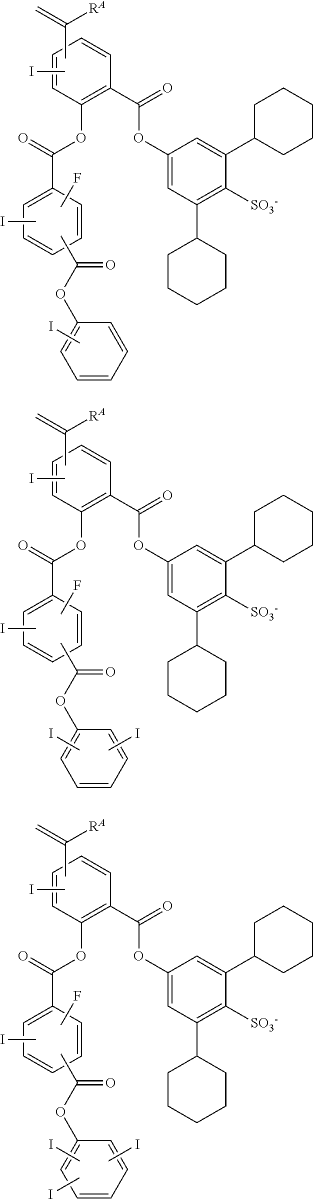

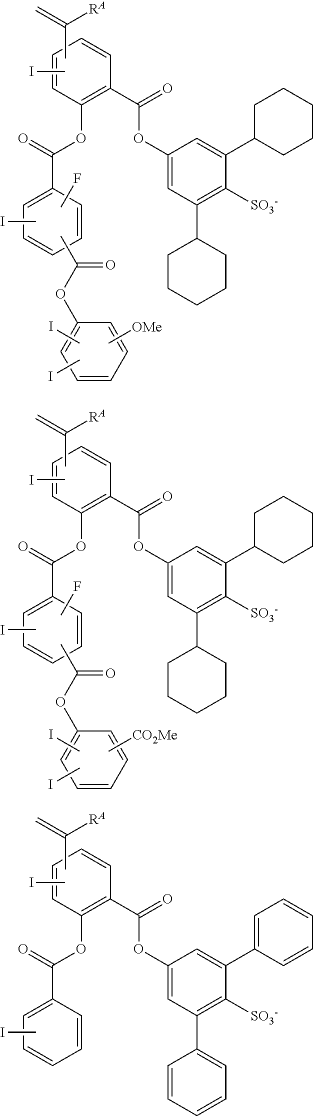

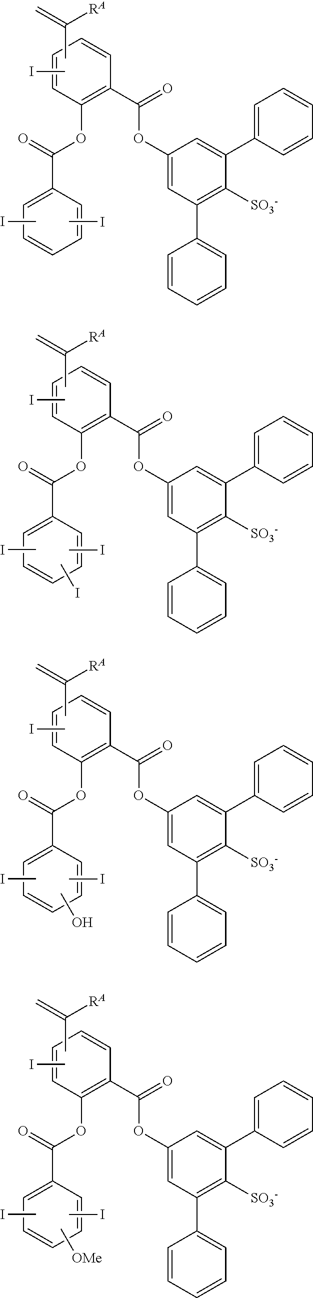

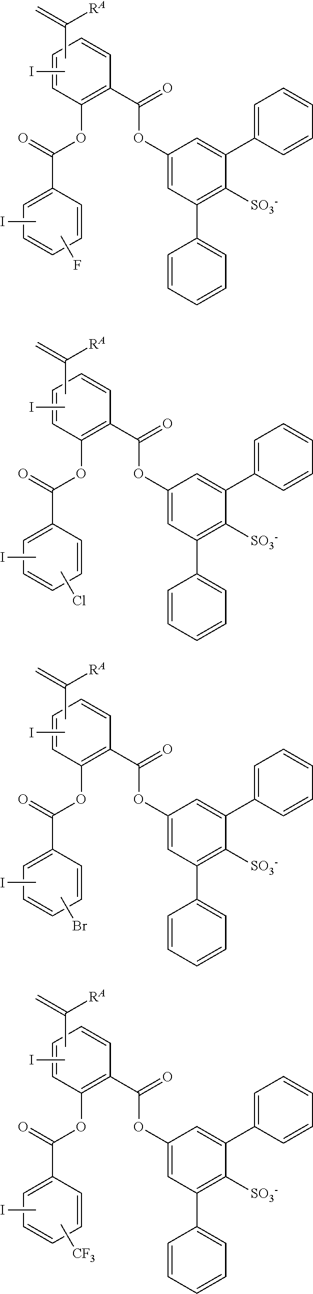

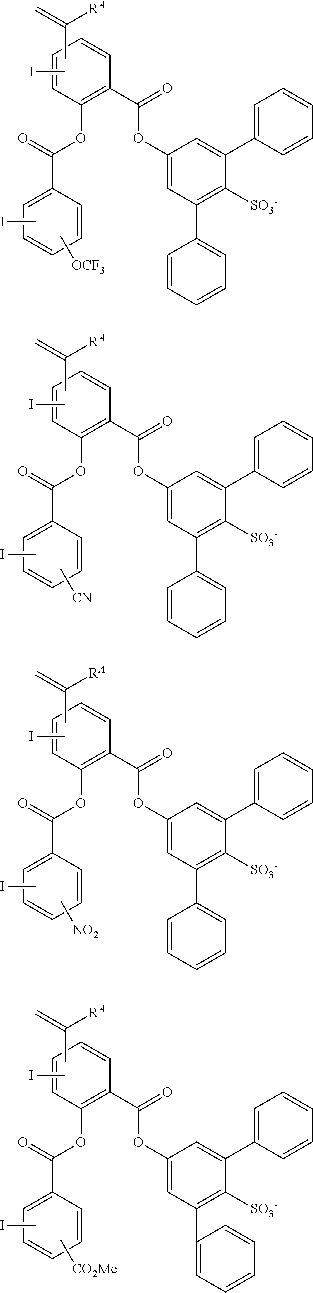

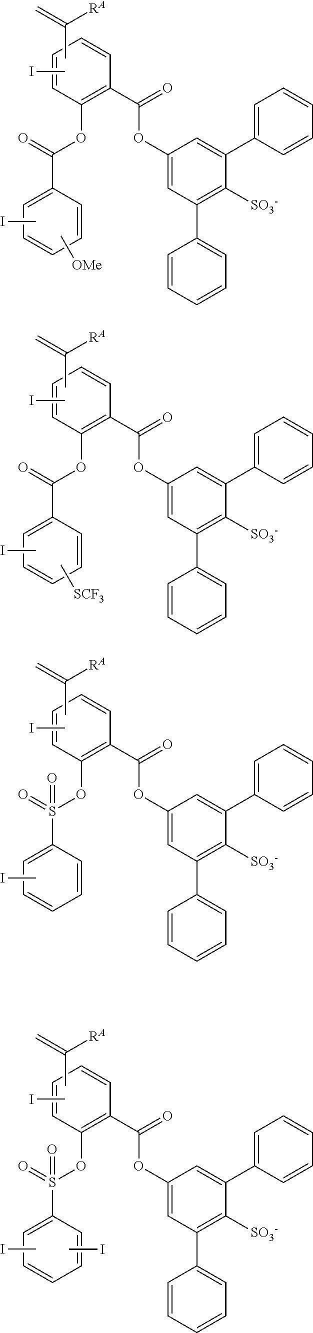

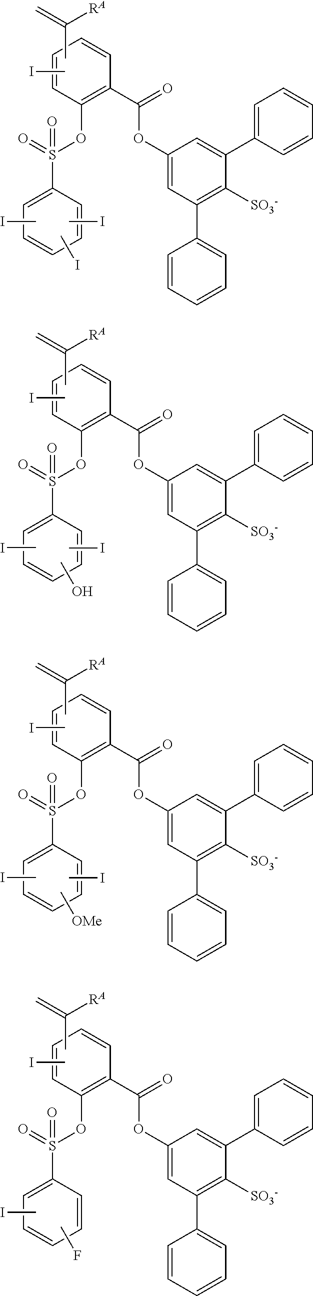

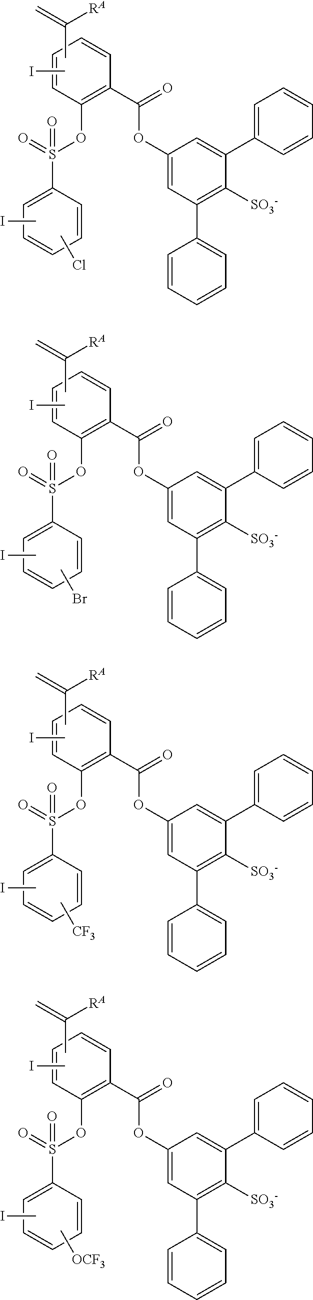

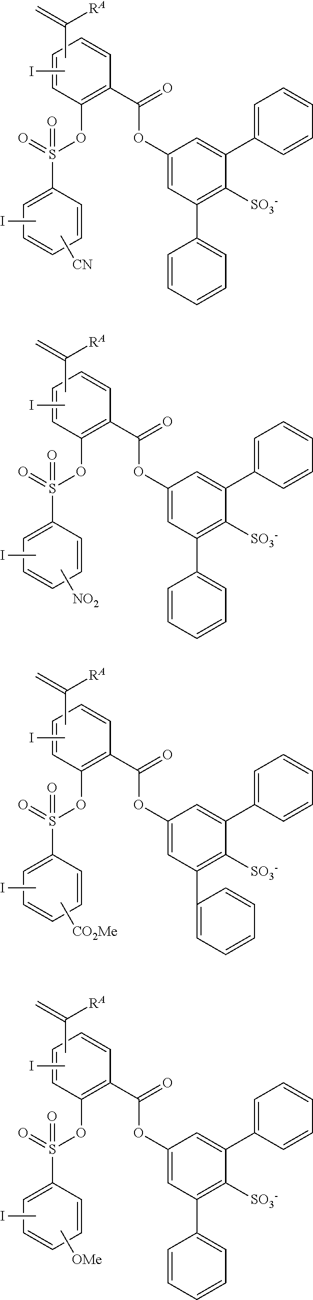

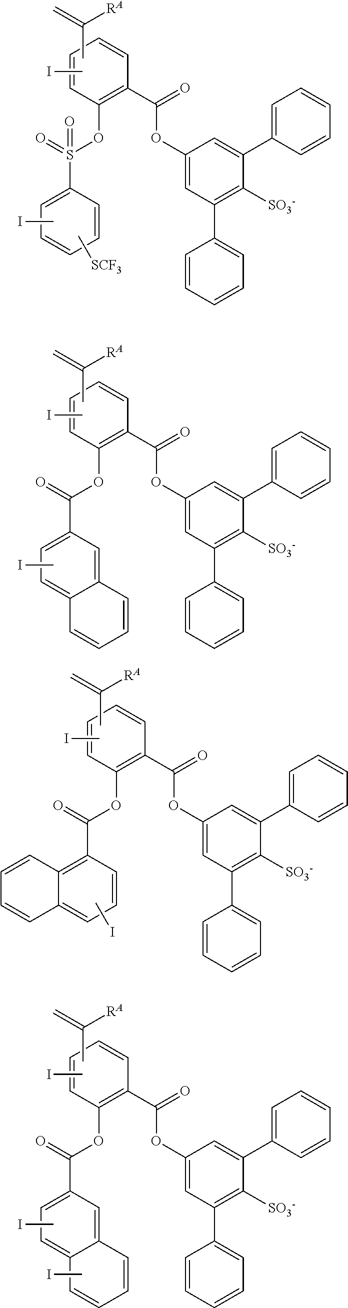

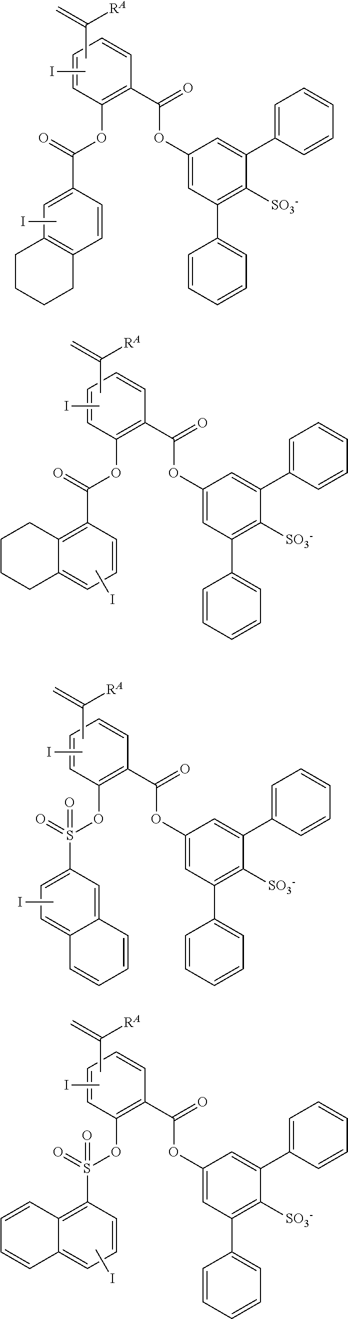

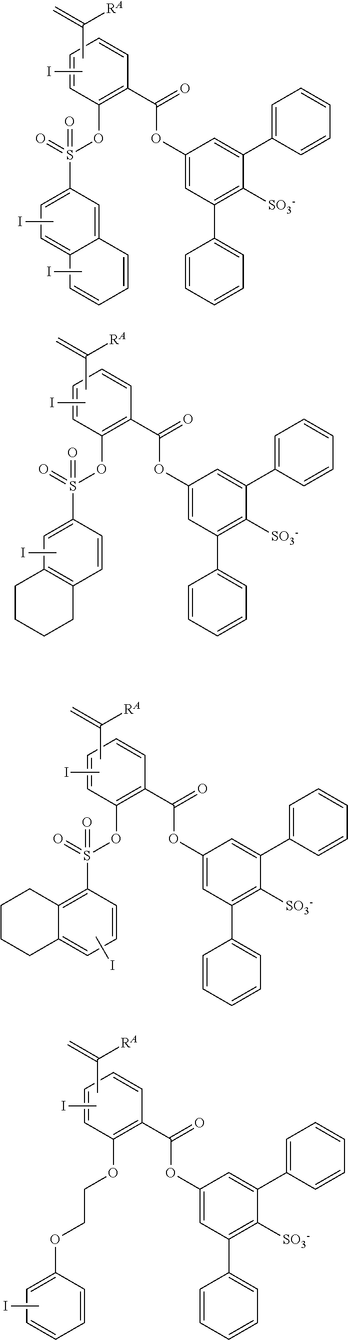

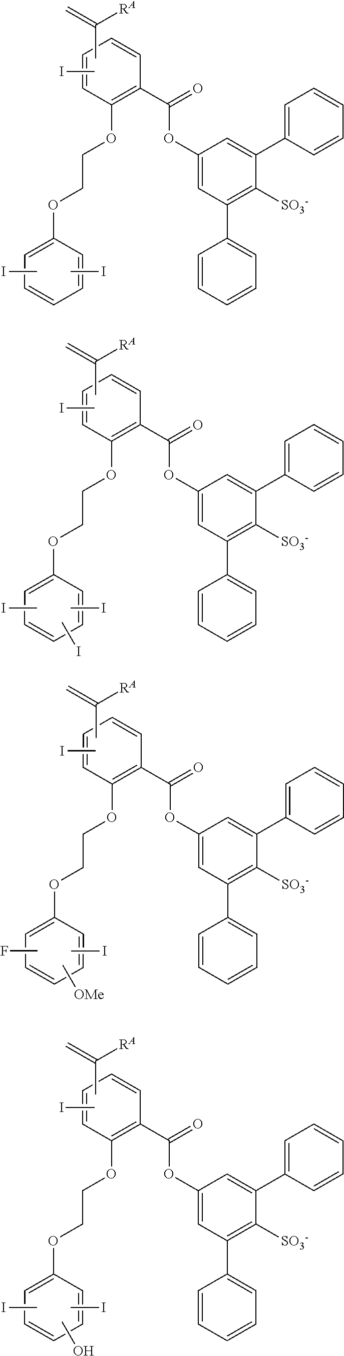

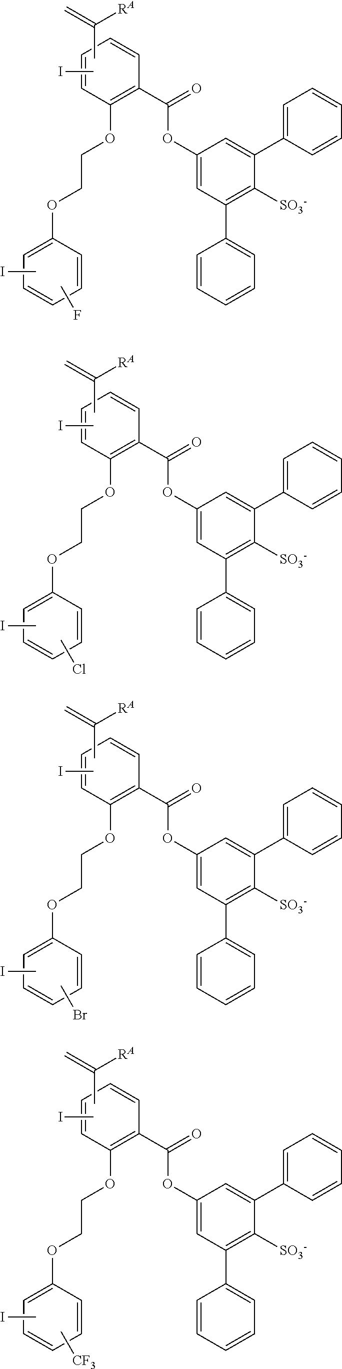

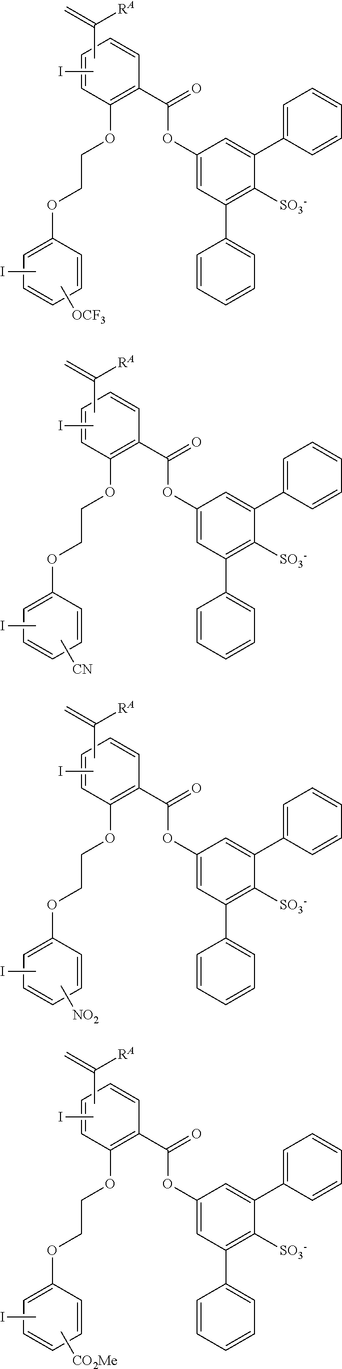

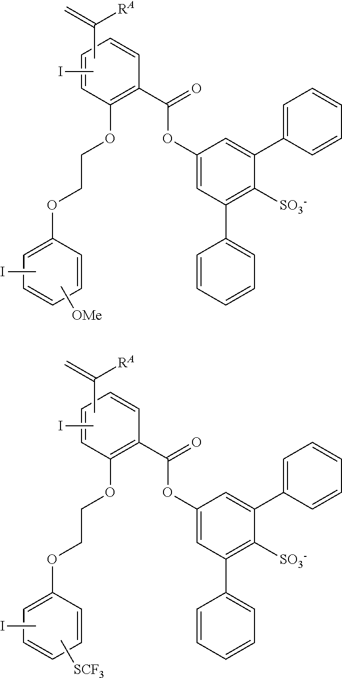

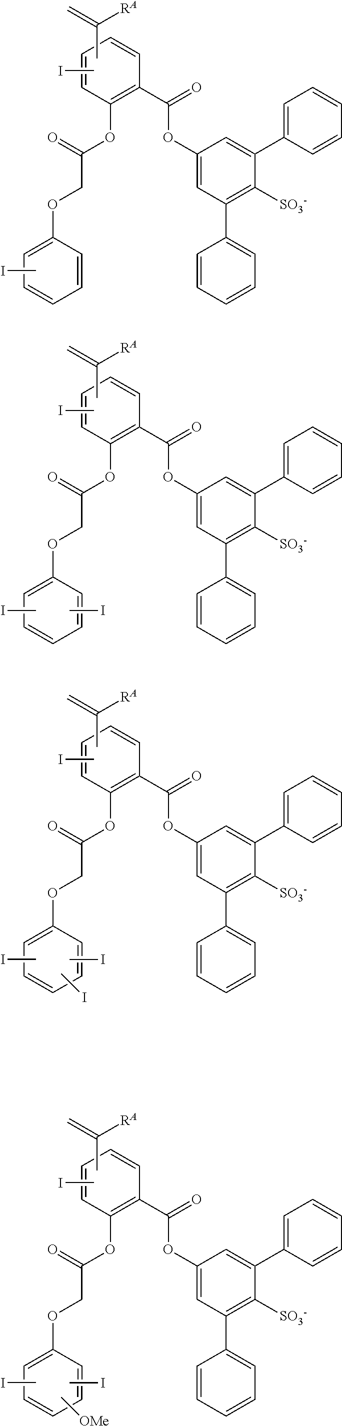

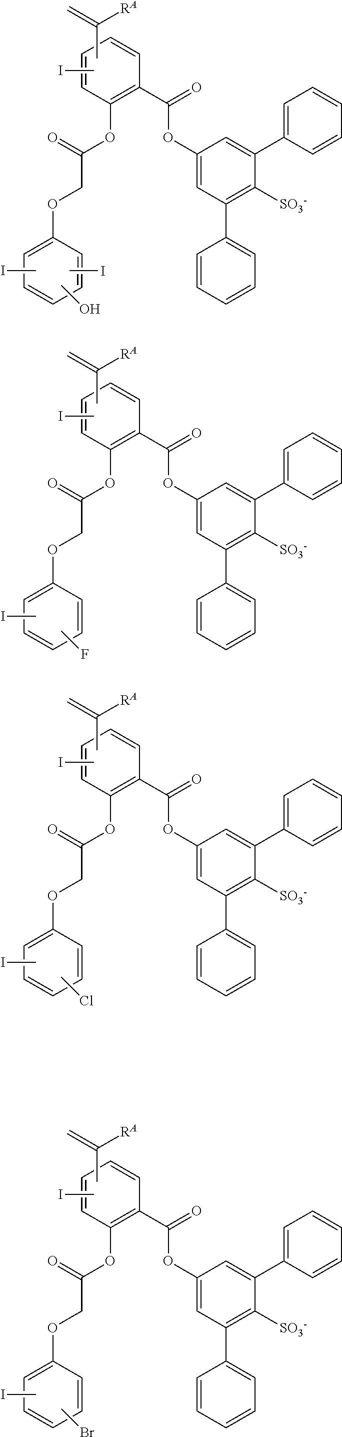

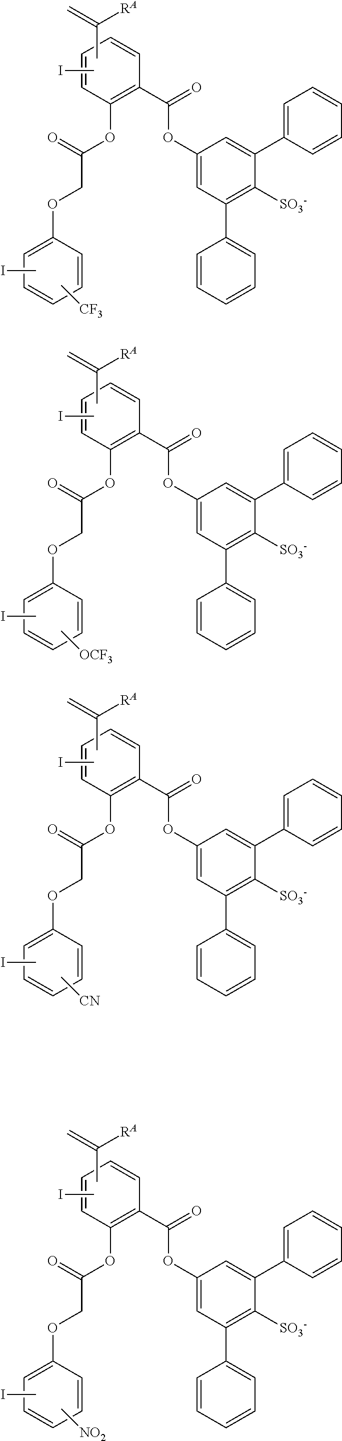

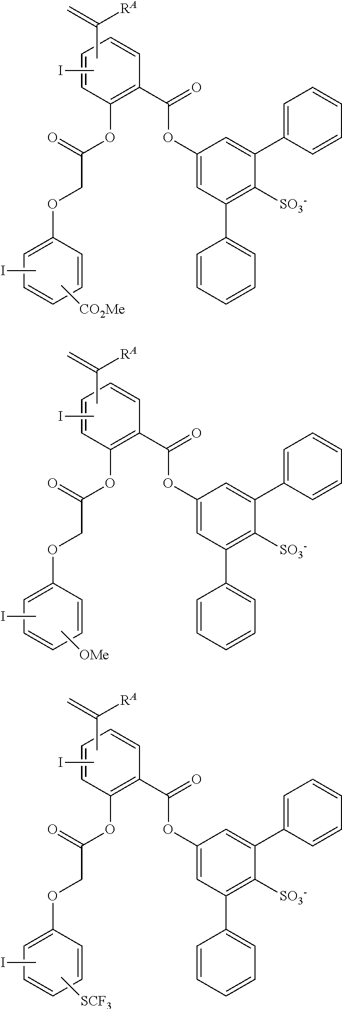

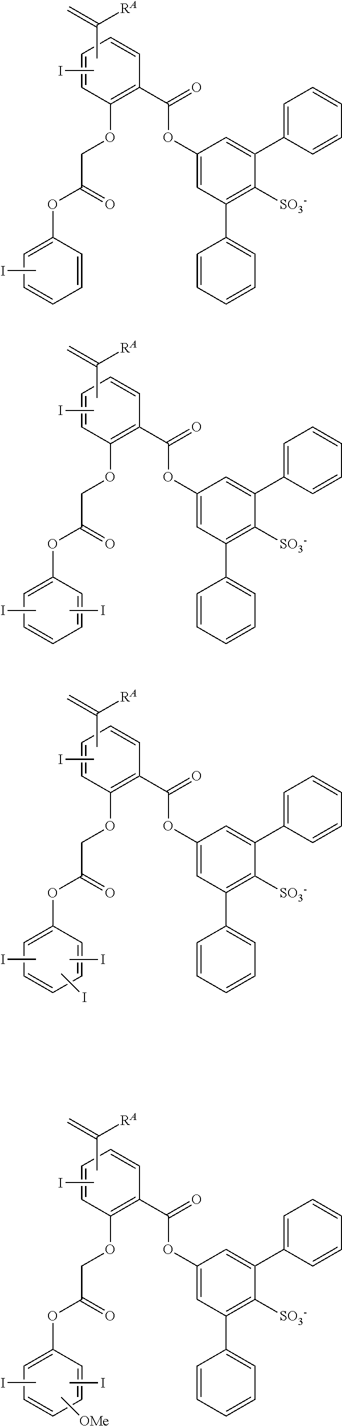

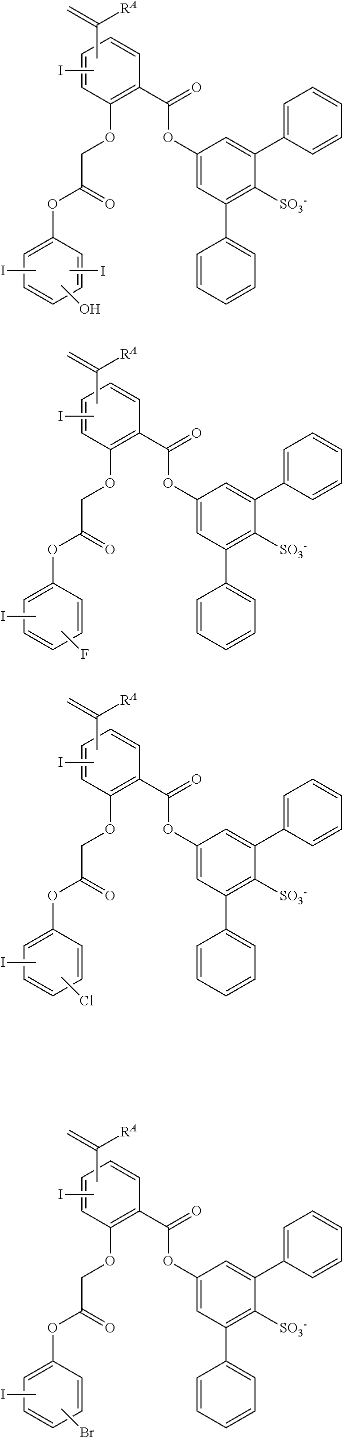

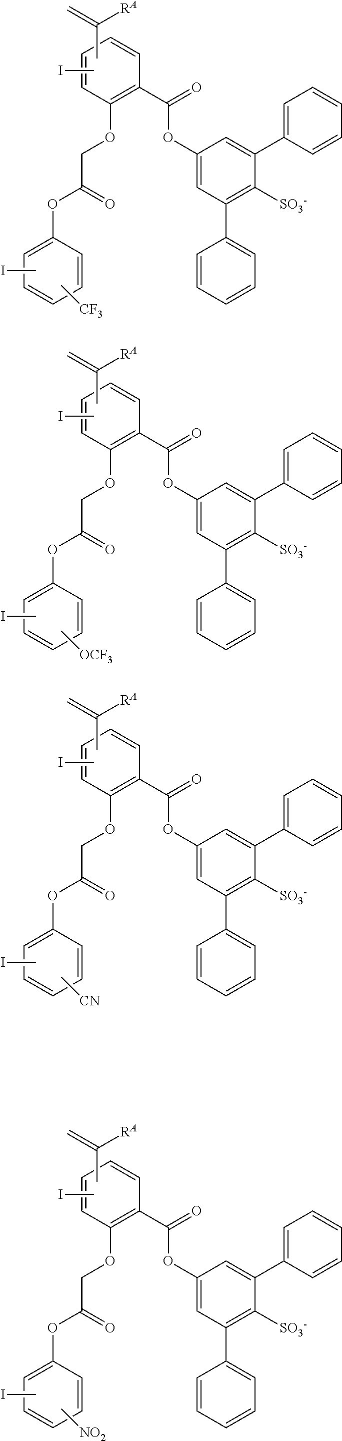

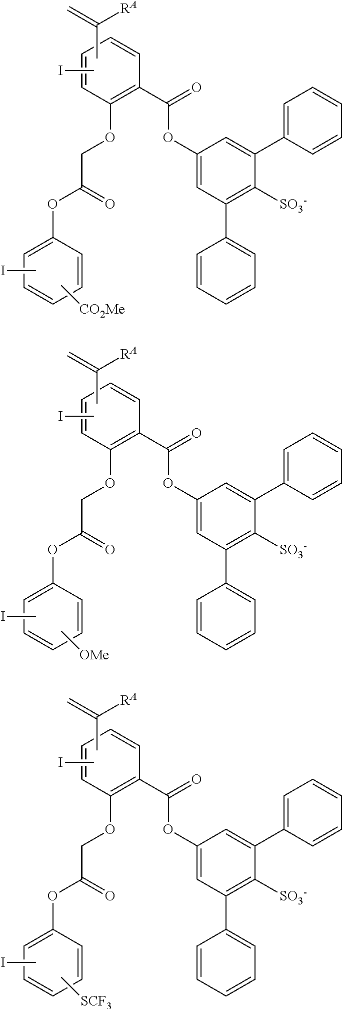

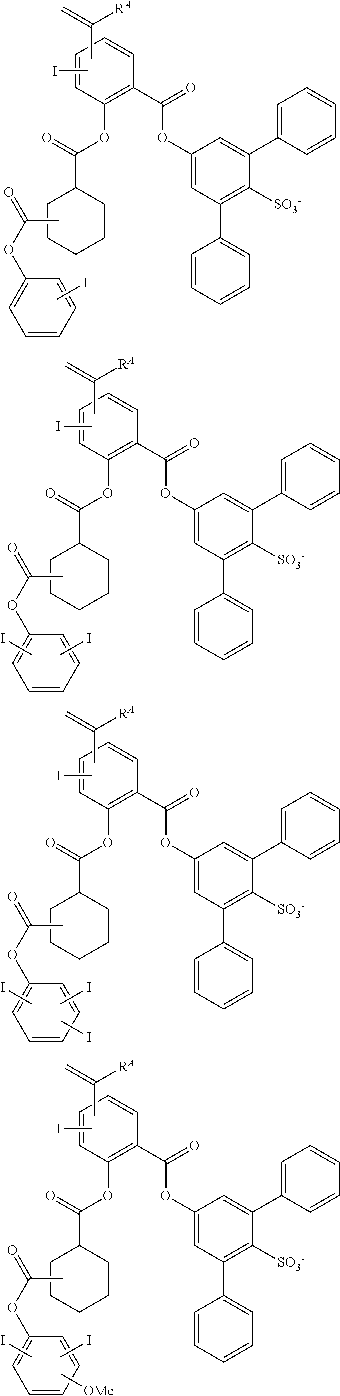

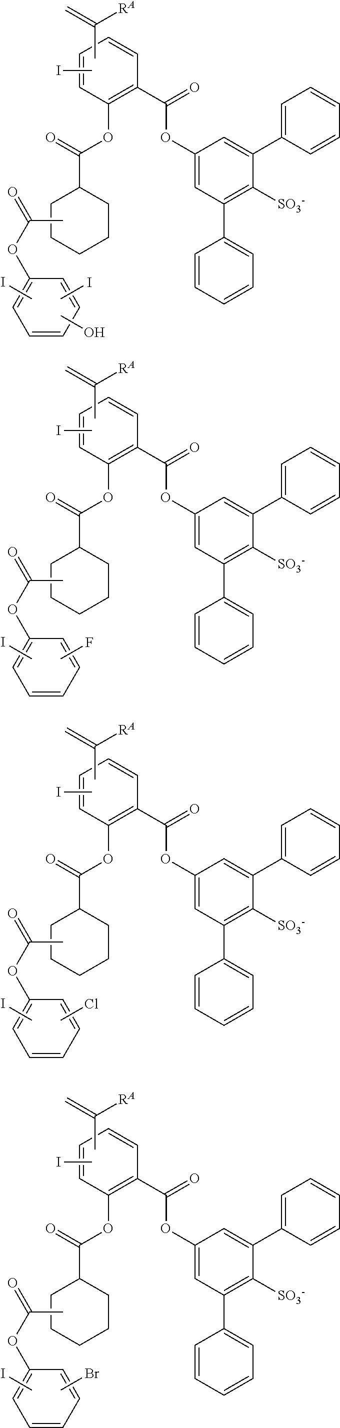

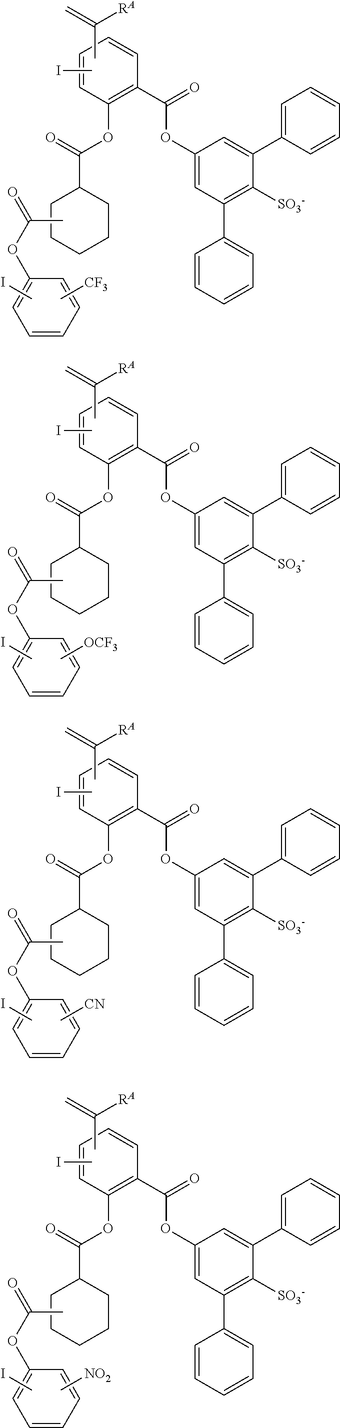

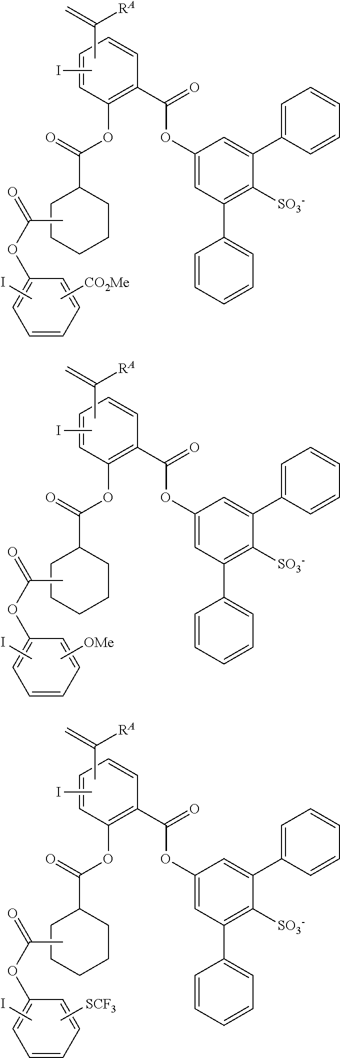

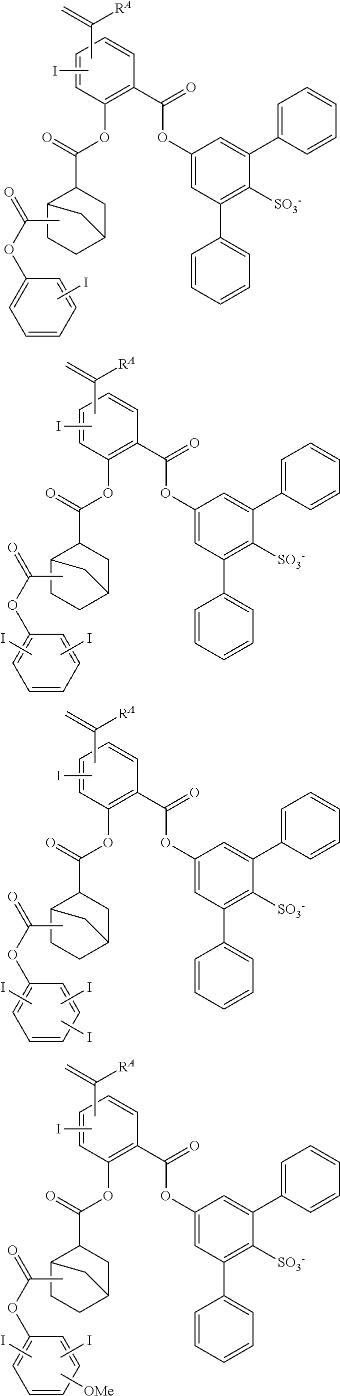

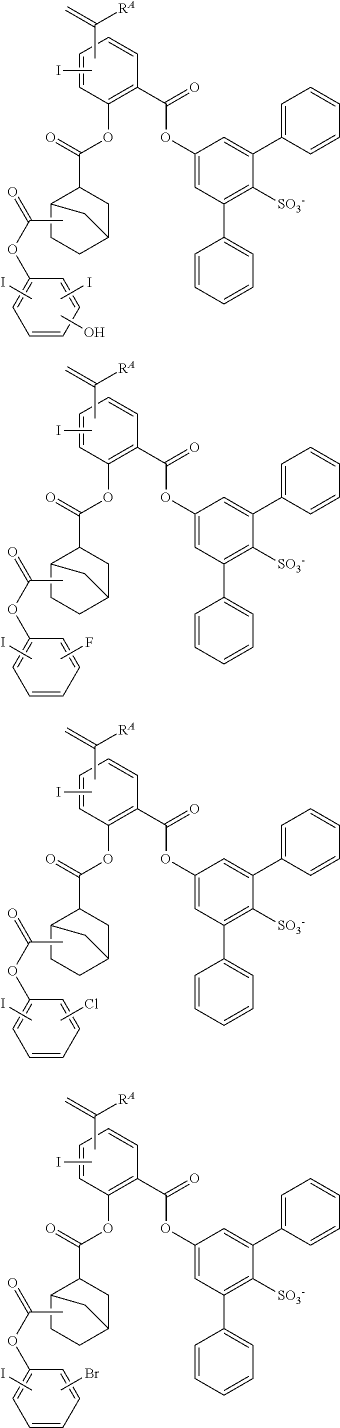

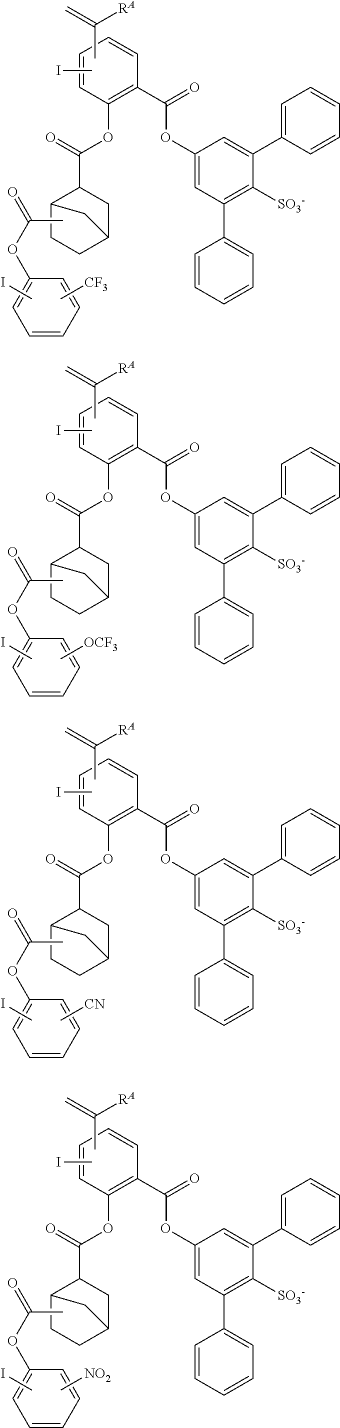

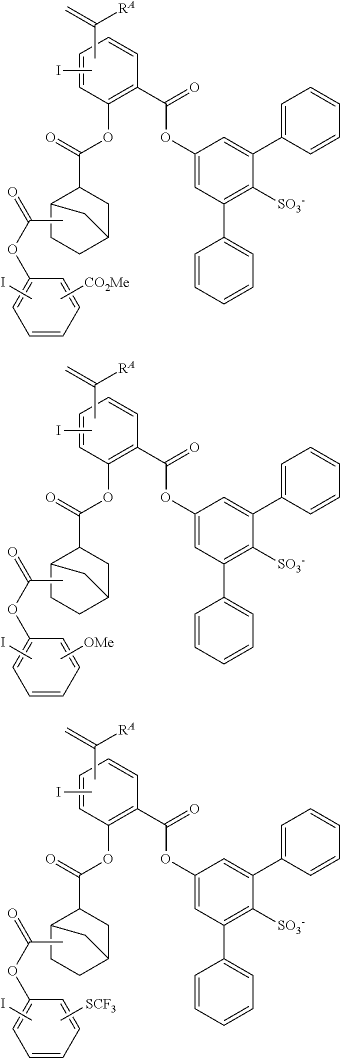

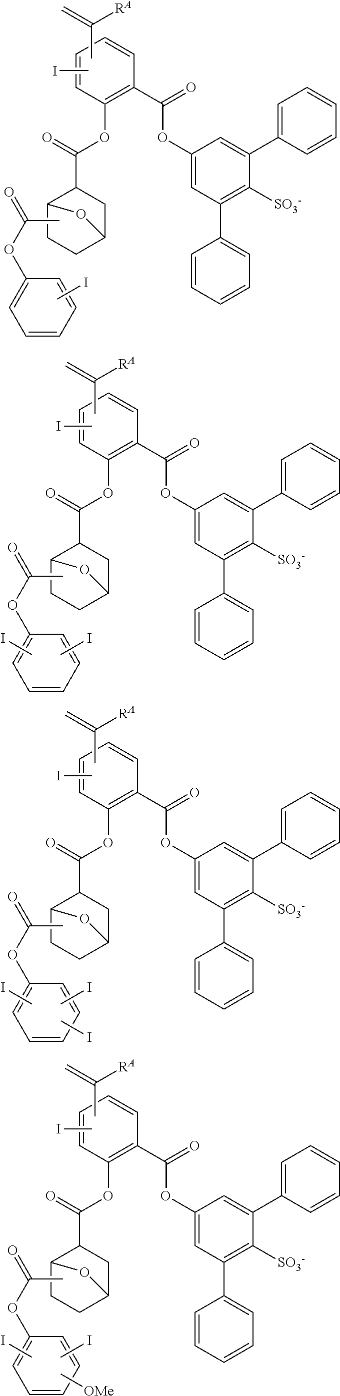

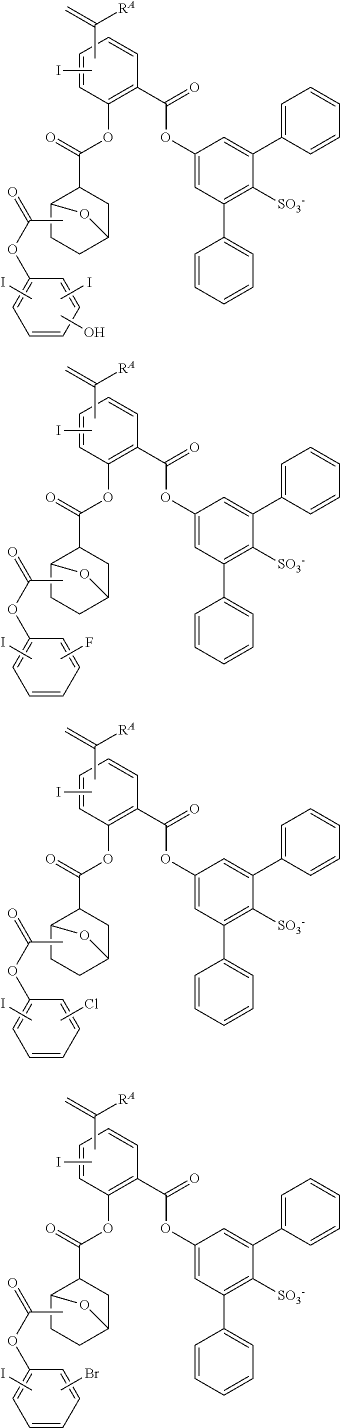

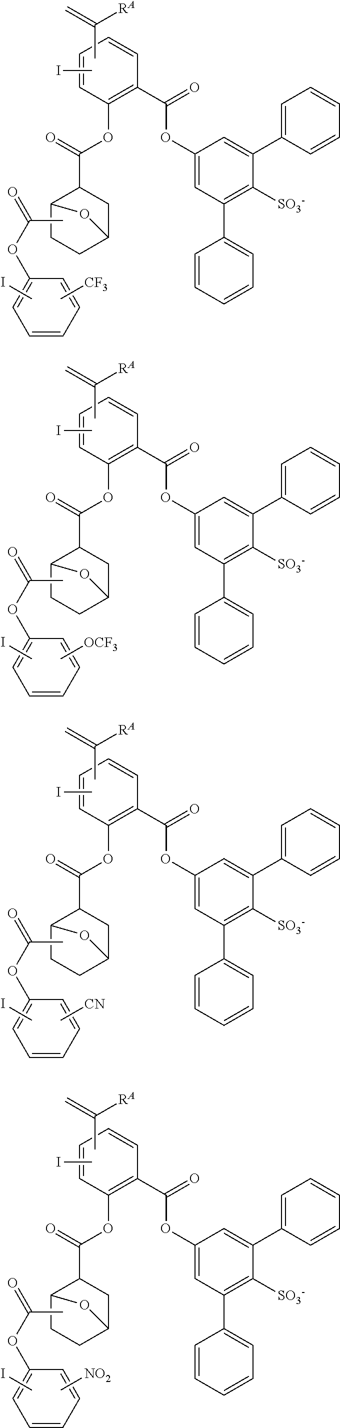

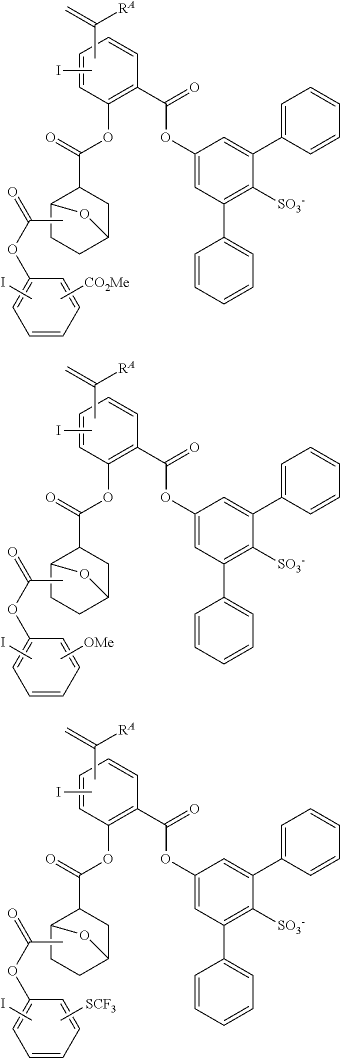

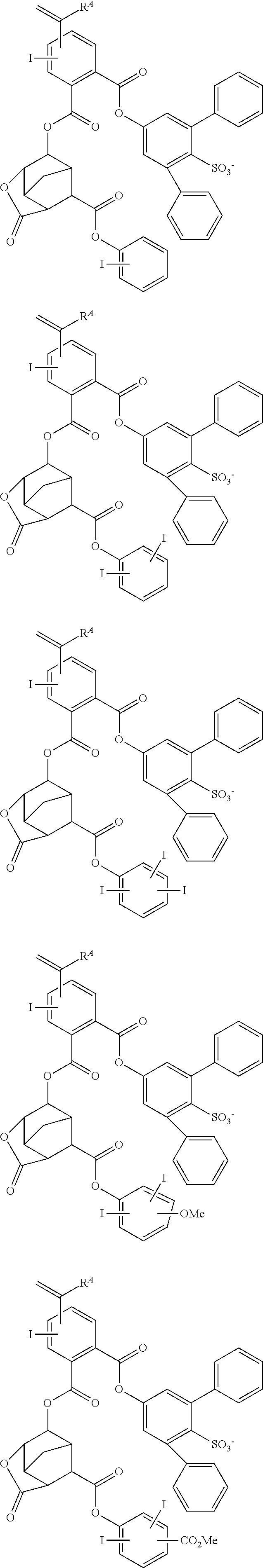

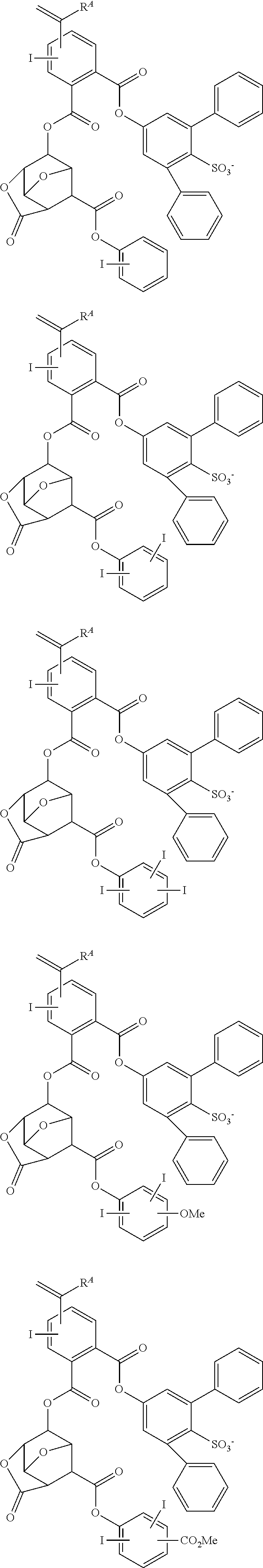

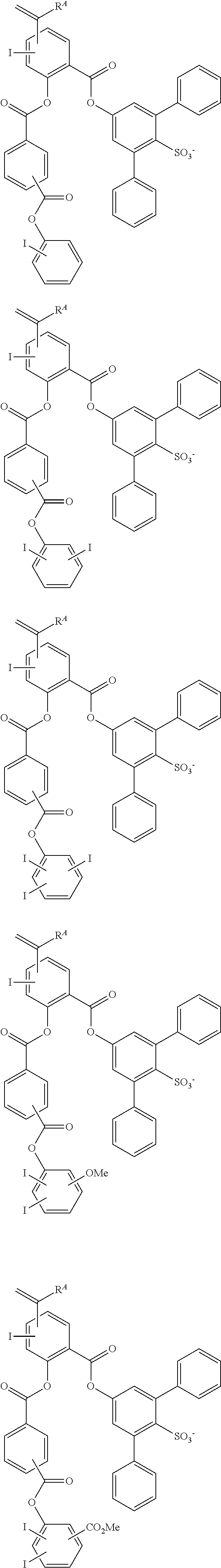

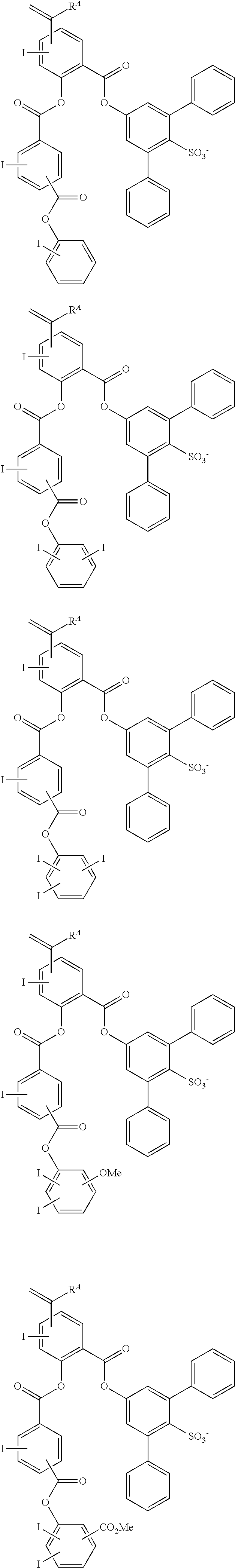

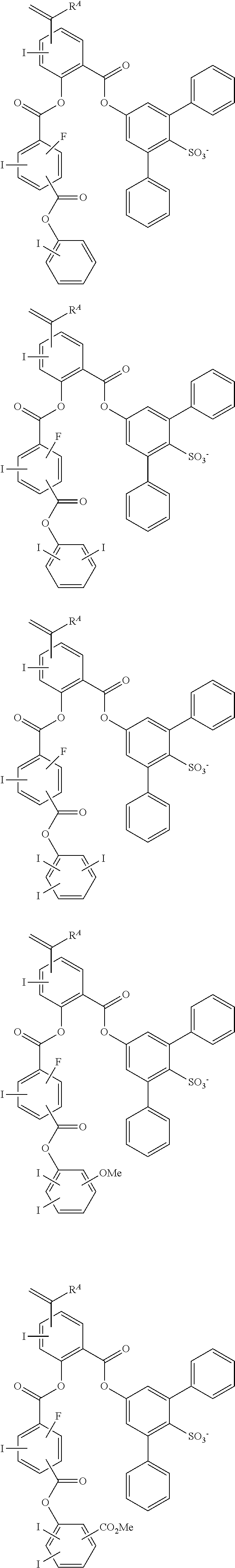

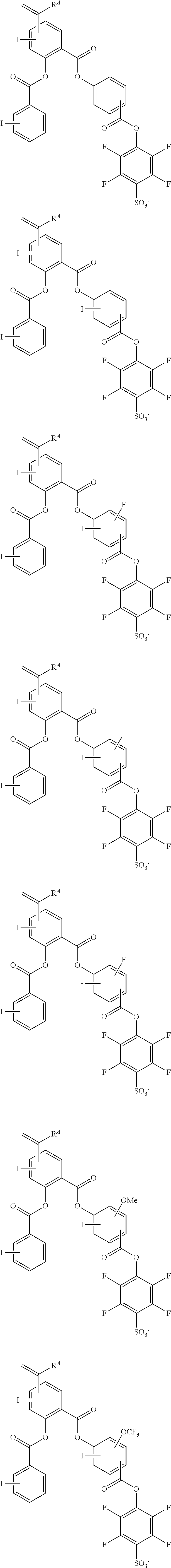

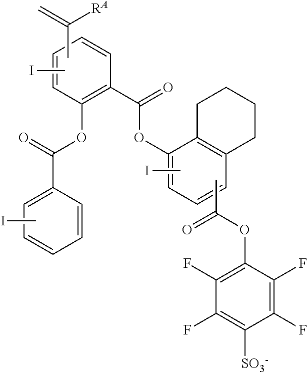

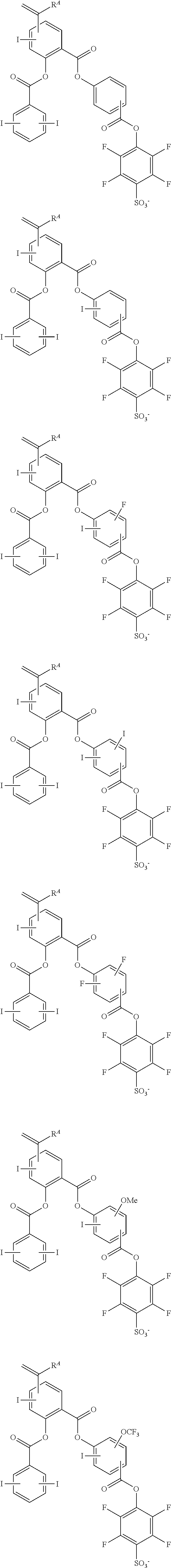

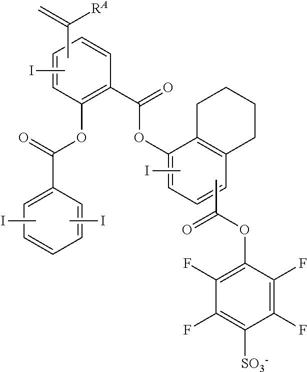

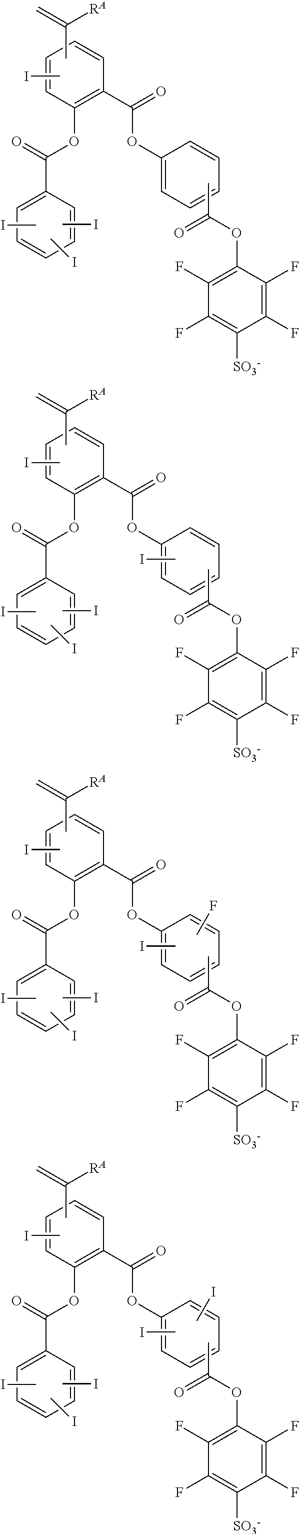

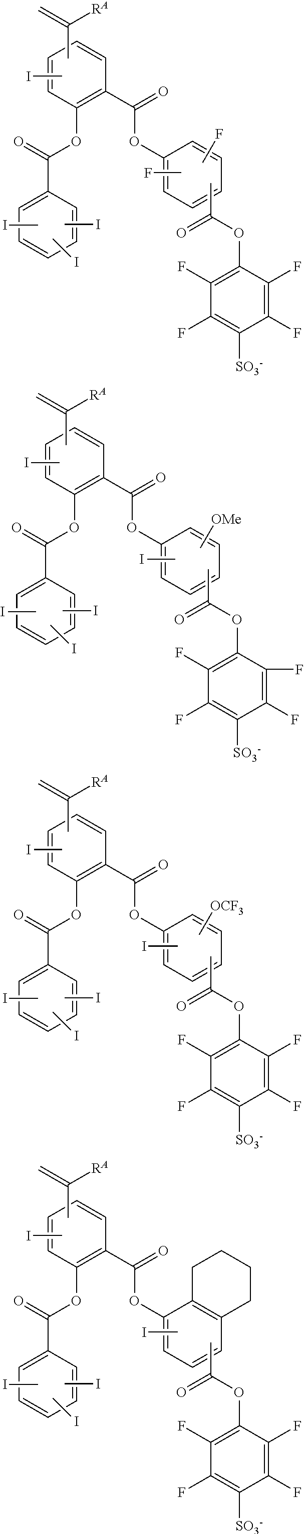

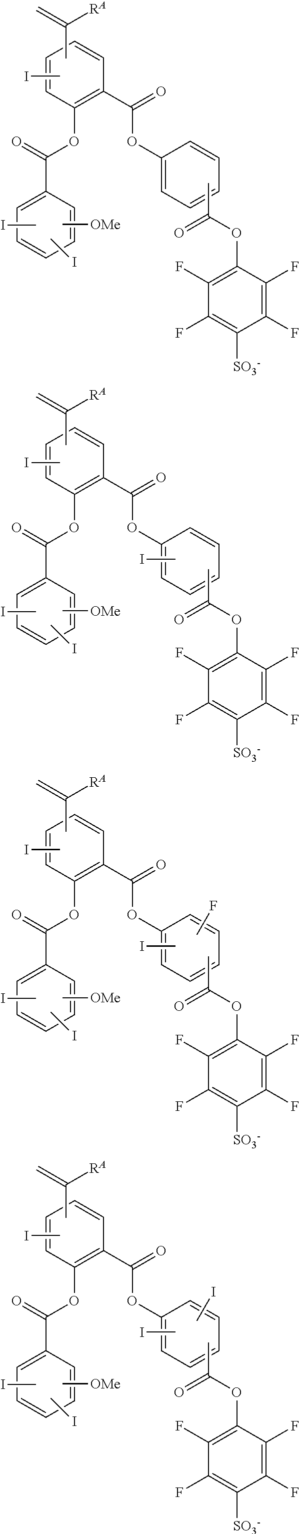

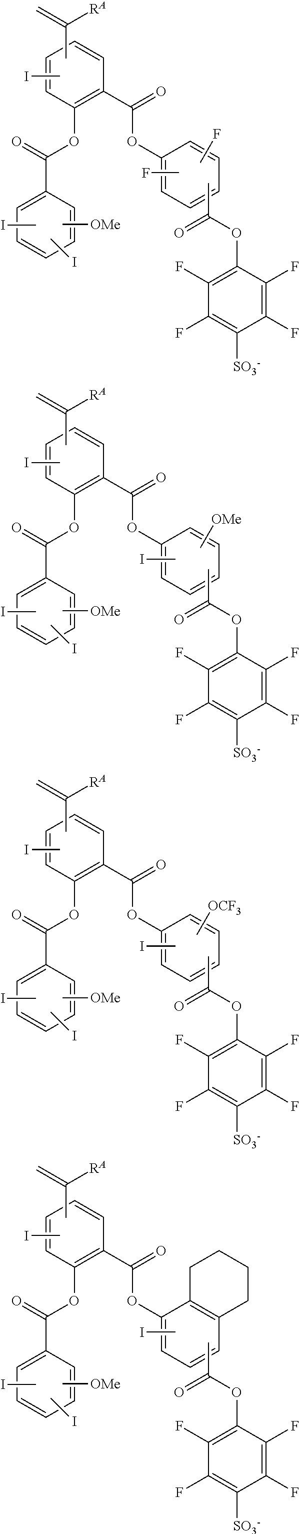

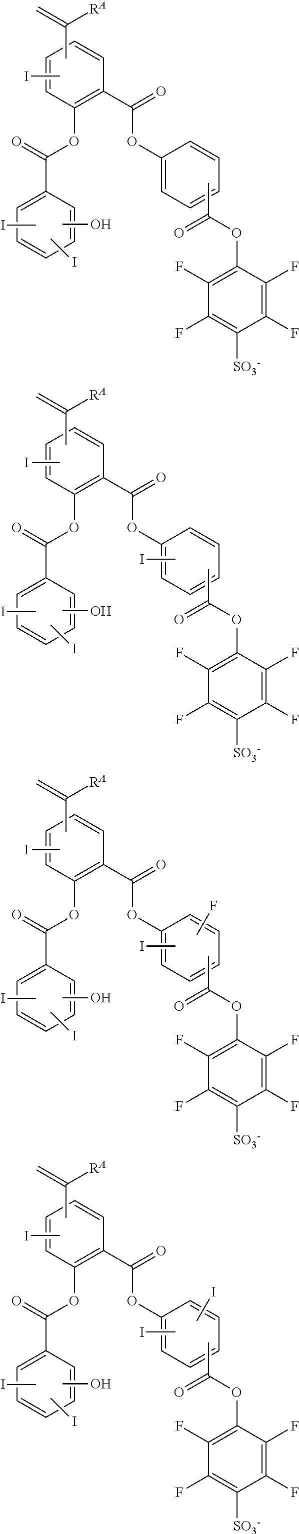

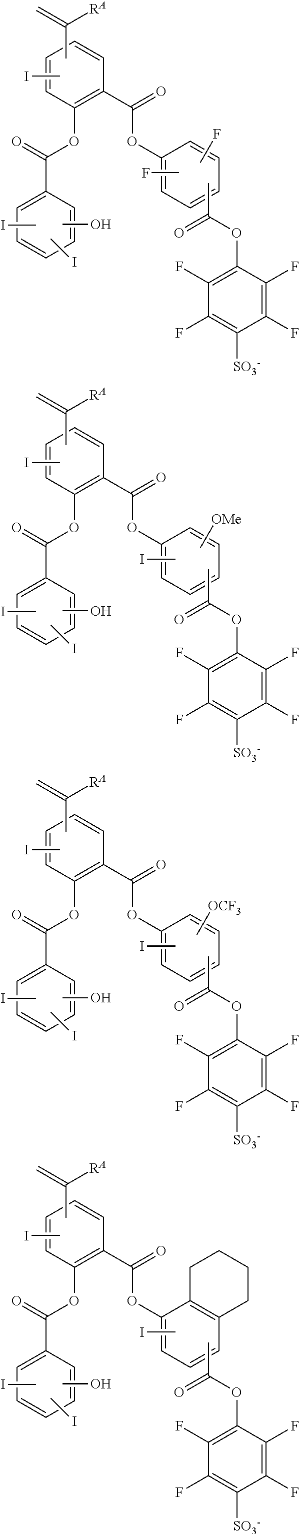

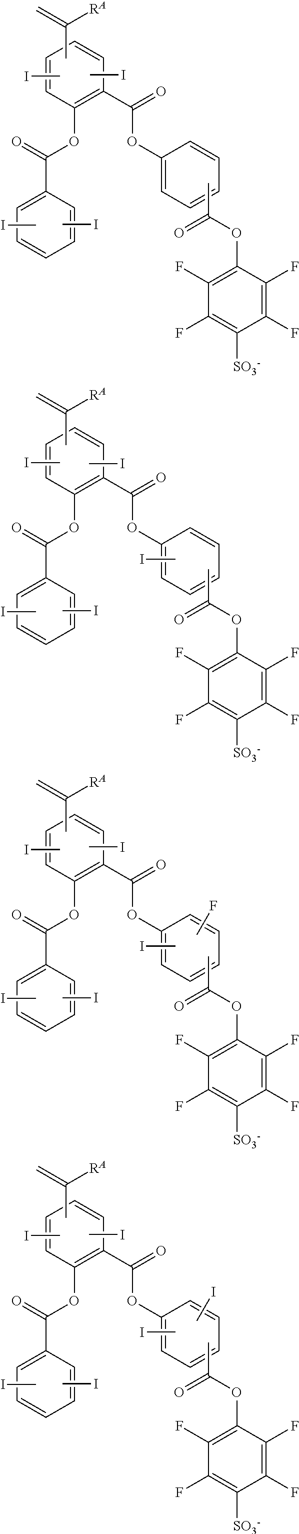

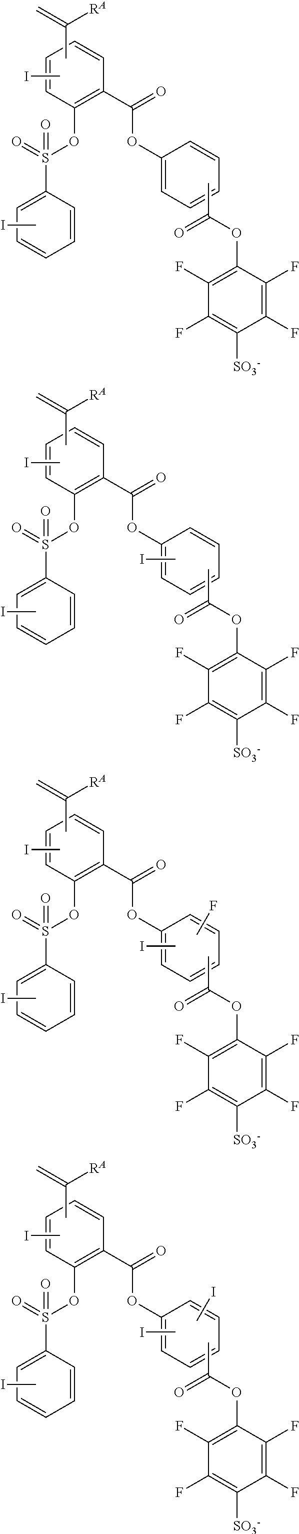

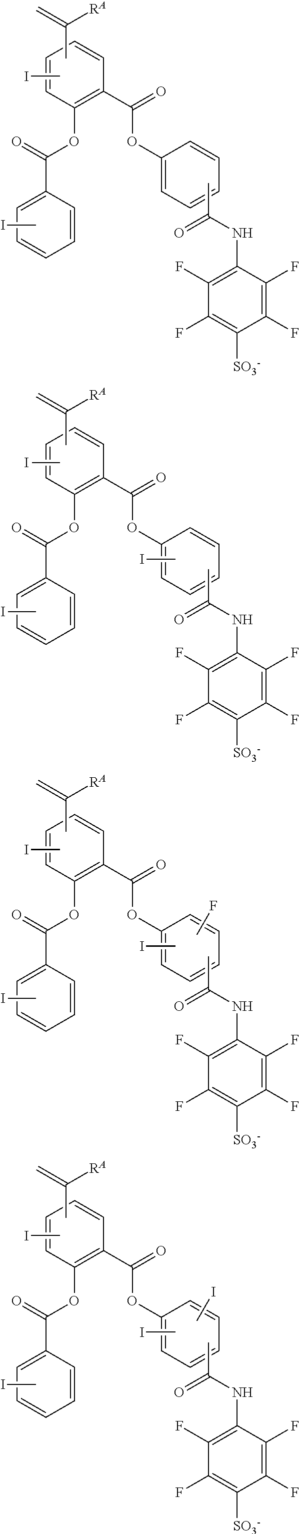

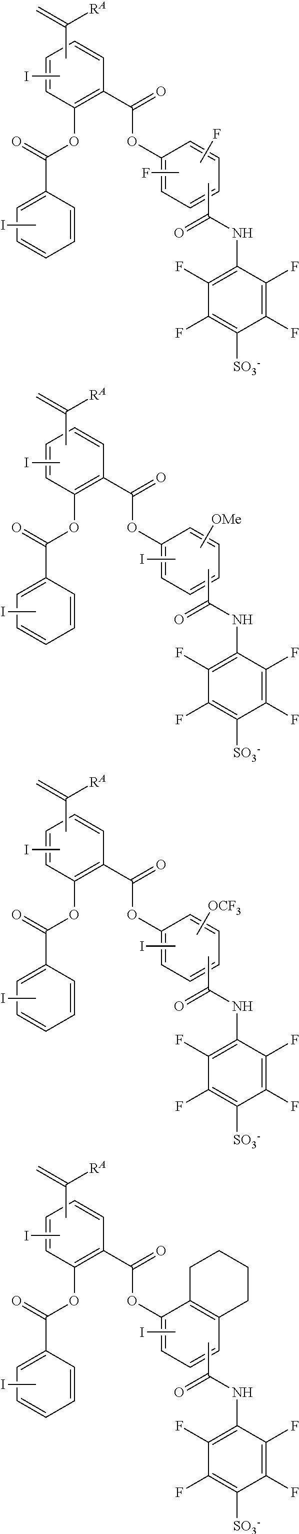

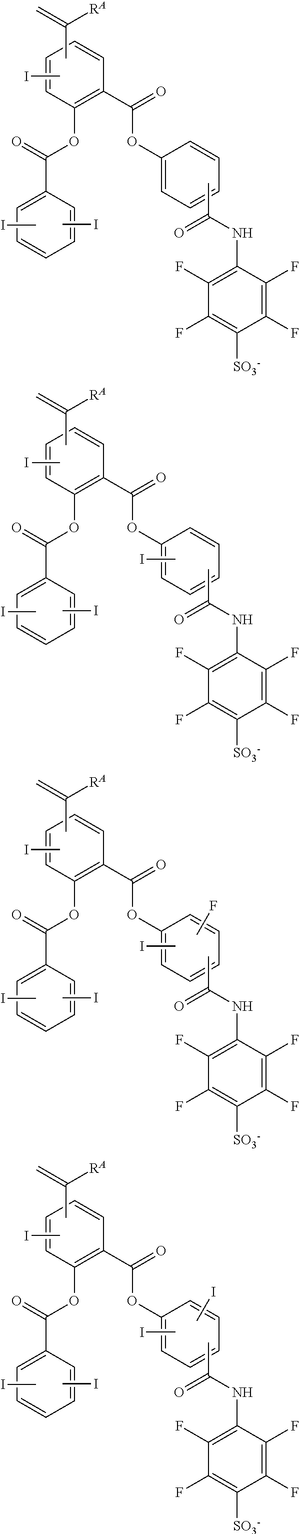

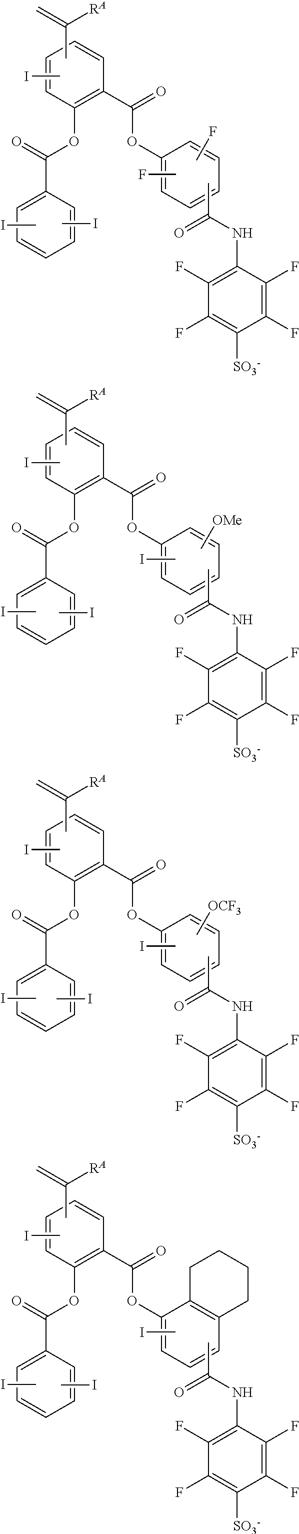

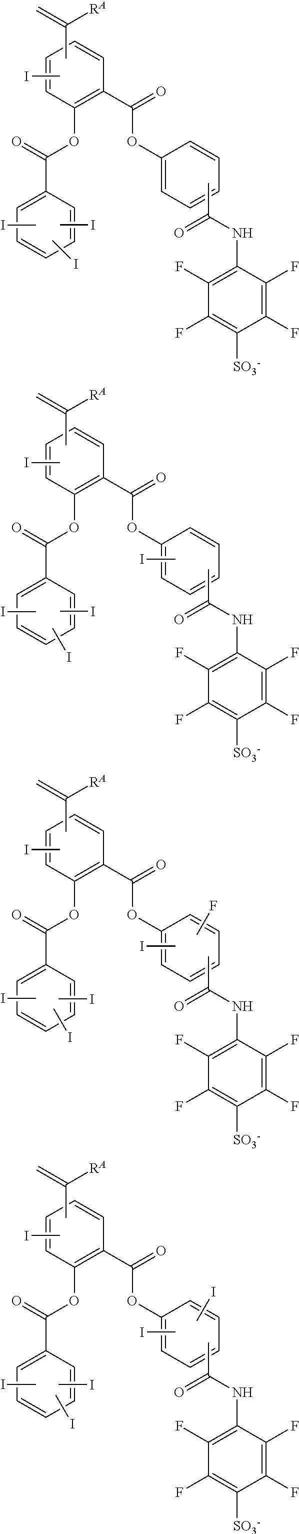

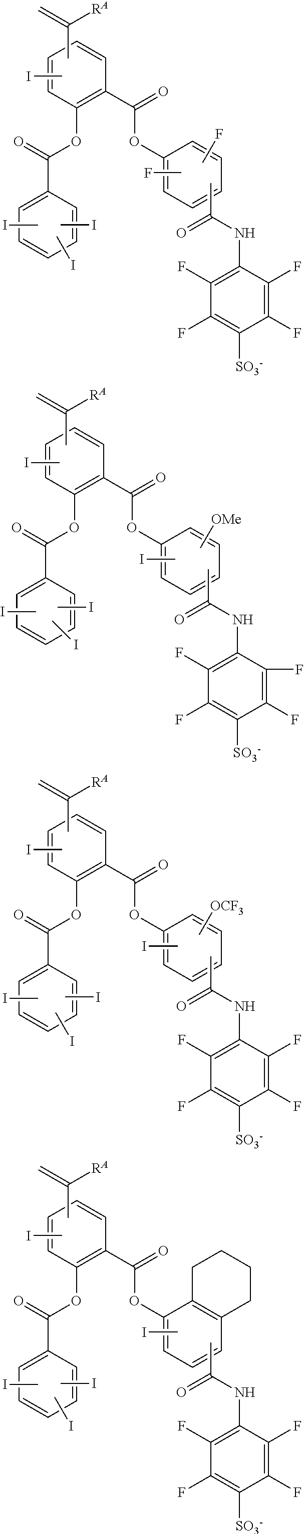

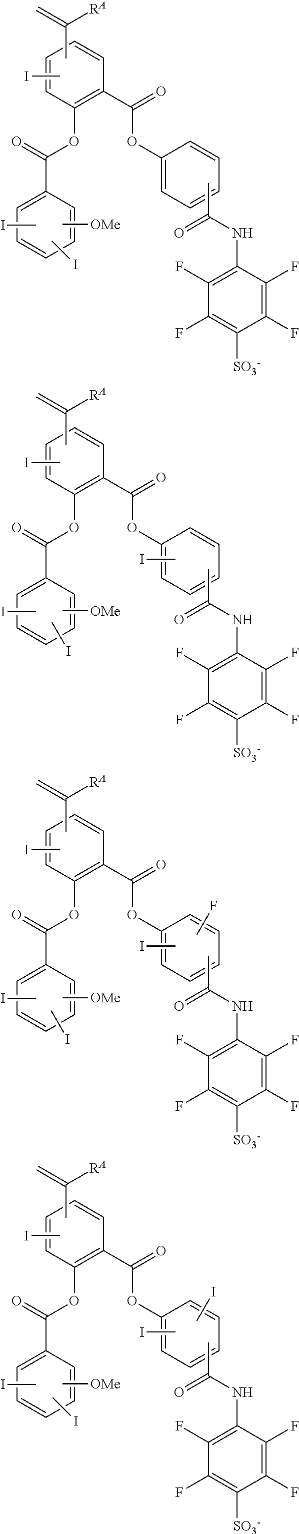

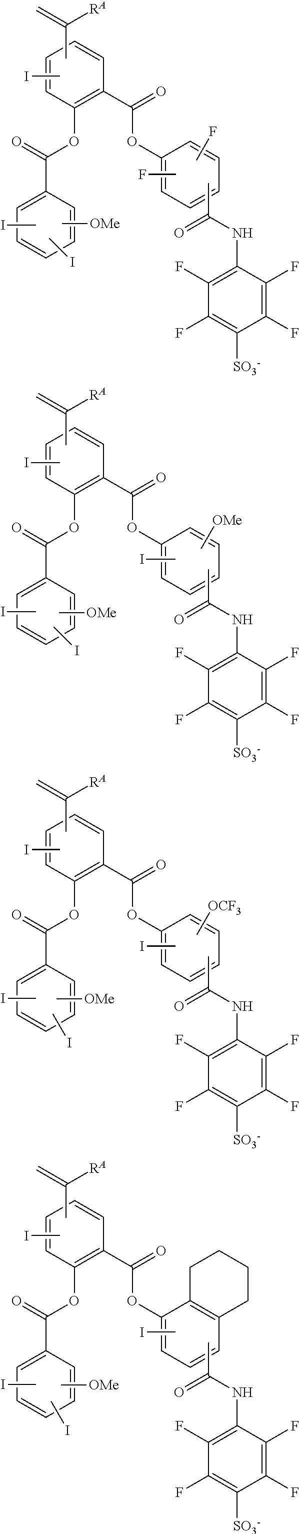

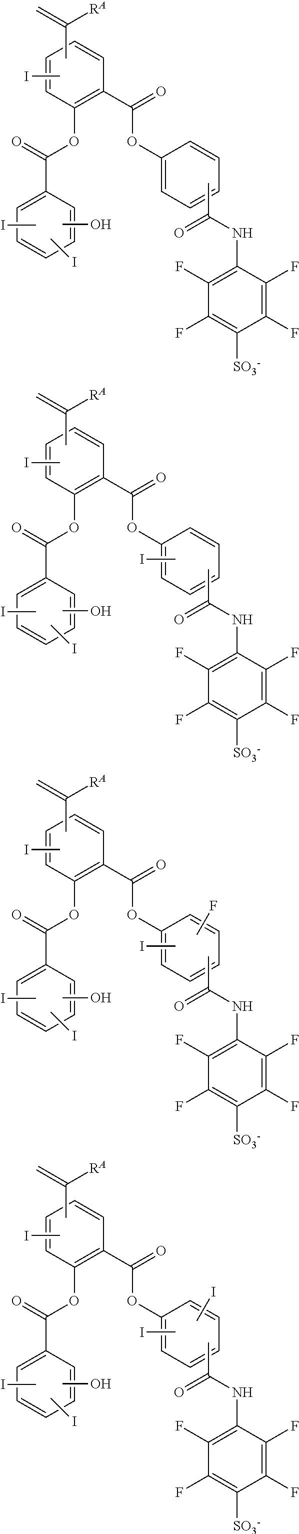

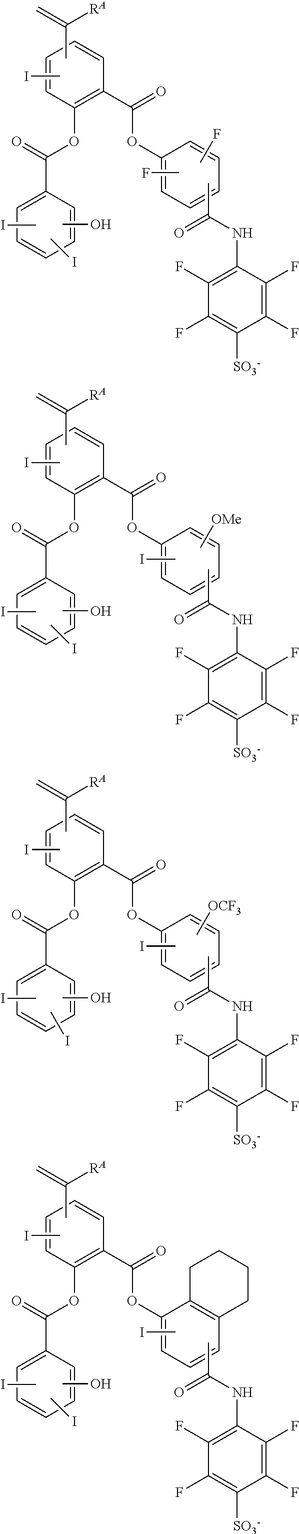

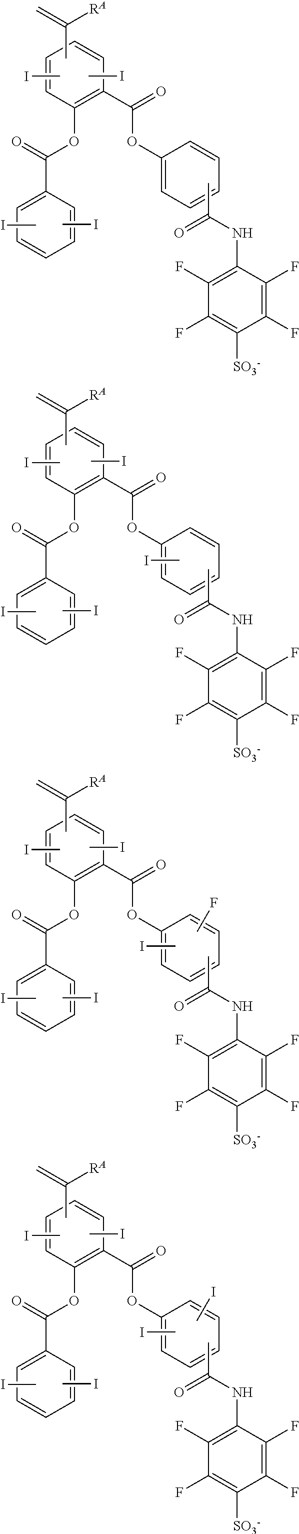

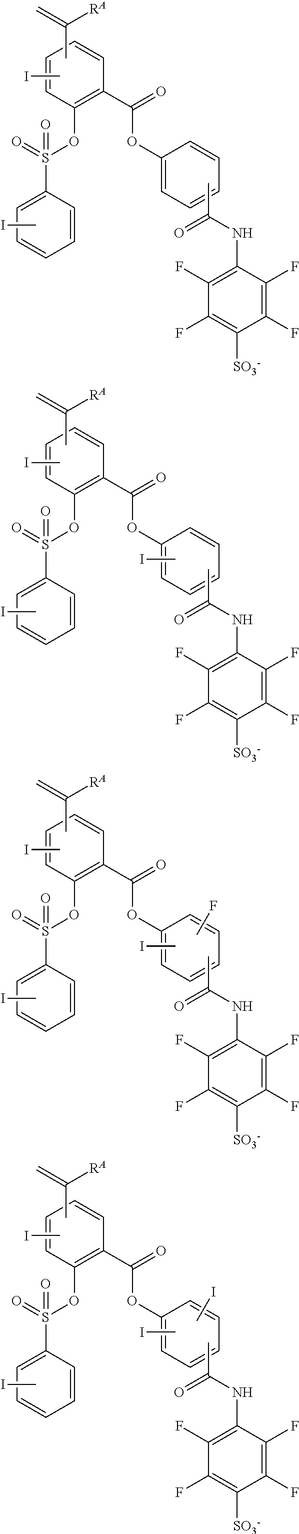

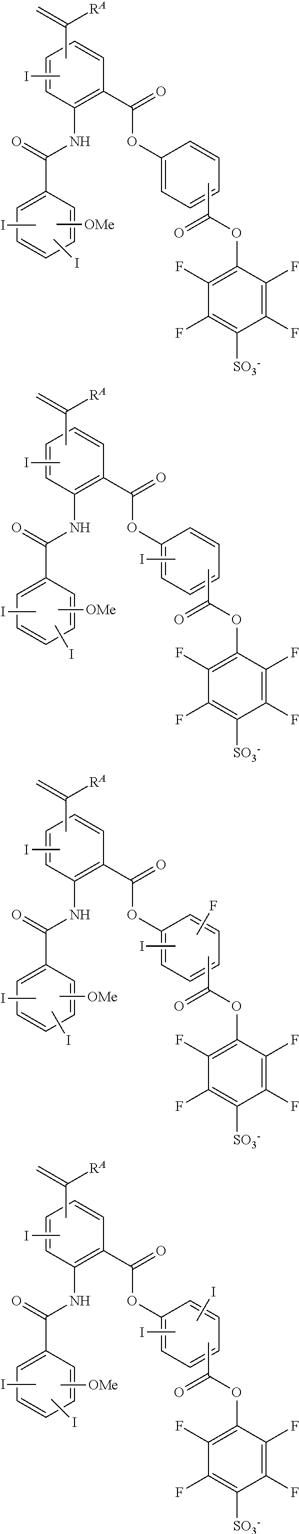

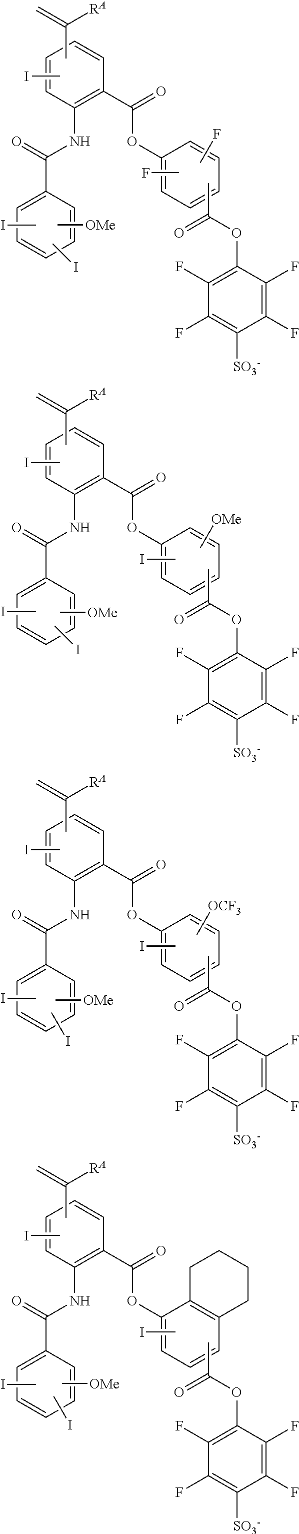

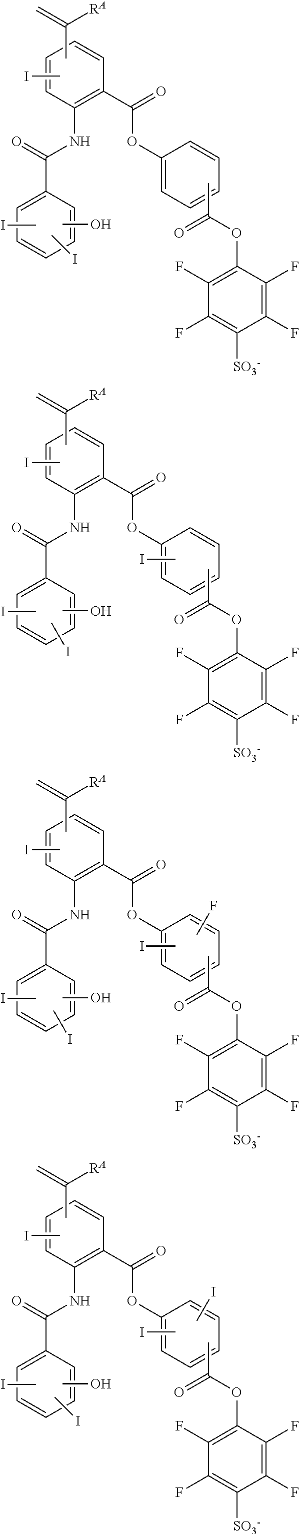

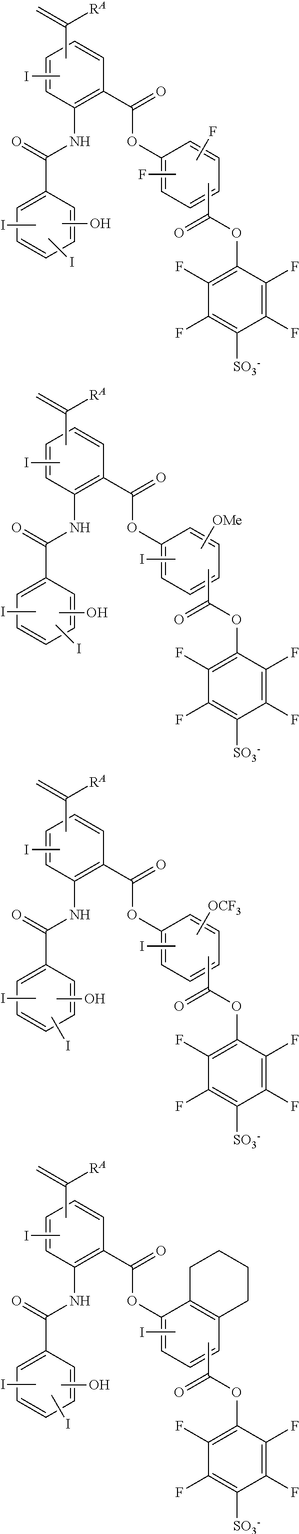

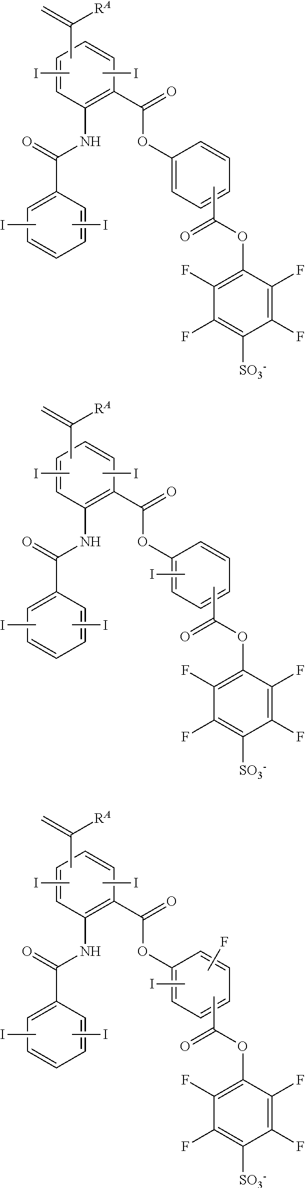

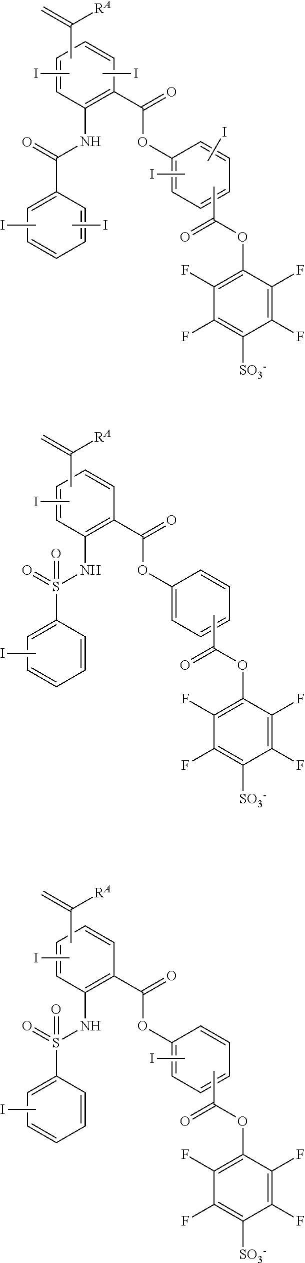

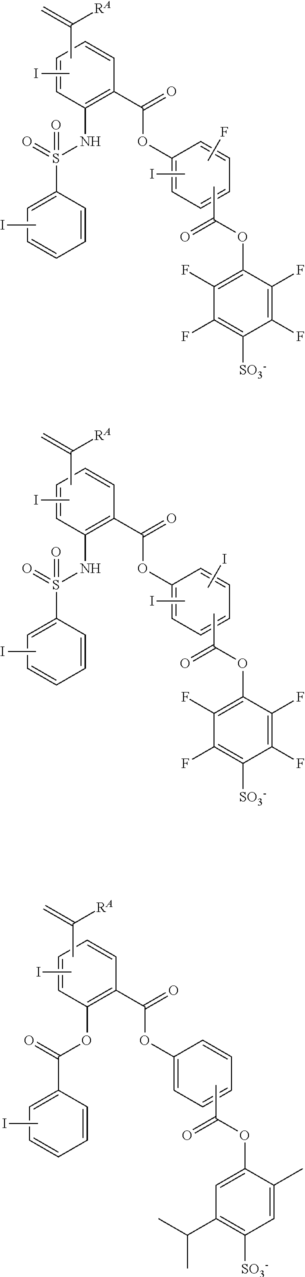

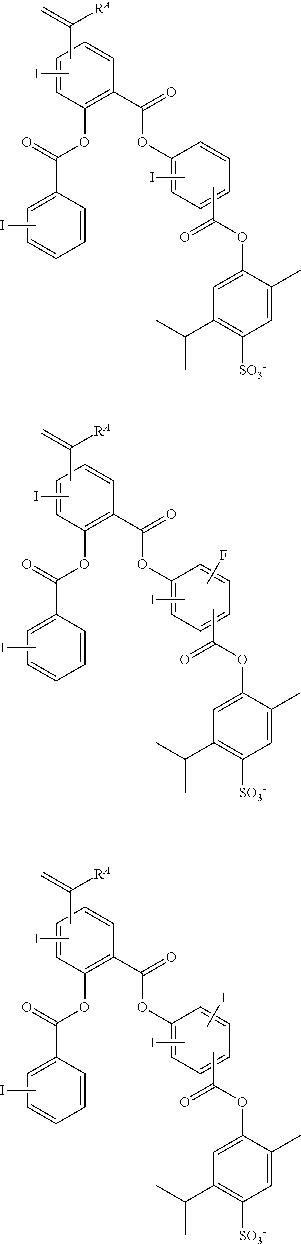

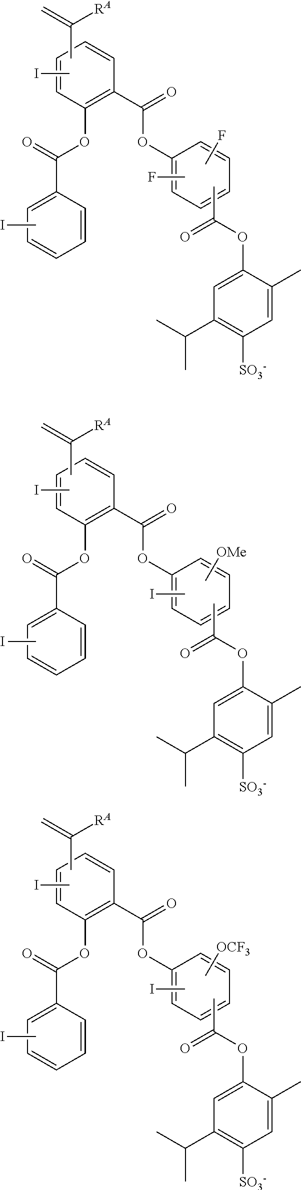

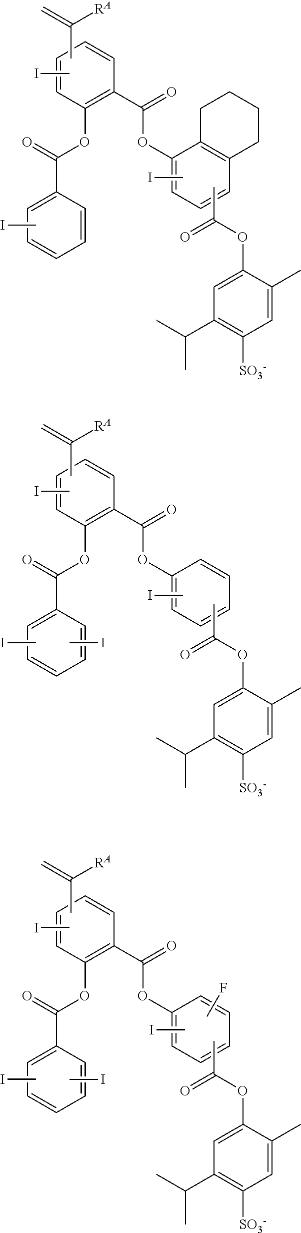

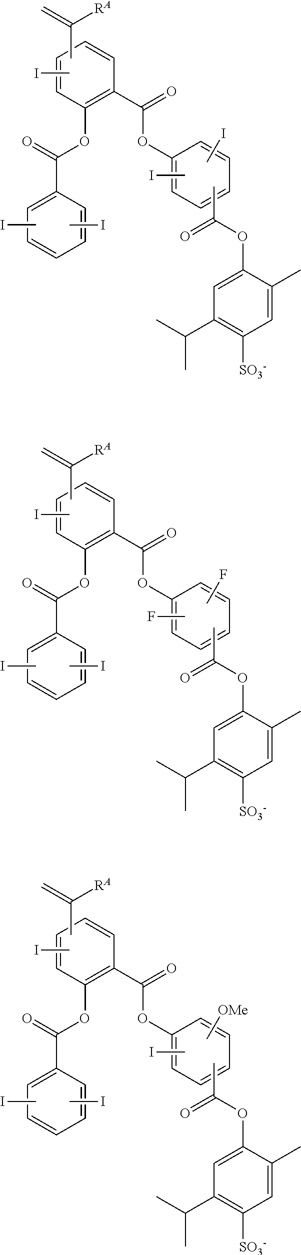

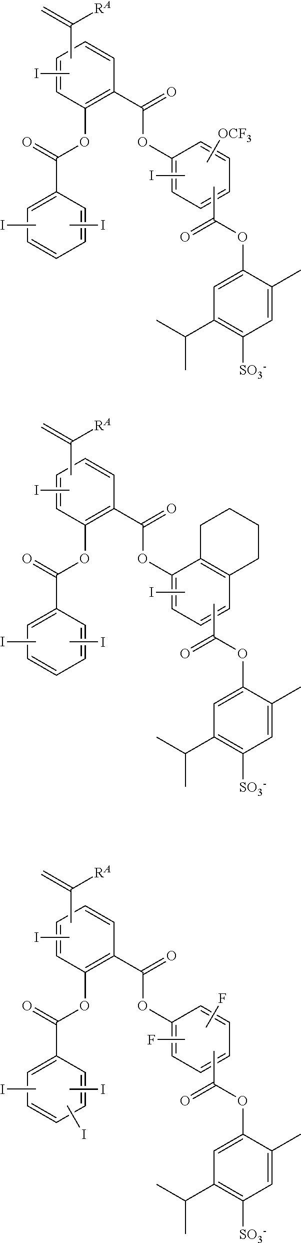

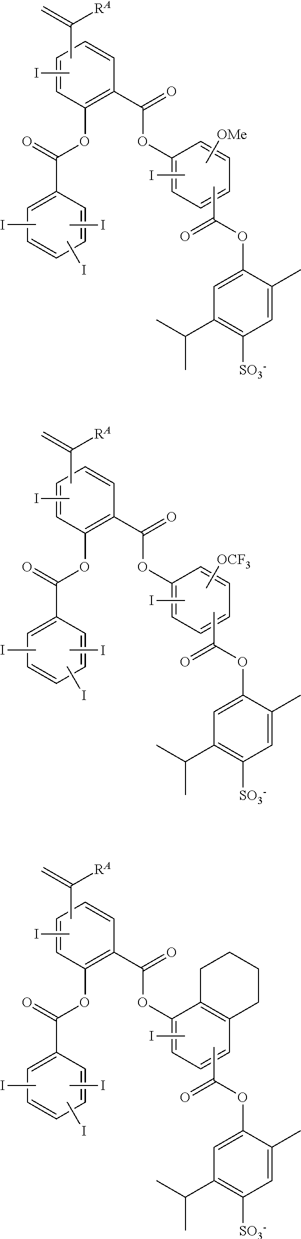

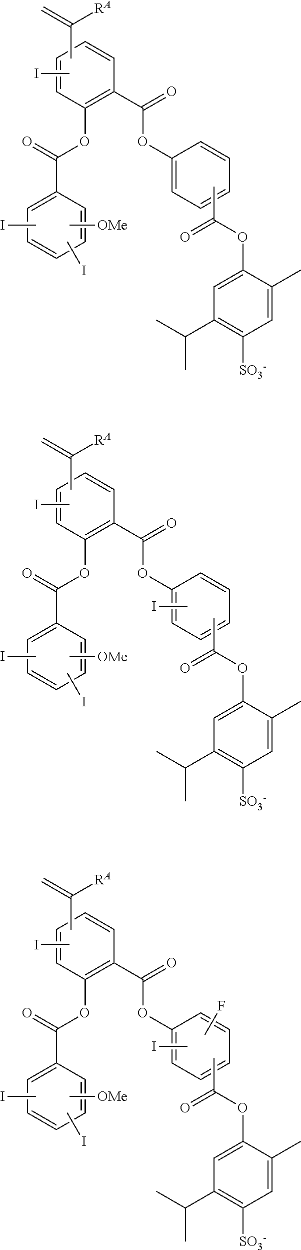

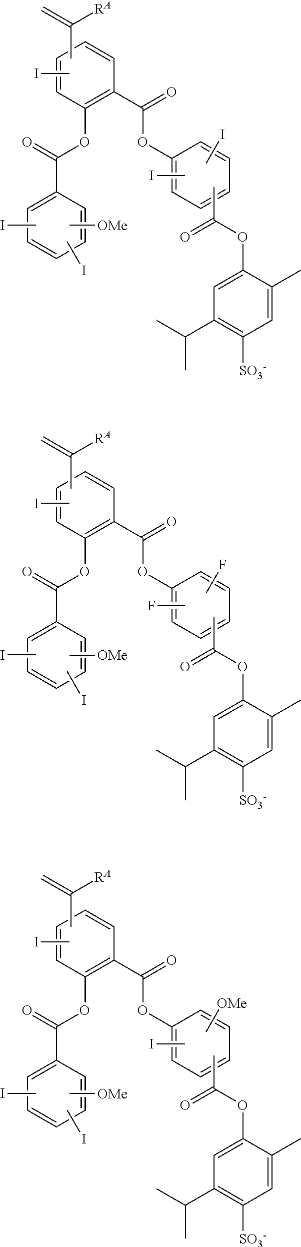

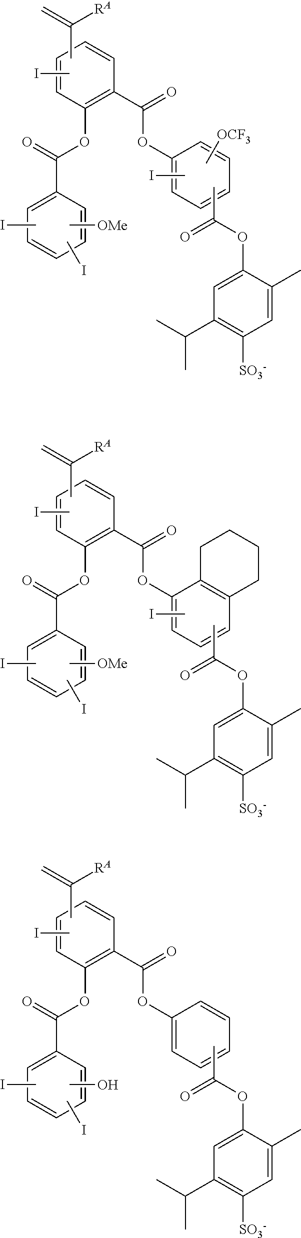

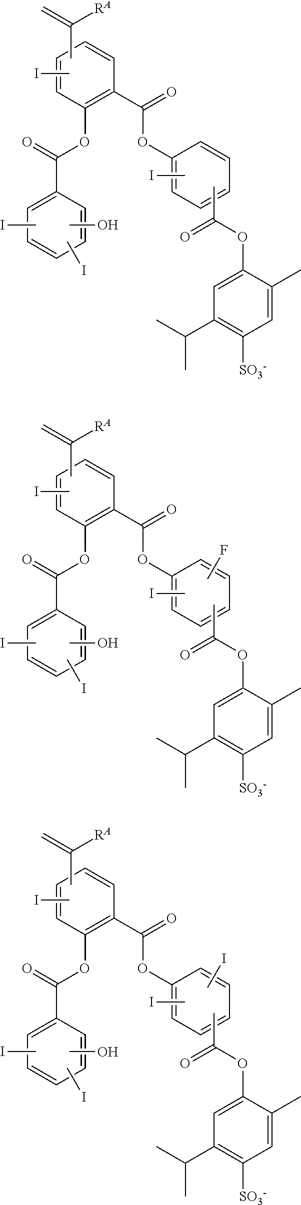

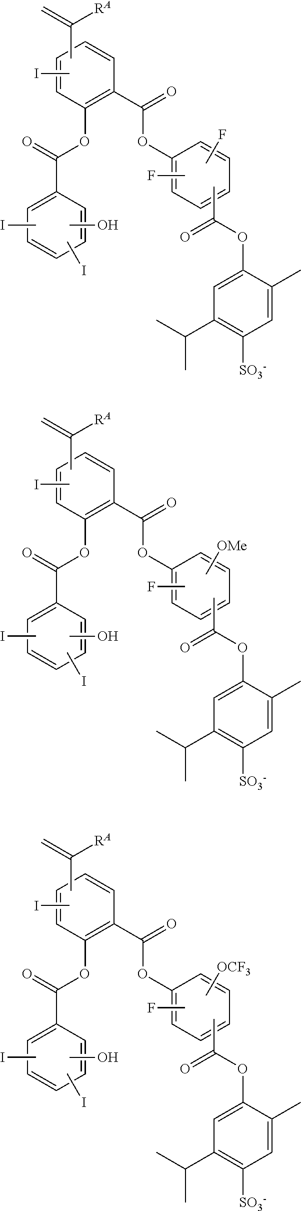

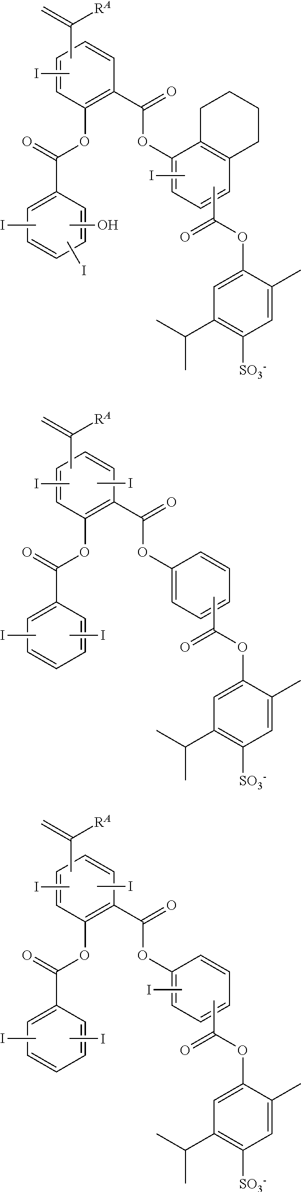

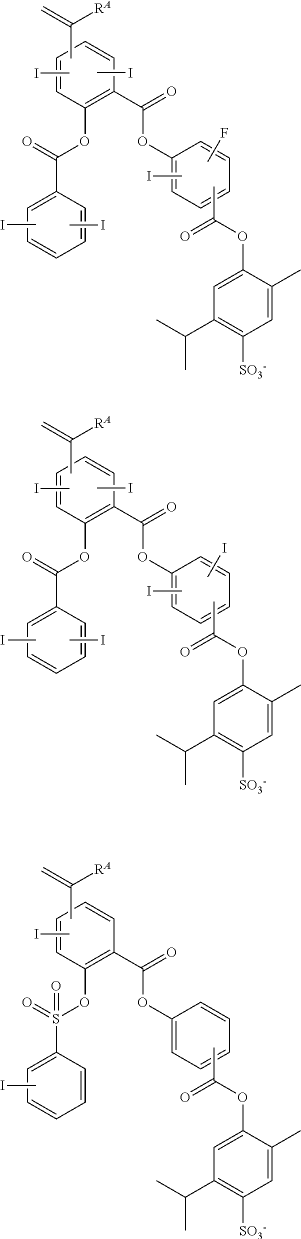

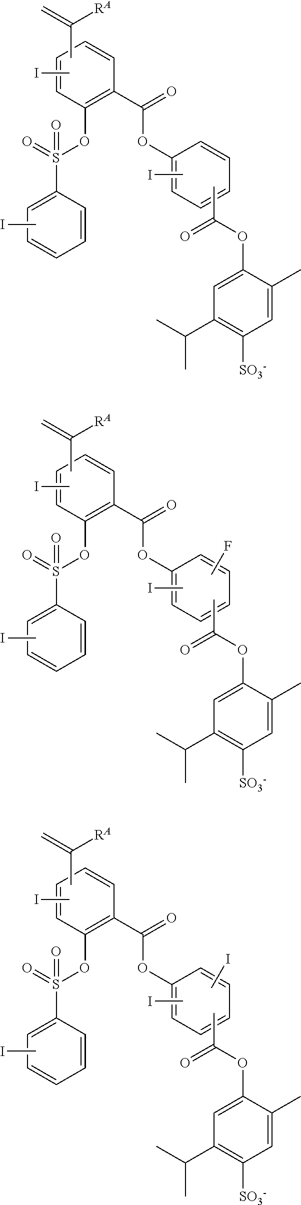

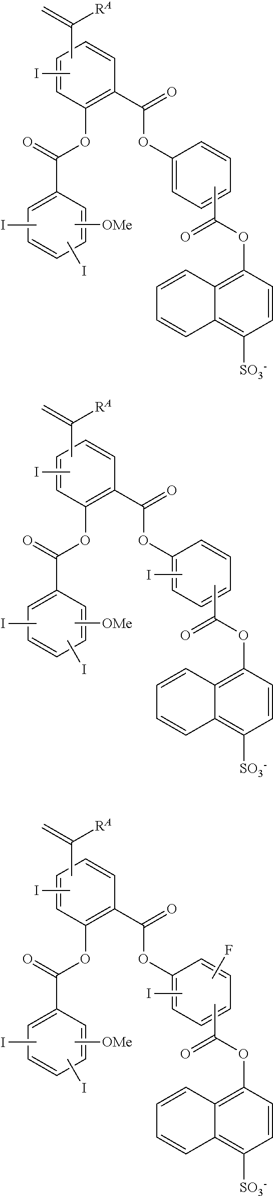

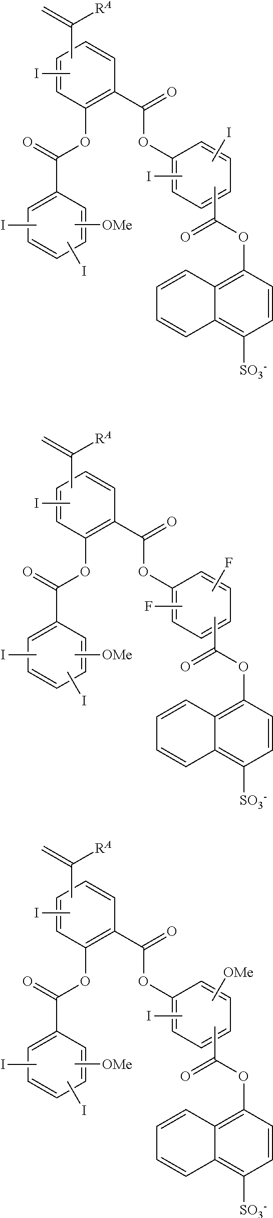

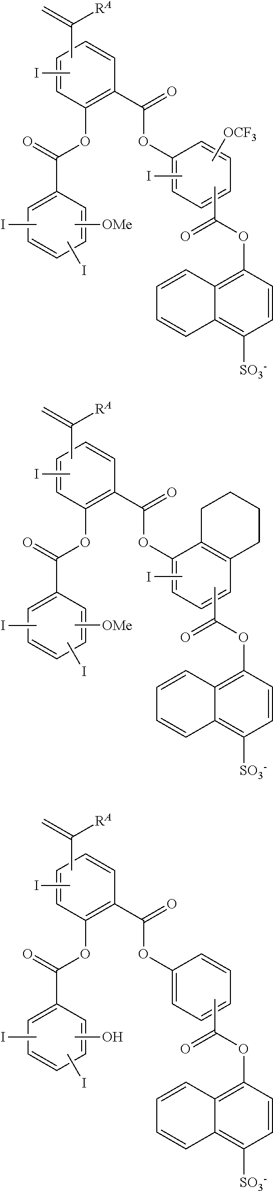

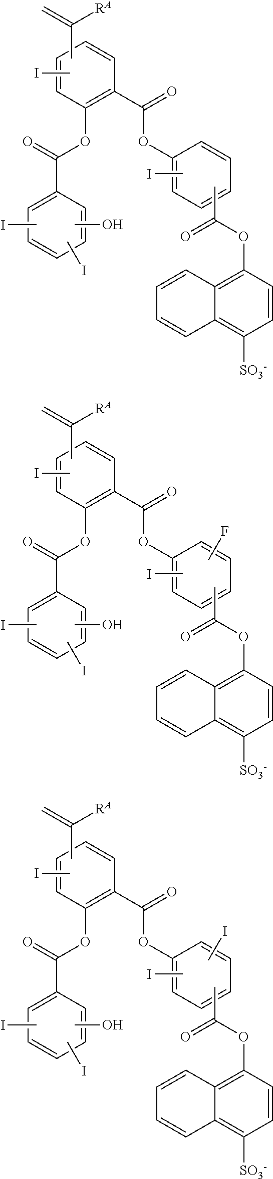

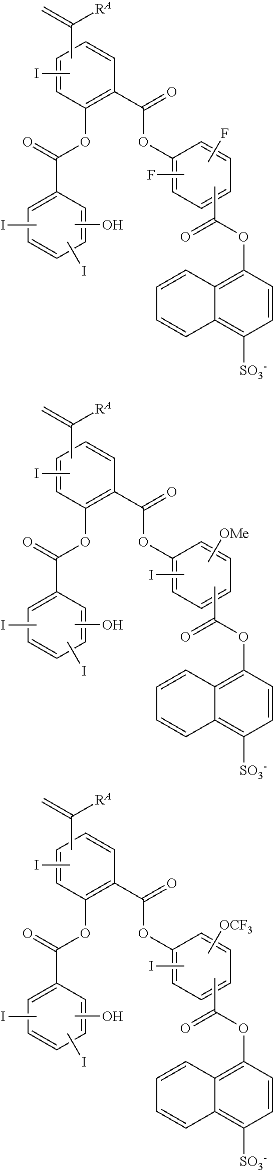

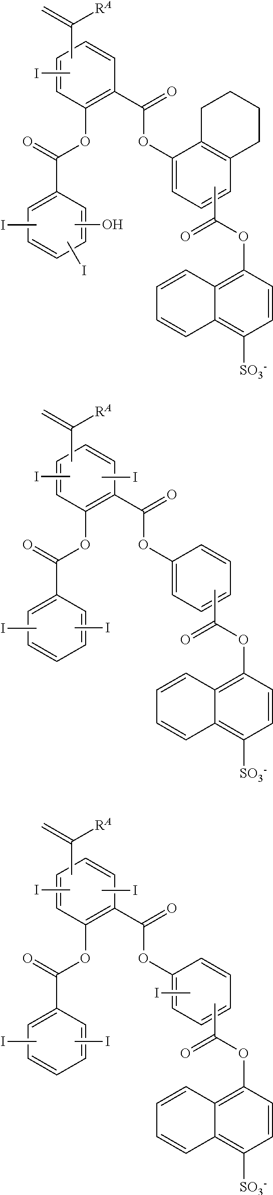

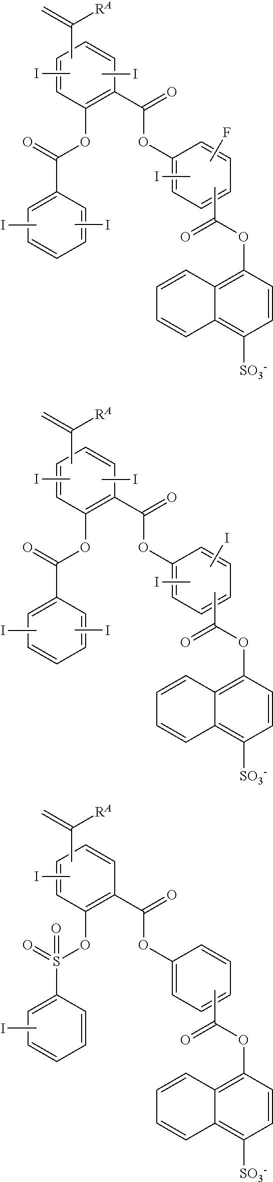

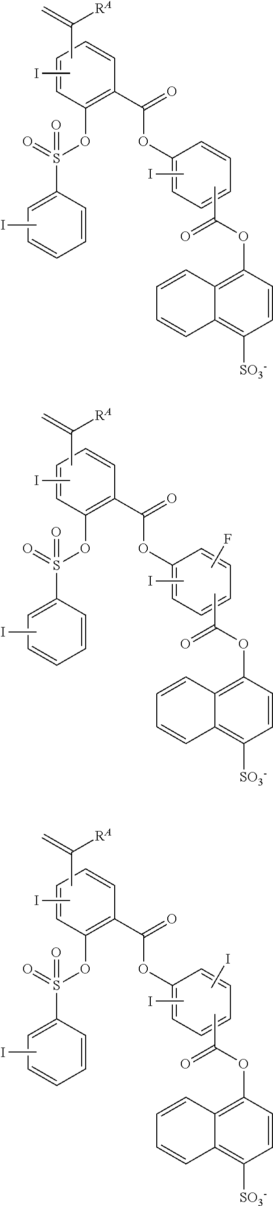

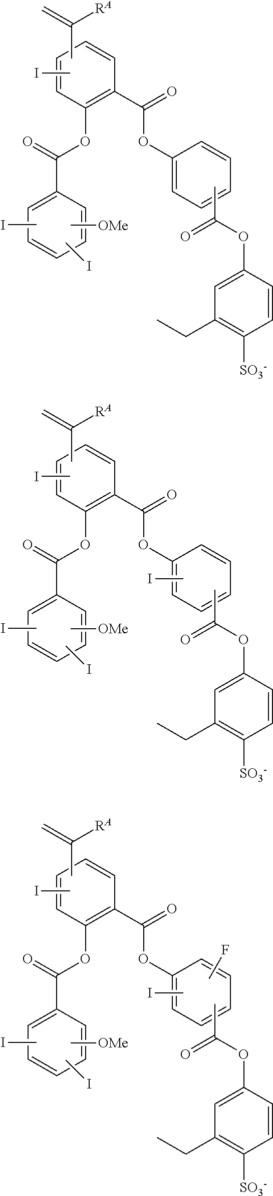

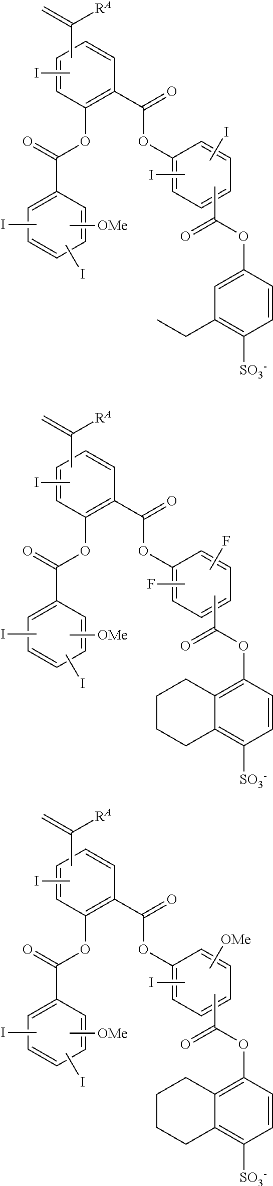

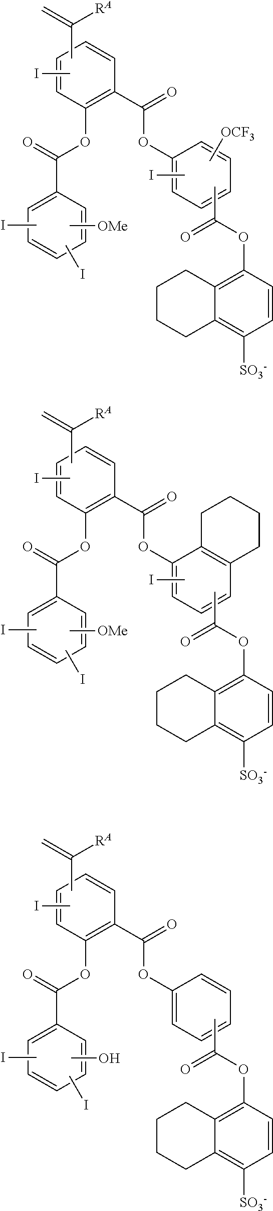

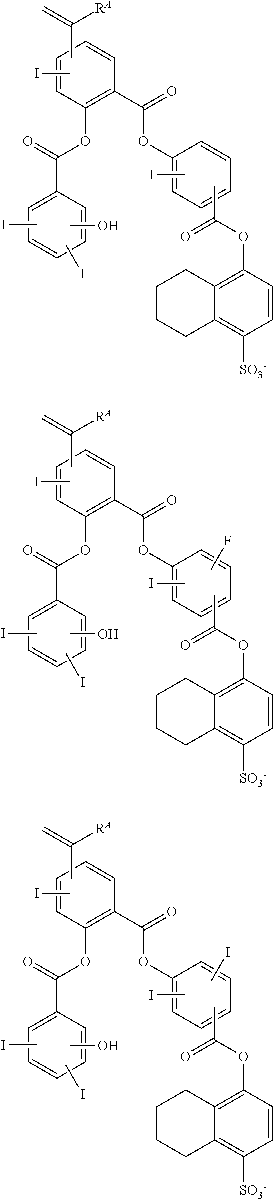

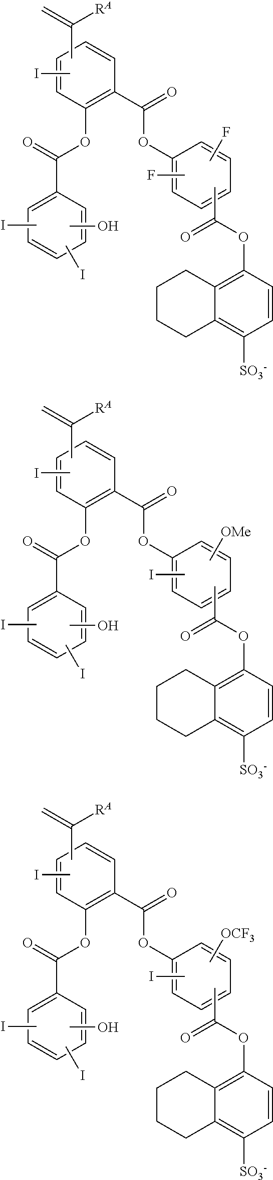

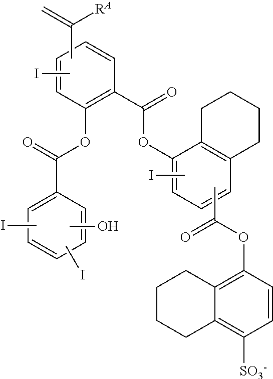

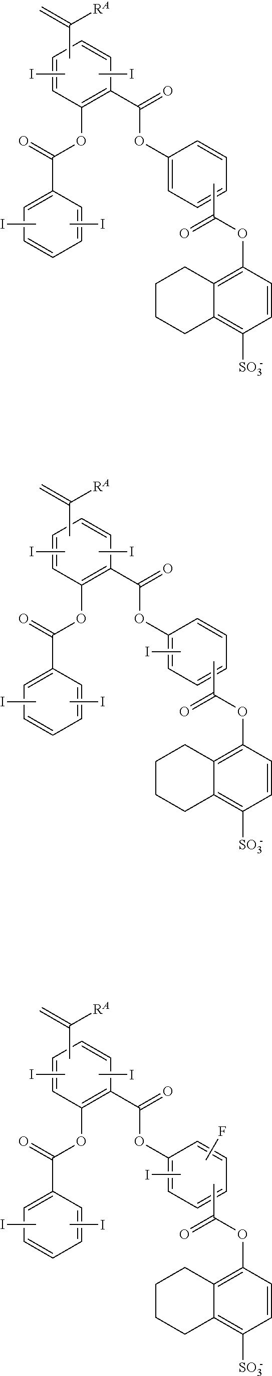

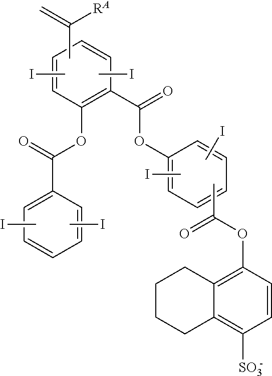

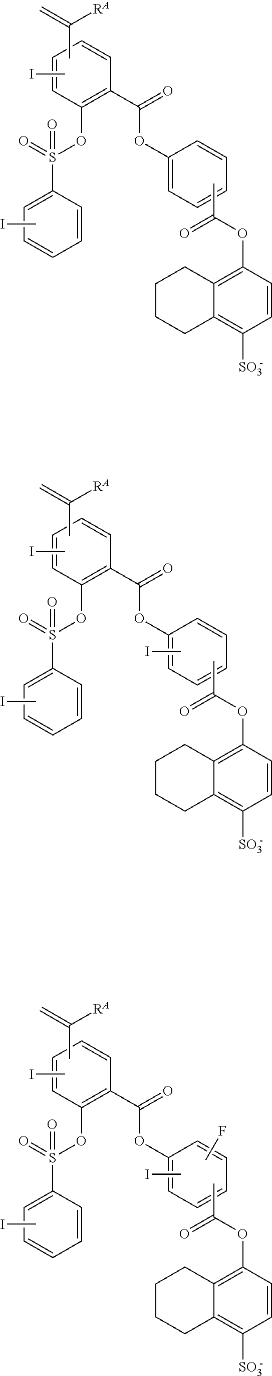

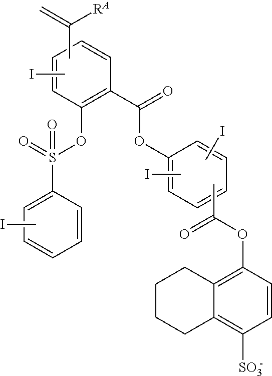

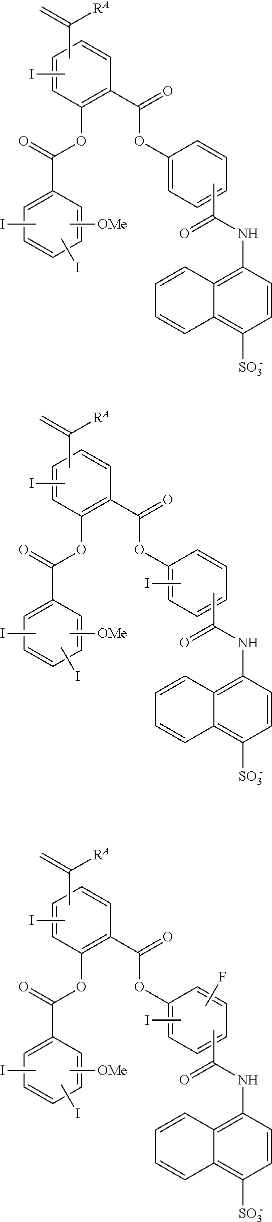

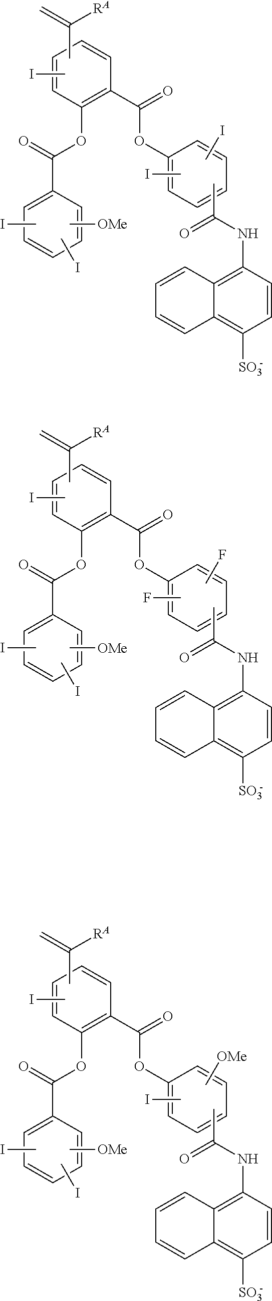

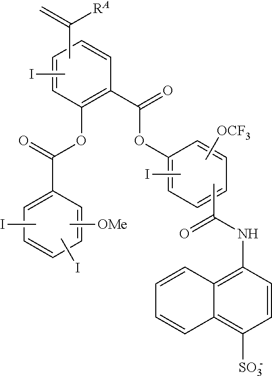

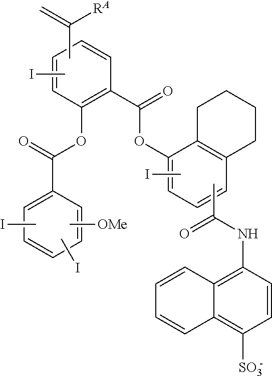

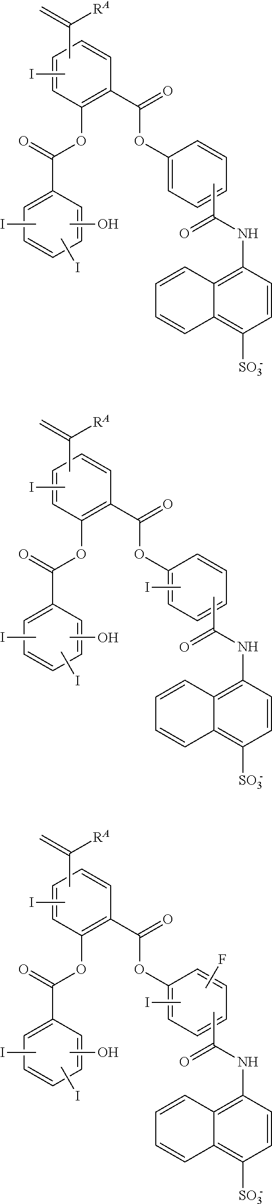

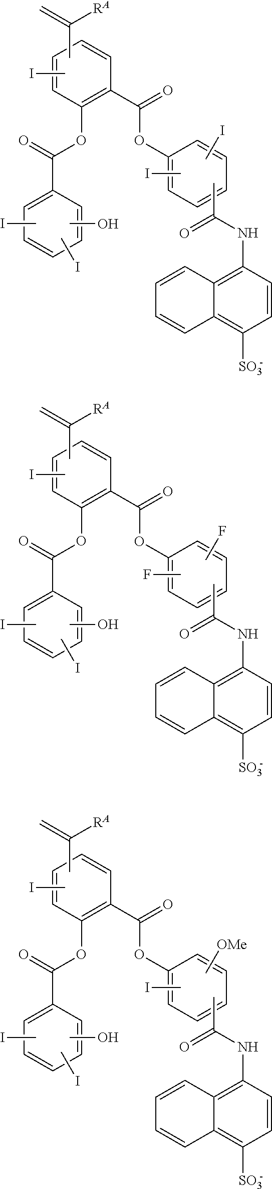

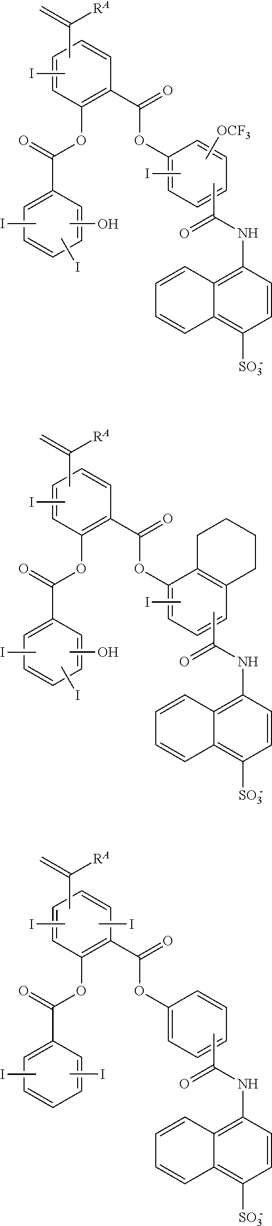

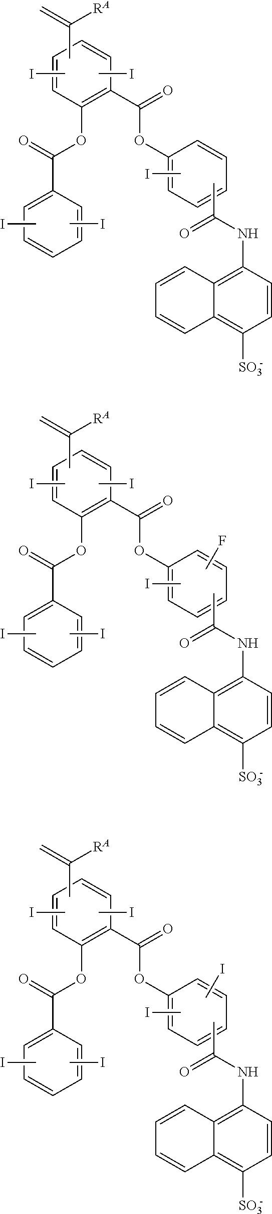

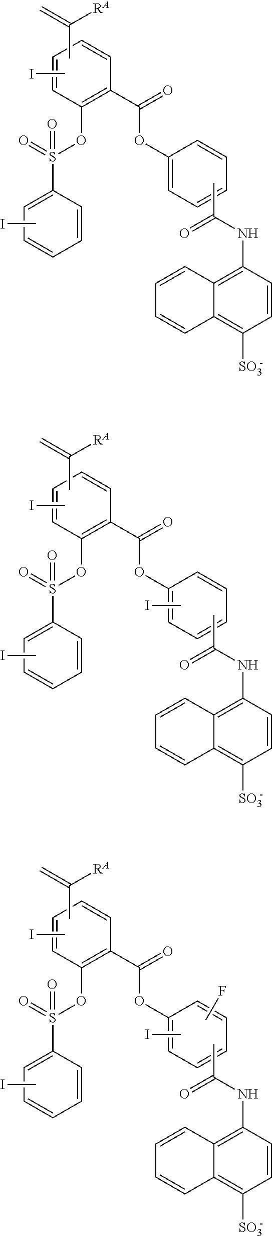

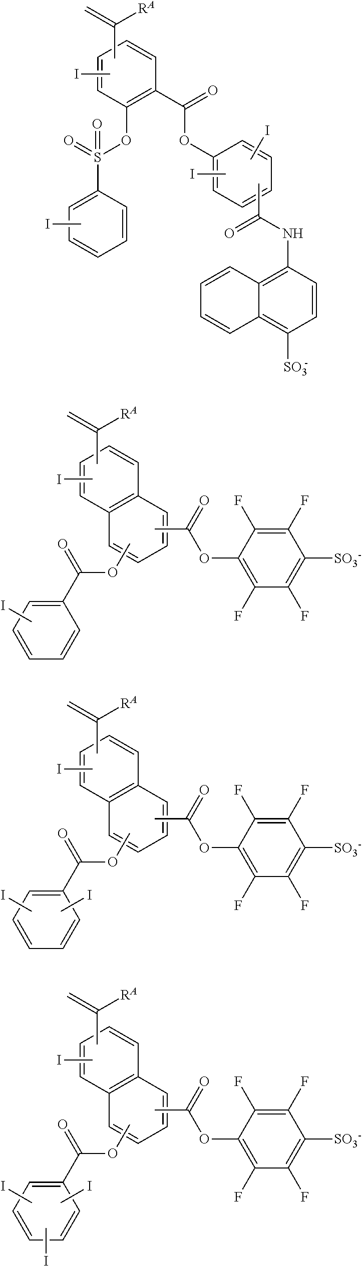

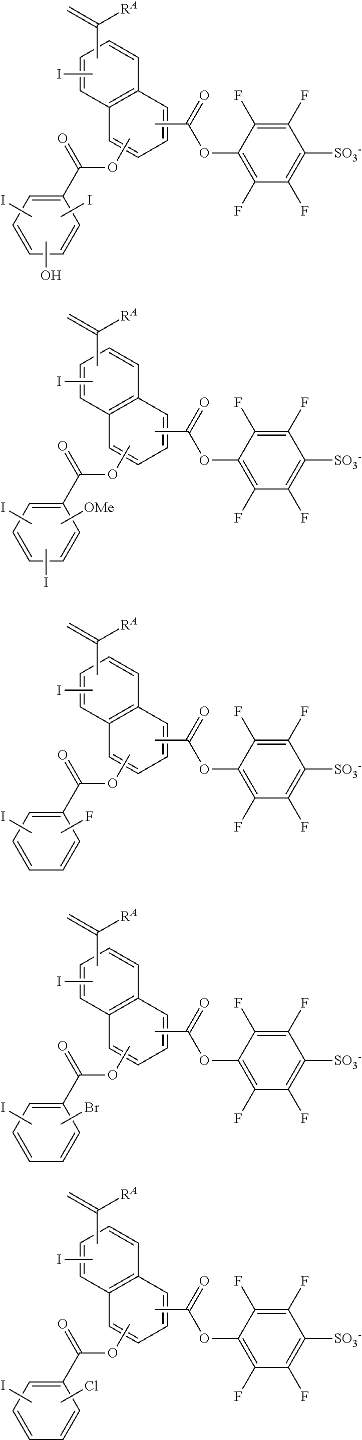

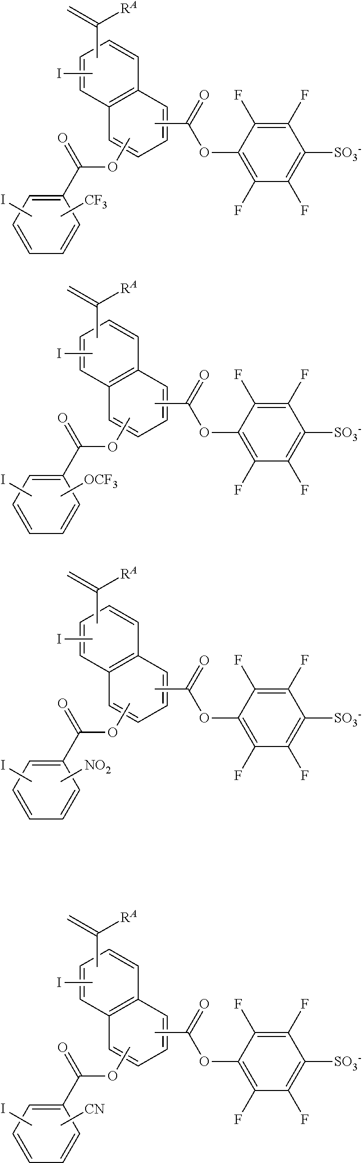

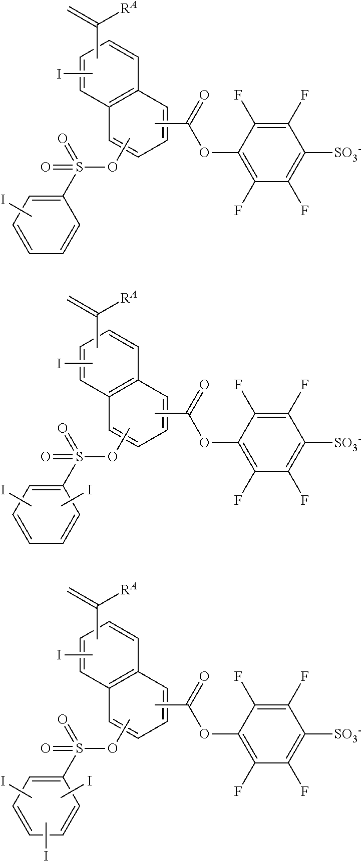

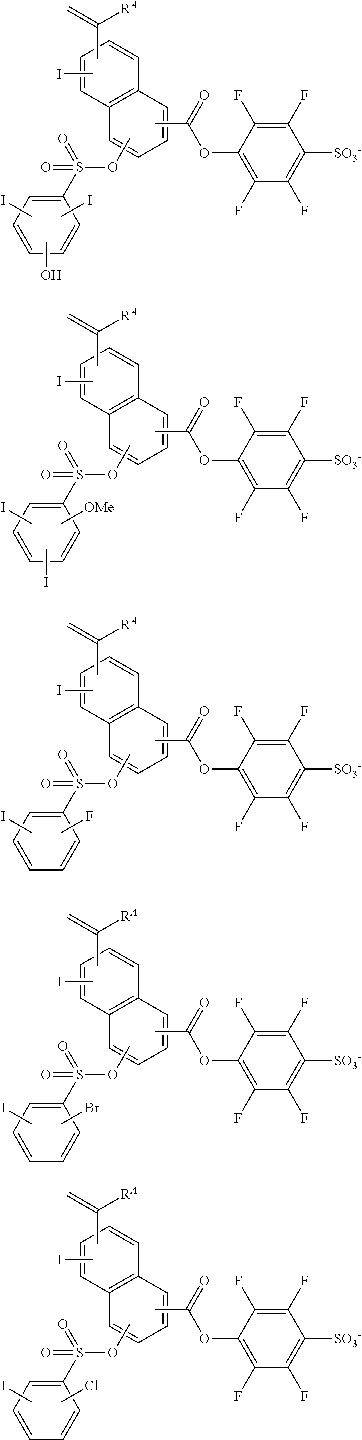

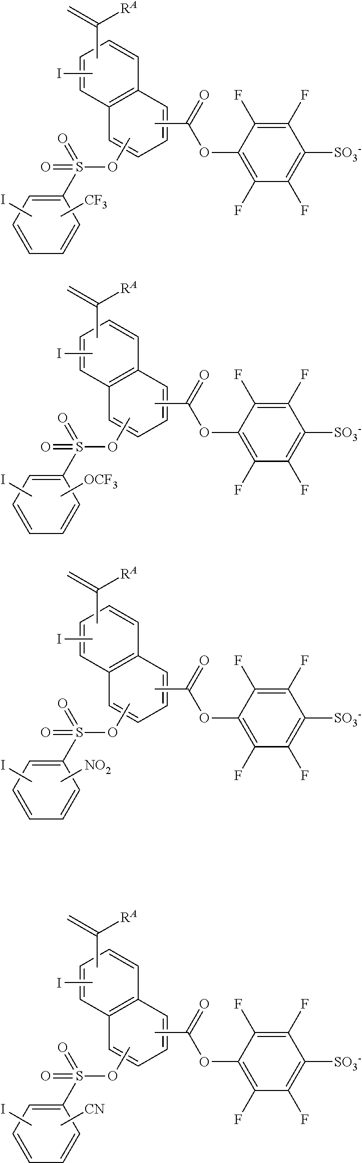

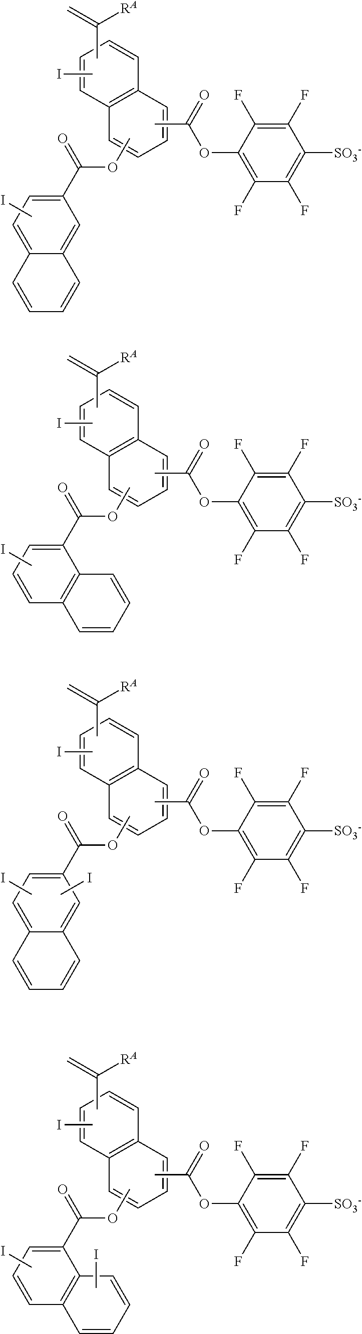

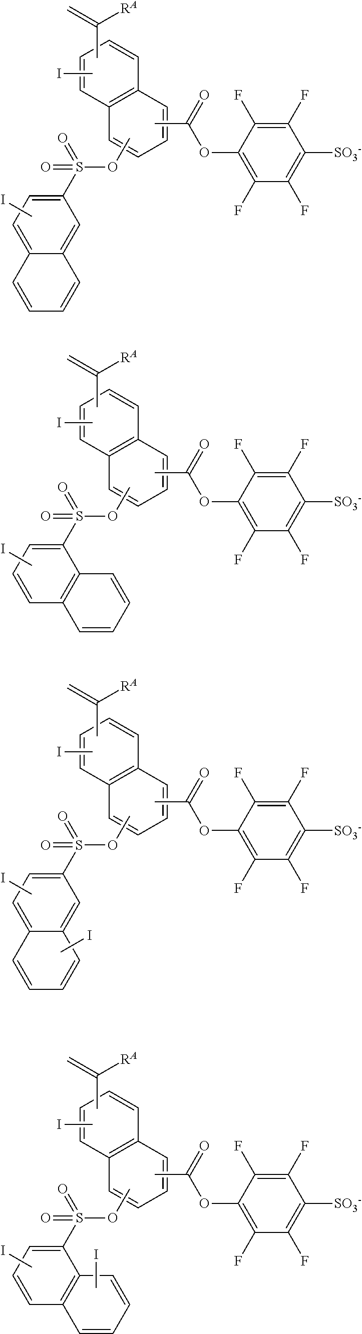

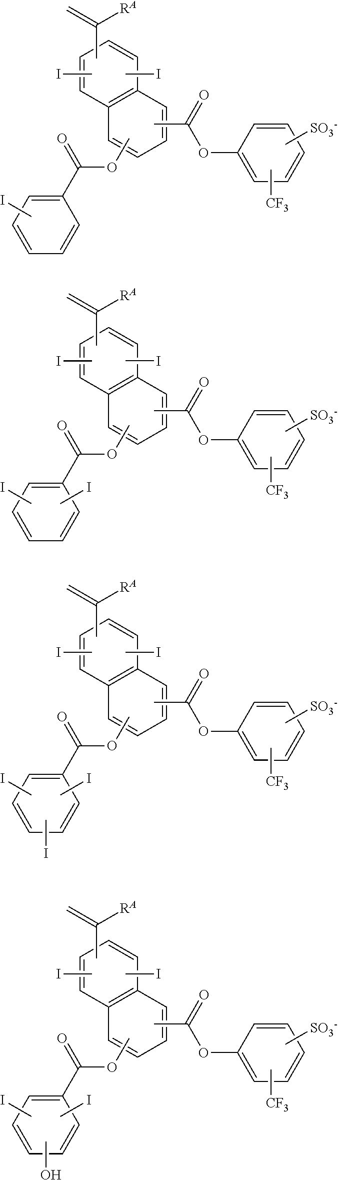

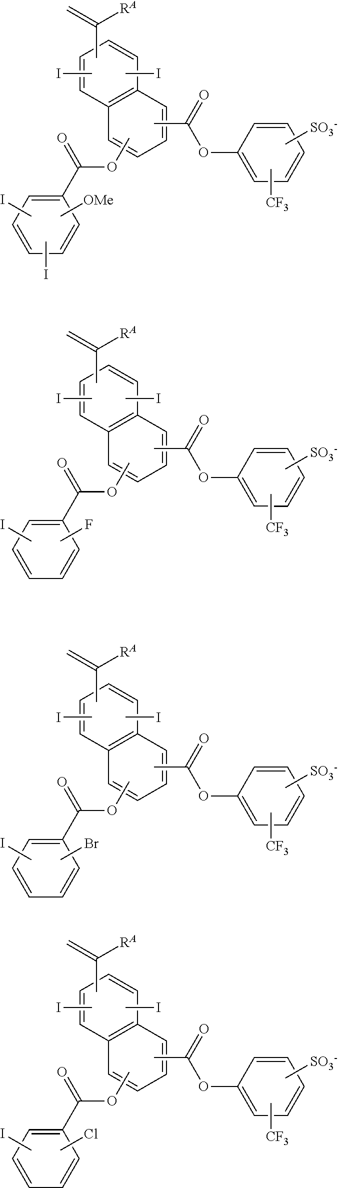

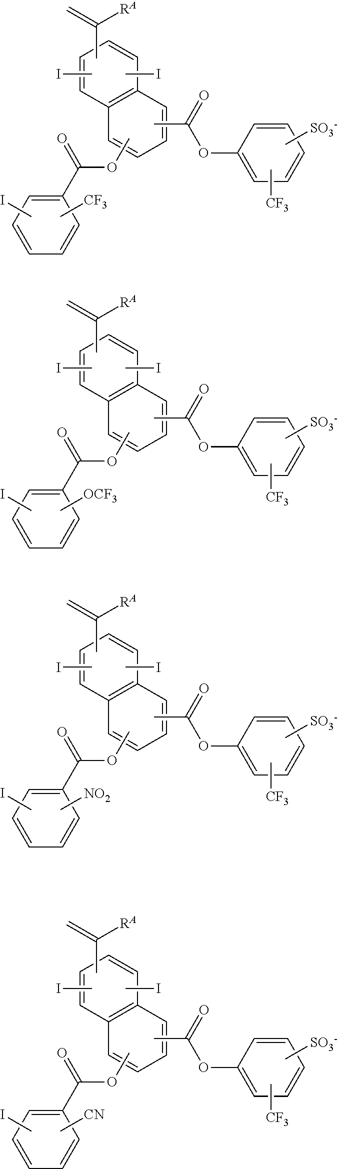

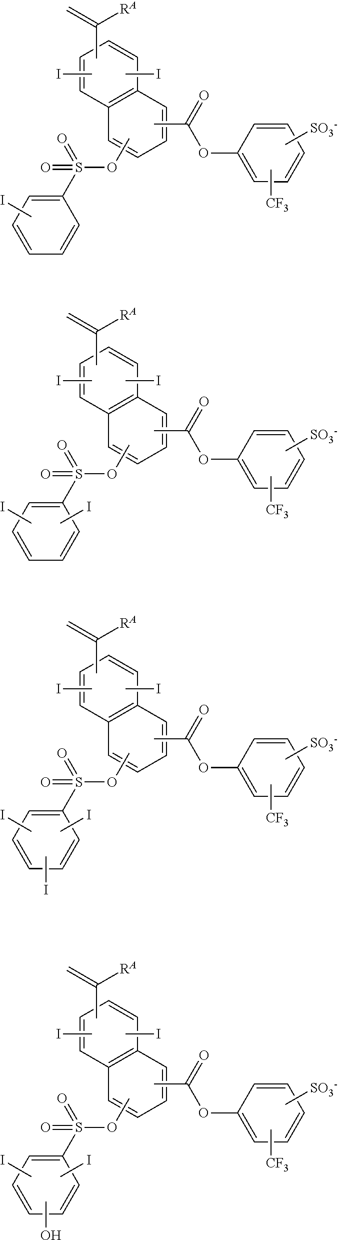

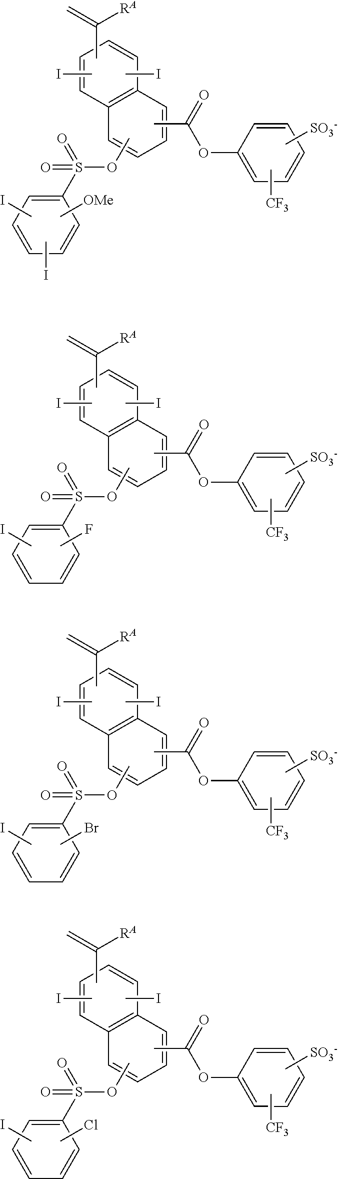

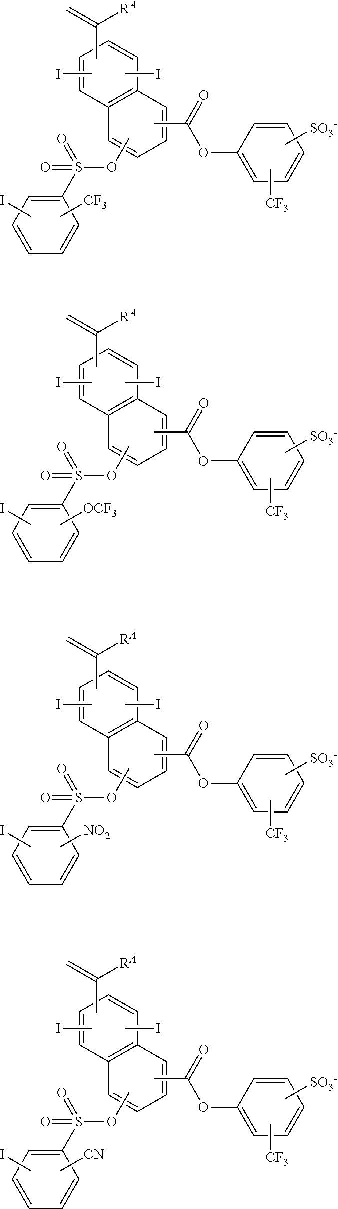

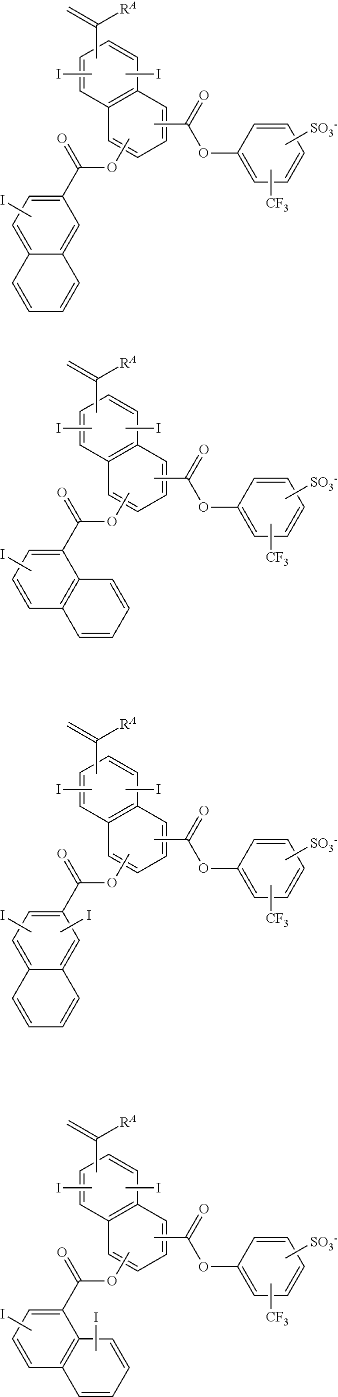

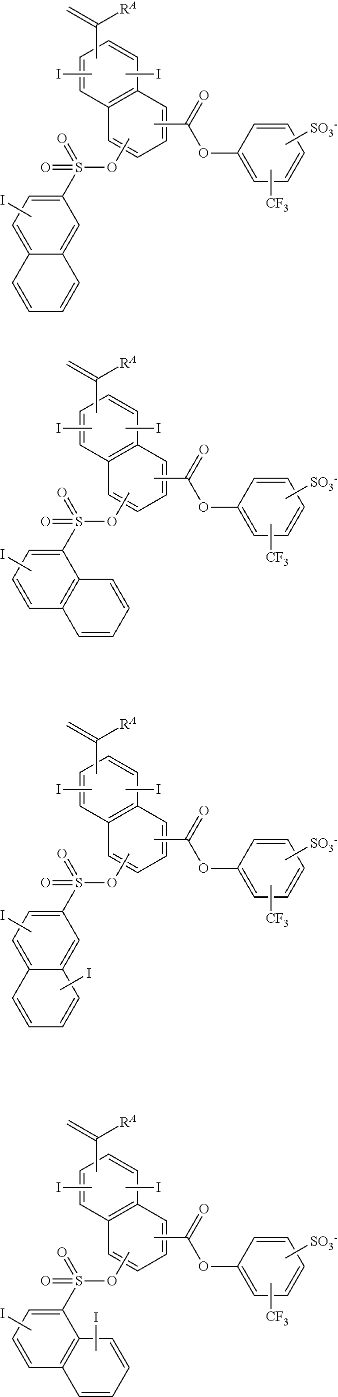

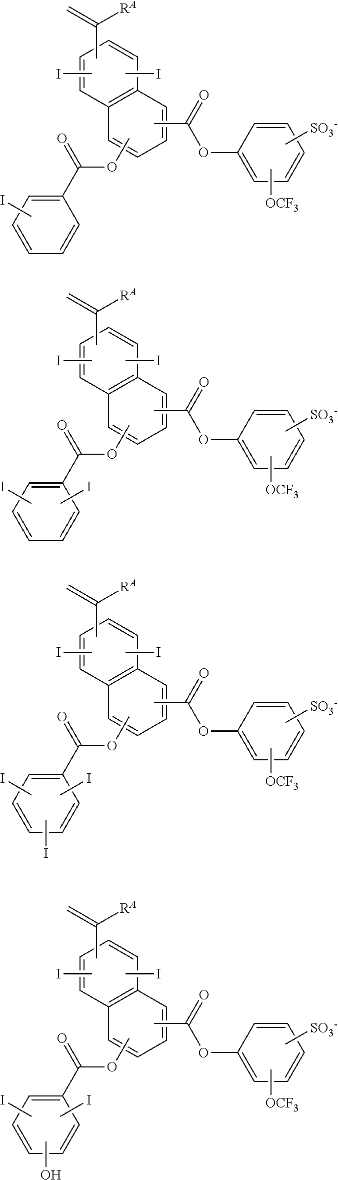

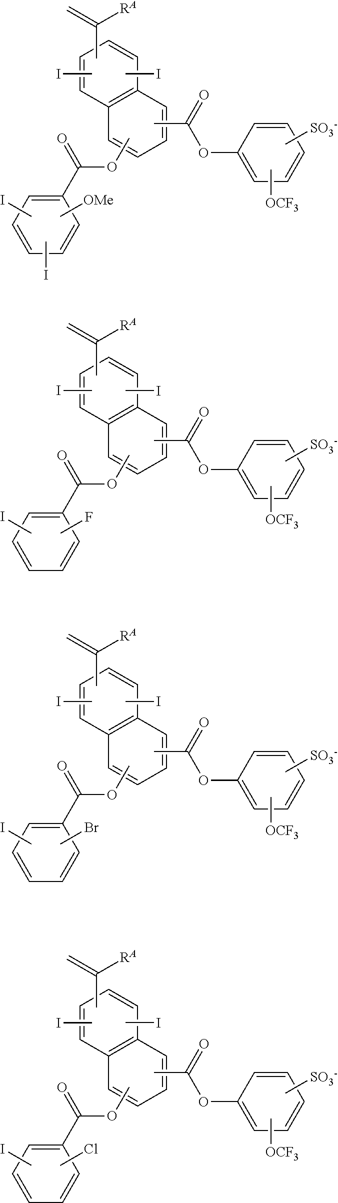

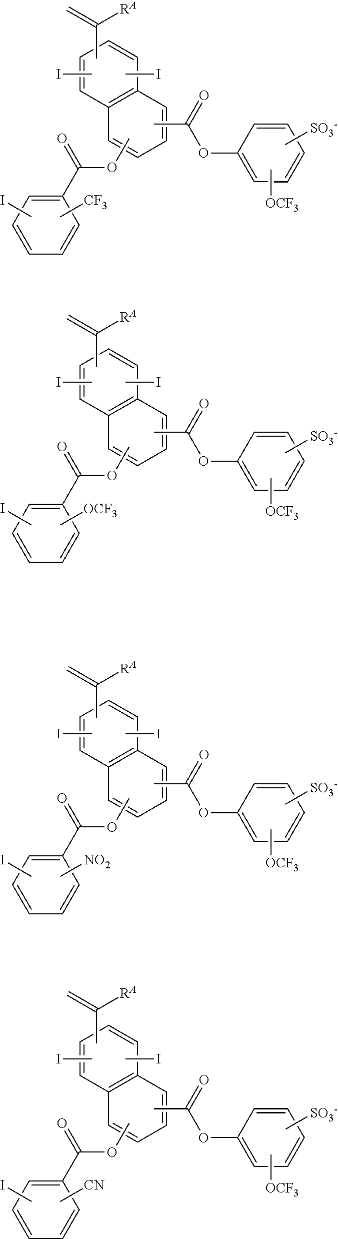

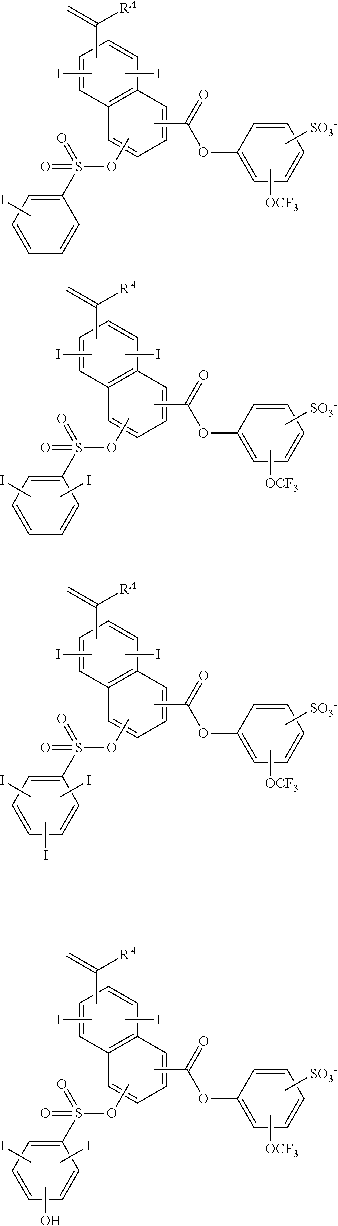

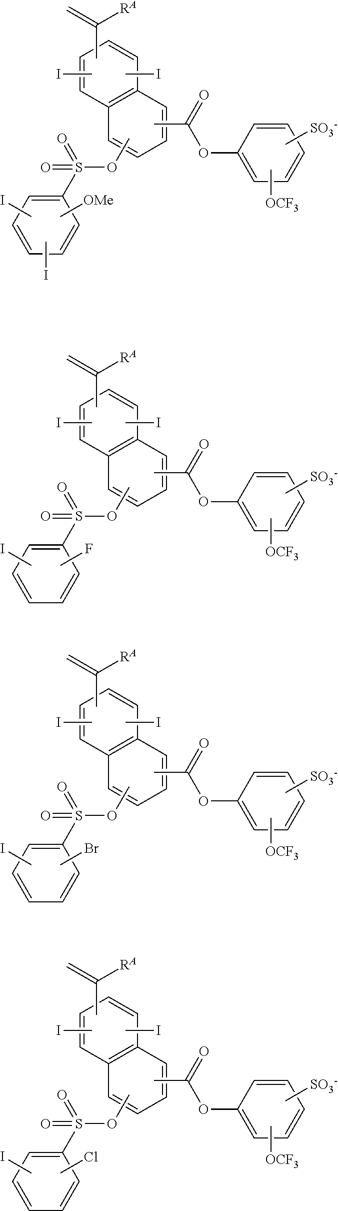

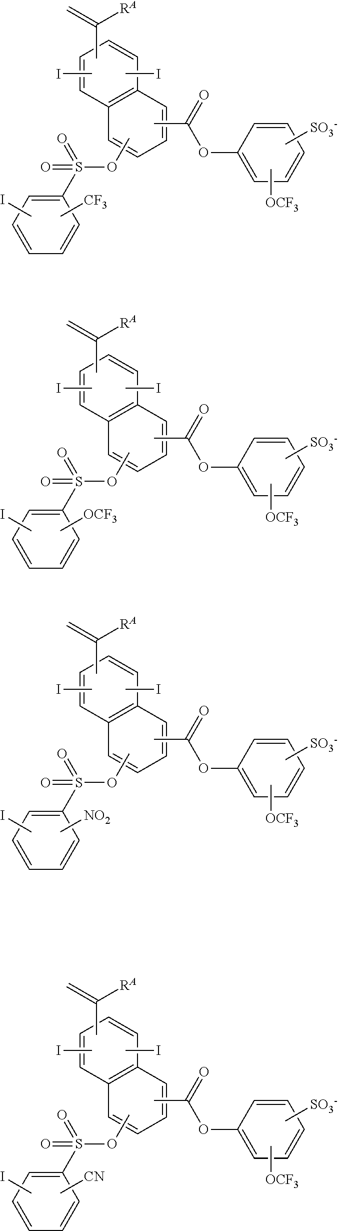

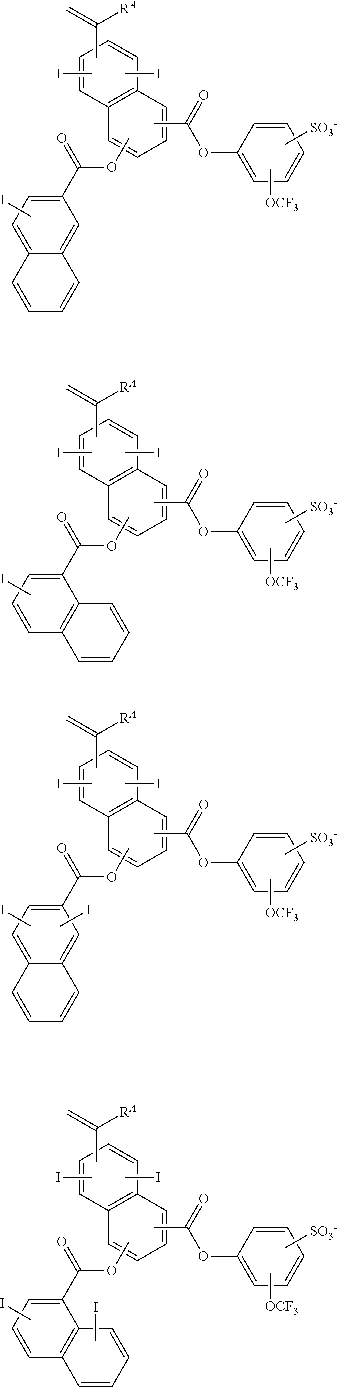

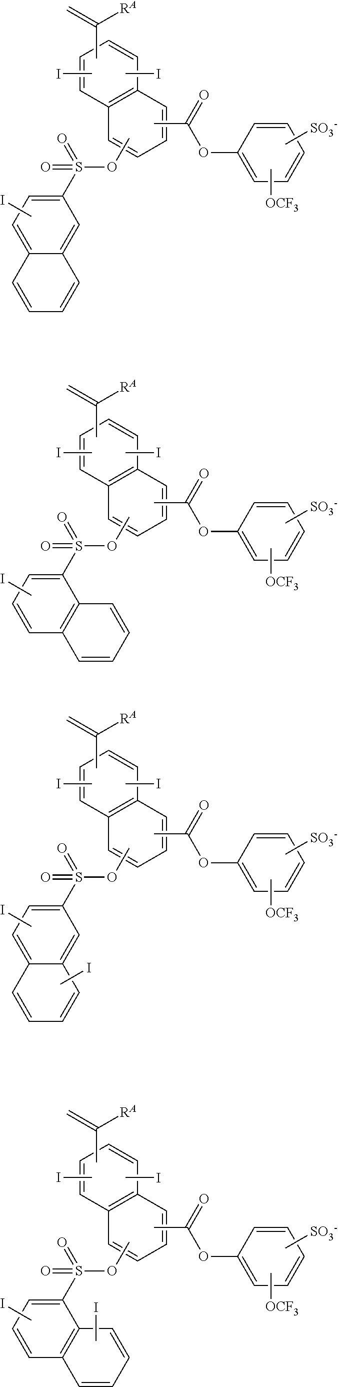

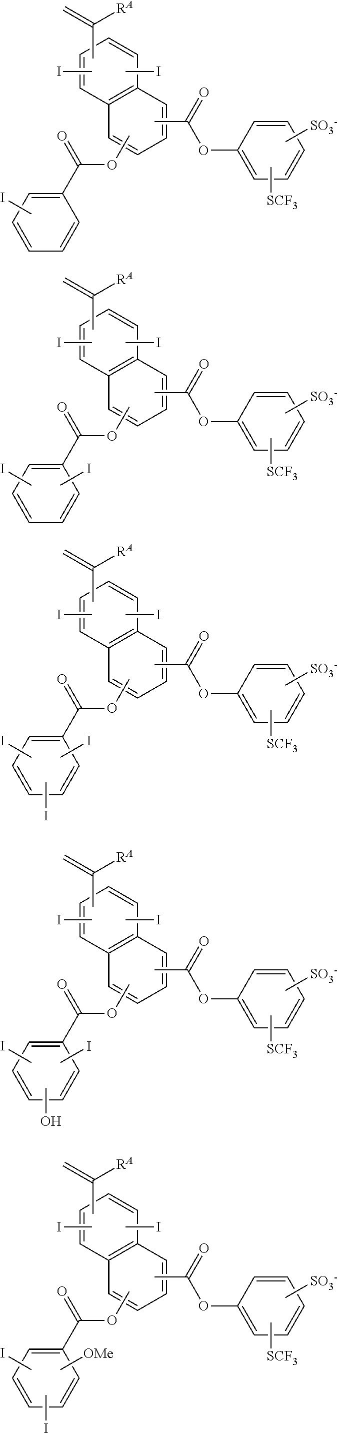

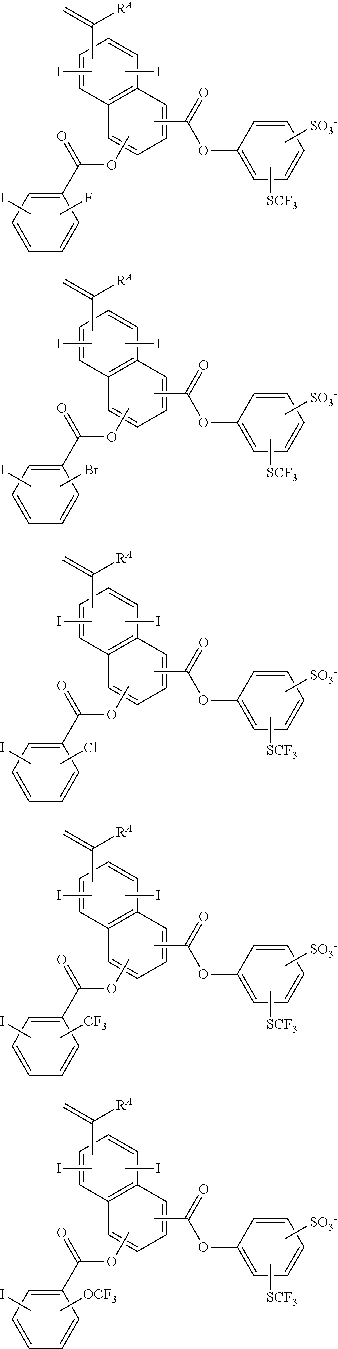

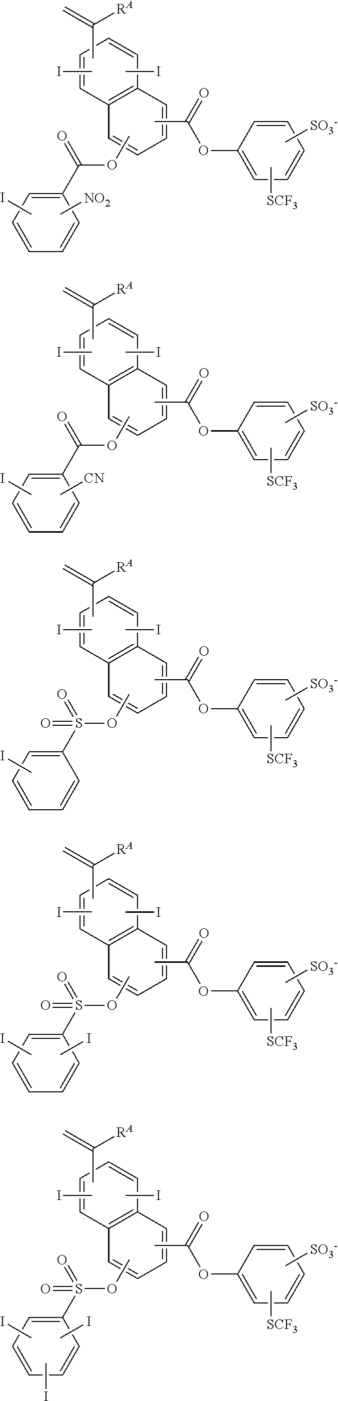

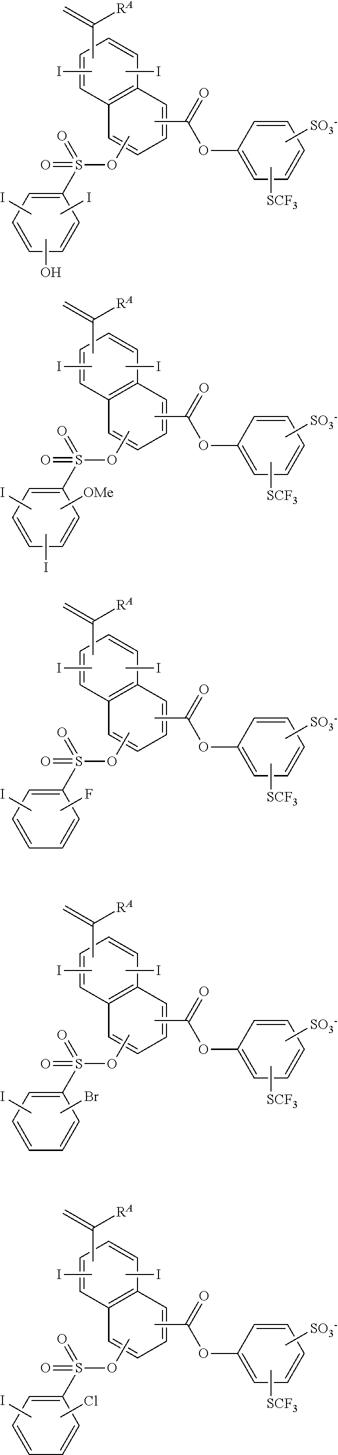

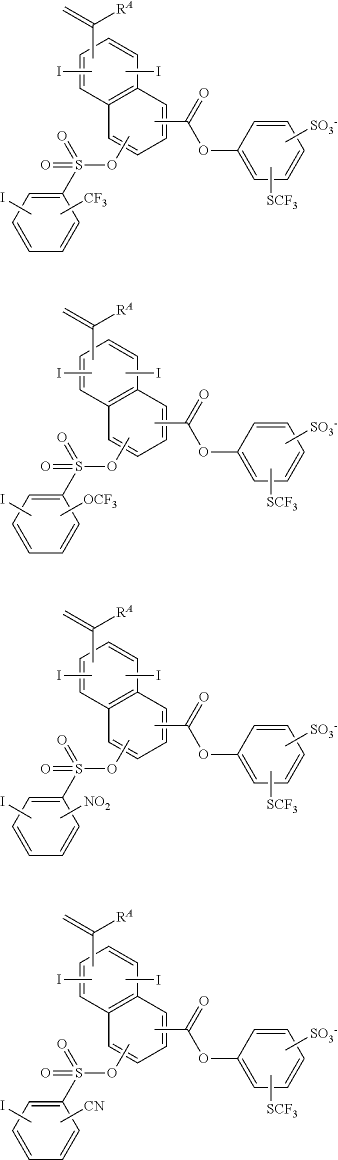

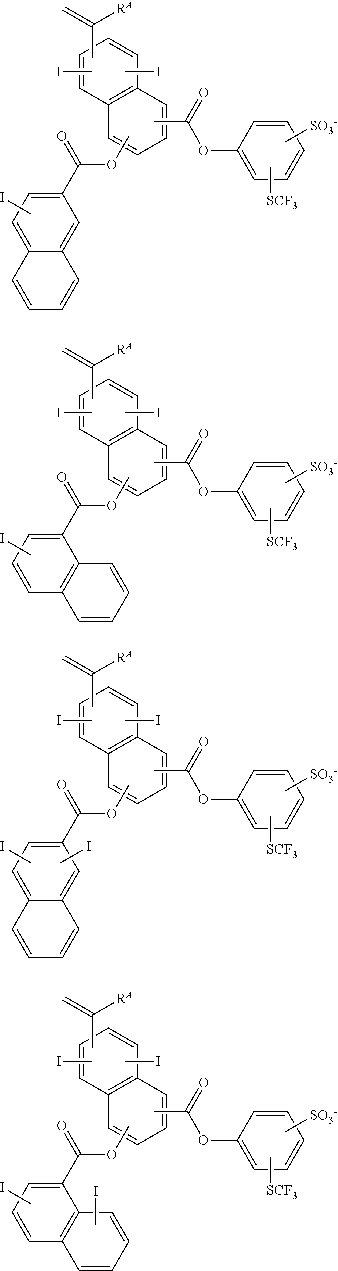

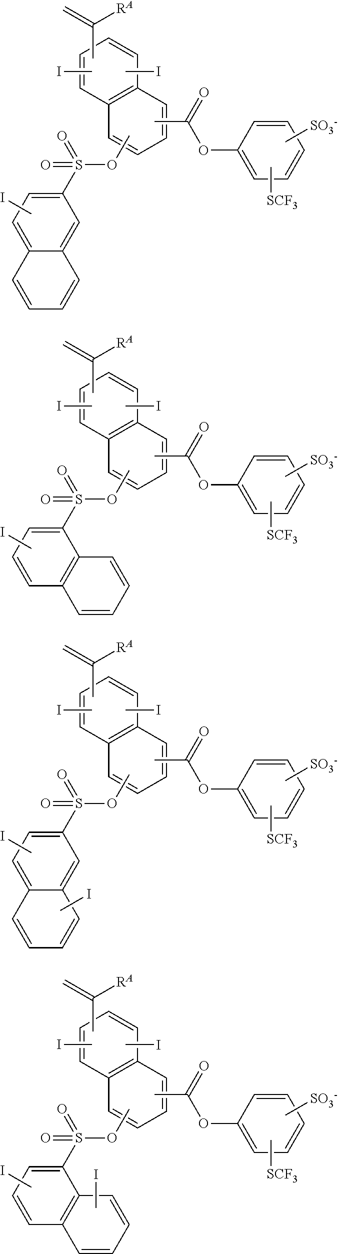

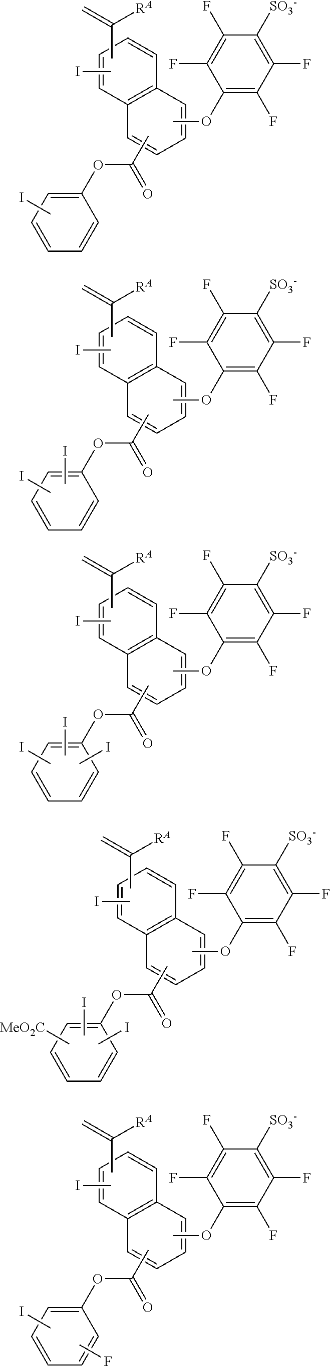

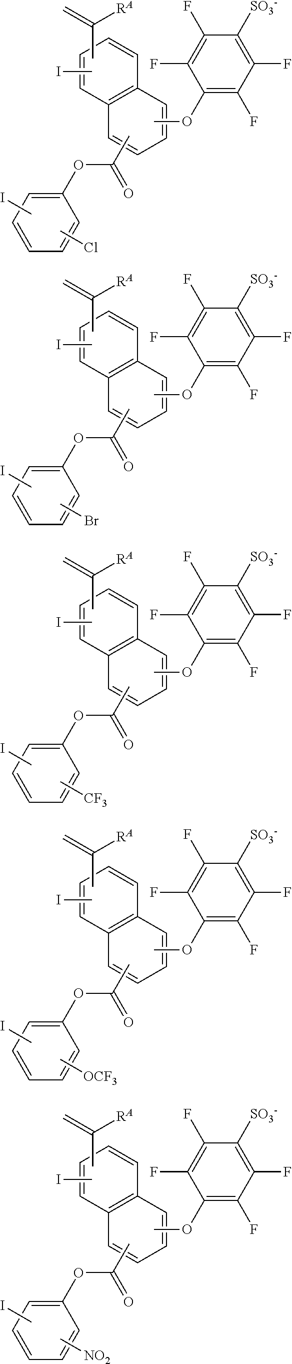

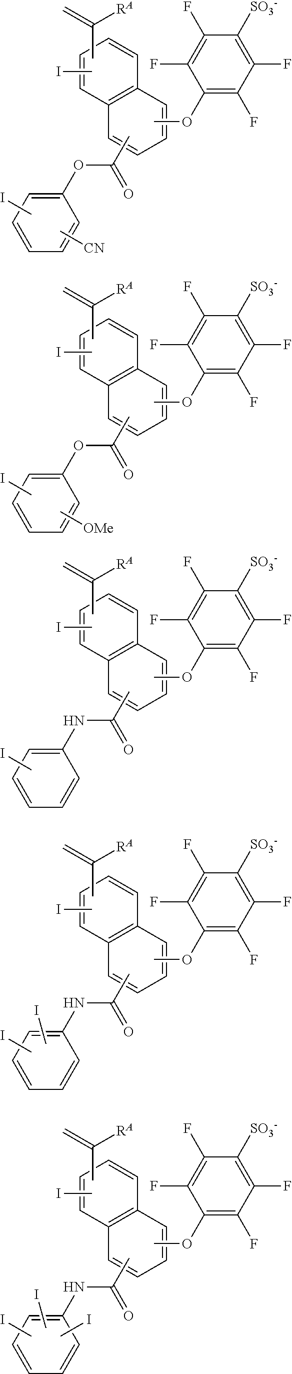

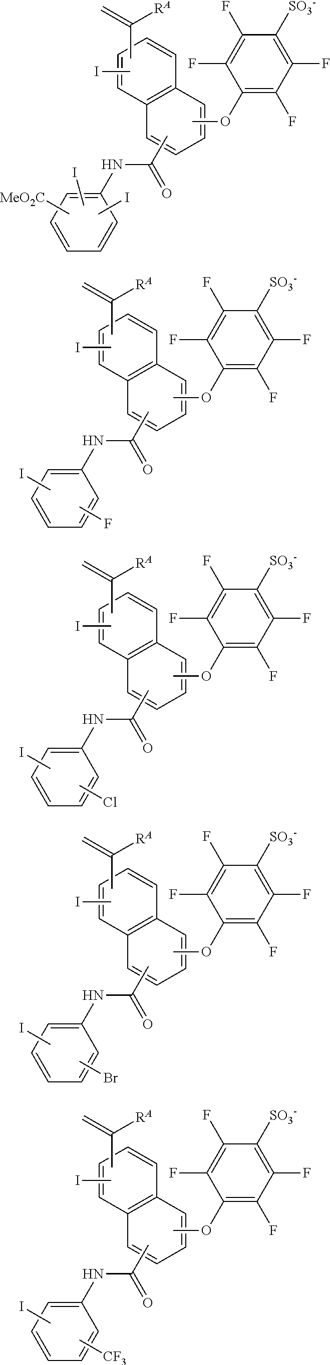

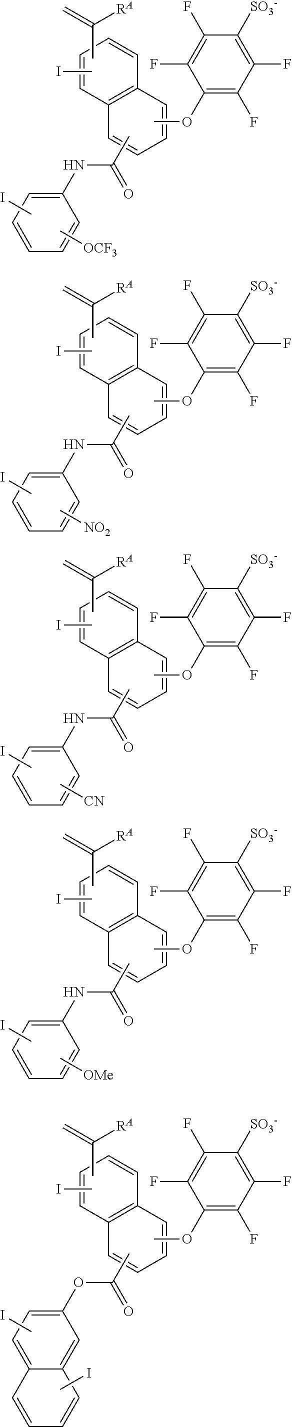

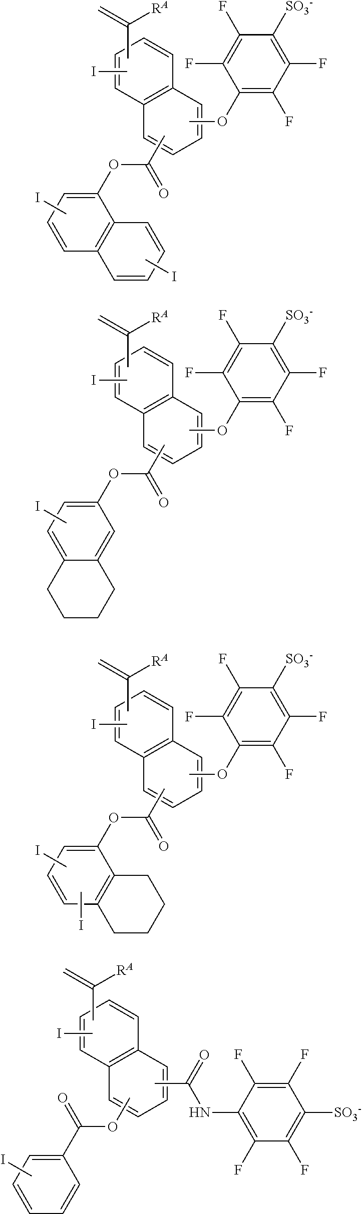

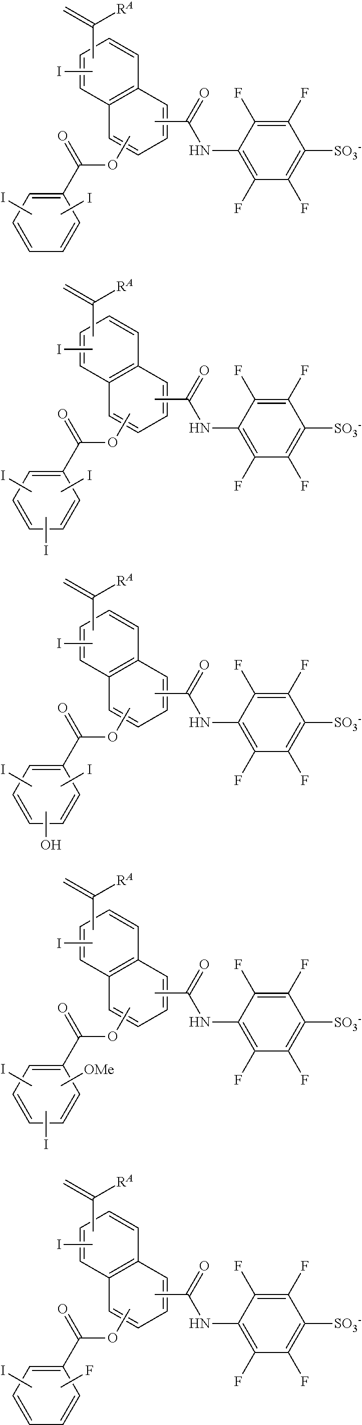

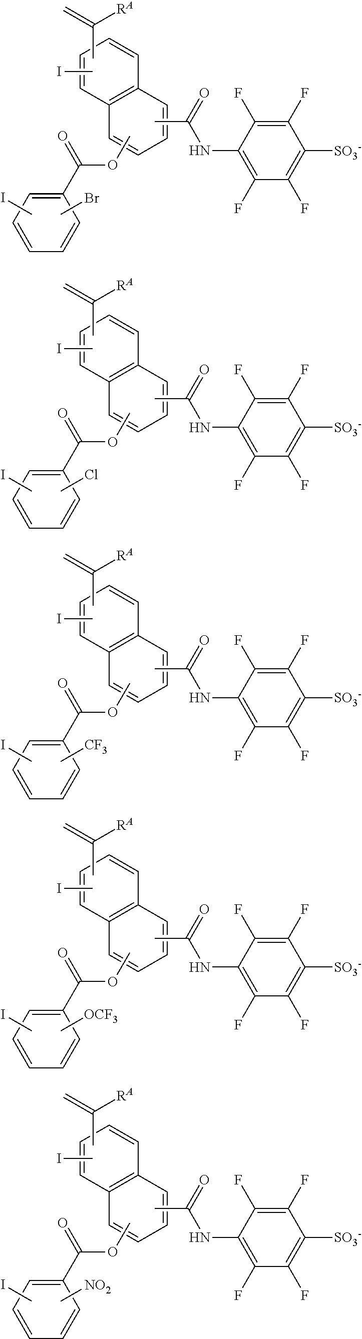

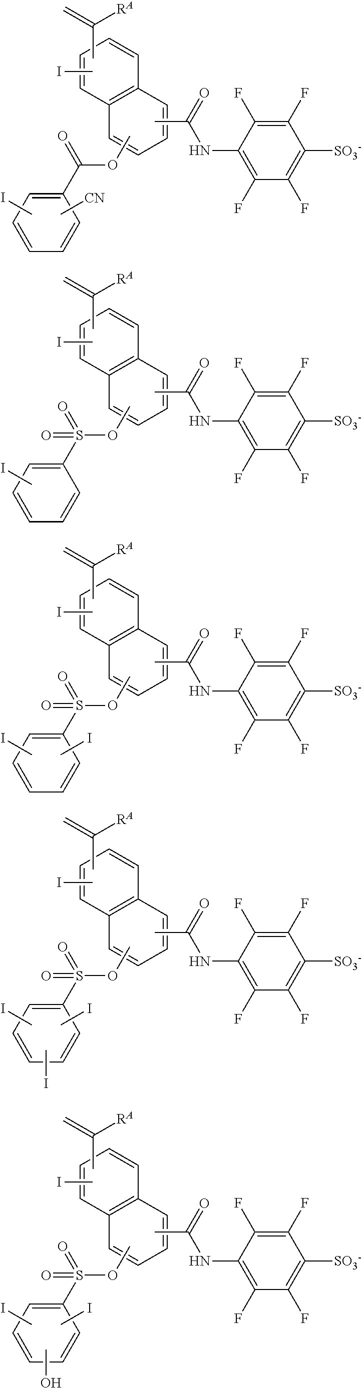

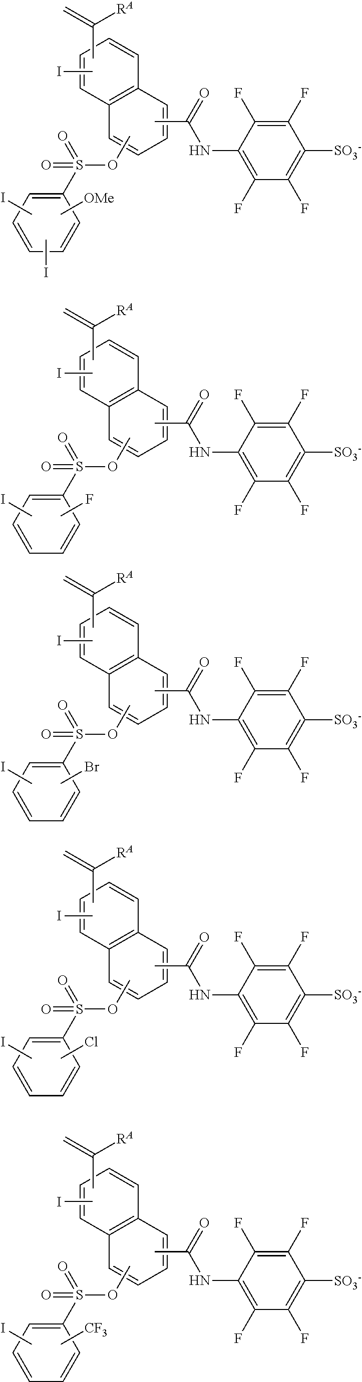

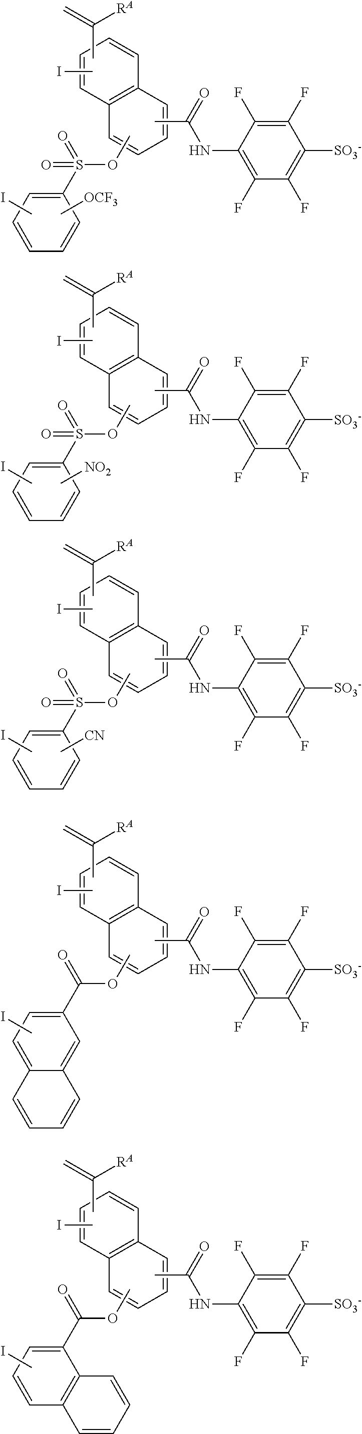

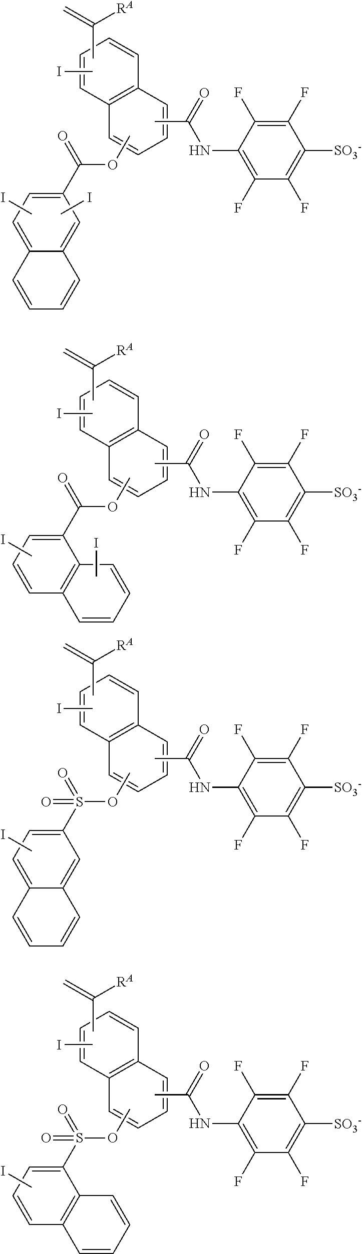

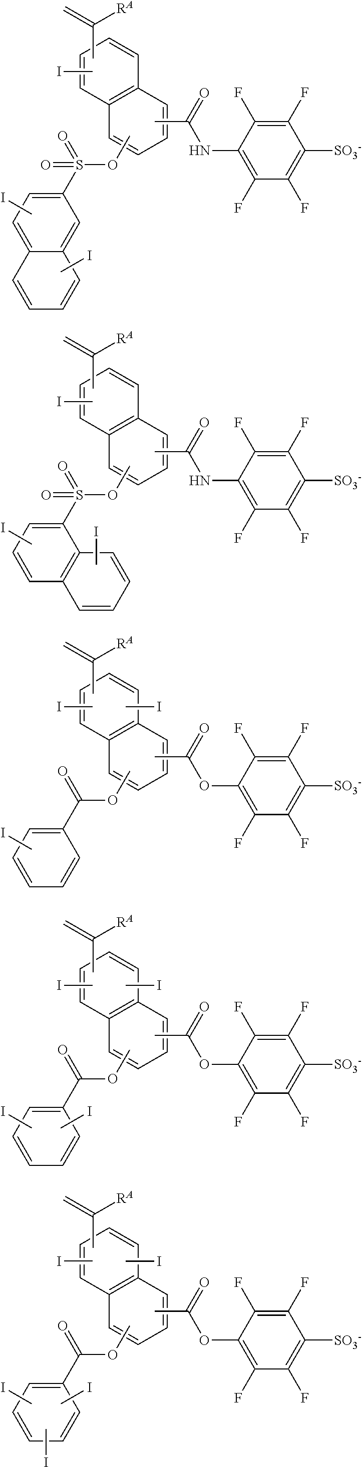

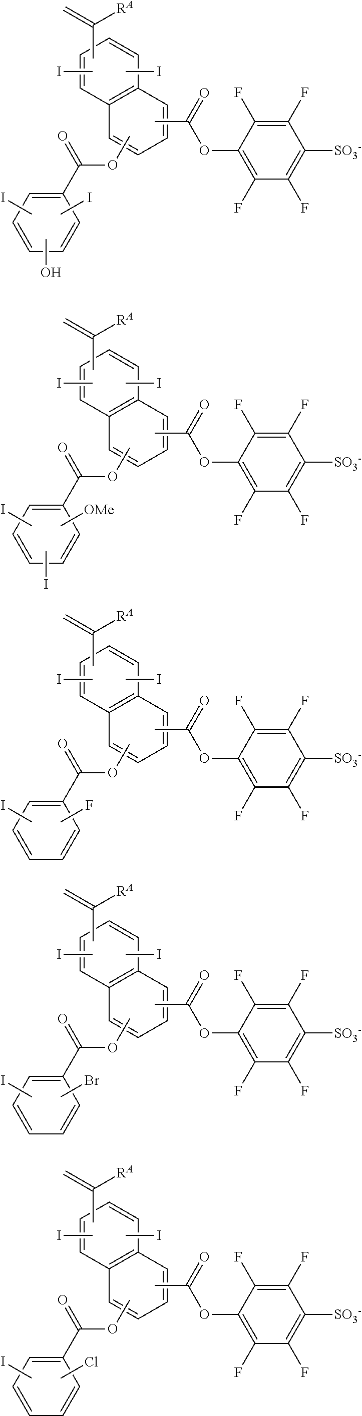

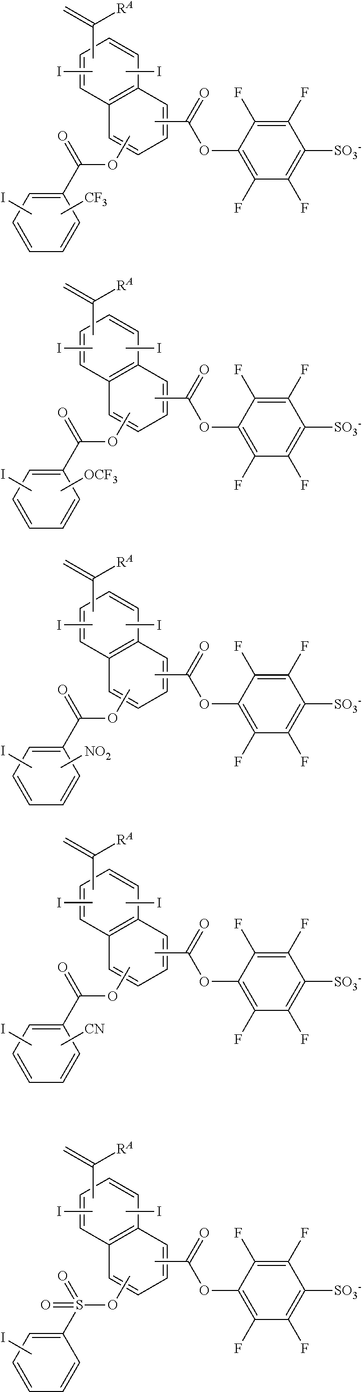

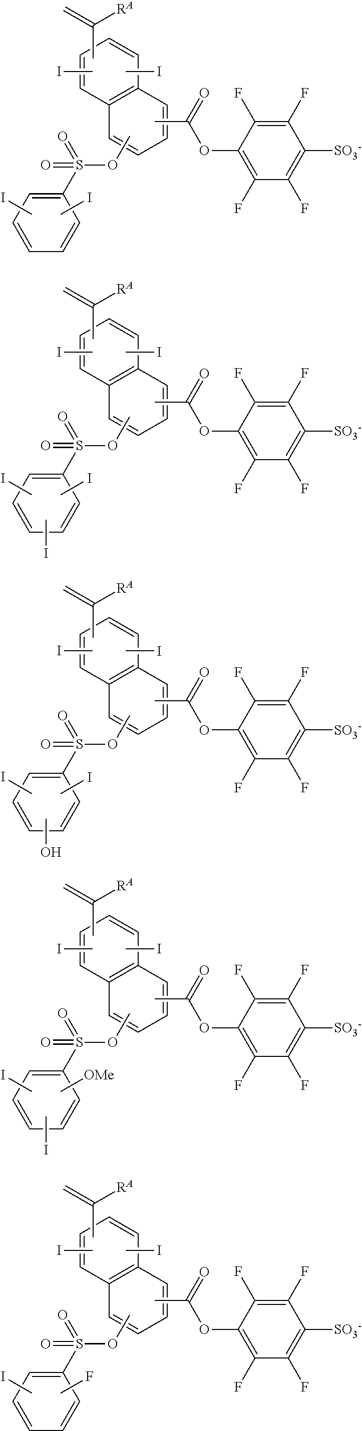

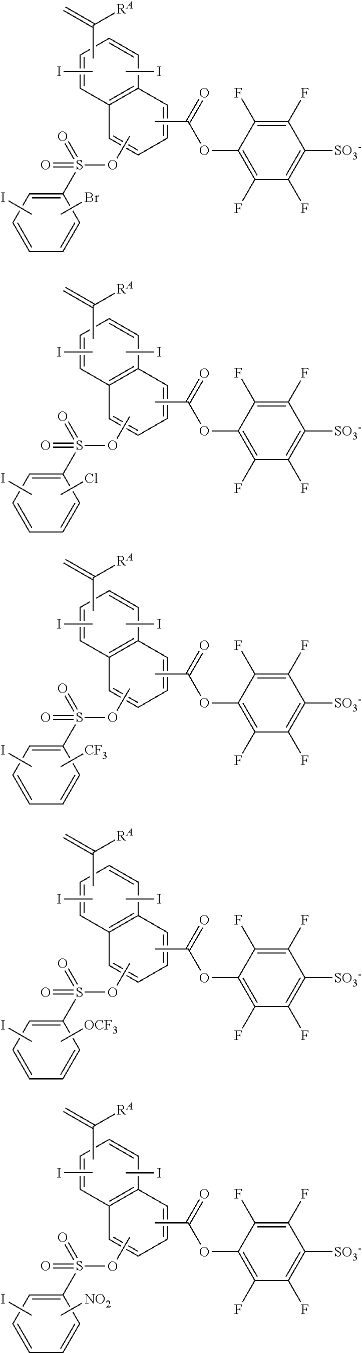

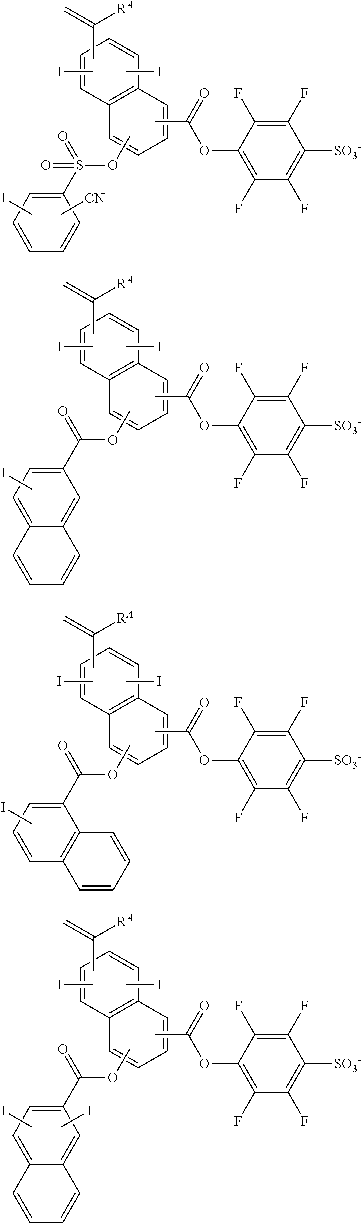

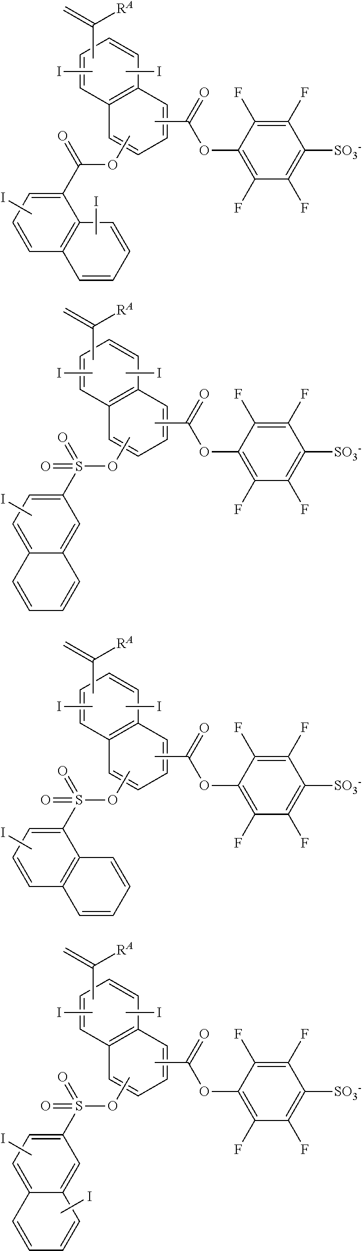

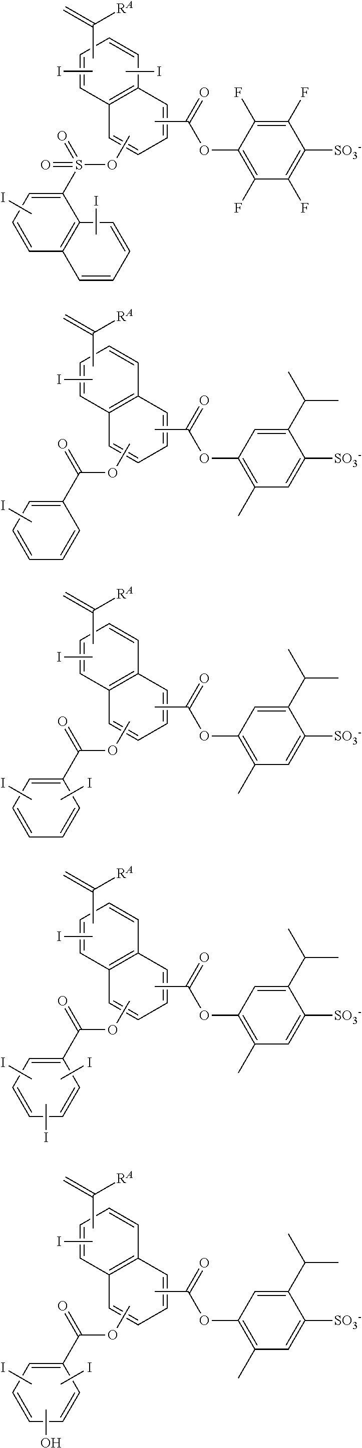

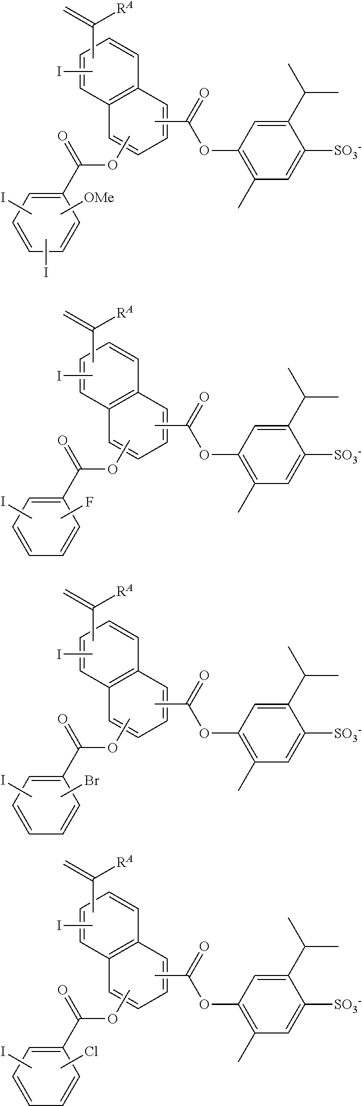

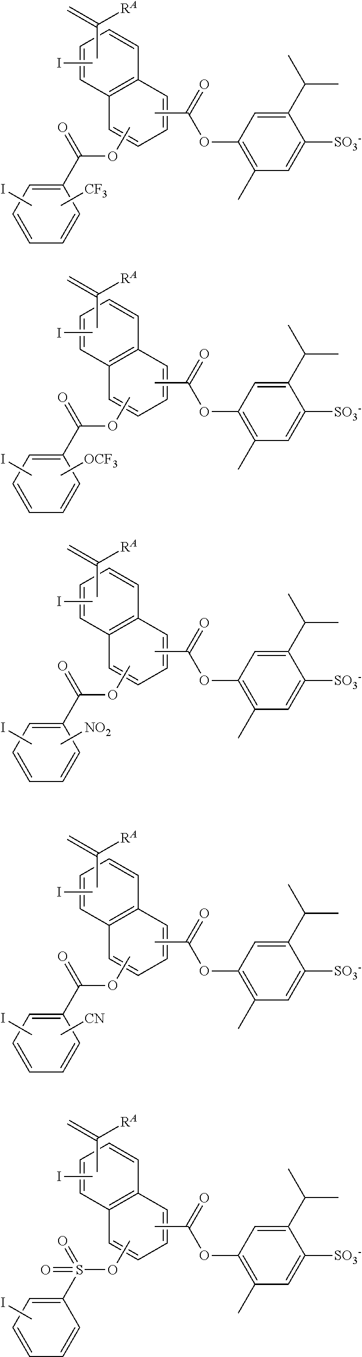

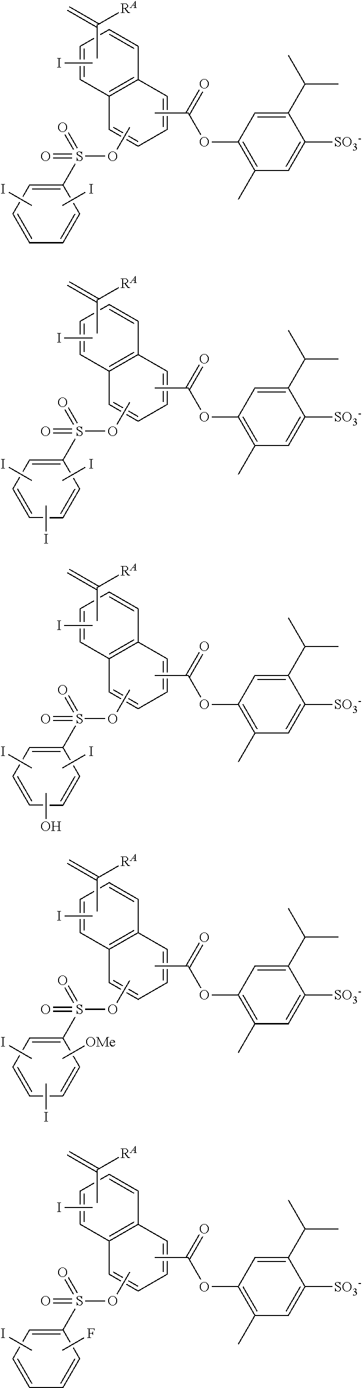

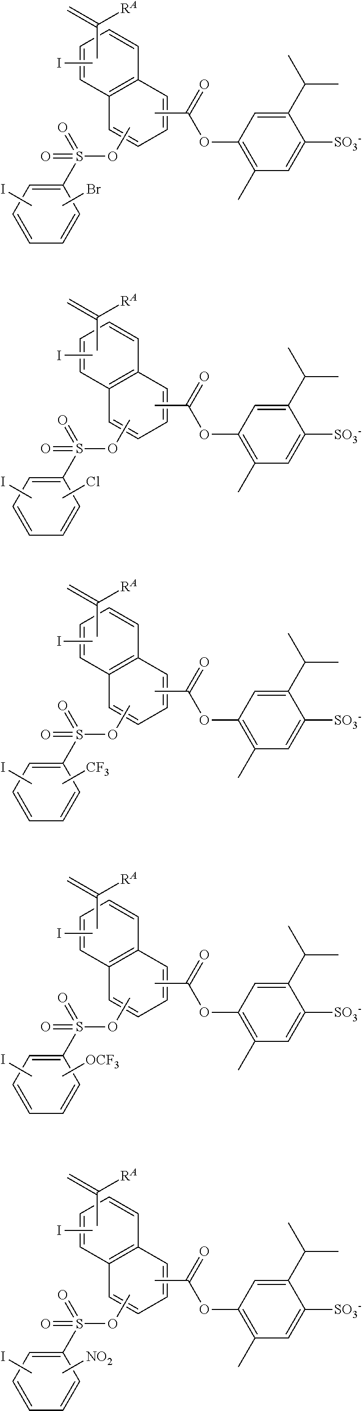

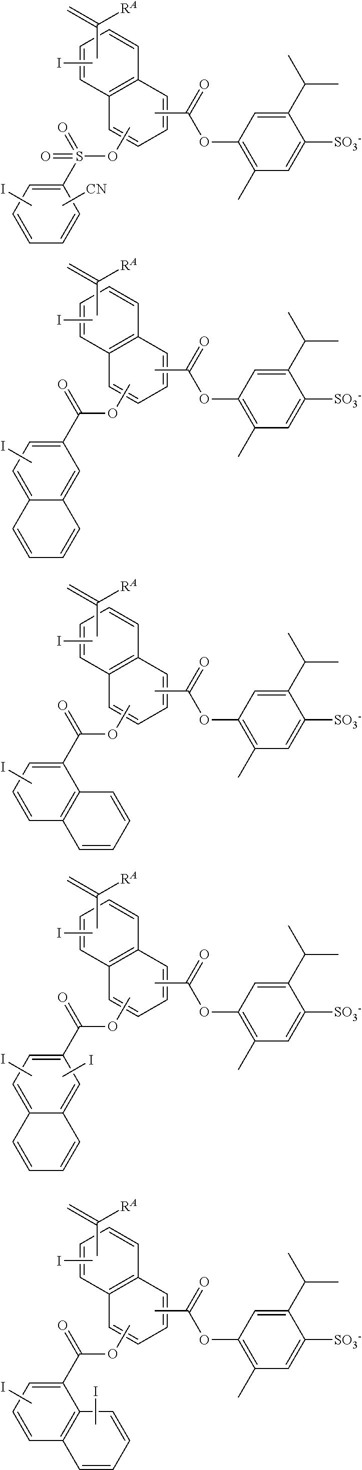

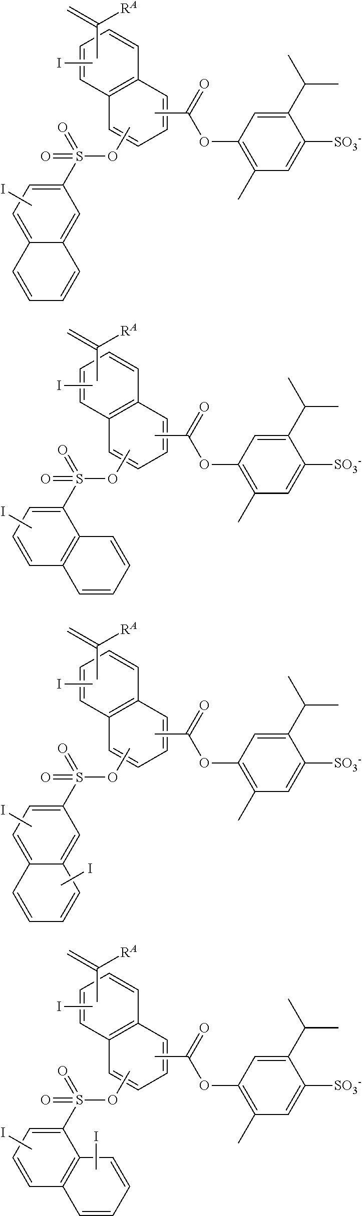

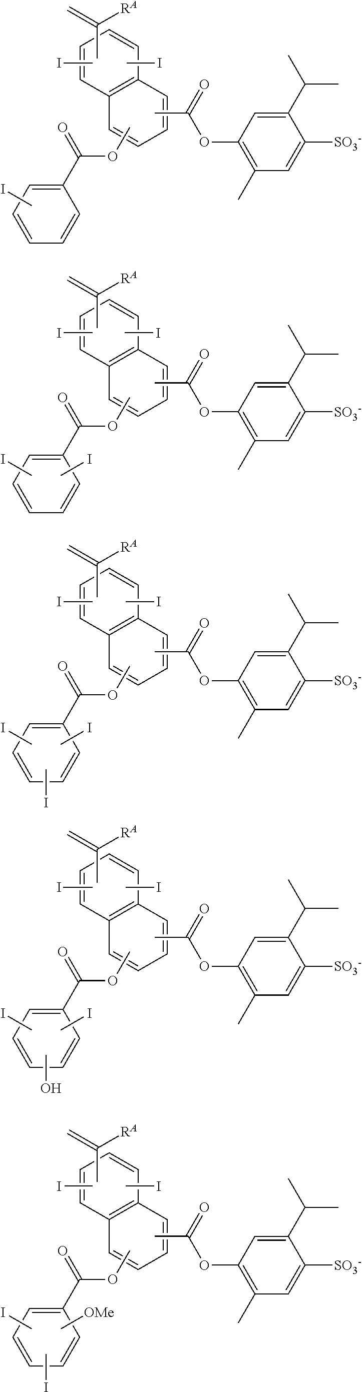

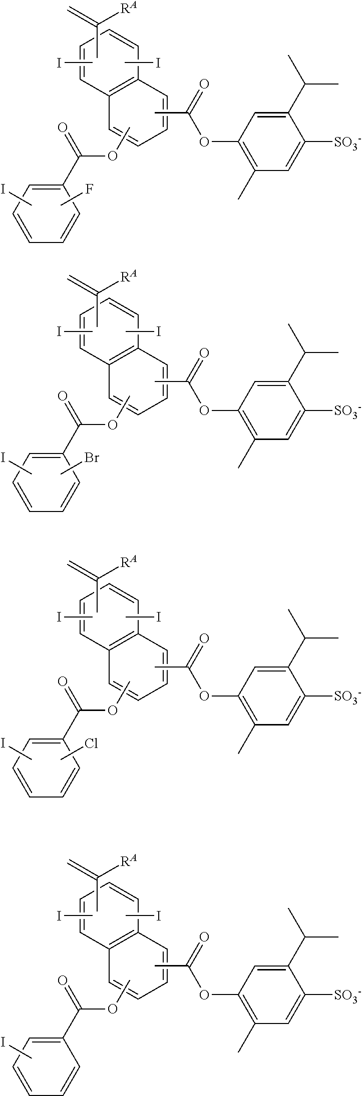

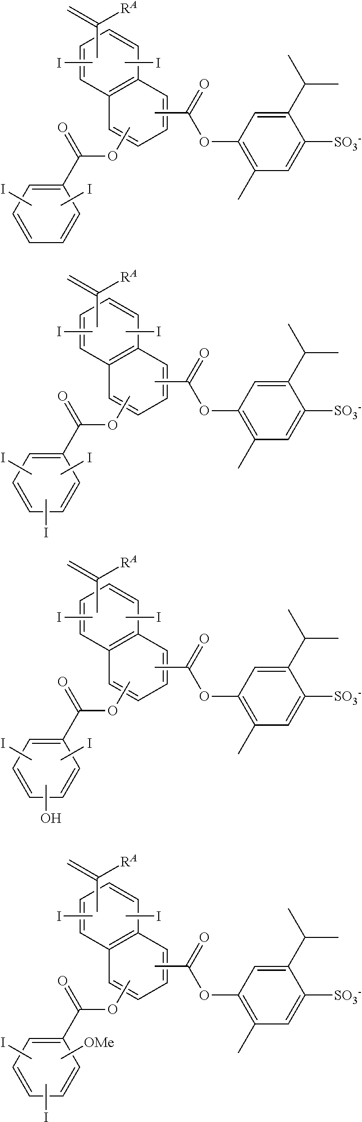

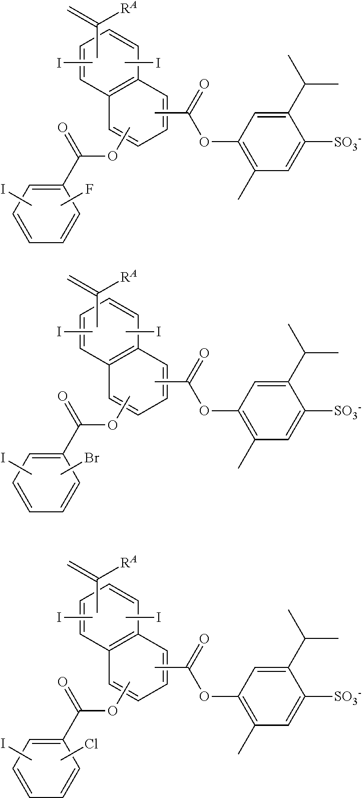

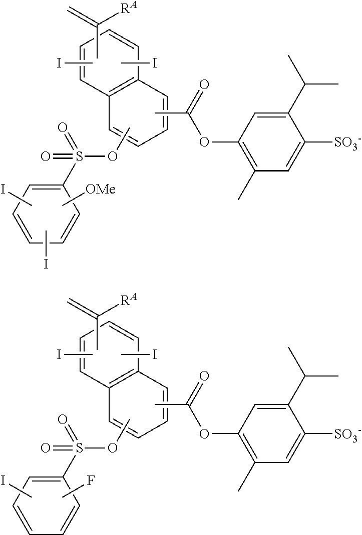

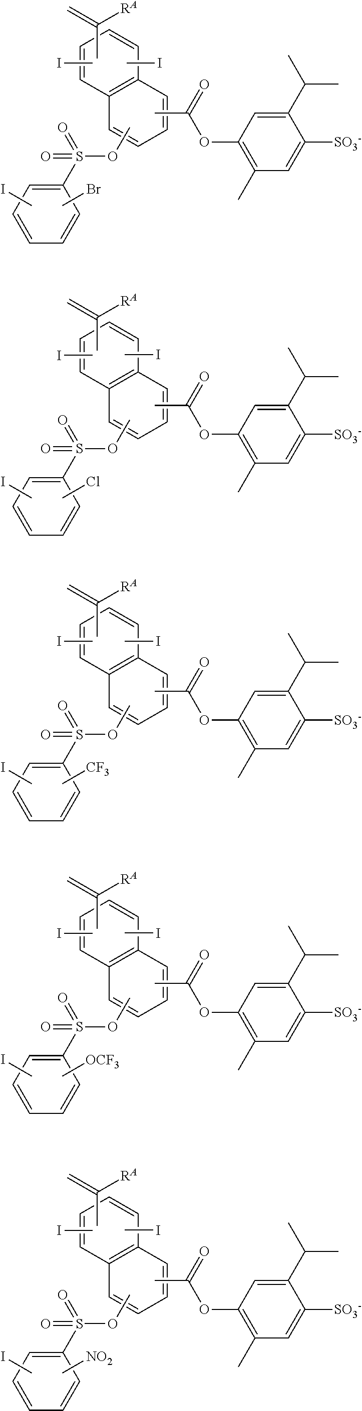

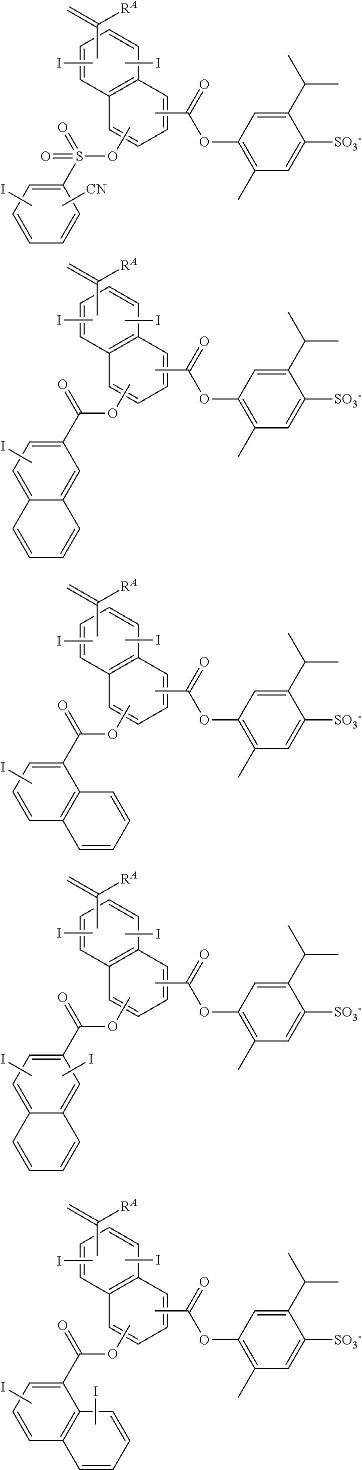

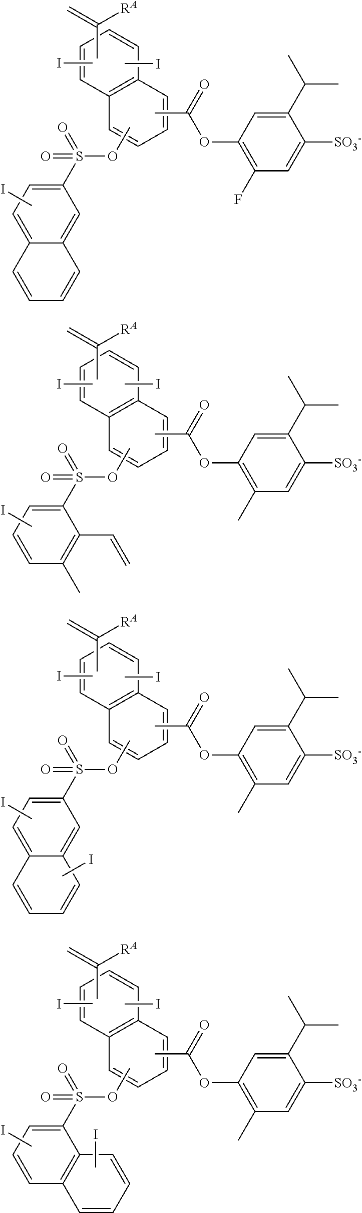

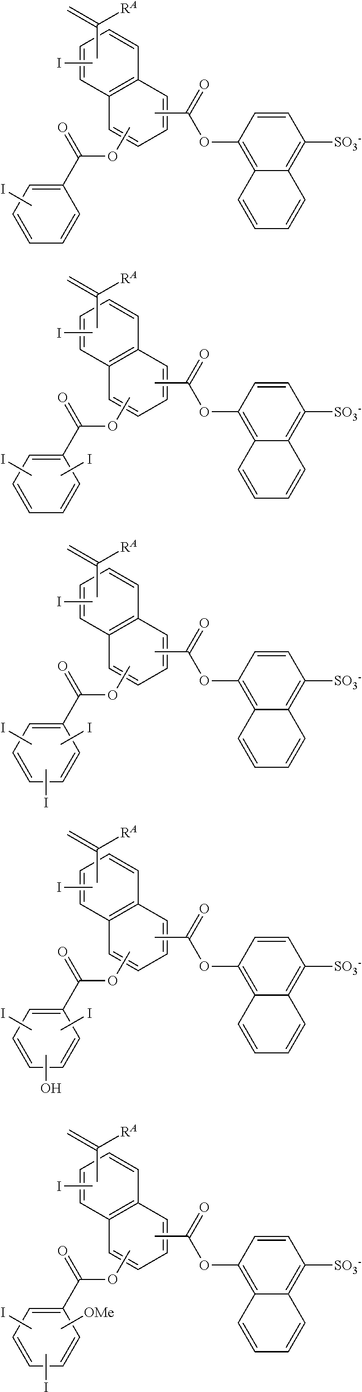

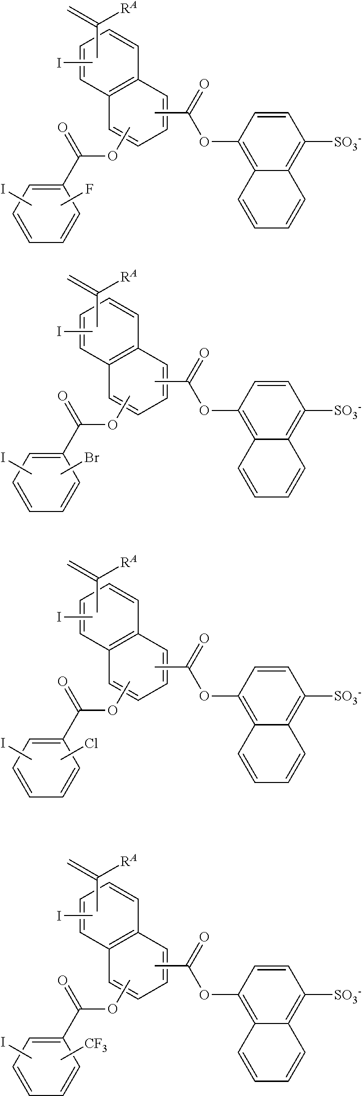

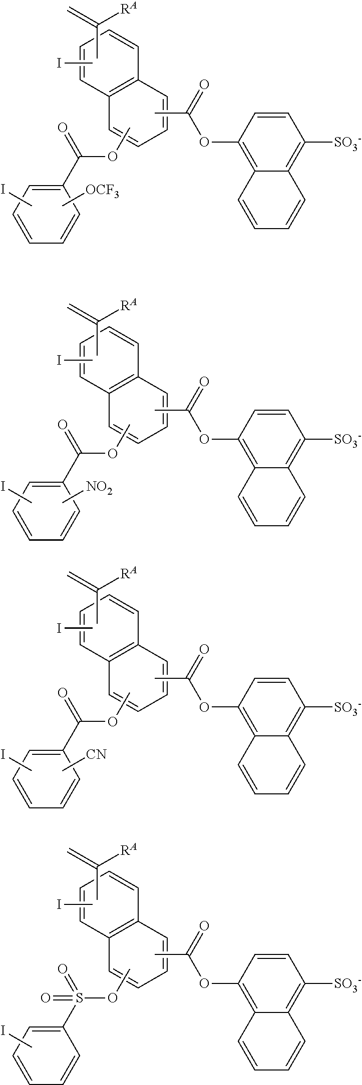

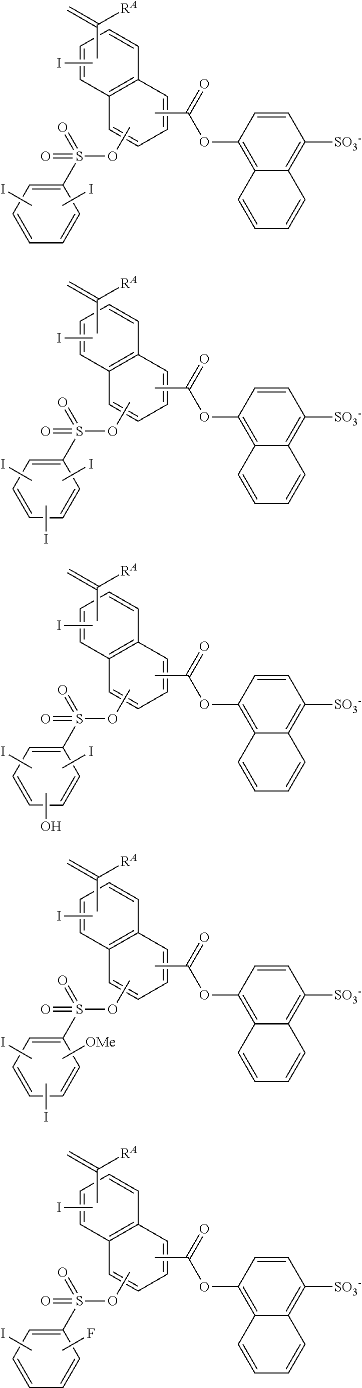

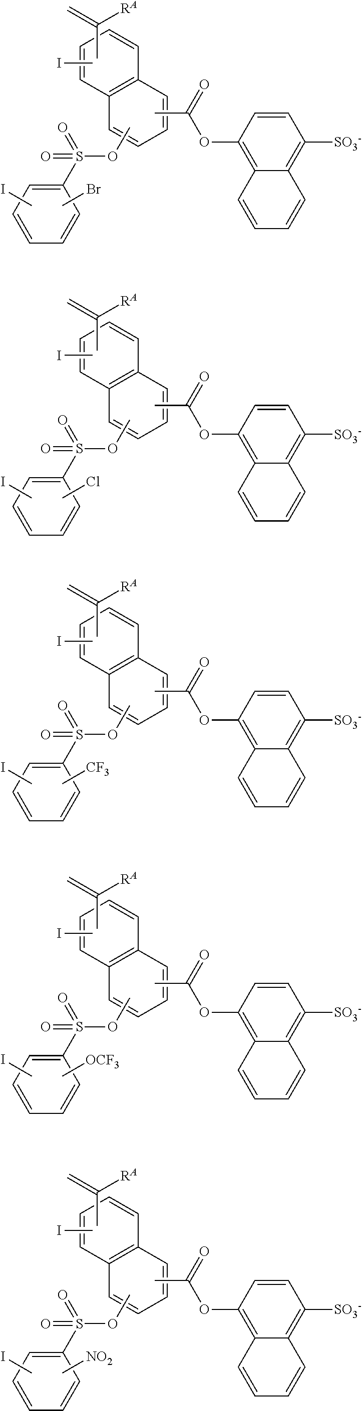

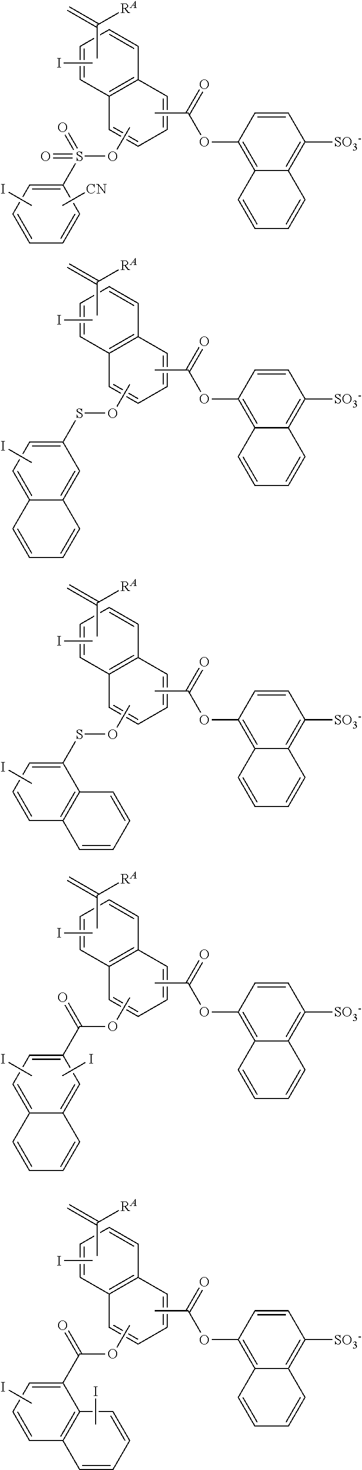

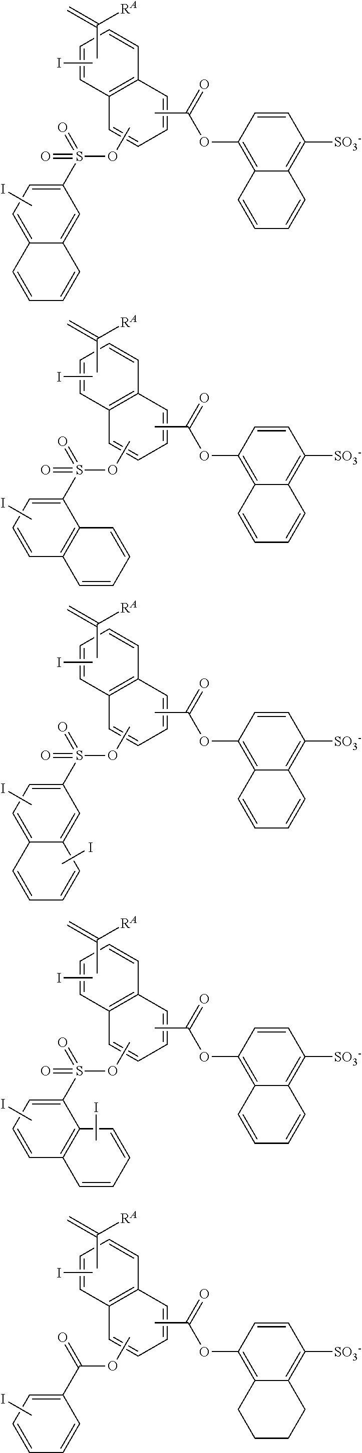

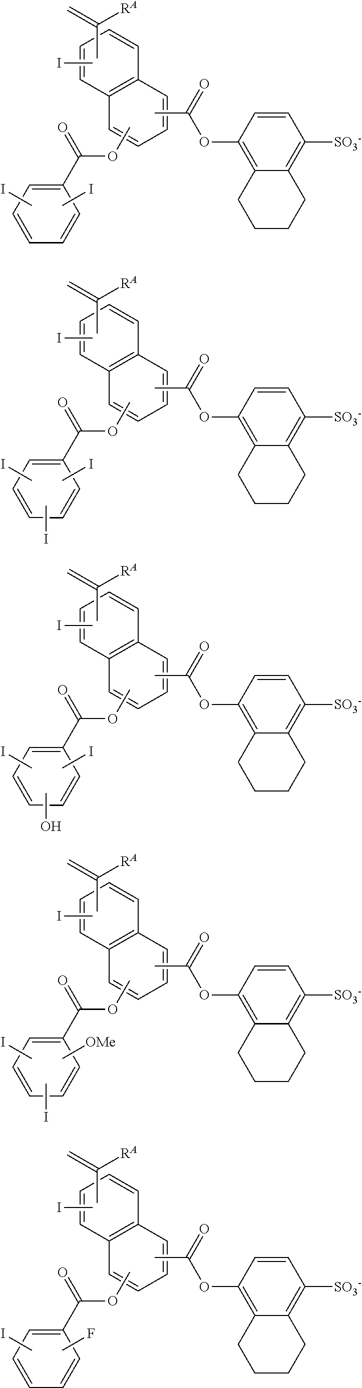

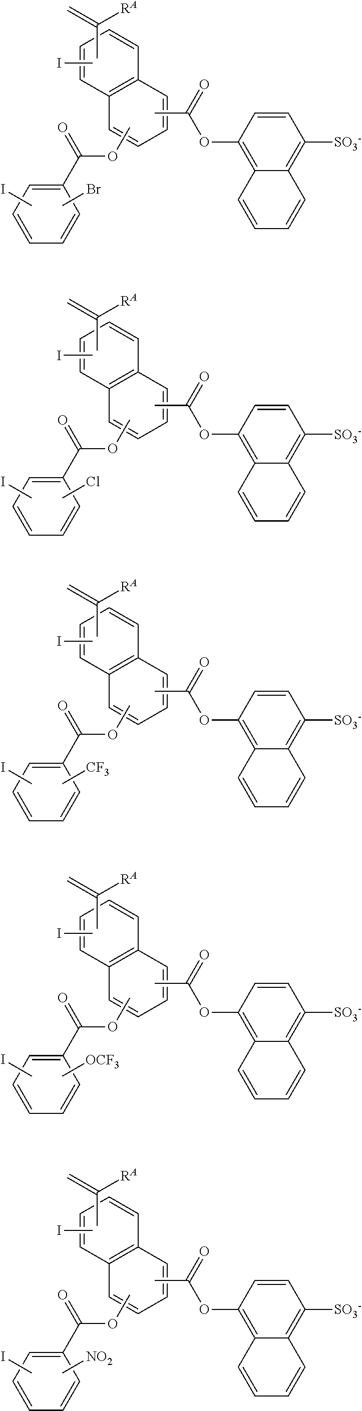

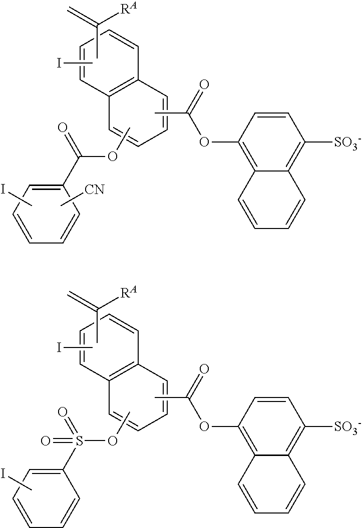

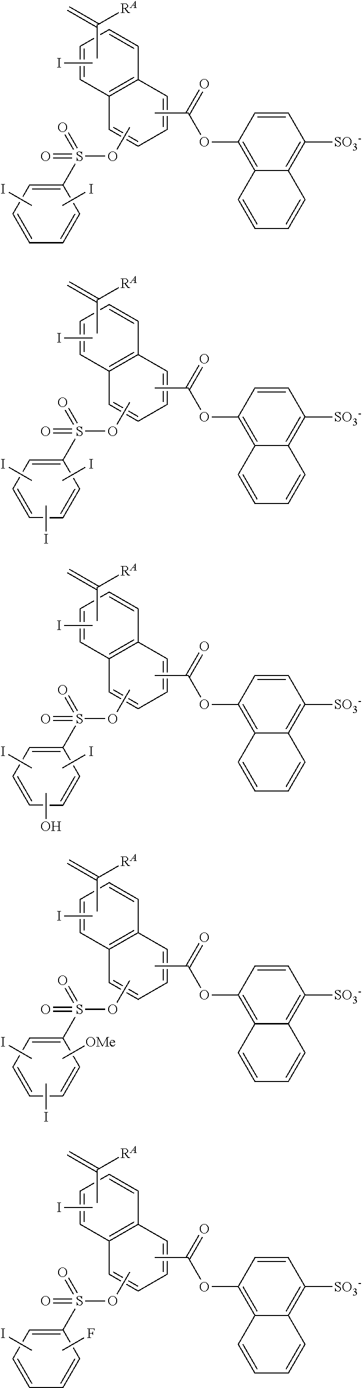

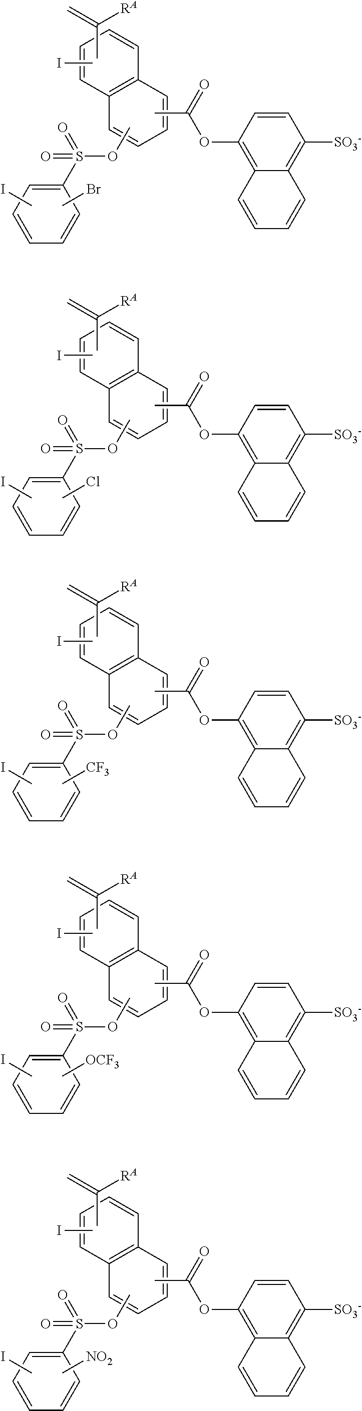

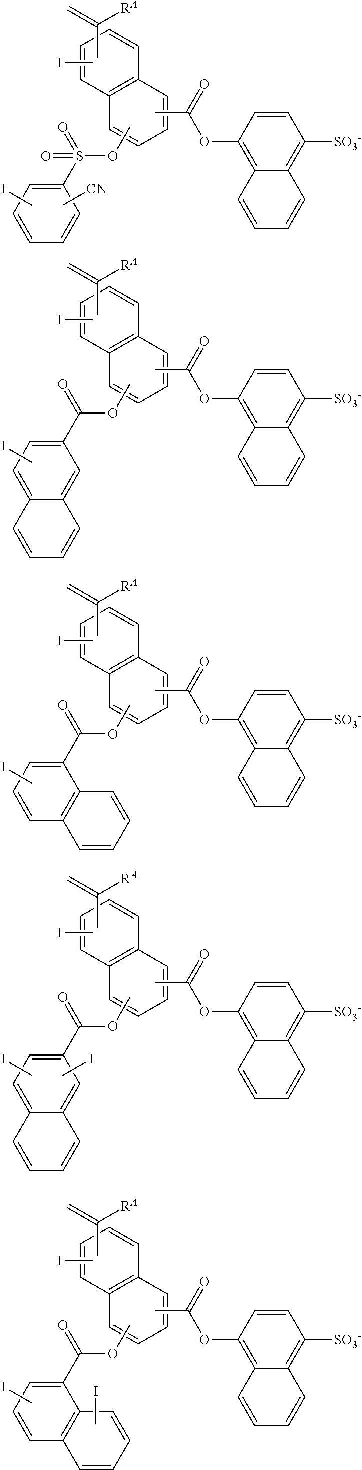

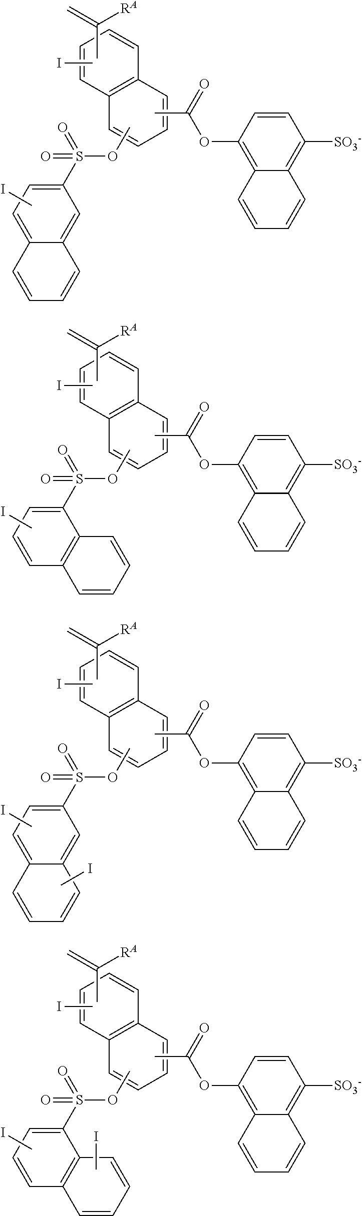

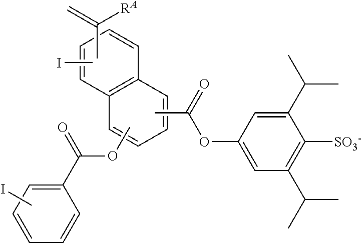

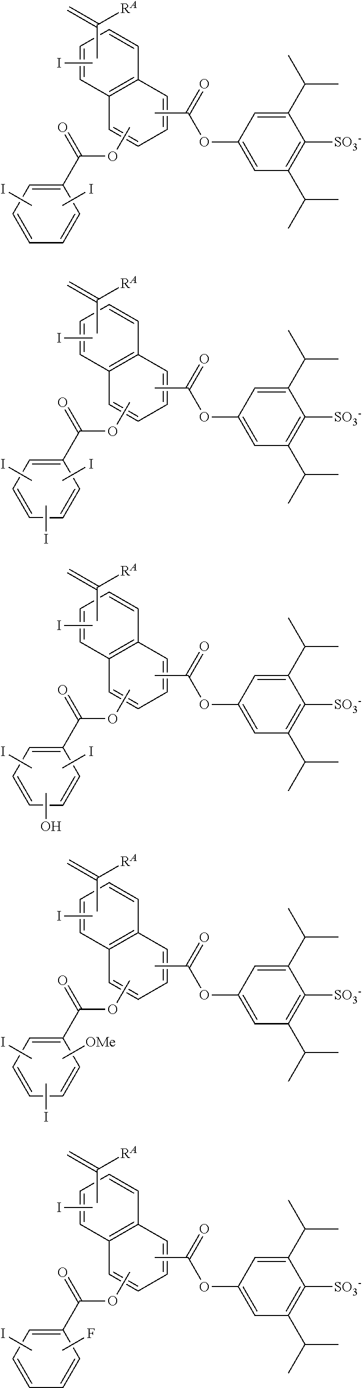

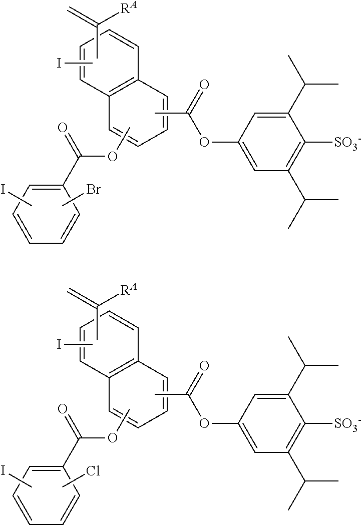

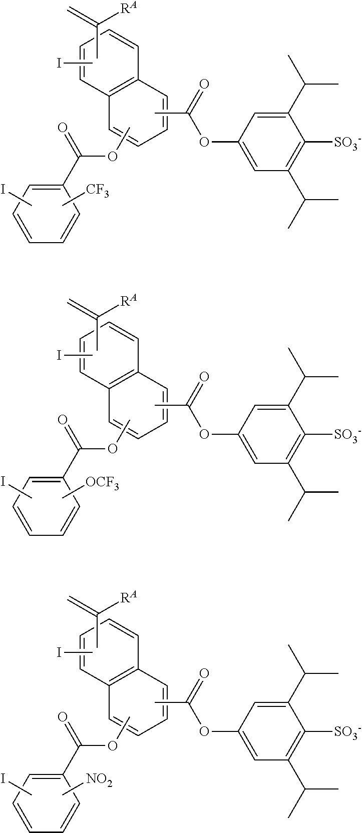

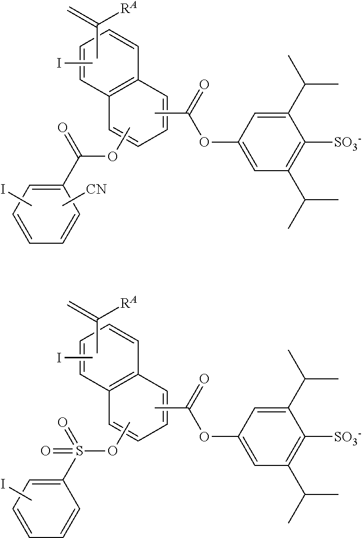

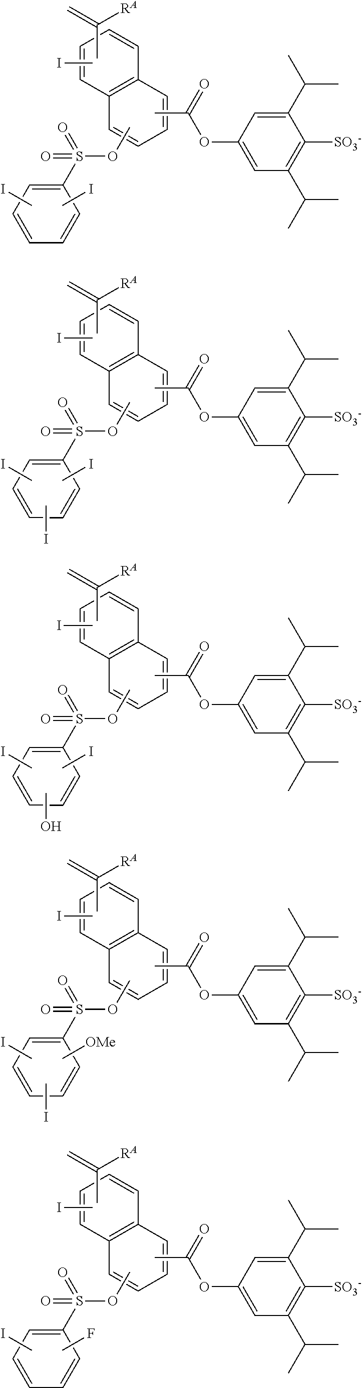

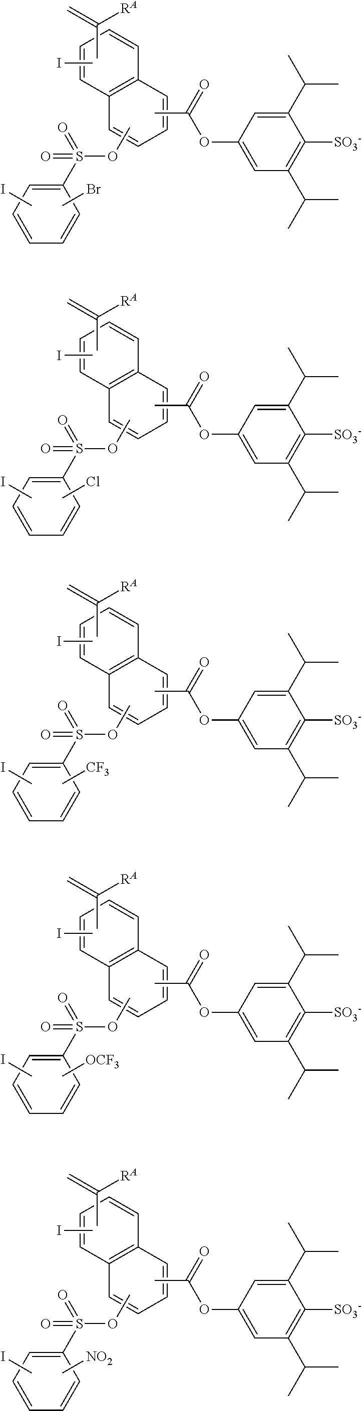

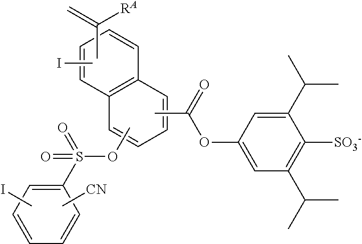

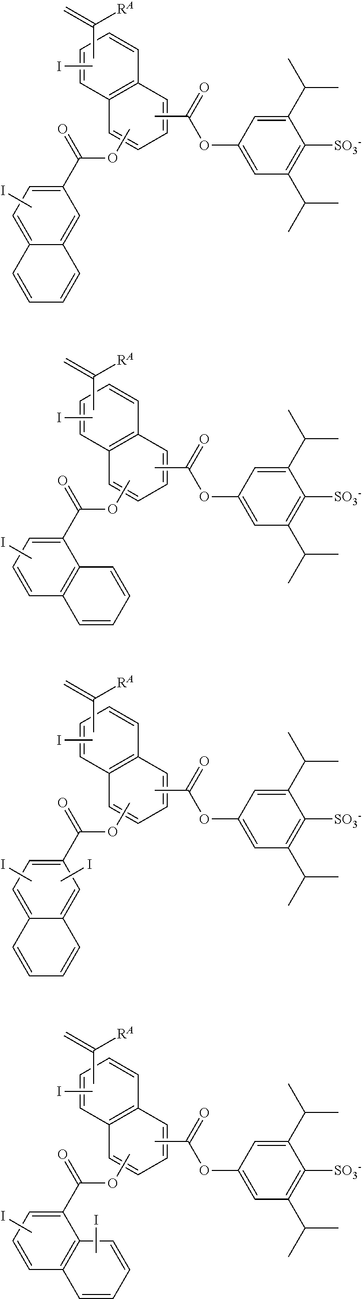

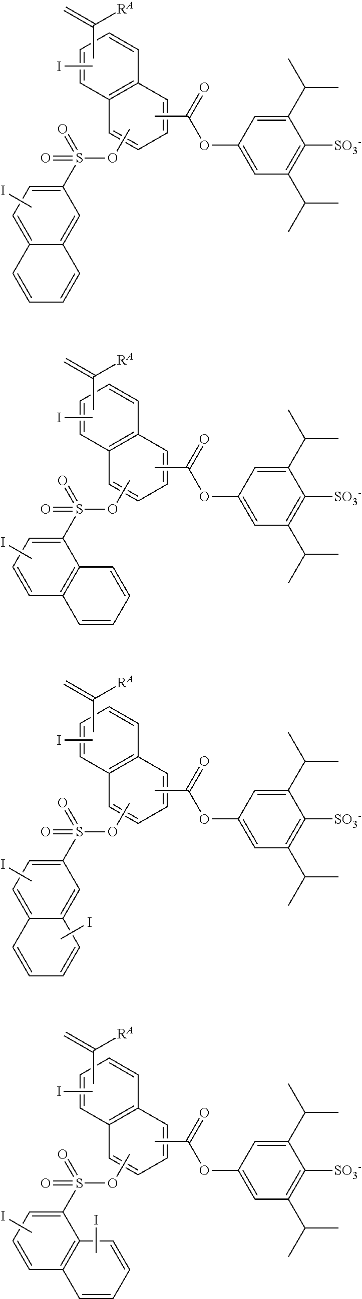

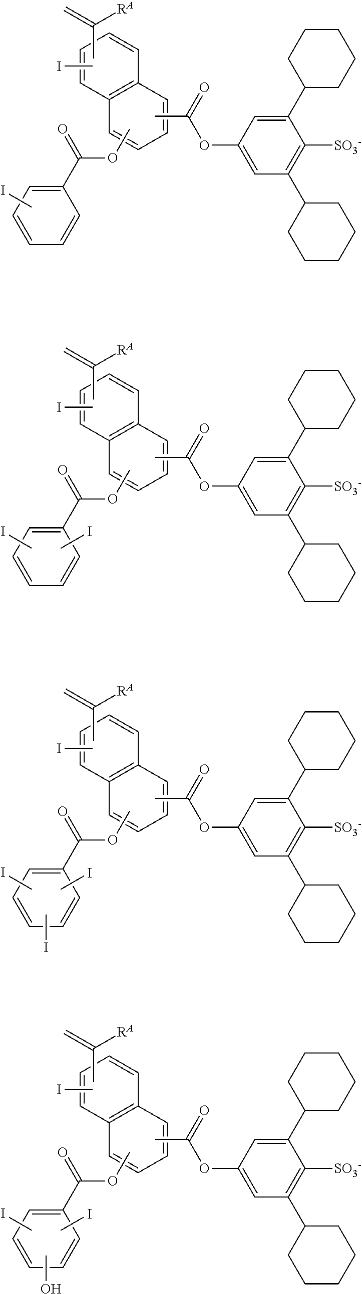

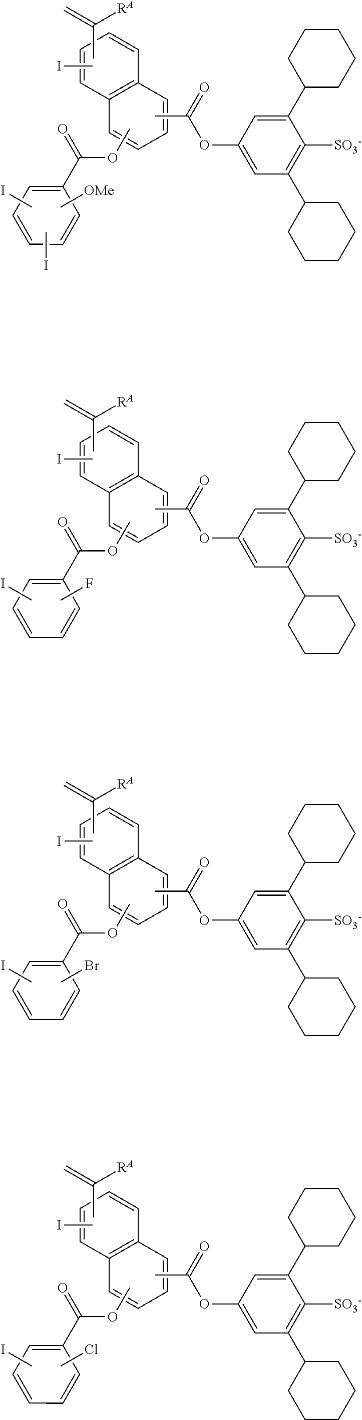

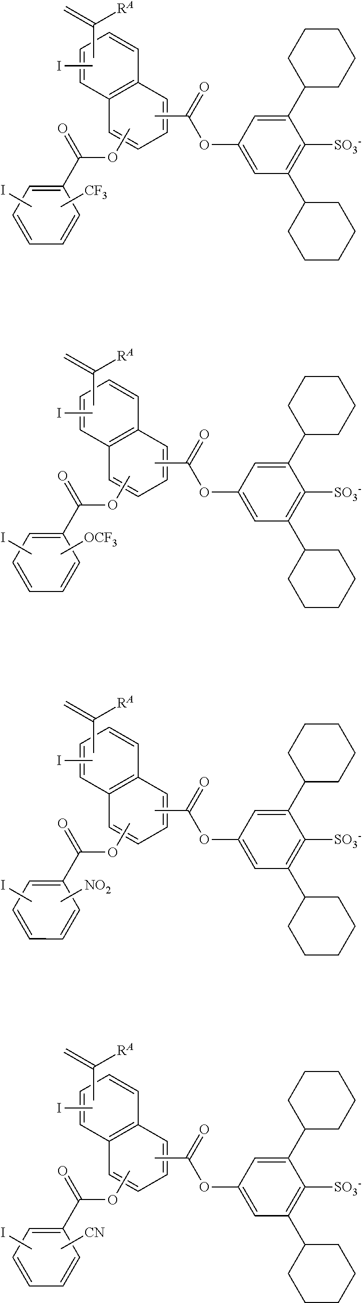

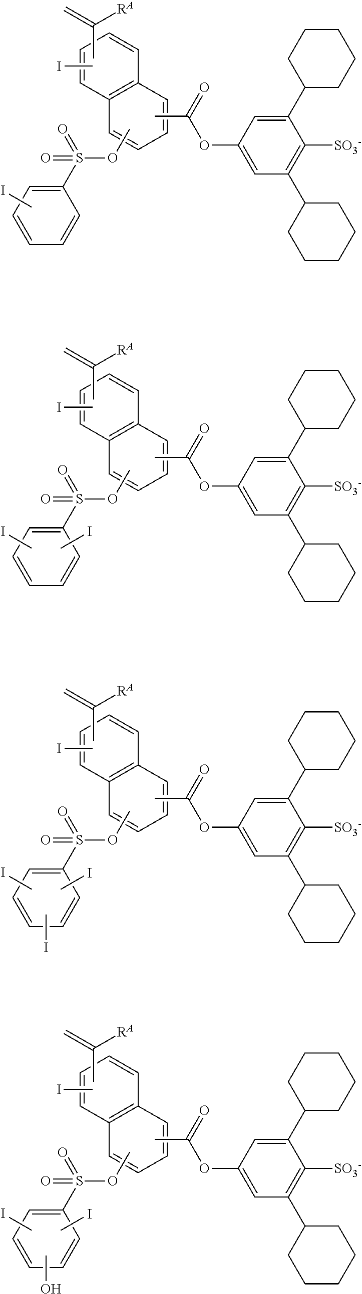

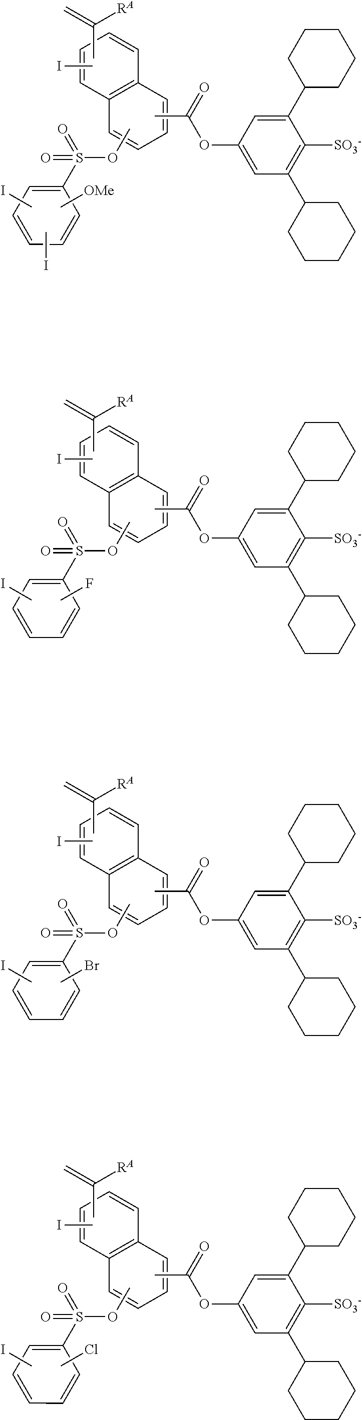

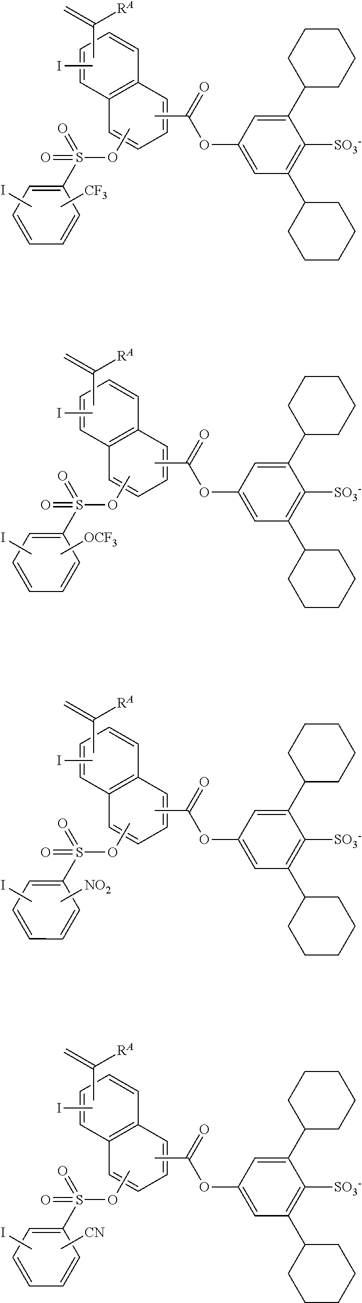

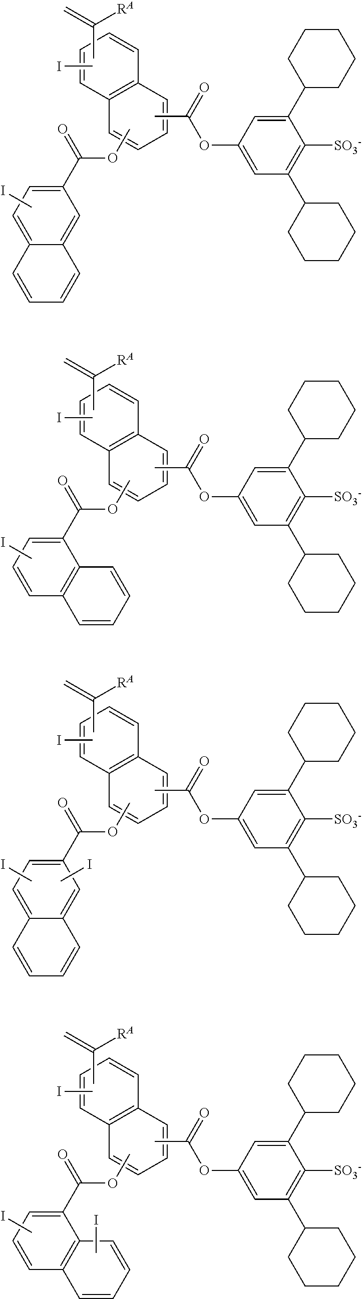

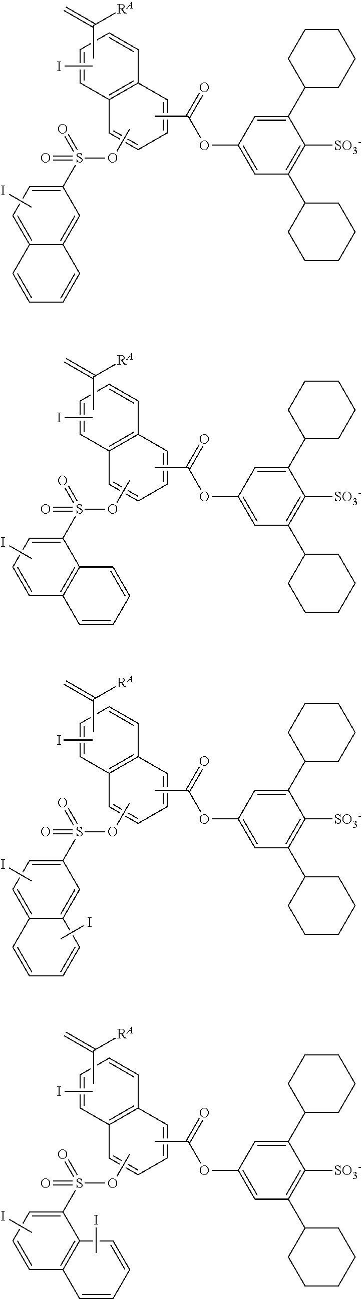

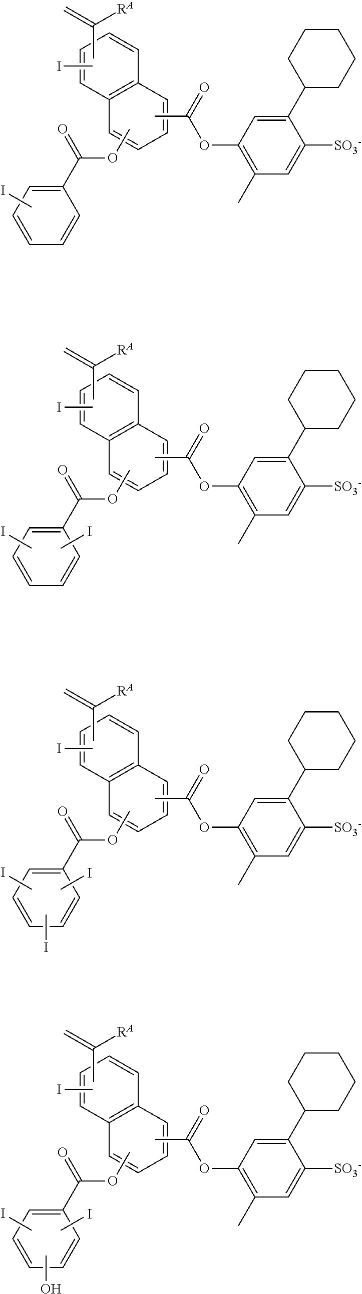

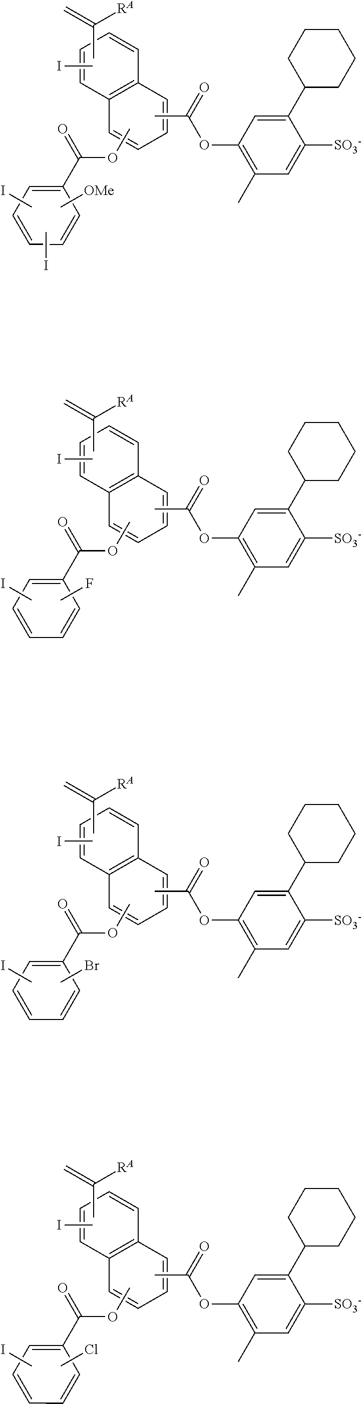

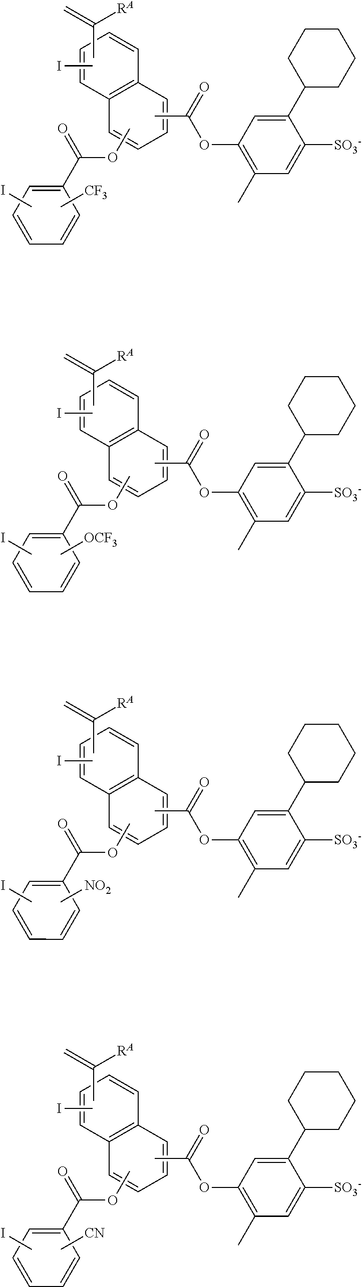

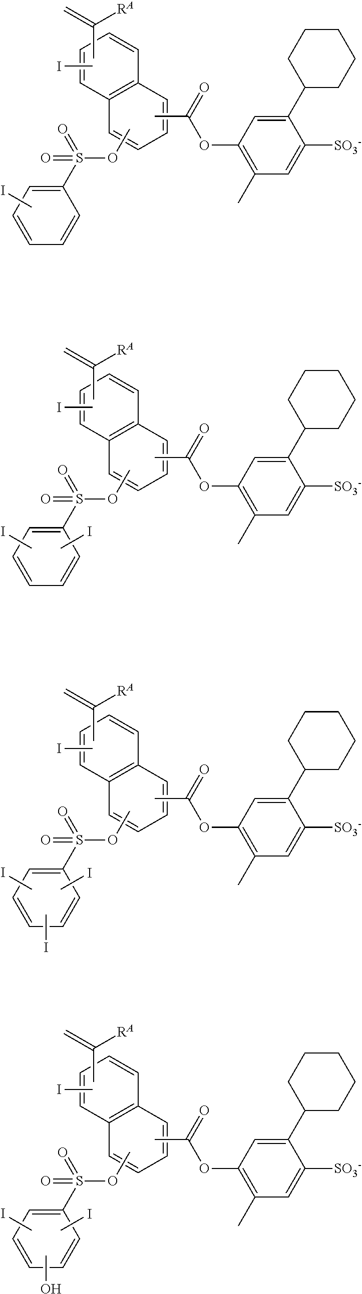

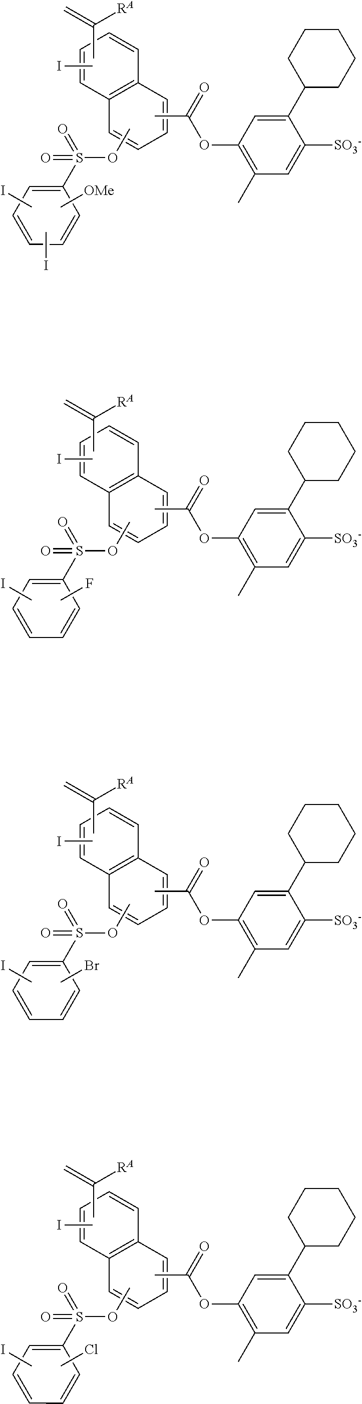

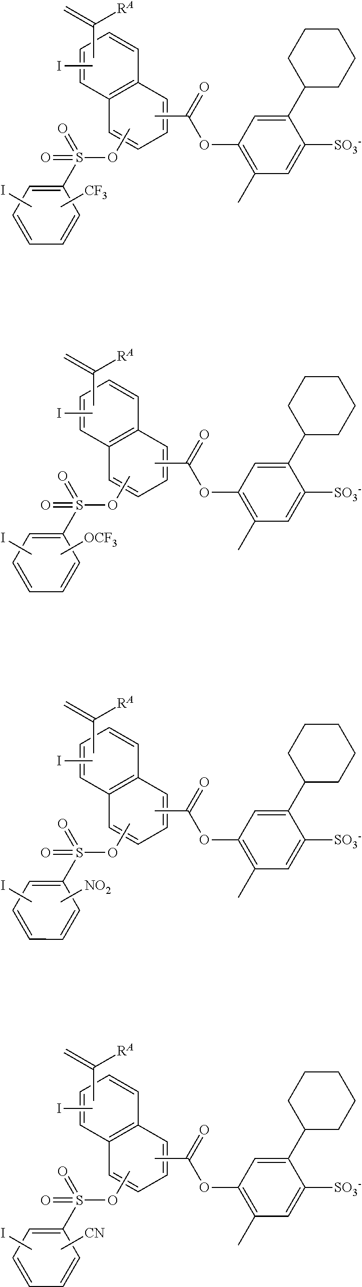

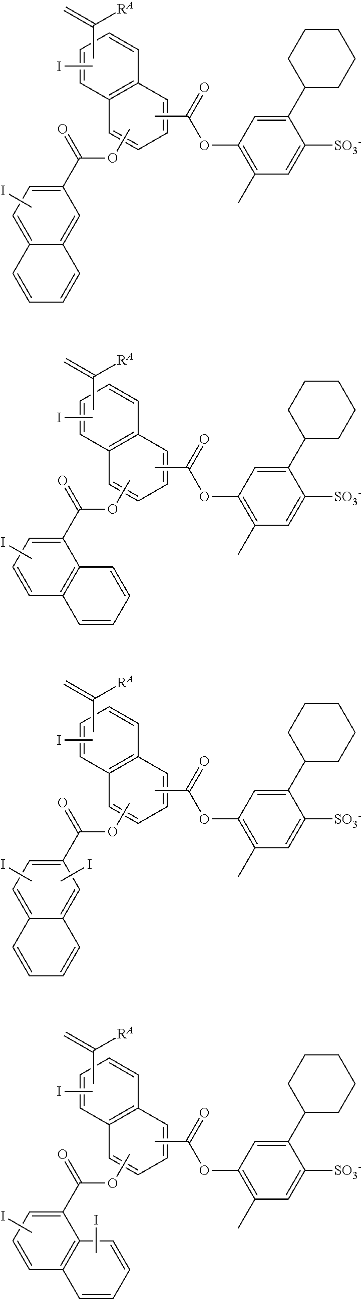

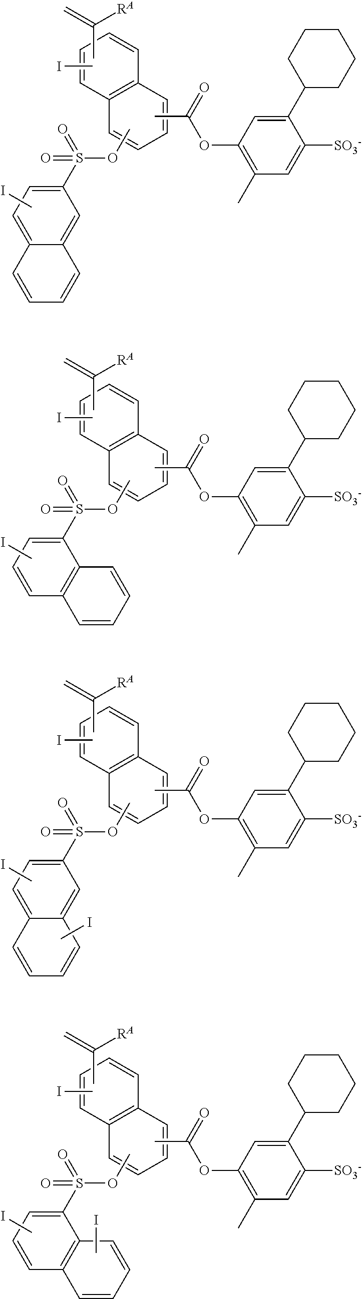

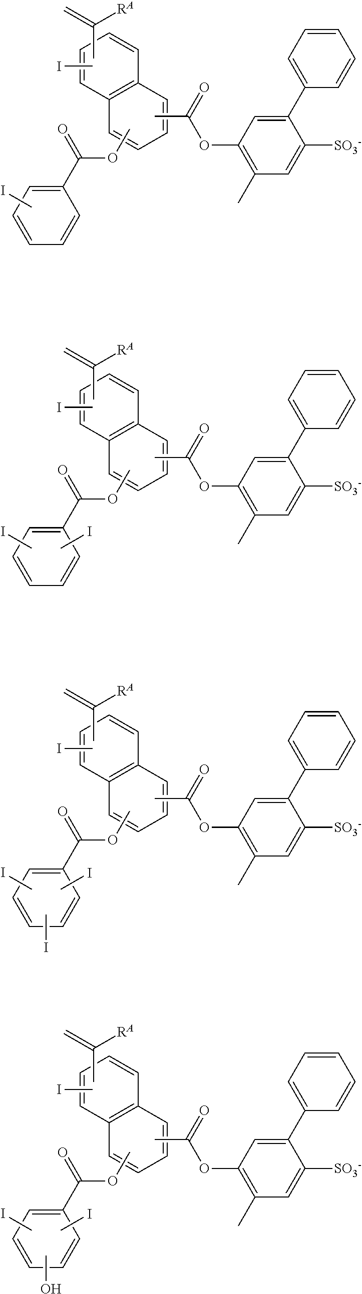

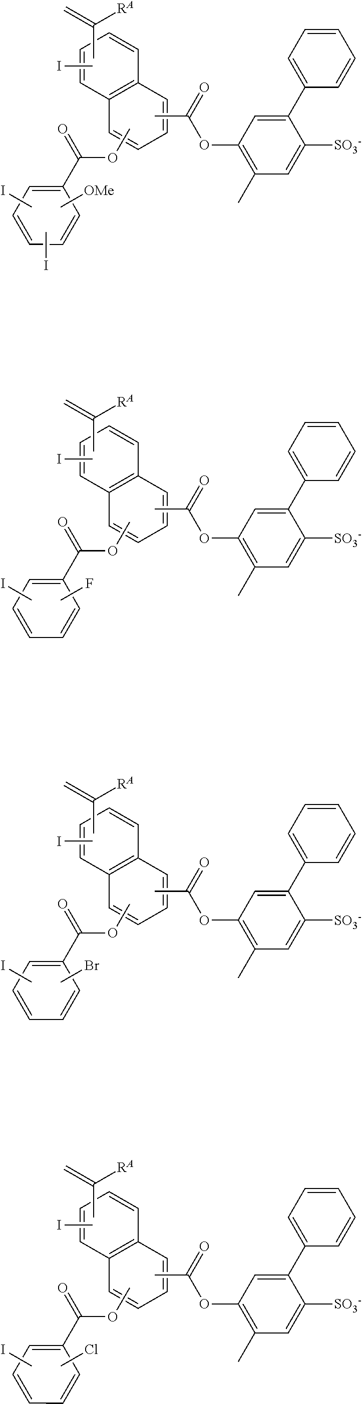

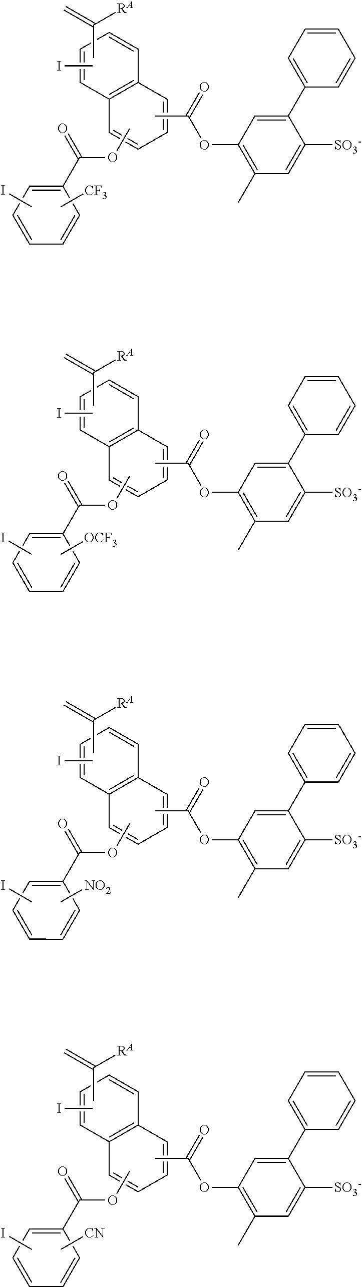

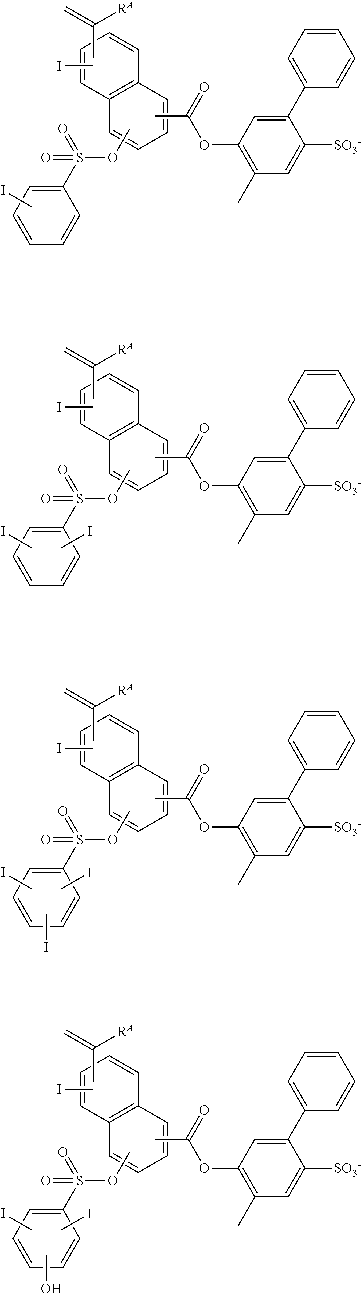

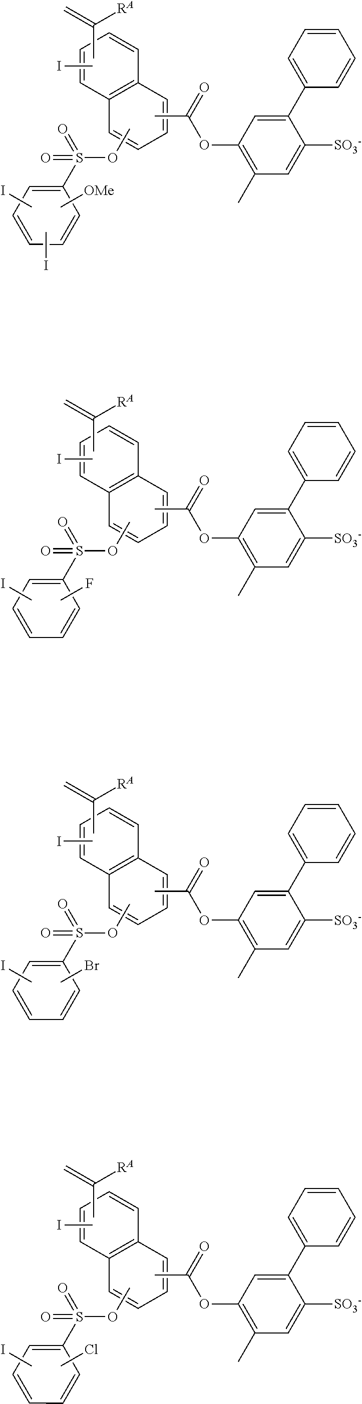

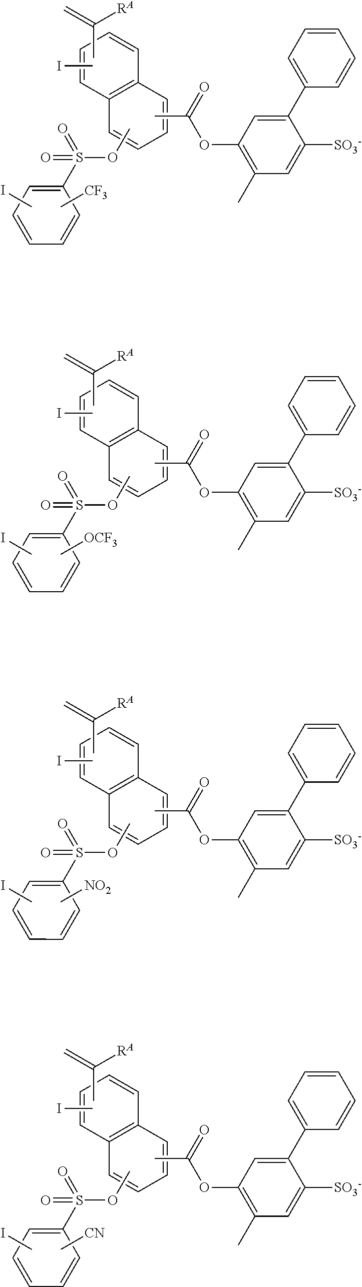

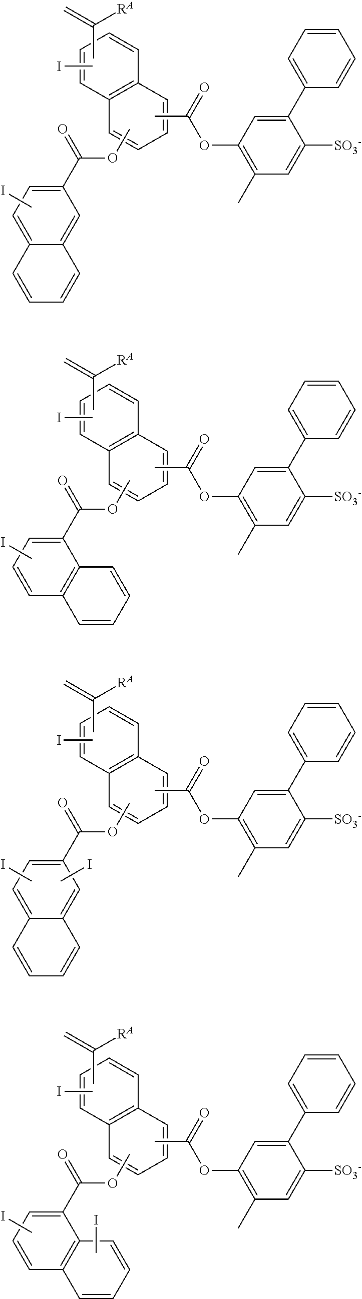

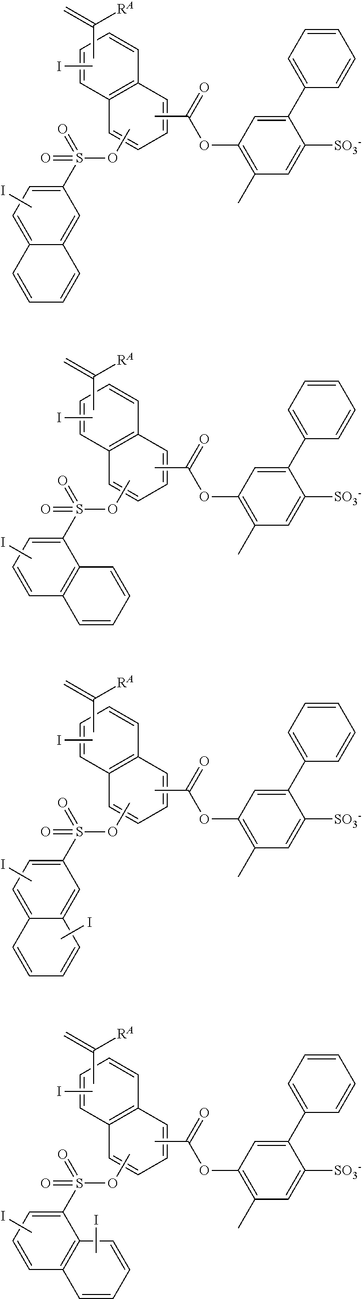

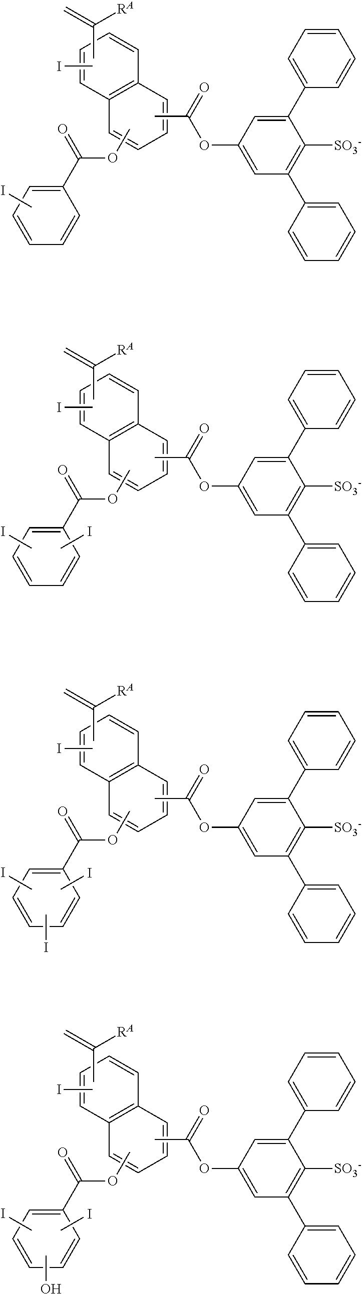

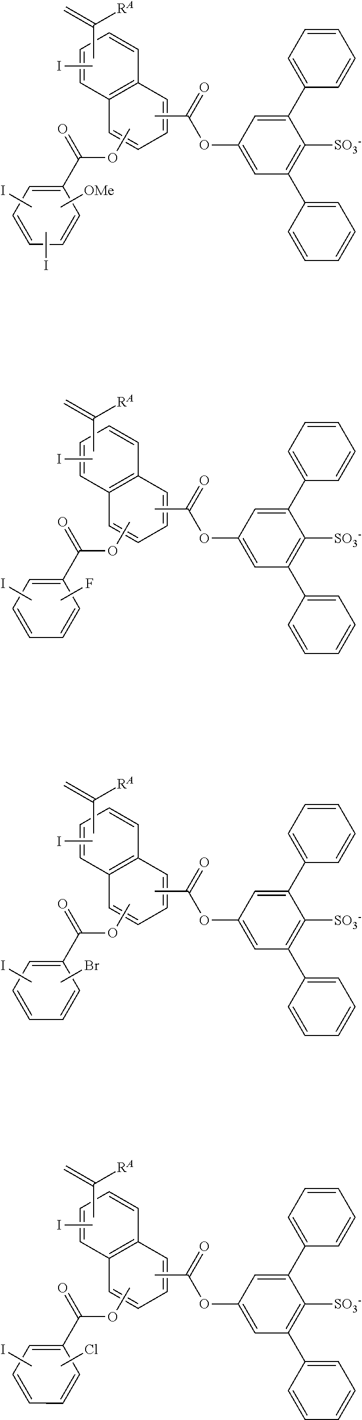

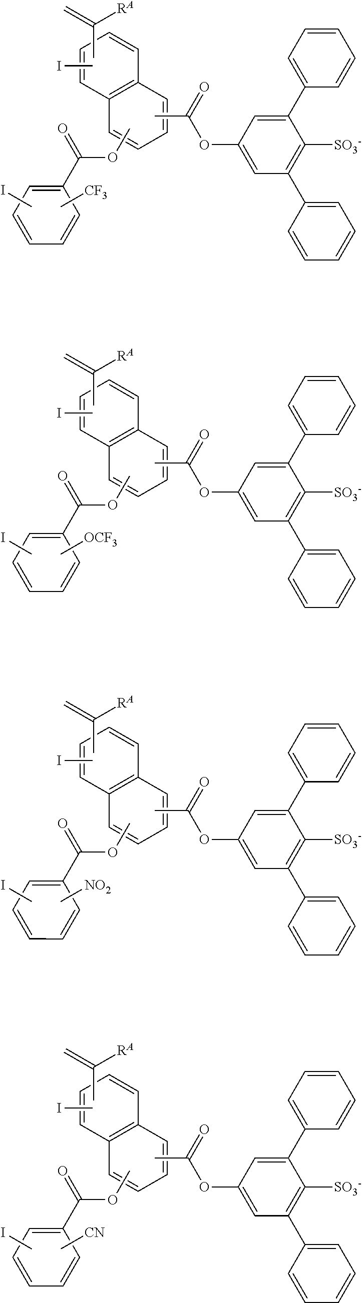

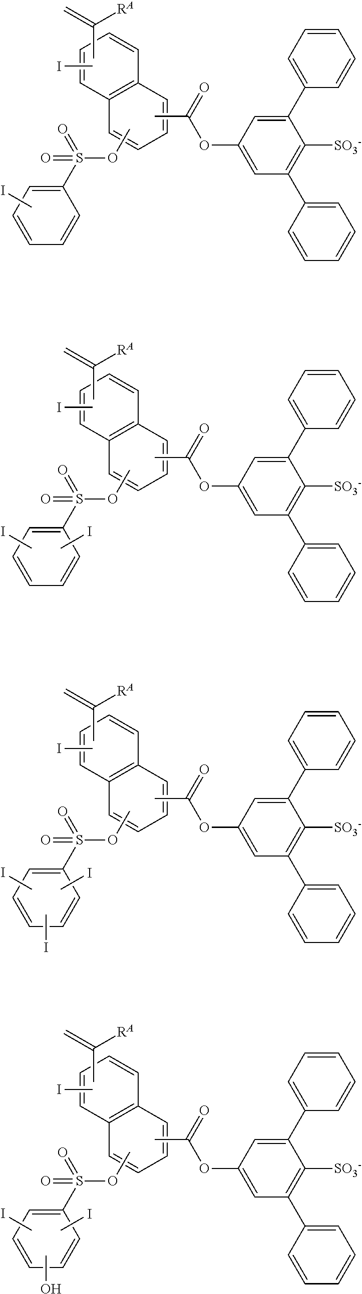

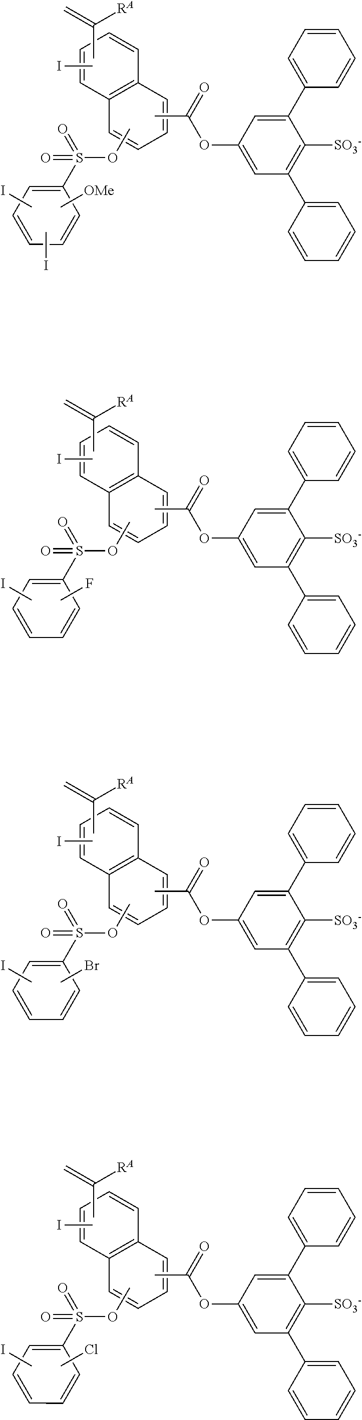

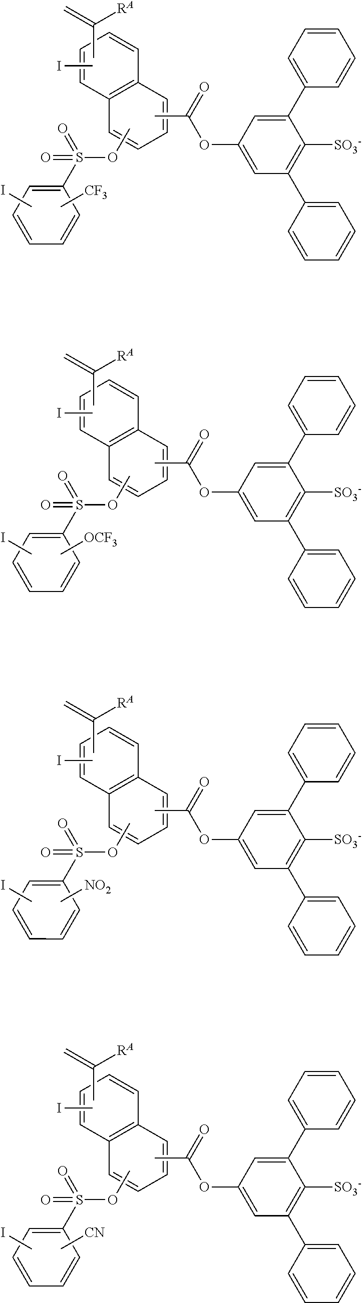

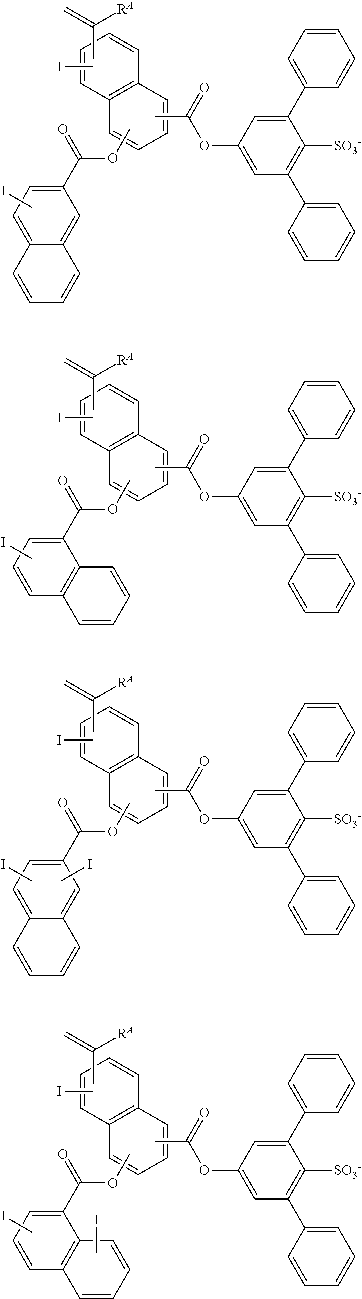

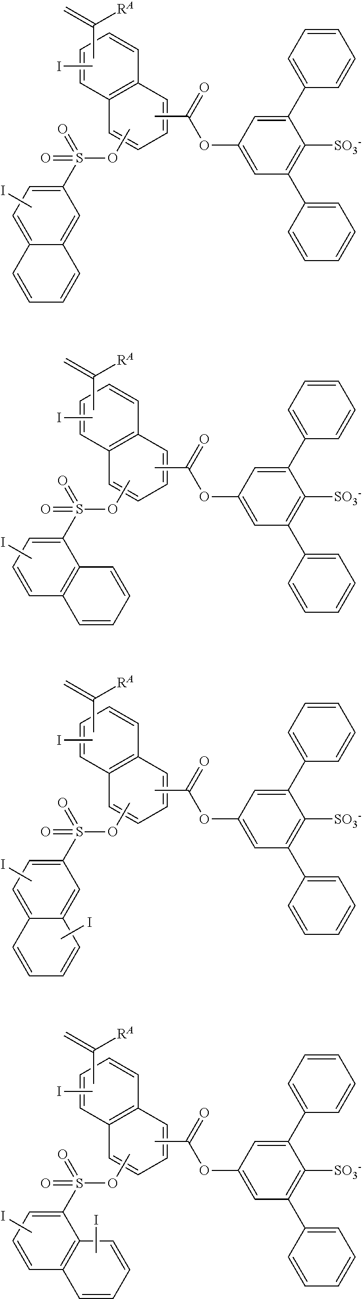

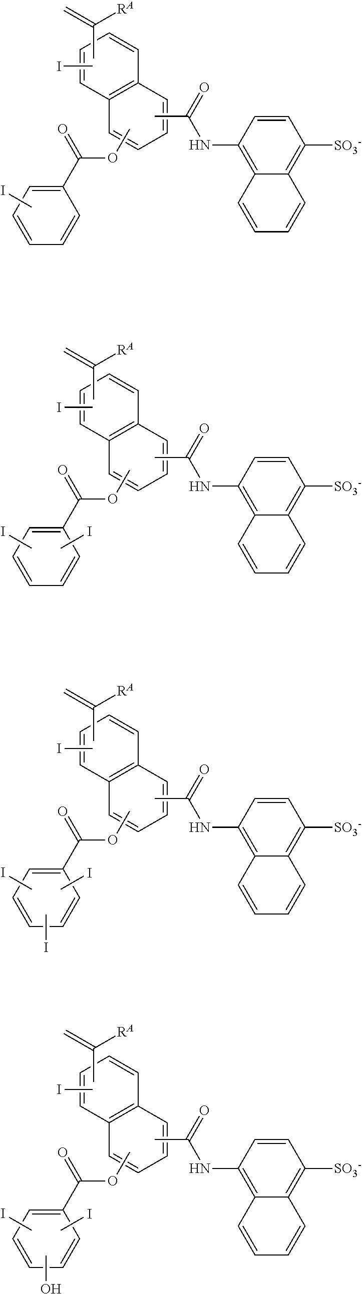

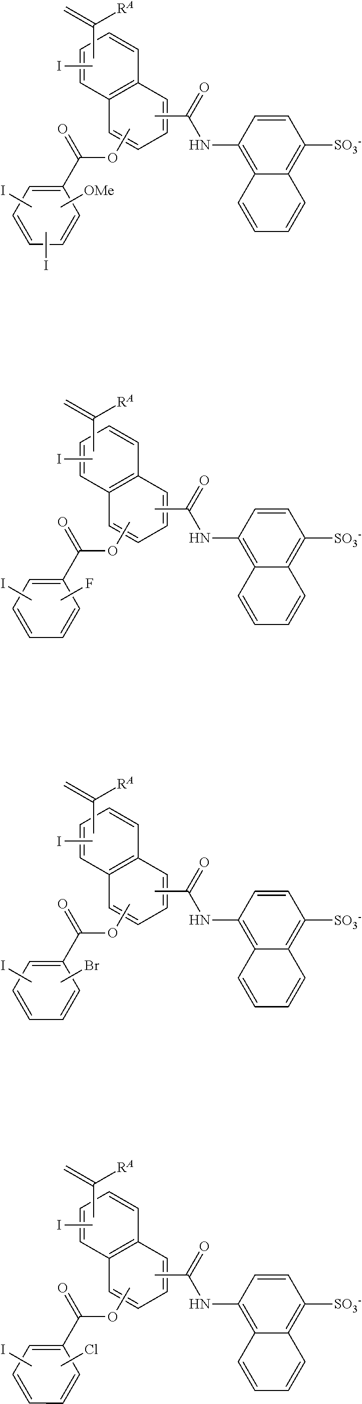

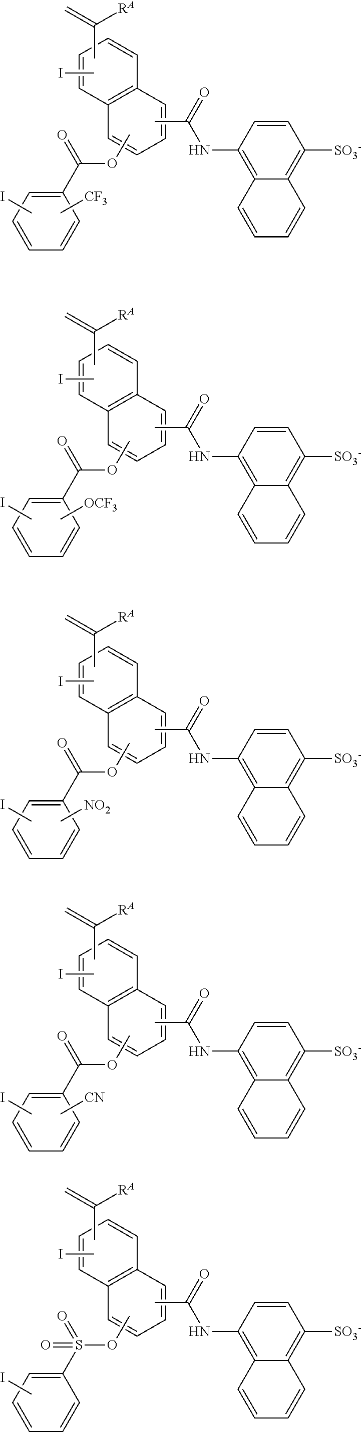

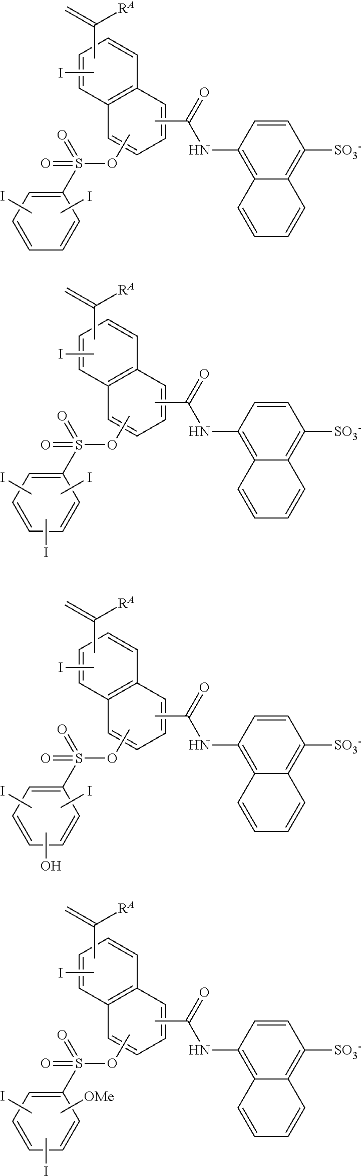

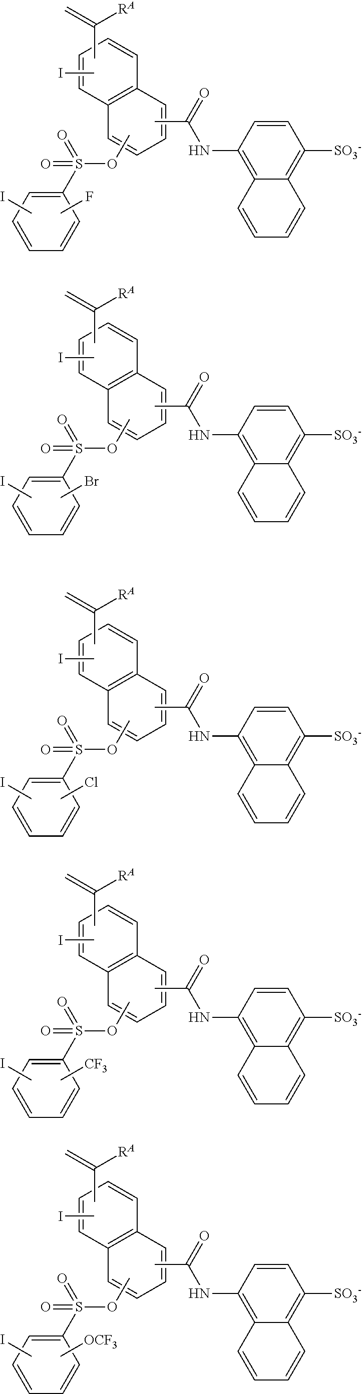

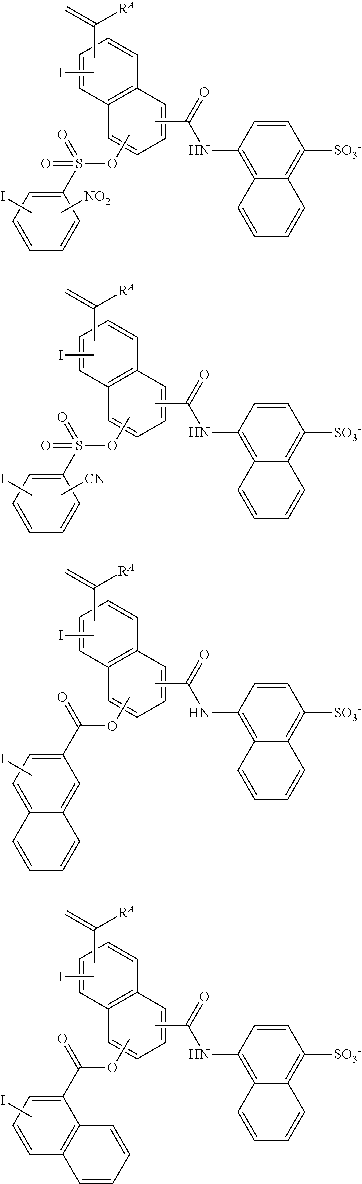

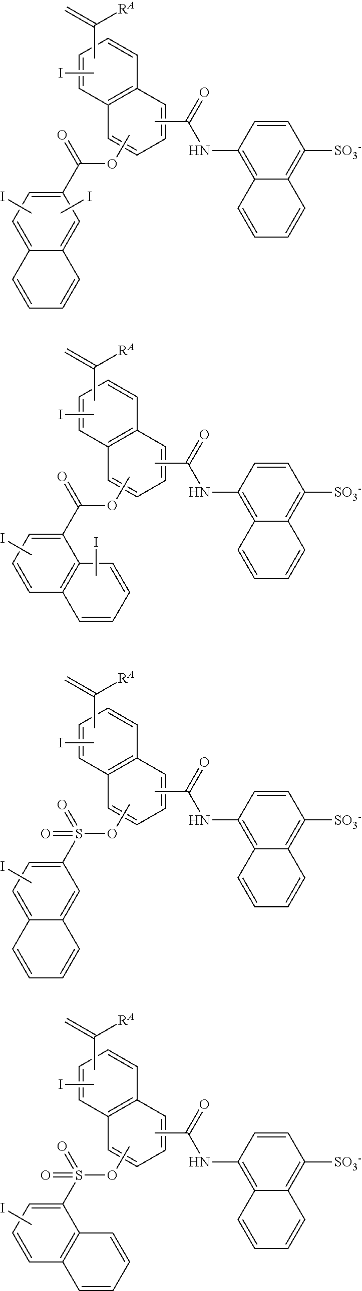

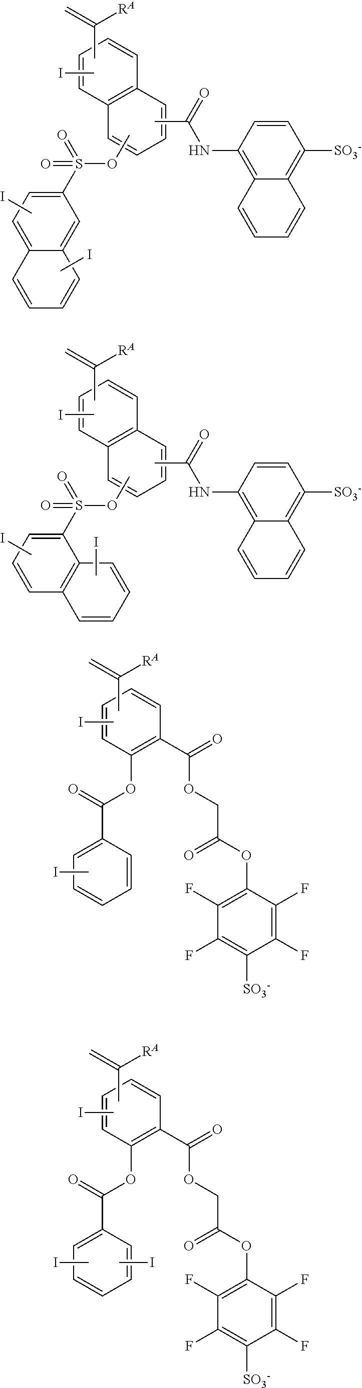

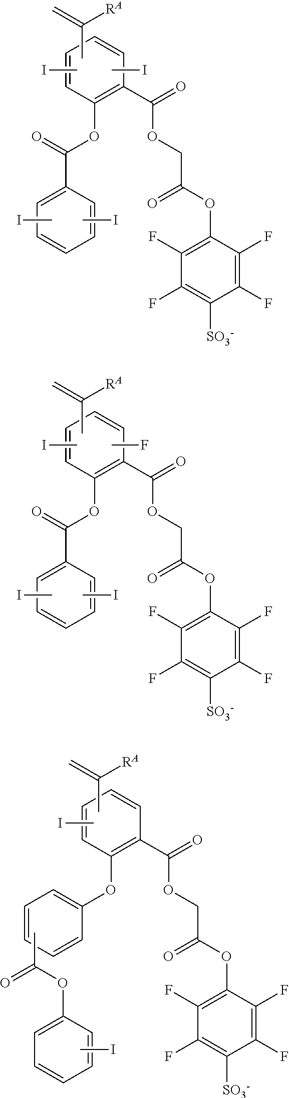

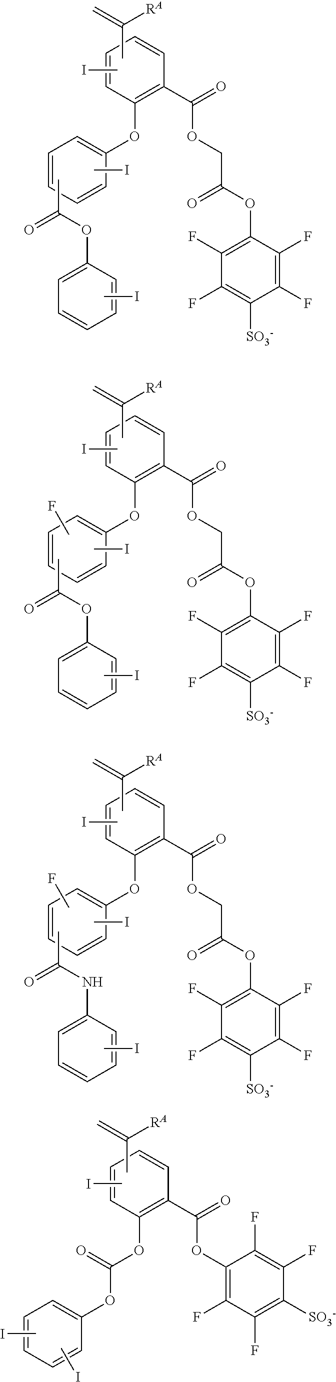

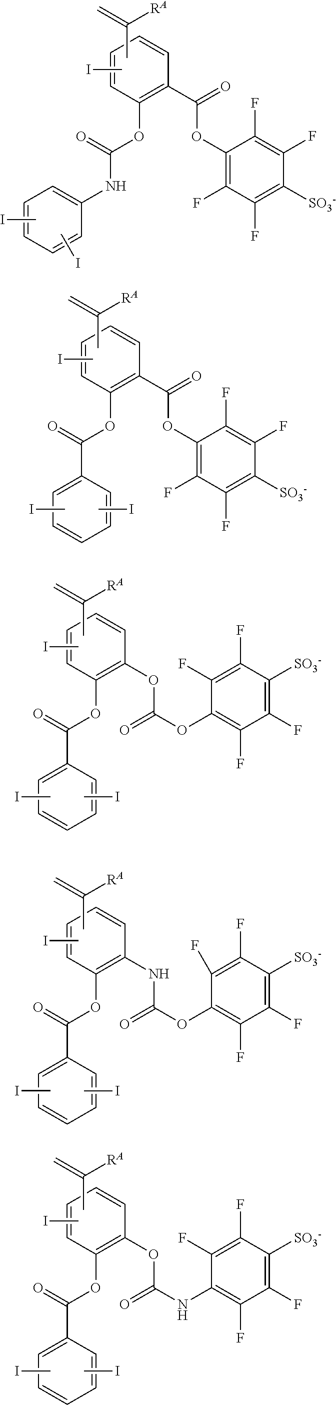

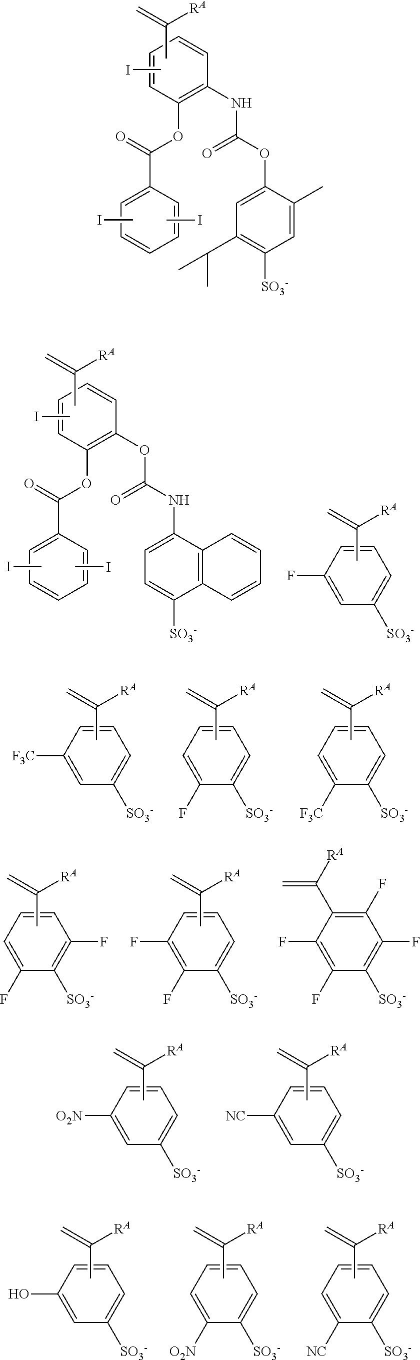

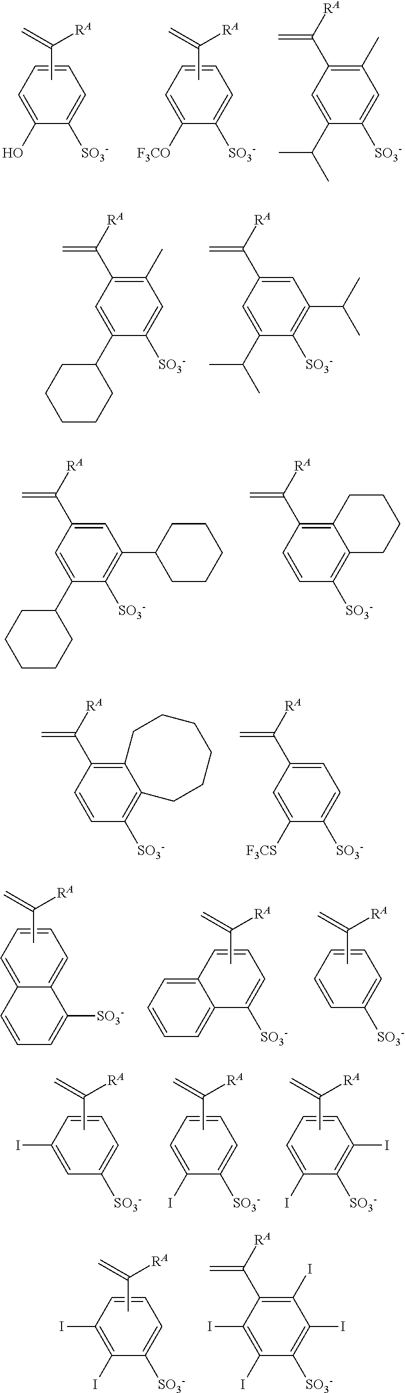

Examples of the specific structure of the sulfonium salt type monomer of the present invention include arbitrary combinations of anions with cations, both as exemplified above.

The sulfonium salt type monomer of the present invention can be synthesized by a known method. For example, first, a sulfonium salt containing the sulfonium cation is synthesized by the synthesis method described in ARKIVOC (Gainesville, FL, United States) (2022), (7), 7-18. Next, by subjecting the synthesized sulfonium salt and the corresponding anion to a salt exchange reaction, the sulfonium salt can be converted into an intended sulfonium salt. The salt exchange with the corresponding anion can be readily performed by a known method, for example, with reference to JP-A 2007-145797.10

The producing method is merely exemplary and the method for producing a sulfonium salt of the present invention is not limited thereto.

Examples of the structural characteristics of the sulfonium salt type monomer of the present invention include that a cyano group is bonded on an aromatic ring of a sulfonium cation together with an aromatic vinyl structure as a polymerizable group and an aromatic sulfonic acid anion structure having an iodine atom. The aromatic sulfonic acid anion structure is more rigid than the alkanesulfonic acid anion structure, and has a feature of less acid diffusion. For iodine atoms, particularly in EUV lithography having a wavelength 13.5 nm, secondary electrons are generated from the iodine atoms during exposure because the absorption of EUV by the iodine atoms is very large. Since the sulfonium salt type monomer of the present invention has a polymerizable group in an anion part, the polymer of the present invention obtained using the sulfonium salt type monomer becomes an anion-bound acid generator in which an anion side is bonded to a polymer main chain. That is, since an acid bonded to the main chain of the polymer is generated, diffusion of the generated acid can be suppressed. In particular, a polymerizable group having a styrene or vinylnaphthalene structure is more rigid than a polymerizable group such as a methacrylic acid ester and improves the glass transition temperature (Tg) of the polymer. It is considered that the aromatic rings in the polymer or between the polymers interact with each other (exhibits a x-x stacking effect) to regularly arrange the polymers, and even in fine pattern formation, resistance to pattern collapse is exhibited against the developer. In an etching step after fine pattern formation, excellent etch resistance is also exhibited because the aromatic ring is directly bound to the backbone. On the other hand, a cyano group substituted on an aromatic ring of a sulfonium cation is known as a strong electron-withdrawing group. From this, it is considered that the energy level of the LUMO in the frontier orbital theory decreases. Therefore, the secondary electrons generated from the iodine atoms in the anion are easily received, the decomposition of the cation is promoted, and the acid is efficiently generated. Since it has a lone pair of electrons on the nitrogen atom of the cyano group, it can be expected that it interacts with the proton of the generated acid to function as an acid diffusion inhibiting group. These effects synergistically can work to increase sensitivity, prevent deterioration of resolution due to blurring of acid diffusion, and improve LWR and CDU. Thus, the polymer of the present invention is particularly suitable as a material for a chemically amplified positive resist composition.

### Polymer

The polymer of the present invention comprises repeat units derived from a sulfonium salt type monomer having formula (A)(also referred to as repeat units A, hereinafter).

The polymer may comprise repeat units having the following formula (a1)(also referred to as repeat units a1, hereinafter) or repeat units having the following formula (a2) (also referred to as repeat units a2, hereinafter).

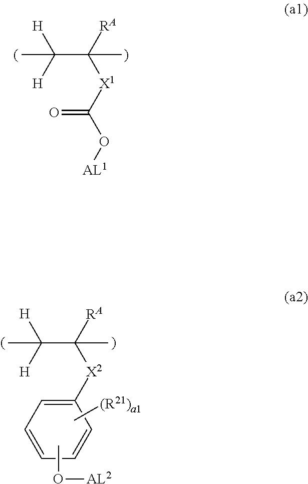

In formulae (a1) and (a2), R^{A} is each independently a hydrogen atom, a fluorine atom, a methyl group, or a trifluoromethyl group.

In formula (a1), X¹ is a single bond, a phenylene group, a naphthylene group, or *—C(═O)—O—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom. X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring. * designates a point of attachment to the carbon atom in the backbone.

In formula (a2), X² is a single bond or *—C(═O)—O—. * designates a point of attachment to the carbon atom in the backbone. R²¹ is a halogen atom, a cyano group, a hydroxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. a1 is 0, 1, 2, 3, or 4 and preferably 0 or 1. When a1 is 2, 3, or 4, respective R²¹ may be the same as or different from each other.

In formulae (a1) and (a2), AL¹ and AL² are each independently an acid labile group. Specific examples of the acid labile group include those groups described in JP-A 2013-80033 and JP-A 2013-83821.

Typically, specific examples of the acid labile group include groups having the following formulae (AL-1) to (AL-3).

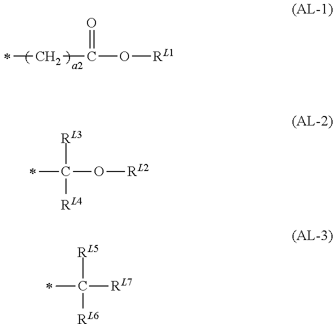

wherein * denotes a point of attachment.

In formulae (AL-1) and (AL-2), R^{L1} and R^{L2} are each independently a C₁-C₄₀ hydrocarbyl group which may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ saturated hydrocarbyl group.

In formula (AL-1), a2 is an integer of 0 to 10 and preferably 1, 2, 3, 4, or 5.

In formula (AL-2), RL3 and R¹⁴ are each independently a hydrogen atom or a C₁-C₂₀ hydrocarbyl group which may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ saturated hydrocarbyl group. Any two of RL2, RL3, and RIA may bond together to form a C₃-C₂₀ ring with the carbon atom or carbon and oxygen atoms to which they are attached. The ring is preferably a C₄-C₁₆ ring and particularly preferably an alicyclic ring.

In formula (AL-3), RL5, RL6, and RL7 are each independently a C₁-C₂₀ hydrocarbyl group which may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ saturated hydrocarbyl group. Any two of R^{L5}, R^{L6}, and R^{L7} may bond together to form a C₃-C₂₀ ring with the carbon atom to which they are attached. The ring is preferably a C₄-C₁₆ ring and particularly preferably an alicyclic ring.

Specific examples of the repeat units a1 are shown below, but not limited thereto. In the following formula. R^{A} and AL¹ are as defined above.

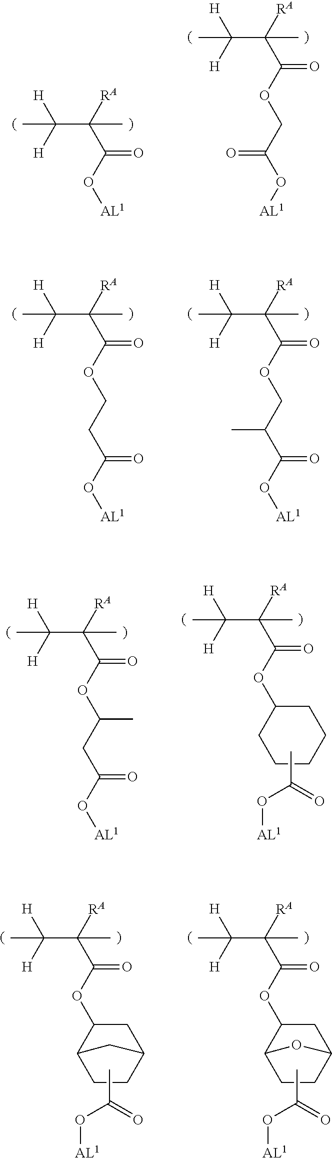

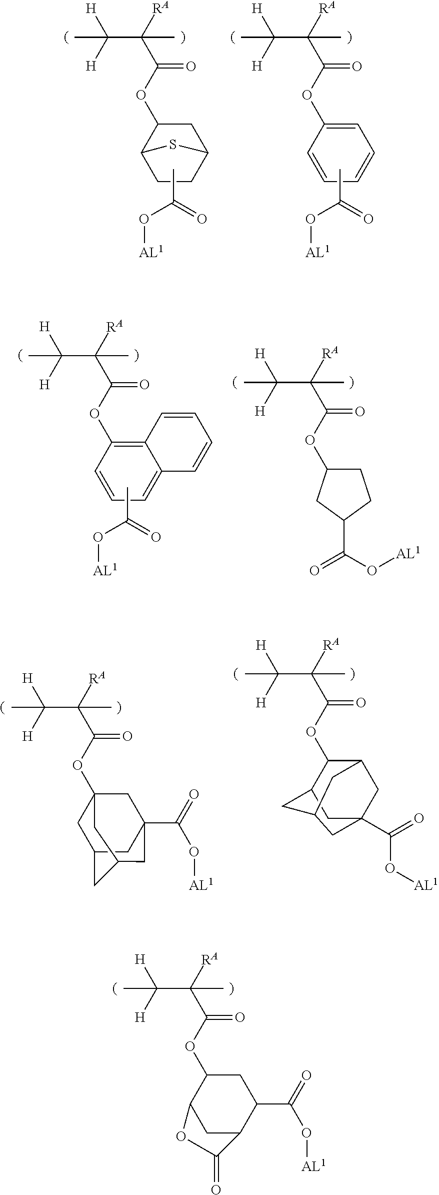

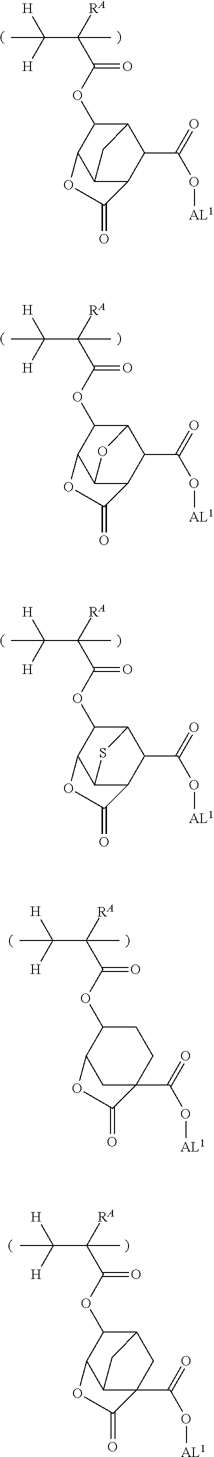

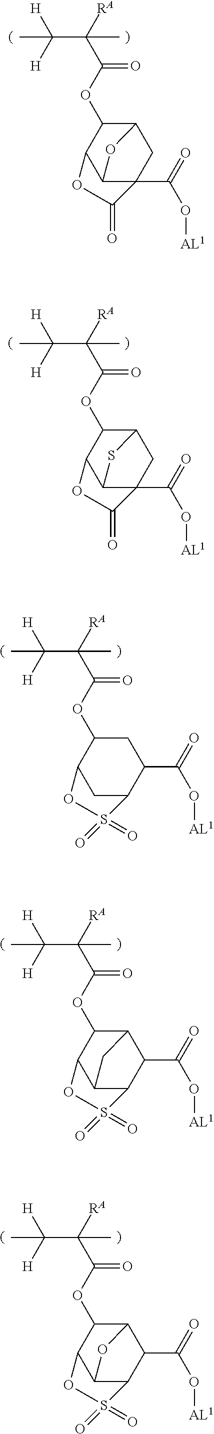

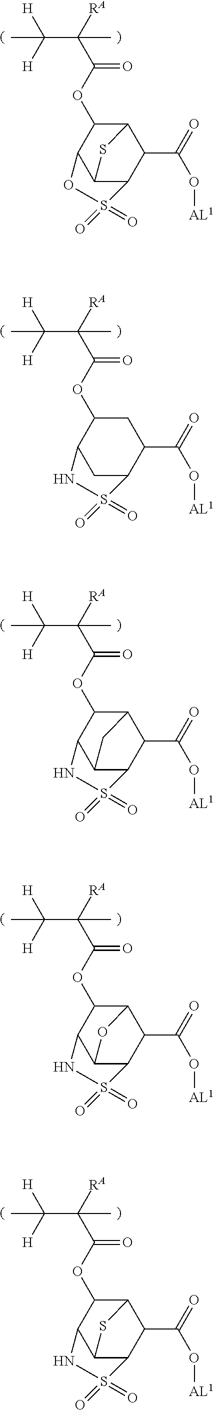

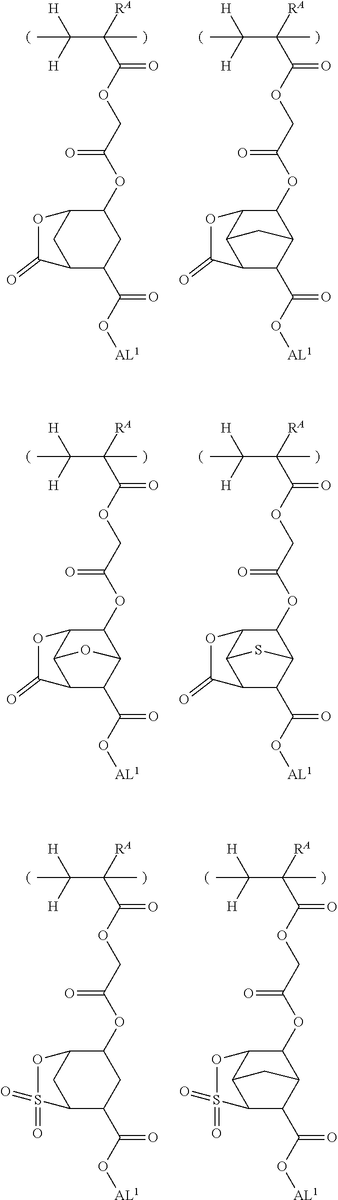

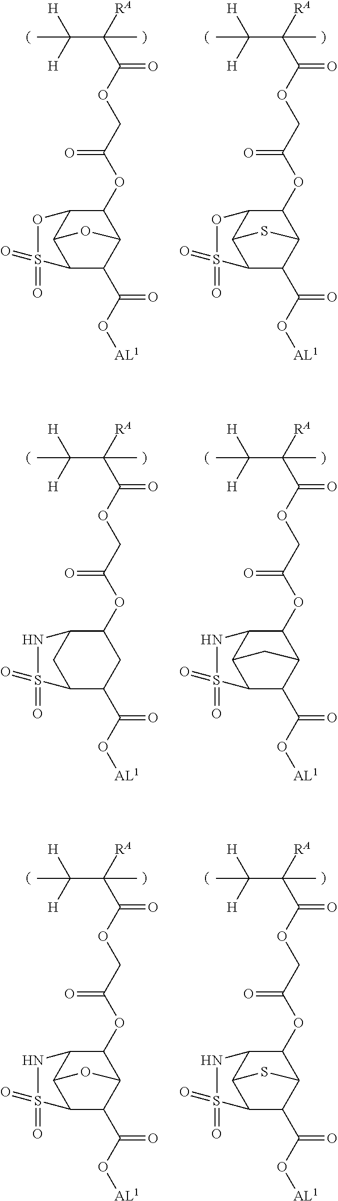

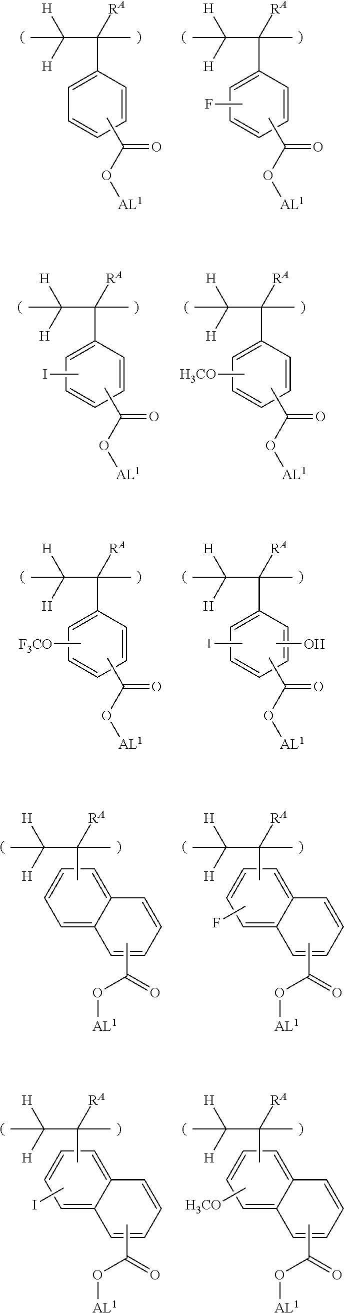

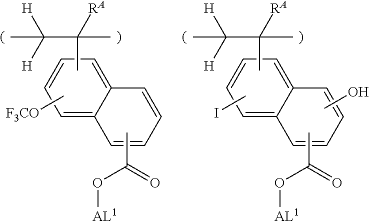

Specific examples of the repeat units a2 are shown below, but not limited thereto. In the following formula, R^{A} and AL² are as defined above.

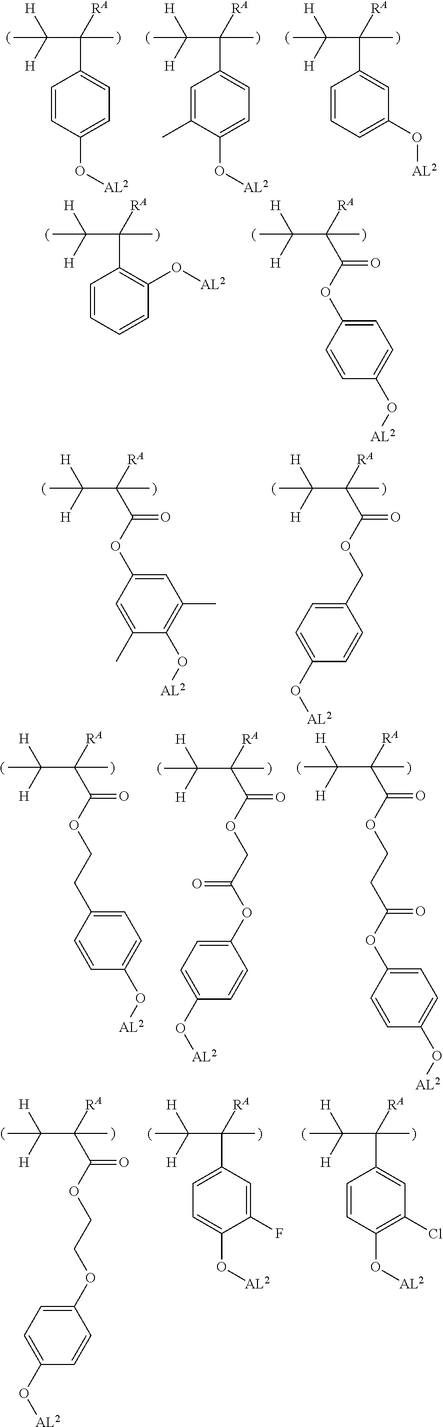

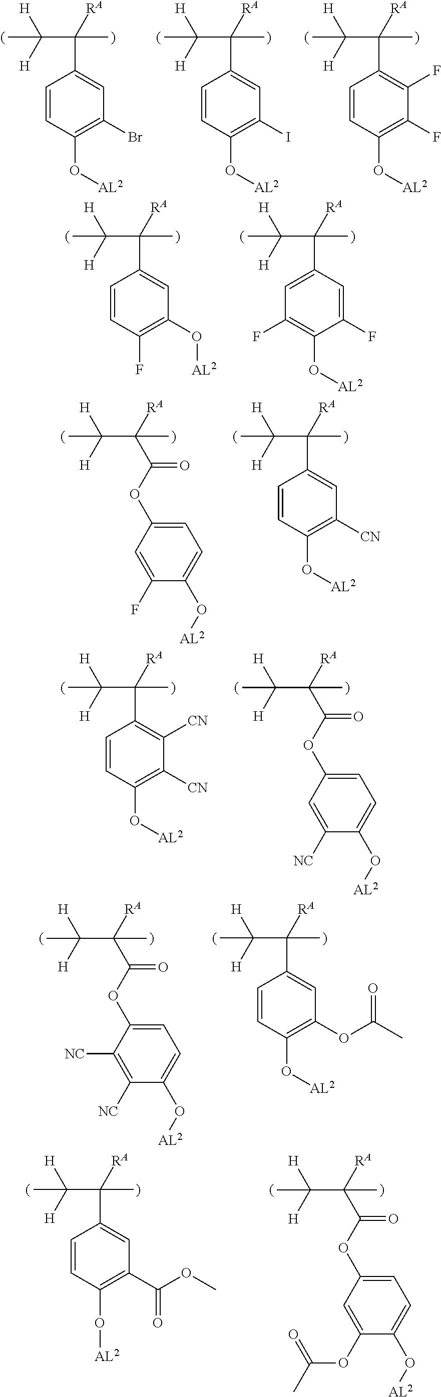

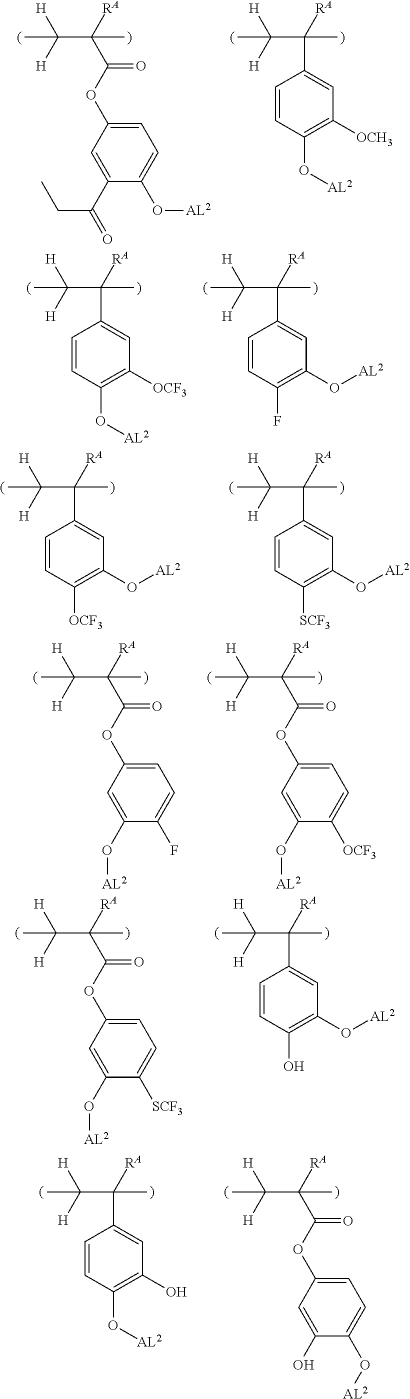

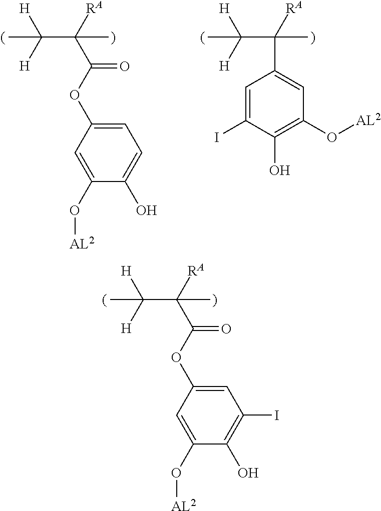

The polymer may comprise repeat units having the following formula (a3)(also referred to as repeat units a3, hereinafter).

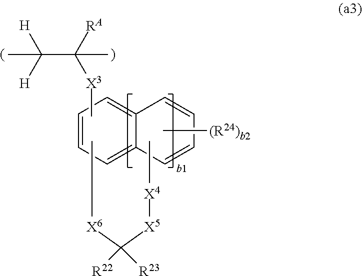

In formula (a3), bl is 0 or 1. When b1 is 0, the relevant structure is a benzene ring, and when b1 is 1, the relevant structure is a naphthalene ring, but from the viewpoint of solvent solubility, the relevant structure is preferably a benzene ring in which bl is 0. b2 is 0, 1, 2, or 3 when b1 is 0, and is 0, 1, 2, 3, 4, or 5 when b1 is 1. From the viewpoint of raw material availability, b2 is preferably 0, 1, 2, or 3 and more preferably 0, 1, or 2.

In formula (a3), R^{A} is a hydrogen atom, a fluorine atom, a methyl group, or a trifluoromethyl group. Of these, a hydrogen atom or a methyl group is preferable, and a hydrogen atom is further preferable.

In formula (a3), X³ is a single bond, *—C(═O)—O—, or *—C(═O)—NH—. * designates a point of attachment to the carbon atom in the backbone. Of these, a single bond or *—C(═O)—O—is preferable, and a single bond is further preferable.

In formula (a3), X⁴ is a single bond, a C₁-C₄ aliphatic hydrocarbylene group, a carbonyl group, a sulfonyl group, or groups obtained by combining these. Of these, from the viewpoint of raw material availability, a single bond, a carbonyl group, or a sulfonyl group is preferable, and from the viewpoint of a polar group generated after the reaction, a single bond or a carbonyl group is more preferable.

In formula (a3), X⁵ and X⁶ are each independently an oxygen atom or a sulfur atom. Provided that X⁴ and X⁶ bond to adjacent carbon atoms of an aromatic ring. X⁵ and X⁶ may be the same as or different from each other, but X⁵ and X⁶ are both preferably an oxygen atom from the viewpoint of reactivity.

In formula (a3), R²² and R²³ are each independently a hydrogen atom, or a C₁-C₂₀ hydrocarbyl group which may contain a heteroatom. The hydrocarbyl group may be saturated or unsaturated, and may be straight, branched, or cyclic. Specific examples thereof include C₁-C₂₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ allyl groups such as a phenyl group and a naphthyl group; C₇-C₂₀ aralkyl groups such as a benzyl group, a 1-phenylethyl group, and a 2-phenylethyl group; and groups obtained by combining these. Some or all of the hydrogen atoms in the hydrocarbyl group may be substituted with a group containing a heteroatom such as an oxygen atom, a sulfur atom, a nitrogen atom, or a halogen atom, some of —CH₂— in the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

R²² and R²³ may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring may be substituted with a group containing a heteroatom such as an oxygen atom, a sulfur atom, a nitrogen atom, or a halogen atom, some of —CH₂— in the ring may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

In formula (a3), R²⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group. The halogen atom is preferably a fluorine atom, a chlorine atom, a bromine atom, or an iodine atom, and is more preferably a fluorine atom or an iodine atom. The hydrocarbyl moiety of the hydrocarbyl group, the hydrocarbyloxy group, the hydrocarbyloxycarbonyl group, and the hydrocarbylthio group may be saturated or unsaturated and straight, branched, or cyclic. Specific examples thereof are as exemplified above for the hydrocarbyl group represented by R²² and R²³. Some or all of the hydrogen atoms in the hydrocarbyl group may be substituted with a group containing a heteroatom such as an oxygen atom, a sulfur atom, a nitrogen atom, or a halogen atom, some of —CH₂—in the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. When b2 is 2, respective R²⁴ may be the same as or different from each other.

When b2 is 2 or more, a plurality of R²⁴'s may bond together to form a ring with the carbon atom of the aromatic ring to which they are attached. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring may be substituted with a group containing a heteroatom such as an oxygen atom, a sulfur atom, a nitrogen atom, or a halogen atom, some of—CH₂-in the ring may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

Specific examples of the repeat units a3 are shown below, but not limited thereto. In the following formula. R^{A} is as defined above, and Me is a methyl group. The bonding positions of various substituents on the aromatic ring may be interchanged with each other.

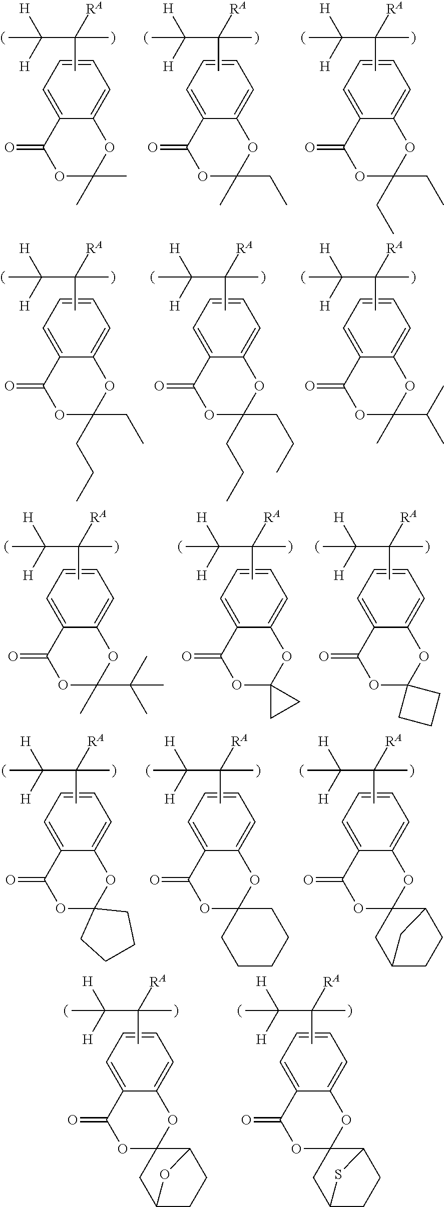

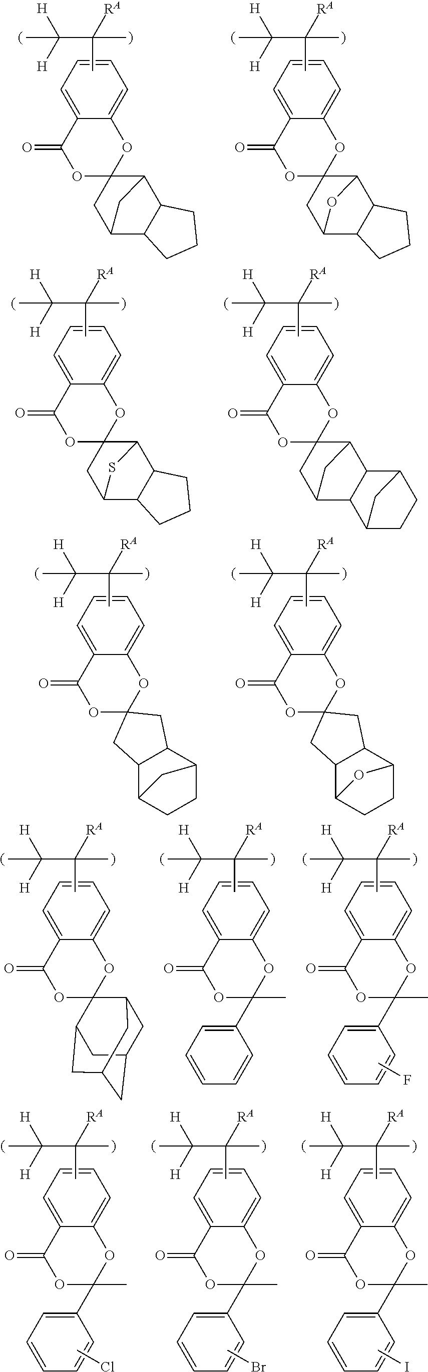

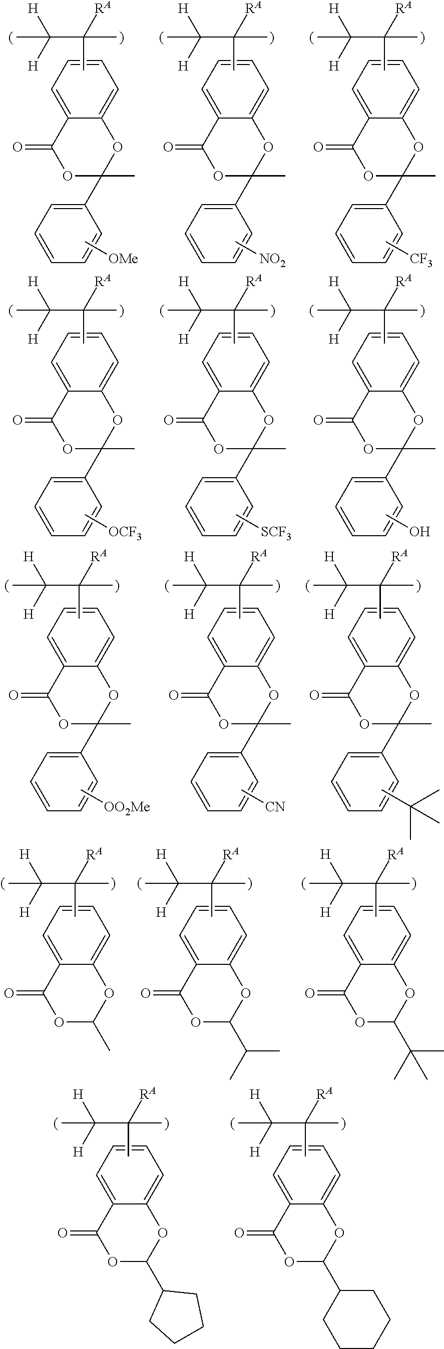

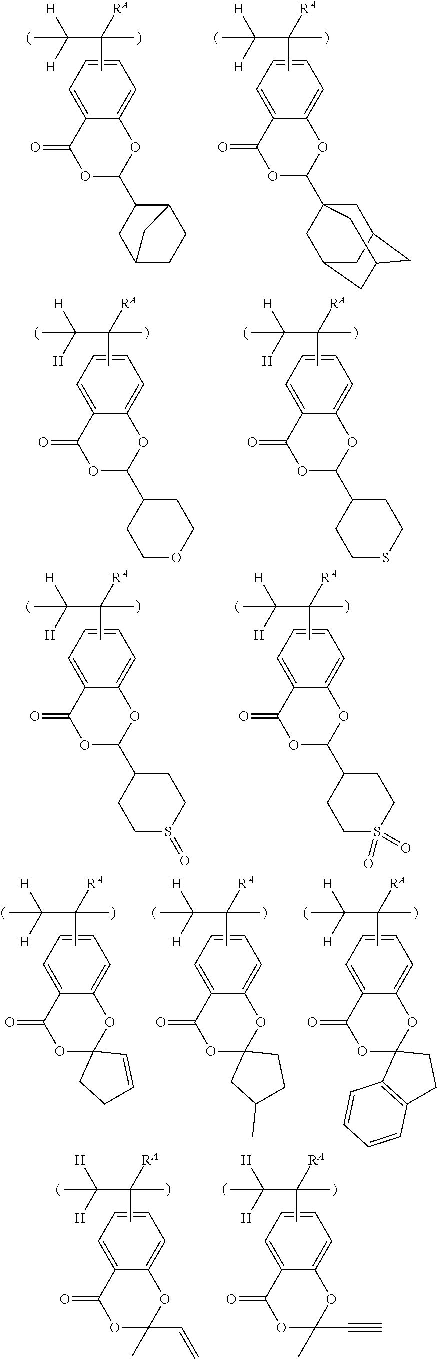

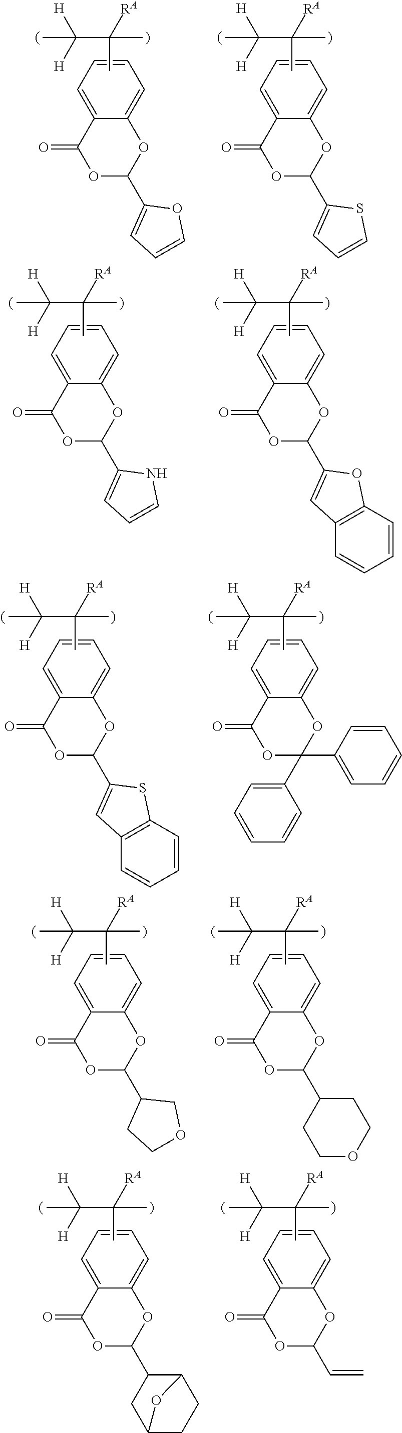

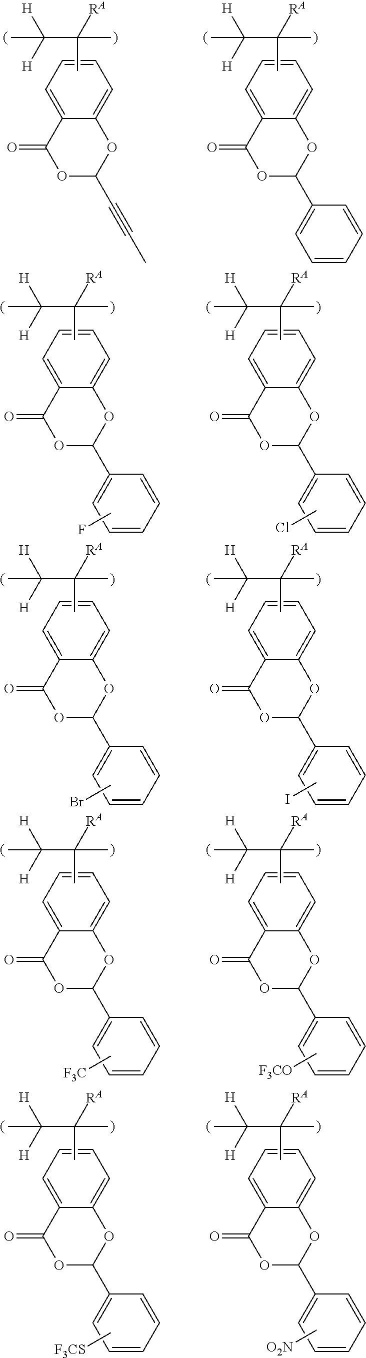

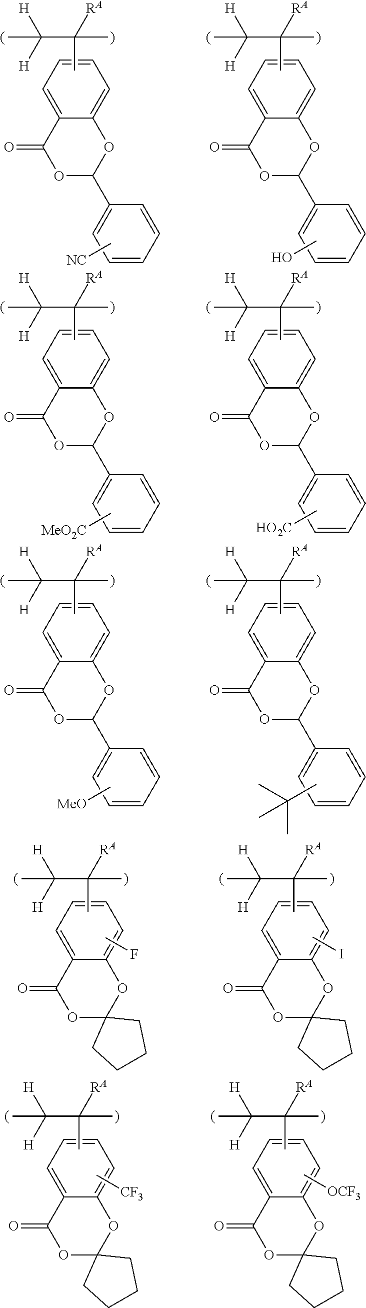

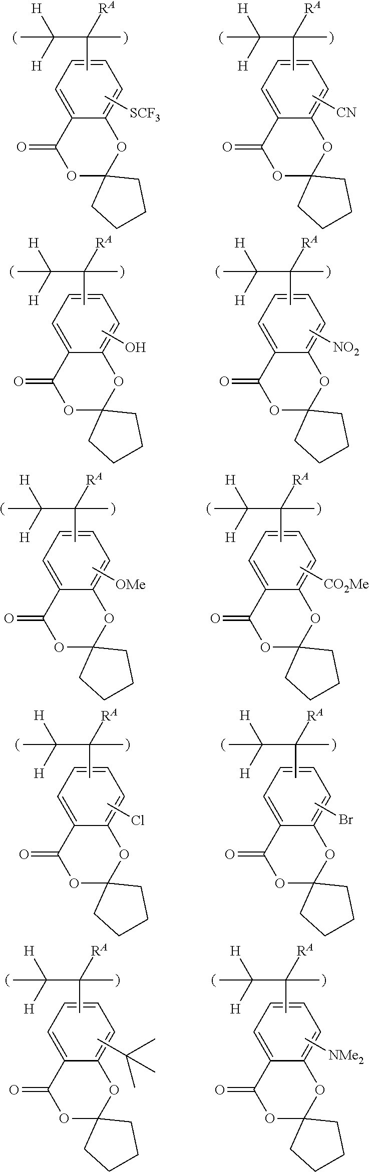

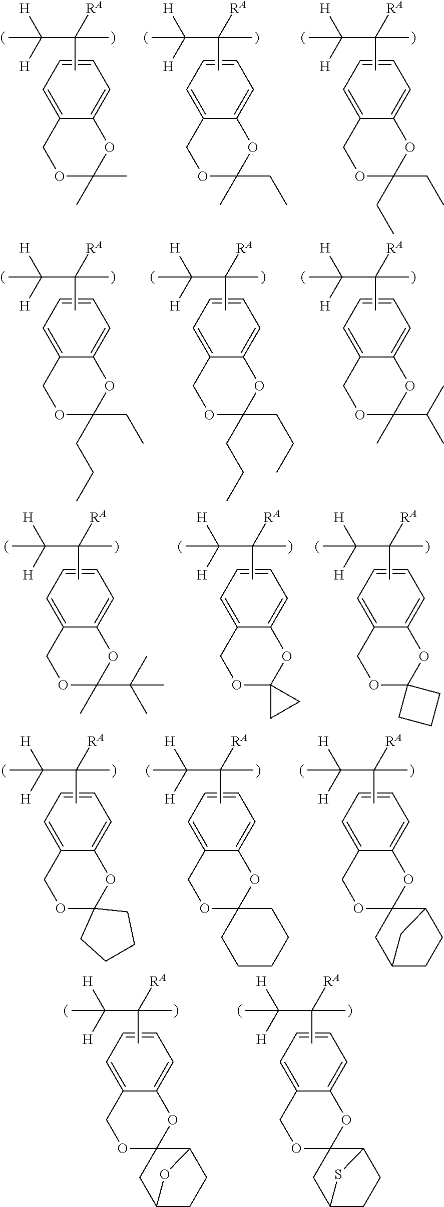

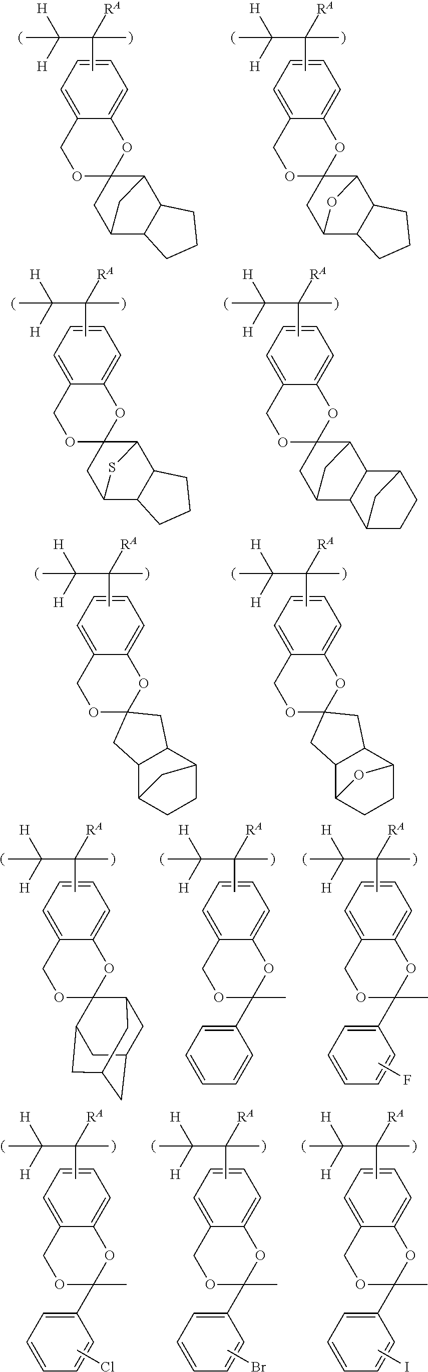

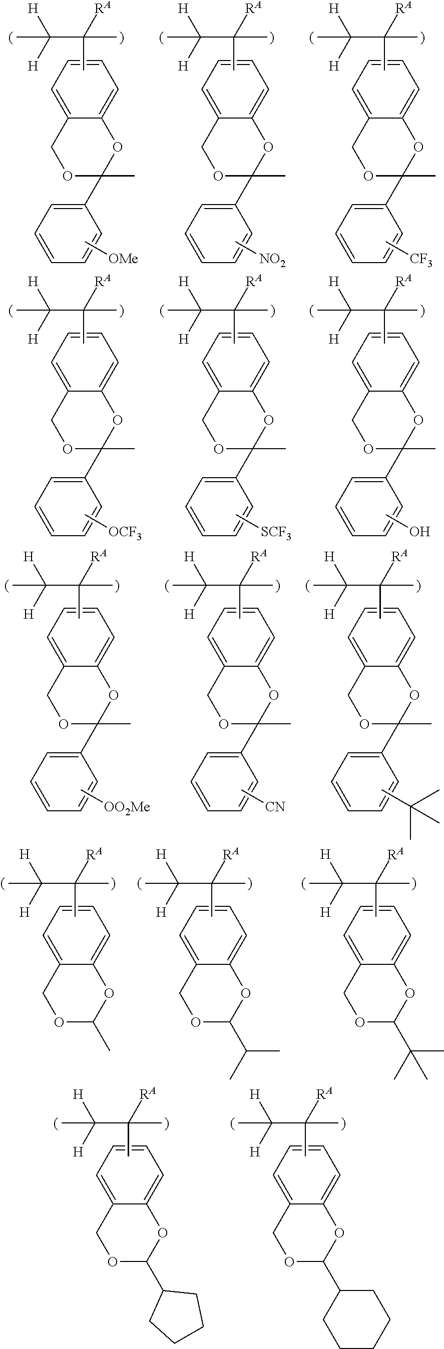

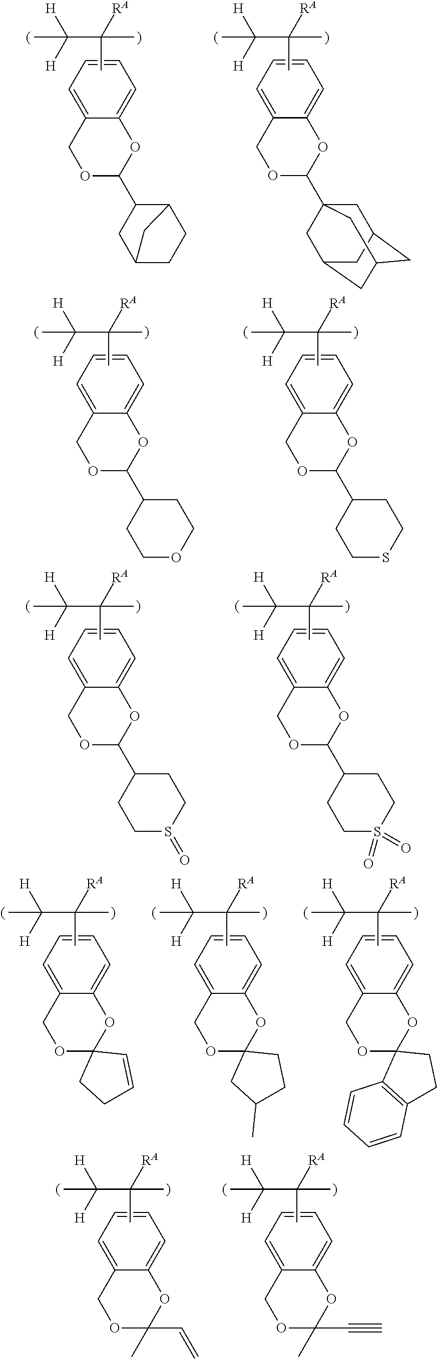

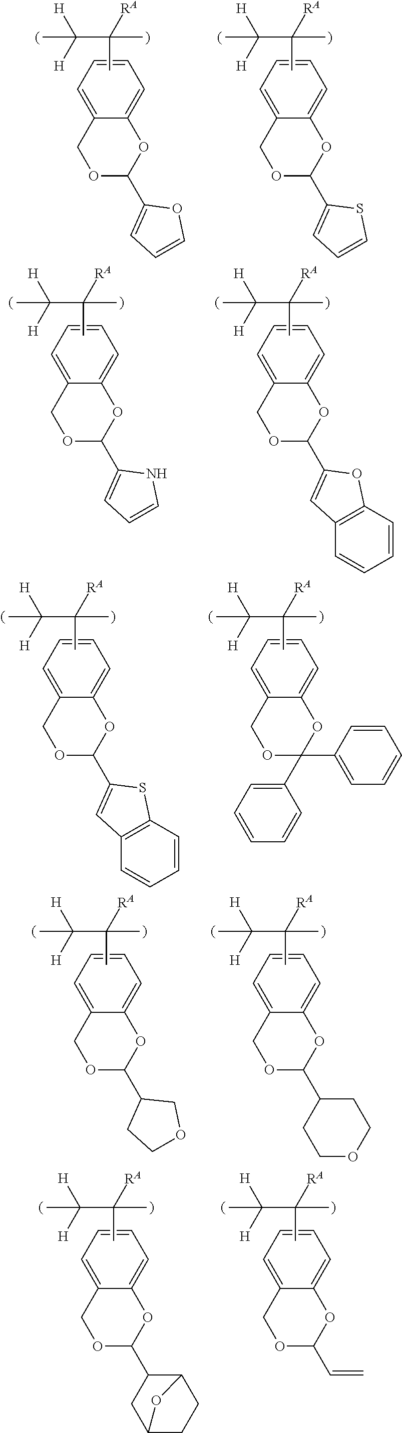

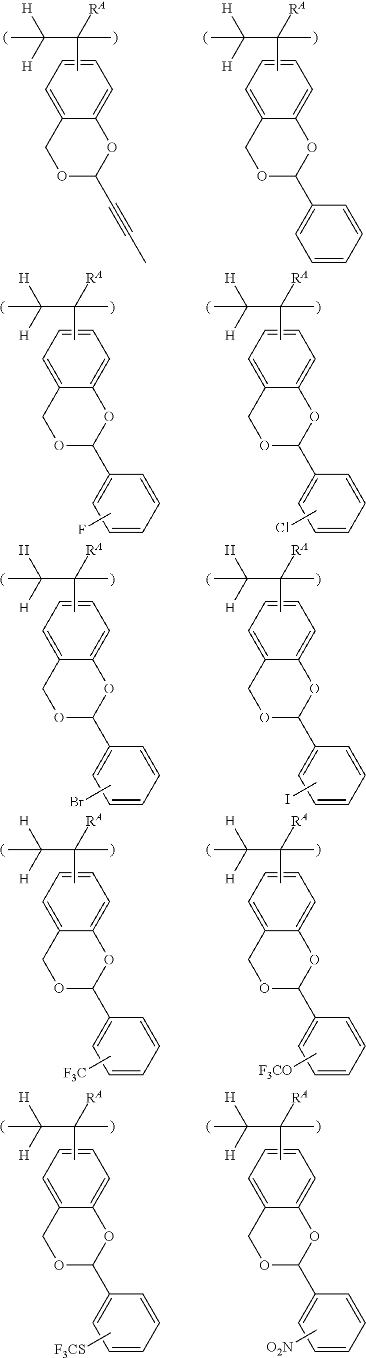

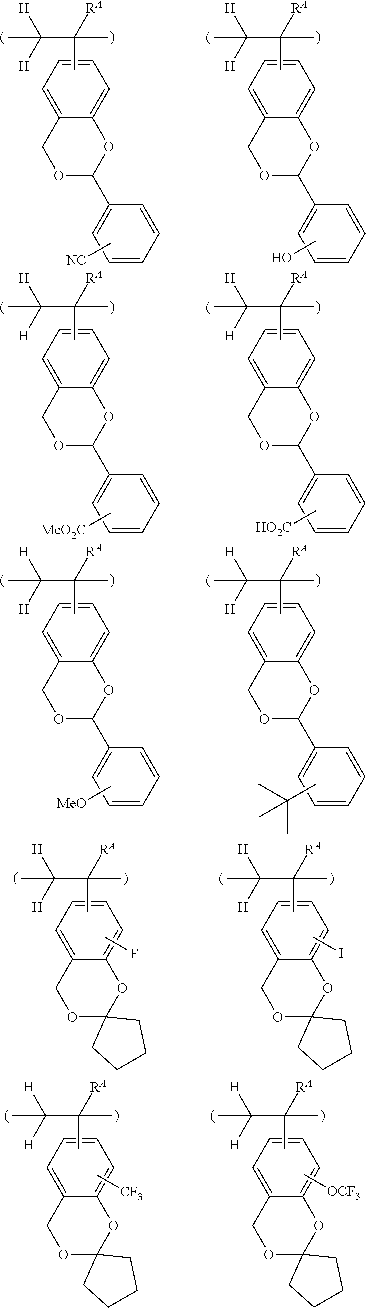

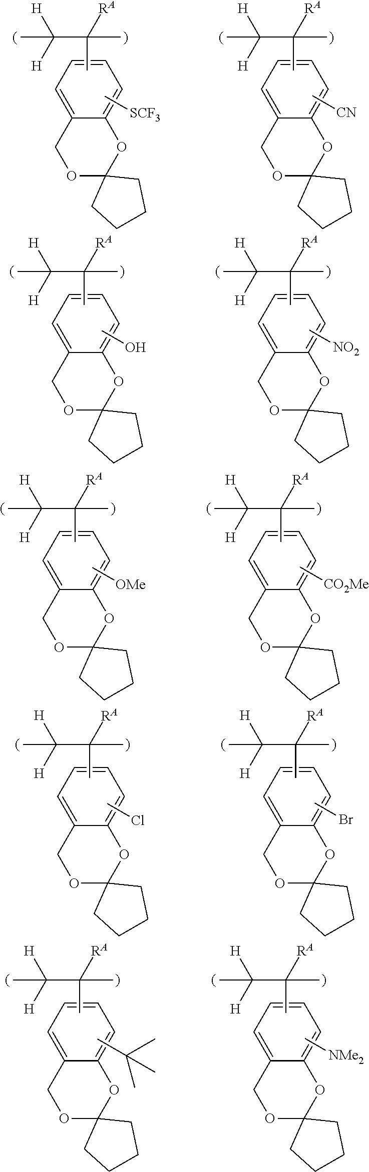

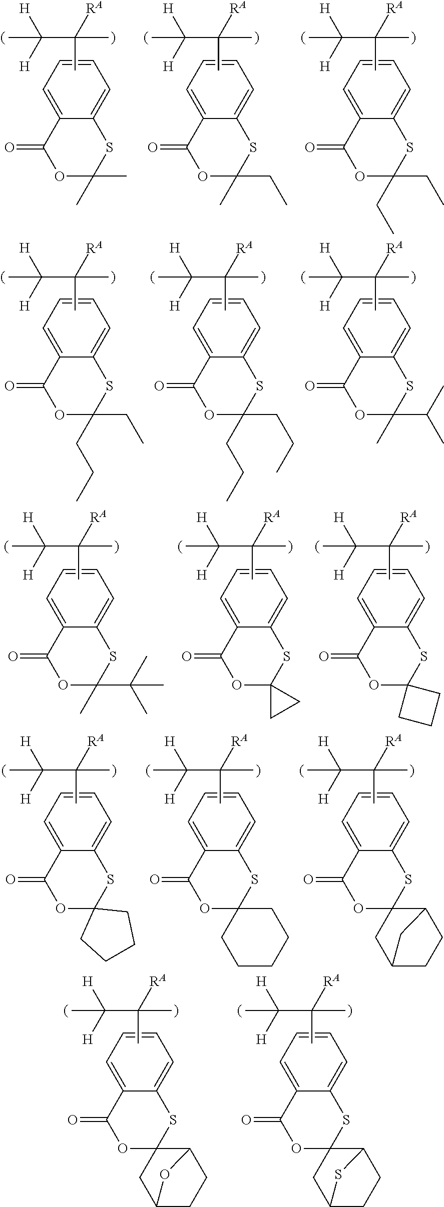

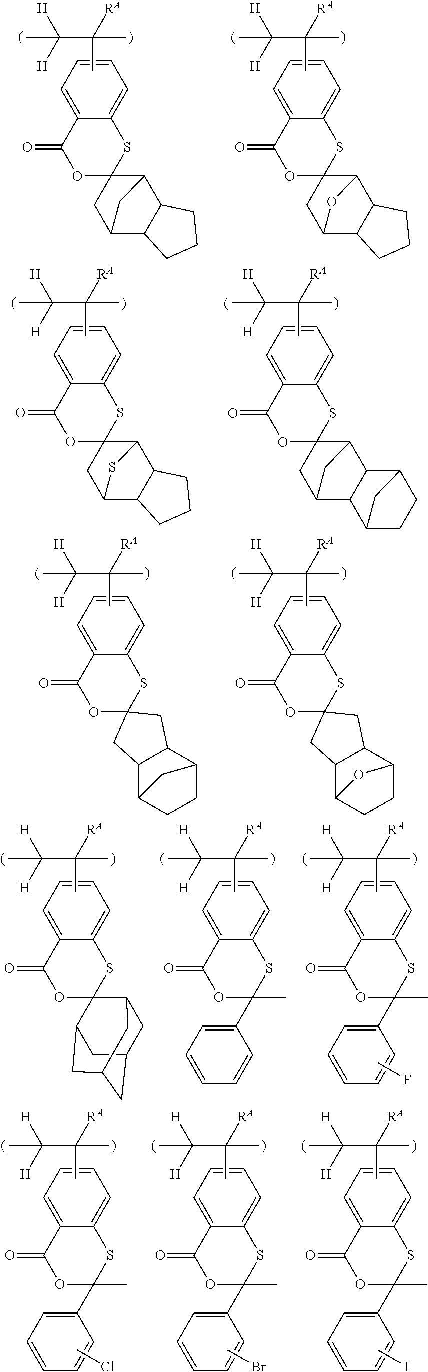

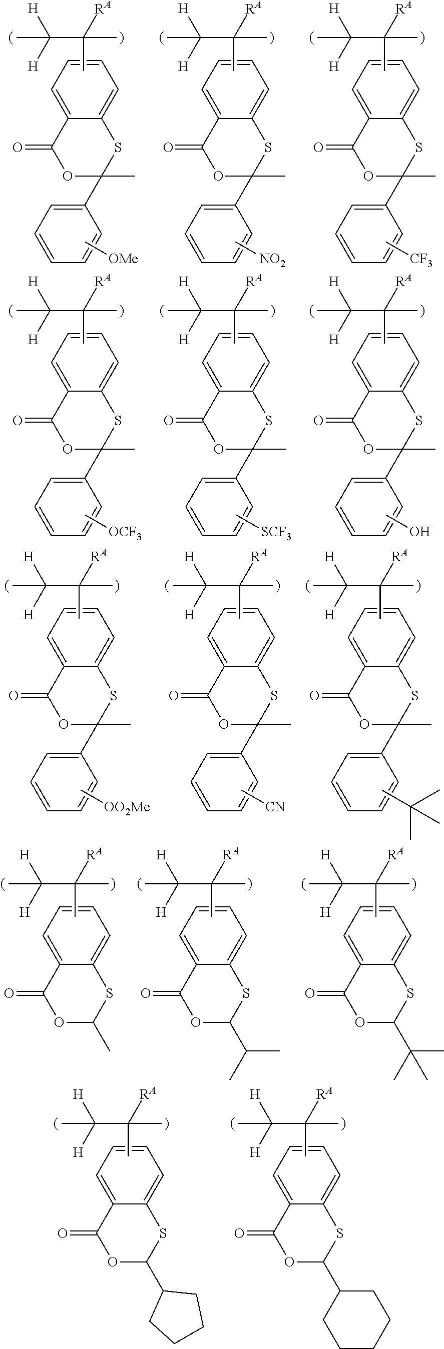

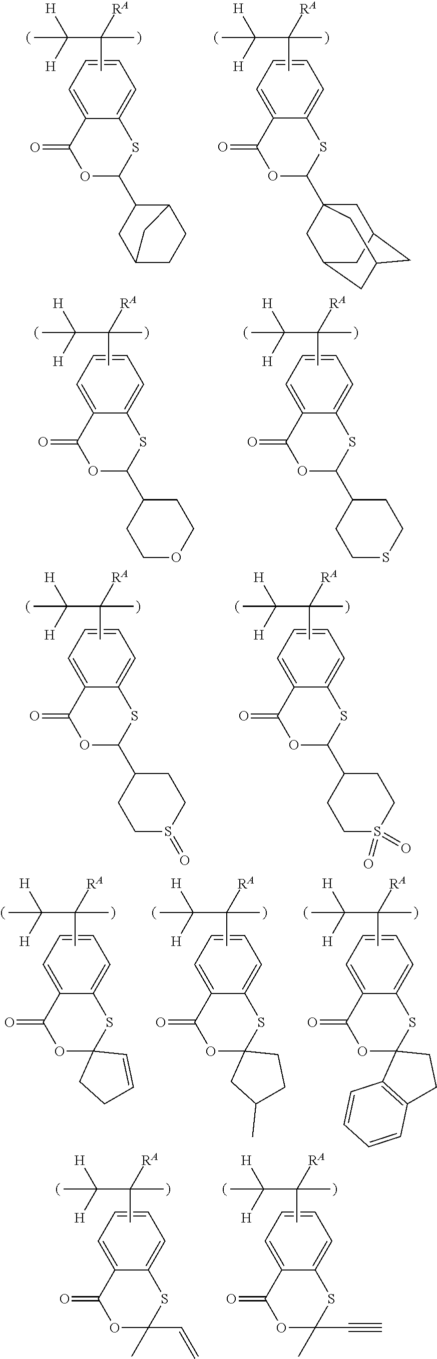

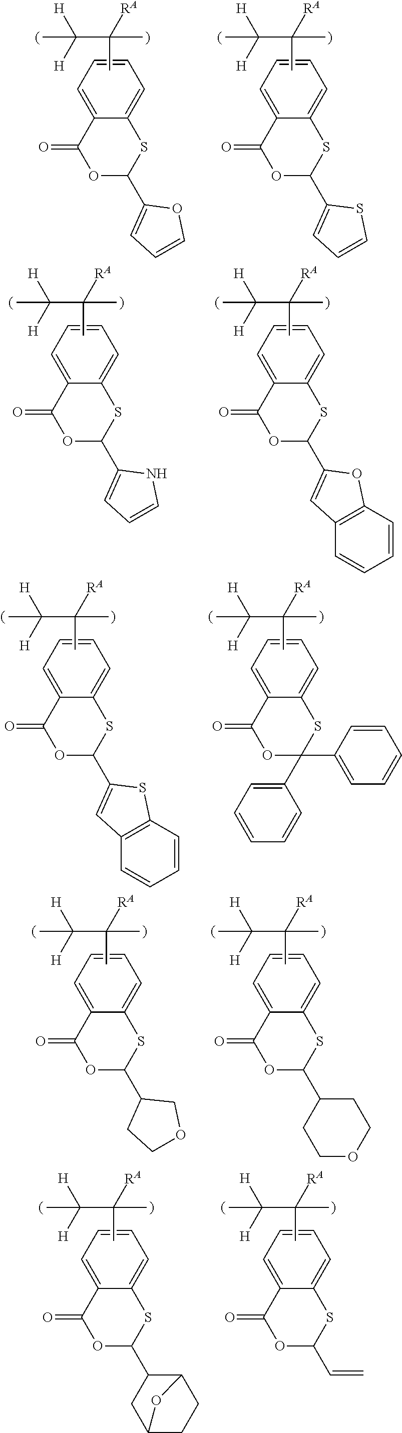

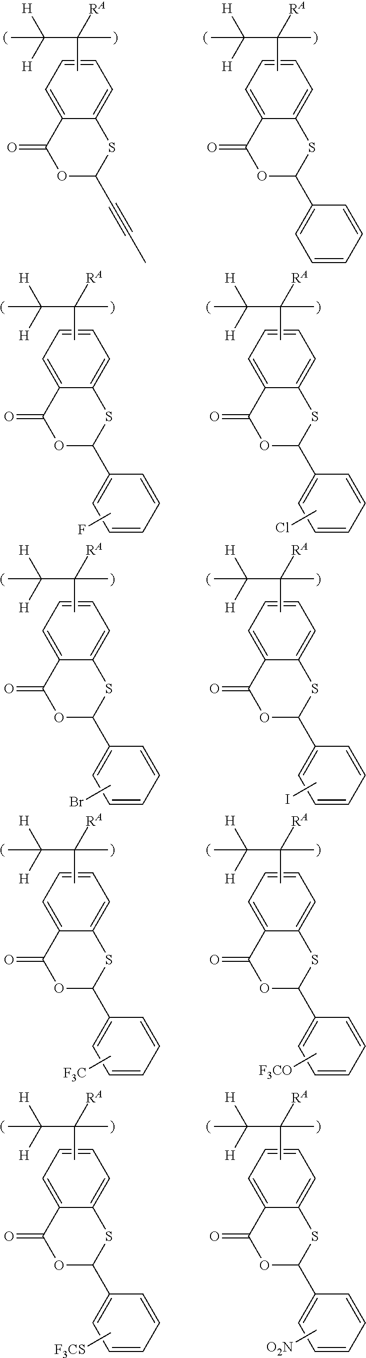

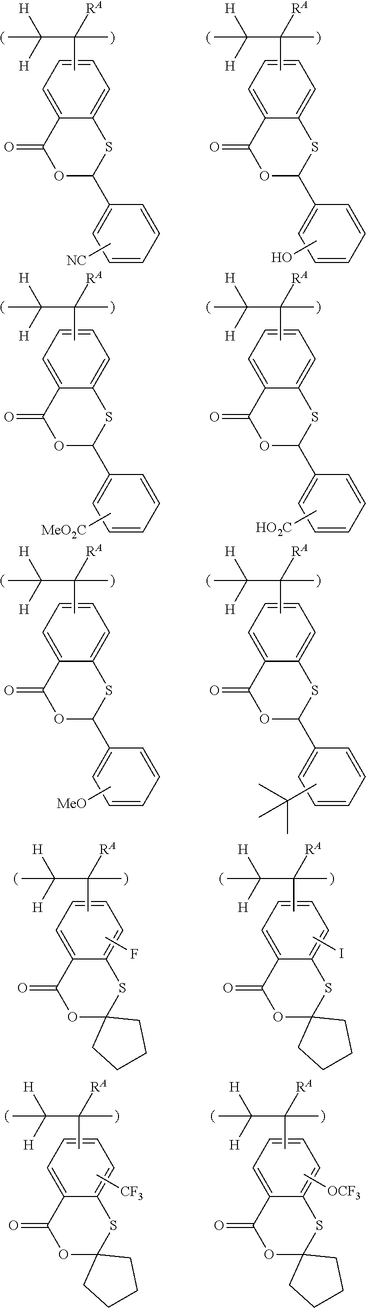

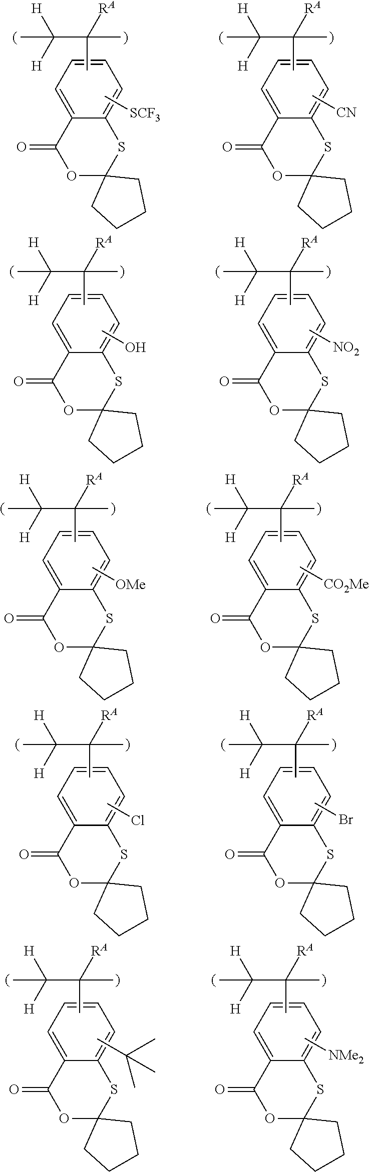

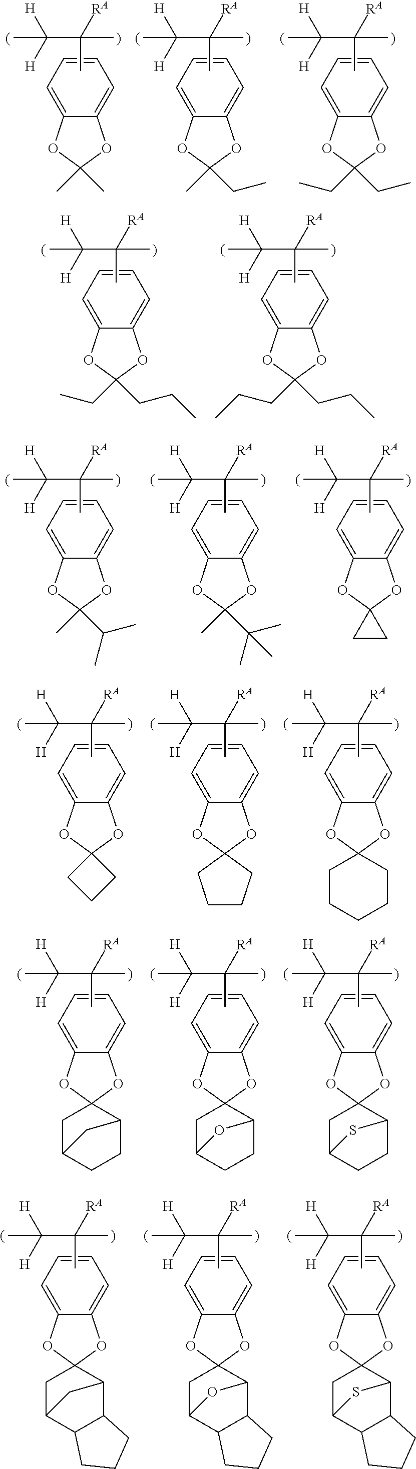

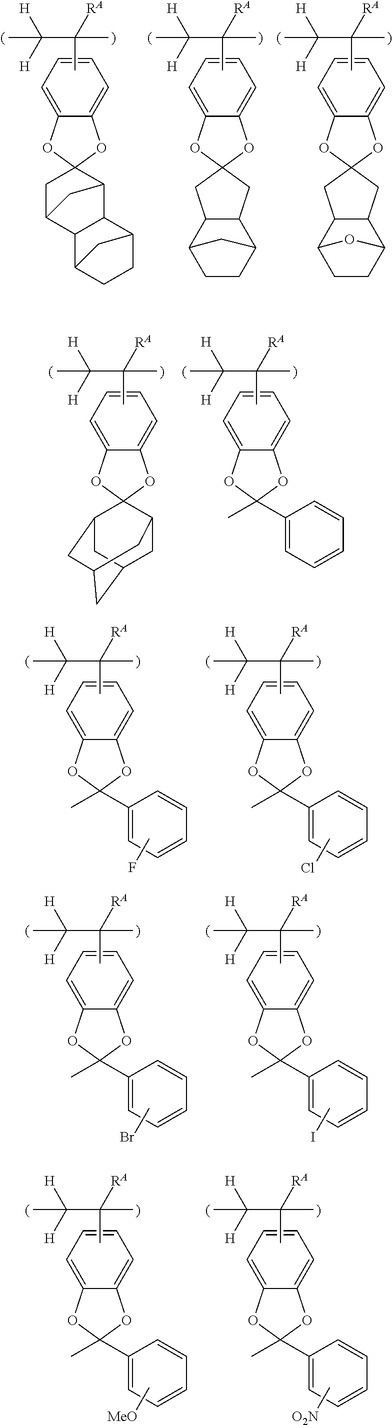

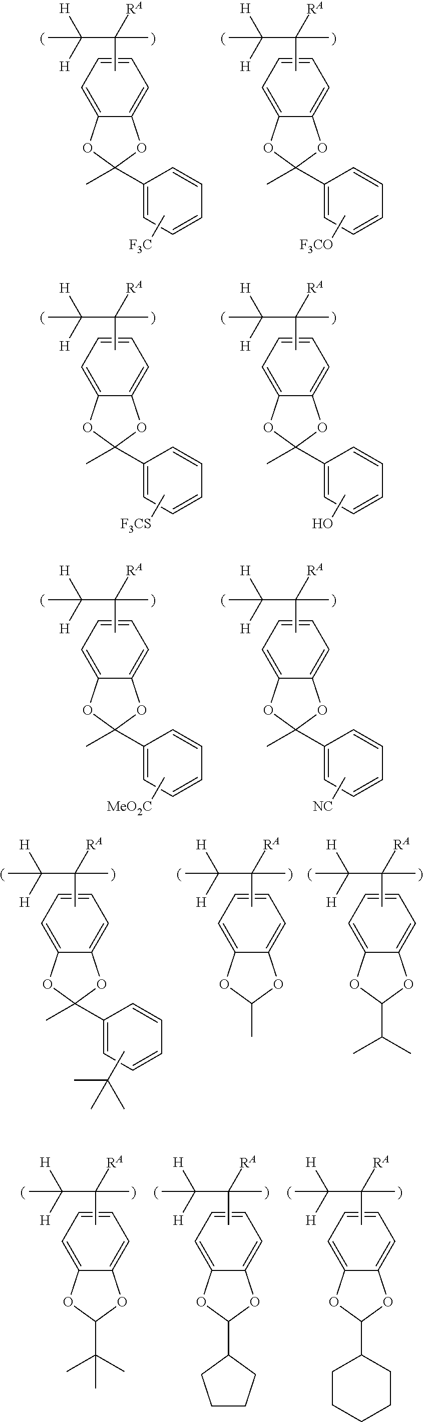

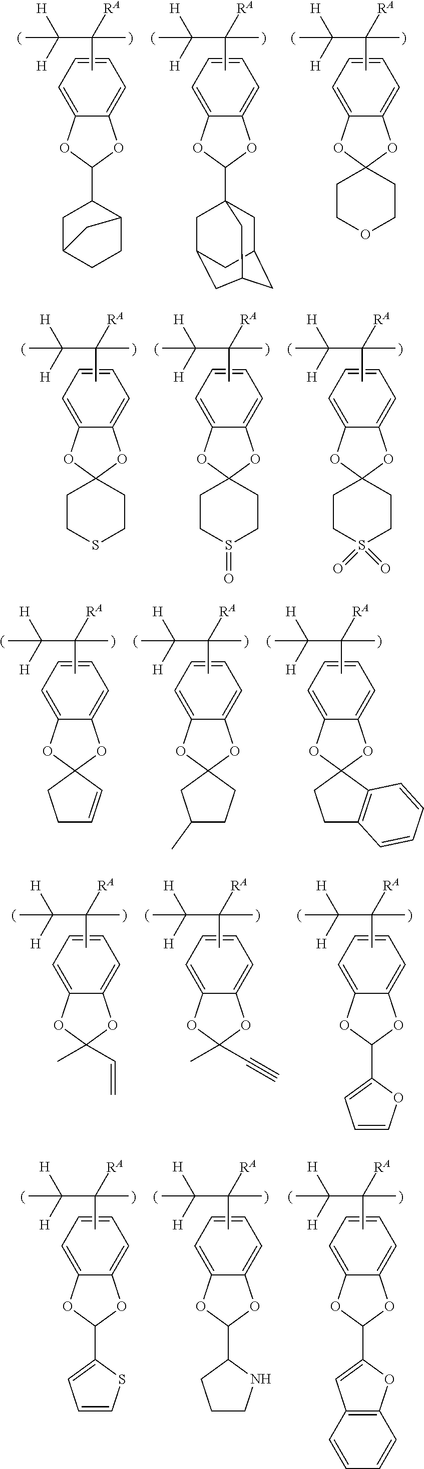

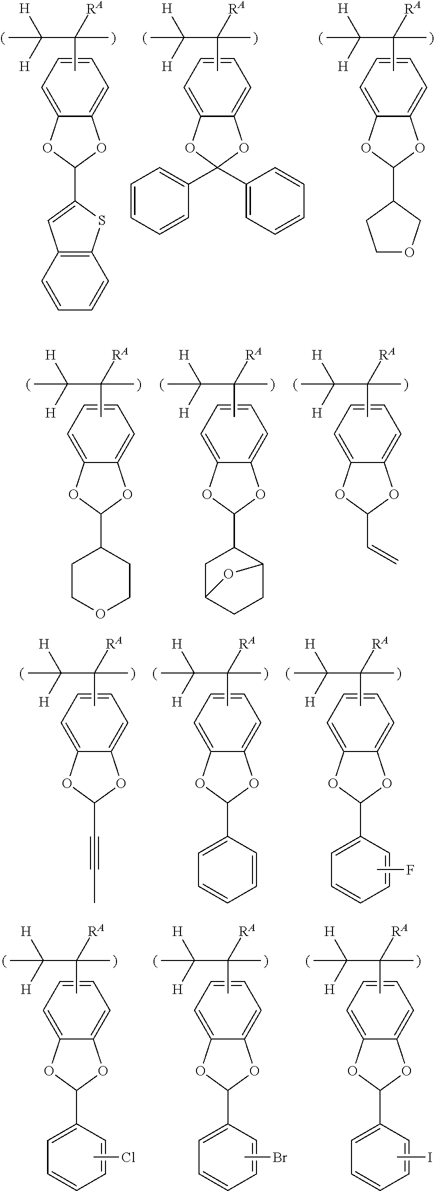

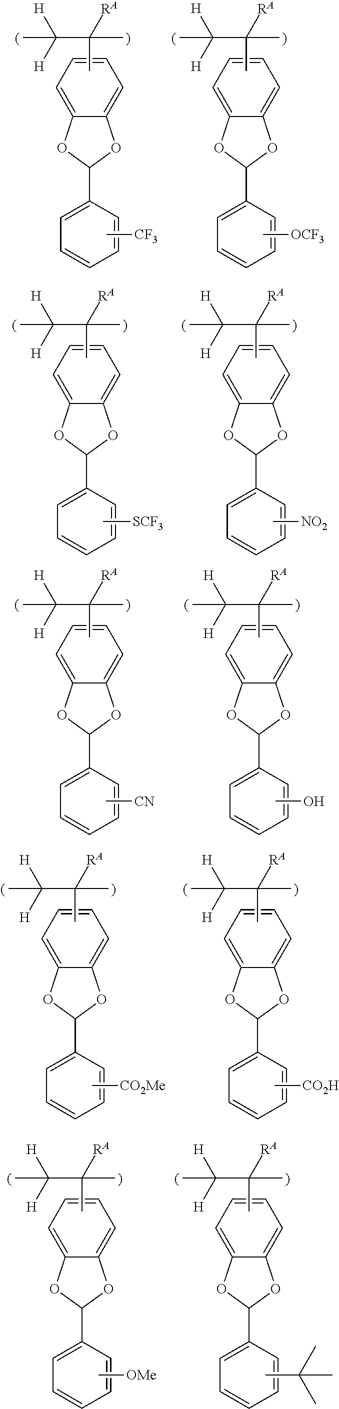

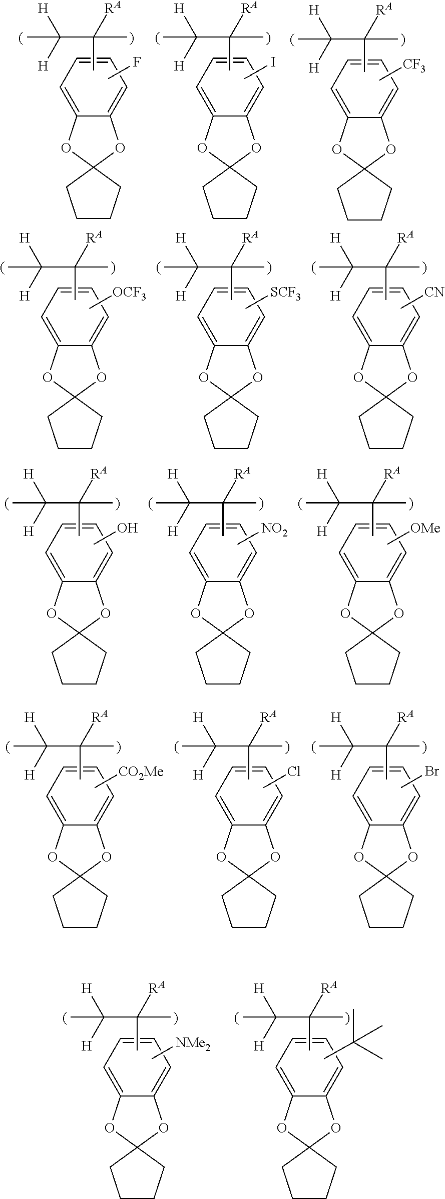

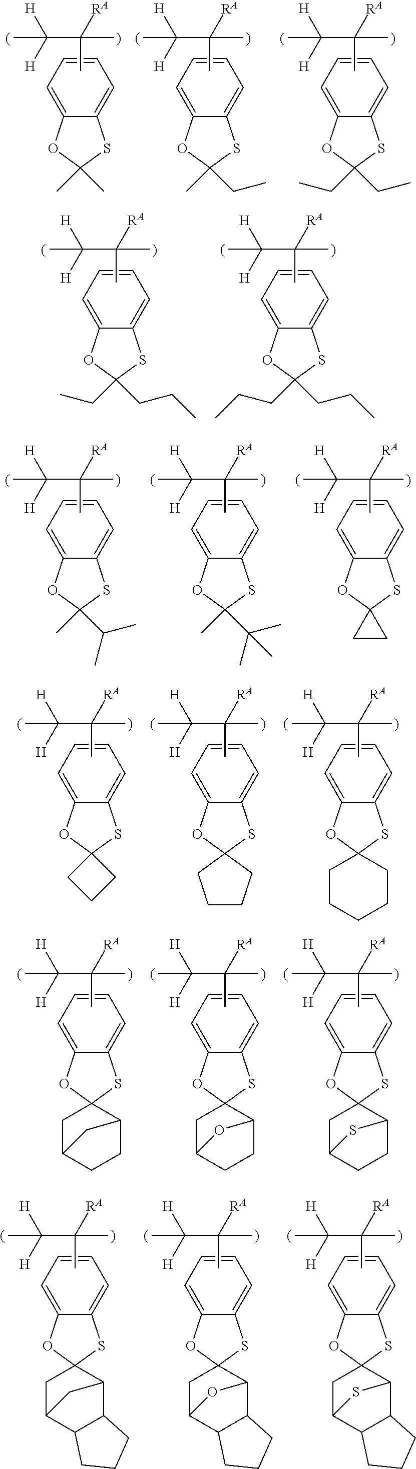

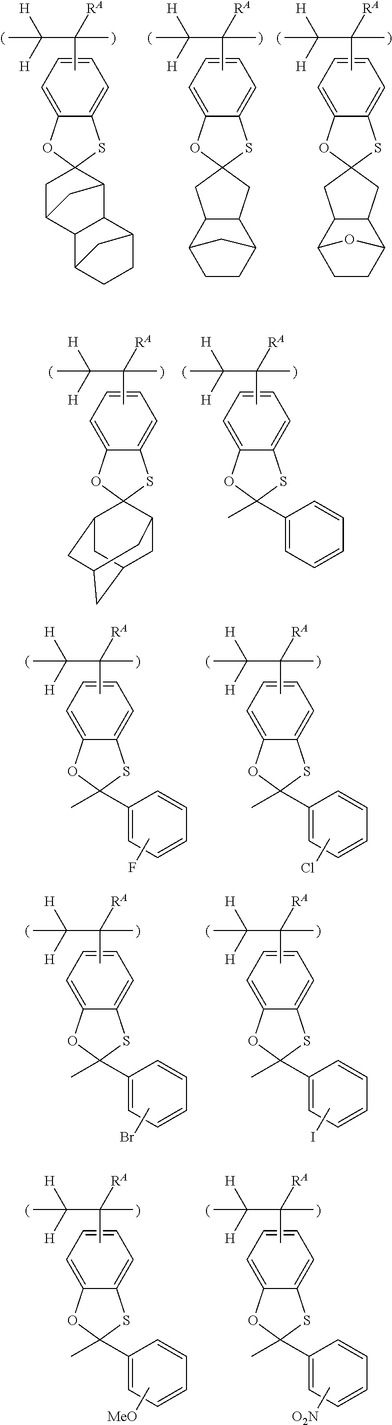

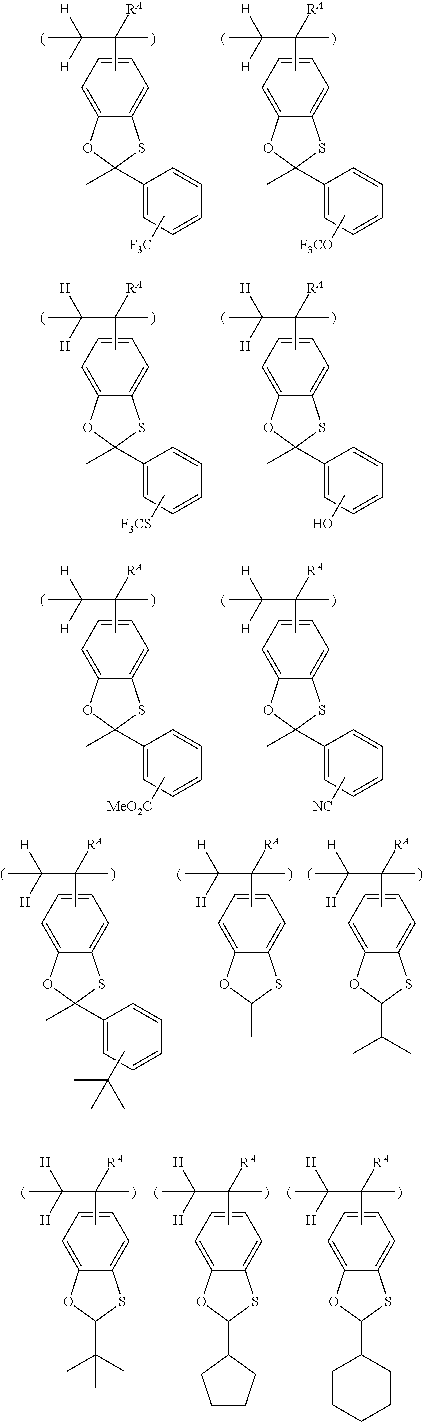

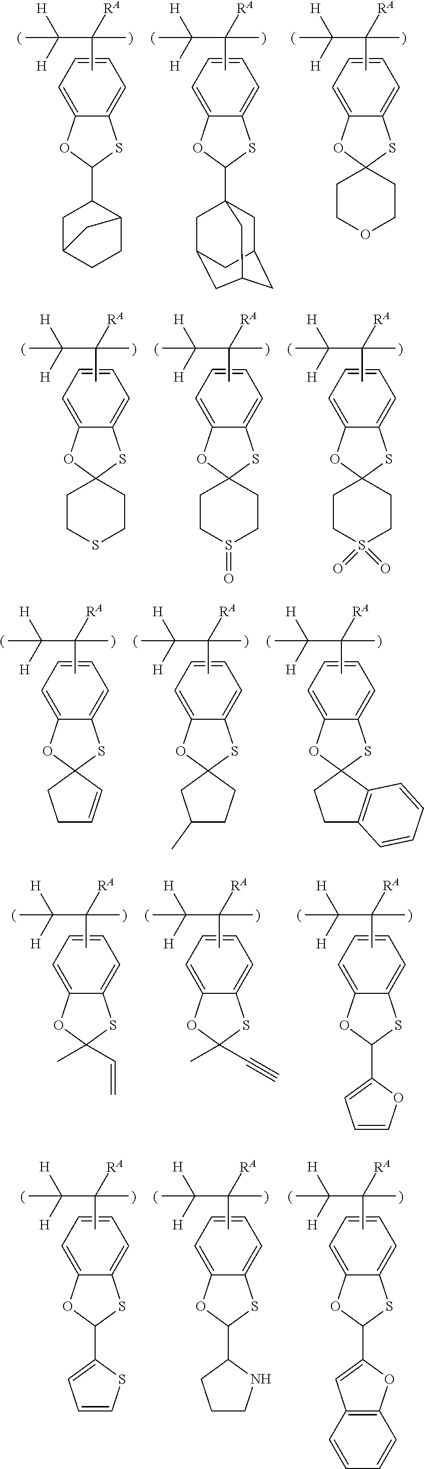

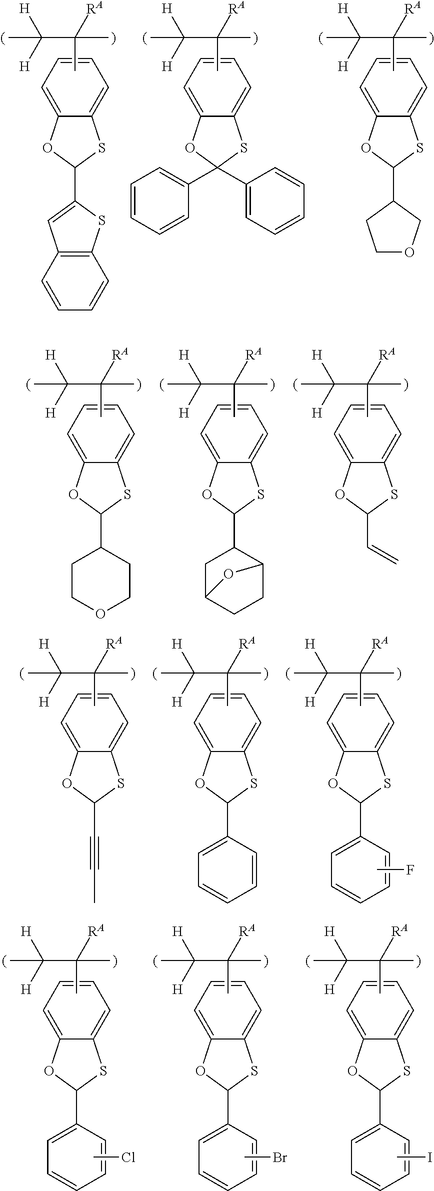

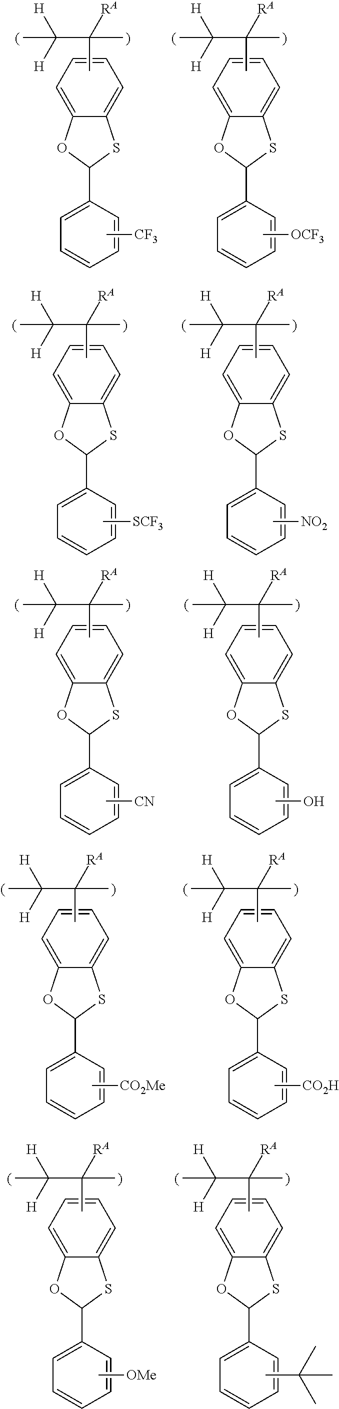

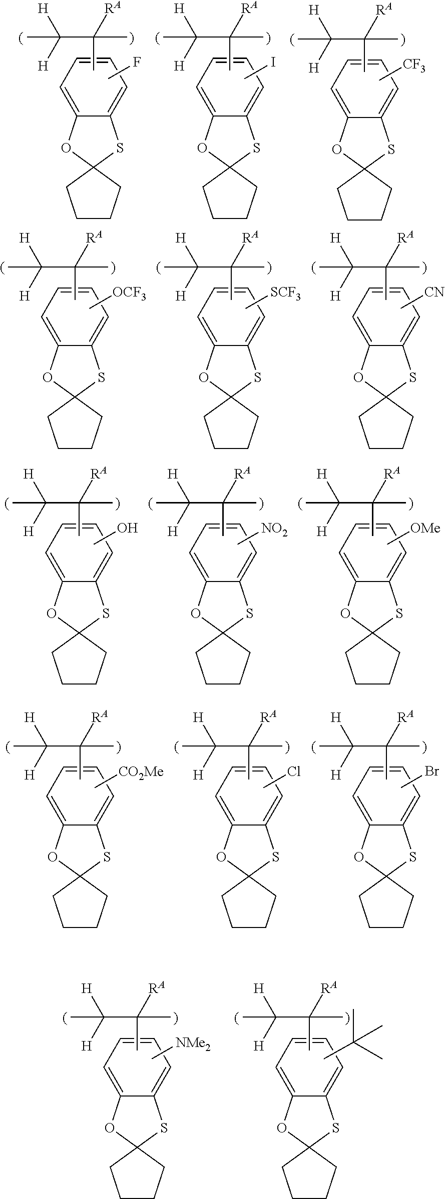

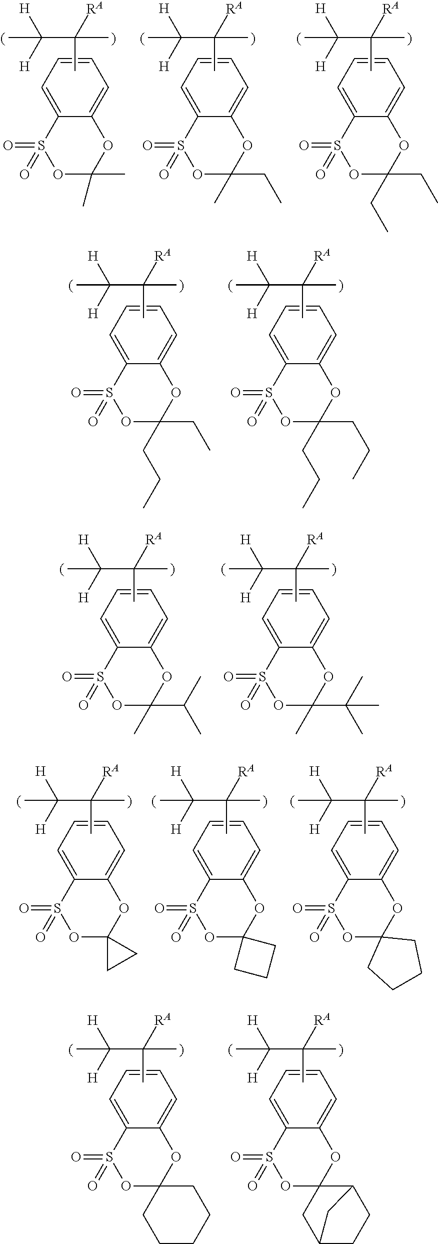

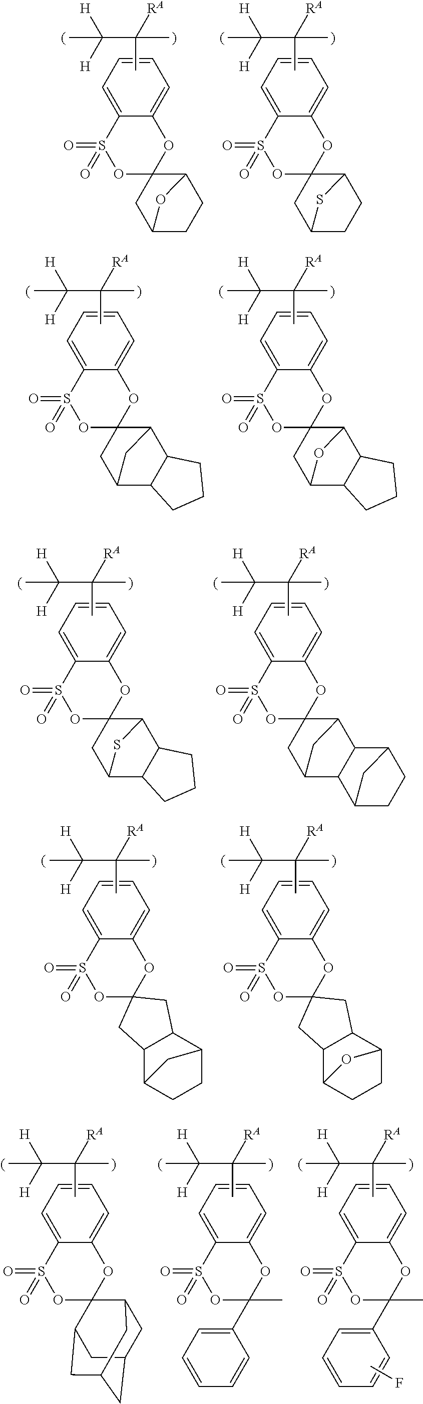

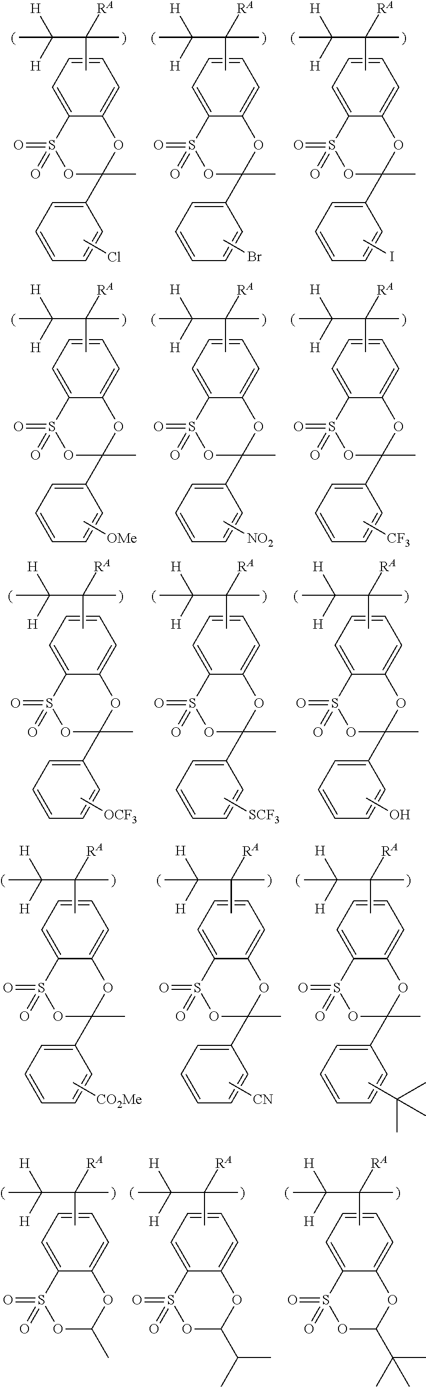

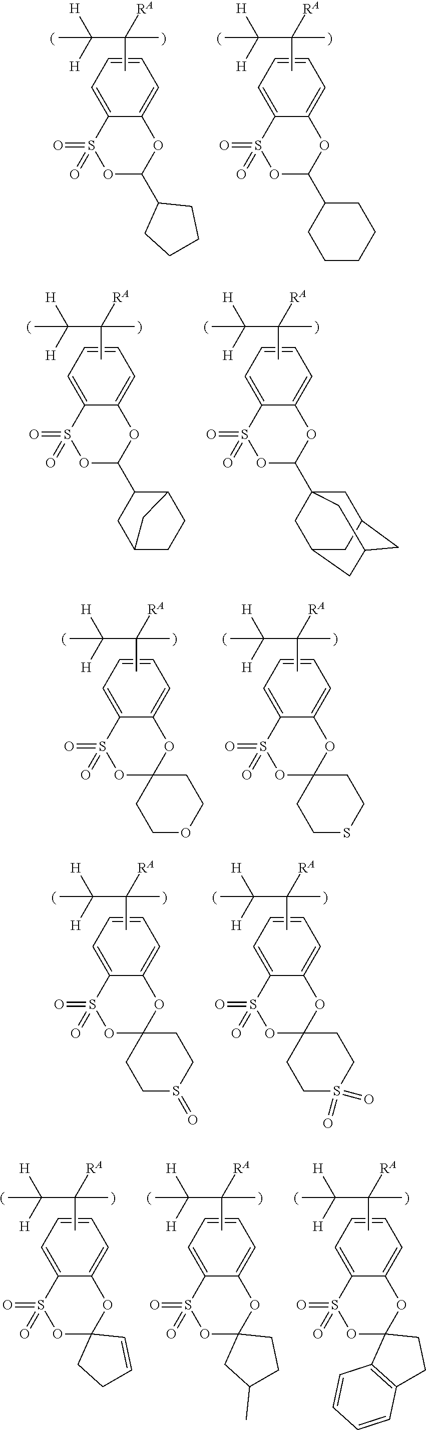

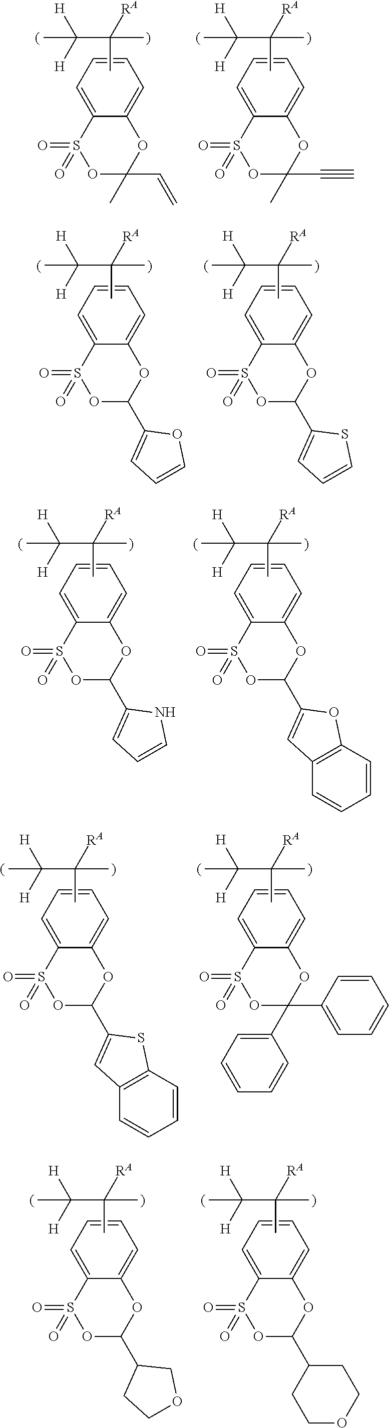

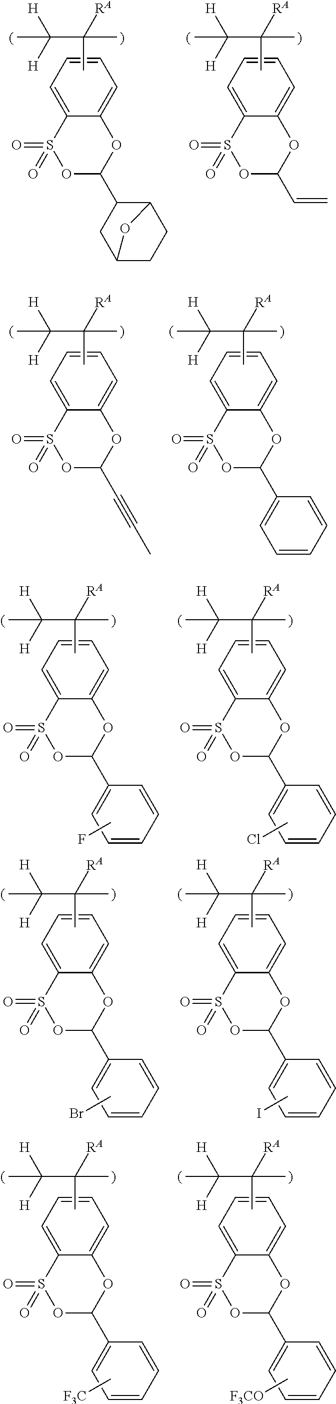

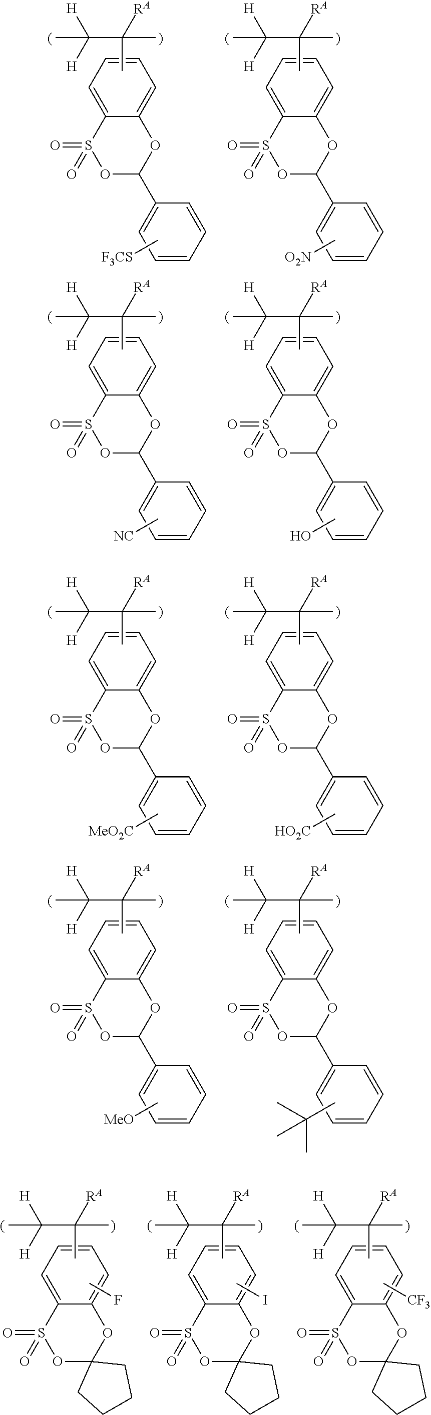

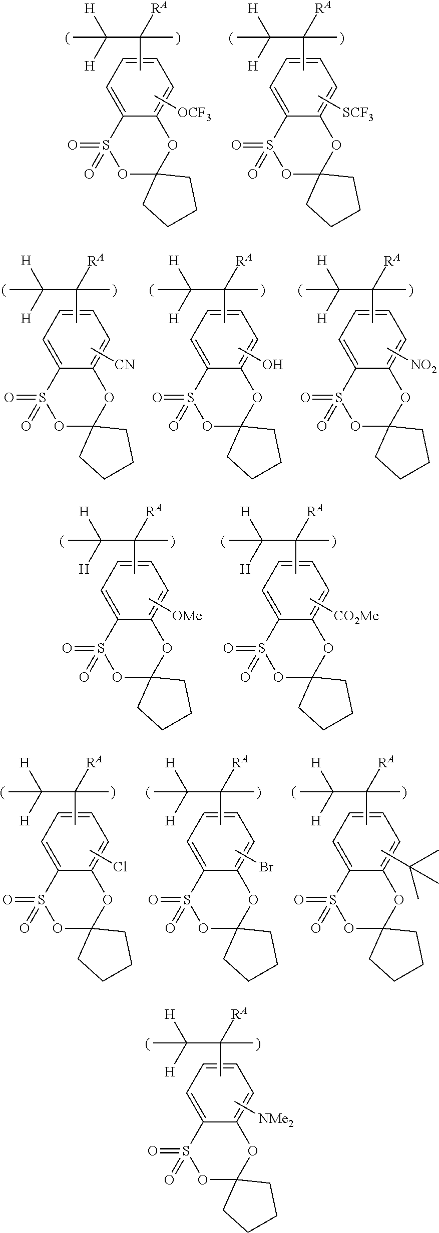

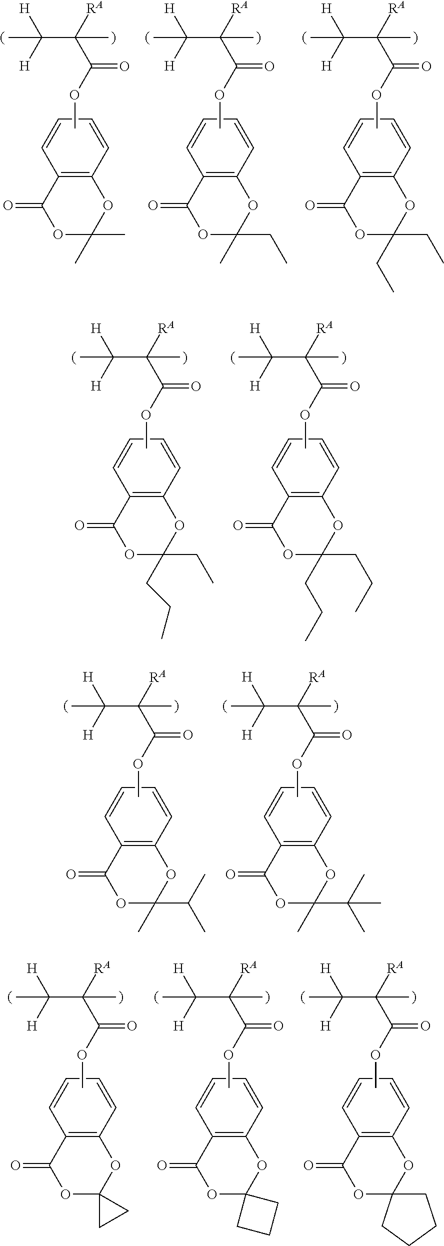

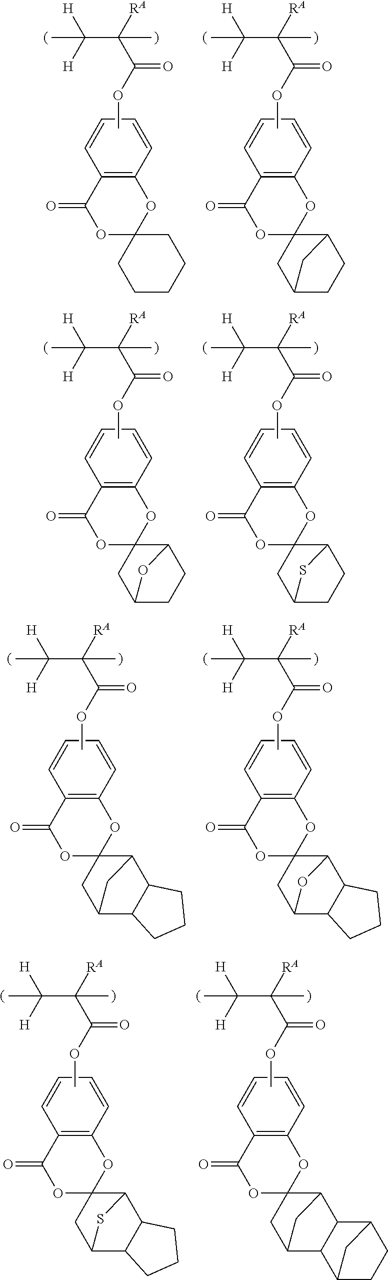

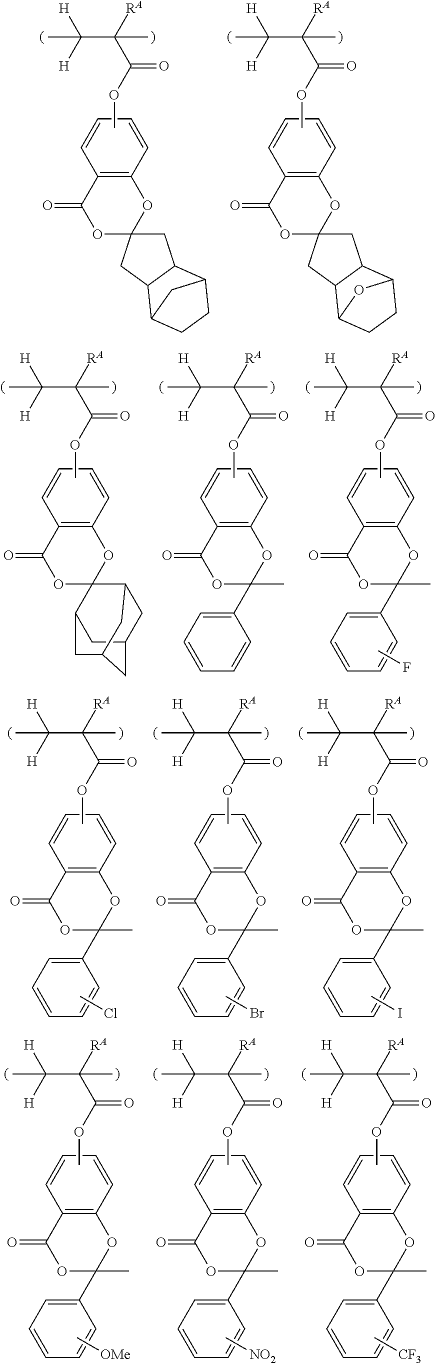

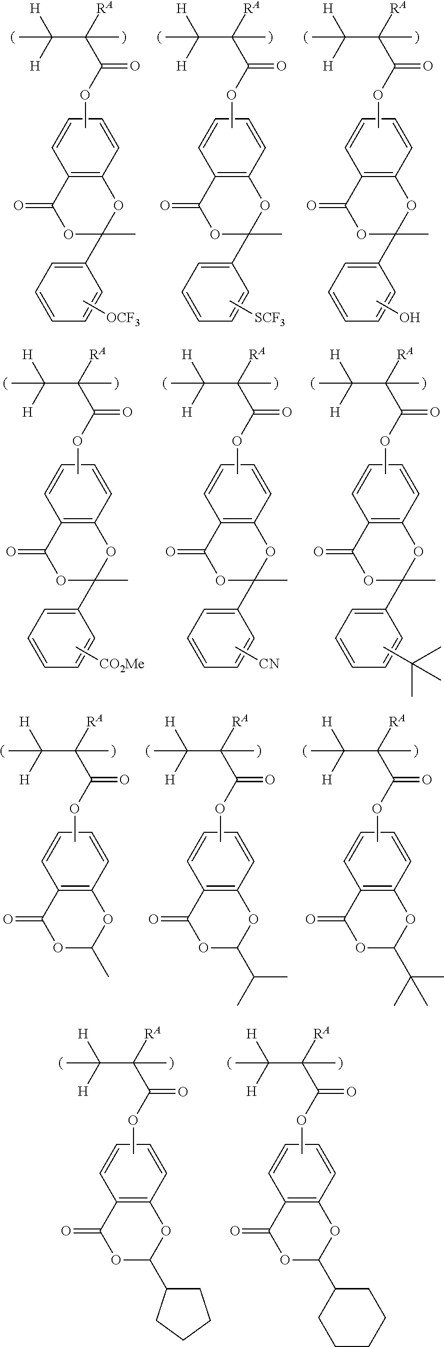

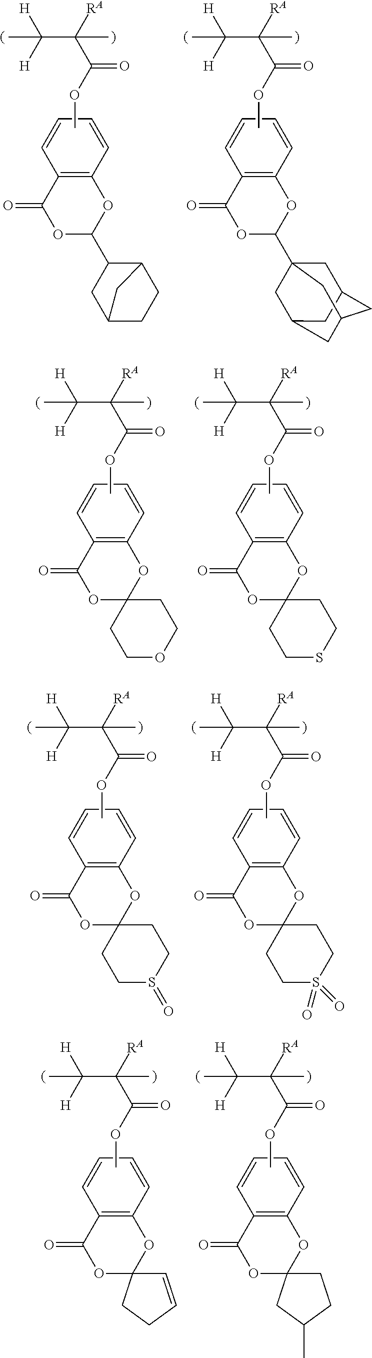

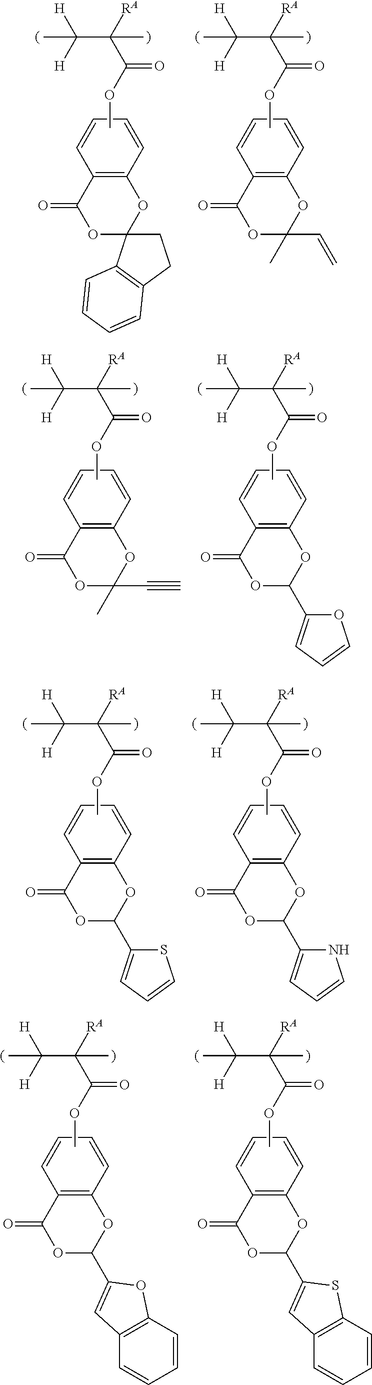

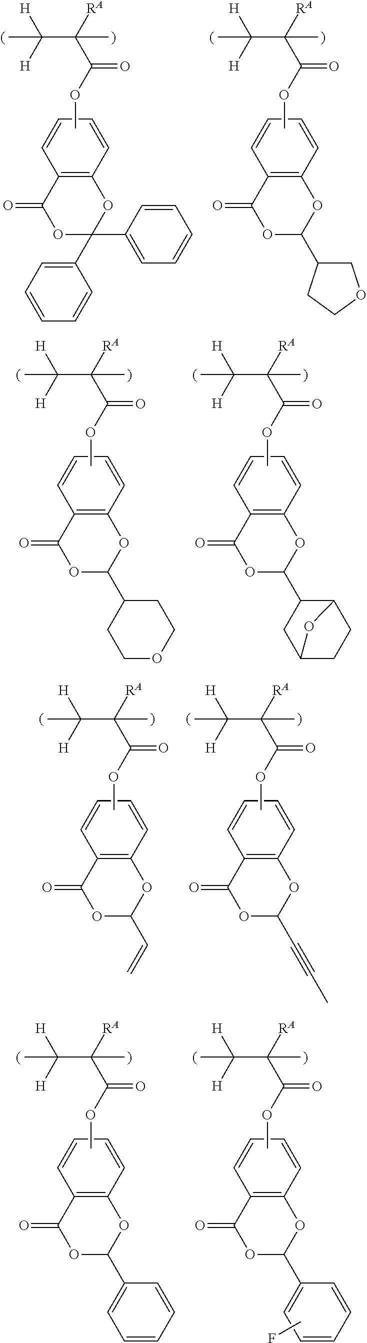

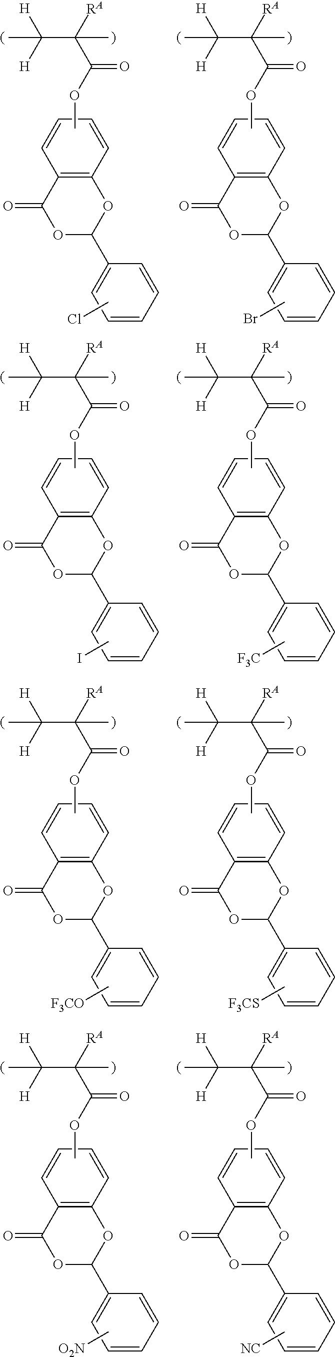

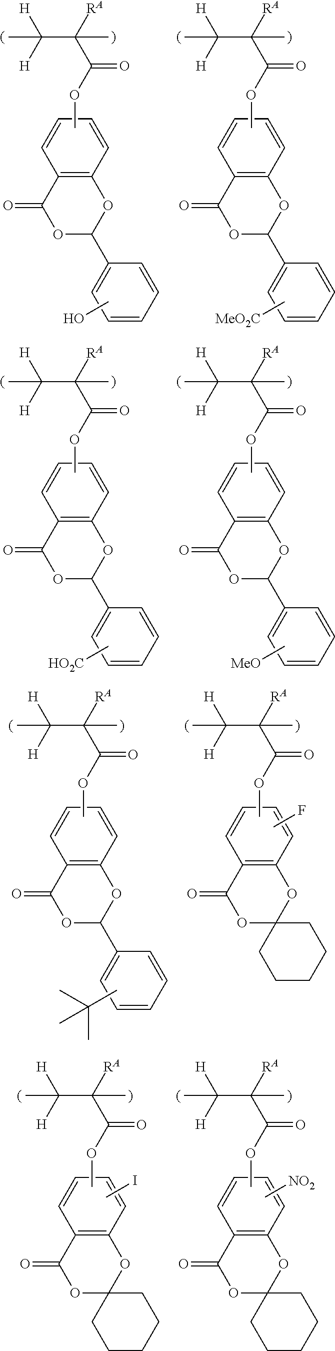

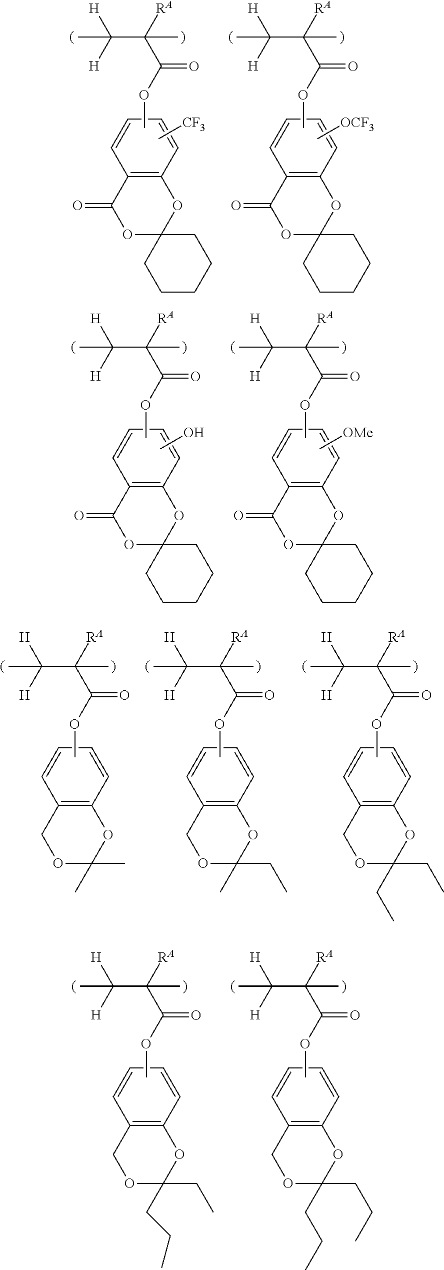

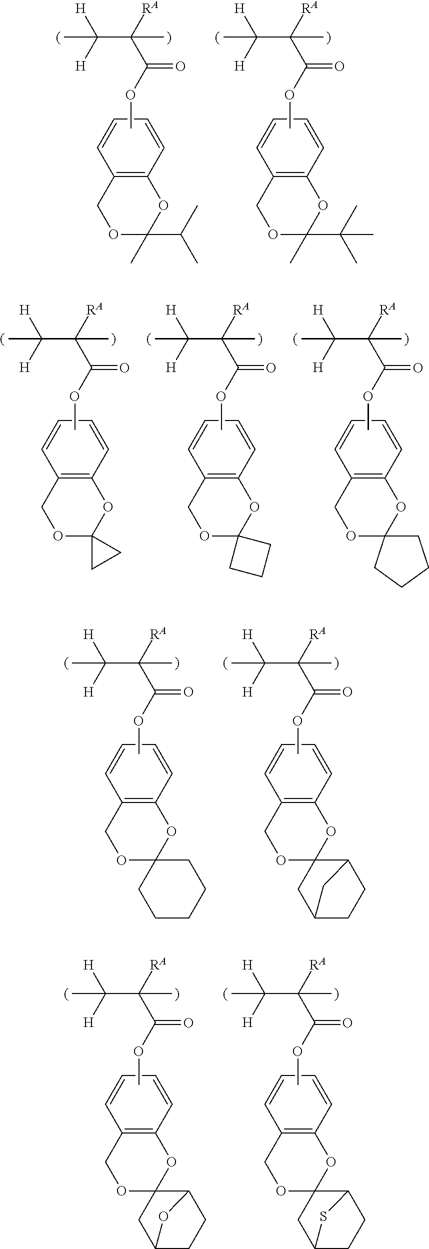

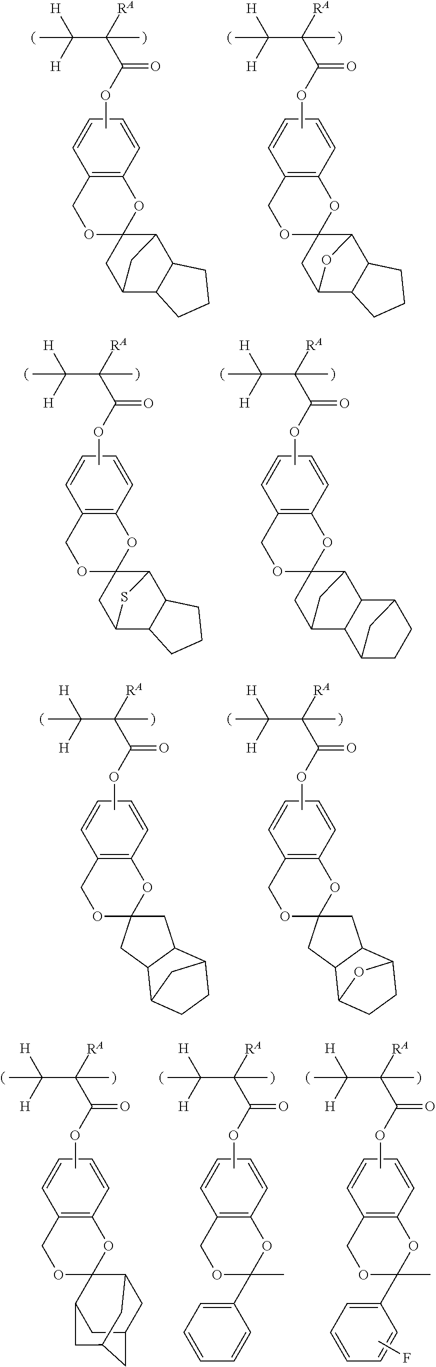

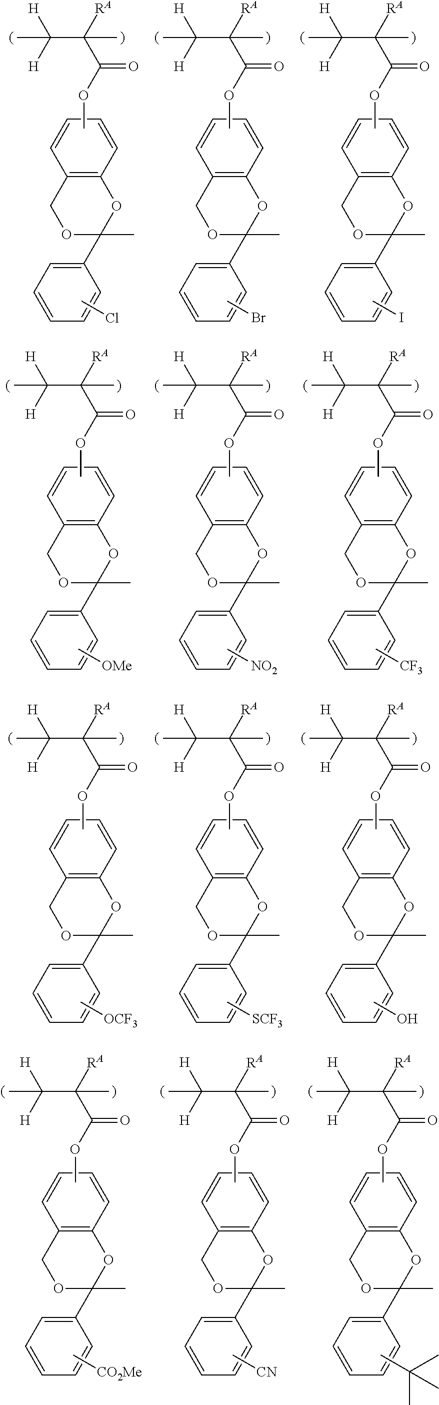

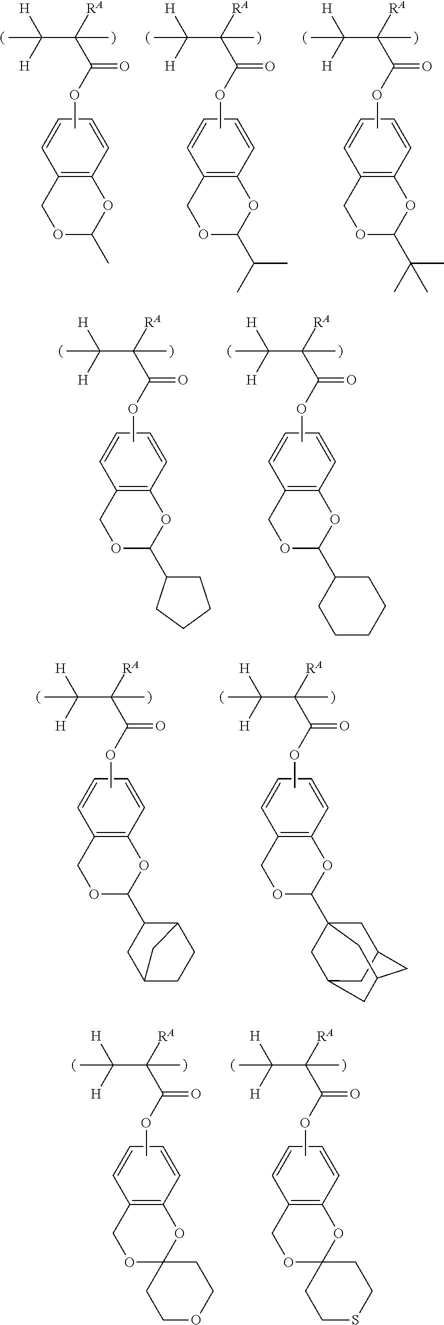

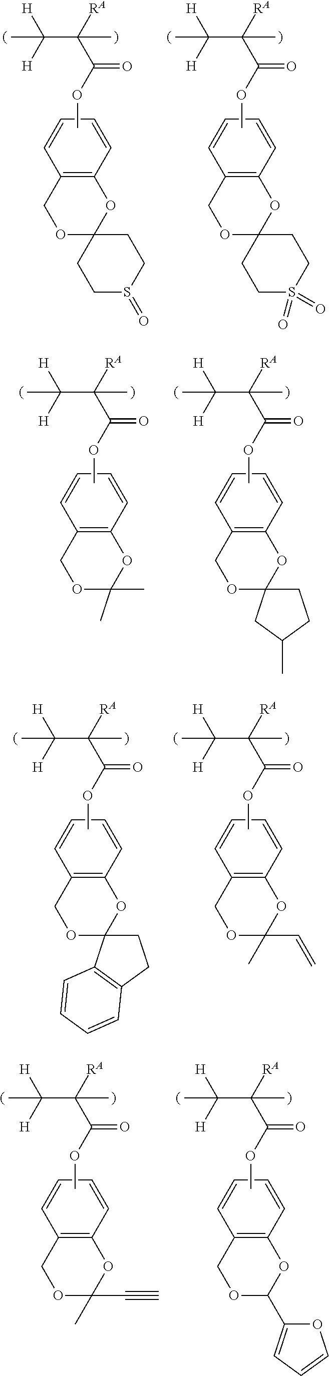

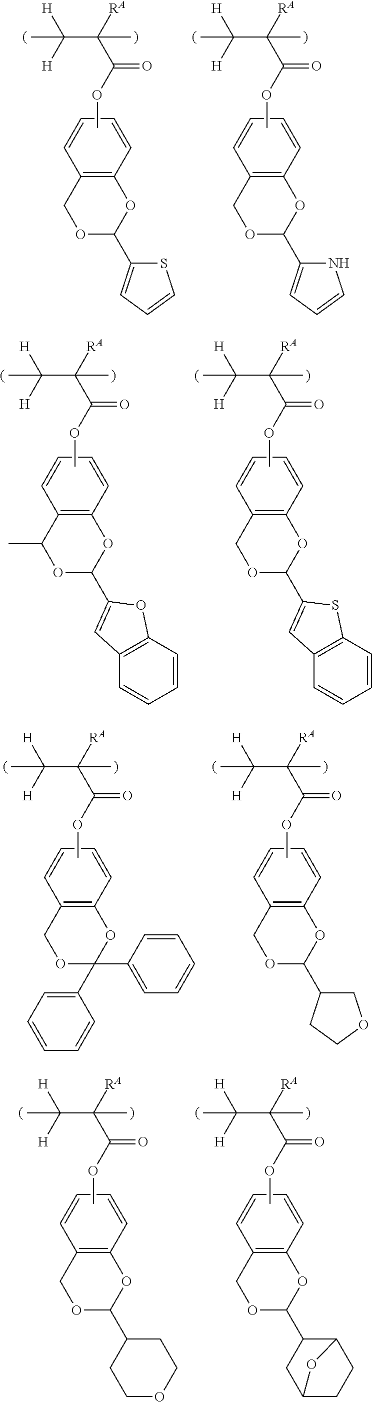

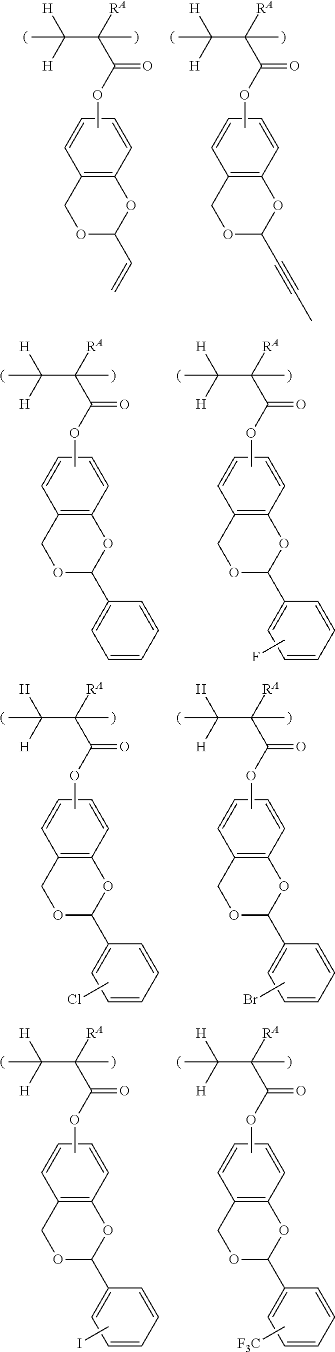

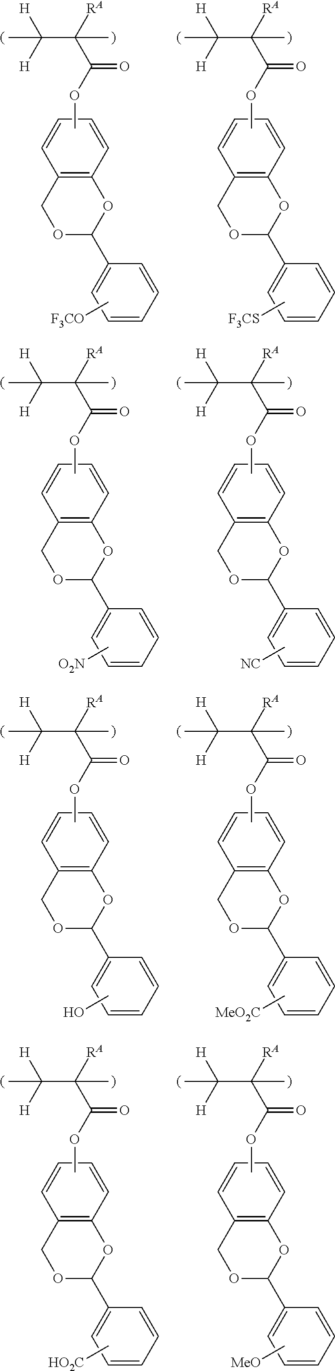

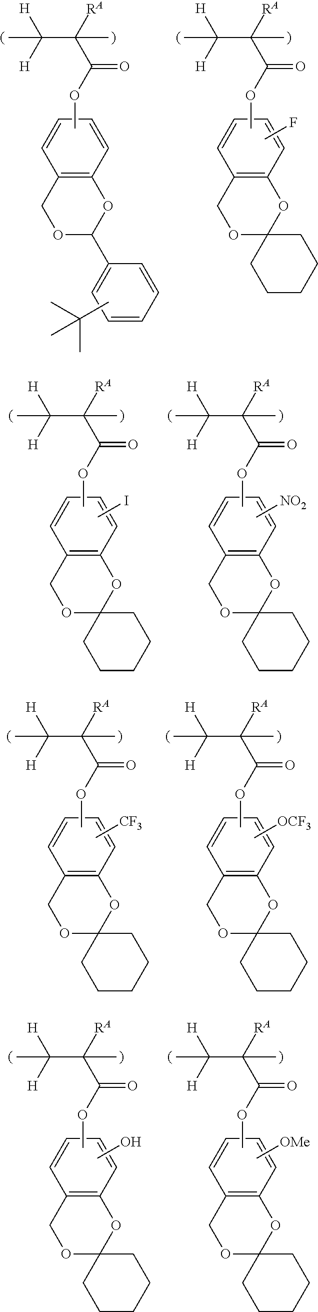

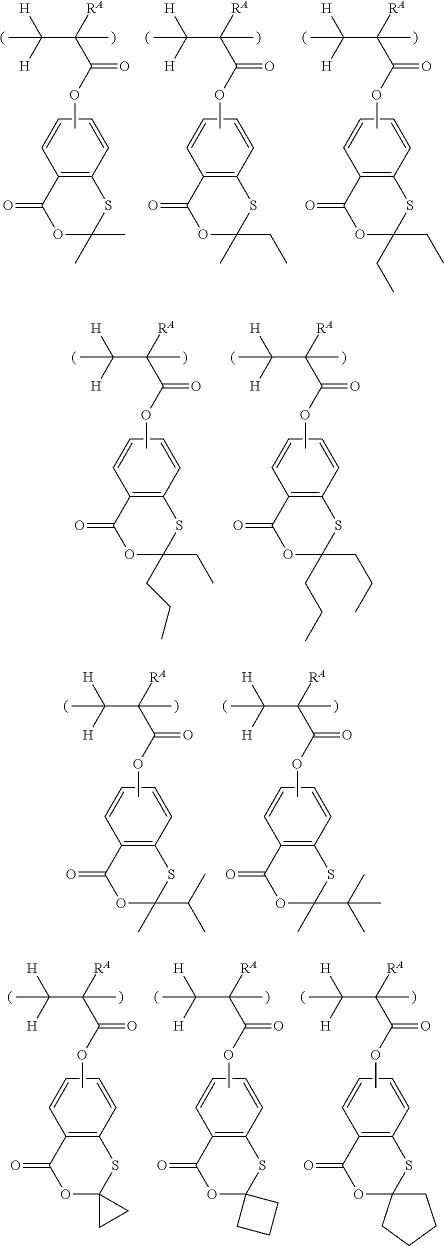

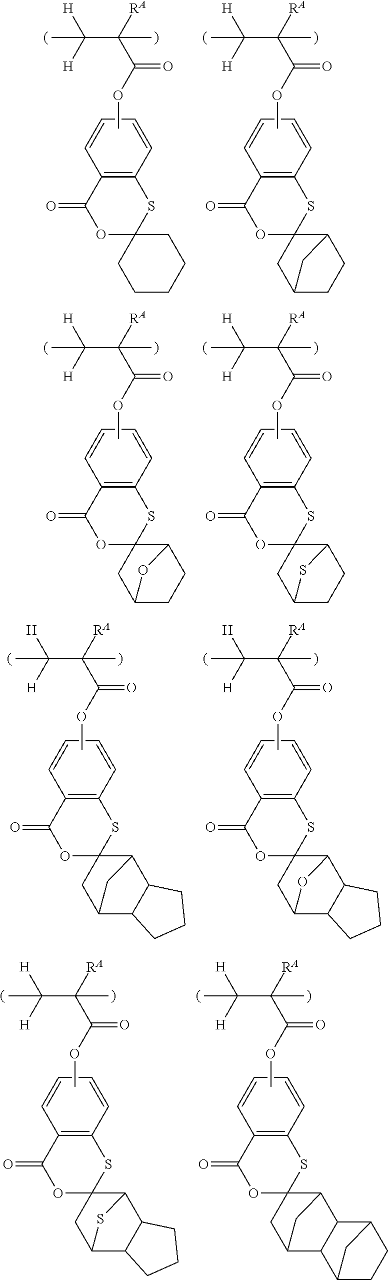

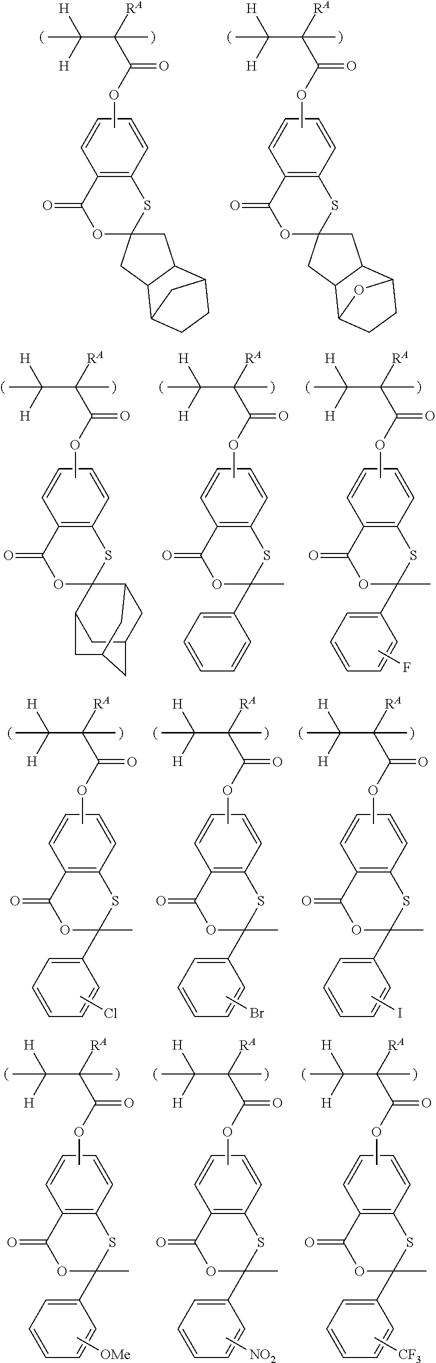

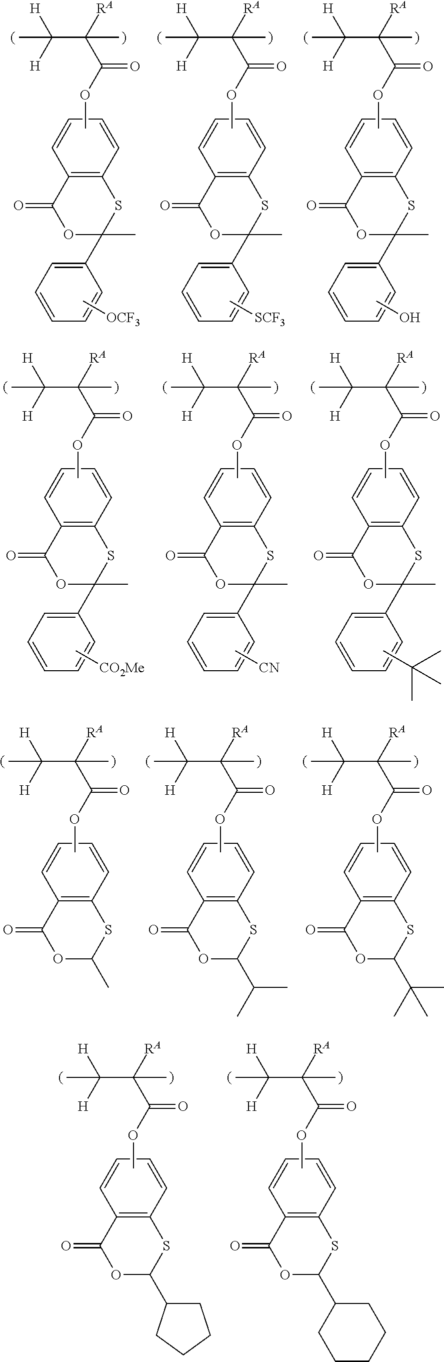

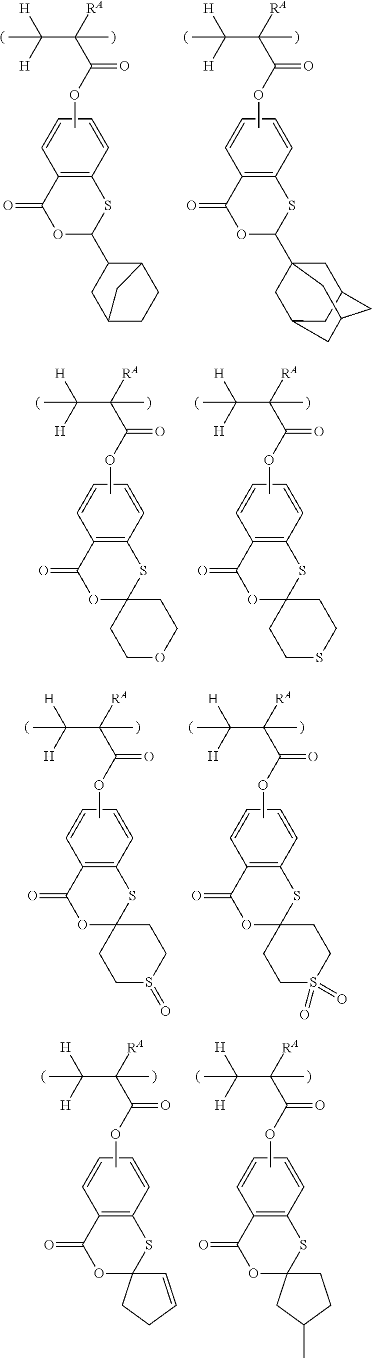

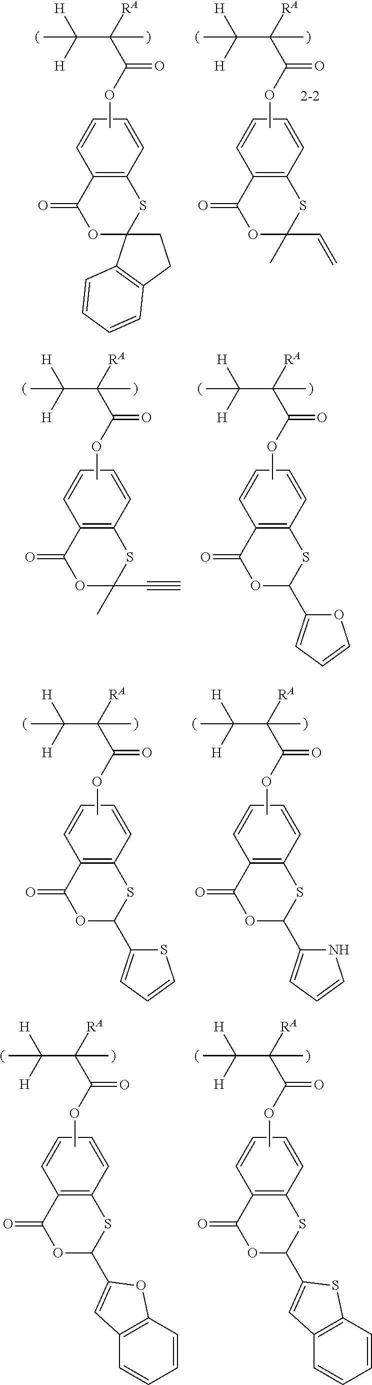

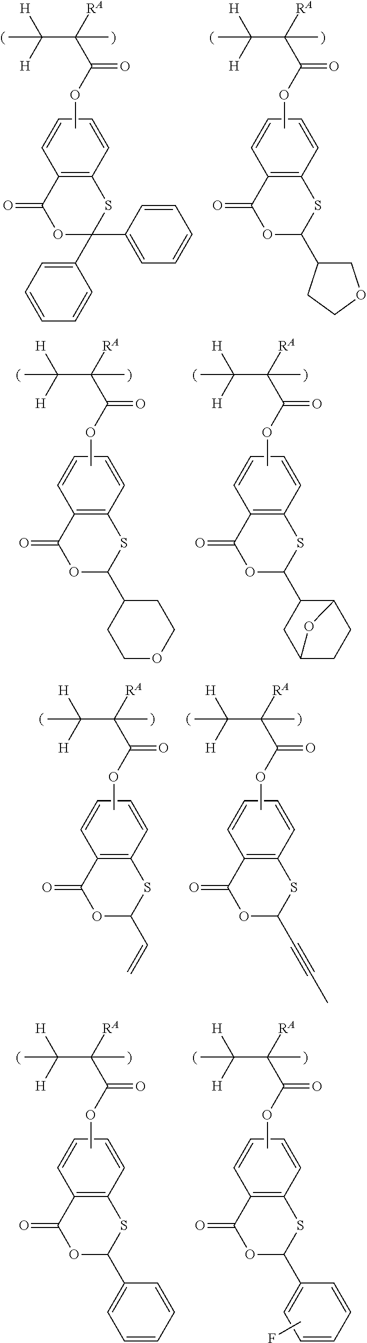

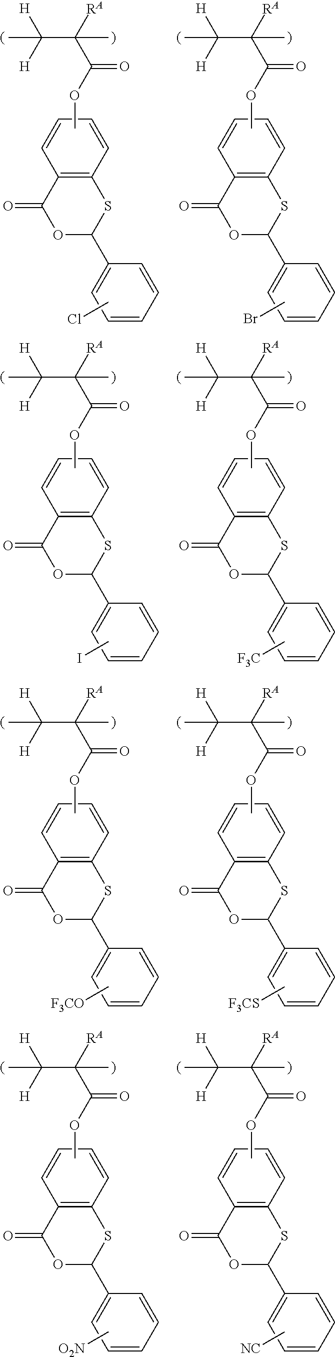

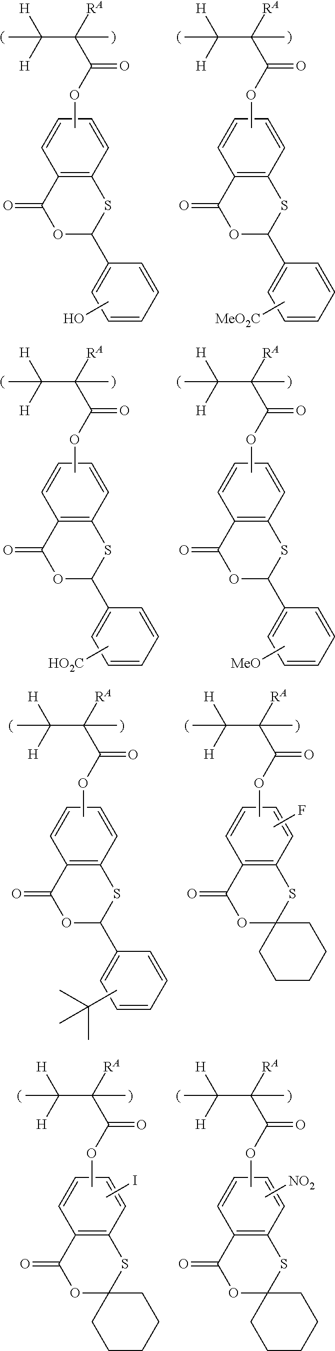

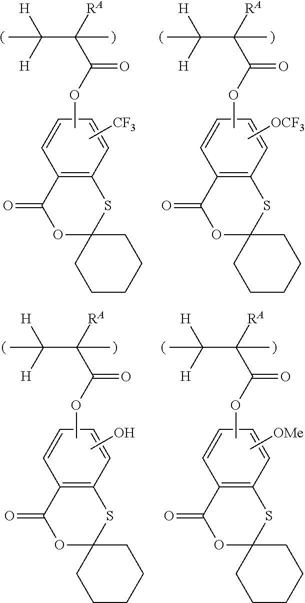

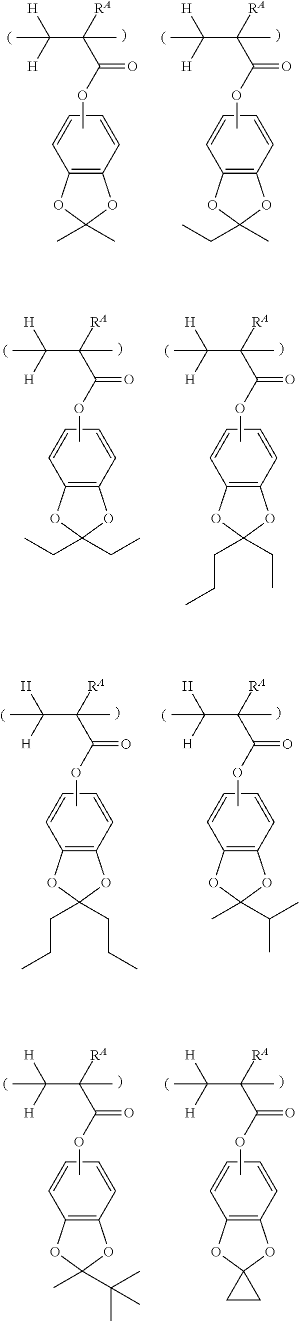

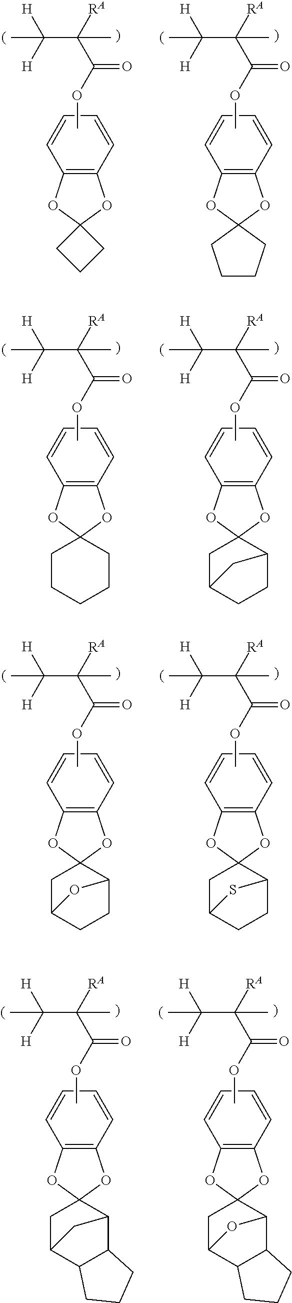

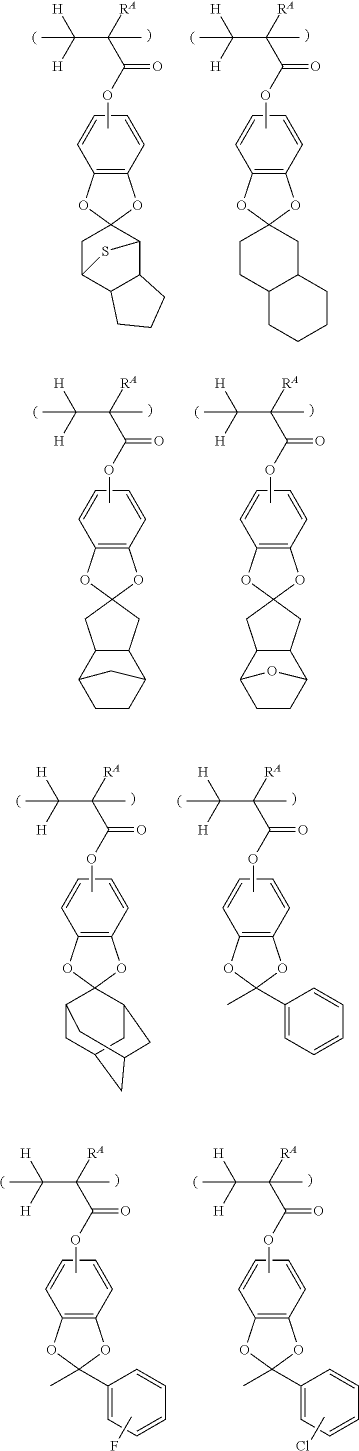

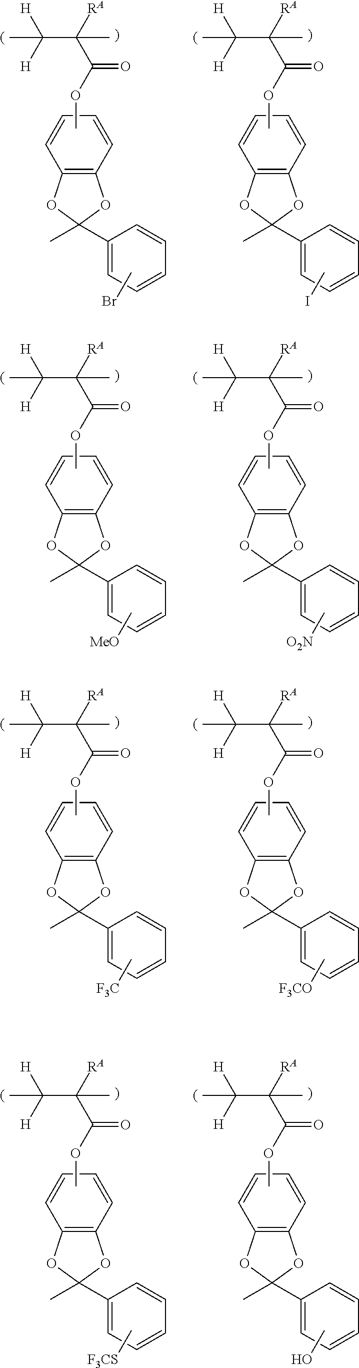

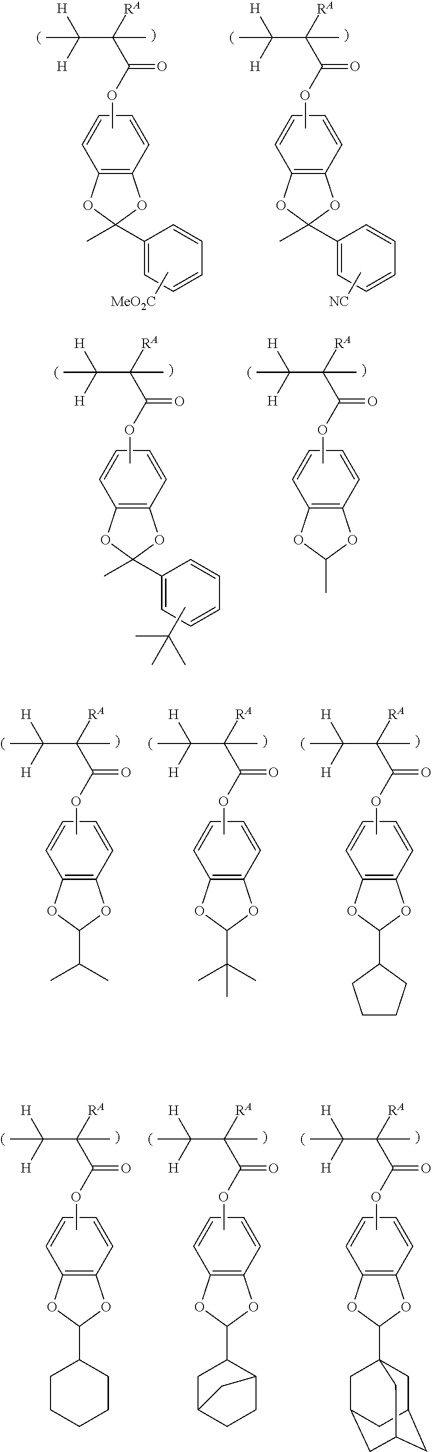

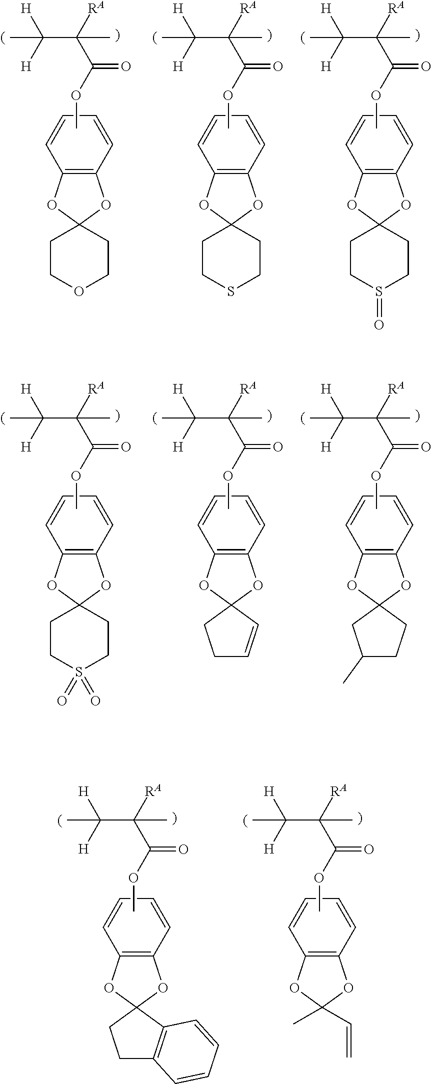

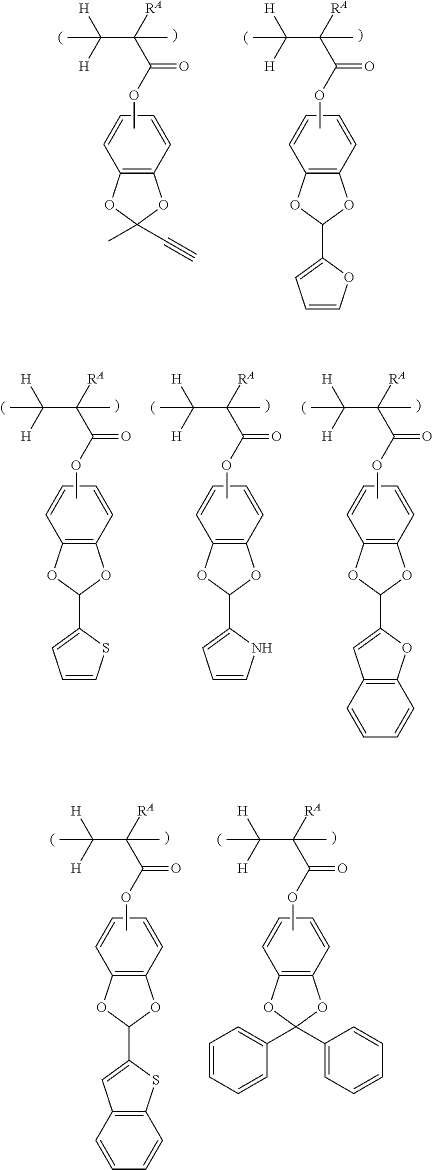

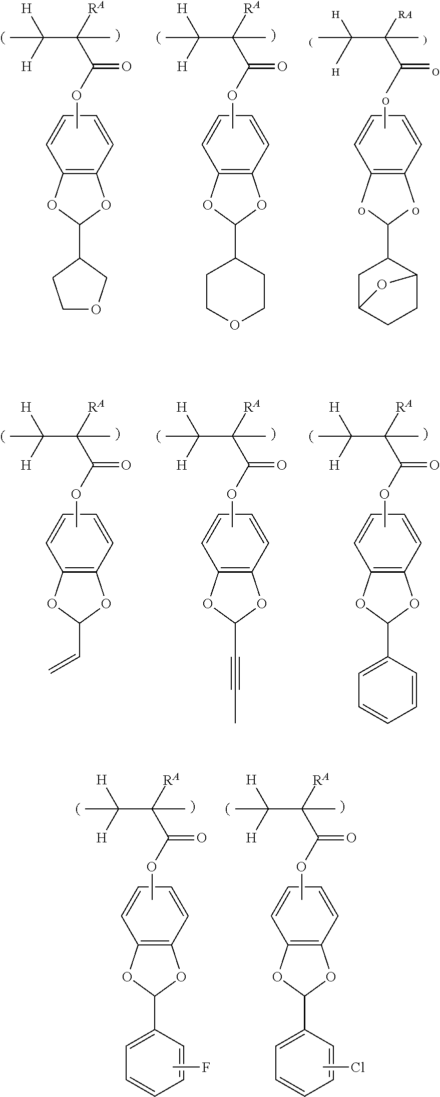

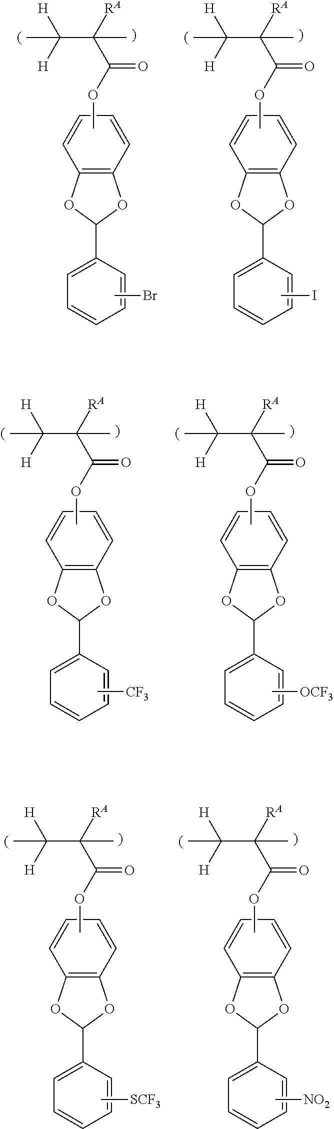

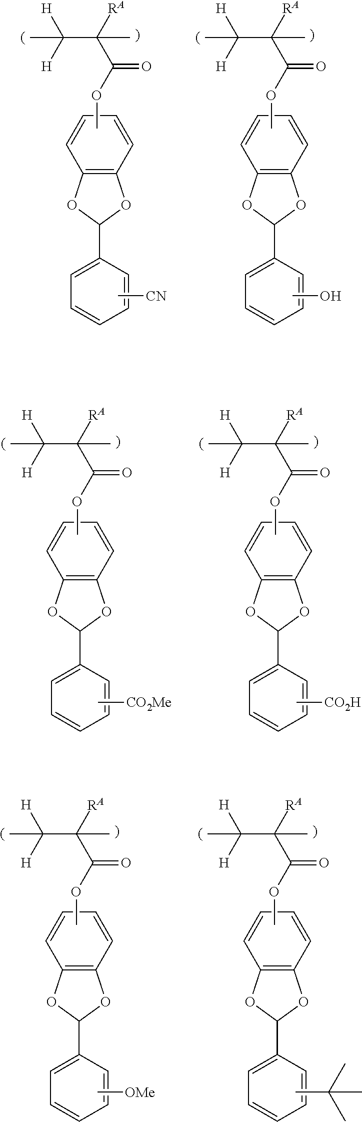

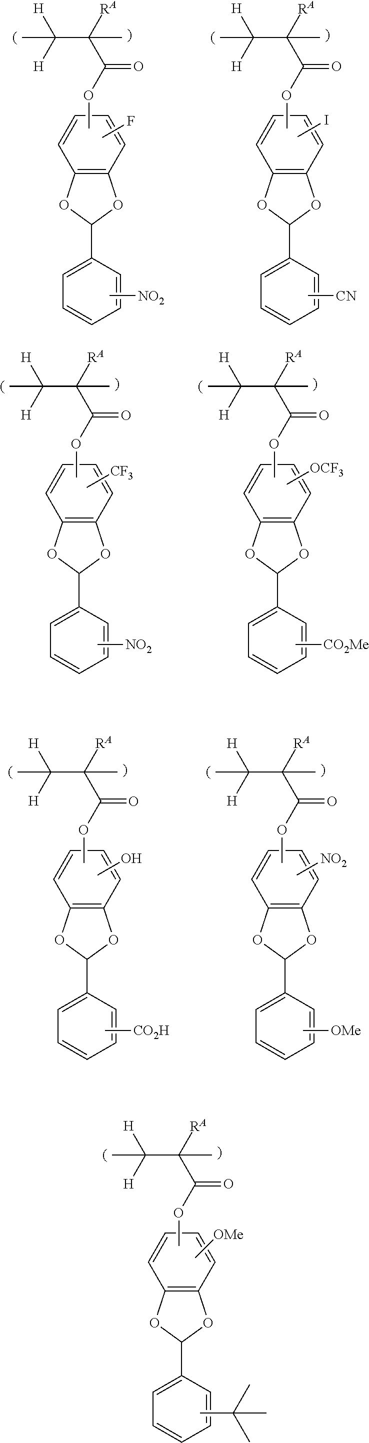

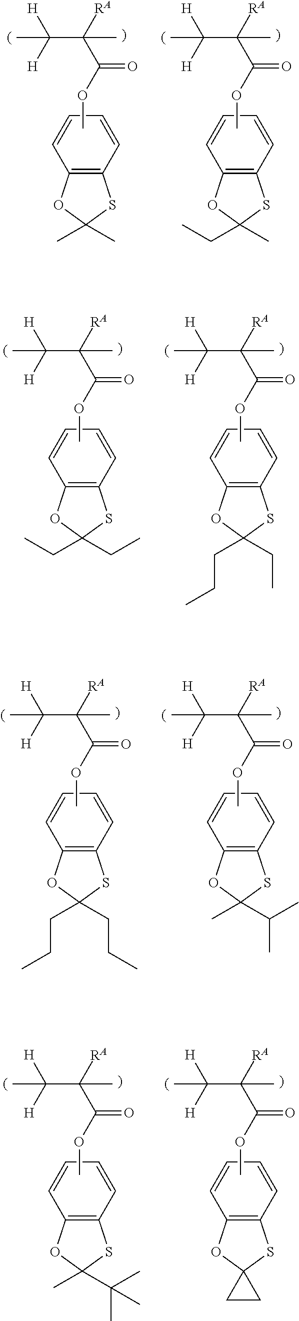

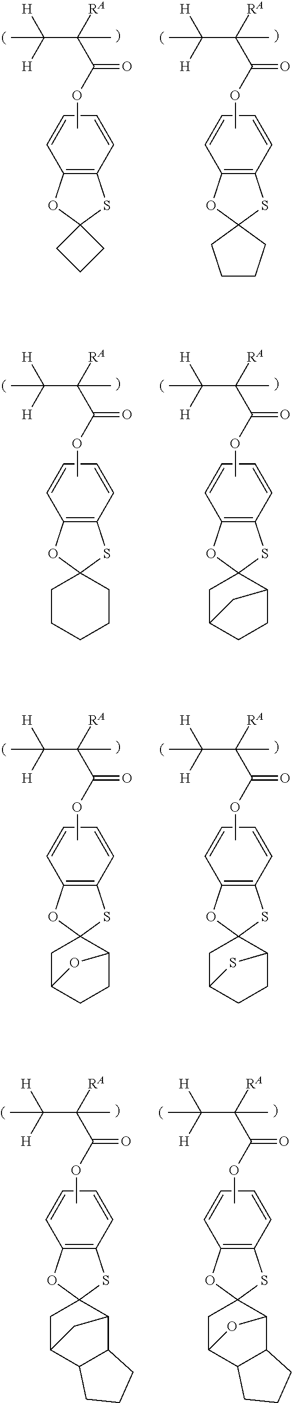

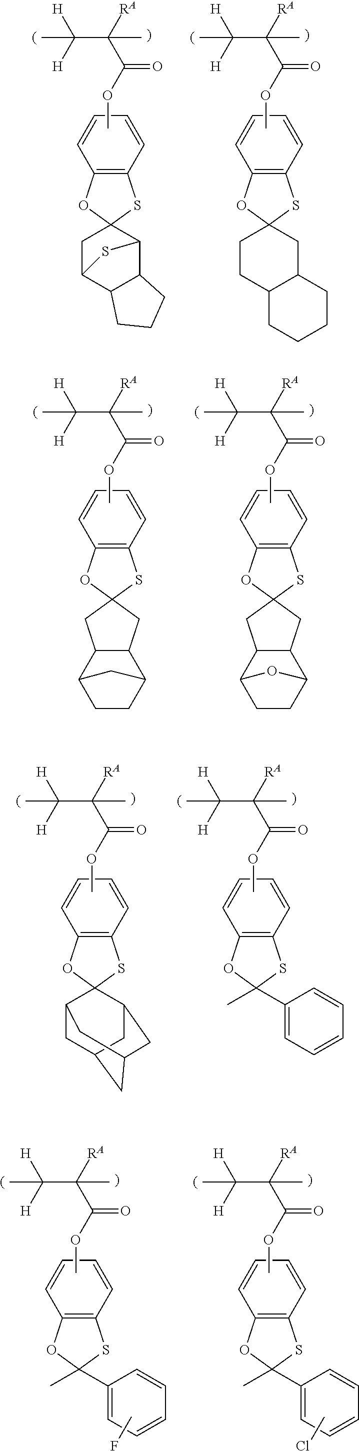

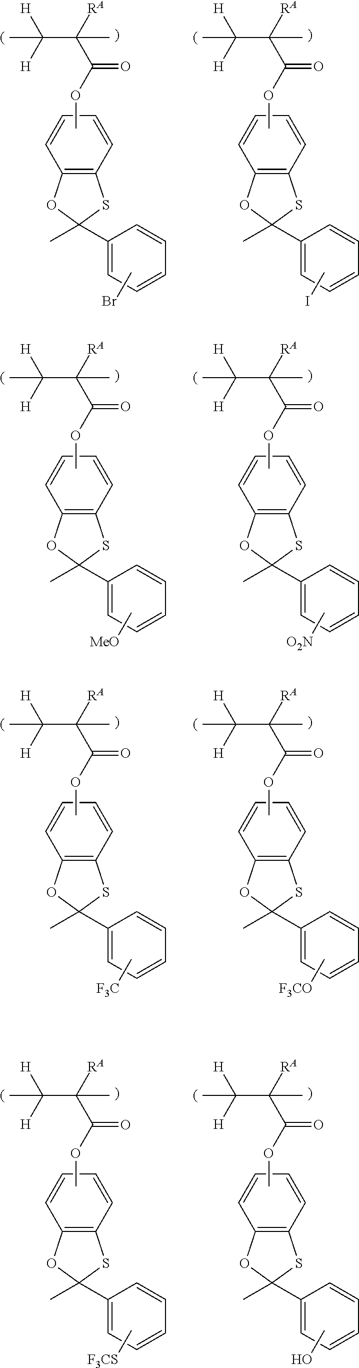

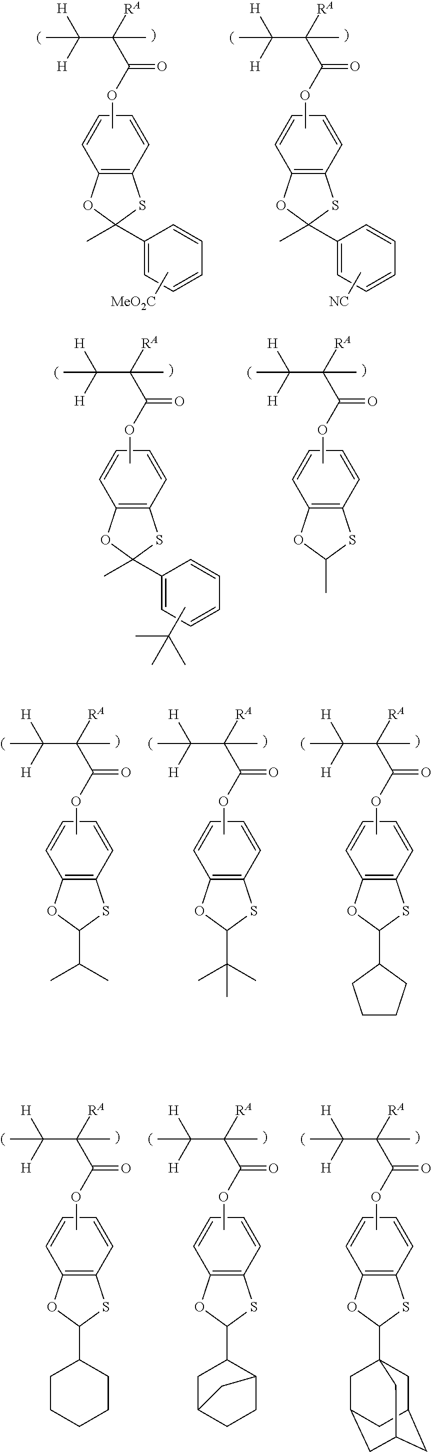

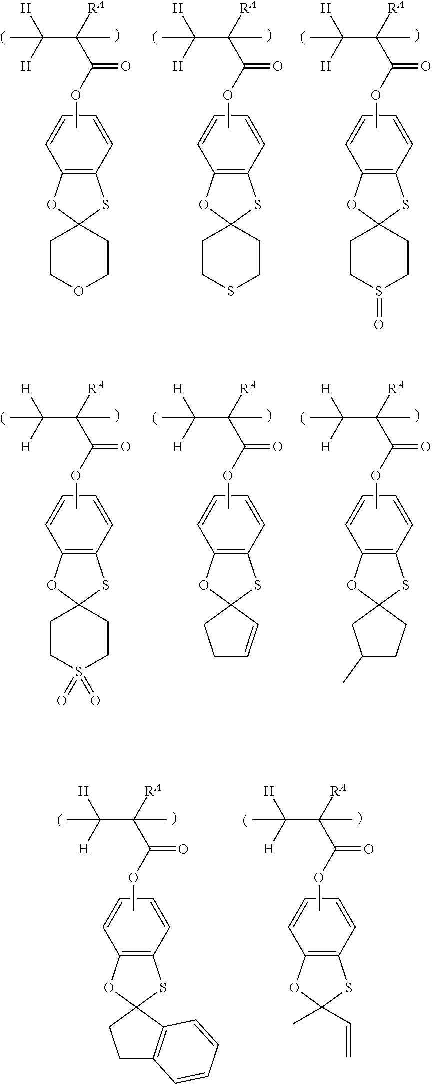

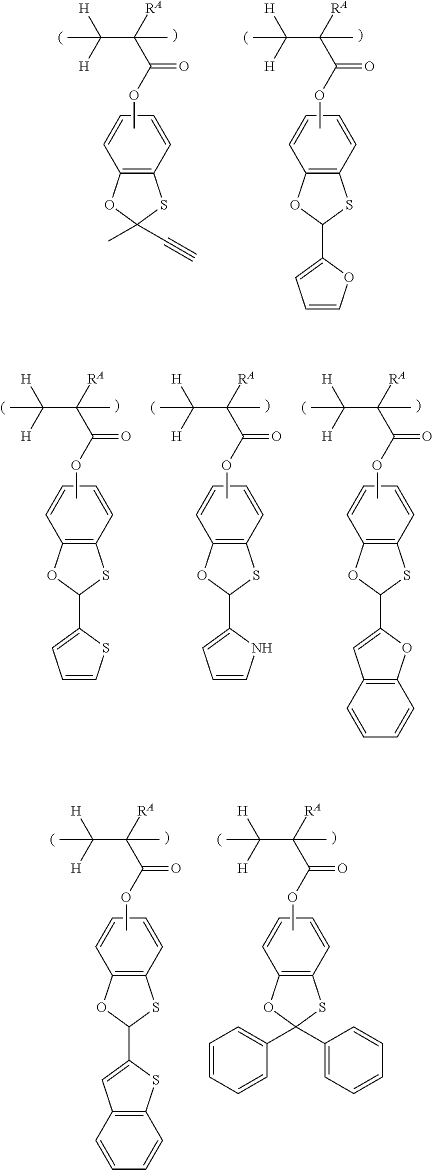

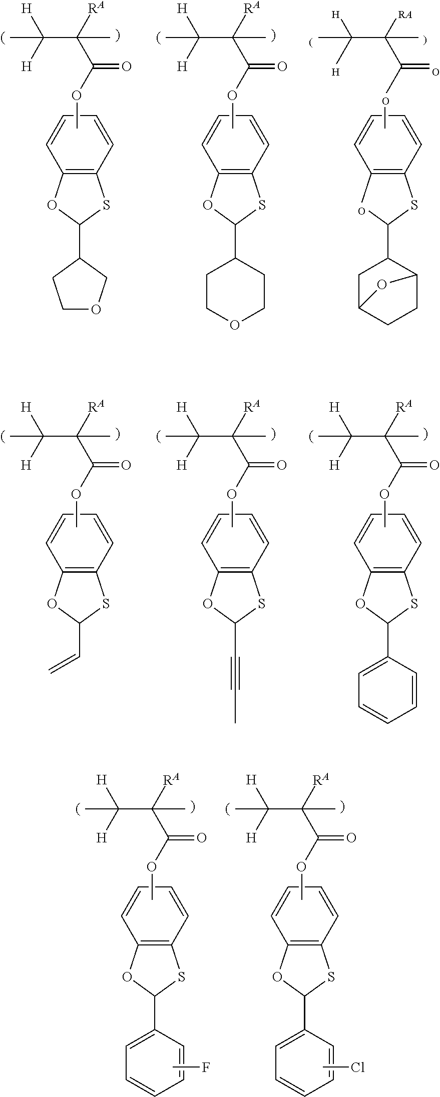

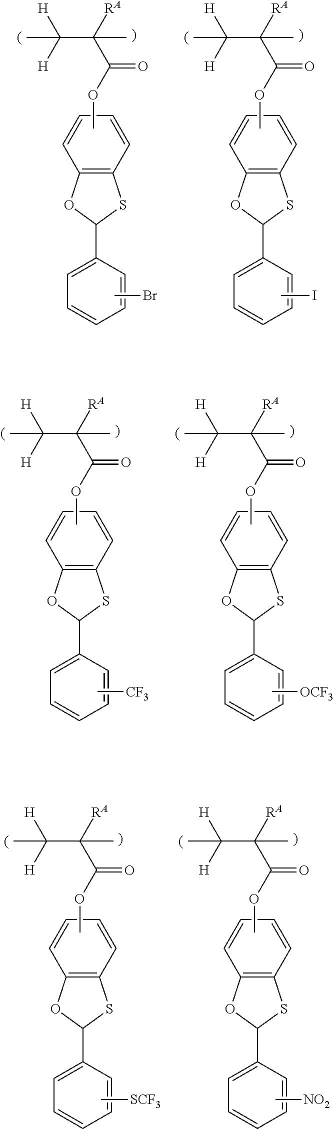

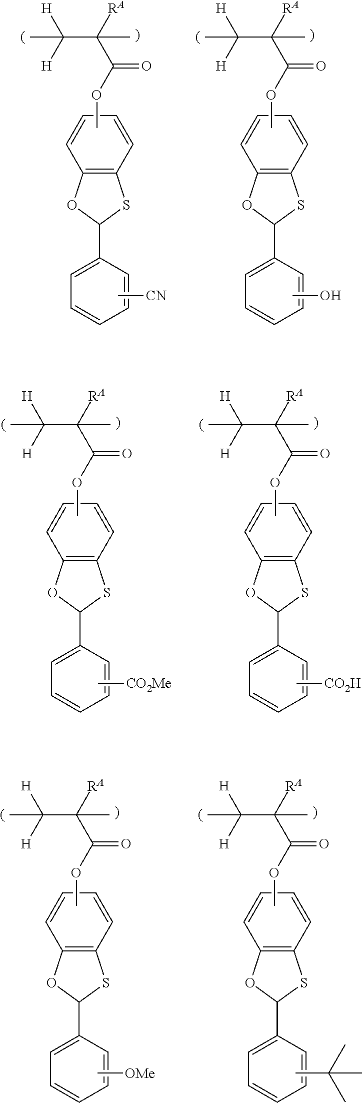

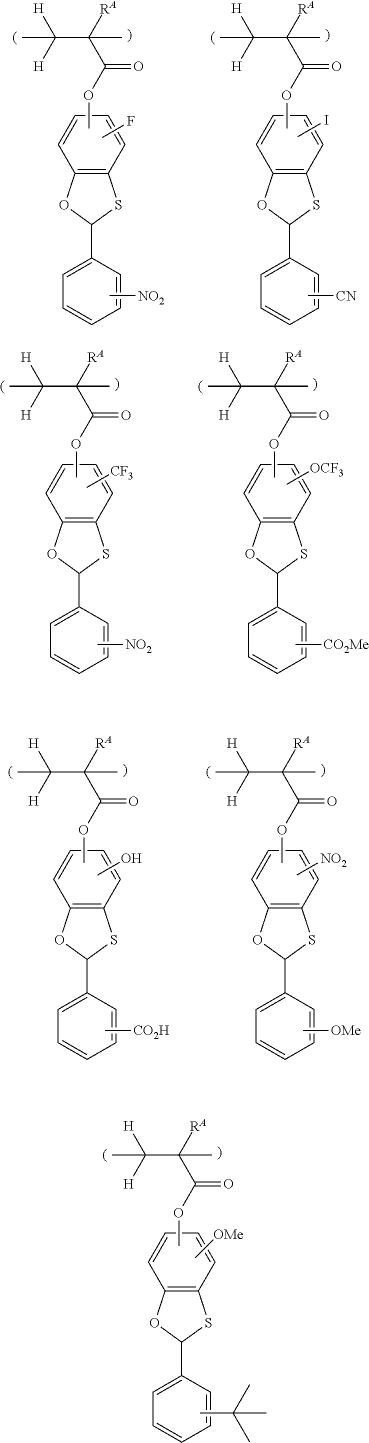

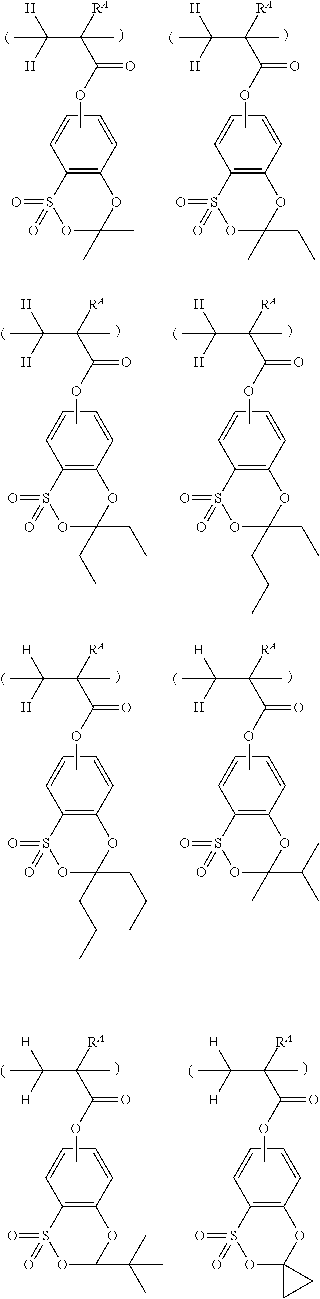

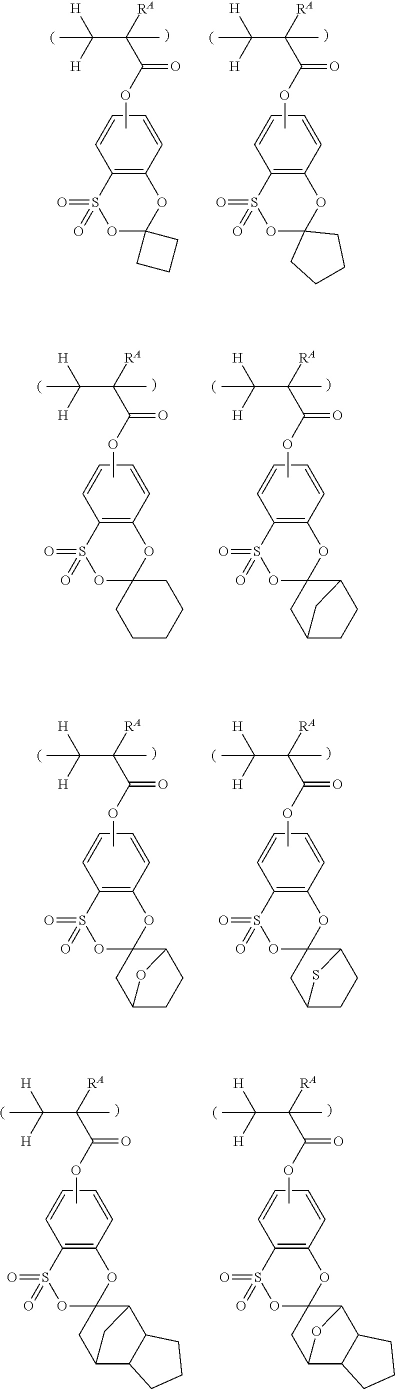

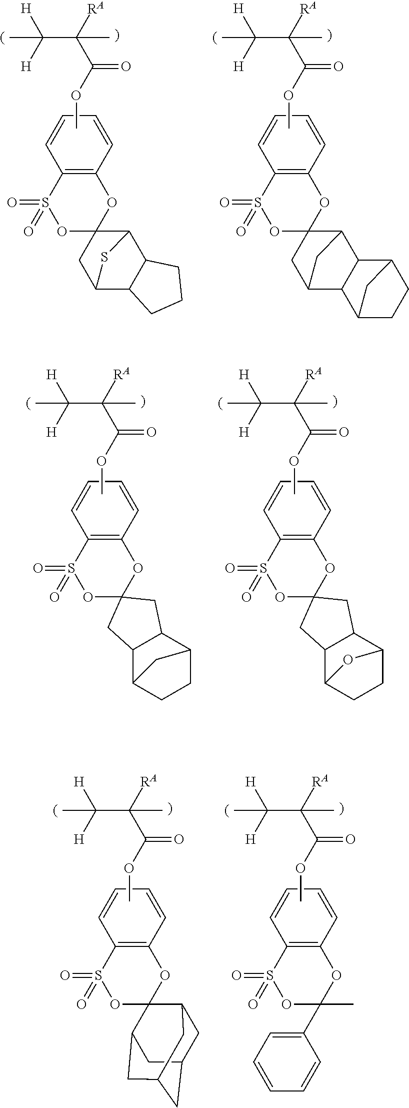

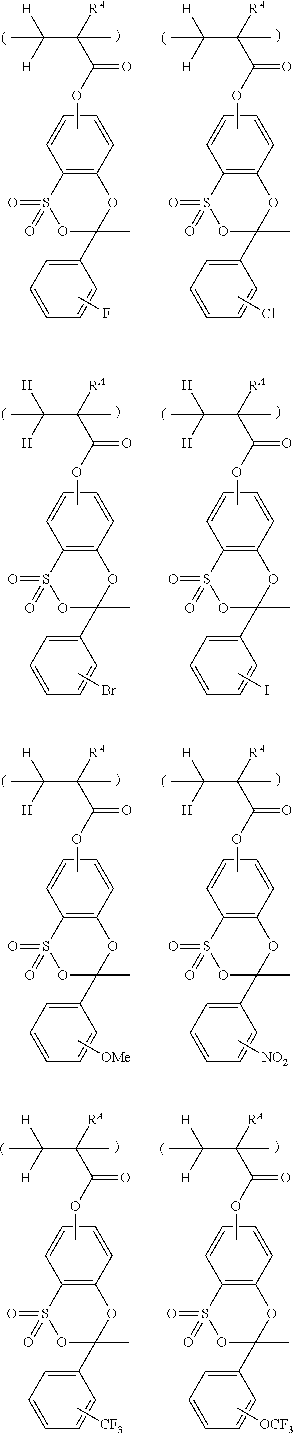

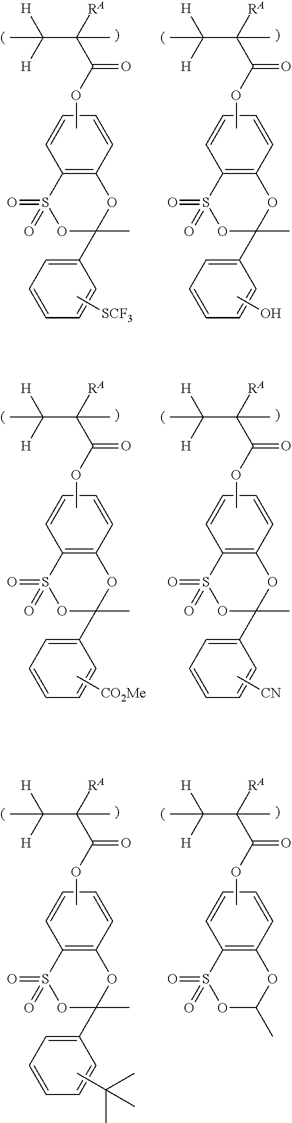

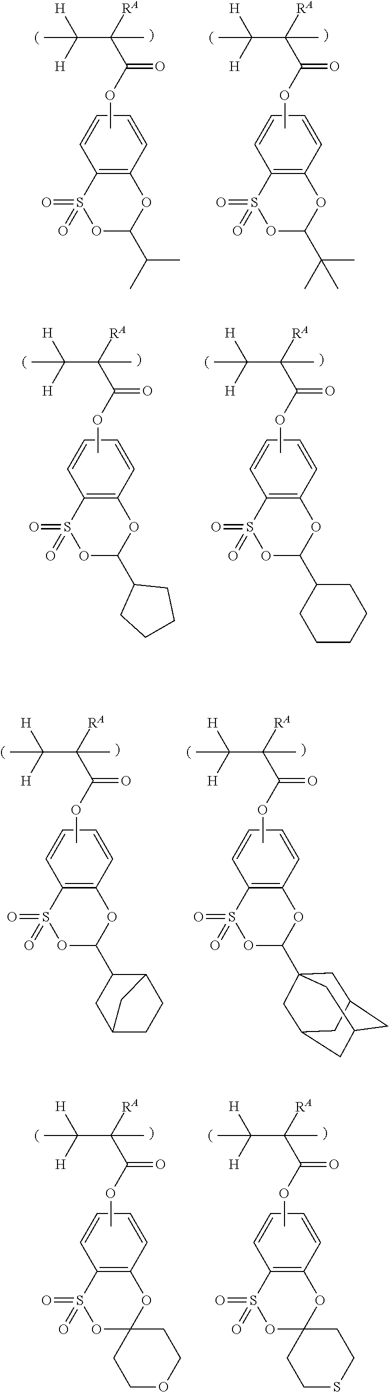

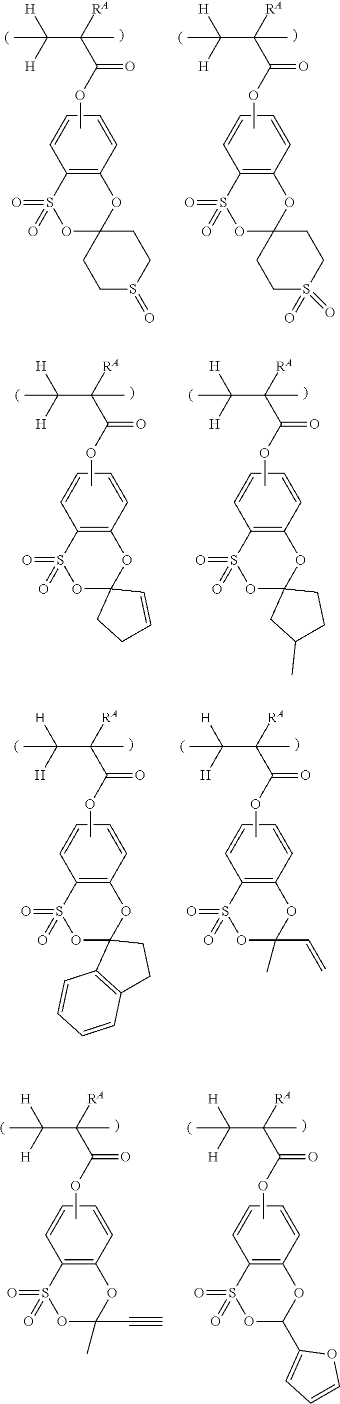

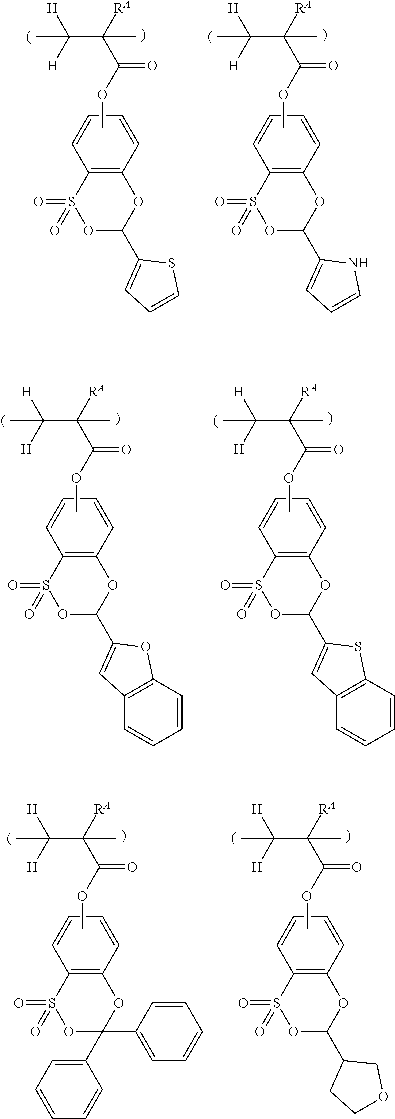

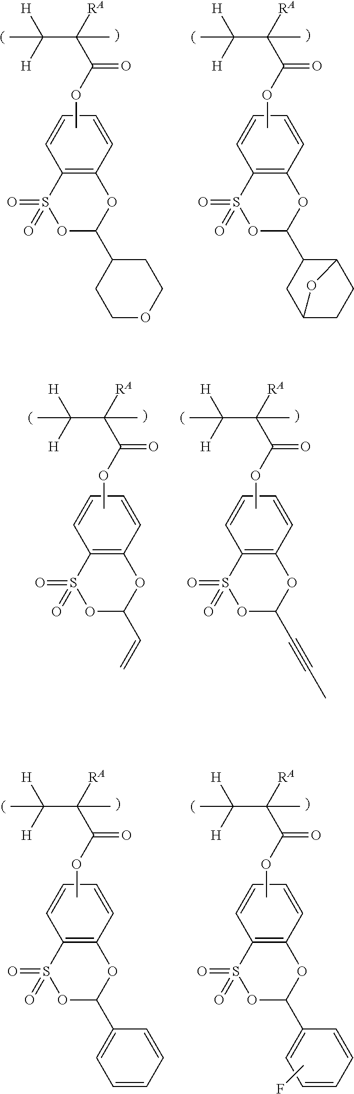

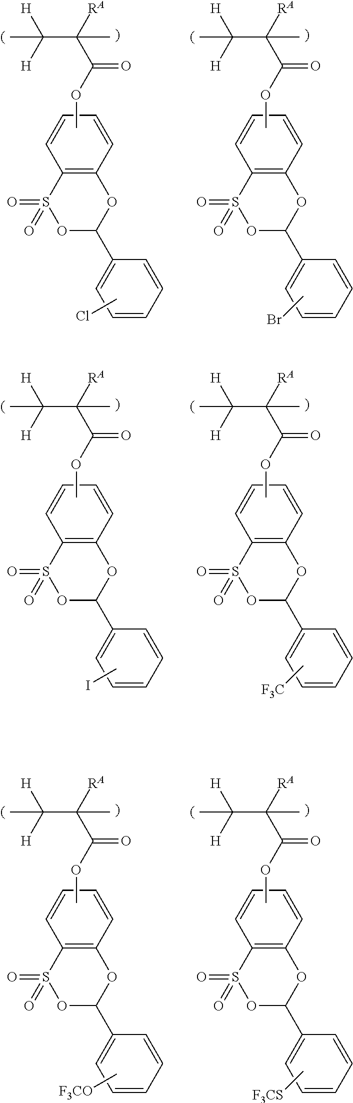

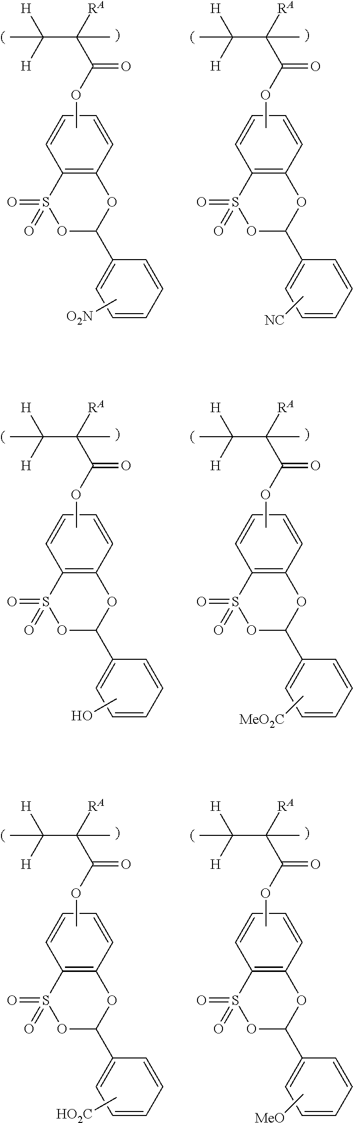

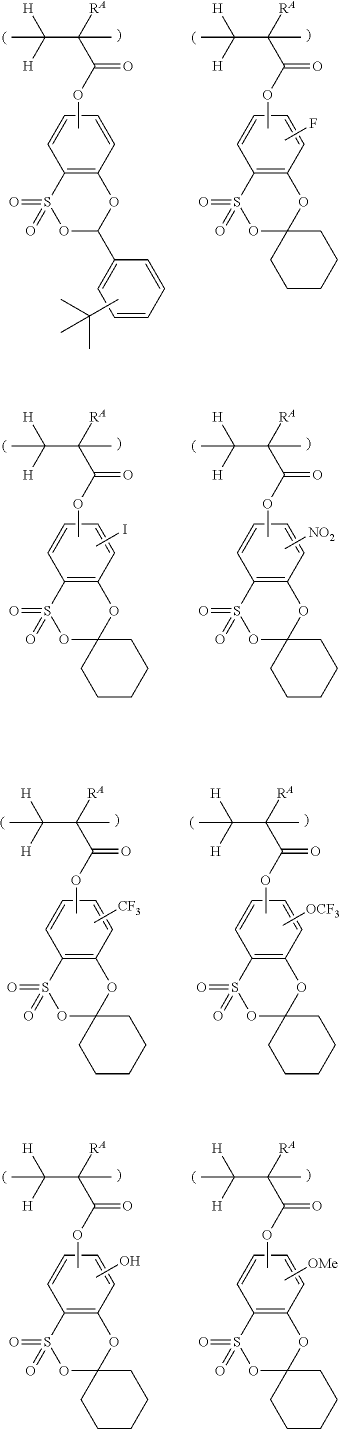

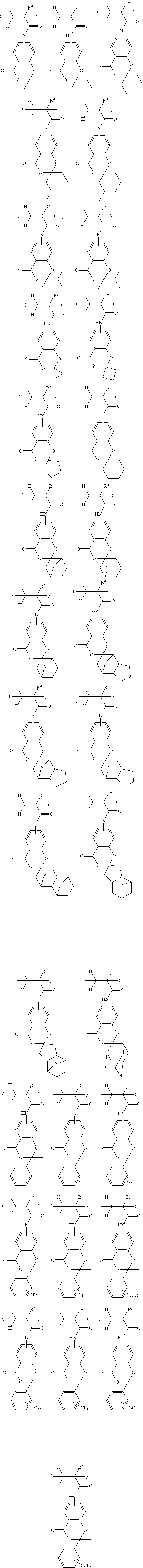

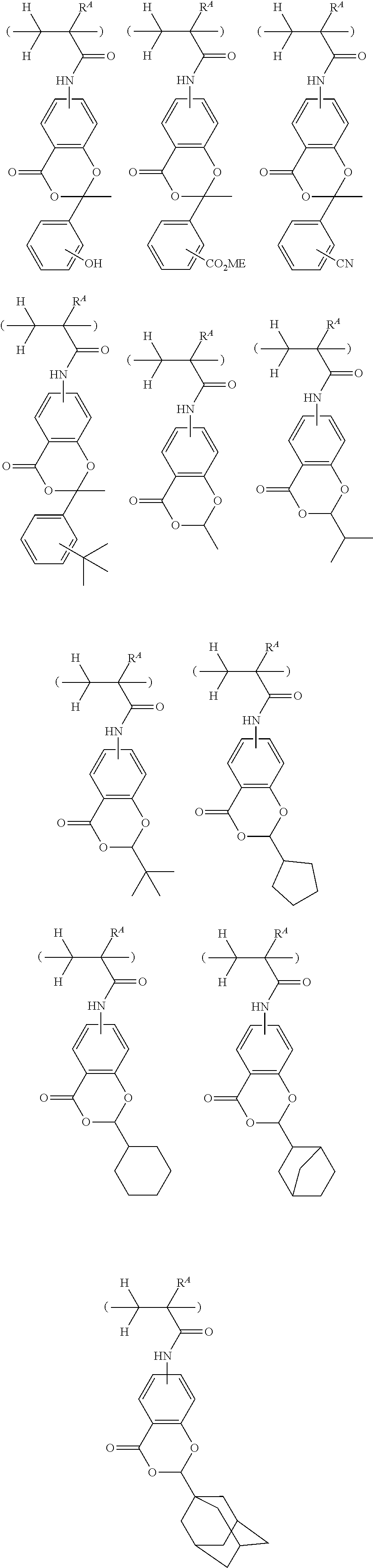

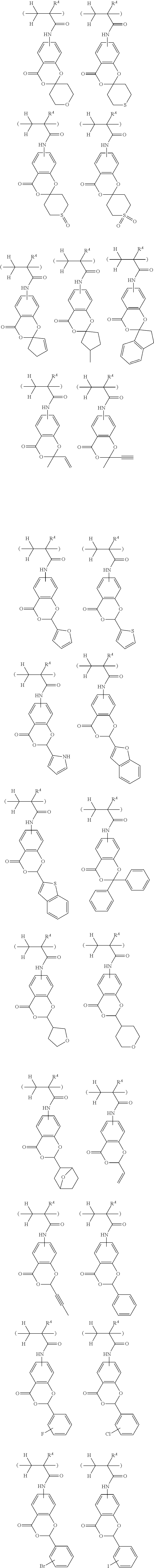

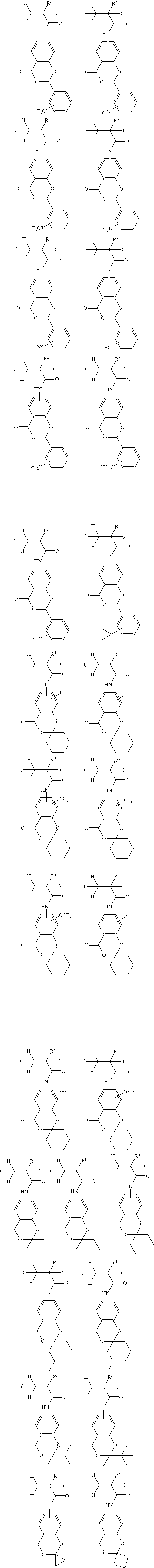

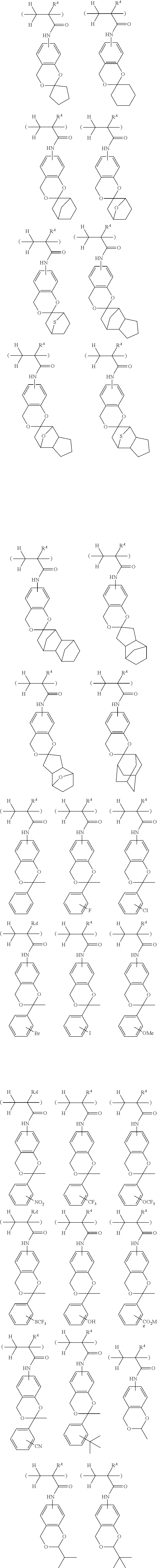

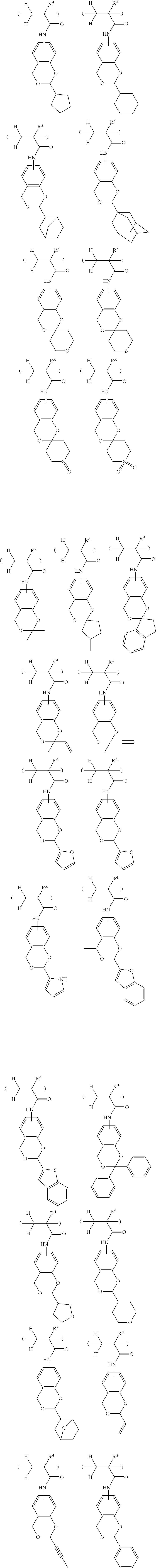

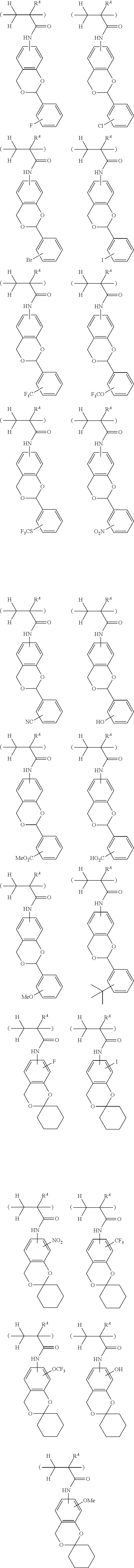

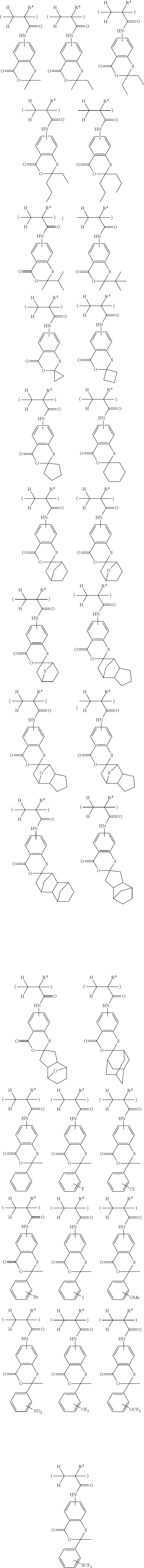

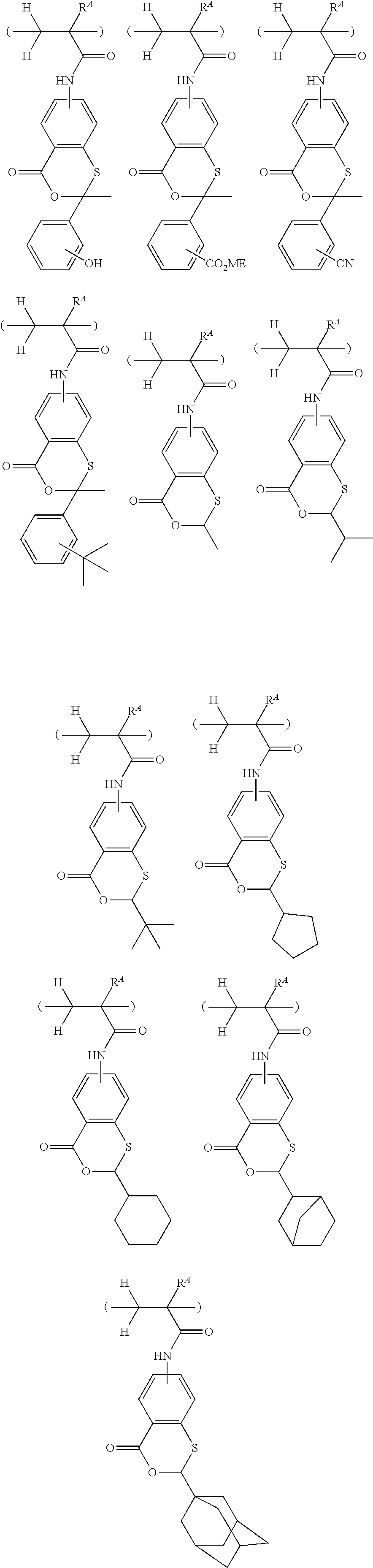

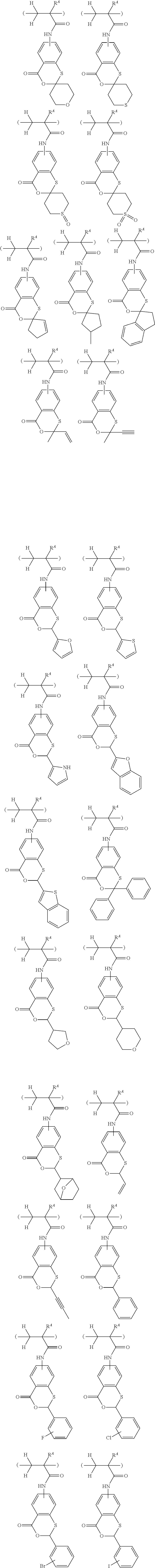

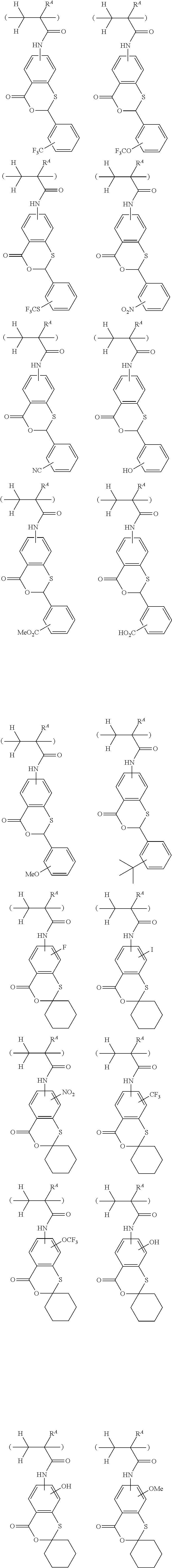

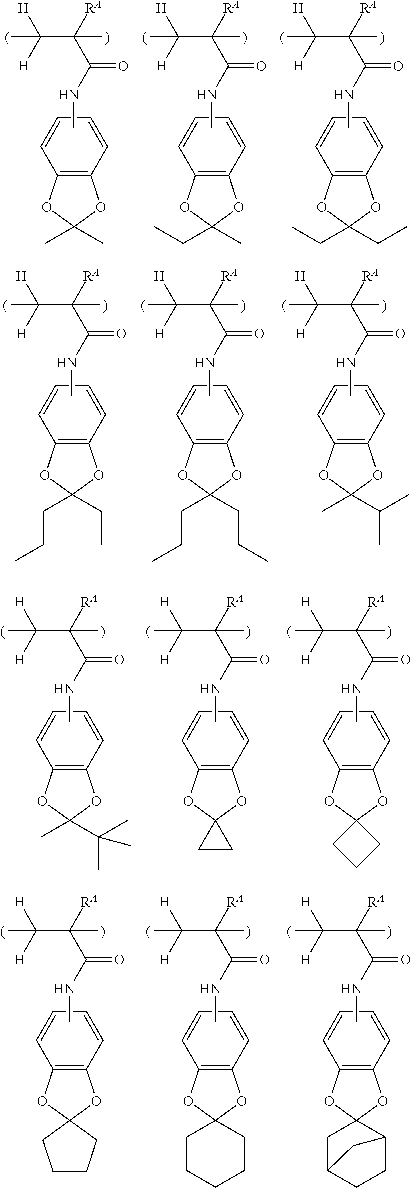

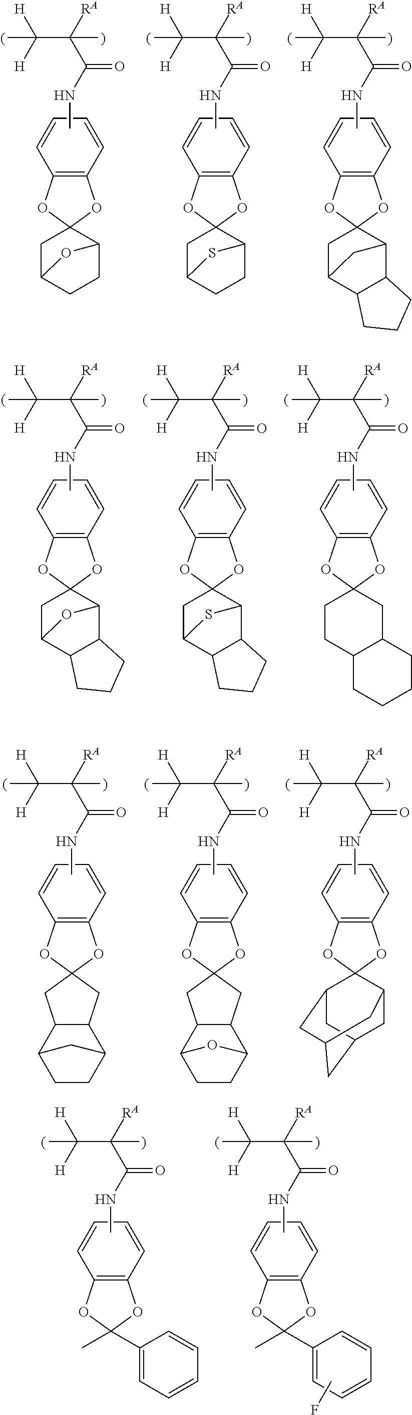

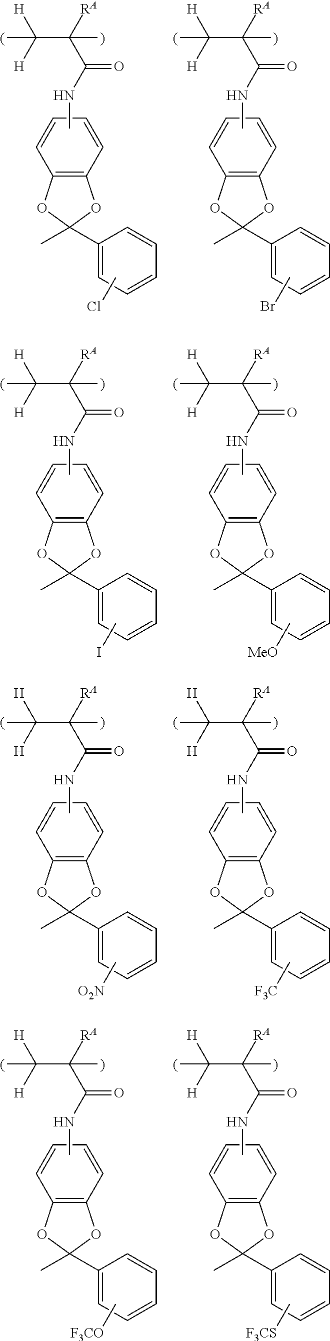

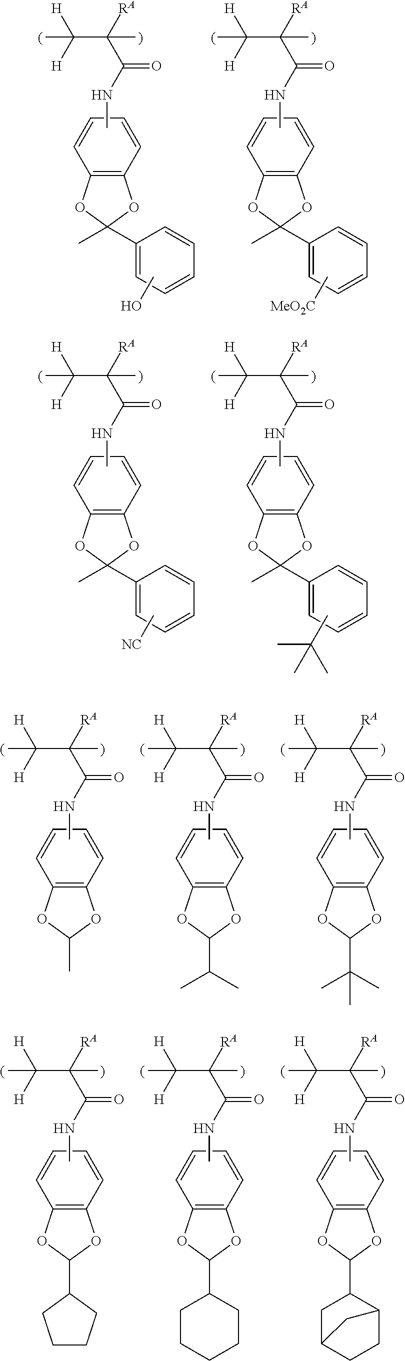

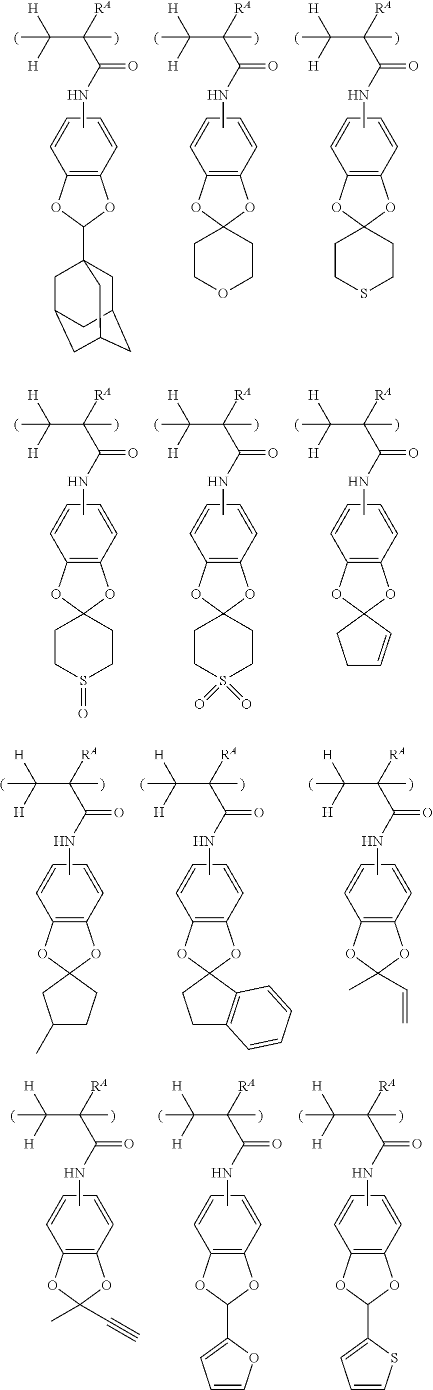

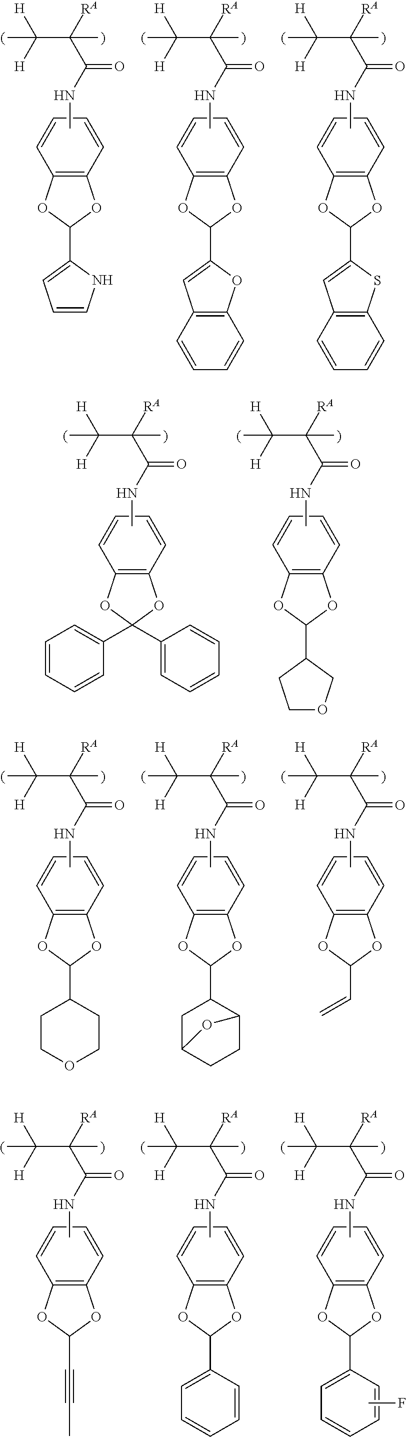

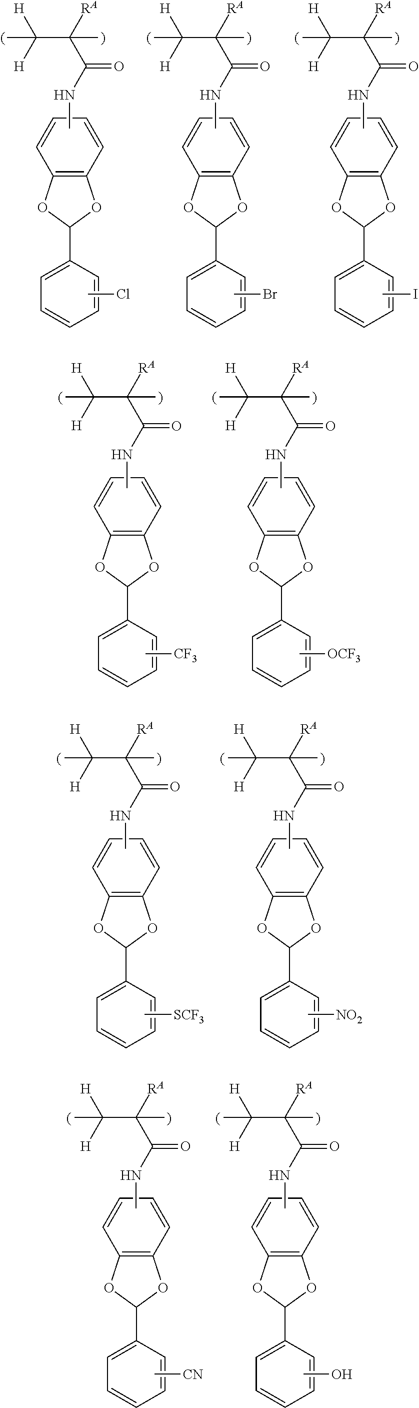

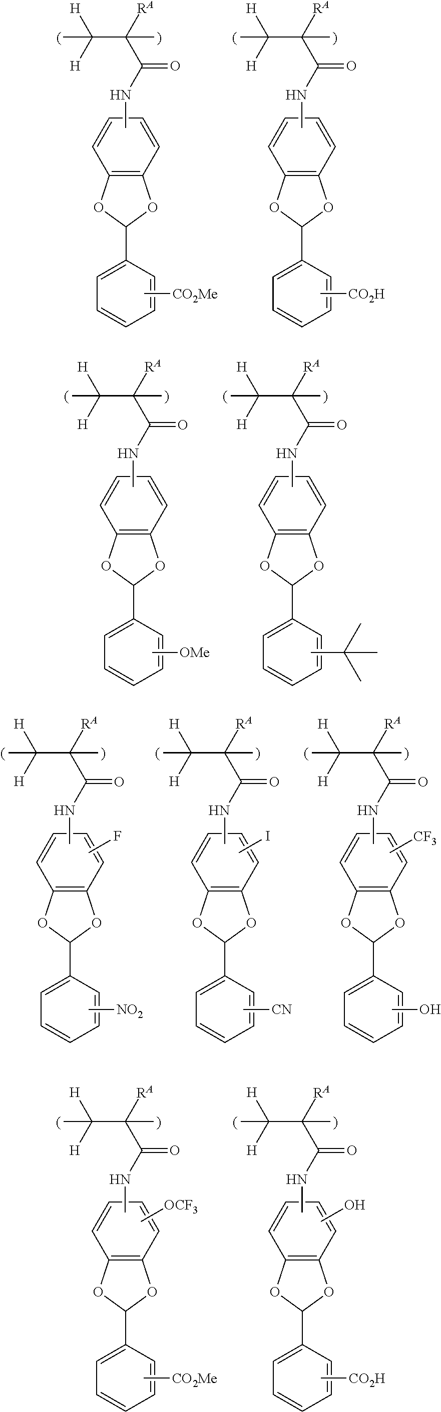

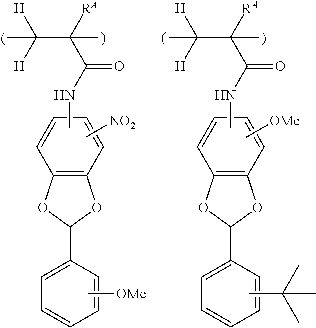

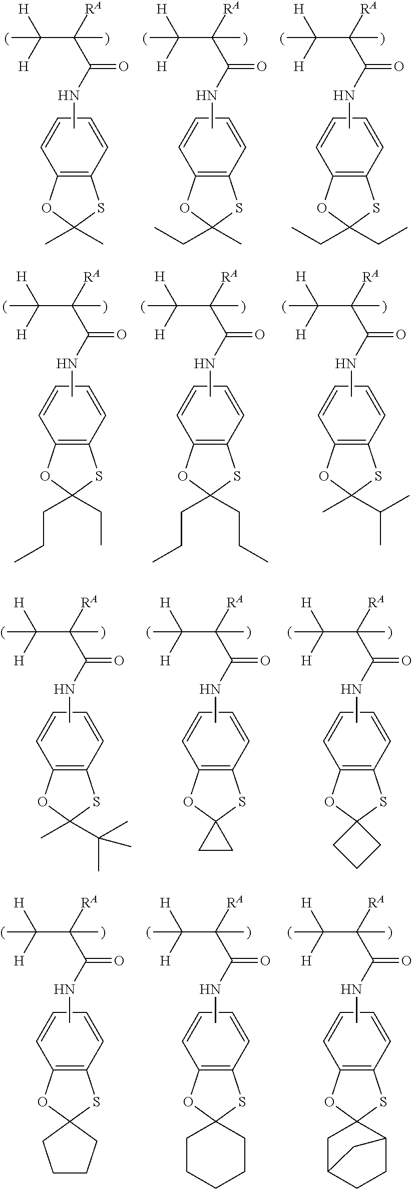

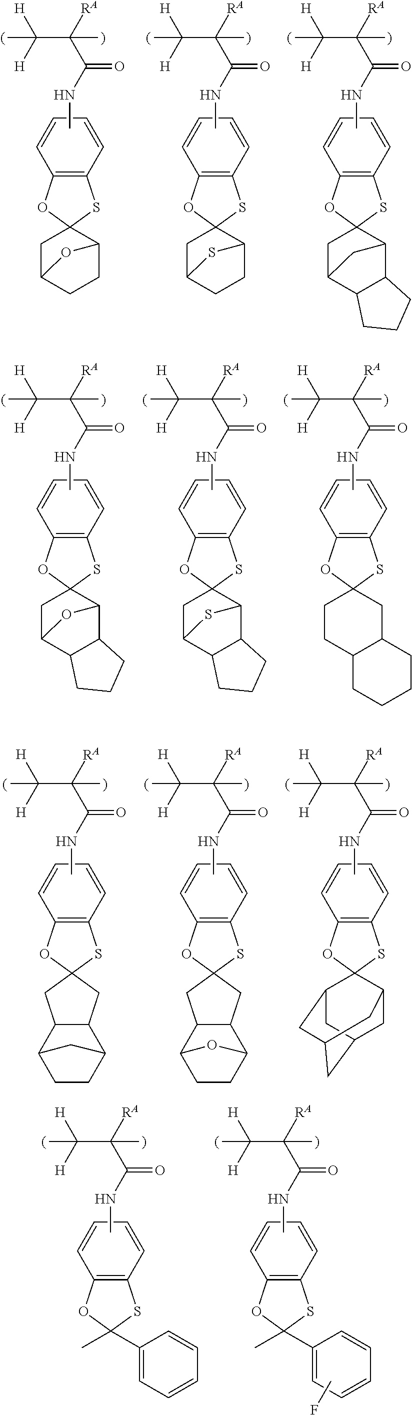

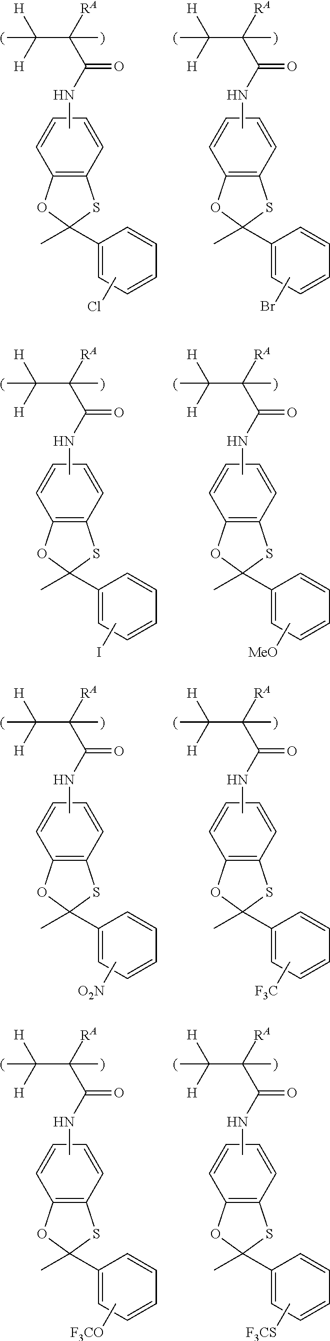

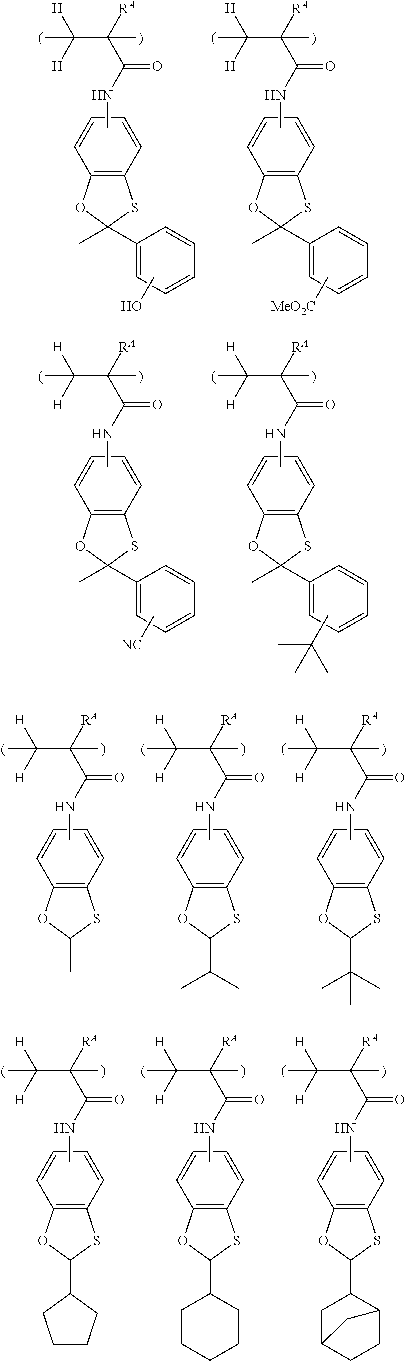

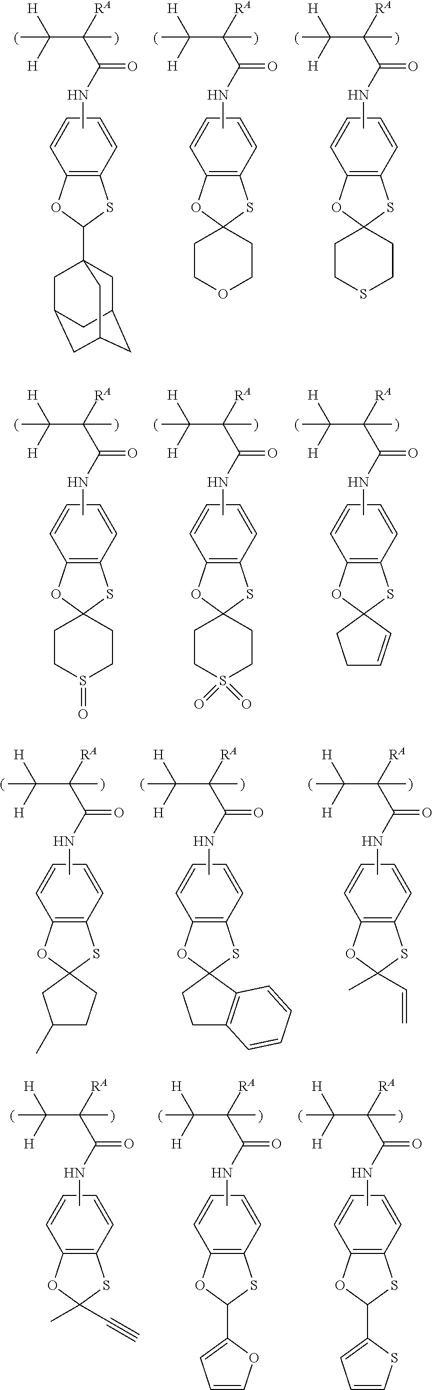

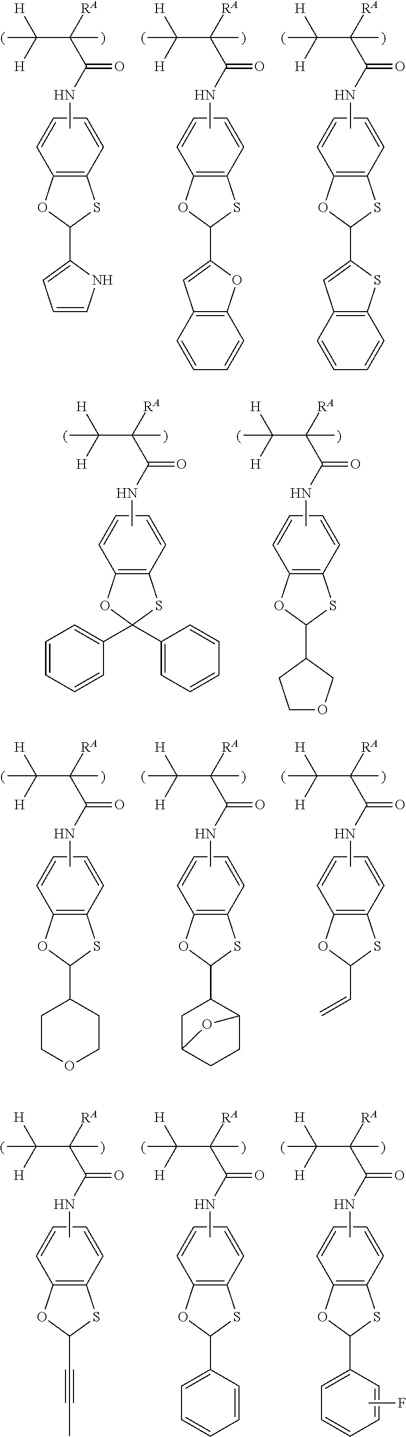

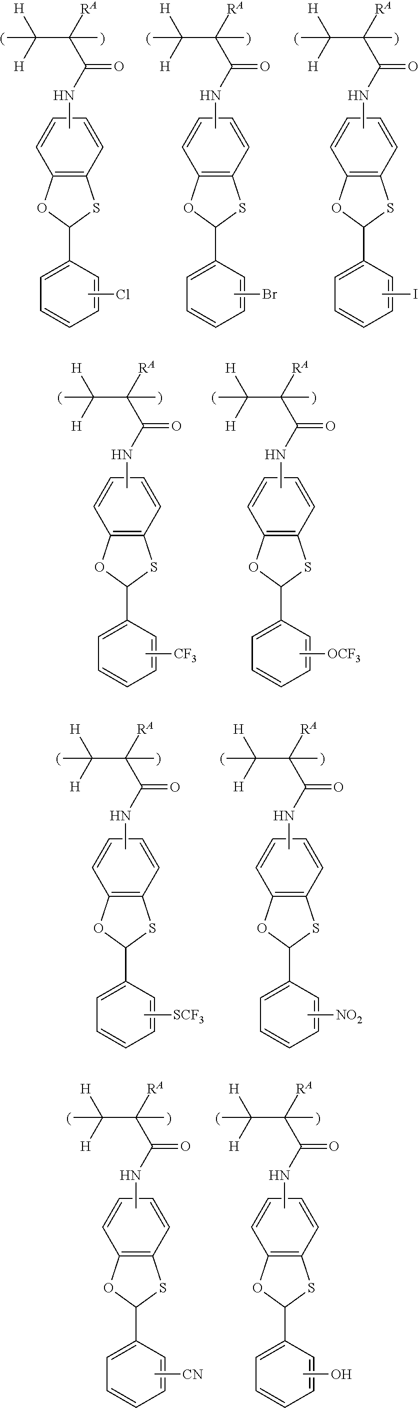

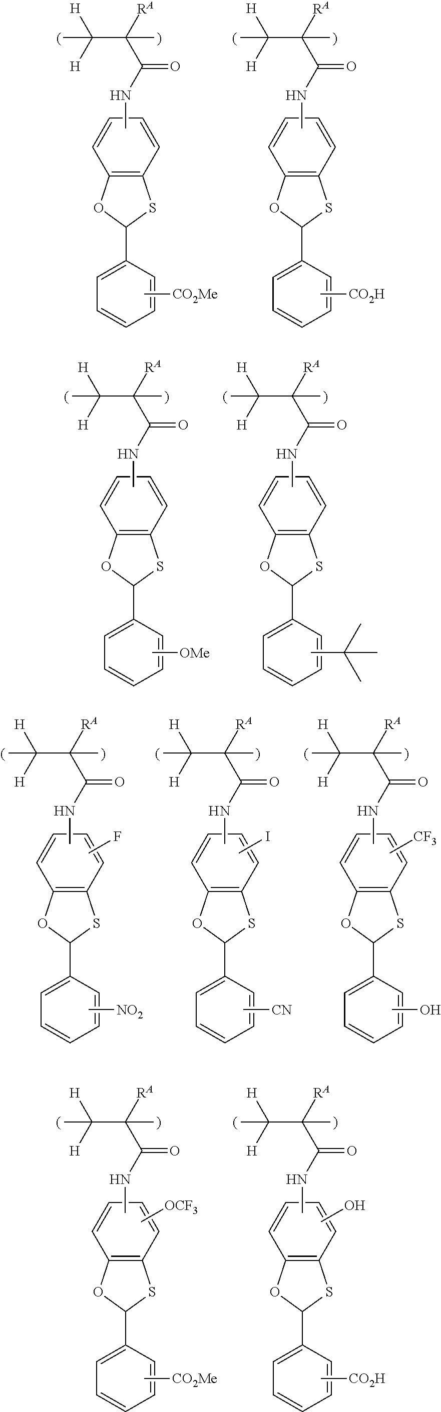

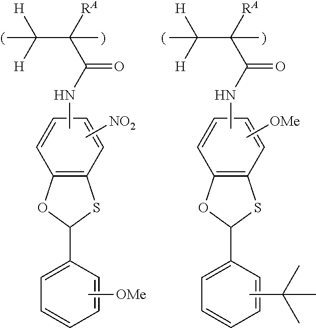

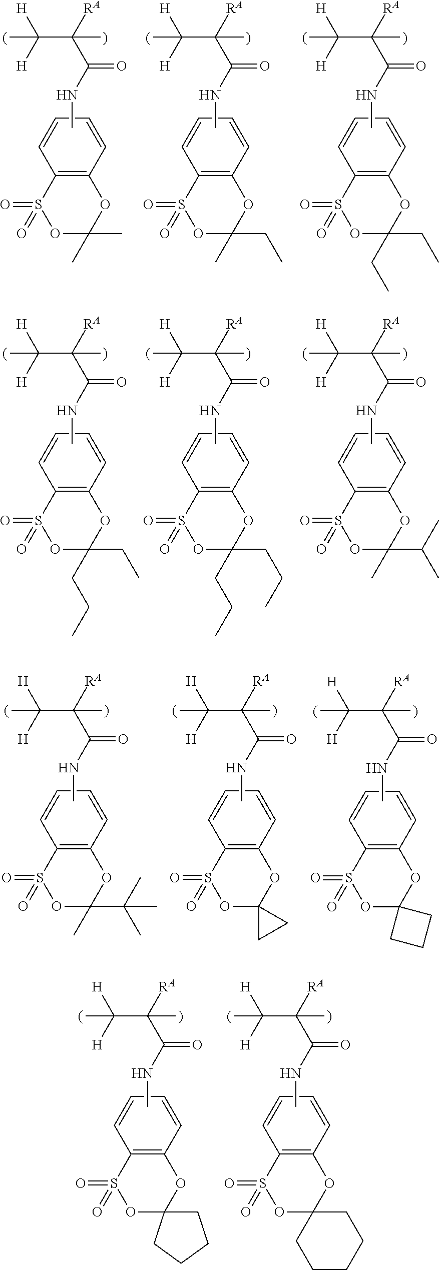

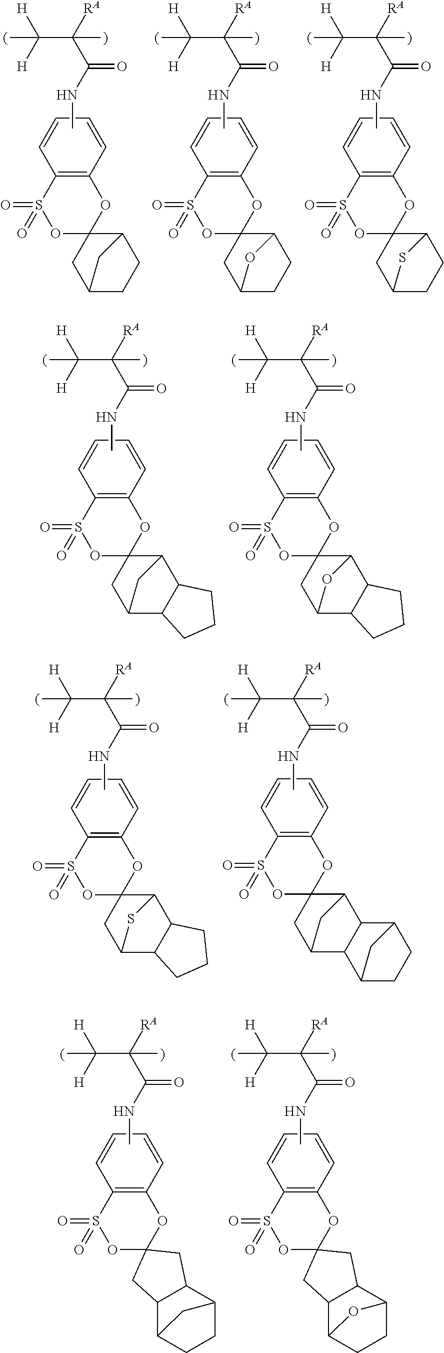

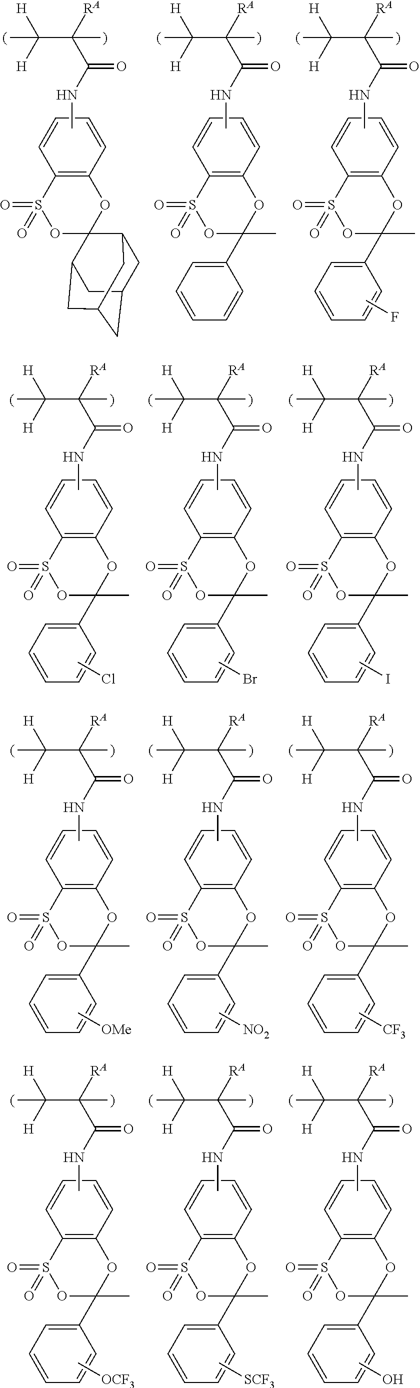

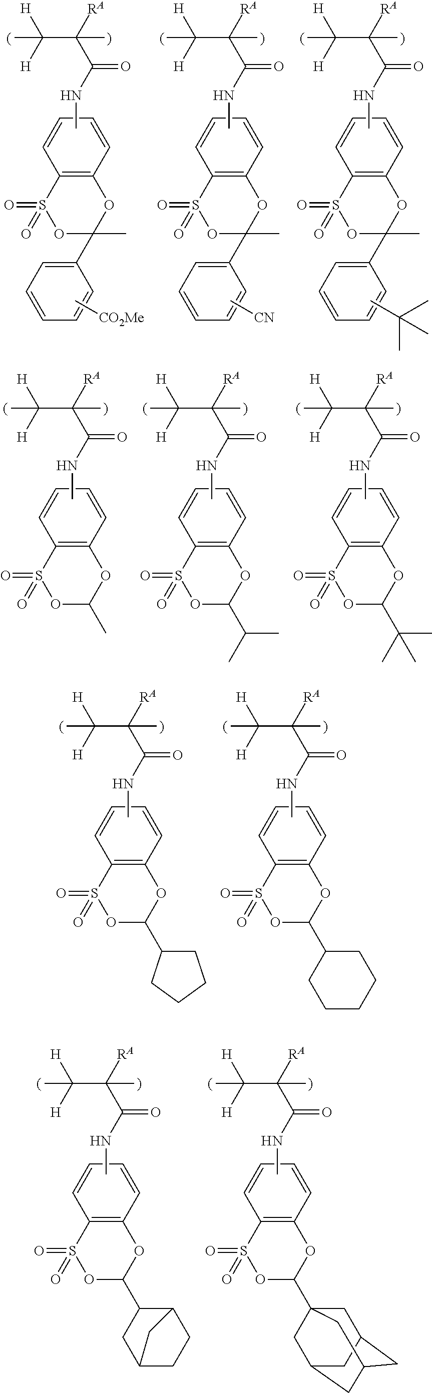

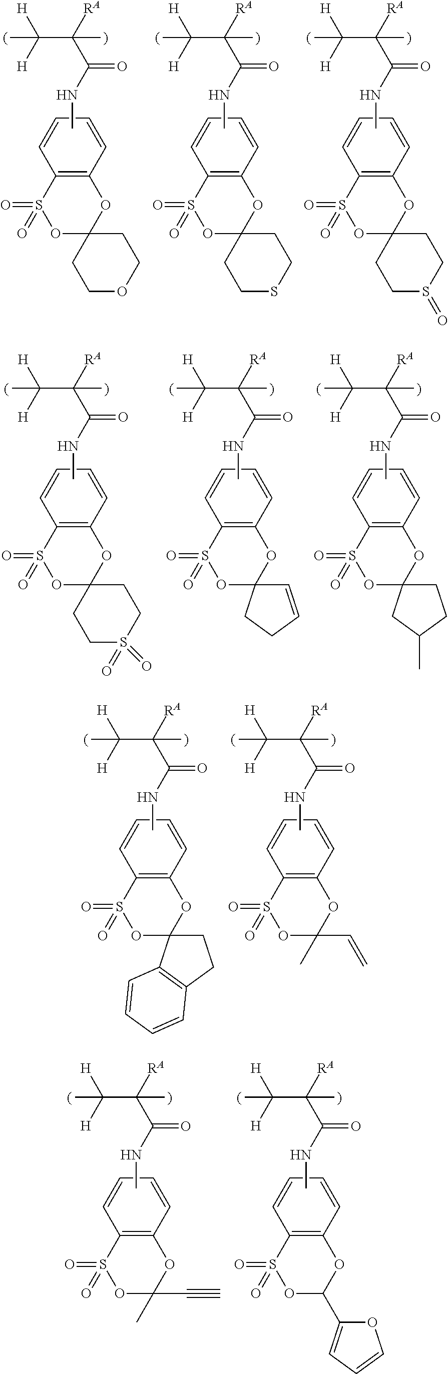

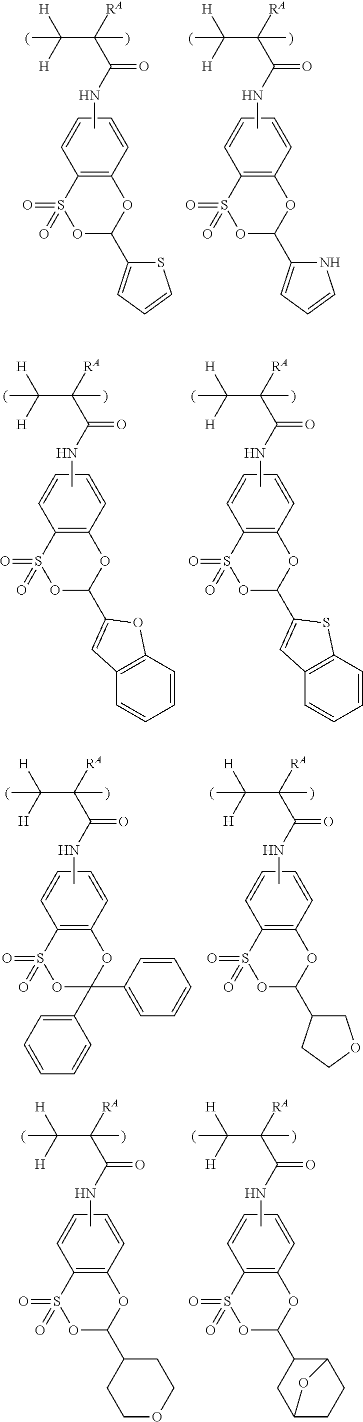

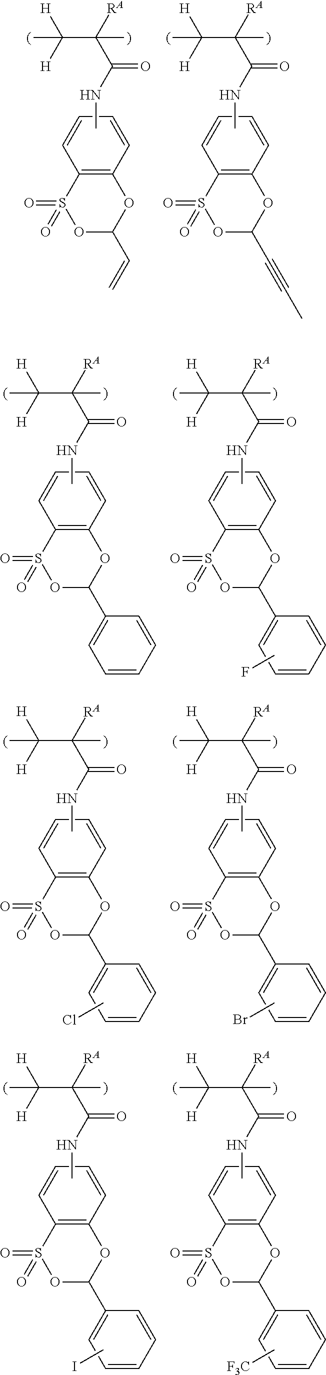

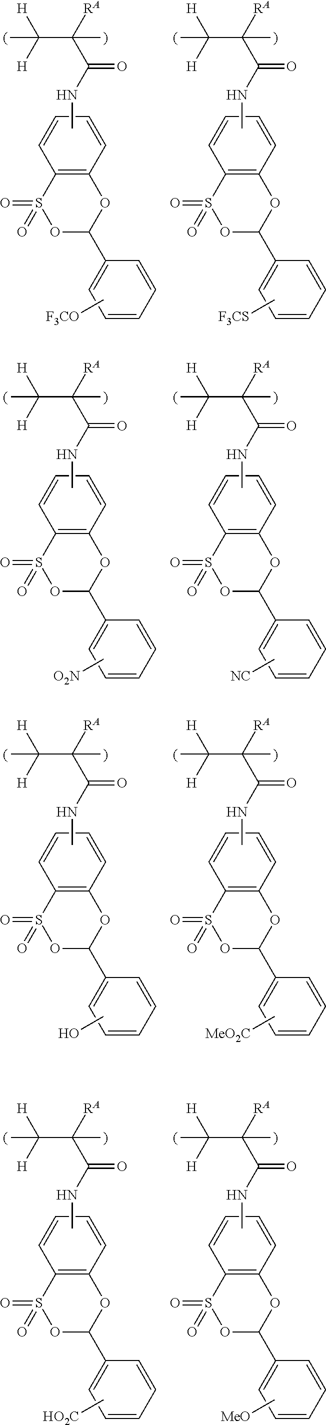

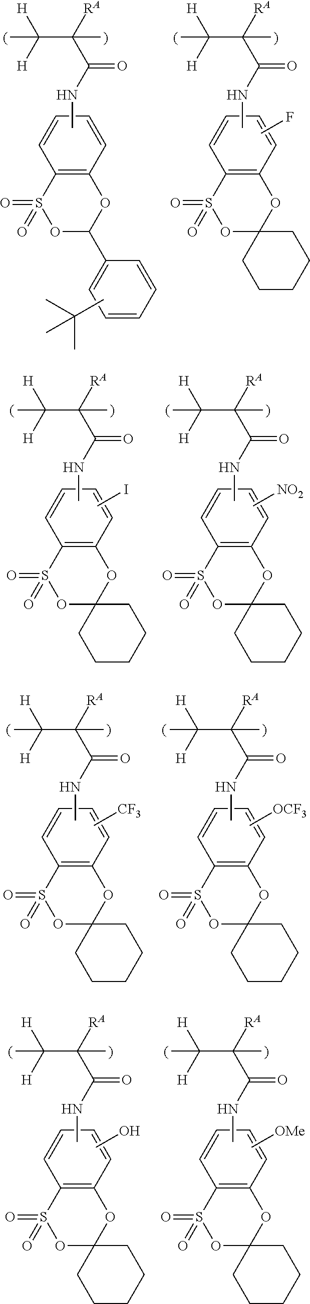

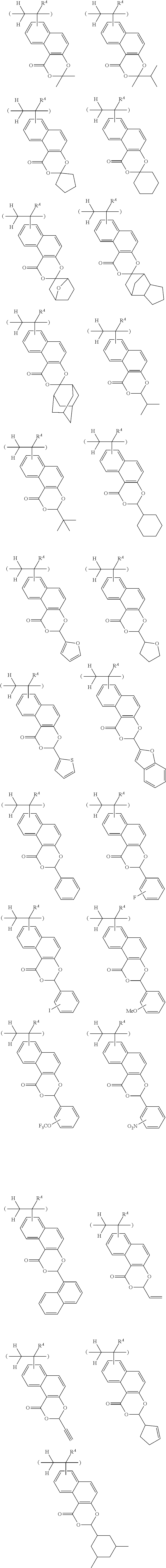

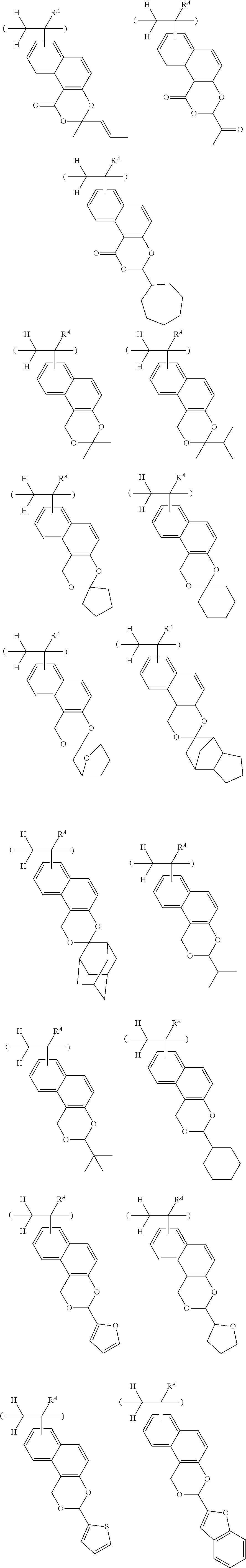

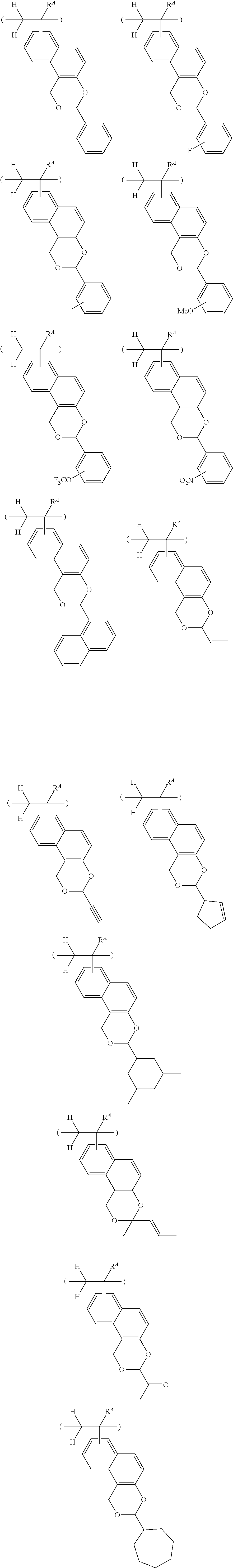

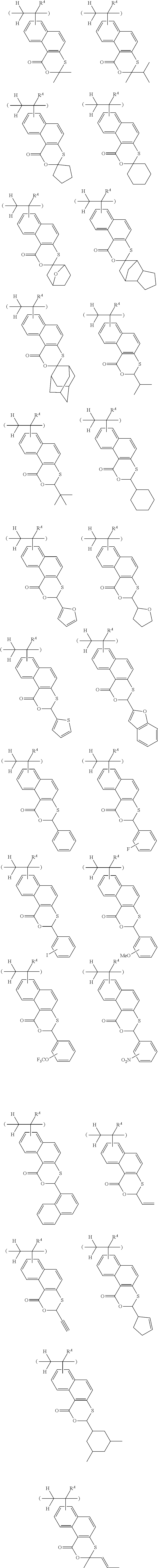

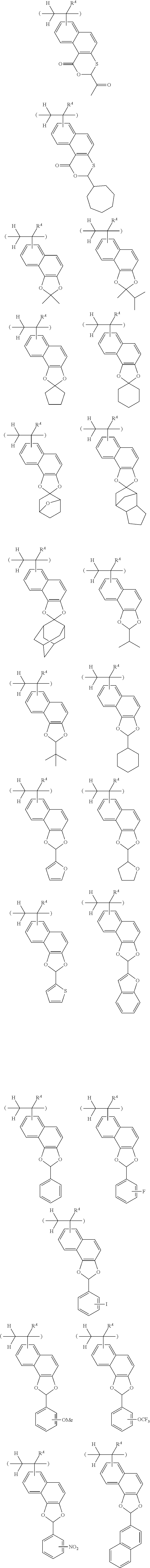

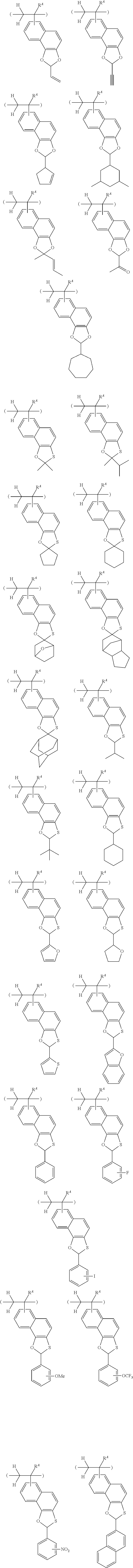

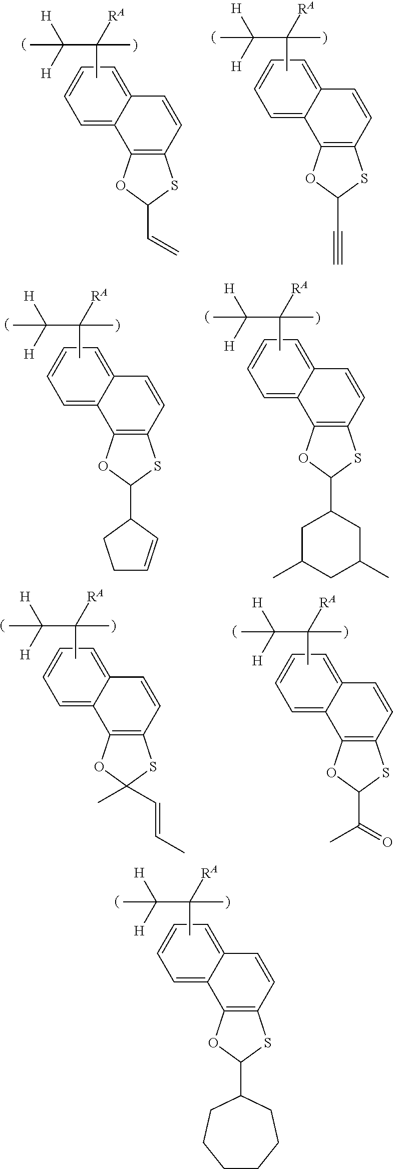

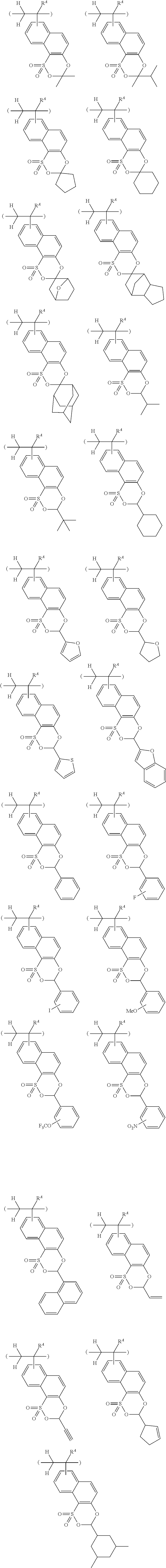

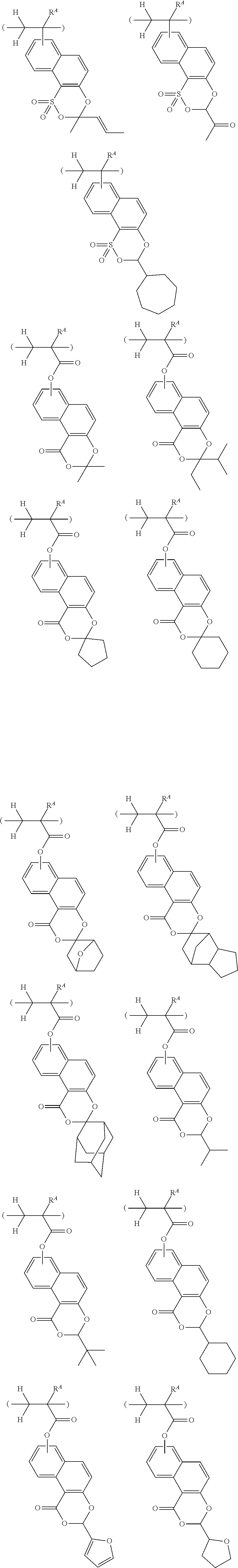

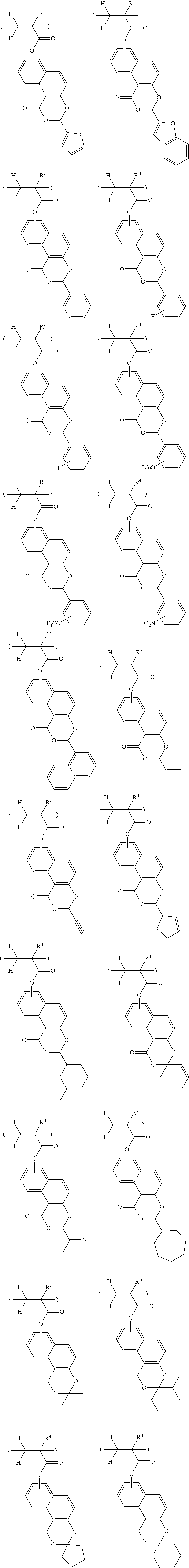

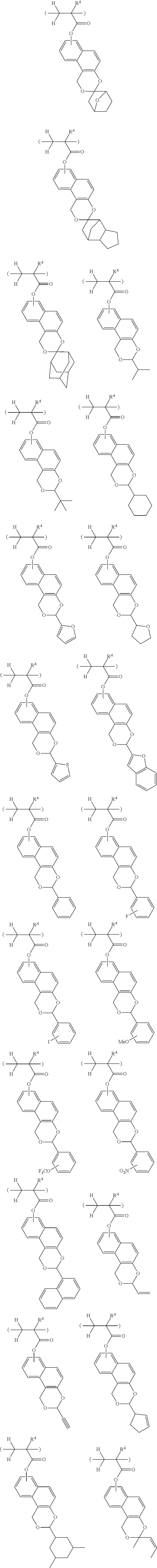

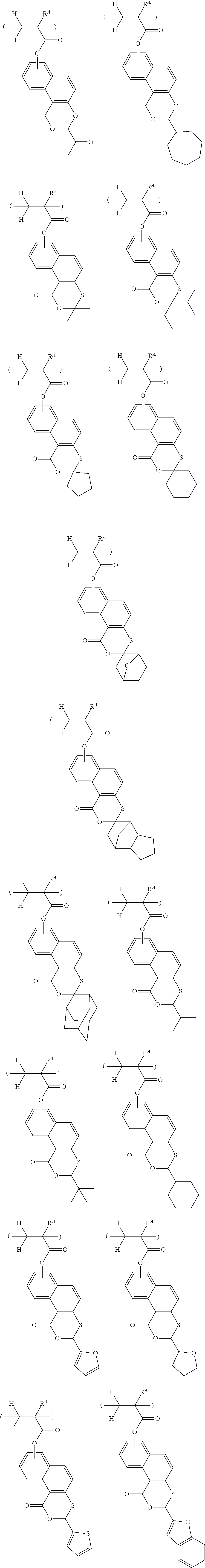

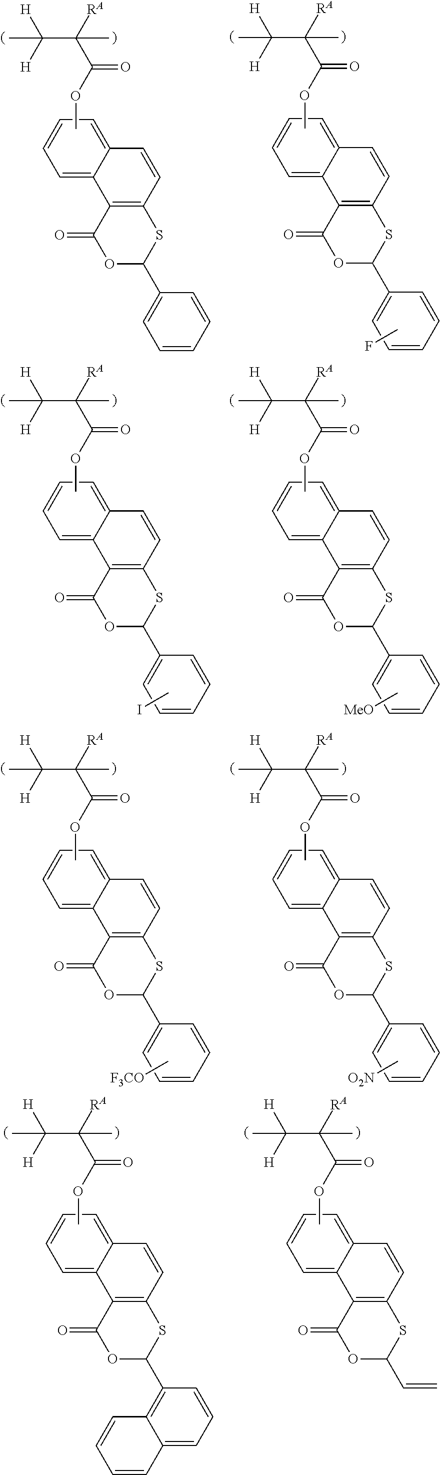

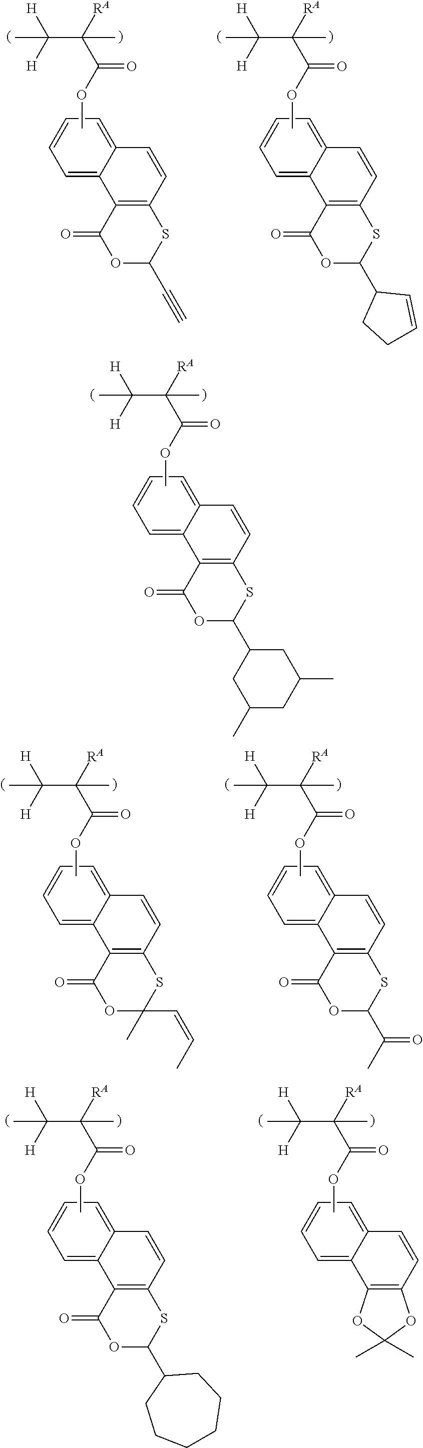

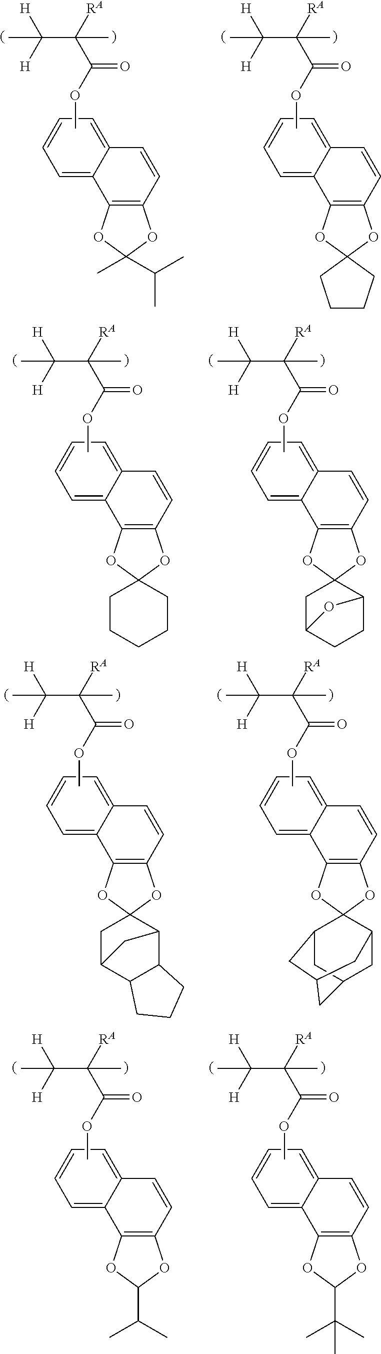

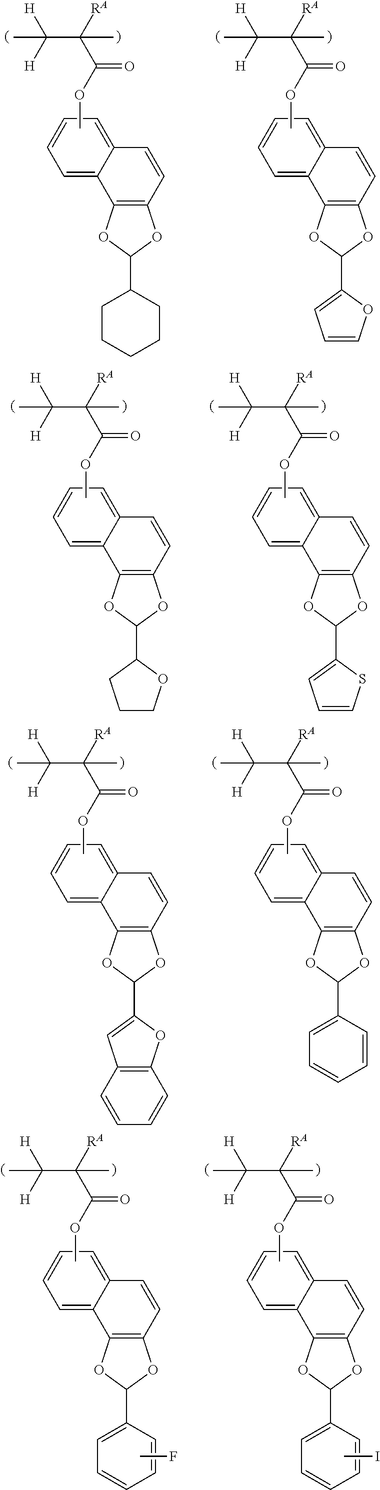

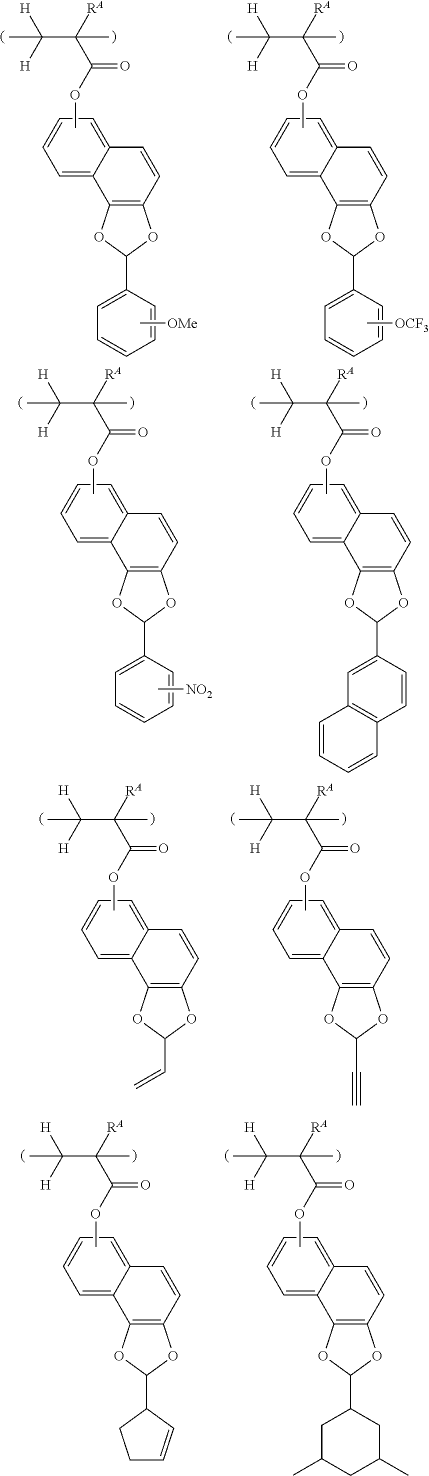

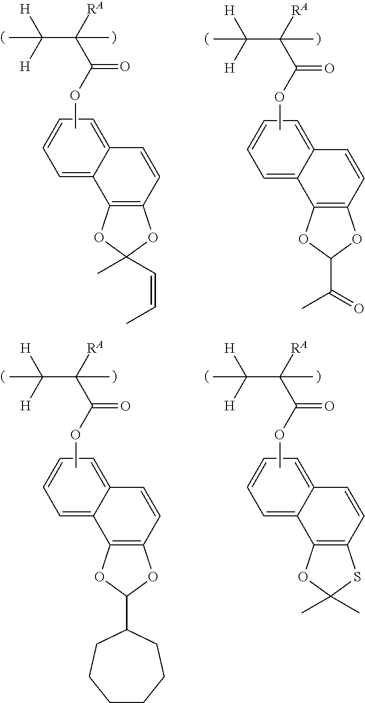

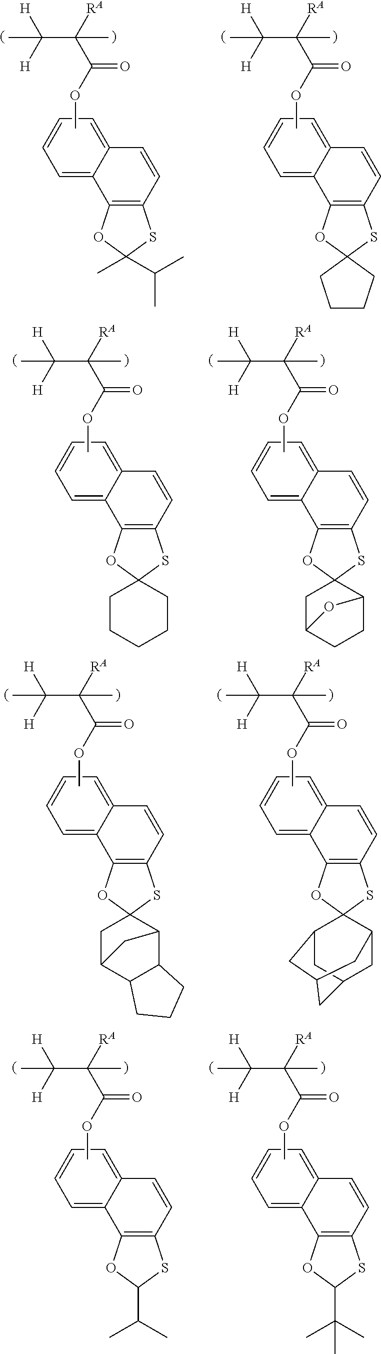

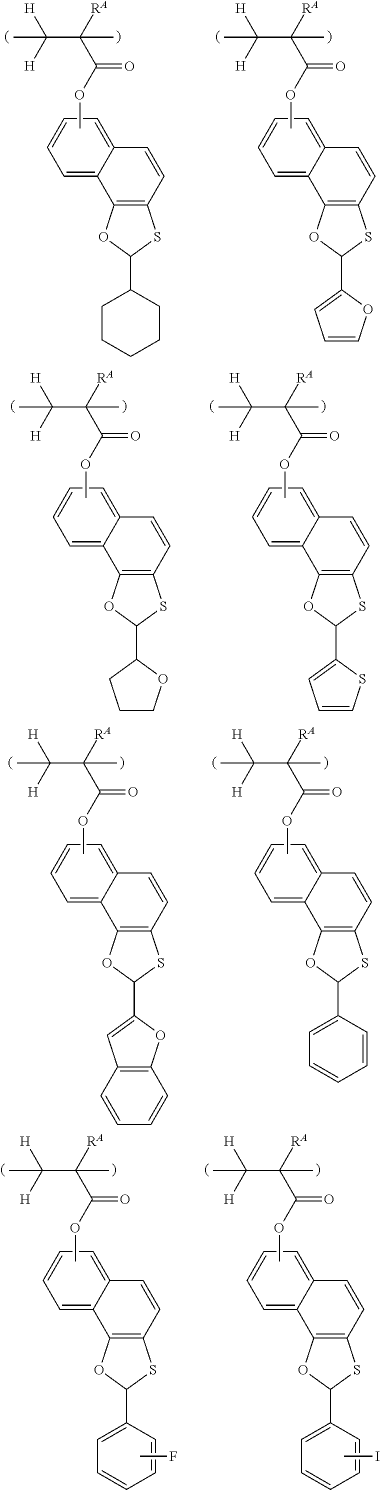

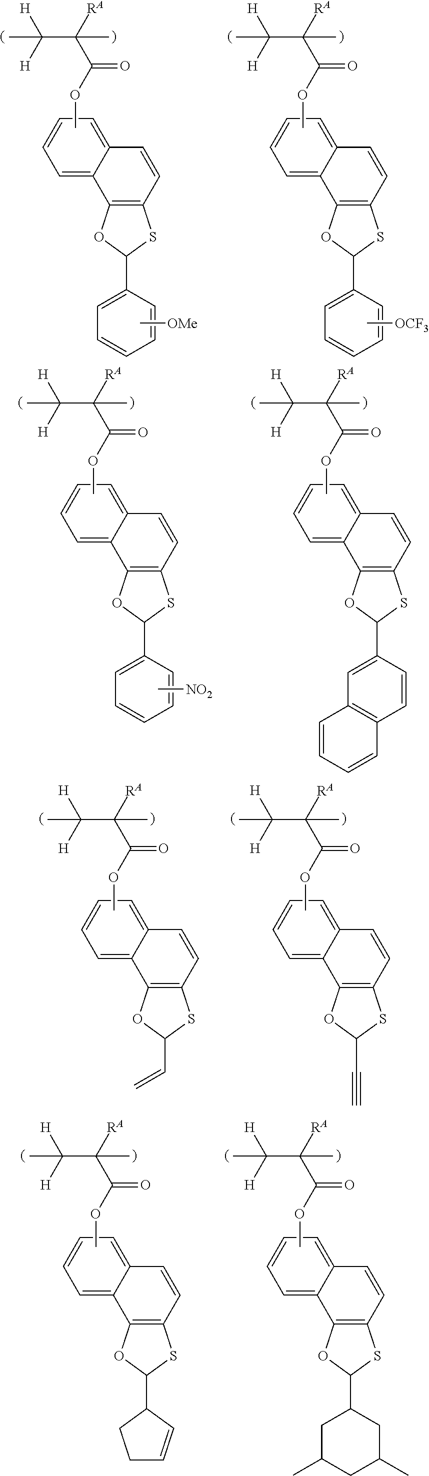

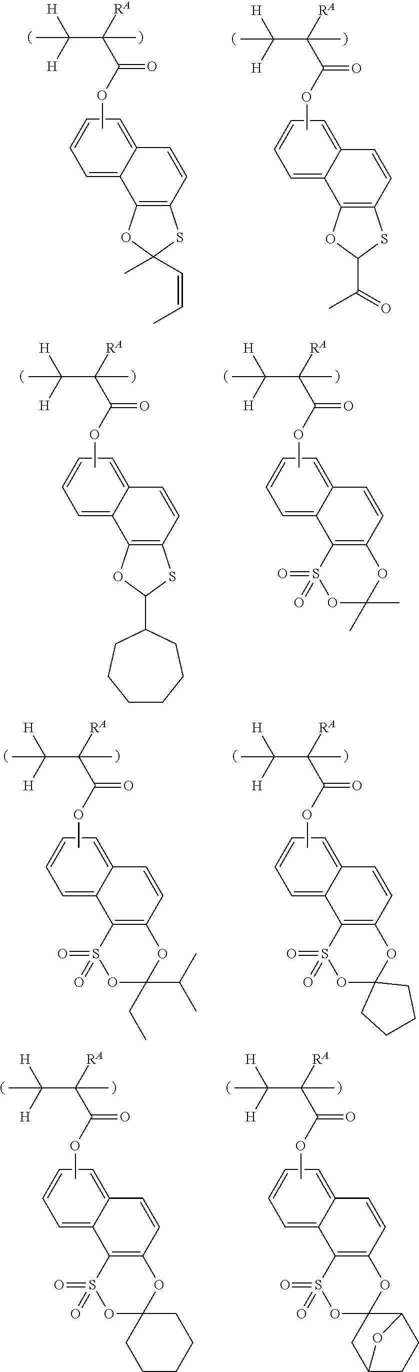

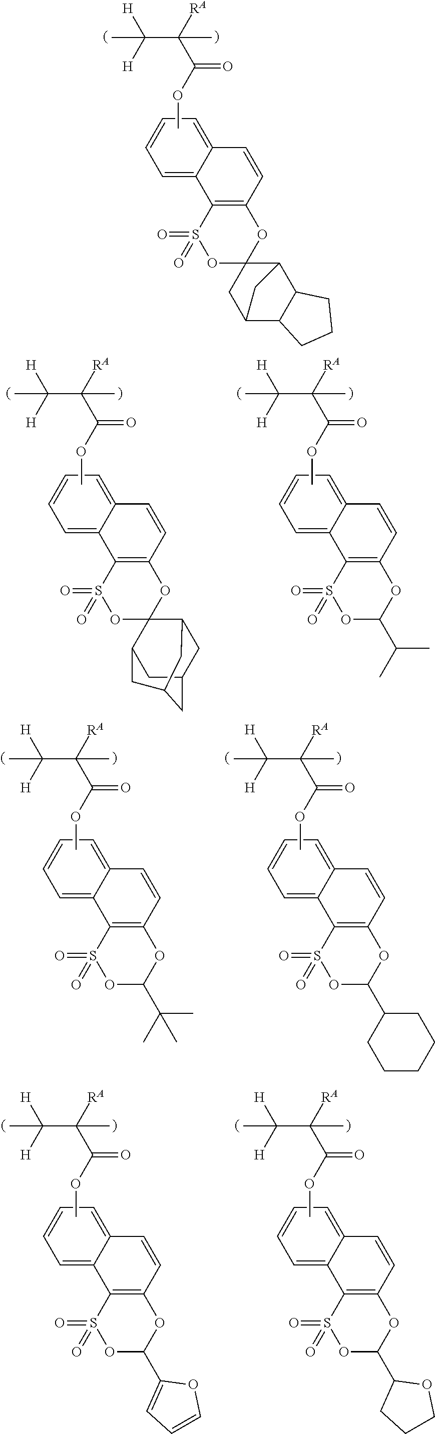

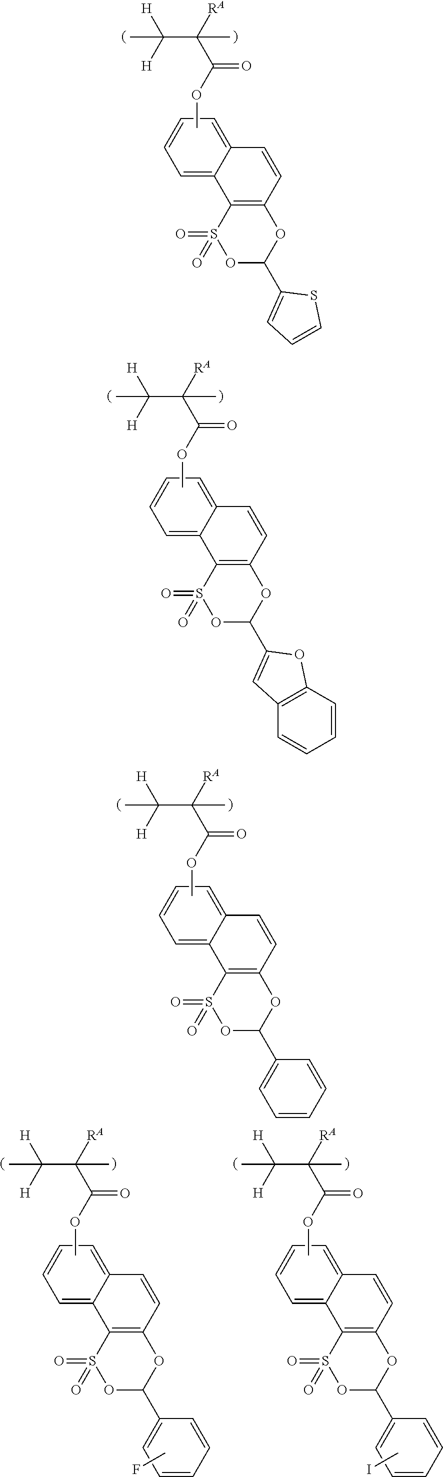

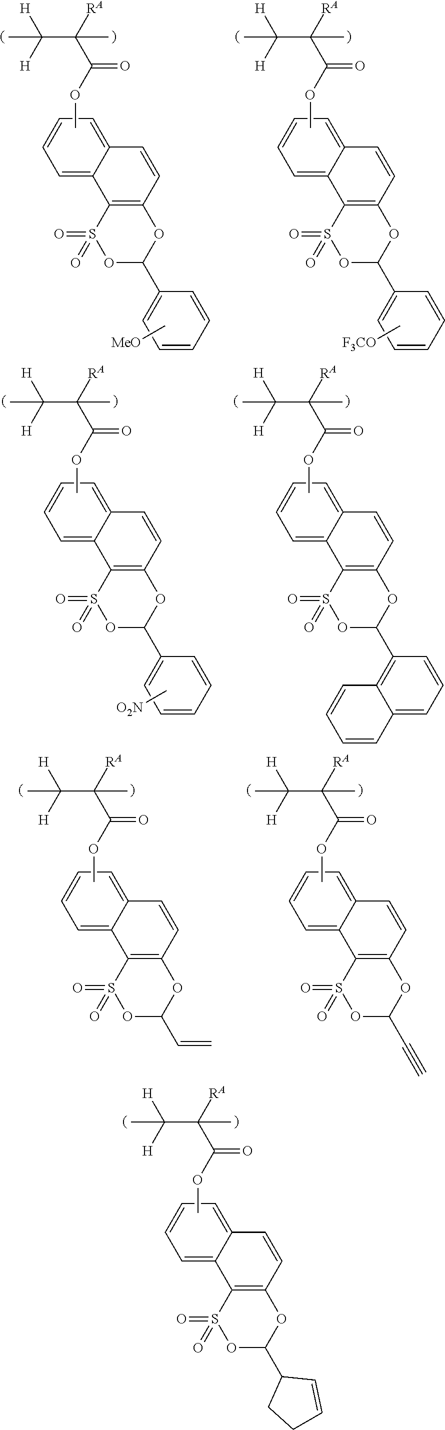

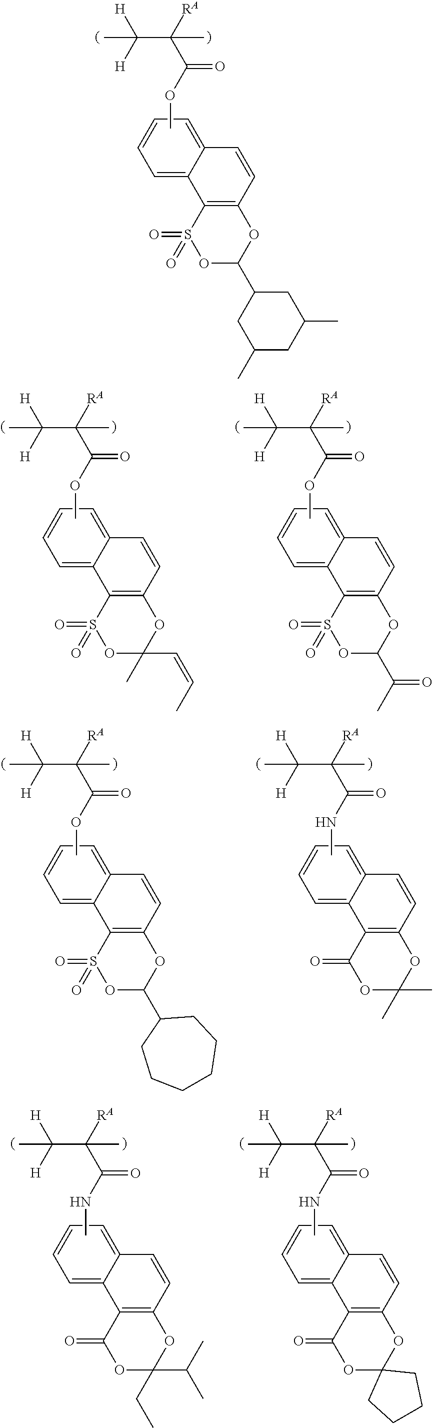

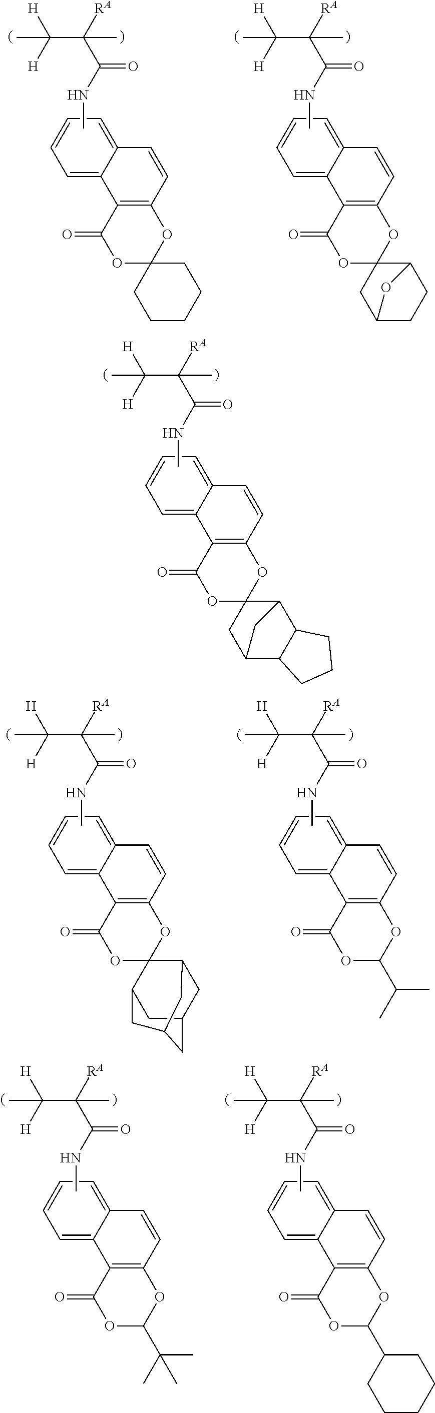

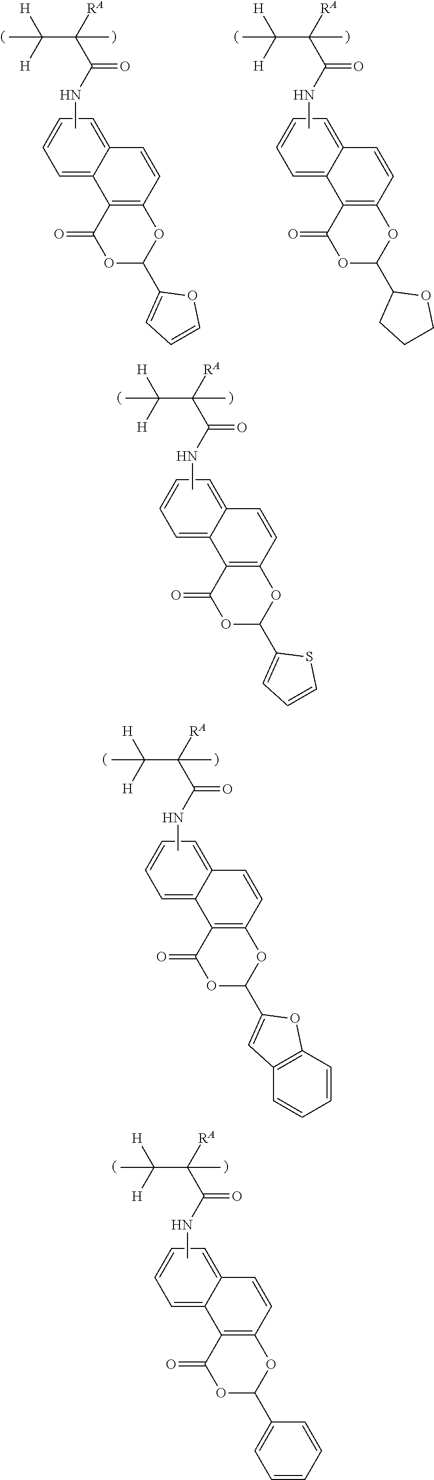

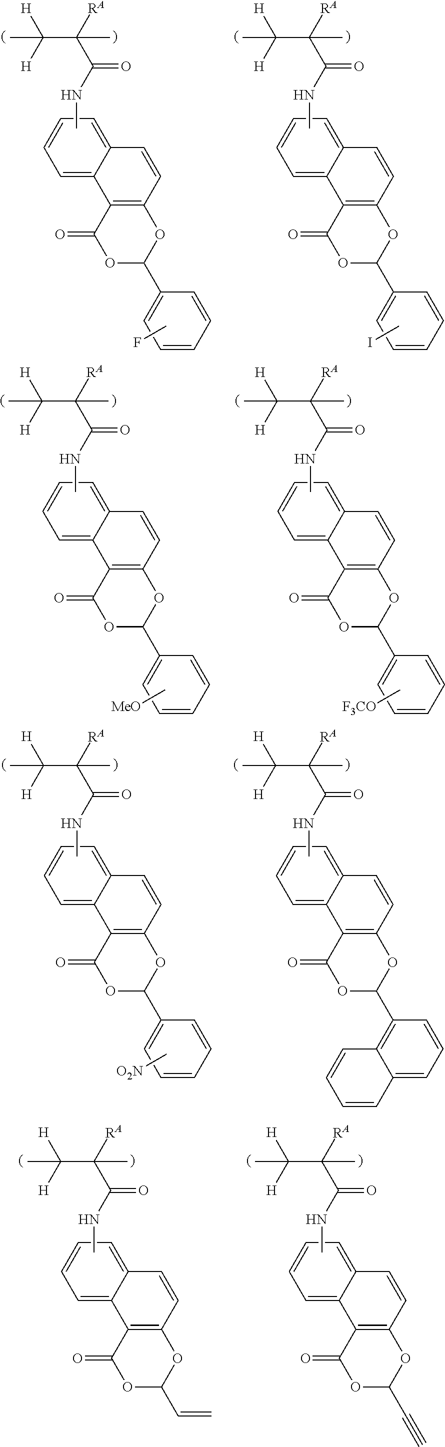

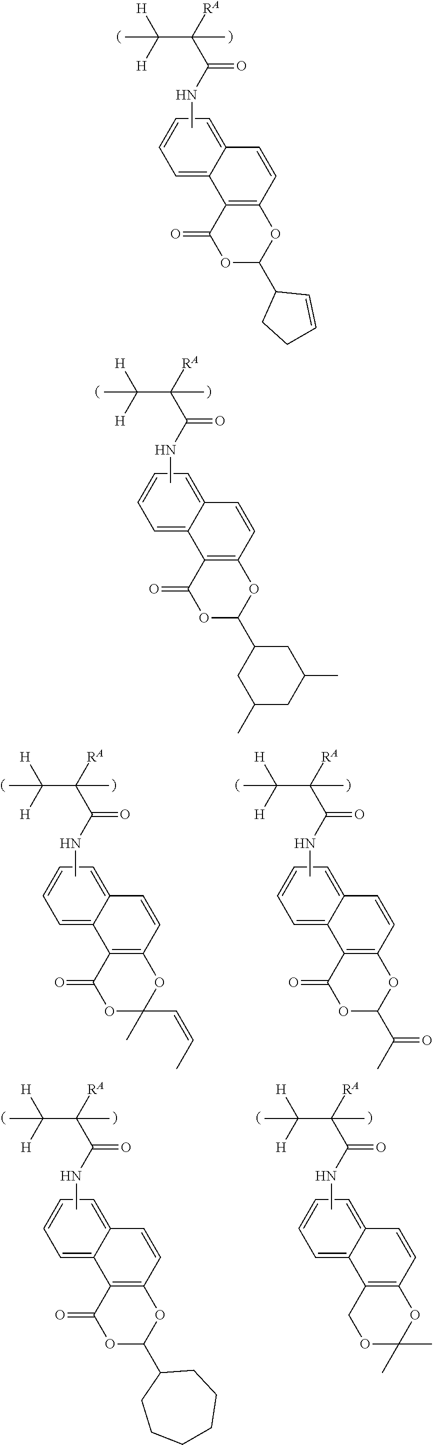

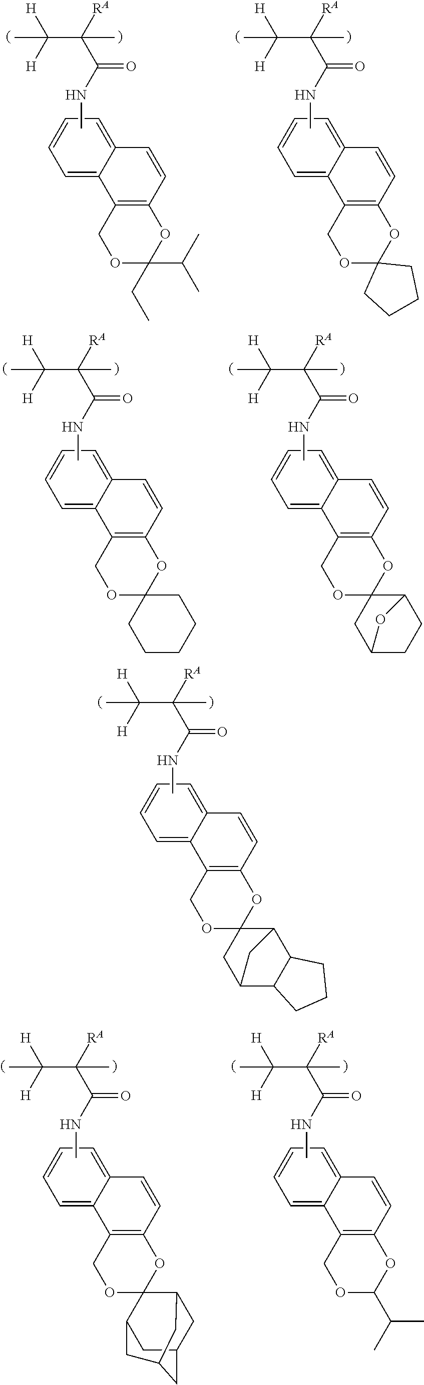

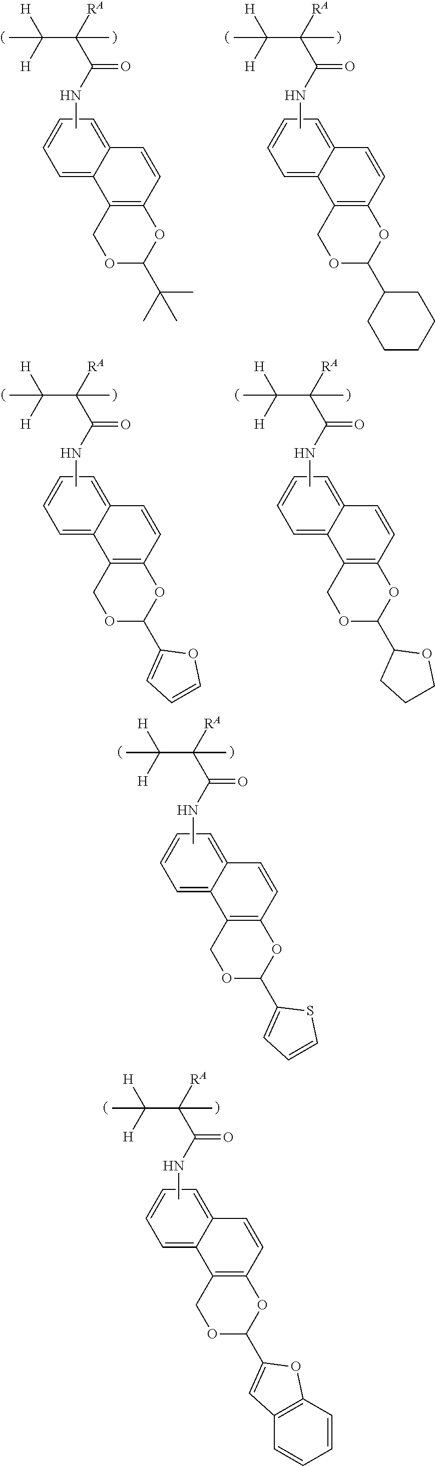

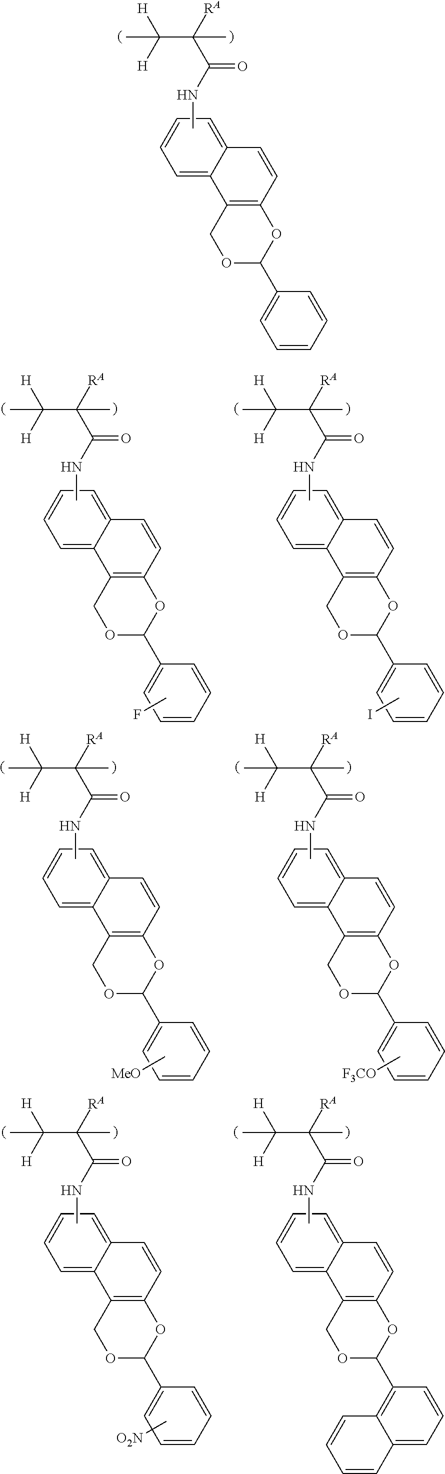

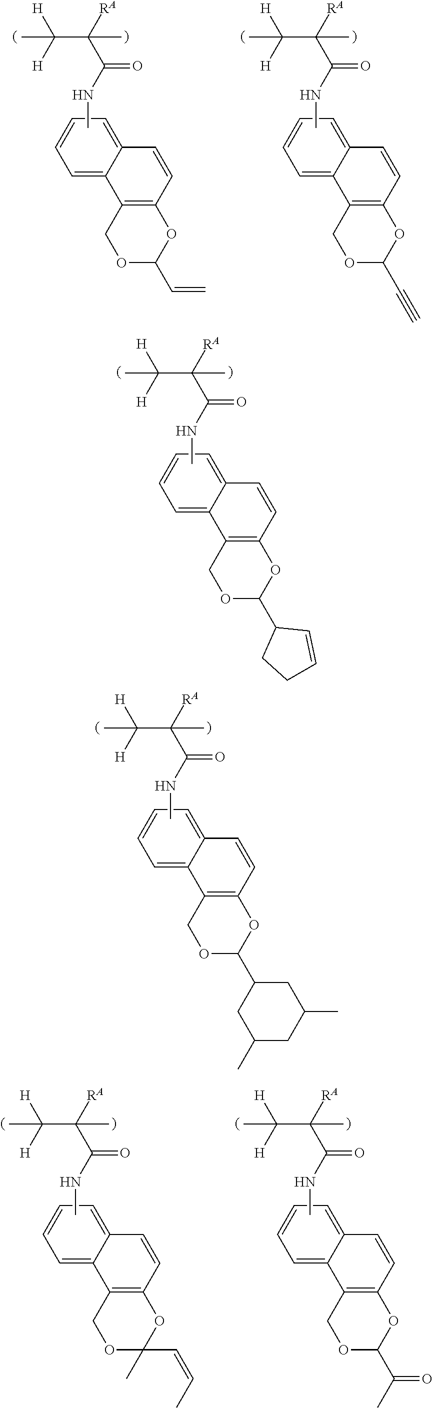

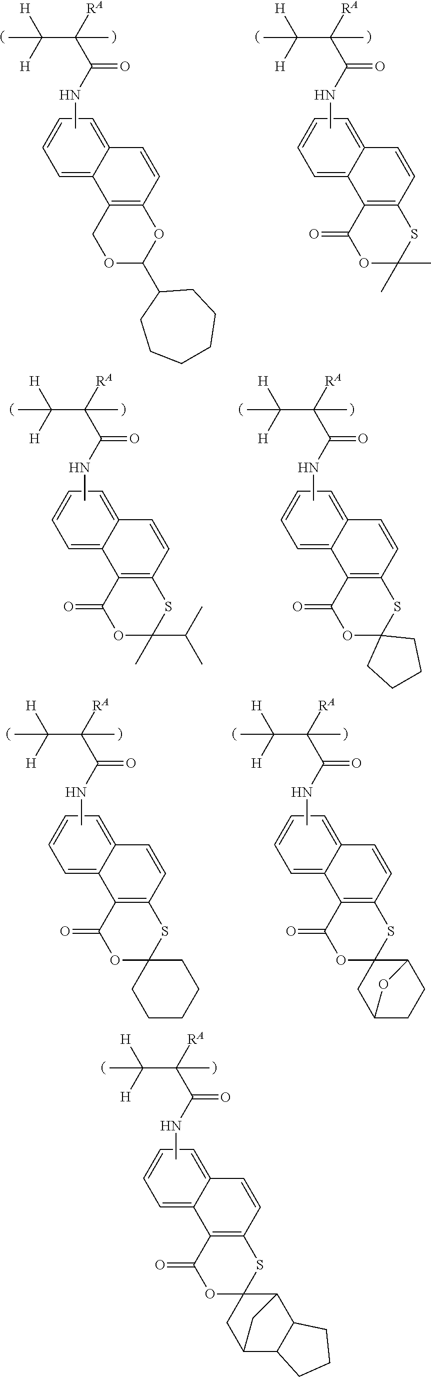

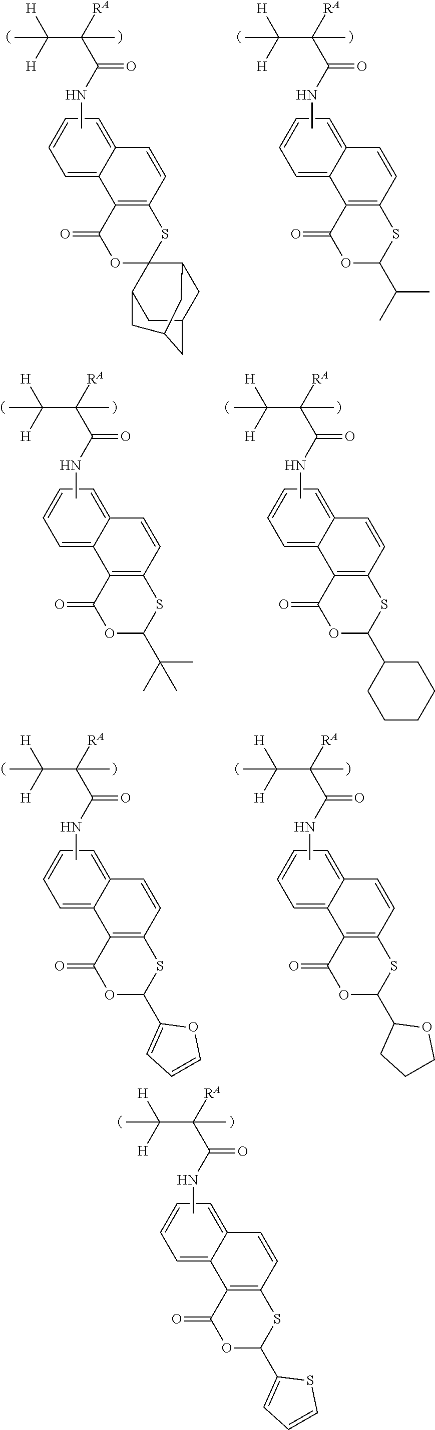

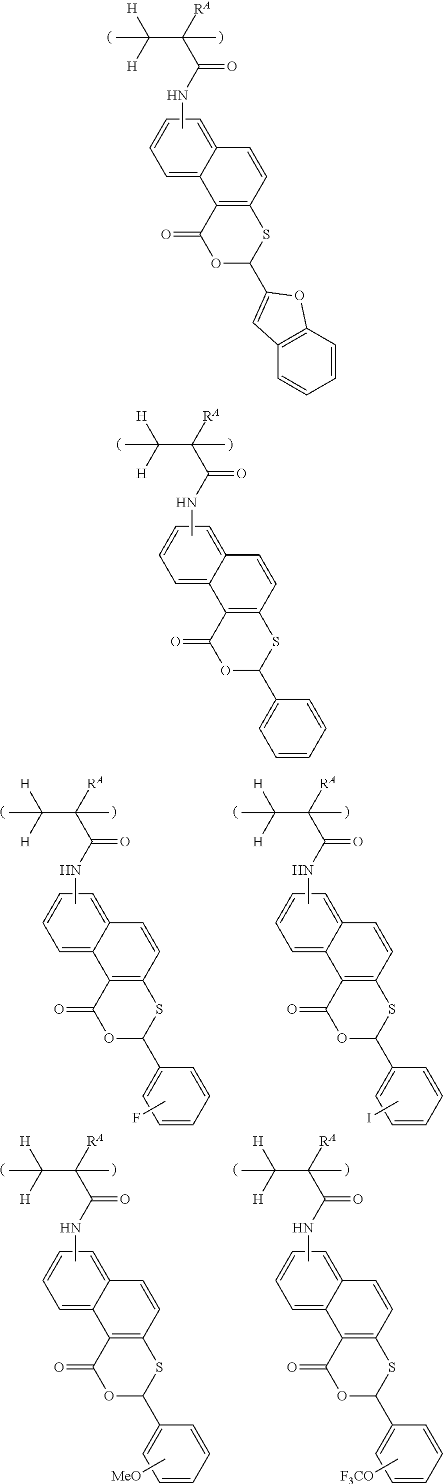

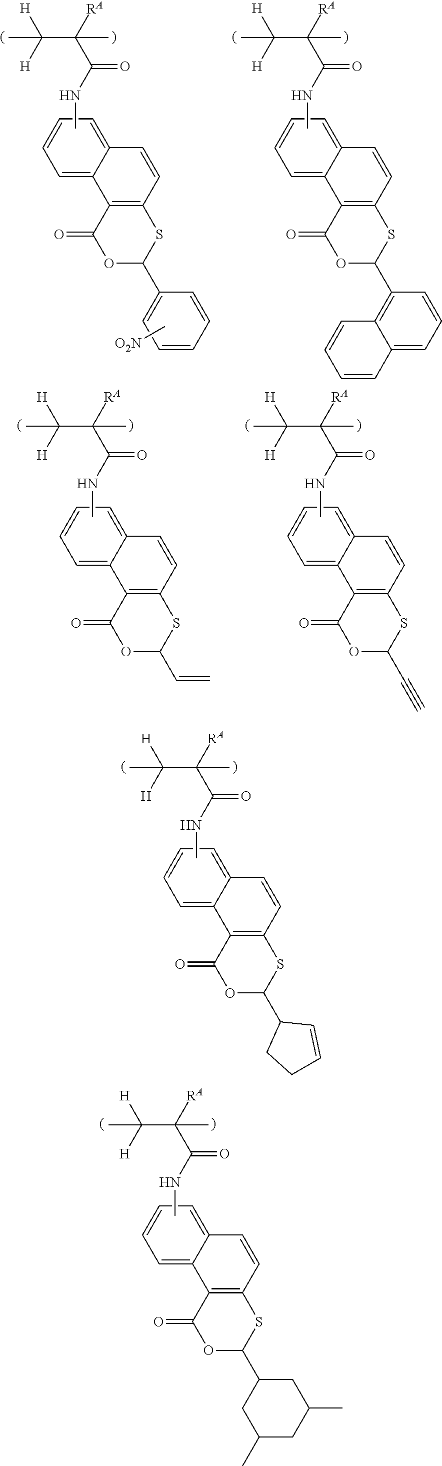

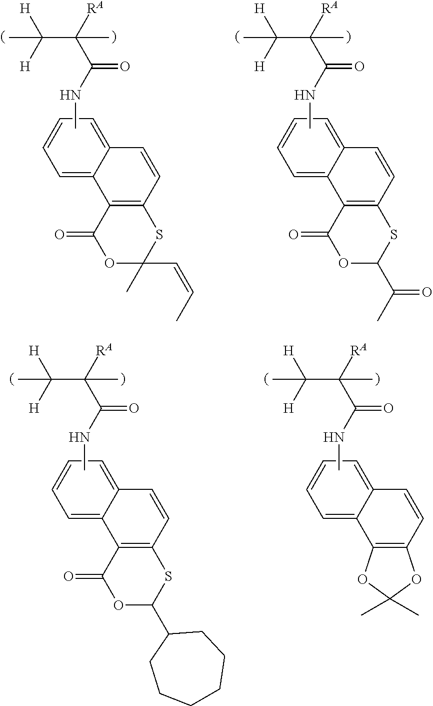

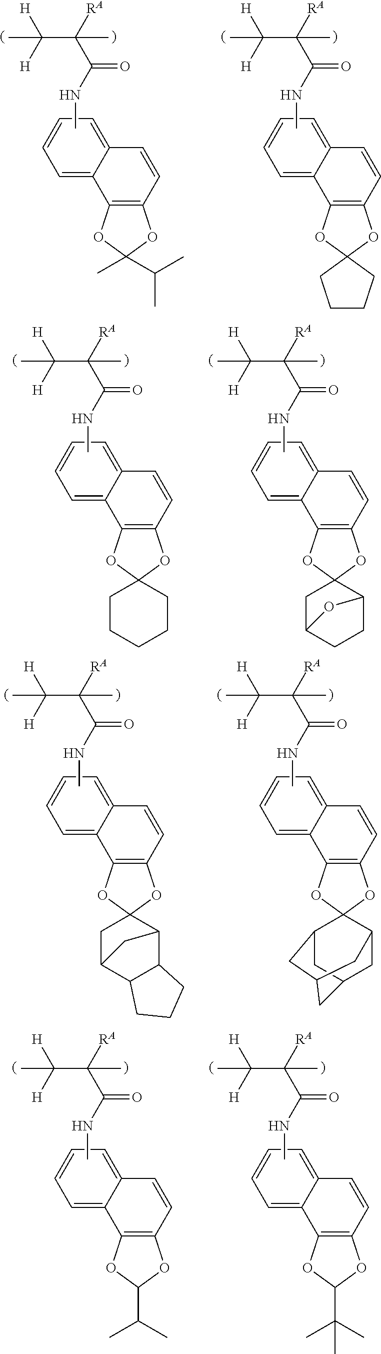

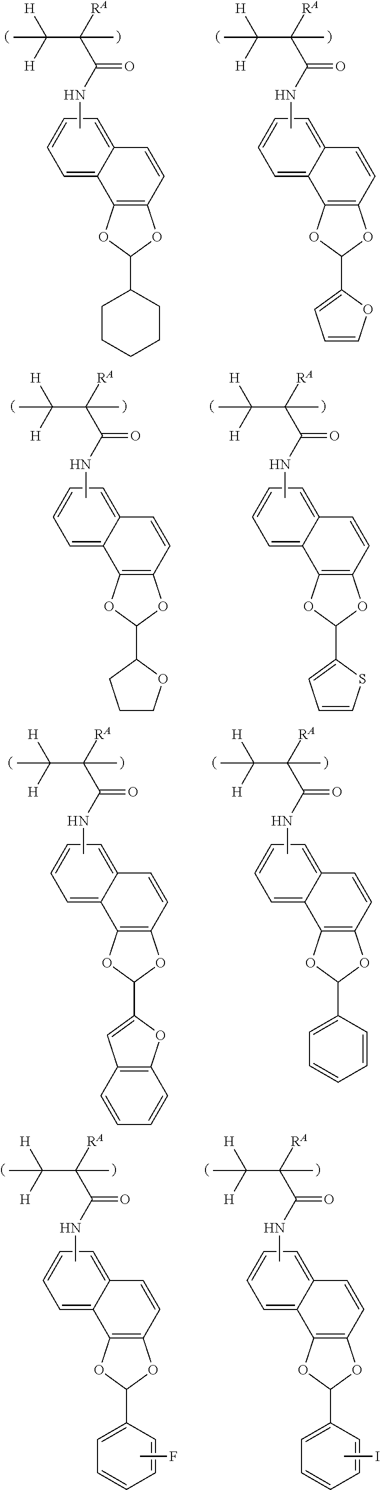

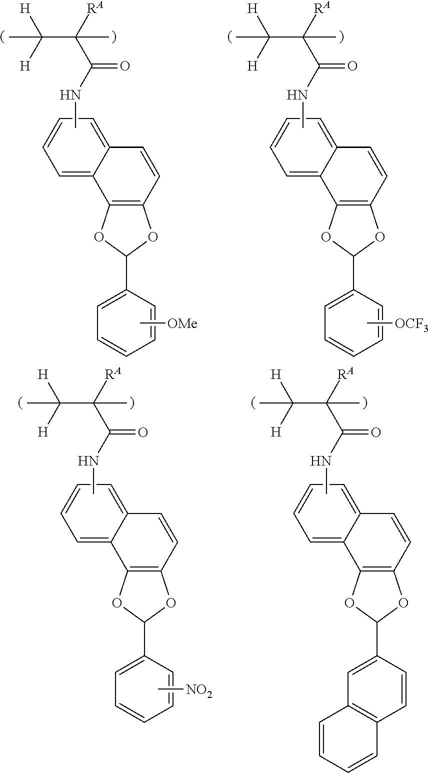

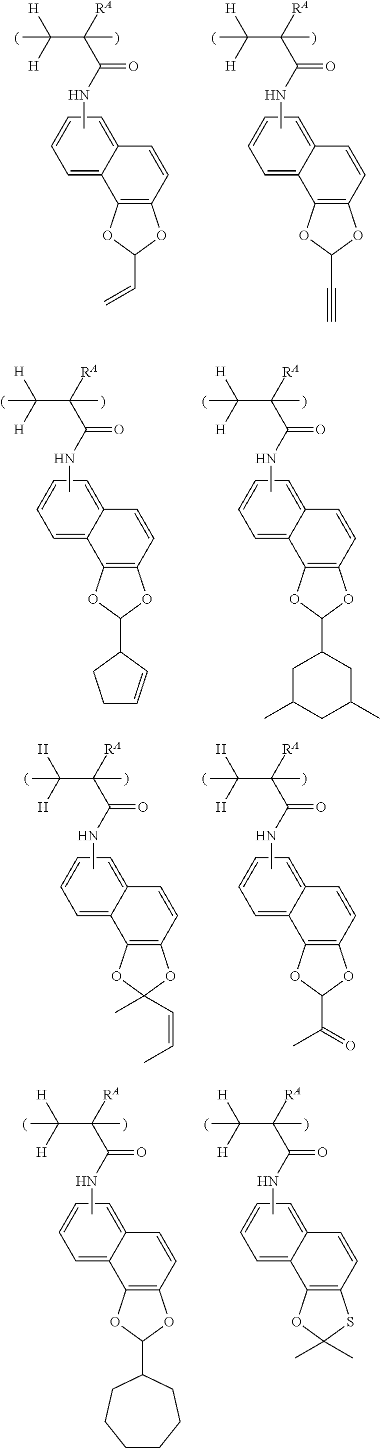

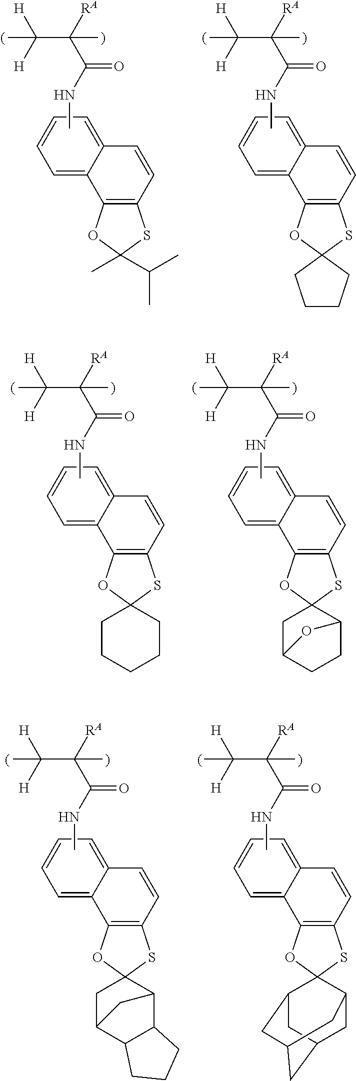

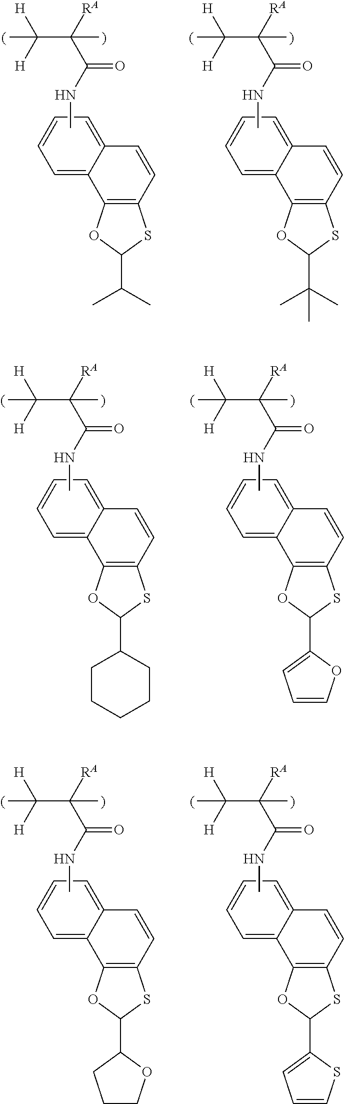

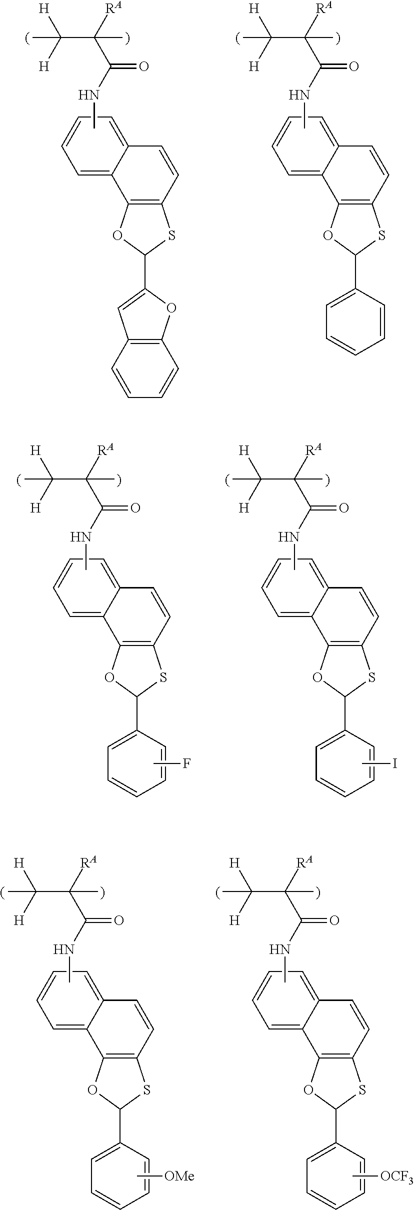

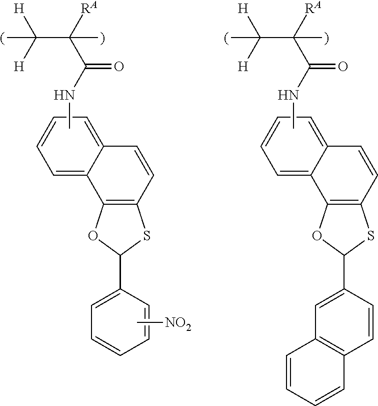

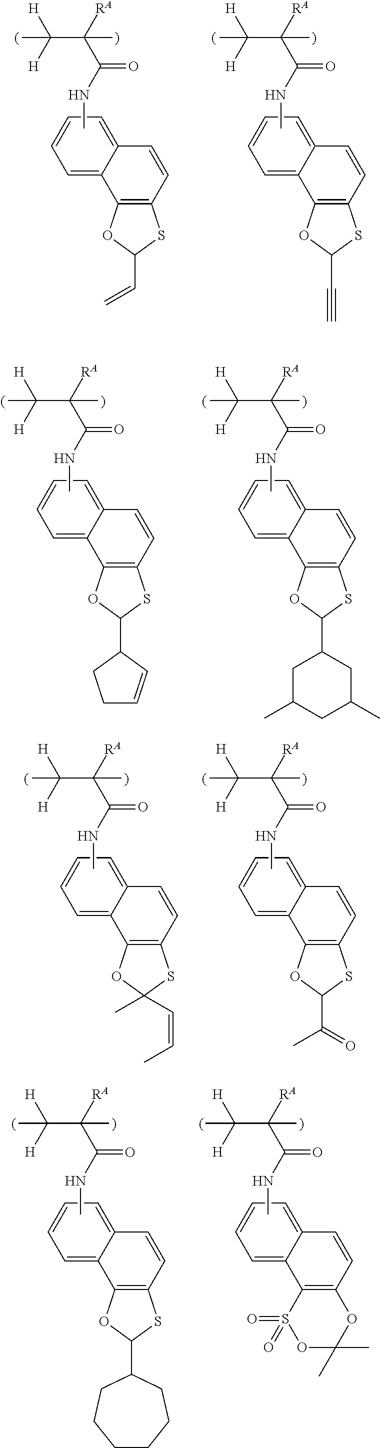

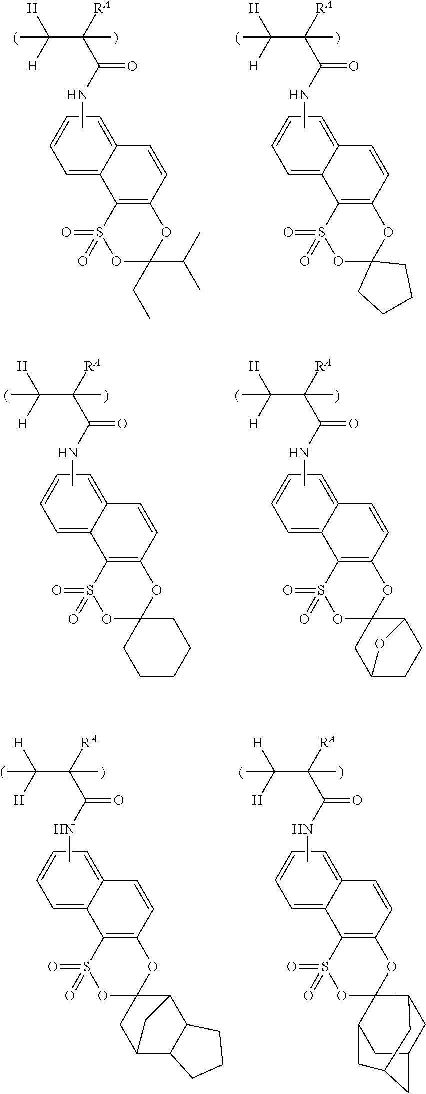

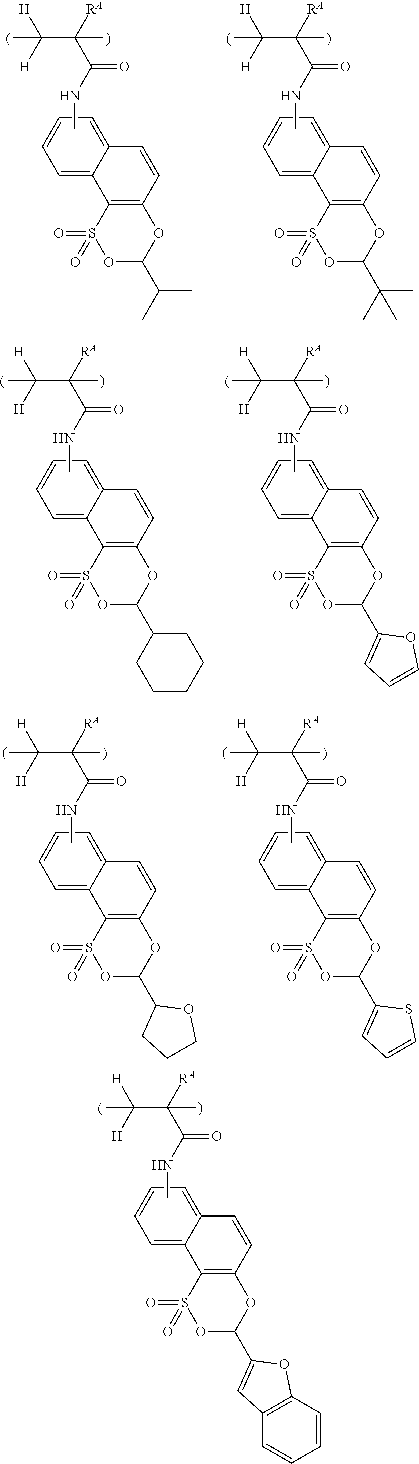

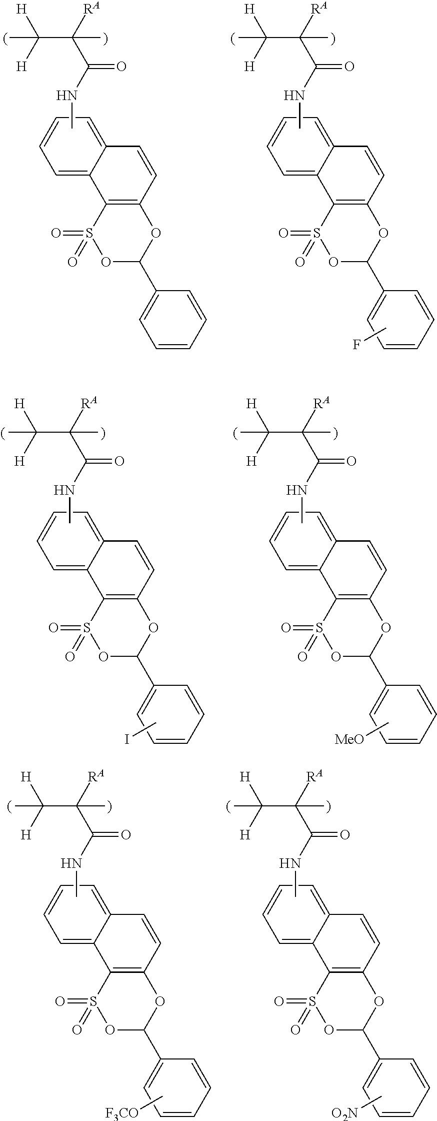

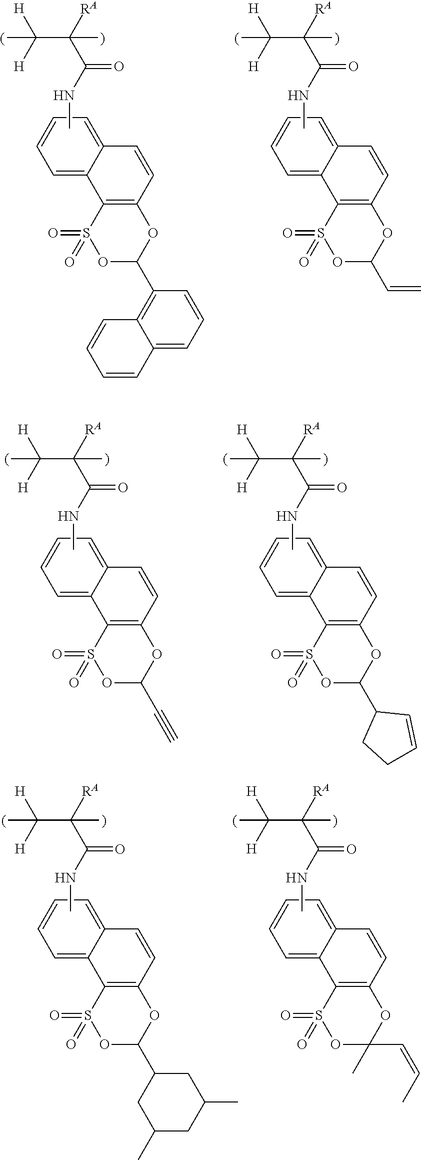

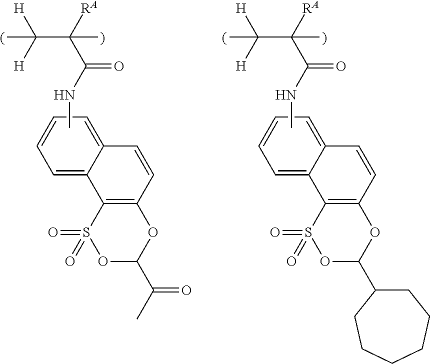

The base polymer may include repeat units having the following formula (b1)(also referred to as repeat units b1, hereinafter) or repeat units having the following formula (b2) (also referred to as repeat units b2, hereinafter).

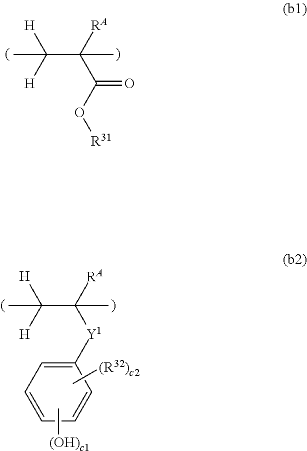

In formulae (b1) and (b2), R^{A} is each independently a hydrogen atom, a fluorine atom, a methyl group, or a trifluoromethyl group. Y¹ is a single bond or *—C(═O)—O—. * designates a point of attachment to the carbon atom in the backbone. R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—). R³² is a halogen atom, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. When c2 is 2, 3, or 4, respective R³² may be the same as or different from each other. cl is 1, 2, 3, or 4. c2 is 0, 1, 2, 3, or 4. Provided that 1≤c1+c2≤5.

Specific examples of the repeat units bl are shown below, but not limited thereto. In the following formula. R^{A} is as defined above.

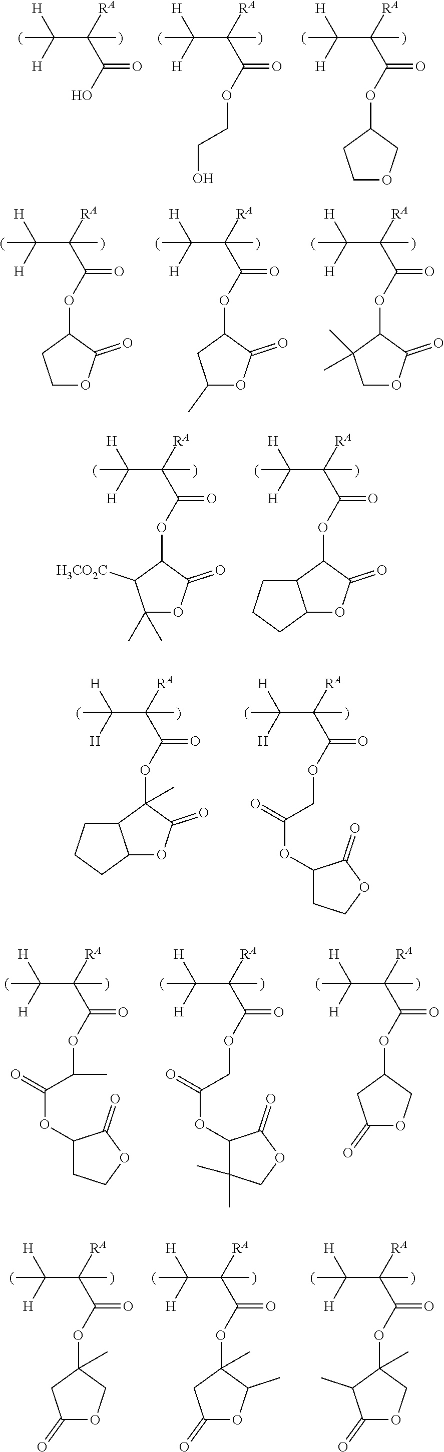

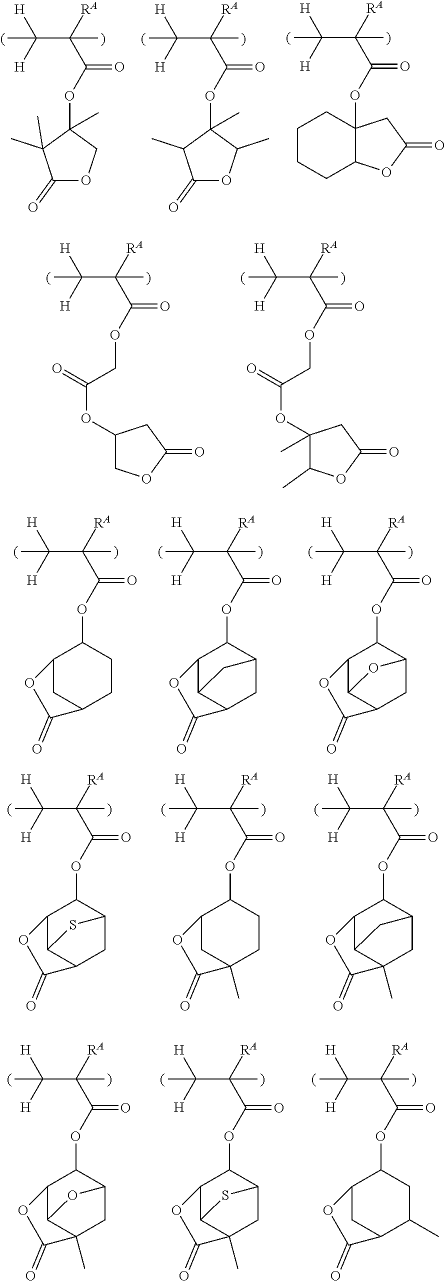

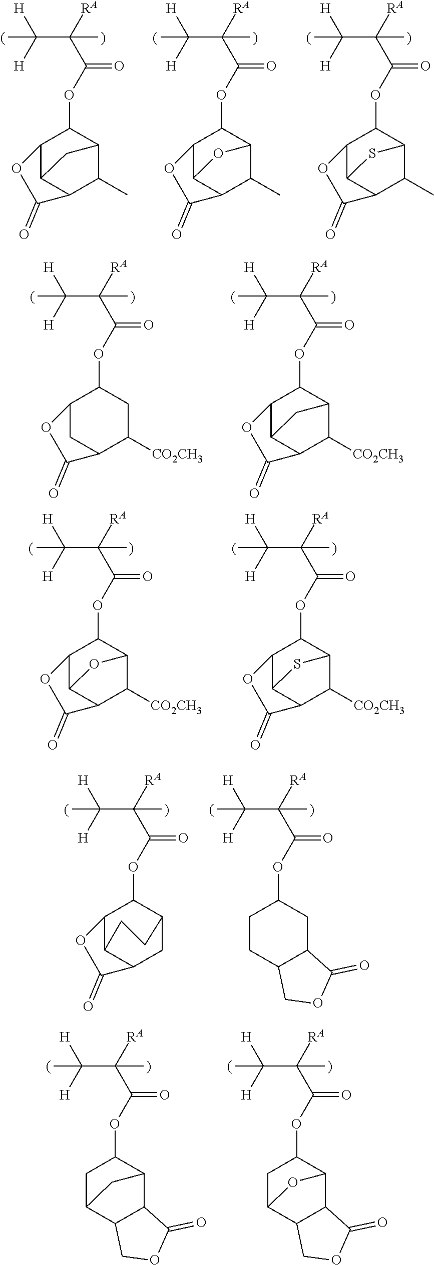

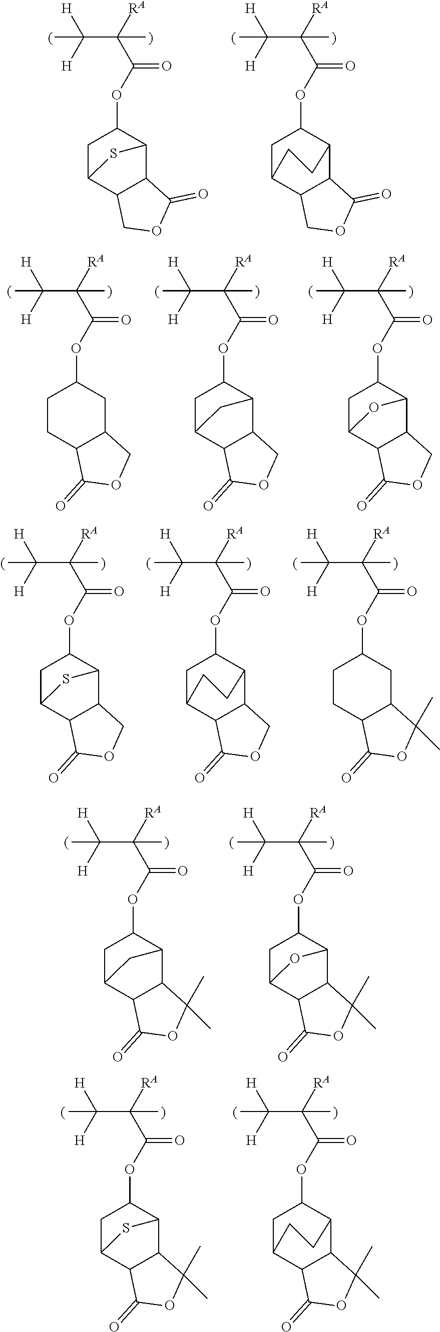

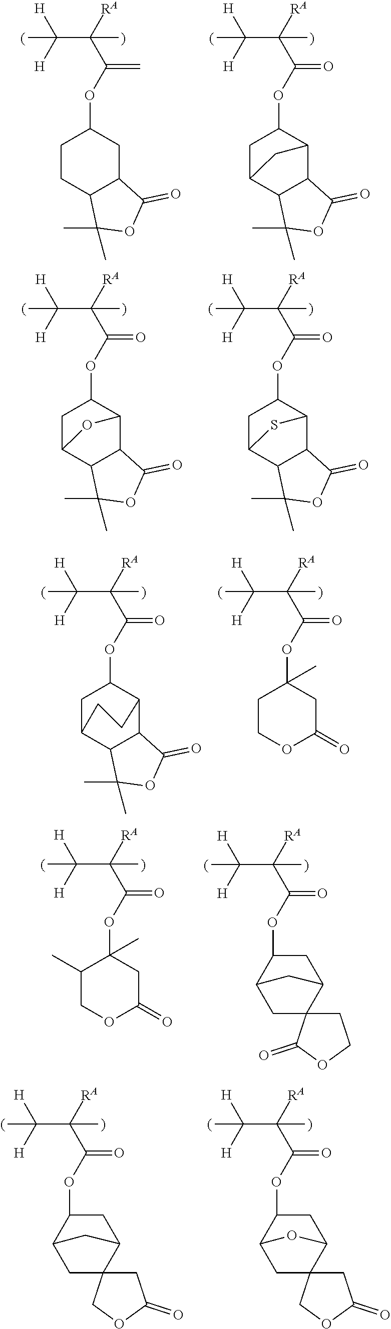

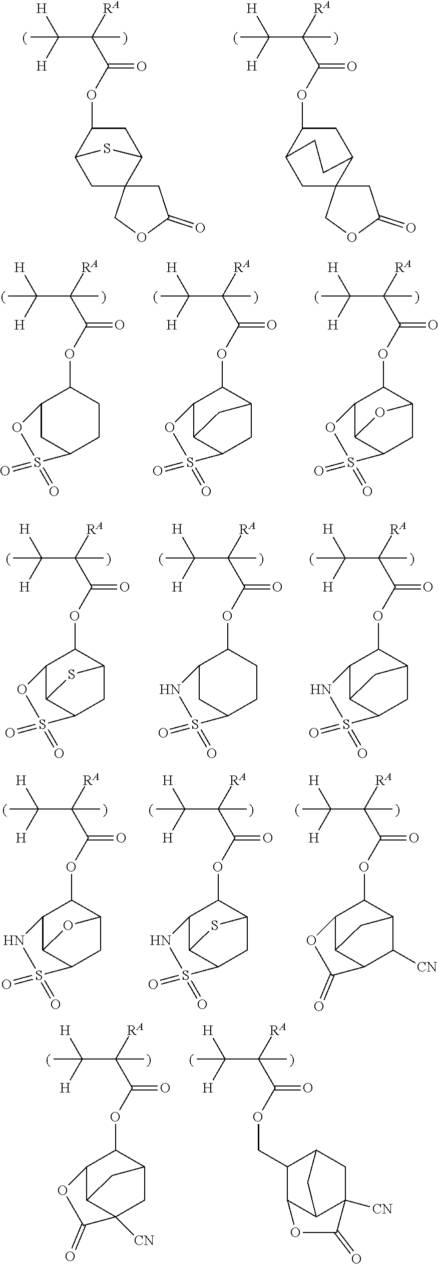

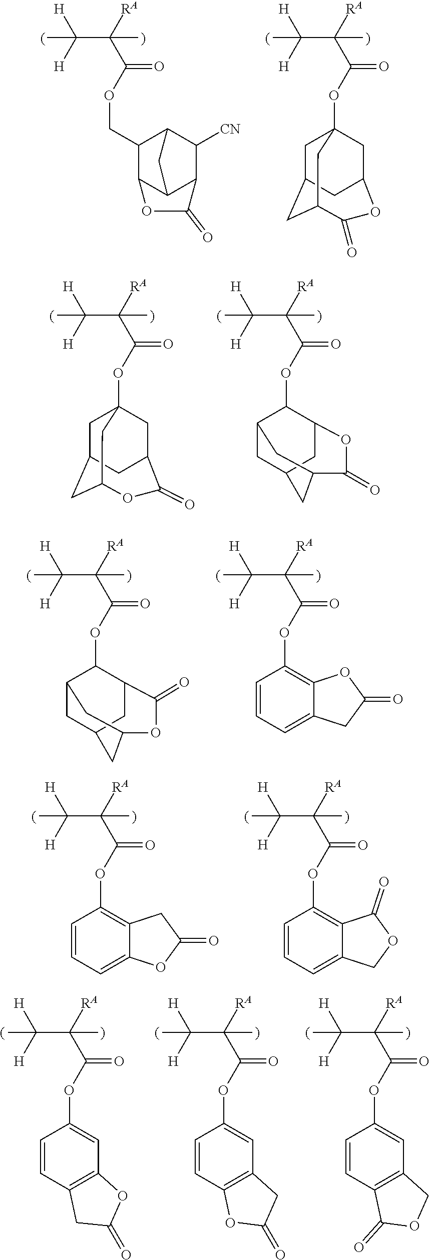

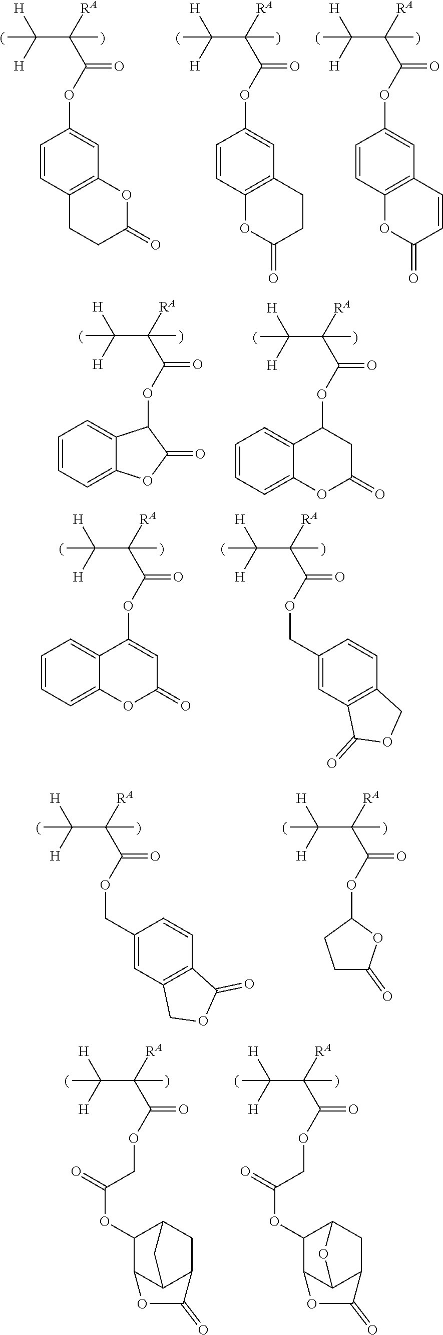

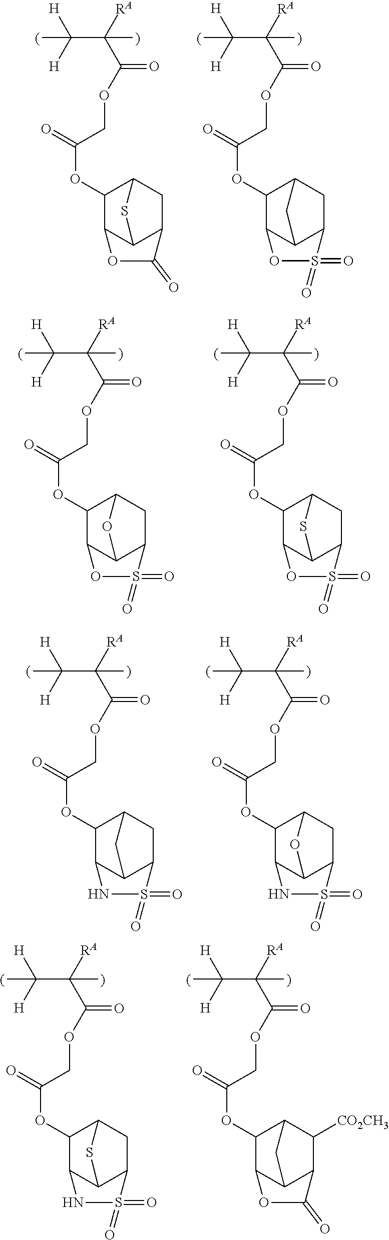

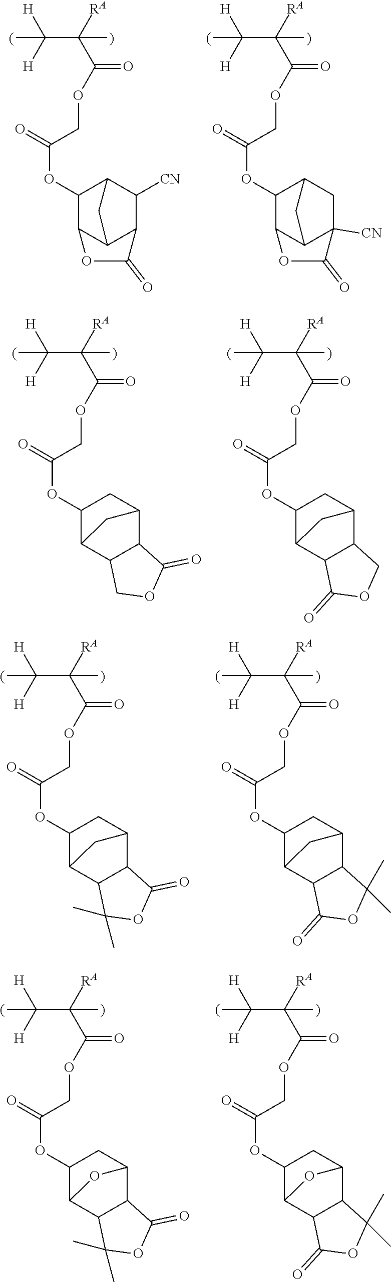

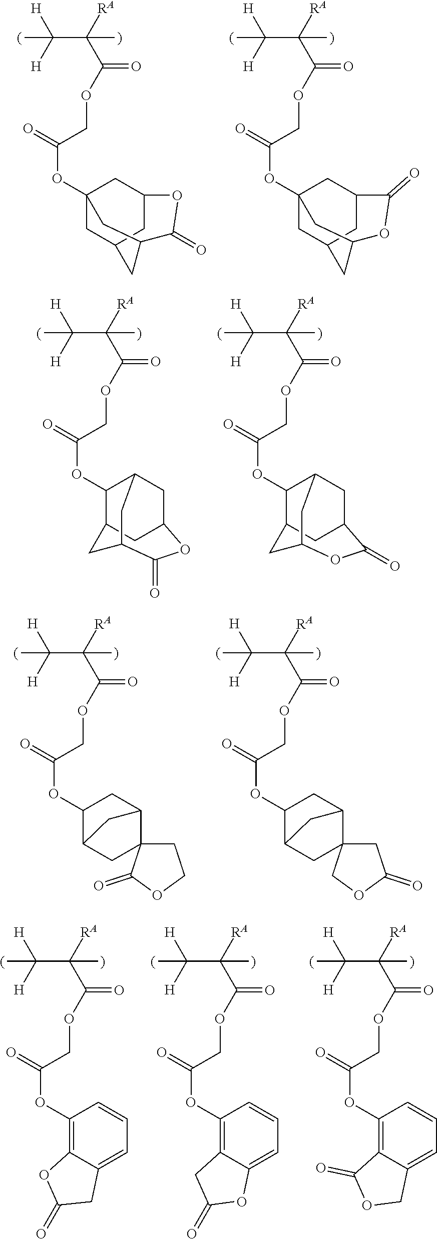

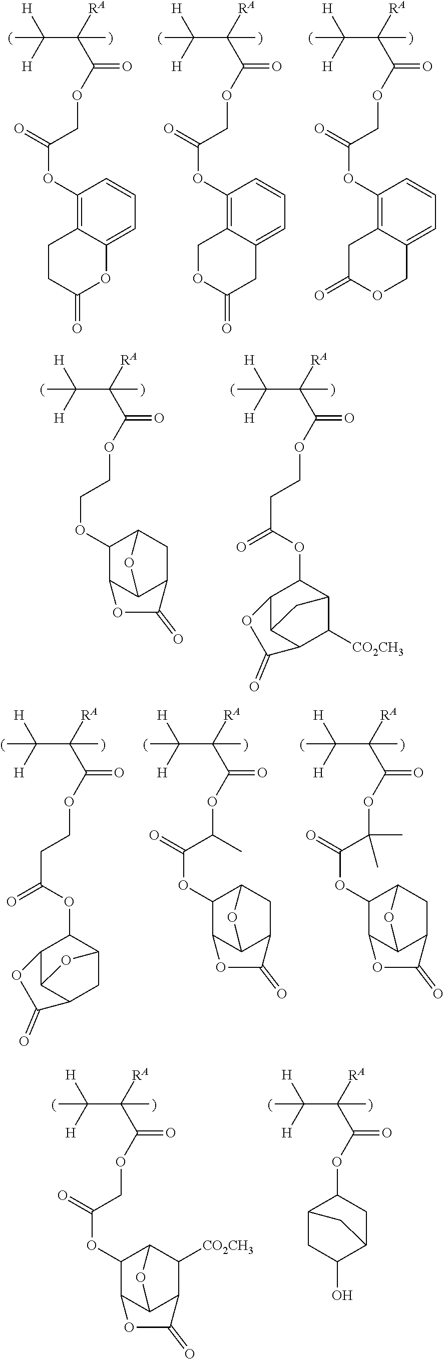

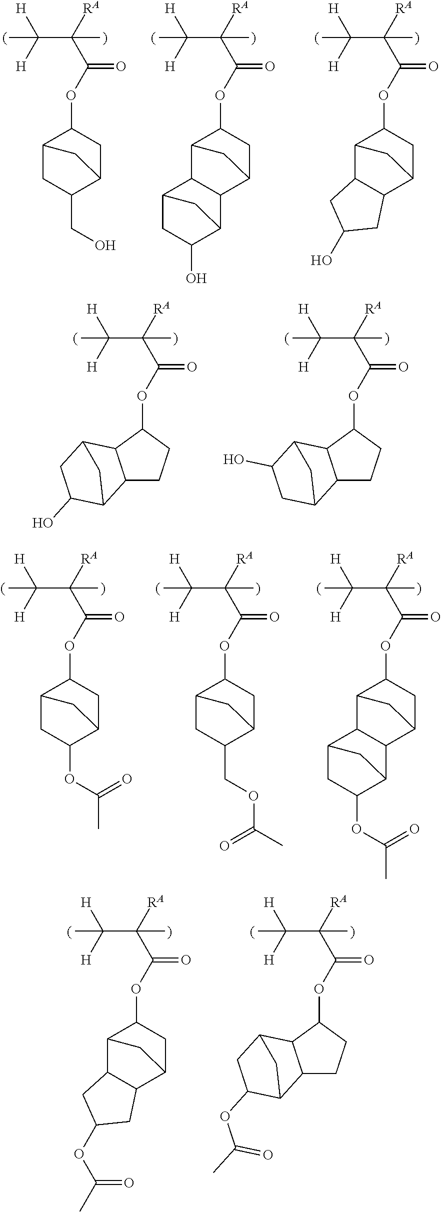

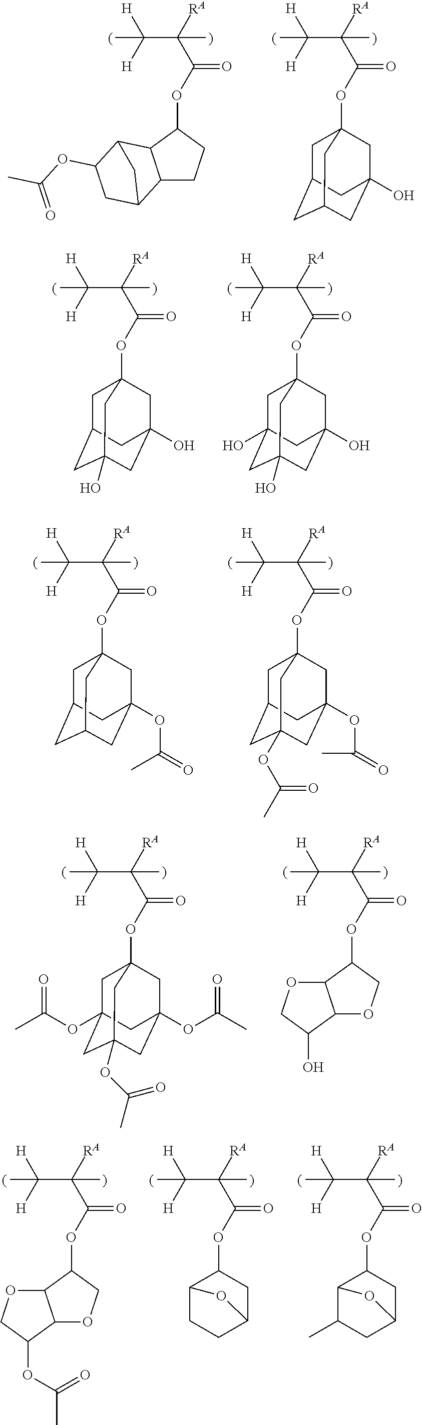

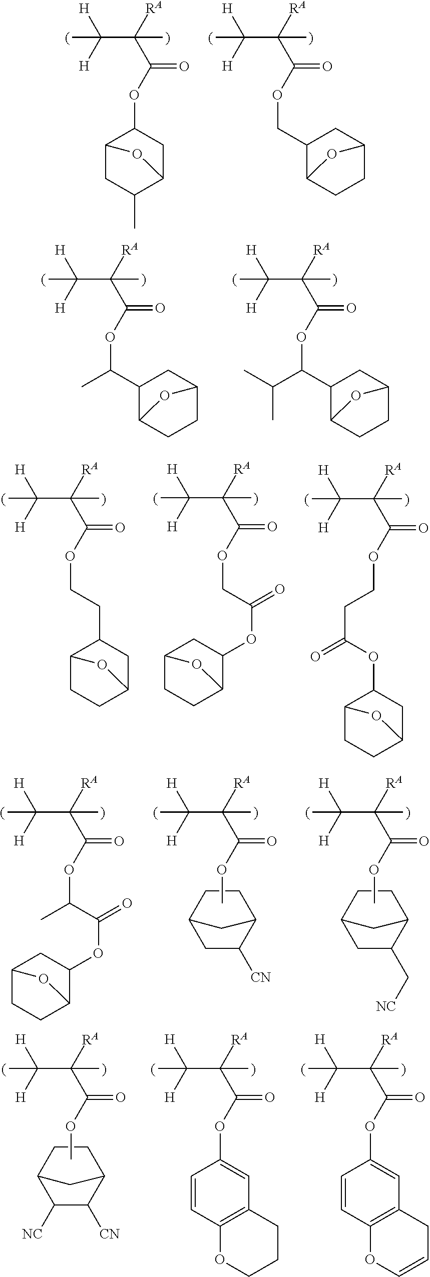

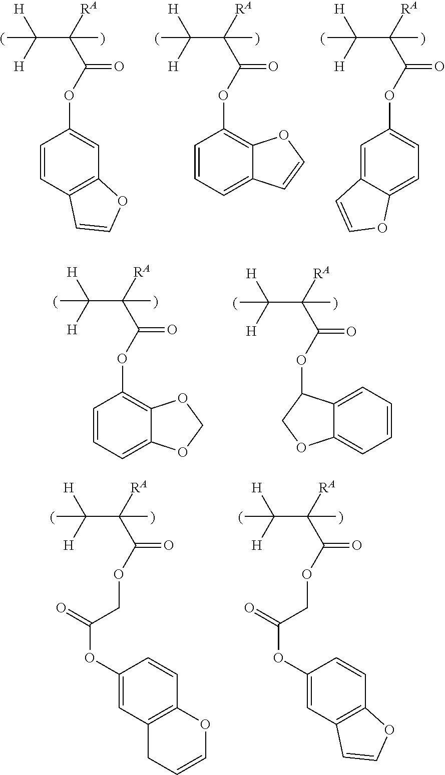

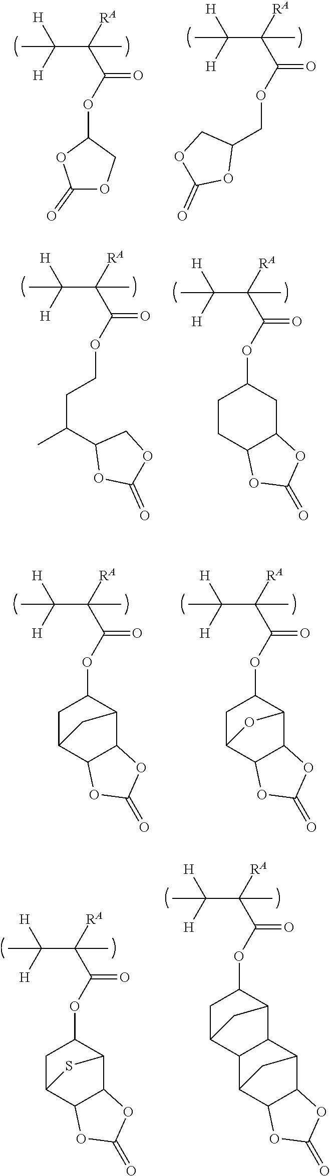

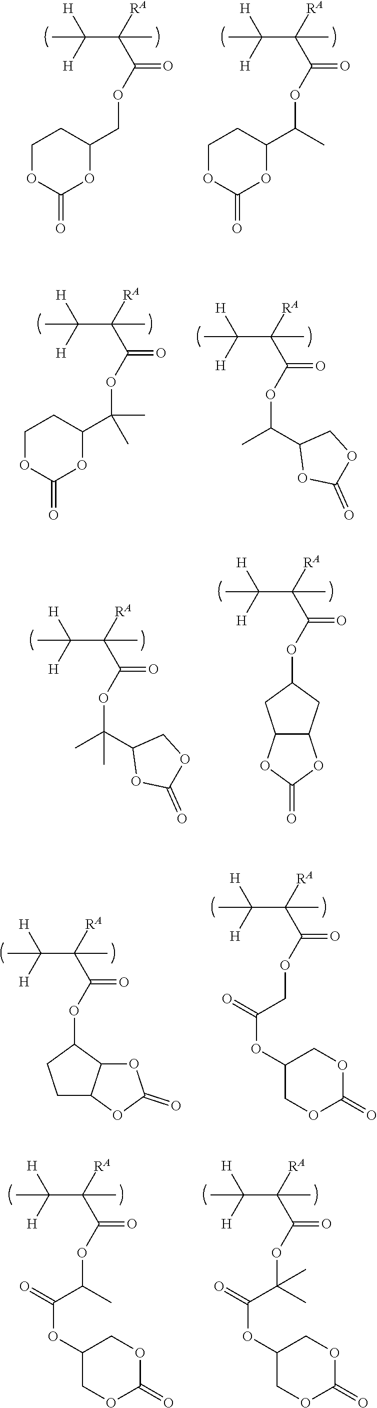

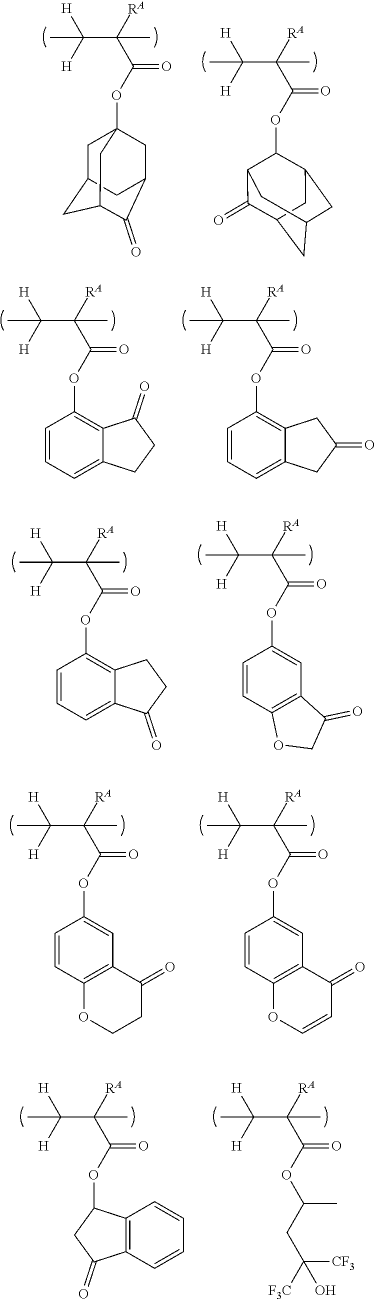

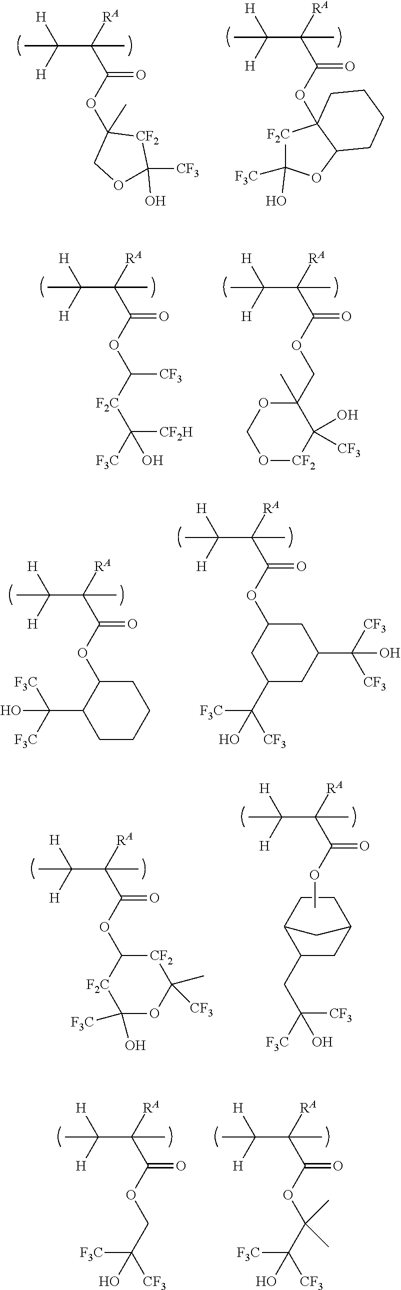

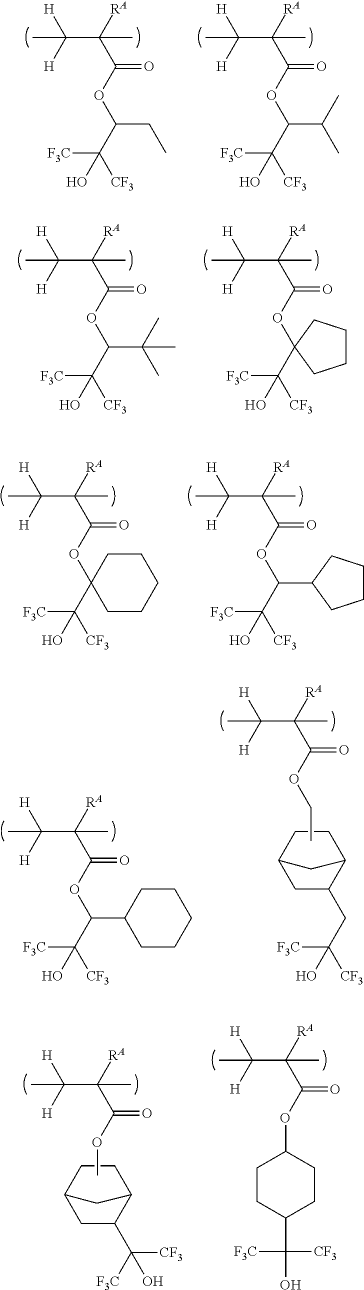

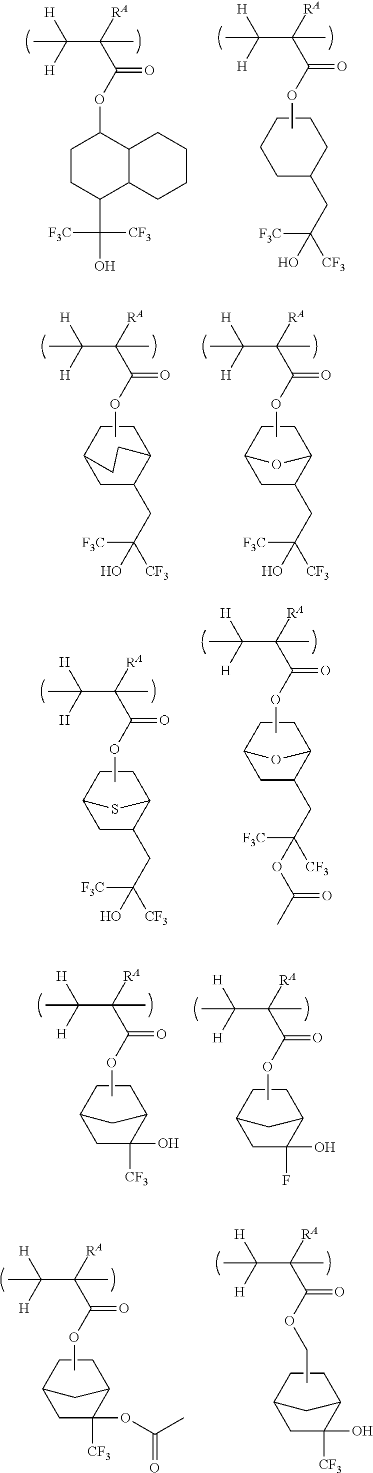

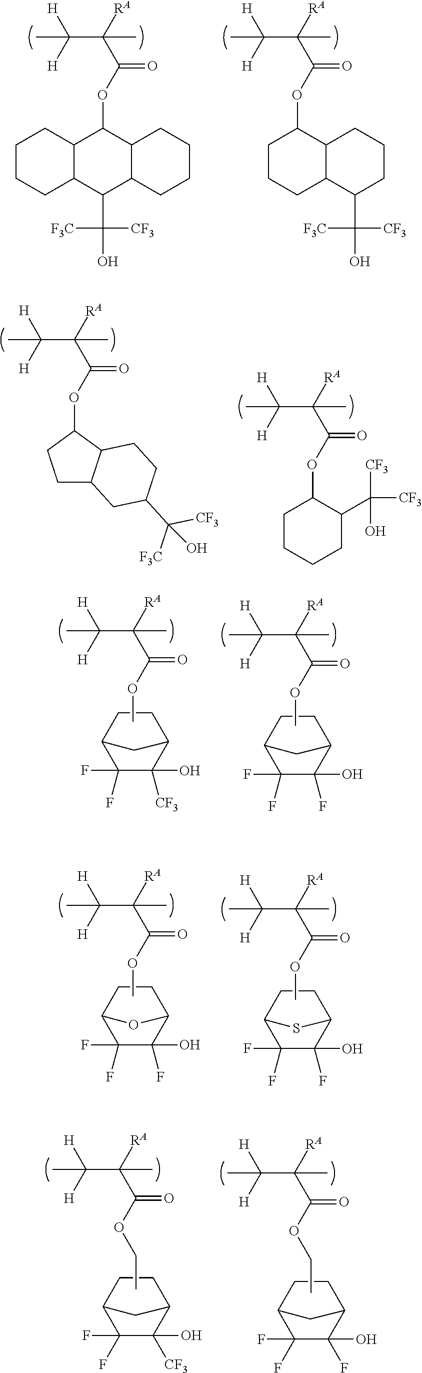

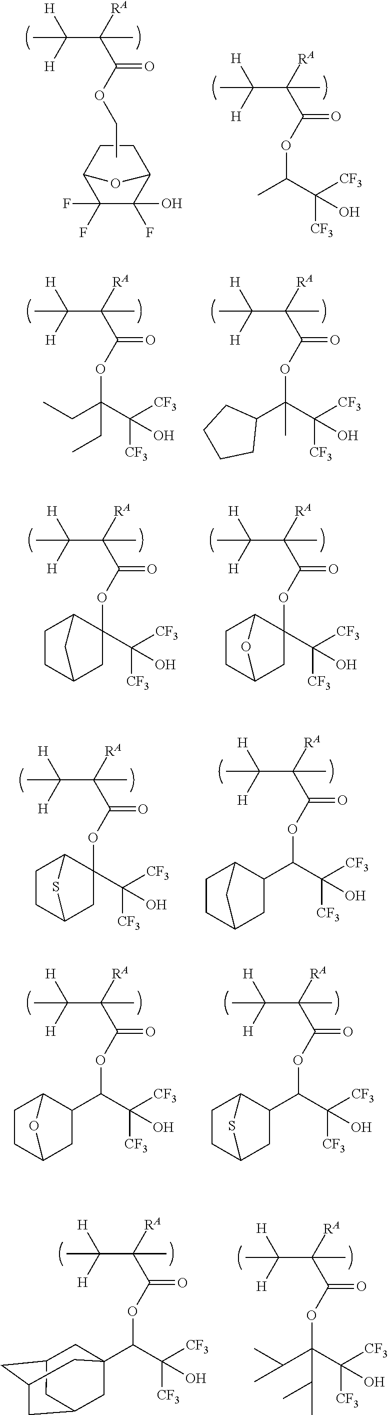

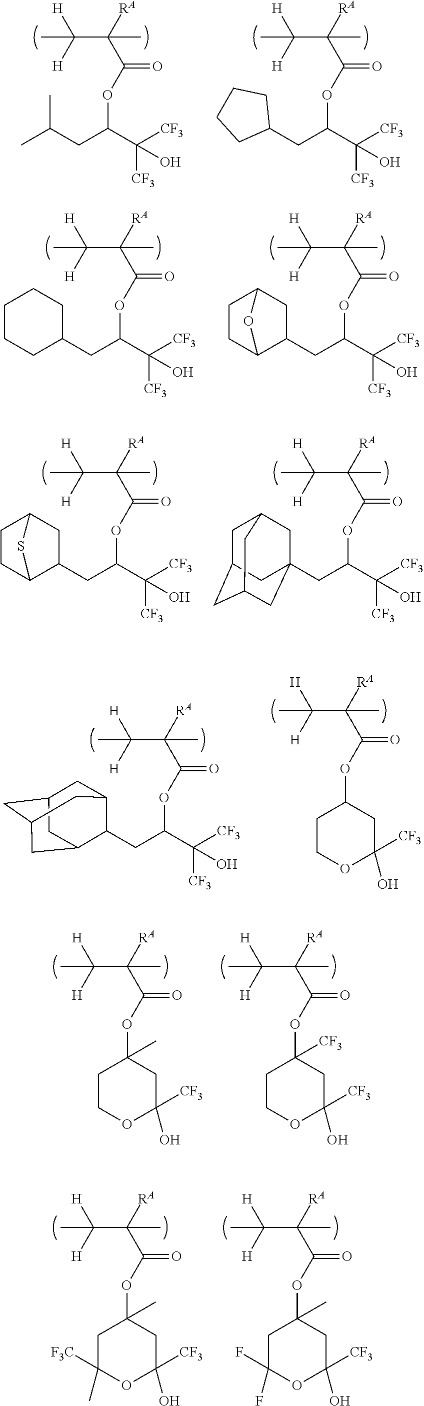

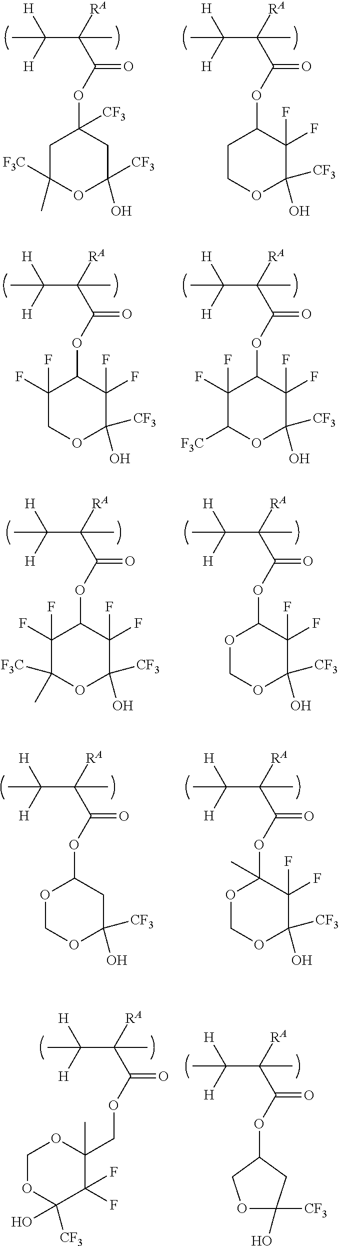

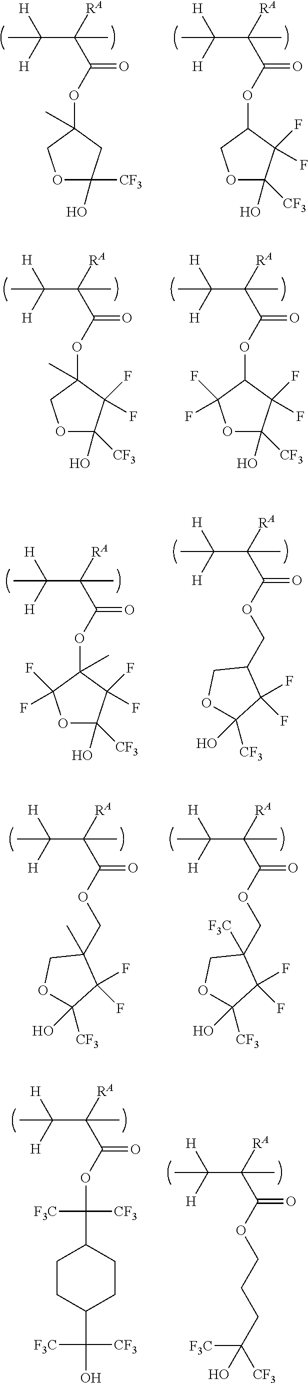

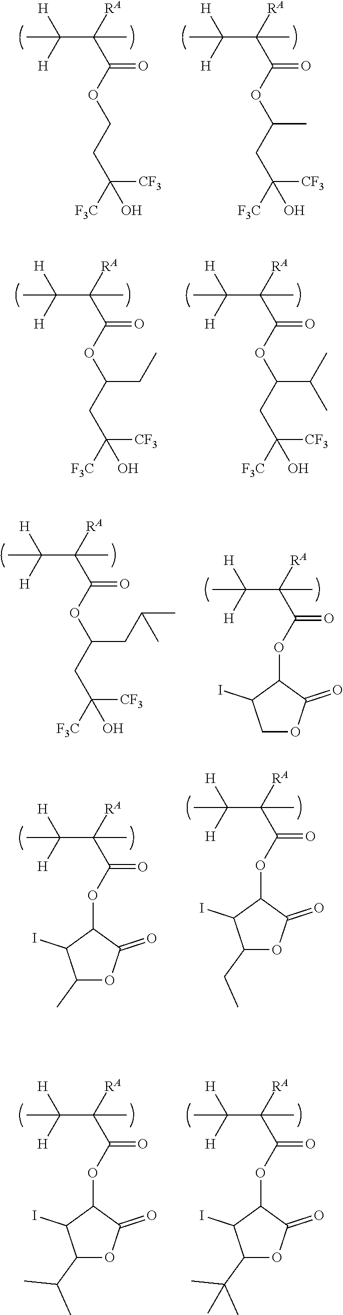

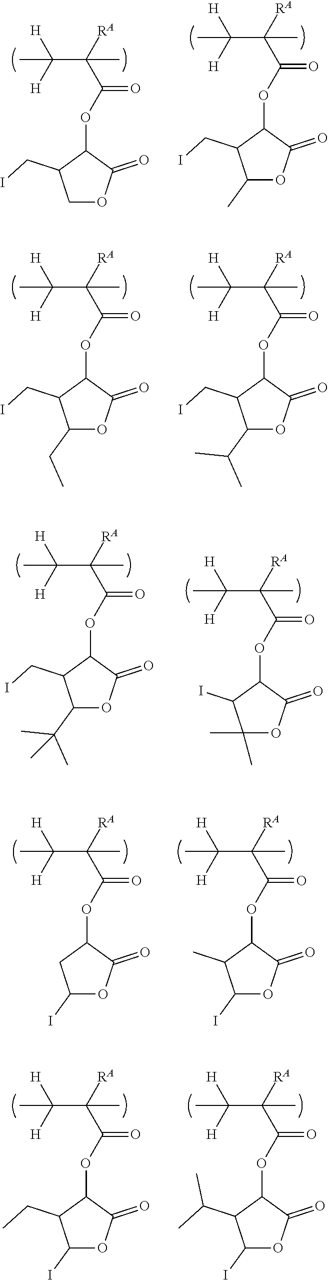

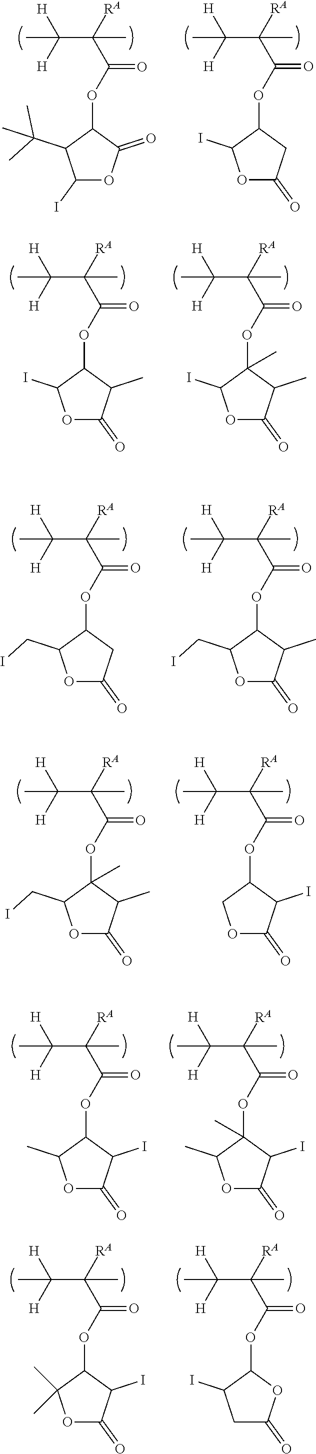

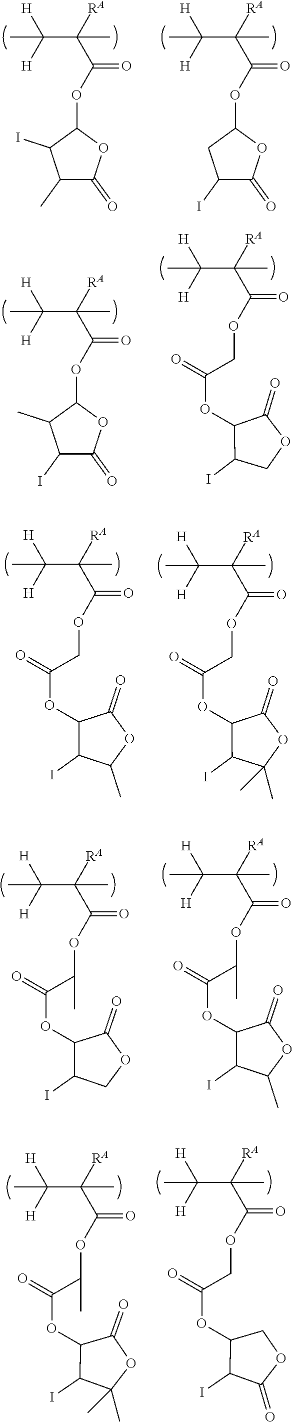

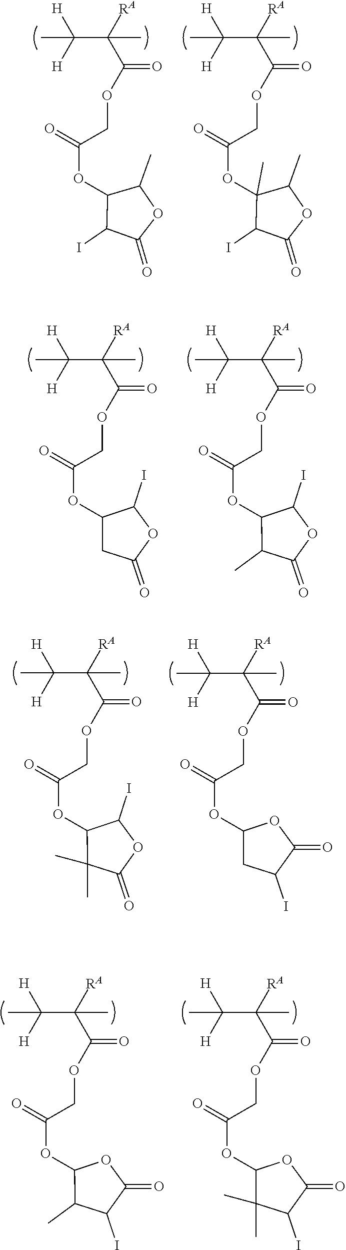

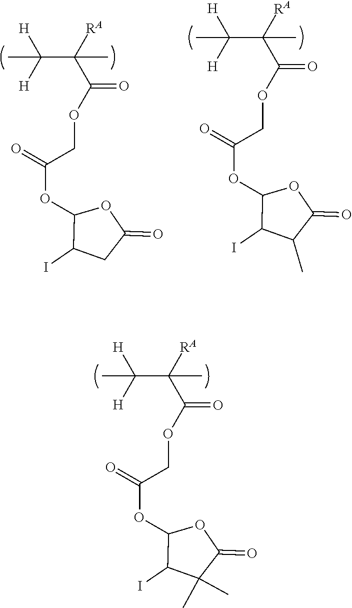

Specific examples of the repeat units b2 are shown below, but not limited thereto. In the following formula. R^{A} is as defined above.

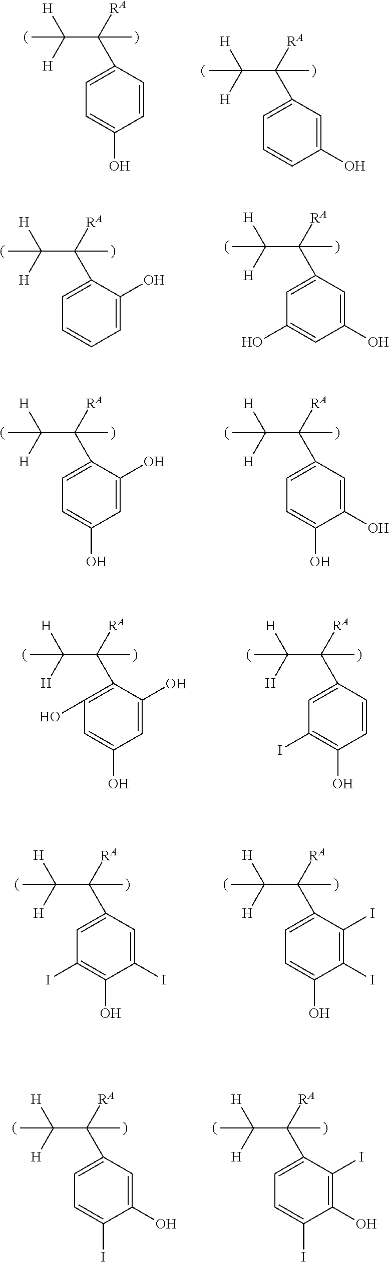

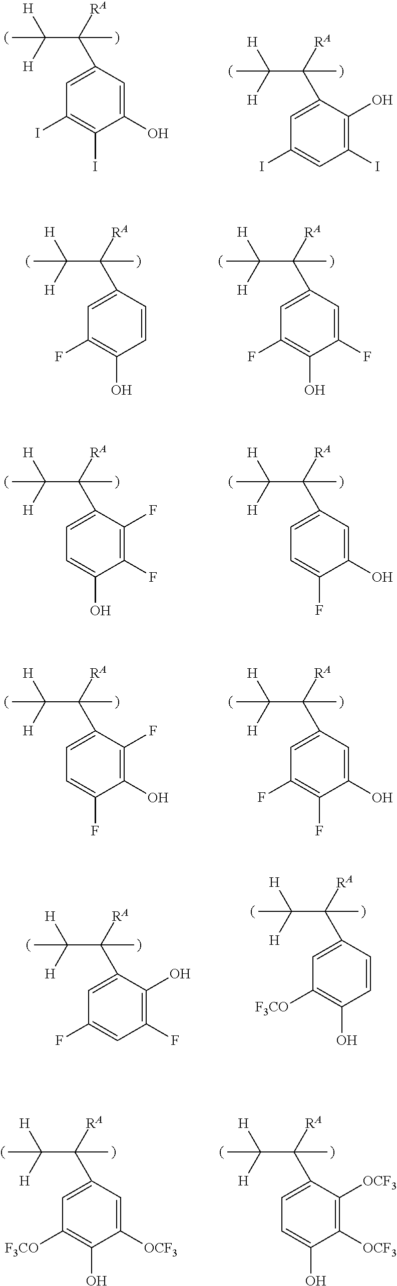

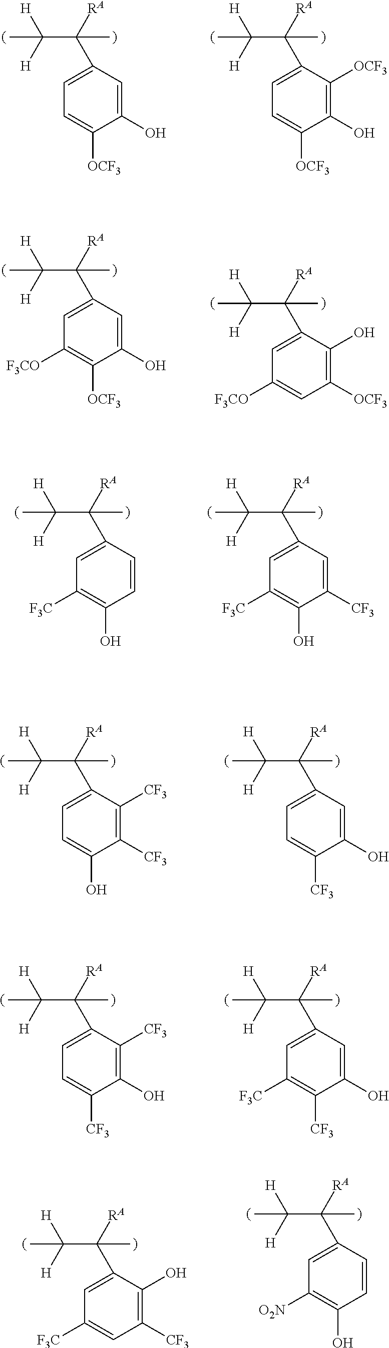

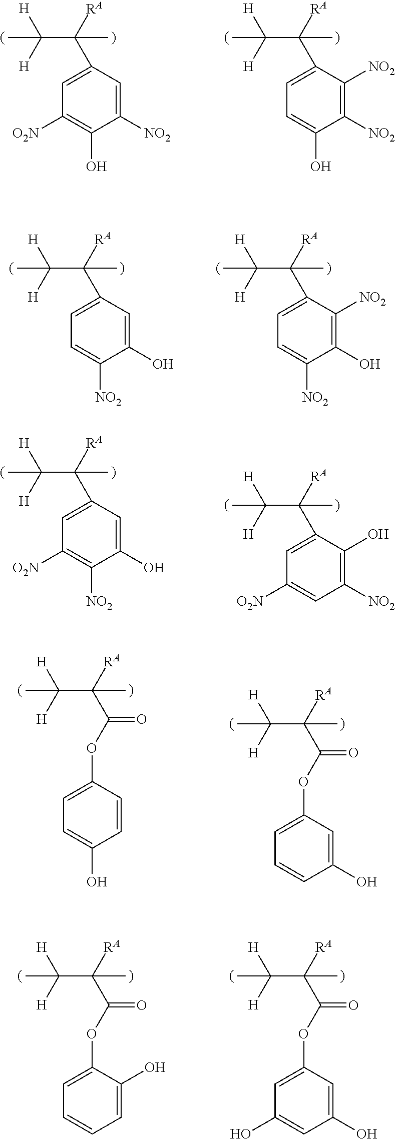

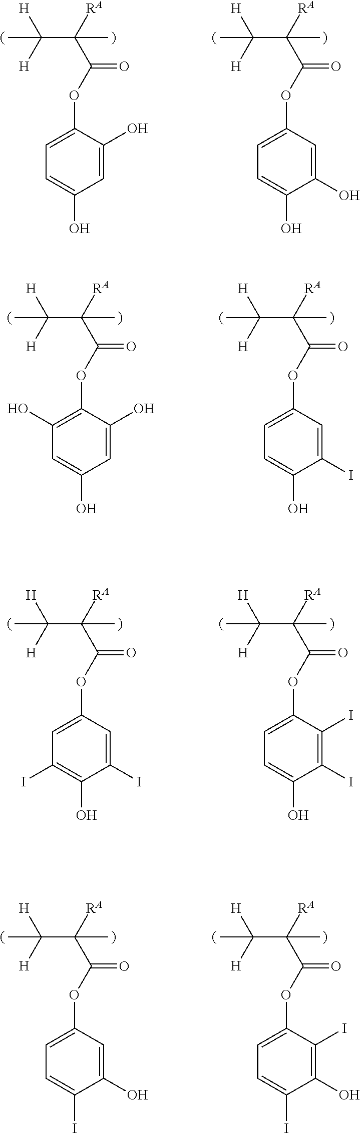

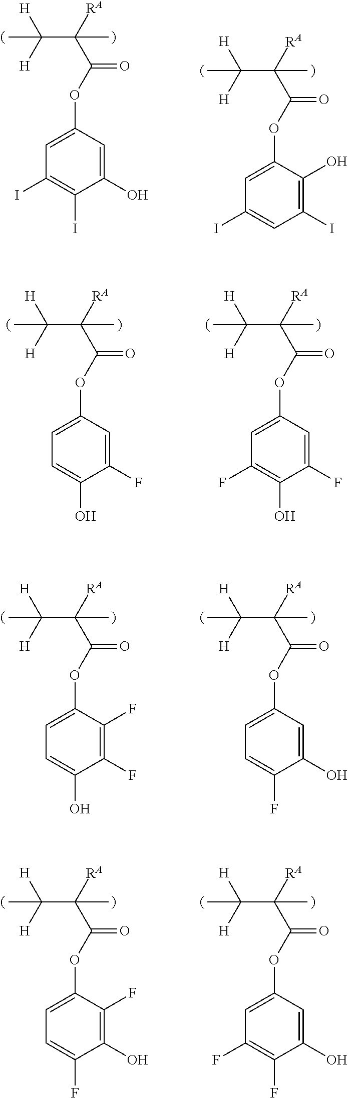

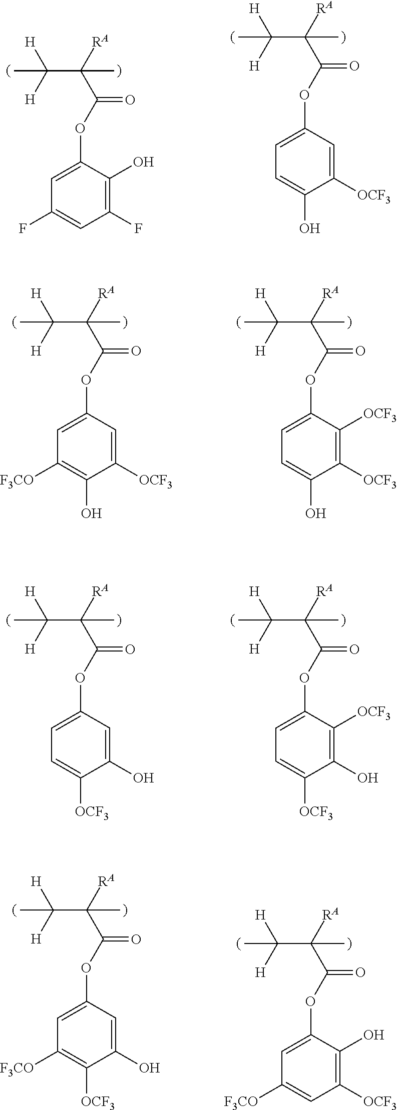

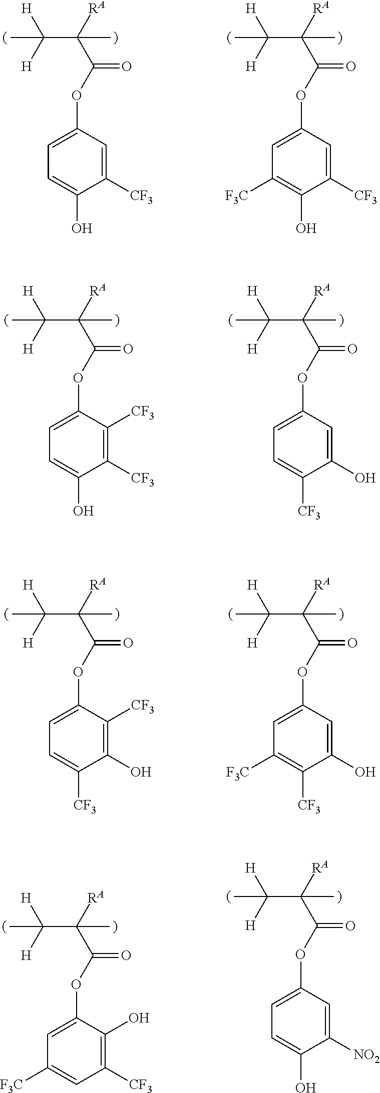

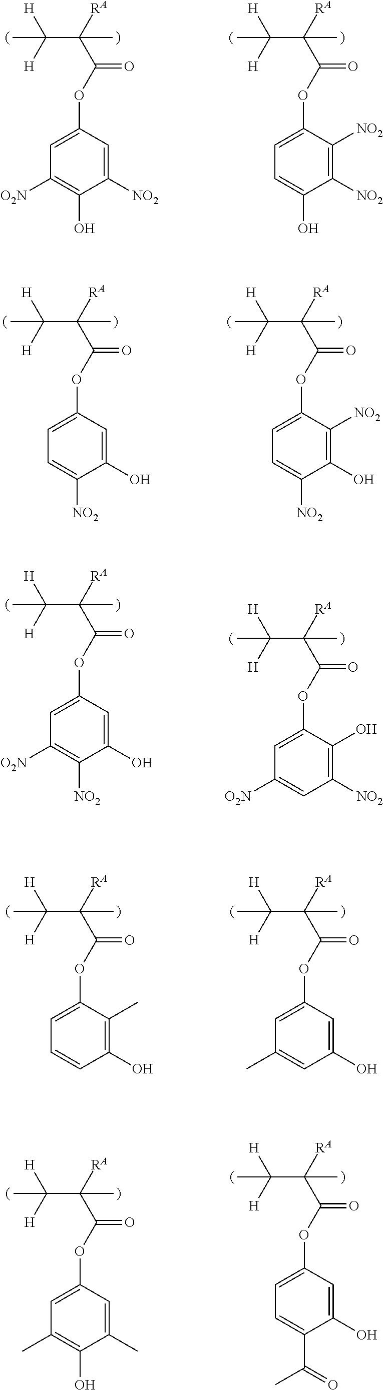

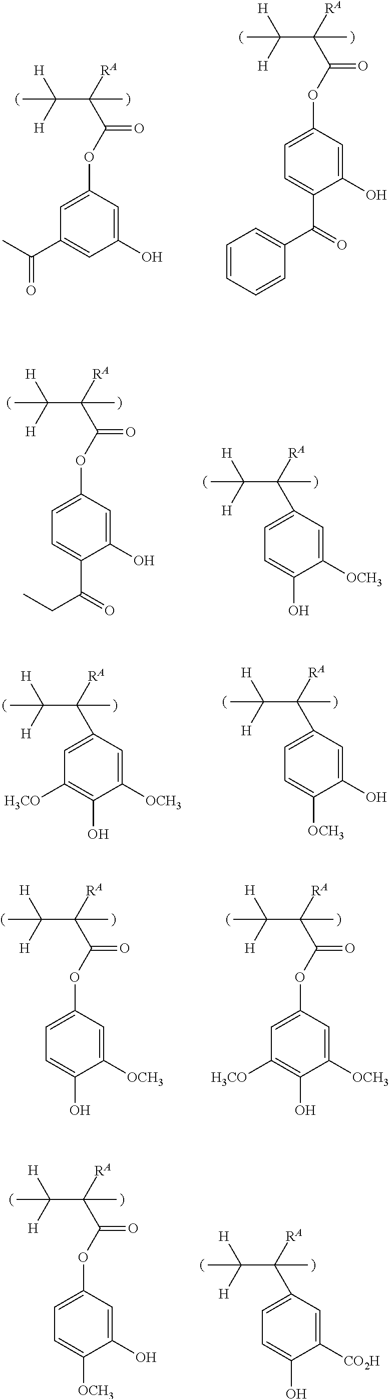

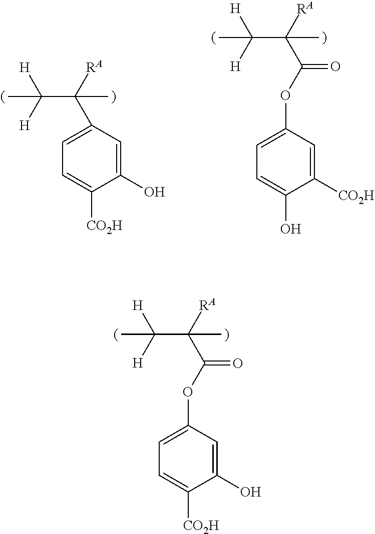

As the repeat units bl or b2, those units having a lactone ring as a polar group are particularly preferred in the case of ArF lithography, and those units having a phenol site as a polar group are preferred in the case of KrF lithography. EB lithography, and EUV lithography.

The base polymer may include repeat units having a structure having a hydroxy group protected with an acid labile group (also referred to as repeat units c, hereinafter). The repeat unit c is not particularly limited as long as the unit includes one or two or more structures having a hydroxy group protected with a protective group such that the protective group is decomposed to generate the hydroxy group under the action of acid, but repeat units having the following formula (c1) are preferable:

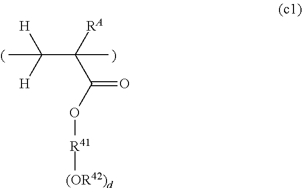

In formula (c1), R^{A} is a hydrogen atom, a fluorine atom, a methyl group, or a trifluoromethyl group. R^{q1} is a C₁-C₃₀ (d+1)-valent hydrocarbon group which may contain a heteroatom. R⁴² is an acid labile group. d is 1, 2, 3, or 4.

In formula (c1), the acid labile group represented by R⁴² may be any group that is deprotected under the action of acid so that a hydroxy group is generated. The structure of R⁴² is not particularly limited, an acetal structure, a ketal structure, an alkoxycarbonyl group, an alkoxymethyl group having the following formula (c2), and the like are preferable, and an alkoxymethyl group having the following formula (c2) is particularly preferable:

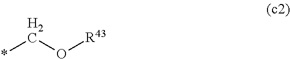

wherein * designates a point of attachment, and R⁴³ is a C₁-C₁₅ hydrocarbyl group.

Specific examples of the acid labile group represented by R⁴², the alkoxymethyl group having formula (c2), and the repeat units c are as exemplified for the repeat units c described in JP-A 2020-111564.

The base polymer may include repeat units d derived from indene, benzofuran, benzothiophene, acenaphthylene, chromone, coumarin, norbornadiene, or derivatives thereof. Specific examples of the monomer from which repeat units d are derived are shown below, but not limited thereto.

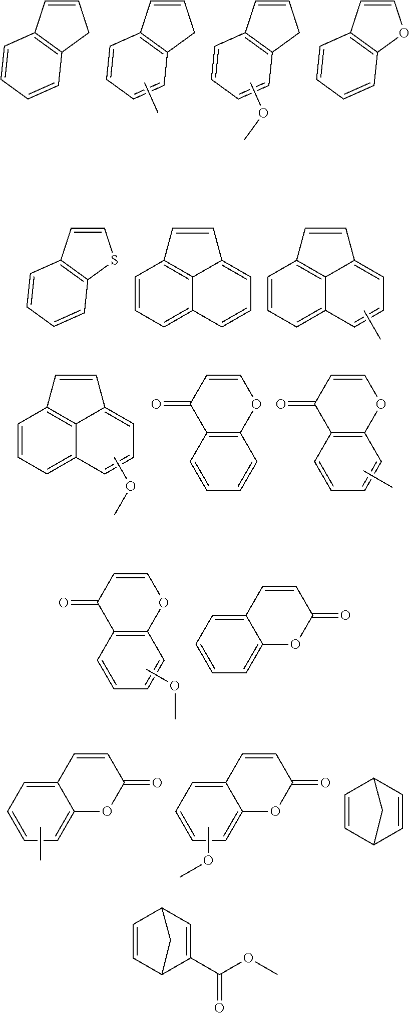

The base polymer may further include repeat units e derived from indane, vinylpyridine, or vinylcarbazole.

In the polymer of the present invention, the repeat units A, a1, a2, a3, b1, b2, c, d, and e are incorporated in a ratio of preferably 0<A≤0.4, 0≤a1≤0.8, 0≤a2≤0.8, 0≤a3≤0.6, 0<a1+a2+a3≤0.8, 0≤b1≤0.6, 0≤b2≤0.6, 0≤c≤0.5, 0≤d≤0.3, and 0≤e≤0.3, more preferably 0<A≤0.3, 0≤a1≤0.7, 0≤a2≤0.7, 0≤a3≤0.5, 0<a1+a2+a3≤0.7, 0≤b1≤0.5, 0≤b2≤0.5, 0≤c≤0.3, 0≤d≤0.3, and 0 Se≤0.3. Provided that A+a1+a2+a3+b1+b2+c+d+e≤1.0.

The weight average molecular weight (Mw) of the polymer is preferably 1000 to 500000 and more preferably 3000 to 100000. When Mw is in this range, sufficient etching resistance is obtained, and there is no possibility of degradation of resolution due to a failure to acquire a difference in dissolution rate before and after exposure. In the present invention, Mw is a value measured by gel permeation chromatography (GPC) with THF or N,N-dimethylformamide (DMF) as a solvent, and calculated as polystyrene.

Since the influence of the molecular weight distribution (Mw/Mn) becomes stronger as the pattern rule becomes finer, the Mw/Mn of the polymer preferably has narrow dispersity of 1.0 to 2.0 in order to obtain a resist composition suitable for micropatterning to a small feature size. Within the above range, there is little polymer having a low molecular weight or a high molecular weight, and there is no possibility that foreign matter is observed on the pattern or the shape of the pattern is deteriorated after exposure.

In order to synthesize the polymer, for example, a monomer from which the foregoing repeat units are derived may be heated in an organic solvent with a radical polymerization initiator added thereto to perform polymerization.

Specific examples of the organic solvent used during polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,l′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., more preferably 60 to 100° C. The reaction time is preferably 2 to 24 hours, more preferably 2 to 12 hours from the viewpoint of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form an ultra high-molecular-weight polymer, it is preferred from the viewpoint of quality control to prepare the monomer solution and the initiator solution separately and add them dropwise. The acid labile group that has been incorporated in the monomer may be kept as such, or polymerization may be followed by protection or partial protection. Any known chain transfer agent such as dodecyl mercaptan or 2-mercaptoethanol may be used in combination for molecular weight control purpose. In this case, these chain transfer agents are preferably added in an amount of 0.01 to 20 mol % based on the total of monomers to be polymerized.

In the case of a monomer containing a hydroxy group, the hydroxy group may be substituted with an acetal group susceptible to deprotection with an acid such as an ethoxyethoxy group during polymerization, and then deprotected by a weak acid and water, or may be substituted with an acetyl group, a formyl group, a pivaloyl group, or the like, and then alkaline hydrolysis may be performed after polymerization.

When hydroxystyrene or hydroxyvinylnaphthalene is copolymerized, hydroxystyrene or hydroxyvinylnaphthalene and another monomer may be heated and polymerized by adding a radical polymerization initiator in an organic solvent, but acetoxystyrene or acetoxyvinylnaphthalene may be used, and the acetoxy group may be deprotected by alkaline hydrolysis after polymerization to obtain polyhydroxystyrene or hydroxypolyvinylnaphthalene.

As a base during the alkaline hydrolysis, aqueous ammonia, triethylamine, or the like can be used. The reaction temperature is preferably-20 to 100° C., and more preferably 0 to 60° C. The reaction time is preferably 0.2 to 100 hours and more preferably 0.5 to 20 hours. The amount of each monomer in the monomer solution may be appropriately set, for example, so as to have a preferred content ratio of the repeat units.

Regarding the polymer obtained by the production method, a reaction solution resulting from polymerization reaction may be used as a final product, or a powder obtained through a purifying step such as re-precipitation method in which a polymerization liquid is added to a poor solvent to obtain a powder may be used as a final product, but from the viewpoints of operation efficiency and consistent quality, it is preferable to use a polymer solution obtained by dissolving the powder resulting from the purifying step in a solvent as a final product.

Specific examples of the solvent used at that time include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); high-boiling-point alcohol-based solvents such as diethylene glycol, propylene glycol, glycerin, 1,4-butanediol, and 1,3-butanediol; and a mixed solvent thereof, which are described in JP-A 2008-111103, paragraphs to [0145].

The polymer solution preferably has a polymer concentration of 0.01 to 30 wt %, more preferably 0.1 to 20 wt %.

The reaction solution or polymer solution is preferably filtered through a filter. Filtration is effective in terms of consistent quality because foreign matter and gel which may cause defects can be removed.

Examples of the material for the filter used for the filter filtration include fluorocarbon-based, cellulose-based, nylon-based, polyester-based, and hydrocarbon-based materials, and in the filtration step of the resist composition, a filter formed of a fluorocarbon-based material called Teflon®, a hydrocarbon-based material such as polyethylene and polypropylene, or nylon is preferable. While the pore size of the filter may be selected appropriate to comply with the desired cleanness, the filter preferably has a pore size of 100 nm or less, more preferably 20 nm or less. A single filter may be used or a plurality of filters may be used in combination. Although the filtering method may be single pass of the solution, more preferably the filtering step is repeated by flowing the solution in a circulating manner. In the polymer production step, the filtration step may be carried out any times, in any order and in any stage, but the reaction solution after the polymerization reaction or the polymer solution may be filtered, preferably both are filtered.

### Chemically Amplified Resist Composition

### Base Polymer (A)

The chemically amplified resist composition of the present invention comprises a base polymer containing the above-described polymer as the component (A).

The polymer may be used alone or in combination of two or more polymers which are different in compositional ratio, Mw and/or Mw/Mn. The base polymer (A) may contain a hydrogenated ring-opened metathesis polymer in addition to the polymer, and as the hydrogenated ring-opened metathesis polymer, a polymer described in JP-A 2003-66612 can be used.

### Organic Solvent (B)

The chemically amplified resist composition of the present invention may comprise an organic solvent as a component (B). The organic solvent (B) is not particularly limited as long as each component described above and each component described below can be dissolved. Specific examples of such an organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; ketoalcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL, and mixed solvents thereof.

Of the foregoing organic solvents, it is recommended to use 1-ethoxy-2-propanol, PGMEA, cyclohexanone, GBL, ethyl lactate, DAA, and mixed solvents thereof because the base polymer of the component (A) is most soluble therein.

The content of the organic solvent (B) in the chemically amplified resist composition of the present invention is preferably 200 to 7000 parts by weight and more preferably 400 to 5000 parts by weight per 80 parts by weight of the base polymer (A). The organic solvent (B) may be used alone or in admixture of two or more kinds thereof.

### Quencher (C)

The chemically amplified resist composition of the present invention may comprise a quencher as a component (C). In the present invention, the quencher is a material capable of trapping the acid generated by the photoacid generator in the chemically amplified resist composition to prevent the acid from diffusing to the unexposed area, for thereby forming a desired pattern.

Specific examples of the quencher (C) include an onium salt having the following formula (1) or (2).

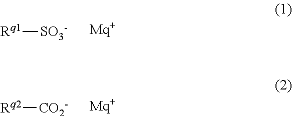

In formula (1), Ral is a hydrogen atom or a C₁-C₄₀ hydrocarbyl group which may contain a heteroatom, exclusive of the hydrocarbyl group in which the hydrogen atom bonded to the carbon atom at a-position of the sulfo group is substituted with a fluorine atom or a fluoroalkyl group. In formula (2), R⁴² is a hydrogen atom or a C₁-C₄₀ hydrocarbyl group which may contain a heteroatom.

Specific examples of the C₁-C₄₀ hydrocarbyl group represented by R^{q1} include C₁-C₄₀ alkyl groups such as a methyl group, an ethyl group, a n-propyl group, a sec-propyl group, a n-butyl group, a sec-butyl group, an isobutyl group, a tert-butyl group, a n-pentyl group, a tert-pentyl group, a n-hexyl group, a n-octyl group, a 2-ethylhexyl group, a n-nonyl group, and a n-decyl group; C₃-C₄₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.02.6] decyl group, and an adamantyl group; and C₆-C₄₀ aryl groups such as a phenyl group, a naphthyl group, and an anthracenyl group. Some or all of the hydrogen atoms in the hydrocarbyl group may be substituted with a group containing a heteroatom such as an oxygen atom, a sulfur atom, a nitrogen atom, or a halogen atom, some of —CH₂— in the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

Specific examples of the hydrocarbyl group represented by R^{q2} include, in addition to the substituents exemplified as specific examples of R^{q1}, fluorinated saturated hydrocarbyl groups such as a trifluoromethyl group and a trifluoroethyl group, and fluorinated aryl groups such as a pentafluorophenyl group and a 4-trifluoromethylphenyl group.

Specific examples of the anion of the onium salt having formula (1) are shown below. but not limited thereto.

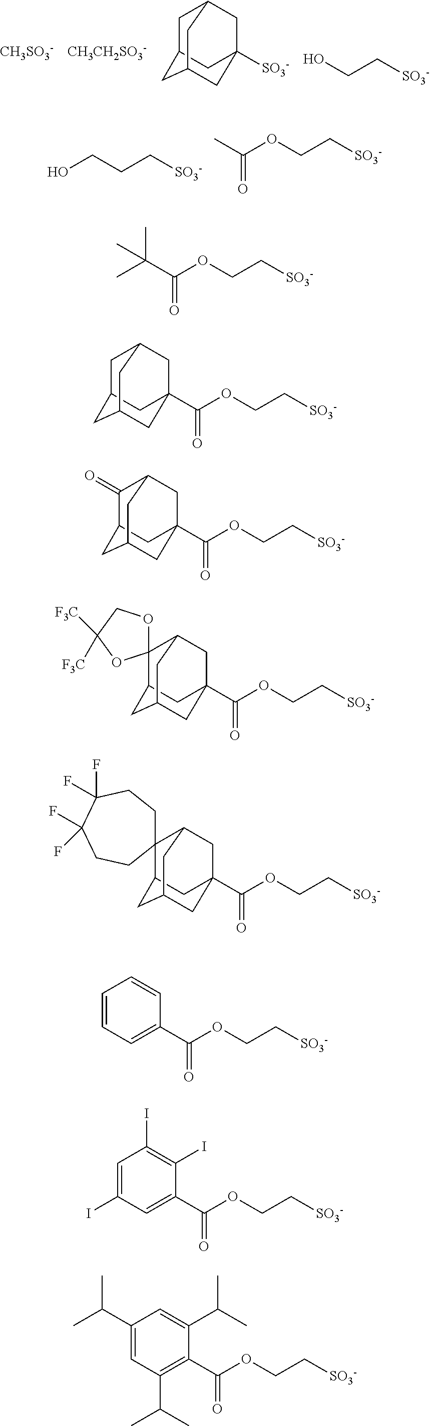

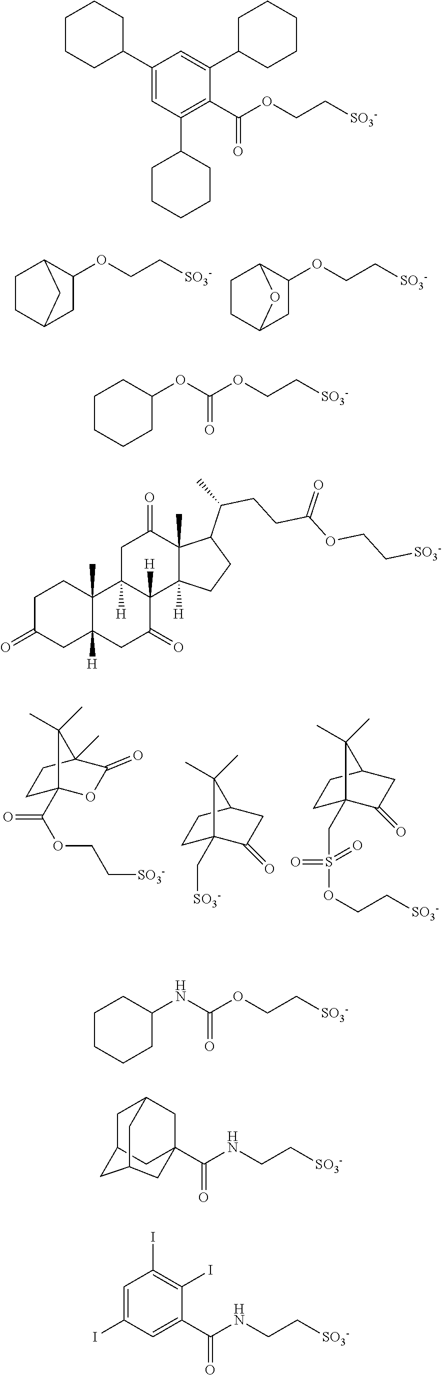

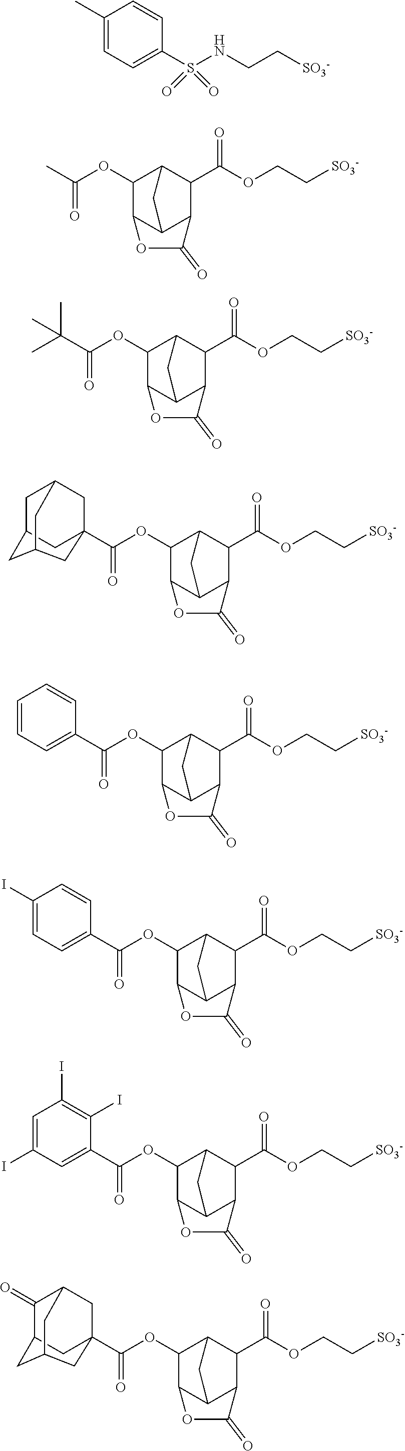

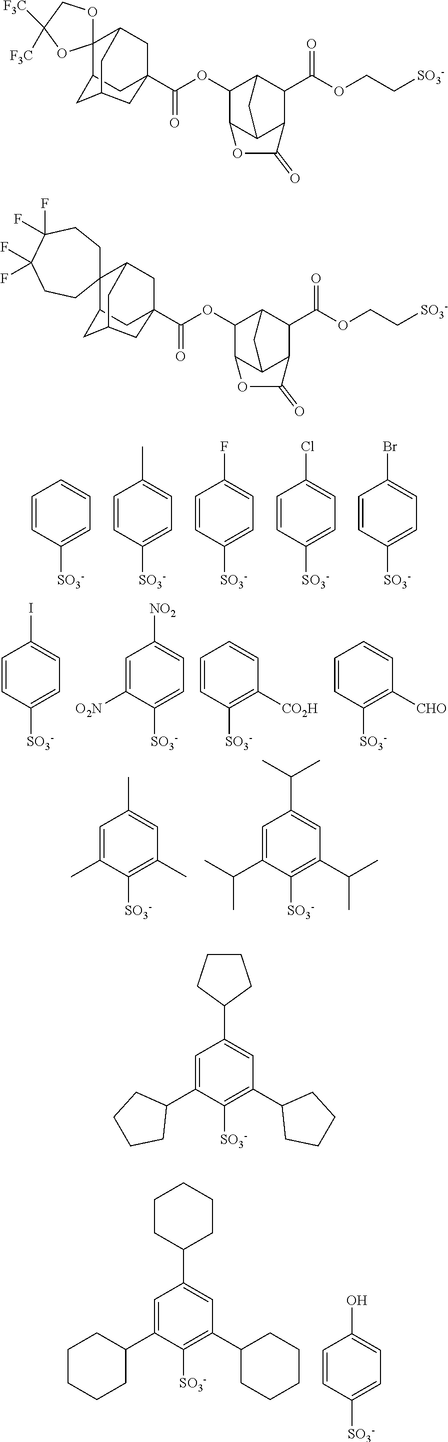

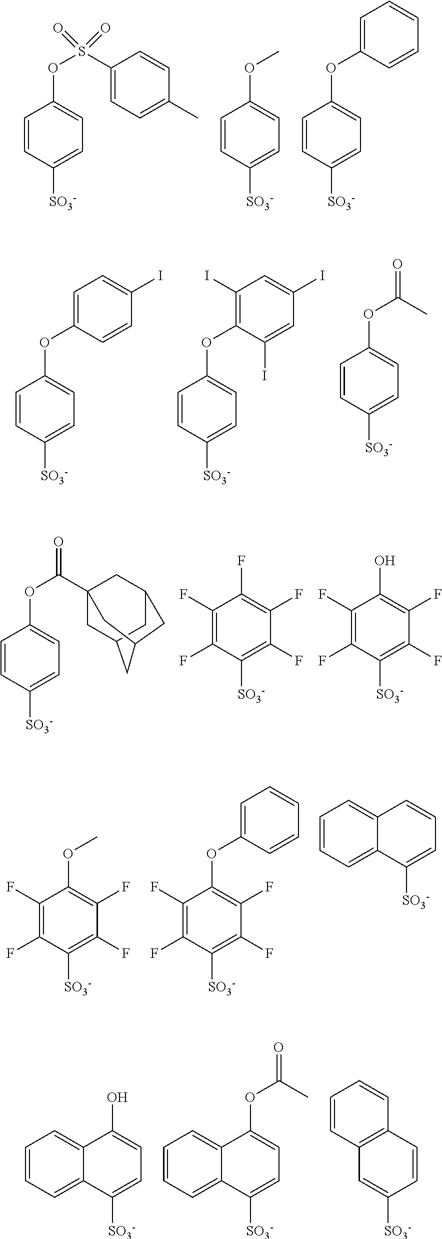

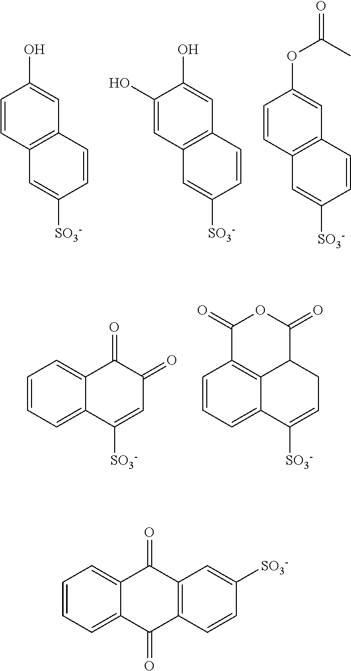

Specific examples of the anion of the onium salt having formula (2) are shown below. but not limited thereto.

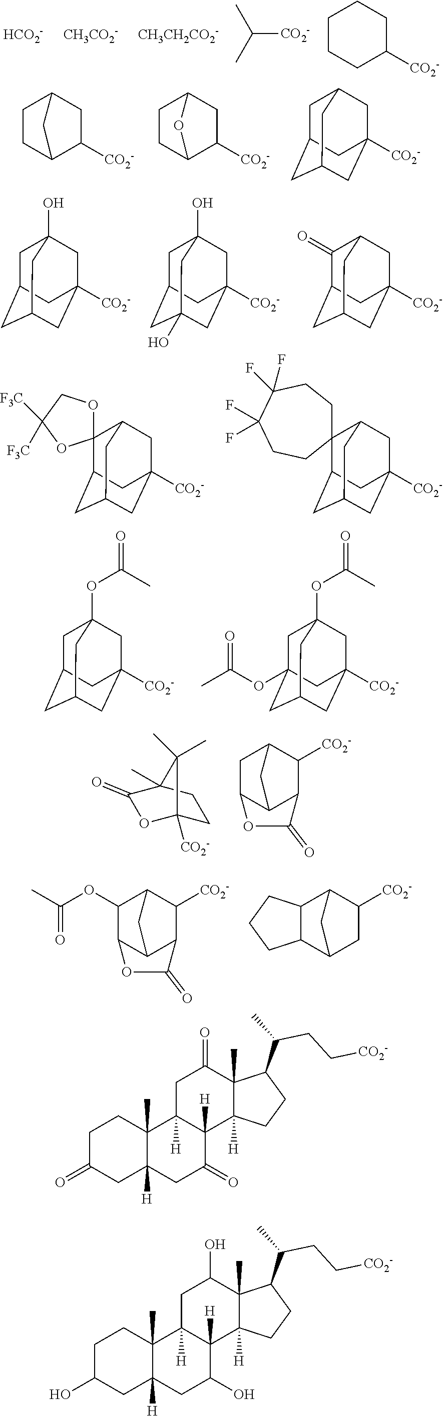

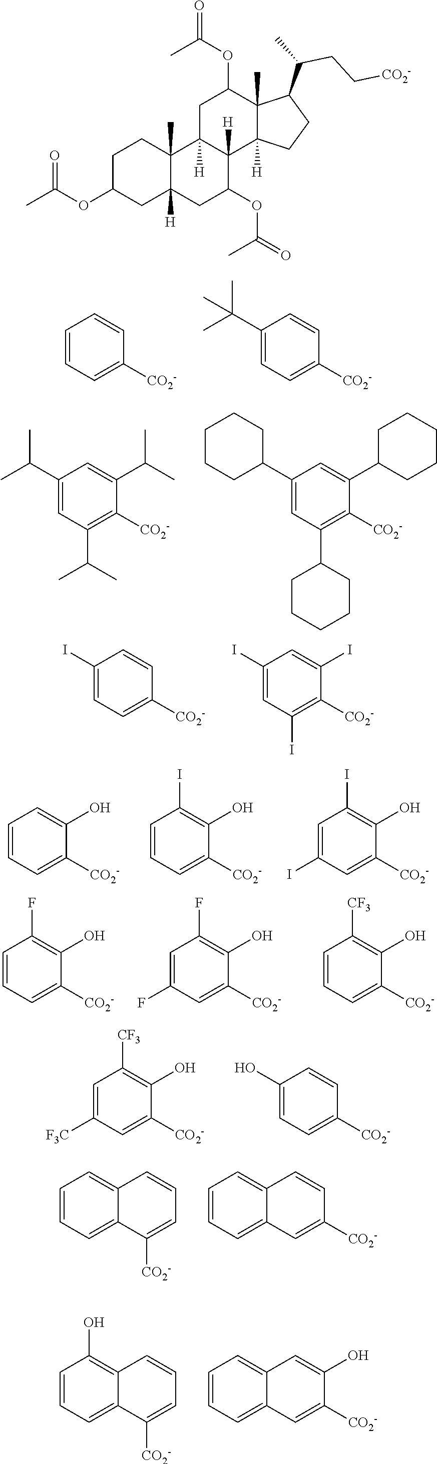

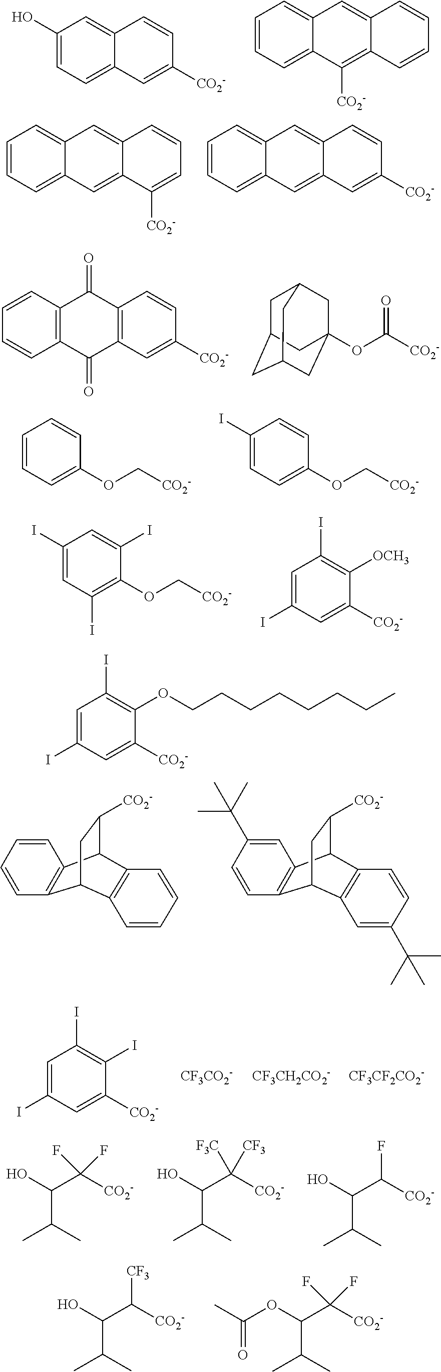

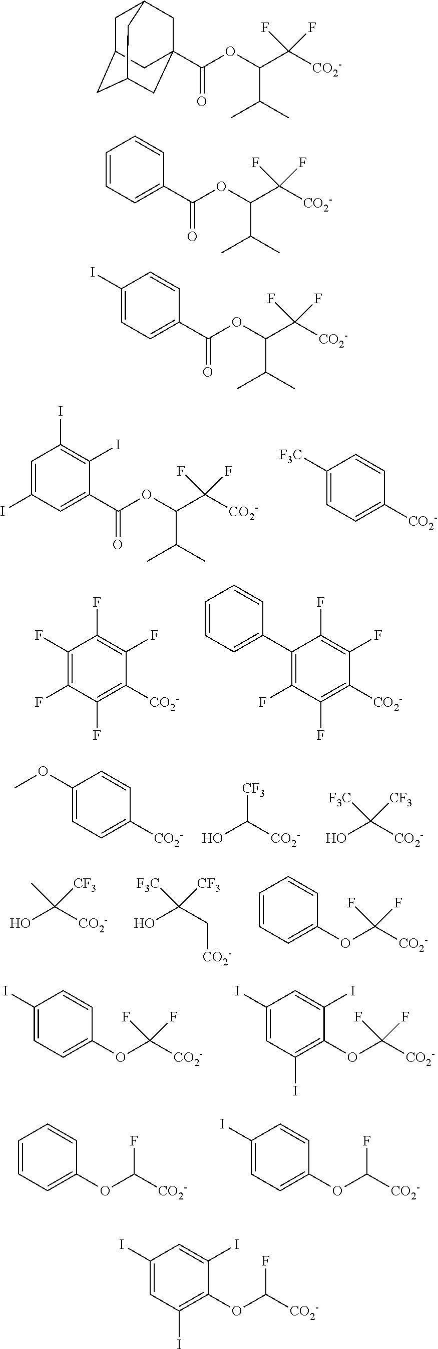

In formulae (1) and (2), Mq+is an onium cation. Examples of the onium cation include a sulfonium cation, an iodonium cation, and an ammonium cation. Specific examples of the sulfonium cation include those exemplified as specific examples of the sulfonium cation in formula (A), those described in paragraphs to of JP-A 2024-3744, those described in paragraphs to of WO 2024/128017, and those described in paragraphs to of JP 7491173, but not limited thereto.

The sulfonium cation is also preferably a sulfonium cation having the following formula (sulfo-1-1):

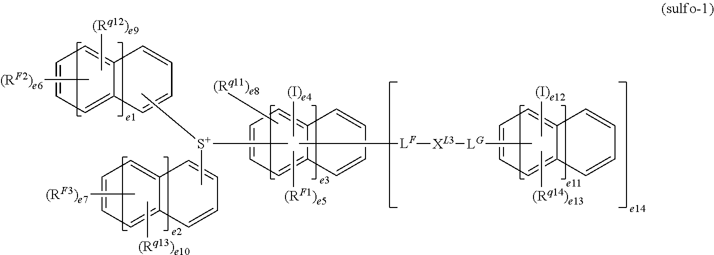

In formula (sulfo-1), e1 is 0 or 1. When e1 is 0, the relevant structure is a benzene ring, and when e1 is 1, the relevant structure is a naphthalene ring, but from the viewpoint of solvent solubility, the relevant structure is preferably a benzene ring in which e1 is 0. e2 is 0 or 1. When e2 is 0, the relevant structure is a benzene ring, and when e2 is 1, the relevant structure is a naphthalene ring, but from the viewpoint of solvent solubility, the relevant structure is preferably a benzene ring in which e2 is 0. e3 is 0 or 1. When e3 is 0, the relevant structure is a benzene ring, and when e3 is 1, the relevant structure is a naphthalene ring, but from the viewpoint of solvent solubility, the relevant structure is preferably a benzene ring in which e3 is 0.

In formula (sulfo-1), e4 is 0, 1, 2, 3, or 4. The larger the number of iodine atoms in the cationic structure is, the more the absorption for EUV is particularly enhanced, but since there is a concern that the solvent solubility becomes poor and precipitation occurs in the resist composition, e4 is preferably 0, 1, 2, or 3 and more preferably 0, 1, or 2.

In formula (sulfo-1), e5 is 0, 1, 2, 3, or 4. From the viewpoint of raw material availability, e5 is preferably 0, 1, 2, or 3 and more preferably 0, 1, or 2. e6 is 0, 1, 2, 3, 4, 5, or 6. From the viewpoint of raw material availability, e6 is preferably 0, 1, 2, or 3 and more preferably 0, 1, or 2. e7 is 0, 1, 2, 3, 4, 5, or 6. From the viewpoint of raw material availability, e7 is preferably 0, 1, 2, or 3 and more preferably 0, 1, or 2.

In formula (sulfo-1), e8 is 0, 1, or 2. From the viewpoint of raw material availability, e8 is preferably 0 or 1. e9 is 0, 1, or 2. From the viewpoint of raw material availability, e9 is preferably 0 or 1. e10 is 0, 1, or 2. From the viewpoint of raw material availability, e10 is preferably 0 or 1.

In formula (sulfo-1), e11 is 0 or 1. When e11 is 0, the relevant structure is a benzene ring, and when e11 is 1, the relevant structure is a naphthalene ring, but from the viewpoint of solvent solubility, the relevant structure is preferably a benzene ring in which e11 is 0.

In formula (sulfo-1), e12 is 0, 1, 2, 3, or 4. The larger the number of iodine atoms in the cationic structure is, the more the absorption for EUV is particularly enhanced, but since there is a concern that the solvent solubility becomes poor and precipitation occurs in the resist composition, e12 is preferably 0, 1, 2, or 3 and more preferably 0, 1, or 2.

In formula (sulfo-1), e13 is 0, 1, or 2. From the viewpoint of raw material availability, e13 is preferably 0 or 1. e14 is 0, 1, or 2. From the viewpoint of synthesis, e14 is preferably 0 or 1.

Provided that, when e1 is 0, 0≤e6+e9≤4, and when e1 is 1, 0≤e6+e9≤6. When e2 is 0, 0≤e7+e10≤4, and when e2 is 1, 0≤e7+e10≤6. When e3 is 0, 1≤e4+e5+e8+e14≤4, and when e3 is 1, 1≤e4+e5+e8+e14≤6. When e11 is 0, 0≤e12+e13≤4, and when ellis 1, 0≤e12+e13≤6. e4+e12≥1.

In formula (sulfo-1), RFI to R^{F}3 are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Of these, a trifluoromethyl group, a trifluoromethoxy group, and a trifluorothiomethoxy group are preferable. When e5 is 2, 3, or 4, respective RFI may be the same as or different from each other, when e6 is 2, 3, 4, 5, or 6, respective R^{F}2 may be the same as or different from each other, and when e7 is 2, 3, 4, 5, or 6, respective R^{F}3 may be the same as or different from each other.

In formula (sulfo-1), Rall to R^{q14} are a halogen atom other than an iodine atom and a fluorine atom and an iodine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl moiety of the hydrocarbyl group, the hydrocarbyloxy group, and the hydrocarbylthio group may be saturated or unsaturated and straight, branched, or cyclic. Specific examples thereof are as exemplified above for the hydrocarbyl group represented by R¹ in the description of formula (A). Some or all of hydrogen atoms of the hydrocarbyl moiety of the hydrocarbyl group, the hydrocarbyloxy group, and the hydrocarbylthio group may be substituted with a group containing a heteroatom such as an oxygen atom, a sulfur atom, a nitrogen atom, or a halogen atom, and some of —CH₂— of the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), or a haloalkyl group.

When c8 is 2, two R^{q11}'s may be the same as or different from each other, two R^{q11}'s may bond together to form a ring with the carbon atom to which they are attached, when e9 is 2, two R^{q12}'s may be the same as or different from each other, two R^{q12}'s may bond together to form a ring with the carbon atom to which they are attached, when e10 is 2, two R^{q13}'s may be the same as or different from each other, two R^{q13}'s may bond together to form a ring with the carbon atom to which they are attached, and when e13 is 2, two R^{q14}'s may be the same as or different from each other, two R^{q14}'s may bond together to form a ring with the carbon atom to which they are attached. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring may be substituted with a group containing a heteroatom such as an oxygen atom, a sulfur atom, a nitrogen atom, or a halogen atom, some of—CH₂-in the ring may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a carbonyl group, an ether bond, an ester bond, a sulfonic acid ester bond, a carbonate bond. a lactone ring. a sultone ring, a carboxylic anhydride (—C(═O)—O—C(═O)—). a haloalkyl group, or the like.

Aromatic rings directly bonded to S⁺in the sulfonium cation having the formula (sulfo-1) may bond together to form a ring with S⁺. In this case, specific examples of the structure of the ring are those having the following formula:

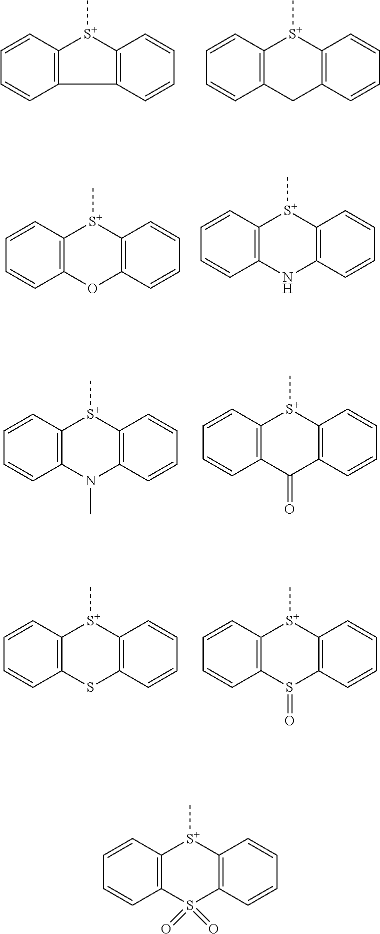

wherein the broken line denotes a point of attachment.

In formula (sulfo-1), L^{F} and L^{G} are each independently a single bond, an ether bond, an ester bond, an amide bond, a sulfonic acid ester bond, a sulfonic acid amide bond, a carbonate bond, or a carbamate bond. Of these, L^{F} is preferably a single bond, an ether bond, an ester bond, or a sulfonic acid ester bond, and more preferably an ester bond or a sulfonic acid ester bond. L^{G} is preferably a single bond, an ether bond, or an ester bond, and more preferably a single bond.

In formula (sulfo-1), X^{L3} is a single bond or a C₁-C₄₀ hydrocarbylene group which may contain a heteroatom. The hydrocarbylene group may be straight, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the C₁-C₄₀ hydrocarbylene group, which may contain a heteroatom, represented by X¹³ include X^{L}-0 to X^{L}-58 exemplified as specific examples of the C₁-C₄₀ hydrocarbylene group, which may contain a heteroatom, represented by X^{L1} and X¹² in the description of formula (Z). Of these, X^{L3} is preferably X^{L}-0 to X^{L}-22, X^{L}-29 to X^{L}-34, and X^{L}-47 to X^{L}-58.

The sulfonium cation having formula (sulfo-1) preferably has the following formula (sulfo-1-1):

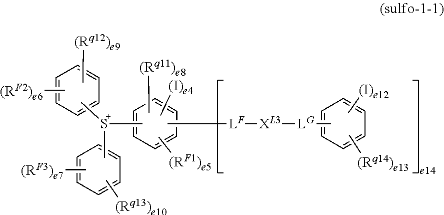

wherein e4 to e10, e12 to e14, R^{F1} to R^{F3}, R^{q11} to R^{q14}, L^{F}, L^{G}, and X^{L3} are as defined above.

The sulfonium cation having formula (sulfo-1-1) preferably has the following formula (sulfo-1-2):

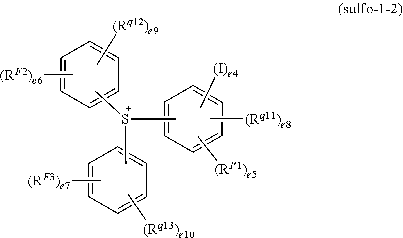

wherein e4 to e10, R^{F1} to R^{F3}, and R^{q11} to R^{q13} are as defined above.

Specific examples of the sulfonium cation having formula (sulfo-1) are shown below. but not limited thereto. In the following formula. Me is a methyl group.

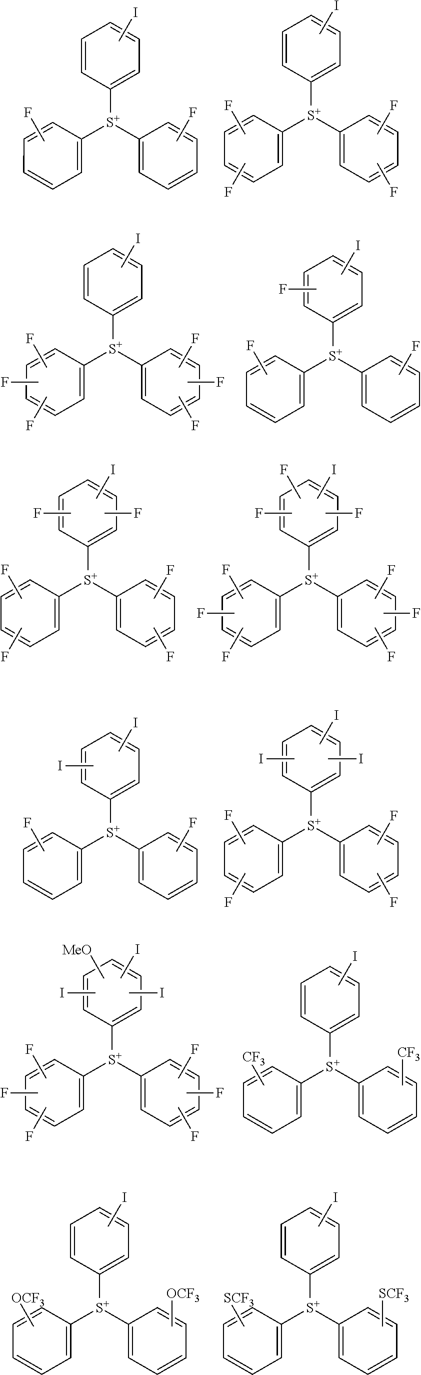

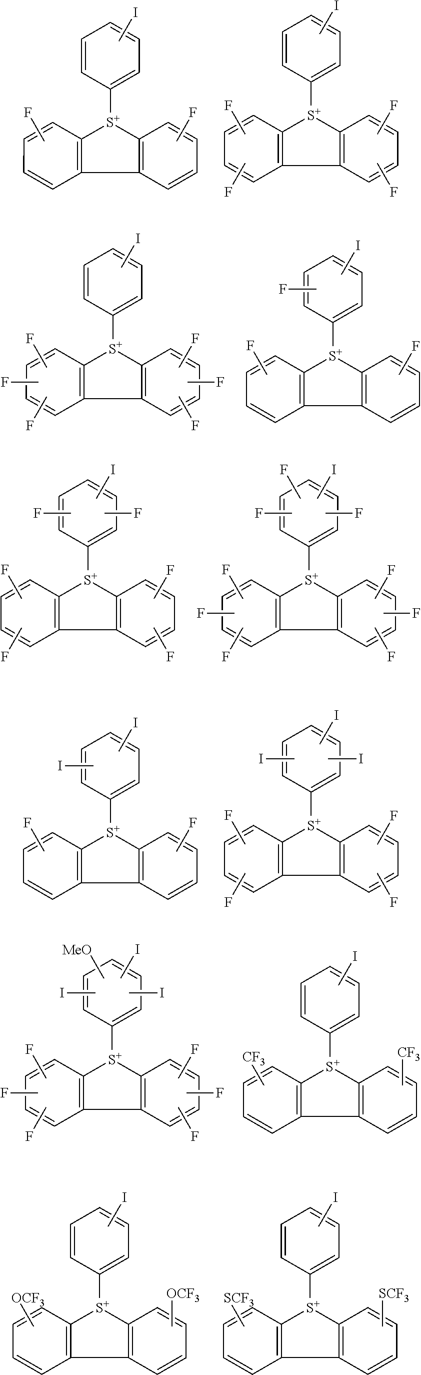

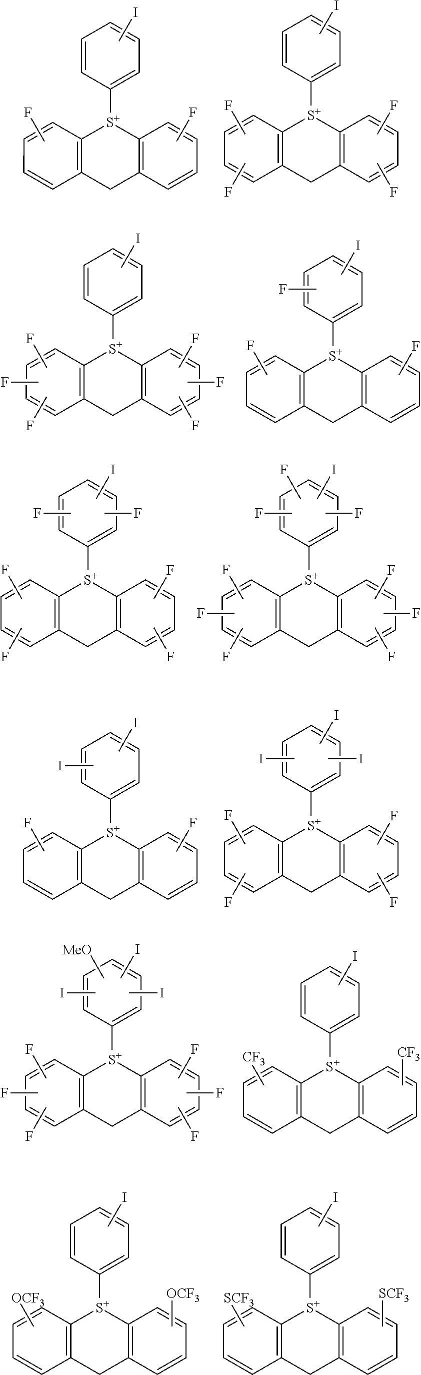

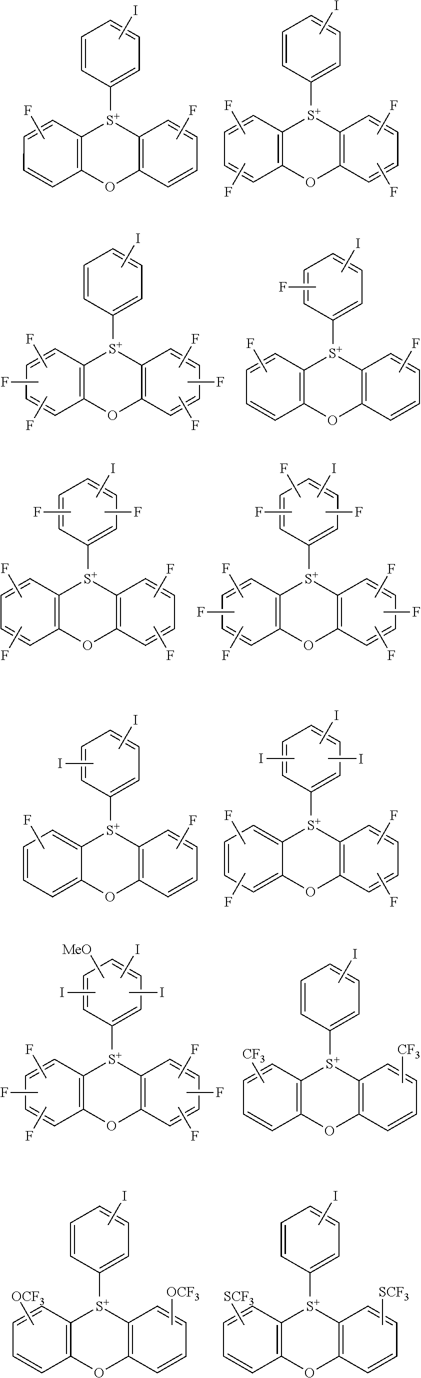

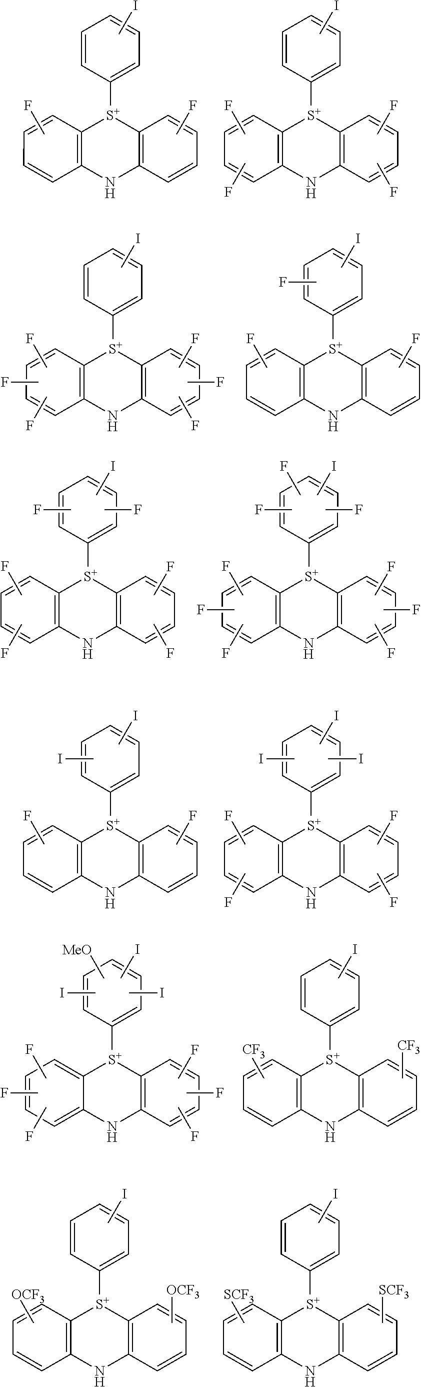

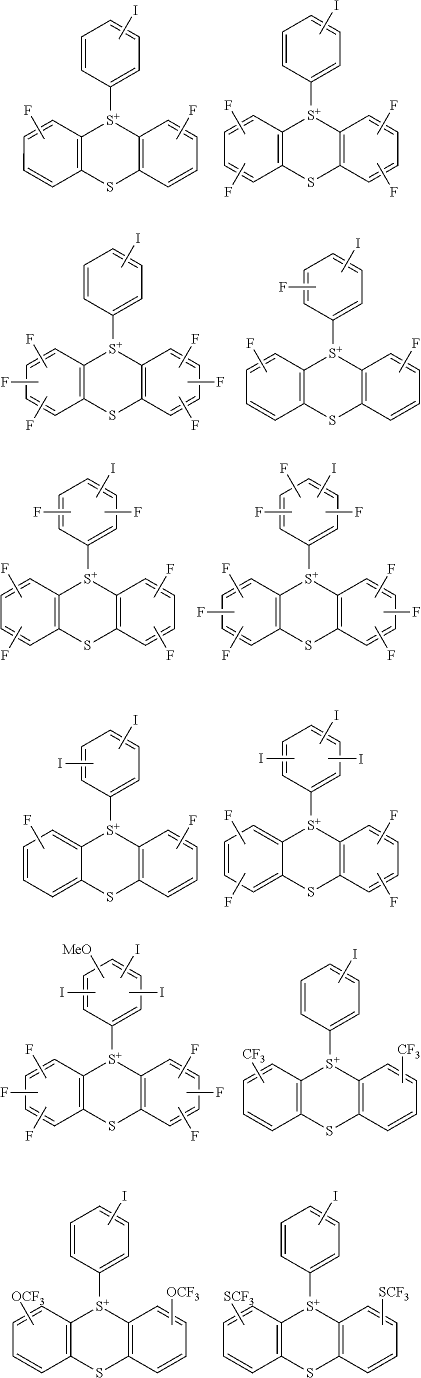

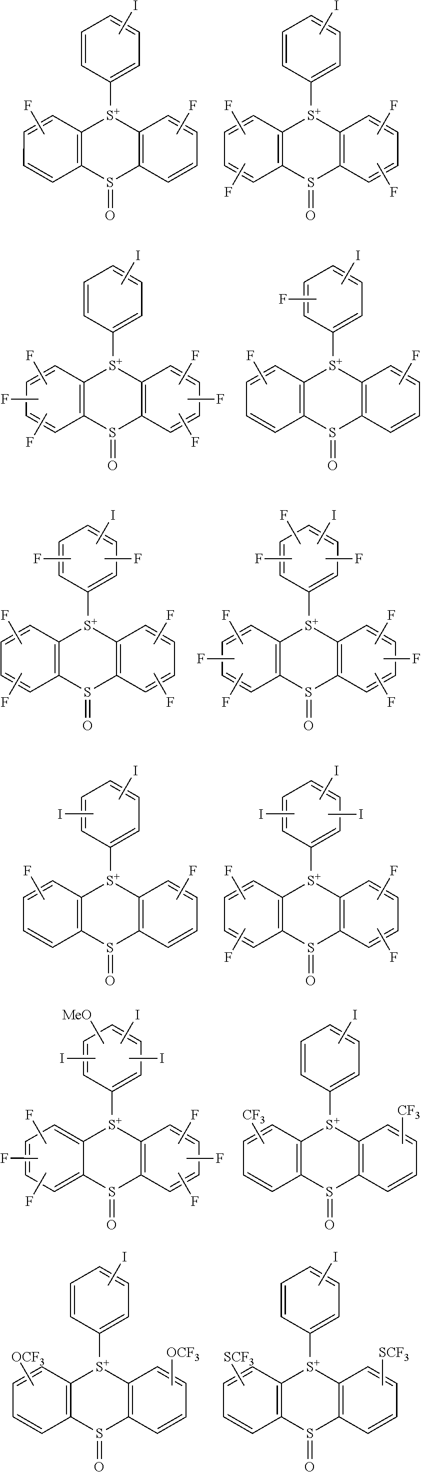

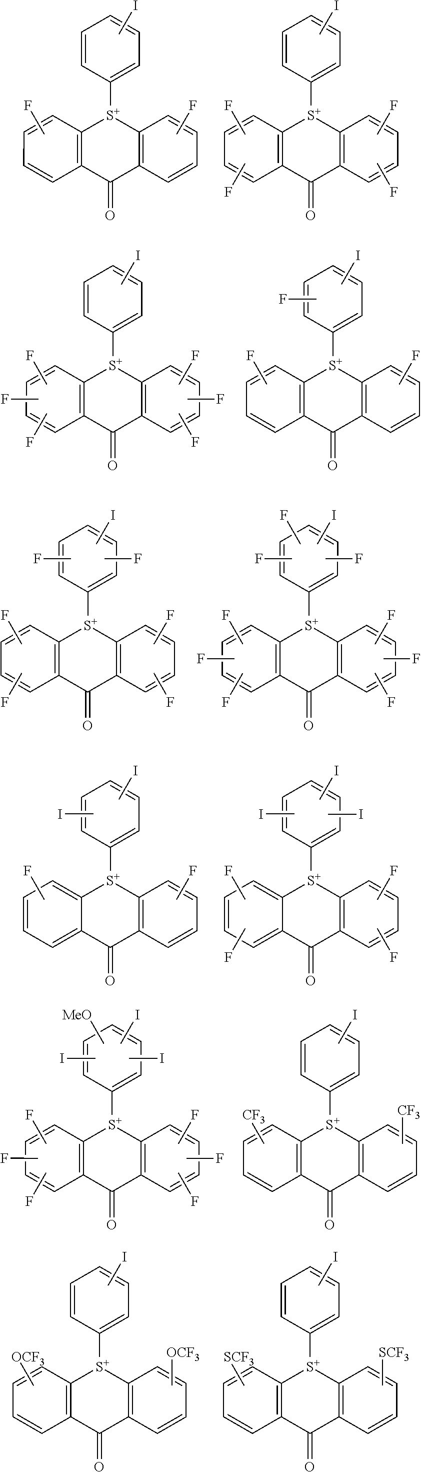

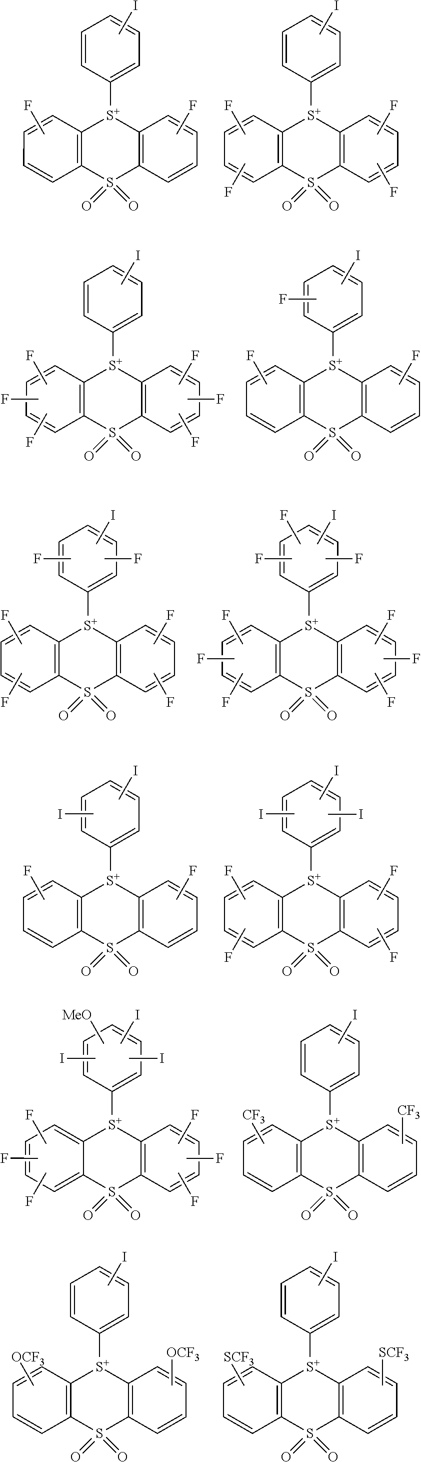

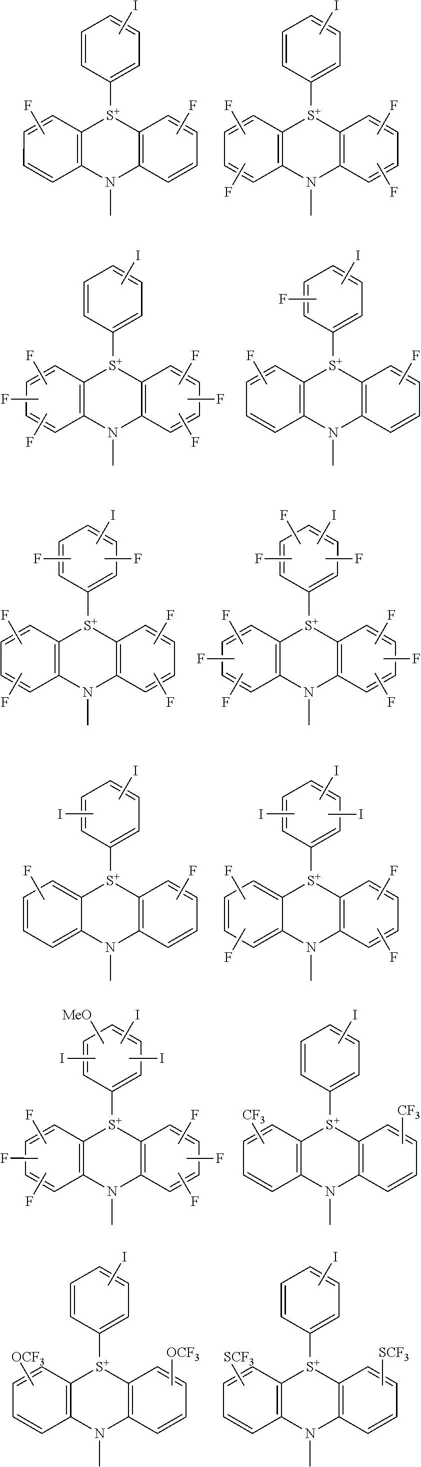

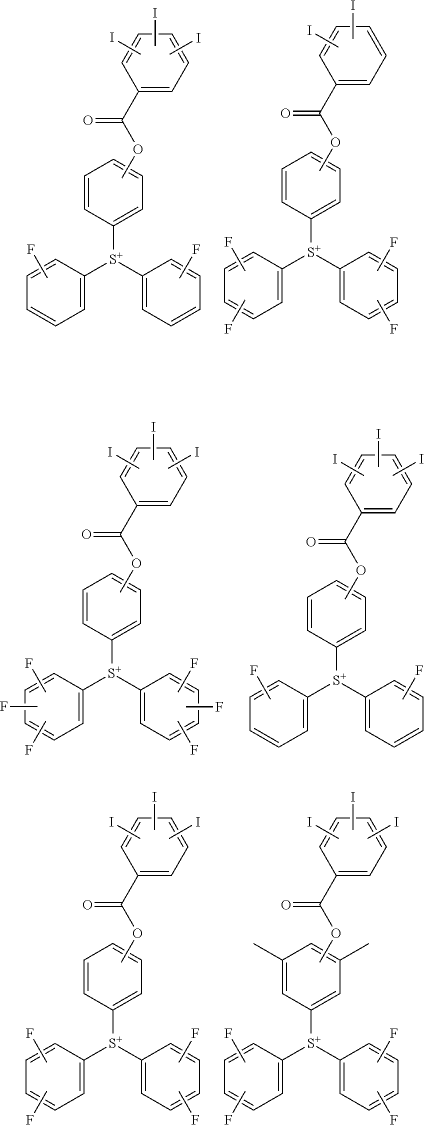

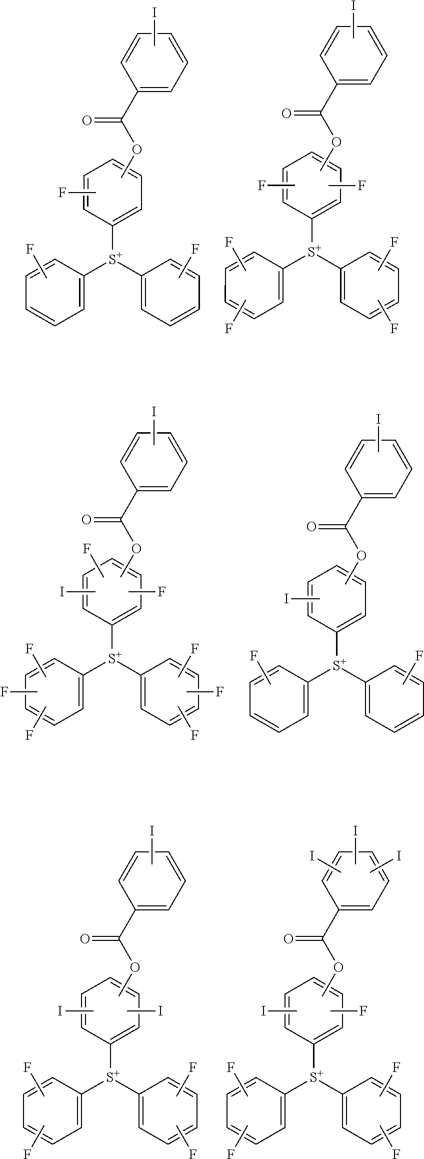

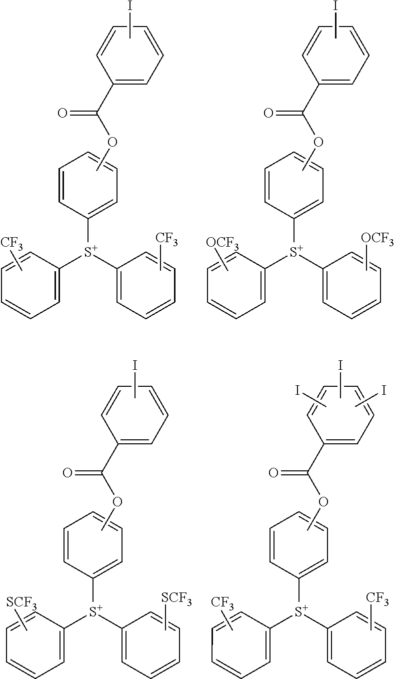

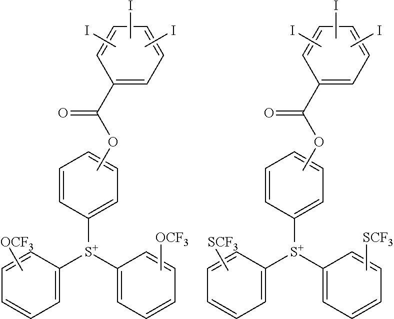

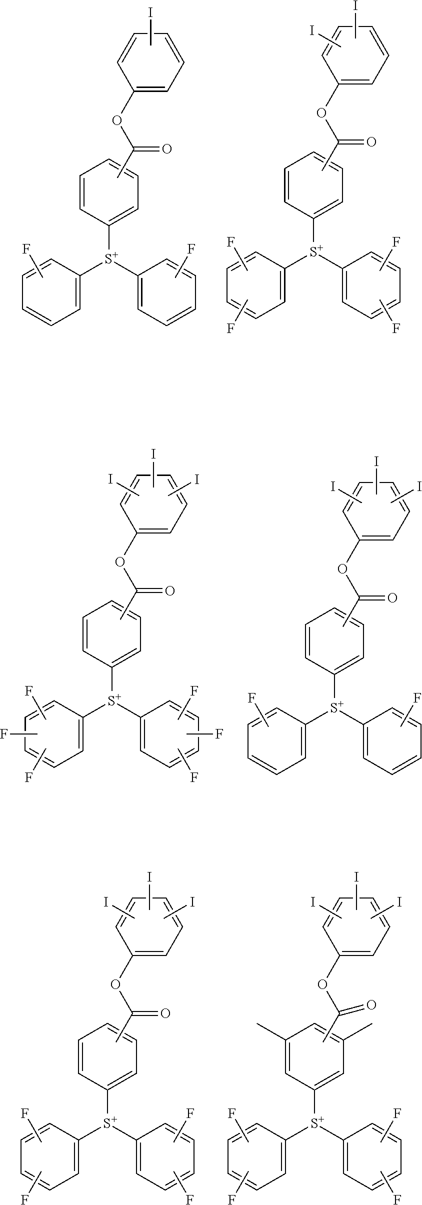

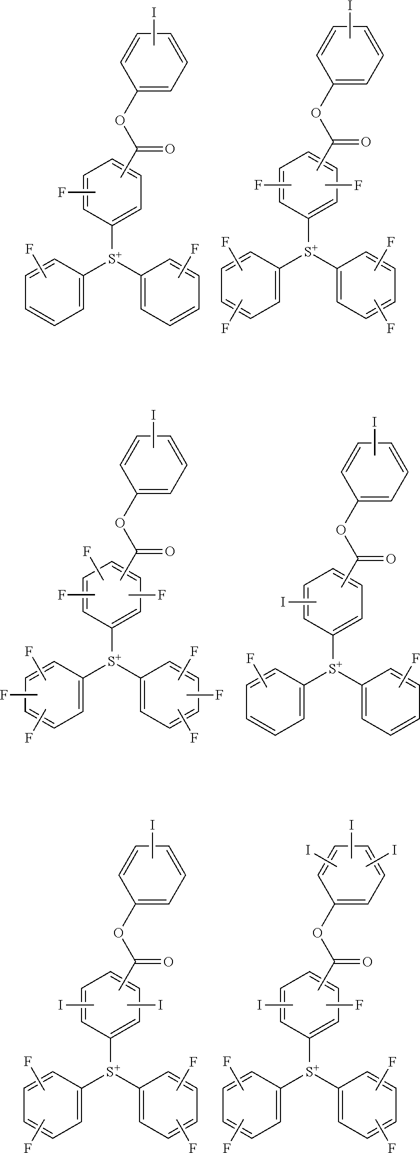

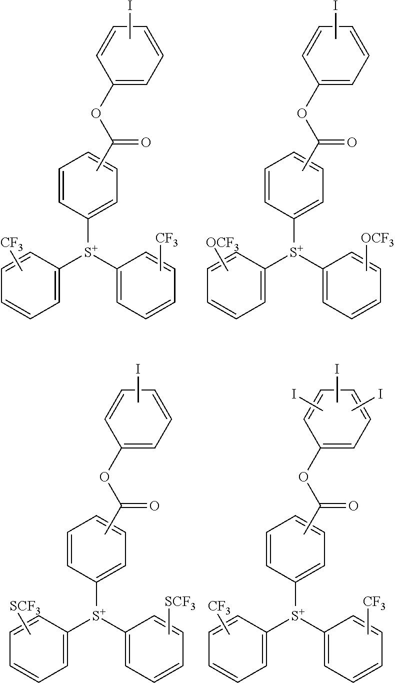

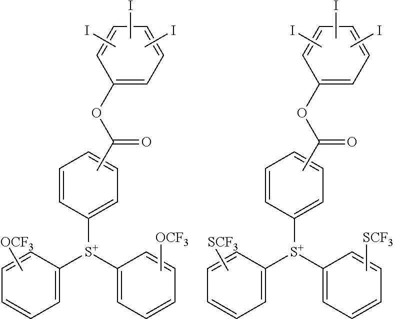

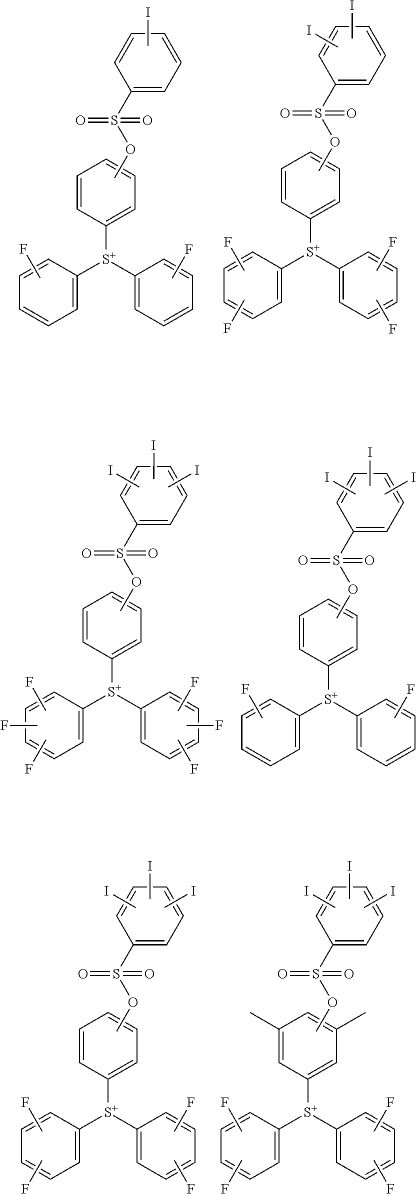

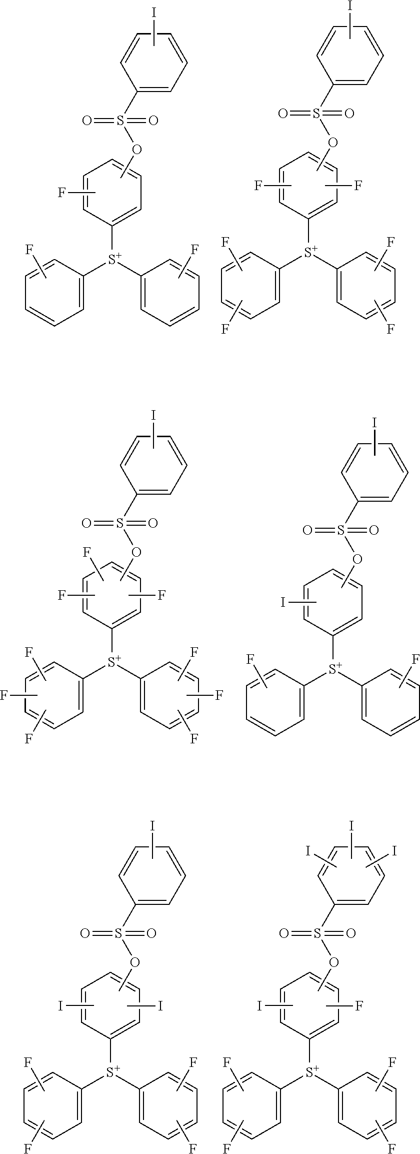

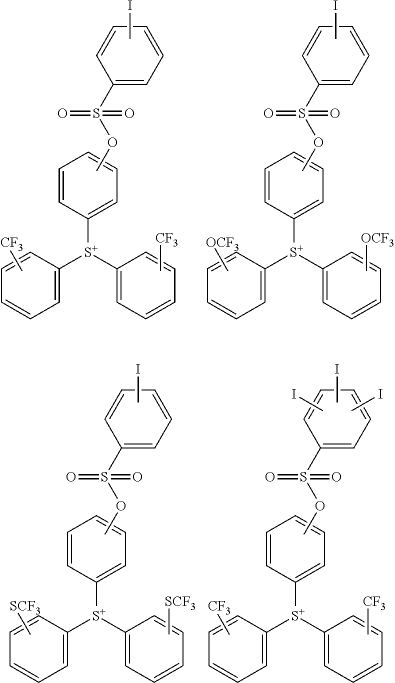

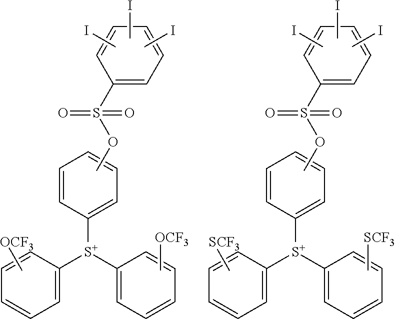

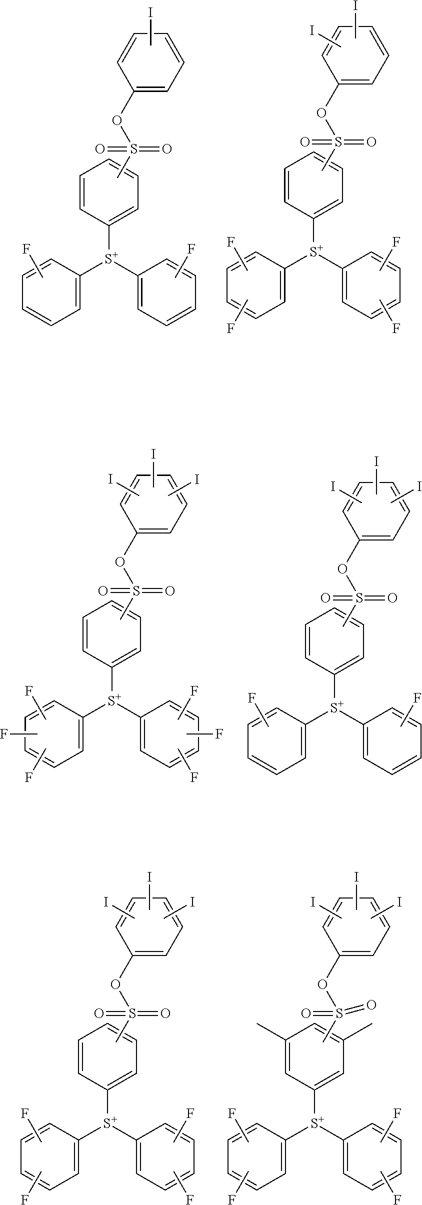

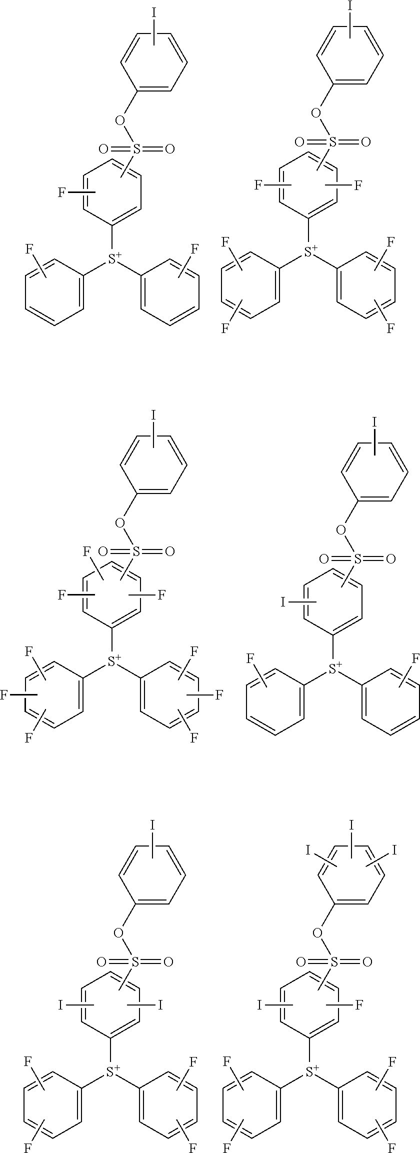

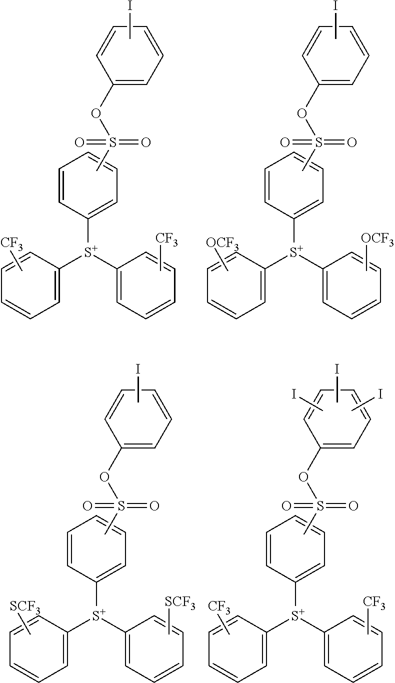

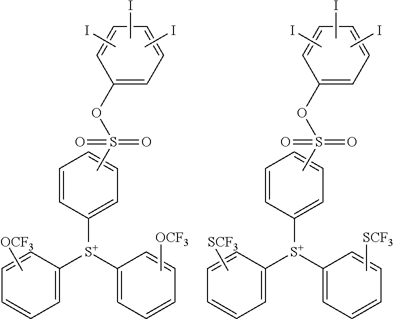

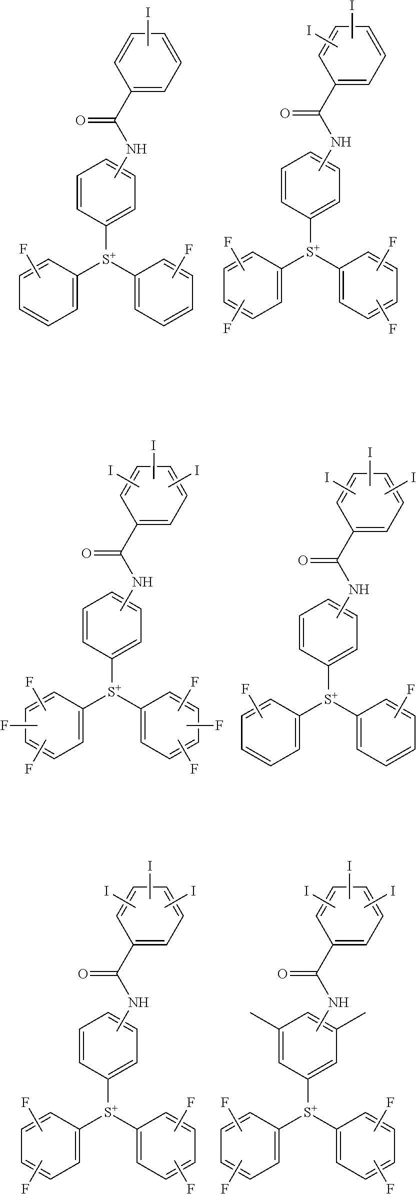

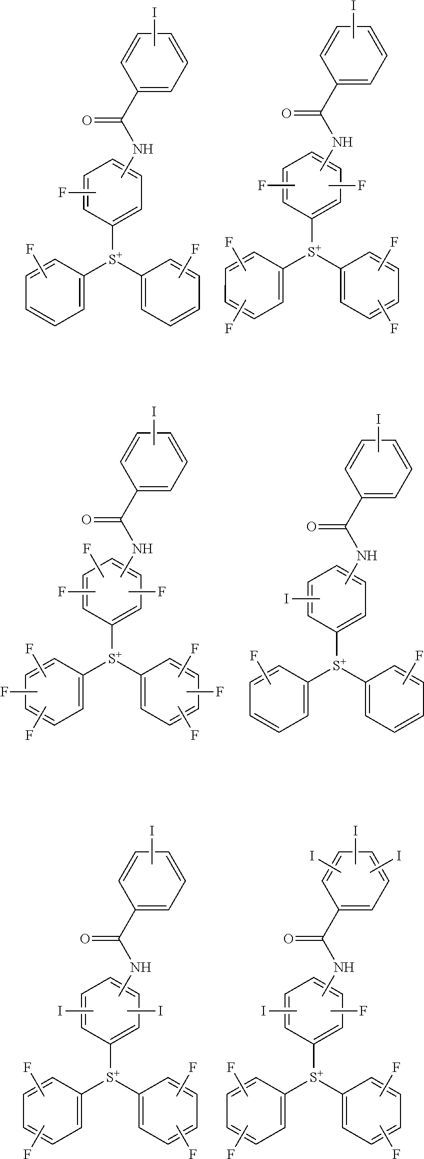

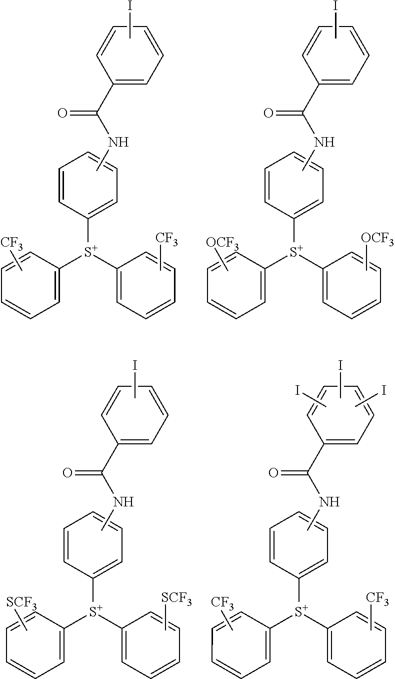

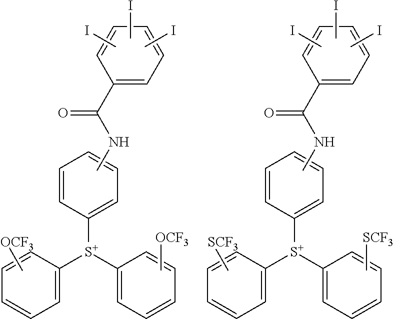

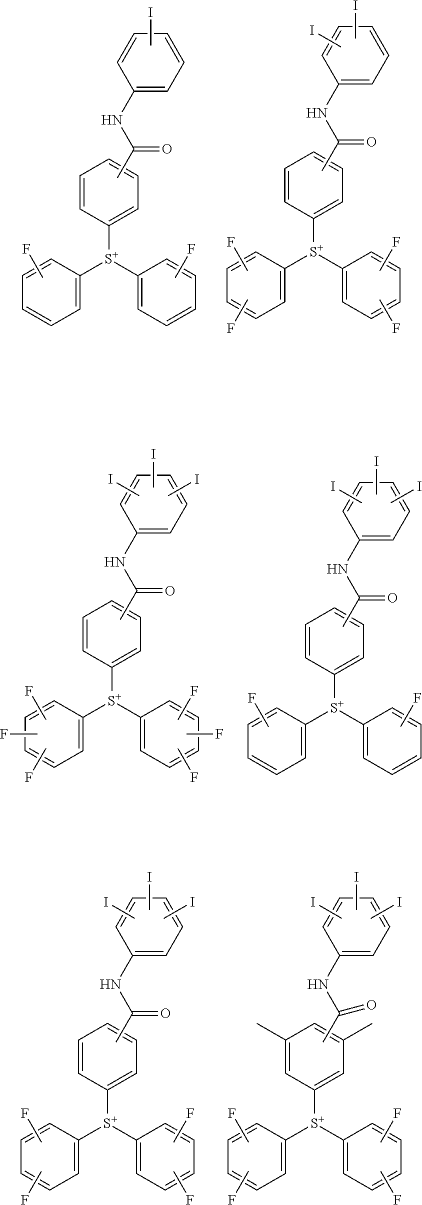

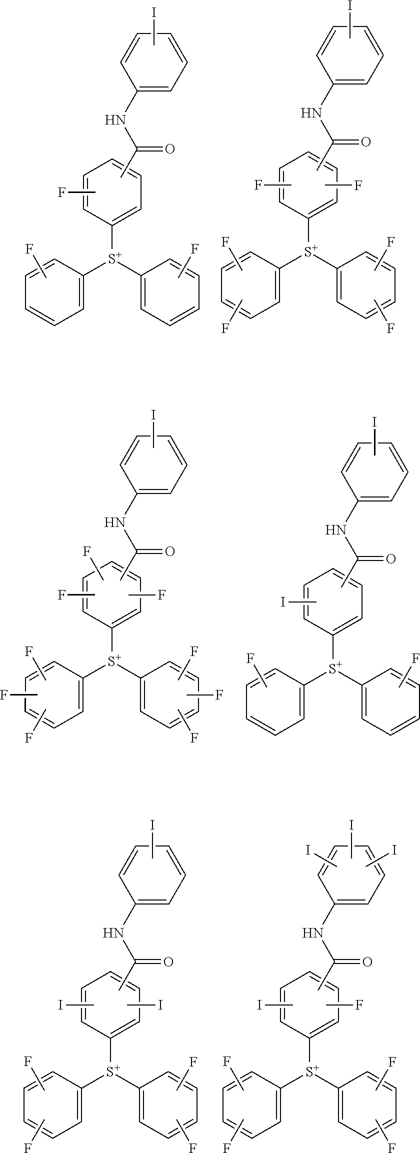

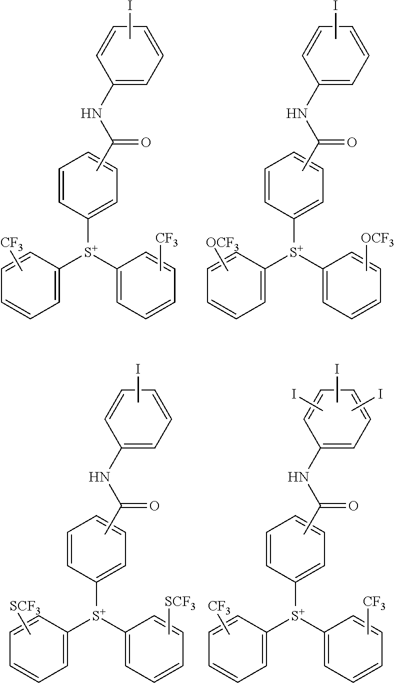

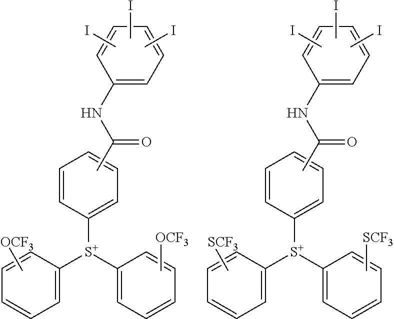

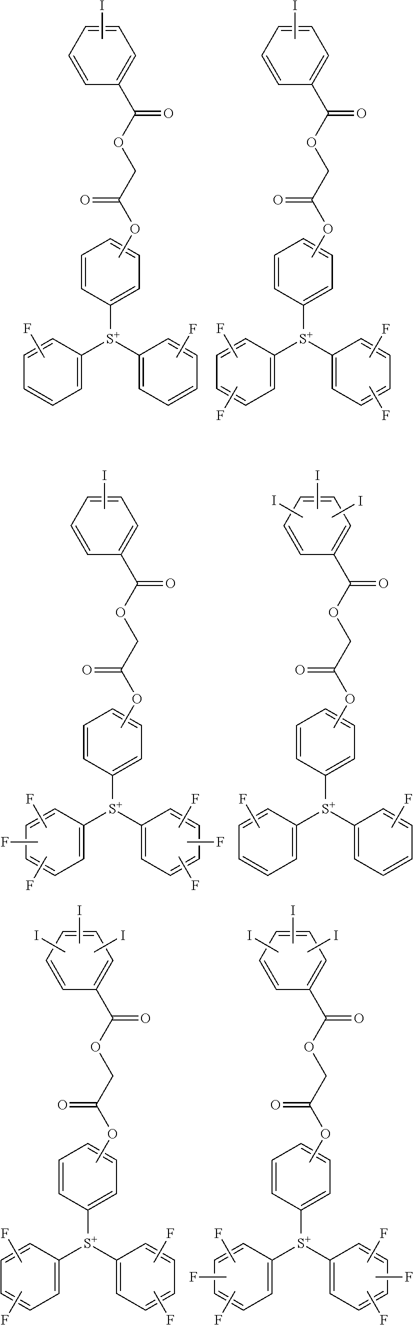

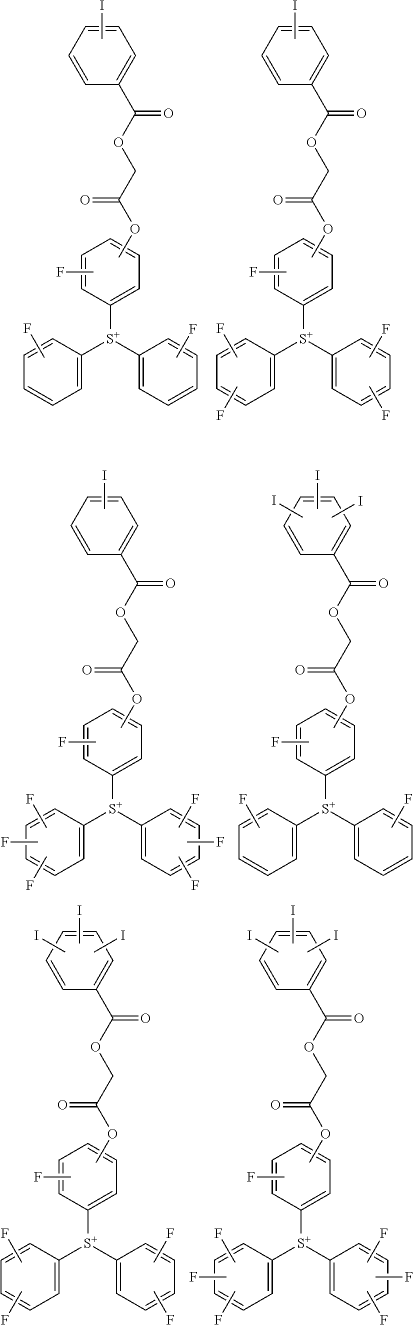

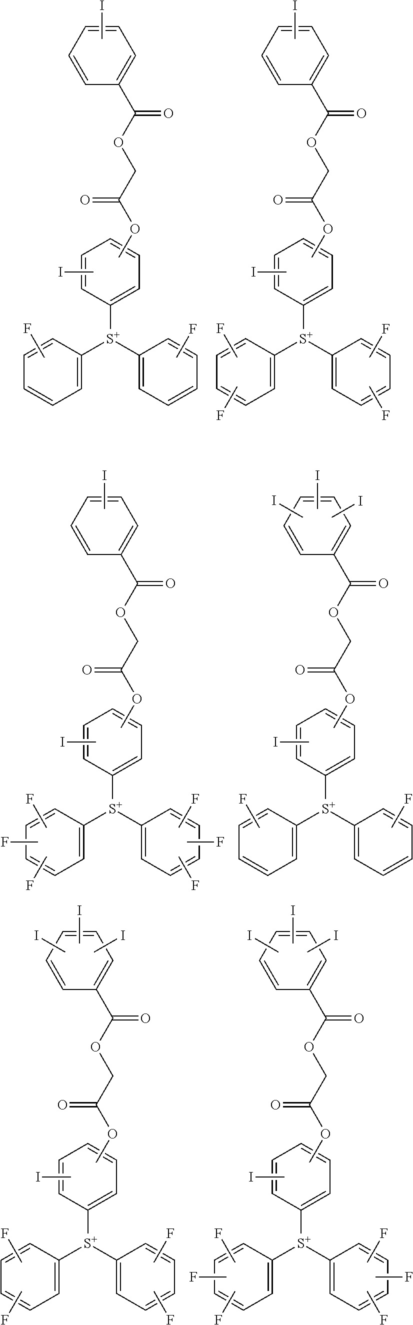

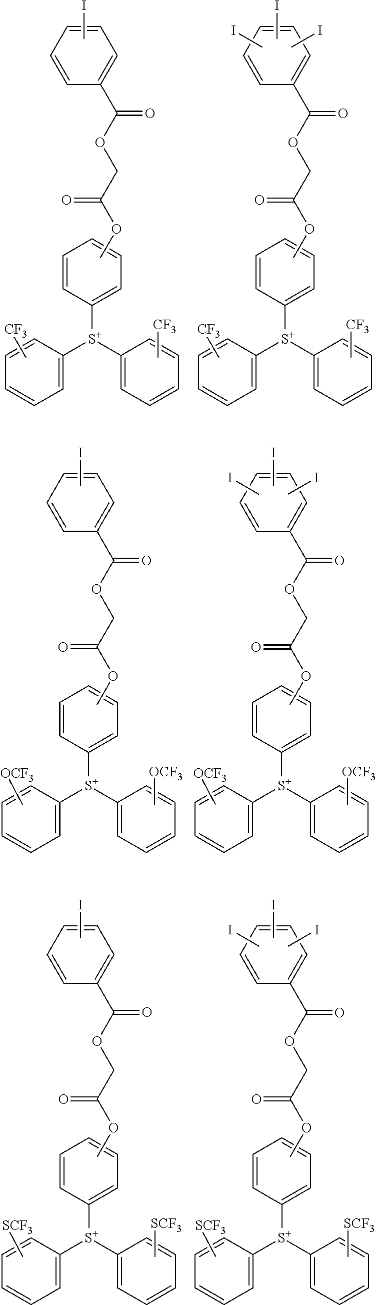

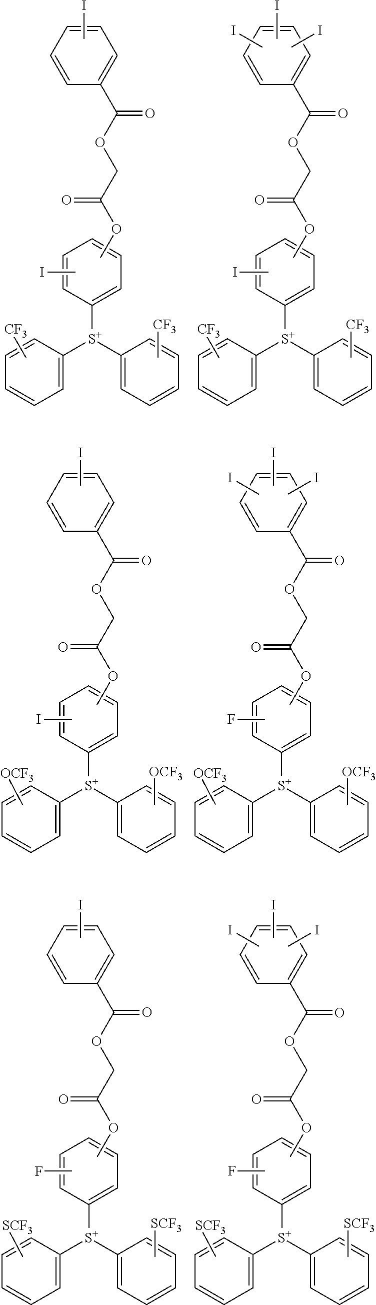

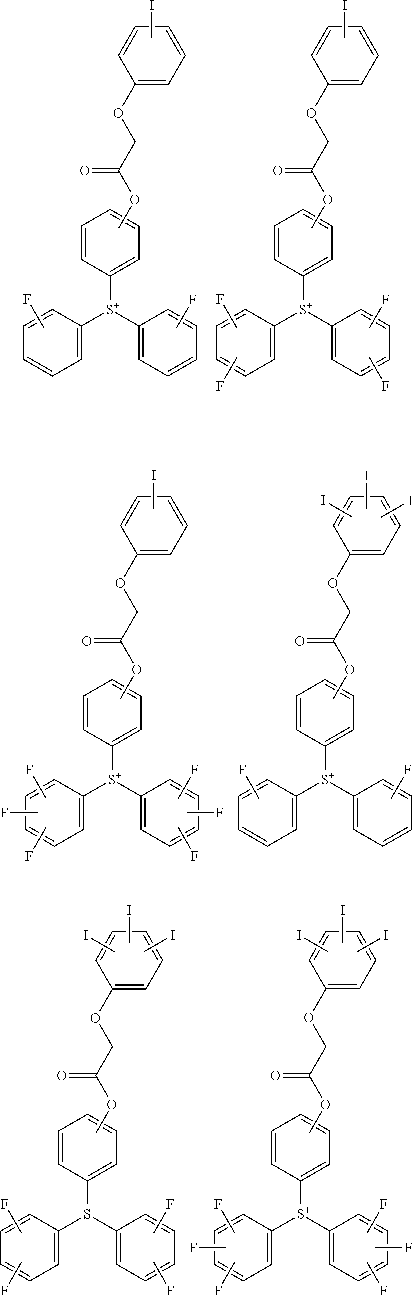

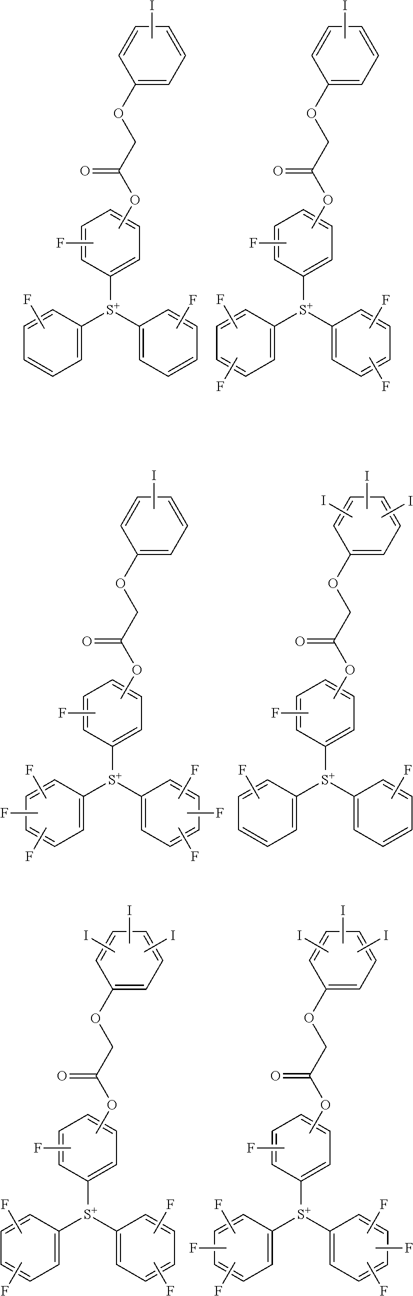

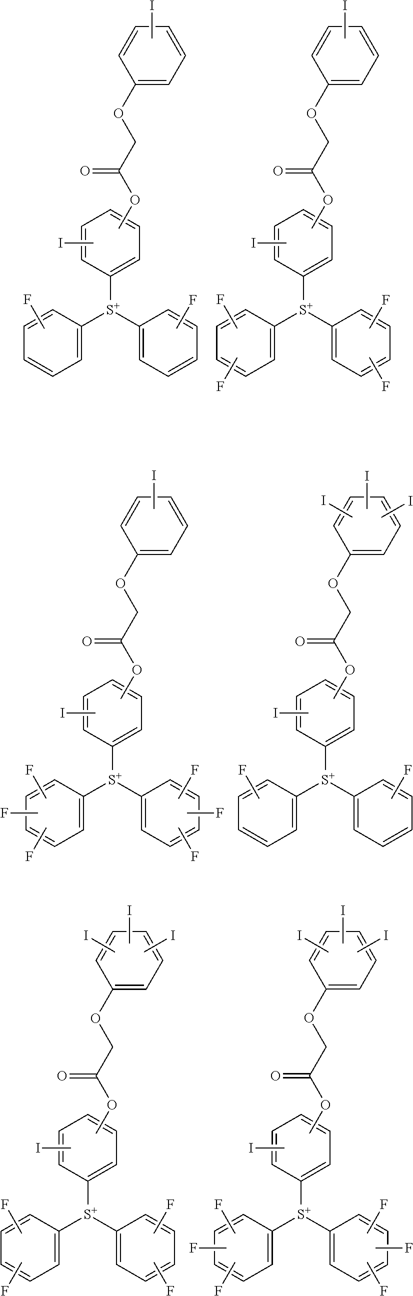

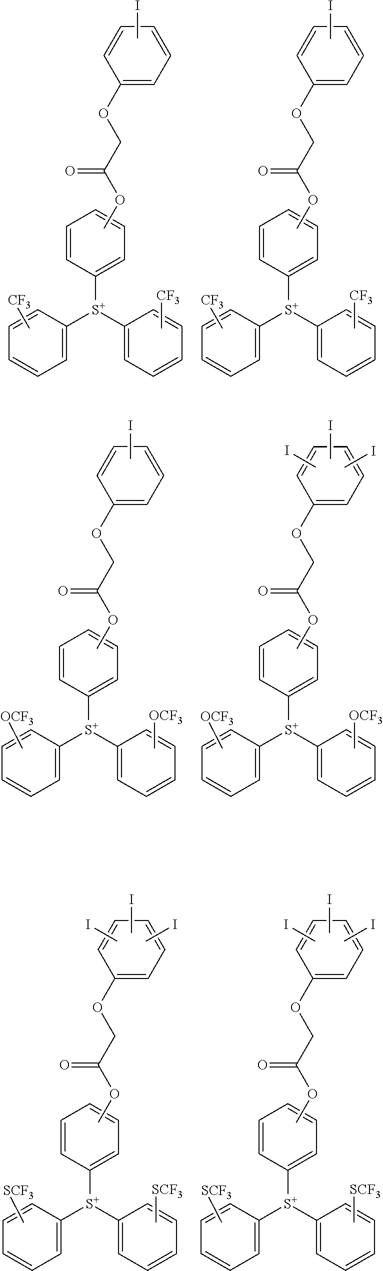

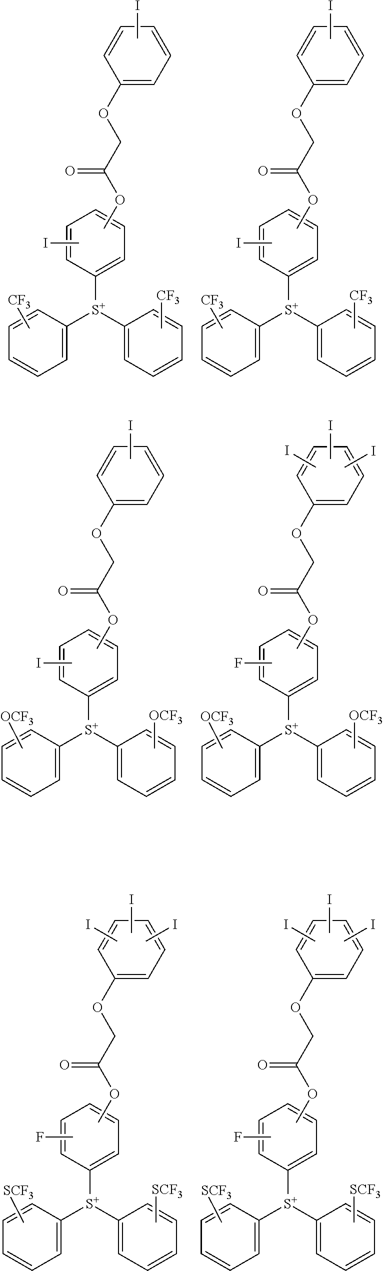

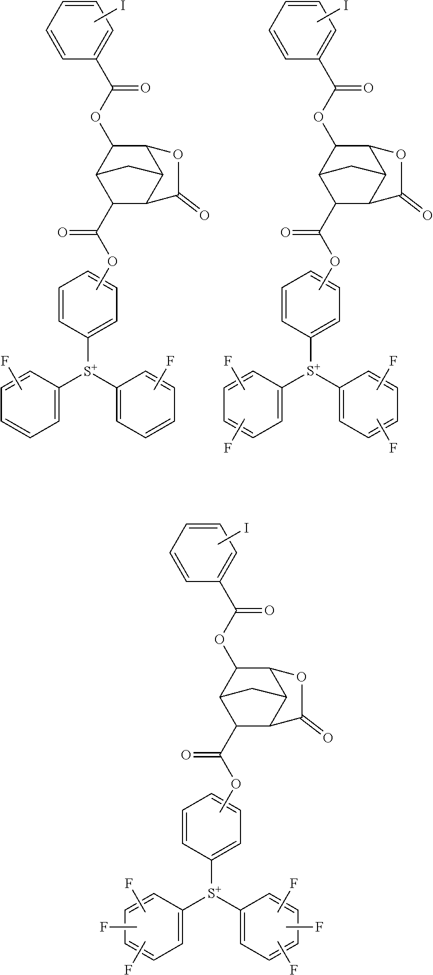

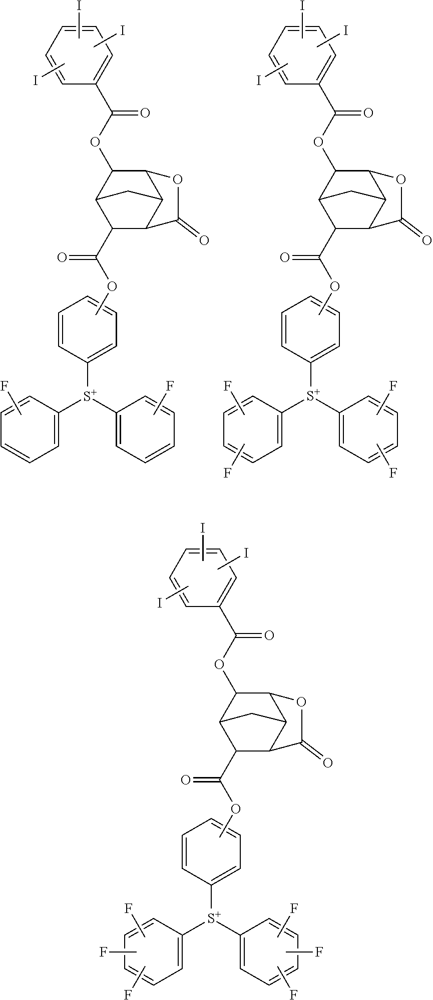

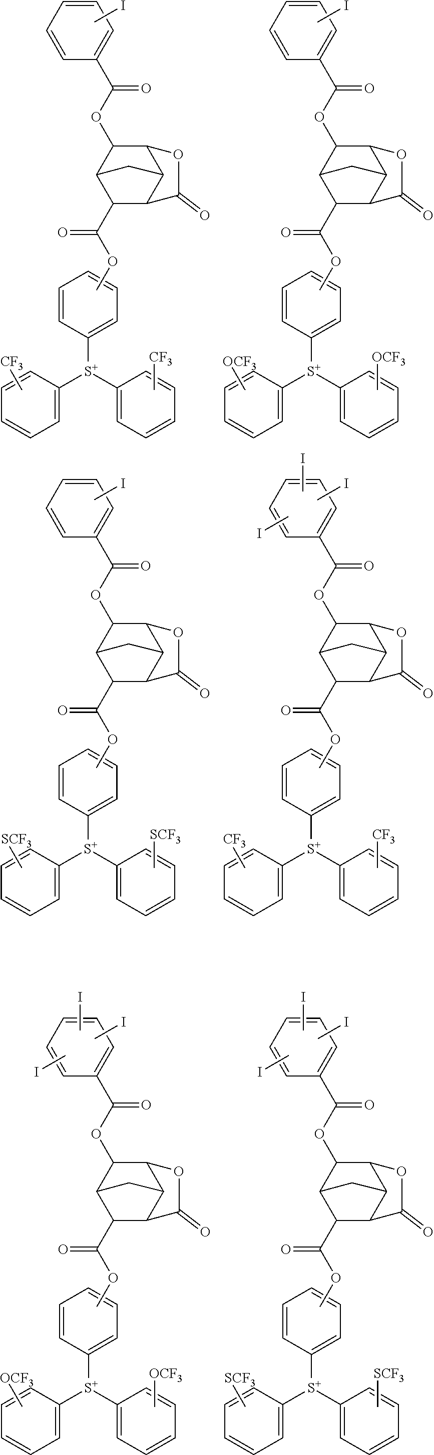

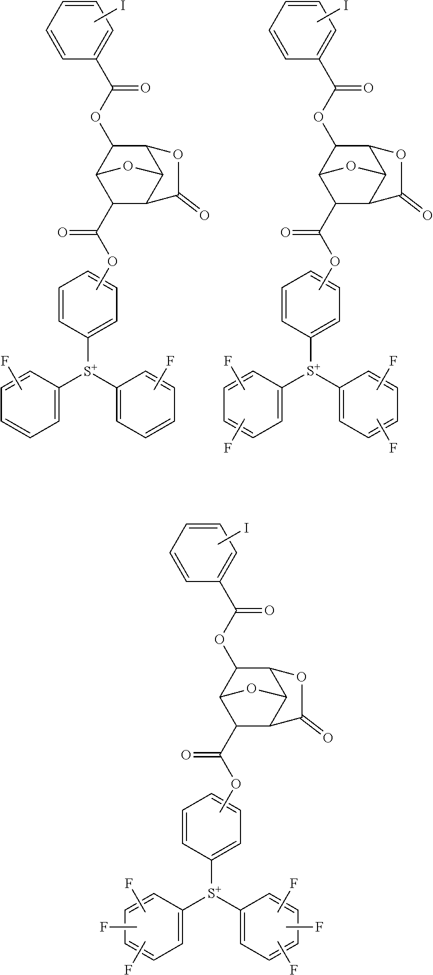

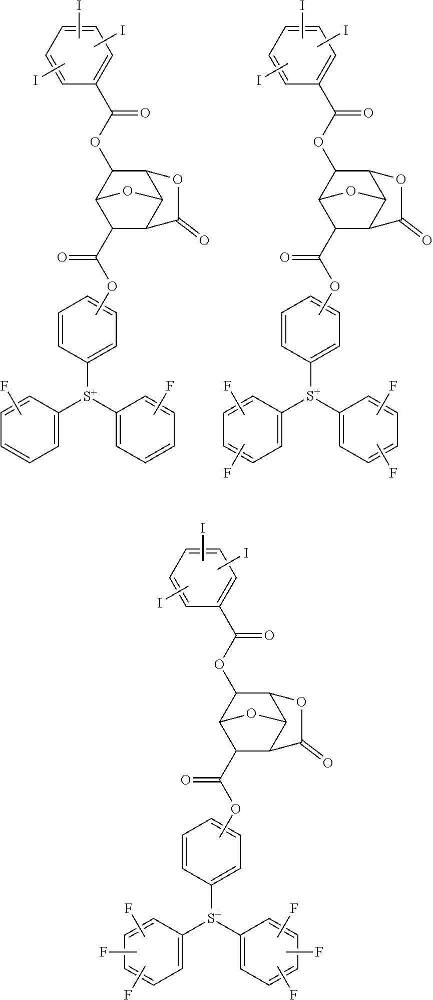

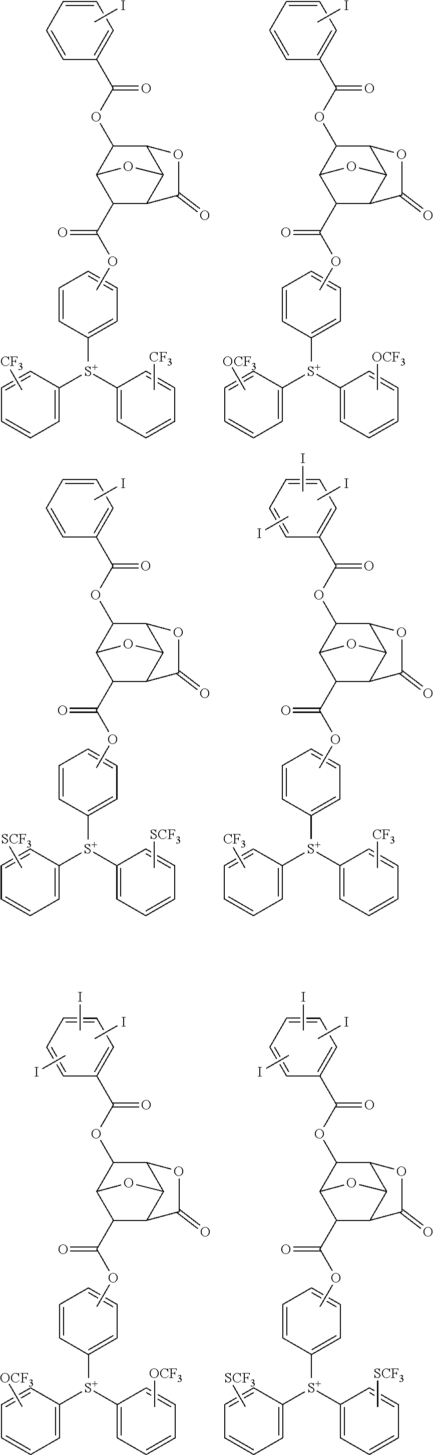

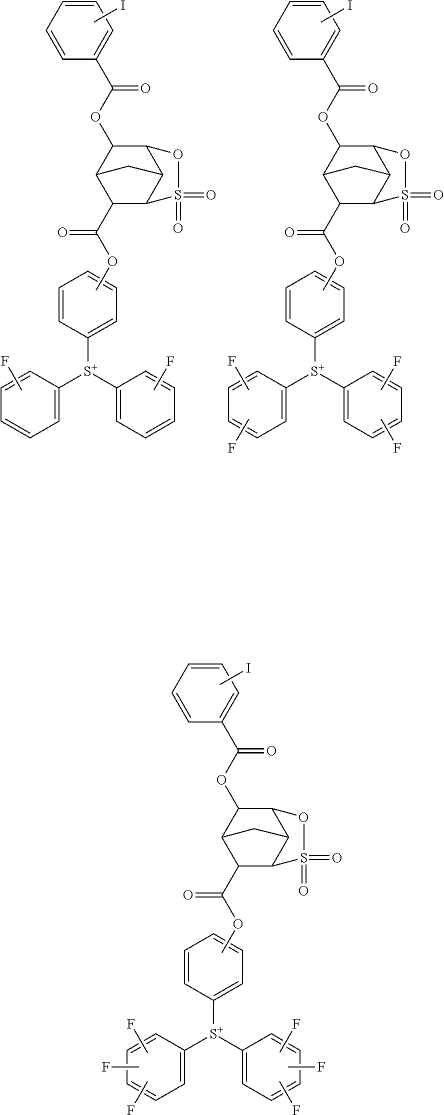

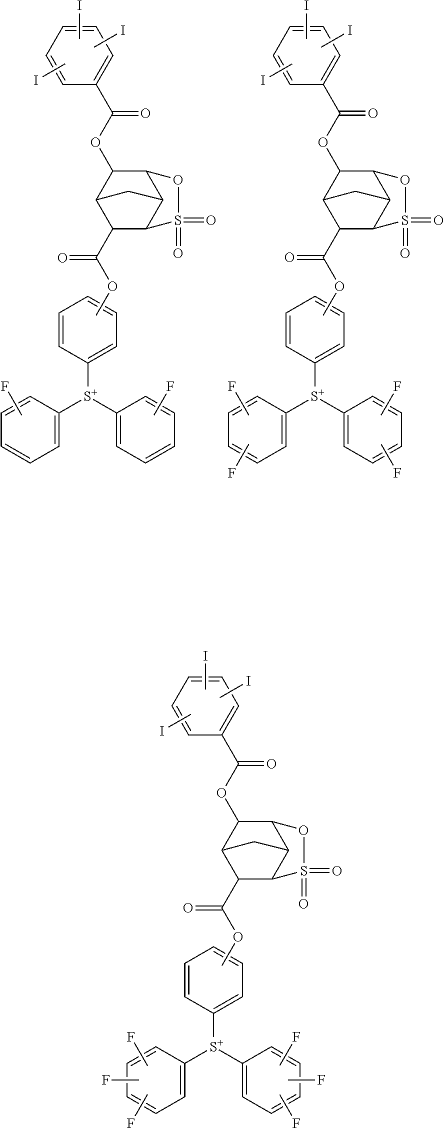

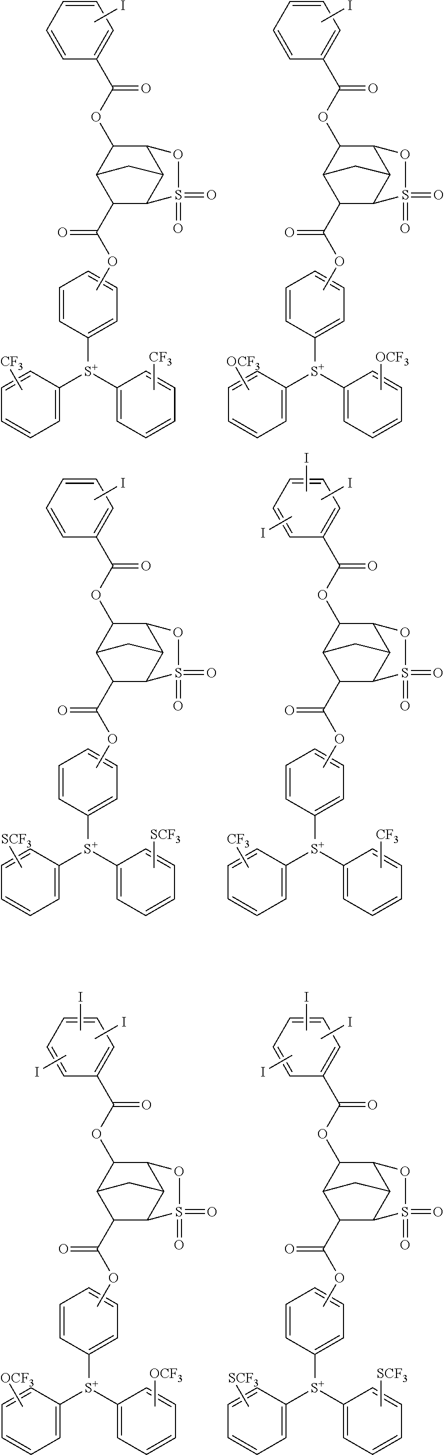

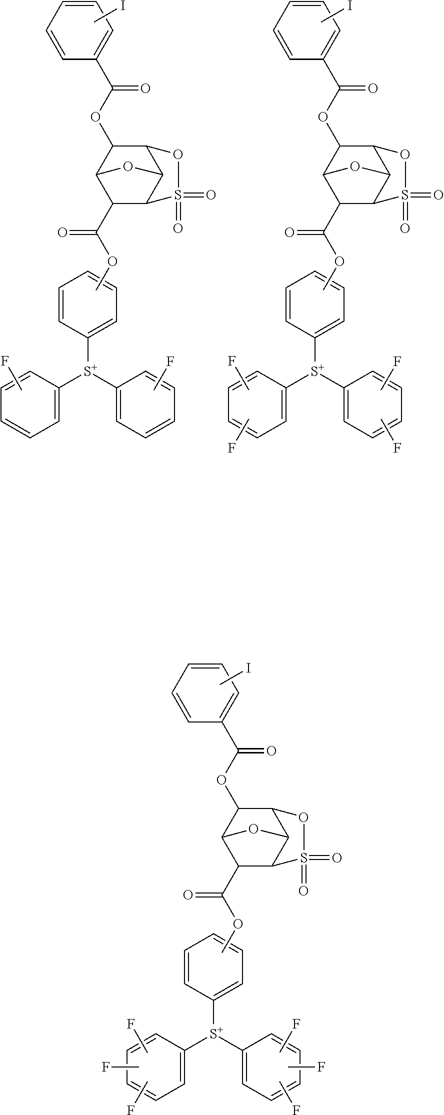

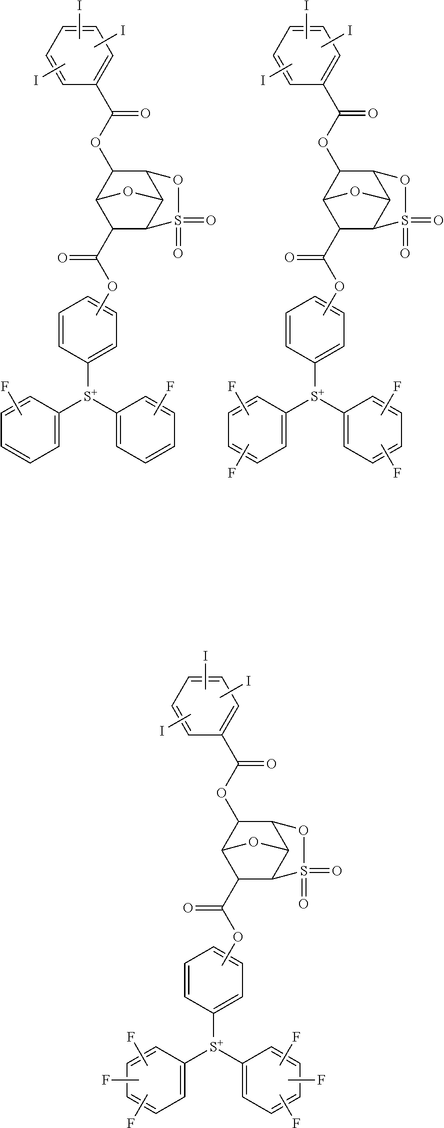

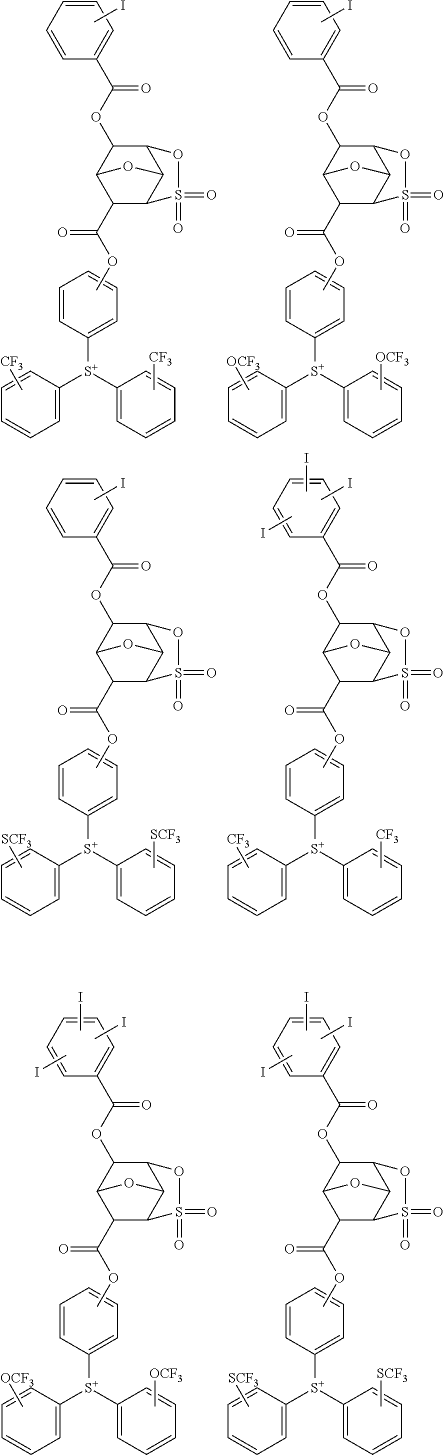

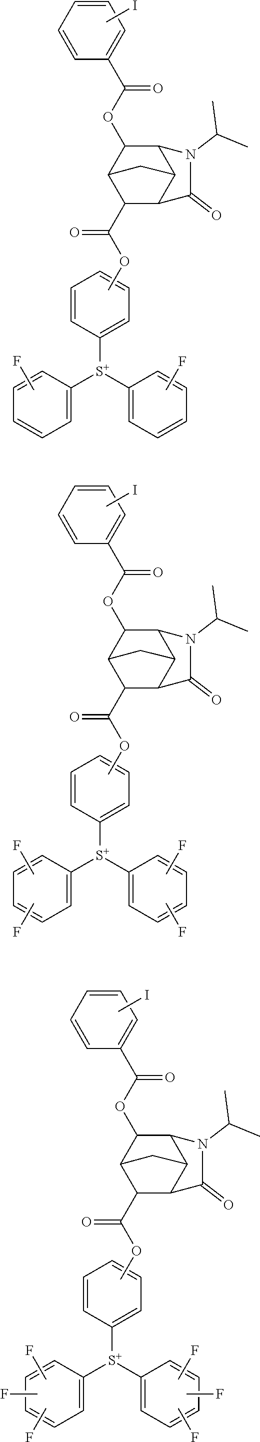

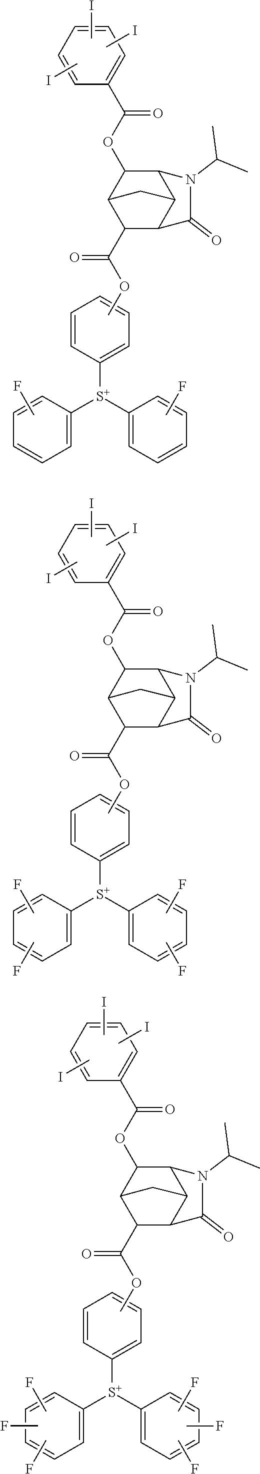

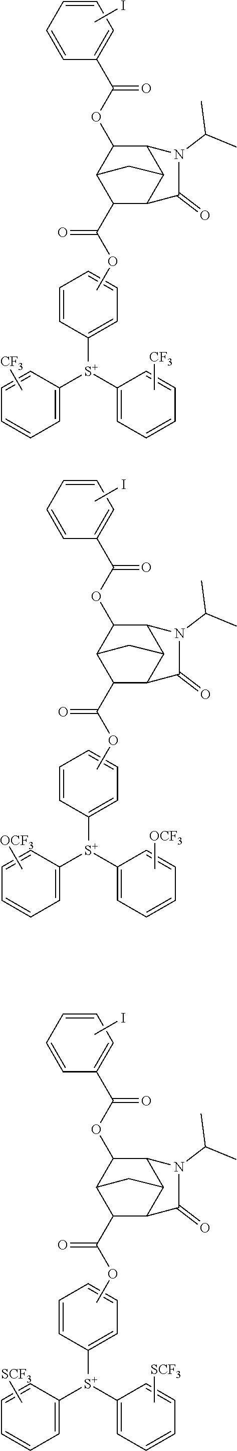

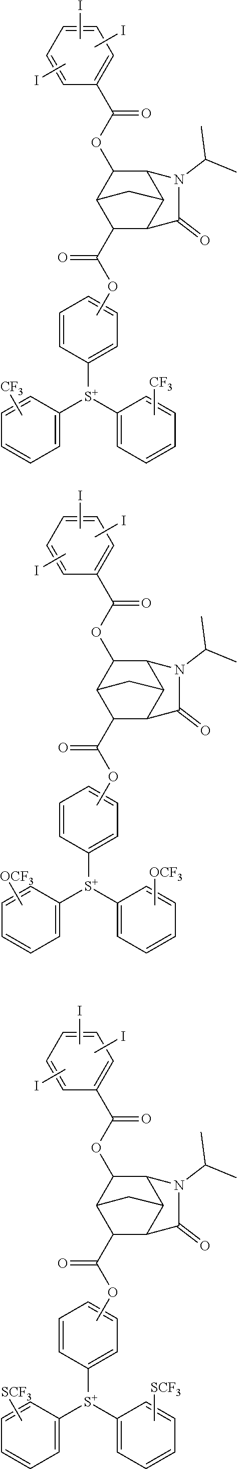

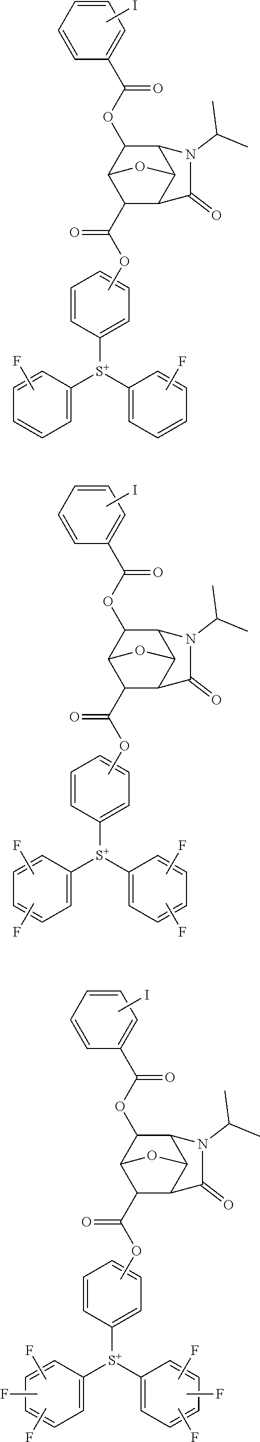

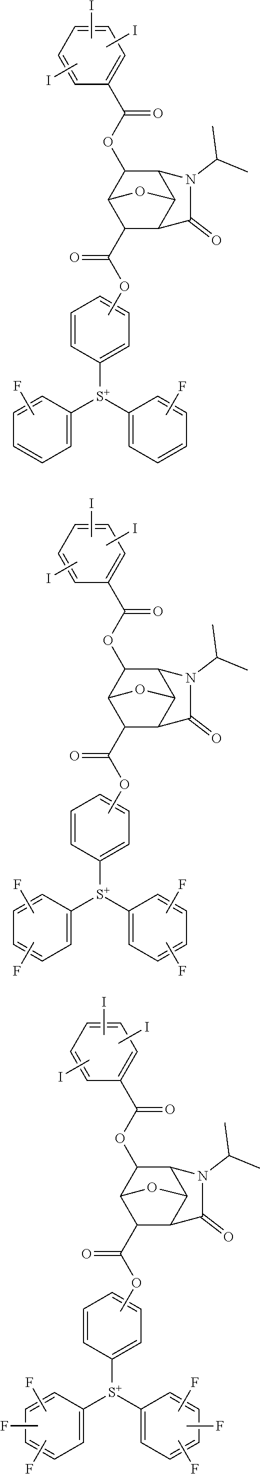

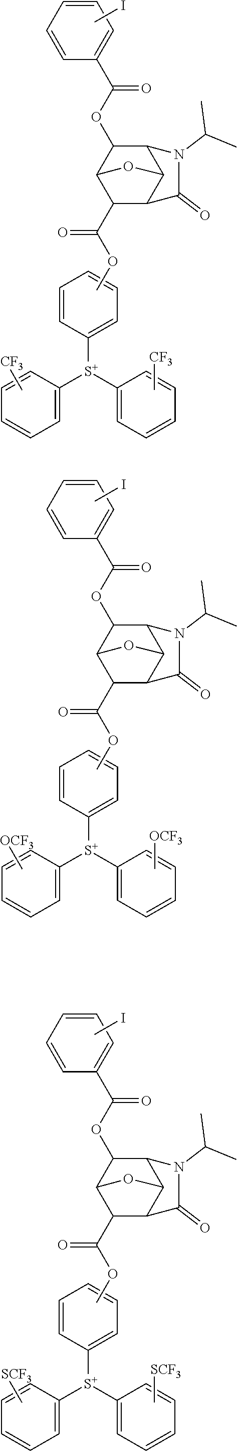

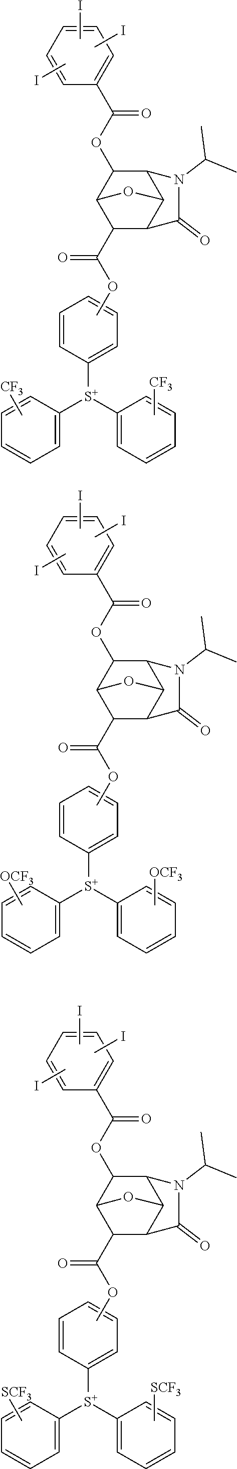

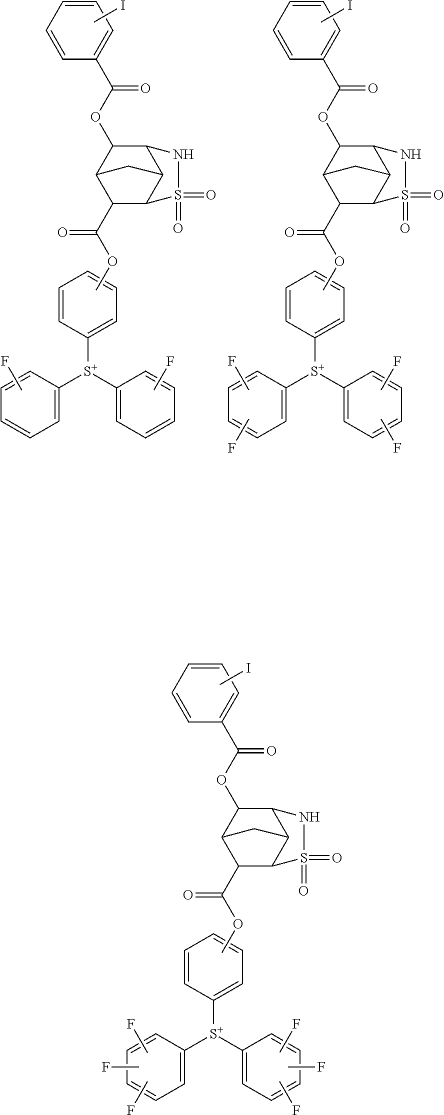

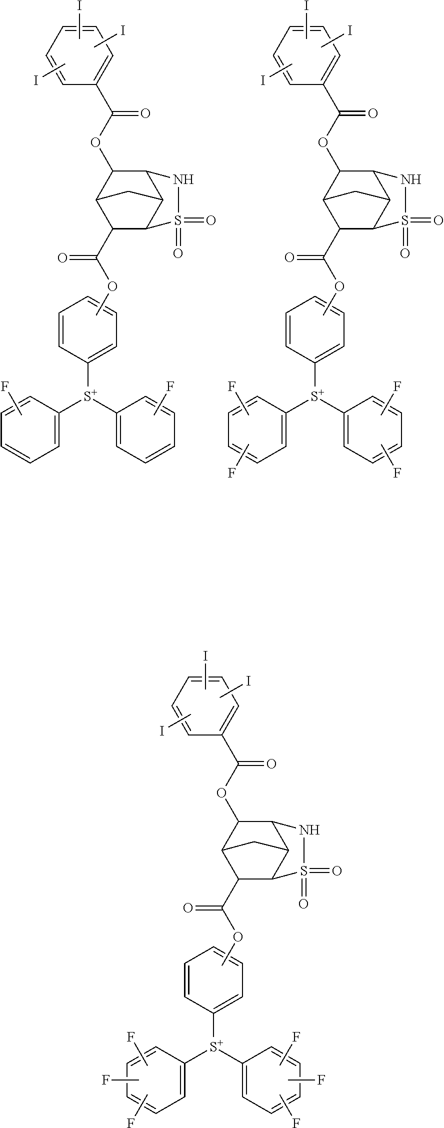

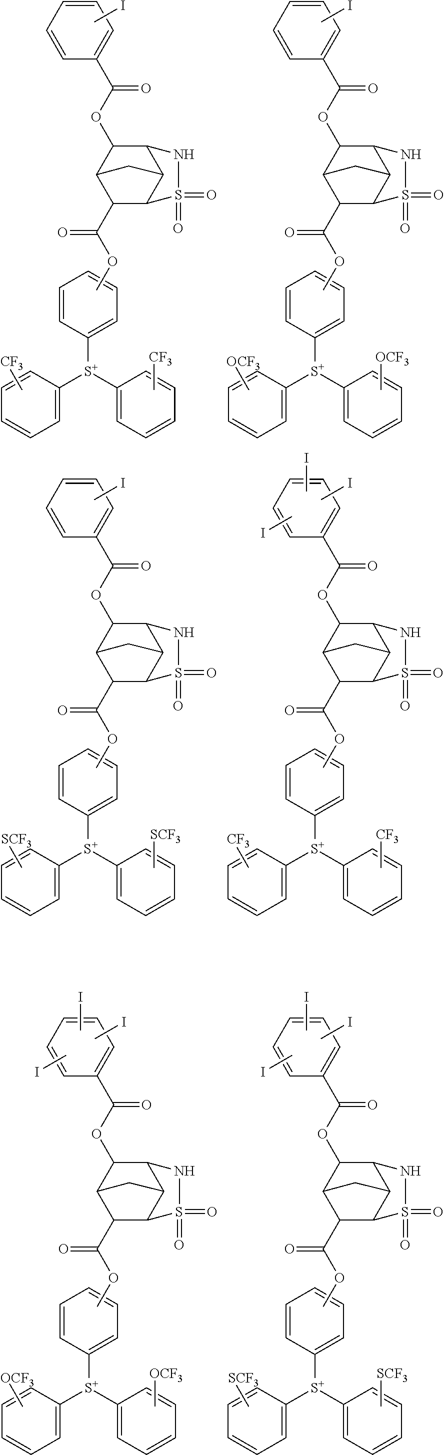

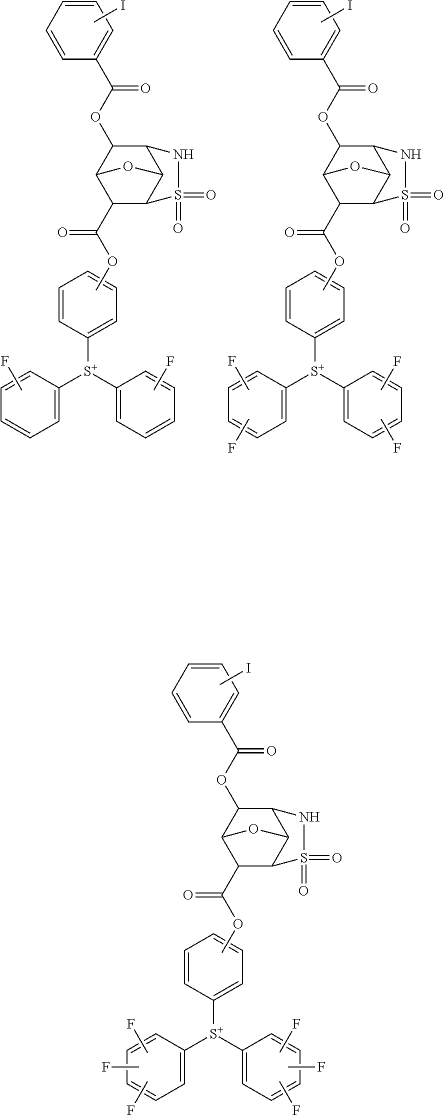

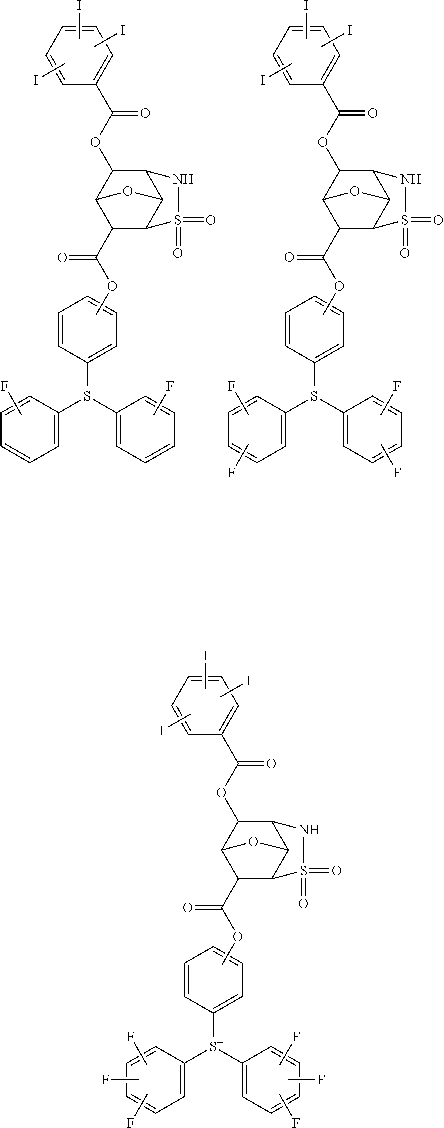

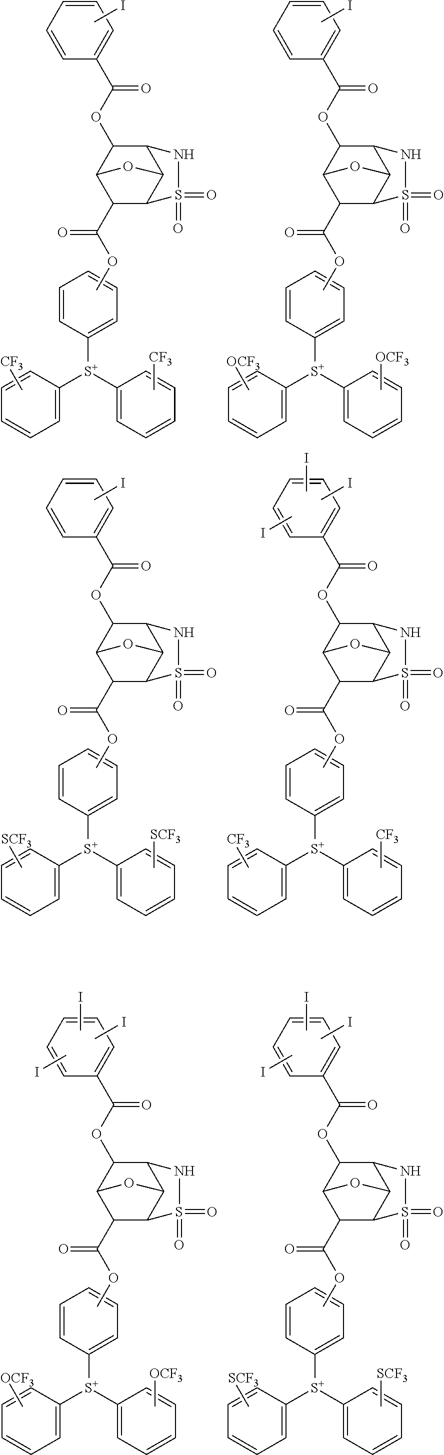

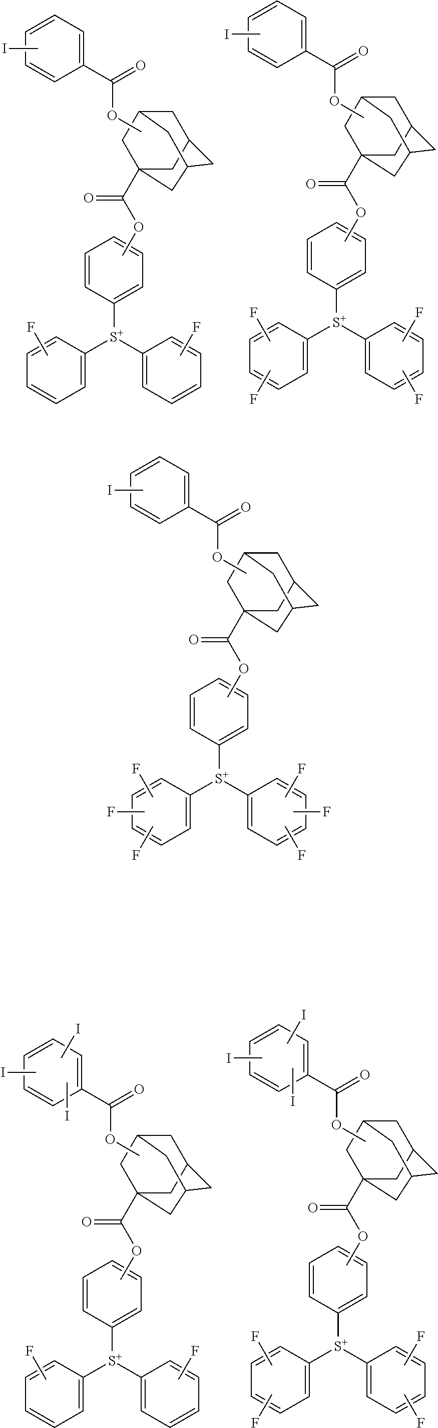

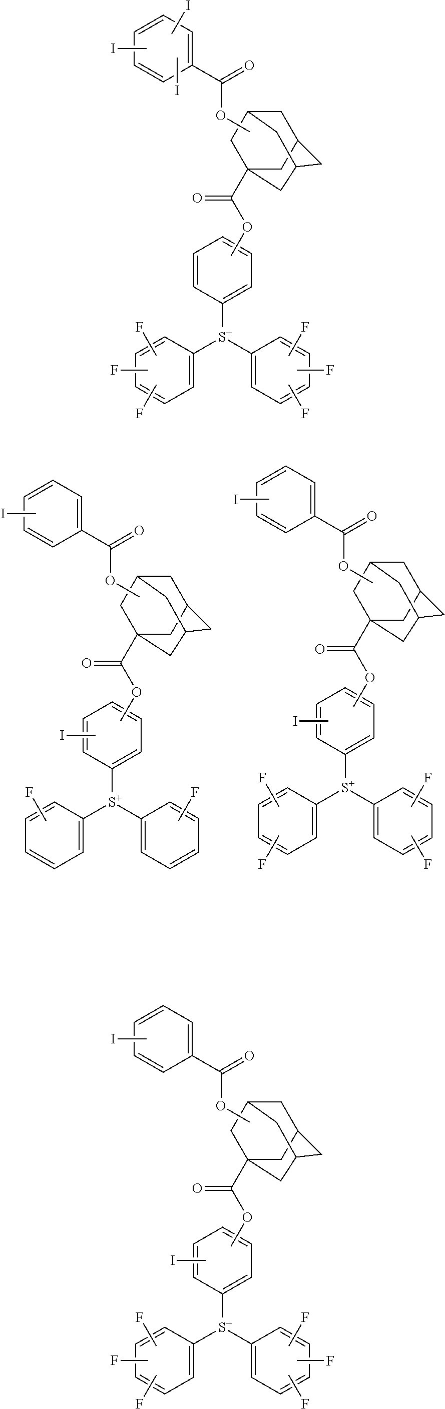

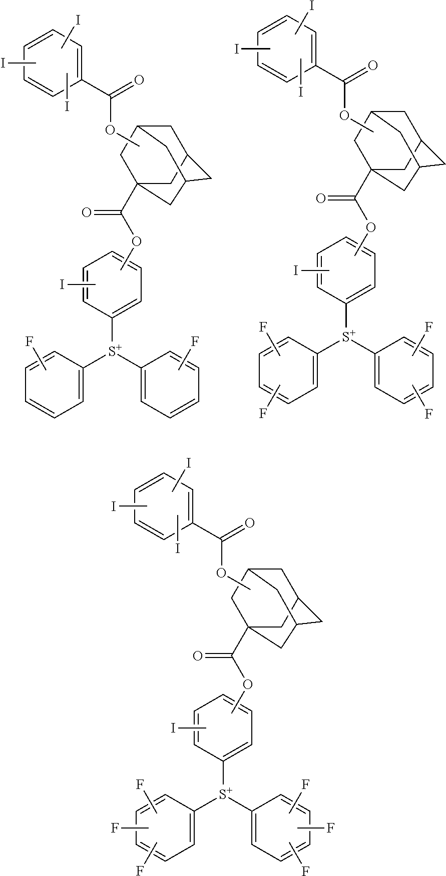

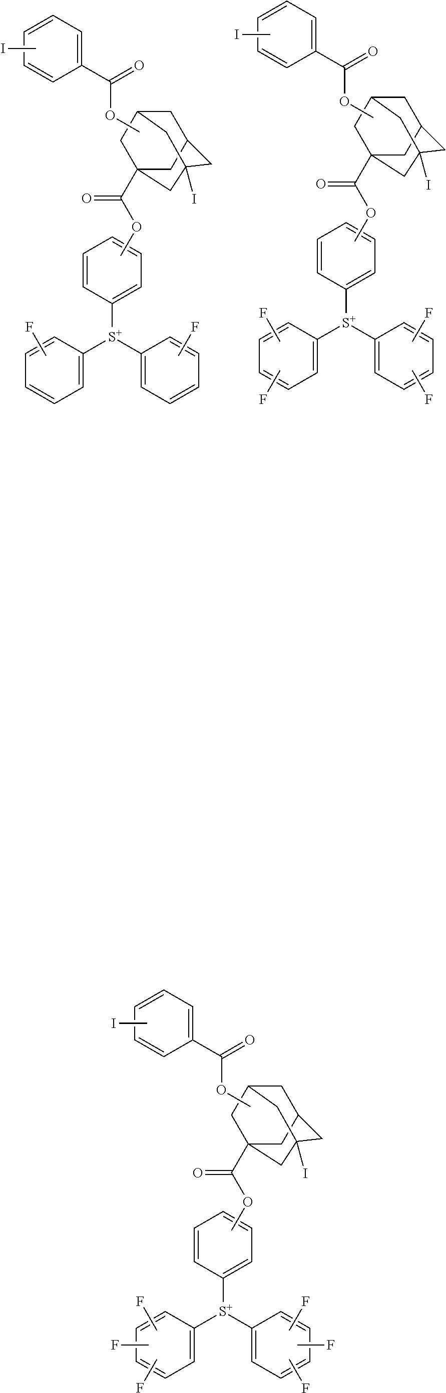

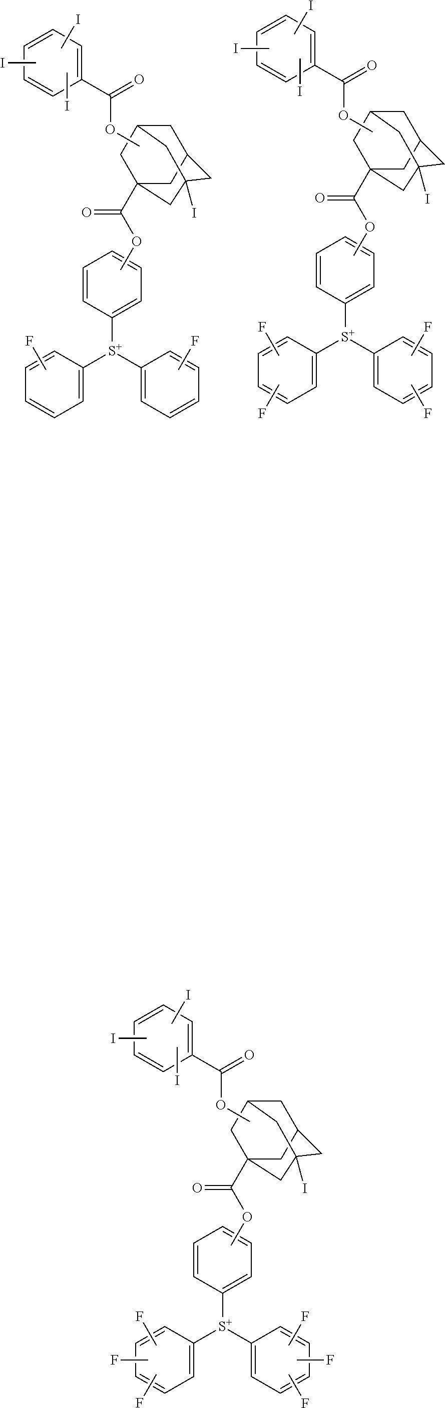

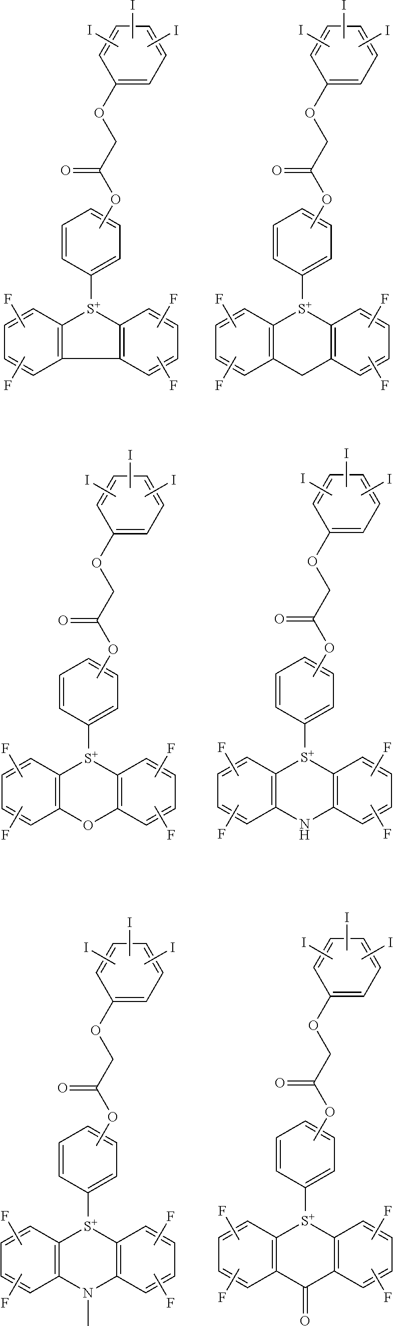

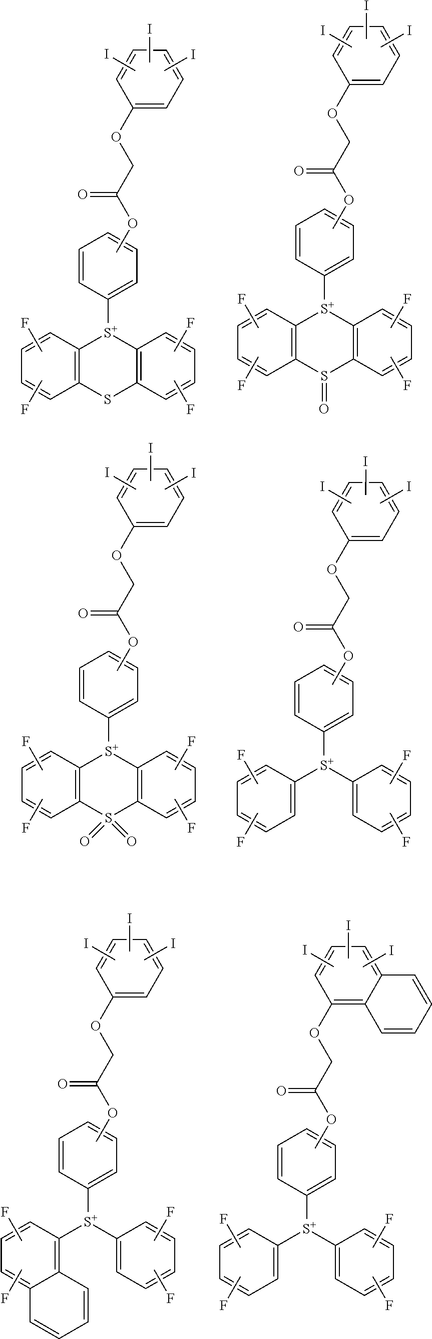

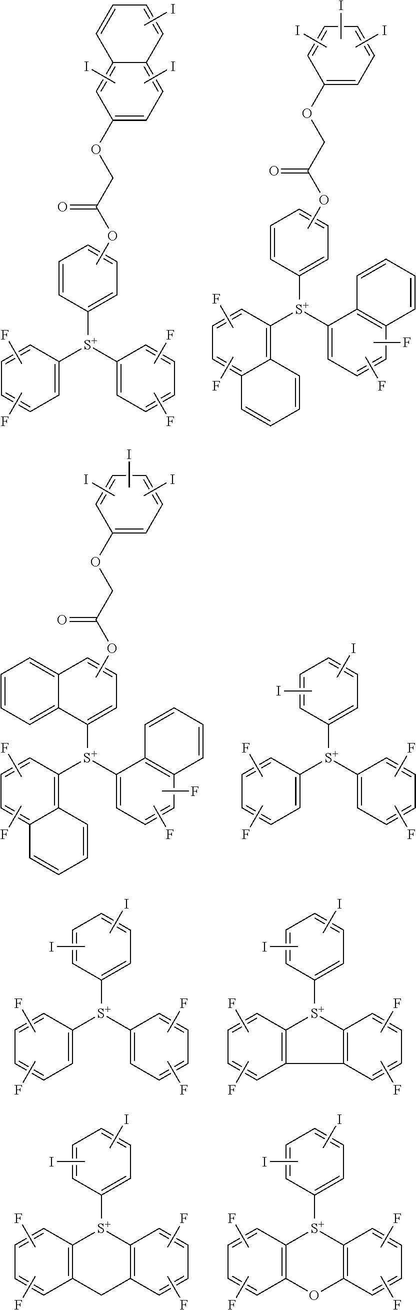

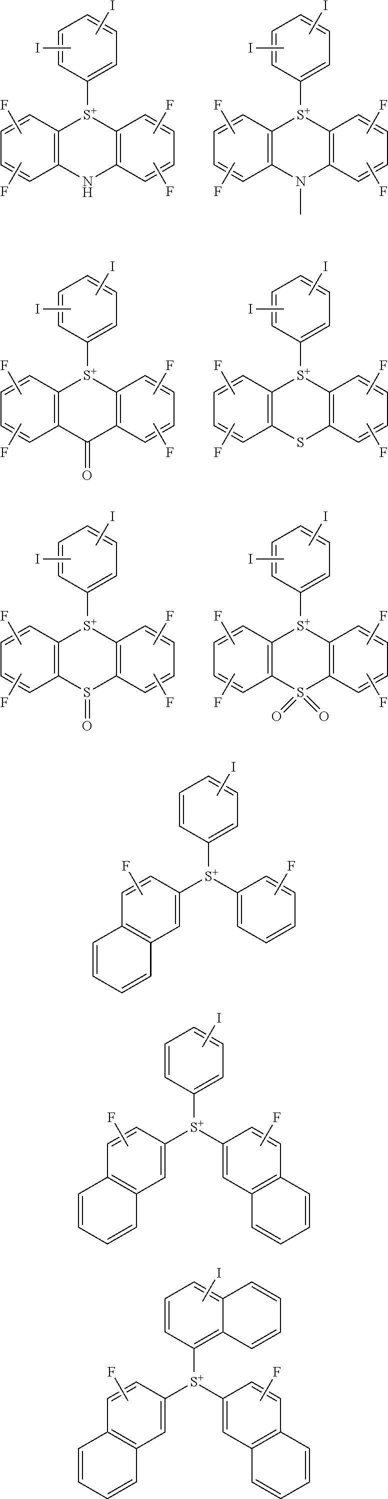

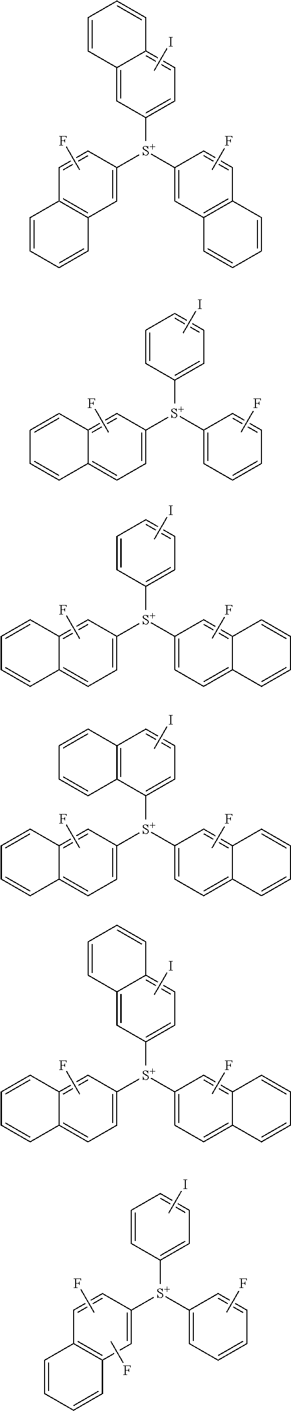

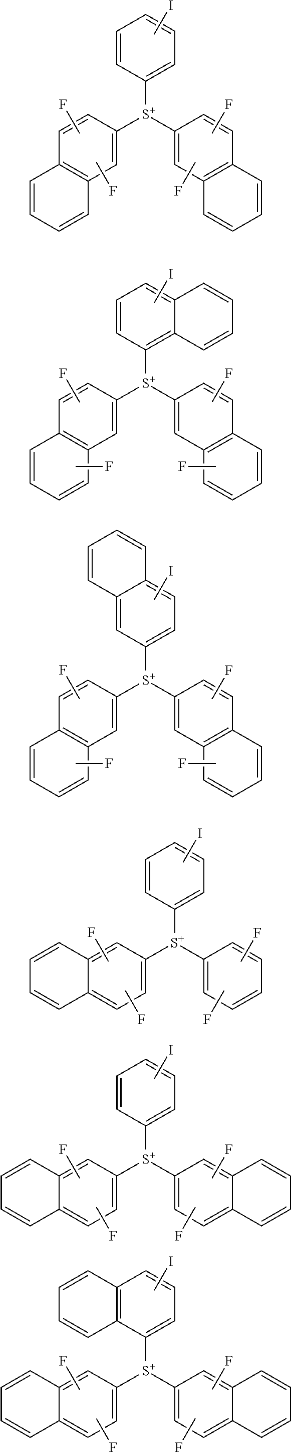

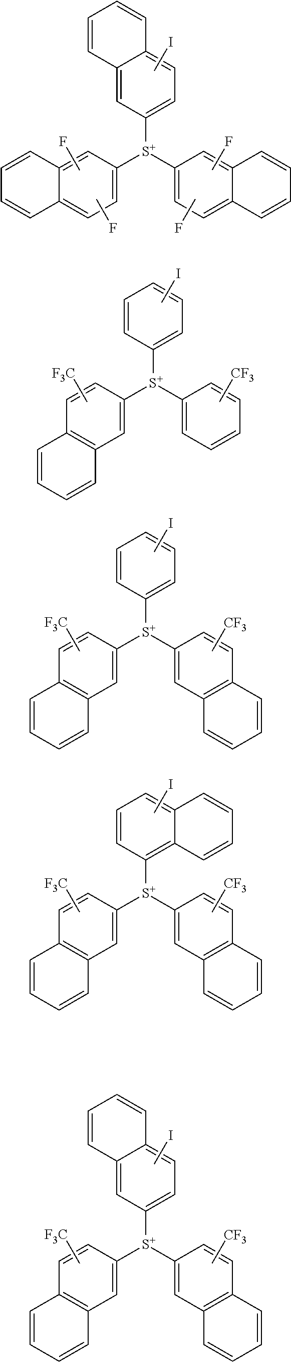

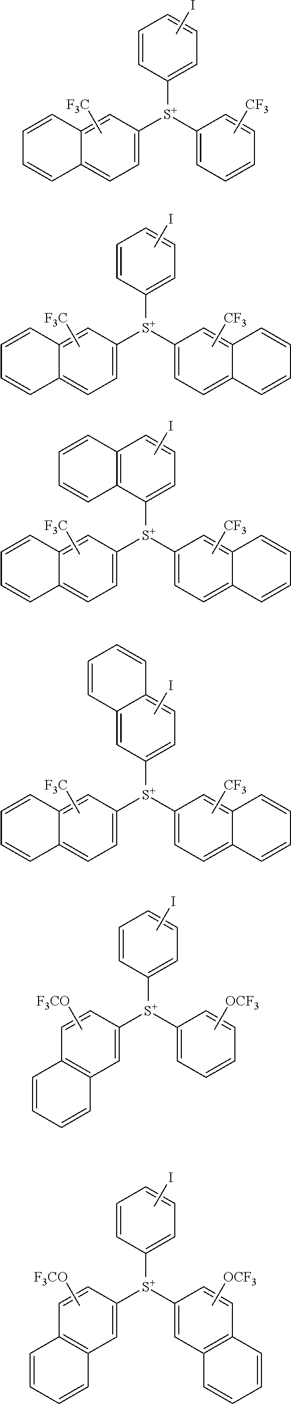

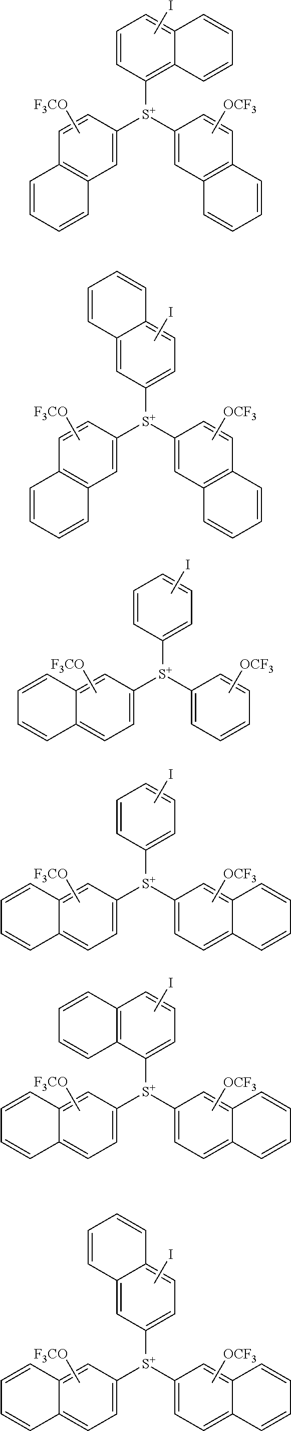

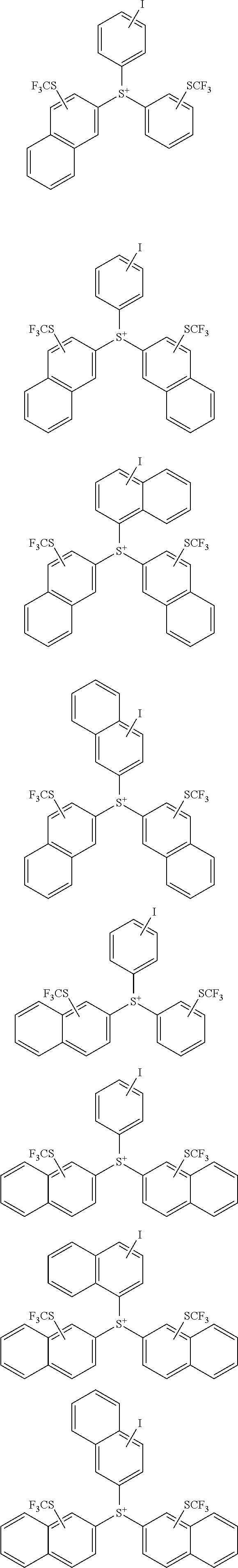

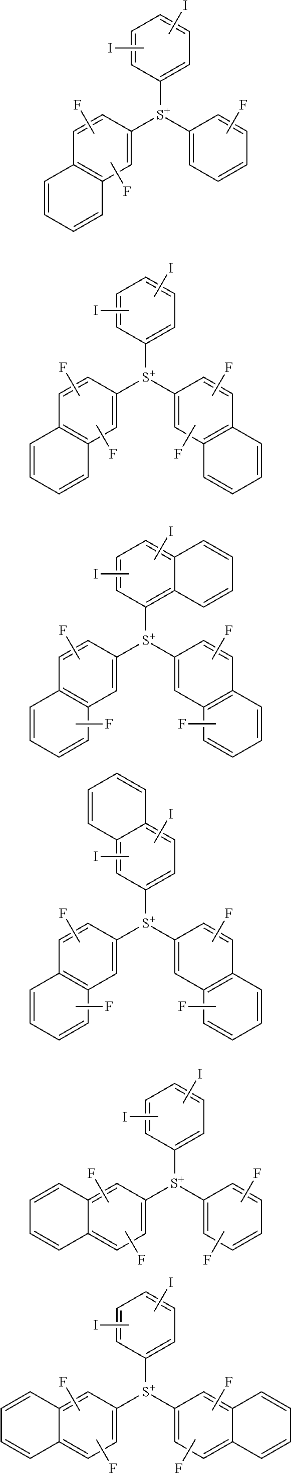

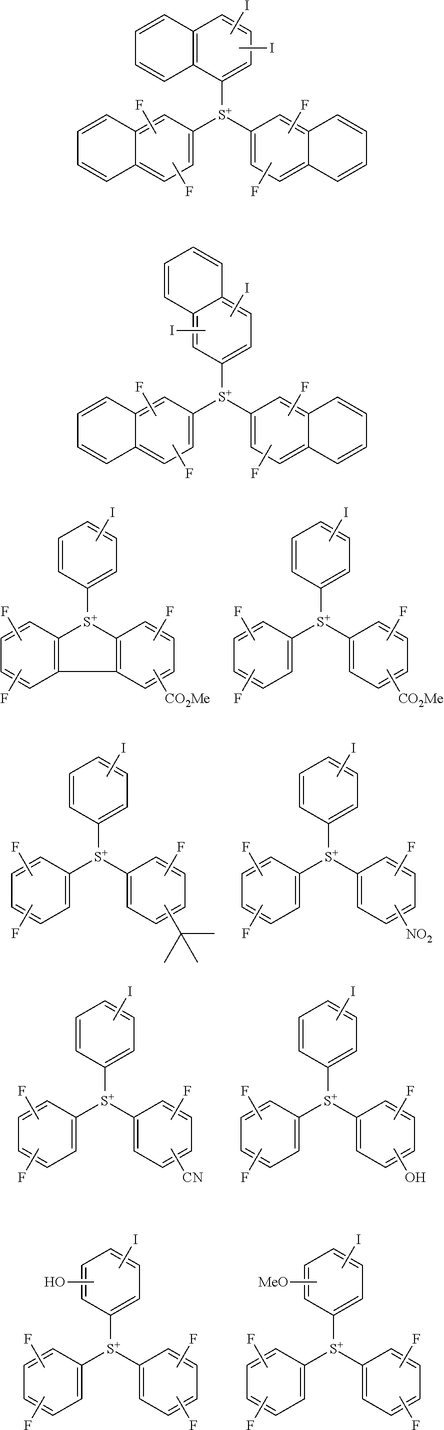

Specific examples of the iodonium cation include those groups described in paragraph of JP-A 2024-259, but not limited thereto.

Specific examples of the ammonium cation include those having the following formula (am-1).

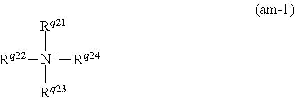

In formula (am-1), R^{q21} to R^{q24} are each independently a C₁-C₄₀ hydrocarbyl group which may contain a heteroatom. R^{q21} and R^{q22} may bond together to form a ring with the nitrogen atom to which they are attached. Specific examples of the hydrocarbyl group are as exemplified above for the hydrocarbyl group represented by R¹ in the description of formula (A).

Specific examples of the ammonium cation having formula (am-1) are shown below, but not limited thereto.

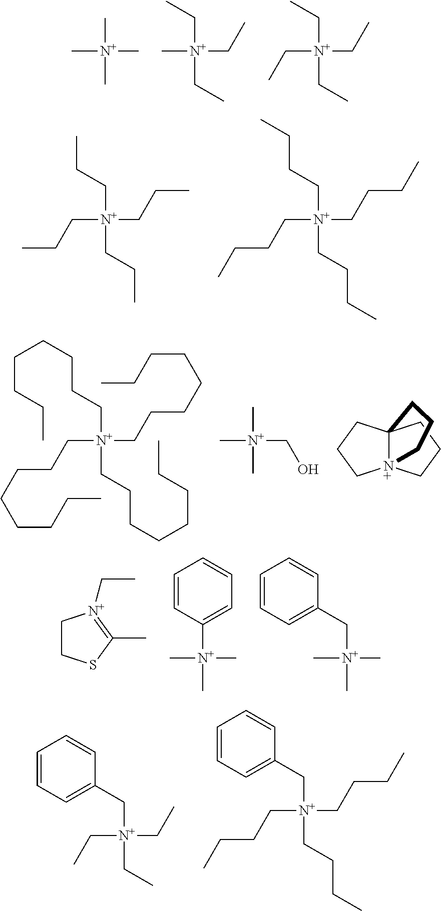

Specific examples of the onium salt having formula (1) or (2) include arbitrary combinations of anions with cations, both as exemplified above. These onium salts are easily prepared by an ion exchange reaction using a known organic chemical method. For the ion exchange reaction, for example, JP-A 2007-145797 can be referred to.

The onium salt having formula (1) or (2) acts as a quencher in the chemically amplified resist composition of the present invention. This is because each counter anion of the onium salt is a conjugate base of a weak acid. The weak acid as used herein means an acid having an acidity at which the acid labile group of the acid labile group-containing unit used for the base polymer cannot be deprotected. The onium salt having formula (1) or (2) functions as a quencher when used in combination with an onium salt type photoacid generator having a conjugate base of a strong acid such as a sulfonic acid whose a-position is fluorinated as a counter anion. That is, in the case of mixing an onium salt generating a strong acid such as a sulfonic acid whose a-position is fluorinated and an onium salt generating a weak acid such as a sulfonic acid or a carboxylic acid that is not fluorinated for use, when the strong acid generated from the photoacid generator by irradiation with high-energy radiation collides with an unreacted onium salt having a weak acid anion, the strong acid is released by salt exchange to produce an onium salt having a strong acid anion. In this process, the strong acid is exchanged to a weak acid having lower catalytic ability, so that the acid is apparently deactivated and acid diffusion can be controlled.

As the quencher (C), the onium salt having a sulfonium cation and a phenoxide anion moiety within one molecule described in JP 6848776, the onium salts having a sulfonium cation and a carboxylate anion moiety within one molecule described in JP 6583136 and JP-A 2020-200311, and the onium salt having an iodonium cation and a carboxylate anion moiety within one molecule described in JP 6274755 can also be used.

When the photoacid generator capable of generating a strong acid is an onium salt, an exchange from the strong acid generated by irradiation with high-energy radiation to a weak acid as described above can take place; however, it is considered that the weak acid generated by irradiation with high-energy radiation collides with the unreacted onium salt generating a strong acid, so that it is difficult to perform a salt exchange. This is due to the phenomenon that the onium cation easily forms an ion pair with an anion of a stronger acid.

When the chemically amplified resist composition of the present invention comprises the onium salt having formula (1) or (2) as the quencher (C), the content thereof is preferably 0.1 to 20 parts by weight and more preferably 0.1 to 10 parts by weight per 80 parts by weight of the base polymer (A). When the content of the onium salt type quencher of the component (C) is in the above range, the resolution is favorable, and the sensitivity is not significantly lowered, which is preferable. The onium salt having formula (1) or (2) may be used alone or in combination of two or more kinds thereof.

The chemically amplified resist composition of the present invention may comprise a nitrogen-containing compound as the quencher (C). Specific examples of the nitrogen-containing compound of the component (C) include primary, secondary, and tertiary amine compounds, specifically amine compounds having a hydroxy group, an ether bond, an ester bond, a lactone ring, a cyano group, or a sulfonic acid ester bond as described in JP-A 2008-111103, paragraphs to [0164]. As in the compound described in JP 3790649, a compound in which a primary or secondary amine is protected with a carbamate group can also be mentioned.

A sulfonium sulfonate having a nitrogen-containing substituent may be used as the nitrogen-containing compound. Such a compound functions as a quencher in the unexposed area, and the exposed area functions as a so-called photodegradable base that loses quencher capability by neutralization with its own generated acid. By using the photodegradable base, the contrast between the exposed area and the unexposed area can be further enhanced. As the photodegradable base, for example, JP-A 2009-109595, JP-A 2012-46501, and the like can be referred to.

When the chemically amplified resist composition of the present invention comprises the nitrogen-containing compound as the quencher (C), the content thereof is preferably 0.001 to 12 parts by weight and more preferably 0.01 to 8 parts by weight per 80 parts by weight of the base polymer (A). The nitrogen-containing compound may be used alone or in combination of two or more kinds thereof.

### Photoacid Generator (D)

The chemically amplified resist composition of the present invention may comprise a photoacid generator as a component (D). The photoacid generator is not particularly limited as long as it is a compound capable of generating an acid by irradiation with high-energy radiation. Examples of suitable photoacid generators include those having the following formula (3) or (4).

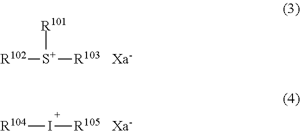

In formula (3), R¹⁰¹ to R¹⁰⁵ are each independently a halogen atom, or a C₁-C₂₀ hydrocarbyl group which may contain a heteroatom. Any two of R¹⁰¹, R¹⁰², and R 103 may bond together to form a ring with the sulfur atom to which they are attached.

Specific examples of the cation of the sulfonium salt having formula (3) include those exemplified as specific examples of the sulfonium cation in formula (A), those described in paragraphs to of JP-A 2024-3744, those described in paragraphs to of WO 2024/128017, those described in paragraphs to of JP 7491173, and those exemplified as specific examples of the sulfonium cation having formula (sulfo-1), but not limited thereto. Specific examples of the cation of the iodonium salt having formula (4) include those groups described in paragraph of JP-A 2024-259, but not limited thereto.

In formulae (3) and (4), Xa is an anion of a strong acid. Examples of the anion of the strong acid include those having any one of the following formulae (Xa-1) to (Xa-4).

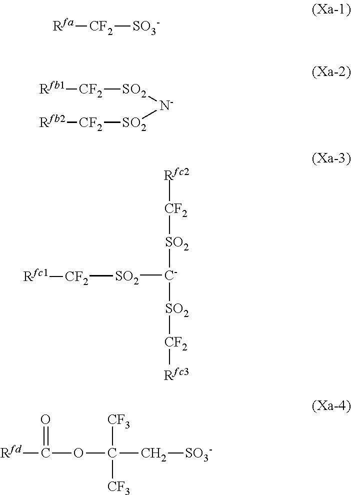

In formula (Xa-1), R^{fa} is a fluorine atom, or a C₁-C₄₀ hydrocarbyl group which may contain a heteroatom. The hydrocarbyl group may be saturated or unsaturated, and may be straight, branched, or cyclic. Specific examples thereof are as exemplified above for the hydrocarbyl group represented by R^{fa1} in formula (Xa-1-1) described below.

The anion having formula (Xa-1) preferably has the following formula (Xa-1-1):

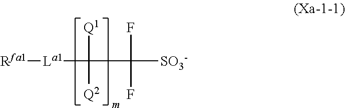

In formula (Xa-1-1), Q¹ and Q² are each independently a hydrogen atom, a fluorine atom, or a C₁-C₆ fluorinated saturated hydrocarbyl group, but at least one of Q¹¹ and Q¹² is preferably a trifluoromethyl group for improving solvent solubility. m is 0, 1, 2, 3, or 4, but is particularly preferably 1. R^{fa1} is a C₁-C₃₅ hydrocarbyl group which may contain a heteroatom. The heteroatom is preferably an oxygen atom, a nitrogen atom, a sulfur atom, a halogen atom, or the like, and more preferably an oxygen atom. The hydrocarbyl group is particularly preferably a C₆-C₃₀ hydrocarbyl group from the viewpoint of obtaining a high resolution in fine pattern formation.

In formula (Xa-1-1), the C₁-C₃₅ hydrocarbyl group represented by R^{fa1} may be saturated or unsaturated and straight, branched, or cyclic. Specific examples thereof include C₁-C₃₅ alkyl groups such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; C₃-C₃₅ cyclic saturated hydrocarbyl groups such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; C₂-C₃₅ unsaturated aliphatic hydrocarbyl groups such as a 2-propenyl group and a 3-cyclohexenyl group; C₆-C₃₅ aryl groups such as a phenyl group, a 1-naphthyl group, a 2-naphthyl group, and a 9-fluorenyl group; C₇-C₃₅ aralkyl groups such as a benzyl group and a diphenylmethyl group; and groups obtained by combining these.

Some or all of hydrogen atoms of the hydrocarbyl group may be substituted with a group containing a heteroatom such as an oxygen atom, a sulfur atom, a nitrogen atom, or a halogen atom, and some of—CH₂-of the hydrocarbyl group may be substituted with a group containing a heteroatom such as an oxygen atom, a sulfur atom, or a nitrogen atom, so that the group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), or a haloalkyl group. Specific examples of the hydrocarbyl group containing a heteroatom include a tetrahydrofuryl group, a methoxymethyl group, an ethoxymethyl group, a methylthiomethyl group, an acetamidemethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group. In formula (Xa-1-1), Lal is a single bond, an ether bond, an ester bond, a sulfonic acid ester bond, a carbonate bond, or a carbamate bond, and from the viewpoint of synthesis, is preferably an ether bond or an ester bond and more preferably an ester bond.

Specific examples of the anion having formula (Xa-1) are shown below, but not limited thereto. In the following formula, Q¹ is as defined above, and Ac is an acetyl group.

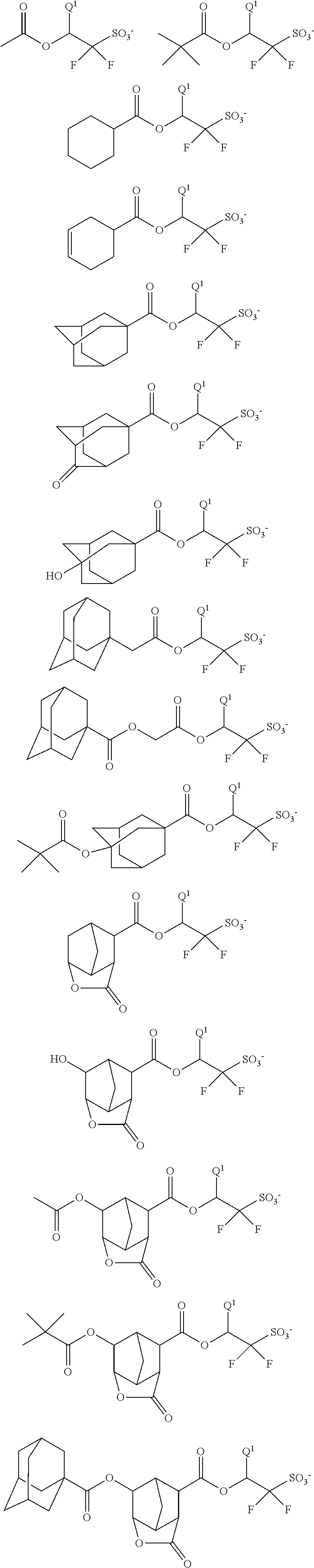

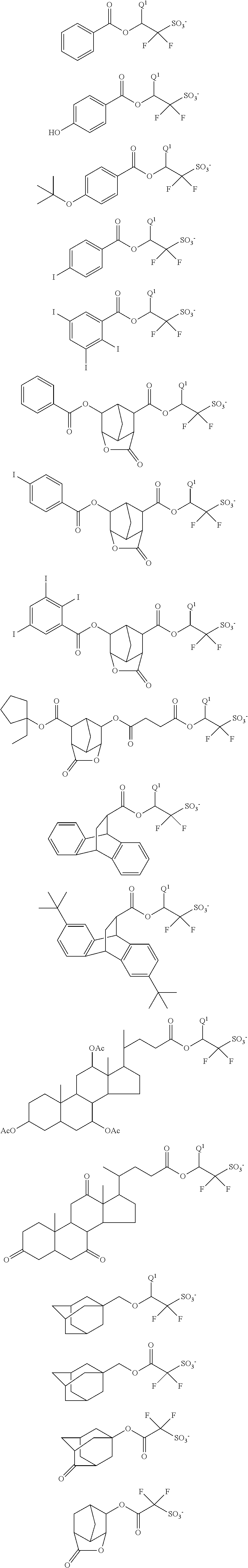

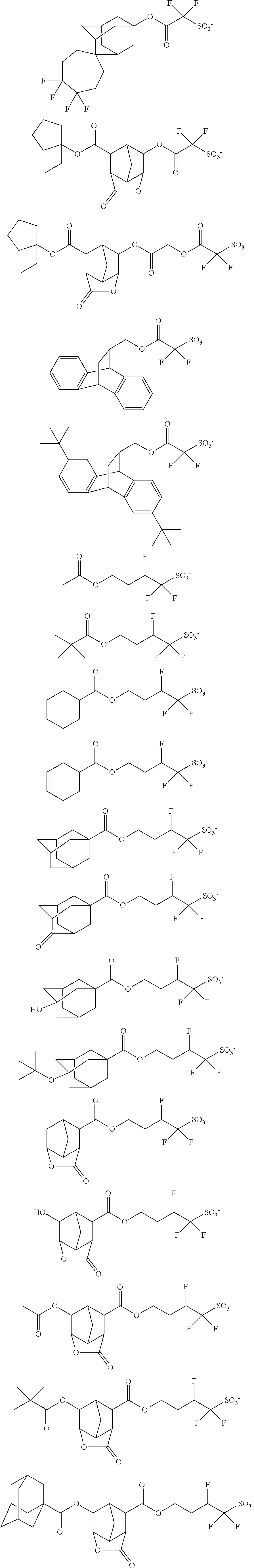

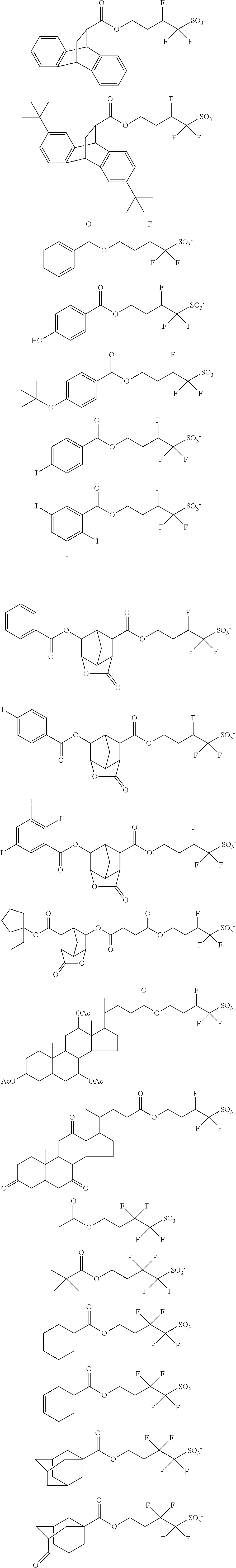

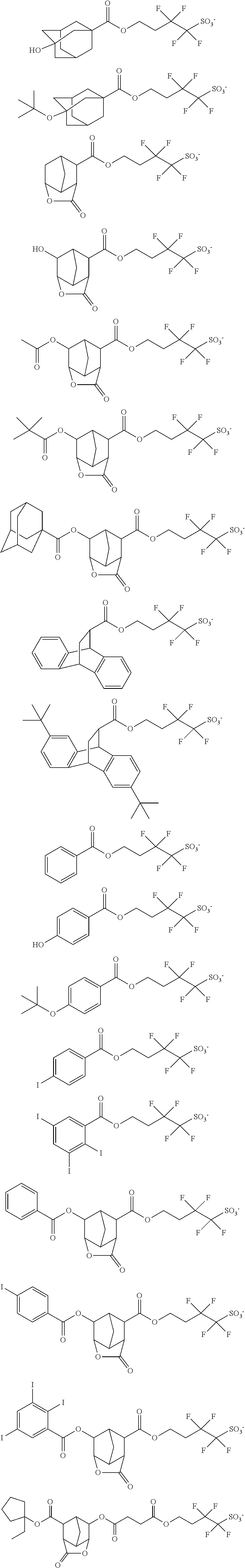

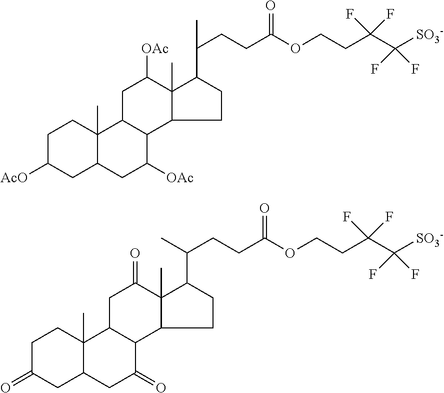

In formula (Xa-2), R^{fb1} and R^{fb2} are each independently a fluorine atom, or a C₁-C₄₀ hydrocarbyl group which may contain a heteroatom. The hydrocarbyl group may be saturated or unsaturated, and may be straight, branched, or cyclic. Specific examples thereof are as exemplified above for the hydrocarbyl group represented by R^{fa1} in formula (Xa-1-1). R^{fb1} and R^{fb2} are preferably a fluorine atom or C₁-C₄ straight fluorinated alkyl group. R^{fb1} and R^{fb2} may bond together to form a ring with the group (—CF₂—SO₂—N—SO₂—CF₂—) to which they are attached, and in this case, the group obtained by bonding R^{fb1} and R^{fb2} to each other is preferably a fluorinated ethylene group or a fluorinated propylene group.

In formula (Xa-3), R^{fc1}, R^{fc2}, and R^{fc3} are each independently a fluorine atom, or a C₁-C₄₀ hydrocarbyl group which may contain a heteroatom. The hydrocarbyl group may be saturated or unsaturated, and may be straight, branched, or cyclic. Specific examples thereof are as exemplified above for the hydrocarbyl group represented by R^{fa1} in formula (Xa-1-1). R^{fc1}, R^{fc2}, and R^{fc3} are preferably a fluorine atom or a C₁-C₄ straight fluorinated alkyl group. R^{fe1} and R^{fe2} may bond together to form a ring with the group (—CF₂—SO₂—C—SO₂—CF₂—) to which they are attached, and in this case, the group obtained by bonding R^{fe1} and R^{fc2} to each other is preferably a fluorinated ethylene group or a fluorinated propylene group.

In formula (Xa-4), R^{fd} is a C₁-C₄₀ hydrocarbyl group which may contain a heteroatom. The hydrocarbyl group may be saturated or unsaturated, and may be straight, branched, or cyclic. Specific examples thereof are as exemplified above for the hydrocarbyl group represented by R^{fa1} in formula (Xa-1-1). 10

Specific examples of the anion having formula (Xa-4) are shown below, but not limited thereto.

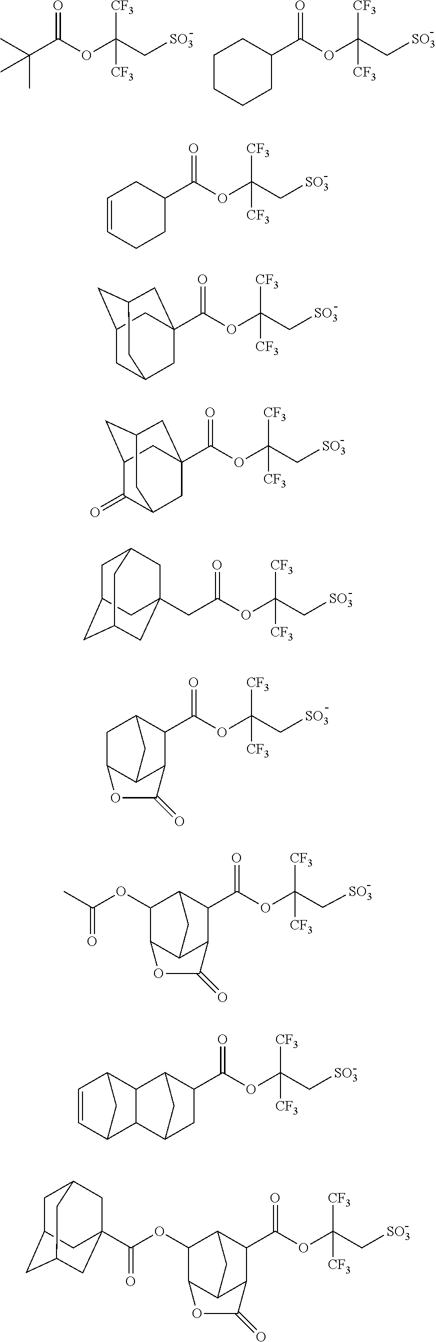

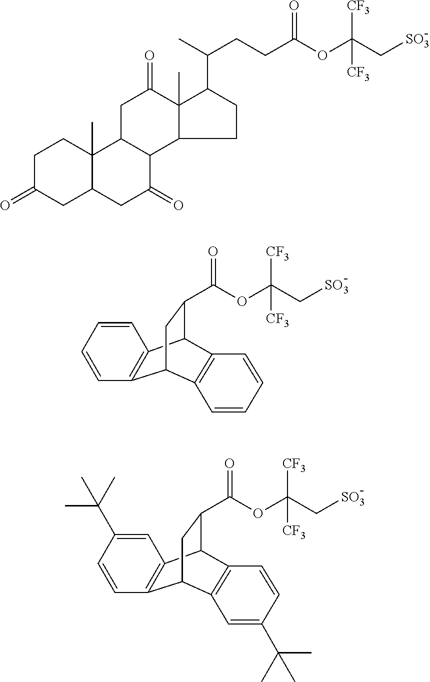

Examples of the non-nucleophilic counter ion further include an anion having an aromatic ring substituted with an iodine atom or a bromine atom. Specific examples of such an anion include those having the following formula (Xa-5).

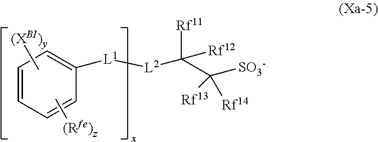

In formula (Xa-5), x is 1, 2, or 3. y is 1, 2, 3, 4, or 5.

z is 0, 1, 2, or 3. Provided that 1≤y+z≤5. y is preferably 1, 2, or 3 and more preferably 2 or 3. z is preferably 0, 1, or 2.

In formula (Xa-5), XBI is an iodine atom or a bromine atom, and may be the same or different when x and/or y is 2 or more.

In formula (Xa-5), L′ is a single bond, an ether bond, an ester bond, or a C₁-C₆ saturated hydrocarbylene group which may contain an ether bond or an ester bond. The saturated hydrocarbylene group may be straight, branched, or cyclic.

In formula (Xa-5), L2 is a single bond or a C₁-C₂₀ divalent linking group when x is 1, and a C₁-C₂₀ (x+1)-valent linking group which may contain an oxygen atom, a sulfur atom, or a nitrogen atom when x is 2 or 3.

In formula (Xa-5), R^{fe} is a hydroxy group, a carboxy group, a fluorine atom, a chlorine atom, a bromine atom, an amino group, a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain a fluorine atom, a chlorine atom, a bromine atom, a hydroxy group, an amino group, or an ether bond, or —N(R^{feA})(R^{feB}), —N(R^{feC})—C(═O)—R^{feD}, or —N(R^{feC})—C(═O)—O—R^{feD}. R^{feA} and R^{feB} are each independently a hydrogen atom or a C₁-C₆ saturated hydrocarbyl group. R^{feC} is a hydrogen atom or a C₁-C₆ saturated hydrocarbyl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-Cis aralkyl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched, or cyclic. The hydrocarbyl group, the hydrocarbyloxy group, the hydrocarbylcarbonyl group, the hydrocarbyloxycarbonyl group, the hydrocarbylcarbonyloxy group, and hydrocarbylsulfonyloxy group may be straight, branched, or cyclic. When x and/or z is 2 or more, respective R^{fe} may be the same as or different from each other.

Of these, R^{fe} is preferably a hydroxy group, —N(R^{feC})—C(═O)—R^{feD}, —N(R^{feC})—C(═O)—O—R^{feD}, a fluorine atom, a chlorine atom, a bromine atom, a methyl group, a methoxy group, or the like.

In formula (Xa-5), R^{f11} to R^{f14} are each independently a hydrogen atom, a fluorine atom, or a trifluoromethyl group, and at least one of R^{f11} to R^{f14} is a fluorine atom or a trifluoromethyl group. R^{f11} and R^{f12}, taken together, may form a carbonyl group. Particularly, both R^{f13} and R^{f14} are preferably a fluorine atom.

Specific examples of the anion having formula (Xa-5) are shown below, but not limited thereto. In the formulae. X^{BI} is as defined above.

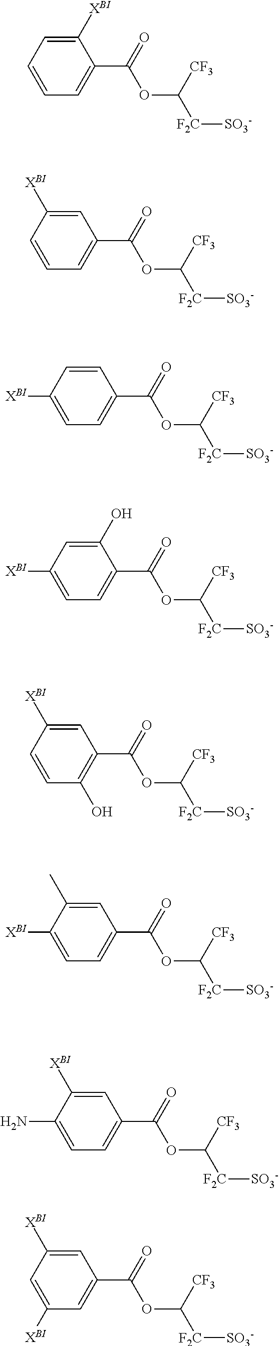

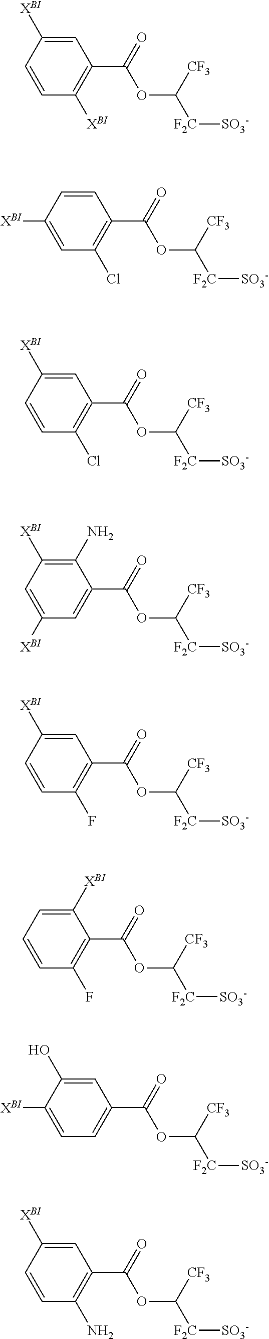

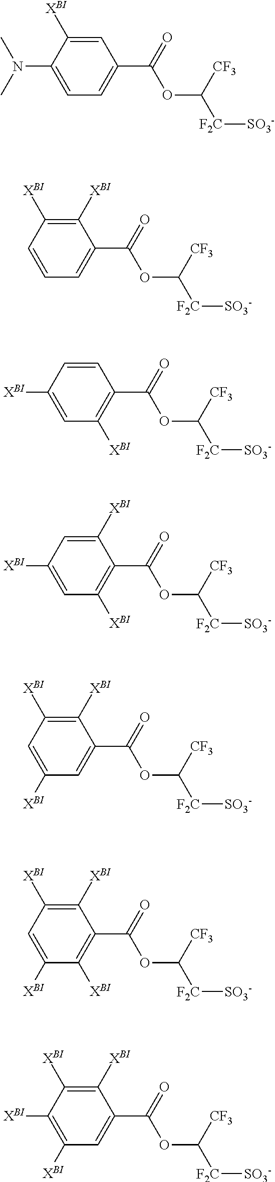

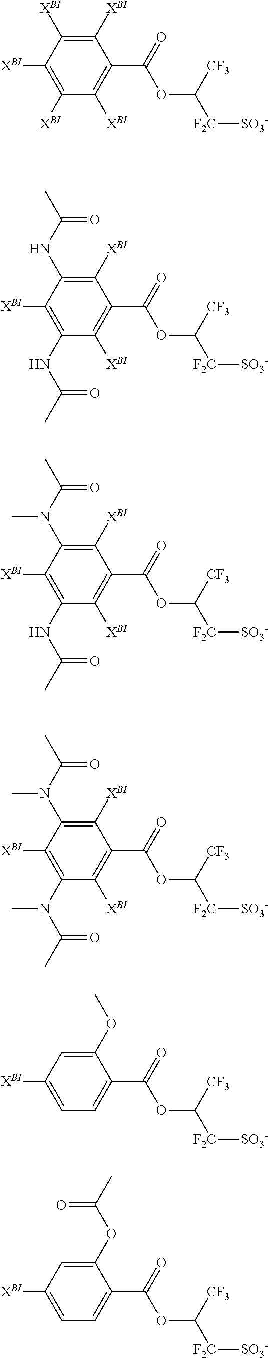

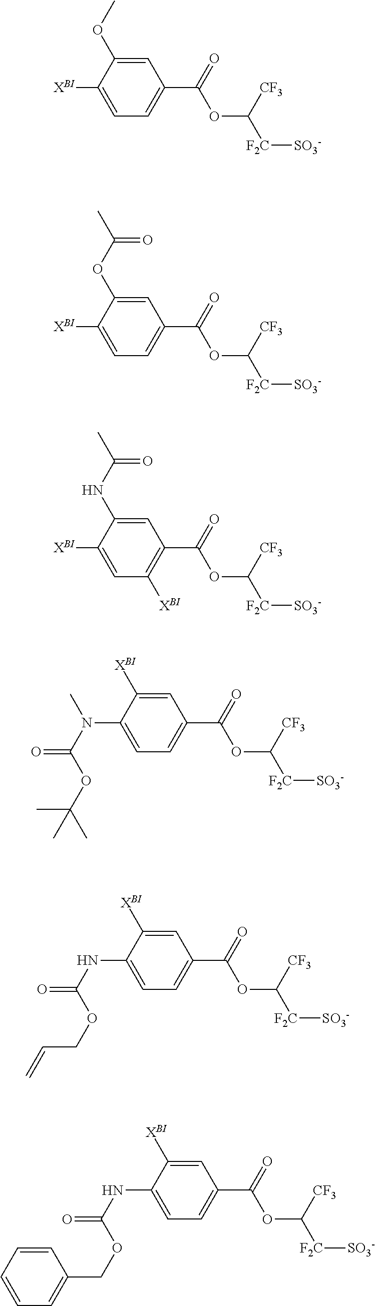

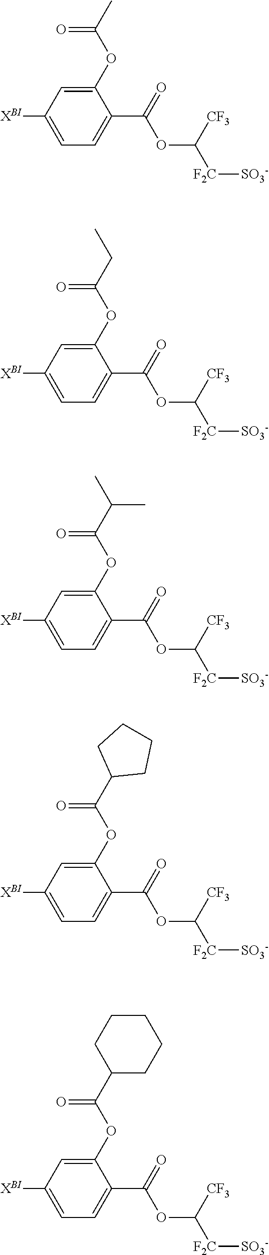

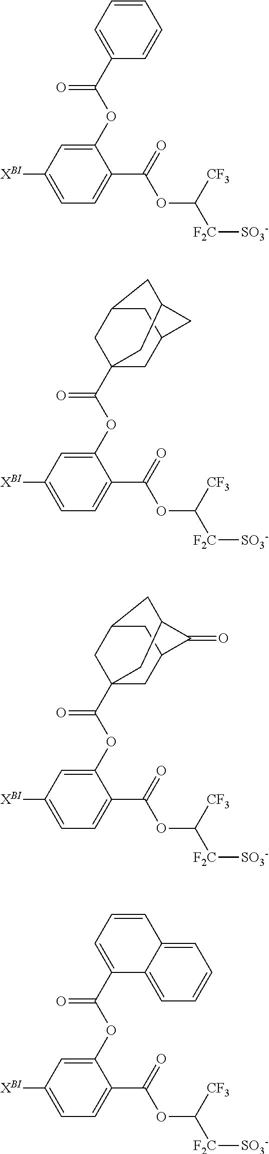

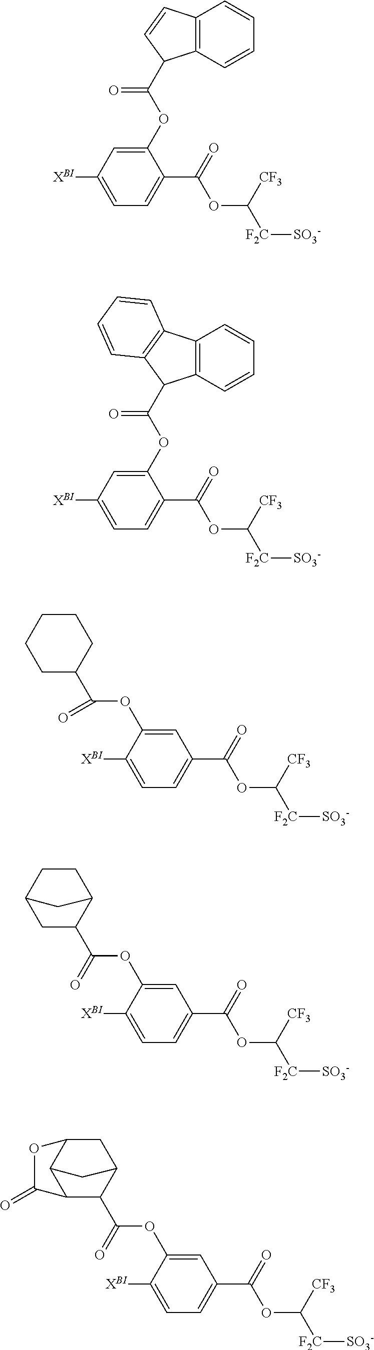

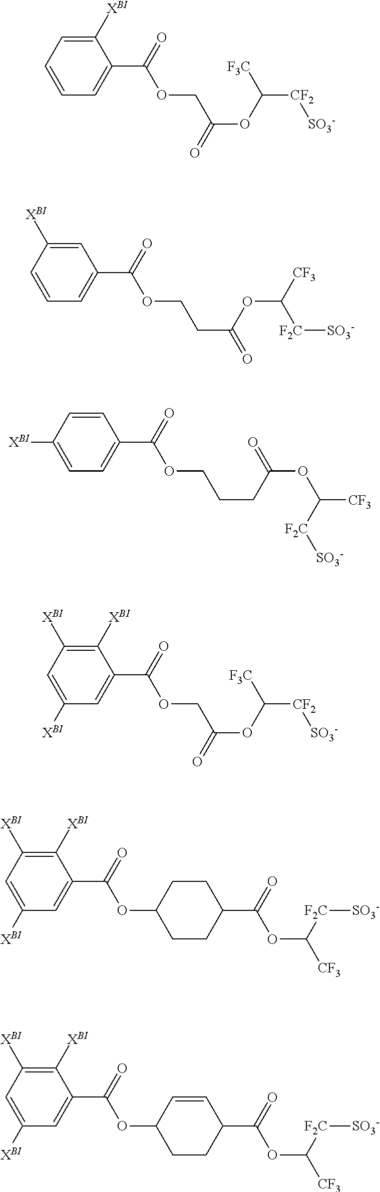

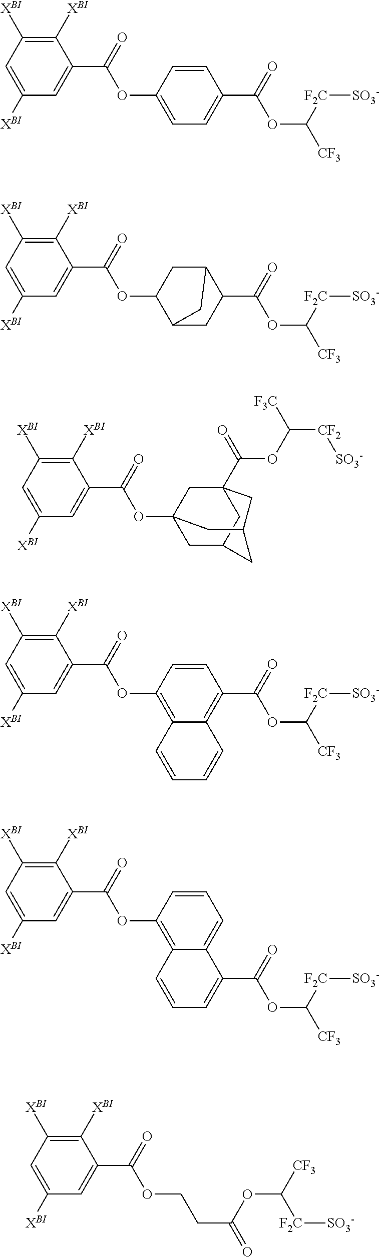

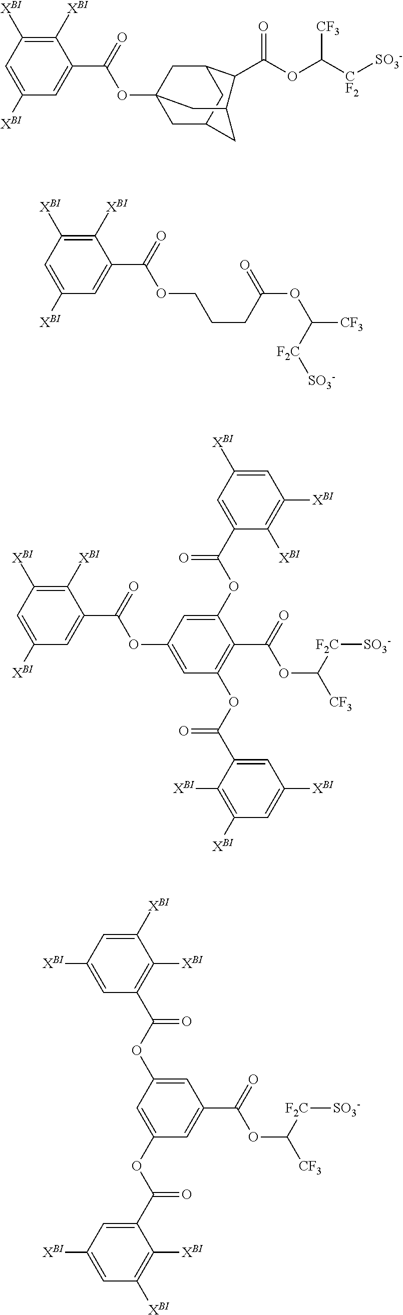

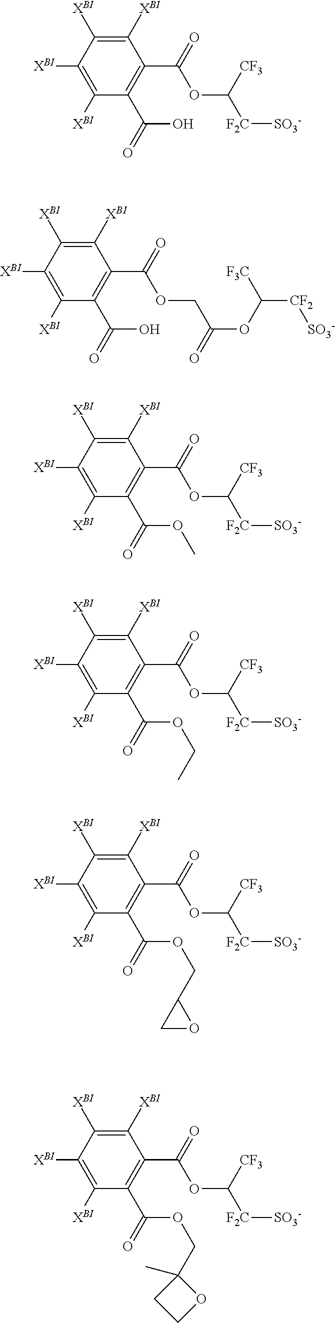

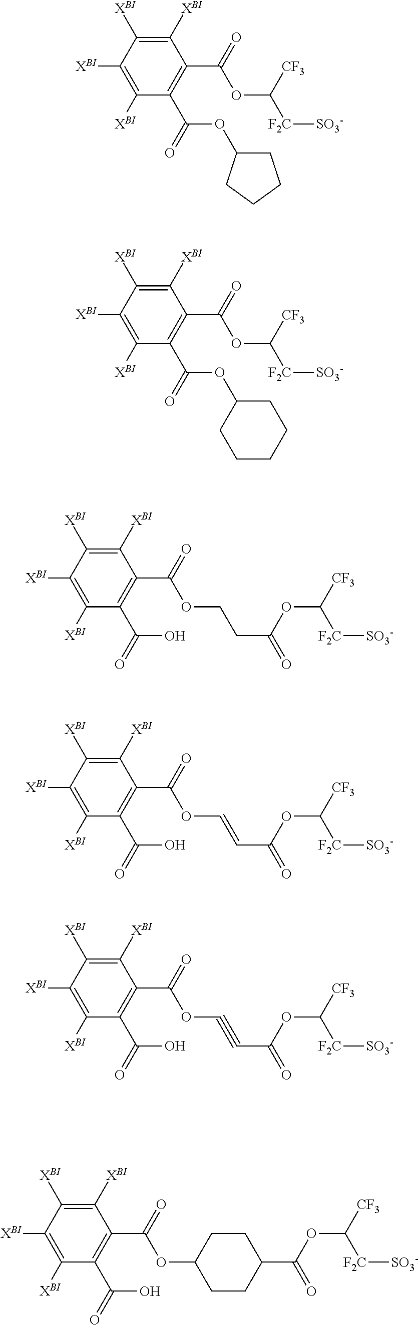

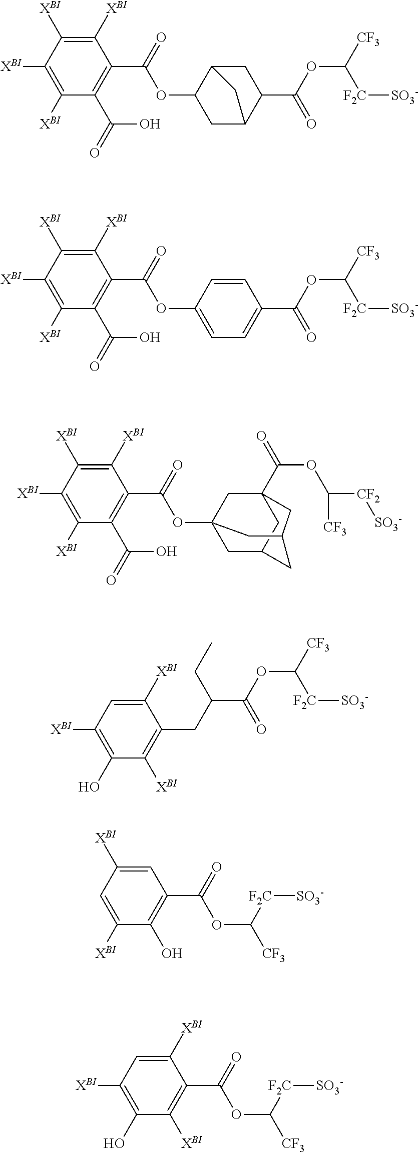

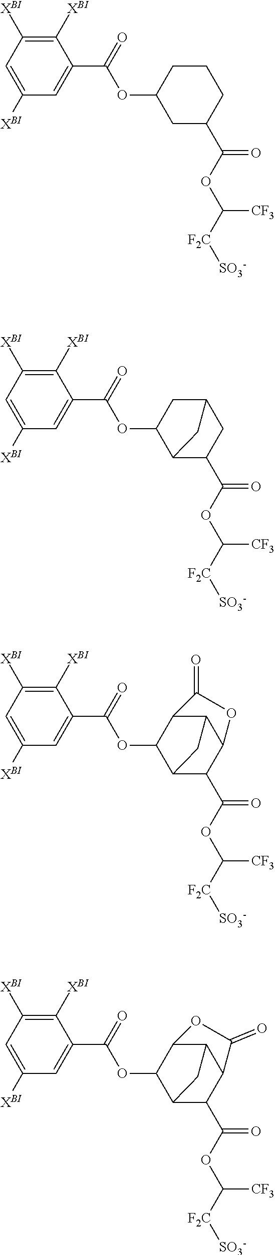

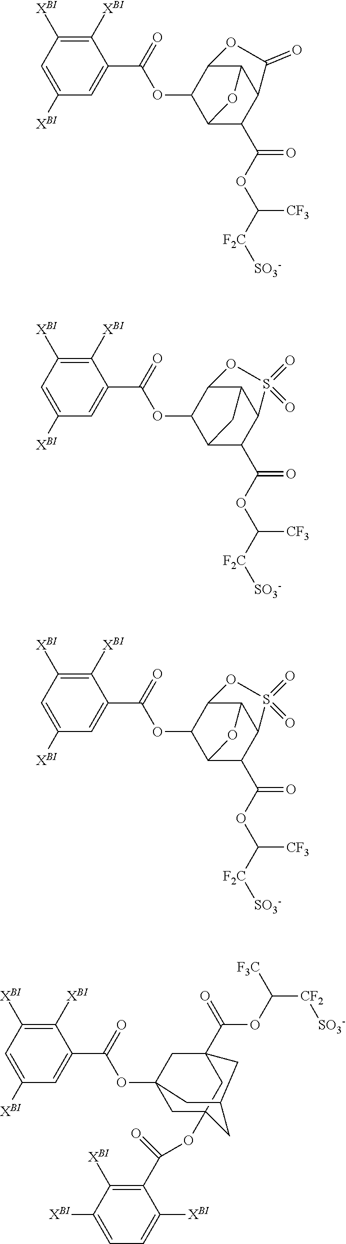

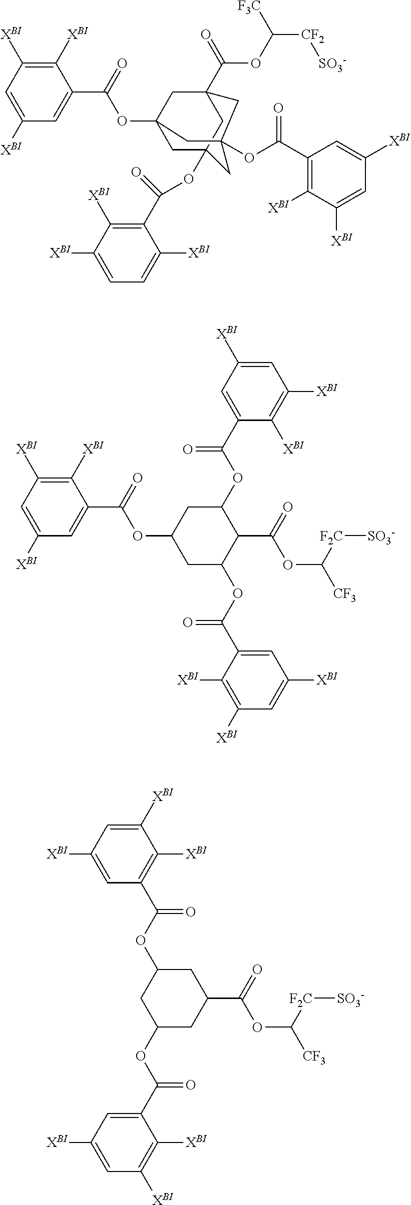

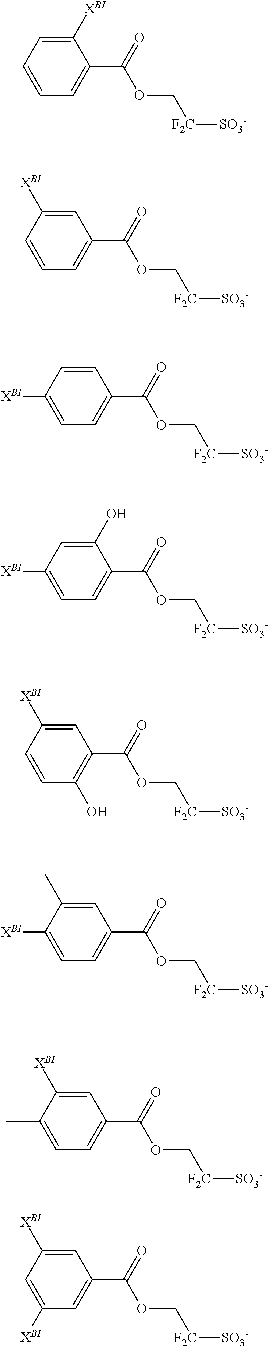

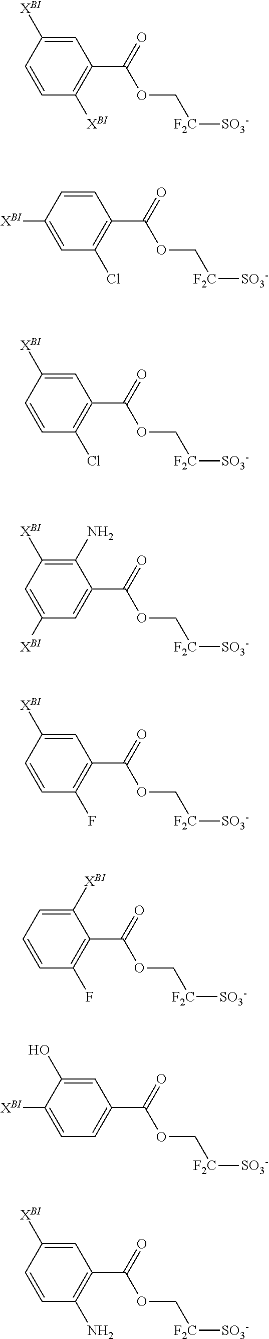

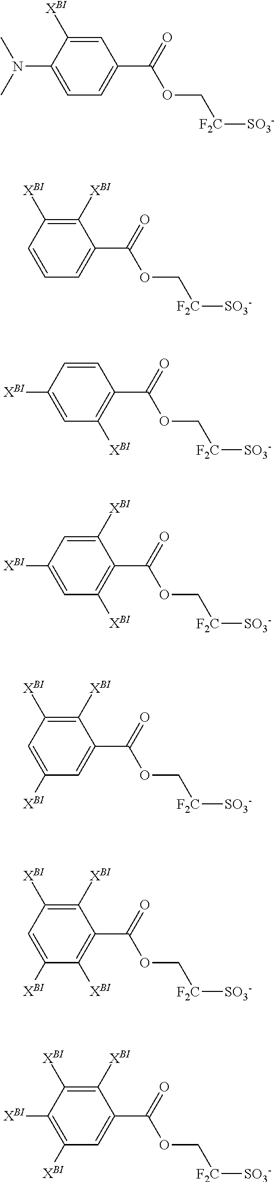

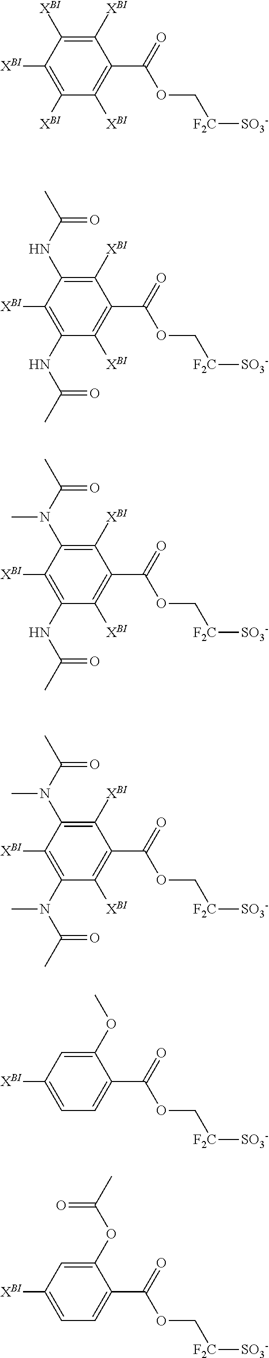

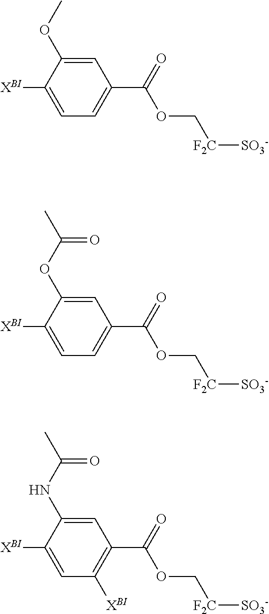

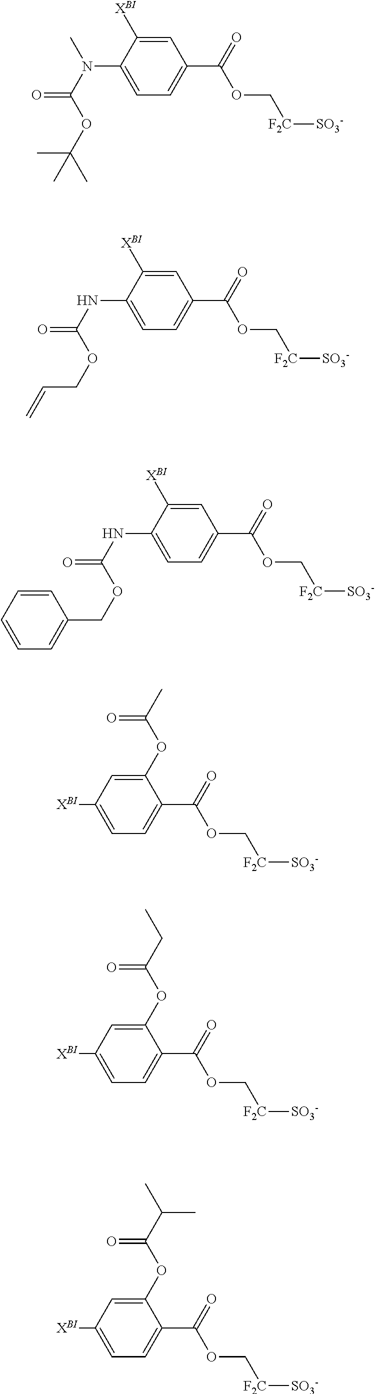

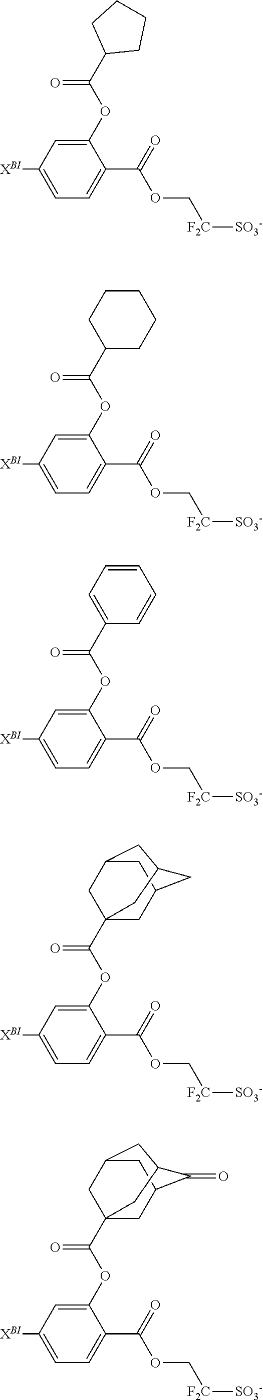

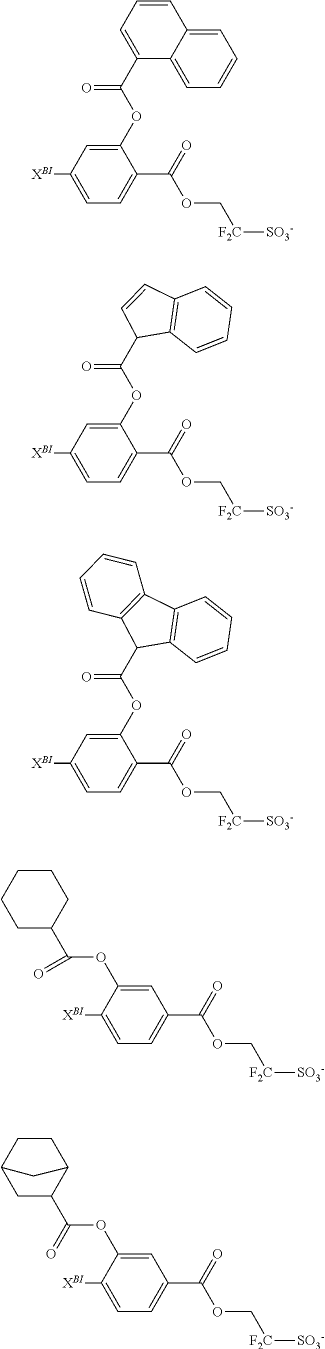

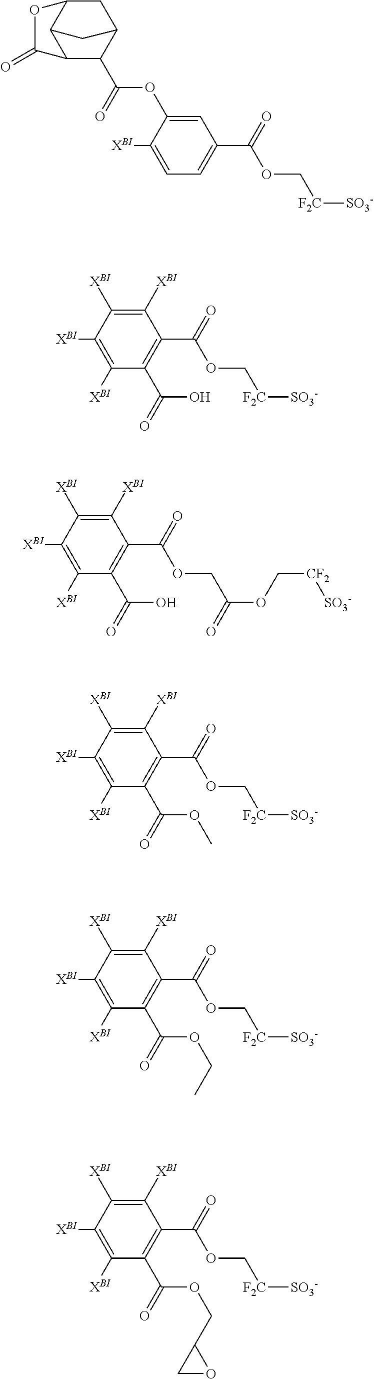

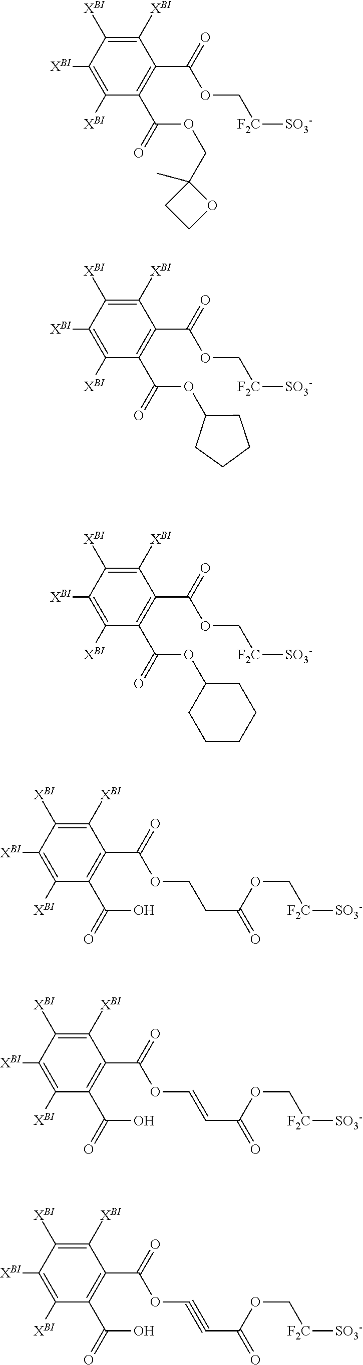

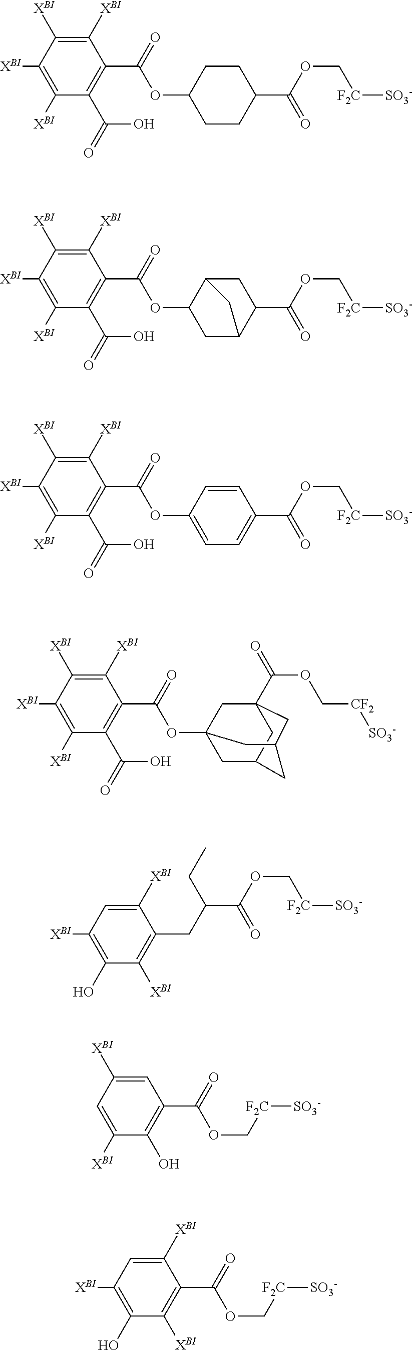

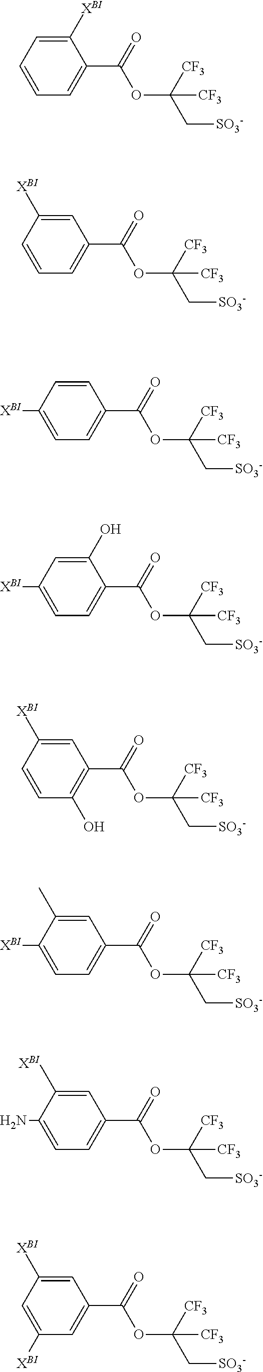

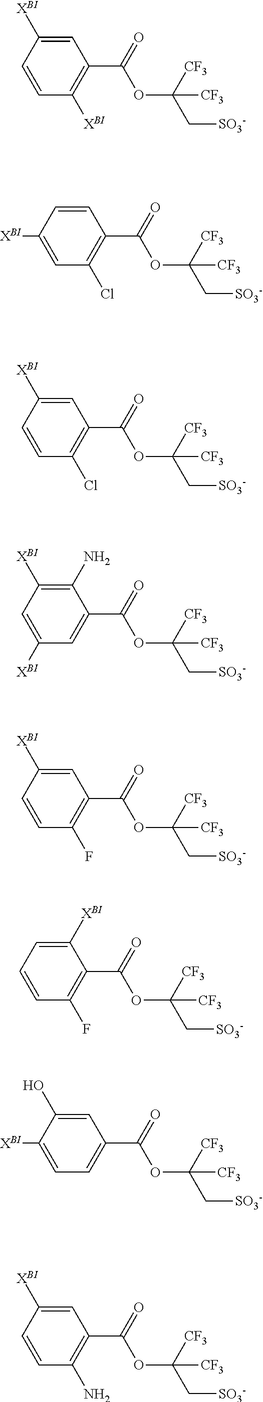

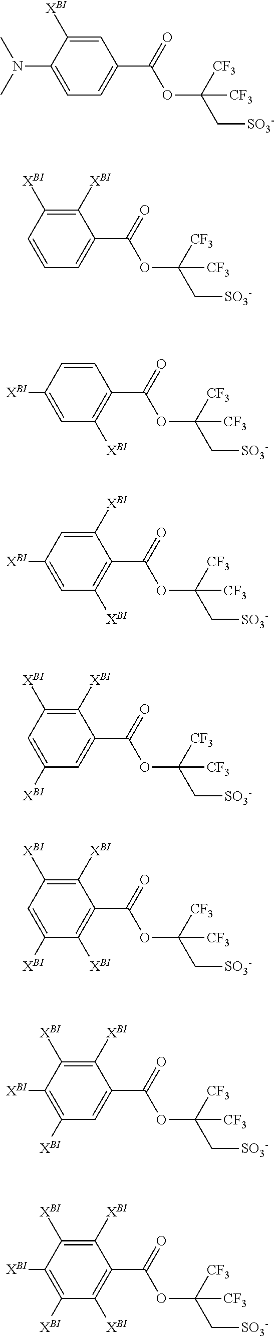

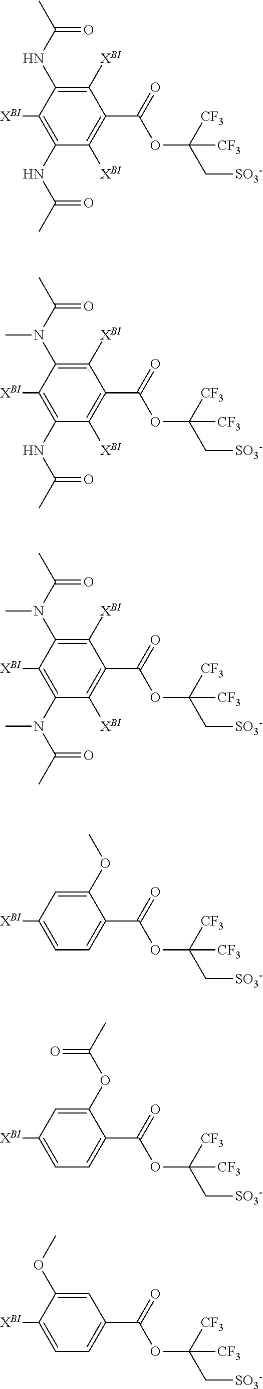

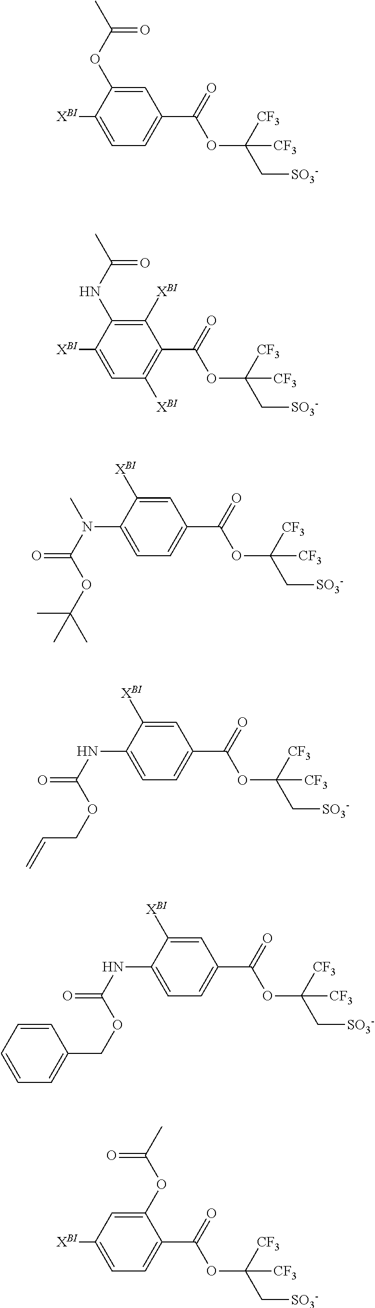

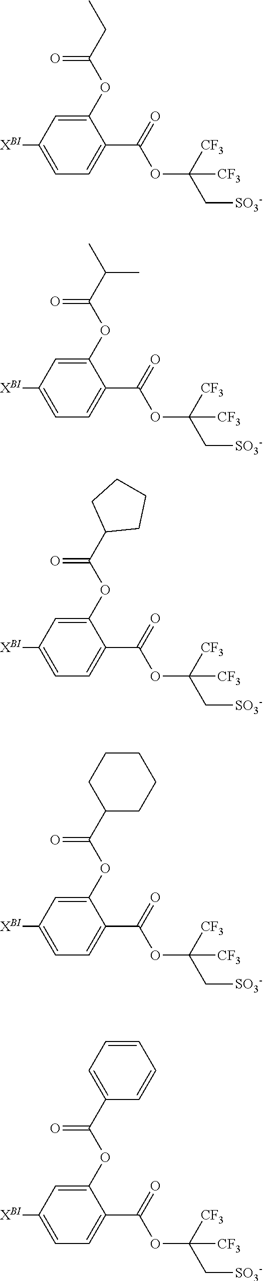

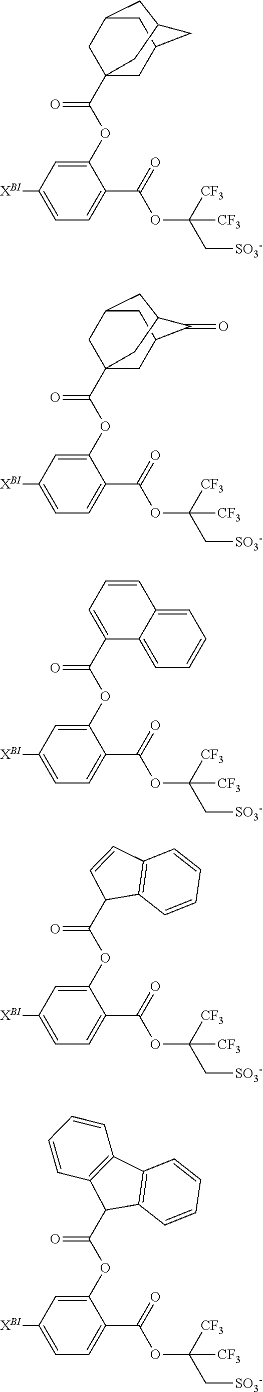

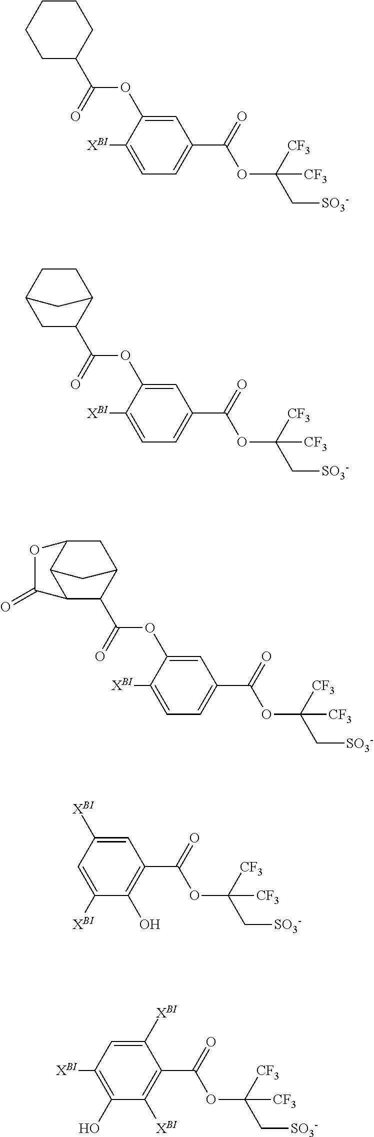

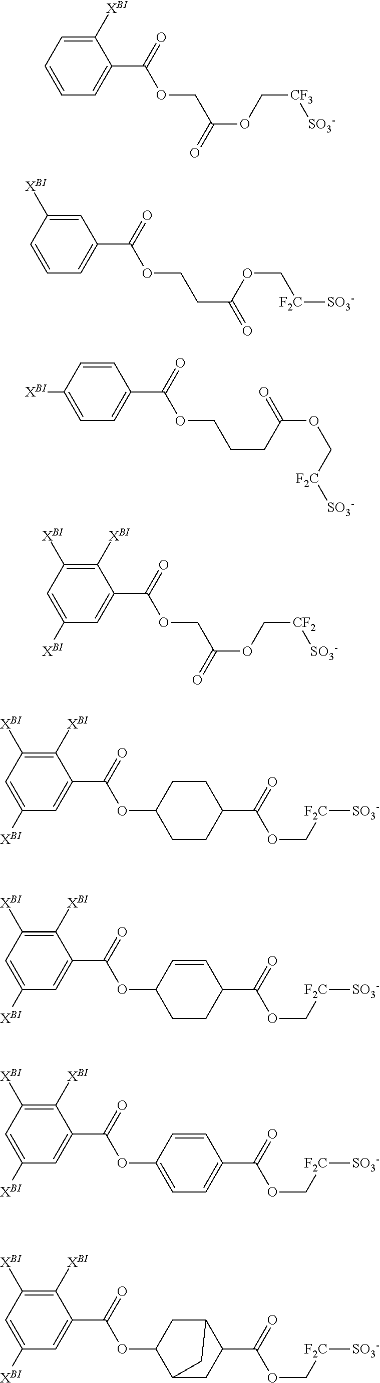

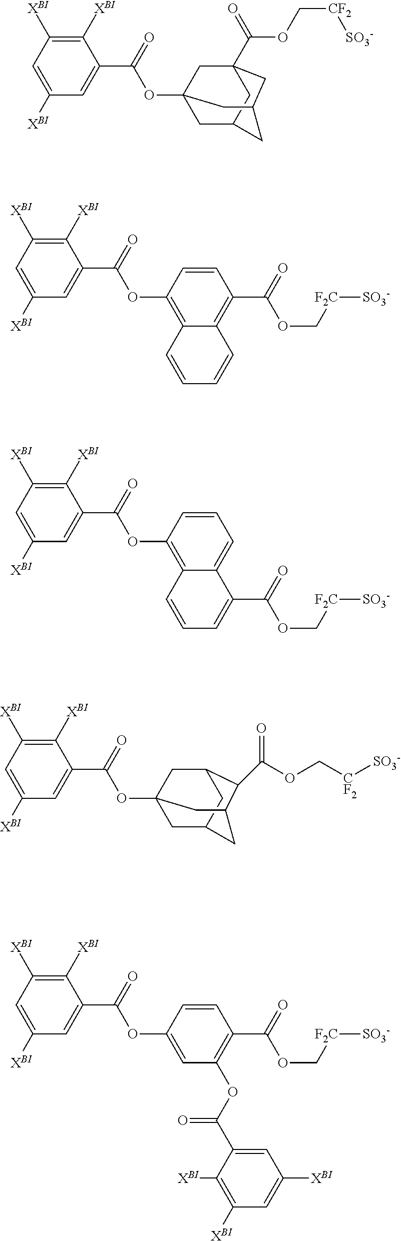

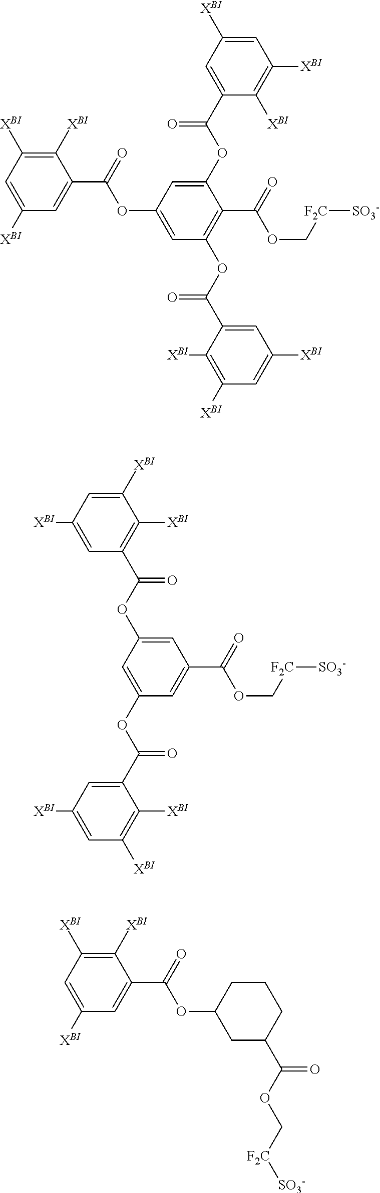

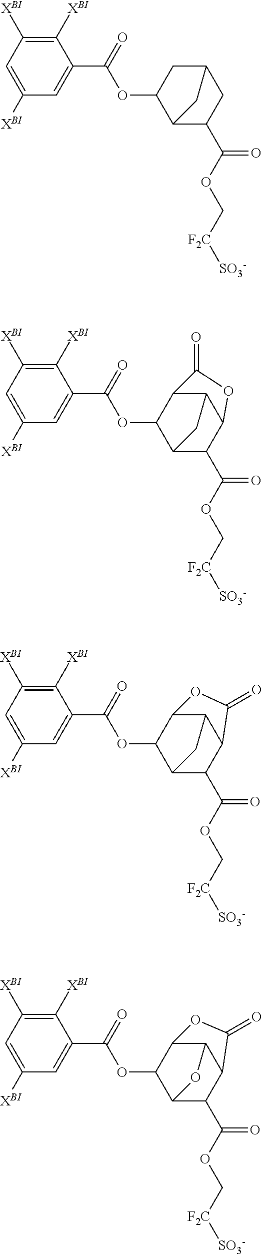

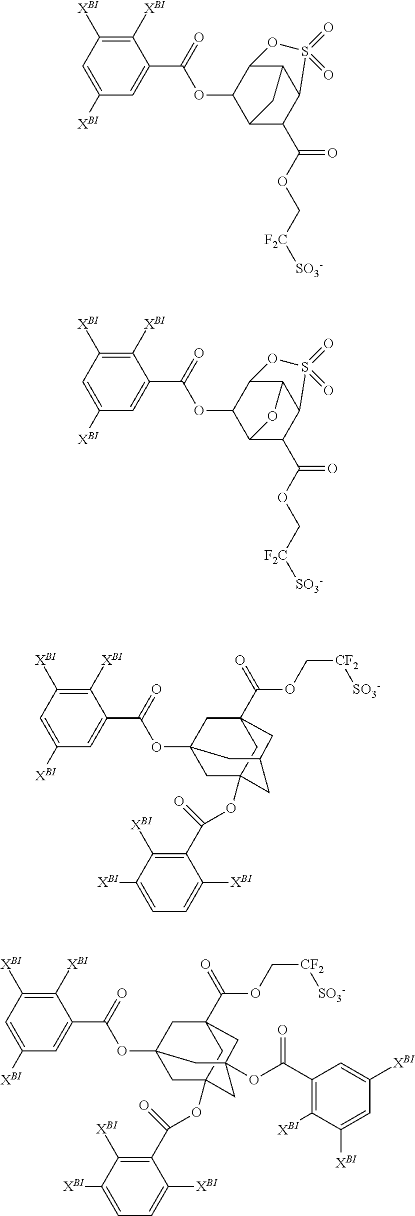

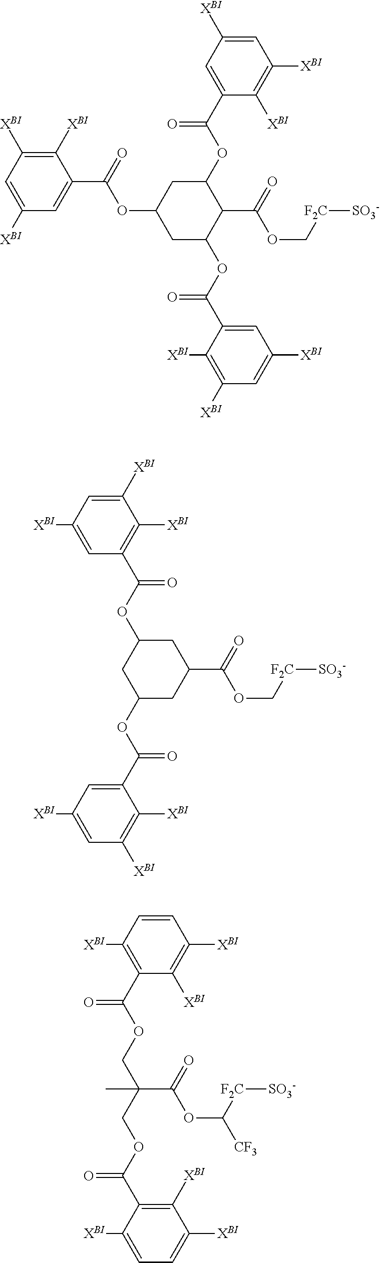

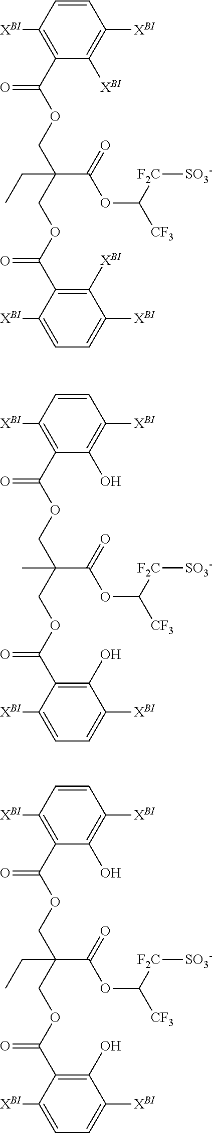

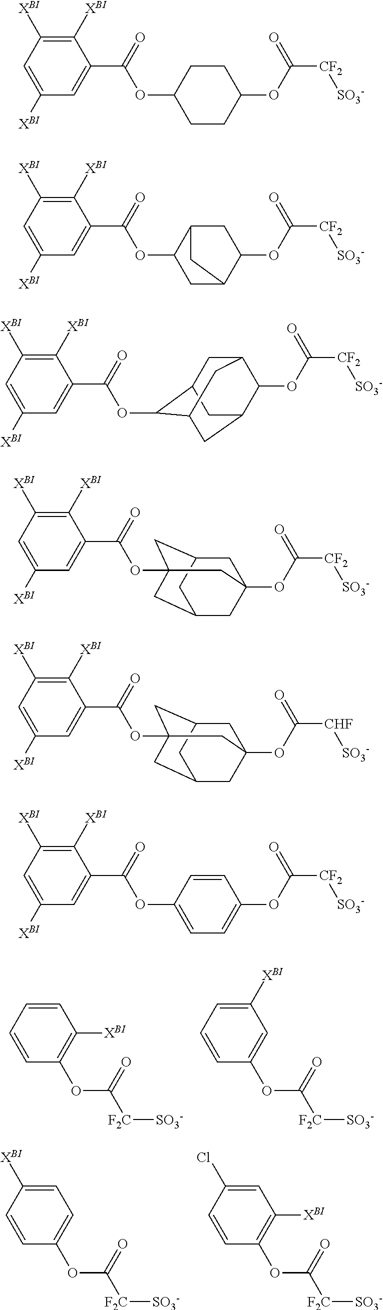

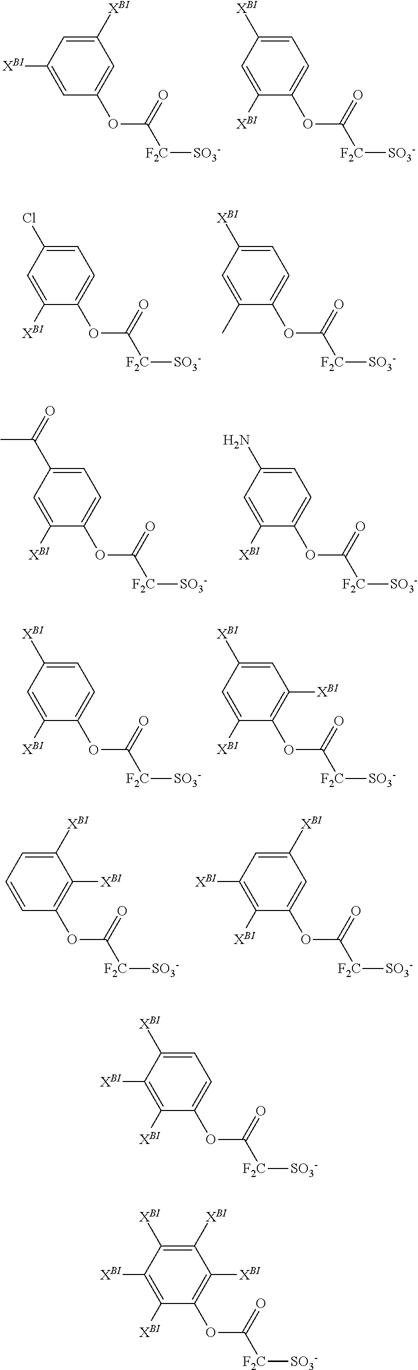

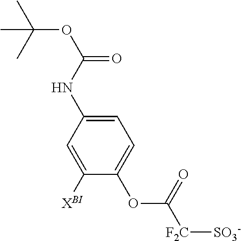

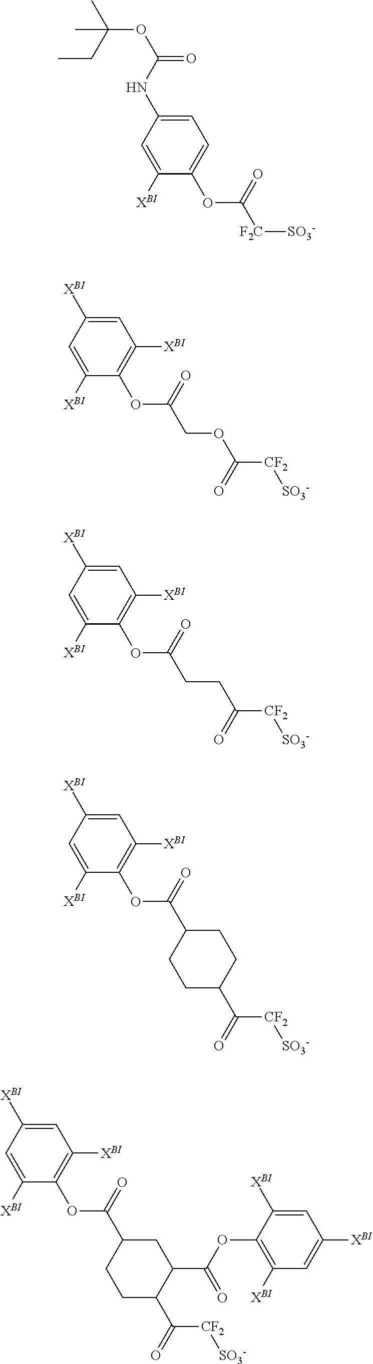

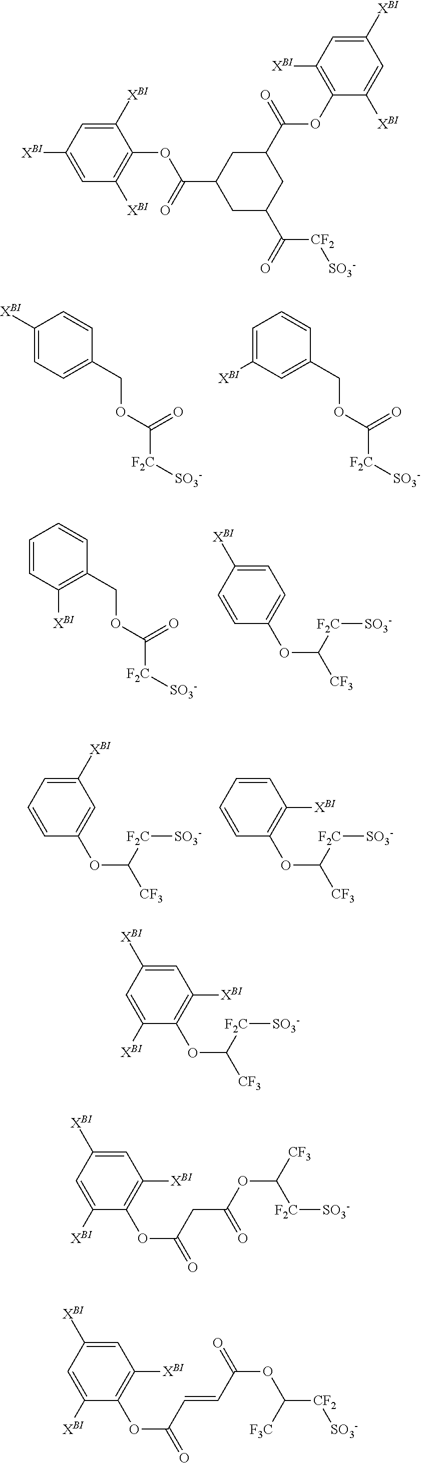

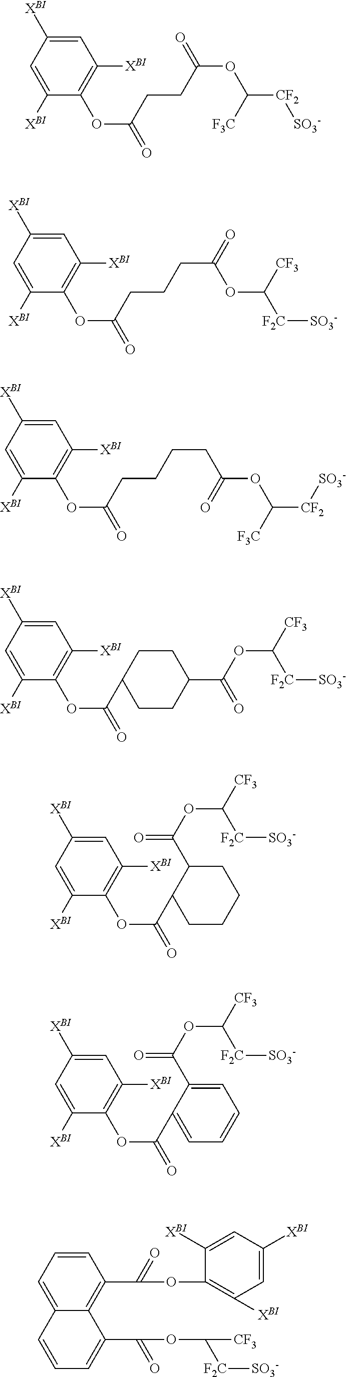

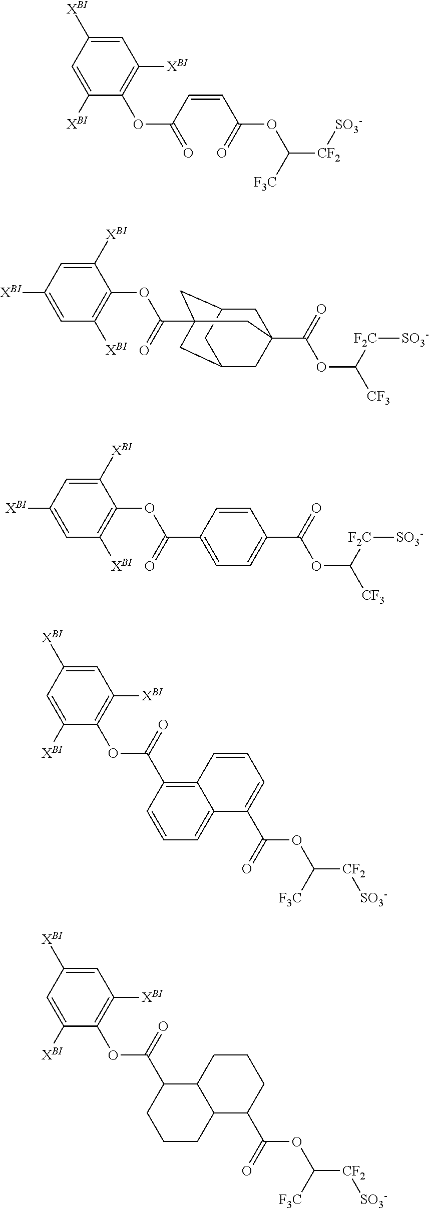

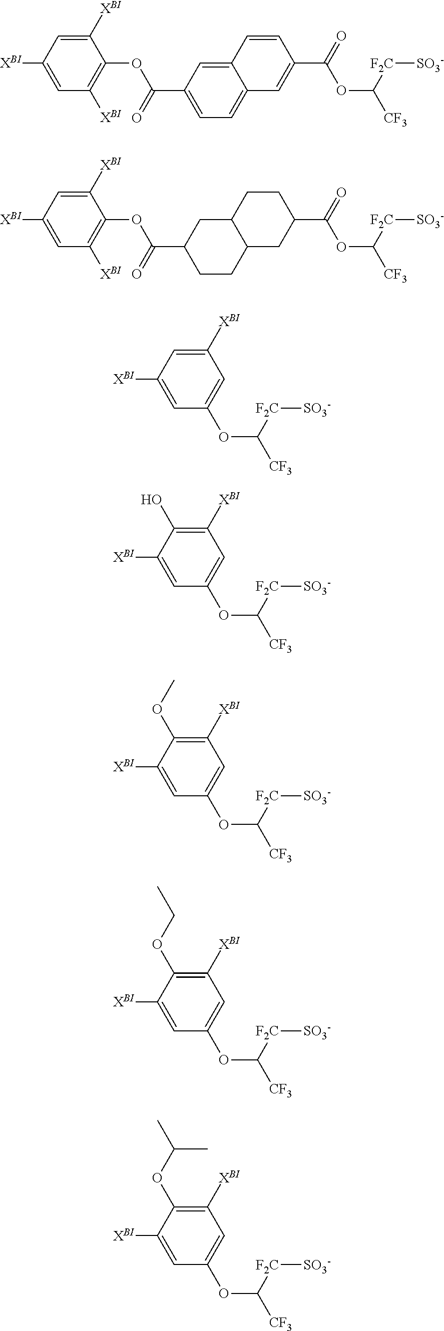

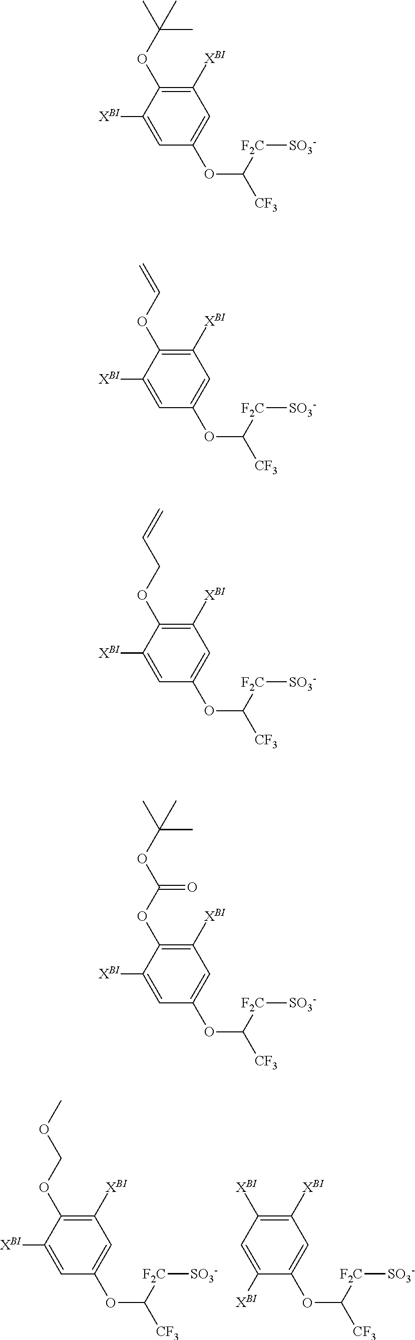

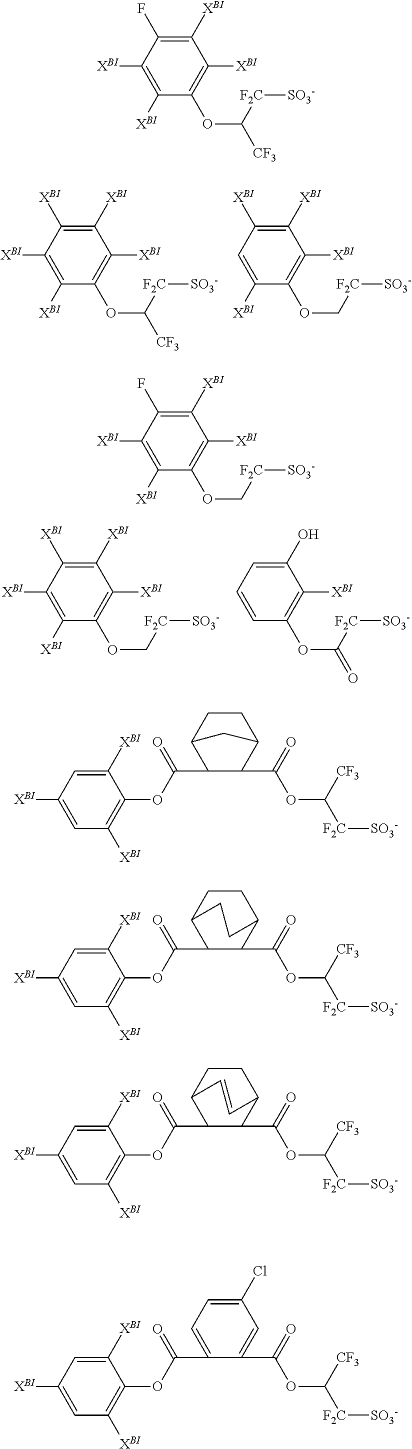

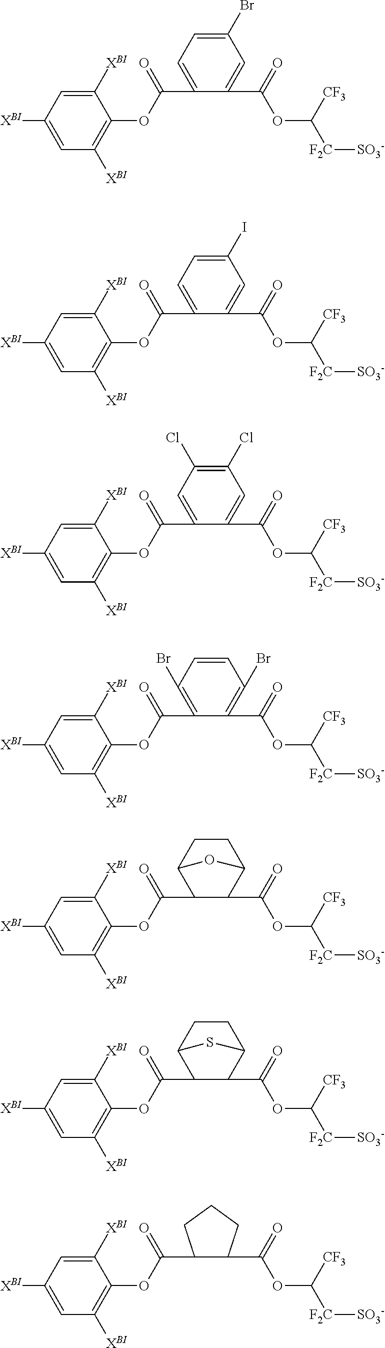

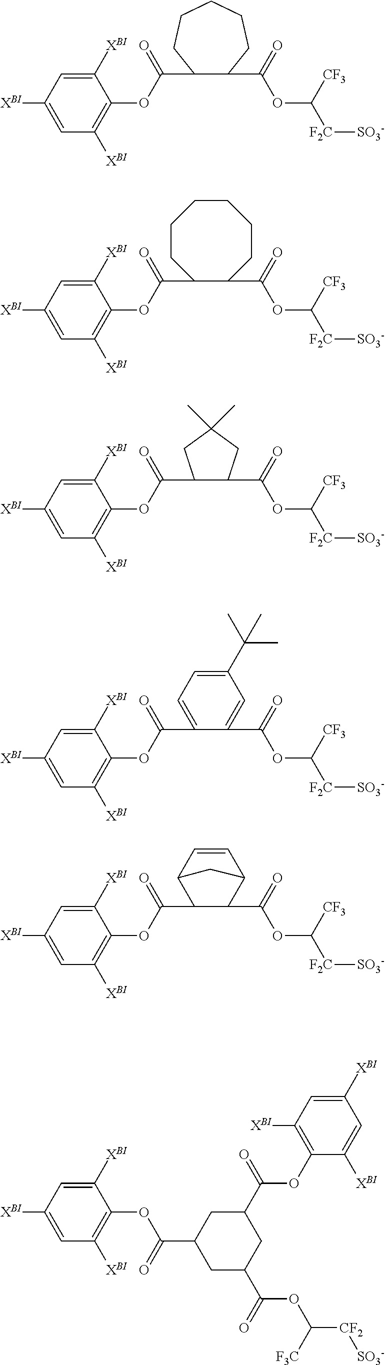

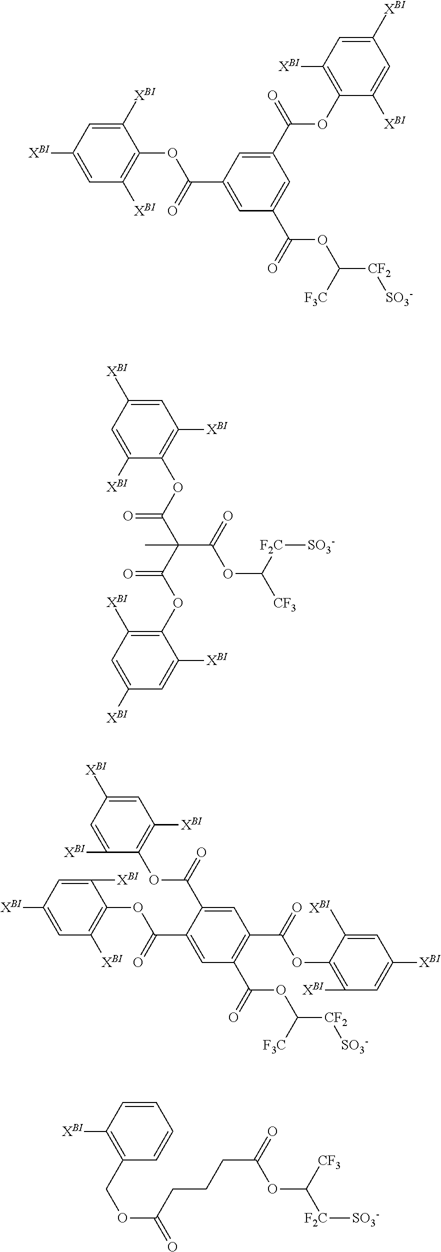

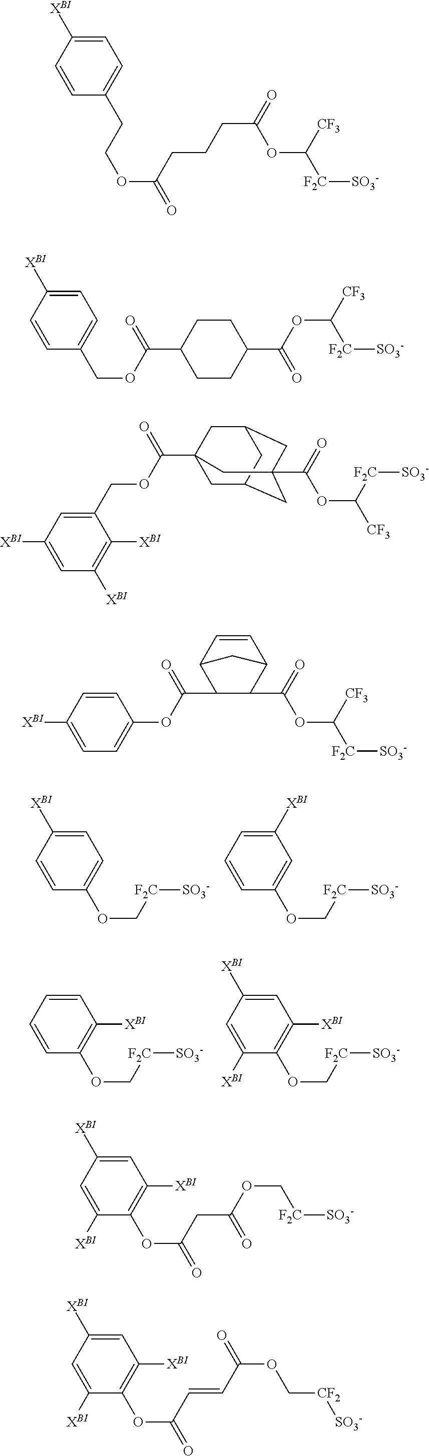

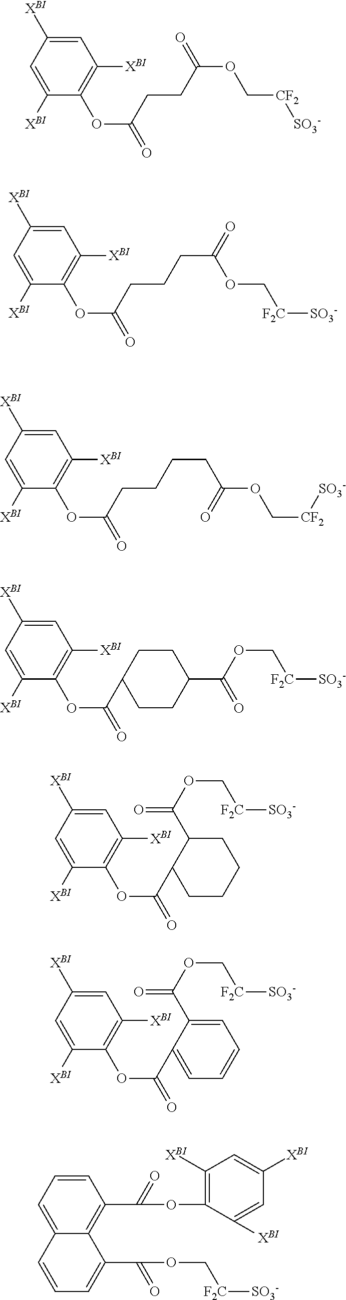

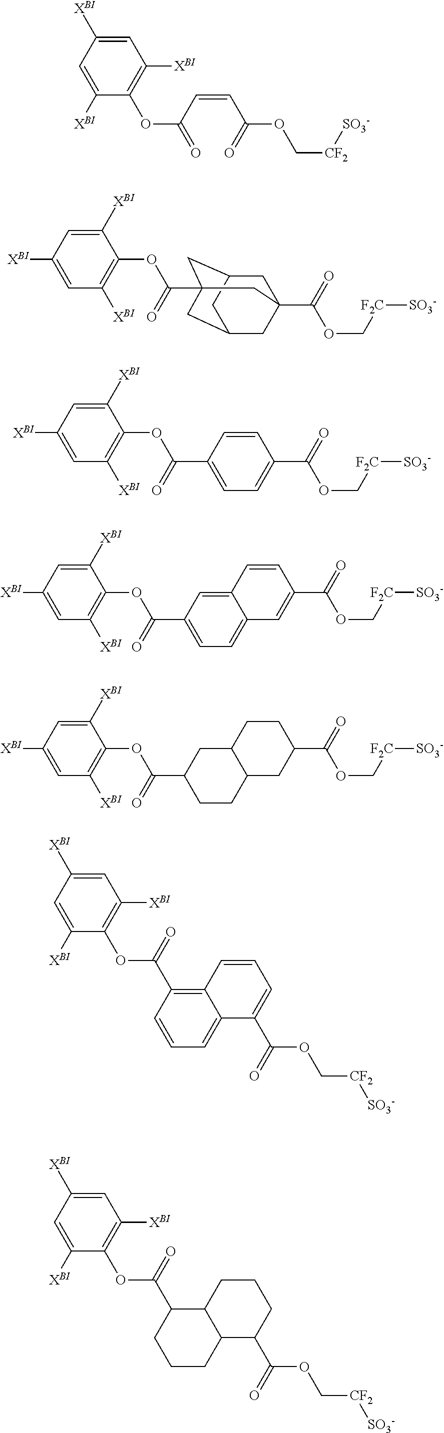

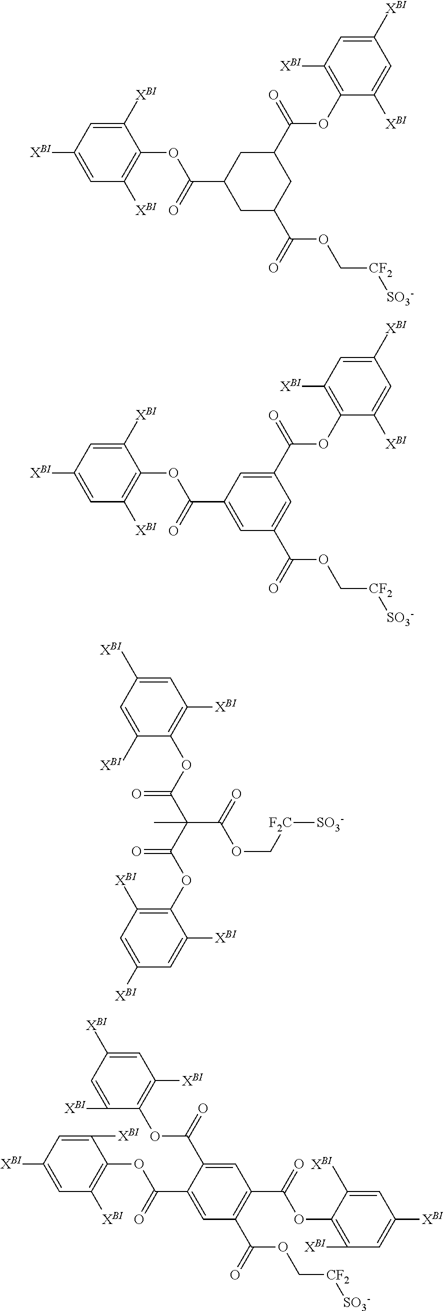

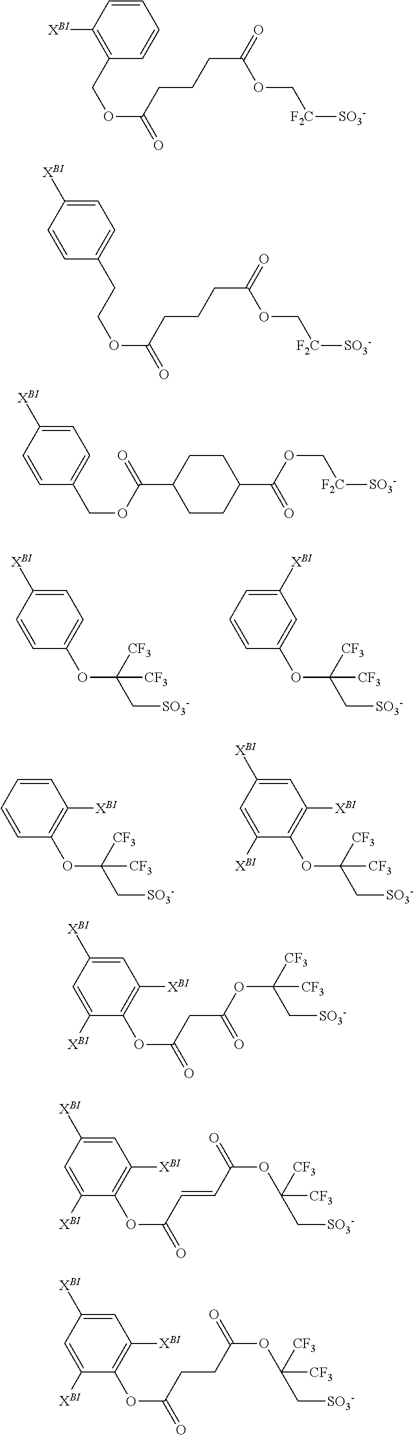

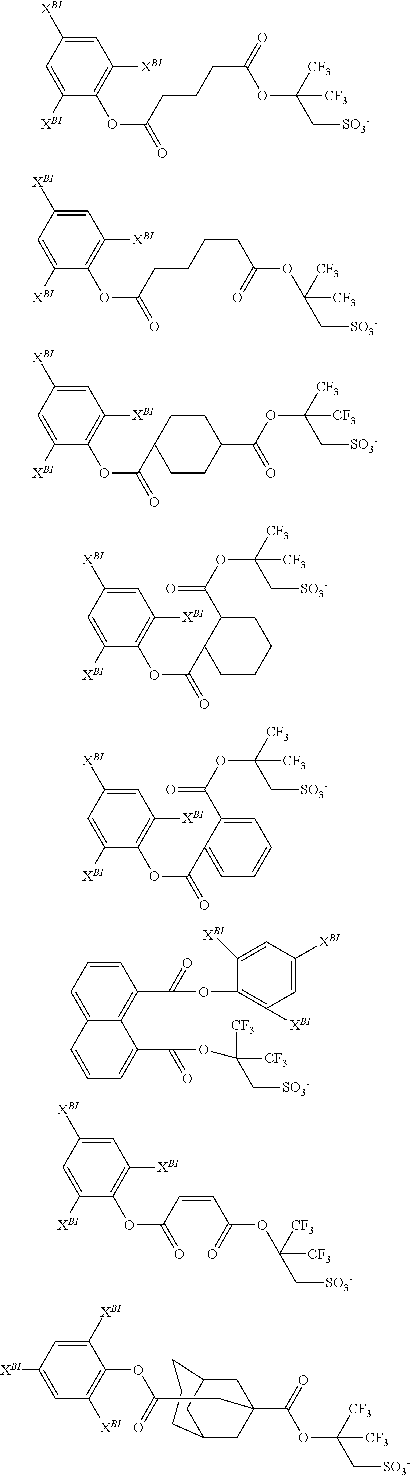

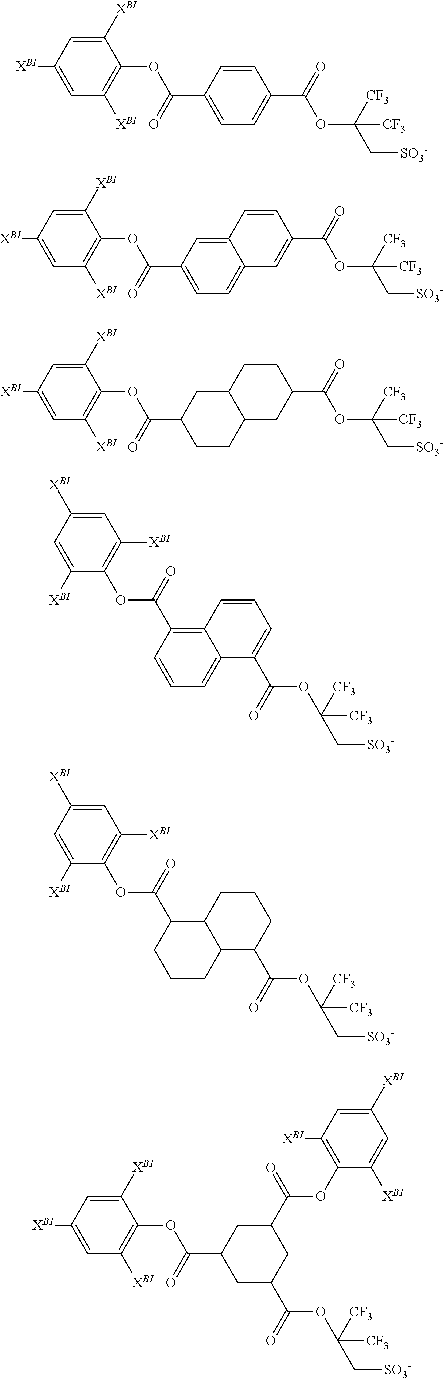

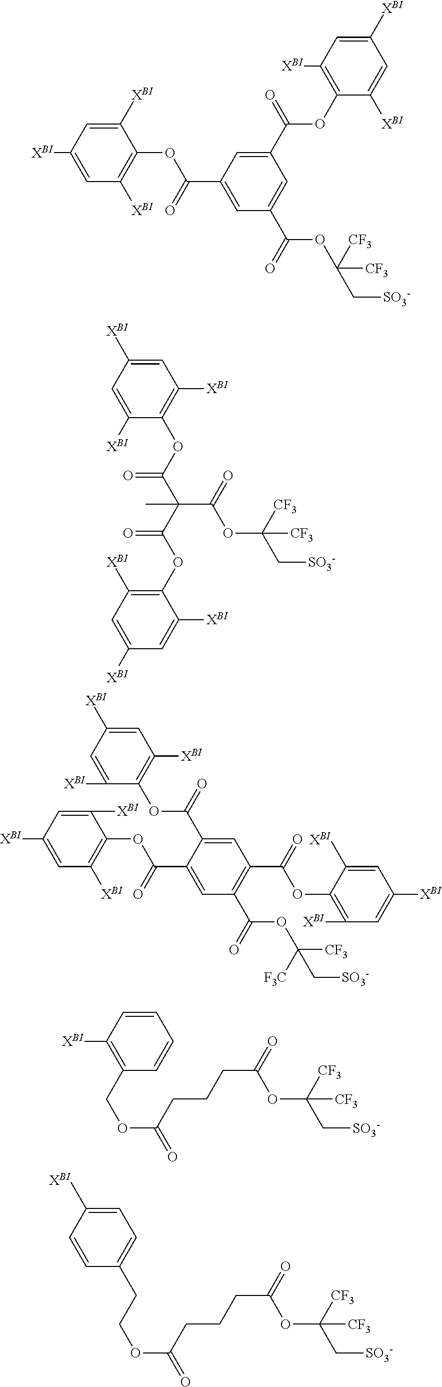

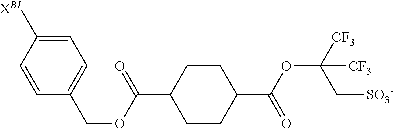

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-70692, anions having a cyclic ether group as described in JP-A 2018-180525 and JP-A 2021-35935, and anions as described in JP-A 2018-92159.

Further useful examples of the non-nucleophilic counter ion include an anion of a bulky fluorine-free benzenesulfonic acid derivative as described in JP-A 2006-276759, JP-A 2015-117200, JP-A 2016-65016, and JP-A 2019-202974, and fluorine-free benzenesulfonic acid or alkylsulfonic acid anions having an iodized aromatic group bonded thereto as described in JP 6645464.

Also useful examples of the non-nucleophilic counter ion include bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-24989.

The photoacid generator of the component (D) preferably has the following formula (5):

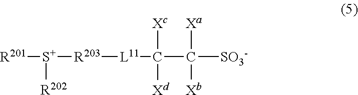

In formula (5), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with the sulfur atom to which they are bonded.

The C₁-C₃₀ hydrocarbyl group represented by R²⁰¹ to R²⁰² may be saturated or unsaturated and straight, branched, or cyclic. Specific examples thereof include C₁-C₃₀ alkyl groups such as a methyl group, an ethyl group, a n-propyl group, an isopropyl group, a n-butyl group, an isobutyl group, a sec-butyl group, a tert-butyl group, a tert-pentyl group, a n-pentyl group, a n-hexyl group, a n-octyl group, a 2-ethylhexyl group, a n-nonyl group, and a n-decyl group; C₃-C₃₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.02.6] decyl group, and an adamantyl group; C₆-C₃₀ aryl groups such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; and groups obtained by combining these. Some or all of the hydrogen atoms in the hydrocarbyl group may be substituted with a group containing a heteroatom such as an oxygen atom, a sulfur atom, a nitrogen atom, or a halogen atom, some of—CH₂-in the hydrocarbyl group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like.

The C₁-C₃₀ hydrocarbylene group represented by R²⁰³ may be saturated or unsaturated and straight, branched, or cyclic. Specific examples thereof include C₁-C₃₀ alkanediyl groups such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; C₃-C₃₀ cyclic saturated hydrocarbylene groups such as a cyclopentanediyl group, a cyclohexanediyl group, a norbornanediyl group, and an adamantanediyl group; and arylene groups such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group. Some or all of the hydrogen atoms in the hydrocarbylene group may be substituted with a group containing a heteroatom such as an oxygen atom, a sulfur atom, a nitrogen atom, or a halogen atom, some of-CH₂-in the hydrocarbylene group may be substituted with a group containing a heteroatom such as an oxygen atom, a sulfur atom, or a nitrogen atom, so that the group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, or the like. The heteroatom is preferably an oxygen atom.

In formula (5), L11 is a single bond, an ether bond, or a C₁-C₂₀ hydrocarbylene group which may contain a heteroatom. The hydrocarbylene group may be saturated or unsaturated, and may be straight, branched, or cyclic. Specific examples thereof are as exemplified above for the hydrocarbylene group represented by R²⁰³.

In formula (5), XA, XB, XC, and XD are each independently a hydrogen atom, a fluorine atom, or a trifluoromethyl group. Provided that at least one of Xa, Xb, Xe, and Xd is a fluorine atom or a trifluoromethyl group.

The photoacid generator having formula (5) preferably has the following formula (5′):

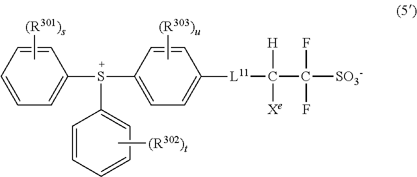

In formula (5′), L11 is as defined above. Xe is a hydrogen atom or a trifluoromethyl group, preferably a trifluoromethyl group. R³⁰¹, R³⁰², and R³⁰³ are each independently a hydrogen atom, or a C₁-C₂₀ hydrocarbyl group which may contain a heteroatom. The hydrocarbyl group may be saturated or unsaturated, and may be straight, branched, or cyclic. Specific examples thereof are as exemplified above for the hydrocarbyl group represented by R^{fa1} in formula (Xa-1-1). s and t are each independently 0, 1, 2, 3, 4, or 5, and u is 0, 1, 2, 3, or 4.

Examples of the photoacid generator having formula (5) are as exemplified for the photoacid generator having formula (2) in JP-A 2017-26980.

Among the photoacid generators, the photoacid generator containing the anion having formula (Xa-1-1) or (Xa-4) has small acid diffusion and excellent solubility in a solvent, which is particularly preferable. Those having formula (5′) are particularly preferred because of extremely reduced acid diffusion.

When chemically amplified resist composition of the present invention comprises the photoacid generator (D), the content thereof is preferably 0.1 to 40 parts by weight and more preferably 0.5 to 20 parts by weight per 80 parts by weight of the base polymer (A). When the added amount of the photoacid generator of the component (D) is in the above range, the resolution is favorable, and there is no possibility that a problem of foreign matter occurs after development or peeling of the resist film, which is preferable. The photoacid generator (D) may be used alone or in combination of two or more kinds thereof.

### Surfactant (E)

The chemically amplified resist composition of the present invention may further comprise a surfactant as a component (E). The surfactant (E) is preferably a surfactant insoluble or sparingly soluble in water and soluble in an alkaline developer, or a surfactant insoluble or sparingly soluble in water and an alkaline developer. As such a surfactant, those described in JP-A 2010-215608 and JP-A 2011-16746 can be referred to.

As the surfactant insoluble or sparingly soluble in water and an alkaline developer, among the surfactants described in the above publication, FC-4430 (manufactured by 3M), SURFLON® S-381 (manufactured by AGC Seimi Chemical Co., Ltd.), OLFINE® E1004 (manufactured by Nissin Chemical Industry Co., Ltd.), KH-20, KH-30 (manufactured by AGC Seimi Chemical Co., Ltd.), an oxetane ring-opening polymer having the following formula (surf-1), and the like are preferable.

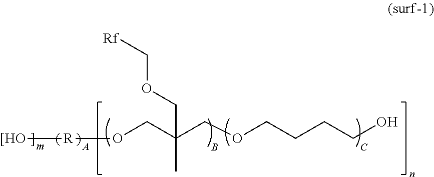

R, R^{f}, A, B, C, m, and n apply only to formula (surf-1), regardless of the foregoing description. R is a di to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group, and examples of the tri- and tetra-valent aliphatic group are shown below.

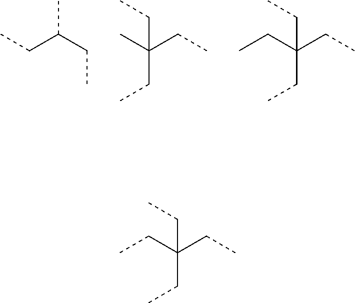

wherein the broken line denotes a point of attachment, and these formulae are partial structures derived from glycerol, trimethylolethane, trimethylolpropane, and pentaerythritol.

Of these, a 1,4-butylene group, a 2,2-dimethyl-1,3-propylene group, and the like are preferable.

Rf is a trifluoromethyl group or a pentafluoroethyl group, preferably a trifluoromethyl group. m is an integer of 0 to 3, n is an integer of 1 to 4, and the sum of n and m, which represents the valence of R, is an integer of 2 to 4. A is 1. B is an integer of 2 to 25, preferably an integer of 4 to 20. C is an integer of 0 to 10, preferably 0 or 1. Each constituent unit in formula (surf-1) does not prescribe the arrangement thereof, and may be arranged either blockwise or randomly. For the preparation of surfactants in the form of partially fluorinated oxetane ring-opened polymers, reference should be made to U.S. Pat. No. 5,650,483 and the like.

When a resist protective film is not used in ArF immersion lithography, the surfactant insoluble or sparingly soluble in water and soluble in an alkaline developer has a function of minimizing water penetration or leaching by being oriented on the surface of the resist film. Therefore, the surfactant is useful for preventing water-soluble components from being leached out of the resist film for minimizing any damage to the exposure tool, and is also useful because it becomes solubilized during alkaline aqueous solution development after exposure or after post exposure bake (PEB), and thus forms few or no foreign matter which become defects. Such a surfactant has a property of being insoluble or sparingly soluble in water and being soluble in an alkaline developer, is also called a polymeric surfactant, and is particularly preferably a surfactant having high water repellency and improving lubricity.

Specific examples of such a polymeric surfactant include those containing at least one selected from repeat units having any one of the following formulae (6A) to (6E).

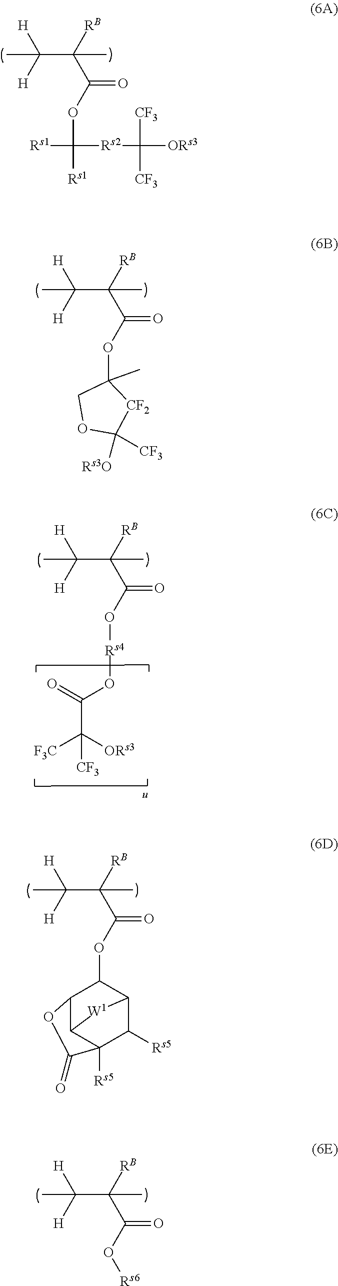

In formulae (6A) to (6E), RB is a hydrogen atom, a fluorine atom, a methyl group, or a trifluoromethyl group. W1 is —CH₂—, —CH₂CH₂—, —O—, or two separate —H. R^{s1} is each independently a hydrogen atom or a C₁-C₁₀ hydrocarbyl group. R^{s2} is a single bond or a C₁-C₅ straight or branched hydrocarbylene group. R^{s3} is each independently a hydrogen atom, a C₁-C₁₅ hydrocarbyl group or fluorinated hydrocarbyl group, or an acid labile group. When R^{s3} is a hydrocarbyl group or fluorinated hydrocarbyl group, an ether bond or a carbonyl group may intervene in a carbon-carbon bond. R^{s4} is a C₁-C₂₀ (w+1)-valent hydrocarbon group or fluorinated hydrocarbon group. w is 1, 2, or 3. R^{s5} is each independently a hydrogen atom or a group having-C(═O)—O—Rsa. Rsa is a C₁-C₂₀ fluorinated hydrocarbyl group. R^{s6} is a C₁-Cis hydrocarbyl group or fluorinated hydrocarbyl group, and an ether bond or a carbonyl group may intervene in a carbon-carbon bond thereof.

The C₁-C₁₀ hydrocarbyl group represented by R^{s1} is preferably a saturated hydrocarbyl group and may be straight, branched, or cyclic. Specific examples thereof include C₁-C₁₀ alkyl groups such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, and a n-decyl group; and C₃-C₁₀ cyclic saturated hydrocarbyl groups such as a cyclopropyl group, a cyclobutyl group, a cyclopentyl group, a cyclohexyl group, an adamantyl group, and a norbornyl group. Of these, C₁-C₆ groups are preferable.

The hydrocarbylene group represented by R^{s2} is preferably a saturated hydrocarbylene group and may be straight, branched, or cyclic. Specific examples thereof include a methylene group, an ethylene group, a propylene group, a butylene group, and a pentylene group.

The hydrocarbyl group represented by R^{s3} or R^{s6} may be saturated or unsaturated and straight, branched, or cyclic. Specific examples thereof include aliphatic unsaturated hydrocarbyl groups such as a saturated hydrocarbyl group, an alkenyl group, and an alkynyl group, and a saturated hydrocarbyl group is preferable. Examples of the saturated hydrocarbyl group include an undecyl group, a dodecyl group, a tridecyl group, a tetradecyl group, and a pentadecyl group in addition to those exemplified as the hydrocarbyl group represented by R^{s1}. Examples of the fluorinated hydrocarbyl group represented by R^{s3} or R^{s6} include groups in which some or all hydrogen atoms bonded to carbon atoms of the foregoing hydrocarbyl group are substituted by fluorine atoms. As described above, an ether bond or a carbonyl group may be interposed between these carbon-carbon bonds.

Specific examples of the acid labile group represented by R^{s3} include the groups having formulae (AL-3) to (AL-5) described above, trialkylsilyl groups in which each alkyl group is a C₁-C₆ alkyl group, and C₄-C₂₀ oxo group-containing alkyl groups.

The (w+1)-valent hydrocarbon or fluorinated hydrocarbon group represented by R^{s4} may be straight, branched, or cyclic, and specific examples thereof include the foregoing hydrocarbyl or fluorinated hydrocarbyl groups from which “w” number of hydrogen atoms are eliminated.

The fluorinated hydrocarbyl group represented by Rsa is preferably saturated and may be straight, branched, or cyclic. Specific examples thereof include the foregoing hydrocarbyl groups in which some or all hydrogen atoms are substituted by fluorine atoms, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeat units having any one of formulae (6A) to (6E) are shown below, but not limited thereto. In the following formula. RB is as defined above.

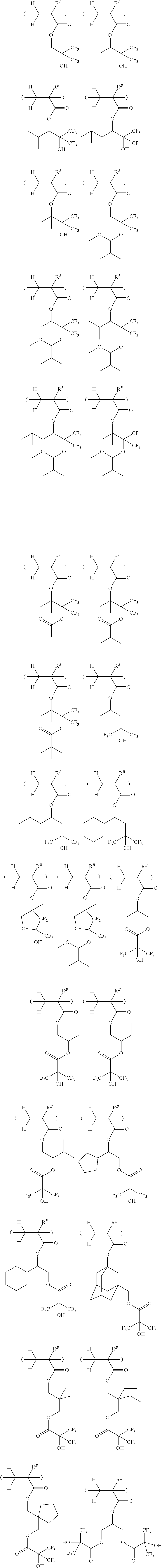

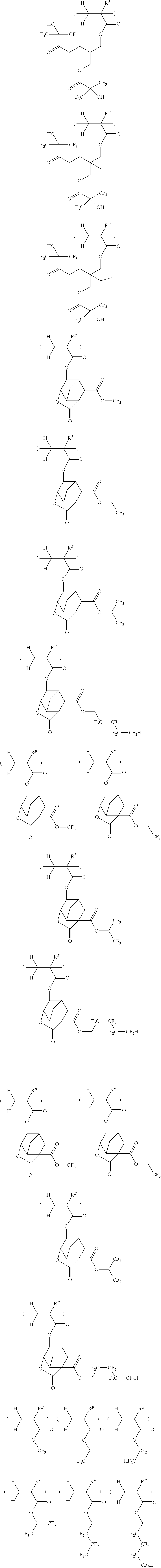

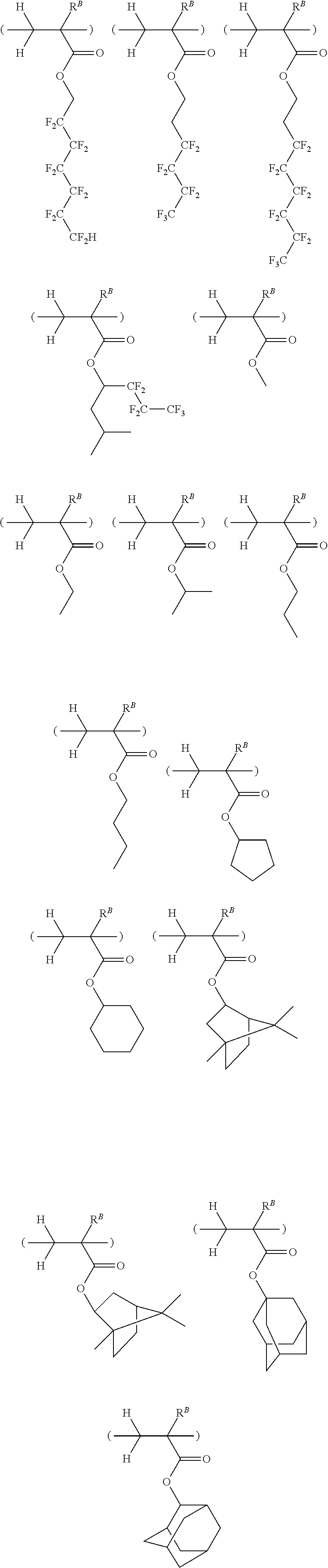

The polymeric surfactant may further contain repeat units other than the repeat units having formulae (6A) to (6E). Examples of the other repeat units include repeat units derived from methacrylic acid, an a-trifluoromethylacrylic acid derivative, or the like. In the polymeric surfactant, the content of the repeat units having formulae (6A) to (6E) is preferably 20 mol % or more, more preferably 60 mol % or more, and still more preferably 100 mol % of the overall repeat units.

The Mw of the polymeric surfactant is preferably 1000 to 500000, more preferably 3000 to 100000. Mw/Mn is preferably 1.0 to 2.0, more preferably 1.0 to 1.6.

Examples of the method for synthesizing the polymeric surfactant include a method of dissolving an unsaturated bond-containing monomer providing repeat units having formulae (6A) to (6E) and optionally other repeat units in an organic solvent, adding a radical initiator, and heating for polymerization. Examples of the organic solvent used in the polymerization include toluene, benzene, THF, diethyl ether, and dioxane. Examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or polymerization may be followed by protection or partial protection.

In the case of synthesizing the polymeric surfactant, any known chain transfer agent such as dodecyl mercaptan or 2-mercaptoethanol may be used for molecular weight control purpose. In this case, these chain transfer agents are preferably added in an amount of 0.01 to 10 mol % based on the total number of moles of monomers to be polymerized.

When the chemically amplified resist composition of the present invention comprises the surfactant (E), the content thereof is preferably 0.1 to 50 parts by weight and more preferably 0.5 to 10 parts by weight per 80 parts by weight of the base polymer (A). When the content of the surfactant (E) is 0.1 parts by weight or more, the receding contact angle with water of the resist film at its surface is sufficiently improved, and when the content thereof is 50 parts by weight or less, the dissolution rate of the resist film at its surface in the developer is low, and the height of the formed fine pattern is sufficiently maintained. The surfactant (E) may be used alone or in combination of two or more kinds thereof.

### Other Components (F)

The chemically amplified resist composition of the present invention may comprise a compound which is decomposed with an acid to generate another acid (acid amplifier compound), an organic acid derivative, a fluorinated alcohol, a compound having a Mw of 3000 or less which changes its solubility in a developer under the action of acid (dissolution inhibitor), and the like as other components (F). As the acid amplifier compound, a compound described in JP-A 2009-269953 or JP-A 2010-215608 can be referred to. When the chemically amplified resist composition comprises the acid amplifier compound, the content thereof is preferably 0 to 5 parts by weight and more preferably 0 to 3 parts by weight per 80 parts by weight of the base polymer (A). When the content thereof is too large, it is difficult to control acid diffusion, and resolution and pattern profile may be deteriorated. As the organic acid derivative, the fluorinated alcohol, and the dissolution inhibitor, compounds described in JP-A 2009-269953 or JP-A 2010-215608 can be referred to.

### Pattern Forming Process

A pattern forming process of the present invention comprises the steps of: applying the chemically amplified resist composition defined above onto a substrate to form a resist film thereon; exposing the resist film to high-energy radiation; and developing the exposed resist film in a developer.

As the substrate, for example, substrates for integrated circuit fabrication (such as Si, SiO₂, SiN, SiON, TiN, WSi, BPSG, SOG, and organic antireflective coating), or substrates for mask circuit fabrication (such as Cr, CrO, CrON, MoSi₂, and SiO₂) can be used.

The resist film can be formed by, for example, applying the chemically amplified resist composition onto a substrate by a method such as spin coating so that the film thickness is preferably 0.05 to 2 μm, and prebaking the chemically amplified resist composition on a hotplate at preferably 60 to 150° C. for 1 to 10 minutes, more preferably 80 to 140° C. for 1 to 5 minutes.

Examples of the high-energy radiation used for exposure of the resist film include KrF excimer laser, ArF excimer laser, EB, and EUV having a wavelength of 3 to 15 nm. In the case of using KrF excimer laser, ArF excimer laser, or EUV, exposure can be performed by using a mask for forming a target pattern and performing irradiation so that the exposure dose is preferably 1 to 200 mJ/cm², more preferably 10 to 100 mJ/cm². In the case of using EB, irradiation is performed using a mask for forming a target pattern or directly so that the exposure dose is preferably 1 to 300 uC/cm², more preferably 10 to 200 μC/cm².

In addition to a normal exposure method, it is also possible to use an immersion method in which exposure is performed by interposing a liquid having a refractive index of 1.0 or more between a resist film and a projection lens. In this case, it is also possible to use a water-insoluble protective film.

The water-insoluble protective film is used to prevent an eluate from the resist film and to increase the lubricity of the film surface, and is generally divided into two types. The first type is an organic solvent-strippable protective film in which peeling is required before alkaline aqueous solution development by an organic solvent that does not dissolve a resist film, and the second type is an alkaline aqueous solution-soluble protective film which is soluble in an alkaline developer so that the protective film is removed simultaneously with the removal of solubilized regions of the resist film. The protective film of the second type is particularly preferably of a material comprising a polymer having a 1,1,1,3,3,3-hexafluoro-2-propanol residue, which is insoluble in water and soluble in an alkaline developer, as a base and dissolved in an alcohol-based solvent of 4 or more carbon atoms, an ether-based solvent of 8 to 12 carbon atoms, and a mixed solvent thereof. A material obtained by dissolving the surfactant, which is insoluble in water and soluble in an alkaline developer, in an alcohol-based solvent of 4 or more carbon atoms, an ether-based solvent of 8 to 12 carbon atoms, or a mixed solvent thereof can also be used.

After the exposure, PEB may be performed. PEB can be performed, for example, by heating on a hotplate at preferably 60 to 150° C. for 1 to 5 minutes, more preferably 80 to 140° C. for 1 to 3 minutes.

For example, the resist film is developed with a developer in the form of an alkaline aqueous solution such as tetramethylammonium hydroxide (TMAH) in an amount of preferably 0.1 to 5 wt %, more preferably 2 to 3 wt % for preferably 0.1 to 3 minutes, more preferably 0.5 to 2 minutes by conventional techniques such as dip, puddle, and spray techniques. In this way, the exposed area is dissolved and a target pattern is formed on the substrate.

After the resist film is formed, the acid generator or the like may be extracted from the film surface by performing rinsing with pure water, or particles may be washed off, or rinsing for removing water remaining on the film after exposure may be performed.

Pattern formation may be performed by a double patterning process. Examples of the double patterning process include a trench process of processing an underlay to 1:3 trench pattern by a first step of exposure and etching, shifting the position, and forming a 1:3 trench pattern by a second step of exposure, for forming a 1:1 pattern, and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process of the present invention, a method of negative tone development in which an organic solvent is used instead of the alkaline aqueous solution as the developer for dissolving away the unexposed area may be used.

For the organic solvent development, as the developer, 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, 2-phenylethyl acetate, and the like can be used. These organic solvents may be used alone or in admixture of two or more kinds thereof.

### EXAMPLES

Hereinafter, the present invention is specifically described with reference to Synthesis Examples, Examples, and Comparative Examples, but the present invention is not limited to the following Examples. The devices used are as follows.

MALDI TOF-MS: S3000 manufactured by JEOL Ltd.

Synthesis of sulfonium salt type monomer

### Example 1-1

Synthesis of Monomer PAG-1

### (1) Synthesis of Intermediate In-1

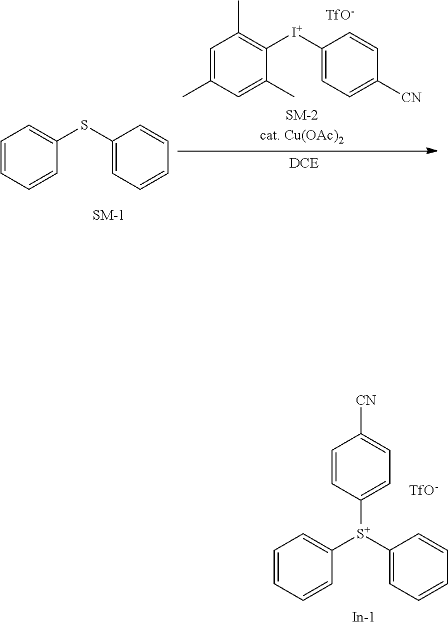

In a nitrogen atmosphere, SM-1 (27.9 g), SM-2 (89.5 g), and copper acetate (8.24 g) were dissolved in dichloroethane (300 g). Thereafter, the reaction system was heated to 100° C. and aged for 15 hours. After aging, the reaction system was cooled and water (150 g) was added to stop the reaction. Thereafter, the organic layer was separated, washed with water, and then concentrated under reduced pressure to distill off the solvent. The residue was purified by silica gel column chromatography to obtain 53.8 g of Intermediate In-1 as a yellow viscous oily product (yield: 82%).

### (2) Synthesis of Monomer PAG-1

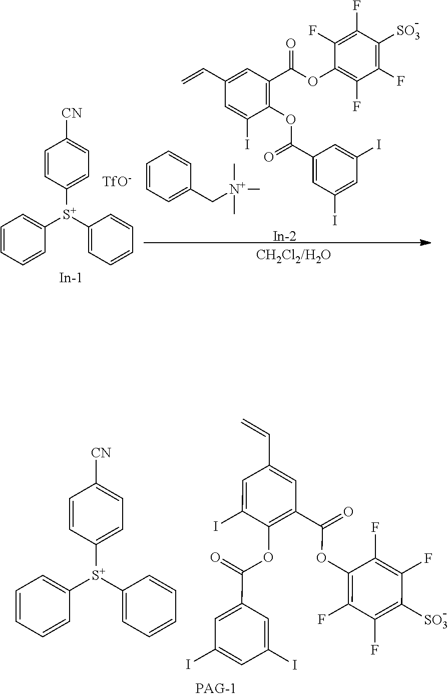

In a nitrogen atmosphere, Intermediate In-1 (36.1 g), Intermediate In-2 (76.7 g), methylene chloride (300 g), and water (150 g) were charged, and the mixture was stirred at room temperature for 30 minutes. The organic layer was separated, washed with water, and then concentrated under reduced pressure. The residue was washed with diisopropyl ether and concentrated to obtain 83.6 g of Monomer PAG-1 as an oily product (yield: 96%).

The TOF-MS results for PAG-1 are shown below.

MALDI TOF-MS:
POSITIVE M+288 (corresponding to C₁₉H₁₄NS⁺)
NEGATIVE M−873 (corresponding to C₂₂H₈F₄I₇O₇S⁻)

### Examples 1-2 to 1-9

### Synthesis of Monomers PAG-2 to PAG-9

The following sulfonium salt type monomers PAG-2 to PAG-9 were synthesized by corresponding raw materials and various organic synthesis reactions.

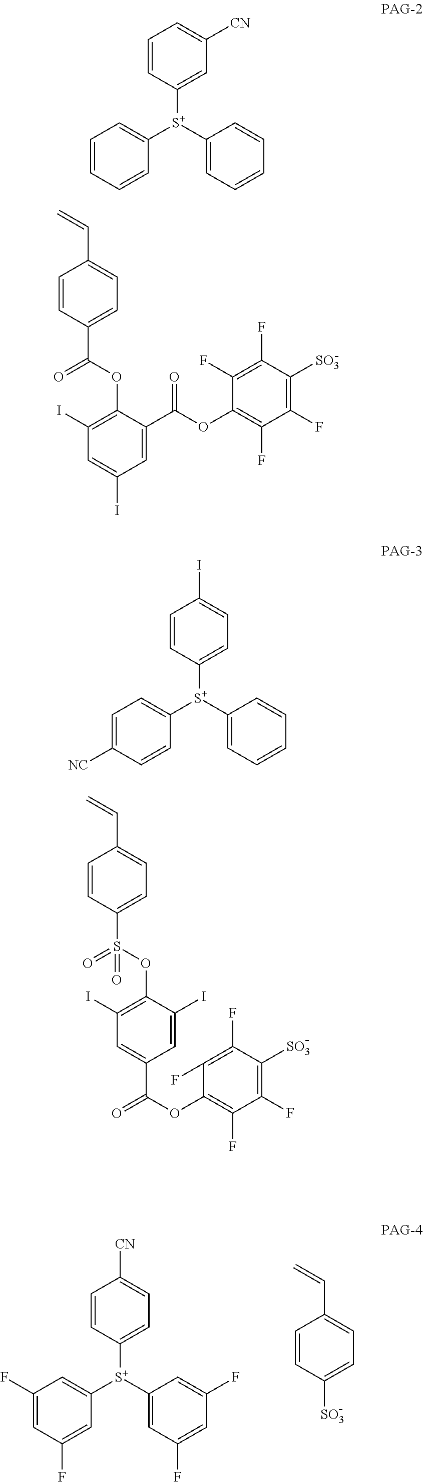

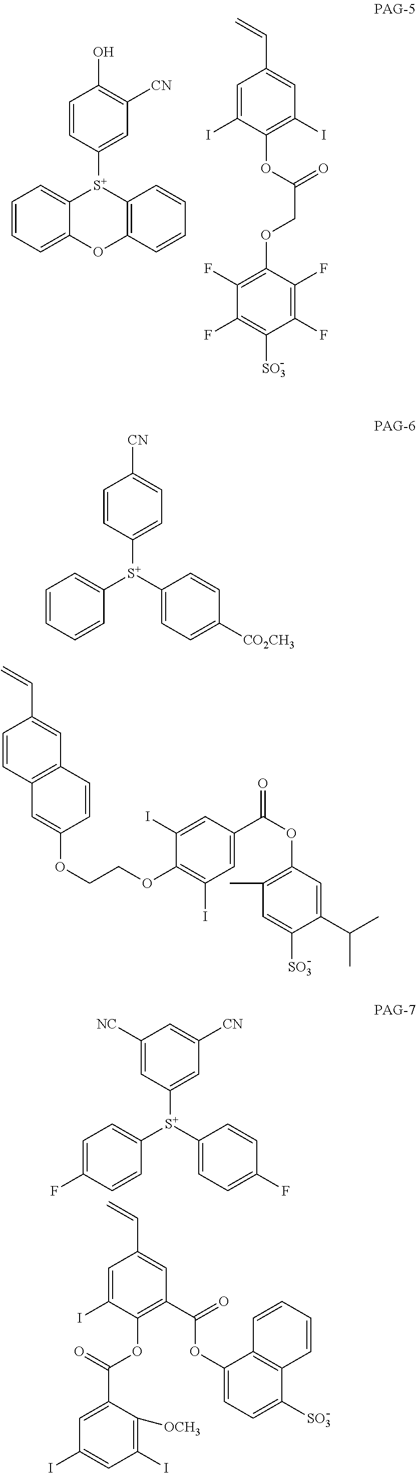

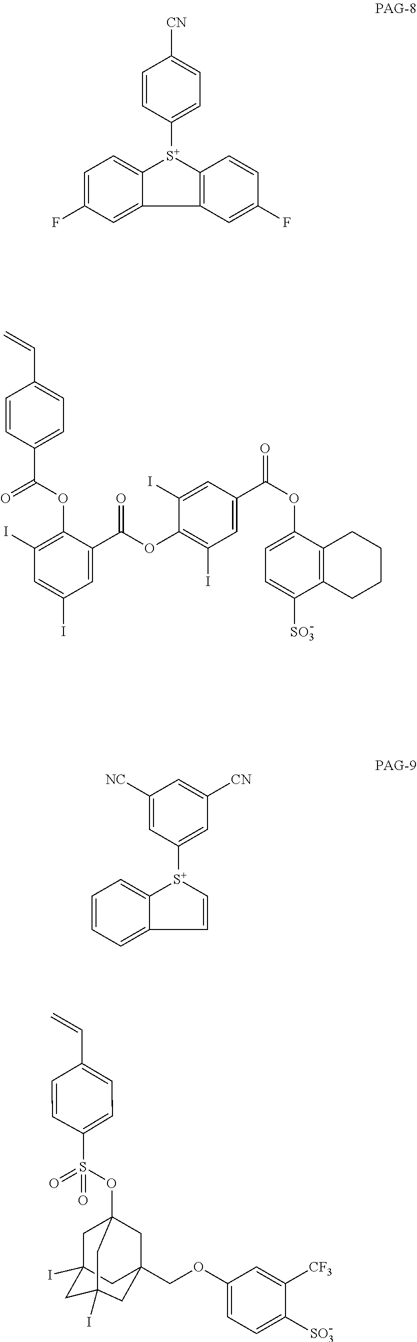

### Comparative Examples 1-1 to 1-6

### Synthesis of Comparative Monomers PAG-A to PAG-F

The following comparative sulfonium salt type monomers PAG-A to PAG-F were synthesized by corresponding raw materials and various organic synthesis reactions.

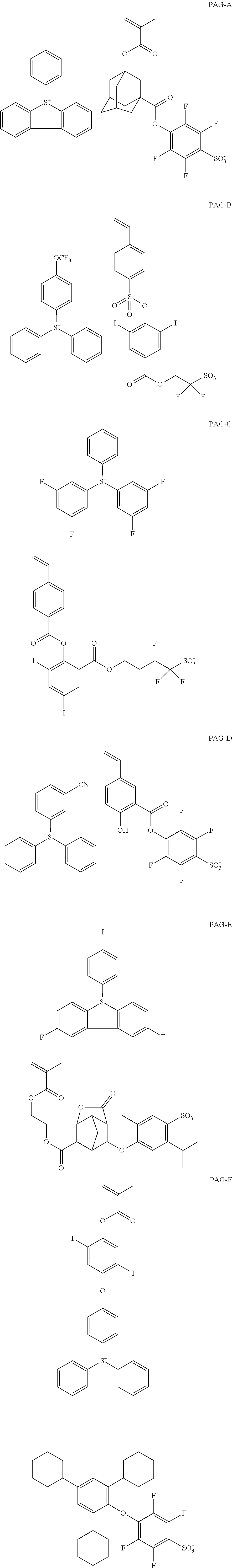

### Synthesis of Base Polymer

Among the monomers used for synthesis of the base polymer, monomers other than PAG-1 to PAG-9 and PAG-A to PAG-F are as follows.

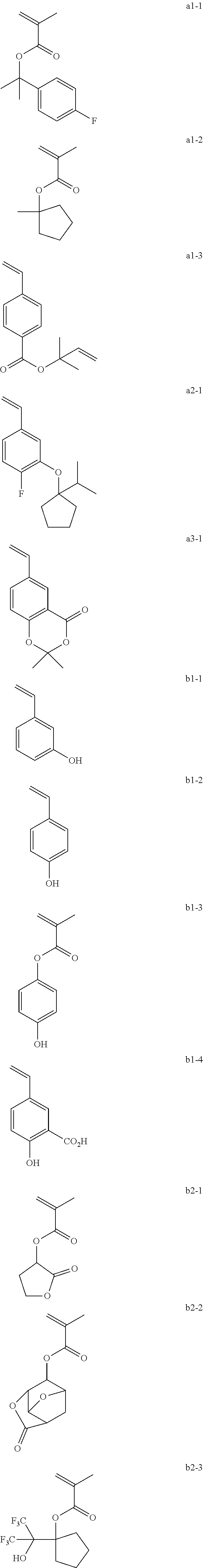

### Example 2-1

### Synthesis of Polymer P-1

In a nitrogen atmosphere, a flask was charged with Monomer a1-1 (18.5 g), Monomer b1-1 (5.4 g), Monomer PAG-1 (26.2 g), 1.73 g of V-601 (manufactured by Wako Pure Chemical Industries, Ltd.), and 70 g of MEK to prepare a monomer-polymerization initiator solution. Another flask under a nitrogen atmosphere was charged with 23 g of MEK, which was heated to 80° C. with stirring, and then the monomer-polymerization initiator solution was added dropwise over 4 hours. After completion of the dropwise addition, stirring was continued for 2 hours while maintaining the temperature of the polymerization liquid at 80° C., and then the polymerization liquid was cooled to room temperature. The obtained polymerization liquid was added dropwise to 1000 g of hexane with vigorous stirring, and the precipitated polymer was separated by filtration. The obtained polymer was washed twice with 300 g of hexane, and then vacuum-dried at 50° C. for 20 hours to obtain Polymer P-1 in a white powder form (amount: 49.1 g, yield: 98%). The Mw of Polymer P-1 was 9600, and Mw/Mn was 1.62. The Mw is a value measured in terms of polystyrene by GPC using DMF as a solvent.

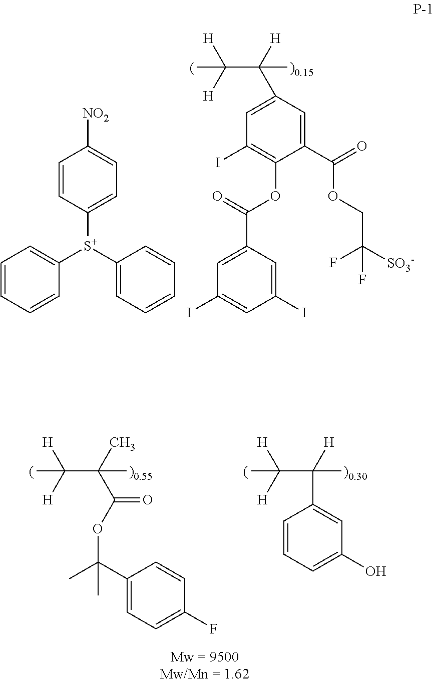

### Examples 2-2 to 2-26 and Comparative Examples 2-1 to 2-18

### Synthesis of Polymers P-2 to P-26 and CP-1 to CP-18

Polymers shown in Tables 1 and 2 were produced in the same manner as in Example 2-1, except that the type and blending ratio of each monomer were changed.

TABLE 1

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | PAG-1 | 15 | a1-1 | 55 | b1-1 | 30 | — | — | — | — | 9,600 | 1.62 |
| P-2 | PAG-2 | 15 | a1-1 | 55 | b1-1 | 30 | — | — | — | — | 9,300 | 1.62 |
| P-3 | PAG-3 | 15 | a1-1 | 55 | b1-1 | 30 | — | — | — | — | 9,400 | 1.63 |
| P-4 | PAG-4 | 15 | a1-1 | 55 | b1-1 | 30 | — | — | — | — | 9,400 | 1.63 |
| P-5 | PAG-5 | 15 | a1-1 | 55 | b1-1 | 30 | — | — | — | — | 9,200 | 1.61 |
| P-6 | PAG-6 | 15 | a1-1 | 55 | b1-1 | 30 | — | — | — | — | 9,300 | 1.62 |
| P-7 | PAG-7 | 15 | a1-1 | 55 | b1-1 | 30 | — | — | — | — | 9,200 | 1.65 |
| P-8 | PAG-8 | 15 | a1-1 | 55 | b1-1 | 30 | — | — | — | — | 9,400 | 1.61 |
| P-9 | PAG-9 | 15 | a1-1 | 55 | b1-1 | 30 | — | — | — | — | 9,300 | 1.62 |
| P-10 | PAG-1 | 15 | a1-2 | 55 | b1-1 | 30 | — | — | — | — | 9,400 | 1.61 |
| P-11 | PAG-1 | 15 | a1-3 | 55 | b1-1 | 30 | — | — | — | — | 9,200 | 1.63 |
| P-12 | PAG-1 | 15 | a2-1 | 55 | b1-1 | 30 | — | — | — | — | 9,800 | 1.62 |
| P-13 | PAG-1 | 15 | a3-1 | 45 | b1-1 | 40 | — | — | — | — | 9,000 | 1.63 |
| P-14 | PAG-2 | 15 | a1-1 | 55 | b1-2 | 30 | — | — | — | — | 9,100 | 1.61 |
| P-15 | PAG-2 | 15 | a1-1 | 55 | b1-3 | 30 | — | — | — | — | 9,200 | 1.62 |
| P-16 | PAG-2 | 15 | a1-1 | 55 | b1-4 | 30 | — | — | — | — | 9,100 | 1.64 |
| P-17 | PAG-1 | 15 | a1-1 | 30 | a2-1 | 20 | b1-1 | 35 | — | — | 9,200 | 1.61 |
| P-18 | PAG-3 | 15 | a1-1 | 35 | a3-1 | 15 | b1-2 | 35 | — | — | 9,400 | 1.61 |
| P-19 | PAG-4 | 15 | a1-2 | 30 | a2-1 | 15 | b1-3 | 40 | — | — | 9,200 | 1.62 |
| P-20 | PAG-6 | 10 | a1-1 | 35 | a2-1 | 15 | b1-1 | 30 | b2-1 | 10 | 9,500 | 1.63 |
| P-21 | PAG-7 | 15 | a1-2 | 35 | a3-1 | 15 | b1-2 | 25 | b2-2 | 10 | 9,100 | 1.63 |
| P-22 | PAG-9 | 15 | a1-1 | 50 | b1-1 | 30 | b2-3 | 5 | — | — | 8,900 | 1.62 |
| P-23 | PAG-1 | 5 | a1-1 | 55 | b1-2 | 40 | — | — | — | — | 9,300 | 1.61 |
| P-24 | PAG-2 | 5 | a1-1 | 30 | a1-3 | 25 | b1-2 | 40 | — | — | 9,100 | 1.62 |
| P-25 | PAG-3 | 5 | a1-2 | 30 | a2-1 | 20 | b1-4 | 35 | b2-1 | 10 | 9,200 | 1.61 |
| P-26 | PAG-7 | 5 | a1-1 | 35 | a3-1 | 15 | b1-1 | 30 | b2-2 | 15 | 9,300 | 1.61 |

TABLE 2

| | | Incorpo- | | Incorpo- | | Incorpo- | | Incorpo- | | Incorpo- | | |
|---|---|---|---|---|---|---|---|---|---|---|---|---|
| | | ration | | ration | | ration | | ration | | ration | | |
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | PAG-A | 15 | a1-1 | 55 | b1-1 | 30 | — | — | — | — | 9,500 | 1.62 |
| CP-2 | PAG-B | 15 | a1-1 | 55 | b1-1 | 30 | — | — | — | — | 9,100 | 1.61 |
| CP-3 | PAG-C | 15 | a1-1 | 55 | b1-1 | 30 | — | — | — | — | 9,300 | 1.63 |
| CP-4 | PAG-D | 15 | a1-1 | 55 | b1-1 | 30 | — | — | — | — | 9,100 | 1.62 |
| CP-5 | PAG-E | 15 | a1-1 | 55 | b1-1 | 30 | — | — | — | — | 9,000 | 1.65 |
| CP-6 | PAG-F | 15 | a1-1 | 55 | b1-1 | 30 | — | — | — | — | 9,500 | 1.62 |
| CP-7 | PAG-B | 15 | a1-2 | 55 | b1-1 | 30 | — | — | — | — | 9,600 | 1.61 |
| CP-8 | PAG-C | 15 | a3-1 | 45 | b1-1 | 40 | — | — | — | — | 9,700 | 1.63 |
| CP-9 | PAG-D | 15 | a1-1 | 55 | b1-3 | 30 | — | — | — | — | 9,500 | 1.63 |
| CP-10 | PAG-E | 15 | a1-1 | 55 | b1-4 | 30 | — | — | — | — | 9,400 | 1.62 |
| CP-11 | PAG-B | 15 | a1-1 | 35 | a3-1 | 15 | b1-2 | 35 | — | — | 9,700 | 1.64 |
| CP-12 | PAG-D | 10 | a1-1 | 35 | a2-1 | 15 | b1-1 | 30 | b2-1 | 10 | 9,500 | 1.62 |
| CP-13 | PAG-C | 15 | a1-2 | 35 | a3-1 | 15 | b1-2 | 25 | b2-2 | 10 | 9,300 | 1.61 |
| CP-14 | PAG-F | 15 | a1-1 | 50 | b1-1 | 30 | b2-3 | 5 | — | — | 9,100 | 1.60 |
| CP-15 | PAG-A | 5 | a1-1 | 55 | b1-2 | 40 | — | — | — | — | 9,300 | 1.62 |
| CP-16 | PAG-D | 5 | a1-2 | 30 | a2-1 | 20 | b1-4 | 35 | b2-1 | 10 | 9,400 | 1.63 |
| CP-17 | a1-1 | 60 | b1-1 | 40 | — | — | — | — | — | — | 5,700 | 1.55 |
| CP-18 | a1-1 | 50 | b1-2 | 30 | b2-1 | 20 | — | — | — | — | 6,100 | 1.54 |

### Preparation of Chemically Amplified Resist Composition

### Examples 3-1 to 3-26 and Comparative Examples 3-1 to 3-18

Chemically amplified resist compositions (R-1 to R-26 and CR-1 to CR-18) were prepared by dissolving a base polymer (P-1 to P-26) containing the sulfonium salt type monomer (PAG-1 to PAG-9) of the present invention, a base polymer (CP-1 to CP-18) containing the comparative sulfonium salt type monomer (PAG-A to PAG-F), a photoacid generator (PAG-X, PAG-Y), and a quencher (Q-1 to Q-4) in a solvent containing 0.01 wt % of a surfactant A (OMNOVA Solutions Inc.) in accordance with the formulation shown in Tables 3 and 4 below to prepare a solution, and filtering the solution through a 0.2 μm Teflon® filter.

TABLE 3

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Example | 3-1 | R-1 | P-1 (80) | Q-1 (8.0) | — | PGMEA (2250) | EL (2800) | DAA (550) |
| | 3-2 | R-2 | P-2 (80) | Q-1 (8.2) | — | PGMEA (2250) | EL (2800) | DAA (550) |
| | 3-3 | R-3 | P-3 (80) | Q-1 (8.0) | — | PGMEA (2250) | EL (2800) | DAA (550) |
| | 3-4 | R-4 | P-4 (80) | Q-1 (7.8) | — | PGMEA (2250) | EL (2800) | DAA (550) |
| | 3-5 | R-5 | P-5 (80) | Q-1 (8.0) | — | PGMEA (2250) | EL (2800) | DAA (550) |
| | 3-6 | R-6 | P-6 (80) | Q-1 (8.0) | — | PGMEA (2250) | EL (2800) | DAA (550) |
| | 3-7 | R-7 | P-7 (80) | Q-1 (8.2) | — | PGMEA (2250) | EL (2800) | DAA (550) |
| | 3-8 | R-8 | P-8 (80) | Q-1 (8.0) | — | PGMEA (2250) | EL (2800) | DAA (550) |
| | 3-9 | R-9 | P-9 (80) | Q-1 (7.6) | — | PGMEA (2250) | EL (2800) | DAA (550) |
| | 3-10 | R-10 | P-10 (80) | Q-2 (8.2) | — | PGMEA (2250) | EL (2800) | DAA (550) |
| | 3-11 | R-11 | P-11 (80) | Q-3 (8.0) | — | PGMEA (2250) | EL (2800) | DAA (550) |
| | 3-12 | R-12 | P-12 (80) | Q-1 (8.0) | — | PGMEA (2250) | EL (2800) | DAA (550) |
| | 3-13 | R-13 | P-13 (80) | Q-1 (8.2) | — | PGMEA (2250) | EL (2800) | DAA (550) |
| | 3-14 | R-14 | P-14 (80) | Q-1 (8.0) | — | PGMEA (2250) | EL (2800) | DAA (550) |
| | 3-15 | R-15 | P-15 (80) | Q-3 (7.8) | — | PGMEA (2250) | EL (2800) | DAA (550) |
| | 3-16 | R-16 | P-16 (80) | Q-2 (8.0) | — | PGMEA (2250) | EL (2800) | DAA (550) |
| | 3-17 | R-17 | P-17 (80) | Q-1 (8.0) | — | PGMEA (2250) | EL (2800) | DAA (550) |
| | 3-18 | R-18 | P-18 (80) | Q-1 (7.8) | — | PGMEA (2250) | EL (2800) | DAA (550) |
| | 3-19 | R-19 | P-19 (80) | Q-2 (8.0) | — | PGMEA (2250) | EL (2800) | DAA (550) |
| | 3-20 | R-20 | P-20 (80) | Q-3 (7.8) | PAG-Y (15) | PGMEA (2250) | EL (2800) | DAA (550) |
| | 3-21 | R-21 | P-21 (80) | Q-1 (4.0) | — | PGMEA (2250) | EL (2800) | DAA (550) |
| | | | | Q-4 (3.8) | | | | |
| | 3-22 | R-22 | P-22 (80) | Q-1 (8.2) | — | PGMEA (2250) | EL (2800) | DAA (550) |
| | 3-23 | R-23 | P-23 (80) | Q-3 (7.6) | — | PGMEA (2250) | EL (2800) | DAA (550) |
| | 3-24 | R-24 | P-24 (80) | Q-1 (8.0) | PAG-X (10) | PGMEA (2250) | EL (2800) | DAA (550) |
| | 3-25 | R-25 | P-25 (80) | Q-2 (8.2) | PAG-X (10) | PGMEA (2250) | EL (2800) | DAA (550) |
| | 3-26 | R-26 | P-26 (80) | Q-3 (8.0) | PAG-Y (15) | PGMEA (2250) | EL (2800) | DAA (550) |

TABLE 4

| | | | | | Photoacid | | | |
|---|---|---|---|---|---|---|---|---|
| | | Resist | Base polymer | Quencher | generator | Solvent 1 | Solvent 2 | Solvent 3 |
| | | composition | (pbw) | (pbw) | (pbw) | (pbw) | (pbw) | (pbw) |
| Comparative | 3-1 | CR-1 | CP-1 (80) | Q-1 (8.0) | — | PGMEA (2250) | EL (2800) | DAA (550) |
| Example | 3-2 | CR-2 | CP-2 (80) | Q-1 (8.2) | — | PGMEA (2250) | EL (2800) | DAA (550) |
| | 3-3 | CR-3 | CP-3 (80) | Q-1 (8.0) | — | PGMEA (2250) | EL (2800) | DAA (550) |
| | 3-4 | CR-4 | CP-4 (80) | Q-1 (7.8) | — | PGMEA (2250) | EL (2800) | DAA (550) |
| | 3-5 | CR-5 | CP-5 (80) | Q-1 (8.0) | — | PGMEA (2250) | EL (2800) | DAA (550) |
| | 3-6 | CR-6 | CP-6 (80) | Q-1 (8.2) | — | PGMEA (2250) | EL (2800) | DAA (550) |
| | 3-7 | CR-7 | CP-7 (80) | Q-2 (8.2) | — | PGMEA (2250) | EL (2800) | DAA (550) |
| | 3-8 | CR-8 | CP-8 (80) | Q-1 (8.2) | — | PGMEA (2250) | EL (2800) | DAA (550) |
| | 3-9 | CR-9 | CP-9 (80) | Q-3 (7.8) | — | PGMEA (2250) | EL (2800) | DAA (550) |
| | 3-10 | CR-10 | CP-10 (80) | Q-2 (8.0) | — | PGMEA (2250) | EL (2800) | DAA (550) |
| | 3-11 | CR-11 | CP-11 (80) | Q-1 (8.0) | — | PGMEA (2250) | EL (2800) | DAA (550) |
| | 3-12 | CR-12 | CP-12 (80) | Q-3 (7.8) | PAG-Y (15) | PGMEA (2250) | EL (2800) | DAA (550) |
| | 3-13 | CR-13 | CP-13 (80) | Q-1 (4.0) | — | PGMEA (2250) | EL (2800) | DAA (550) |
| | | | | Q-4 (3.8) | | | | |
| | 3-14 | CR-14 | CP-14 (80) | Q-1 (8.2) | — | PGMEA (2250) | EL (2800) | DAA (550) |
| | 3-15 | CR-15 | CP-15 (80) | Q-3 (7.6) | — | PGMEA (2250) | EL (2800) | DAA (550) |
| | 3-16 | CR-16 | CP-16 (80) | Q-2 (8.2) | PAG-X (10) | PGMEA (2250) | EL (2800) | DAA (550) |
| | 3-17 | CR-17 | CP-17 (80) | Q-1 (8.0) | PAG-X (24) | PGMEA (2250) | EL (2800) | DAA (550) |
| | 3-18 | CR-18 | CP-18 (80) | Q-1 (8.0) | PAG-Y (24) | PGMEA (2250) | EL (2800) | DAA (550) |

In Tables 3 and 4, the solvents, the photoacid generators PAG-X and PAG-Y, the quenchers Q-1 to Q-4, and the surfactant A are as follows.

### Solvents:

PGMEA (propylene glycol monomethyl ether acetate)
EL (ethyl lactate)
DAA (diacetone alcohol)
   Photoacid generators: PAG-X and PAG-Y

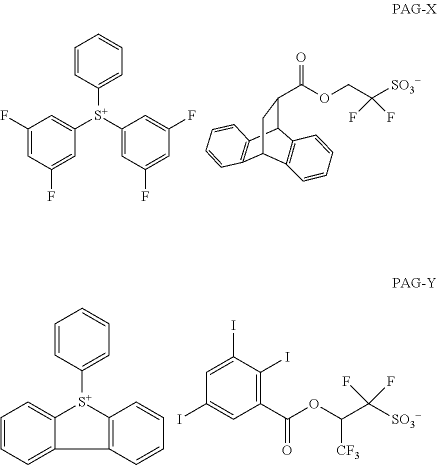

### Quenchers: Q-1 to Q-4

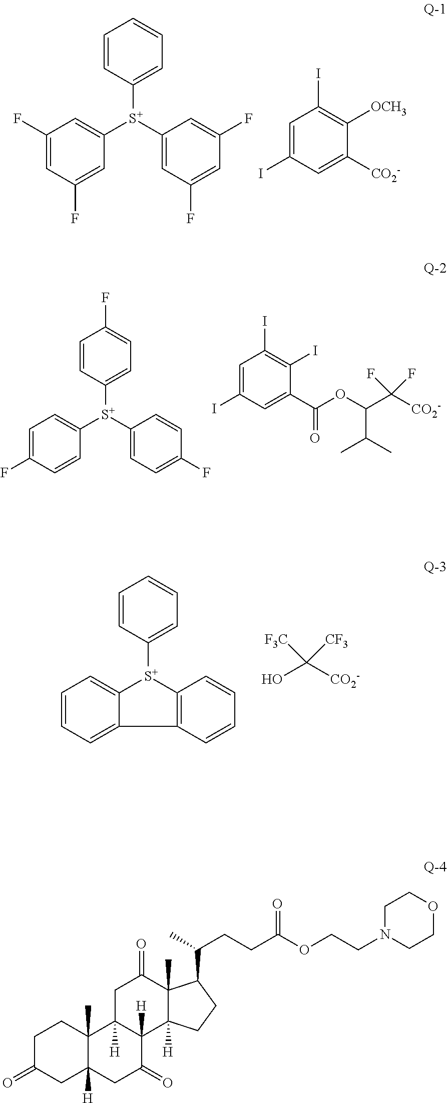

### Surfactant A:

3-methyl-3-(2,2,2-trifluoroethoxymethyl) oxetane/tetrahydrofuran/2,2-dimethyl-1,3-propanediol copolymer (manufactured by OMNOVA Solutions Inc.)

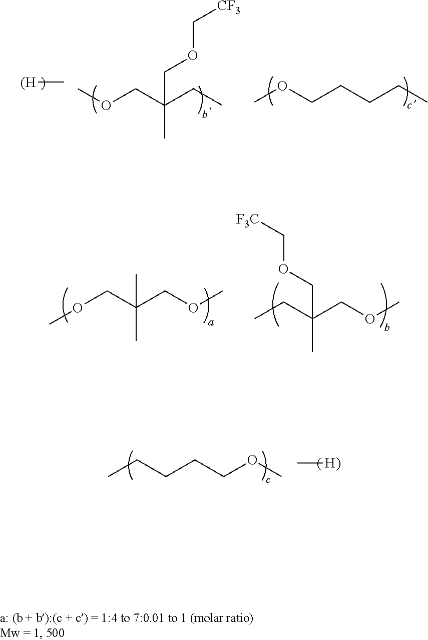

EUV lithography test (1)

### Examples 4-1 to 4-26 and Comparative Examples 4-1 to 4-18

Each chemically amplified resist composition (R-1 to R-26 and CR-1 to CR-18) shown in Tables 3 and 4 was spin coated on a Si substrate having a 20-nm coating of silicon-containing spin-on hard mask SHB-A940 manufactured by Shin-Etsu Chemical Co., Ltd. (content of silicon: 43 wt %) and prebaked on a hotplate at 100° C. for 60 seconds to form a resist film of 50 nm thick. Using an EUV scanner NXE3400 (NA 0.33, o 0.9/0.6, dipole illumination) manufactured by ASML, the resist film was exposed to EUV through a LS pattern having a size of 18 nm and a pitch of 36 nm (on-wafer size) while varying the exposure dose and focus (exposure dose pitch: 1 mJ/cm², focus pitch: 0.020 μm), and after the exposure, the resist film was subjected to PEB at the temperature shown in Tables 5 and 6 for 60 seconds. Thereafter, the resist film was puddle developed in a 2.38 wt % TMAH aqueous solution for 30 seconds, rinsed with a surfactant-containing rinse material, and spin-dried to obtain a positive pattern.

The obtained LS pattern was observed with a critical dimension SEM (CG6300) manufactured by Hitachi High-Tech Corporation and evaluated for sensitivity, EL, LWR, depth of focus (DOF), and collapse limit by the following methods. The development defects of the obtained LS pattern were evaluated. The results are shown in Tables 5 and 6.

### [Sensitivity Evaluation]

An optimum exposure dose Eop (mJ/cm²) which provided a LS pattern with a line width of 18 nm and a pitch of 36 nm was determined and taken as sensitivity. A smaller value indicates higher sensitivity.

### [EL Evaluation]

EL (unit: %) was determined from the exposure dose which provided a LS pattern with a space width of 18 nm±10% (16.2 to 19.8 nm) according to the following equation. A greater value indicates better performance.

$$EL ( \% ) = ( \vert {E}_{1} - {E}_{2} \vert / Eop ) \times 100$$

E₁: optimum exposure dose which provides a LS pattern with a line width of 16.2 nm and a pitch of 36 nm

E₂: optimum exposure dose which provides a LS pattern with a line width of 19.8 nm and a pitch of 36 nm

Eop: optimum exposure dose which provides a LS pattern with a line width of 18 nm and a pitch of 36 nm

### [LWR Evaluation]

For the LS pattern formed by exposure at the optimum exposure dose Eop, the line width was measured at 10 longitudinally spaced apart points, from which a 3-fold value (36) of the standard deviation (6) was determined as LWR. As this value is smaller, a pattern having small roughness and uniform line width can be obtained.

### [DOF Evaluation]

As evaluation of the depth of focus, a range of focus which provided a LS pattern with a size of 18 nm±10% (16.2 to 19.8 nm) was determined. A greater value indicates a wider depth of focus.

### [Evaluation of Collapse Limit of Line Pattern]

For the LS pattern formed by exposure at the exposure dose corresponding to the optimum focus, the line width was measured at 10 longitudinally spaced apart points. The minimum line size above which lines could be resolved without collapse was determined and reported as collapse limit. A smaller value indicates better collapse limit.

### [Development Defect Evaluation]

Using a defect inspection apparatus KLA2360 (trade name) manufactured by KLA-Tencor Corporation, the LS pattern with a line width of 18 nm and a pitch of 36 nm formed with the optimum exposure dose was set to a pixel size of 0.16 μm and a threshold value of 20 in the defect inspection apparatus, and the number of defects (number/cm²) extracted from the difference caused by the superimposition on the comparative image in units of pixels was detected to calculate the number of defects (number/cm²) per unit area. Thereafter, development defects were classified and extracted from all defects by defect review, and the number of development defects (number/cm²) per unit area was calculated. A case where the value was less than 0.5 was rated as A, a case where the value was 0.5 or more and less than 1.0 was rated as B, a case where the value was 1.0 or more and less than 5.0 was rated as C, and a case where the value was 5.0 or more was rated as D. A smaller value indicates better performance.

TABLE 5

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Example | 4-1 | R-1 | 95 | 32 | 18 | 2.2 | 120 | 10.9 | A |
| | 4-2 | R-2 | 100 | 33 | 19 | 2.3 | 110 | 10.8 | A |
| | 4-3 | R-3 | 100 | 32 | 17 | 2.3 | 120 | 10.8 | A |
| | 4-4 | R-4 | 95 | 32 | 18 | 2.4 | 110 | 11.3 | A |
| | 4-5 | R-5 | 105 | 33 | 17 | 2.2 | 100 | 11.1 | A |
| | 4-6 | R-6 | 100 | 35 | 17 | 2.3 | 120 | 11.2 | A |
| | 4-7 | R-7 | 95 | 34 | 18 | 2.3 | 110 | 11.2 | A |
| | 4-8 | R-8 | 95 | 32 | 19 | 2.3 | 100 | 11.3 | A |
| | 4-9 | R-9 | 100 | 33 | 17 | 2.4 | 110 | 11.4 | A |
| | 4-10 | R-10 | 100 | 34 | 18 | 2.3 | 120 | 11.1 | A |
| | 4-11 | R-11 | 100 | 32 | 19 | 2.4 | 120 | 11.0 | A |
| | 4-12 | R-12 | 95 | 34 | 17 | 2.4 | 110 | 10.7 | A |
| | 4-13 | R-13 | 105 | 33 | 18 | 2.2 | 120 | 10.8 | A |
| | 4-14 | R-14 | 100 | 32 | 17 | 2.3 | 100 | 10.7 | A |
| | 4-15 | R-15 | 95 | 33 | 17 | 2.4 | 110 | 10.9 | A |
| | 4-16 | R-16 | 95 | 33 | 18 | 2.3 | 120 | 11.3 | A |
| | 4-17 | R-17 | 100 | 32 | 17 | 2.5 | 110 | 11.1 | A |
| | 4-18 | R-18 | 95 | 34 | 18 | 2.3 | 110 | 11.0 | A |
| | 4-19 | R-19 | 95 | 33 | 18 | 2.4 | 120 | 11.2 | A |
| | 4-20 | R-20 | 100 | 34 | 17 | 2.4 | 110 | 10.9 | A |
| | 4-21 | R-21 | 100 | 35 | 19 | 2.3 | 110 | 10.8 | A |
| | 4-22 | R-22 | 100 | 32 | 17 | 2.2 | 110 | 11.1 | A |
| | 4-23 | R-23 | 95 | 33 | 18 | 2.4 | 110 | 11.2 | A |
| | 4-24 | R-24 | 95 | 32 | 18 | 2.4 | 120 | 11.1 | A |
| | 4-25 | R-25 | 100 | 34 | 17 | 2.3 | 110 | 11.0 | A |
| | 4-26 | R-26 | 100 | 32 | 19 | 2.2 | 110 | 10.9 | A |

TABLE 6

| | | | | Optimum | | | | | |
|---|---|---|---|---|---|---|---|---|---|
| | | | PEB | exposure | | | | Collapse | |
| | | Resist | temp. | dose | EL | LWR | DOF | limit | Development |
| | | composition | (° C.) | (mJ/cm²) | (%) | (nm) | (nm) | (nm) | defects |
| Comparative | 4-1 | CR-1 | 95 | 40 | 13 | 3.1 | 90 | 12.9 | B |
| Example | 4-2 | CR-2 | 100 | 38 | 14 | 2.7 | 80 | 12.5 | C |
| | 4-3 | CR-3 | 100 | 38 | 14 | 2.8 | 80 | 13.2 | C |
| | 4-4 | CR-4 | 100 | 37 | 15 | 2.9 | 90 | 13.6 | B |
| | 4-5 | CR-5 | 95 | 39 | 13 | 2.8 | 80 | 12.3 | C |
| | 4-6 | CR-6 | 100 | 36 | 11 | 3.4 | 60 | 12.5 | B |
| | 4-7 | CR-7 | 100 | 38 | 14 | 2.7 | 90 | 12.6 | C |
| | 4-8 | CR-8 | 100 | 39 | 13 | 2.9 | 80 | 12.4 | C |
| | 4-9 | CR-9 | 95 | 38 | 15 | 3.0 | 90 | 13.3 | B |
| | 4-10 | CR-10 | 100 | 38 | 14 | 2.9 | 80 | 12.4 | B |
| | 4-11 | CR-11 | 100 | 37 | 14 | 2.9 | 80 | 12.2 | B |
| | 4-12 | CR-12 | 100 | 39 | 14 | 2.9 | 80 | 13.4 | B |
| | 4-13 | CR-13 | 95 | 37 | 13 | 2.7 | 90 | 12.2 | B |
| | 4-14 | CR-14 | 105 | 35 | 11 | 3.3 | 50 | 12.7 | C |
| | 4-15 | CR-15 | 100 | 40 | 13 | 3.5 | 70 | 12.7 | C |
| | 4-16 | CR-16 | 95 | 38 | 15 | 2.8 | 80 | 13.2 | B |
| | 4-17 | CR-17 | 95 | 41 | 10 | 3.3 | 60 | 12.4 | B |
| | 4-18 | CR-18 | 100 | 40 | 10 | 3.2 | 60 | 12.6 | B |

From the results shown in Tables 5 and 6, it was confirmed that the chemically amplified resist composition comprising a polymer composed of the sulfonium salt type monomer of the present invention has favorable sensitivity and is excellent in EL, LWR, and DOF. It was confirmed that the value of the collapse limit was small and the pattern was resistant to collapse even in fine pattern formation. It was confirmed that development defects were also suppressed. Therefore, it was shown that the chemically amplified resist composition of the present invention is suitable as a material for EUV lithography.

### EUV Lithography Test (2)

### Examples 5-1 to 5-26 and Comparative Examples 5-1 to 5-18

Each chemically amplified resist composition (R-1 to R-26 and CR-1 to CR-18) shown in Tables 3 and 4 was spin coated on a Si substrate having a 20-nm coating of silicon-containing spin-on hard mask SHB-A940 manufactured by Shin-Etsu Chemical Co., Ltd. (content of silicon: 43 wt %) and prebaked on a hotplate at 105° C. for 60 seconds to form a resist film of 50 nm thick. Using an EUV scanner NXE3400 (NA 0.33, @ 0.9/0.6, quadrupole illumination, mask bearing a hole pattern at a pitch 46 nm (on-wafer size) and +20% bias) manufactured by ASML, the resist film was exposed to EUV, the resist film was baked (PEB) on a hotplate at the temperature shown in Tables 7 and 8 for 60 seconds and developed in a 2.38 wt % TMAH aqueous solution for 30 seconds to form a hole pattern having a size of 23 nm.

The resist pattern was observed under CD-SEM (CG6300, Hitachi High-Technologies Corp.). The exposure dose that provides a hole pattern having a size of 23 nm was reported as sensitivity. The size of 50 holes printed at that dose was measured, from which a 3-fold value (36) of the standard deviation (6) was computed and reported as CDU. The results are shown in Tables 7 and 8.

TABLE 7

| | | | PEB | Optimum | |
|---|---|---|---|---|---|
| | | Resist | temp. | exposure dose | CDU |
| | | composition | (° C.) | (mJ/cm²) | (nm) |
| Example | 5-1 | R-1 | 95 | 22 | 2.2 |
| | 5-2 | R-2 | 95 | 23 | 2.4 |
| | 5-3 | R-3 | 90 | 24 | 2.3 |
| | 5-4 | R-4 | 90 | 23 | 2.2 |
| | 5-5 | R-5 | 90 | 22 | 2.4 |
| | 5-6 | R-6 | 95 | 23 | 2.4 |
| | 5-7 | R-7 | 95 | 23 | 2.5 |
| | 5-8 | R-8 | 90 | 24 | 2.3 |
| | 5-9 | R-9 | 95 | 24 | 2.2 |
| | 5-10 | R-10 | 95 | 23 | 2.3 |
| | 5-11 | R-11 | 95 | 23 | 2.3 |
| | 5-12 | R-12 | 90 | 24 | 2.4 |
| | 5-13 | R-13 | 90 | 24 | 2.2 |
| | 5-14 | R-14 | 90 | 24 | 2.4 |
| | 5-15 | R-15 | 90 | 22 | 2.3 |
| | 5-16 | R-16 | 85 | 24 | 2.3 |
| | 5-17 | R-17 | 95 | 23 | 2.2 |
| | 5-18 | R-18 | 95 | 24 | 2.3 |
| | 5-19 | R-19 | 90 | 25 | 2.4 |
| | 5-20 | R-20 | 95 | 23 | 2.3 |
| | 5-21 | R-21 | 95 | 22 | 2.3 |
| | 5-22 | R-22 | 95 | 23 | 2.4 |
| | 5-23 | R-23 | 95 | 24 | 2.2 |
| | 5-24 | R-24 | 90 | 23 | 2.4 |
| | 5-25 | R-25 | 95 | 22 | 2.3 |
| | 5-26 | R-26 | 95 | 23 | 2.2 |

TABLE 8

| | | | PEB | Optimum | |
|---|---|---|---|---|---|
| | | Resist | temp. | exposure dose | CDU |
| | | composition | (° C.) | (mJ/cm²) | (nm) |
| Comparative | 5-1 | CR-1 | 95 | 31 | 3.0 |
| Example | 5-2 | CR-2 | 95 | 28 | 2.9 |
| | 5-3 | CR-3 | 95 | 27 | 2.7 |
| | 5-4 | CR-4 | 90 | 27 | 2.8 |
| | 5-5 | CR-5 | 90 | 28 | 2.7 |
| | 5-6 | CR-6 | 95 | 25 | 3.5 |
| | 5-7 | CR-7 | 90 | 27 | 2.7 |
| | 5-8 | CR-8 | 90 | 27 | 2.7 |
| | 5-9 | CR-9 | 90 | 26 | 2.7 |
| | 5-10 | CR-10 | 95 | 29 | 2.8 |
| | 5-11 | CR-11 | 95 | 29 | 2.7 |
| | 5-12 | CR-12 | 95 | 29 | 2.9 |
| | 5-13 | CR-13 | 85 | 27 | 2.8 |
| | 5-14 | CR-14 | 95 | 25 | 3.3 |
| | 5-15 | CR-15 | 95 | 31 | 3.2 |
| | 5-16 | CR-16 | 90 | 29 | 2.8 |
| | 5-17 | CR-17 | 95 | 32 | 3.1 |
| | 5-18 | CR-18 | 95 | 32 | 3.2 |

From the results shown in Tables 7 and 8. it was confirmed that the chemically amplified resist composition comprising a polymer composed of the sulfonium salt type monomer of the present invention has favorable sensitivity and is excellent in CDU.

### Dry Etching Resistance Evaluation

### Examples 6-1 to 6-26 and Comparative Examples 6-1 to 6-18

2 g of each of the polymers shown in Tables 1 and 2 (Polymers P-1 to P-26 and Comparative Polymers CP-1 to CP-18) was dissolved in 10 g of cyclohexanone, the polymer solution was filtered through a filter with a pore size of 0.2 μm, and spin-coated on a Si substrate to form a 300 nm-thick film thereon, and the film was evaluated under the following conditions.

Etching Test with CHF₃/CF₄-Based Gas:

A polymer film thickness difference before and after etching was determined using a dry etching apparatus TE-8500P manufactured by Tokyo Electron Limited.

The etching conditions are shown below.

| | | |
|---|---|---|
| Chamber pressure | 40 | Pa |
| RF power | 1000 | W |
| Gap | 9 | mm |
| CHF₃ gas flow rate | 30 | mL/min |
| CF₄ gas flow rate | 30 | mL/min |
| Ar gas flow rate | 100 | mL/min |
| Time | 60 | sec |

In this evaluation, a smaller film thickness difference, that is, a smaller amount of decrease, indicates higher etching resistance.

The results of dry etching resistance are shown in Tables 9 and 10.

TABLE 9

| | | | CHF₃/CF₄-based gas |
|---|---|---|---|
| | | Polymer | etching rate (nm/min) |
| Example | 6-1 | P-1 | 96 |
| | 6-2 | P-2 | 97 |
| | 6-3 | P-3 | 96 |
| | 6-4 | P-4 | 96 |
| | 6-5 | P-5 | 95 |
| | 6-6 | P-6 | 96 |
| | 6-7 | P-7 | 95 |
| | 6-8 | P-8 | 95 |
| | 6-9 | P-9 | 97 |
| | 6-10 | P-10 | 95 |
| | 6-11 | P-11 | 96 |
| | 6-12 | P-12 | 96 |
| | 6-13 | P-13 | 96 |
| | 6-14 | P-14 | 95 |
| | 6-15 | P-15 | 96 |
| | 6-16 | P-16 | 95 |
| | 6-17 | P-17 | 96 |
| | 6-18 | P-18 | 95 |
| | 6-19 | P-19 | 96 |
| | 6-20 | P-20 | 95 |
| | 6-21 | P-21 | 96 |
| | 6-22 | P-22 | 96 |
| | 6-23 | P-23 | 95 |
| | 6-24 | P-24 | 97 |
| | 6-25 | P-25 | 95 |
| | 6-26 | P-26 | 96 |

TABLE 10

| | | | CHF₃/CF₄-based gas |
|---|---|---|---|
| | | Polymer | etching rate (nm/min) |
| Comparative | 6-1 | CP-1 | 114 |
| Example | 6-2 | CP-2 | 98 |
| | 6-3 | CP-3 | 97 |
| | 6-4 | CP-4 | 98 |
| | 6-5 | CP-5 | 109 |
| | 6-6 | CP-6 | 111 |
| | 6-7 | CP-7 | 99 |
| | 6-8 | CP-8 | 97 |
| | 6-9 | CP-9 | 97 |
| | 6-10 | CP-10 | 105 |
| | 6-11 | CP-11 | 98 |
| | 6-12 | CP-12 | 97 |
| | 6-13 | CP-13 | 97 |
| | 6-14 | CP-14 | 112 |
| | 6-15 | CP-15 | 109 |
| | 6-16 | CP-16 | 99 |
| | 6-17 | CP-17 | 104 |
| | 6-18 | CP-18 | 101 |

From the results shown in Tables 9 and 10, it was confirmed that the polymer of the present invention has excellent dry etching resistance to a CHF₃/CF₄-based gas.

Japanese Patent Application No. 2024-131989 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A sulfonium salt type monomer having the following formula (A): 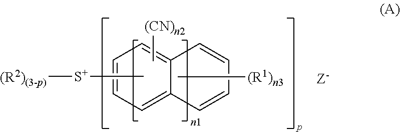
wherein p is 1, 2, or 3, n1 is 0 or 1, n2 is 1 or 2, n3 is 0, 1, 2, or 3, provided that, when n1 is 0, 1≤n2+n3≤5, and when n1 is 1, 1≤n2+n3≤7,
R¹ is a halogen atom, a nitro group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, when n3 is 2 or 3, respective R¹ may be the same as or different from each other, and two R′'s may bond together to form a ring with the carbon atom to which they are attached,
R² is a halogen atom, or a C₁-C₃₀ hydrocarbyl group which may contain a heteroatom, when p is 1, two R²'s may be the same as or different from each other, two of three substituents bonded to S⁺ may bond together to form a ring with the sulfur atom to which they are bonded, and
Z⁻ is an aromatic sulfonic acid anion having an aromatic vinyl structure.

2. The sulfonium salt type monomer according to claim 1, which has the following formula (A1): 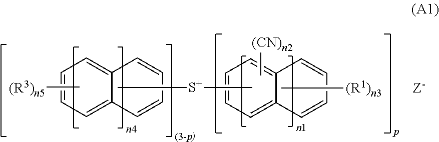
wherein p, n1 to n3, R¹, and Z⁻ are as defined above,
n4 or 1, n5 is 0, 1, 2, 3, 4, or 5,
R³ is a halogen atom, a nitro group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, and when n5 is 2, 3, 4, or 5, respective R³ may be the same as or different from each other, and two R³'s may bond together to form a ring with the carbon atom to which they are attached.

3. The sulfonium salt type monomer according to claim 1, wherein Z⁻ is an anion having the following formula (Z): 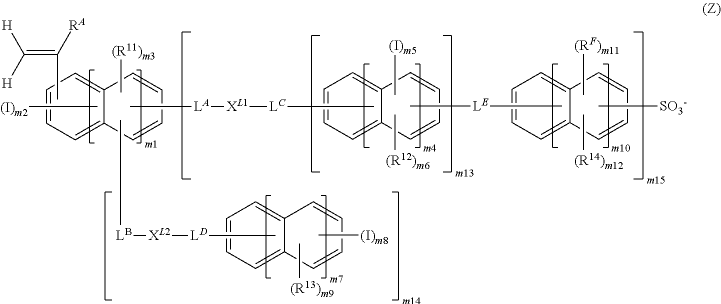
wherein m1 is 0 or 1, m2 is 0, 1, 2, 3, or 4, m3 is 0, 1, 2, or 3, m4 is 0 or 1, m5 is 0, 1, 2, 3, or 4, m6 is 0, 1, 2, or 3, m7 is 0 or 1, m8 is 1, 2, 3, or 4, m9 is 0, 1, 2, or 3, m10 is 0 or 1, m11 is 0, 1, 2, 3, or 4, m12 is 0, 1, 2, or 3, m13 is 0 or 1, m14 is 0 or 1, m15 is 0 or 1, provided that, when m1 is 0, 0≤m2+m3+m14≤4, and when m1 is 1, 0≤m2+m3+m14≤6, when m4 is 0, 0≤m5+m6≤4, and when m4 is 1, 0≤m5+m6≤6, when m7 is 0, 0≤m8+m9≤5, and when m7 is 1, 0≤m8+m9≤7, when m10 is 0, 0≤m11+m12≤ 4, and when m10 is 1, 0≤m11+m12≤6, and 1≤m2+m5+m8≤4,
R^{A} is a hydrogen atom, a fluorine atom, a methyl group, or a trifluoromethyl group,
R¹¹, R¹², and R₁₃ are each independently a halogen atom other than an iodine atom, a nitro group, a cyano group, a hydroxy group, a carboxy group, a C₁-C₂₀ hydrocarbyl group which may contain a heteroatom, a C₁-C₂₀ hydrocarbyloxy group which may contain a heteroatom, a C₁-C₂₀ hydrocarbylthio group which may contain a heteroatom, or a C₂-C₂₀ hydrocarbyloxycarbonyl group which may contain a heteroatom, when m3 is 2 or 3, respective R¹¹ may be the same as or different from each other, and two R¹¹'s may bond together to form a ring with the carbon atom to which they are attached, when m6 is 2 or 3, respective R¹² may be the same as or different from each other, and two R¹²'s may bond together to form a ring with the carbon atom to which they are attached, when m9 is 2 or 3, respective R¹³ may be the same as or different from each other, and two R¹³'s may bond together to form a ring with the carbon atom to which they are attached,
R¹⁴ is a halogen atom other than a fluorine atom and an iodine atom, a nitro group, a hydroxy group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m12 is 2 or 3, respective R¹⁴ may be the same as or different from each other, and two R¹⁴'s may bond together to form a ring with the carbon atom to which they are attached,
R^{F} is a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, when m11 is 2, 3, or 4, respective R^{F} may be the same as or different from each other,
L^{A}, L^{B}, L^{C}, L^{D}, and L^{E} are each independently a single bond, an ether bond, an ester bond, a sulfonic acid ester bond, an amide bond, a sulfonic acid amide bond, a carbonate bond, or a carbamate bond, and
X^{L1} and X^{L2} are each independently a single bond or a C₁-C₄₀ hydrocarbylene group which may contain a heteroatom,
provided that m13 and m14 are not 0 at the same time, and L^{A}, L^{B}, L^{C}, L^{D}, X^{L1}, and X^{L2} are not a single bond at the same time.

4. The sulfonium salt type monomer according to claim 3, wherein m15 is 1.

5. A monomeric photoacid generator comprising the sulfonium salt type monomer according to claim 1.

6. A polymer comprising repeat units derived from the monomeric photoacid generator according to claim 5.

7. The polymer according to claim 6, further comprising at least one selected from repeat units having the following formula (a1), repeat units having the following formula (a2), and repeat units having the following formula (a3): 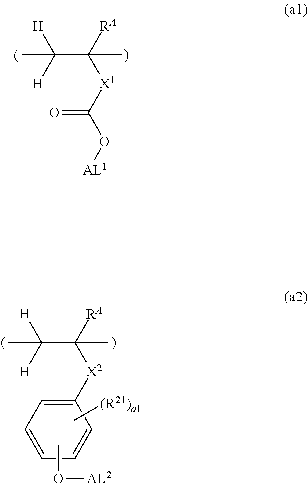
wherein R^{A} is each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted with a hydroxy group, a nitro group, a cyano group, a C₁-C₁₀ saturated hydrocarbyl group which may contain a fluorine atom, a C₁-C₁₀ saturated hydrocarbyloxy group which may contain a fluorine atom, or a halogen atom, X¹¹ is a C₁-C₁₀ saturated hydrocarbylene group, a phenylene group, or a naphthylene group, the saturated hydrocarbylene group may contain a hydroxy group, an ether bond, an ester bond, or a lactone ring,
X² is a single bond, *—C(═O)—O—, or *—C(═O)—NH—,
* designates a point of attachment to the carbon atom in the backbone,
R²¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when a1 is 2, 3, or 4, respective R²¹ may be the same as or different from each other,
AL¹ and AL² are each independently an acid labile group, and
a1 is 0, 1, 2, 3, or 4, and 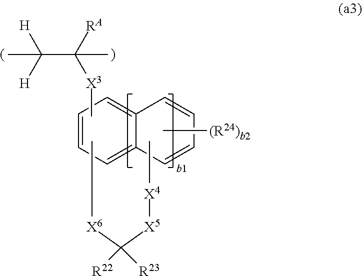
wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or groups obtained by combining these,
X⁵ and X⁶ are each independently an oxygen atom or a sulfur atom, provided that X⁴ and X⁶ bond to adjacent carbon atoms of an aromatic ring,
R²² and R²³ are each independently a hydrogen atom, or a C₁-C₂₀ hydrocarbyl group which may contain a heteroatom, R²² and R²³ may bond together to form a ring with the carbon atom to which they are attached,
R²⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{24A})(R^{24B}), R^{24A} and R^{24B} are each independently a hydrogen atom or a C₁-C₆ hydrocarbyl group, and when b2 is 2 or more, respective R²⁴ may be the same as or different from each other, and a plurality of R²⁴'s may bond together to form a ring with the carbon atom of an aromatic ring to which they are attached.

8. The polymer according to claim 6, further comprising at least one selected from repeat units having the following formula (b1) and repeat units having the following formula (b2): 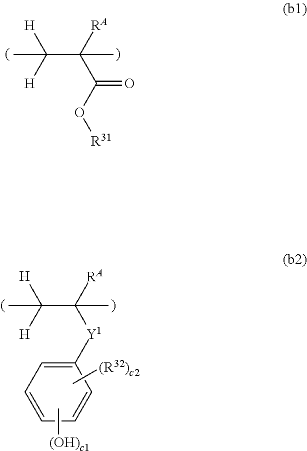
wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R³¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—),
R³² is a halogen atom, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, when c2 is 2, 3, or 4, respective R³² may be the same as or different from each other,
c1 is 1, 2, 3, or 4, and c2 is 0, 1, 2, 3, or 4, provided that 1≤c1+c2≤5.

9. A chemically amplified resist composition comprising (A) a base polymer containing the polymer according to claim 6.

10. The chemically amplified resist composition according to claim 9, further comprising (B) an organic solvent.

11. The chemically amplified resist composition according to claim 9, further comprising (C) a quencher.

12. The chemically amplified resist composition according to claim 9, further comprising (D) a photoacid generator.

13. The chemically amplified resist composition according to claim 9, further comprising (E) a surfactant.

14. A pattern forming process comprising the steps of: applying the chemically amplified resist composition according to claim 9 onto a substrate to form a resist film thereon;
exposing the resist film to high-energy radiation; and developing the exposed resist film in a developer.

15. The pattern forming process according to claim 14, wherein the high-energy radiation is KrF excimer laser, ArF excimer laser, electron beam, or extreme ultraviolet having a wavelength of 3 to 15 nm.
